(19) United States
(12) Patent Application Publication
(10) Pub. No.: US 2022/0313698 A1
(43) Pub. Date: Oct. 6, 2022

(54) MODULATORS OF CYSTIC FIBROSIS TRANSMEMBRANE CONDUCTANCE REGULATOR

(71) Applicant: Vertex Pharmaceuticals Incorporated, Boston, MA (US)

(72) Inventors: Alexander Russell Abela, Escondido, CA (US); Corey Don Anderson, Brighton, MA (US); Brett C. Bookser, San Diego, CA (US); Brett B. Busch, San Diego, CA (US); Jeremy J. Clemens, San Diego, CA (US); Thomas Cleveland, San Marcos, CA (US); Timothy Richard Coon, Carlsbad, CA (US); Bryan Frieman, La Jolla, CA (US); Senait G. Ghirmai, San Diego, CA (US); Peter Grootenhuis, Del Mar, CA (US); Anton V. Gulevich, San Diego, CA (US); Sara Sabina Hadida Ruah, La Jolla, CA (US); Yoshihiro Ishihara, San Diego, CA (US); Haripada Khatuya, San Diego, CA (US); Jason McCartney, Cardiff by the Sea, CA (US); Mark Thomas Miller, Rancho Santa Fe, CA (US); Prasuna Paraselli, San Diego, CA (US); Fabrice Pierre, La Jolla, CA (US); Andreas Termin, Encinitas, CA (US); Sara E. Swift, San Diego, CA (US); Johnny Uy, San Diego, CA (US); Carl V. Vogel, Carlsbad, CA (US); Jinglan Zhou, San Diego, CA (US)

(73) Assignee: Vertex Pharmaceuticals Incorporated

(21) Appl. No.: 16/992,675

(22) Filed: Aug. 13, 2020

Related U.S. Application Data
(60) Provisional application No. 62/886,611, filed on Aug. 14, 2019.
(60) Provisional application No. 62/886,739, filed on Aug. 14, 2019.

(51) Int. Cl. A61K 31/529; A61K 31/404; A61K 31/47; A61K 31/4418; A61K 31/439; C07D 513/22

(52) U.S. Cl. CPC A61K 31/529; A61K 31/404; C07D 513/22; A61K 31/4418; A61K 31/439; A61K 31/47

(57) ABSTRACT

This disclosure provides modulators of Cystic Fibrosis Transmembrane Conductance Regulator (CFTR), pharmaceutical compositions containing at least one such modulator, methods of treatment of cystic fibrosis using such modulators and pharmaceutical compositions, and processes for making such modulators.

This application claims the benefit of U.S. Provisional Application No. 62/886,611, filed on Aug. 14, 2019, and U.S. Provisional Application No. 62/886,739, filed on Aug. 14, 2019, the disclosures of which are incorporated by reference in its entirety.

The invention relates to modulators of Cystic Fibrosis Transmembrane Conductance Regulator (CFTR), pharmaceutical compositions containing the modulators, methods of treatment of cystic fibrosis using such modulators and pharmaceutical compositions, and processes for making such modulators.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 432 of these identified mutations, with sufficient evidence to define 352 mutations as disease causing. The most prevalent disease-causing mutation is a deletion of phenylalanine at position 508 of the CFTR amino acid sequence, and is commonly referred to as the F508del mutation. This mutation occurs in many of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease-causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Na⁺—K⁺-ATPase pump and Cl− channels expressed on the basolateral surface of the cell. Secondary active transport of chloride from the luminal side leads to the accumulation of intracellular chloride, which can then passively leave the cell via channels, resulting in a vectorial transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

A number of CFTR modulating compounds have recently been identified. However, compounds that can treat or reduce the severity of the cystic fibrosis and other CFTR mediated diseases, and particularly the more severe forms of these diseases, are still needed.

One aspect of the invention provides novel compounds, including compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing.

Formula (I) encompasses compounds falling within the following structure:

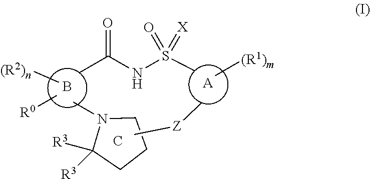

and includes tautomers of those compounds, deuterated derivatives of any of the compounds and tautomers, and pharmaceutically acceptable salts of any of the foregoing, wherein:
Ring A is a phenyl, indole, a 5-membered heteroaryl ring, or a 6-membered heteroaryl ring;
Ring B is a phenyl, pyridinyl, or pyrimidinyl ring;
X is O, NH, or an N(C₁-C₆ alkyl);
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R⁰ is R¹¹ or

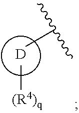

Ring D is a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, or a 3- to 8-membered cycloalkenyl;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
   k is 0, 1, 2, 3, 4, 5, or 6;
   each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
      each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
      each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
      each R^{a} is independently chosen from hydrogen and C₁-C₆ alkyl groups; and
   R⁷ is chosen from hydrogen, halogens, a cyano group, and C₃-C₁₀ cycloalkyl groups optionally substituted with one or more groups chosen from C₁-C₆ alkyl groups, C₁-C₆haloalkyl groups, and halogens;
q is 1, 2, 3 or 4;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl);
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
   r is 1, 2, 3, 4, 5, or 6;
   each L is independently chosen from C(R⁸)(R⁹) groups, —O—,

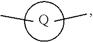

and —NR^{b}— groups, wherein a heteroatom in Z is not bonded to another heteroatom in Z, and wherein

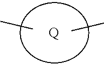

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).

Formula (I) includes compounds of Formula (II):

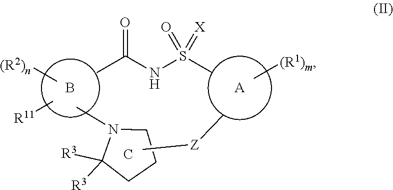

tautomers of those compounds, deuterated derivatives of any of the compounds and tautomers, and pharmaceutically acceptable salts of any of the foregoing wherein:
Ring A is a phenyl, indole, a 5-membered heteroaryl ring, or a 6-membered heteroaryl ring;
Ring B is a phenyl, pyridinyl, or pyrimidinyl ring;
X is O, NH, or an N(C₁-C₆ alkyl);
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl);
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
   r is 1, 2, 3, 4, 5, or 6;
   each L is independently chosen from C(R⁸)(R⁹) groups, —O—,

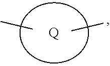

and —NR^{b}— groups, wherein a heteroatom in Z is not bonded to another heteroatom in Z, and wherein

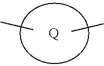

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).

Formula (I) also includes compounds of Formula (III):

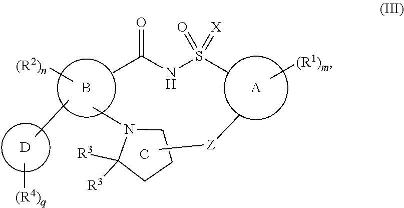

tautomers of those compounds, deuterated derivatives of any of the compounds and tautomers, and pharmaceutically acceptable salts of any of the foregoing, wherein:
Ring A is a phenyl, an indole, a 5-membered heteroaryl ring, or a 6-membered heteroaryl ring;
Ring B is a phenyl, pyridinyl, or pyrimidinyl ring;
Ring D is a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, or a 3- to 8-membered cycloalkenyl;
X is O, NH, or an N(C₁-C₆ alkyl);
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
   each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
   each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
   each R^{a} is independently chosen from hydrogen and C₁-C₆ alkyl groups; and
R⁷ is chosen from hydrogen, halogens, a cyano group, and C₃-C₁₀ cycloalkyl groups optionally substituted with one or more groups chosen from C₁-C₆ alkyl groups, C₁-C₆haloalkyl groups, and halogens;
q is 1, 2, 3 or 4; and
Z is a divalent linker of formula (L)ᵣ, wherein:
r is 1, 2, 3, 4, 5, or 6;
each L is independently chosen from C(R⁸)(R⁹) groups, —O—,

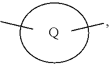

and —NR^{b}— groups, wherein a heteroatom in Z is not bonded to another heteroatom in Z, and wherein

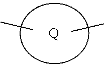

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);
provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

Another aspect of the invention provides pharmaceutical compositions comprising at least one compound chosen from the novel compounds disclosed herein, pharmaceutically acceptable salts thereof, and deuterated derivatives of any of the foregoing, and at least one pharmaceutically acceptable carrier, which compositions may further include at least one additional active pharmaceutical ingredient. Thus, another aspect of the invention provides methods of treating the CFTR-mediated disease cystic fibrosis comprising administering at least one of compound chosen from the novel compounds disclosed herein, pharmaceutically acceptable salts thereof, and deuterated derivatives of any of the foregoing, and at least one pharmaceutically acceptable carrier, optionally as part of a pharmaceutical composition comprising at least one additional component, to a subject in need thereof.

In certain embodiments, the pharmaceutical compositions of the invention comprise at least one compound chosen from compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing. In some embodiments, compositions comprising at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing may optionally further comprise (a) at least one compound chosen from tezacaftor and pharmaceutically acceptable salts and deuterated derivatives thereof; (b) at least one compound chosen from ivacaftor and pharmaceutically acceptable salts and deuterated derivatives thereof, such as D-ivacaftor; and/or (c) at least one compound chosen from lumacaftor and pharmaceutically acceptable salts and deuterated derivatives thereof.

Another aspect of the invention provides methods of treating the CFTR-mediated disease cystic fibrosis comprising administering to a patient in need thereof at least one compound chosen from the novel compounds disclosed herein, pharmaceutically acceptable salts thereof, and deuterated derivatives of any of the foregoing, and optionally further administering one or more additional CFTR modulating agents selected from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (tezacaftor), N-[2,4-bis(1,1-dimethylethyl)-5-hydroxyphenyl]-1,4-dihydro-4-oxoquinoline-3-carboxamide (ivacaftor) or N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (D-ivacaftor), and 3-(6-(1-(2,2-difluorobenzo[d][1,3]dioxol-5-yl)cyclopropane carboxamido)-3-methylpyridin-2-yl)benzoic acid (lumacaftor).

### Definitions

“Tezacaftor” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, which can be depicted with the following structure:

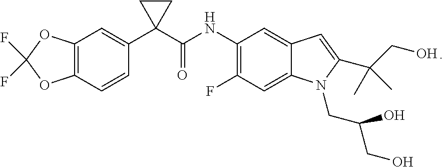 [SMILES: CC(C)(CO)c1cc2cc(NC(=O)C3(c4ccc5c(c4)OC(F)(F)O5)CC3)c(F)cc2n1C[C@@H](O)CO]

Tezacaftor may be in the form of a pharmaceutically acceptable salt. Tezacaftor and methods of making and using tezacaftor are disclosed in WO 2010/053471, WO 2011/119984, WO 2011/133751, WO 2011/133951, WO 2015/160787, and US 2009/0131492, each incorporated herein by reference.

“Ivacaftor” as used throughout this disclosure refers to N-(5-hydroxy-2,4-di-tert-butyl-phenyl)-4-oxo-1H-quinoline-3-carboxamide, which is depicted by the structure:

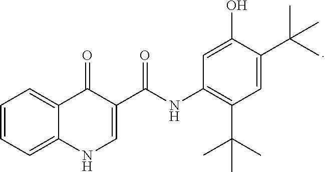 [SMILES: CC(C)(C)c1cc(C(C)(C)C)c(NC(=O)c2c[nH]c3ccccc3c2=O)cc1O]

Ivacaftor may also be in the form of a pharmaceutically acceptable salt. Ivacaftor and methods of making and using ivacaftor are disclosed in WO 2006/002421, WO 2007/079139, WO 2010/108162, and WO 2010/019239, each incorporated herein by reference.

In some embodiments, a deuterated derivative of ivacaftor (D-ivacaftor) is employed in the compositions and methods disclosed herein. A chemical name for D-ivacaftor is N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide, as depicted by the structure:

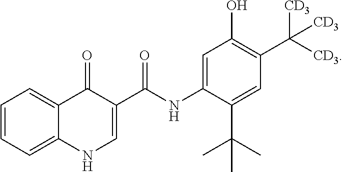 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

D-ivacaftor may be in the form of a pharmaceutically acceptable salt. D-ivacaftor and methods of making and using D-ivacaftor are disclosed in WO 2012/158885, WO 2014/078842, and U.S. Pat. No. 8,865,902, incorporated herein by reference.

“Lumacaftor” as used herein, refers to 3-(6-(1-(2,2-difluorobenzo[d][1,3]dioxol-5-yl)cyclopropanecarboxamido)-3-methylpyridin-2-yl)benzoic acid, which is depicted by the chemical structure:

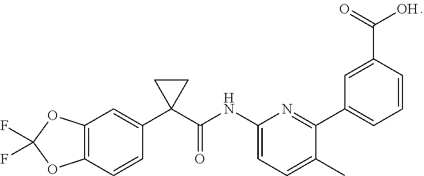 [SMILES: Cc1ccc(NC(=O)C2(c3ccc4c(c3)OC(F)(F)O4)CC2)nc1-c1cccc(C(=O)O)c1]

Lumacaftor may be in the form of a pharmaceutically acceptable salt. Lumacaftor and methods of making and using Lumacaftor are disclosed in WO 2007/056341, WO 2009/073757, and WO 2009/076142, incorporated herein by reference.

As used herein, the term “alkyl” refers to a saturated, branched or unbranched aliphatic hydrocarbon containing carbon atoms (such as, for example, 1, 2, 3, 4, 5, 6, 7, 8, 9, 10, 11, 12, 13, 14, 15, 16, 17, 18, 19, or 20 carbon atoms). Alkyl groups may be substituted or unsubstituted.

As used herein, the term “haloalkyl group” refers to an alkyl group substituted with one or more halogen atoms.

The term “alkoxy” as used herein refers to an alkyl or cycloalkyl covalently bonded to an oxygen atom. Alkoxy groups may be substituted or unsubstituted.

As used herein, the term “haloalkoxyl group” refers to an alkoxy group substituted with one or more halogen atoms.

As used herein, “cycloalkyl” refers to a cyclic, bicyclic, tricyclic, or polycyclic non-aromatic hydrocarbon groups having 3 to 12 carbons (such as, for example 3-10 carbons). “Cycloalkyl” groups encompass monocyclic, bicyclic, tricyclic, bridged, fused, and spiro rings, including mono spiro and dispiro rings. Non-limiting examples of cycloalkyl groups are cyclopropyl, cyclobutyl, cyclopentyl, cyclohexyl, adamantyl, norbomyl, and dispiro[2.0.2.1]heptane. Cycloalkyl groups may be substituted or unsubstituted.

The term “heteroaryl ring” as used herein refers to an aromatic ring comprising at least one ring atom that is a heteroatom, such as O, N, or S.

As used herein, the term “heterocyclyl ring” refers to a non-aromatic hydrocarbon containing 3 to 12 atoms in a ring (such as, for example 3-10 atoms) comprising at least one ring atom that is a heteroatom, such as O, N, or S. “Heterocyclyl” rings encompass monocyclic, bicyclic, tricyclic, polycyclic, bridged, fused, and spiro rings, including mono spiro and dispiro rings.

“Substituted,” whether preceded by the term “optionally” or not, indicates that at least one hydrogen of the “substituted” group is replaced by a substituent. Unless otherwise indicated, an “optionally substituted” group may have a suitable substituent at each substitutable position of the group, and when more than one position in any given structure may be substituted with more than one substituent chosen from a specified group, the substituent may be either the same or different at each position.

Examples of protecting groups for nitrogen include, for example, t-butyl carbamate (Boc), benzyl (Bn), para-methoxybenzyl (PMB), tetrahydropyranyl (THP), 9-fluorenylmethyl carbamate (Fmoc), benzyl carbamate (Cbz), methyl carbamate, ethyl carbamate, 2,2,2-trichloroethyl carbamate (Troc), 2-trimethylsilylethyl carbamate (Teoc), allyl carbamate (Aloc or Alloc), formamide, acetamide, benzamide, allylamine, trifluoroacetamide, triphenylmethylamine, benzylideneamine, and p-toluenesulfonamide. A comprehensive list of nitrogen protecting groups can be found in Wuts, P. G. M. “Greene's Protective Groups in Organic Synthesis: Fifth Edition,” 2014, John Wiley and Sons.

As used herein, “deuterated derivative(s)” refers to a compound having the same chemical structure as a reference compound, with one or more hydrogen atoms replaced by a deuterium atom.

As used herein, “CFTR” means cystic fibrosis transmembrane conductance regulator.

As used herein, the term “CFTR modulator” refers to a compound that increases the activity of CFTR. The increase in activity resulting from a CFTR modulator includes but is not limited to compounds that correct, potertiate, stabilize and/or amplify CFTR.

As used herein, the term “CFTR corrector” refers to a compound that facilitates the processing and trafficking of CFTR to increase the amount of CFTR at the cell surface. The novel compounds disclosed herein are CFTR correctors.

As used herein, the term “CFTR potentiator” refers to a compound that increases the channel activity of CFTR protein located at the cell surface, resulting in enhanced ion transport. Ivacaftor and D-ivacaftor disclosed herein are CFTR potentiators. It will be appreciated that when a description of a combination of compound selected from compounds of Formula (I), compounds of Formulae (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Ci), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, and other specified CFTR modulating agents is provided herein, reference to “ivacaftor or D-ivacaftor” in connection with the combination means that either ivacaftor or D-ivacaftor, but not both, is included in the combination.

As used herein, the term “active pharmaceutical ingredient” or “therapeutic agent” (“API”) refers to a biologically active compound.

The terms “patient” and “subject” are used interchangeably and refer to an animal including humans.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement in one or more symptoms of CF or lessening the severity of CF or one or more symptoms of CF in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

The terms “about” and “approximately”, when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent. The terms “about” and “approximately” may refer to an acceptable error for a particular value as determined by one of skill in the art, which depends in part on how the values is measured or determined. In some embodiments, the terms “about” and “approximately” mean within 20%, 15%, 10%, 5%, 4%, 3%, 2%, 1%, or 0.5% of a given value or range.

As used herein, the term “solvent” refers to any liquid in which the product is at least partially soluble (solubility of product >1 g/1).

As used herein, the term “room temperature” or “ambient temperature” means 15° C. to 30° C.

It will be appreciated that certain compounds of this invention may exist as separate stereoisomers or enantiomers and/or mixtures of those stereoisomers or enantiomers.

Certain compounds disclosed herein may exist as tautomers and both tautomeric forms are intended, even though only a single tautomeric structure is depicted. For example, a description of Compound A is understood to include its tautomer Compound B and vice versa, as well as mixtures thereof:

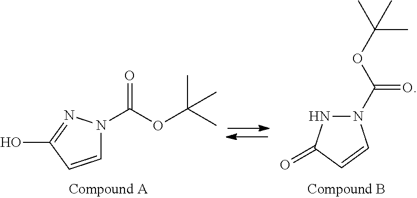

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance.

As used herein, the term “pharmaceutically acceptable salt” refers to a salt form of a compound of this disclosure wherein the salt is nontoxic. Pharmaceutically acceptable salts of the compounds of this disclosure include those derived from suitable inorganic and organic acids and bases. A “free base” form of a compound, for example, does not contain an ionically bonded salt.

The phrase “and pharmaceutically acceptable salts and deuterated derivatives thereof” is used interchangeably with “and pharmaceutically acceptable salts thereof and deuterated derivatives of any of the forgoing” in reference to one or more compounds or formulae of the invention. These phrases are intended to encompass pharmaceutically acceptable salts of any one of the referenced compounds, deuterated derivatives of any one of the referenced compounds, and pharmaceutically acceptable salts of those deuterated derivatives.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

### DETAILED DESCRIPTION OF EMBODIMENTS

In addition to compounds of Formula (II), tautomers thereof, deuterated derivatives of those compounds and tautomers, and pharmaceutically acceptable salts of any of the foregoing, the invention provides compounds of Formulae (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, tautomers thereof, deuterated derivatives of those compounds and tautomers, and pharmaceutically acceptable salts of any of the foregoing.

For example, in some embodiments, the compound of Formula (II) is a compound of Formula (II-Ai):

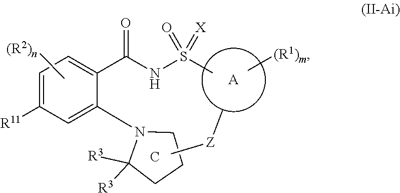

a tautomer thereof, a deuterated derivative of the compound or tautomer, or a pharmaceutically acceptable salt of any of the foregoing, wherein:
Ring A is a phenyl, indole, a 5-membered heteroaryl ring, or a 6-membered heteroaryl ring; —X is O, NH, or an N(C₁-C₆ alkyl);
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
r is 1, 2, 3, 4, 5, or 6;

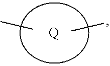

each L is independently chosen from C(R⁸)(R⁹) groups, —O—, and

NR^{b}— groups, wherein a heteroatom in Z is not bonded to another heteroatom in Z,

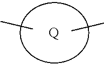

and wherein is a 5- or 6-membered heterocyclyl or a 5- or 6-membered heteroaryl, each of which is substituted with 0, 1, 2, 3, or 4 R¹⁰ groups; and

wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).

In some embodiments, the compound of Formula (II) is a compound of Formula (II-Aii), (II-Aiii), or (II-Aiv):

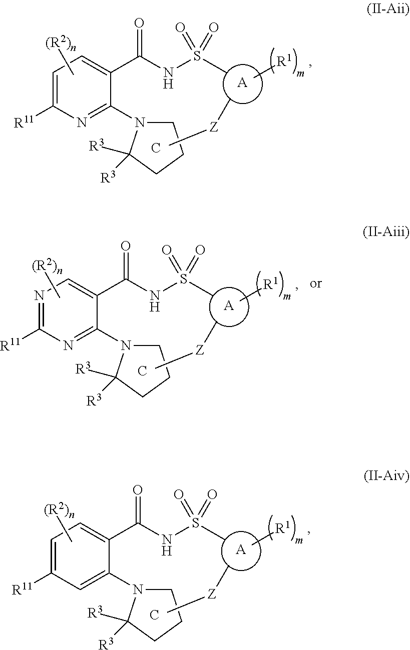

a tautomer thereof, a deuterated derivative of the compound or tautomer, or a pharmaceutically acceptable salt of any of the foregoing, wherein:
Ring A is a phenyl, indole, a 5-membered heteroaryl ring, or a 6-membered heteroaryl ring;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
r is 1, 2, 3, 4, 5, or 6;

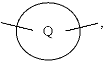

each L is independently chosen from C(R⁸)(R⁹) groups, —O—, and

NR^{b}— groups, wherein a heteroatom in Z is not bonded to another heteroatom in Z,

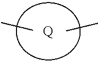

and wherein is a 5- or 6-membered heterocyclyl or a 5- or 6-membered heteroaryl, each of which is substituted with 0, 1, 2, 3, or 4 R¹⁰ groups; and

wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).

In some embodiments, the compound of Formula (II) is a compound of Formula (II-Av):

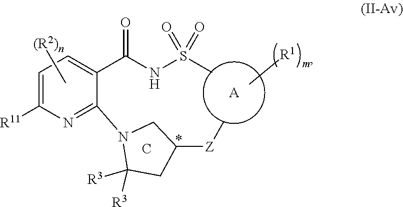

a tautomer thereof, a deuterated derivative of the compound or tautomer, or a pharmaceutically acceptable salt of any of the foregoing, wherein:
the carbon denoted by * has S-stereochemistry or R-stereochemistry;
Ring A is a phenyl, indole, a 5-membered heteroaryl ring, or a 6-membered heteroaryl ring;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
r is 1, 2, 3, 4, 5, or 6;

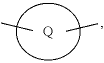

each L is independently chosen from C(R⁸)(R⁹) groups, —O—, and

NR^{b}— groups, wherein a heteroatom in Z is not bonded to another heteroatom in Z,

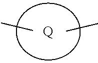

and wherein is a 5- or 6-membered heterocyclyl or a 5- or 6-membered heteroaryl, each of which is substituted with 0, 1, 2, 3, or 4 R¹⁰ groups; and

wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).

In some embodiments, the compound of Formula (II) is a compound of Formula (II-Avi):

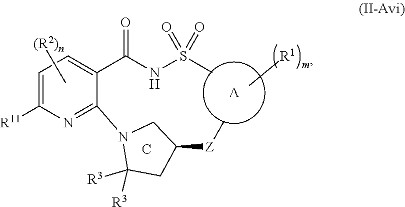

a tautomer thereof, a deuterated derivative of the compound or tautomer, or a pharmaceutically acceptable salt of any of the foregoing, wherein:
Ring A is a phenyl, indole, a 5-membered heteroaryl ring, or a 6-membered heteroaryl ring;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
r is 1, 2, 3, 4, 5, or 6;

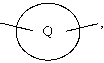

each L is independently chosen from C(R⁸)(R⁹) groups, —O—, and

NR^{b}— groups, wherein a heteroatom in Z is not bonded to another heteroatom in Z,

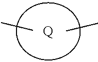

and wherein is a 5- or 6-membered heterocyclyl or a 5- or 6-membered heteroaryl, each of which is substituted with 0, 1, 2, 3, or 4 R¹⁰ groups; and

wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).

In some embodiments, the compound of Formula (II) is a compound of Formula (II-Bi), (II-Bii), (II-Biii), or (II-Biv):

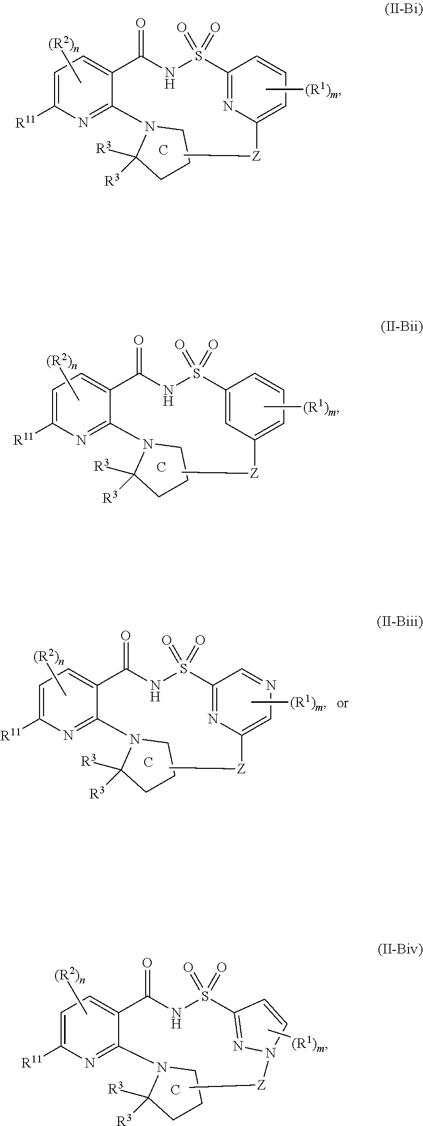

a tautomer thereof, a deuterated derivative of the compound or tautomer, or a pharmaceutically acceptable salt of any of the foregoing, wherein:
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
r is 1, 2, 3, 4, 5, or 6;

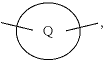

each L is independently chosen from C(R⁸)(R⁹) groups, —O—, and NR^{b}— groups, wherein a heteroatom in Z is not bonded to another heteroatom in Z,

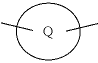

and wherein is a 5- or 6-membered heterocyclyl or a 5- or 6-membered heteroaryl, each of which is substituted with 0, 1, 2, 3, or 4 R¹⁰ groups; and

wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).

In some embodiments, the compound of Formula (II) is a compound of Formula (II-Bv):

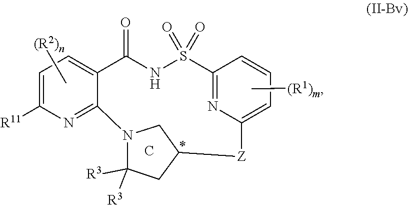

a tautomer thereof, a deuterated derivative of the compound or tautomer, or a pharmaceutically acceptable salt of any of the foregoing, wherein:
the carbon denoted by * has S-stereochemistry or R-stereochemistry;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
r is 1, 2, 3, 4, 5, or 6;

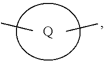

each L is independently chosen from C(R⁸)(R⁹) groups, —O—, and

NR^{b}— groups, wherein a heteroatom in Z is not bonded to another heteroatom in Z,

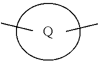

and wherein is a 5- or 6-membered heterocyclyl or a 5- or 6-membered heteroaryl, each of which is substituted with 0, 1, 2, 3, or 4 R¹⁰ groups; and

wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).

In some embodiments, the compound of Formula (II) is a compound of Formula (II-Bvi):

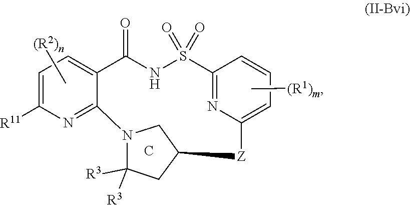

a tautomer thereof, a deuterated derivative of the compound or tautomer, or a pharmaceutically acceptable salt of any of the foregoing, wherein:
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
r is 1, 2, 3, 4, 5, or 6;

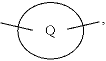

each L is independently chosen from C(R⁸)(R⁹) groups, —O—, and

NR^{b}— groups, wherein a heteroatom in Z is not bonded to another heteroatom in Z,

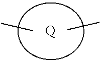

and wherein is a 5- or 6-membered heterocyclyl or a 5- or 6-membered heteroaryl, each of which is substituted with 0, 1, 2, 3, or 4 R¹⁰ groups; and

wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).

In some embodiments, the compound of Formula (II) is a compound of Formula (II-Ci), or (II-Civ):

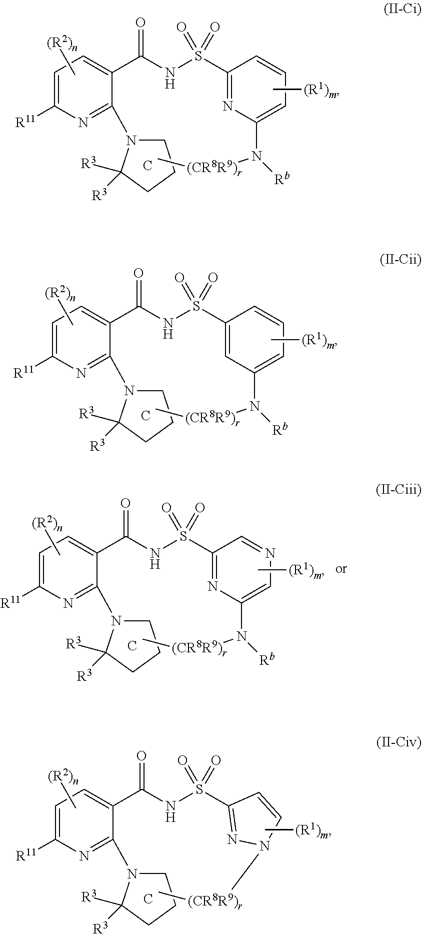

a tautomer thereof, a deuterated derivative of the compound or tautomer, or a pharmaceutically acceptable salt of any of the foregoing, wherein:
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
r is 1, 2, 3, 4, 5, or 6;
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).

In some embodiments, the compound of Formula (II) is a compound of Formula (II-Cv):

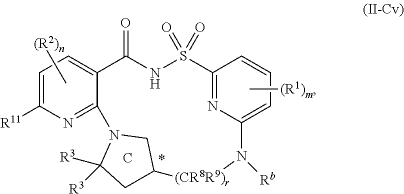

a tautomer thereof, a deuterated derivative of the compound or tautomer, or a pharmaceutically acceptable salt of any of the foregoing, wherein:
the carbon denoted by * has S-stereochemistry or R-stereochemistry;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
r is 1, 2, 3, 4, 5, or 6;
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).

In some embodiments, the compound of Formula (II) is a compound of Formula (II-Cvi):

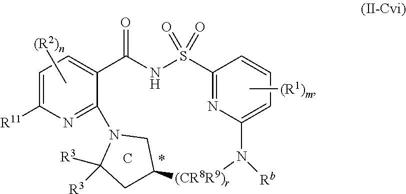

a tautomer thereof, a deuterated derivative of the compound or tautomer, or a pharmaceutically acceptable salt any of the foregoing, wherein:
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
r is 1, 2, 3, 4, 5, or 6;
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).

In some embodiments, the compound of Formula (II) is a compound other than Compounds 1, 43, 216, 223, 242, 251, 257, 258, 266, 270, and 271.

Also disclosed herein are compounds having a structural formula depicted in Table 3A, tautomers thereof, deuterated derivatives of those compounds and tautomers, and pharmaceutically acceptable salts of any of the foregoing.

In addition to compounds of Formula (III), tautomers thereof, deuterated derivatives of those compounds and tautomers, and pharmaceutically acceptable salts of any of the foregoing, the invention provides compounds of Formulae (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing.

For example, in some embodiments, the compound of Formula (III) is a compound of Formula (III-Ai), (III-Aii), or

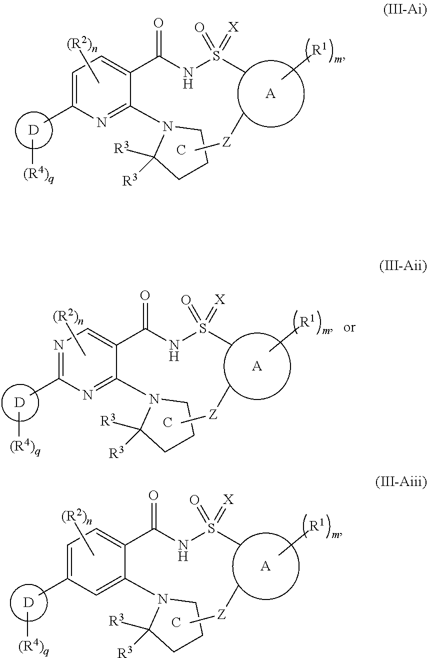

a tautomer thereof, a deuterated derivative of the compound or tautomer, or a pharmaceutically acceptable salt of any of the foregoing, wherein:
Ring A is a phenyl, an indole, a 5-membered heteroaryl ring, or a 6-membered heteroaryl ring;
Ring D is a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, or a 3- to 8-membered cycloalkenyl;
X is O, NH, or an N(C₁-C₆ alkyl);
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups;

and
R⁷ is chosen from hydrogen, halogens, a cyano group, and C₃-C₁₀ cycloalkyl groups optionally substituted with one or more groups chosen from C₁-C₆ alkyl groups, C₁-C₆haloalkyl groups, and halogens;
q is 1, 2, 3 or 4; and
Z is a divalent linker of formula (L)ᵣ, wherein:
r is 1, 2, 3, 4, 5, or 6;

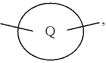

each L is independently chosen from C(R⁸)(R⁹) groups, —O—, and NR^{b}— groups, wherein a heteroatom in Z is not bonded to another heteroatom in

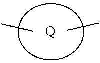

Z, and wherein is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);
provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

In some embodiments, the compound of Formula (III) is a compound of Formula (III-Aiv), (III-Av), or (III-Avi):

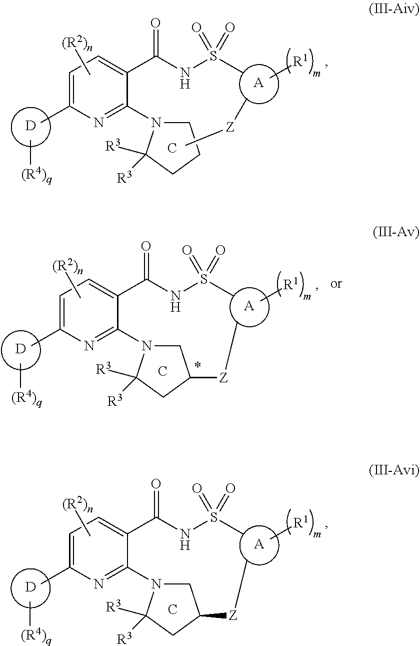

a tautomer thereof, a deuterated derivative of the compound or tautomer, or a pharmaceutically acceptable salt of any of the foregoing, wherein:
the carbon denoted by * has S-stereochemistry or R-stereochemistry;
Ring A is a phenyl, an indole, a 5-membered heteroaryl ring, or a 6-membered heteroaryl ring;
Ring D is a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, or a 3- to 8-membered cycloalkenyl;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups;

and
R⁷ is chosen from hydrogen, halogens, a cyano group, and C₃-C₁₀ cycloalkyl groups optionally substituted with one or more groups chosen from C₁-C₆ alkyl groups, C₁-C₆haloalkyl groups, and halogens;
q is 1, 2, 3 or 4; and
Z is a divalent linker of formula (L)ᵣ, wherein:
r is 1, 2, 3, 4, 5, or 6;

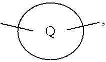

each L is independently chosen from C(R⁸)(R⁹) groups, —O—, and —NR^{b}— groups, wherein a heteroatom in Z is not bonded to another heteroatom in

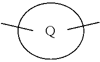

Z, and wherein is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);
provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

In some embodiments, the compound of Formula (III) is a compound of Formula (III-Avii) or (III-Aviii):

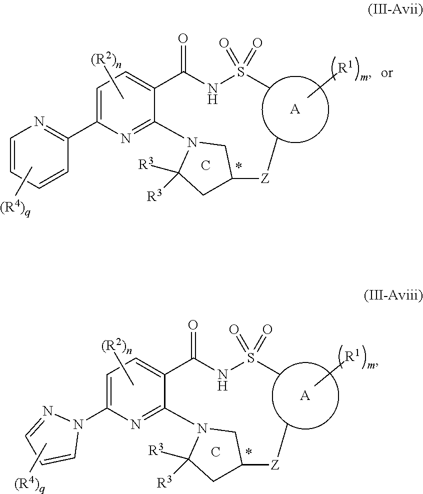

a tautomer thereof, a deuterated derivative of the compound or tautomer, or a pharmaceutically acceptable salt of any of the foregoing, wherein:
the carbon denoted by * has S-stereochemistry or R-stereochemistry;
Ring A is a phenyl, an indole, a 5-membered heteroaryl ring, or a 6-membered heteroaryl ring;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups;

and
R⁷ is chosen from hydrogen, halogens, a cyano group, and C₃-C₁₀ cycloalkyl groups optionally substituted with one or more groups chosen from C₁-C₆ alkyl groups, C₁-C₆haloalkyl groups, and halogens;
q is 1, 2, 3 or 4; and
Z is a divalent linker of formula (L)ᵣ, wherein:
r is 1, 2, 3, 4, 5, or 6;

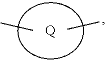

each L is independently chosen from C(R⁸)(R⁹) groups, —O—, and —NR^{b}— groups, wherein a heteroatom in Z is not bonded to another heteroatom in

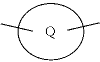

Z, and wherein is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);
provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

In some embodiments, the compound of Formula (III) is a compound of Formula (III-Bi), (III-Bii), (III-Biii), or (III-Biv):

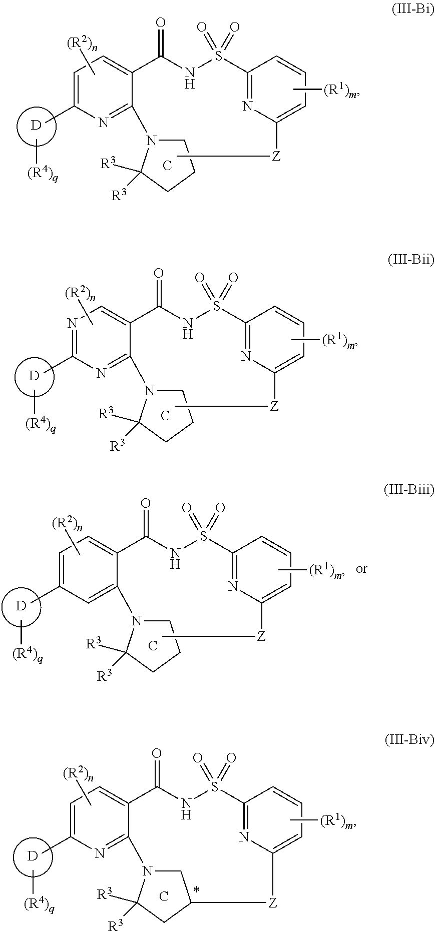

a tautomer thereof, a deuterated derivative of the compound or tautomer, or a pharmaceutically acceptable salt of any of the foregoing, wherein:
the carbon denoted by * has S-stereochemistry or R-stereochemistry;
Ring D is a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, or a 3- to 8-membered cycloalkenyl;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroary, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups;

and
R⁷ is chosen from hydrogen, halogens, a cyano group, and C₃-C₁₀ cycloalkyl groups optionally substituted with one or more groups chosen from C₁-C₆ alkyl groups, C₁-C₆haloalkyl groups, and halogens;
q is 1, 2, 3 or 4; and
Z is a divalent linker of formula (L)ᵣ, wherein:
r is 1, 2, 3, 4, 5, or 6;

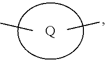

each L is independently chosen from C(R⁸)(R⁹) groups, —O—, and —NR^{b}— groups, wherein a heteroatom in Z is not bonded to another heteroatom in

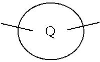

Z, and wherein is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);
provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

In some embodiments, the compound of Formula (III) is a compound of Formula (III-Bv) or (III-Bvi):

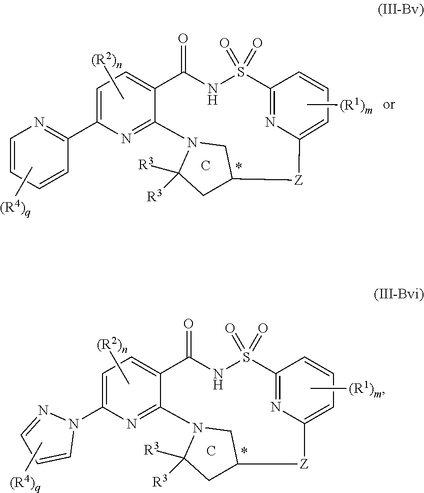

a tautomer thereof, a deuterated derivative of the compound or tautomer, or a pharmaceutically acceptable salt of any of the foregoing, wherein:
the carbon denoted by * has S-stereochemistry or R-stereochemistry;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroary, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups;

and
R⁷ is chosen from hydrogen, halogens, a cyano group, and C₃-C₁₀ cycloalkyl groups optionally substituted with one or more groups chosen from C₁-C₆ alkyl groups, C₁-C₆haloalkyl groups, and halogens;
q is 1, 2, 3 or 4; and
Z is a divalent linker of formula (L)ᵣ, wherein:
r is 1, 2, 3, 4, 5, or 6;

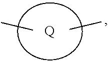

each L is independently chosen from C(R⁸)(R⁹) groups, —O—, and —NR^{b}— groups, wherein a heteroatom in Z is not bonded to another heteroatom in

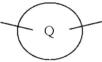

Z, and wherein is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);
provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

In some embodiments, the compound of Formula (III) is a compound of Formula (III-Ci), (III-Cii), (III-Ciii), or (III-Civ):

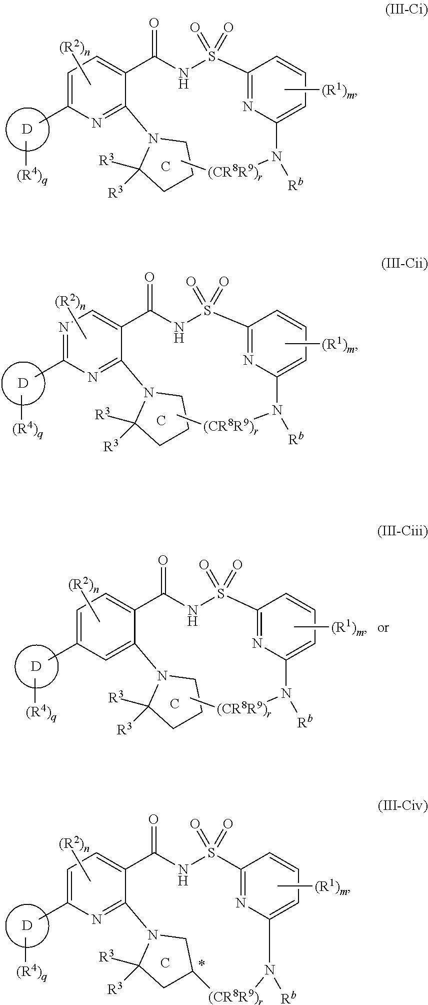

a tautomer thereof, a deuterated derivative of the compound or tautomer, or a pharmaceutically acceptable salt of any of the foregoing, wherein:
Ring D is a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, or a 3- to 8-membered cycloalkenyl;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups;

and
R⁷ is chosen from hydrogen, halogens, a cyano group, and C₃-C₁₀ cycloalkyl groups optionally substituted with one or more groups chosen from C₁-C₆ alkyl groups, C₁-C₆haloalkyl groups, and halogens;
q is 1, 2, 3 or 4;
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);
provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

In some embodiments, the compound of Formula (III) is a compound of Formula (III-Cv) or (III-Cvi):

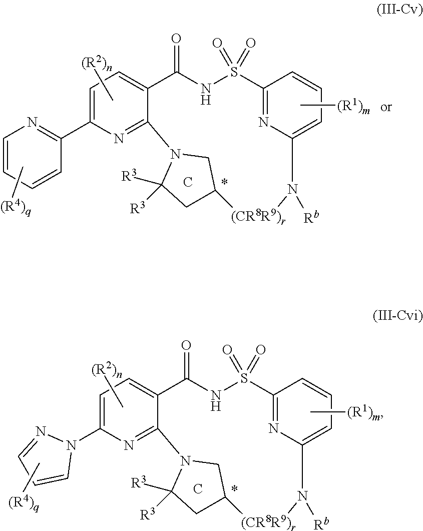

a tautomer thereof, a deuterated derivative of the compound or tautomer, or a pharmaceutically acceptable salt of any of the foregoing, wherein:
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups;

and
R⁷ is chosen from hydrogen, halogens, a cyano group, and C₃-C₁₀ cycloalkyl groups optionally substituted with one or more groups chosen from C₁-C₆ alkyl groups, C₁-C₆haloalkyl groups, and halogens;
q is 1, 2, 3 or 4;
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);
provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

Also disclosed herein are compounds having a structural formula depicted in Table 3B, tautomers thereof, deuterated derivatives of those compounds and tautomers, and pharmaceutically acceptable salts of any of the foregoing.

### Methods of Treatment

Any of the novel compounds disclosed herein, such as for example, compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, can act as a CFTR modulator, i.e., it modulates CFTR activity in the body. Individuals suffering from a mutation in the gene encoding CFTR may benefit from receiving a CFTR modulator. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in cystic fibrosis.

Thus, in some embodiments, the invention provides methods of treating, lessening the severity of, or symptomatically treating cystic fibrosis in a patient comprising administering to the patient an effective amount of any of the novel compounds disclosed herein, such as for example, compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing alone or in combination with another active ingredient, such as one or more CFTR modulating agents. In some embodiments, the one or more CFTR modulating agents are selected from ivacaftor, D-ivacaftor, lumacaftor, and tezacaftor. In some embodiments, the patient has an F508del/minimal function (MF) genotype, F508del/F508del genotype (homozygous for the F508del mutation), F508del/gating genotype, or F508del/residual function (RF) genotype. In some embodiments the patient is heterozygous and has one F508del mutation. In some embodiments the patient is homozygous for the N1303K mutation.

In some embodiments, 5 mg to 500 mg of a compound disclosed herein, a tautomer thereof, a deuterated derivatives of the compound and tautomer, or a pharmaceutically acceptable salt of any of the foregoing are administered daily.

In some embodiments, the patient is heterozygous and has an F508del mutation on one allele and a mutation on the other allele selected from Table 2:

TABLE 2
CFTR Mutations
Mutation

| | | | | |
|---|---|---|---|---|
| Q2X | L218X | Q525X | R792X | E1104X |
| S4X | Q220X | G542X | E822X | W1145X |
| W19X | Y275X | G550X | W882X | R1158X |
| G27X | C276X | Q552X | W846X | R1162X |
| Q39X | Q290X | R553X | Y849X | S1196X |
| W57X | G330X | E585X | R851X | W1204X |
| E60X | W401X | G673X | Q890X | L1254X |
| R75X | Q414X | Q685X | S912X | S1255X |
| L88X | S434X | R709X | Y913X | W1282X |
| E92X | S466X | K710X | Q1042X | Q1313X |
| Q98X | S489X | Q715X | W1089X | Q1330X |
| Y122X | Q493X | L732X | Y1092X | E1371X |
| E193X | W496X | R764X | W1098X | Q1382X |
| W216X | C524X | R785X | R1102X | Q1411X |
| 185 + 1G→T | 711 + 5G→A | 1717 − 8G→A | 2622 + 1G→A | 3121 − 1G→A |
| 296 + 1G→A | 712 − 1G→T | 1717 − 1G→A | 2790 − 1G→C | 3500 − 2A→G |
| 296 + 1G→T | 1248 + 1G→A | 1811 + 1G→C | 3040G→C | 3600 + 2insT |
| 405 + 1G→A | 1249 − 1G→A | 1811 + 1.6kbA→G | (G970R) | 3850 − 1G→A |
| 405 + 3A→C | 1341 + 1G→A | 1811 + 1643G→T | 3120G→A | 4005 + 1G→A |
| 406 − 1G→A | 1525 − 2A→G | 1812 − 1G→A | 3120 + 1G→A | 4374 + 1G→T |
| 621 + 1G→T | 1525 − 1G→A | 1898 + 1G→A | 3121 − 2A→G | |
| 711 + 1G→T | | 1898 + 1G→C | | |
| 182delT | 1078delT | 1677delTA | 2711delT | 3737delA |
| 306insA | 1119delA | 1782delA | 2732insA | 3791delC |
| 306delTAGA | 1138insG | 1824delA | 2869insG | 3821delT |
| 365-366insT | 1154insTC | 1833delT | 2896insAG | 3876delA |
| 394delTT | 1161delC | 2043delG | 2942insT | 3878delG |
| 442delA | 1213delT | 2143delT | 2957delT | 3905insT |
| 444delA | 1259insA | 2183AA→G | 3007delG | 4016insT |
| 457TAT→G | 1288insTA | 2184delA | 3028delA | 4021dupT |
| 541delC | 1343delG | 2184insA | 3171delC | 4022insT |
| 574delA | 1471delA | 2307insA | 3171insC | 4040delA |
| 663delT | 1497delGG | 2347delG | 3271delGG | 4279insA |
| 849delG | 1548delG | 2585delT | 3349insT | 4326delTC |
| 935delA | 1609del CA | 2594delGT | 3659delC | |
| CFTRdele1 | | CFTRdele16-17b | 1461ins4 | |
| CFTRdele2 | | CFTRdele17a, 17b | 1924del7 | |
| CFTRdele2, 3 | | CFTRdele17a-18 | 2055del9→A | |
| CFTRdele2-4 | | CFTRdele19 | 2105-2117del13insAGAAA | |
| CFTRdele3-10, 14b-16 | | CFTRdele19-21 | 2372del8 | |
| CFTRdele4-7 | | CFTRdele21 | 2721del11 | |
| CFTRdele4-11 | | CFTRdele22-24 | 2991del32 | |
| CFTR50kbdel | | CFTRdele22, 23 | 3667ins4 | |
| CFTRdup6b-10 | | 124del23bp | 4010del4 | |
| CFTRdele11 | | 602del14 | 4209TGTT→AA | |
| CFTRdele13, 14a | | 852del22 | | |
| CFTRdele14b-17b | | 991del5 | | |
| A46D | V520F | Y569D | N1303K | |
| G85E | A559T | L1065P | | |
| R347P | R560T | R1066C | | |
| L467P | R560S | L1077P | | |
| I507del | A561E | M1101K | | |

In some embodiments, the disclosure also is directed to methods of treatment using isotope-labelled compounds of the afore-mentioned compounds, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²F, ³⁵S, ¹⁸F and ³⁶Cl, respectively.

The isotope-labelled compounds and salts can be used in a number of beneficial ways. They can be suitable for medicaments and/or various types of assays, such as substrate tissue distribution assays. For example, tritium (³H)- and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (²H)-labelled ones. In some specific embodiments, the isotope-labelled compounds and salts are deuterium (²H)-labelled, wherein one or more hydrogen atoms therein have been replaced by deuterium. In chemical structures, deuterium is represented as “D.”

When discovering and developing therapeutic agents, the person skilled in the art attempts to optim/ze pharmacokinetic parameters while retaining desirable in vitro properties. It may be reasonable to assume that many compounds with poor pharmacokinetic profiles are susceptible to oxidative metabolism.

The deuterium (²H)-labelled compounds and salts can manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. For a further discussion, see S. L. Harbeson and R. D. Tung, Deuterium In Drug Discovery and Development, Ann. Rep. Med. Chem. 2011, 46, 403-417, which is incorporated herein by reference.

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Combination Therapies

One aspect disclosed herein provides methods of treating cystic fibrosis and other CFTR mediated diseases using any of the novel compounds disclosed herein, such as for example, compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (Ill-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, in combination with at least one additional active pharmaceutical ingredient.

In some embodiments, at least one additional active pharmaceutical ingredient is selected from mucolytic agents, bronchodilators, antibiotics, anti-infective agents, and anti-inflammatory agents.

In some embodiments, the additional therapeutic agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In some embodiments, the additional agent is a mucolyte. Exemplary mucolytes useful herein includes Pulmozyme®.

In some embodiments, the additional agent is a bronchodilator. Exemplary bronchodiltors include albuterol, metaprotenerol sulfate, pirbuterol acetate, salmeterol, or tetrabuline sulfate.

In some embodiments, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simavastatin.

In some embodiments, the additional agent is a nutritional agent. Exemplary nutritional agents include pancrelipase (pancreating enzyme replacement), including Pancrease®, Pancreacarb®, Ultrase®, or Creon®, Liprotomase® (formerly Trizytek®), Aquadeks®, or glutathione inhalation. In one embodiment, the additional nutritional agent is pancrelipase.

In some embodiments, at least one additional active pharmaceutical ingredient is selected from CFTR modulating agents. In some embodiments, the at least one additional active pharmaceutical ingredient is chosen from (a) tezacaftor and pharmaceutically acceptable salts thereof, and (b) ivacaftor or D-ivacaftor and pharmaceutically acceptable salts of ivacaftor or D-ivacaftor. Thus, in some embodiments, the combination therapies provided herein comprise (a) a compound selected from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing; (b) at least one compound selected from tezacaftor and pharmaceutically acceptable salts thereof; and (c) at least one compound selected from ivacaftor or D-ivacaftor, and pharmaceutically acceptable salts thereof. In some embodiments, the combination therapies provided herein comprise (a) at least one compound chosen from compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing; (b) at least one compound selected from tezacaftor and pharmaceutically acceptable salts thereof; and (c) at least one compound selected from ivacaftor or D-ivacaftor, and pharmaceutically acceptable salts thereof.

In some embodiments, at least one compound chosen from compounds of compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing is administered in combination with at least one compound chosen from tezacaftor and pharmaceutically acceptable salts thereof. In some embodiments, at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, is administered in combination with at least one compound chosen from ivacaftor and pharmaceutically acceptable salts thereof. In some embodiments, at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, is administered in combination with at least one compound chosen from D-ivacaftor and pharmaceutically acceptable salts thereof. In some embodiments, at least one compound chosen from compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, is administered in combination with tezacaftor or a pharmaceutically acceptable salt thereof and at least one compound chosen from ivacaftor and pharmaceutically acceptable salts thereof. In some embodiments, at least one compound chosen from compounds of Formula (I), compounds of Formulae (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, is administered in combination with at least one compound chosen from tezacaftor and pharmaceutically acceptable salts thereof and at least one compound chosen from D-ivacaftor and pharmaceutically acceptable salts thereof.

Each of the compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing independently can be administered once daily, twice daily, or three times daily. In some embodiments, at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-BA), (II-Bvi), (II-Ci), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, is administered once daily. In some embodiments, at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, is administered twice daily. In some embodiments, at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, and at least one compound chosen from tezacaftor and pharmaceutically acceptable salts thereof are administered once daily. In some embodiments, at least one compound chosen from compounds of compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, and at least one compound chosen from tezacaftor and pharmaceutically acceptable salts thereof are administered twice daily. In some embodiments, at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, and at least one compound chosen from ivacaftor or D-ivacaftor and pharmaceutically acceptable salts thereof are administered once daily. In some embodiments, at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, and at least one compound chosen from ivacaftor or D-ivacaftor and pharmaceutically acceptable salts thereof are administered twice daily.

In some embodiments, at least one compound chosen from compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, at least one compound chosen from tezacaftor and pharmaceutically acceptable salts thereof, and at least one compound chosen from ivacaftor or D-ivacaftor and pharmaceutically acceptable salts thereof are administered once daily. In some embodiments, at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, at least one compound chosen from ivacaftor or D-ivacaftor and pharmaceutically acceptable salts thereof, and at least one compound chosen from lumacaftor and pharmaceutically acceptable salts thereof, are administered once daily. In some embodiments, at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, at least one compound chosen from tezacaftor and pharmaceutically acceptable salts thereof, and at least one compound chosen from ivacaftor or D-ivacaftor and pharmaceutically acceptable salts thereof are administered twice daily. In some embodiments, at least one compound chosen from compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, at least one compound chosen from ivacaftor or D-ivacaftor and pharmaceutically acceptable salts thereof, and at least one compound chosen from lumacaftor and pharmaceutically acceptable salts thereof, are administered twice daily.

In some embodiments, at least one compound chosen from compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, and at least one compound chosen from tezacaftor and pharmaceutically acceptable salts thereof, are administered once daily and at least one compound chosen from D-ivacaftor and pharmaceutically acceptable salts thereof, are administered twice daily. In some embodiments, at least one compound chosen from compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, and at least one compound chosen from lumacaftor and pharmaceutically acceptable salts thereof, are administered once daily and at least one compound chosen from D-ivacaftor and pharmaceutically acceptable salts thereof, are administered twice daily.

Compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, tezacaftor, (ivacaftor or D-ivacaftor), and their pharmaceutically acceptable salts and deuterated derivatives thereof can be administered in a single pharmaceutical composition or separate pharmaceutical compositions. Such pharmaceutical compositions can be administered once daily or multiple times daily, such as twice daily. As used herein, the phrase that a given amount of API (e.g., tezacaftor, (ivacaftor or D-ivacaftor) or a pharmaceutically acceptable salt thereof) is administered once or twice daily or per day means that said given amount is administered per dosing once or twice daily.

In some embodiments, at least one compound chosen from compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, is administered in a first pharmaceutical composition; at least one compound chosen from tezacaftor and pharmaceutically acceptable salts thereof is administered in a second pharmaceutical composition; and at least one compound chosen from ivacaftor and pharmaceutically acceptable salts thereof is administered in a third pharmaceutical composition.

In some embodiments, at least one compound chosen from compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, is administered in a first pharmaceutical composition; at least one compound chosen from tezacaftor and pharmaceutically acceptable salts thereof is administered in a second pharmaceutical composition; at least one compound chosen from D-ivacaftor and pharmaceutically acceptable salts thereof is administered in a third pharmaceutical composition.

In some embodiments, at least one compound chosen from compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, is administered in a first pharmaceutical composition; at least one compound chosen from ivacftor or D-ivacaftor and pharmaceutically acceptable salts thereof is administered in a second pharmaceutical composition; at least one compound chosen from lumacaftor and pharmaceutically acceptable salts thereof is administered in a third pharmaceutical composition.

In some embodiments, at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, is administered in a first pharmaceutical composition; and at least one compound chosen from tezacaftor and pharmaceutically acceptable salts thereof and at least one compound chosen from ivacaftor or D-ivacaftor, and pharmaceutically acceptable salts thereof are administered in a second pharmaceutical composition. In some embodiments, the second pharmaceutical composition comprises a half of a daily dose of said at least one compound chosen from ivacaftor or D-ivcaftor, and pharmaceutically acceptable salts thereof, and the other half of said at least one compound chosen from ivacaftor or D-ivacaftor, and pharmaceutically acceptable salts thereof is administered in a third pharmaceutical composition.

In some embodiments, at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing; at least one compound chosen from tezacaftor and pharmaceutically acceptable salts thereof and at least one compound chosen from ivacaftor or D-ivacaftor, and pharmaceutically acceptable salts thereof are administered in a first pharmaceutical composition. In some embodiments, the first pharmaceutical composition is administered to the patient twice daily. In some embodiments the first pharmaceutical composition is administered once daily. In some embodiments the first pharmaceutical composition is administered once daily and a second composition comprising only ivacaftor is administered once daily.

Any suitable pharmaceutical compositions can be used for compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tezacaftor, ivacaftor, D-ivacaftor, lumacaftor and tautomers thereof, deuterated derivatives of those compounds and tautomers, and pharmaceutically acceptable salts of any of the foregoing. Some exemplary pharmaceutical compositions for tezacaftor and its pharmaceutically acceptable salts can be found in WO 2011/119984 and WO 2014/014841, incorporated herein by reference. Some exemplary pharmaceutical compositions for ivacaftor and its pharmaceutically acceptable salts can be found in WO 2007/134279, WO 2010/019239, WO 2011/019413, WO 2012/027731, and WO 2013/130669, and some exemplary pharmaceutical compositions for D-ivacaftor and its pharmaceutically acceptable salts can be found in U.S. Pat. Nos. 8,865,902, 9,181,192, 9,512,079, WO 2017/053455, and WO 2018/080591, all of which are incorporated herein by reference. Some exemplary pharmaceutical compositions for lumacaftor and its pharmaceutically acceptable salts can be found in WO 2010/037066, WO 2011/127421, and WO 2014/071122, incorporated herein by reference.

### Pharmaceutical Compositions

Another aspect of the invention provides a pharmaceutical composition comprising at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, and at least one pharmaceutically acceptable carrier.

In some embodiments, the invention provides pharmaceutical compositions comprising at least one compound chosen from compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises at least one compound chosen from compounds of Formula (I), compounds of Formulae (II), (II-Ai), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, and at least two additional active pharmaceutical ingredients, one of which is a CFTR corrector and one of which is a CFTR potentiator.

In some embodiments, the invention provides a pharmaceutical composition comprising (a) at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, (b) at least one compound chosen from tezacaftor and pharmaceutically acceptable salts thereof, and (c) at least one pharmaceutically acceptable carrier.

In some embodiments, the disclosure provides a pharmaceutical composition comprising (a) at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, (b) at least one compound chosen from ivacaftor, D-ivacaftor, and pharmaceutically acceptable salts thereof, and (c) at least one pharmaceutically acceptable carrier.

In some embodiments, the disclosure provides a pharmaceutical composition comprising (a) at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, (b) at least one compound chosen from tezacaftor and pharmaceutically acceptable salts thereof, (c) at least one compound chosen from ivacaftor and pharmaceutically acceptable salts thereof, and (d) at least one pharmaceutically acceptable carrier.

In some embodiments, the disclosure provides a pharmaceutical composition comprising (a) at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, (b) at least one compound chosen from tezacaftor and pharmaceutically acceptable salts thereof, (c) at least one compound chosen from D-ivacaftor and pharmaceutically acceptable salts thereof, and (d) at least one pharmaceutically acceptable carrier.

In some embodiments, the disclosure provides a pharmaceutical composition comprising (a) at least one compound chosen from compounds of Formula (I), compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), Compounds 1-298, compounds of Formulae (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), Compounds 299 to 397, Compounds 398-436, tautomers thereof, deuterated derivatives of those compounds and tautomers, and pharmaceutically acceptable salts of any of the foregoing, (b) at least one compound chosen from ivacaftor or D-ivacaftor and pharmaceutically acceptable salts thereof, (c) at least one compound chosen from lumacaftor and pharmaceutically acceptable salts thereof, and (d) at least one pharmaceutically acceptable carrier.

Any pharmaceutical composition disclosed herein may comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, lubricants.

The pharmaceutical compositions described herein are useful for treating cystic fibrosis and other CFTR mediated diseases.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

Additional embodiments include:

1. A compound of Formula (I):

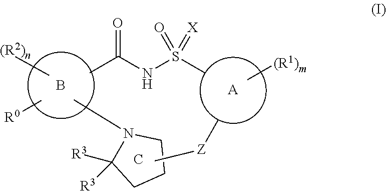

or a tautomer thereof, a deuterated derivative of the compound or tautomer, or a pharmaceutically acceptable salt of any of the foregoing, wherein:
Ring A is a phenyl, indole, a 5-membered heteroaryl ring, or a 6-membered heteroaryl ring;
Ring B is a phenyl, pyridinyl, or pyrimidinyl ring;
X is O, NH, or an N(C₁-C₆ alkyl);
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;

R⁰ is R¹¹ or

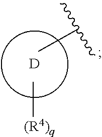

Ring D is a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, or a 3- to 8-membered cycloalkenyl;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
   k is 0, 1, 2, 3, 4, 5, or 6;
   each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
      each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
      each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
      each R^{a} is independently chosen from hydrogen and C₁-C₆ alkyl groups; and
   R⁷ is chosen from hydrogen, halogens, a cyano group, and C₃-C₁₀ cycloalkyl groups optionally substituted with one or more groups chosen from C₁-C₆ alkyl groups, C₁-C₆haloalkyl groups, and halogens;
q is 1, 2, 3 or 4;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl);
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
   r is 1, 2, 3, 4, 5, or 6;
   each L is independently chosen from C(R⁸)(R⁹) groups, —O—,

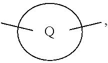

and —NR^{b}— groups, wherein a heteroatom in Z is not bonded to another heteroatom in Z, and wherein

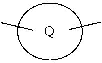

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
   each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).
      2. The compound of embodiment 1, wherein the compound of Formula (I) is a compound of Formula (II):

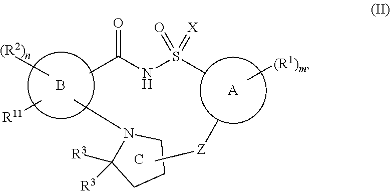

a tautomer thereof, a deuterated derivative of the compound or tautomer, or a pharmaceutically acceptable salt of any of the foregoing, wherein:
Ring A is a phenyl, indole, a 5-membered heteroaryl ring, or a 6-membered heteroaryl ring;
Ring B is a phenyl, pyridinyl, or pyrimidinyl ring;
X is O, NH, or an N(C₁-C₆ alkyl);
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl);
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring, or two R³ are joined to form a C₃-C₆ cycloaklyl ring;
Z is a divalent linker of formula (L)ᵣ, wherein:
   r is 1, 2, 3, 4, 5, or 6;
   each L is independently chosen from C(R⁸)(R⁹) groups, —O—,

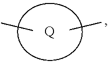

and —NR^{b}— groups, wherein a heteroatom in Z is not bonded to another heteroatom in Z, and wherein

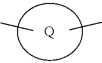

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).
   3. The compound of embodiment 2, wherein the compound of Formula (II) is a compound of Formula (II-Ai):

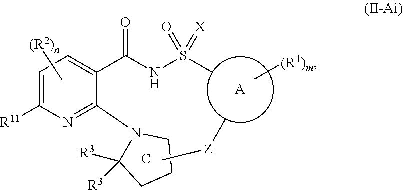

a tautomer thereof, a deuterated derivative of the compound or tautomer, or a pharmaceutically acceptable salt of any of the foregoing, wherein:
Ring A is a phenyl, indole, a 5-membered heteroaryl ring, or a 6-membered heteroaryl ring;
X is O, NH, or an N(C₁-C₆ alkyl);
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
   r is 1, 2, 3, 4, 5, or 6;
   each L is independently chosen from C(R⁸)(R⁹) groups, —O—,

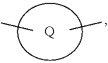

and —NR^{b}— groups, wherein a heteroatom in Z is not bonded to another heteroatom in Z, and wherein

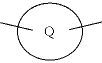

is a 5- or 6-membered heterocyclyl or a 5- or 6-membered heteroaryl, each of which is substituted with 0, 1, 2, 3, or 4 R¹⁰ groups; and

wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).
   4. The compound of embodiment 2, wherein the compound of Formula (II) is a compound of Formula (II-Aii), (II-Aiii), or (II-Aiv):

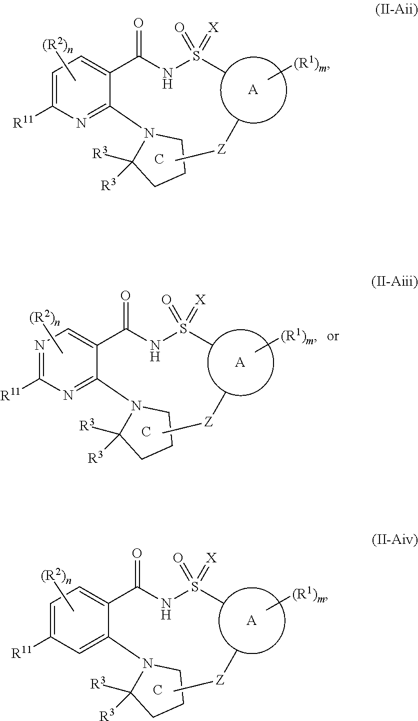

a tautomer thereof, a deuterated derivative of the compound or tautomer, or a pharmaceutically acceptable salt of any of the foregoing, wherein:
Ring A is a phenyl, indole, a 5-membered heteroaryl ring, or a 6-membered heteroaryl ring;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
   r is 1, 2, 3, 4, 5, or 6;
   each L is independently chosen from C(R⁸)(R⁹) groups, —O—,

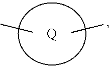

and —NR^{b}— groups, wherein a heteroatom in Z is not bonded to another heteroatom in Z, and wherein

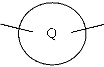

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).
   5. The compound of embodiment 2, wherein the compound of Formula (II) is a compound of Formula (II-Av):

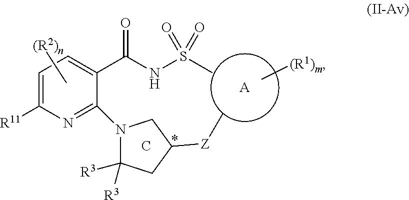

a tautomer thereof, a deuterated derivative of the compound or tautomer, or a pharmaceutically acceptable salt of any of the foregoing, wherein:
the carbon denoted by * has S-stereochemistry or R-stereochemistry;
Ring A is a phenyl, indole, a 5-membered heteroaryl ring, or a 6-membered heteroaryl ring;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-COM, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
   r is 1, 2, 3, 4, 5, or 6;
   each L is independently chosen from C(R⁸)(R⁹) groups, —O—,

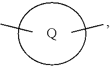

and —NR^{b}— groups, wherein a heteroatom in Z is not bonded to another heteroatom in Z, and wherein

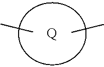

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).
   6. The compound of embodiment 2, wherein the compound of Formula (II) is a compound of Formula (II-Avi):

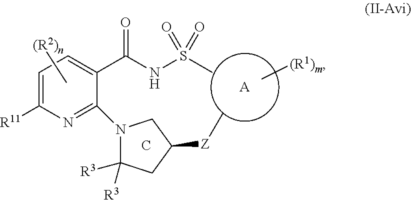

a tautomer thereof, a deuterated derivative of the compound or tautomer, or a pharmaceutically acceptable salt of any of the foregoing, wherein:
Ring A is a phenyl, indole, a 5-membered heteroaryl ring, or a 6-membered heteroaryl ring;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
   r is 1, 2, 3, 4, 5, or 6;
   each L is independently chosen from C(R⁸)(R⁹) groups, —O—,

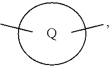

and —NR^{b}— groups, wherein a heteroatom in Z is not bonded to another heteroatom in Z, and wherein

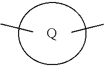

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄⁻C₉ heterocyclyl is optionally substituted with —(C₁-C₆ alkyl)-O(C₁-C₆ alkyl) or —CO₂(C₁-C₆ alkyl).
   7. The compound of embodiment 2, wherein the compound of Formula (II) is a compound of Formula (II-Bi), (II-Bii), (II-Biii), or (II-Biv):

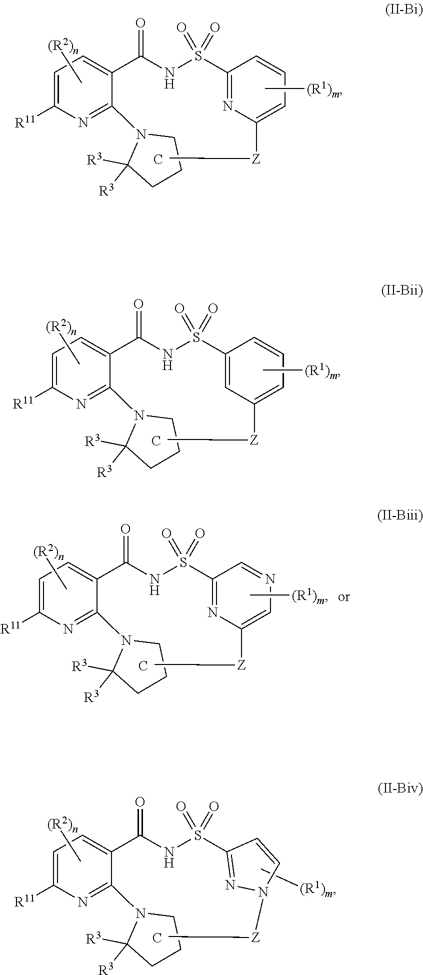

a tautomer thereof, a deuterated derivative of the compound or tautomer, or a pharmaceutically acceptable salt of any of the foregoing, wherein:
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
   r is 1, 2, 3, 4, 5, or 6;
   each L is independently chosen from C(R⁸)(R⁹) groups, —O—,

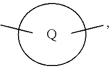

and —NR^{b}— groups, wherein a heteroatom in Z is not bonded to another heteroatom in Z, and wherein

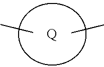

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).
   8. The compound of embodiment 2, wherein the compound of Formula (II) is a compound of Formula (II-Bv):

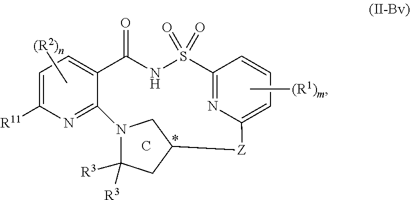

a tautomer thereof, a deuterated derivative of the compound or tautomer, or a pharmaceutically acceptable salt of any of the foregoing, wherein:
the carbon denoted by * has S-stereochemistry or R-stereochemistry;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
   r is 1, 2, 3, 4, 5, or 6;
   each L is independently chosen from C(R⁸)(R⁹) groups, —O—,

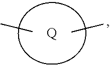

and —NR^{b}— groups, wherein a heteroatom in Z is not bonded to another heteroatom in Z, and wherein

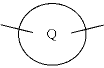

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).
   9. The compound of embodiment 1, wherein the compound of Formula (II) is a compound of Formula (II-Bvi):

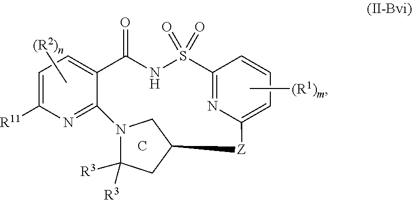

a tautomer thereof, a deuterated derivative of the compound or tautomer, or a pharmaceutically acceptable salt of any of the foregoing, wherein:
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
   r is 1, 2, 3, 4, 5, or 6;
   each L is independently chosen from C(R⁸)(R⁹) groups, —O—,

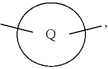

and —NR^{b}— groups, wherein a heteroatom in Z is not bonded to another heteroatom in Z, and wherein

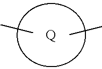

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).
   10. The compound of embodiment 2, wherein the compound of Formula (II) is a compound of Formula (II-Ci, (II-Ciii), or (II-Civ):

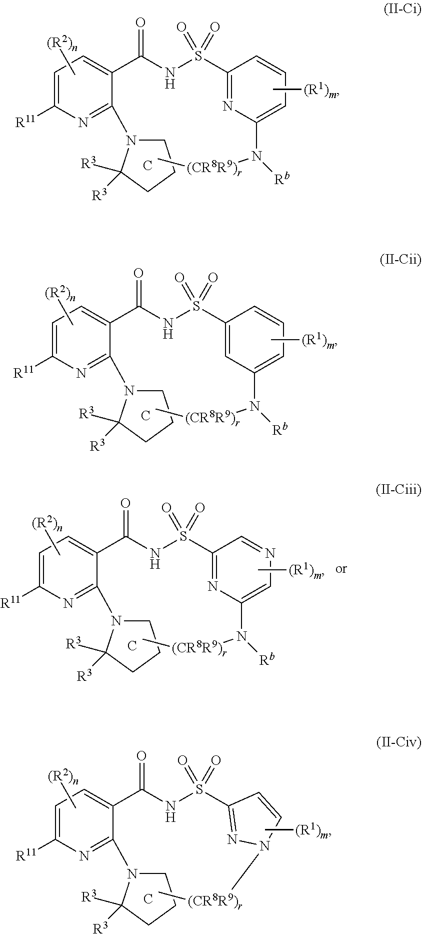

a tautomer thereof, a deuterated derivative of the compound or tautomer, or a pharmaceutically acceptable salt of any of the foregoing, wherein:
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
r is 1, 2, 3, 4, 5, or 6;
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
   or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).
   11. The compound of embodiment 2, wherein the compound of Formula (II) is a compound of Formula (II-Cv):

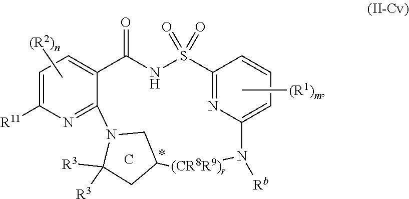

a tautomer thereof, a deuterated derivative of the compound or tautomer, or a pharmaceutically acceptable salt of any of the foregoing, wherein:
the carbon denoted by * has S-stereochemistry or R-stereochemistry;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
r is 1, 2, 3, 4, 5, or 6;
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).
   12. The compound of embodiment 2, wherein the compound of Formula (II) is a compound of Formula (II-Cvi):

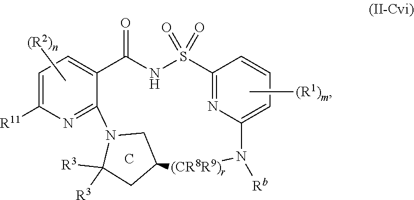

a tautomer thereof, a deuterated derivative of the compound or tautomer, or a pharmaceutically acceptable salt of any of the foregoing, wherein:
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
r is 1, 2, 3, 4, 5, or 6;
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).
   13. The compound, salt, or deuterated derivative of embodiment 2 or 3, wherein X is O.
   14. The compound, tautomer, deuterated derivative, or pharmaceutically acceptable salt of any one of embodiments 2 to 6, wherein Ring A is selected from phenyl, pyridine, pyrizine, and pyrazole.
   15. The compound, tautomer, deuterated derivative, or pharmaceutically acceptable salt of any one of embodiments 2 to 7, wherein:
Ring A is a phenyl; and
one R¹ and one R^{b}, together with the atoms to which they are attached, form a pyrrole or a pyridine.
   16. The compound, tautomer, deuterated derivative, or pharmaceutically acceptable salt of embodiment 2 or 4, wherein Ring B is a pyridinyl ring.
   17. The compound, tautomer, deuterated derivative, or pharmaceutically acceptable salt of any one of embodiments 2 to 12, wherein R¹ is selected from hydrogen and hydroxyl.
   18. The compound, tautomer, deuterated derivative, or pharmaceutically acceptable salt of any one of embodiments 2 to 12, wherein R³ is a C₁-C₆ alkyl substituted with a phenyl.

19. The compound, tautomer, deuterated derivative, or pharmaceutically acceptable salt of any one of embodiments 2 to 12, wherein R³ is benzyl.

20. The compound, tautomer, deuterated derivative, or pharmaceutically acceptable salt of any one of embodiments 2 to 12, wherein R^{b} is selected from H, CH₃, phenyl, and isobutyl.

21. The compound, tautomer, deuterated derivative, or pharmaceutically acceptable salt of any one of embodiments 2 to 12, wherein each C(R⁸)(R⁹) group is independently chosen from —CH₂—, —CO—,

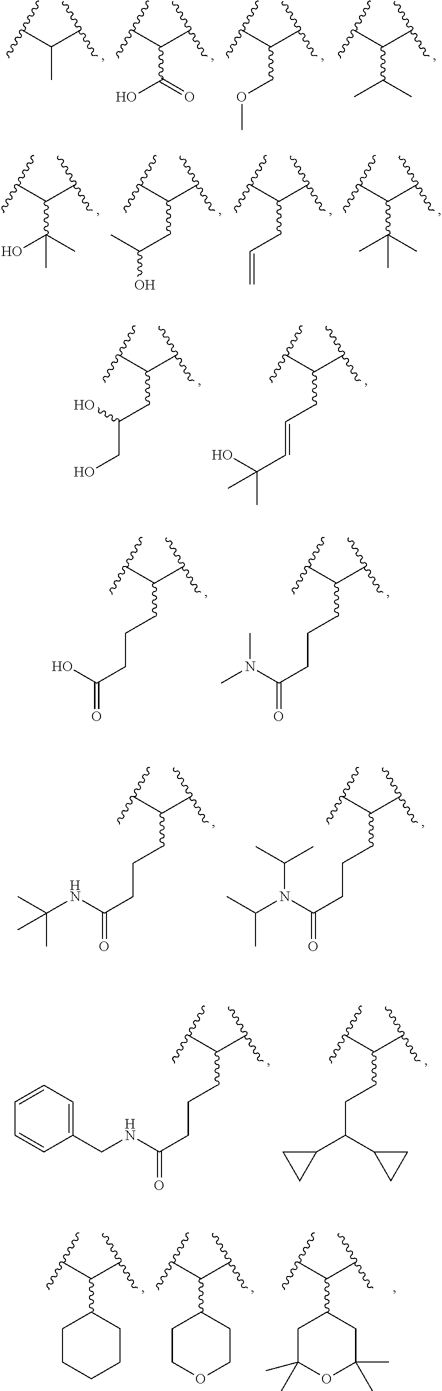

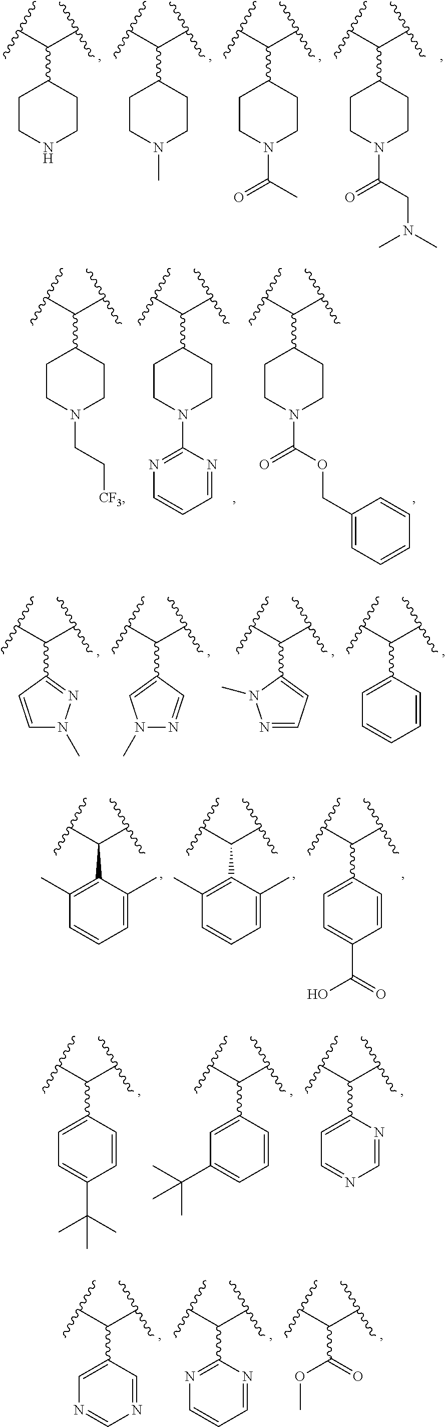

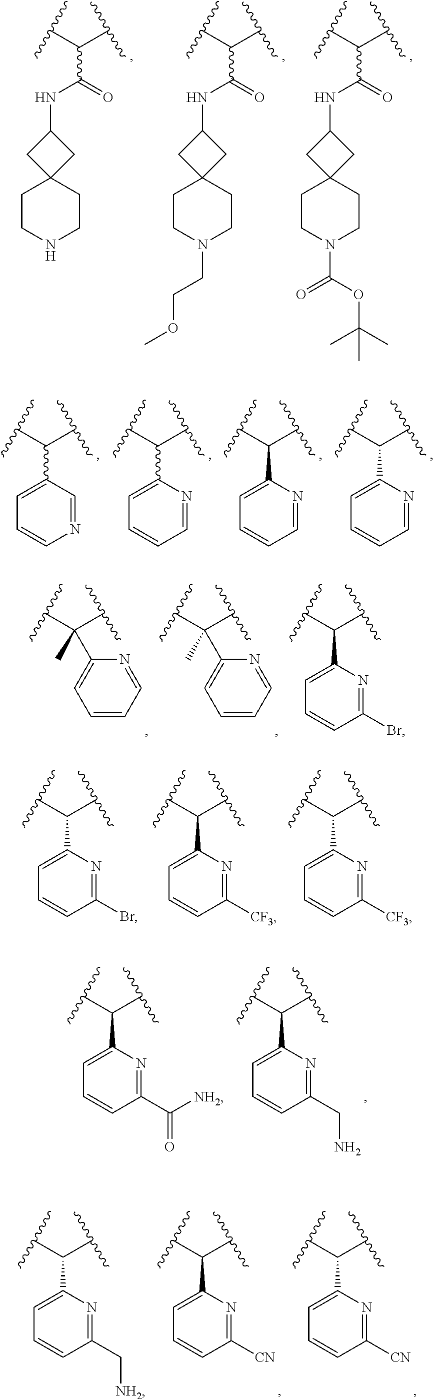

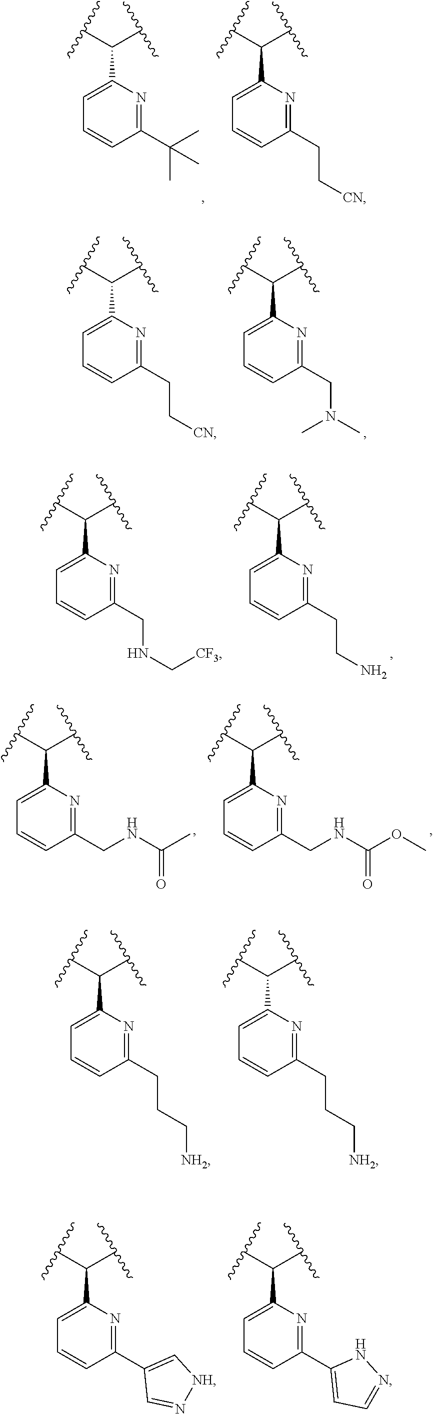

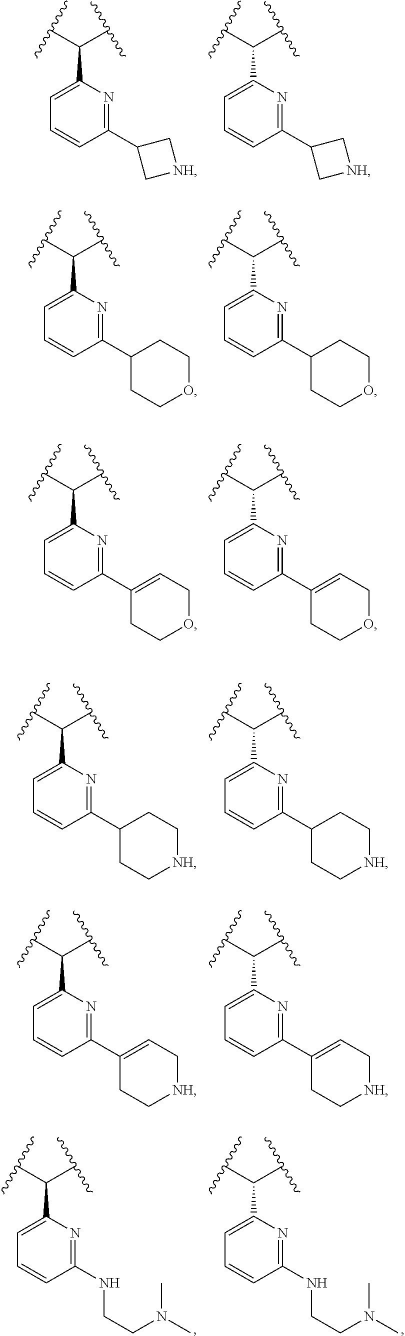

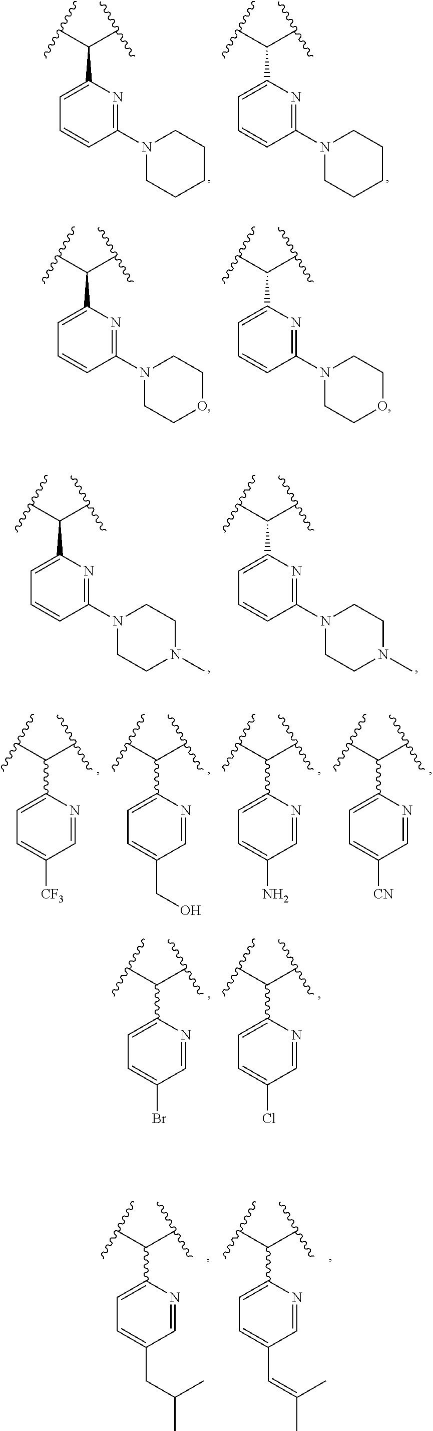

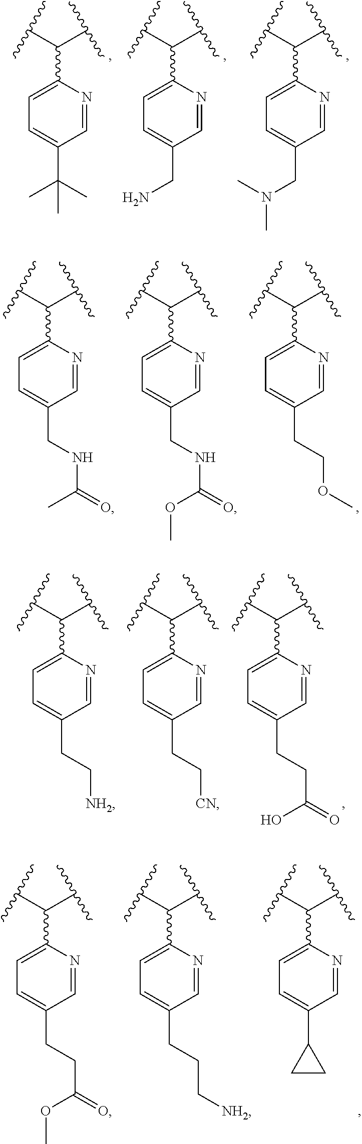

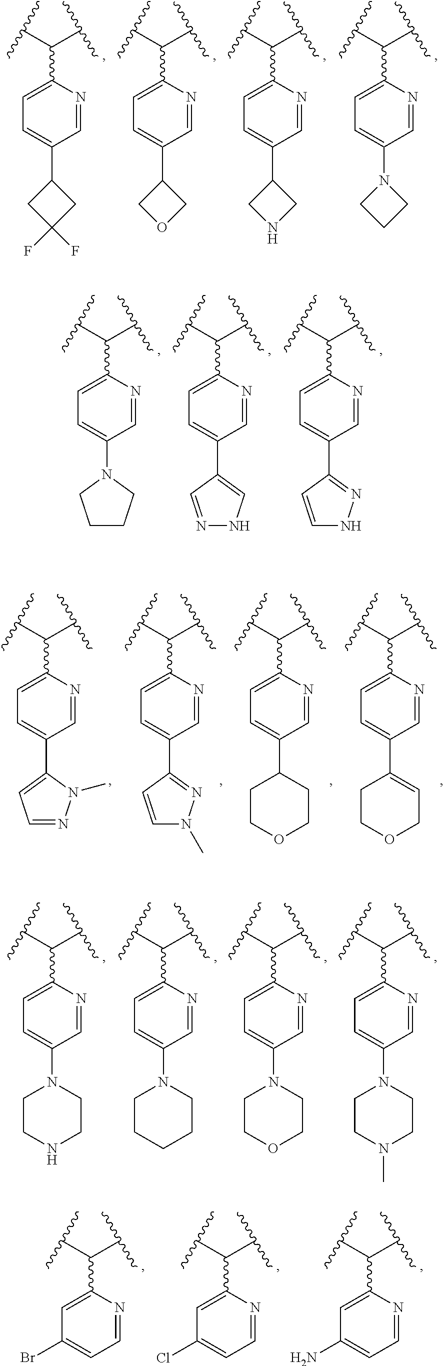

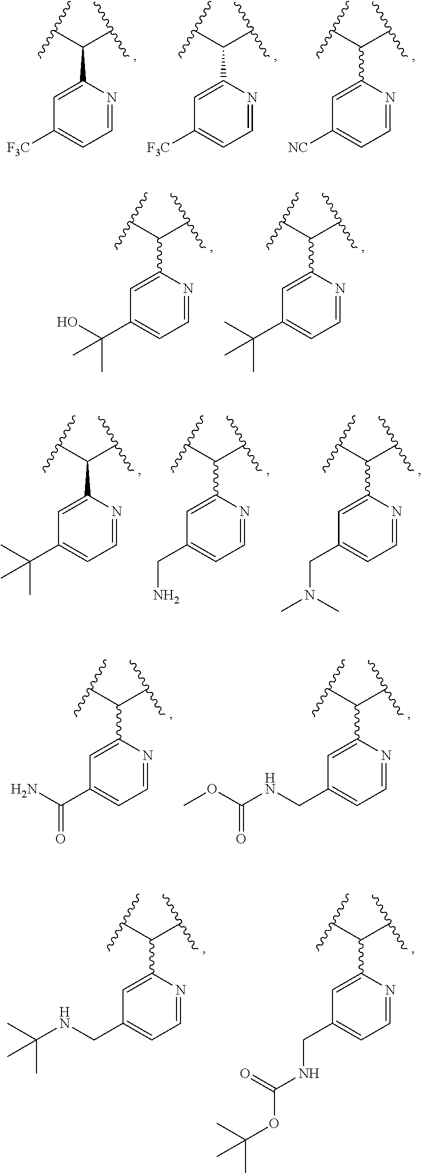

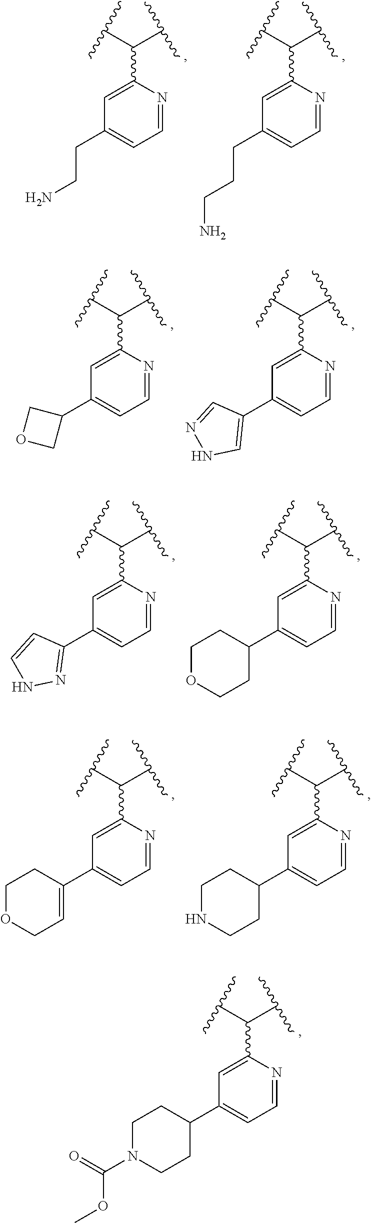

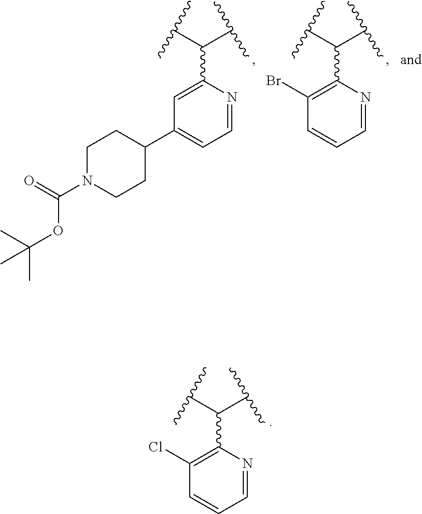

22. The compound, tautomer, deuterated derivative, or pharmaceutically acceptable salt of any one of embodiments 2 to 9, wherein

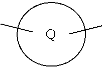

is

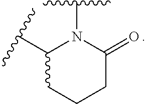 [SMILES: *C1CCCC(=O)N1*]

23. The compound, tautomer, deuterated derivative, or pharmaceutically acceptable salt of any one of embodiments 2 to 12, wherein R¹¹ is chosen from hydrogen, halogen, cyano,

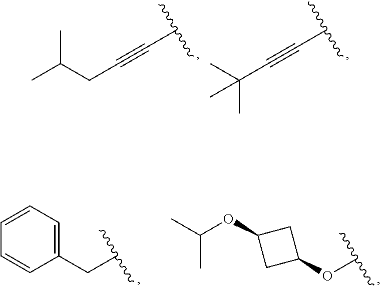

and t-Bu.
24. The compound, tautomer, deuterated derivative, or pharmaceutically acceptable salt of any one of embodiments 2 to 12, wherein R¹¹ is t-Bu.
25. The compound, tautomer, deuterated derivative, or pharmaceutically acceptable salt of any one of embodiments 2 to 12, wherein:
one R² and R¹¹, together with the atoms to which they are attached, form a phenyl, tetrahydropyran, or cyclohexyl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups; and
each R² is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group.
   26. A compound selected from Compounds 1-298 (Table 3A), tautomers thereof, deuterated derivatives of the compounds and tautomers, and pharmaceutically acceptable salts of any of the foregoing.
   27. A pharmaceutical composition comprising a compound, tautomer, deuterated derivative, or pharmaceutically acceptable salt of any one of embodiments 1-26 and a pharmaceutically acceptable carrier.
   28. The pharmaceutical composition of embodiment 27, further comprising one or more additional therapeutic agent(s).
   29. The pharmaceutical composition of embodiment 28, wherein the one or more additional therapeutic agents are selected from tezacaftor, ivacaftor, D-ivacaftor, lumacaftor, and pharmaceutically acceptable salts thereof.
   30. The pharmaceutical composition of embodiment 29, wherein the composition comprises tezacaftor and ivacaftor.
   31. The pharmaceutical composition of embodiment 29 wherein the composition comprises tezacaftor and D-ivacaftor.
   32. A pharmaceutical composition comprising:
(a) at least one compound, tautomer, deuterated derivative, or pharmaceutically acceptable salt according to any one of embodiments 1-26;
(b) at least one pharmaceutically acceptable carrier; and optionally one or more of:
(c) (i) a compound chosen from tezacaftor:

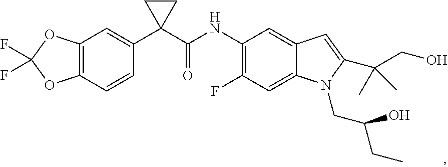 [SMILES: CC[C@H](O)Cn1c(C(C)(C)CO)cc2cc(NC(=O)C3(c4ccc5c(c4)OC(F)(F)O5)CC3)c(F)cc21]

and pharmaceutically acceptable salts and deuterated derivatives thereof; and

(ii) a compound chosen from ivacaftor

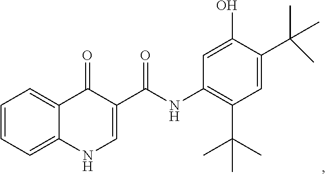 [SMILES: CC(C)(C)c1cc(C(C)(C)C)c(NC(=O)c2c[nH]c3ccccc3c2=O)cc1O]

### D-ivacaftor

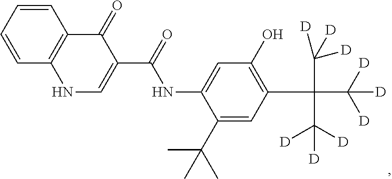 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

and pharmaceutically acceptable salts and deuterated derivatives thereof.
33. A method of treating cystic fibrosis comprising administering to a patient in need thereof a compound, tautomer, deuterated derivative, or pharmaceutically acceptable salt of any one of embodiments 1-26 or a pharmaceutical composition according to any one of embodiments 27-32.
34. The method of embodiment 33, further comprising administering to the patient one or more additional therapeutic agent(s) prior to, concurrent with, or subsequent to the compound or the pharmaceutical composition.
35. The method of embodiment 33, wherein the one or more additional therapeutic agent(s) comprise(s) a compound selected from tezacaftor, ivacaftor, D-ivacaftor, lumacaftor, and pharmaceutically acceptable salts thereof.
36. The method of embodiment 35, wherein the one or more additional therapeutic agent(s) comprise(s) tezacaftor and ivacaftor.
37. The method of embodiment 35, wherein the one or more additional therapeutic agent(s) comprise(s) tezacaftor and D-ivacaftor.
38. The compound, salt, or deuterated derivative of any one of embodiments 1-26 or the pharmaceutical composition according to any one of embodiments 27-32 for use in the treatment of cystic fibrosis.
39. The compound, tautomer, deuterated derivative, or pharmaceutically acceptable salt of any one of embodiments 1-26 or the pharmaceutical composition according to any one of embodiments 27-32 for use in the manufacture of a medicament for the treatment of cystic fibrosis.
40. The compound of embodiment 1, wherein the compound of Formula (I) is a compound of Formula (III):

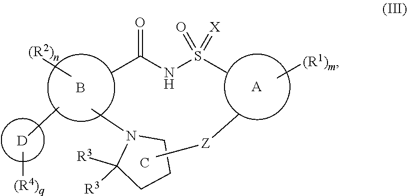

a tautomer thereof, a deuterated derivative of the compound or tautomer, or a pharmaceutically acceptable salt of any of the foregoing,
wherein:
Ring A is a phenyl, an indole, a 5-membered heteroaryl ring, or a 6-membered heteroaryl ring;
Ring B is a phenyl, pyridinyl, or pyrimidinyl ring;
Ring D is a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, or a 3- to 8-membered cycloalkenyl;
X is O, NH, or an N(C₁-C₆ alkyl);
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloalkyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
   k is 0, 1, 2, 3, 4, 5, or 6;
each Y is independently chosen from C(R⁵)(R⁶) groups, —O—, and —NR^{a}— groups,
   wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups; and
R⁷ is chosen from hydrogen, halogens, a cyano group, and C₃-C₁₀ cycloalkyl groups optionally substituted with one or more groups chosen from C₁-C₆ alkyl groups, C₁-C₆ haloalkyl groups, and halogens;
q is 1, 2, 3 or 4; and
Z is a divalent linker of formula (L)ᵣ, wherein:
   r is 1, 2, 3, 4, 5, or 6;
   each L is independently chosen from C(R⁸)(R⁹) groups, —O—,

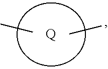

and —NR^{b}— groups, wherein a heteroatom in Z is not bonded to another heteroatom in Z, and wherein

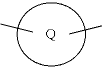

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl), or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);
provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.
   41. The compound of embodiment 40, wherein the Compound of Formula (III) is a compound of Formula (III-Ai), (III-Aii), or (III-Aiii):

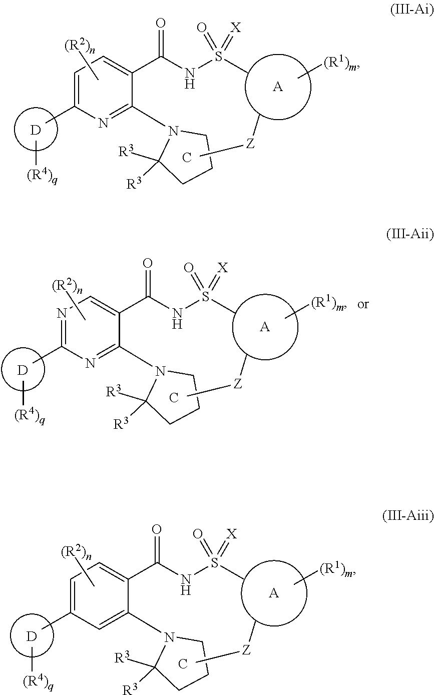

a tautomer thereof, a deuterated derivative of the compound or tautomer, or a pharmaceutically acceptable salt of any of the foregoing, wherein:
Ring A is a phenyl, an indole, a 5-membered heteroaryl ring, or a 6-membered heteroaryl ring;
Ring D is a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, or a 3- to 8-membered cycloalkenyl;
X is O, NH, or an N(C₁-C₆ alkyl);
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
   k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups; and
R⁷ is chosen from hydrogen, halogens, a cyano group, and C₃-C₁₀ cycloalkyl groups optionally substituted with one or more groups chosen from C₁-C₆ alkyl groups, C₁-C₆ haloalkyl groups, and halogens;
q is 1, 2, 3 or 4; and
Z is a divalent linker of formula (L)ᵣ, wherein:
   r is 1, 2, 3, 4, 5, or 6;
   each L is independently chosen from C(R⁸)(R⁹) groups, —O—,

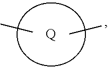

and —NR^{b}— groups, wherein a heteroatom in Z is not bonded to another heteroatom in Z, and wherein

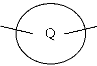

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);

provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

42. The compound of embodiment 40, wherein the compound of Formula (III) is a compound of Formula (III-Aiv), (III-Av), or (III-Avi):

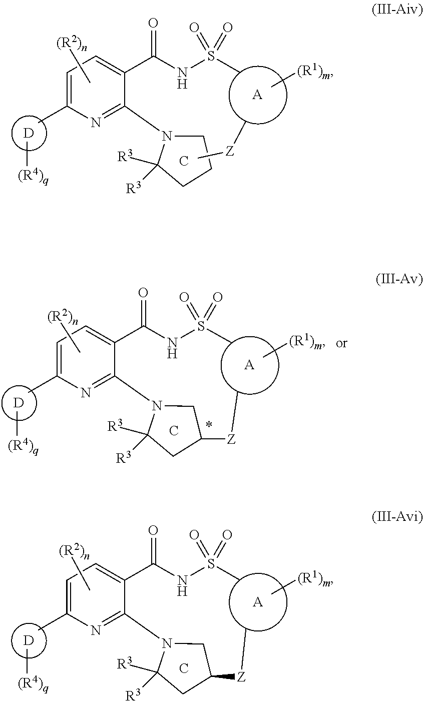

a tautomer thereof, a deuterated derivative of the compound or tautomer, or a pharmaceutically acceptable salt of any of the foregoing, wherein:
the carbon denoted by * has S-stereochemistry or R-stereochemistry;
Ring A is a phenyl, an indole, a 5-membered heteroaryl ring, or a 6-membered heteroaryl ring;
Ring D is a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, or a 3- to 8-membered cycloalkenyl;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
   k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups; and
R⁷ is chosen from hydrogen, halogens, a cyano group, and C₃-C₁₀ cycloalkyl groups optionally substituted with one or more groups chosen from C₁-C₆ alkyl groups, C₁-C₆ haloalkyl groups, and halogens;
q is 1, 2, 3 or 4; and
Z is a divalent linker of formula (L)ᵣ, wherein:
   r is 1, 2, 3, 4, 5, or 6;
   each L is independently chosen from C(R⁸)(R⁹) groups, —O—,

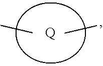

and —NR^{b}— groups, wherein a heteroatom in Z is not bonded to another heteroatom in Z, and wherein

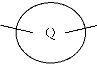

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;

each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);

provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

43. The compound of embodiment 40, wherein the compound of Formula (III) is a compound of Formula (III-Avii) or (III-Aviii):

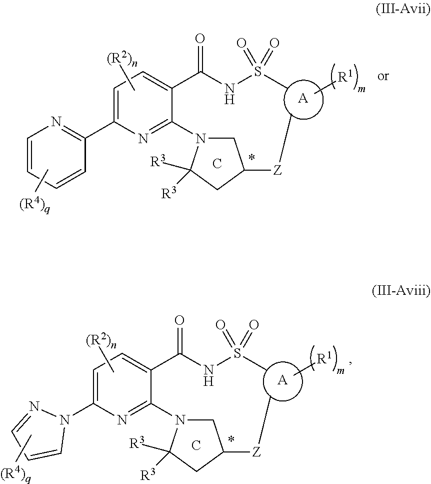

a tautomer thereof, a deuterated derivative of the compound or tautomer, or a pharmaceutically acceptable salt of any of the foregoing, wherein:
the carbon denoted by * has S-stereochemistry or R-stereochemistry;
Ring A is a phenyl, an indole, a 5-membered heteroaryl ring, or a 6-membered heteroaryl ring;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
   k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups; and
R⁷ is chosen from hydrogen, halogens, a cyano group, and C₃-C₁₀ cycloalkyl groups optionally substituted with one or more groups chosen from C₁-C₆ alkyl groups, C₁-C₆ haloalkyl groups, and halogens;
q is 1, 2, 3 or 4; and
Z is a divalent linker of formula (L)ᵣ, wherein:
   r is 1, 2, 3, 4, 5, or 6;
   each L is independently chosen from C(R⁸)(R⁹) groups, —O—,

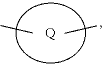

and —NR^{b}— groups, wherein a heteroatom in Z is not bonded to another heteroatom in Z, and wherein

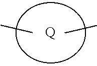

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);

provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

44. The compound of embodiment 40, wherein the compound of Formula (III) is a compound of Formula (III-Bi), (III-Bii), (III-Biii), or (III-Biv):

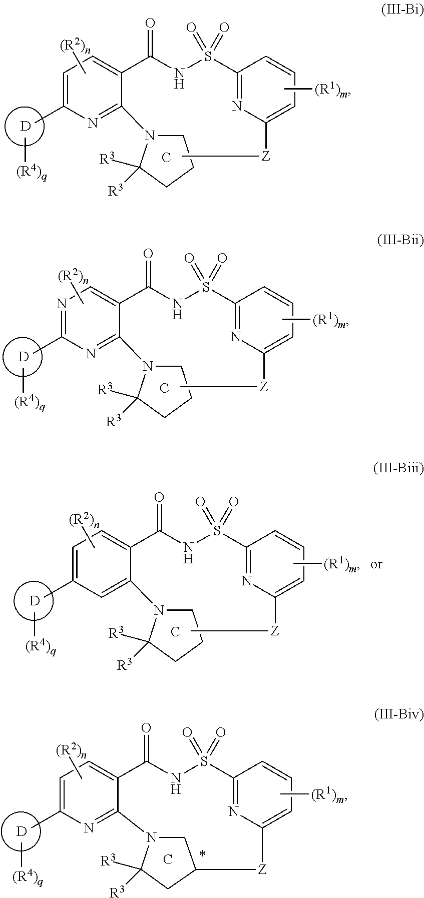

a tautomer thereof, a deuterated derivative of the compound or tautomer, or a pharmaceutically acceptable salt of any of the foregoing, wherein:
the carbon denoted by * has S-stereochemistry or R-stereochemistry;
Ring D is a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, or a 3- to 8-membered cycloalkenyl;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
   k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups; and
R⁷ is chosen from hydrogen, halogens, a cyano group, and C₃-C₁₀ cycloalkyl groups optionally substituted with one or more groups chosen from C₁-C₆ alkyl groups, C₁-C₆ haloalkyl groups, and halogens;
q is 1, 2, 3 or 4; and
Z is a divalent linker of formula (L)ᵣ, wherein:
   r is 1, 2, 3, 4, 5, or 6;
   each L is independently chosen from C(R⁸)(R⁹) groups, —O—,

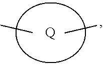

and —NR^{b}— groups, wherein a heteroatom in Z is not bonded to another heteroatom in Z, and wherein

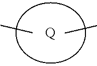

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);

provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

45. The compound of embodiment 40, wherein the compound of Formula (III) is a compound of Formula (III-Bv) or (III-Bvi):

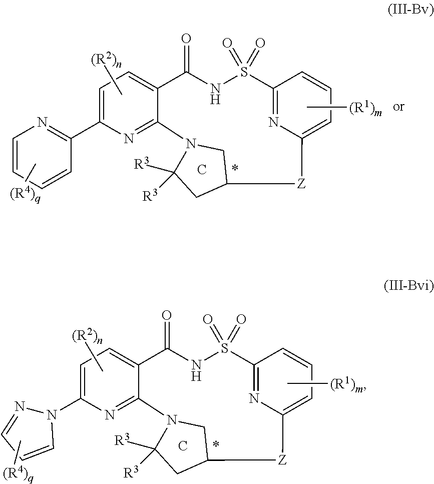

a tautomer thereof, a deuterated derivative of the compound or tautomer, or a pharmaceutically acceptable salt of any of the foregoing, wherein:
the carbon denoted by * has S-stereochemistry or R-stereochemistry;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
   k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups; and
R⁷ is chosen from hydrogen, halogens, a cyano group, and C₃-C₁₀ cycloalkyl groups optionally substituted with one or more groups chosen from C₁-C₆ alkyl groups, C₁-C₆ haloalkyl groups, and halogens;
q is 1, 2, 3 or 4; and
Z is a divalent linker of formula (L)ᵣ, wherein:
   r is 1, 2, 3, 4, 5, or 6;
   each L is independently chosen from C(R⁸)(R⁹) groups, —O—,

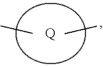

and —NR^{b}— groups, wherein a heteroatom in Z is not bonded to another heteroatom in Z, and wherein

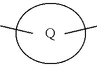

is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;

each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);

provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or C₁ alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

46. The compound of embodiment 40, wherein the compound of Formula (III) is a compound of Formula (III-Ci), (III-Cii), (III-Ciii), or (III-Civ):

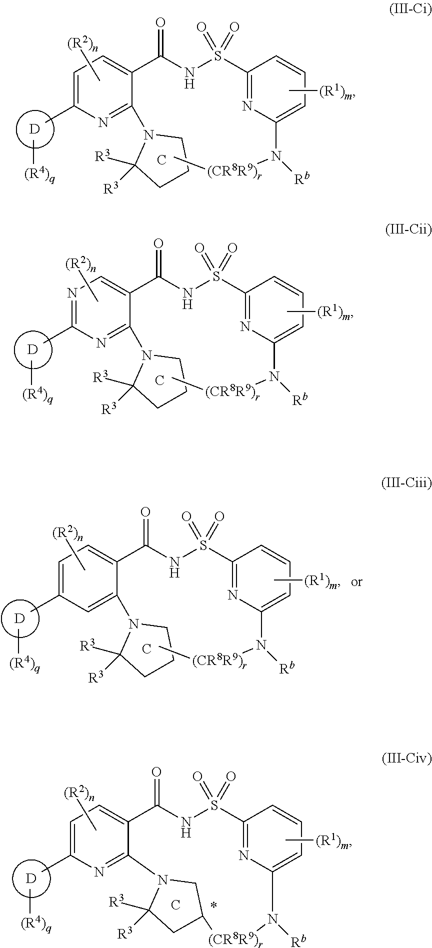

a tautomer thereof, a deuterated derivative of the compound or tautomer, or a pharmaceutically acceptable salt of any of the foregoing, wherein:
Ring D is a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, or a 3- to 8-membered cycloalkenyl;
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
   k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups; and
R⁷ is chosen from hydrogen, halogens, a cyano group, and C₃-C₁₀ cycloalkyl groups optionally substituted with one or more groups chosen from C₁-C₆ alkyl groups, C₁-C₆ haloalkyl groups, and halogens;
q is 1, 2, 3 or 4;
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);

provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

47. The compound of embodiment 40, wherein the compound of Formula (III) is a compound of Formula (III-Cv) or (III-Cvi):

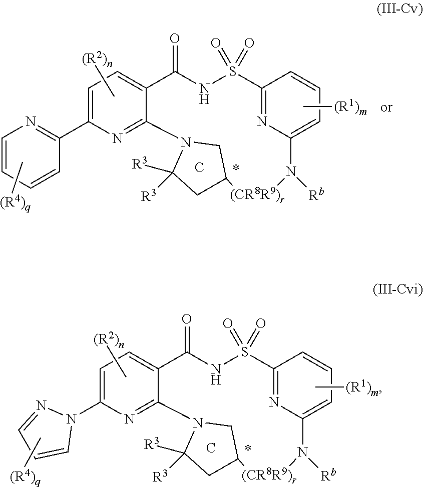

a tautomer thereof, a deuterated derivative of the compound or tautomer, or a pharmaceutically acceptable salt of any of the foregoing, wherein:
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group, or optionally two R², together with the atoms they are attached to, form a phenyl or a 6-membered heteroaryl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups;
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
   k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups; and
R⁷ is chosen from hydrogen, halogens, a cyano group, and C₃-C₁₀ cycloalkyl groups optionally substituted with one or more groups chosen from C₁-C₆ alkyl groups, C₁-C₆ haloalkyl groups, and halogens;
q is 1, 2, 3 or 4;
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4, or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),

or R⁸ and R⁹ on the same carbon together form an oxo; and
each R^{b} is independently chosen from hydrogen, phenyl, and C₁-C₆ alkyl groups, wherein the C₁-C₆ alkyl groups are optionally and independently substituted with one or more groups chosen from hydroxyl, —C(O)N(R^{x})(R^{y}), cyano, 4- to 6-membered heterocyclyl, 5-membered heteroaryl optionally substituted with C₁-C₆ alkyl;
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl);

provided that at least one of R⁸ and R⁹ is independently selected from C₃-C₆ haloalkyl groups, C₃-C₆ alkyl groups, C₂-C₆ alkenyl, C₂-C₆ alkynyl, C₃-C₆ alkoxyl groups, C₃-C₆ haloalkoxyl groups, phenyl, 5- to 6-membered heteroaryl groups, and 5- to 6-membered heterocyclyl groups, or at least one R³ is a C₂-C₆ alkyl substituted by 0, 1, 2, 3, 4, 5, or 6 3- to 8-membered cycloalkyl rings or 5- or 6-membered aryl groups, or Ci alkyl substituted by 1, 2, 3, 4, 5, or 6 3- to 8-membered cycloalkyl rings or 5- or 6-membered aryl groups, or two R³ are joined to form a C₃-C₆ cycloaklyl ring.

48. The compound, tautomer, deuterated derivative, or pharmaceutically acceptable salt of embodiment 40 or 41, wherein X is O.
49. The compound, tautomer, deuterated derivative, or pharmaceutically acceptable salt of any one of embodiments 40 to 43 or 48, wherein Ring A is selected from phenyl, pyridine, pyrizine, and pyrazole.
50. The compound, tautomer, deuterated derivative, or pharmaceutically acceptable salt of any one of embodiments 40 to 43 or 48, wherein:
Ring A is a phenyl; and
one R¹ and one R^{b}, together with the atoms to which they are attached, form a pyrrole or a pyridine.
   51. The compound, tautomer, deuterated derivative, or pharmaceutically acceptable salt of any one of embodiments 40 or 48 to 50, wherein Ring B is a pyridinyl ring.
   52. The compound, tautomer, deuterated derivative, or pharmaceutically acceptable salt of any one of embodiments 40 to 42, 44, 46, or 48 to 51, wherein Ring D is selected from pyridinyl, pyrrolyl, cyclohexyl, cyclohexenyl, imidazolidinonyl, cyclobutyl, and phenyl, each of which is substituted with q instances of R⁴.
   53. The compound, tautomer, deuterated derivative, or pharmaceutically acceptable salt of any one of embodiments 40 to 42, 44, 46, or 48 to 51, wherein Ring D is selected from

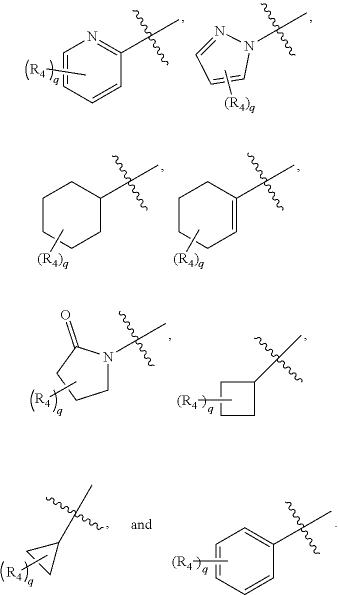

54. The compound, salt, or deuterated derivative of embodiment 53, wherein Ring D is selected from

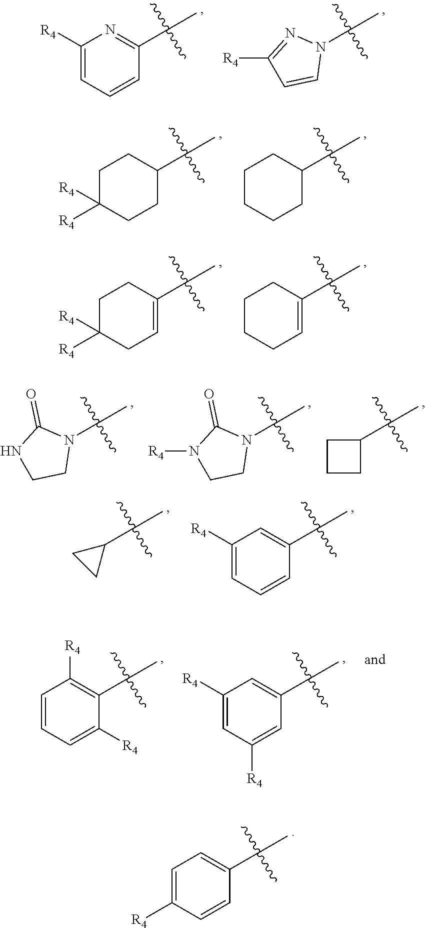

55. The compound, tautomer, deuterated derivative, or pharmaceutically acceptable salt of any one of embodiments 40 to 47, wherein R⁴ is selected from halogens, an oxo group, and —(Y)ₖ—R⁷ groups.
56. The compound, tautomer, deuterated derivative, or pharmaceutically acceptable salt of any one of embodiment 40 to 54, wherein R⁴ is selected from halogens and —(Y)ₖ—R⁷ groups.
57. The compound, tautomer, deuterated derivative, or pharmaceutically acceptable salt of any one of embodiments 40 to 56, wherein R⁴ is selected from F, Cl, CH₃,

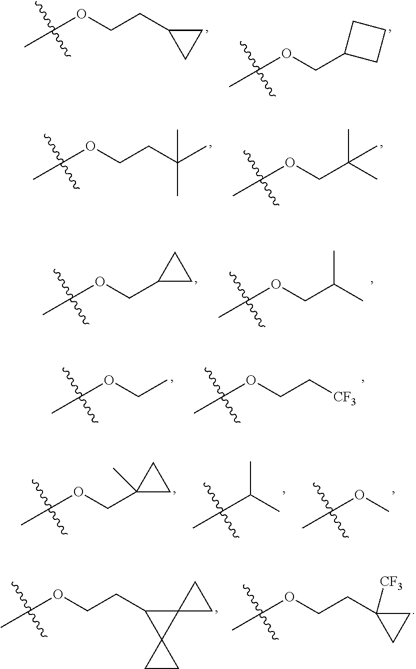

58. The compound, tautomer, deuterated derivative, or pharmaceutically acceptable salt of any one of embodiments 40 to 57, wherein m is 0.
59. The compound, tautomer, deuterated derivative, or pharmaceutically acceptable salt of any one of embodiments 40 to 57, wherein R¹ is hydroxyl.
60. The compound tautomer, deuterated derivative, or pharmaceutically acceptable salt of any one of embodiments 40 to 59, wherein R³ is a C₁-C₆ alkyl substituted with a phenyl.
61. The compound, tautomer, deuterated derivative, or pharmaceutically acceptable salt of embodiment 60, wherein R³ is benzyl.
62. The compound, tautomer, deuterated derivative, or pharmaceutically acceptable salt of any one of embodiments 40 to 59, wherein each R³ is a Ci alkyl.
63. The compound, tautomer, deuterated derivative, or pharmaceutically acceptable salt of any one of embodiments 40 to 62, wherein n is 0.
64. The compound, tautomer, deuterated derivative, or pharmaceutically acceptable salt of any one of embodiments 40 to 45 or 48 to 63, wherein Z is chosen from

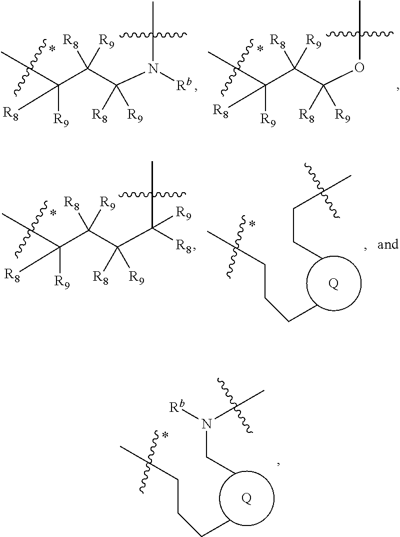

wherein [symbol] denotes the point of attachment of Z to Ring C and [symbol] denotes the point of attachment of Z to Ring A.
65. The compound, tautomer, deuterated derivative, or pharmaceutically acceptable salt of any one of embodiments 40 to 45 or 48 to 63, wherein Z is chosen from

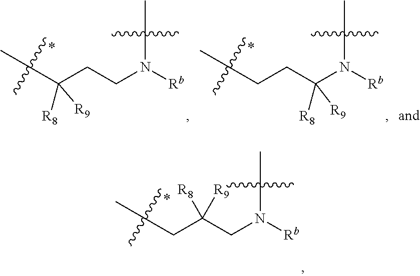

wherein [symbol] denotes the point of attachment of Z to Ring C and [symbol] denotes the point of attachment of Z to Ring A.
66. The compound, tautomer, deuterated derivative, or pharmaceutically acceptable salt of any one of embodiments 40 to 45 or 48 to 63, wherein R^{b} is selected from H, —CH₃, phenyl,

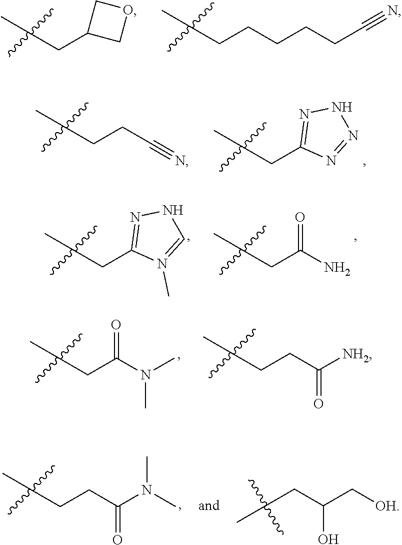

67. The compound, tautomer, deuterated derivative, or pharmaceutically acceptable salt of any one of embodiments 40 to 66, wherein each C(R⁸)(R⁹) group is independently chosen from —CH₂—, —CO—,

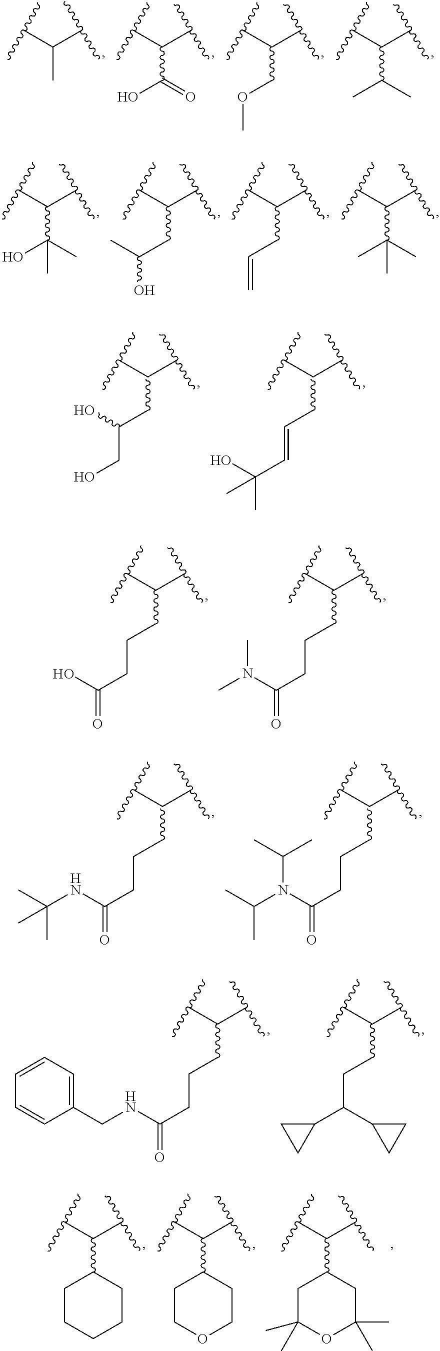

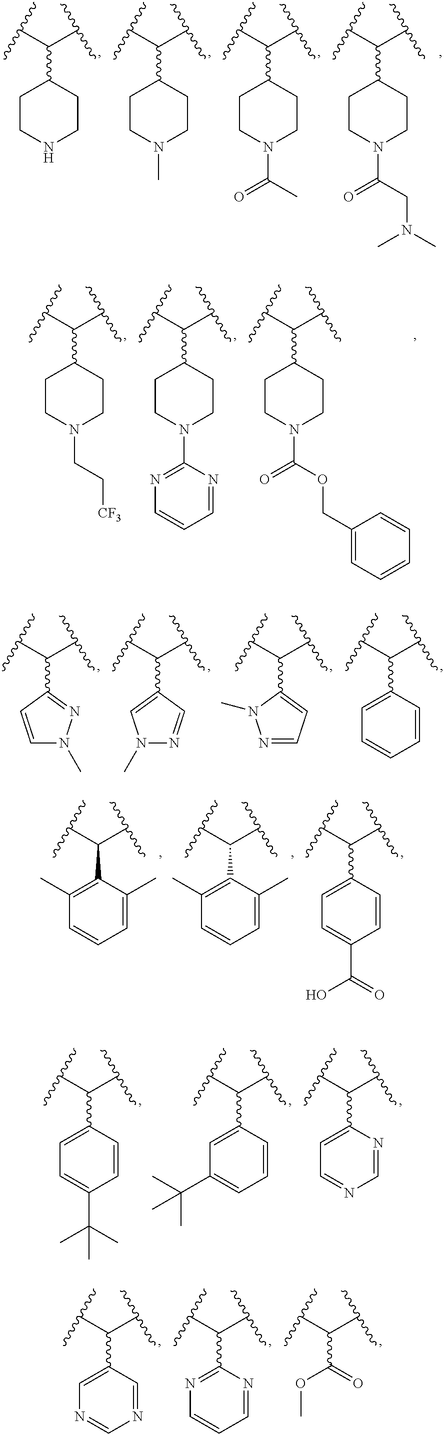

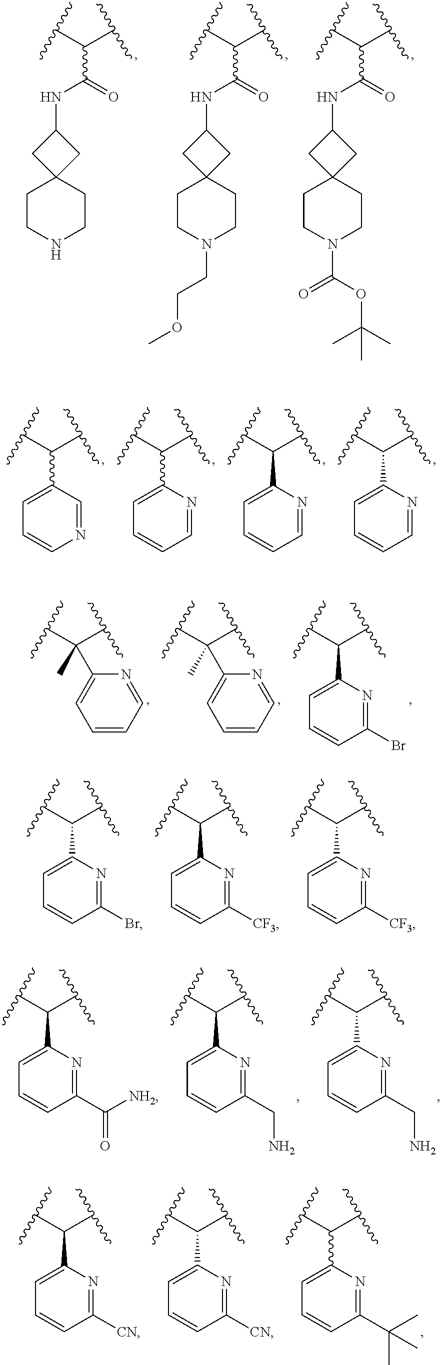

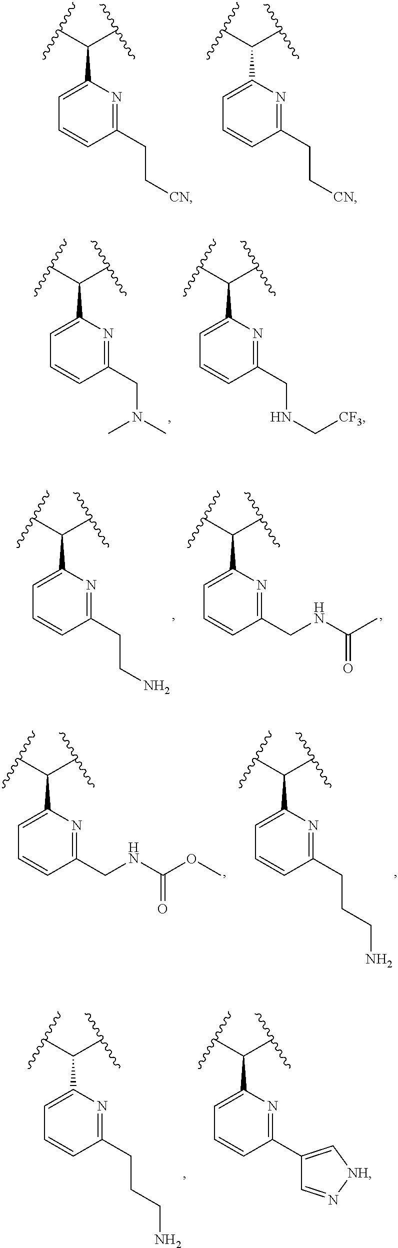

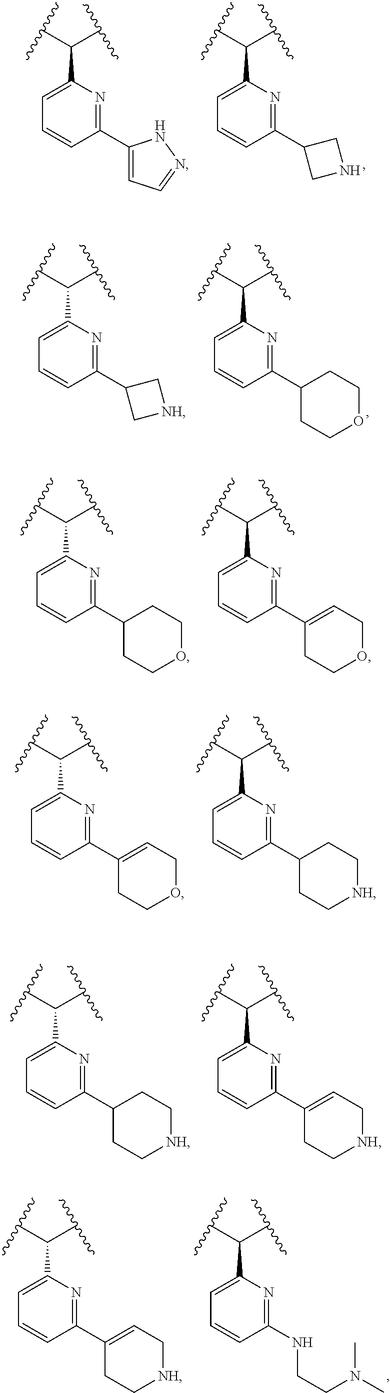

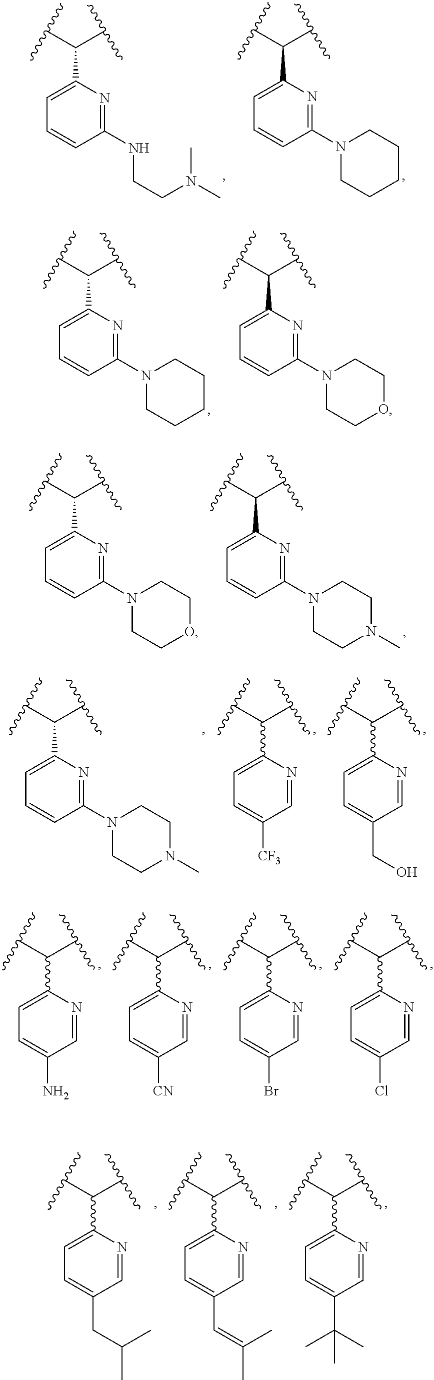

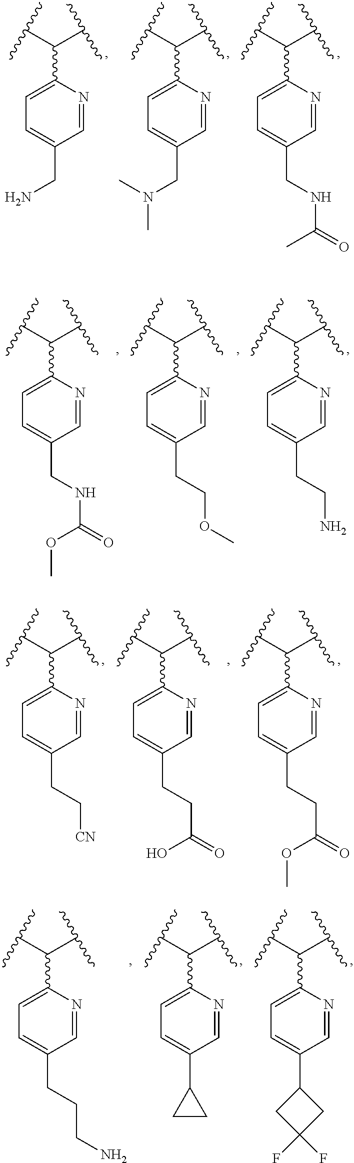

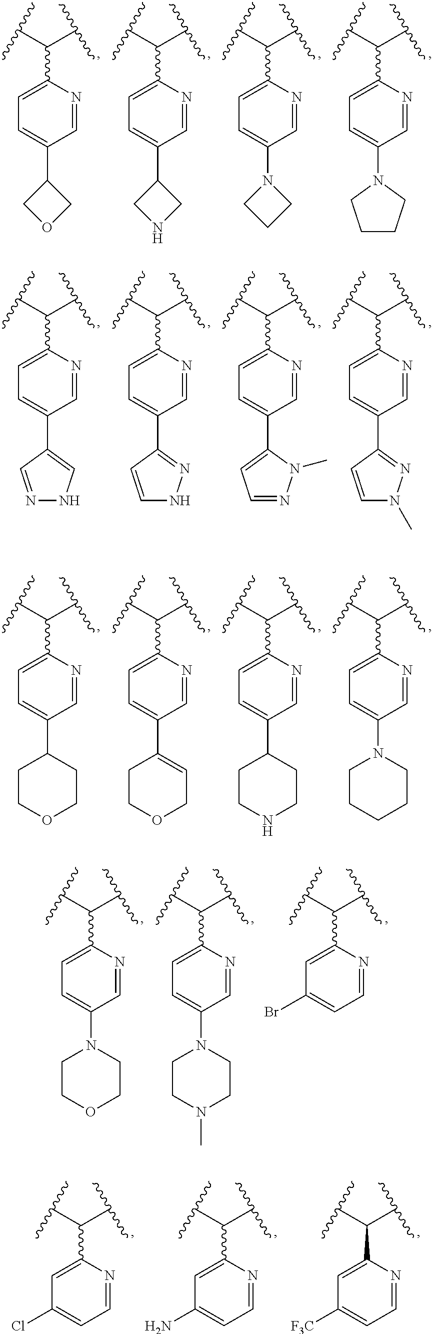

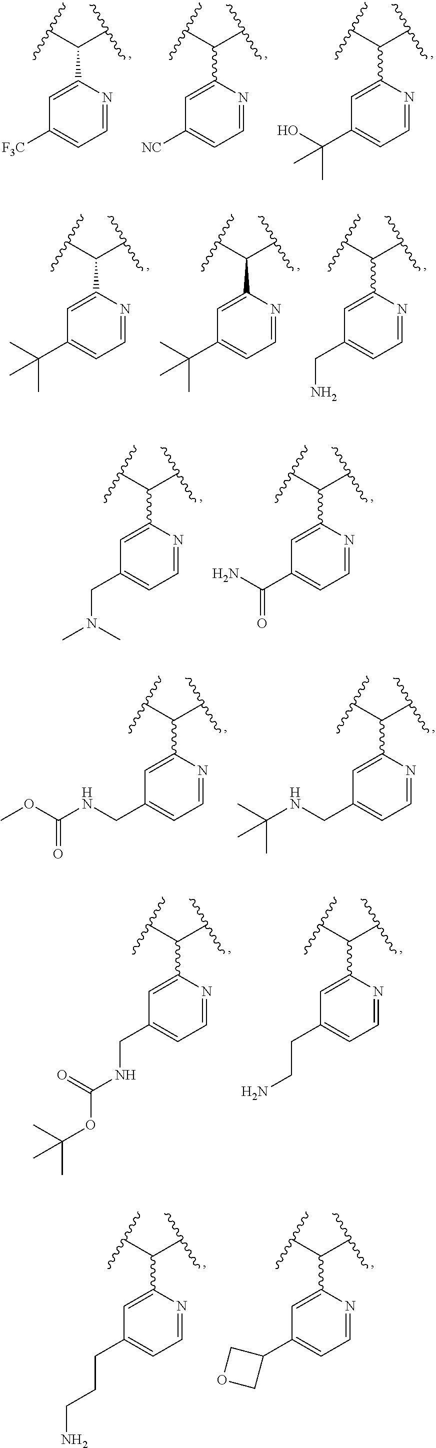

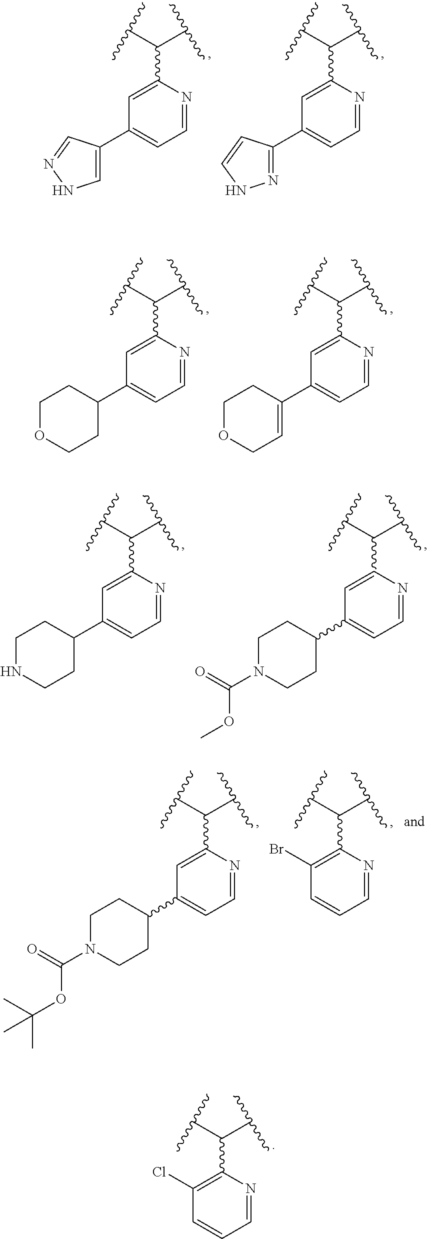

68. The compound, tautomer, deuterated derivative, or pharmaceutically acceptable salt of any one of embodiments 40 to 67, wherein each C(R⁸)(R⁹) group is independently chosen from —CH₂—,

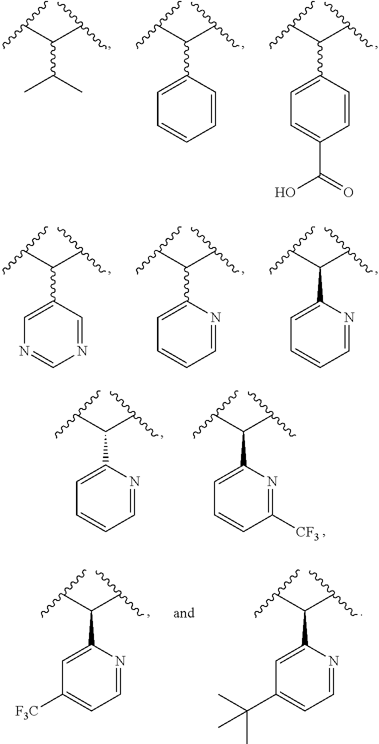

69. The compound, tautomer, deuterated derivative, or pharmaceutically acceptable salt of any one of embodiments 40 to 68, wherein

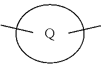

is chosen from

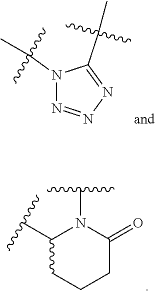 [SMILES: *c1nnnn1* ; *C1CCCC(=O)N1*]

70. A compound selected from Compounds 299-397 (Table 3B), tautomers thereof, deuterated derivative of the compound and tautomers, and pharmaceutically acceptable salts of any of the foregoing.
71. A pharmaceutical composition comprising a compound, tautomer, deuterated derivative, or pharmaceutically acceptable salt of any one of embodiments 40 to 70 and a pharmaceutically acceptable carrier.
72. The pharmaceutical composition of embodiment 71, further comprising one or more additional therapeutic agent(s).
73. The pharmaceutical composition of embodiment 72, wherein the one or more additional therapeutic agent(s) comprise(s) a compound selected from tezacaftor, ivacaftor, D-ivacaftor, and pharmaceutically acceptable salts thereof.
74. The pharmaceutical composition of embodiment 72, wherein the composition comprises tezacaftor and ivacaftor.
75. The pharmaceutical composition of embodiment 72, wherein the composition comprises tezacaftor and D-ivacaftor.
76. A pharmaceutical composition comprising:

(a) at least one compound, tautomer, deuterated derivative, or pharmaceutically acceptable salt according to of any one of embodiments 40 to 70;

(b) at least one pharmaceutically acceptable carrier; and optionally one or more of:

(c) (i) a compound chosen from tezacaftor:

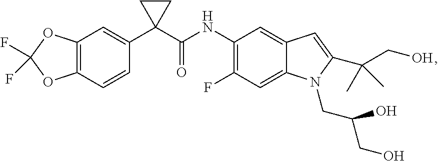 [SMILES: CC(C)(CO)c1cc2cc(NC(=O)C3(c4ccc5c(c4)OC(F)(F)O5)CC3)c(F)cc2n1C[C@@H](O)CO]

and pharmaceutically acceptable salts and deuterated derivatives thereof; and
(ii) a compound chosen from ivacaftor

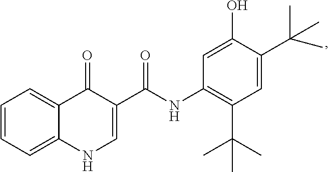 [SMILES: CC(C)(C)c1cc(C(C)(C)C)c(NC(=O)c2c[nH]c3ccccc3c2=O)cc1O]

### D-ivacaftor:

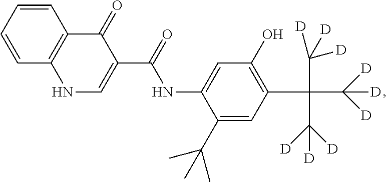 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

and pharmaceutically acceptable salts and deuterated derivatives thereof.
77. A method of treating cystic fibrosis comprising administering to a patient in need thereof a compound, tautomer, deuterated derivative, or pharmaceutically acceptable salt of any one of embodiments 40 to 70 or a pharmaceutical composition according to any one of embodiments 71 to 76.
78. The method of embodiment 77, further comprising administering to the patient one or more additional therapeutic agent(s) prior to, concurrent with, or subsequent to the compound or the pharmaceutical composition.
79. The method of embodiment 78, wherein the one or more additional therapeutic agent(s) comprise(s) a compound selected from tezacaftor, ivacaftor, D-ivacaftor, lumacaftor, and pharmaceutically acceptable salts thereof.
80. The method of embodiment 79, wherein the one or more additional therapeutic agent(s) comprise(s) tezacaftor and ivacaftor.
81. The method of embodiment 79, wherein the one or more additional therapeutic agent(s) comprise(s) ivacaftor and D-ivacaftor.
82. The compound, tautomer, deuterated derivative, or pharmaceutically acceptable salt of any one of embodiments 40 to 70 or the pharmaceutical composition according to any one of embodiments 71 to 76 for use in the treatment of cystic fibrosis.
83. The compound, tautomer, deuterated derivative, or pharmaceutically acceptable salt of any one of embodiments 40 to 70 or the pharmaceutical composition according to any one of embodiments 71 to 76 for use in the manufacture of a medicament for the treatment of cystic fibrosis.
84. A compound selected from Compounds 398-436 (Table 4), tautomers thereof, deuterated derivatives of the compounds and tautomers and pharmaceutically acceptable salts of any of the foregoing.
85. A pharmaceutical composition comprising a compound, tautomer, deuterated derivative, or pharmaceutically acceptable salt of embodiments 84, and a pharmaceutically acceptable carrier.
86. The pharmaceutical composition of embodiment 85, further comprising one or more additional therapeutic agents.
87. The pharmaceutical composition of embodiment 86, wherein the one or more additional therapeutic agents are selected from tezacaftor, ivacaftor, D-ivacaftor, lumacaftor, and pharmaceutically acceptable salts thereof.
88. The pharmaceutical composition of embodiment 87, wherein the composition comprises tezacaftor and ivacaftor.
89. The pharmaceutical composition of embodiment 87 wherein the composition comprises tezacaftor and D-ivacaftor.
90. A pharmaceutical composition comprising:

(a) at least one compound, tautomer, deuterated derivative, or pharmaceutically acceptable salt according to embodiment 84;

(b) at least one pharmaceutically acceptable carrier; and optionally one or more of:

(c) (i) a compound chosen from tezacaftor:

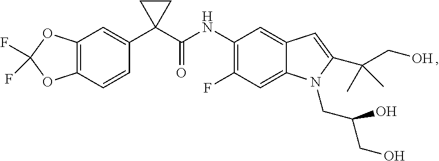 [SMILES: CC(C)(CO)c1cc2cc(NC(=O)C3(c4ccc5c(c4)OC(F)(F)O5)CC3)c(F)cc2n1C[C@@H](O)CO]

and pharmaceutically acceptable salts and deuterated derivatives thereof; and
(ii) a compound chosen from ivacaftor

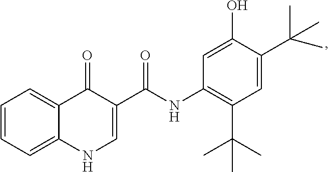 [SMILES: CC(C)(C)c1cc(C(C)(C)C)c(NC(=O)c2c[nH]c3ccccc3c2=O)cc1O]

### D-ivacaftor

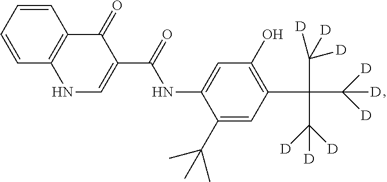 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

and pharmaceutically acceptable salts and deuterated derivatives thereof.
91. A method of treating cystic fibrosis comprising administering to a patient in need thereof a compound, tautomer, deuterated derivative, or pharmaceutically acceptable salt of embodiment 84 or a pharmaceutical composition according to embodiment 85.
92. The method of embodiment 91, further comprising administering to the patient one or more additional therapeutic agent(s) prior to, concurrent with, or subsequent to the compound or the pharmaceutical composition.
93. The method of embodiment 91, wherein the one or more additional therapeutic agent(s) comprise(s) a compound selected from tezacaftor, ivacaftor, D-ivacaftor, lumacaftor, and pharmaceutically acceptable salts thereof.
94. The method of embodiment 93, wherein the one or more additional therapeutic agent(s) comprise(s) tezacaftor and ivacaftor.
95. The method of embodiment 93, wherein the one or more additional therapeutic agent(s) comprise(s) tezacaftor and D-ivacaftor.
96. The compound, tautomer, deuterated derivative, or pharmaceutically acceptable salt of embodiment 84 or the pharmaceutical composition according to embodiment 85 for use in the treatment of cystic fibrosis.
97. The compound, tautomer, deuterated derivative, or pharmaceutically acceptable salt of embodiment 84 or the pharmaceutical composition according to embodiment 85 for use in the manufacture of a medicament for the treatment of cystic fibrosis.

### EXAMPLES

### Abbreviation List

### ACN: Acetonitrile

Boc anhydride ((Boc)₂O): Di-tert-butyl dicarbonate
CDI: Carbonyl diimidazole
COMU: (1-Cyano-2-ethoxy-2-oxoethylidenaminooxy)dimethylamino-morpholino-carbenium hexafluorophosphate
DABCO: 1,4-Diazabicyclo[2.2.2]octane
DBU: 1,8-Diazabicyclo(5.4.0)undec-7-ene

### DCM: Dichloromethane

### DI: Deionized

DIAD: Diisopropyl azodicarboxylate

### DIEA: (DIPEA; N,N-diisopropylethylamine)

### DMA: N,N-Dimethylacetamide

### DMAP: 4-Dimethylaminopyridine

### DMF: N,N-Dimethylformamide

DMSO: Dimethyl sulfoxide
EA: Ethyl acetate
EDC: 1-Ethyl-3-(3-dimethylaminopropyl)carbodiimide
ELSD: Evaporative light scattering detector
Et₂O: Diethyl ether
EtOAc: Ethyl acetate

### EtOH: Ethanol

HATU: 1-[Bis(dimethylamino)methylene]-1H-1,2,3-triazolo[4,5-b]pyridinium 3-oxid hexafluorophosphate
HPLC: High performance liquid chromatography

### HMPA: Hexamethylphosphoramide

Hoveyda-Grubbs 2^{nd} Generation catalyst: (1,3-Bis-(2,4,6-trimethylphenyl)-2-imidazolidinylidene)dichloro(o-isopropoxyphenylmethylene)ruthenium, Dichloro[1,3-bis(2,4,6-trimethylphenyl)-2-imidazolidinylidene](2-isopropoxyphenylmethylene)ruthenium(II)

### IPA: Isopropanol

[Ir{dF(CF3)ppy}2(dtbpy)]PF₆: [4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate
LAH: Lithium aluminium hydride
LC: Liquid chromatography
LDA: Lithium diisopropylamide

### MeCN: Acetonitrile

### MeOH: Methanol

MTBE: Methyl tert-butyl ether

### MeTHF or 2-MeTHF: 2-Methyltetrahydrofuran

NMP: N-Methyl-2-pyrrolidone

### NMM: N-Methylmorpholine

### Pd(dppf)Cl₂: [1,1′-Bis(diphenylphosphino)ferrocene]dichloropalladium(II)

### PTFE: Polytetrafluoroethylene

rt: Room temperature
SFC: Supercritical fluid chromatography
TBS-Cl: tert-Butyldimethylsilyl chloride

### TEA: Triethylamine

TFA: Trifluoroacetic acid

### THF: Tetrahydrofuran

### TMS: Trimethylsilyl

TMSCl: Trimethylsilyl chloride
TPPO-DIAD complex: a complex of triphenylphosphine oxide with diisopropyl azodicarboxylate
p-TsOH: p-Toluenesulfonic Acid

### UPLC: Ultra Performance Liquid Chromatography

### General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. ¹⁹F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before fourrier transform. ¹⁹F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Final purity of compounds was determined by reversed phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range. Optical purity of methyl (2S)-2,4-dimethyl-4-nitro-pentanoate was determined using chiral gas chromatography (GC) analysis on an Agilent 7890A/MSD 5975C instrument, using a Restek Rt-βDEXcst (30 m×0.25 mm×0.25 μm_df) column, with a 2.0 mL/min flow rate (H₂ carrier gas), at an injection temperature of 220° C. and an oven temperature of 120° C., 15 minutes.

### General UPLC/HPLC Analytical Methods

LC Method A:

Analytical reverse phase UPLC using an Acquity UPLC BEH C₁₈ column (50×2.1 mm, 1.7 μm particle) made by Waters (pn: 186002350), and a dual gradient run from 1-99% mobile phase B over 2.9 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC Method B:

Merckmillipore Chromolith SpeedROD C₁₈ column (50×4.6 mm) and a dual gradient run from 5-100% mobile phase B over 6 minutes. Mobile phase A=water (0.1% CF₃CO₂H). Mobile phase B=acetonitrile (0.1% CF₃CO₂H).

LC Method C:

Merckmillipore Chromolith SpeedROD C₁₈ column (50×4.6 mm) and a dual gradient run from 5-100% mobile phase B over 12 minutes. Mobile phase A=water (0.1% CF₃CO₂H). Mobile phase B=acetonitrile (0.1% CF₃CO₂H).

LC Method D:

Acquity UPLC BEH C₁₈ column (30×2.1 mm, 1.7 μm particle) made by Waters (pn: 186002349), and a dual gradient run from 1-99% mobile phase B over 1.0 minute. Mobile phase A=H₂O (0.05% CF₃CO₂H). Mobile phase B=CH₃CN (0.035% CF₃CO₂H). Flow rate=1.5 mL/min, injection volume=1.5 μL, and column temperature=60° C.

LC Method E:

LuNa column C₁₈ (2) 50×3 mm, 3 μm. run: 2.5 min. Mobile phase: Initial 95% H₂O containing 0.1% formic acid/5% MeCN containing 0.1% formic acid, linear gradient to 95% MeCN containing 0.1% formic acid over 1.3 min, hold 1.2 min at 95% MeCN containing 0.1% formic acid. Temperature: 45° C., Flow: 1.5 mL/min.

LC Method F:

SunFire column C₁₈ 75×4.6 mm 3.5 μm, run: 6 min. Mobile phase conditions: Initial 95% H₂O+0.1% formic acid/5% MeCN+0.1% formic acid, linear gradient to 95% MeCN for 4 min, hold for 2 min at 95% MeCN. T: 45° C., Flow: 1.5 mL/min.

LC Method G:

Analytical reverse phase UPLC using an Acquity UPLC BEH C₁₈ column (50×2.1 mm, 1.7 μm particle) made by Waters (pn: 186002350), and a dual gradient run from 30-99% mobile phase B over 2.9 minutes. Mobile phase A=H₂O (0.05% CF₃CO₂H). Mobile phase B=MeCN (0.035% CF₃CO₂H). Flow rate=1.2 mL/min, injection volume=1.5 μL, and column temperature=60° C.

LC Method H:

Water Cortex 2.7μ C₁₈ (3.0 mm×50 mm) column, Temp: 55° C.; Flow: 1.2 mL/min; Mobile phase: 100% water with 0.1% trifluoroacetic (TFA) acid then 100% acetonitrile with 0.1% TFA acid, gradient 5% to 100% B over 4 min, with stay at 100% B for 0.5 min, equilibration to 5% B over 1.5 min.

LC Method I:

Reverse phase UPLC using an Acquity UPLC BEH C₁₈ column (30×2.1 mm, 1.7 μm particle) made by Waters (pn: 186002349), and a dual gradient run from 30-99% mobile phase B over 1.0 minutes. Mobile phase A=H₂O (0.05% CF₃CO₂H). Mobile phase B=CH₃CN (0.035% CF₃CO₂H). Flow rate=1.5 mL/min, injection volume=1.5 μL, and column temperature=60° C.

LC Method J:

Analytical reverse phase UPLC using an Acquity UPLC BEH C₁₈ column (30×2.1 mm, 1.7 μm particle) made by Waters (pn: 186002349), and a dual gradient run from 1-99% mobile phase B over 1.2 minutes. Mobile phase A=water (0.05% trifluoroacetic acid). Mobile phase B=acetonitrile (0.035% trifluoroacetic acid). Flow rate=1.5 mL/min, injection volume=1.5 μL, and column temperature=60° C.

LC Method K:

Analytical reverse phase UPLC using an Acquity UPLC BEH C₁₈ column (50×2.1 mm, 1.7 μm particle) made by Waters (pn: 186002350), and a dual gradient run from 1-99% mobile phase B over 5.0 minutes. Mobile phase A=water (0.05% trifluoroacetic acid). Mobile phase B=acetonitrile (0.035% trifluoroacetic acid). Flow rate=1.2 mL/min, injection volume=1.5 μL, and column temperature=60° C.

LC Method L:

Analytical reverse phase UPLC using an Acquity UPLC BEH C₁₈ column (50×2.1 mm, 1.7 μm particle) made by Waters (pn: 186002350), and a dual gradient run from 1-99% mobile phase B over 2.5 minutes. Mobile phase A=water (0.05% trifluoroacetic acid). Mobile phase B=acetonitrile (0.035% trifluoroacetic acid). Flow rate=1.2 mL/min, injection volume=1.5 μL, and column temperature=60° C.

LC Method M:

Reverse phase UPLC using an Acquity UPLC BEH C₁₈ column (50×2.1 mm, 1.7 μm particle) made by Waters (pn: 186002350), and a dual gradient run from 50-99% mobile phase B over 2.9 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC Method N:

Zorbax C₁₈ 4.6×50 mm 3.5 μm. Flow: 2.0 mL/min, 95% water (0.1% trifluoroacetic acid)+5% acetonitrile (0.1% trifluoroacetic acid) to 95% acetonitrile (0.1% trifluoroacetic acid) gradient (2.0 min) then hold at 95% acetonitrile (0.1% trifluoroacetic acid) for 1.0 min.

LC Method O:

Kinetex C₁₈ 4.6×50 mm 2.6 μm. Temp: 45° C., Flow: 2.0 mL/min, Run Time: 3 min. Mobile phase: Initial 95% water (0.1% formic acid) and 5% acetonitrile (0.1% formic acid) linear gradient to 95% acetonitrile (0.1% formic acid) for 2.0 min then hold at 95% acetonitrile (0.1% formic acid) for 1.0 min.

LC Method P:

Reverse phase HPLC using a Kinetex C₁₈ column (50×3.0 mm) and a dual gradient run from 5-100% mobile phase B over 6 minutes. Mobile phase A=H₂O (0.1% CF₃CO₂H). Mobile phase B=CH₃CN (0.1% CF₃CO₂H). Flow rate=1.5 mL/min, injection volume=2 μL, and column temperature=60° C.

LC Method Q:

Reverse phase HPLC-MS using an Onyx Monolithic C₁₈ column (50×4.6 mm) sold by Phenomenex (pn: CHO-7644), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=12 mL/min, injection volume=50 μL, and column temperature=25° C.

LC Method R:

Reverse phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC Method S:

Reverse phase HPLC-MS using an Onyx Monolithic C₁₈ column (50×4.6 mm) sold by Phenomenex (pn: CHO-7644), and a dual gradient run from 1-99% mobile phase B over 2.9 minutes. Mobile phase A=H₂O (0.05% CF₃CO₂H). Mobile phase B=CH₃CN (0.035% CF₃CO₂H). Flow rate=12 mL/min, injection volume=50 μL, and column temperature=25° C.

LC Method T:

HPLC-MS using an Onyx Monolithic C₁₈ column (50×4.6 mm) sold by Phenomenex (pn: CHO-7644), and a dual gradient run from 1-99% mobile phase B over 1.2 minutes. Mobile phase A=H₂O (0.05% CF₃CO₂H). Mobile phase B=CH₃CN (0.035% CF₃CO₂H). Flow rate=12 mL/min, injection volume=50 μL, and column temperature=25° C.

LC Method U:

UPLC using an Acquity UPLC BEH C₁₈ column (30×2.1 mm, 1.7 μm particle) made by Waters (pn: 186002349), and a dual gradient run from 50-99% mobile phase B over 1.0 minutes. Mobile phase A=H₂0 (0.05% CF₃CO₂H). Mobile phase B=CH₃CN (0.035% CF₃CO₂H). Flow rate=1.5 mL/min, injection volume=1.5 μL, and column temperature=60° C.

LC Method V:

Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 2.9 minutes. Mobile phase A=H₂0 (0.05% NH₄HCO₂). Mobile phase B=CH₃CN. Flow rate=1.2 mL/min, injection volume=1.5 μL, and column temperature=60° C.

### Example 1: Preparation of tert-Butyl 2,6-dichloropyridine-3-carboxylate

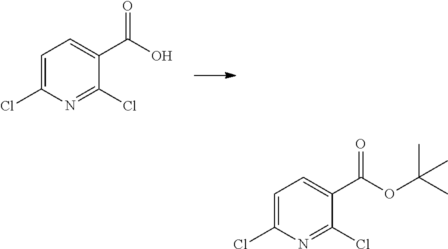

A solution of 2,6-dichloropyridine-3-carboxylic acid (10 g, 52.08 mmol) in tetrahydrofuran (210 mL) was treated successively with di-tert-butyl dicarbonate (17 g, 77.89 mmol) and 4-(dimethylamino)pyridine (3.2 g, 26.19 mmol) and stirred overnight at room temperature. At this point, hydrochloric acid 1N (400 mL) was added, and the mixture was stirred vigorously for about 10 min. The product was extracted with ethyl acetate (2×300 mL), and the combined organic layers were washed with water (300 mL) and brine (150 mL) and dried over sodium sulfate, filtered and concentrated under reduced pressure to give 12.94 g (96% yield) of tert-butyl 2,6-dichloropyridine-3-carboxylate as a colorless oil. ¹H NMR (300 MHz, CDCl₃) δ 1.60 (s, 9H), 7.30 (d, J=7.9 Hz, 1H), 8.05 (d, J=8.2 Hz, 1H). ESI-MS m/z calc. 247.02, found 248.1 (M+1)⁺; Retention time: 1.79 min (LC Method A).

### Example 2: Preparation of 6-Fluoropyridine-2-sulfonamide

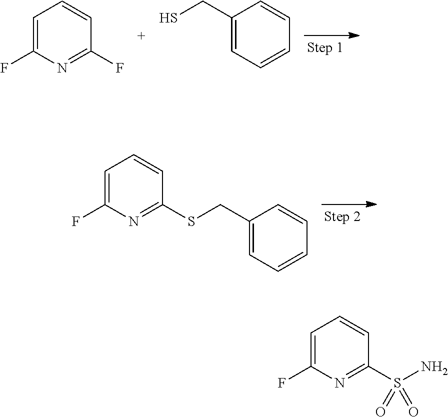 [SMILES: Fc1cccc(F)n1 ; SCc1ccccc1 ; Fc1cccc(SCc2ccccc2)n1 ; NS(=O)(=O)c1cccc(F)n1]

### Step 1: 2-Benzylsulfanyl-6-fluoro-pyridine

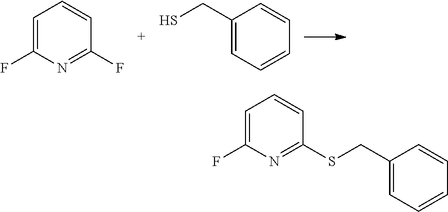

2,6-Difluoropyridine (200 g, 1.738 mol) was dissolved in dimethyl sulfoxide (2 L) in a 5 L three-necked round-bottomed flask equipped with an overhead stirrer, temperature probe and addition funnel. Cesium carbonate (572.4 g, 1.757 mol) was added. Phenylmethanethiol (206 mL, 1.755 mol) was added dropwise via addition funnel. An exotherm was observed during the addition. The temperature rose to approximately 40° C. The reaction was stirred overnight at room temperature. The reaction was poured into water and extracted with dichloromethane. The extract was washed twice with water and filtered over a small plug of silica gel. The plug was eluted with dichloromethane and the filtrate was evaporated in vacuo to afford 2-benzylsulfanyl-6-fluoro-pyridine (366 g, 96%) as a peach-colored oil that solidified under vacuum to huge blocky plates. ¹H NMR (400 MHz, Chloroform-d) δ 7.58 (q, J=7.9 Hz, 1H), 7.48-7.41 (m, 2H), 7.36-7.25 (m, 3H), 7.06 (dd, J=7.6, 2.1 Hz, 1H), 6.62 (dd, J=7.9, 2.6 Hz, 1H), 4.43 (s, 2H).

### Step 2: 6-Fluoropyridine-2-sulfonamide

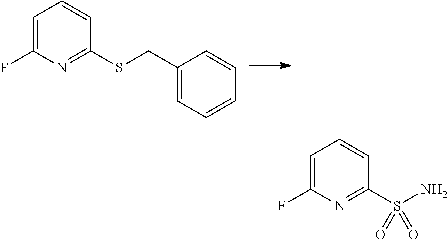 [SMILES: Fc1cccc(SCc2ccccc2)n1 ; NS(=O)(=O)c1cccc(F)n1]

2-Benzylsulfanyl-6-fluoro-pyridine (303.2 g, 1.383 mol) was dissolved in chloroform (2.0 L) in a 12 L three-necked round-bottomed flask equipped with an overhead stirrer and temperature probe. Water (1.5 L) was added and the mixture was cooled in an ice bath to 0° C. and vigorously stirred. Chlorine gas from a lecture bottled was bubbled vigorously into the reaction by way of a Pasteur pipet inserted through a septum on the third neck of the flask. A white precipitate rapidly formed. An exotherm was observed during the addition. The chlorine addition was stopped when the temperature rose to 20° C. The reaction was allowed to cool again before the addition of more chlorine gas. Dosing was continued until the reaction turned a yellowish-green color and stayed that way after stirring for 30 min. At this point, no further exotherms were observed. The reaction was poured into a solution of 40% aqueous sodium bisulfite. The organic layer was separated and the aqueous was extracted with another portion of chloroform. The organic layers were combined, dried over magnesium sulfate, filtered, and evaporated in vacuo to afford a slightly yellow oil. The oil was dissolved in dichloromethane (1.5 L) and added dropwise to ammonium hydroxide (1.5 L of 40% w/v, 17.12 mol) in a 12 L three-necked round-bottomed flask equipped with an overhead stirrer, temperature probe, and addition funnel. The ammonium hydroxide solution was cooled to 0° C. in an ice-bath before the addition. The addition rate was adjusted so the temperature of the reaction stayed below 10° C. The resulting greenish-yellow solution was stirred for an hour and poured into ice. The layers were separated (the organic layer was dark green) and the aqueous layer was extracted with more dichloromethane. The organic layers were discarded. The aqueous layer was cooled in an ice bath and concentrated aqueous hydrochloric acid was added in portions to the aqueous layer until the pH was strongly acidic. The resulting mixture was stirred as each portion was added. The resulting aqueous solution was extracted twice with ethyl acetate. The organic layers were combined, dried over magnesium sulfate, filtered, and evaporated in vacuo to afford a light brown solid. The solid was mixed with dichloromethane (approximately 500 mL) and stirred with a magnetic stirbar until most of the large clumps had broken up. Approximately 1.5 L of pentane was added which precipitated a lot of light brown solid. The resulting mixture was stirred briefly and then filtered. The filter cake was washed with pentane and dried in vacuo to afford 6-fluoropyridine-2-sulfonamide (204.1 g, 84%) as a light brown solid. ¹H NMR (300 MHz, dimethyl sulfoxide-d₆) δ 8.52-8.11 (m, 1H), 7.89 (dd, J=7.8, 2.7 Hz, 1H), 7.67 (s, 2H), 7.57-7.44 (m, 1H).

### Example 3: Preparation of tert-butyl (4S)-4-(3-hydroxypropyl)-2,2-dimethyl-pyrrolidine-1-carboxylate

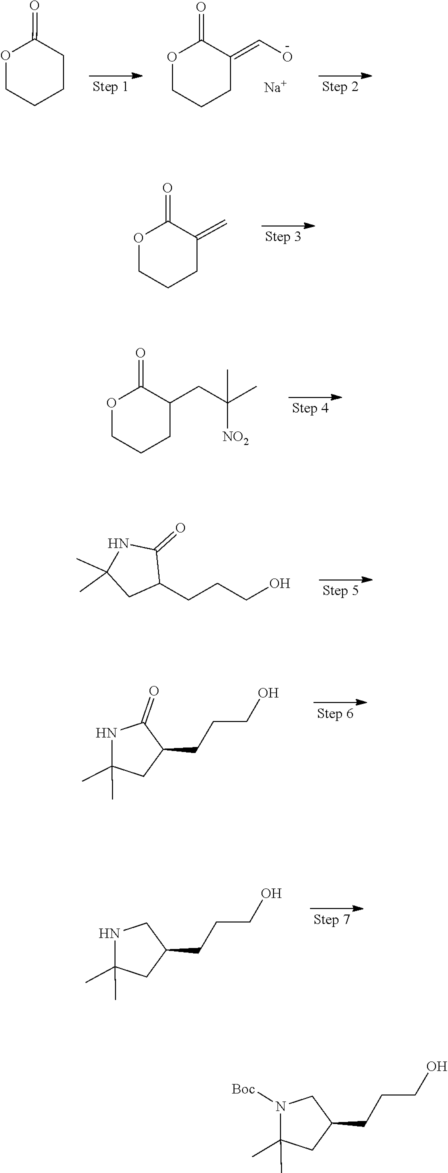

### Step 1: (E)-(2-Oxotetrahydropyran-3-ylidene)methanolate (Sodium Salt)

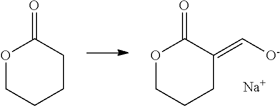 [SMILES: O=C1CCCCO1 ; O=C1OCCC/C1=C\[O-] ; [Na+]]

A 5 L, 3-neck round bottom flask was fitted with a mechanical stirrer, a heating mantle, an addition funnel, a J-Kem temperature probe/controller and a nitrogen inlet/outlet. The vessel was charged under a nitrogen atmosphere with sodium hydride (59.91 g of 60% w/w, 1.498 mol) followed by heptane (1.5 L) which provided a grey suspension. Stirring was commenced and the pot temperature was recorded at 19° C. The vessel was then charged with ethyl alcohol (3.451 g, 74.91 mmol) added via syringe which resulted in gas evolution. The addition funnel was charged with a clear pale yellow solution of tetrahydropyran-2-one (150 g, 1.498 mol) and ethyl formate (111 g, 1.50 mol). The solution was added dropwise over 1 h which resulted in gas evolution and a gradual exotherm to 45° C. The resulting thick white suspension was then heated to 65° C. for 2 h and then allowed to cool to room temperature. The mixture was continued to stir at room temperature overnight (about 10 h). The reaction mixture was vacuum filtered through a glass frit Buchner funnel (medium porosity) under a stream of nitrogen. The filter cake was displacement washed with heptane (2×250 mL) and pulled for a few min. The slightly heptane wet cake was transferred to a glass tray and dried in a vacuum oven at 45° C. for 15 h to provide a white solid (205 g, 1.36 mol, 91% yield) as the desired product, (E)-(2-oxotetrahydropyran-3-ylidene)methanolate (sodium salt).

### Step 2: 3-Methylenetetrahydropyran-2-one

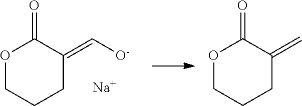

A 5 L, 3-neck round bottom flask was fitted with a mechanical stirrer, a heating mantle, an addition funnel, a J-Kem temperature probe/controller and a nitrogen inlet/outlet. The vessel was charged under a nitrogen atmosphere with (E)-(2-oxotetrahydropyran-3-ylidene)methanolate (sodium salt) (205 g, 1.366 mol) (205 g, 1.366 mol) and tetrahydrofuran (1640 mL) which provided a white suspension. Stirring was commenced and the pot temperature was recorded at 19° C. The vessel was then charged with paraformaldehyde (136.6 g, 4.549 mol) added as a solid in one portion. The resulting suspension was heated to 63° C. and the condition was maintained for 15 h. Upon heating the reaction mixture became slightly gelatinous. The white gelatinous mixture was concentrated under reduced pressure to remove most of the tetrahydrofuran. The remaining residue was partitioned with ethyl acetate (1000 mL), saturated sodium chloride (500 mL) and saturated sodium hydrogen carbonate (500 mL) in a separatory funnel. The organic was removed and the residual aqueous was extracted with ethyl acetate (5×300 mL). The combined organic was dried over sodium sulfate (500 g) and then vacuum filtered through a glass frit Buchner funnel with a 20 mm layer of celite. The filter cake was displacement washed with ethyl acetate (250 mL). The clear filtrate was concentrated under reduced pressure to provide a clear pale yellow oil (135 g) as the desired crude product. The material was purified by silica gel column flash chromatography (liquid load) eluting with a gradient of 100% hexane to 60% ethyl acetate in hexane over 1 h collecting 450 mL fractions. The product was detected by TLC analysis on silica gel eluting with 3:1 hexanes/ethyl acetate and visualized under UV. The product fractions were combined and concentrated under reduced pressure to provide a clear, colorless oil (132 g, 1.18 mol, 72% yield containing 16 wt % residual ethyl acetate by NMR) as the desired product, 3-methylenetetrahydropyran-2-one. ¹H NMR (400 MHz, dimethyl sulfoxide-d₆) δ 6.18 (q, J=1.9 Hz, 1H), 5.60 (q, J=1.9 Hz, 1H), 4.40-4.26 (m, 2H), 2.61 (ddt, J=7.0, 6.3, 2.0 Hz, 2H), 1.90-1.75 (m, 2H).

### Step 3: 3-(2-Methyl-2-nitro-propyl)tetrahydropyran-2-one

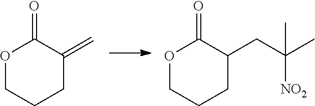

A 5000 mL, 3-neck round bottom flask was fitted with a mechanical stirrer, a cooling bath used as secondary containment, a J-Kem temperature probe, an addition funnel and a nitrogen inlet/outlet. The vessel was charged under a nitrogen atmosphere with 2-nitropropane (104.9 g, 1.177 mol). Stirring was commenced and the pot temperature was recorded at 19° C. The vessel was then charged with 1,8-diazabicyclo[5.4.0]undec-7-ene (22.41 g, 147.2 mmol) added neat in one portion which resulted in a clear light yellow solution. No exotherm was observed. The addition funnel was charged with a solution of 3-methylenetetrahydropyran-2-one (110 g, 981.0 mmol) in acetonitrile (1100 mL) which was added dropwise over 1 h which resulted in a clear light yellow solution and a gradual exotherm to 24° C. The reaction mixture was continued to stir at room temperature for 3.5 h and then concentrated under reduced pressure. The remaining residue was dissolved in dichloromethane (1000 mL) and partitioned with 500 mL of a 3:2 mixture of 1 molar citric acid solution/saturated sodium chloride solution. The resulting organic phase was a clear pale blue solution and the aqueous phase was a slightly cloudy very pale blue solution. The organic was removed and the residual aqueous was extracted with dichloromethane (300 mL). The combined organic was washed with saturated sodium chloride solution (300 mL), dried over sodium sulfate (250 g) and then filtered through a glass frit Buchner funnel. The filtrate was concentrated under reduced pressure to a volume of about 200 mL. The clear pale blue dichloromethane solution was diluted with methyl tert-butyl ether (1500 mL) and the cloudy solution was concentrated under reduced pressure to a volume of about 200 mL which provided a suspension. The mixture was again diluted with methyl tert-butyl ether (1500 mL) and concentrated under reduced pressure to a volume of about 250 mL. The resulting suspension was allowed to stand at room temperature overnight (about 12 h). The solid was collected by vacuum filtration in a glass frit Buchner funnel and the filter cake was displacement washed with cold methyl tert-butyl ether (2×150 mL) and then pulled for 30 min. The material was further dried in a vacuum oven at 45° C. for 5 h to provide (160 g, 0.795 mol, 81% yield) of a white solid as the desired product, 3-(2-methyl-2-nitro-propyl)tetrahydropyran-2-one. ¹H NMR (400 MHz, dimethyl sulfoxide-d₆) δ 4.34 (ddd, J=11.1, 9.3, 4.3 Hz, 1H), 4.20 (dt, J=11.1, 5.1 Hz, 1H), 2.75-2.62 (m, 1H), 2.56 (dd, J=14.9, 5.2 Hz, 1H), 2.01-1.89 (m, 2H), 1.89-1.67 (m, 2H), 1.55 (d, J=6.0 Hz, 6H), 1.44 (dddd, J=12.8, 11.5, 8.1, 6.6 Hz, 1H).

### Step 4: 3-(3-Hydroxypropyl)-5,5-dimethyl-pyrrolidin-2-one

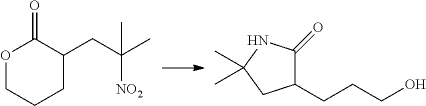

A 1000 mL, 3-neck round bottom flask was fitted with a Teflon stir bar, a heating mantle, a J-Kem temperature probe/controller and rubber septums. The vessel was charged with 3-(2-methyl-2-nitro-propyl)tetrahydropyran-2-one (25 g, 124.2 mmol) and ethyl alcohol (375 mL) which provided a white suspension. Stirring was commenced and the suspension was heated to 40° C. for 10 min which provided a clear colorless solution. The vessel was then fitted with a gas dispersion tube and the solution was degassed with nitrogen for 15 min. The vessel was then charged with Raney Nickel (8.019 g of 50% w/w, 68.31 mmol) and the vessel was then fitted with the septums. The vessel was evacuated and placed under a hydrogen atmosphere. The process was repeated for three cycles. The vessel was then placed under 1 atmosphere of hydrogen and the reaction mixture was gradually heated to 60° C. The reaction was continued to stir at 60° C. for 24 h. After cooling to room temperature, the vessel was fitted with a gas dispersion tube and the reaction mixture was degassed with nitrogen for 15 min. The mixture was vacuum filtered through a glass frit Buchner funnel with a 20 mm layer of celite. The filter cake was displacement washed with ethanol (2×100 mL) and pulled until slightly ethyl alcohol wet, then wetted with water and the used Raney nickel catalyst was discarded under water. The clear pale amber filtrate was concentrated under reduced pressure to a clear viscous light amber oil. The oil was diluted with methyl tert-butyl ether (1500 mL) and the cloudy solution was concentrated under reduced pressure to a volume of about 150 mL which provided a suspension. The mixture was again diluted with methyl tert-butyl ether (1500 mL) and concentrated under reduced pressure to a volume of about 150 mL. The resulting suspension was allowed to stand at room temperature overnight (about 12 h). The solid was collected by vacuum filtration in a glass frit Buchner funnel and the filter cake was displacement washed with cold methyl tert-butyl ether (2×50 mL) and then pulled for 30 min. The material was further dried in a vacuum oven at 45° C. for 3 h to provide a white solid (19 g, 0.111 mol, 89% yield) as the product, 3-(3-hydroxypropyl)-5,5-dimethyl-pyrrolidin-2-one. ¹H NMR (400 MHz, dimethyl sulfoxide-d₆) δ 7.63 (s, 1H), 3.38 (t, J=6.5 Hz, 2H), 2.37 (tdd, J=9.8, 8.5, 4.4 Hz, 1H), 2.02 (dd, J=12.3, 8.6 Hz, 1H), 1.72 (tdd, J=9.6, 7.5, 4.4 Hz, 1H), 1.52-1.32 (m, 3H), 1.28-1.03 (m, 7H).

### Step 5: (3S)-3-(3-Hydroxypropyl)-5,5-dimethyl-pyrrolidin-2-one

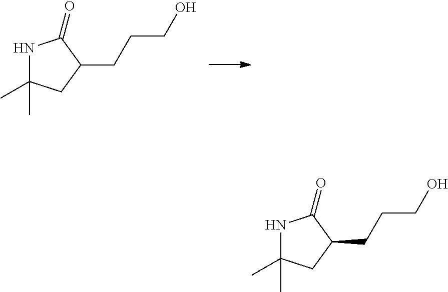

Racemic 3-(3-hydroxypropyl)-5,5-dimethyl-pyrrolidin-2-one (100 g, 566.5 mmol) was separated by chiral SFC chromatography using a ChiralPak AD-H (2×25 cm column) with 30% methanol/carbon dioxide mobile phase at 60 mL/min (injection volume=1 mL of 20 mg/mL solution in methanol giving as the first enantiomer to elute, (3S)-3-(3-hydroxypropyl)-5,5-dimethyl-pyrrolidin-2-one (47 g, 48%) as an off white solid. ¹H NMR (400 MHz, dimethyl sulfoxide-d₆) δ 7.63 (s, 1H), 4.38 (t, J=5.1 Hz, 1H), 3.39 (t, J=5.6 Hz, 2H), 2.37 (ddt, J=13.9, 9.6, 4.4 Hz, 1H), 2.02 (dd, J=12.3, 8.6 Hz, 1H), 1.78-1.64 (m, 1H), 1.42 (td, J=12.8, 12.3, 8.4 Hz, 3H), 1.16 (d, J=17.9 Hz, 7H). ESI-MS m/z calc. 171.12593, found 172.0 (M+1)⁺; Retention time: 0.61 min (LC Method A).

### Step 6: 3-[(3S)-5,5-Dimethylpyrrolidin-3-yl]propan-1-ol

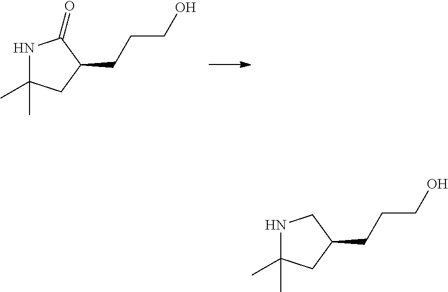

A 5 L, 3-neck round bottom flask was fitted with a mechanical stirrer, a heating mantle, an addition funnel, a J-Kem temperature probe/controller and a nitrogen inlet/outlet. The vessel was charged under a nitrogen atmosphere with lithium aluminum hydride pellets (46.53 g, 1.226 mol) pellets. The vessel was then charged with tetrahydrofuran (500 mL, 20 mL/g). Stirring was commenced and the pot temperature was recorded at 20° C. The mixture was allowed to stir at room temperature for 0.5 h to allow the pellets to dissolve. The pot temperature of the resulting grey suspension was recorded at 24° C. The addition funnel was charged with a solution of (3S)-3-(3-hydroxypropyl)-5,5-dimethyl-pyrrolidin-2-one (60 g, 350.4 mmol) in tetrahydrofuran (600 mL) and the clear pale yellow solution was added dropwise over 90 min. Slight heating was required to get into solution. After the completed addition the pot temperature of the resulting greyish suspension was recorded at 24° C. The mixture was then heated to a pot temperature of 65° C. and the condition was maintained for 72 h. Analysis of the reaction mixture at this point indicated some residual starting material still remaining and no change in product formation. The reaction was subsequently stopped at this point. The heating mantle was removed and the vessel was fitted with a cooling bath. The suspension was cooled to 0° C. with a crushed ice/water cooling bath and then quenched by the very slow dropwise addition of water (46.53 mL), followed by 15 wt % sodium hydroxide solution (46.53 mL) and then finally with water (139.59 mL). The pot temperature of the resulting white suspension was recorded at 5° C. The cooling bath was removed and the vessel was again fitted with a heating mantle. The suspension was warmed to 60° C. and the condition was maintained for 30 min. The warm suspension was vacuum filtered through a glass frit Buchner funnel with a 25 mm layer of celite. The filter cake was then displacement washed with 60° C. tetrahydrofuran (2×350 mL) and then pulled for 30 min. The clear filtrate was concentrated under reduced pressure to provide (55 g, 0.349 mol, 99% yield) of a clear light yellow viscous oil as the desired product, 3-[(3S)-5,5-dimethylpyrrolidin-3-yl]propan-1-ol (55 g, 100%). ¹H NMR (400 MHz, dimethyl sulfoxide-d₆) δ 3.36 (t, J=6.3 Hz, 3H), 2.95 (dd, J=10.6, 7.6 Hz, 1H), 2.40 (dd, J=10.6, 7.7 Hz, 1H), 2.12-1.97 (m, 1H), 1.69 (dd, J=12.1, 8.2 Hz, 1H), 1.47-1.25 (m, 5H), 1.08 (s, 3H), 1.02 (s, 3H).

### Step 7: tert-Butyl (4S)-4-(3-hydroxypropyl)-2,2-dimethyl-pyrrolidine-1-carboxylate

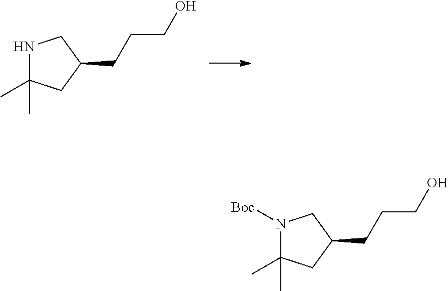 [SMILES: CC1(C)C[C@H](CCCO)CN1 ; CC(C)(C)OC(=O)N1C[C@@H](CCCO)CC1(C)C]

A 1 L, 3-neck round bottom flask was fitted with a mechanical stirrer, a cooling bath, an addition funnel, a J-Kem temperature probe and a nitrogen inlet/outlet. The vessel was charged under a nitrogen atmosphere with 3-[(3S)-5,5-dimethylpyrrolidin-3-yl]propan-1-ol (25 g, 159.0 mmol) and dichloromethane (250 mL) which provided a clear light yellow solution. Stirring was commenced and the pot temperature was recorded at 19° C. The cooling bath was charged with crushed ice/water and the pot temperature was lowered to 0° C. The addition funnel was charged with triethylamine (22.16 mL, 159.0 mmol) which was subsequently added neat dropwise over 5 min. No exotherm was observed. The addition funnel was then charged with di-tert-butyl dicarbonate (31.32 g, 143.5 mmol) dissolved in dichloromethane (150 mL). The clear pale yellow solution was then added dropwise over 30 min which resulted in gentle gas evolution. No exotherm was observed. The cooling bath was removed and the resulting clear light yellow solution was allowed to warm to room temperature and continue to stir at room temperature for 3 h. The reaction mixture was transferred to a separatory funnel and partitioned with water (75 mL). The organic was removed and washed with saturated sodium chloride solution (75 mL), dried over sodium sulfate (150 g) and then filtered through a glass frit Buchner funnel. The filtrate was concentrated under reduced pressure to provide (45 g) of a clear light yellow oil as the desired crude product. The material was purified by silica gel column flash chromatography (liquid load with dichloromethane) eluting with a gradient of 100% dichloromethane to 10% methyl alcohol in dichloromethane over 60 min collecting 50 mL fractions. The desired product fractions were combined and concentrated under reduced pressure to provide tert-butyl (4S)-4-(3-hydroxypropyl)-2,2-dimethyl-pyrrolidine-1-carboxylate (39 g, 95%). ¹H NMR (400 MHz, dimethyl sulfoxide-d₆) δ 4.35 (t, J=5.2 Hz, 1H), 3.54 (dt, J=12.2, 6.3 Hz, 1H), 3.38 (t, J=5.8 Hz, 2H), 2.76 (q, J=10.2 Hz, 1H), 1.47 (s, 3H), 1.44-1.28 (m, 18H), 1.24 (s, 3H). ESI-MS m/z calc. 257.1991, found 258.1 (M+1)⁺; Retention time: 1.55 min (LC Method A).

### Example 4: Preparation of tert-Butyl (4S)-2,2-dimethyl-4-[3-[(6-sulfamoyl-2-pyridyl)amino]propyl]pyrrolidine-1-carboxylate

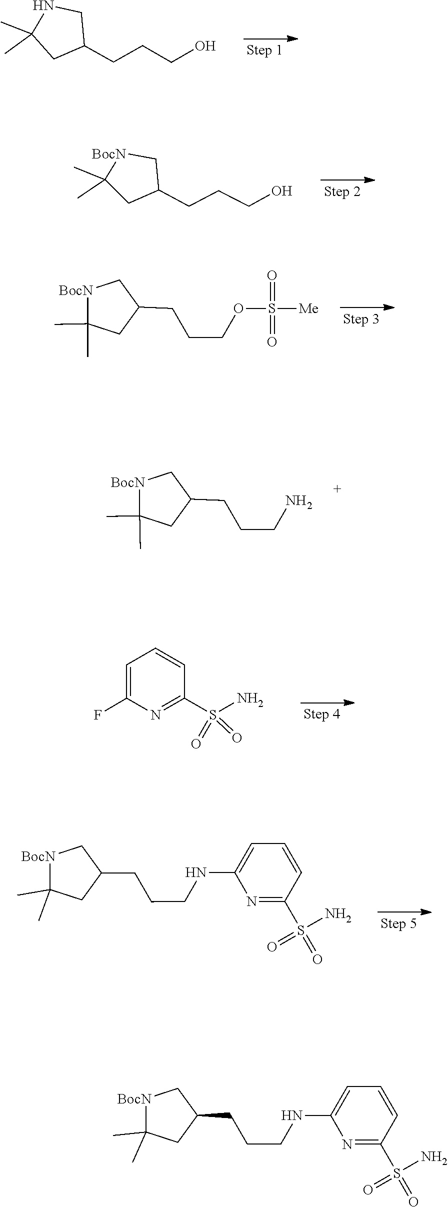

### Step 1: tert-Butyl 4-(3-hydroxypropyl)-2,2-dimethyl-pyrrolidine-1-carboxylate

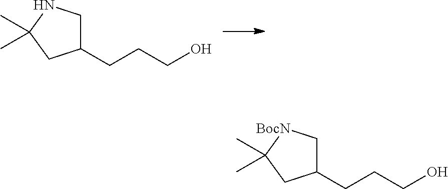 [SMILES: CC1(C)CC(CCCO)CN1 ; CC(C)(C)OC(=O)N1CC(CCCO)CC1(C)C]

A 1 L, 3-neck round bottom flask was fitted with a mechanical stirrer, a cooling bath, an addition funnel, a J-Kem temperature probe and a nitrogen inlet/outlet. The vessel was charged under a nitrogen atmosphere with 3-(5,5-dimethylpyrrolidin-3-yl)propan-1-ol (15 g, 95.39 mmol) and dichloromethane (225 mL, 15 mL/g) which provided a clear light yellow solution. Stirring was commenced and the pot temperature was recorded at 19° C. The cooling bath was charged with crushed ice/water and the pot temperature was lowered to 0° C. The addition funnel was charged with triethylamine (12.55 g, 124.0 mmol) which was subsequently added neat dropwise over 5 min. No exotherm was observed. The addition funnel was then charged with di-tert-butyl dicarbonate (22.89 g, 104.9 mmol) dissolved in dichloromethane (225 mL). The clear pale yellow solution was then added dropwise over 30 min which resulted in gentle gas evolution. No exotherm was observed. The cooling bath was removed and the resulting clear light yellow solution was allowed to warm to room temperature and continue to stir at room temperature for 3 h. The reaction mixture was transferred to a separatory funnel and partitioned with water (75 mL). The organic was removed and washed with saturated sodium chloride solution (75 mL), dried over sodium sulfate (150 g) and then filtered through a glass frit Buchner funnel. The filtrate was concentrated under reduced pressure to provide (30 g) of a clear light yellow oil as the desired crude product. The material was purified by silica gel column flash chromatography (liquid load with dichloromethane) eluting with a gradient of 100% dichloromethane to 10% methyl alcohol in dichloromethane over 60 min collecting 50 mL fractions. The desired product fractions were combined and concentrated under reduced pressure to provide tert-butyl 4-(3-hydroxypropyl)-2,2-dimethyl-pyrrolidine-1-carboxylate (22 g, 0.0855 mol, 90% yield) as a clear pale yellow viscous oil. ¹H NMR (400 MHz, DMSO-d₆) δ 4.38 (td, J=5.2, 1.4 Hz, 1H), 3.54 (dt, J=10.3, 6.7 Hz, 1H), 3.38 (td, J=6.6, 3.5 Hz, 2H), 2.76 (q, J=10.3 Hz, 1H), 2.07 (td, J=11.6, 5.7 Hz, 1H), 1.87 (ddd, J=16.7, 12.1, 6.0 Hz, 1H), 1.37 (dd, J=14.2, 10.4 Hz, 17H), 1.24 (s, 3H).

### Step 2: tert-Butyl 2,2-dimethyl-4-(3-methylsulfonyl oxypropyl)pyrrolidine-1-carboxylate

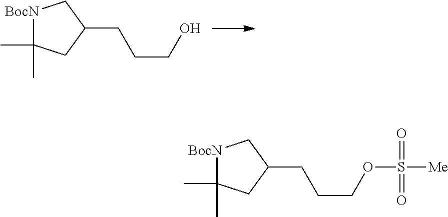 [SMILES: CC(C)(C)OC(=O)N1CC(CCCO)CC1(C)C ; CC(C)(C)OC(=O)N1CC(CCCOS(C)(=O)=O)CC1(C)C]

tert-Butyl 4-(3-hydroxypropyl)-2,2-dimethyl-pyrrolidine-1-carboxylate (50.5 g, 196.22 mmol) and triethylamine (39.711 g, 54.698 mL, 392.44 mmol) were dissolved in dichloromethane (500 mL) and the resulting solution was cooled in an ice water bath for 30 min. Mesyl chloride (24.725 g, 16.706 mL, 215.84 mmol) was added dropwise over a 30 min period, then the ice bath was removed and the mixture stirred at room temperature for one hour. The reaction was then quenched with saturated sodium bicarbonate solution (200 mL). The phases were separated and the organic phase was extracted with saturated sodium bicarbonate (200 mL) and water (2×100 mL). The aqueous phases were discarded and the organic phase was dried over sodium sulfate, filtered and concentrated in vacuo to obtain tert-butyl 2,2-dimethyl-4-(3-methylsulfonyl oxypropyl)pyrrolidine-1-carboxylate (64.2 g, 93%) as a pale yellow oil. ESI-MS m/z calc. 335.1766, found 336.4 (M+1)⁺; Retention time: 5.54 min (LC Method C).

### Step 3: tert-Butyl 4-(3-aminopropyl)-2,2-dimethyl-pyrrolidine-1-carboxylate

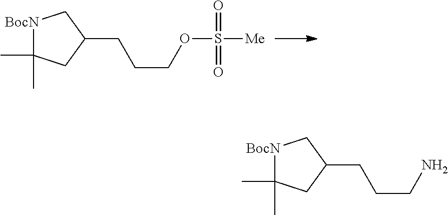 [SMILES: CC(C)(C)OC(=O)N1CC(CCCOS(C)(=O)=O)CC1(C)C ; CC(C)(C)OC(=O)N1CC(CCCN)CC1(C)C]

tert-Butyl 2,2-dimethyl-4-(3-methylsulfonyloxypropyl)pyrrolidine-1-carboxylate (64.2 g, 191.38 mmol) was dissolved in dioxane (650 mL) and then ammonium hydroxide (650 mL) was added and the resulting mixture heated to 45° C. for 18 h. After 18 h, the reaction was cooled to room temperature. The solution was diluted with 1M sodium hydroxide (200 mL) and then extracted with diethyl ether (3×650 mL). The aqueous phase was discarded and the combined organic phases were extracted with water (2×200 mL). The aqueous phases were discarded and the organic phase was dried over sodium sulfate, filtered and concentrated in vacuo to afford tert-butyl 4-(3-aminopropyl)-2,2-dimethyl-pyrrolidine-1-carboxylate (48.9 g, 95%) as a pale yellow oil. ESI-MS m/z calc. 256.2151, found 257.3 (M+1)⁺; Retention time: 3.70 min (LC Method C).

### Step 4: tert-Butyl 2,2-dimethyl-4-[3-[(6-sulfamoyl-2-pyridyl)amino]propyl]pyrrolidine-1-carboxylate

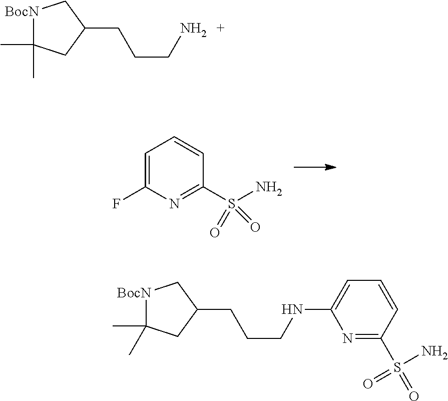 [SMILES: CC(C)(C)OC(=O)N1CC(CCCN)CC1(C)C ; NS(=O)(=O)c1cccc(F)n1 ; CC(C)(C)OC(=O)N1CC(CCCNc2cccc(S(N)(=O)=O)n2)CC1(C)C]

To tert-butyl 4-(3-aminopropyl)-2,2-dimethyl-pyrrolidine-1-carboxylate (8.91 g, 34.8 mmol) and 6-fluoropyridine-2-sulfonamide (6.13 g, 34.8 mmol) in dimethyl sulfoxide (75 mL) was added potassium carbonate (4.91 g, 35.5 mmol) and the mixture stirred at 100° C. for 12 h and then allowed to cool to ambient temperature and stirred for an additional 4 h (16 h total). The reaction mixture was slowly poured into hydrochloric acid (35 mL of 1 M, 35.00 mmol) in water (200 mL) (some foaming) and diluted with ethyl acetate (250 mL). The organic phase was separated and washed with 100 mL of brine. The organic phase was dried over magnesium sulfate, filtered over celite, and concentrated in vacuo to afford a dark yellow oil. The crude product was purified by silica gel chromatography eluting with 0%-100% ethyl acetate in hexanes. Collected both pure (9.0 g) and impure (3 g) fractions. Purified the impure fractions by silica gel chromatography eluting with 0%-100% ethyl acetate in hexanes affording, in total, tert-butyl 2,2-dimethyl-4-[3-[(6-sulfamoyl-2-pyridyl)amino]propyl]pyrrolidine-1-carboxylate (10.0 g, 69%). ¹H NMR (400 MHz, dimethyl sulfoxide-d₆) δ 7.52 (dd, J=8.5, 7.2 Hz, 1H), 7.07 (s, 2H), 6.95 (dd, J=7.2, 0.7 Hz, 2H), 6.61 (d, J=8.5 Hz, 1H), 3.55 (q, J=9.1 Hz, 1H), 3.32-3.24 (m, 2H), 2.79 (q, J=10.0 Hz, 1H), 2.13 (d, J=16.1 Hz, 1H), 1.96-1.82 (m, 1H), 1.51 (dt, J=18.0, 9.3 Hz, 2H), 1.37 (dd, J=12.9, 10.6 Hz, 15H), 1.24 (s, 3H). ESI-MS m/z calc. 412.21442, found 413.1 (M+1)⁺; Retention time: 2.34 min (LC Method K).

### Step 5: tert-Butyl (4S)-2,2-dimethyl-4-[3-[(6-sulfamoyl-2-pyridyl)amino]propyl]pyrrolidine-1-carboxylate

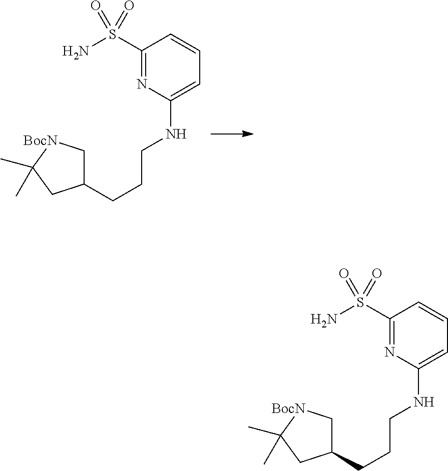 [SMILES: CC(C)(C)OC(=O)N1CC(CCCNc2cccc(S(N)(=O)=O)n2)CC1(C)C ; CC(C)(C)OC(=O)N1C[C@@H](CCCNc2cccc(S(N)(=O)=O)n2)CC1(C)C]

Subjected racemic tert-butyl 2,2-dimethyl-4-[3-[(6-sulfamoyl-2-pyridyl)amino]propyl]pyrrolidine-1-carboxylate (7 g, 16.97 mmol) to chiral separation by SFC chromatography using a ChiralPak IG (250×21.2 mm column, 5 μm particle size) with 40% methanol/60% carbon dioxide mobile phase at 70 mL/min over 11.0 min (injection volume=500 μL of 32 mg/mL solution in methanol) giving as the first peak to elute, tert-butyl(4S)-2,2-dimethyl-4-[3-[(6-sulfamoyl-2-pyridyl)amino]propyl]pyrrolidine-1-carboxylate (3.4481 g, 99%). ESI-MS m/z calc. 412.21442, found 413.2 (M+1)⁺; Retention time: 0.63 min (LC Method J).

### Example 5: Preparation of 3-Diethoxyphosphoryl-1-[(4-methoxyphenyl)methyl]-5,5-dimethyl-pyrrolidin-2-one

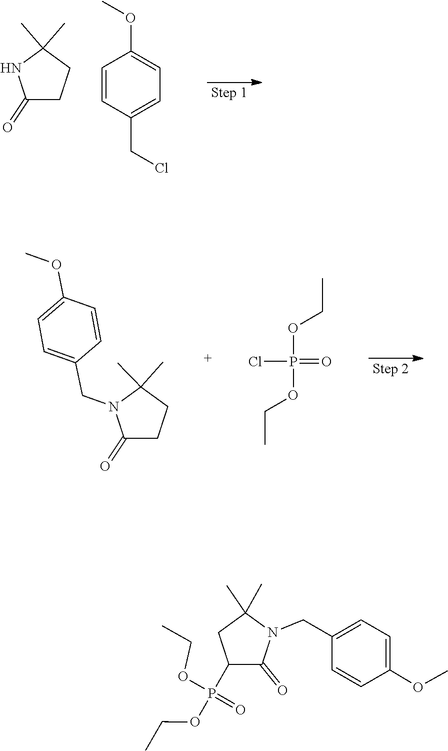

### Step 1: 1-[(4-Methoxyphenyl)methyl]-5,5-dimethyl-pyrrolidin-2-one

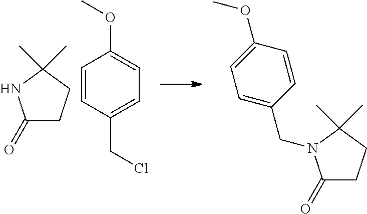

The reaction was run in two batches.

Batch 1: A solution of 5,5-dimethylpyrrolidin-2-one (121 g, 1.069 mol) in DMF (1.8 L) was cooled to 3° C. in an ice water bath, then 60% NaH in mineral oil (64.150 g, 1.604 mol) was added in portions over the course of approximately thirty minutes. The mixture was stirred in the ice water bath for an additional thirty minutes, then at room temperature for 1.5 h. The resulting off-white slurry was again cooled to 3° C. in an ice water bath, then 1-(chloromethyl)-4-methoxy-benzene (251.19 g, 1.604 mol) was added. The ice water bath was removed after the addition and the mixture was stirred overnight. The reaction mixture was carefully quenched with water (100 mL) and stored in a freezer overnight. The mixture was combined with a second batch of material before workup and purification.

Batch 2: A solution of 5,5-dimethylpyrrolidin-2-one (484 g, 4.277 mol) in DMF (7.2 L) was cooled to 3° C. in a 20 L jacketed reactor, then 60% NaH in mineral oil (256.61 g, 6.416 mol) was added in portions over a four hour period. The resulting mixture was stirred at 3° C. for an additional one hour, then at room temperature for two h. The resulting slurry was again cooled to 3° C., then 1-(chloromethyl)-4-methoxy-benzene (1.0048 kg, 6.416 mol) was added. The cooling system was deactivated after the addition was completed and the resulting slurry was stirred overnight while being allowed to warm to room temperature. The reaction mixture was carefully quenched with water (500 mL), combined with the Batch 1 reaction mixture, and split into two equal portions. Each portion was processed as follows: It was diluted with saturated ammonium chloride (10 L), then extracted with ethyl acetate (4×2 L). The aqueous phase was discarded, and then the combined organic phases were diluted with hexane (2 L) and extracted with saturated ammonium chloride (2×2 L), then with water (2 L). The aqueous phases were discarded and the organic phase was dried over sodium sulfate. The combined organic phases from both workups were concentrated in vacuo to obtain crude 1-[(4-methoxyphenyl)methyl]-5,5-dimethyl-pyrrolidin-2-one as a brown oil (1.5 kg). One kilogram of the crude product was purified by silica gel chromatography (0-100% ethyl acetate:hexane) to obtain pure 1-[(4-methoxyphenyl)methyl]-5,5-dimethyl-pyrrolidin-2-one (608 g, 58%) as a yellow oil. ESI-MS m/z calc. 233.1416, found 234.3 (M+1)⁺; Retention time: 3.97 min (LC method C).

### Step 2: 3-Diethoxyphosphoryl-1-[(4-methoxyphenyl)methyl]-5,5-dimethyl-pyrrolidin-2-one

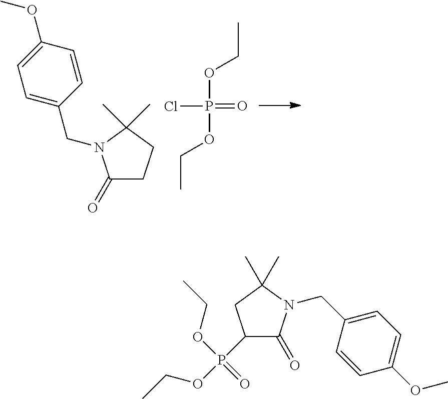

A solution of diisopropylamine (529.14 g, 732.88 mL, 5.23 mol) in tetrahydrofuran (3.5 L) was cooled to −75° C. in a dry ice-acetone bath, then n-butyllithium (2.092 L of 2.5 M in hexanes, 5.2292 mol) was added in a slow stream. The temperature was kept below −70° C. during the course of the addition. The resulting yellow solution was stirred for thirty minutes, then a solution of 1-[(4-methoxyphenyl) methyl]-5,5-dimethyl-pyrrolidin-2-one (488 g, 2.0917 mol) in tetrahydrofuran (1.5 L) was added in a slow stream and then stirred for an additional one hour. The temperature was not allowed to exceed −70° C. during the course of the addition. 1-[Chloro (ethoxy)phosphoryl]oxyethane (541.38 g, 451.15 mL, 3.1376 mol) was added dropwise to the reaction mixture, ensuring that the temperature remained below −70° C. during the course of the addition. The resulting mixture was stirred for sixteen h while being allowed to thaw to room temperature. The reaction was then quenched with 1M hydrochloric acid (3 L) and extracted with ethyl acetate (2×2.4 L). The aqueous phase was discarded and the combined organic phases were extracted with water (3×2.4 L). The aqueous phases were discarded and the organic phase was dried over sodium sulfate and purified by silica gel chromatography (0-100% ethyl acetate/hexane) and then concentrated in vacuo to obtain 3-diethoxyphosphoryl-1-[(4-methoxyphenyl)methyl]-5,5-dimethyl-pyrrolidin-2-one (677 g, 81%). ESI-MS m/z calc. 369.1705, found 370.1 (M+1)⁺; Retention time: 4.2 min (LC method C).

### Example 6: Preparation of 3,3-dicyclopropylpropan-1-ol

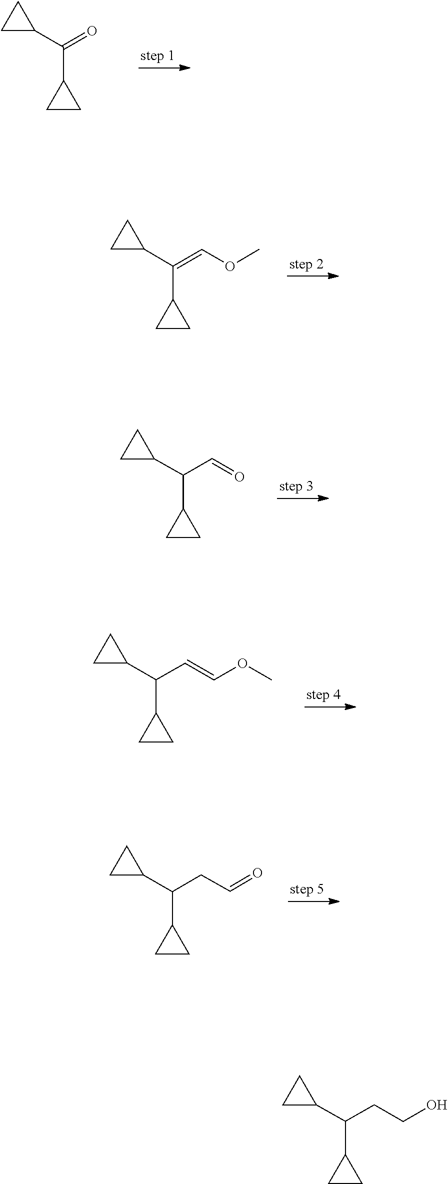

### Step-1: (1-Cyclopropyl-2-methoxy-vinyl)cyclopropane

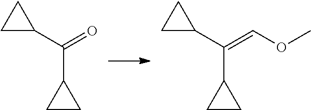

A 5000 mL round bottom flask was fitted with a mechanical stirrer, a cooling bath used as secondary containment, a J-Kem temperature probe, an addition funnel, water cooled reflux condenser and a nitrogen inlet/outlet. The vessel was charged under a nitrogen atmosphere with (methoxymethyl)triphenylphosphonium chloride (97.3 g, 284 mmol) and tetrahydrofuran (375 mL) which provided a white suspension. Stirring was commenced and the pot temperature was recorded at 19° C. The vessel was then charged with potassium tert-butoxide (31.85 g, 0.2838 mol) added as a solid in portions over 10 min which resulted in a reddish orange solution and an exotherm to 21° C. The mixture was continued to stir at room temperature for 30 min. The addition funnel was charged with dicyclopropyl ketone (dicyclopropylmethanone) (25.0 g, 227.0 mmol) which was subsequently added neat, dropwise over 25 min which resulted in a gradual exotherm to 35° C. The resulting reddish orange solution was allowed to gradually cool to room temperature and then continued to stir at room temperature for 4 h. The reaction was then quenched with cold water (375 mL) added dropwise over 25 min. The resulting biphasic mixture was transferred to a separatory funnel and allowed to stand for 5 min. The aqueous was drained and the remaining organic was washed with saturated sodium chloride solution (375 mL). The organic was removed and concentrated under reduced pressure to provide pale yellow oil which still contained some water. The mixture was diluted with ethyl acetate (500 mL) and then transferred to a separatory funnel and partitioned with water (150 mL). The organic was removed, dried over sodium sulfate (150 g) and then filtered through a glass frit Buchner funnel. The filtrate was concentrated under reduced pressure to provide a pale yellow oil with some suspended solids (triphenylphosphine oxide). The mixture was diluted with hexane (500 mL) and then filtered through a glass frit Buchner funnel with a 40 mm layer of silica gel. The filter cake was displacement washed with hexane (2×500 mL). The filtrate was concentrated under reduced pressure to provide (1-cyclopropyl-2-methoxy-vinyl)cyclopropane as a clear pale yellow oil (27 g, 0.1953 mol, 86% yield). ESI-MS m/z calc. 138.10446, found 138.0 (M+1)⁺; Retention time: 1.73 min (LC Method B).

### Step-2: 2,2-Dicyclopropylacetaldehyde

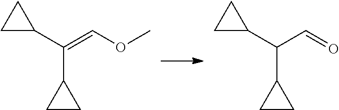

To a solution of (1-cyclopropyl-2-methoxy-vinyl)cyclopropane (128 g, 709.4 mmol) in tetrahydrofuran (700 mL) was added aqueous hydrochloric acid (250 mL of 3 M, 750.0 mmol) and the mixture was stirred at ambient temperature for 16 h then stirred at 55° C. for 4 h and then allowed to cool to ambient temperature over 12 h. The mixture was diluted with 500 mL of brine and the aqueous phase was separated. The aqueous phase was extracted with 500 mL of MTBE and the organic phases were combined. The organic phases were washed with 500 mL of brine, dried over magnesium sulfate, filtered and concentrated in vacuo. The resulting oil was diluted with 250 mL of MTBE and the residual water was removed using a separatory funnel. The organic phase was dried over magnesium sulfate, filtered and concentrated in vacuo affording 2,2-dicyclopropylacetaldehyde (99.2 g, 96%) as a light orange oil. ¹H NMR (400 MHz, Chloroform-d) δ 9.74 (d, J=2.9 Hz, 1H), 1.06 (td, J=8.9, 2.9 Hz, 1H), 0.94-0.81 (m, 2H), 0.64-0.49 (m, 4H), 0.32-0.20 (m, 4H).

### Step-3: [(E)-1-Cyclopropyl-3-methoxy-allyl]cyclopropane

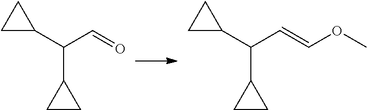

A 5000 mL round bottom flask was fitted with a mechanical stirrer, a heating mantle, a J-Kem temperature probe, an addition funnel, a water cooled reflux condenser and a nitrogen inlet/outlet. The vessel was charged under a nitrogen atmosphere with (methoxymethyl)triphenylphosphonium chloride (116.8 g, 340.7 mmol) and tetrahydrofuran (423 mL) which provided a white suspension. Stirring was commenced and the pot temperature was recorded at 19° C. The vessel was then charged with potassium tert-butoxide (38.22 g, 340.6 mmol) added as a solid in portions over 30 min (12.74 g portion added every 10 min) which resulted in a reddish orange solution and an exotherm to 40° C. The mixture was continued to stir at room temperature for 30 min. The pot temperature was recorded at 36° C. at this point. The addition funnel was then charged with 2,2-dicyclopropylacetaldehyde (47 g of 60% w/w contaminated with triphenylphosphine oxide from previous step, 227.1 mmol) which was subsequently added neat dropwise over 25 min which resulted in a gradual exotherm to 47° C. The resulting reddish orange solution was allowed to gradually cool to room temperature and then continued to stir at room temperature for 15 h. The reaction mixture (pot temp=19° C.) was then quenched with cold water (1000 mL) which resulted in an exotherm to 22° C. The mixture was continued to stir for 10 min. The resulting biphasic mixture was transferred to a separatory funnel and allowed to stand for 5 min. The organic was removed and the residual aqueous was extracted with ethyl acetate (2×300 mL). The combined organic layers were concentrated under reduced pressure to provide a dark amber oil which still contained some water. The mixture was diluted with ethyl acetate (500 mL) and then transferred to a separatory funnel and partitioned with water (150 mL). The organic was removed, washed with saturated sodium chloride solution (200 mL), dried over sodium sulfate (200 g) and then filtered through a glass frit Buchner funnel. The filtrate was concentrated under reduced pressure to provide pale amber oil with some suspended solids (triphenylphosphine oxide). The mixture was diluted with heptane (500 mL) and then allowed to stand at room temperature for 30 min. The suspension was filtered through a glass frit Buchner funnel and the filter cake was displacement washed with heptane (2×100 mL). The filtrate was concentrated under reduced pressure to a volume of about 200 mL. The pale amber solution was cooled to 0° C. in a crushed ice/water cooling bath for 30 min during which time more solids precipitated. The suspension was filtered through a glass frit Buchner funnel and the filter cake was displacement washed with heptane (2×50 mL). The filtrate was concentrated under reduced pressure to provide [(E)-1-cyclopropyl-3-methoxy-allyl]cyclopropane (30 g, 87%). ¹H NMR (400 MHz, Chloroform-d) δ 6.31 (dd, J=12.7, 1.1 Hz, 1H), 4.68 (dd, J 12.7, 7.6 Hz, 1H), 3.51 (s, 3H), 0.77 (qt, J=8.1, 5.0 Hz, 2H), 0.54-0.32 (m, 2H), 0.28-0.12 (m, 5H), 0.08 (ddd, J=9.3, 5.3, 4.1 Hz, 2H).

### Step-4: 3,3-Dicyclopropylpropanal

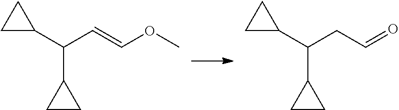

To a solution of [(E)-1-cyclopropyl-3-methoxy-allyl]cyclopropane (141 g, 555.7 mmol) in tetrahydrofuran (500 mL) was added aqueous hydrochloric acid (100 mL of 3 M, 300.0 mmol) and the mixture warmed to 50° C. for 2 h. The mixture was cooled to ambient temperature and the tetrahydrofuran removed in vacuo. The residue was diluted with dichloromethane (700 mL) and the aqueous phase separated (slight emulsion). The organic phase was washed with 500 mL of brine, dried over magnesium sulfate and filtered. To the filtrate was added MgCl₂ (50 g, 525.1 mmol) and the mixture was stirred at ambient temperature for 12 h. The slurry was filtered over Celite (blinded the Celite and needed to be scraped off). The filtrate was slightly cloudy and was washed with brine, dried over magnesium sulfate, filtered and concentrated to afford 3,3-dicyclopropylpropanal (76.8 g, 100%), ¹H NMR (400 MHz, Chloroform-d) δ 9.83 (t, J 2.7 Hz, 1H), 2.71-2.39 (m, 2H), 0.73 (ddt, J=5.5, 4.2, 2.9 Hz, 3H), 0.56-0.47 (m, 2H), 0.42 (dddd, J=9.2, 7.6, 4.0, 2.6 Hz, 2H), 0.28-0.21 (m, 2H), 0.11-0.04 (m, 2H).

### Step-5: 3,3-Dicyclopropylpropan-1-ol

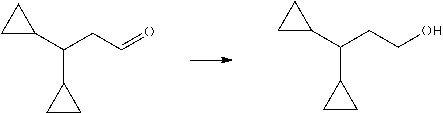 [SMILES: O=CCC(C1CC1)C1CC1 ; OCCC(C1CC1)C1CC1]

To a slurry of lithium aluminum hydride (10.4 g, 266.9 mmol) in tetrahydrofuran (500 mL) was added dropwise a solution of 3,3-dicyclopropylpropanal (76 g, 549.9 mmol) in tetrahydrofuran (150 mL) allowing for a gentle reflux. The mixture was stirred at ambient temperature for 2 h. The reaction was cooled with an ice bath and quenched with the slow addition of water (10.4 mL, 577.3 mmol) followed by aqueous sodium hydroxide (10.4 mL of 4 M, 41.60 mmol), then water (31.2 mL, 1.732 mol). The slurry was filtered over celite, washed with tetrahydrofuran and concentrated in vacuo to afford 3,3-dicyclopropylpropan-1-ol (112 g, 73%), ¹H NMR (400 MHz, Chloroform-d) δ 3.81 (t, J=6.9 Hz, 2H), 1.76 (q, J=6.9 Hz, 2H), 0.63 (dtt, J=8.8, 8.0, 5.1 Hz, 2H), 0.50-0.34 (m, 4H), 0.27-0.14 (m, 3H), 0.14-0.02 (m, 2H).

### Example 7: Preparation of 4-Benzyloxy-6-fluoro-pyridine-2-sulfonamide

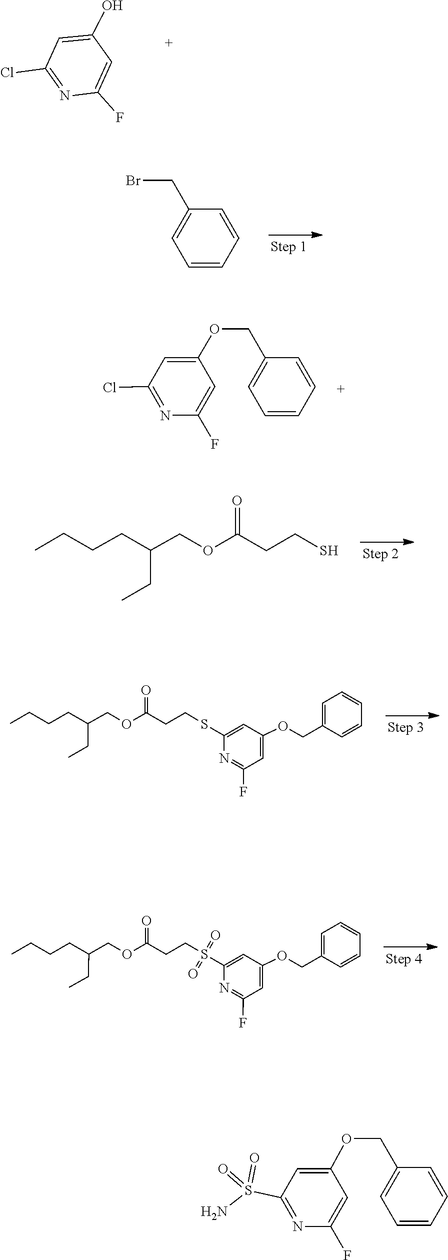

### Step 1: 4-Benzyloxy-2-chloro-6-fluoro-pyridine

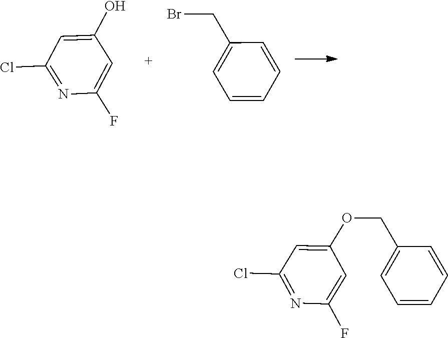 [SMILES: Oc1cc(F)nc(Cl)c1 ; BrCc1ccccc1 ; Fc1cc(OCc2ccccc2)cc(Cl)n1]

To a solution of 2-chloro-6-fluoro-pyridin-4-ol (4.62 g, 31.315 mmol) in acetonitrile (90 mL) was added cesium carbonate (15.3 g, 46.959 mmol) and benzyl bromide (4.1 mL, 34.472 mmol). The reaction was stirred at room temperature overnight. The solution was dissolved in ethyl acetate (350 mL) and water (100 mL). The aqueous phase was removed and the organic phase was washed with water (100 mL) and brine (75 mL). The organic phase was dried over sodium sulfate, filtered and concentrated. The crude was purified by silica gel chromatography eluting with a gradient of 0% to 10% ethyl acetate in heptanes to give 4-benzyloxy-2-chloro-6-fluoro-pyridine (7.01 g, 94%) as a white solid. ESI-MS m/z calc. 237.0357, found 238.1 (M+1)⁺; Retention time: 2.38 min (LC Method N).

### Step 2: 2-Ethylhexyl 3-[(4-benzyloxy-6-fluoro-2-pyridyl)sulfanyl]propanoate

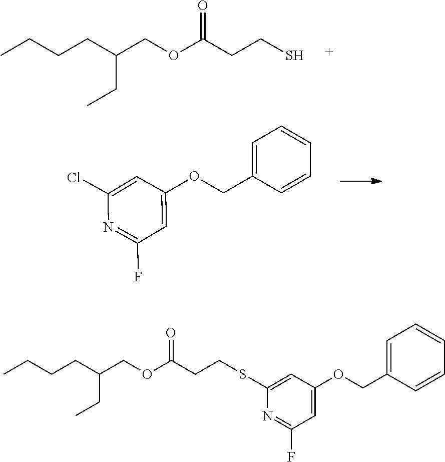 [SMILES: CCCCC(CC)COC(=O)CCS ; Fc1cc(OCc2ccccc2)cc(Cl)n1 ; CCCCC(CC)COC(=O)CCSc1cc(OCc2ccccc2)cc(F)n1]

A solution of 4-benzyloxy-2-chloro-6-fluoro-pyridine (6.77 g, 28.486 mmol) and diisopropylethylamine (7.4200 g, 10 mL, 57.411 mmol) in toluene (250 mL) was degassed by bubbling nitrogen for 5 min. Tris(dibenzylideneacetone)dipalladium(0) (783 mg, 0.8551 mmol), Xantphos (990 mg, 1.7110 mmol) and 2-ethylhexyl 3-sulfanylpropanoate (6.5280 g, 6.8 mL, 29.896 mmol) were added and the mixture was heated at 125° C. overnight. The reaction mixture was concentrated under reduced pressure. The residue was purified by silica gel chromatography using a gradient from 0%-10% of ethyl acetate in heptane to afford 2-ethylhexyl 3-[(4-benzyloxy-6-fluoro-2-pyridyl)sulfanyl]propanoate (12.06 g, quantitative yield) as an orange oil. ¹H NMR (300 MHz, CDCl₃) δ 0.80-0.95 (m, 6H), 1.19-1.43 (m, 8H), 1.51-1.63 (m, 1H), 2.77 (t, J=7.0 Hz, 2H), 3.37 (t, J=6.9 Hz, 2H), 4.02 (dd, J=5.9, 1.2 Hz, 2H), 5.07 (s, 2H), 6.18 (d, J=1.8 Hz, 1H), 6.65 (d, J=1.2 Hz, 1H), 7.28-7.49 (m, 5H). ¹⁹F NMR (282 MHz, CDCl₃) δ −65.7 (s, 1F). ESI-MS m/z calc. 419.193, found 420.2 (M+1)⁺; Retention time: 2.8 min (LC method O).

### Step 3: 2-Ethylhexyl 3-[(4-benzyloxy-6-fluoro-2-pyridyl)sulfonyl]propanoate

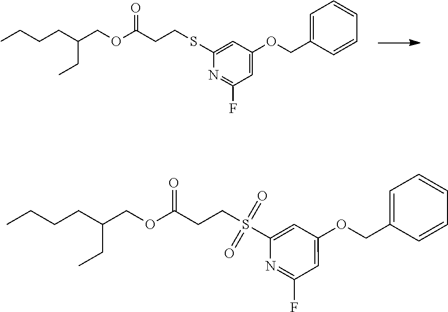 [SMILES: CCCCC(CC)COC(=O)CCSc1cc(OCc2ccccc2)cc(F)n1 ; CCCCC(CC)COC(=O)CCS(=O)(=O)c1cc(OCc2ccccc2)cc(F)n1]

m-Chloroperbenzoic acid (12.9 g, 57.561 mmol) was slowly added at 0° C. to a solution of 2-ethylhexyl 3-[(4-benzyloxy-6-fluoro-2-pyridyl)sulfanyl]propanoate (12.06 g, 28.745 mmol) in dichloromethane (150 mL). The mixture was stirred at this temperature overnight. Ethyl acetate (150 mL) was added and washed with saturated sodium bicarbonate solution (100 mL) and 0.5 M sodium hydroxide solution (2×100 mL). The organic phase was dried over anhydrous sodium sulfate, filtered and concentrated under reduced pressure to afford 2-ethylhexyl 3-[(4-benzyloxy-6-fluoro-2-pyridyl)sulfonyl]propanoate (12.05 g, 93%) as a colorless oil. ESI-MS m/z calc. 451.1829, found 452.2 (M+1)⁺; Retention time: 2.54 min (LC method O).

### Step 4: 4-Benzyloxy-6-fluoro-pyridine-2-sulfonamide

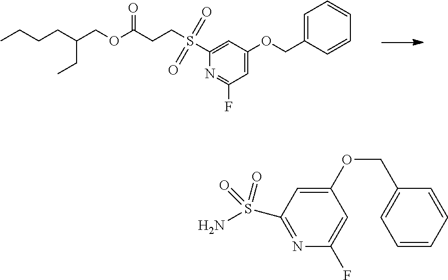 [SMILES: CCCCC(CC)COC(=O)CCS(=O)(=O)c1cc(OCc2ccccc2)cc(F)n1 ; NS(=O)(=O)c1cc(OCc2ccccc2)cc(F)n1]

To a solution of 2-ethylhexyl 3-[(4-benzyloxy-6-fluoro-2-pyridyl)sulfonyl]propanoate (10.69 g, 23.674 mmol) in dimethylsulfoxide (60 mL) was added 1,8-diazabicyclo[5.4.0]undec-7-ene (7.0700 g, 7 mL, 46.441 mmol). The reaction was stirred for 1 h at room temperature and a solution of hydroxylamine-O-sulfonic acid (13.3 g, 117.60 mmol) and sodium acetate (7.7 g, 93.864 mmol) in water (40 mL) was added at 10° C. The reaction was stirred for 1 h at room temperature, diluted with water (300 mL) and extracted with ethyl acetate (2×300 mL). The combined organic layers were washed with brine (300 mL), dried over anhydrous sodium sulfate, filtered and concentrated under reduced pressure. The crude material was triturated overnight with a mix of heptane:ethyl acetate 9:1 (100 mL). The product was isolated by filtration. The product was redissolved in ethyl acetate and filtered over a pad of silica gel, eluting with ethyl acetate (500 mL) and concentrated to give 4-benzyloxy-6-fluoro-pyridine-2-sulfonamide (5.56 g, 83%). ¹H NMR (300 MHz, dimethyl sulfoxide-d₆) δ 5.34 (s, 2H), 7.15 (d, J=2.1 Hz, 1H), 7.30-7.54 (m, 6H), 7.62 (s, 2H). ¹⁹F NMR (282 MHz, dimethyl sulfoxide-d₆) δ −66.0 (s, 1F). ESI-MS m/z calc. 282.0474, found 283.1 (M+1)⁺; Retention time: 1.75 min (LC method O).

### Example 8: Preparation of tert-Butyl 2,2-dimethyl-4-[2-[(6-sulfamoyl-2-pyridyl)amino]ethoxy] pyrrolidine-1-carboxylate

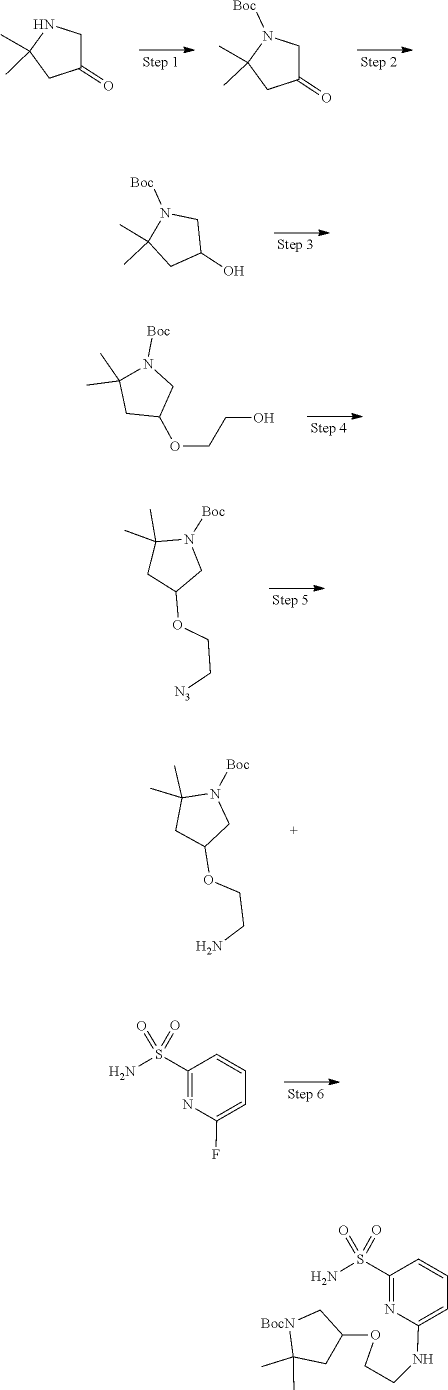

### Step 1: tert-Butyl 2,2-dimethyl-4-oxo-pyrrolidine-1-carboxylate

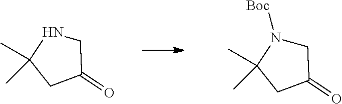

di-tert-Butyl dicarbonate (22.9 g, 24.11 mL, 104.9 mmol) was added to a solution of 5,5-dimethylpyrrolidin-3-one (hydrochloride) (13.08 g, 87.42 mmol), triethylamine (17.71 g, 24.4 mL, 175.0 mmol) and DMAP (1.1 g, 9.004 mmol) in dichloromethane (325 mL) and reaction mixture was stirred at room temperature overnight. The reaction mixture was washed with 1 N hydrochloric acid (300 mL) and the aqueous layer was extracted with dichloromethane (2×250 mL). The organic layers were combined, washed with 5% sodium bicarbonate (250 mL) and brine (150 mL), dried over sodium sulfate, filtered and concentrated under reduced pressure to afford tert-butyl 2,2-dimethyl-4-oxo-pyrrolidine-1-carboxylate (18.5 g, 99%) as a white solid. ¹H NMR (300 MHz, CDCl₃) δ 1.33-1.66 (m, 15H), 2.51 (s, 2H), 3.85 (br. s., 2H). ESI-MS m/z calc. 213.27, found 158.2 (M-C₄H₈)⁺; Retention time: 1.91 min (LC Method 0).

### Step 2: tert-Butyl 4-hydroxy-2,2-dimethyl-pyrrolidine-1-carboxylate

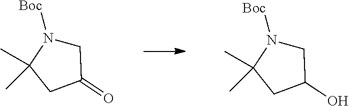

tert-Butyl 2,2-dimethyl-4-oxo-pyrrolidine-1-carboxylate (150 mg, 0.7033 mmol) was dissolved in dry methanol (2.5 mL) and cooled in an ice-bath. NaBH₄ (30 mg, 0.7930 mmol) was added carefully and the reaction mixture was stirred at 0° C. for 1.5 h. The reaction mixture was diluted with water (25 mL), and 1M aqueous hydrochloric acid (0.5 mL). The aqueous layer was extracted with ethyl acetate (2×25 mL) and the organic layers were combined, washed with brine (10 mL) dried with sodium sulfate, filtered and concentrated in vacuo giving tert-butyl 4-hydroxy-2,2-dimethyl-pyrrolidine-1-carboxylate (150 mg, 99%) which was used directly in the ensuing step. ESI-MS m/z calc. 215.15215, found 216.2 (M+1)⁺; Retention time: 0.48 min (LC Method J).

### Step 3: tert-Butyl 4-(2-hydroxyethoxy)-2,2-dimethyl-pyrrolidine-1-carboxylate

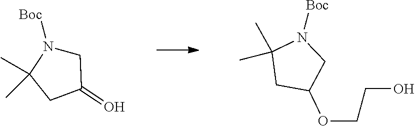 [SMILES: CC(C)(C)OC(=O)N1CC(=O)CC1(C)C ; CC(C)(C)OC(=O)N1CC(OCCO)CC1(C)C]

In a 250 mL flask, tert-butyl 4-hydroxy-2,2-dimethyl-pyrrolidine-1-carboxylate (3 g, 13.93 mmol) was dissolved in N,N-dimethylformamide (15 mL) at 0° C. and sodium hydride (1.8 g of 60% w/w in a mineral oil, 45.00 mmol) was carefully added. The mixture was stirred at 0° C. for 15 min and then 2-bromoethoxy-tert-butyl-dimethyl-silane (9 mL, 41.95 mmol) was added dropwise at 0° C. and the mixture was allowed to warm to room temperature. The mixture was stirred for 16 h. The mixture was then cooled to 0° C., quenched with water and extracted with diethyl ether. The organic extract was washed with water, dried (sodium sulfate), filtered and concentrated in vacuo. The resulting orange oil was purified by silica gel chromatography eluting with a gradient from 0-30% ethyl acetate in hexanes to afford the tert-butyl(dimethyl)silyl protected intermediate which was dissolved in tetrahydrofuran (20 mL), treated with a tetrahydrofuran solution of TBAF (28 mL of 1 M, 28.00 mmol) and stirred for 2 h at room temperature. The mixture was concentrated and the residue was dissolved in dichloromethane, washed with water, dried over sodium sulfate, filtered and evaporated in vacuo. The obtained crude material was purified by silica gel chromatography eluting with a gradient from 0-20% methanol in dichloromethane to give tert-butyl 4-(2-hydroxyethoxy)-2,2-dimethyl-pyrrolidine-1-carboxylate (1.6 g, 44% yield). ¹H NMR (400 MHz, Chloroform-d) δ 3.97 (d, J=4.0 Hz, 1H), 3.80-3.34 (m, 6H), 2.05 (t, J=6.1 Hz, 1H), 1.97 (d, J=16.4 Hz, 2H), 1.52-1.30 (m, 15H). ESI-MS m/z calc. 259.17834, found 260.17 (M+1)⁺; Retention time: 0.55 min (LC Method J).

### Step 4: tert-Butyl 4-(2-azidoethoxy)-2,2-dimethyl-pyrrolidine-1-carboxylate

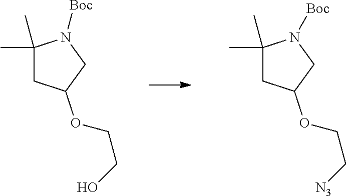 [SMILES: CC(C)(C)OC(=O)N1CC(OCCO)CC1(C)C ; CC(C)(C)OC(=O)N1CC(OCCN=[N+]=[N-])CC1(C)C]

To a solution of tert-butyl 4-(2-hydroxyethoxy)-2,2-dimethyl-pyrrolidine-1-carboxylate (1.6 g, 6.169 mmol) in dichloromethane (20 mL) was added triethylamine (5.2 mL, 37.31 mmol) followed by methanesulfonyl chloride (1.4 mL, 18.09 mmol) at 0° C. The reaction mixture was stirred at room temperature for 20 h. The reaction mixture was quenched with ice-water and dichloromethane and the resulting layers were separated and the organic layer was dried over sodium sulfate, filtered and concentrated in vacuo to afford the crude mesylate which was combined with sodium azide (1.2 g, 18.46 mmol) in N,N-dimethylformamide (10 mL) and the mixture was stirred at 50° C. for 2 h. The reaction was quenched with water and extracted with diethyl ether. The organic extract was dried over sodium sulfate, evaporated in vacuo and the residue was purified by silica gel chromatography eluting with a gradient from 0-50% ethyl acetate in hexanes to give tert-butyl 4-(2-azidoethoxy)-2,2-dimethyl-pyrrolidine-1-carboxylate (1.33 g, 76%). ¹H NMR (400 MHz, Chloroform-d) δ 3.96 (q, J=4.6 Hz, 1H), 3.69-3.31 (m, 6H), 2.06-1.91 (m, 2H), 1.53-1.32 (m, 15H).

### Step 5: tert-Butyl 4-(2-aminoethoxy)-2,2-dimethyl-pyrrolidine-1-carboxylate

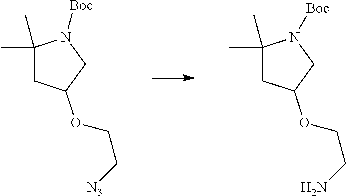 [SMILES: CC(C)(C)OC(=O)N1CC(OCCN=[N+]=[N-])CC1(C)C ; CC(C)(C)OC(=O)N1CC(OCCN)CC1(C)C]

To a solution of tert-butyl 4-(2-azidoethoxy)-2,2-dimethyl-pyrrolidine-1-carboxylate (1.33 g, 4.677 mmol) in methanol (20 mL) was added palladium on carbon (500 mg of 10% w/w, 0.470 mmol). The mixture was saturated with hydrogen gas and stirred at room temperature while sparging hydrogen through the reaction mixture for 2 h. The mixture was filtered and evaporated in vacuo to afford tert-butyl 4-(2-aminoethoxy)-2,2-dimethyl-pyrrolidine-1-carboxylate (1.188 g, 98%). ¹H NMR (400 MHz, Chloroform-d) δ 4.00-3.87 (m, 1H), 3.69-3.35 (m, 4H), 2.94-2.71 (m, 2H), 2.07-1.83 (m, 2H), 1.47 (q, J=9.4, 8.4 Hz, 15H).

### Step 6: tert-Butyl 2,2-dimethyl-4-[2-[(6-sulfamoyl-2-pyridyl)amino]ethoxy]pyrrolidine-1-carboxylate

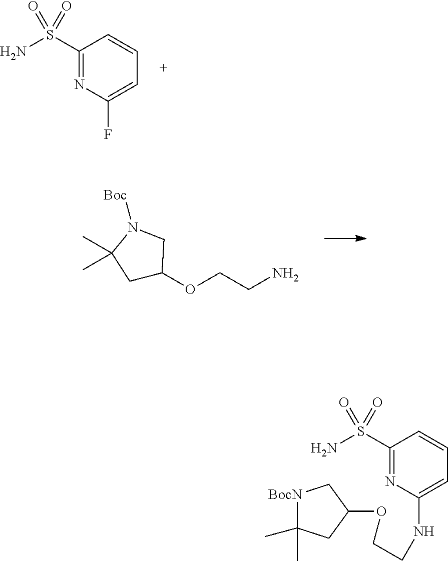 [SMILES: NS(=O)(=O)c1cccc(F)n1 ; CC(C)(C)OC(=O)N1CC(OCCN)CC1(C)C ; CC(C)(C)OC(=O)N1CC(OCCNc2cccc(S(N)(=O)=O)n2)CC1(C)C]

In a sealed 20 mL microwave vial, a solution of tert-butyl 4-(2-aminoethoxy)-2,2-dimethyl-pyrrolidine-1-carboxylate (1.188 g, 4.598 mmol), 6-fluoropyridine-2-sulfonamide (810 mg, 4.598 mmol) and diisopropylethylamine (4 mL, 22.96 mmol) in n-BuOH (10 mL) was stirred at 150° C. for 16 h. The solvent was removed in vacuo and the residue was dissolved in dichloromethane and washed with water. The organic extract was dried over sodium sulfate, evaporated and purified by silica gel chromatography eluting with a gradient from 0-40% ethyl acetate in hexanes to give tert-butyl 2,2-dimethyl-4-[2-[(6-sulfamoyl-2-pyridyl)amino]ethoxy]pyrrolidine-1-carboxylate (1.500 g, 66%). ESI-MS m/z calc. 414.1937, found 415.3 (M+1)⁺; Retention time: 0.61 min (LC Method J).

### Example 9: Preparation of tert-Butyl (4S)-2,2-dimethyl-4-[3-(2-pyridyl)-3-[(6-sulfamoyl-2-pyridyl)amino]propyl]pyrrolidine-1-carboxylate

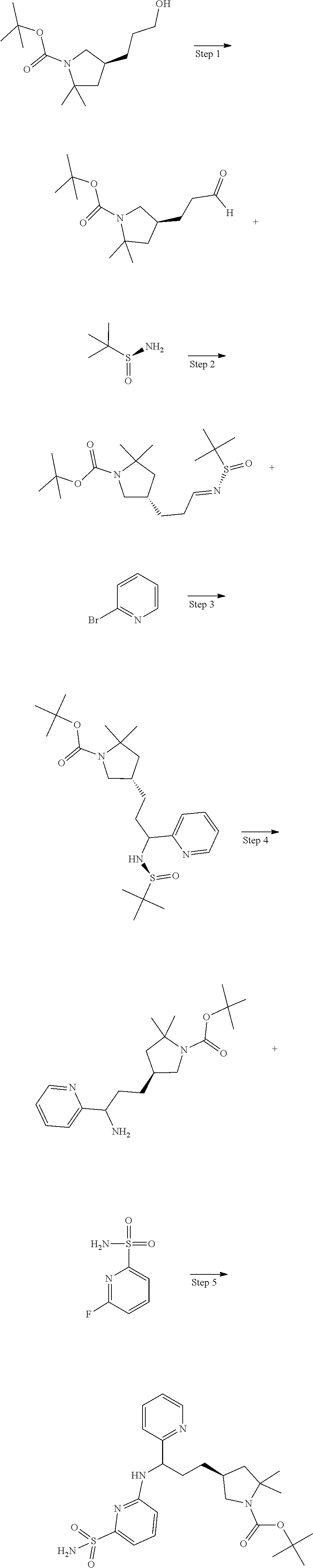

### Step 1: tert-Butyl (4S)-2,2-dimethyl-4-(3-oxopropyl)pyrrolidine-1-carboxylate

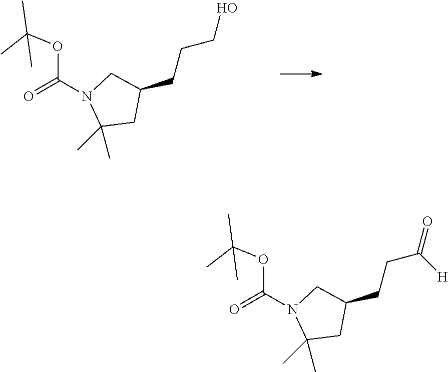 [SMILES: CC(C)(C)OC(=O)N1C[C@@H](CCCO)CC1(C)C ; CC(C)(C)OC(=O)N1C[C@@H](CCC=O)CC1(C)C]

A buffered solution of bleach was prepared by dissolving sodium bicarbonate (5.61 g, 66.78 mmol) into a solution of sodium hypochlorite (1.47 M in water) (87 mL, 127.89 mmol) and that solution was cooled in an ice bath. The solution was then added dropwise to a second solution stirred mechanically, that was prepared in advance by adding sodium bromide (640 mg, 6.22 mmol) (dissolved in water (3.6 mL)) and then TEMPO (42 mg, 0.2688 mmol) to tert-butyl (4S)-4-(3-hydroxypropyl)-2,2-dimethyl-pyrrolidine-1-carboxylate (30 g, 116.56 mmol) in DCM (400 mL) maintained at −13° C. During the addition of the buffered bleach solution to the substrate solution the internal temperature was maintained below −10° C. and after completion of the addition, the reaction mixture was maintain at −14° C. for 15 min. Then the excess bleach was quenched with ethanol (3 mL, 51.38 mmol). The reaction mixture was concentrated under reduced pressure at 25° C. The resulting residue was partitioned between ethyl acetate (300 mL) and water (100 mL). The aqueous phase was separated and washed with ethyl acetate (100 mL). The organics were combined, washed with brine (50 mL), dried with sodium sulfate, filtered and concentrated under reduced pressure to provide the pure tert-butyl (4S)-2,2-dimethyl-4-(3-oxopropyl)pyrrolidine-1-carboxylate (27.9 g, 89%) as a clear oil; ¹H NMR (400 MHz, CDCl₃) δ 9.78 (s, 1H), 3.81-3.57 (m, 1H), 2.99-2.81 (m, 1H), 2.46 (t, J=6.8 Hz, 2H), 2.19-2.04 (m, 1H), 1.89 (td, J=12.2, 5.9 Hz, 1H), 1.75-1.60 (m, 2H), 1.59-1.21 (m, 16H). ESI-MS m/z calc. 255.1834, found 200.2 (M−99)⁺; Retention time: 1.86 minutes (LC method E).

### Step 2: tert-Butyl (4S)-4-[(3E)-3-[(S)-tert-butylsulfinyl]iminopropyl]-2,2-dimethyl-pyrrolidine-1-carboxylate

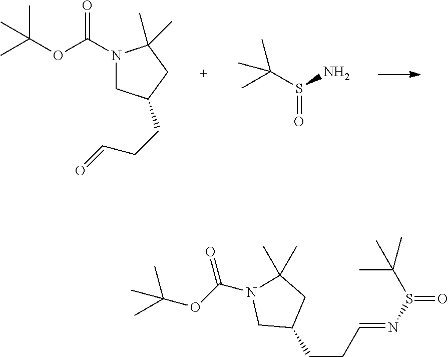

tert-Butyl (4S)-2,2-dimethyl-4-(3-oxopropyl)pyrrolidine-1-carboxylate (12.02 g, 44.72 mmol) was dissolved in DCM (170 mL). (S)-2-Methylpropane-2-sulfinamide (11.10 g, 89.75 mmol), magnesium sulfate (32.96 g, 272.46 mmol) and pyridinium p-toluenesulfonate (6.93 g, 27.02 mmol) were added sequentially. The mixture was vigorously stirred under nitrogen for 18 h at rt, filtered through a celite pad, washed with DCM (2×250 mL). The combined filtrate was concentrated under vacuum. The crude (28.57 g, white-yellow solid) was subjected to flash chromatography (pre-adsorbed to SiO₂) (330 g SiO₂, eluting 0 to 30% EtOAc/hexanes in 65 min.). Appropriate fractions were collected and concentrated under vacuum. tert-Butyl (4S)-4-[(3E)-3-[(S)-tert-butylsulfinyl]iminopropyl]-2,2-dimethyl-pyrrolidine-1-carboxylate (14.8 g, 88%). ESI-MS m/z calc. 358.229, found 359.6 (M+1)⁺; Retention time: 3.23 minutes (LC method B).

### Step 3: tert-Butyl (4S)-4-[3-[[(S)-tert-butylsulfinyl]amino]-3-(2-pyridyl)propyl]-2,2-dimethyl-pyrrolidine-1-carboxylate

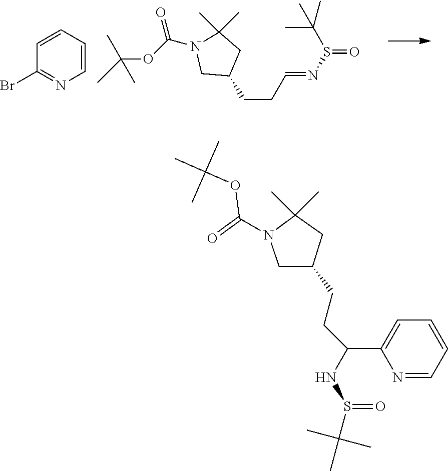

To a mixture of 2-bromopyridine (3.81 g, 2.3 mL, 24.12 mmol) in anhdrous THF (72 mL) stirring vigorously at −78° C. under nitrogen was added dropwise n-butyllithium (9.5 mL of 2.5 M in hexanes, 23.750 mmol). The mixture was stirred vigorously for 0.5 h at −78° C. and a solution of tert-butyl (4S)-4-[(3E)-3-[(S)-tert-butylsulfinyl]iminopropyl]-2,2-dimethyl-pyrrolidine-1-carboxylate (4.50 g, 11.923 mmol) in anhydrous THF (20 mL) was added dropwise. The reaction was stirred for 30 min. at −78° C. The reaction was quenched with saturated aqueous NH₄Cl (200 mL) and was allowed to warm to rt.H₂O (200 mL) was added and the mixture was extracted with EtOAc (3×250 mL). The combined organic layers were washed with saturated aqueous NaCl (200 mL), dried over anhydrous sodium sulfate, filtered and concentrated under vacuum to give a crude material as an orange foamy oil. tert-Butyl (4S)-4-[3-[[(S)-tert-butylsulfinyl]amino]3-(2-pyridyl)propyl]-2,2-dimethyl-pyrrolidine-1-carboxylate (6.1350 g, 106%). ESI-MS m/z calc. 437.2712, found 438.0 (M+1)⁺; Retention time: 2.42 minutes (LC method B).

### Step 4: tert-Butyl (4S)-4-[3-amino-3-(2-pyridyl)propyl]-2,2-dimethyl-pyrrolidine-1-carboxylate

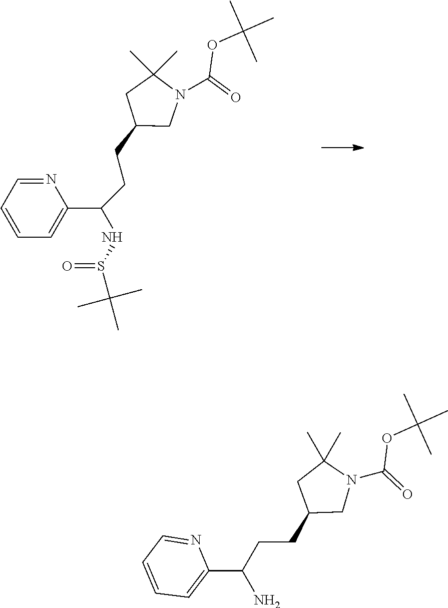 [SMILES: CC(C)(C)OC(=O)N1C[C@@H](CCC(N[S@@](=O)C(C)(C)C)c2ccccn2)CC1(C)C ; CC(C)(C)OC(=O)N1C[C@@H](CCC(N)c2ccccn2)CC1(C)C]

tert-Butyl (4S)-4-[3-[[(S)-tert-butylsulfinyl]amino]-3-(2-pyridyl)propyl]-2,2-dimethyl-pyrrolidine-1-carboxylate (13.5 g, 26.22 mmol) was dissolved in THF (180 mL) and water (36 mL). Molecular iodine (2 g, 7.88 mmol) was added. The mixture was stirred at 35° C. for 16 h. It was then cooled to rt and partitioned between EtOAc (300 mL) and Na₂S₂O₃ (50 g) in saturated aqueous sodium bicarbonate (300 mL). The layers were separated and the aqueous layer was extracted once with EtOAc (200 mL). The organic layer was concentrated and the residue was dissolved in 1M HCl (500 mL) and was extracted with diethyl ether (300 mL). The aqueous layer was basified by 2.5M NaOH and extracted with EtOAc (2×300 mL). The organic layer was washed with brine, dried over anhydrous Na₂SO₄, filtered and concentrated to give tert-butyl (4S)-4-[3-amino-3-(2-pyridyl)propyl]-2,2-dimethyl-pyrrolidine-1-carboxylate (8.5 g, 92%). ESI-MS m/z calc. 333.2416, found 334.6 (M+1)⁺; Retention time: 2.55 minutes (LC method B).

### Step 5: tert-Butyl (4S)-2,2-dimethyl-4-[3-(2-pyridyl)-3-[(6-sulfamoyl-2-pyridyl)amino]propyl]pyrrolidine-1-carboxylate

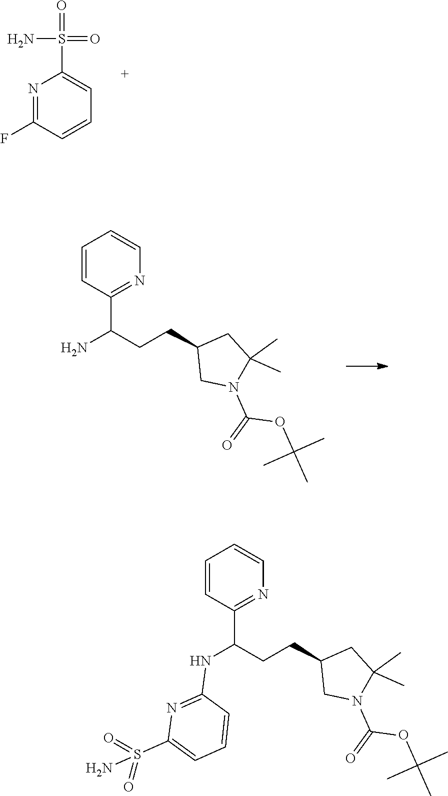 [SMILES: NS(=O)(=O)c1cccc(F)n1 ; CC(C)(C)OC(=O)N1C[C@@H](CCC(N)c2ccccn2)CC1(C)C ; CC(C)(C)OC(=O)N1C[C@@H](CCC(Nc2cccc(S(N)(=O)=O)n2)c2ccccn2)CC1(C)C]

To a mixture of tert-butyl (4S)-4-[3-amino-3-(2-pyridyl)propyl]-2,2-dimethyl-pyrrolidine-1-carboxylate (8.5 g, 24.215 mmol) and 6-fluoropyridine-2-sulfonamide (8 g, 43.140 mmol) in DMSO (22 mL) was added DIEA (12 mL, 68.893 mmol). The mixture was stirred at 115° C. for 20 h. It was then cooled to rt and partitioned between EtOAc (300 mL) and saturated sodium bicarbonate (300 mL). The aqueous layer was extracted with more EtOAc (300 mL). The combined EtOAc solution was washed with brine (2×500 mL), dried over anhydrous Na₂SO₄, filtered and concentrated. The residue was purified by silica gel chromatography, using 5-100% EtOAc in hexanes to afford tert-butyl (4S)-2,2-dimethyl-4-[3-(2-pyridyl)-3-[(6-sulfamoyl-2-pyridyl)amino]propyl]pyrrolidine-1-carboxylate (10.41 g, 83%) as a solid. 1H NMR (500 MHz, DMSO-d6) δ 8.54 (d, J=4.6 Hz, 1H), 7.72 (td, J=7.7, 7.6, 1.8 Hz, 1H), 7.57-7.42 (m, 3H), 7.28-7.20 (m, 1H), 7.06 (s, 2H), 6.96 (d, J=7.2 Hz, 1H), 6.76-6.68 (m, 1H), 5.19 (s, 1H), 3.58-3.48 (m, 1H), 2.83-2.69 (m, 1H), 2.14-2.02 (m, 1H), 1.95-1.78 (m, 3H), 1.42-1.30 (m, 15H), 1.22 (s, 3H). ESI-MS m/z calc. 489.241, found 490.3 (M+1)+; Retention time: 1.82 minutes (LC method H).

### Example 10: Preparation of (14S,17R)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 246 (less polar isomer), and (14S,17S)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 247 (more polar isomer)

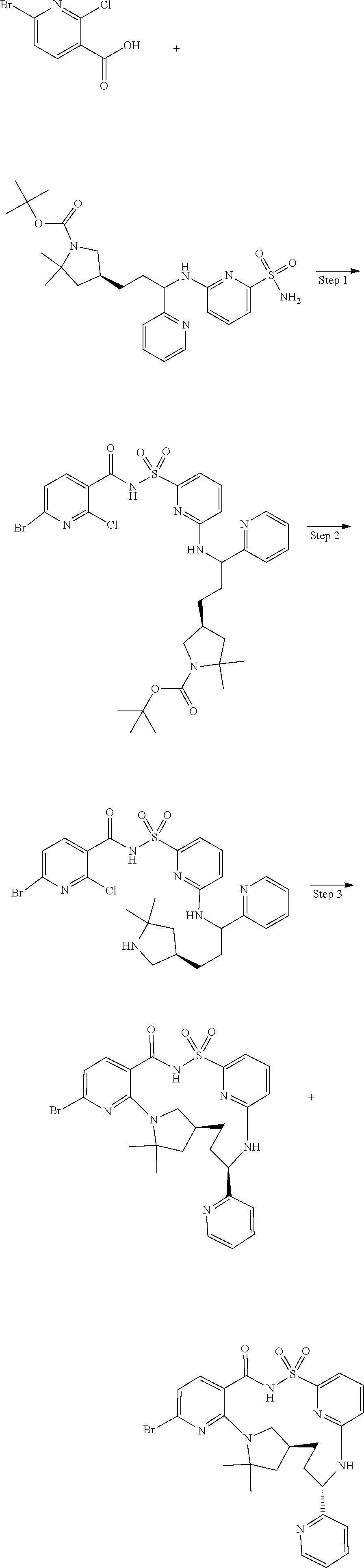 [SMILES: O=C(O)c1ccc(Br)nc1Cl ; CC(C)(C)OC(=O)N1C[C@@H](CCC(Nc2cccc(S(N)(=O)=O)n2)c2ccccn2)CC1(C)C ; CC(C)(C)OC(=O)N1C[C@@H](CCC(Nc2cccc(S(=O)(=O)NC(=O)c3ccc(Br)nc3Cl)n2)c2ccccn2)CC1(C)C ; CC1(C)C[C@@H](CCC(Nc2cccc(S(=O)(=O)NC(=O)c3ccc(Br)nc3Cl)n2)c2ccccn2)CN1 ; CC1(C)C[C@@H]2CC[C@H](c3ccccn3)Nc3cccc(n3)S(=O)(=O)NC(=O)c3ccc(Br)nc3N1C2 ; CC1(C)C[C@@H]2CC[C@@H](c3ccccn3)Nc3cccc(n3)S(=O)(=O)NC(=O)c3ccc(Br)nc3N1C2]

### Step 1: tert-Butyl (4S)-4-[3-[[6-[(6-bromo-2-chloro-pyridine-3-carbonyl)sulfamoyl]-2-pyridyl]amino]-3-(2-pyridyl)propyl]-2,2-dimethyl-pyrrolidine-1-carboxylate

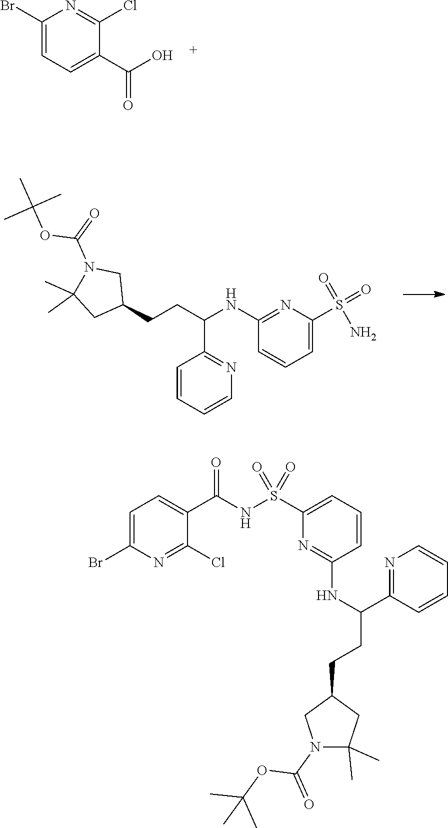 [SMILES: O=C(O)c1ccc(Br)nc1Cl ; CC(C)(C)OC(=O)N1C[C@@H](CCC(Nc2cccc(S(N)(=O)=O)n2)c2ccccn2)CC1(C)C ; CC(C)(C)OC(=O)N1C[C@@H](CCC(Nc2cccc(S(=O)(=O)NC(=O)c3ccc(Br)nc3Cl)n2)c2ccccn2)CC1(C)C]

6-Bromo-2-chloro-pyridine-3-carboxylic acid (2.9 g, 12.26 mmol) and CDI (2.0 g, 12.33 mmol) were dissolved in THF (18 mL) and the mixture stirred at 60° C. for 45 min then tert-butyl (4S)-2,2-dimethyl-4-[3-(2-pyridyl)-3-[(6-sulfamoyl-2-pyridyl)amino]propyl]pyrrolidine-1-carboxylate (3000 mg, 6.127 mmol) was added followed by DBU (4.12 mL, 27.55 mmol) and the resulting mixture was stirred at rt for 2 h. The mixture was diluted with EtOAc and washed with 1N HCl in water, water, brine, dried (MgSO₄), and concentrated. The residue was purified (flash chromatography: 80 g SiO₂, 50-100% EtOAc in hexanes) to provide the desired product tert-butyl (4S)-4-[3-[[6-[(6-bromo-2-chloro-pyridine-3-carbonyl)sulfamoyl]-2-pyridyl]amino]-3-(2-pyridyl)propyl]-2,2-dimethyl-pyrrolidine-1-carboxylate (4.11 g, 95%). ESI-MS m/z calc. 706.134, found 706.9 (M+1)⁺; Retention time: 0.59 minutes (LC method I).

### Step 2: 6-Bromo-2-chloro-N-[[6-[[3-[(3R)-5,5-dimethylpyrrolidin-3-yl]-1-(2-pyridyl)propyl]amino]-2-pyridyl]sulfonyl]pyridine-3-carboxamide

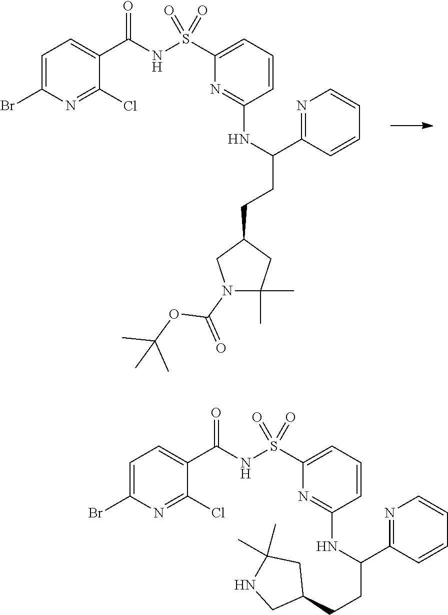 [SMILES: CC(C)(C)OC(=O)N1C[C@@H](CCC(Nc2cccc(S(=O)(=O)NC(=O)c3ccc(Br)nc3Cl)n2)c2ccccn2)CC1(C)C ; CC1(C)C[C@@H](CCC(Nc2cccc(S(=O)(=O)NC(=O)c3ccc(Br)nc3Cl)n2)c2ccccn2)CN1]

To a solution of tert-butyl (4S)-4-[3-[[6-[(6-bromo-2-chloro-pyridine-3-carbonyl)sulfamoyl]-2-pyridyl]amino]-3-(2-pyridyl)propyl]-2,2-dimethyl-pyrrolidine-1-carboxylate (1123 mg, 1.586 mmol) in DCM (7.9 mL) was added TFA (3.7 mL, 48.03 mmol) and the mixture was stirred at rt for 1 h. The solution was concentrated to dryness under reduced pressure, then co-evaporated with toluene (2×5 mL), then dried under high vacuum at rt for 16 h to provide 6-bromo-2-chloro-N-[[6-[[3-[(3R)-5,5-dimethylpyrrolidin-3-yl]-1-(2-pyridyl)propyl]amino]-2-pyridyl]sulfonyl]pyridine-3-carboxamide (trifluoroacetate salt) (1600 mg, 106%). ¹H NMR (500 MHz, DMSO-d₆) δ 8.68 (s, 1H), 8.63 (d, J=5.3 Hz, 1H), 8.02 (t, J=7.8 Hz, 1H), 7.91 (s, 1H), 7.80 (s, 2H), 7.73-7.65 (m, 2H), 7.51 (t, J=6.5 Hz, 1H), 6.93 (d, J=8.5 Hz, 1H), 5.07 (s, 1H), 3.44-3.32 (m, 1H), 2.82 (ddt, J=17.9, 11.4, 6.1 Hz, 1H), 2.39 (dt, J=9.8, 6.1 Hz, 1H), 2.04-1.81 (m, 3H), 1.63-1.39 (m, 2H), 1.36 (d, J=4.3 Hz, 4H), 1.27 (d, J=2.8 Hz, 3H). ESI-MS m/z calc. 606.08154, found 607.0 (M+1)⁺; Retention time: 0.3 minutes (LC method D).

### Step 3: (14S,17R)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 246 (less polar isomer), and (14S,17S)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 247 (more polar isomer)

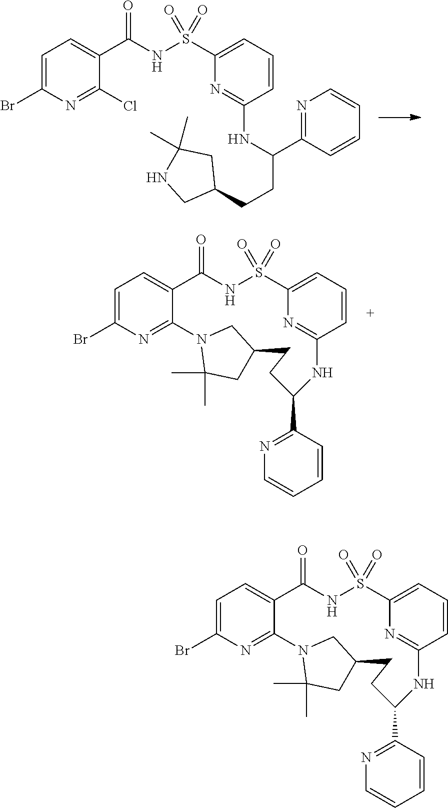

A mixture of 6-bromo-2-chloro-N-[[6-[[3-[(3R)-5,5-dimethylpyrrolidin-3-yl]-1-(2-pyridyl)propyl]amino]-2-pyridyl]sulfonyl]pyridine-3-carboxamide (Trifluoroacetate salt)(3.62 g, 3.810 mmol) and potassium carbonate (2.63 g, 19.03 mmol) in DMA (38 mL) was heated at 140° C. for 5 h, cooled by ice bath, 38 mL of water added and then 38 mL of 1 M HCl which produced a mixture <pH 2. To this foamy mixture was added 10 mL of 1 M sodium bicarbonate which collapsed most of the foam and converted the mixture to pH 7. After allowing the mixture temperature to rise to rt with stirring, the foam had converted to a tan grannular solid. The solid was collected by filtration and dried under suction to provide 700 mg of tan solid. The filtrate was extracted with EtOAc (4×120 mL). The product remained in the aqueous layer and so it was converted to pH 4 with the addition of 3 mL of 1 M HCl. Then it was extracted with EtOAc (2×120 mL). The combined organic extracts were dried (MgSO₄) and evaporated to an oil which was transferred to a 100 mL flask and rotary evaporated at 50 C/4 torr to provide 1.7 g of the product as a gum. Both the solid precipitate and the gum obtained contained ca. a 1:1 product isomer mixture by UPLC. Both samples were dissolved into 1:1 ACN/MeOH with heat at a concentration of 100 mg/mL. These solutions were purified by preparative SFC eluting a gradient of 5 mM NH₃ in methanol to CO₂ (50-80% over 10 min) though a 21.2×250 mm OD-3 column, 5 μm particle, with a series of 333 μL injections giving as a white solid (after evaporation of the collected fractions), first the less polar isomer (14S,17R)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (546 mg, 25%). ¹H NMR (400 MHz, Chloroform-d) δ 8.52 (d, J=4.8 Hz, 1H), 7.66 (t, J=7.6 Hz, 1H), 7.62 (d, J=7.9 Hz, 1H), 7.57 (t, J=7.8 Hz, 1H), 7.45 (d, J=7.2 Hz, 1H), 7.33 (d, J=7.8 Hz, 1H), 7.24-7.16 (m, 1H), 6.77 (d, J=7.9 Hz, 1H), 6.68 (d, J 8.4 Hz, 1H), 5.62 (d, J=8.7 Hz, 1H), 5.39 (q, J=8.0 Hz, 1H), 3.45-3.33 (m, 1H), 2.95 (t, J=10.5 Hz, 1H), 2.56 (s, 1H), 2.00 (dd, J=12.2, 6.8 Hz, 1H), 1.91 (s, 2H), 1.62 (s, 3H), 1.61-1.57 (m, 2H), 1.56 (s, 3H), 1.54-1.46 (m, 2H). ESI-MS m/z calc. 570.10486, found 572.8 (M+3)⁺; Retention time: 1.17 minutes (LC method A).

Then, the more polar isomer eluted: (14S,17S)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (830 mg, 38%). ¹H NMR (400 MHz, Chloroform-d) δ 8.58 (s, 1H), 7.86 (d, J=8.1 Hz, 1H), 7.71 (t, J=7.7 Hz, 1H), 7.59 (s, 2H), 7.30 (d, J=7.9 Hz, 1H), 7.24 (d, J=6.7 Hz, 1H), 7.00 (s, 1H), 6.74 (s, 1H), 6.63 (s, 1H), 4.86 (s, 1H), 3.63-3.53 (m, 1H), 3.32-3.17 (m, 1H), 2.42-2.25 (m, 2H), 2.11-1.80 (m, 5H), 1.72 (t, J=12.0 Hz, 1H), 1.60 (s, 3H), 1.53 (s, 3H). ESI-MS m/z calc. 570.10486, found 572.8 (M+3)⁺; Retention time: 1.14 minutes (LC method A).

### Example 11: Preparation of (14S)-8-tert-butyl-17-(4-tert-butylpyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, Compound 76 (diastereomer 1), and Compound 75 (diastereomer 2)

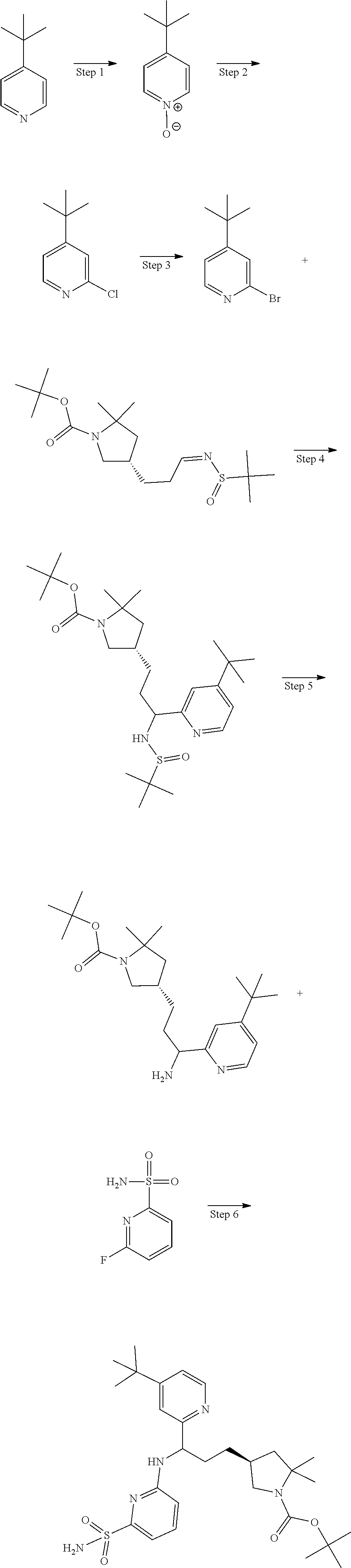

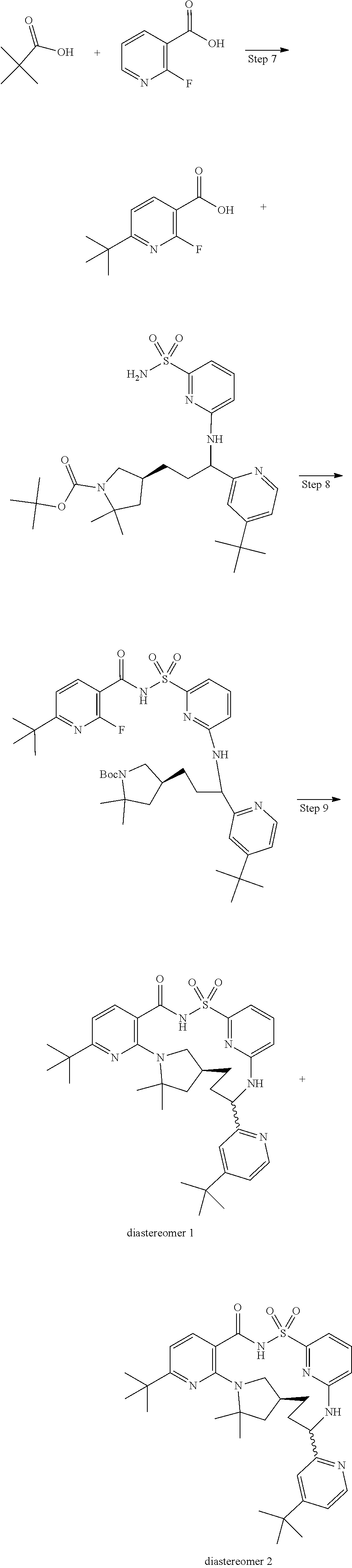

### Step 1: 4-tert-Butyl-1-oxido-pyridin-1-ium

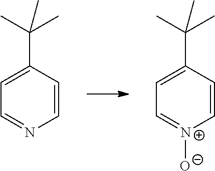

In a 2 L round bottom flask, 4-tert-butylpyridine (73.030 g, 81 mL, 529.34 mmol) was added to glacial acetic acid (600 mL). Next, hydrogen peroxide (30% in water, 450 mL) was added and the reaction mixture was refluxed under air for 4 h. Additional hydrogen peroxide (30% in water, 450 mL) was added and the reflux was continued overnight (16 h). The solvent was removed in a rotary evaporator, and the remaining solution was neutralized with a saturated sodium carbonate solution and then extracted with methylene chloride (2×500 mL) plus an chloroform/isopropanol mixture (3:1 v:v, 3×200 mL). The combined organics were dried over MgSO₄, filtered, and concentrated to give crude 4-tert-butyl-1-oxido-pyridin-1-ium (89 g, 100%). ESI-MS m/z calc. 151.0997, found 152.4 (M+1)⁺; Retention time: 1.92 minutes (LC method B).

### Step 2: 4-tert-Butyl-2-chloro-pyridine

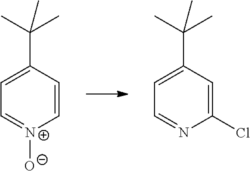

4-tert-Butyl-1-oxido-pyridin-1-ium (50.6 g, 301.18 mmol) was placed in the reaction flask and was cooled in an ice bath. POCl₃ (250 mL) was slowly added to the reaction flask to obtain a mixture. The mixture was heated under reflux for 20 h. Thereafter, the temperature was slowly reduced to 20° C., the solvent was removed from the mixture by evaporation under vacuum, a sodium carbonate aqueous solution was added for neutralization, and the contents were extracted using ethyl acetate (2×800 mL). The organic layer was washed by brine, dried over sodium sulfate and concentrated. the crude residue was purified by silica gel column chromatography using 0 to 30% ethyl acetate in hexane to afford 4-tert-butyl-2-chloro-pyridine (38.6 g, 72%) as an amber liquid. ¹H NMR (500 MHz, DMSO-d₆) δ 8.32 (dd, J=5.3, 0.7 Hz, 1H), 7.48-7.40 (m, 2H), 1.27 (s, 9H). ESI-MS m/z calc. 169.0658, found 170.3 (M+1)⁺; Retention time: 3.11 minutes (LC method B).

### Step 3: 2-Bromo-4-tert-butyl-pyridine

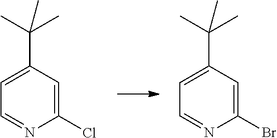

A solution of 4-tert-butyl-2-chloro-pyridine (27.07 g, 151.58 mmol) and trimethylsilyl bromide (170.52 g, 150 mL, 1.092 mol) in propionitrile (450 mL) was stirred under reflux for 21 h. The reaction flask was vacuum pumped to remove the solvents, a sodium carbonate aqueous solution was added for neutralization, and the contents in the reaction flask were extracted using ethyl acetate (2×800 mL). The organic layer was washed by brine, dried over sodium sulfate and concentrated. The crude residue was purified by silica gel column chromatography using 0% to 30% ethyl acetate in hexane to afford 2-bromo-4-tert-butyl-pyridine (34.2 g, 100%) as an amber liquid. ¹H NMR (500 MHz, DMSO-d₆) δ 8.29 (d, J=5.3 Hz, 1H), 7.58 (s, 1H), 7.46 (dd, J=5.2, 1.7 Hz, 1H), 1.27 (s, 9H). ESI-MS m/z calc. 213.0153, found 214.3 (M+1)⁺; Retention time: 5.01 minutes (LC method C).

### Step 4: tert-Butyl (4S)-4-[3-(4-tert-butyl-2-pyridyl)-3-(tert-butylsulfinylamino)propyl]-2,2-dimethyl-pyrrolidine-1-carboxylate

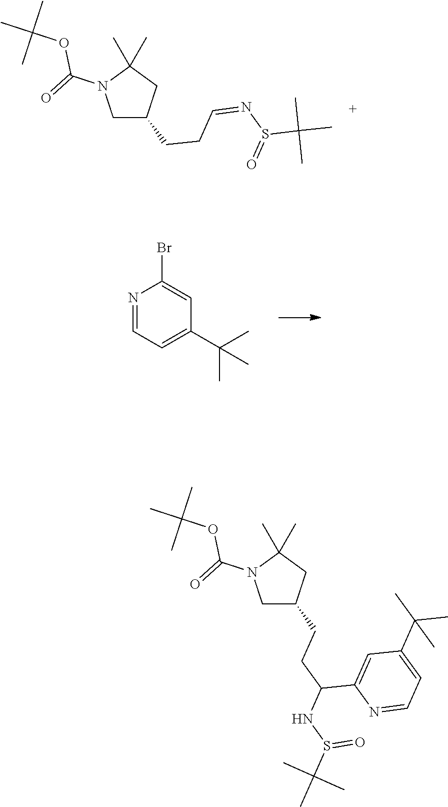

2-Bromo-4-tert-butyl-pyridine (6 g, 26.623 mmol) was dissolved in diethyl ether (60 mL) and the solution was cooled in a dry ice acetone bath (<−70° C.) under a nitrogen balloon. n-BuLi (12 mL of 2.5 M in hexanes, 30.00 mmol) was added dropwise. The mixture was stirred at this temperature for 40 min. tert-Butyl (4S)-4-[(3Z)-3-tert-butylsulfinyliminopropyl]-2,2-dimethyl-pyrrolidine-1-carboxylate (4.45 g, 11.79 mmol) was added as a THF (5 mL) solution. The mixture was stirred at −78° C. to −40° C. for 45 min. Saturated aqueous NH₄Cl (80 mL) was added. The mixture was allowed to warm to rt and partitioned between water (80 mL) and EtOAc (100 mL). This quenched mixture was separated in two layers and the aqueous layer was extracted once with EtOAc (100 mL). The combined organics was washed with brine, dried over anhydrous Na₂SO₄, filtered and concentrated. The residue was purified by silica gel column chromatography using 0 to 20% ethyl acetate in hexane to afford tert-butyl (4S)-4-[3-(4-tert-butyl-2-pyridyl)-3-(tert-butylsulfinylamino)propyl]-2,2-dimethyl-pyrrolidine-1-carboxylate (4.87 g, 79%). ESI-MS m/z calc. 493.3338, found 494.6 (M+1)⁺; Retention time: 3.01 minutes (LC method B).

### Step 5: tert-Butyl (4S)-4-[3-amino-3-(4-tert-butyl-2-pyridyl)propyl]-2,2-dimethyl-pyrrolidine-1-carboxylate

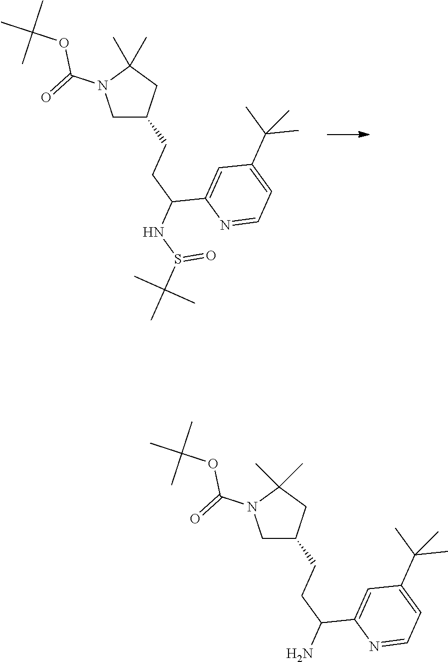

tert-Butyl (4S)-4-[3-(4-tert-butyl-2-pyridyl)-3-(tert-butylsulfinylamino)propyl]-2,2-dimethyl-pyrrolidine-1-carboxylate (4.87 g, 9.37 mmol) was dissolved in THF (43 mL) and water (8.3 mL). Molecular iodine (750 mg, 2.95 mmol) was added. The mixture was stirred at 52° C. for 3 h. It was then cooled to rt and partitioned between EtOAc (200 mL) and Na₂S₂O₃ (60 g) in saturated aqueous sodium bicarbonate (200 mL). The layers were separated and the aqueous layer was extracted once with EtOAc (100 mL). The organic layer was washed with brine, dried over anhydrous Na₂SO₄, filtered and concentrated to give tert-butyl (4S)-4-[3-amino-3-(4-tert-butyl-2-pyridyl)propyl]-2,2-dimethyl-pyrrolidine-1-carboxylate (3.9 g, 102%). ESI-MS m/z calc. 389.3042, found 390.7 (M+1)⁺; Retention time: 2.94 minutes (LC method B).

### Step 6: tert-Butyl (4S)-4-[3-(4-tert-butyl-2-pyridyl)-3-[(6-sulfamoyl-2-pyridyl)amino]propyl]-2,2-dimethyl-pyrrolidine-1-carboxylate

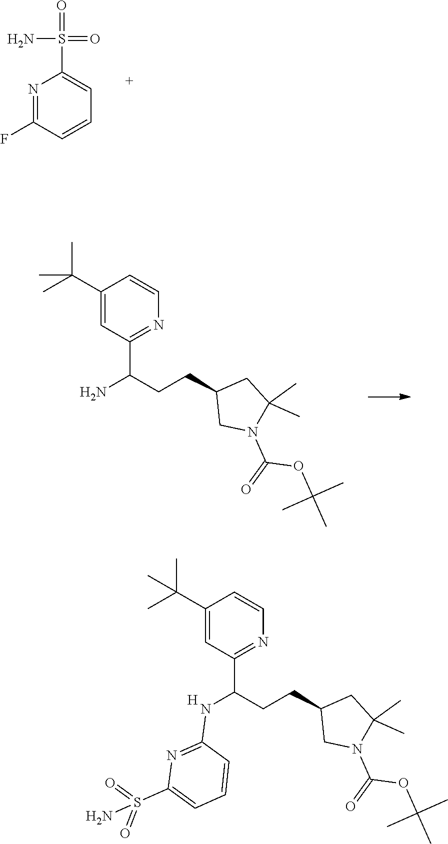

To a mixture of tert-butyl (4S)-4-[3-amino-3-(4-tert-butyl-2-pyridyl)propyl]-2,2-dimethyl-pyrrolidine-1-carboxylate (3.9 g, 9.510 mmol) and 6-fluoropyridine-2-sulfonamide (3 g, 16.178 mmol) in DMSO (10 mL) was added DIEA (5 mL, 28.706 mmol). The mixture was stirred at 115° C. for 24 h. The reaction mixture was cooled to rt. and then diluted with water (100 mL) and EtOAc (100 mL). The layers were separated and the organic layer was washed with brine (2×100 mL), dried over anhydrous Na₂SO₄, and concentrated. The residue was purified by silica gel chromatography (120 g column), using 0-80% EtOAc in hexanes to afford tert-butyl (4S)-4-[3-(4-tert-butyl-2-pyridyl)-3-[(6-sulfamoyl-2-pyridyl)amino]propyl]-2,2-dimethyl-pyrrolidine-1-carboxylate (3.66 g, 67%) as a pale color solid. ¹H NMR (500 MHz, DMSO-d₆) δ 8.43 (d, J=5.2 Hz, 1H), 7.55-7.48 (m, 2H), 7.44 (d, J=9.5 Hz, 1H), 7.24 (dd, J=5.2, 1.9 Hz, 1H), 7.07 (s, 2H), 6.95 (d, J=7.2 Hz, 1H), 6.73 (d, J=8.5 Hz, 1H), 5.22 (s, 1H), 3.57-3.43 (m, 1H), 2.81-2.65 (m, 1H), 2.10-2.01 (m, 1H), 1.94-1.73 (m, 3H), 1.48-1.35 (m, 11H), 1.35-1.29 (m, 4H), 1.26 (s, 9H), 1.21 (s, 3H). ESI-MS m/z calc. 545.3036, found 546.1 (M+1)⁺; Retention time: 2.19 minutes (LC method H).

### Step 7: 6-tert-Butyl-2-fluoro-pyridine-3-carboxylic acid

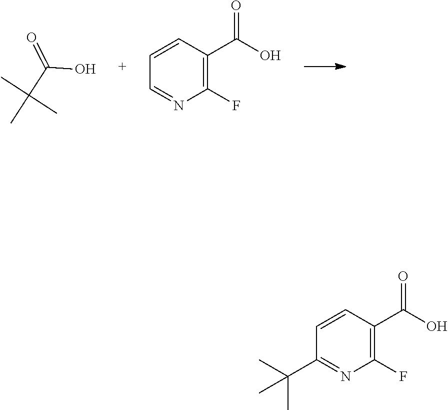

Prepared a slurry of 2-fluoropyridine-3-carboxylic acid (300 g, 2.126 mol), pivalic acid (651.5 g, 6.379 mol), and silver nitrate (54.2 g, 319.06 mmol) in water (2.4 L). Added sulfuric acid (208.5 g, 113.32 mL, 2.126 mol) dropwise over a ten minutes period: the internal temperature increased to 31° C. A solution of ammonium persulfate (970.4 g, 4.252 mol) in water (2.4 L) was then added dropwise at a rate sufficient to maintain the internal temperature between 45 and 51° C. The reaction mixture was allowed to stir overnight at room temperature, then was cooled in an ice water bath and the pH adjusted to 3-4 using aqueous NaOH (50% w/v). The mixture was diluted with DCM (2.5 L) and filtered through a pad of Celite. The solids were discarded and the phases were separated: the aqueous phase was extracted with DCM (1.7 L) and then discarded. The combined organic phases were dried over sodium sulfate and concentrated in vacuo to obtain a dark oil that was purified by silica gel chromatography using 3% methanol in dichloromethane to obtain 6-tert-butyl-2-fluoro-pyridine-3-carboxylic acid (148 g, 34%) as a pale yellow solid. ¹H NMR (500 MHz, DMSO-d₆) δ 8.33 (dd, J=10.0, 7.8 Hz, 1H), 7.49 (dd, J=7.9, 2.0 Hz, 1H), 1.30 (s, 9H). ESI-MS m/z calc. 197.0852, found 198.1 (M+1)⁺; Retention time: 1.92 minutes (LC method H).

### Step 8: tert-Butyl (4S)-4-[3-[[6-[(6-tert-butyl-2-fluoro-pyridine-3-carbonyl)sulfamoyl]-2-pyridyl]amino]-3-(4-tert-butyl-2-pyridyl)propyl]-2,2-dimethyl-pyrrolidine-1-carboxylate

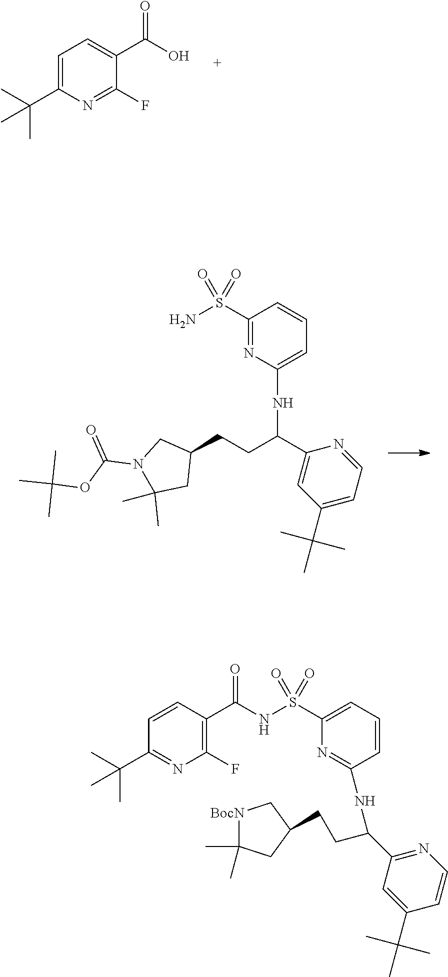 [SMILES: CC(C)(C)c1ccc(C(=O)O)c(F)n1 ; CC(C)(C)OC(=O)N1C[C@@H](CCC(Nc2cccc(S(N)(=O)=O)n2)c2cc(C(C)(C)C)ccn2)CC1(C)C ; CC(C)(C)OC(=O)N1C[C@@H](CCC(Nc2cccc(S(=O)(=O)NC(=O)c3ccc(C(C)(C)C)nc3F)n2)c2cc(C(C)(C)C)ccn2)CC1(C)C]

In a 1-L round-bottomed flask, 6-tert-butyl-2-fluoro-pyridine-3-carboxylic acid (13.8 g, 69.98 mmol) was dissolved in THF (300 mL), to which CDI (11 g, 67.84 mmol) was added. The resulting mixture was stirred at room temperature for 26 h. After this time, tert-butyl (4S)-4-[3-(4-tert-butyl-2-pyridyl)-3-[(6-sulfamoyl-2-pyridyl)amino]propyl]-2,2-dimethyl-pyrrolidine-1-carboxylate (20.0 g, 35.04 mmol) and DBU (22 mL, 147.1 mmol) were added, and the resulting mixture was stirred at room temperature for 24 h. After this time, the mixture was concentrated in vacuo. Then, the mixture was poured into ethyl acetate (1 L). This mixture was then washed with a saturated aqueous sodium bicarbonate solution (500 mL), an aqueous HCl solution (0.1 N, 500 mL) and a saturated aqueous sodium chloride solution (500 mL), then dried over sodium sulfate, filtered, and evaporated in vacuo. The resulting brown oil was purified by a silica gel plug (200 g of silica; elute with 3:1 ethyl acetate:hexanes) and was evaporated in vacuo to give a beige foam: tert-butyl (4S)-4-[3-[[6-[(6-tert-butyl-2-fluoro-pyridine-3-carbonyl)sulfamoyl]-2-pyridyl]amino]-3-(4-tert-butyl-2-pyridyl)propyl]-2,2-dimethyl-pyrrolidine-1-carboxylate (25.49 g, 100%). ESI-MS m/z calc. 724.37823, found 725.4 (M+1)⁺; Retention time: 1.89 minutes (LC method A).

### Step 9: (14S)-8-tert-Butyl-17-(4-tert-butylpyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, Compound 76 (diastereomer 1) and (14S)-8-tert-butyl-17-(4-tert-butylpyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, Compound 75 (diastereomer 2)

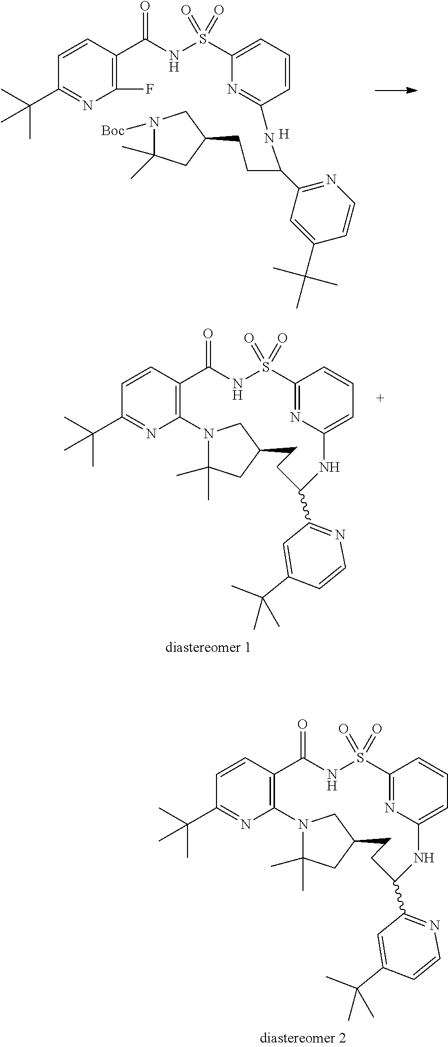

Stage 1: In a 1-L round-bottomed flask, tert-butyl (4S)-4-[3-[[6-[(6-tert-butyl-2-fluoro-pyridine-3-carbonyl)sulfamoyl]-2-pyridyl]amino]-3-(4-tert-butyl-2-pyridyl)propyl]-2,2-dimethyl-pyrrolidine-1-carboxylate (25.49 g, 35.16 mmol), was dissolved in dichloromethane (400 mL). TFA (50 mL, 649.0 mmol) was added, and the resulting solution was allowed to stand at room temperature for 4 h. The mixture was then evaporated in vacuo, diluted with dioxane, and evaporated in vacuo again. This gave an orange oil, ˜45 g (>100% yield).

Stage 2: In a 1-L round-bottomed flask, the crude product from Step 1 was dissolved in NMP (400 mL), to which K₂CO₃ (40.72 g, 294.6 mmol) was added. The resulting mixture was flushed with nitrogen, then stirred at 150° C. for 17 h. After cooling to room temperature, the reaction mixture was concentrated in vacuo to ˜¼ of the original volume. The resulting mixture was poured into a 2-L flask containing cold water (400 mL). [Note: effervescence.] This was then mixed with aqueous HCl solution (1 N; 700 mL), then extracted with ethyl acetate (2×800 mL). The combined organic extracts was washed with water (1 L) and saturated aqueous sodium chloride solution (1 L), then dried over sodium sulfate, filtered, and evaporated in vacuo. A brown gum (˜30 g) was obtained as the crude product. Several purifications by silica gel chromatography gave two separate products, which were further purified by precipitation from ethanol/water.

Diastereomer 1: “Peak 1”, (14S)-8-tert-butyl-17-(4-tert-butylpyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (3.6701 g, 17%); ¹H NMR (400 MHz, DMSO-d₆) δ 12.23 (s, 1H), 8.44 (d, J=5.2 Hz, 1H), 7.68 (d, J=8.1 Hz, 1H), 7.62 (t, J 7.9 Hz, 1H), 7.55 (s, 1H), 7.49 (d, J=7.5 Hz, 1H), 7.25 (d, J=4.4 Hz, 1H), 7.21 (d, J 7.1 Hz, 1H), 6.86 (d, J=8.5 Hz, 1H), 6.63 (d, J=7.9 Hz, 1H), 5.11-4.77 (m, 1H), 3.22-3.00 (m, 2H), 2.37-2.24 (m, 1H), 2.18-2.07 (m, 1H), 1.85 (dd, J=11.9, 5.9 Hz, 1H), 1.75-1.65 (m, 1H), 1.60 (s, 3H), 1.52 (s, 3H), 1.51-1.38 (m, 1H), 1.27 (s, 9H), 1.23 (s, 9H), 1.20-1.07 (m, 1H). [Note: 1H is missing from the overall count of 44 from the product (C33H44N6O3S)]. ESI-MS m/z calc. 604.3196, found 605.4 (M+1)⁺; Retention time: 1.72 minutes (LC method A).

Diastereomer 2: “Peak 2, (14S)-8-tert-butyl-17-(4-tert-butylpyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (4.7440 g, 22%); ¹H NMR (400 MHz, DMSO-d₆) δ 12.41 (s, 1H), 8.40 (d, J=5.2 Hz, 1H), 7.69-7.57 (m, 3H), 7.51-7.43 (m, 1H), 7.23 (dd, J=5.3, 1.9 Hz, 1H), 7.09 (d, J=7.2 Hz, 1H), 6.87 (d, J=8.4 Hz, 1H), 6.66 (d, J 8.0 Hz, 1H), 5.32-5.22 (m, 1H), 3.28-3.19 (m, 1H), 2.76 (t, J 10.5 Hz, 1H), 2.28-2.15 (m, 1H), 1.96-1.87 (m, 2H), 1.84 (dd, J=11.8, 5.3 Hz, 1H), 1.77-1.67 (m, 1H), 1.65 (s, 3H), 1.57 (t, J=12.4 Hz, 1H), 1.51 (s, 3H), 1.49-1.41 (m, 1H), 1.28 (s, 9H), 1.23 (s, 9H). ESI-MS m/z calc. 604.3196, found 605.3 (M+1)⁺; Retention time: 1.76 minutes (LC method A).

### Example 12: Preparation of (14S)-8-tert-butyl-12,12-dimethyl-17-(pyridin-2-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 293 (diastereomer 1), and Compound 292 (diastereomer 2)

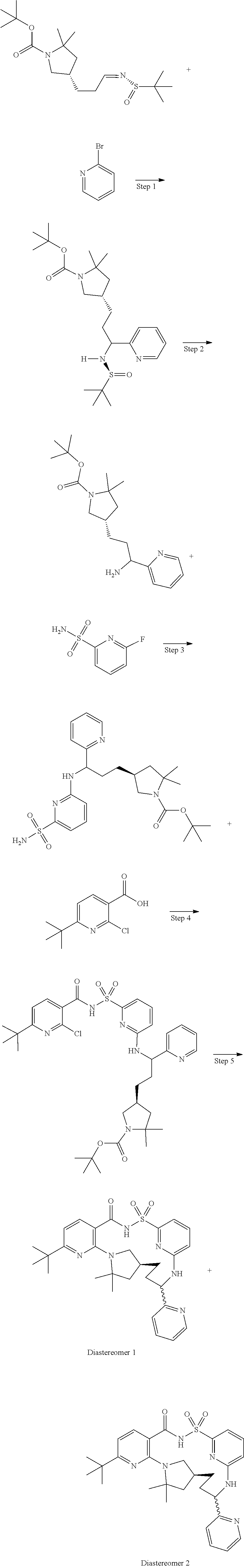

### Step 1: tert-Butyl (4S)-4-[3-[[(S)-tert-butylsulfinyl]amino]-3-(2-pyridyl)propyl]-2,2-dimethyl-pyrrolidine-1-carboxylate

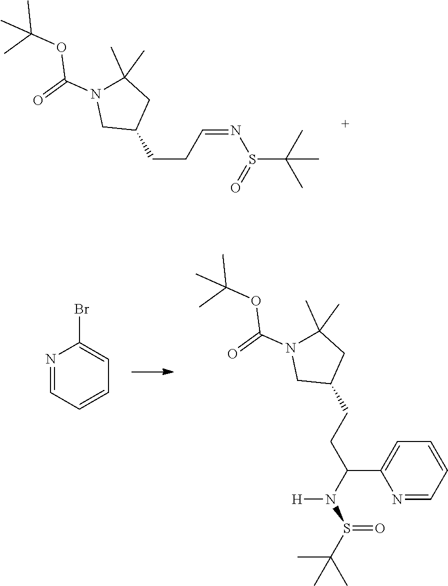

2-Bromopyridine (0.8 mL, 8.223 mmol) was dissolved in THF (24 mL) and the solution was cooled in a dry ice acetone bath (<−70° C.) under a nitrogen balloon. n-BuLi (3 mL of 2.5 M in hexanes, 7.500 mmol) was added. The reddish colored mixture was stirred at this temperature for 15 min. tert-butyl (4S)-4[(3Z)-3-[(S)-tert-butylsulfinyl]iminopropyl]-2,2-dimethyl-pyrrolidine-1-carboxylate; methane (1.5 g, 3.9745 mmol) was added as a THF (4 mL) solution. The mixture was stirred at −78° C. to −40° C. for 30 min. Saturated aqueous NH₄Cl (20 mL) was added. The mixture was allowed to warm up to rt and partitioned between water (80 mL) and EtOAc (100 mL). The layers were separated and the aqueous layer was extracted with more EtOAc (100 mL). The combined organic layer was washed with brine, dried over sodium sulfate concentrated to afford tert-butyl (4S)-4-[3-[[(S)-tert-butylsulfinyl]amino]-3-(2-pyridyl)propyl]-2,2-dimethyl-pyrrolidine-1-carboxylate (2.5 g, 101%). ESI-MS m/z calc. 437.2712, found 438.6 (M+1)⁺; Retention time: 2.67 minutes (LC method B).

### Step 2: tert-Butyl (4S)-4-[3-amino-3-(2-pyridyl)propyl]-2,2-dimethyl-pyrrolidine-1-carboxylate

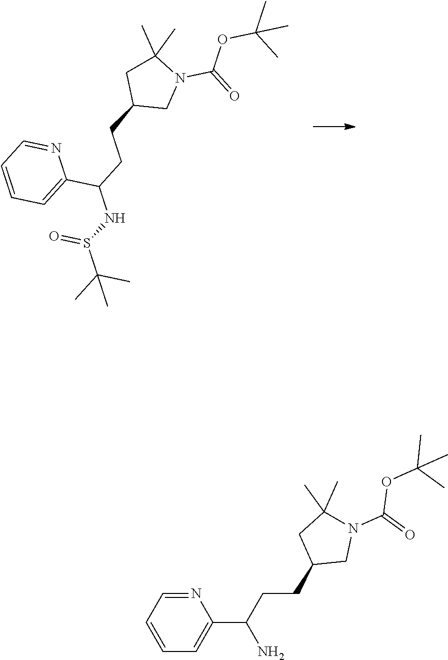 [SMILES: CC(C)(C)OC(=O)N1C[C@@H](CCC(N[S@@](=O)C(C)(C)C)c2ccccn2)CC1(C)C ; CC(C)(C)OC(=O)N1C[C@@H](CCC(N)c2ccccn2)CC1(C)C]

tert-Butyl (4S)-4-[3-[[(S)-tert-butylsulfinyl]amino]-3-(2-pyridyl)propyl]-2,2-dimethyl-pyrrolidine-1-carboxylate (13.5 g, 26.220 mmol) was dissolved in THF (180 mL) and water (36 mL). Molecular iodine (2 g, 7.880 mmol) was added. The mixture was stirred at 35° C. for 16 h. It was then cooled to rt and partitioned between EtOAc (300 mL) and Na₂S₂O₃ (50 g) in saturated aqueous sodium bicarbonate (300 mL). The layers were separated and the aqueous layer was extracted once with EtOAc (200 mL). The organic layer was concentrated. The residue was dissolved in 1M HCl (500 mL) and was extracted with diethyl ether (300 mL). The aqueous layer was basified by 2.5M NaOH and extracted with EtOAc (2×300 mL). The organic layer was washed with brine, dried over anhydrous Na₂SO₄, filtered and concentrated to give tert-butyl (4S)-4-[3-amino-3-(2-pyridyl)propyl]-2,2-dimethyl-pyrrolidine-1-carboxylate (8.5 g, 92%). ESI-MS m/z calc. 333.2416, found 334.6 (M+1)⁺; Retention time: 2.55 minutes (LC method B).

### Step 3: tert-Butyl (4S)-2,2-dimethyl-4-[3-(2-pyridyl)-3-[(6-sulfamoyl-2-pyridyl)amino]propyl]pyrrolidine-1-carboxylate

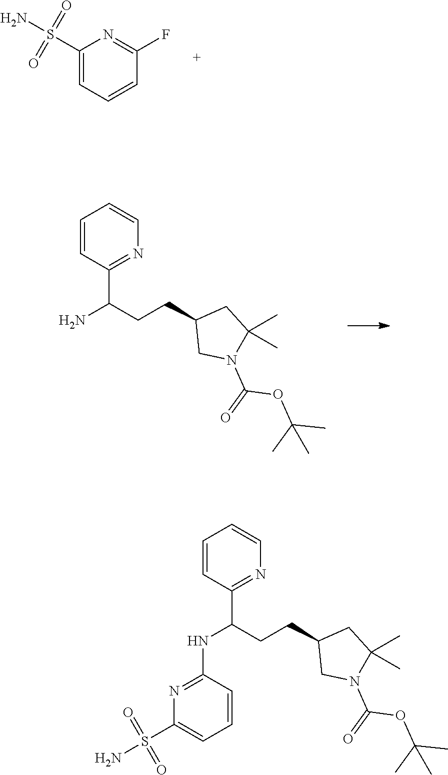 [SMILES: NS(=O)(=O)c1cccc(F)n1 ; CC(C)(C)OC(=O)N1C[C@@H](CCC(N)c2ccccn2)CC1(C)C ; CC(C)(C)OC(=O)N1C[C@@H](CCC(Nc2cccc(S(N)(=O)=O)n2)c2ccccn2)CC1(C)C]

To a mixture of tert-butyl (4S)-4-[3-amino-3-(2-pyridyl)propyl]-2,2-dimethyl-pyrrolidine-1-carboxylate (8.5 g, 24.215 mmol) and 6-fluoropyridine-2-sulfonamide (8 g, 43.140 mmol) in DMSO (22 mL) was added DIEA (12 mL, 68.893 mmol). The mixture was stirred at 115° C. for 20 h. It was then cooled to rt and partitioned between EtOAc (300 mL) and saturated sodium bicarbonate (300 mL). The aqueous layer was extracted with more EtOAc (300 mL). The combined EtOAc solution was washed with brine (2×500 mL), dried over anhydrous Na₂SO₄, filtered and concentrated. The residue was purified by silica gel chromatography, using 5-100% EtOAc in hexanes to afford tert-butyl (4S)-2,2-dimethyl-4-[3-(2-pyridyl)-3-[(6-sulfamoyl-2-pyridyl)amino]propyl]pyrrolidine-1-carboxylate (10.41 g, 83%) as a solid. ¹H NMR (500 MHz, DMSO-d₆) δ 8.54 (d, J=4.6 Hz, 1H), 7.72 (td, J=7.7, 7.6, 1.8 Hz, 1H), 7.57-7.42 (m, 3H), 7.28-7.20 (m, 1H), 7.06 (s, 2H), 6.96 (d, J=7.2 Hz, 1H), 6.76-6.68 (m, 1H), 5.19 (s, 1H), 3.58-3.48 (m, 1H), 2.83-2.69 (m, 1H), 2.14-2.02 (m, 1H), 1.95-1.78 (m, 3H), 1.42-1.30 (m, 15H), 1.22 (s, 3H).ESI-MS m/z calc. 489.241, found 490.3 (M+1)⁺; Retention time: 1.82 minutes (LC method H).

### Step 4: tert-Butyl (4S)-4-[3-[[6-[(6-tert-butyl-2-chloro-pyridine-3-carbonyl)sulfamoyl]-2-pyridyl]amino]-3-(2-pyridyl)propyl]-2,2-dimethyl-pyrrolidine-1-carboxylate

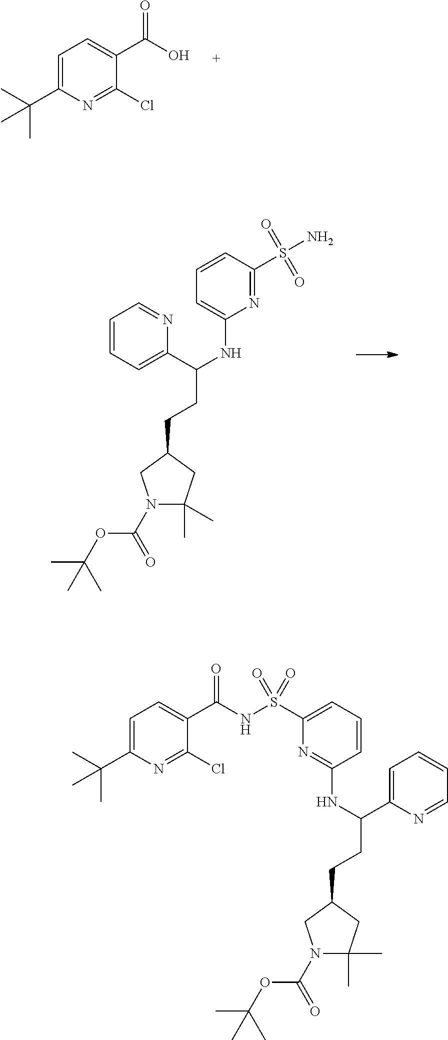 [SMILES: CC(C)(C)c1ccc(C(=O)O)c(Cl)n1 ; CC(C)(C)OC(=O)N1C[C@@H](CCC(Nc2cccc(S(N)(=O)=O)n2)c2ccccn2)CC1(C)C ; CC(C)(C)OC(=O)N1C[C@@H](CCC(Nc2cccc(S(=O)(=O)NC(=O)c3ccc(C(C)(C)C)nc3Cl)n2)c2ccccn2)CC1(C)C]

To a solution of 6-tert-butyl-2-chloro-pyridine-3-carboxylic acid (400 mg, 1.872 mmol) in THF (10 mL) was added CDI (310 mg, 1.912 mmol) (recrystallized from THF) and the mixture was stirred at rt for 3 h then tert-butyl (4S)-2,2-dimethyl-4-[3-(2-pyridyl)-3-[(6-sulfamoyl-2-pyridyl)amino]propyl]pyrrolidine-1-carboxylate (555 mg, 1.134 mmol) was added followed by DBU (550 μL, 3.678 mmol) and the resulting mixture was stirred for 16 h at rt. The reaction was diluted with ethyl acetate and washed with a saturated aqueous sodium bicarbonate solution. The organic layer was further washed with a 10% citric acid solution followed by brine. The organics were separated, dried over sodium sulfate, evaporated and then purified on silica gel chromatography (80 gram column) using a gradient from 100% hexanes to 100% ethyl acetate to afford tert-butyl (4S)-4-[3-[[6-[(6-tert-butyl-2-chloro-pyridine-3-carbonyl)sulfamoyl]-2-pyridyl]amino]-3-(2-pyridyl)propyl]-2,2-dimethyl-pyrrolidine-1-carboxylate as an off-white solid (450 mg, 58%). ESI-MS m/z calc. 684.2861, found 685.2 (M+1)⁺; Retention time: 1.76 minutes (LC method A).

### Step 5: (14S)-8-tert-Butyl-12,12-dimethyl-17-(pyridin-2-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 293 (diastereomer 1), and (14S)-8-tert-butyl-12,12-dimethyl-17-(pyridin-2-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 292 (diastereomer 2)

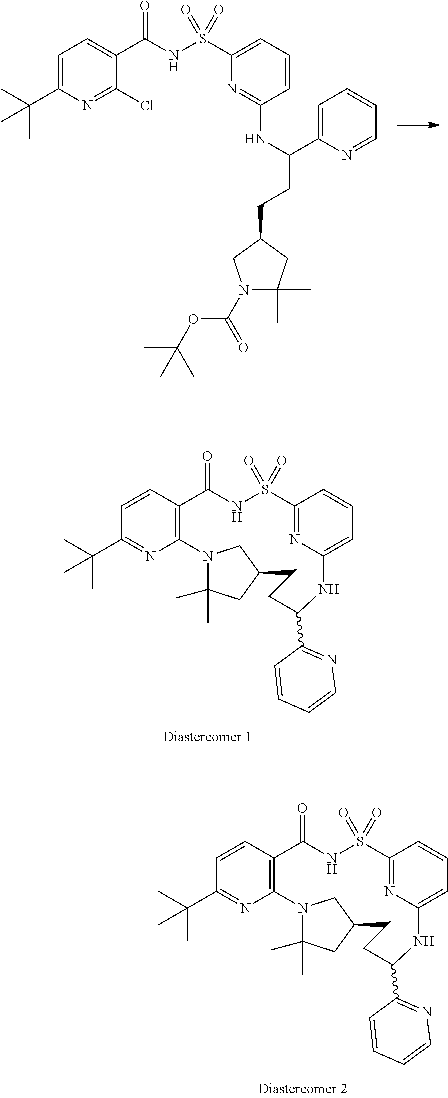 [SMILES: CC(C)(C)OC(=O)N1C[C@@H](CCC(Nc2cccc(S(=O)(=O)NC(=O)c3ccc(C(C)(C)C)nc3Cl)n2)c2ccccn2)CC1(C)C ; CC(C)(C)c1ccc2c(n1)N1C[C@@H](CCC(c3ccccn3)Nc3cccc(n3)S(=O)(=O)NC2=O)CC1(C)C ; CC(C)(C)c1ccc2c(n1)N1C[C@@H](CCC(c3ccccn3)Nc3cccc(n3)S(=O)(=O)NC2=O)CC1(C)C]

Stage 1: tert-Butyl (4S)-4-[3-[[6-[(6-tert-butyl-2-chloro-pyridine-3-carbonyl)sulfamoyl]-2-pyridyl]amino]-3-(2-pyridyl)propyl]-2,2-dimethyl-pyrrolidine-1-carboxylate (450.2 mg, 0.6569 mmol) was dissolved in DCM (20 mL) and to the mixture was added HCl (5 mL of 4 M in dioxane, 20.00 mmol) and the reaction was stirred at room temperature. After 2 h, the reaction was complete. The reaction was basified with aqueous sodium carbonate until pH˜8. Then diluted with ethyl acetate and washed with a saturated aqueous sodium bicarbonate solution. The organic layer was extracted and then further washed with brine. The organics were separated, dried over sodium sulfate, evaporated and then placed on the high vacuum pump for 1 h to afford the intermediate 6-tert-butyl-2-chloro-N-[[6-[[3-[(3S)-5,5-dimethylpyrrolidin-3-yl]-1-(2-pyridyl)propyl]amino]-2-pyridyl]sulfonyl]pyridine-3-carboxamide as an off-white solid (hydrochloride salt). ESI-MS m/z calc. 584.23364, found 585.2 (M+1)⁺; Retention time: 1.07 minutes (LC method A).

Stage 2:Combined material from stage 1 and K₂CO₃ (1 g, 7.236 mmol), 3 Å molecular sieves and DMSO (20 mL) in a vial, purged with nitrogen, capped, heated to 155° C. and stirred for 72 h. The mixture was cooled to ambient temperature, filtered and concentrated under a stream of nitrogen to give a residue which was purified by reverse-phase preparative chromatography utilizing a C₁₈ column and an HPLC-MS method using a 25-60% gradient of acetonitrile in water (+5 mM HCl), 30 minute) to afford two product peaks, separated diastereomers:

Diastereomer 1, more polar, off-white solid: (14S)-8-tert-Butyl-12,12-dimethyl-17-(pyridin-2-yl)-2λ⁶-thia-3,9,11,18,23-pentaazatetracyclo[17.3.1.111,14.05,10]tetracosa-1(23),5(10),6,8,19,21-hexaene-2,2,4-tri one (hydrochloride salt) (3.8 mg, 3%). ESI-MS m/z calc. 548.25696, found 549.2 (M+1)⁺; Retention time: 1.55 minutes (LC method A).

Diastereomer 2, less polar, off-white solid: (14S)-8-tert-Butyl-12,12-dimethyl-17-(pyridin-2-yl)-2λ⁶-thia-3,9,11,18,23-pentaazatetracyclo[17.3.1.111,14.05,10]tetracosa-1(23),5(10),6,8,19,21-hexaene-2,2,4-tri one (hydrochloride salt) (36.85 mg, 27%). ¹H NMR (500 MHz, DMSO-d₆) δ 12.50 (s, 1H), 8.85 (d, J=5.8 Hz, 1H), 8.57 (t, J=7.9 Hz, 1H), 8.24 (d, J=8.5 Hz, 1H), 8.16 (d, J=8.2 Hz, 1H), 7.94 (t, J=6.7 Hz, 1H), 7.74 (t, J=7.9 Hz, 1H), 7.63 (d, J=8.0 Hz, 1H), 7.21 (d, J=7.2 Hz, 1H), 7.01 (d, J=8.4 Hz, 1H), 6.65 (d, J=7.9 Hz, 1H), 5.57 (d, J=7.5 Hz, 1H), 3.39 (t, J=8.5 Hz, 1H), 2.66 (t, J=10.1 Hz, 1H), 2.50-2.38 (m, 2H), 2.07 (s, 2H), 1.89-1.73 (m, 2H), 1.65 (s, 3H), 1.55 (t, J=12.3 Hz, 1H), 1.50 (s, 3H), 1.27 (s, 9H). ESI-MS m/z calc. 548.25696, found 549.2 (M+1)⁺; Retention time: 1.63 minutes (LC method A).

The following is a list of alkyl, aryl and heteroaryl halide reagents that are commercially available:
3-Bromopyridine
3-Bromo-1-methyl-pyrazole
4-Iodopyridine
5-Bromopyrimidine
2-Bromopyrimidine
2-Bromo-6-(trifluoromethyl)pyridine
2-Bromo-4-(trifluoromethyl)pyridine
1-Bromo-3-tert-buty 1-benzene
1-Bromo-4-tert-buty 1-benzene
2-Bromo-5-(trifluoromethyl)pyridine
2-Bromo-3-chloro-pyridine

The compounds in the following tables were prepared in a manner analogous to that described above using commercially available alkyl, aryl and heteroaryl halide reagents given in the table above.

For the preparation of Compound 279 and Compound 278, the lithiating agent in step 1 was t-BuLi. For the preparation of Compound 217 and Compound 216, the lithiating agent in step 1 was LDA. For the preparation of Compound 285 and Compound 284, the lithiated reagent in step 1 was generated by reacting n-BuLi with 1-methylpyrazole at −70° C.

For the preparation of Compound 245 and Compound 244, the lithiated reagent in step 1 was commercially available t-BuLi.

Unless otherwise stated, for each prepared diastereomeric pair, the diastereomer 1 was the first isomer to elute during the separation procedure. The diastereomer 2 was the second isomer to elute.

The diastereomers in the following table were separated by the following methods:

| Compound | |
|---|---|
| Number | Separation Method |
| Compound 283 | Preparative HPLC, C₁₈ column and |
| (diastereomer | HPLC-MS method 10-60% gradient of acetonitrile |
| 1) and | in water + 5 mM HCl, over 15 minutes |
| Compound 282 | |
| (diastereomer 2) | |
| Compound 279 | Preparative SFC using Phenomenex LUX-4 (250 × |
| (diastereomer | 21.2 mm, 5 μm) column, 35° C., mobile phase |
| 1) and | 34% MeOH (no modifier), 66% CO₂, flow |
| Compound 278 | 10 mL/min, concentrations 28 mg/mL in MeOH |
| (diastereomer 2) | (no modifier), injection volume 70 μL, pressure |
| | 138 bar, wavelength 210 nm |
| Compound 233 | Preparative HPLC C₁₈ column and |
| (diastereomer | HPLC-MS method 1-60% gradient of acetonitrile |
| 1) and | in water + 5 mM HCl, over 15 minutes |
| Compound 232 | |
| (diastereomer 2) | |
| Compound 217 | Preparative HPLC C₁₈ column and |
| (diastereomer | HPLC-MS method 30-99% gradient of |
| 1) and | acetonitrile-water + 5 mM HCl, over 15 minutes |
| Compound 216 | |
| (diastereomer 2) | |
| Compound 207 | Preparative SFC chromatography using a Phenomenex |
| (diastereomer | LUX-4 column (250 × 21.2 mm column, 5 μm |
| 1) and | particle size) and a dual gradient run 50 to 80% |
| Compound 206 | mobile phase B over 14.5 min. (mobilie phase |
| (diastereomer 2) | A = CO₂, mobile phase B = MeOH |
| | (containing 20 mM NH₃), flow rate = |
| | 40 mL/min and column temperature = |
| | 40° C. |
| Compound 95 | Preparative SFC eluting a gradient of 5 mM NH₃ |
| (diastereomer | in methanol to CO₂ (40-70% over 10 min) |
| 1) and | through a 21.2 × 250 mm 2-PIC column, 5 μm |
| Compound 94 | particle |
| (diastereomer 2) | |
| Compound 93 | Preparative SFC eluting a gradient of 5 mM NH₃ |
| (diastereomer | in methanol to CO₂ (40-70% over 10 min) |
| 1) and | through a 21.2 × 250 mm 2-PIC column, 5 μm |
| Compound 92 | particle |
| (diastereomer 2) | |
| Compound 81 | Preparative SFC eluting a gradient of 5 mM NH₃ |
| (diastereomer | in methanol to CO₂ (40-70% over 10 min) |
| 2) and | through a 21.2 × 250 mm 2-PIC column, 5 μm |
| Compound 80 | particle. |
| (diastereomer 1) | |
| Compound 41 | Silica gel chromatography (40 g of silica) using a |
| (diastereomer | gradient eluent of 1 to 50% EtOAc in hexanes. |
| 1) and | Diastereomer 2 (less polar) eluted first and |
| Compound 40 | diastereomer 1 (more polar) eluted last. |
| (diastereomer 2) | |
| Compound 38 | Silica gel chromatography (24 g silica) using a |
| (diastereomer | gradient eluent of 0 to 40% EtOAc in hexanes. |
| 1) and | Diastereomer 2(less polar) eluted first and |
| Compound 37 | diastereomer 1 (more polar) eluted last. |
| (diastereomer 2) | |
| Compound 66 | Silica gel chromatography (220 g silica) using a |
| (diastereomer | gradient from 100% hexanes to 100% ethyl acetate. |
| 1) and | Diastereomer 1 (less polar) eluted first and |
| Compound 65 | Diastereomer 2 (more polar) eluted last. |
| (diastereomer 2) | |
| Compound 285 | Preparative SFC chromatography using a Regis-[R,R]- |
| (Diastereomer | Whelk-O (250 × 10 mm column, 5 μm particle size) |
| 1) and | with 28% MeOH/72% CO₂ mobile phase at |
| Compound 284 | 10 mL/min over 6.0 minutes (injection volume = |
| (Diastereomer 2) | 70 μL of 23 mg/mL solution in 88/12 MeOH/DMSO |
| Compound 245 | Preparative HPLC C₁₈ column and HPLC-MS method |
| (Diastereomer | 30-99% gradient of acetonitrile-water + 5 mM HCl, |
| 1) and | over 15 minutes |
| Compound 244 | |
| (Diastereomer 2) | |

| | | LCMS | | | |
|---|---|---|---|---|---|
| | | Retention | | | |
| Compound | | Time | Exact | | LCMS |
| Number | Structure | (min) | Mass | M + 1 | Method |
| Compound 283 (diastereomer 1), hydrochloride salt | 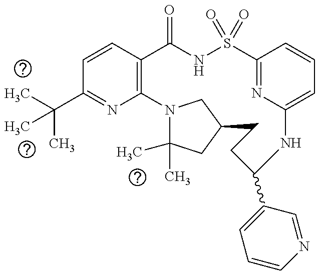 [SMILES: CC(C)(C)c1ccc2c(n1)N1C[C@@H](CCC(c3cccnc3)Nc3cccc(n3)S(=O)(=O)NC2=O)CC1(C)C] | 1.53 | 548.257 | 549.2 | LC method A |
| Compound 282 (diastereomer 2), hydrochloride salt | 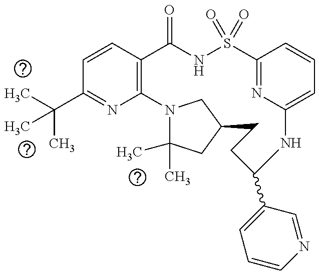 [SMILES: CC(C)(C)c1ccc2c(n1)N1C[C@@H](CCC(c3cccnc3)Nc3cccc(n3)S(=O)(=O)NC2=O)CC1(C)C] | 1.62 | 548.257 | 549.2 | LC method A |
| Compound 279 (diastereomer 1) | 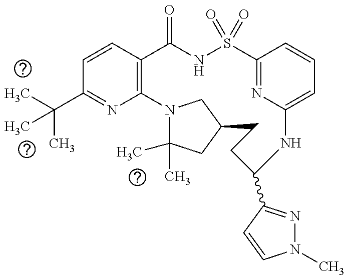 [SMILES: Cn1ccc(C2CC[C@@H]3CN(c4nc(C(C)(C)C)ccc4C(=O)NS(=O)(=O)c4cccc(n4)N2)C(C)(C)C3)n1] | 1.97 | 551.268 | 552.2 | LC method A |
| Compound 278 (diastereomer 2) | 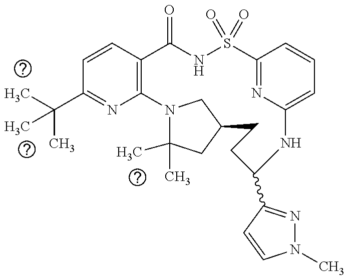 [SMILES: Cn1ccc(C2CC[C@@H]3CN(c4nc(C(C)(C)C)ccc4C(=O)NS(=O)(=O)c4cccc(n4)N2)C(C)(C)C3)n1] | 1.92 | 551.268 | 552.2 | LC method A |
| Compound 233 (diastereomer 1), hydrochloride salt | 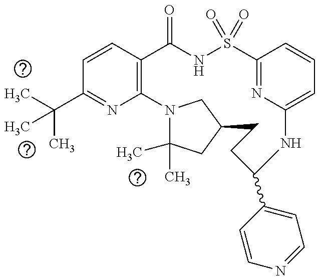 [SMILES: CC(C)(C)c1ccc2c(n1)N1C[C@@H](CCC(c3ccncc3)Nc3cccc(n3)S(=O)(=O)NC2=O)CC1(C)C] | 1.5 | 548.257 | 549.2 | LC method A |
| Compound 232 (diastereomer 2), hydrochloride salt | 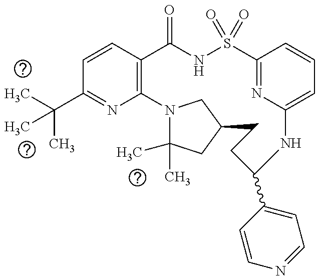 [SMILES: CC(C)(C)c1ccc2c(n1)N1C[C@@H](CCC(c3ccncc3)Nc3cccc(n3)S(=O)(=O)NC2=O)CC1(C)C] | 1.61 | 548.257 | 549.2 | LC method A |
| Compound 217 (diastereomer 1) | 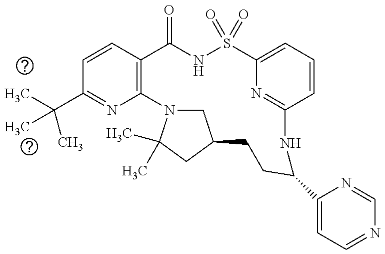 [SMILES: CC(C)(C)c1ccc2c(n1)N1C[C@@H](CC[C@@H](c3ccncn3)Nc3cccc(n3)S(=O)(=O)NC2=O)CC1(C)C] | 1.32 | 549.252 | 550.3 | LC method G |
| Compound 216 (diastereomer 2) | 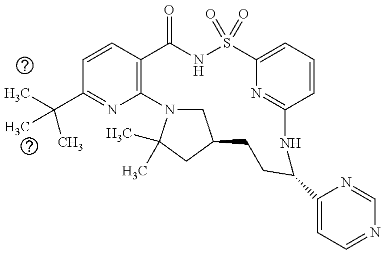 [SMILES: CC(C)(C)c1ccc2c(n1)N1C[C@@H](CC[C@@H](c3ccncn3)Nc3cccc(n3)S(=O)(=O)NC2=O)CC1(C)C] | 1.43 | 549.252 | 550.4 | LC method G |
| Compound 207 (diastereomer 1) | 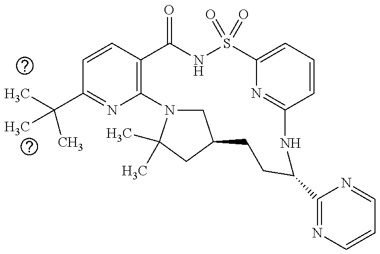 [SMILES: CC(C)(C)c1ccc2c(n1)N1C[C@@H](CC[C@@H](c3ncccn3)Nc3cccc(n3)S(=O)(=O)NC2=O)CC1(C)C] | 3 | 549.252 | 550.3 | LC method A |
| Compound 206 (diastereomer 2) | 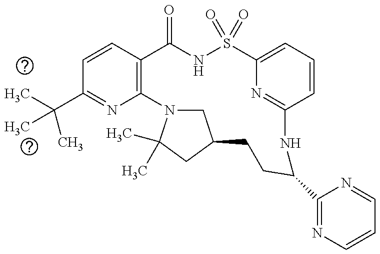 [SMILES: CC(C)(C)c1ccc2c(n1)N1C[C@@H](CC[C@@H](c3ncccn3)Nc3cccc(n3)S(=O)(=O)NC2=O)CC1(C)C] | 2.99 | 549.252 | 550.4 | LC method A |
| Compound 95 (diastereomer 1) | 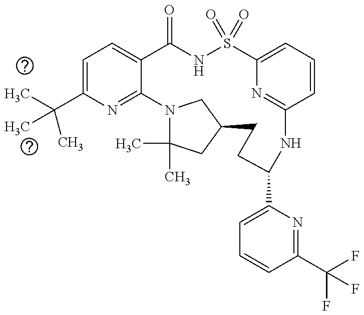 [SMILES: CC(C)(C)c1ccc2c(n1)N1C[C@@H](CC[C@@H](c3cccc(C(F)(F)F)n3)Nc3cccc(n3)S(=O)(=O)NC2=O)CC1(C)C] | 1.9 | 616.244 | 617.2 | LC method A |
| Compound 94 (diastereomer 2) | 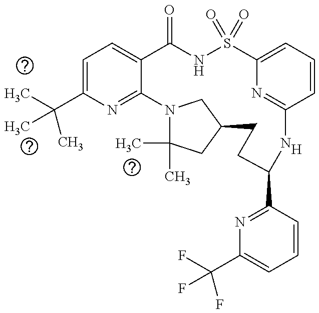 | 1.89 | 616.244 | 617.1 | LC method A |
| Compound 93 (diastereomer 1) | 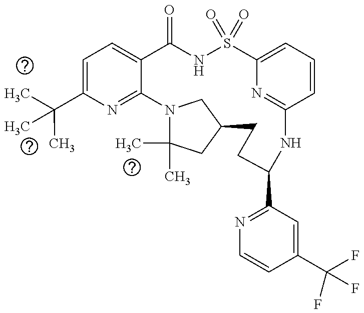 [SMILES: CC(C)(C)c1ccc2c(n1)N1C[C@@H](CC[C@H](c3cc(C(F)(F)F)ccn3)Nc3cccc(n3)S(=O)(=O)NC2=O)CC1(C)C] | 1.83 | 616.244 | 617.2 | LC method A |
| Compound 92 (diastereomer 2) | 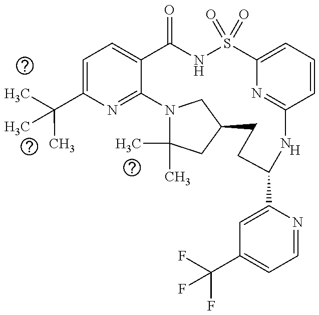 | 1.83 | 616.244 | 617.2 | LC method A |
| Compound 41 (diastereomer 1) | 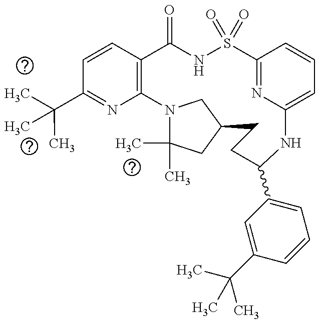 [SMILES: CC(C)(C)c1cccc(C2CC[C@@H]3CN(c4nc(C(C)(C)C)ccc4C(=O)NS(=O)(=O)c4cccc(n4)N2)C(C)(C)C3)c1] | 2.42 | 603.324 | 604.3 | LC method A |
| Compound 40 (diasteromer 2) | 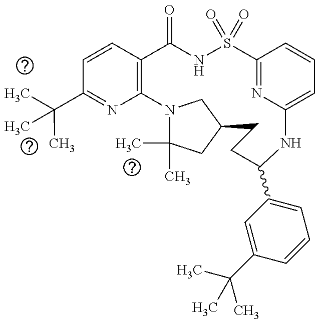 [SMILES: CC(C)(C)c1cccc(C2CC[C@@H]3CN(c4nc(C(C)(C)C)ccc4C(=O)NS(=O)(=O)c4cccc(n4)N2)C(C)(C)C3)c1] | 2.5 | 603.324 | 604.4 | LC method A |
| Compound 38 (diastereomer 1) | 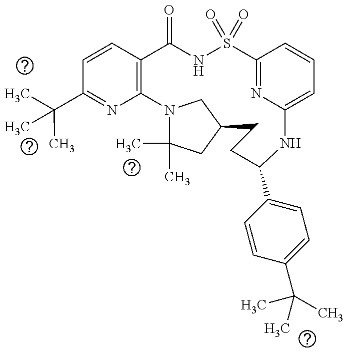 [SMILES: CC(C)(C)c1ccc([C@@H]2CC[C@@H]3CN(c4nc(C(C)(C)C)ccc4C(=O)NS(=O)(=O)c4cccc(n4)N2)C(C)(C)C3)cc1] | 2.43 | 603.324 | 604.3 | LC method A |
| Compound 37 (diastereomer 2) | 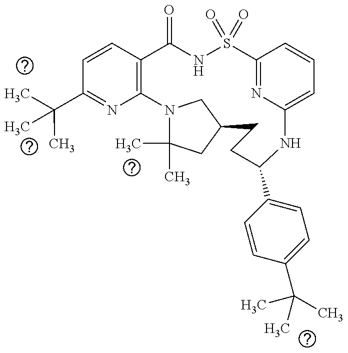 [SMILES: CC(C)(C)c1ccc([C@@H]2CC[C@@H]3CN(c4nc(C(C)(C)C)ccc4C(=O)NS(=O)(=O)c4cccc(n4)N2)C(C)(C)C3)cc1] | 2.51 | 603.324 | 604.4 | LC method A |
| Compound 81 (diastereomer 2) | 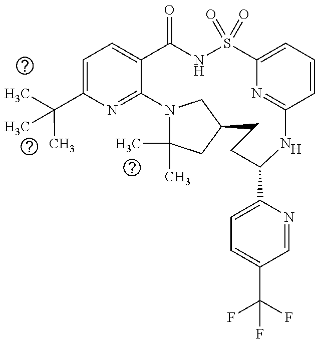 | 1.88 | 616.244 | 617.1 | LC method A |
| Compound 80 (diastereomer 1) | 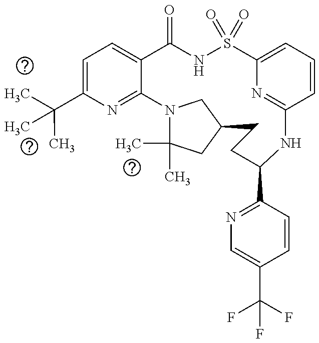 [SMILES: CC(C)(C)c1ccc2c(n1)N1C[C@@H](CC[C@H](c3ccc(C(F)(F)F)cn3)Nc3cccc(n3)S(=O)(=O)NC2=O)CC1(C)C] | 1.87 | 616.244 | 617.1 | LC method A |
| Compound 66 (diastereomer 1) | 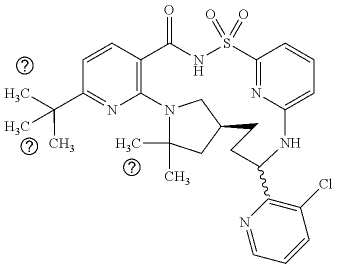 [SMILES: CC(C)(C)c1ccc2c(n1)N1C[C@@H](CCC(c3ncccc3Cl)Nc3cccc(n3)S(=O)(=O)NC2=O)CC1(C)C] | 2.11 | 582.218 | 583.2 | LC method A |
| Compound 65 (diastereomer 2) | 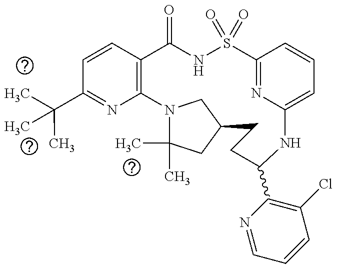 [SMILES: CC(C)(C)c1ccc2c(n1)N1C[C@@H](CCC(c3ncccc3Cl)Nc3cccc(n3)S(=O)(=O)NC2=O)CC1(C)C] | 2.09 | 582.218 | 583.2 | LC method A |
| Compound 285 Diastereomer 1) | 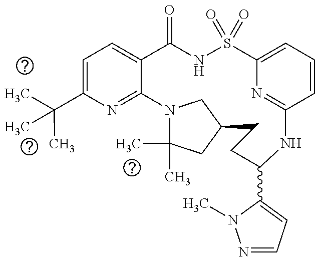 [SMILES: Cn1nccc1C1CC[C@@H]2CN(c3nc(C(C)(C)C)ccc3C(=O)NS(=O)(=O)c3cccc(n3)N1)C(C)(C)C2] | 1.92 | 551.268 | 552.2 | LC method A |
| Compound 284 (Diastereomer 2) | 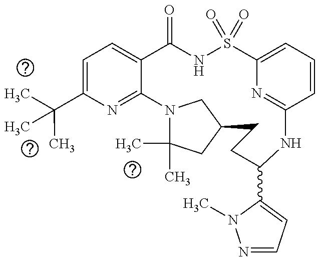 [SMILES: Cn1nccc1C1CC[C@@H]2CN(c3nc(C(C)(C)C)ccc3C(=O)NS(=O)(=O)c3cccc(n3)N1)C(C)(C)C2] | 1.90 | 551.268 | 552.1 | LC method A |
| Compound 245 (Diastereomer 1) | 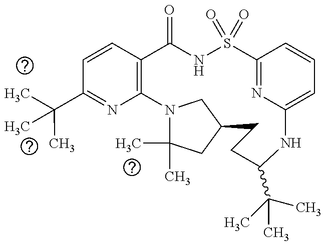 [SMILES: CC(C)(C)c1ccc2c(n1)N1C[C@@H](CCC(C(C)(C)C)Nc3cccc(n3)S(=O)(=O)NC2=O)CC1(C)C] | 2.34 | 527.293 | 528.2 | LC method A |
| Compound 244 (Diastereomer 2) | 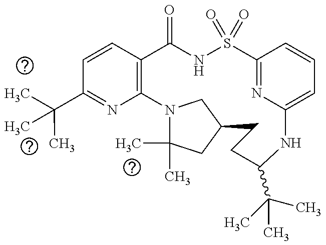 [SMILES: CC(C)(C)c1ccc2c(n1)N1C[C@@H](CCC(C(C)(C)C)Nc3cccc(n3)S(=O)(=O)NC2=O)CC1(C)C] | 2.30 | 527.293 | 528.2 | LC method A |

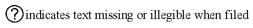

| Compound | | |
|---|---|---|
| Number | Structure | NMR |
| Compound 282 (diastereomer 2), hydrochloride salt | 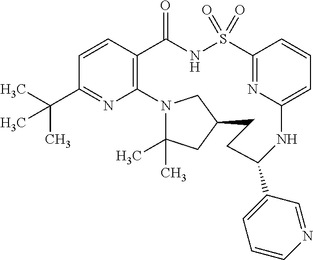 [SMILES: CC(C)(C)c1ccc2c(n1)N1C[C@@H](CC[C@@H](c3cccnc3)Nc3cccc(n3)S(=O)(=O)NC2=O)CC1(C)C] | ¹H NMR (500 MHz, DMSO-d₆) δ 12.51 (s, 1H), 9.01 (s, 1H), 8.81 (d, J = 5.6 Hz, 1H), 8.64 (d, J = 8.0 Hz, 1H), 8.05 (dd, J = 8.2, 4.4 Hz, 2H), 7.73-7.67 (m, 2H), 7.16 (d, J = 7.2 Hz, 1H), 6.90 (d, J = 8.5 Hz, 1H), 6.67 (d, J = 8.0 Hz, 1H), 5.37 (t, J = 11.2 Hz, 1H), 3.29 (d, J = 8.3 Hz, 1H), 2.72 (t, J = 10.2 Hz, 1H), 2.33 (t, J = 7.7 Hz, 1H), 2.04 (dt, J = 13.5, 7.6 Hz, 1H), 1.84 (dd, J = 11.6, 5.1 Hz, 2H), 1.77 (dd, J = 15.6, 6.1 Hz, 1H), 1.67 (s, 3H), 1.58 (t, J = 12.3 Hz, 1H), 1.51 (s, 3H), 1.50-1.43 (m, 1H), 1.29 (s, 9H). |
| Compound 279 (diastereomer 1) | 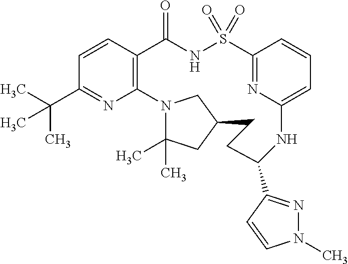 [SMILES: Cn1ccc([C@@H]2CC[C@@H]3CN(c4nc(C(C)(C)C)ccc4C(=O)NS(=O)(=O)c4cccc(n4)N2)C(C)(C)C3)n1] | ¹H NMR (500 MHz, DMSO-d₆) δ 12.54 (s, 1H), 7.60 (d, J = 8.2 Hz, 2H), 7.49 (d, J = 28.6 Hz, 2H), 7.08 (d, J = 7.1 Hz, 1H), 6.80 (d, J = 8.5 Hz, 1H), 6.65 (d, J = 7.9 Hz, 1H), 6.08 (s, 1H), 5.23 (s, 1H), 3.75 (s, 3H), 3.15 (s, 1H), 2.75 (d, J = 12.1 Hz, 1H), 2.19 (s, 1H), 1.97-1.77 (m, 3H), 1.65 (s, 4H), 1.53 (d, J = 24.0 Hz, 4H), 1.42 (s, 1H), 1.28 (s, 9H). |
| Compound 278 (diastereomer 2) | 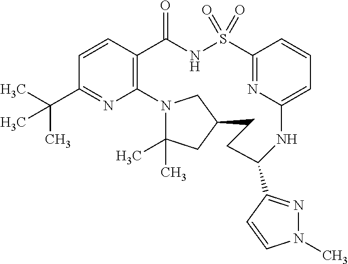 [SMILES: Cn1ccc([C@@H]2CC[C@@H]3CN(c4nc(C(C)(C)C)ccc4C(=O)NS(=O)(=O)c4cccc(n4)N2)C(C)(C)C3)n1] | ¹H NMR (500 MHz, DMSO-d₆) δ 12.34 (s, 1H), 7.64-7.41 (m, 4H), 7.20 (s, 1H), 6.77 (d, J = 8.4 Hz, 1H), 6.64 (s, 1H), 6.36 (s, 1H), 4.72 (s, 1H), 3.79 (s, 3H), 3.18 (s, 2H), 2.27 (d, J = 12.7 Hz, 1H), 1.95-1.78 (m, 2H), 1.71 (s, 1H), 1.61 (s, 3H), 1.53 (s, 4H), 1.28 (s, 10H), 1.17 (s, 1H). |
| Compound 233 (diastereomer 1), hydrochloride salt | 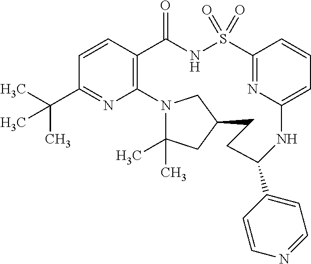 [SMILES: CC(C)(C)c1ccc2c(n1)N1C[C@@H](CC[C@@H](c3ccncc3)Nc3cccc(n3)S(=O)(=O)NC2=O)CC1(C)C] | ¹H NMR (500 MHz, DMSO-d₆) δ 12.43 (s, 1H), 8.89 (d, J = 6.2 Hz, 2H), 8.30 (s, 1H), 8.16 (d, J = 6.9 Hz, 2H), 7.70 (dd, J = 8.5, 7.3 Hz, 1H), 7.47 (d, J = 7.8 Hz, 1H), 7.29 (d, J = 7.3 Hz, 1H), 6.94 (d, J = 8.5 Hz, 1H), 6.63 (d, J = 7.9 Hz, 1H), 5.04 (s, 1H), 3.18 (s, 1H), 2.77 (s, 1H), 2.32 (s, 1H), 2.01 (s, 1H), 1.94-1.73 (m, 2H), 1.55 (d, J = 5.0 Hz, 6H), 1.50 (d, J = 11.6 Hz, 1H), 1.42-1.27 (m, 1H), 1.26 (s, 9H), 1.16 (d, J = 27.6 Hz, 1H) |
| Compound 232 (diastereomer 2), hydrochloride salt | 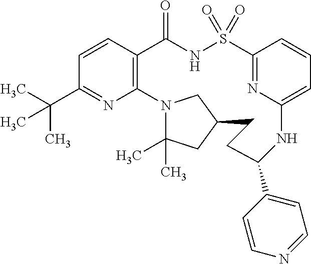 [SMILES: CC(C)(C)c1ccc2c(n1)N1C[C@@H](CC[C@@H](c3ccncc3)Nc3cccc(n3)S(=O)(=O)NC2=O)CC1(C)C] | ¹H NMR (500 MHz, DMSO-d₆) δ 12.54 (s, 1H), 8.97-8.87 (m, 2H), 8.28 (d, J = 8.8 Hz, 1H), 8.23-8.14 (m, 2H), 7.71 (dd, J = 8.5, 7.2 Hz, 1H), 7.65 (d, J = 7.9 Hz, 1H), 7.17 (d, J = 7.2 Hz, 1H), 6.97 (d, J = 8.3 Hz, 1H), 6.66 (d, J = 7.9 Hz, 1H), 5.41-5.36 (m, 1H), 3.27 (dd, J = 9.9, 6.8 Hz, 1H), 2.72 (t, J = 10.3 Hz, 1H), 2.36 (s, 1H), 1.99 (td, J = 13.4, 5.8 Hz, 1H), 1.80 (dq, J = 35.0, 6.7, 5.9 Hz, 3H), 1.66 (s, 3H), 1.56 (t, J = 12.4 Hz, 1H), 1.50 (s, 3H), 1.46 (d, J = 12.3 Hz, 1H), 1.31 (d, J = 11.6 Hz, 1H), 1.28 (s, 9H). |
| Compound 217 (diastereomer 1) | 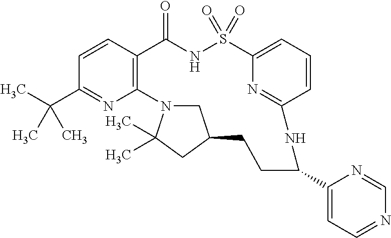 [SMILES: CC(C)(C)c1ccc2c(n1)N1C[C@@H](CC[C@@H](c3ccncn3)Nc3cccc(n3)S(=O)(=O)NC2=O)CC1(C)C] | ¹H NMR (500 MHz, DMSO-d₆) δ 12.50 (s, 1H), 9.15 (s, 1H), 8.76 (d, J = 5.2 Hz, 1H), 7.84 (d, J = 9.0 Hz, 1H), 7.69 (t, J = 7.8 Hz, 1H), 7.62 (d, J = 8.0 Hz, 1H), 7.58 (d, J = 5.3 Hz, 1H), 7.15 (d, J = 7.2 Hz, 1H), 6.94 (d, J = 8.5 Hz, 1H), 6.66 (d, J = 8.1 Hz, 1H), 5.23 (t, J = 10.3 Hz, 1H), 3.18 (dt, J = 7.6, 4.6 Hz, 1H), 2.75 (t, J = 10.4 Hz, 1H), 2.27 (d, J = 22.9 Hz, 1H), 1.99 (t, J = 13.0 Hz, 1H), 1.88 (ddd, J = 24.4, 12.4, 5.6 Hz, 2H), 1.76 (dd, J = 14.4, 5.7 Hz, 1H), 1.65 (s, 3H), 1.62-1.53 (m, 1H), 1.51 (s, 3H), 1.45 (dd, J = 25.5, 13.7 Hz, 1H), 1.28 (s, 9H). |
| Compound 207 (diastereomer 1) | 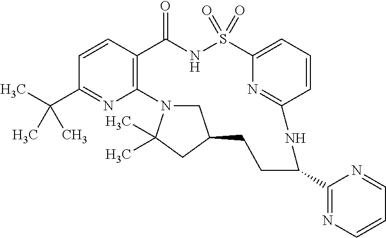 [SMILES: CC(C)(C)c1ccc2c(n1)N1C[C@@H](CC[C@@H](c3ncccn3)Nc3cccc(n3)S(=O)(=O)NC2=O)CC1(C)C] | ¹H NMR (500 MHz, DMSO-d₆) δ 12.56 (s, 1H), 8.77 (d, J = 4.8 Hz, 2H), 7.60 (d, J = 8.0 Hz, 3H), 7.38 (t, J = 4.9 Hz, 1H), 7.06 (s, 1H), 6.89 (s, 1H), 6.63 (d, J = 8.1 Hz, 1H), 5.43 (t, J = 11.0 Hz, 1H), 3.27 (s, 1H), 2.86 (s, 1H), 2.22 (s, 1H), 1.96 (t, J = 12.7 Hz, 1H), 1.87 (ddd, J = 22.4, 11.9, 5.2 Hz, 2H), 1.72 (d, J = 14.3 Hz, 1H), 1.65 (s, 3H), 1.52 (s, 5H), 1.29 (s, 9H). . |
| Compound 206 (diastereomer 2) | 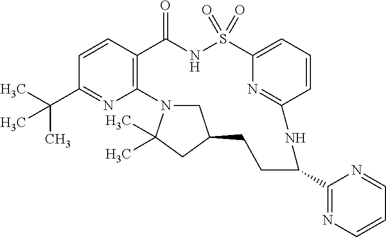 [SMILES: CC(C)(C)c1ccc2c(n1)N1C[C@@H](CC[C@@H](c3ncccn3)Nc3cccc(n3)S(=O)(=O)NC2=O)CC1(C)C] | ¹H NMR (500 MHz, DMSO-d₆) δ 12.26 (s, 1H), 8.84 (d, J = 4.9 Hz, 1H), 7.59 (s, 2H), 7.44 (t, J = 4.9 Hz, 1H), 7.27 (s, 2H), 7.19 (d, J = 7.3 Hz, 1H), 6.92 (d, J = 8.8 Hz, 1H), 6.61 (s, 1H), 5.16 (s, 1H), 3.08 (s, 1H), 2.28 (t, J = 7.5 Hz, 1H), 2.18 (s, 1H), 2.00 (s, 1H), 1.84 (dd, J = 12.0, 6.1 Hz, 1H), 1.65 (s, 3H), 1.57 (d, J = 11.4 Hz, 1H), 1.51 (s, 3H), 1.39- 1.31 (m, 1H), 1.28 (s, 10H), 0.85 (t, J = 6.6 Hz, 1H). |
| Compound 95 (diastereomer 1) | 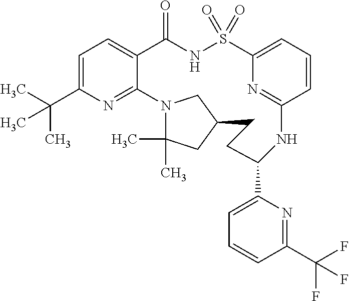 [SMILES: CC(C)(C)c1ccc2c(n1)N1C[C@@H](CC[C@@H](c3cccc(C(F)(F)F)n3)Nc3cccc(n3)S(=O)(=O)NC2=O)CC1(C)C] | ¹H NMR (400 MHz, Chloroform-d) δ 11.91 (s, 1H), 8.15 (d, J = 8.1 Hz, 1H), 7.90 (t, J = 7.9 Hz, 1H), 7.63 (d, J = 7.8 Hz, 3H), 7.52 (d, J = 7.3 Hz, 1H), 7.04 (s, 1H), 6.80 (s, 1H), 6.66 (s, 1H), 4.89 (s, 1H), 3.42 (s, 1H), 3.26 (t, J = 9.6 Hz, 1H), 2.59 (d, J = 14.2 Hz, 1H), 2.33 (s, 1H), 2.02 (d, J = 29.9 Hz, 1H), 1.83 (dq, J = 21.7, 12.0, 10.8 Hz, 4H), 1.59 (s, 3H), 1.56 (s, 3H), 1.32 (s, 9H). |
| Compound 94 (diastereomer 2) | 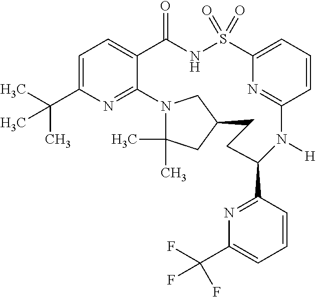 [SMILES: CC(C)(C)c1ccc2c(n1)N1C[C@@H](CC[C@H](c3cccc(C(F)(F)F)n3)Nc3cccc(n3)S(=O)(=O)NC2=O)CC1(C)C] | ¹H NMR (400 MHz, Chloroform-d) δ 10.52 (s, 1H), 7.95 (d, J = 8.0 Hz, 1H), 7.82 (t, J = 7.8 Hz, 1H), 7.56 (dd, J = 14.5, 8.2 Hz, 4H), 6.93-6.81 (m, 1H), 6.70 (d, J = 8.0 Hz, 1H), 5.85 (d, J = 8.6 Hz, 1H), 5.47 (s, 1H), 3.40 (s, 1H), 3.14-2.90 (m, 2H), 2.20 (s, 1H), 1.92 (s, 1H), 1.77 (s, 1H), 1.63 (s, 6H), 1.54 (dd, J = 12.4, 8.9 Hz, 2H), 1.31 (s, 9H), 1.27 (d, J = 10.1 Hz, 1H). |
| Compound 93 (diastereomer 1) | 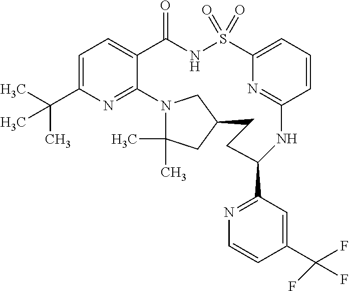 [SMILES: CC(C)(C)c1ccc2c(n1)N1C[C@@H](CC[C@H](c3cc(C(F)(F)F)ccn3)Nc3cccc(n3)S(=O)(=O)NC2=O)CC1(C)C] | ¹H NMR (400 MHz, Chloroform-d) δ 8.75 (d, J = 5.0 Hz, 1H), 7.92 (s, 1H), 7.54 (d, J = 12.3 Hz, 3H), 7.43 (d, J = 5.0 Hz, 1H), 6.84 (s, 1H), 6.67 (d, J = 7.9 Hz, 1H), 5.77 (s, 1H), 5.49 (q, J = 7.2 Hz, 1H), 3.42 (s, 1H), 3.00 (d, J = 8.8 Hz, 1H), 2.19 (d, J = 12.6 Hz, 1H), 1.93 (s, 1H), 1.74 (s, 1H), 1.64 (s, 3H), 1.63 (s, 3H), 1.60-1.42 (m, 3H), 1.31 (s, 9H), 1.27 (d, J = 7.9 Hz, 1H). |
| Compound 92 (diastereomer 2) | 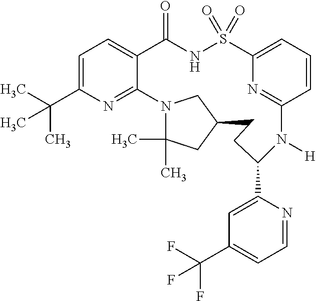 [SMILES: CC(C)(C)c1ccc2c(n1)N1C[C@@H](CC[C@@H](c3cc(C(F)(F)F)ccn3)Nc3cccc(n3)S(=O)(=O)NC2=O)CC1(C)C] | ¹H NMR (400 MHz, Chloroform-d) δ 12.00 (s, 1H), 8.76 (d, J = 5.0 Hz, 1H), 8.15 (d, J = 8.1 Hz, 1H), 7.66 (dd, J = 17.5, 7.6 Hz, 2H), 7.54-7.42 (m, 2H), 7.06 (d, J = 8.1 Hz, 1H), 6.78 (d, J = 8.2 Hz, 1H), 6.71 (s, 1H), 4.86 (s, 1H), 3.42 (s, 1H), 3.25 (s, 1H), 2.70- 2.53 (m, 1H), 2.35 (s, 1H), 2.11 (d, J = 11.2 Hz, 1H), 2.00 (s, 1H), 1.81 (q, J = 14.3, 10.5 Hz, 1H), 1.61 (s, 3H), 1.57 (s, 3H), 1.45 (d, J = 26.6 Hz, 1H), 1.32 (s, 9H), 1.23 (d, J = 19.3 Hz, 1H). |
| Compound 41 (diastereomer 1) | 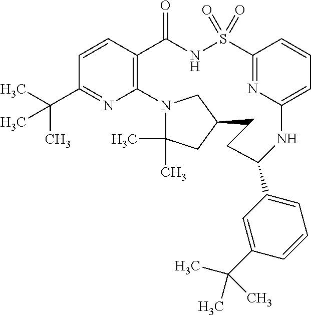 [SMILES: CC(C)(C)c1cccc([C@@H]2CC[C@@H]3CN(c4nc(C(C)(C)C)ccc4C(=O)NS(=O)(=O)c4cccc(n4)N2)C(C)(C)C3)c1] | ¹H NMR (400 MHz, DMSO-d₆) δ 12.43 (s, 1H), 7.68-7.54 (m, 3H), 7.45 (s, 1H), 7.26- 7.13 (m, 3H), 7.06 (d, J = 7.2 Hz, 1H), 6.80 (d, J = 8.5 Hz, 1H), 6.66 (d, J = 8.0 Hz, 1H), 5.23-5.13 (m, 1H), 3.23 (dd, J = 10.3, 6.9 Hz, 1H), 2.77 (t, J = 10.4 Hz, 1H), 2.31- 2.18 (m, 1H), 1.92 (td, J = 13.4, 5.7 Hz, 1H), 1.83 (dd, J = 11.6, 5.1 Hz, 1H), 1.76 (d, J = 13.8 Hz, 1H), 1.73-1.68 (m, 1H), 1.65 (s, 3H), 1.56 (t, J = 12.4 Hz, 1H), 1.51 (s, 3H), 1.49-1.40 (m, 1H), 1.28 (s, 9H), 1.25 (s, 9H) |
| Compound 40 (diastereomer 2) | 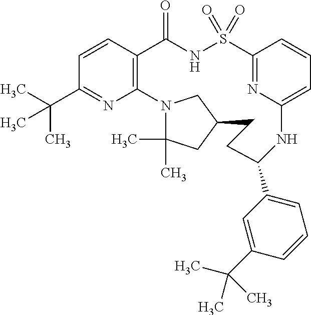 [SMILES: CC(C)(C)c1cccc([C@@H]2CC[C@@H]3CN(c4nc(C(C)(C)C)ccc4C(=O)NS(=O)(=O)c4cccc(n4)N2)C(C)(C)C3)c1] | ¹H NMR (400 MHz, DMSO-d₆) δ 12.21 (s, 1H), 7.65-7.55 (m, 2H), 7.54-7.42 (m, 2H), 7.35-7.27 (m, 1H), 7.25 (s, 2H), 7.17 (d, J = 7.3 Hz, 1H), 6.77 (d, J = 8.5 Hz, 1H), 6.64 (d, J = 7.9 Hz, 1H), 5.01-4.72 (m, 1H), 3.27-3.02 (m, 2H), 2.43-2.15 (m, 2H), 1.98-1.87 (m, 1H), 1.85 (dd, J = 11.8, 5.7 Hz, 1H), 1.76-1.67 (m, 1H), 1.60 (s, 3H), 1.53 (s, 3H), 1.52-1.42 (m, 1H), 1.28 (s, 9H), 1.27 (s, 9H), 1.21-1.03 (m, 1H) |
| Compound 38 (diastereomer 1) | 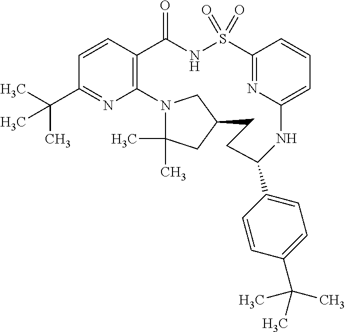 [SMILES: CC(C)(C)c1ccc([C@@H]2CC[C@@H]3CN(c4nc(C(C)(C)C)ccc4C(=O)NS(=O)(=O)c4cccc(n4)N2)C(C)(C)C3)cc1] | ¹H NMR (400 MHz, DMSO-d₆) δ 12.44 (s, 1H), 7.70-7.55 (m, 3H), 7.36-7.24 (m, 4H), 7.05 (d, J = 7.2 Hz, 1H), 6.79 (d, J = 8.5 Hz, 1H), 6.66 (d, J = 8.0 Hz, 1H), 5.17 (t, J = 11.0 Hz, 1H), 3.23 (dd, J = 10.3, 6.9 Hz, 1H), 2.78 (t, J = 10.4 Hz, 1H), 2.30- 2.18 (m, 1H), 1.92 (dt, J = 13.3, 7.0 Hz, 1H), 1.83 (dd, J = 11.8, 5.2 Hz, 1H), 1.71 (dd, J = 14.5, 8.3 Hz, 2H), 1.65 (s, 3H), 1.57 (t, J = 12.4 Hz, 1H), 1.51 (s, 3H), 1.45 (d, J = 11.9 Hz, 1H), 1.28 (s, 9H), 1.24 (s, 9H) |
| Compound 37 (diastereomer 2) | 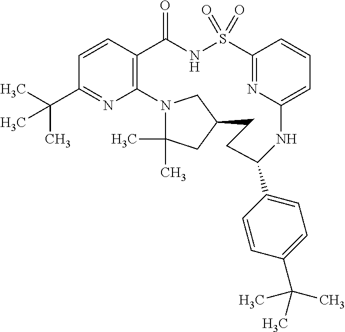 [SMILES: CC(C)(C)c1ccc([C@@H]2CC[C@@H]3CN(c4nc(C(C)(C)C)ccc4C(=O)NS(=O)(=O)c4cccc(n4)N2)C(C)(C)C3)cc1] | ¹H NMR (400 MHz, DMSO-d₆) δ 12.19 (s, 1H), 7.58 (t, J = 7.8 Hz, 2H), 7.56-7.47 (m, 1H), 7.42 (d, J = 8.1 Hz, 2H), 7.34 (d, J = 7.9 Hz, 2H), 7.16 (d, J = 7.2 Hz, 1H), 6.74 (d, J = 8.5 Hz, 1H), 6.65 (d, J = 8.0 Hz, 1H), 4.97-4.74 (m, 1H), 3.27-3.04 (m, 2H), 2.35-2.27 (m, 1H), 2.26-2.05 (m, 1H), 1.98-1.87 (m, 1H), 1.84 (dd, J = 11.8, 5.8 Hz, 1H), 1.76-1.64 (m, 1H), 1.61 (s, 3H), 1.52 (s, 3H), 1.51-1.45 (m, 1H), 1.28 (s, 9H), 1.26 (s, 9H), 1.16-1.01 (m, 1H) |
| Compound 81 (diastereomer 2) | 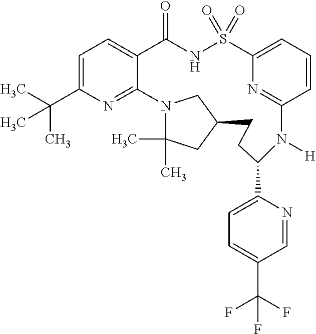 [SMILES: CC(C)(C)c1ccc2c(n1)N1C[C@@H](CC[C@@H](c3ccc(C(F)(F)F)cn3)Nc3cccc(n3)S(=O)(=O)NC2=O)CC1(C)C] | ¹H NMR (400 MHz, Chloroform-d) δ 8.84 (s, 1H), 8.15 (s, 1H), 7.94 (s, 1H), 7.64 (s, 1H), 7.49 (d, J = 26.1 Hz, 1H), 7.05 (s, 1H), 6.74 (d, J = 25.9 Hz, 2H), 4.88 (s, 1H). 3.42 (s, 1H), 3.26 (s, 1H), 2.61 (s, 1H), 2.31 (s, 1H), 2.12-1.66 (m, 6H), 1.58 (s, 6H), 1.31 (s, 9H). |
| Compound 80 (diastereomer 1) | 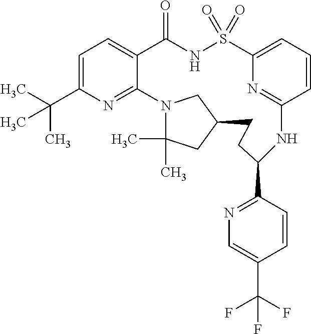 [SMILES: CC(C)(C)c1ccc2c(n1)N1C[C@@H](CC[C@H](c3ccc(C(F)(F)F)cn3)Nc3cccc(n3)S(=O)(=O)NC2=O)CC1(C)C] | ¹H NMR (400 MHz, Chloroform-d) δ 8.81 (s, 1H), 7.92 (d, J = 7.8 Hz, 1H), 7.86 (d, J = 8.1 Hz, 1H), 7.56 (d, J = 11.5 Hz, 2H), 7.45 (d, J = 8.0 Hz, 1H), 6.84 (s, 1H), 6.67 (d, J = 8.0 Hz, 1H), 5.82 (s, 1H), 5.47 (d, J = 8.1 Hz, 1H), 3.42 (s, 1H), 3.00 (s, 2H), 2.18 (s, 1H), 1.90 (s, 1H), 1.78 (s, 2H), 1.63 (s, 3H), 1.62 (s, 3H), 1.55 (t, J = 10.9 Hz, 2H), 1.31 (s, 9H). |
| Compound 66 (diastereomer 1) | 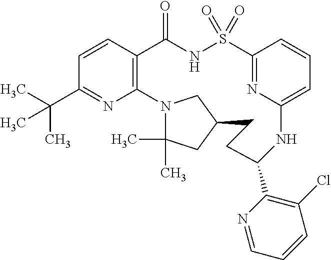 [SMILES: CC(C)(C)c1ccc2c(n1)N1C[C@@H](CC[C@@H](c3ncccc3Cl)Nc3cccc(n3)S(=O)(=O)NC2=O)CC1(C)C] | ¹H NMR (400 MHz, DMSO-d₆) δ 12.15 (s, 1H), 8.61 (d, J = 4.6 Hz, 1H), 7.96 (dd, J = 8.1, 1.5 Hz, 1H), 7.61 (t, J = 7.9 Hz, 1H), 7.51 (d, J = 9.1 Hz, 2H), 7.41 (dd, J = 8.1, 4.7 Hz, 1H), 7.21 (d, J = 6.8 Hz, 1H), 6.91 (d, J = 8.5 Hz, 1H), 6.65 (d, J = 8.0 Hz, 1H), 5.75-5.62 (m, 1H), 3.51 (s, 1H), 3.32 (s, 1H), 3.03 (s, 1H), 2.33 (s, 1H), 2.27-2.10 (m, 1H), 1.82 (dd, J = 12.0, 6.0 Hz, 1H), 1.70 (s, 2H), 1.66 (s, 3H), 1.60 (s, 1H), 1.50 (s, 3H), 1.28 (s, 9H). |
| Compound 65 (diastereomer 2) | 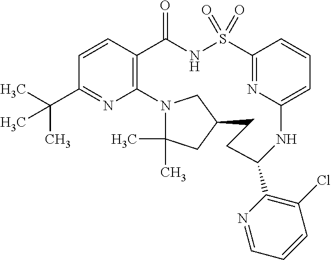 [SMILES: CC(C)(C)c1ccc2c(n1)N1C[C@@H](CC[C@@H](c3ncccc3Cl)Nc3cccc(n3)S(=O)(=O)NC2=O)CC1(C)C] | ¹H NMR (400 MHz, DMSO-d₆) δ 12.47 (s, 1H), 8.59-8.43 (m, 1H), 7.89 (dd, J = 8.1, 1.6 Hz, 1H), 7.74 (d, J = 9.5 Hz, 1H), 7.67- 7.56 (m, 2H), 7.33 (dd, J = 8.1, 4.6 Hz, 1H), 7.07 (d, J = 7.2 Hz, 1H), 6.91 (d, J = 8.5 Hz, 1H), 6.67 (d, J = 8.0 Hz, 1H), 5.79 (t, J = 10.6 Hz, 1H), 3.32 (t, J = 8.6 Hz, 1H), 2.86 (t, J = 10.4 Hz, 1H), 2.22 (dt, J = 12.1, 4.9 Hz, 1H), 1.88 (ddd, J = 17.6, 12.6, 5.8 Hz, 2H), 1.74 (dd, J = 14.6, 4.6 Hz, 1H), 1.62 (s, 3H), 1.61-1.53 (m, 2H), 1.52 (s, 3H), 1.46 (d, J = 13.2 Hz, 1H), 1.29 (s, 9H). |
| Compound 285 (Diastereomer 1) | 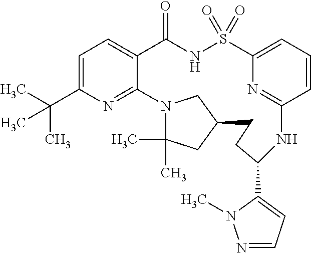 [SMILES: Cn1nccc1[C@@H]1CC[C@@H]2CN(c3nc(C(C)(C)C)ccc3C(=O)NS(=O)(=O)c3cccc(n3)N1)C(C)(C)C2] | ¹H NMR (500 MHz, DMSO-d₆) δ 12.51 (s, 1H), 7.71-7.61 (m, 3H), 7.27 (d, J = 2.1 Hz, 1H), 7.12 (d, J = 7.2 Hz, 1H), 6.76 (d, J = 8.5 Hz, 1H), 6.69 (d, J = 8.0 Hz, 1H), 6.19 (d, J = 2.0 Hz, 1H), 5.28 (t, J = 11.1 Hz, 1H), 3.90 (s, 3H), 3.19 (t, J = 8.5 Hz, 1H), 2.78 (t, J = 10.3 Hz, 1H), 2.27 (s, 1H), 2.02 (q, J = 10.4, 9.1 Hz, 1H), 1.83 (dd, J = 11.9, 5.3 Hz, 1H), 1.77-1.69 (m, 2H), 1.63 (s, 3H), 1.57 (t, J = 12.3 Hz, 1H), 1.51 (s, 3H), 1.49-1.41 (m, 1H), 1.29 (s, 9H). |
| Compound 284 (Diastereomer 2) | 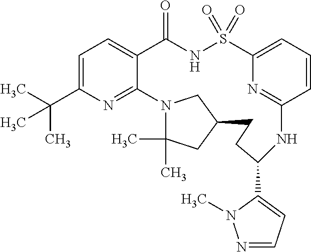 [SMILES: Cn1nccc1[C@@H]1CC[C@@H]2CN(c3nc(C(C)(C)C)ccc3C(=O)NS(=O)(=O)c3cccc(n3)N1)C(C)(C)C2] | ¹H NMR (500 MHz, DMSO-d₆) δ 12.25 (s, 1H), 7.72-7.57 (m, 2H), 7.51 (s, 1H), 7.31 (s, 1H), 7.22 (d, J = 6.8 Hz, 1H), 6.74 (d, J = 8.4 Hz, 1H), 6.66 (d, J = 7.8 Hz, 1H), 6.40 (s, 1H), 5.04 (s, 1H), 3.83 (s, 3H), 3.16 (d, J = 17.4 Hz, 2H), 2.51 (s, 2H), 2.31 (s, 1H), 1.94 (s, 1H), 1.84 (dd, J = 12.3, 5.9 Hz, 2H), 1.60 (s, 3H), 1.54 (s, 3H), 1.28 (s, 9H), 1.15 (s, 1H). |
| Compound 245 (Diastereomer 1) | 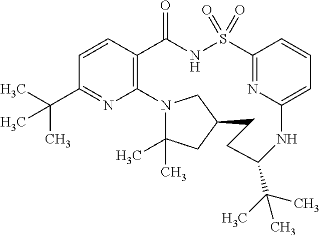 [SMILES: CC(C)(C)c1ccc2c(n1)N1C[C@@H](CC[C@@H](C(C)(C)C)Nc3cccc(n3)S(=O)(=O)NC2=O)CC1(C)C] | ¹H NMR (500 MHz, DMSO-d₆) δ 12.22 (s, 1H), 7.63-7.47 (m, 2H), 7.09 (d, J = 7.2 Hz, 1H), 6.79 (d, J = 8.5 Hz, 2H), 6.63 (d, J = 7.9 Hz, 1H), 3.92 (d, J = 21.2 Hz, 1H), 2.97 (s, 1H), 2.84 (s, 1H), 2.23 (d, J = 9.0 Hz, 1H), 1.82 (td, J = 15.1, 13.5, 6.8 Hz, 2H), 1.63 (d, J = 14.8 Hz, 6H), 1.52 (s, 3H), 1.45- 1.32 (m, 1H), 1.28 (s, 9H), 0.89 (s, 9H). |
| Compound 244 (Diastereomer 2) | 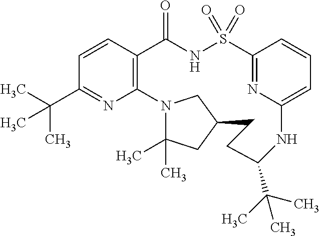 [SMILES: CC(C)(C)c1ccc2c(n1)N1C[C@@H](CC[C@@H](C(C)(C)C)Nc3cccc(n3)S(=O)(=O)NC2=O)CC1(C)C] | ¹H NMR (500 MHz, DMSO-d₆) δ 12.41 (s, 1H), 7.62 (d, J = 8.0 Hz, 1H), 7.55 (dd, J = 8.5, 7.2 Hz, 1H), 7.01 (dd, J = 7.1, 0.7 Hz, 1H), 6.88 (d, J = 9.1 Hz, 1H), 6.76 (dd, J = 8.5, 0.8 Hz, 1H), 6.65 (d, J = 7.9 Hz, 1H), 3.84 (ddd, J = 11.7, 9.0, 2.2 Hz, 1H), 3.15- 3.03 (m, 1H), 2.65 (t, J = 10.5 Hz, 1H), 2.02 (d, J = 5.8 Hz, 1H), 1.80 (dd, J = 11.7, 5.3 Hz, 1H), 1.65 (dd, J = 13.7, 5.4 Hz, 1H), 1.62 (s, 3H), 1.60-1.55 (m, 1H), 1.52 (d, J = 12.4 Hz, 2H), 1.48 (s, 3H), 1.28 (s, 9H), 1.26-1.16 (m, 1H), 0.87 (s, 9H). |

### Example 13: Preparation of (14S)-8-tert-Butyl-17-(5-chloropyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione isomers, Compound 209 (diastereomer 1) and Compound 208 (diastereomer 2)

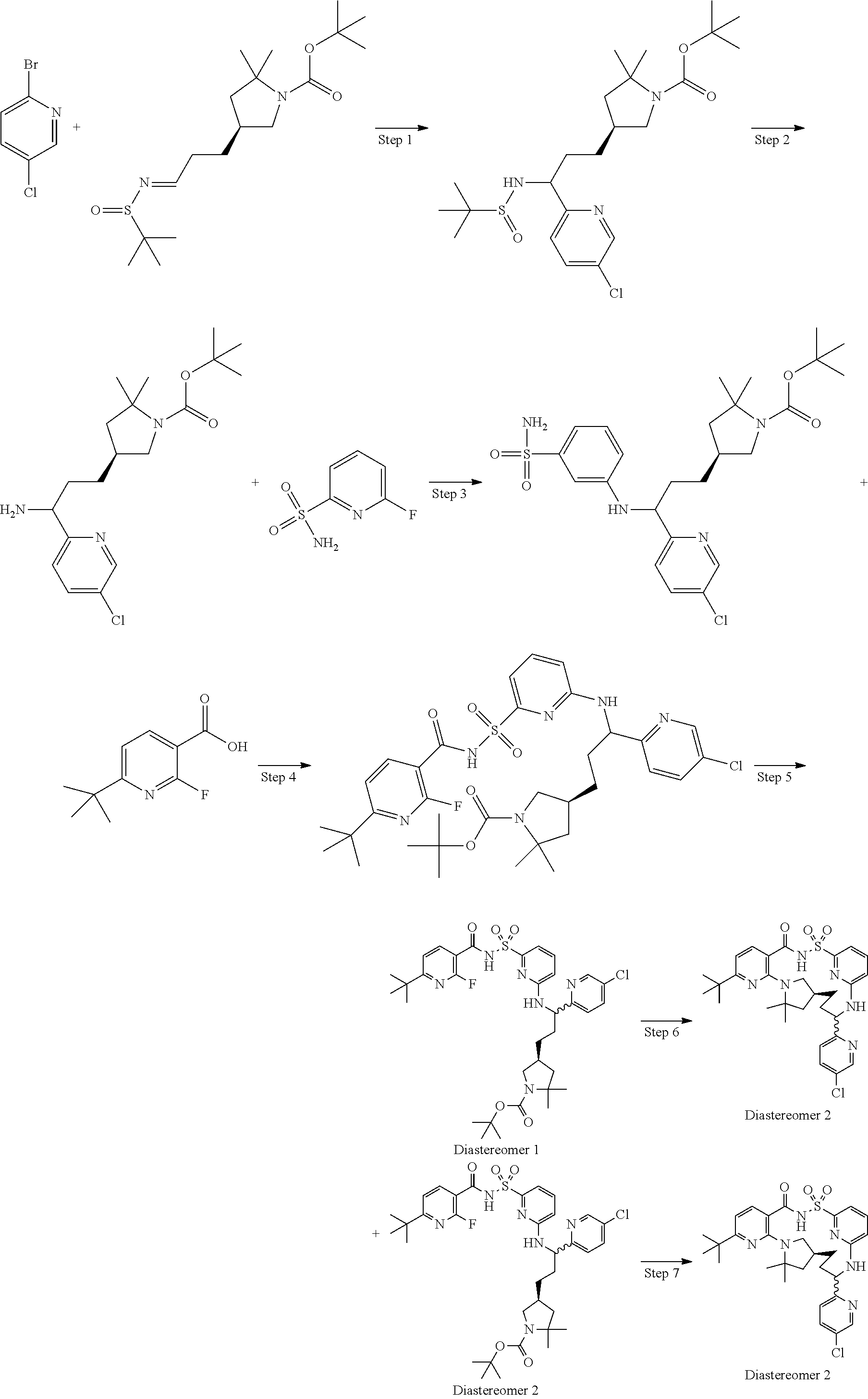

### Step 1: tert-Butyl (4S)-4-[3-(tert-butylsulfinylamino)-3-(5-chloro-2-pyridyl)propyl]-2,2-dimethyl-pyrrolidine-1-carboxylate

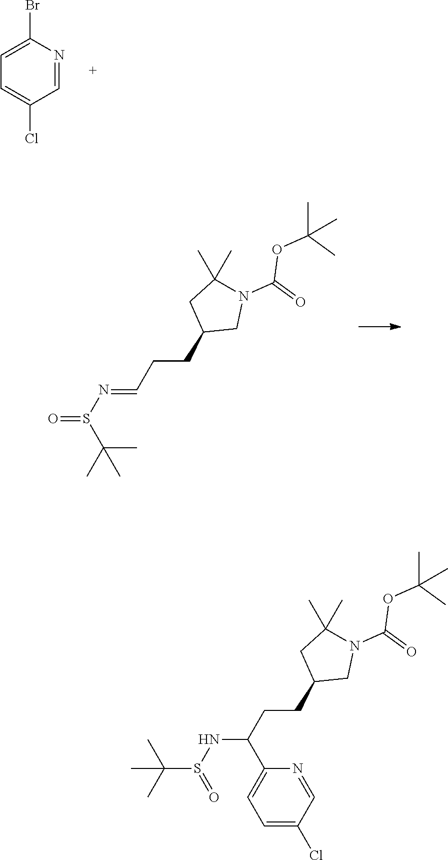

2-Bromo-5-chloro-pyridine (8.8 g, 44.814 mmol) was dissolved in diethyl ether (250 mL) and cooled in a dry ice acetone bath (<−70° C.) under a nitrogen balloon. The mixture was stirred for 15 min. n-BuLi (18 mL of 2.5 M in hexanes, 45.00 mmol) was added in quick dropwise fashion. The mixture was stirred at this temperature for 30 min. tert-Butyl(4S)-4-[3E)-3-tert-butylsulfinyliminopropyl]-2,2-dimethyl-pyrrolidine-1-carboxylate (4 g, 10.599 mmol) in THF (5 mL plus 1 mL rinse) was added in one portion. The mixture was stirred for another 15 min after addition. NH₄Cl (20 mL, saturated aqueous) was added. The mixture was taken out of dry ice acetone bath and diluted with EtOAc (200 mL) and water (100 mL). The layers were separated and the organic layer was filtered through an anhydrous Na₂SO₄ pad. The filtrate was concentrated and the residue was purified by silica gel chromatography (120 g column), using 10-80% EtOAc in hexanes to afford tert-butyl (4S)-4-[3-(tert-butylsulfinylamino)-3-(5-chloro-2-pyridyl)propyl]-2,2-dimethyl-pyrrolidine-1-carboxylate (2.31 g, 45%) as a white foam. ESI-MS m/z calc. 471.2322, found 472.7 (M+1)⁺; Retention time: 3.8 minutes (LC method B).

### Step 2: tert-Butyl (4S)-4-[3-amino-3-(5-chloro-2-pyridyl)propyl]-2,2-dimethyl-pyrrolidine-1-carboxylate

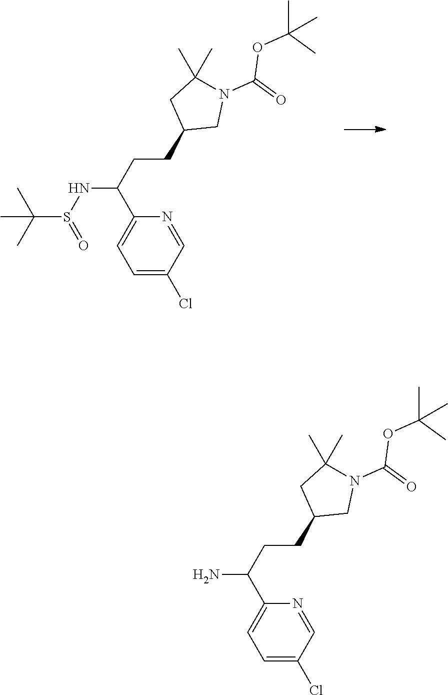 [SMILES: CC(C)(C)OC(=O)N1C[C@@H](CCC(NS(=O)C(C)(C)C)c2ccc(Cl)cn2)CC1(C)C ; CC(C)(C)OC(=O)N1C[C@@H](CCC(N)c2ccc(Cl)cn2)CC1(C)C]

tert-Butyl (4S)-4-[3-(tert-butylsulfinylamino)-3-(5-chloro-2-pyridyl)propyl]-2,2-dimethyl-pyrrolidine-1-carboxylate (8.95 g, 18.011 mmol) was dissolved in a solvent mixture of THF (100 mL) and water (20 mL). Molecular iodine (1.38 g, 5.437 mmol) was added in one portion. The mixture was placed in a 50° C. oil bath and stirred under a nitrogen balloon for 3 h. It was then cooled to rt, diluted with Na₂S₂O₃ (5 g in 20 mL of saturated aqueous sodium bicarbonate) and EtOAc (50 mL). The layers were separated and the organic layer was washed with brine (˜30 mL), dried over anhydrous MgSO₄, filtered and concentrated. This crude material was used in the next step without further purification. ESI-MS m/z calc. 367.20267, found 368.5 (M+1)⁺; Retention time: 2.91 minutes (LC method B).

### Step 3: tert-Butyl (4S)-4-[3-(5-chloro-2-pyridyl)-3-[(6-sulfamoyl-2-pyridyl)amino]propyl]-2,2-dimethyl-pyrrolidine-1-carboxylate

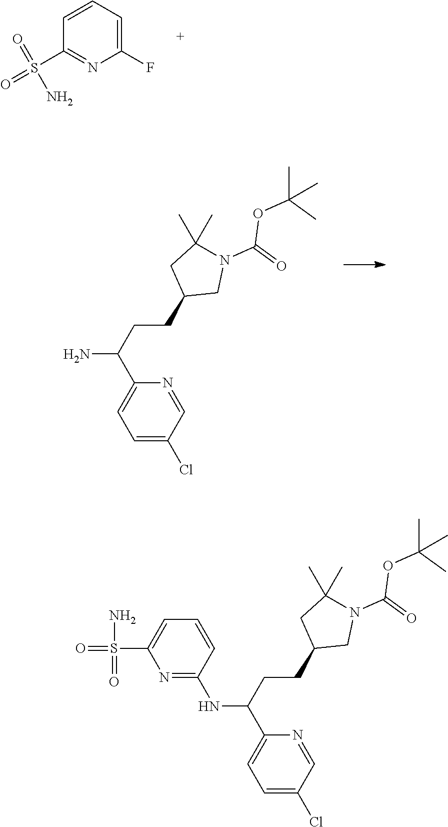 [SMILES: NS(=O)(=O)c1cccc(F)n1 ; CC(C)(C)OC(=O)N1C[C@@H](CCC(N)c2ccc(Cl)cn2)CC1(C)C ; CC(C)(C)OC(=O)N1C[C@@H](CCC(Nc2cccc(S(N)(=O)=O)n2)c2ccc(Cl)cn2)CC1(C)C]

tert-Butyl (4S)-4-[3-amino-3-(5-chloro-2-pyridyl)propyl]-2,2-dimethyl-pyrrolidine-1-carboxylate (4.73 g, 12.213 mmol) was dissolved in DMSO (6 mL). 6-Fluoropyridine-2-sulfonamide (2.5 g, 14.191 mmol) was added, followed by Na₂CO₃ (3.7 g, 34.910 mmol). The mixture was heated in a 110° C. oil bath under nitrogen balloon for 20 h. It was then cooled to rt and diluted with EtOAc (50 mL) and water (40 mL). The layers were separated and the organic layer was washed with more water (40 mL) and brine (30 mL). It was then dried over anhydrous Na₂SO₄, filtered and concentrated. The residue was purified by silica gel chromatography (80 g column), using 5-80% EtOAc in hexanes to afford tert-butyl (4S)-4-[3-(5-chloro-2-pyridyl)-3-[(6-sulfamoyl-2-pyridyl)amino]propyl]-2,2-dimethyl-pyrrolidine-1-carboxylate as a white foam (3.88 g, 58%). ¹H NMR (500 MHz, DMSO-d₆) δ 8.57 (d, J=2.5 Hz, 1H), 7.83 (dd, J=8.4, 2.6 Hz, 1H), 7.63-7.45 (m, 3H), 7.06 (s, 2H), 6.97 (d, J=7.2 Hz, 1H), 6.70 (d, J=8.5 Hz, 1H), 5.18 (s, 1H), 3.54 (dd, J=18.9, 10.0 Hz, 1H), 2.84-2.67 (m, 1H), 2.08 (d, J=6.0 Hz, 1H), 1.94-1.74 (m, 3H), 1.45-1.30 (m, 15H), 1.23 (s, 3H). ESI-MS m/z calc. 523.202, found 524.5 (M+1)⁺; Retention time: 2.69 minutes (LC method H).

### Step 4: tert-Butyl (4S)-4-[3-[[6-[(6-tert-butyl-2-fluoro-pyridine-3-carbonyl)sulfamoyl]-2-pyridyl]amino]-3-(5-chloro-2-pyridyl)propyl]-2,2-dimethyl-pyrrolidine-1-carboxylate

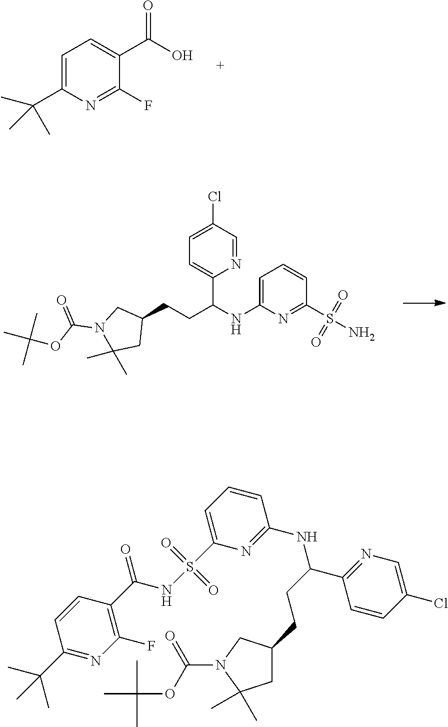 [SMILES: CC(C)(C)c1ccc(C(=O)O)c(F)n1 ; CC(C)(C)OC(=O)N1C[C@@H](CCC(Nc2cccc(S(N)(=O)=O)n2)c2ccc(Cl)cn2)CC1(C)C ; CC(C)(C)OC(=O)N1C[C@@H](CCC(Nc2cccc(S(=O)(=O)NC(=O)c3ccc(C(C)(C)C)nc3F)n2)c2ccc(Cl)cn2)CC1(C)C]

To a solution of 6-tert-butyl-2-fluoro-pyridine-3-carboxylic acid (773 mg, 3.920 mmol) in THF (15 mL) was added CDI (642 mg, 3.959 mmol) and the mixture was stirred at rt for 16 h then tert-butyl (4S)-4-[3-(5-chloro-2-pyridyl)-3-[(6-sulfamoyl-2-pyridyl)amino]propyl]-2,2-dimethyl-pyrrolidine-1-carboxylate (1.7 g, 3.244 mmol) was added followed by DBU (1.5 mL, 10.03 mmol) and the resulting mixture was stirred for 2 h at room temperature. The reaction was quenched with a 1:1 mixture of saturated ammonium chloride and brine solutions, then extracted with ethyl acetate. The combined organic layers was washed with brine, dried over sodium sulfate, filtered and evaporated. The resulting residue was used in the next step.

### Step 5: tert-Butyl (4S)-4-[3-[[6-[(6-tert-butyl-2-fluoro-pyridine-3-carbonyl)sulfamoyl]-2-pyridyl]amino]-3-(5-chloro-2-pyridyl)propyl]-2,2-dimethyl-pyrrolidine-1-carboxylate, diastereomer 1, and tert-butyl (4S)-4-[3-[[6-[(6-tert-butyl-2-fluoro-pyridine-3-carbonyl)sulfamoyl]-2-pyridyl]amino]-3-(5-chloro-2-pyridyl)propyl]-2,2-dimethyl-pyrrolidine-1-carboxylate, diastereomer 2

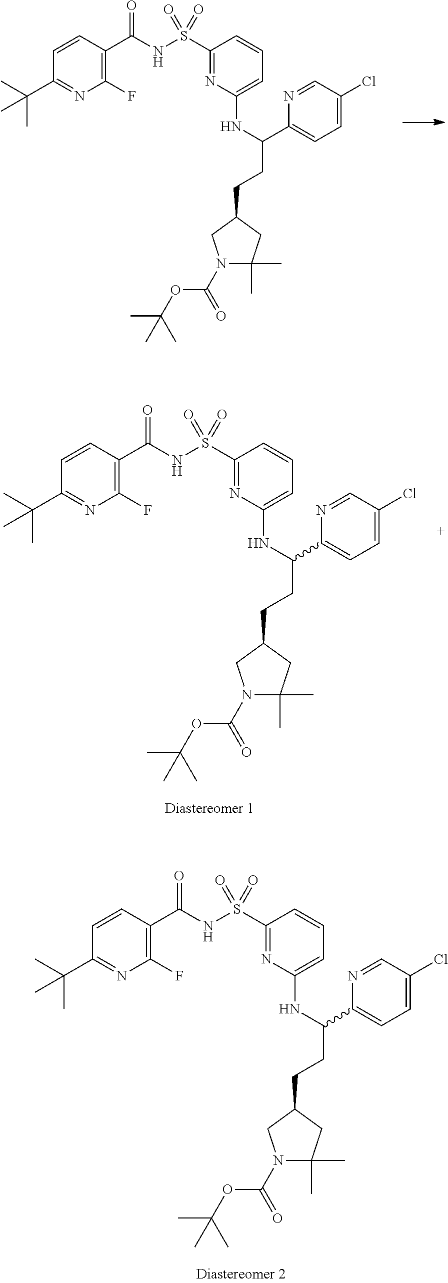 [SMILES: CC(C)(C)OC(=O)N1C[C@@H](CCC(Nc2cccc(S(=O)(=O)NC(=O)c3ccc(C(C)(C)C)nc3F)n2)c2ccc(Cl)cn2)CC1(C)C ; CC(C)(C)OC(=O)N1C[C@@H](CCC(Nc2cccc(S(=O)(=O)NC(=O)c3ccc(C(C)(C)C)nc3F)n2)c2ccc(Cl)cn2)CC1(C)C ; CC(C)(C)OC(=O)N1C[C@@H](CCC(Nc2cccc(S(=O)(=O)NC(=O)c3ccc(C(C)(C)C)nc3F)n2)c2ccc(Cl)cn2)CC1(C)C]

tert-Butyl (4S)-4-[3-[[6-[(6-tert-butyl-2-fluoro-pyridine-3-carbonyl)sulfamoyl]-2-pyridyl]amino]-3-(5-chloro-2-pyridyl)propyl]-2,2-dimethyl-pyrrolidine-1-carboxylate (2.38 g, 3.384 mmol) was purified by reverse phase chromatography with a gradient from 20% acetontrile in water to 100% water (˜500 mg in 2.5 mL per injection, totaling 5 injections) to afford as two isomers as white solids:

Diastereomer 1: tert-Butyl (4S)-4-[3-[[6-[(6-tert-butyl-2-fluoro-pyridine-3-carbonyl)sulfamoyl]-2-pyridyl]amino]-3-(5-chloro-2-pyridyl)propyl]-2,2-dimethyl-pyrrolidine-1-carboxylate (662 mg, 56%). ESI-MS m/z calc. 702.2767, found 703.2 (M+1)⁺; Retention time: 2.29 minutes (LC method A).

Diastereomer 2: tert-Butyl (4S)-4-[3-[[6-[(6-tert-butyl-2-fluoro-pyridine-3-carbonyl)sulfamoyl]-2-pyridyl]amino]-3-(5-chloro-2-pyridyl)propyl]-2,2-dimethyl-pyrrolidine-1-carboxylate (472 mg, 40%) ESI-MS m/z calc. 702.2767, found 703.5 (M+1)⁺; Retention time: 2.3 minutes (LC Method A).

### Step 6: (14S)-8-tert-Butyl-17-(5-chloropyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 209 (diastereomer 1)

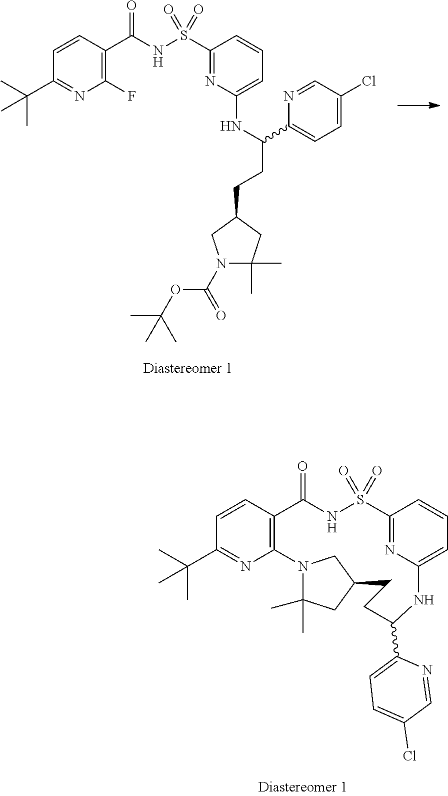 [SMILES: CC(C)(C)OC(=O)N1C[C@@H](CCC(Nc2cccc(S(=O)(=O)NC(=O)c3ccc(C(C)(C)C)nc3F)n2)c2ccc(Cl)cn2)CC1(C)C ; CC(C)(C)c1ccc2c(n1)N1C[C@@H](CCC(c3ccc(Cl)cn3)Nc3cccc(n3)S(=O)(=O)NC2=O)CC1(C)C]

tert-Butyl (4S)-4-[3-[[6-[(6-tert-butyl-2-fluoro-pyridine-3-carbonyl)sulfamoyl]-2-pyridyl]amino]-3-(5-chloro-2-pyridyl)propyl]-2,2-dimethyl-pyrrolidine-1-carboxylate (662 mg, 0.9413 mmol) (diastereomer 1) was dissolved in DCM (13.5 mL) and to the mixture was added TFA (3.25 mL, 42.18 mmol) and the reaction was stirred at room temperature. After 1 h, the mixture was evaporated to dryness, then diluted with diethyl ether (30 mL×2), and concentrated. The material was then placed on the high vacuum pump for 2 h to afford intermediate 6-tert-butyl-N-[[6-[[1-(5-chloro-2-pyridyl)-3-[(3S)-5,5-dimethylpyrrolidin-3-yl]propyl]amino]-2-pyridyl]sulfonyl]-2-fluoro-pyridine-3-carboxamide as an off-white solid. ESI-MS m/z calc. 602.22424, found 603.2 (M+1)⁺; Retention time: 1.37 minutes. The intermediate and K₂CO₃ (1.4 g, 10.13 mmol), 3 Å molecular sieves and NMP (12 mL) were combined in a vial, which was purged with nitrogen, capped, heated to 150° C. and stirred for 3 h, then 170° C. and stirred for 1 h. The mixture was diluted with ethyl acetate and water. The organic layer was extracted (2×) and was further washed with 10% citric acid solution followed by brine. The organics were separated, dried over sodium sulfate, evaporated to a light brown oil. The residue was purified separately by reverse-phase preparative chromatography utilizing a C₁₈ column and HPLC-MS method using a 30-99% over 15 min gradient of acetonitrile in water (+5 mM HCl), to afford the diastereomer product as a white solid: (14S)-8-tert-butyl-17-(5-chloropyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 1 (214.1 mg, 39%). ¹H NMR (500 MHz, DMSO-d₆) δ 12.30 (s, 1H), 8.69-8.53 (m, 1H), 7.90 (d, J=8.6 Hz, 1H), 7.78 (s, 1H), 7.66-7.57 (m, 2H), 7.50 (s, 1H), 7.23 (d, J=7.2 Hz, 1H), 6.87 (d, J=8.5 Hz, 1H), 6.64 (d, J=8.0 Hz, 1H), 4.99 (s, 1H), 3.91 (s, 2H), 3.11 (s, 1H), 2.32 (s, 1H), 2.14 (d, J=17.2 Hz, 1H), 1.85 (dd, J=11.9, 5.9 Hz, 1H), 1.77-1.69 (m, 1H), 1.60 (s, 3H), 1.53 (s, 4H), 1.28 (s, 9H), 1.17 (d, J=17.0 Hz, 1H). ESI-MS m/z calc. 582.218, found 583.4 (M+1)⁺; Retention time: 2.21 minutes (LC method A).

### Step 7: (14S)-8-tert-Butyl-17-(5-chloropyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 208 (diastereomer 2)

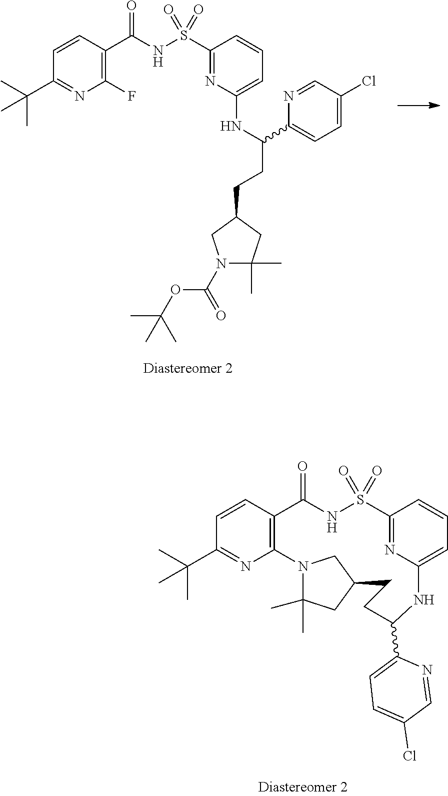 [SMILES: CC(C)(C)OC(=O)N1C[C@@H](CCC(Nc2cccc(S(=O)(=O)NC(=O)c3ccc(C(C)(C)C)nc3F)n2)c2ccc(Cl)cn2)CC1(C)C ; CC(C)(C)c1ccc2c(n1)N1C[C@@H](CCC(c3ccc(Cl)cn3)Nc3cccc(n3)S(=O)(=O)NC2=O)CC1(C)C]

tert-Butyl (4S)-4-[3-[[6-[(6-tert-butyl-2-fluoro-pyridine-3-carbonyl)sulfamoyl]-2-pyridyl]amino]-3-(5-chloro-2-pyridyl)propyl]-2,2-dimethyl-pyrrolidine-1-carboxylate (472 mg, 0.6712 mmol) (diastereomer 2) was dissolved in DCM (10 mL) and to the mixture was added TFA (2.5 mL, 32.45 mmol) and the reaction was stirred at room temperature. After 1 h, the mixture was evaporated to dryness, then diluted with diethyl ether (30 mL×2), and concentrated. The material was then placed on the high vacuum pump for 2 h to afford intermediate 6-tert-butyl-N-[[6-[[1-(5-chloro-2-pyridyl)-3-[(3S)-5,5-dimethylpyrrolidin-3-yl]propyl]amino]-2-pyridyl]sulfonyl]-2-fluoro-pyridine-3-carboxamide as an off-white solid. ESI-MS m/z calc. 602.22424, found 603.3 (M+1)⁺; Retention time: 1.4 minutes. The intermediate and K₂CO₃ (1.0 g, 7.236 mmol), 3 Å molecular sieves and NMP (12 mL) were combined in a vial, which was purged with nitrogen, capped, heated to 150° C. and stirred for 3 h, then 170° C. and stirred for 1 h. The mixture was diluted with ethyl acetate and water. The organic layer was extracted (2×) and was further washed with a 10% citric acid solution followed by brine. The organics were separated, dried over sodium sulfate, evaporated to a light brown oil. The residues was purified separately by reverse-phase preparative chromatography utilizing a Cis column and a 30-99% gradient over 15 min of acetonitrile in water (+5 mM HCl), to afford the diastereomer product as a white solid: (14S)-8-tert-butyl-17-(5-chloropyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 2 (134.2 mg, 34%). ¹H NMR (500 MHz, DMSO-d₆) δ 12.50 (s, 1H), 8.58 (d, J=2.5 Hz, 1H), 7.89 (dd, J=8.5, 2.5 Hz, 1H), 7.72 (d, J=9.1 Hz, 1H), 7.69-7.59 (m, 2H), 7.48 (d, J=8.4 Hz, 1H), 7.12 (d, J=7.2 Hz, 1H), 6.88 (d, J=8.4 Hz, 1H), 6.66 (d, J=8.0 Hz, 1H), 5.39-5.14 (m, 1H), 3.20 (dd, J=10.2, 6.8 Hz, 1H), 2.76 (t, J=10.5 Hz, 1H), 2.23 (s, 1H), 1.99-1.88 (m, 2H), 1.85 (dd, J=11.7, 5.2 Hz, 1H), 1.75 (d, J=13.8 Hz, 1H), 1.65 (s, 3H), 1.58 (t, J=12.3 Hz, 1H), 1.51 (s, 3H), 1.49-1.40 (m, 1H), 1.29 (s, 9H). ESI-MS m/z calc. 582.218, found 583.6 (M+1)⁺; Retention time: 2.15 minutes (LC method A).

The compounds in the following tables were prepared in a manner analogous to that described above, using 4-bromo-1-methyl pyrazole or phenyl lithium as reagents in step 1. For each prepared diastereomeric pair, the diastereomer 1 originated from from the first isomer to elute during the separation procedure in step 5. The diastereomer 2 originated from the second isomer to elute during the separation procedure in step 5.

| | | LCMS | | | |
|---|---|---|---|---|---|
| | | Retention | | | |
| Compound | | Time | Exact | | LCMS |
| Number | Structure | (min) | Mass | M + 1 | Method |
| Compound 295 (diastereomer 1) | 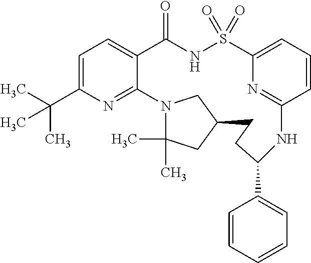 [SMILES: CC(C)(C)c1ccc2c(n1)N1C[C@@H](CC[C@@H](c3ccccc3)Nc3cccc(n3)S(=O)(=O)NC2=O)CC1(C)C] | 2.38 | 547.262 | 548.1 | LC method A |
| Compound 294 (diastereomer 2) | 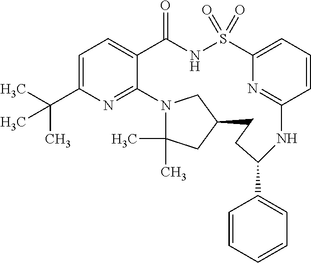 [SMILES: CC(C)(C)c1ccc2c(n1)N1C[C@@H](CC[C@@H](c3ccccc3)Nc3cccc(n3)S(=O)(=O)NC2=O)CC1(C)C] | 2.3 | 547.262 | 548.1 | LC method A |
| Compound 281 (diastereomer 1) | 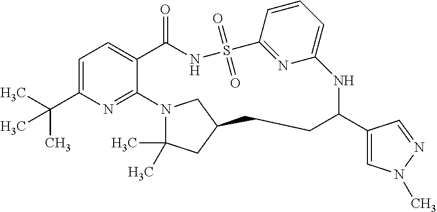 [SMILES: Cn1cc(C2CC[C@@H]3CN(c4nc(C(C)(C)C)ccc4C(=O)NS(=O)(=O)c4cccc(n4)N2)C(C)(C)C3)cn1] | 2.67 | 551.268 | 552.3 | LC method A |
| Compound 280 (diastereomer 2) | 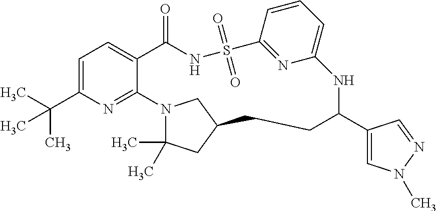 [SMILES: Cn1cc(C2CC[C@@H]3CN(c4nc(C(C)(C)C)ccc4C(=O)NS(=O)(=O)c4cccc(n4)N2)C(C)(C)C3)cn1] | 2.76 | 551.268 | 552.3 | LC method A |

| Compound | | |
|---|---|---|
| Number | Structure | NMR |
| Compound 295 (diastereomer 1) | 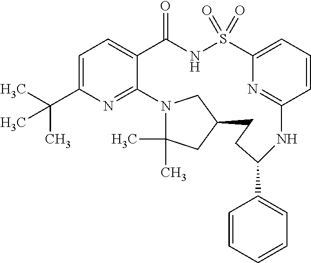 [SMILES: CC(C)(C)c1ccc2c(n1)N1C[C@@H](CC[C@@H](c3ccccc3)Nc3cccc(n3)S(=O)(=O)NC2=O)CC1(C)C] | ¹H NMR (500 MHz, DMSO-d₆) δ 12.28 (s, 1H), 7.65 (d, J = 8.1 Hz, 1H), 7.60 (t, J = 7.9 Hz, 1H), 7.51 (d, J = 7.7 Hz, 3H), 7.33 (t, J = 7.6 Hz, 2H), 7.25 (d, J = 7.6 Hz, 1H), 7.19 (d, J = 7.2 Hz, 1H), 6.77 (d, J = 8.5 Hz, 1H), 6.65 (d, J = 7.9 Hz, 1H), 4.87 (s, 1H), 3.15 (s, 2H), 2.39-2.21 (m, 2H), 1.97-1.79 (m, 2H), 1.76-1.67 (m, 1H), 1.62 (s, 3H), 1.54 (s, 3H), 1.50 (s, 1H), 1.29 (s, 9H), 1.16 (s, 1H). |
| Compound 294 (diastereomer 2) | 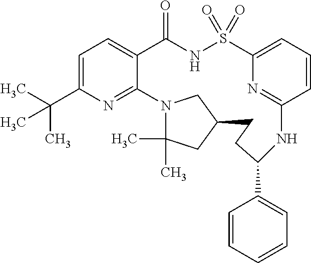 [SMILES: CC(C)(C)c1ccc2c(n1)N1C[C@@H](CC[C@@H](c3ccccc3)Nc3cccc(n3)S(=O)(=O)NC2=O)CC1(C)C] | ¹H NMR (500 MHz, DMSO-d₆) δ 12.51 (s, 1H), 7.64 (dt, J = 17.5, 8.7 Hz, 3H), 7.42 (d, J = 7.6 Hz, 2H), 7.30 (t, J = 7.5 Hz, 2H), 7.20 (t, J = 7.4 Hz, 1H), 7.07 (d, J = 7.3 Hz, 1H), 6.81 (d, J = 8.5 Hz, 1H), 6.67 (dd, J = 7.9, 2.1 Hz, 1H), 5.20 (t, J = 11.2 Hz, 1H), 3.25 (t, J = 8.7 Hz, 1H), 2.80 (t, J = 10.5 Hz, 1H), 2.27 (s, 1H), 1.94 (d, J = 5.2 Hz, 1H), 1.88-1.79 (m, 1H), 1.72 (d, J = 13.2 Hz, 2H), 1.66 (s, 3H), 1.58 (t, J = 12.3 Hz, 1H), 1.52 (s, 3H), 1.47 (d, J = 12.7 Hz, 1H), 1.29 (d, J = 23 Hz, 9H). |

### Example 14: Preparation of (14S)-8-tert-butyl-12,12-dimethyl-17-(propan-2-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 287 (diastereomer 1), and Compound 286 (diastereomer 2)

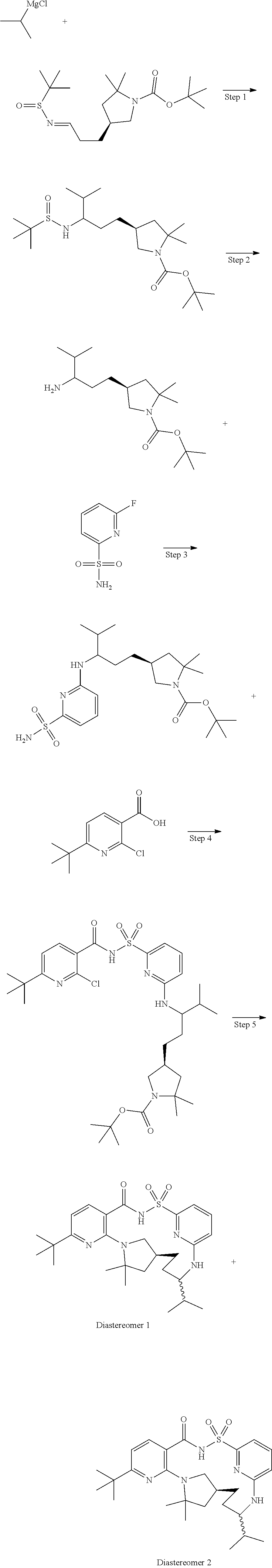

### Step 1: tert-Butyl (4S)-4-[3-(tert-butylsulfinylamino)-4-methyl-pentyl]-2,2-dimethyl-pyrrolidine-1-carboxylate

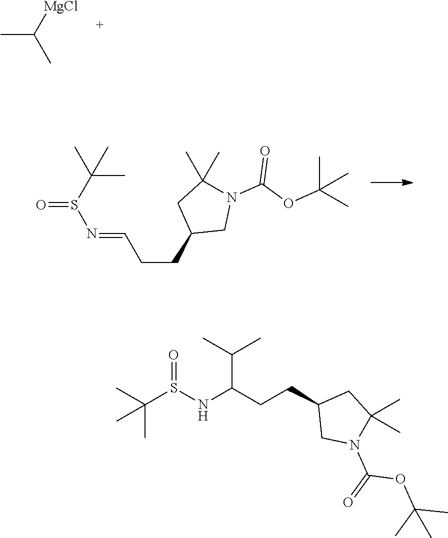

tert-Butyl (4S)-4-[(3E)-3-tert-butylsulfinyliminopropyl]-2,2-dimethyl-pyrrolidine-1-carboxylate (10 g, 26.50 mmol) was dissolved in THF (150 mL). The solution was cooled to −78° C. using a dry ice acetone bath under nitrogen balloon and stirred 5 min. Isopropyl magnesium chloride (1.3M in THF) (125 mL of 1.3 M, 162.50 mmol) was added dropwise. The mixture was allowed to warm up to ˜−35° C. and stirred for 1 hour. Saturated aqueous NH₄Cl (80 mL) was added. The mixture was warmed up to rt. EtOAc (200 mL) and water (100 mL) were added. The layers were separated and the aqueous layer was extracted once with EtOAc (200 mL). The combined organics was washed with brine, dried over anhydrous Na₂SO₄, filtered and concentrated to give crude tert-butyl(4S)-4-[3-(tert-butylsulfinylamino)-4-methyl-pentyl]-2,2-dimethyl-pyrrolidine-1-carboxylate (12.5 g, 100%). ESI-MS m/z calc. 402.2916, found 403.6 (M+1)⁺; Retention time: 4.02 minutes (LC method B).

### Step 2: tert-Butyl (4S)-4-(3-amino-4-methyl-pentyl)-2,2-dimethyl-pyrrolidine-1-carboxylate

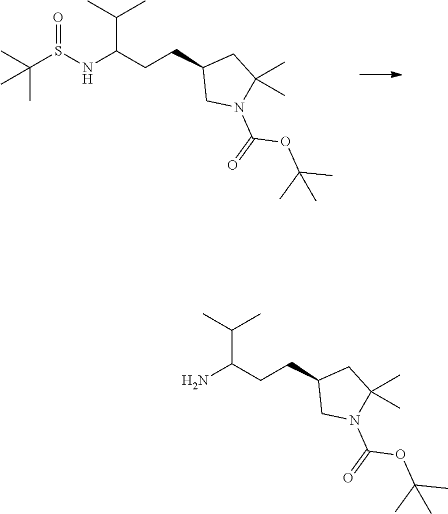 [SMILES: CC(C)C(CC[C@@H]1CN(C(=O)OC(C)(C)C)C(C)(C)C1)NS(=O)C(C)(C)C ; CC(C)C(N)CC[C@@H]1CN(C(=O)OC(C)(C)C)C(C)(C)C1]

tert-Butyl (4S)-4-[3-(tert-butylsulfinylamino)-4-methyl-pentyl]-2,2-dimethyl-pyrrolidine-1-carboxylate (12.5 g, 26.389 mmol) was dissolved in a mixed solvent of THF (180 mL) and water (36 mL). Molecular iodine (2 g, 7.864 mmol) was added in one portion. The mixture was stirred at 35° C. for 18 h. It was then cooled to rt and partitioned between EtOAc (300 mL) and Na₂S₂O₃ (50 g) in saturated aqueous sodium bicarbonate (300 mL). The layers were separated and the aqueous layer was extracted once with EtOAc (200 mL). The combined organic layer was concentrated. The residue was dissolved in 1M HCl (500 mL) and was extracted with diethyl ether (300 mL). The aqueous layer was basified by 2.5M NaOH and extracted with EtOAc (2×300 mL). The combined organic layer was washed with brine, dried over anhydrous Na₂SO₄, filtered and concentrated to give tert-butyl (4S)-4-(3-amino-4-methyl-pentyl)-2,2-dimethyl-pyrrolidine-1-carboxylate (6.8 g, 82%). ESI-MS m/z calc. 298.262, found 299.7 (M+1)⁺; Retention time: 2.69 minutes (LC method B).

### Step 3: tert-Butyl (4S)-2,2-dimethyl-4-[4-methyl-3-[(6-sulfamoyl-2-pyridyl)amino]pentyl]pyrrolidine-1-carboxylate

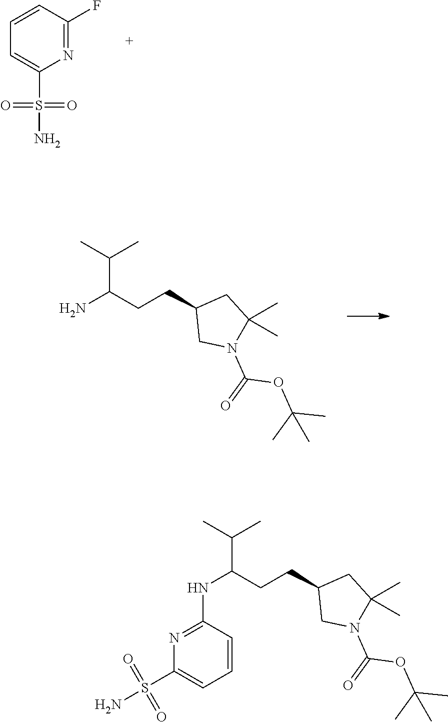 [SMILES: NS(=O)(=O)c1cccc(F)n1 ; CC(C)C(N)CC[C@@H]1CN(C(=O)OC(C)(C)C)C(C)(C)C1 ; CC(C)C(CC[C@@H]1CN(C(=O)OC(C)(C)C)C(C)(C)C1)Nc1cccc(S(N)(=O)=O)n1]

To a mixture of tert-butyl (4S)-4-(3-amino-4-methyl-pentyl)-2,2-dimethyl-pyrrolidine-1-carboxylate (6.8 g, 21.644 mmol) and 6-fluoropyridine-2-sulfonamide (7.5 g, 40.444 mmol) in DMSO (20 mL) was added DIEA (12 mL, 68.893 mmol). The mixture was stirred at 115° C. for 20 h. It was then cooled to rt and partitioned between EtOAc (300 mL) and saturated sodium bicarbonate (500 mL). The aqueous layer was extracted with more EtOAc (300 mL). The combined EtOAc solution was washed with brine (2×500 mL), dried over anhydrous Na₂SO₄, filtered and concentrated. The residue was purified by silica gel chromatography, using 0-70% EtOAc in hexanes to afford tert-butyl (4S)-2,2-dimethyl-4-[4-methyl-3-[(6-sulfamoyl-2-pyridyl)amino]pentyl]pyrrolidine-1-carboxylate (8.1 g, 79%) as a solid. ¹H NMR (500 MHz, DMSO-d₆) δ 7.48 (t, J=7.8, 7.8 Hz, 1H), 7.03 (s, 2H), 6.90 (d, J=7.1 Hz, 1H), 6.72 (d, J=9.1 Hz, 1H), 6.65 (d, J=8.3 Hz, 1H), 4.01-3.87 (m, 1H), 3.59-3.47 (m, 1H), 2.80-2.69 (m, 1H), 2.13-2.01 (m, 1H), 1.92-1.73 (m, 2H), 1.58-1.44 (m, 1H), 1.44-1.26 (m, 17H), 1.23 (d, J=2.9 Hz, 3H), 0.91-0.84 (m, 6H). ESI-MS m/z calc. 454.2614, found 455.2 (M+1)⁺; Retention time: 2.77 minutes (LC method H).

### Step 4: tert-Butyl (4S)-4-[3-[[6-[(6-tert-butyl-2-chloro-pyridine-3-carbonyl)sulfamoyl]-2-pyridyl]amino]-4-methyl-pentyl]-2,2-dimethyl-pyrrolidine-1-carboxylate

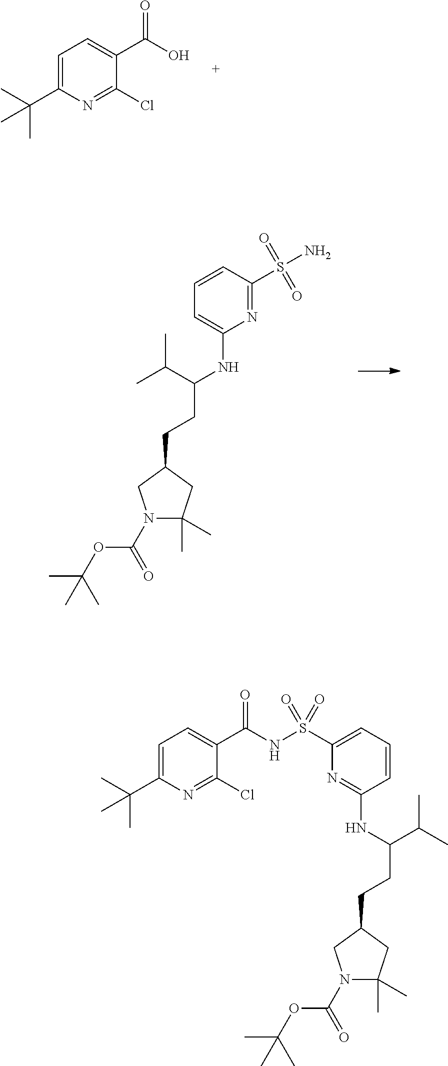 [SMILES: CC(C)(C)c1ccc(C(=O)O)c(Cl)n1 ; CC(C)C(CC[C@@H]1CN(C(=O)OC(C)(C)C)C(C)(C)C1)Nc1cccc(S(N)(=O)=O)n1 ; CC(C)C(CC[C@@H]1CN(C(=O)OC(C)(C)C)C(C)(C)C1)Nc1cccc(S(=O)(=O)NC(=O)c2ccc(C(C)(C)C)nc2Cl)n1]

To a solution of 6-tert-butyl-2-chloro-pyridine-3-carboxylic acid (175 mg, 0.819 mmol) in THF (6 mL) was added CDI (135 mg, 0.833 mmol) (recrystallized from THF) and the mixture was stirred at rt for 3 h. Then tert-butyl (4S)-2,2-dimethyl-4-[4-methyl-3-[(6-sulfamoyl-2-pyridyl)amino]pentyl]pyrrolidine-1-carboxylate (266 mg, 0.585 mmol) was added followed by DBU (300 μL, 2.01 mmol) and the resulting mixture was stirred for 16 h at rt. The reaction was diluted with ethyl acetate and washed with a saturated aqueous sodium bicarbonate solution. The organic layer was further washed with 10% citric acid solution followed by brine. The organics were separated, dried over sodium sulfate, evaporated and then purified on silica gel chromatography (80 gram column) using a gradient from 100% hexanes to 70% ethyl acetate in hexanes followed by a second silica gel column (24 gram column) using a gradient from 100% dichloromethane to 15% methanol in dichloromethane to afford tert-butyl (4S)-4-[3-[[6-[(6-tert-butyl-2-chloro-pyridine-3-carbonyl)sulfamoyl]-2-pyridyl]amino]-4-methyl-pentyl]-2,2-dimethyl-pyrrolidine-1-carboxylate as an off-white solid (123 mg, 32%). ESI-MS m/z calc. 649.30646, found 650.2 (M+1)⁺; Retention time: 1.99 minutes (LC method A).

### Step 5: (14S)-8-tert-Butyl-12,12-dimethyl-17-(propan-2-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 287 (diastereomer 1), and (14S)-8-tert-butyl-12,12-dimethyl-17-(propan-2-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 286 (diastereomer 2)

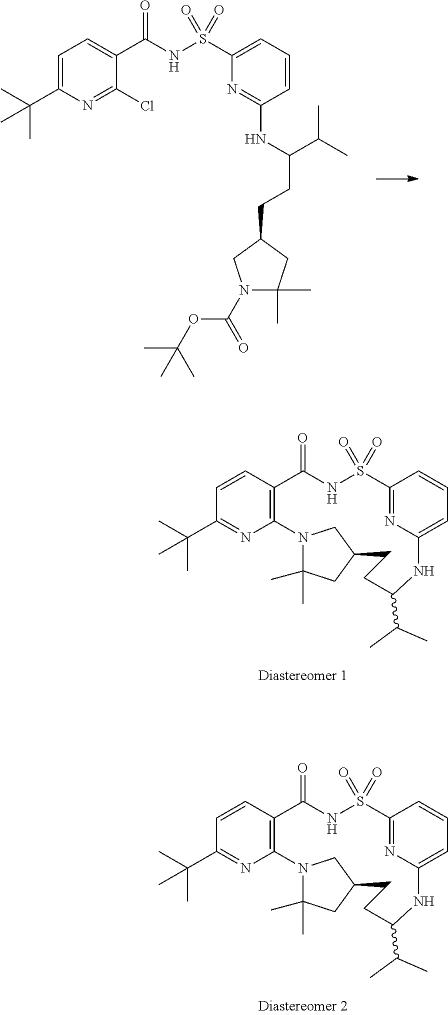

Stage 1: tert-Butyl (4S)-4-[3-[[6-[(6-tert-butyl-2-chloro-pyridine-3-carbonyl)sulfamoyl]-2-pyridyl]amino]-4-methyl-pentyl]-2,2-dimethyl-pyrrolidine-1-carboxylate (120 mg, 0.1845 mmol) was dissolved in DCM (3.0 mL) and to the mixture was added HCl (1.75 mL of 4 M in dioxane, 7.00 mmol); and the resulting mixture was stirred at room temperature. After 2 h, the mixture was evaporated to dryness, then diluted with diethyl ether (5 mL×2), and reconcentrated. The material was then placed on the high vacuum pump for 2 h to afford the intermediate 6-tert-butyl-2-chloro-N-[[6-[[1-[2-[(3S)-5,5-dimethylpyrrolidin-3-yl]ethyl]-2-methyl-propyl]amino]-2-pyridyl]sulfonyl]pyridine-3-carboxamide (hydrochloride salt) as an off-white solid. ESI-MS m/z calc. 549.254, found 550.2 (M+1)⁺; Retention time: 1.49 minutes. Retention time: 1.76 minutes (LC method A).

Stage 2: The material from stage 1 was combined with K₂CO₃ (300 mg, 2.171 mmol), 3 Å molecular sieves and DMSO (4 mL) in a vial. The resulting mixture was purged with nitrogen, capped, heated to 155° C. and stirred for 36 h. The mixture was cooled to rt, filtered and concentrated under a stream of nitrogen to give a residue which was purified by reverse-phase preparative chromatography utilizing a C₁₈ column and a 30-99% gradient over 15 min of acetonitrile in water (+5 mM HCl) to afford two products:

Diastereomer 1: Peak 1, more polar, off-white solid: (14S)-8-tert-Butyl-12,12-dimethyl-17-(propan-2-yl)-2λ⁶-thia-3,9,11,18,23-pentaazatetracyclo[17.3.1.111,14.05,10]tetracosa-1(23),5(10),6,8,19,21-hexaene-2,2,4-trione (11.85 mg, 25%). ¹H NMR (500 MHz, DMSO-d₆) δ 12.42 (s, 1H), 7.62 (d, J=7.9 Hz, 1H), 7.55 (t, J=7.9 Hz, 1H), 7.02 (d, J=7.2 Hz, 1H), 6.94 (d, J=9.1 Hz, 1H), 6.74 (d, J=8.5 Hz, 1H), 6.65 (d, J=8.0 Hz, 1H), 3.96 (s, 1H), 3.09 (d, J=9.7 Hz, 1H), 2.65 (d, J=10.5 Hz, 1H), 2.13-1.98 (m, 1H), 1.89-1.74 (m, 1H), 1.65 (d, J=5.7 Hz, 1H), 1.62 (s, 3H), 1.53 (d, J=13.0 Hz, 2H), 1.49 (s, 3H), 1.48-1.42 (m, 1H), 1.28 (s, 11H), 0.87 (t, J=7.2 Hz, 6H). ESI-MS m/z calc. 513.27734, found 514.2 (M+1)⁺; Retention time: 2.24 minutes (LC method A).

Diastereomer 2: Peak 2, less polar, off-white solid: (14S)-8-tert-Butyl-12,12-dimethyl-17-(propan-2-yl)-2λ⁶-thia-3,9,11,18,23-pentaazatetracyclo[17.3.1.111,14.05,10]tetracosa-1(23),5(10),6,8,19,21-hexaene-2,2,4-trione (7.86 mg, 17%). ¹H NMR (500 MHz, DMSO-d₆) δ 12.40 (s, 1H), 7.56 (t, J=7.9 Hz, 1H), 7.42 (d, J=7.9 Hz, 1H), 7.21-7.04 (m, 2H), 6.76 (d, J=8.6 Hz, 1H), 6.60 (d, J=7.9 Hz, 1H), 3.10 (s, 1H), 2.81 (s, 1H), 2.29-2.17 (m, 1H), 2.07-1.96 (m, 1H), 1.87-1.76 (m, 1H), 1.64-1.61 (m, 1H), 1.54 (d, J=15.1 Hz, 8H), 1.26 (s, 11H), 1.18 (s, 1H), 0.89-0.87 (m, 6H). ESI-MS m/z calc. 513.27734, found 514.2 (M+1)⁺; Retention time: 2.27 minutes (LC method A).

The following table contains a list of methods used for the preparation of various organomagnesium reagents:

| Starting Material | Preparation Method |
|---|---|
| chloro(me- | Mg (474 mg, 19.50 mmol) was weighed in a dry |
| thoxy)methane | flask. THF (5 mL) was added. Mercury (II) chloride |
| | (25 mg, 0.0916 mmol) was added. 2 drops of |
| | chloro(methoxy)methane were added and then |
| | chloro(methoxy)methane (1.6685 g, 1.65 mL, 19.77 |
| | mmol) was added. The mixture was stirred until |
| | activation was noticed (exothermic). The mixture |
| | was cooled to between −25° C. and −30° C. The |
| | remaining chloro(methoxy)methane was added |
| | dropwise over 10 min. The mixture was stirred |
| | under a nitrogen balloon for 1 h at the |
| | same temperature |
| 4- | Mg (338 mg, 13.907 mmol) was activated by heating |
| bromotetra- | with a tiny I₂ crystal under nitrogen until the iodine |
| hydropyran | vapor was visible. THF (15 mL) was added. A drop |
| | of 1,2 dibromoethane was added and the mixture was |
| | heated for a few minutes. A THF (5 mL) solution of |
| | 4-bromotetrahydropyran (1.45 g, 0.99 mL, 8.5228 |
| | mmol) was added dropwise over 30 min. The |
| | mixture was refluxed 3 h. It was then cooled in an |
| | dry ice acetone bath (−40° C.). |
| 2-bromo-1,3- | A solution of 2-bromo-1,3-dimethyl-benzene (4.5 g, |
| dimethyl- | 23.344 mmol) in tetrahydrofuran (9 mL) was added |
| benzene | dropwise to a slurry of magnesium (600 mg, 24.686 |
| | mmol) and iodine (20 mg, 0.0786 mmol) in |
| | tetrahydrofuran (5 mL). The reaction is exothermic: |
| | the rate of addition was adjusted to maintain the |
| | temperature below 60° C. during the course of the |
| | addition. The resulting mixture was refluxed under |
| | nitrogen for 2 h, then was cooled in a dry ice- |
| | methanol bath. |
| 3-bromo-2-iodo- | 3-Bromo-2-iodo-pyridine (8.26 g, 28.514 mmol) |
| pyridine | was dissolved in THF (72 mL) and the solution was |
| | cooled in a dry ice bath (−50° C.) under a nitrogen |
| | atmosphere. Isopropylmagnesium chloride lithium |
| | chloride (22 mL of 1.3M, 28.600 mmol) was added |
| | dropwise The mixture was stirred at this temperature |
| | for 1.5 h. |

The compounds in the following tables were prepared in a manner analogous to that described above using in step 1 organomagnesium reagents given in the table above.

For the preparation of Compound 235 and Compound 234, the organomagnesium reagent used in step 1 was commercially available cyclohexylmagnesium chloride and the separation of diastereomers was carried out at step 4.

Unless otherwise stated, for each prepared diastereomeric pair, the diastereomer 1 was the first isomer to elute during the separation procedure. The diastereomer 2 was the second isomer to elute.

The diastereomers in the following tables were separated by the following methods:

| Compound | |
|---|---|
| Number | Separation Method |
| Compound 277 | Preparative SFC on column phenomenex LUX-4 |
| (diastereomer | (250 × 21.2 mm), 5 μM, 40° C.; |
| 1) and | mobile phase: 34% MeOH, 66% CO₂, |
| Compound 276 | flow 70.0 mL/min, concentration: 30 mg/mL in |
| (diastereomer | methanol, injection volume (500 μL), pressure |
| 2) | 167 bar, wavelength: 210 nm. |
| Compound 269 | Preparative HPLC C₁₈ column and |
| (diastereomer | HPLC-MS method 20-80% gradient of acetonitrile |
| 1) and | in water + 5 mM HCl, over 30 minutes. |
| Compound 268 | |
| (diastereomer | |
| 2) | |
| Compound 243 | Preparative HPLC C₁₈ column and |
| (diastereomer | HPLC-MS method 30-99% gradient of acetonitrile |
| 1) and | in water + 5 mM HCl, over 15 minutes. |
| Compound 242 | |
| (diastereomer | |
| 2) | |
| Compound 54 | Silica gel chromatography (80 g column), |
| (diastereomer | using a gradient from 100% hexanes to 100% |
| 1) and | ethyl acetate. Diastereomer 1 is the less |
| Compound 53 | polar isomer (eluted first). Diastereomer 2 is |
| (diastereomer | the more polar isomer (eluted second). |
| 2) | |

| Compound | | |
|---|---|---|
| Number | Structure | NMR |
| Compound 277 (diastereomer 1) | 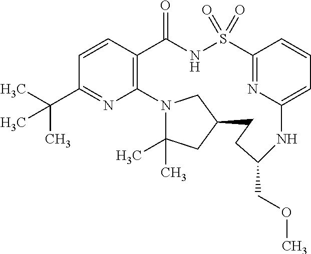 [SMILES: COC[C@@H]1CC[C@@H]2CN(c3nc(C(C)(C)C)ccc3C(=O)NS(=O)(=O)c3cccc(n3)N1)C(C)(C)C2] | ¹H NMR (500 MHz, DMSO-d₆) δ 12.54 (s, 1H), 7.58 (dd, J = 12.1, 7.8 Hz, 2H), 7.07 (d, J = 7.1 Hz, 1H), 6.99 (d, J = 9.1 Hz, 1H), 6.72 (d, J = 8.5 Hz, 1H), 6.63 (d, J = 7.9 Hz, 1H), 4.25 (s, 1H), 3.26 (s, 1H), 3.24 (s, 3H), 3.11 (dd, J = 9.3, 6.7 Hz, 1H), 3.02 (t, J = 8.7 Hz, 1H), 2.67 (t, J = 10.6 Hz, 1H), 2.07 (s, 1H), 1.80 (dd, J = 11.9, 5.3 Hz, 1H), 1.65 (s, 1H), 1.61 (s, 4H), 1.52 (d, J = 12.4 Hz, 1H), 1.48 (s, 3H), 1.37-1.28 (m, 1H), 1.27 (s, 10H). |
| Compound 276 (diastereomer 2) | 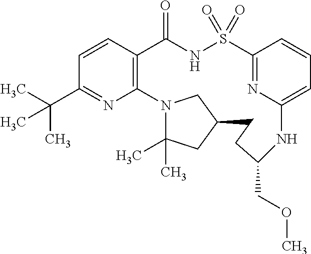 [SMILES: COC[C@@H]1CC[C@@H]2CN(c3nc(C(C)(C)C)ccc3C(=O)NS(=O)(=O)c3cccc(n3)N1)C(C)(C)C2] | ¹H NMR (500 MHz, DMSO-d₆) δ 12.41 (s, 1H), 7.57 (t, J = 7.9 Hz, 1H), 7.41 (d, J = 7.9 Hz, 1H), 7.26 (d, J = 6.8 Hz, 1H), 7.19 (d, J = 7.3 Hz, 1H), 6.75 (d, J = 8.5 Hz, 1H), 6.61 (d, J = 7.9 Hz, 1H), 3.60 (s, 2H), 3.38-3.32 (m, 3H), 3.13 (s, 1H), 2.86 (s, 1H), 2.25 (d, J = 14.4 Hz, 1H), 2.15-1.90 (m, 1H), 1.83 (dd, J = 12.1, 5.8 Hz, 1H), 1.65 (s, 1H), 1.57-1.46 (m, 8H), 1.26 (s, 10H), 1.19-1.12 (m, 1H). |
| Compound 269 (diastereomer 1) | 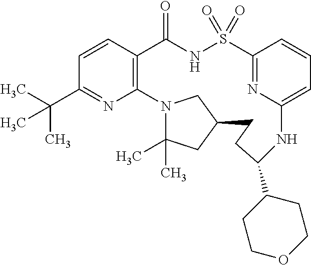 [SMILES: CC(C)(C)c1ccc2c(n1)N1C[C@@H](CC[C@@H](C3CCOCC3)Nc3cccc(n3)S(=O)(=O)NC2=O)CC1(C)C] | ¹H NMR (500 MHz, DMSO-d₆) δ 12.47 (s, 1H), 7.56 (t, J = 7.9 Hz, 1H), 7.41 (d, J = 7.8 Hz, 1H), 7.18 (t, J = 10.2 Hz, 2H), 6.76 (d, J = 8.5 Hz, 1H), 6.60 (d, J = 7.9 Hz, 1H), 3.92 (s, 4H), 3.87-3.81 (m, 2H), 3.21- 3.12 (m, 2H), 2.78 (s, 1H), 2.23 (s, 1H), 1.97 (s, 1H), 1.81 (dd, J = 11.8, 5.7 Hz, 1H), 1.64 (d, J = 7.8 Hz, 1H), 1.58 (d, J = 11.3 Hz, 2H), 1.53 (d, J = 11.0 Hz, 6H), 1.47 (s, 1H), 1.26 (s, 12H). |
| Compound 268 (diastereomer 2) | 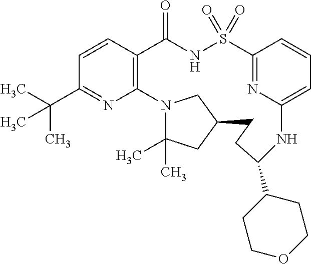 [SMILES: CC(C)(C)c1ccc2c(n1)N1C[C@@H](CC[C@@H](C3CCOCC3)Nc3cccc(n3)S(=O)(=O)NC2=O)CC1(C)C] | ¹H NMR (500 MHz, DMSO-d₆) δ 12.46 (s, 1H), 7.61 (d, J = 7.9 Hz, 1H), 7.55 (t, J = 7.8 Hz, 1H), 7.01 (d, J = 7.2 Hz, 1H), 6.96 (d, J = 9.0 Hz, 1H), 6.73 (d, J = 8.5 Hz, 1H), 6.64 (d, J = 7.9 Hz, 1H), 4.05 (s, 4H), 3.93 (s, 1H), 3.81 (dt, J = 10.3, 4.8 Hz, 2H), 3.26-3.11 (m, 2H), 3.05 (dd, J = 10.4, 7.0 Hz, 1H), 2.65 (t, J = 10.5 Hz, 1H), 2.05 (s, 1H), 1.79 (dd, J = 12.0, 5.3 Hz, 1H), 1.64 (s, 1H), 1.61 (s, 4H), 1.57- 1.50 (m, 2H), 1.47 (s, 4H), 1.42-1.35 (m, 1H), 1.27 (s, 9H). |
| Compound 235 (diastereomer 1) | 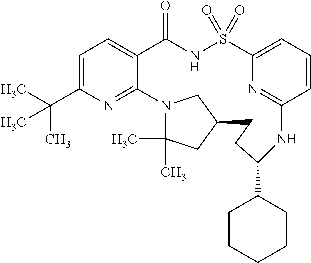 [SMILES: CC(C)(C)c1ccc2c(n1)N1C[C@@H](CC[C@@H](C3CCCCC3)Nc3cccc(n3)S(=O)(=O)NC2=O)CC1(C)C] | ¹H NMR (500 MHz, DMSO-d₆) δ 12.40 (s, 1H), 7.56 (dd, J = 8.5, 7.2 Hz, 1H), 7.42 (d, J = 7.9 Hz, 1H), 7.15 (d, J = 7.2 Hz, 1H), 7.07 (s, 1H), 6.75 (d, J = 8.5 Hz, 1H), 6.61 (d, J = 7.9 Hz, 1H), 3.09 (s, 1H), 2.84 (s, 1H), 2.24 (d, J = 10.2 Hz, 1H), 1.97 (s, 1H), 1.82 (dd, J = 11.9, 5.9 Hz, 1H), 1.78- 1.61 (m, 7H), 1.60 (s, 2H), 1.56 (s, 3H), 1.53 (s, 3H), 1.48 (s, 1H), 1.27 (s, 9H), 1.13 (dt, J = 19.9, 11.9 Hz, 4H), 0.97 (t, J = 11.0 Hz, 2H). |
| Compound 234 (diastereomer 2) | 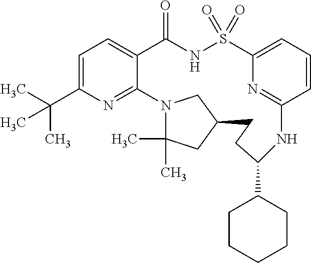 [SMILES: CC(C)(C)c1ccc2c(n1)N1C[C@@H](CC[C@@H](C3CCCCC3)Nc3cccc(n3)S(=O)(=O)NC2=O)CC1(C)C] | ¹H NMR (500 MHz, DMSO-d₆) δ 12.41 (s, 1H), 7.61 (d, J = 8.0 Hz, 1H), 7.54 (dd, J = 8.5, 7.2 Hz, 1H), 7.01 (d, J = 7.1 Hz, 1H), 6.89 (d, J = 9.1 Hz, 1H), 6.72 (d, 8.4 Hz, 1H), 6.64 (d, J = 8.0 Hz, 1H), 3.99- 3.88 (m, 1H), 3.06 (dd, 1H), 2.66 (t, J = 10.5 Hz, 1H), 2.09-1.99 (m, 1H), 1.80 (dd, J = 11.4, 5.2 Hz, 2H), 1.71-1.63 (m, 3H), 1.62 (s, 3H), 1.60-1.55 (m, 2H), 1.52 (d, J = 12.3 Hz, 2H), 1.48 (s, 3H), 1.28 (s, 9H), 1.26-1.21 (m, 2H), 1.18- 1.00 (m, 4H), 1.00-0.81 (m, 2H). |
| Compound 243 (diastereomer 1) | 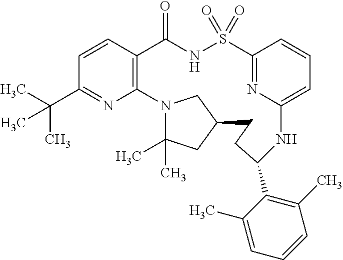 [SMILES: Cc1cccc(C)c1[C@@H]1CC[C@@H]2CN(c3nc(C(C)(C)C)ccc3C(=O)NS(=O)(=O)c3cccc(n3)N1)C(C)(C)C2] | ¹H NMR (500 MHz, DMSO-d₆) δ 12.50 (s, 1H), 7.64 (d, J = 8.0 Hz, 1H), 7.59 (dd, J = 8.5, 7.2 Hz, 1H), 7.48 (d, J = 8.3 Hz, 1H), 7.07 (d, J = 7.1 Hz, 1H), 6.98-6.85 (m, 4H), 6.67 (d, J = 8.0 Hz, 1H), 5.45 (dd, J = 13.2, 8.3 Hz, 1H), 3.22 (t, J = 8.6 Hz, 1H), 2.75 (t, J = 10.4 Hz, 1H), 2.47 (s, 6H), 2.23 (dd, J = 13.2, 6.3 Hz, 2H), 1.84 (dd, J = 11.8, 5.3 Hz, 1H), 1.76-1.66 (m, 1H), 1.61 (s, 3H), 1.57 (t, J = 12.3 Hz, 1H), 1.52 (s, 3H), 1.50-1.44 (m, 2H), 1.29 (s, 9H). |
| Compound 242 (diastereomer 2) | 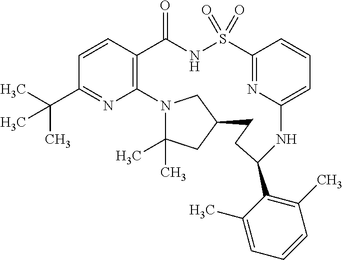 [SMILES: Cc1cccc(C)c1[C@H]1CC[C@@H]2CN(c3nc(C(C)(C)C)ccc3C(=O)NS(=O)(=O)c3cccc(n3)N1)C(C)(C)C2] | ¹H NMR (500 MHz, DMSO-d₆) δ 12.13 (s, 1H), 7.60 (dd, J = 8.5, 7.2 Hz, 1H), 7.55 (d, J = 8.0 Hz, 1H), 7.37 (d, J = 7.3 Hz, 1H), 7.19 (d, J = 7.1 Hz, 1H), 7.00 (q, J = 5.4 Hz, 3H), 6.88 (d, J = 8.5 Hz, 1H), 6.69 (d, J = 7.9 Hz, 1H), 5.56 (d, J = 7.0 Hz, 1H), 3.43 (t, J = 8.9 Hz, 1H), 3.00 (s, 1H), 2.49 (s, 6H), 2.40-2.27 (m, 1H), 2.04- 1.97 (m, 1H), 1.90 (s, 1H), 1.84 (dd, J = 11.9, 6.4 Hz, 1H), 1.72-1.67 (m, 1H), 1.65 (s, 4H), 1.51 (s, 3H), 1.42 (d, J = 14.7 Hz, 1H), 1.28 (s, 9H). |
| Compound 54 (diastereomer 1) | 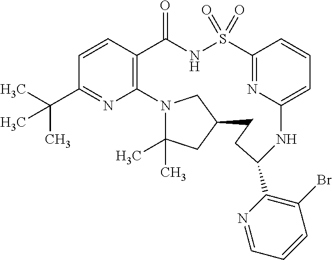 [SMILES: CC(C)(C)c1ccc2c(n1)N1C[C@@H](CC[C@@H](c3ncccc3Br)Nc3cccc(n3)S(=O)(=O)NC2=O)CC1(C)C] | ¹H NMR (400 MHz, DMSO-d₆) δ 12.16 (s, 1H), 8.64 (d, J = 4.6 Hz, 1H), 8.10 (dd, J = 8.1, 1.5 Hz, 1H), 7.61 (t, J = 7.9 Hz, 1H), 7.50 (d, J = 9.4 Hz, 2H), 7.32 (dd, J = 8.1, 4.6 Hz, 1H), 7.21 (d, J = 7.2 Hz, 1H), 6.90 (d, J = 8.2 Hz, 1H), 6.65 (d, J = 8.0 Hz, 1H), 5.77-5.63 (m, 1H), 3.53 (s, 1H), 3.03 (s, 1H), 2.33 (s, 1H), 2.18 (s, 1H), 1.81 (dd, J = 11.9, 6.0 Hz, 1H), 1.75 (s, 1H), 1.71 (s, 1H), 1.68-1.60 (m, 4H), 1.50 (s, 3H), 1.28 (s, 9H), 1.24 (s, 1H). |
| Compound 53 (diastereomer 2) | 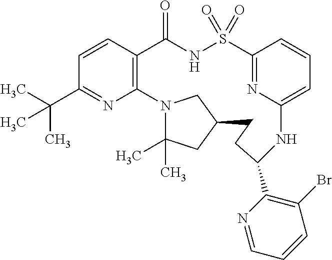 [SMILES: CC(C)(C)c1ccc2c(n1)N1C[C@@H](CC[C@@H](c3ncccc3Br)Nc3cccc(n3)S(=O)(=O)NC2=O)CC1(C)C] | ¹H NMR (400 MHz, DMSO-d₆) δ 12.37 (s, 1H), 8.60-8.47 (m, 1H), 8.02 (dd, J = 8.1, 1.5 Hz, 1H), 7.73 (d, J = 9.5 Hz, 1H), 7.64 (d, J = 8.0 Hz, 1H), 7.59 (t, J = 7.8 Hz, 1H), 7.24 (dd, J = 8.0, 4.6 Hz, 1H), 7.07 (d, J = 7.2 Hz, 1H), 6.89 (d, J = 8.5 Hz, 1H), 6.67 (d, J = 8.0 Hz, 1H), 5.80 (t, J = 10.7 Hz, 1H), 3.37 (t, J = 8.6 Hz, 1H), 2.85 (t, J = 10.4 Hz, 1H), 2.23 (s, 1H), 1.97- 1.80 (m, 2H), 1.74 (dd, 14.0, 5.7 Hz, 1H), 1.65 (s, 1H), 1.62 (s, 3H), 1.55 (d, J = 11.1 Hz, 1H), 1.52 (s, 3H), 1.47 (d, J = 11.3 Hz, 1H), 1.29 (s, 9H). |

| | LCMS | | | |
|---|---|---|---|---|
| | Retention | | | |
| Compound | Time | Exact | | LCMS |
| Number | (min) | Mass | M + 1 | Method |
| Compound 277 | 2.02 | 515.257 | 516.2 | LC method A |
| (diastereomer 1) | | | | |
| Compound 276 | 2.00 | 515.257 | 516.2 | LC method A |
| (diastereomer 2) | | | | |
| Compound 269 | 1.99 | 555.288 | 556.3 | LC method A |
| (diastereomer 1) | | | | |
| Compound 268 | 2.06 | 555.288 | 556.3 | LC method A |
| (diastereomer 2) | | | | |
| Compound 235 | 2.49 | 553.309 | 554.6 | LC method A |
| (diastereomer 1) | | | | |
| Compound 234 | 2.41 | 553.309 | 554.6 | LC method A |
| (diastereomer 2) | | | | |
| Compound 243 | 2.29 | 575.293 | 576.2 | LC method A |
| (diastereomer 1) | | | | |
| Compound 242 | 2.51 | 575.293 | 576.2 | LC method A |
| (diastereomer 2) | | | | |
| Compound 54 | 2.14 | 626.167 | 627.2 | LC method A |
| (diastereomer 1) | | | | |
| Compound 53 | 2.12 | 626.167 | 627.2 | LC method A |
| (diastereomer 2) | | | | |

### Example 15: Preparation of (14S)-8-tert-butyl-12,12-dimethyl-17-(pyrimidin-5-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 298 (diastereomer 1), and Compound 297 (diastereomer 2)

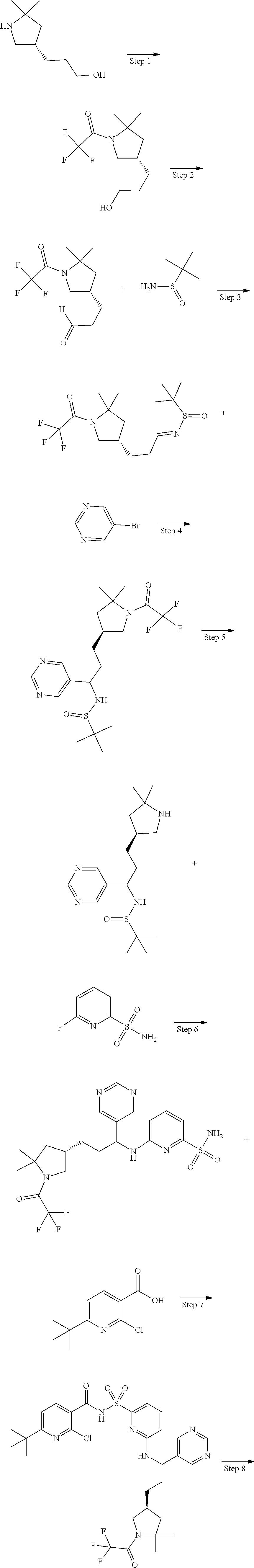

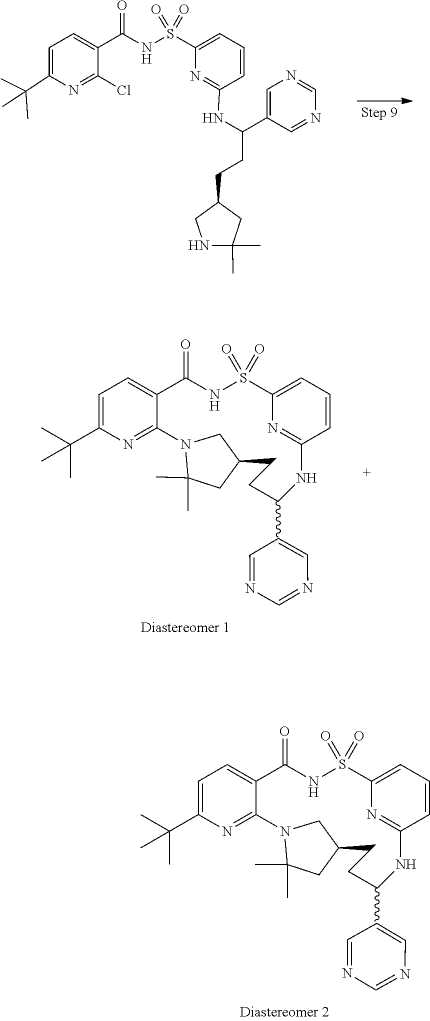 [SMILES: CC1(C)C[C@H](CCC(Nc2cccc(S(=O)(=O)NC(=O)c3ccc(C(C)(C)C)nc3Cl)n2)c2cncnc2)CN1 ; CC(C)(C)c1ccc2c(n1)N1C[C@@H](CCC(c3cncnc3)Nc3cccc(n3)S(=O)(=O)NC2=O)CC1(C)C ; CC(C)(C)c1ccc2c(n1)N1C[C@@H](CCC(c3cncnc3)Nc3cccc(n3)S(=O)(=O)NC2=O)CC1(C)C]

### Step 1: 2,2,2-Trifluoro-1-[(4S)-4-(3-hydroxypropyl)-2,2-dimethyl-pyrrolidin-1-yl]ethenone

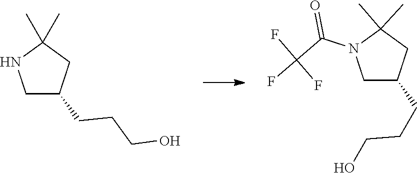 [SMILES: CC1(C)C[C@H](CCCO)CN1 ; CC1(C)C[C@H](CCCO)CN1C(=O)C(F)(F)F]

3-[(3S)-5,5-Dimethylpyrrolidin-3-yl]propan-1-01 (8.3 g, 50.142 mmol) was dissolved in DCM (100 mL). NEt₃ (14 mL, 100.44 mmol) was added and the mixture was stirred under a nitrogen balloon in ice water bath for 5 min. Trifluoroacetic anhydride (11 mL, 78.036 mmol) was added via syringe dropwise over 5 min. The mixture was stirred at rt for 2 h. It was then concentrated and the residue was taken a mixture of solvent MeOH/THF (20 mL each). A LiOH (1 g, 41.757 mmol) solution in water (20 mL) was added. The mixture was stirred at rt for 2 h. The mixture was concentrated and then partitioned between water and DCM (50 mL each). The layers were separated and the aqueous layer was further extracted with DCM (50 mL×2). The combined organics were dried over anhydrous MgSO₄, filtered and concentrated in vacuo to afford 2,2,2-trifluoro-1-[(4S)-4-(3-hydroxypropyl)-2,2-dimethyl-pyrrolidin-1-yl]ethanone (10.3 g, 77%). ESI-MS m/z calc. 253.129, found 254.3 (M+1)⁺; Retention time: 2.41 minutes (LC method B).

### Step 2: 3-[(3S)-5,5-Dimethyl-1-(2,2,2-trifluoroacetyl)pyrrolidin-3-yl]propanal

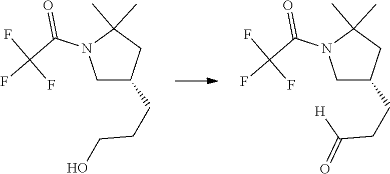 [SMILES: CC1(C)C[C@H](CCCO)CN1C(=O)C(F)(F)F ; CC1(C)C[C@H](CCC=O)CN1C(=O)C(F)(F)F]

2,2,2-Trifluoro-1-[(4S)-4-(3-hydroxypropyl)-2,2-dimethyl-pyrrolidin-1-yl]ethanone (7 g, 26.26 mmol) was dissolved in DCM (100 mL). The solution was cooled in ice water bath. Dess-Martin periodinane (12.895 g, 28.88 mmol) was added in small portions over 1 min. The mixture was stirred under nitrogen (balloon) while the ice bath was removed. After 2 h, a solution of NaS₂O₃ (˜10 g) in saturated aqueous sodium bicarbonate (50 mL) was added. The mixture was stirred for 30 min. The layers were separated. The DCM layer was further washed with sodium bicarbonate (saturated aqueous 50 mL×2), dried over anhydrous Na₂SO₄, filtered and concentrated. The oil residue was purified by silica gel chromatography (Rf: 0.48 3/1 hexanes/EtOAc), using 0-50% EtOAc in hexanes to afford 3-[(3S)-5,5-dimethyl-1-(2,2,2-trifluoroacetyl)pyrrolidin-3-yl]propanal (5.58 g, 80%). ESI-MS m/z calc. 251.1133, found 252.5 (M+1)⁺; Retention time: 3.54 minutes (LC method B).

### Step 3: N-[3-[(3S)-5,5-Dimethyl-1-(2,2,2-trifluoroacetyl)pyrrolidin-3-yl]propylidene]-2-methyl-propane-2-sulfinamide

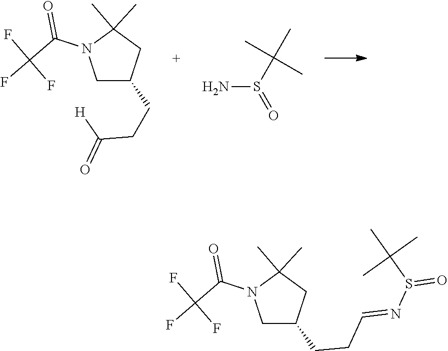

3-[(3S)-5,5-Dimethyl-1-(2,2,2-trifluoroacetyl)pyrrolidin-3-yl]propanal (7.5 g, 28.36 mmol) was dissolved in DCM (70 mL) at rt. 2-Methylpropane-2-sulfinamide (3.45 g, 27.896 mmol) was added, followed by magnesium sulfate (18 g, 148.79 mmol) and pyridinium p-toluenesulfonate (358 mg, 1.40 mmol). The mixture was stirred under nitrogen balloon for 18 h. It was then filtered through a pad of celite, which was washed with DCM. The combined filtrate was concentrated and the residue was purified by silica gel chromatography, using 5-60% EtOAc in hexanes to afford N-[3-[(3S)-5,5-dimethyl-1-(2,2,2-trifluoroacetyl)pyrrolidin-3-yl]propylidene]-2-methyl-propane-2-sulfinamide (9.8 g, 93%). ESI-MS m/z calc. 354.1589, found 355.5 (M+1)⁺; Retention time: 3.12 minutes (LC method B).

### Step 4: N-[3-[(3S)-5,5-Dimethyl-1-(2,2,2-trifluoroacetyl)pyrrolidin-3-yl]-1-pyrimidin-5-yl-propyl]-2-methyl-propane-2-sulfinamide

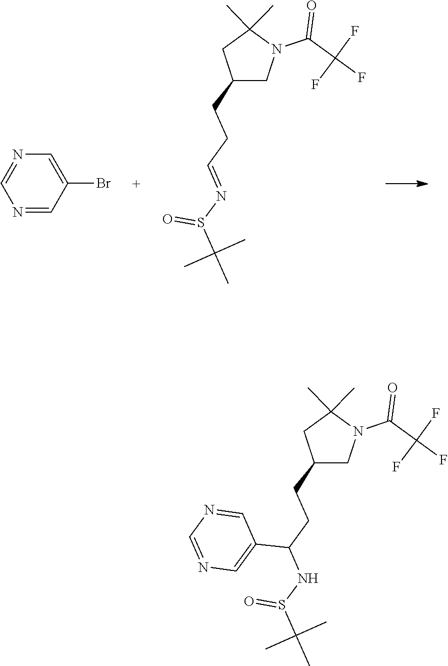 [SMILES: Brc1cncnc1 ; CC1(C)C[C@H](CC/C=N/S(=O)C(C)(C)C)CN1C(=O)C(F)(F)F ; CC1(C)C[C@H](CCC(NS(=O)C(C)(C)C)c2cncnc2)CN1C(=O)C(F)(F)F]

5-Bromopyrimidine (3.2 g, 19.725 mmol) was dissolved in THF (90 mL). The mixture was cooled in an ethanol liquid nitrogen bath until the bath temperature was <−100° C. and the solution was stirred for 10 min. n-BuLi (8 mL of 2.5 M in hexanes, 20.000 mmol) was added dropwise quickly along the inner wall of the reaction flask. The stirring stopped after several minutes. The mixture was allowed to stay unstirred in the cold bath for 20 min, then (NE)-N-[3-[(3S)-5,5-dimethyl-1-(2,2,2-trifluoroacetyl)pyrrolidin-3-yl]propylidene]-2-methyl-propane-2-sulfinamide (2.4 g, 6.4328 mmol) in THF (10 mL) was added along the inner wall of the flask over 3 min. The mixture was allowed to warm up in the same bath, and stirring started again when the bath temperature reached ˜−85° C. The reaction was continued below −70° C. for 15 min. Saturated NH₄Cl (20 mL) was added all at once. The mixture was allowed to warm up to rt and concentrated. The residue was partitioned between water and EtOAc (30 mL each). The aqueous layer was washed once with EtOAc. The EtOAc mixture was concentrated and purified by a short silica gel column, using 100% EtOAc as the eluant, to afford N-[3-[(3S)-5,5-dimethyl-1-(2,2,2-trifluoroacetyl)pyrrolidin-3-yl]-1-pyrimidin-5-yl-propyl]-2-methyl-propane-2-sulfinamide (1 g, 34%). ESI-MS m/z calc. 434.1963, found 435.6 (M+1)⁺; Retention time: 2.61 minutes (LC method B).

### Step 5: 1-[(4S)-4-(3-Amino-3-pyrimidin-5-yl-propyl)-2,2-dimethyl-pyrrolidin-1-yl]-2,2,2-trifluoro-ethanone

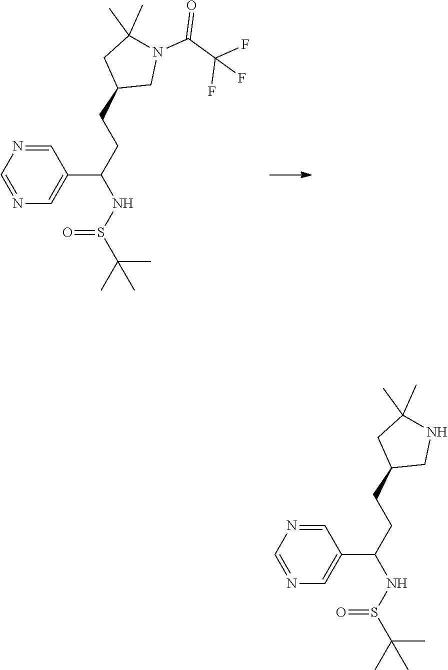 [SMILES: CC1(C)C[C@H](CCC(NS(=O)C(C)(C)C)c2cncnc2)CN1C(=O)C(F)(F)F ; CC1(C)C[C@H](CCC(NS(=O)C(C)(C)C)c2cncnc2)CN1]

N-[3-[(3S)-5,5-Dimethyl-1-(2,2,2-trifluoroacetyl)pyrrolidin-3-yl]-1-pyrimidin-5-yl-propyl]-2-methyl-propane-2-sulfinamide (1.2 g, 2.6236 mmol) was dissolved in MeOH (10 mL). An HCl (4 mL of 4 M, 16.000 mmol) solution in dioxane was added. The mixture was stirred at rt for 1 h. It was the concentrated and the residue was partitioned between DCM and aqueous sodium bicarbonate (20 mL each). The aqueous layer was extracted once (20 mL) with DCM. The combined DCM solution was washed with brine, dried over anhydrous Na₂SO₄, filtered and concentrated to afford 1-[(4S)-4-(3-amino-3-pyrimidin-5-yl-propyl)-2,2-dimethyl-pyrrolidin-1-yl]-2,2,2-trifluoro-ethanone (900 mg, 99%). ESI-MS m/z calc. 330.1667, found 331.4 (M+1)⁺; Retention time: 1.91 minutes (LC method B).

### Step 6: 6-[[3-[(3S)-5,5-Dimethyl-1-(2,2,2-trifluoroacetyl)pyrrolidin-3-yl]-1-pyrimidin-5-yl-propyl]amino]pyridine-2-sulfonamide

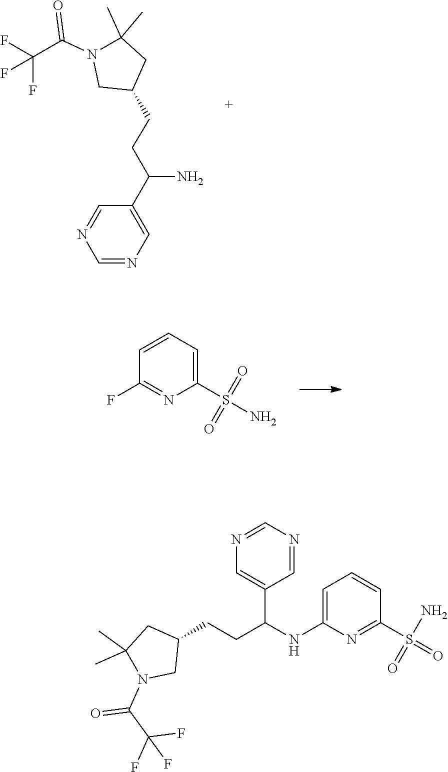 [SMILES: CC1(C)C[C@H](CCC(N)c2cncnc2)CN1C(=O)C(F)(F)F ; NS(=O)(=O)c1cccc(F)n1 ; CC1(C)C[C@H](CCC(Nc2cccc(S(N)(=O)=O)n2)c2cncnc2)CN1C(=O)C(F)(F)F]

1-[(4S)-4-(3-Amino-3-pyrimidin-5-yl-propyl)-2,2-dimethyl-pyrrolidin-1-yl]-2,2,2-trifluoro-ethanone (900 mg, 2.588 mmol) was dissolved in DMSO (2 mL). 6-Fluoropyridine-2-sulfonamide (768 mg, 4.359 mmol) and Na₂CO₃ (868 mg, 8.190 mmol) were added. The mixture was heated in a 110° C. oil bath under a nitrogen balloon for 18 h. It was then cooled to rt and partitioned between EtOAc and water (20 mL each). The organic layer was washed with water (10 mL×2), brine, dried over anhydrous Na₂SO₄, filtered and concentrated. The residue was purified by silica gel chromatography, using 5-80% EtOAc/hexanes to afford 6-[[3-[(3S)-5,5-dimethyl-1-(2,2,2-trifluoroacetyl)pyrrolidin-3-yl]-1-pyrimidin-5-yl-propyl]amino]pyridine-2-sulfonamide (0.504 g, 38%). ¹H NMR (500 MHz, DMSO-d₆) δ 9.03 (s, 1H), 8.89 (s, 2H), 7.65 (d, J=8.2 Hz, 1H), 7.54 (dd, J 8.4,7.2 Hz, 1H), 7.11 (s, 2H), 6.98 (d, J=7.2 Hz, 1H), 6.66 (d, J=8.4 Hz, 1H), 5.18-5.03 (m, 1H), 3.87-3.76 (m, 1H), 3.15 (q, J=11.3 Hz, 1H), 2.34-2.21 (m, 1H), 2.05-1.76 (m, 3H), 1.59-1.48 (m, 1H), 1.47-1.42 (m, 4H), 1.34 (s, 3H), 1.33-1.25 (m, 1H). ESI-MS m/z calc. 486.1661, found 487.3 (M+1)⁺; Retention time: 1.9 minutes (LC method H). Rf: ˜0.4, in 100% EtOAc.

### Step 7: 6-tert-Butyl-2-chloro-N-[[6-[[3-[(3S)-5,5-dimethyl-1-(2,2,2-trifluoroacetyl)pyrrolidin-3-yl]-1-pyrimidin-5-yl-propyl]amino]-2-pyridyl]sulfonyl]pyridine-3-carboxamide

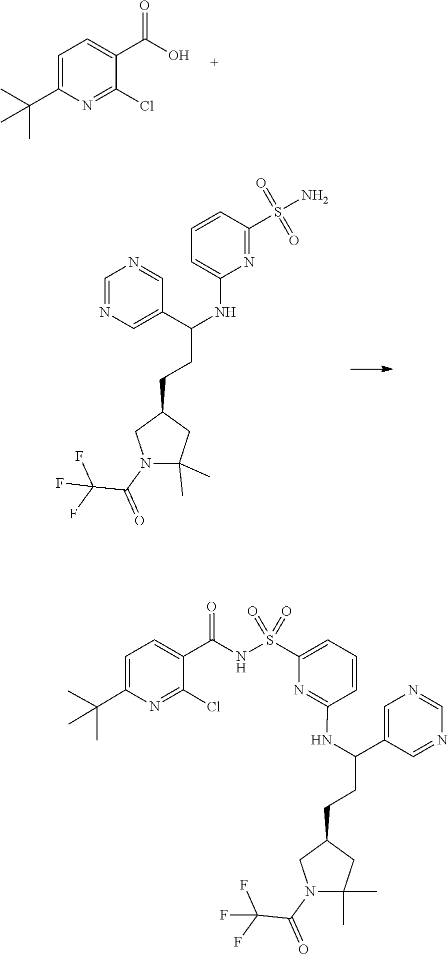 [SMILES: CC(C)(C)c1ccc(C(=O)O)c(Cl)n1 ; CC1(C)C[C@H](CCC(Nc2cccc(S(N)(=O)=O)n2)c2cncnc2)CN1C(=O)C(F)(F)F ; CC(C)(C)c1ccc(C(=O)NS(=O)(=O)c2cccc(NC(CC[C@@H]3CN(C(=O)C(F)(F)F)C(C)(C)C3)c3cncnc3)n2)c(Cl)n1]

To a solution of 6-tert-butyl-2-chloro-pyridine-3-carboxylic acid (255 mg, 1.193 mmol) in THF (12 mL) was added CDI (199 mg, 1.227 mmol) (recrystallized from THF) and the mixture was stirred at rt for 2 h then 6-[[3-[(3S)-5,5-dimethyl-1-(2,2,2-trifluoroacetyl)pyrrolidin-3-yl]-1-pyrimidin-5-yl-propyl]amino]pyridine-2-sulfonamide (350 mg, 0.719 mmol) was added followed by DBU (350 μL, 2.340 mmol) and the resulting mixture was stirred for 16 h at rt. The reaction was diluted with ethyl acetate and washed with a saturated aqueous sodium bicarbonate solution, and then the organic phased was washed with brine. The organics were separated, dried over sodium sulfate, evaporated and then purified on silica gel chromatography (80 gram column) using a gradient from 100% hexanes to 100% ethyl acetate followed by a second silica gel column (40 gram column) using a gradient from 100% dichloromethane to 15% methanol in dichloromethane to afford 6-tert-butyl-2-chloro-N-[[6-[[3-[(3S)-5,5-dimethyl-1-(2,2,2-trifluoroacetyl)pyrrolidin-3-yl]-1-pyrimidin-5-yl-propyl]amino]-2-pyridyl]sulfonyl]pyridine-3-carboxamide as an off-white solid (376 mg, 77%). ESI-MS m/z calc. 681.2112, found 682.2 (M+1)⁺; Retention time: 1.83 minutes (LC method A).

### Step 8: 6-tert-Butyl-2-chloro-N-[[6-[[3-[(3S)-5,5-dimethylpyrrolidin-3-yl]-1-pyrimidin-5-yl-propyl]amino]-2-pyridyl]sulfonyl]pyridine-3-carboxamide

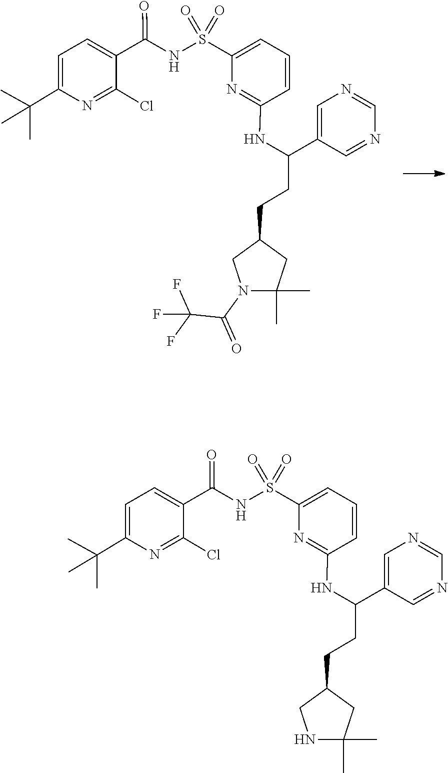 [SMILES: CC(C)(C)c1ccc(C(=O)NS(=O)(=O)c2cccc(NC(CC[C@@H]3CN(C(=O)C(F)(F)F)C(C)(C)C3)c3cncnc3)n2)c(Cl)n1 ; CC1(C)C[C@H](CCC(Nc2cccc(S(=O)(=O)NC(=O)c3ccc(C(C)(C)C)nc3Cl)n2)c2cncnc2)CN1]

A mixture of 6-tert-butyl-2-chloro-N-[[6-[[3-[(3S)-5,5-dimethyl-1-(2,2,2-trifluoroacetyl)pyrrolidin-3-yl]-1-pyrimidin-5-yl-propyl]amino]-2-pyridyl]sulfonyl]pyridine-3-carboxamide (376 mg, 0.551 mmol) and K₂CO₃ (800 mg, 5.788 mmol) in methanol (17 mL):water (7 mL) was heated under reflux and nitrogen atmosphere at 90° C. for 4 h. The mixture was cooled to rt, concentrated in vacuo and the residue was partitioned between water and EtOAc. The mixture was acidified slowly with 1 N HCl until pH=4. Then the mixture was further diluted with EtOAc, the organic layer was separated and the aqueous layer was re-extracted with ethyl acetate. The combined organic layers were washed with brine, dried over sodium sulfate and was evaporated to dryness to afford an off-white solid 6-tert-butyl-2-chloro-N-[[6-[[3-[(3S)-5,5-dimethylpyrrolidin-3-yl]-1-pyrimidin-5-yl-propyl]amino]-2-pyridyl]sulfonyl]pyridine-3-carboxamide (hydrochloride salt) (201 mg, 59%). ESI-MS m/z calc. 585.2289, found 586.2 (M+1)⁺; Retention time: 1.23 minutes (LC method A).

### Step 9: (14S)-8-tert-Butyl-12,12-dimethyl-17-(pyrimidin-5-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 298 (diastereomer 1), and (14S)-8-tert-butyl-12,12-dimethyl-17-(pyrimidin-5-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 297 (diastereomer 2)

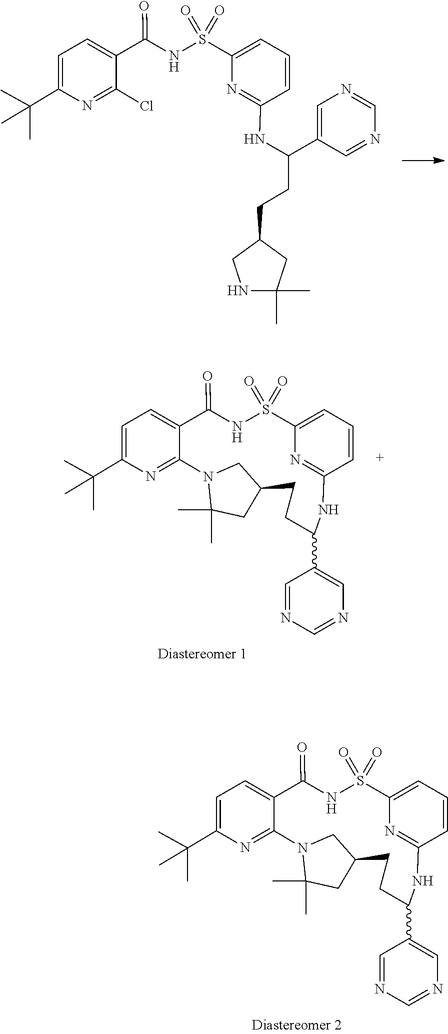 [SMILES: CC1(C)C[C@H](CCC(Nc2cccc(S(=O)(=O)NC(=O)c3ccc(C(C)(C)C)nc3Cl)n2)c2cncnc2)CN1 ; CC(C)(C)c1ccc2c(n1)N1C[C@@H](CCC(c3cncnc3)Nc3cccc(n3)S(=O)(=O)NC2=O)CC1(C)C ; CC(C)(C)c1ccc2c(n1)N1C[C@@H](CCC(c3cncnc3)Nc3cccc(n3)S(=O)(=O)NC2=O)CC1(C)C]

Dissolved 6-tert-butyl-2-chloro-N-[[6-[[3-[(3S)-5,5-dimethylpyrrolidin-3-yl]-1-pyrimidin-5-yl-propyl]amino]-2-pyridyl]sulfonyl]pyridine-3-carboxamide (hydrochloride salt) (181.1 mg, 0.2908 mmol), K₂CO₃ (400 mg, 2.894 mmol), 3 Å molecular sieves and DMSO (4.5 mL) in a 20 mL microwave vial. The resulting mixture was purged with nitrogen, capped, heated to 165° C. and stirred for 40 h. The mixture was cooled to ambient temperature and was filtered and concentrated under a stream of nitrogen to give a residue. This mixture was purified by reverse-phase preparative chromatography utilizing a C₁₈ column a 20-80% gradient over 30 min of acetonitrile in water (+5 mM HCl) to afford two product peaks, separated diastereomers:

Diastereomer 1: more polar, off-white solid: (14S)-8-tert-Butyl-12,12-dimethyl-17-(pyrimidin-5-yl)-2λ⁶-thia-3,9,11,18,23-pentaazatetracyclo[17.3.1.111,14.05,10]tetracosa-1(22),5,7,9,19(23),20-hexaene-2,2,4-trione (60 mg, 38%). ESI-MS m/z calc. 549.2522, found 550.2 (M+1)⁺; 1.82 minutes (LC method A).

Diastereomer 2: less polar, off-white solid: (14S)-8-tert-Butyl-12,12-dimethyl-17-(pyrimidin-5-yl)-2λ⁶-thia-3,9,11,18,23-pentaazatetracyclo[17.3.1.111,14.05,10]tetracosa-1(22),5,7,9,19(23),20-hexaene-2,2,4-trione (hydrochloride salt) (16.16 mg, 18%). ¹H NMR (500 MHz, DMSO-d₆) δ 12.53 (s, 1H), 9.12 (s, 1H), 8.96 (s, 2H), 8.03 (s, 1H), 7.69-7.60 (m, 2H), 7.11 (dd, J=7.2, 2.4 Hz, 1H), 6.85 (d, J=8.6 Hz, 1H), 6.65 (dd, J=8.0, 2.4 Hz, 1H), 5.24 (d, J=11.3 Hz, 1H), 3.24 (t, J=8.6 Hz, 1H), 2.75 (t, J=10.3 Hz, 1H), 2.28 (s, 1H), 2.14-2.06 (m, 1H), 1.82 (d, J=7.3 Hz, 1H), 1.78-1.69 (m, 2H), 1.63 (s, 3H), 1.55 (t, J=12.3 Hz, 1H), 1.49 (d, J=2.7 Hz, 3H), 1.48-1.36 (m, 1H), 1.26 (d, J=2.6 Hz, 9H). ESI-MS m/z calc. 549.2522, found 550.2 (M+1)⁺; Retention time: 1.89 minutes (LC method A).

### Example 16: Preparation of 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]benzoic acid, Compound 291 (diastereomer 1), and Compound 290 (diastereomer 2)

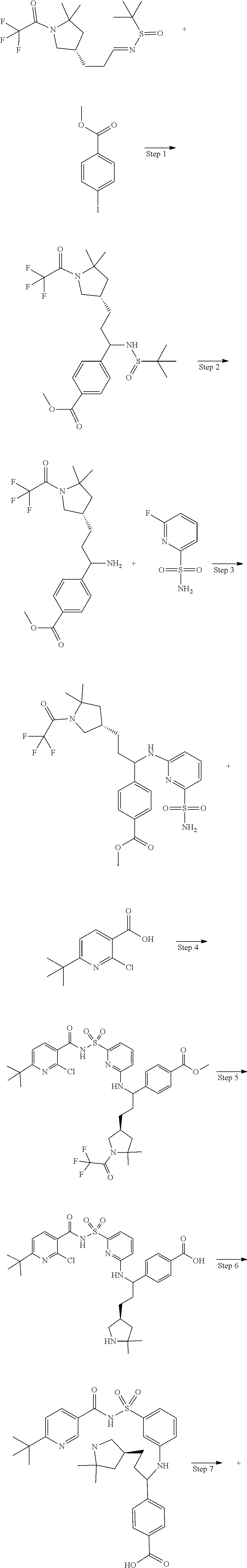

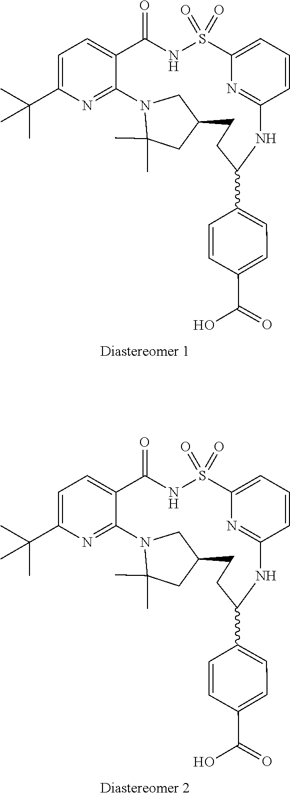 [SMILES: CC(C)(C)c1ccc2c(n1)N1C[C@@H](CCC(c3ccc(C(=O)O)cc3)Nc3cccc(n3)S(=O)(=O)NC2=O)CC1(C)C ; CC(C)(C)c1ccc2c(n1)N1C[C@@H](CCC(c3ccc(C(=O)O)cc3)Nc3cccc(n3)S(=O)(=O)NC2=O)CC1(C)C]

### Step 1: Methyl 4-[1-(tert-butylsulfinylamino)-3-[(3S)-5,5-dimethyl-1-(2,2,2-trifluoroacetyl)pyrrolidin-3-yl]propyl]benzoate

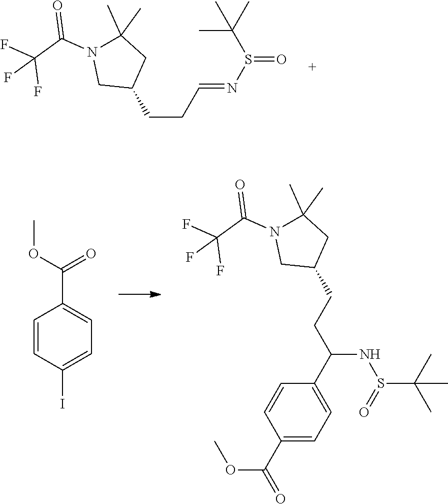 [SMILES: CC1(C)C[C@H](CC/C=N/S(=O)C(C)(C)C)CN1C(=O)C(F)(F)F ; COC(=O)c1ccc(I)cc1 ; COC(=O)c1ccc(C(CC[C@@H]2CN(C(=O)C(F)(F)F)C(C)(C)C2)NS(=O)C(C)(C)C)cc1]

Methyl 4-iodobenzoate (5.1 g, 19.073 mmol) was dissolved in THF (75 mL) and the clear solution was cooled in a dry ice/acetone bath (−23 to −19° C.) under nitrogen balloon. Isopropyl magnesium chloride (14.8 mL of 1.3 M in THF, 19.240 mmol) was then added in portions via syringe over 3 min. The resulting mixture (light brownish in color) was stirred within the same temperature range for 100 min. It was then cooled to −30° C. (NE)-N-[3-[(3S)-5,5-dimethyl-1-(2,2,2-trifluoroacetyl)pyrrolidin-3-yl]propylidene]-2-methyl-propane-2-sulfinamide (2.37 g, 6.3524 mmol) in solution in DCM (20 mL) was added slowly within 5 min. The mixture was further stirred at this temperature range for 2 h. Saturated aqueous NH₄Cl (30 mL) was added in one portion and the mixture was allowed to warm up to rt out of the cooling bath. Most volatiles were evaporated and the residue was partitioned between EtOAc and water (50 mL each). The layers were separated and the aqueous layer was extracted once with EtOAc (20 mL). The combined organics were dried over anhydrous MgSO₄, filtered and concentrated. The residue was purified by silica gel column, using 5-100% EtOAc in hexanes to afford methyl 4-[1-(tert-butylsulfinylamino)-3-[(3S)-5,5-dimethyl-1-(2,2,2-trifluoroacetyl)pyrrolidin-3-yl]propyl]benzoate (2.66 g, 81%). ESI-MS m/z calc. 490.2113, found 491.6 (M+1)⁺; Retention time: 3.32 minutes (LC method B).

### Step 2: Methyl 4-[1-amino-3-[(3S)-5,5-dimethyl-1-(2,2,2-trifluoroacetyl)pyrrolidin-3-yl]propyl]benzoate

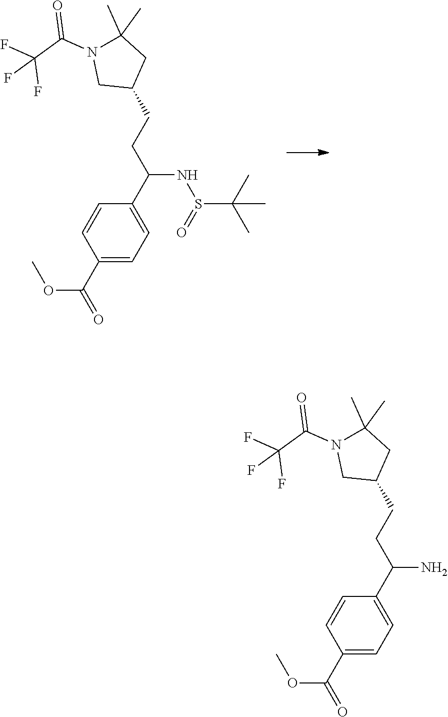 [SMILES: COC(=O)c1ccc(C(CC[C@@H]2CN(C(=O)C(F)(F)F)C(C)(C)C2)NS(=O)C(C)(C)C)cc1 ; COC(=O)c1ccc(C(N)CC[C@@H]2CN(C(=O)C(F)(F)F)C(C)(C)C2)cc1]

Methyl 4-[1-(tert-butyl sulfinyl amino)-3-[(3S)-5,5-dimethyl-1-(2,2,2-trifluoroacetyl)pyrrolidin-3-yl]propyl]benzoate (2.6 g, 5.0349 mmol) was dissolved in MeOH (30 mL) at rt. HCl (12 mL of 4 M, 48.000 mmol) in dioxane was added via pipette. The mixture was stirred at rt for 1 h. The mixture was concentrated in vacuo to remove most of the volatiles. The residue was partitioned between DCM and saturated aqueous sodium bicarbonate (40 mL each). The layers were separated and the aqueous layer was extracted once with DCM (15 mL). The combined organics were dried over anhydrous Na₂SO₄, filtered and concentrated to afford methyl 4-[1-amino-3-[(3S)-5,5-dimethyl-1-(2,2,2-trifluoroacetyl)pyrrolidin-3-yl]propyl]benzoate (1.9 g, 93%). ESI-MS m/z calc. 386.1817, found 387.5 (M+1)⁺; Retention time: 2.43 minutes (LC method B).

### Step 3: Methyl 4-[3-[(3S)-5,5-dimethyl-1-(2,2,2-trifluoroacetyl)pyrrolidin-3-yl]-1-[(6-sulfamoyl-2-pyridyl)amino]propyl]benzoate

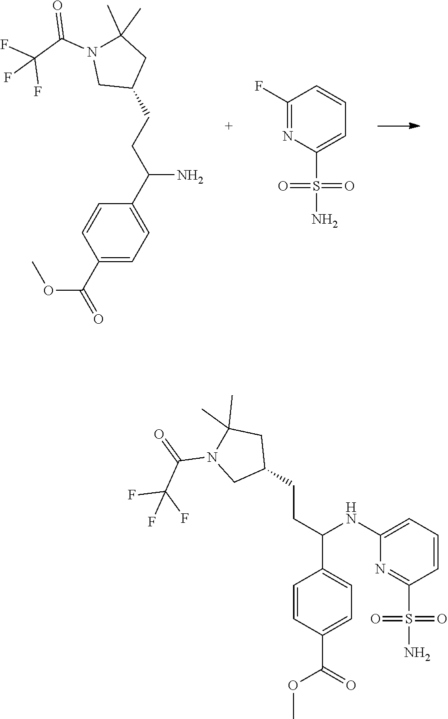 [SMILES: COC(=O)c1ccc(C(N)CC[C@@H]2CN(C(=O)C(F)(F)F)C(C)(C)C2)cc1 ; NS(=O)(=O)c1cccc(F)n1 ; COC(=O)c1ccc(C(CC[C@@H]2CN(C(=O)C(F)(F)F)C(C)(C)C2)Nc2cccc(S(N)(=O)=O)n2)cc1]

Methyl 4-[1-amino-3-[(3S)-5,5-dimethyl-1-(2,2,2-trifluoroacetyl)pyrrolidin-3-yl]propyl]benzoate (1.9 g, 4.671 mmol) was dissolved in DMSO (3 mL) at rt. 6-Fluoropyridine-2-sulfonamide (1.32 g, 7.493 mmol) was added, followed by Na₂CO₃ (1.5 g, 14.153 mmol). The mixture was heated in a 110° C. oil bath for 16 h. It was then cooled to rt, diluted with EtOAc (30 mL) and water (30 mL). The organic layer was further washed with water (20 mL×2), brine, dried over anhydrous MgSO₄, filtered and concentrated. The residue was purified by silica gel chromatography, using 20-80% EtOAc in hexanes to afford methyl 4-[3-[(3S)-5,5-dimethyl-1-(2,2,2-trifluoroacetyl)pyrrolidin-3-yl]-1-[(6-sulfamoyl-2-pyridyl)amino]propyl]benzoate (1.067 g, 40%). ¹H NMR (500 MHz, DMSO-d₆) δ 7.89 (d, J=8.2 Hz, 2H), 7.59 (d, J=8.3 Hz, 3H), 7.54-7.47 (m, 1H), 7.05 (s, 2H), 6.95 (d, J=7.2 Hz, 1H), 6.62 (d, J=8.4 Hz, 1H), 3.81 (m, 4H), 3.19-3.04 (m, 1H), 2.34-2.20 (m, 1H), 2.00-1.66 (m, 3H), 1.55-1.20 (m, 4H), 1.43 (s, 3H), 1.34-1.31 (s, 3H). ESI-MS m/z calc. 542.1811, found 543.4 (M+1)⁺; Retention time: 2.48 minutes (LC method H).

### Step 4: Methyl 4-[1-[[6-[(6-tert-butyl-2-chloro-pyridine-3-carbonyl)sulfamoyl]-2-pyridyl]amino]-3-[(3S)-5,5-dimethyl-1-(2,2,2-trifluoroacetyl)pyrrolidin-3-yl]propyl]benzoate

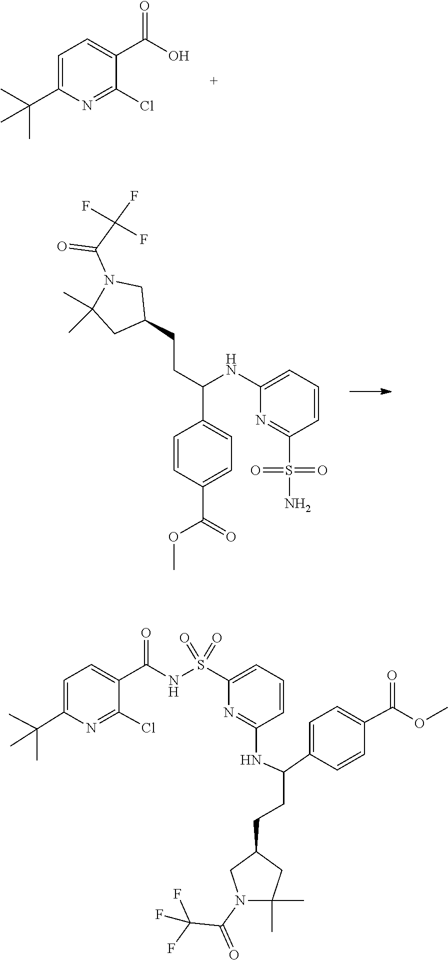 [SMILES: CC(C)(C)c1ccc(C(=O)O)c(Cl)n1 ; COC(=O)c1ccc(C(CC[C@@H]2CN(C(=O)C(F)(F)F)C(C)(C)C2)Nc2cccc(S(N)(=O)=O)n2)cc1 ; COC(=O)c1ccc(C(CC[C@@H]2CN(C(=O)C(F)(F)F)C(C)(C)C2)Nc2cccc(S(=O)(=O)NC(=O)c3ccc(C(C)(C)C)nc3Cl)n2)cc1]

To a solution of 6-tert-butyl-2-chloro-pyridine-3-carboxylic acid (125 mg, 0.585 mmol) in THF (853.8 μL) was added CDI (118.5 mg, 0.7308 mmol) (recrystallized from THF) and the mixture was stirred at rt for 1 h. Then methyl 4-[3-[(3S)-5,5-dimethyl-1-(2,2,2-trifluoroacetyl)pyrrolidin-3-yl]-1-[(6-sulfamoyl-2-pyridyl)amino]propyl]benzoate (332 mg, 0.612 mmol) was added as a solution in THF (312.5 μL) followed by DBU (279.7 mg, 1.837 mmol) and the resulting mixture was stirred for 3 h at room temperature. The reaction was diluted with water and EtOAc then aqueous HCl (654 μL of 6 M, 3.924 mmol) was added, the aqueous layer was then pH=1. The layers were separated and the organic layer was washed with water (1×) and brine (1×) then dried over sodium sulfate and concentrated to a white foam which was filtered and purified using a reverse phase HPLC-MS method using a Luna C₁₈ column (75×30 mm, 5 μm particle size) sold by Phenomenex (pn: 00C-4252-U0-AX), and a dual gradient run from 1-99% mobile phase B over 15.0 minutes (mobile phase A=H₂O (5 mM HCl), mobile phase B=acetonitrile, flow rate=50 mL/min, injection volume=950 μL and column temperature=25° C.) giving methyl 4-[1-[[6-[(6-tert-butyl-2-chloro-pyridine-3-carbonyl)sulfamoyl]-2-pyridyl]amino]-3-[(3S)-5,5-dimethyl-1-(2,2,2-trifluoroacetyl)pyrrolidin-3-yl]propyl]benzoate (282.7 mg, 65%). ESI-MS m/z calc. 737.2262, found 738.1 (M+1)⁺; Retention time: 0.79 minutes (LC method D).

### Step 5: 4-[1-[[6-[(6-tert-Butyl-2-chloro-pyridine-3-carbonyl)sulfamoyl]-2-pyridyl]amino]-3-[(3S)-5,5-dimethylpyrrolidin-3-yl]propyl]benzoic acid

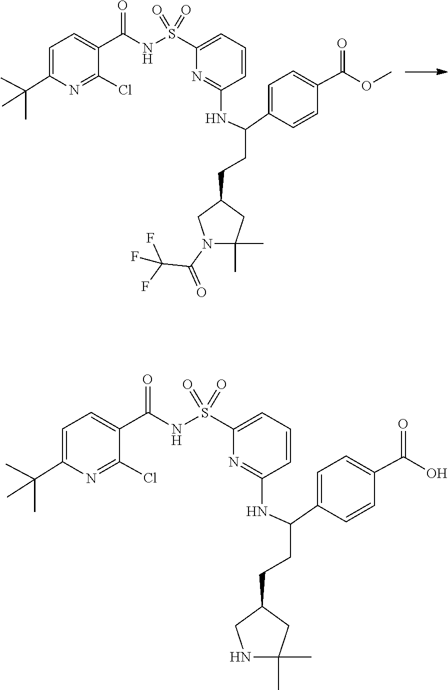

Methyl 4-[1-[[6-[(6-tert-butyl-2-chloro-pyridine-3-carbonyl)sulfamoyl]-2-pyridyl]amino]-3-[(3S)-5,5-dimethyl-1-(2,2,2-trifluoroacetyl)pyrrolidin-3-yl]propyl]benzoate (280 mg, 0.3793 mmol) and potassium carbonate (1.048 g, 7.583 mmol) were combined in a vial with isopropanol (2.8 mL), water (2.8 mL) and methanol (230.5 μL, 5.690 mmol). The resulting mixture was capped, heated to 100° C. and stirred overnight. It was cooled to rt, methanol (2 mL, 49.37 mmol) was added, then the mixture was capped, heated to 100° C. and stirred for 6 days. It was cooled to room temperature, diluted with water (50 mL) and 1N HCl (50 mL), then extracted with EtOAc (3 x, added a trace amount of MeOH to each extraction for solubility). The combined organic layers was dried over sodium sulfate, filtered and concentrated to a white solid, 4-[1-[[6-[(6-tert-butyl-2-chloro-pyridine-3-carbonyl)sulfamoyl]-2-pyridyl]amino]-3-[(3S)-5,5-dimethylpyrrolidin-3-yl]propyl]benzoic acid (238.3 mg, 100%). ESI-MS m/z calc. 627.2282, found 628.2 (M+1)⁺; Retention time: 0.52 minutes (LC method D).

### Step 6: 4-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]benzoic acid

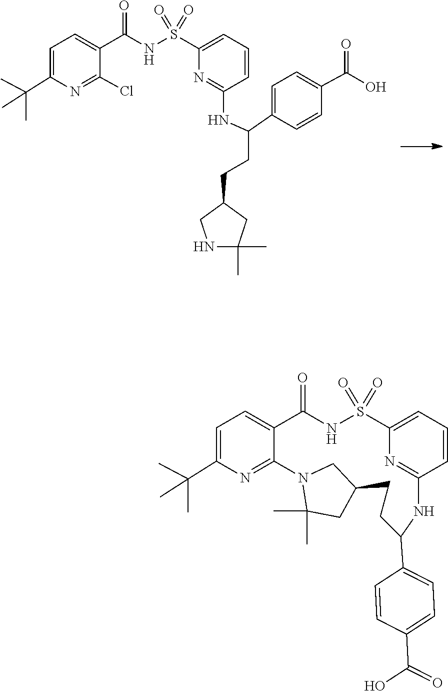 [SMILES: CC1(C)C[C@H](CCC(Nc2cccc(S(=O)(=O)NC(=O)c3ccc(C(C)(C)C)nc3Cl)n2)c2ccc(C(=O)O)cc2)CN1 ; CC(C)(C)c1ccc2c(n1)N1C[C@@H](CCC(c3ccc(C(=O)O)cc3)Nc3cccc(n3)S(=O)(=O)NC2=O)CC1(C)C]

To a solution of 4-[1-[[6-[(6-tert-butyl-2-chloro-pyridine-3-carbonyl)sulfamoyl]-2-pyridyl]amino]-3-[(3S)-5,5-dimethylpyrrolidin-3-yl]propyl]benzoic acid (238.3 mg, 0.3793 mmol) in NMP (14.3 mL) was added potassium carbonate (367.1 mg, 2.656 mmol). The mixture was purged with nitrogen for 5 min. The mixture was heated at 165° C. for 2 days. The mixture was cooled to room temperature, diluted with 1N HCl and extracted with EtOAc (2×). The organic phases were combined, dried (sodium sulfate), filtered and concentrated to a brown oil which was filtered and purified using a reverse phase HPLC-MS method using a Luna C₁₈ column (75×30 mm, 5 μm particle size) sold by Phenomenex (pn: 00C-4252-U0-AX), and a dual gradient run from 1-99% mobile phase B over 15.0 minutes (mobile phase A=H₂O (5 mM HCl), mobile phase B=acetonitrile, flow rate=50 mL/min, injection volume=9504, and column temperature=25° C.) giving 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]benzoic acid as a white solid (mixture of two diastereomeric products, 53.6 mg, 24%). ESI-MS m/z calc. 591.2515, found 592.2 (M+1)⁺; Retention time: 0.76 minutes (LC method D).

### Step 7: 4-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]benzoic acid (diastereomer 1), Compound 291, and 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]benzoic acid (diastereomer 2), Compound 290

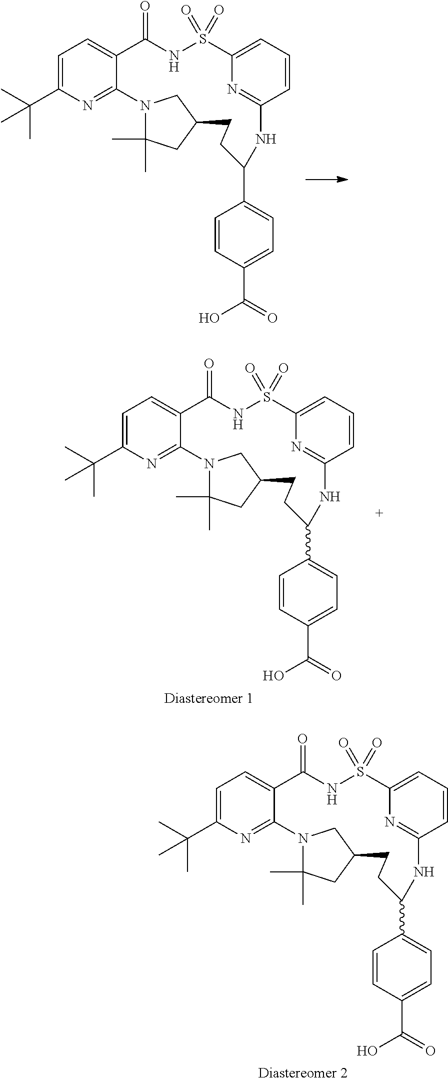

Subjected 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]benzoic acid (53.6 mg, 0.09058 mmol) to chiral separation by SFC chromatography using a Regis-[R,R]-Whelk-O (250×10 mm column, 5 μm particle size) column with 30% MeOH/70% CO₂ mobile phase at 10 mL/min over 10.0 minutes (injection volume=70 μL of 23 mg/mL solution in 90/10 MeOH/DMSO giving as the first diastereomer to elute 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]benzoic acid, diastereomer 1 (10.55 mg, 39%). ¹H NMR (500 MHz, DMSO-d₆) δ 12.85 (s, 1H), 12.53 (s, 1H), 7.88 (d, J=7.9 Hz, 2H), 7.74 (s, 1H), 7.65 (d, J=7.9 Hz, 2H), 7.54 (d, J=7.9 Hz, 2H), 7.10 (d, J=7.2 Hz, 1H), 6.84 (d, J=8.5 Hz, 1H), 6.66 (d, J=8.0 Hz, 1H), 5.26 (t, J=10.9 Hz, 1H), 3.25 (s, 1H), 2.79 (s, 1H), 2.30 (s, 1H), 1.92 (s, 1H), 1.84 (d, J=8.9 Hz, 1H), 1.75 (t, J=13.8 Hz, 2H), 1.66 (s, 3H), 1.58 (t, J=12.4 Hz, 1H), 1.52 (s, 3H), 1.47 (d, J=12.3 Hz, 1H), 1.29 (s, 9H). ESI-MS m/z calc. 591.2515, found 592.5 (M+1)⁺; Retention time: 2.05 minutes (LC method A); and as the second diastereomer to elute, 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]benzoic acid, diastereromer 2 (13.8 mg, 51%). ¹H NMR (500 MHz, DMSO-d₆) δ 12.85 (s, 1H), 12.35 (s, 1H), 7.89 (d, J=7.9 Hz, 2H), 7.74 (s, 1H), 7.62 (d, J=7.9 Hz, 3H), 7.50 (s, 1H), 7.21 (d, J=7.2 Hz, 1H), 6.78 (d, J=8.5 Hz, 1H), 6.64 (d, J=7.9 Hz, 1H), 4.89 (s, 1H), 3.17 (s, 1H), 3.04 (s, 1H), 2.55 (s, 1H), 2.31 (s, 1H), 1.85 (dd, J=11.7, 5.8 Hz, 2H), 1.73 (d, J=11.9 Hz, 1H), 1.60 (s, 3H), 1.54 (s, 3H), 1.50 (s, 1H), 1.28 (s, 9H), 1.15 (s, 1H). ESI-MS m/z calc. 591.2515, found 592.5 (M+1)⁺; Retention time: 2.0 minutes (LC method Q).

### Example 17: Preparation of (14S)-8-tert-Butyl-12,12-dimethyl-17-(2,2,6,6-tetramethyloxan-4-yl)-2λ⁶-thia-3,9,11,18,23-pentaazatetracyclo[17.3.1.111,14.05,10]tetracosa-1(23),5,7,9,19,21-hexaene-2,2,4-trione, Compound 42 (diastereomer 2) and Compound 43 (diastereomer 1)

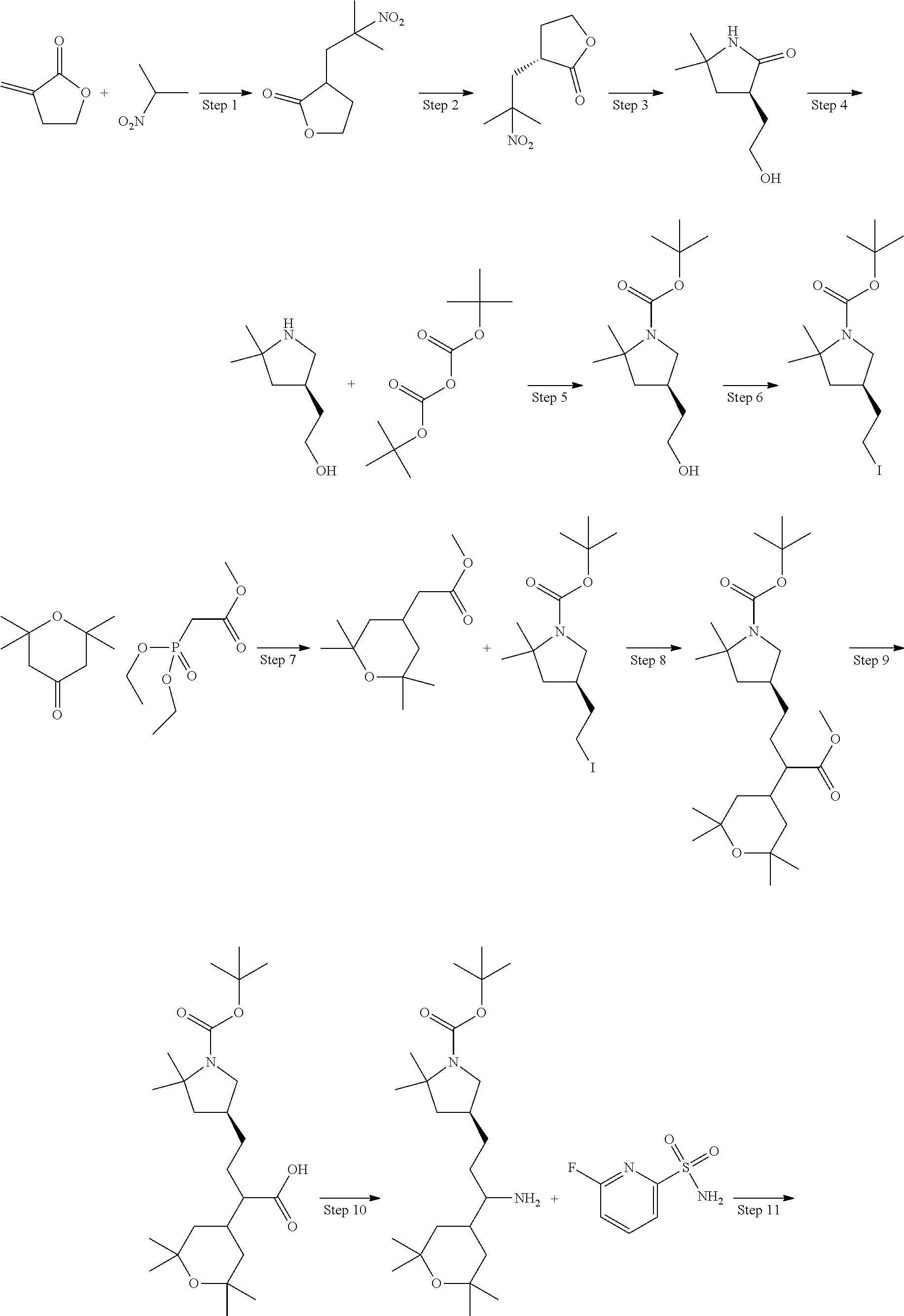

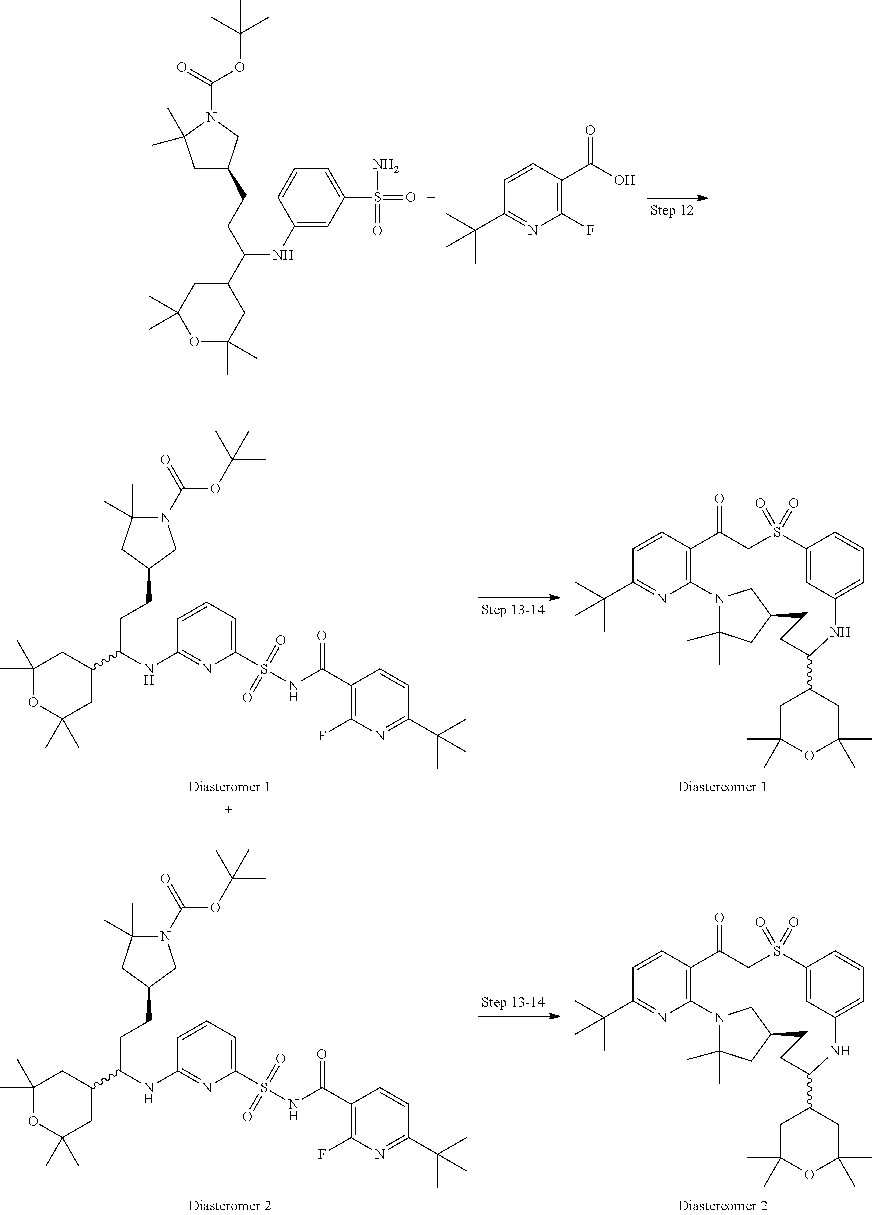

### Step 1: 3-(2-Methyl-2-nitro-propyl)tetrahydrofuran-2-one

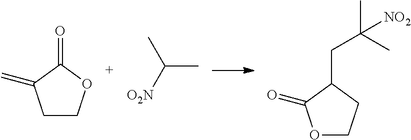

A solution of 3-methylenetetrahydrofuran-2-one (99.61 g, 974.78 mmol) dissolved in acetonitrile (1.1 L) was added in a slow stream to a mixture of DBU (22 mL, 147.11 mmol) and 2-nitropropane (105 mL, 1.169 mol). The reaction was exothermic: the addition rate was such that the internal temperature remained below 35° C. during the course of the addition. The resulting solution was stirred overnight at room temperature, then was concentrated under vacuum to obtain a light yellow solid. The crude product was stirred overnight in diethyl ether (500 mL), then filtered. The filtrate was discarded, and the resulting light yellow solid was dried under vacuum. LCMS detected some DBU residue. The crude was combined with for further purification. The solids were re-stirred in diethyl ether (500 mL), then filtered. The solids were dissolved in DCM (500 mL) and washed with 3.0 M aqueous HCl (2×500 mL), H₂O (500 mL), saturated aqueous sodium bicarbonate (500 mL), and saturated aqueous NaCl (500 mL). The DCM layer was dried over anhydrous magnesium sulfate, filtered and concentrated under vacuum to give 3-(2-methyl-2-nitro-propyl)tetrahydrofuran-2-one (144.69 g, 77%). ¹H NMR (500 MHz, DMSO-d₆) δ 4.28 (td, J=8.6, 1.5 Hz, 1H), 4.14-4.05 (m, 1H), 2.69 (dddd, J=11.5, 9.9, 8.6, 2.9 Hz, 1H), 2.44 (dd, J=14.8, 2.9 Hz, 1H), 2.34-2.25 (m, 1H), 2.11-2.00 (m, 1H), 1.86 (qd, J=11.7, 8.6 Hz, 1H), 1.60 (s, 3H), 1.58 (s, 3H). ESI-MS m/z calc. 187.0845, found 210.1 (M+Na)+; Retention time: 1.34 minutes (LC method B).

### Step 2: (3R)-3-(2-Methyl-2-nitro-propyl)tetrahydrofuran-2-one and (3S)-3-(2-methyl-2-nitro-propyl)tetrahydrofuran-2-one

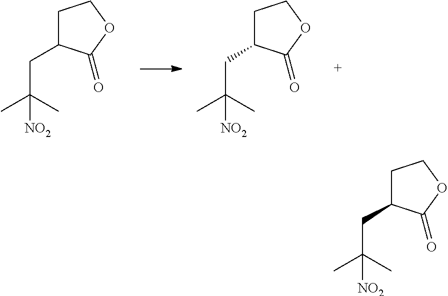

Racemic 3-(2-methyl-2-nitro-propyl)tetrahydrofuran-2-one (118.56 g, 633.4 mmol) was subjected to preparative chiral SFC using a ChiralPak IG (250×21.2 mm), 5 μm; Column; temperature: 40° C. at 32% MeOH (no modifier), 68% CO₂ with flow of 70 mL/min; concentrations: −32 mg/ml in MeOH (no modifier), Injection Volume 500 μL. Two isomers were isolated:

Peak 1: (3R)-3-(2-Methyl-2-nitro-propyl)tetrahydrofuran-2-one (55.8 g, 94%). ¹H NMR (400 MHz, DMSO-d₆) δ 4.27 (s, 1H), 4.09 (d, J=6.2 Hz, 1H), 2.69 (d, J=9.9 Hz, 1H), 2.43 (d, J=17.4 Hz, 1H), 2.29 (d, J=6.3 Hz, 1H), 2.06 (d, J=10.0 Hz, 1H), 1.85 (d, J=20.3 Hz, 1H), 1.59 (s, 3H), 1.58 (s, 3H).

Peak 2: (3S)-3-(2-Methyl-2-nitro-propyl)tetrahydrofuran-2-one (56.8 g, 96%). ¹H NMR (400 MHz, DMSO-d₆) δ 4.27 (s, 1H), 4.10 (d, J=16.5 Hz, 1H), 2.68 (d, J=10.0 Hz, 1H), 2.43 (d, J=14.8 Hz, 1H), 2.29 (s, 1H), 2.10-1.97 (m, 1H), 1.92-1.77 (m, 1H), 1.59 (s, 3H), 1.58 (s, 3H).

### Step 3: (3R)-3-(2-Hydroxyethyl)-5,5-dimethyl-pyrrolidin-2-one

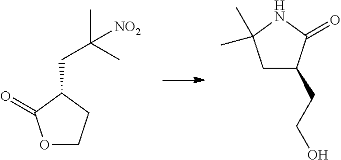

A mixture of (3R)-3-(2-methyl-2-nitro-propyl)tetrahydrofuran-2-one (35.7 g, 184.99 mmol), Raney Nickel (5.0 g, 57.019 mmol), and EtOH (350 mL) was stirred in a Parr Reactor at 80° C. under 3 bars of hydrogen for 24 h. Celite (10 g) was added and the resulting slurry was filtered. The solids were washed with ethanol (2×150 mL) and discarded. The combined filtrates were concentrated under vacuum to obtain a light brown solid. The resulting solid was dissolved in DCM (400 mL) and aqueous 1 M HCl (100 mL). The layers were separated. The aqueous layer was saturated and stirred with potassium chloride then was extracted with DCM (300 mL×3). The combined DCM layers were dried over sodium sulfate, filtered and concentrated under vacuum. The crude light brown solids (28.67 g) were triturated with diethyl ether (200 mL) overnight and filtered to afford white solids. (3R)-3-(2-hydroxyethyl)-5,5-dimethyl-pyrrolidin-2-one, (26.36 g, 86%). ESI-MS m/z calc. 157.1103, found 158.0 (M+1)⁺; Retention time: 1.48 minutes (LC method B).

### Step 4: 2-[(3R)-5,5-Dimethylpyrrolidin-3-yl]ethanol

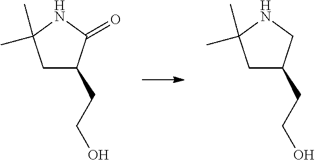

To a solution of (3R)-3-(2-hydroxyethyl)-5,5-dimethyl-pyrrolidin-2-one (26.36 g, 159.29 mmol) dissolved in anhydrous THF (280 mL) stirring at 0° C. was added portion-wise LAH (39.19 g, 42.737 mL, 980.93 mmol) to prevent excessive gas formation. The ice-bath was removed and the resulting gray slurry was stirred at 60° C. for 17 h. After allowing to cool to rt, the reaction was cooled to 0° C. H₂O (50 mL) was added slowly to prevent excessive gas formation and 15% NaOH in H₂O (50 mL) was added slowly. Another 120 mL of H₂O was added. The quenched mixture was diluted with diethyl ether (300 mL). The ice-bath was removed; the mixture was allowed to warm up to rt and stir for 30 min. The mixture was filtered through a small column of Celite. The solids were washed with diethyl ether (2×300 mL) and the combined filtrate was concentrated under vacuum to give 2-[(3R)-5,5-dimethylpyrrolidin-3-yl]ethanol (22.59 g, 94%). ¹H NMR (250 MHz, DMSO-d₆) δ 3.39-3.32 (m, 2H), 2.98-2.88 (m, 1H), 2.46-2.34 (m, 1H), 2.23-2.01 (m, 1H), 1.68 (dd, J=12.1, 8.2 Hz, 1H), 1.45 (qd, J=6.9, 2.0 Hz, 2H), 1.11-0.92 (m, 7H). ESI-MS m/z calc. 143.131, found 144.0 (M+1)⁺; Retention time: 1.15 minutes (LC method B).

### Step 5: tert-Butyl (4R)-4-(2-hydroxyethyl)-2,2-dimethyl-pyrrolidine-1-carboxylate

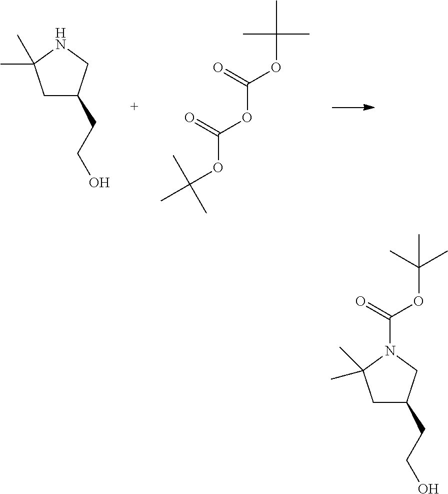

To a solution of 2-[(3R)-5,5-dimethylpyrrolidin-3-yl]ethanol (9.7462 g, 64.645 mmol) in Dioxane (100 mL) stirred at 0° C. was added aqueous NaOH (100 mL of 1 M, 100.00 mmol) and tert-butoxycarbonyl tert-butyl carbonate (17.55 g, 18.474 mL, 78.001 mmol) sequentially. The reaction was warmed to and stirred at rt for 2 h. The mixture was diluted with H₂O (200 mL) and extracted with EtOAc (3×250 mL). The organic layers were dried over anhydrous sodium sulfate, filtered and concentrated under vacuum. The crude oil was subjected to flash chromatography (loaded in DCM) (220 g SiO2, eluting 0 to 50% EtOAc/hexanes over 60 min.) to give tert-butyl (4R)-4-(2-hydroxyethyl)-2,2-dimethyl-pyrrolidine-1-carboxylate (11.781 g, 73%). ¹H NMR (250 MHz, DMSO-d₆) δ 4.48-4.39 (m, 1H), 3.65-3.44 (m, 1H), 2.86-2.67 (m, 1H), 2.14 (s, 1H), 1.94-1.79 (m, 1H), 1.53-1.10 (m, 20H). ESI-MS m/z calc. 243.1834, found 244.1 (M+1)⁺; Retention time: 2.48 minutes (LC method B).

### Step 6: tert-Butyl (4R)-4-(2-iodoethyl)-2,2-dimethyl-pyrrolidine-1-carboxylate

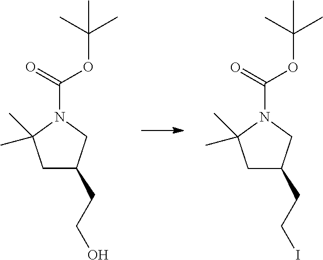 [SMILES: CC(C)(C)OC(=O)N1C[C@@H](CCO)CC1(C)C ; CC(C)(C)OC(=O)N1C[C@@H](CCI)CC1(C)C]

tert-Butyl (4R)-4-(2-hydroxyethyl)-2,2-dimethyl-pyrrolidine-1-carboxylate (2 g, 7.808 mmol) was dissolved in a solvent mixture of ether (15 mL) and acetonitrile (5 mL) and cooled in an ice water bath. Triphenylphosphine (6.15 g, 23.448 mmol) was added, followed by imidazole (1.6 g, 23.503 mmol) and molecular iodine (5.95 g, 23.443 mmol). The mixture was stirred at this an ice water bath for 1 h and slowly warmed up to rt and stirred for 4 h. It was then partitioned between ether and water. The organic layer was dried over anhydrous MgSO₄, filtered and concentrated to ¼ of its volume. Hexanes (40 mL) were added. The mixture was allowed to stand at rt for 15 h. It was then decanted. The residue was washed with a solvent mixture of ether/hexanes (1:3, v:v, total 20 mL) several times. The combined washings were concentrated. The residue was loaded on a 40 g column and purified using 0-30% EtOAc in hexanes gradient to afford tert-butyl (4R)-4-(2-iodoethyl)-2,2-dimethyl-pyrrolidine-1-carboxylate (2 g, 69%). ESI-MS m/z calc. 353.0852, found 354.5 (M+1)⁺; Retention time: 4.29 minutes as pale yellow oil (LC method B).

### Step 7: Methyl 2-(2,2,6,6-tetramethyltetrahydropyran-4-yl)acetate

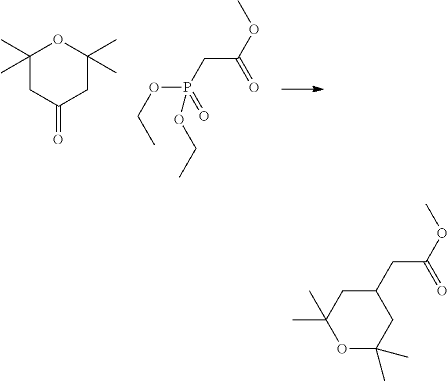

Methyl 2-diethoxyphosphorylacetate (5 g, 23.315 mmol) was dissolved in THF (80 mL) and cooled in an ice water bath under a nitrogen balloon. NaH (918 mg, 60% w/w, 22.952 mmol) was added in small portions. The mixture was stirred at this temperature for 30 min. 2,2,6,6-Tetramethyltetrahydropyran-4-one (2.8 g, 17.565 mmol) was added as a THF (5 mL) solution. The cooling bath was removed. The mixture was stirred at rt for 3 h before being placed in a 55° C. oil bath and stirred for 15 h. The mixture was cooled to rt. NH₄Cl (10 mL, saturated aqueous) was added. The mixture was extracted with EtOAc (80 mL) and water (100 mL). The organic layer was washed with brine, dried over anhydrous MgSO₄, filtered and concentrated. The residue was dissolved in MeOH (10 mL). Pd/C (10% w:w, 400 mg, 0.3759 mmol) was added. The mixture was purged with nitrogen and hydrogenated on a Parr shaker at 50 psi hydrogen pressure for 24 h. The catalyst was filtered off and the filtrate was concentrated and the residue was purified by silica gel chromatography (80 g column), using 0-40% EtOAc in hexanes to afford methyl 2-(2,2,6,6-tetramethyltetrahydropyran-4-yl)acetate (4 g, 96%). ¹H NMR (250 MHz, Chloroform-d) δ 3.69 (s, 3H), 2.21 (d, J=6.5 Hz, 2H), 1.66-1.52 (m, 2H), 1.27 (s, 6H), 1.19 (s, 6H), 1.15-0.85 (m, 3H).

### Step 8: tert-Butyl (4S)-4-[4-methoxy-4-oxo-3-(2,2,6,6-tetramethyltetrahydropyran-4-yl)butyl]-2,2-dimethyl-pyrrolidine-1-carboxylate

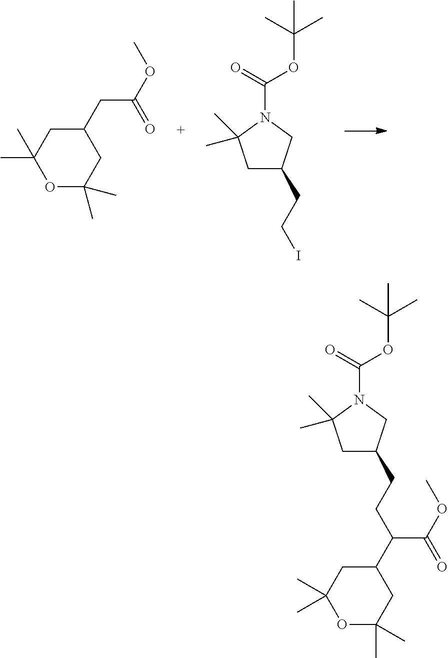 [SMILES: COC(=O)CC1CC(C)(C)OC(C)(C)C1 ; CC(C)(C)OC(=O)N1C[C@@H](CCI)CC1(C)C ; COC(=O)C(CC[C@@H]1CN(C(=O)OC(C)(C)C)C(C)(C)C1)C1CC(C)(C)OC(C)(C)C1]

Methyl 2-(2,2,6,6-tetramethyltetrahydropyran-4-yl)acetate (4 g, 16.799 mmol) was dissolved in THF (60 mL) and the mixture was cooled in a dry ice acetone bath under a nitrogen balloon and stirred for 15 min. LDA (9.5 mL of 2 M in THF/heptane/ethylbenzene, 19.00 mmol) was added. The mixture was stirred in the dry ice acetone bath for 1 h. tert-Butyl (4R)-4-(2-iodoethyl)-2,2-dimethyl-pyrrolidine-1-carboxylate (2 g, 5.379 mmol) in THF (5 mL) was then added. The mixture was stirred for 30 min and then allowed to warm up to 0° C. LC/MS showed little reaction. The mixture was cooled back in a dry ice acetone bath. LDA (0.8 mL of 2 M in THF/heptane/ethylbenzene, 1.41 mmol) was added. The mixture was allowed to warm up to rt under stirring for 15 h. NH₄Cl (10 mL, saturated aqueous) was added. The mixture was then partitioned between EtOAc (50 mL) and water (50 mL). The layers were separated and the aqueous layer was extracted with EtOAc (30 mL). The combined organics were dried over anhydrous MgSO₄, filtered and concentrated. The residue was purified by silica gel chromatography (40 g column), using 0-40% EtOAc in hexanes to afford tert-butyl (4S)-4-[4-methoxy-4-oxo-3-(2,2,6,6-tetramethyltetrahydropyran-4-yl)butyl]-2,2-dimethyl-pyrrolidine-1-carboxylate (2 g, 76%). ESI-MS m/z calc. 439.3298, found 440.6 (M+1)⁺; Retention time: 4.56 minutes as colorless oil (LC method B).

### Step 9: 4-[(3S)-1-tert-Butoxycarbonyl-5,5-dimethyl-pyrrolidin-3-yl]-2-(2,2,6,6-tetramethyltetrahydropyran-4-yl)butanoic acid

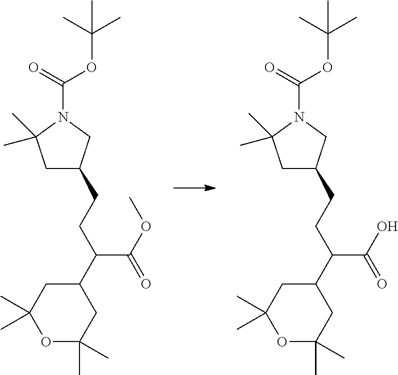 [SMILES: COC(=O)C(CC[C@@H]1CN(C(=O)OC(C)(C)C)C(C)(C)C1)C1CC(C)(C)OC(C)(C)C1 ; CC(C)(C)OC(=O)N1C[C@@H](CCC(C(=O)O)C2CC(C)(C)OC(C)(C)C2)CC1(C)C]

tert-Butyl (4S)-4-[4-methoxy-4-oxo-3-(2,2,6,6-tetramethyltetrahydropyran-4-yl)butyl]-2,2-dimethyl-pyrrolidine-1-carboxylate (2 g, 4.094 mmol) was dissolved in THF (15 mL). LiOH (1.72 g, 40.988 mmol) in water (5 mL) was added, followed by MeOH (5 mL). The mixture was heated in a 45° C. oil bath for 24 h. It was then cooled to rt, acidified with 1 N HCl (50 mL) and extracted with ether. The combined ether solution was washed with brine, filtered and concentrated to afford crude 4-[(3S)-1-tert-butoxycarbonyl-5,5-dimethyl-pyrrolidin-3-yl]-2-(2,2,6,6-tetramethyltetrahydropyran-4-yl)butanoic acid (1.5 g, 77%). ESI-MS m/z calc. 425.3141, found 426.6 (M+1)⁺; Retention time: 3.97 minutes (LC method B).

### Step 10: tert-Butyl (4S)-4-[3-amino-3-(2,2,6,6-tetramethyltetrahydropyran-4-yl)propyl]-2,2-dimethyl-pyrrolidine-1-carboxylate

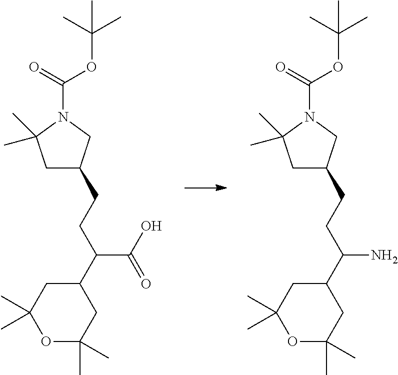 [SMILES: CC(C)(C)OC(=O)N1C[C@@H](CCC(C(=O)O)C2CC(C)(C)OC(C)(C)C2)CC1(C)C ; CC(C)(C)OC(=O)N1C[C@@H](CCC(N)C2CC(C)(C)OC(C)(C)C2)CC1(C)C]

4-[(3S)-1-tert-Butoxycarbonyl-5,5-dimethyl-pyrrolidin-3-yl]-2-(2,2,6,6-tetramethyltetrahydropyran-4-yl)butanoic acid (1.5 g, 3.1720 mmol) was dissolved in toluene (40 mL) at rt. TEA (544.50 mg, 0.75 mL, 5.3810 mmol) was added, followed by DPPA (0.70 mL, 3.093 mmol). The mixture was placed in a 90° C. oil bath and heated for 2 h. It was then cooled to rt and partitioned between EtOAc (40 mL) and water (40 mL). The organic layer was washed with water (30 mL) and brine. It was then concentrated. The residue was taken into THF (20 mL), then a solution of KOH (623 mg, 11.104 mmol) in water (10 mL) was added. The mixture was stirred at rt for 30 min. Most volatiles were removed under vacuum. The residue was taken into DCM (30 mL) and washed with water (20 mL×3). The DCM solution was then dried over anhydrous MgSO₄, filtered and concentrated. The crude material was used in the next step without further purification. tert-Butyl(4S)-4-[3-amino-3-(2,2,6,6-tetramethyltetrahydropyran-4-yl)propyl]-2,2-dimethyl-pyrrolidine-1-carboxylate (600 mg, 43%). ESI-MS m/z calc. 396.3352, found 397.6 (M+1)⁺; Retention time: 3.14 minutes (LC method B).

### Step 11: tert-Butyl (4S)-2,2-dimethyl-4-[3-[(6-sulfamoyl-2-pyridyl)amino]-3-(2,2,6,6-tetramethyltetrahydropyran-4-yl)propyl]pyrrolidine-1-carboxylate

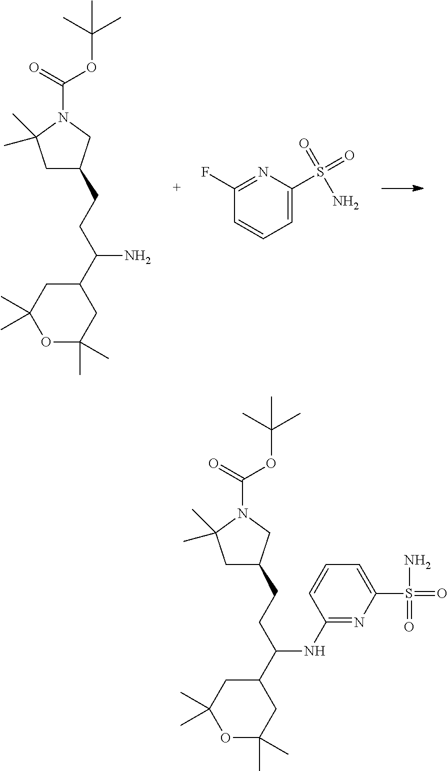 [SMILES: CC(C)(C)OC(=O)N1C[C@@H](CCC(N)C2CC(C)(C)OC(C)(C)C2)CC1(C)C ; NS(=O)(=O)c1cccc(F)n1 ; CC(C)(C)OC(=O)N1C[C@@H](CCC(Nc2cccc(S(N)(=O)=O)n2)C2CC(C)(C)OC(C)(C)C2)CC1(C)C]

tert-Butyl (4S)-4-[3-amino-3-(2,2,6,6-tetramethyltetrahydropyran-4-yl)propyl]-2,2-dimethyl-pyrrolidine-1-carboxylate (600 mg, 1.437 mmol) was dissolved in DMSO (2 mL). 6-Fluoropyridine-2-sulfonamide (355 mg, 2.015 mmol) was added, followed by Na₂CO₃ (503 mg, 4.746 mmol). The mixture was placed in a pre-heated 110° C. oil bath and stirred under a nitrogen balloon for 24 h. It was then cooled to rt and diluted with EtOAc/water (20 mL each). The layers were separated and the aqueous layer was extracted with EtOAc (20 mL). The combined organics was dried over anhydrous MgSO₄, filtered and concentrated. The residue was purified by silica gel chromatography (40 g column), using 0-50% EtOAc in hexanes to afford tert-butyl (4S)-2,2-dimethyl-4-[3-[(6-sulfamoyl-2-pyridyl)amino]-3-(2,2,6,6-tetramethyltetrahydropyran-4-yl)propyl]pyrrolidine-1-carboxylate (500 mg, 60%). ESI-MS m/z calc. 552.3345, found 553.5 (M+1)⁺; Retention time: 4.03 minutes (LC method B).

### Step 12: tert-Butyl (4S)-4-[3-[[6-[(6-tert-butyl-2-fluoro-pyridine-3-carbonyl)sulfamoyl]-2-pyridyl]amino]-3-(2,2,6,6-tetramethyltetrahydropyran-4-yl)propyl]-2,2-dimethyl-pyrrolidine-1-carboxylate, diastereomer 1 and tert-butyl (4S)-4-[3-[[6-[(6-tert-butyl-2-fluoro-pyridine-3-carbonyl)sulfamoyl]-2-pyridyl]amino]-3-(2,2,6,6-tetramethyltetrahydropyran-4-yl)propyl]-2,2-dimethyl-pyrrolidine-1-carboxylate, diastereomer 2

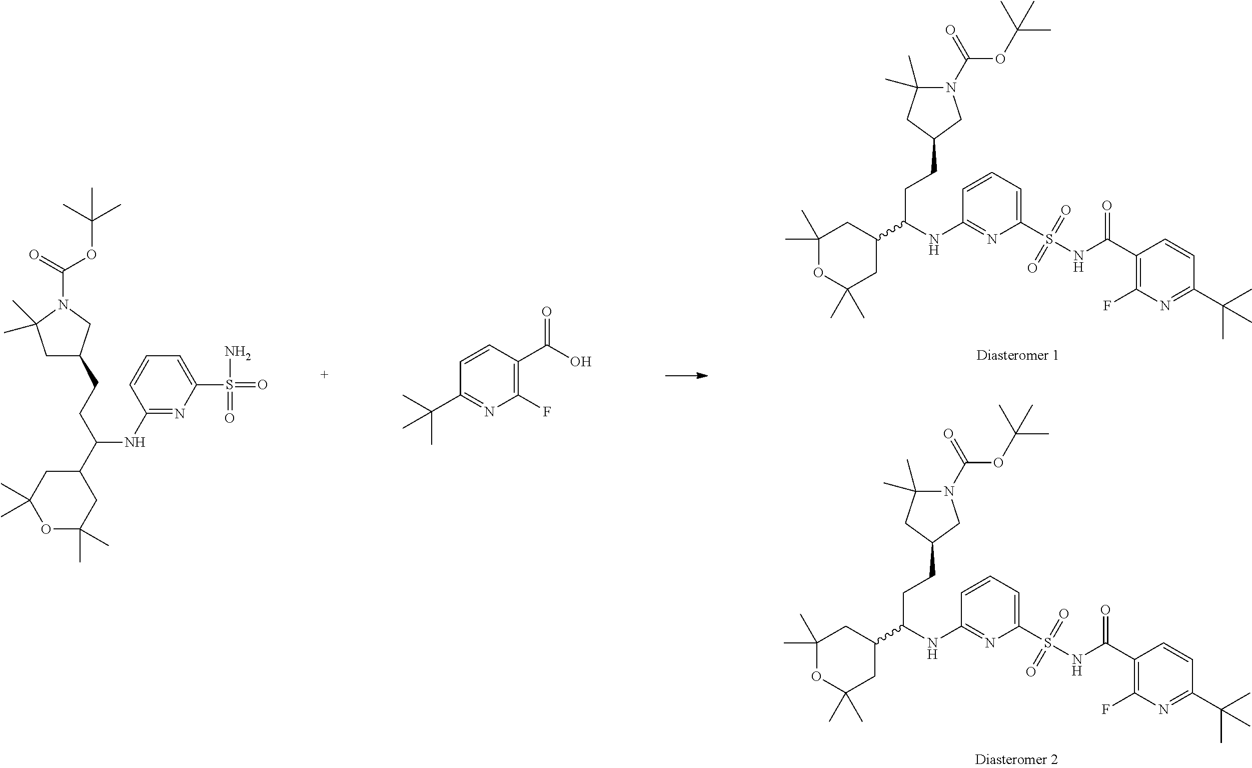 [SMILES: CC(C)(C)OC(=O)N1C[C@@H](CCC(Nc2cccc(S(N)(=O)=O)n2)C2CC(C)(C)OC(C)(C)C2)CC1(C)C ; CC(C)(C)c1ccc(C(=O)O)c(F)n1 ; CC(C)(C)OC(=O)N1C[C@@H](CCC(Nc2cccc(S(=O)(=O)NC(=O)c3ccc(C(C)(C)C)nc3F)n2)C2CC(C)(C)OC(C)(C)C2)CC1(C)C ; CC(C)(C)OC(=O)N1C[C@@H](CCC(Nc2cccc(S(=O)(=O)NC(=O)c3ccc(C(C)(C)C)nc3F)n2)C2CC(C)(C)OC(C)(C)C2)CC1(C)C]

6-tert-Butyl-2-fluoro-pyridine-3-carboxylic acid (688 mg, 3.489 mmol) was dissolved in THF (2 mL) at rt. CDI (564 mg, 3.478 mmol) was added in one portion. The mixture was stirred under a nitrogen balloon for 15 h. tert-Butyl (4S)-2,2-dimethyl-4-[3-[(6-sulfamoyl-2-pyridyl)amino]-3-(2,2,6,6-tetramethyltetrahydropyran-4-yl)propyl]pyrrolidine-1-carboxylate (500 mg, 0.859 mmol) was then added, immediately followed by DBU (0.52 mL, 3.4772 mmol).The reaction was quenched with a 1/1 mixture of saturated NH₄Cl and brine (5 mL each) and extracted with EtOAc. The combined organic layers were dried over anhydrous MgSO₄, filtered and concentrated. The residue was purified by reverse phase HPLC (Varian C₁₈ column 100 mm×30 mm, dual gradient run from 40-100% of mobile phase B, Mobile phase A=water (0.1% TFA), mobile phase B=Acetonitrile (0.1% TFA)) to afford two separated isomers:

Diastereomer 1: tert-Butyl (4S)-4-[3-[[6-[(6-tert-butyl-2-fluoro-pyridine-3-carbonyl)sulfamoyl]-2-pyridyl]amino]-3-(2,2,6,6-tetramethyltetrahydropyran-4-yl)propyl]-2,2-dimethyl-pyrrolidine-1-carboxylate (230 mg, 36%). ESI-MS m/z calc. 731.4092, found 732.9 (M+1)⁺; Retention time: 4.69 minutes (LC method B).

Diastereomer 2: tert-Butyl (4S)-4-[3-[[6-[(6-tert-butyl-2-fluoro-pyridine-3-carbonyl)sulfamoyl]-2-pyridyl]amino]-3-(2,2,6,6-tetramethyltetrahydropyran-4-yl)propyl]-2,2-dimethyl-pyrrolidine-1-carboxylate (290 mg, 44%). ESI-MS m/z calc. 731.4092, found 732.9 (M+1)⁺; Retention time: 4.83 minutes (LC method B).

### Step 13: 6-tert-Butyl-N-[[6-[[3-[(3S)-5,5-dimethylpyrrolidin-3-yl]-1-(2,2,6,6-tetramethyltetrahydropyran-4-yl)propyl]amino]-2-pyridyl]sulfonyl]-2-fluoro-pyridine-3-carboxamide, diastereomer 2

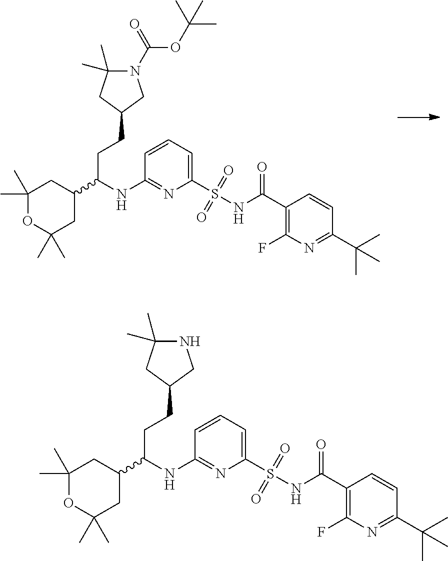

tert-Butyl (4S)-4-[3-[[6-[(6-tert-butyl-2-fluoro-pyridine-3-carbonyl)sulfamoyl]-2-pyridyl]amino]-3-(2,2,6,6-tetramethyltetrahydropyran-4-yl)propyl]-2,2-dimethyl-pyrrolidine-1-carboxylate, diastereomer 2 (290 mg, 0.3811 mmol) was dissolved in DCM (3 mL) at rt. TFA (1 mL, 12.980 mmol) was added in one portion. The mixture was stirred at rt for 30 min. It was then concentrated under vacuum to afford 6-tert-butyl-N-[[6-[[3-[(3S)-5,5-dimethylpyrrolidin-3-yl]-1-(2,2,6,6-tetramethyltetrahydropyran-4-yl)propyl]amino]-2-pyridyl]sulfonyl]-2-fluoro-pyridine-3-carboxamide (Trifluoroacetic Acid (1)) (380 mg, 127%) as a white foam. ESI-MS m/z calc. 631.3568, found 632.7 (M+1)⁺; Retention time: 3.45 minutes (LC method B).

### Step 14: (14S)-8-tert-Butyl-12,12-dimethyl-17-(2,2,6,6-tetramethyloxan-4-yl)-2λ⁶-thia-3,9,11,18,23-pentaazatetracyclo[17.3.1.111,14.05,10]tetracosa-1(23),5,7,9,19,21-hexaene-2,2,4-trione, Compound 42 (diastereomer 2)

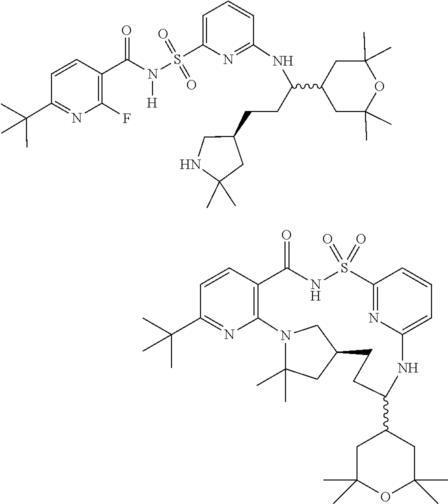

6-tert-Butyl-N-[[6-[[3-[(3S)-5,5-dimethylpyrrolidin-3-yl]-1-(2,2,6,6-tetramethyltetrahydropyran-4-yl)propyl]amino]-2-pyridyl]sulfonyl]-2-fluoro-pyridine-3-carboxamide (Trifluoroacetic Acid (1)) (380 mg, 0.4840 mmol) was dissolved in DMF (6 mL). K₂CO₃ (535 mg, 3.8710 mmol) was added. The mixture was heated in a 140° C. oil bath under a nitrogen balloon for 15 h. The mixture was cooled to rt, diluted with water (20 mL) and extracted with EtOAc (20 mL×2). The combined organics were concentrated and purified by silica gel chromatography (40 g column), using 0-40% EtOAc in hexanes to afford (14S)-8-tert-Butyl-12,12-dimethyl-17-(2,2,6,6-tetramethyloxan-4-yl)-2λ⁶-thia-3,9,11,18,23-pentaazatetracyclo[17.3.1.111,14.05,10]tetracosa-1(23),5,7,9,19,21-hexaene-2,2,4-trione, diastereomers 2, as a white solid (145 mg, 48%). ¹H NMR (500 MHz, DMSO-d₆) δ 12.46 (s, 1H), 7.62 (d, J=8.0 Hz, 1H), 7.59-7.53 (m, 1H), 7.03 (d, J=7.2 Hz, 1H), 6.88 (d, J=9.1 Hz, 1H), 6.73 (d, J=8.5 Hz, 1H), 6.64 (d, J=8.0 Hz, 1H), 3.90 (s, 1H), 3.08-2.94 (m, 1H), 2.61 (t, J=10.5, 10.5 Hz, 1H), 2.16-1.99 (m, 1H), 1.85-1.70 (m, 2H), 1.70-1.56 (m, 7H), 1.55-1.45 (m, 5H), 1.28 (s, 9H), 1.26-1.23 (m, 1H), 1.14 (d, J=7.9 Hz, 6H), 1.12-1.07 (m, 1H), 1.04 (d, J=8.7 Hz, 6H), 0.96 (t, J=12.7, 12.7 Hz, H). ESI-MS m/z calc. 611.3505, found 612.5 (M+1)⁺; Retention time: 3.41 minutes (LC method H).

The compound in the following table was prepared in a manner analogous to that described above, using tert-butyl (4S)-4-[3-[[6-[(6-tert-butyl-2-fluoro-pyridine-3-carbonyl)sulfamoyl]-2-pyridyl]amino]-3-(2,2,6,6-tetramethyltetrahydropyran-4-yl)propyl]-2,2-dimethyl-pyrrolidine-1-carboxylate, diastereomer 1, as a starting material:

| | | | LCMS | | | |
|---|---|---|---|---|---|---|
| | | | Retention | | | |
| Compound | | | Time | Exact | | LCMS |
| Number | Structure | NMR | (min) | Mass | M + 1 | Method |
| Compound 43 (Diastereomer 1) | 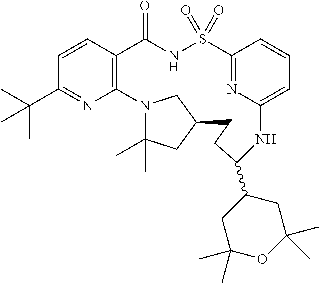 [SMILES: CC1(C)CC(C2CC[C@@H]3CN(c4nc(C(C)(C)C)ccc4C(=O)NS(=O)(=O)c4cccc(n4)N2)C(C)(C)C3)CC(C)(C)O1] | ¹H NMR (500 MHz, DMSO- d₆) δ 12.50 (s, 1H), 7.58 (t, J = 7.9, 7.9 Hz, 1H), 7.41 (d, J = 6.8 Hz, 1H), 7.17 (d, J = 7.2 Hz, 1H), 6.76 (d, J = 8.5 Hz, 1H), 6.61 (d, J = 7.9 Hz, 1H), 3.29-3.04 (m, 1H), 2.86-2.61 (m, 1H), 2.28- 2.18 (m, 1H), 1.87-1.76 (m, | 3.49 | 611.351 | 612.5 | LC method H |
| | | 1H), 1.74- | | | | |
| | | 1.40 (m, 12H), | | | | |
| | | 1.29-1.15 (m, | | | | |
| | | 15H), 1.09 (s, | | | | |
| | | 3H), 1.06 (s, | | | | |
| | | 6H), 0.98- | | | | |
| | | 0.89 (m, 2H). | | | | |

### Example 18: Preparation of (14S,17R)-17-(6-bromopyridin-2-yl)-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, Compound 215, and (14S,17S)-17-(6-bromopyridin-2-yl)-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, Compound 214

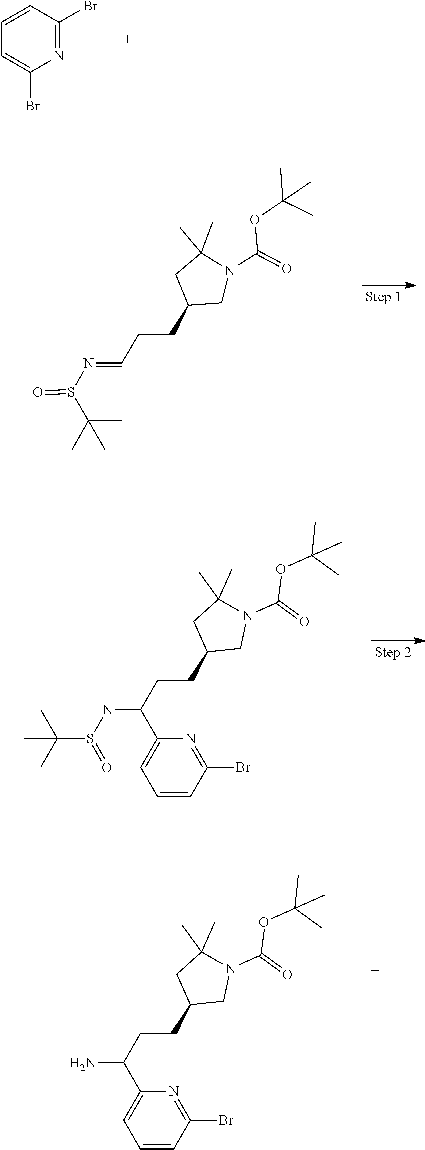

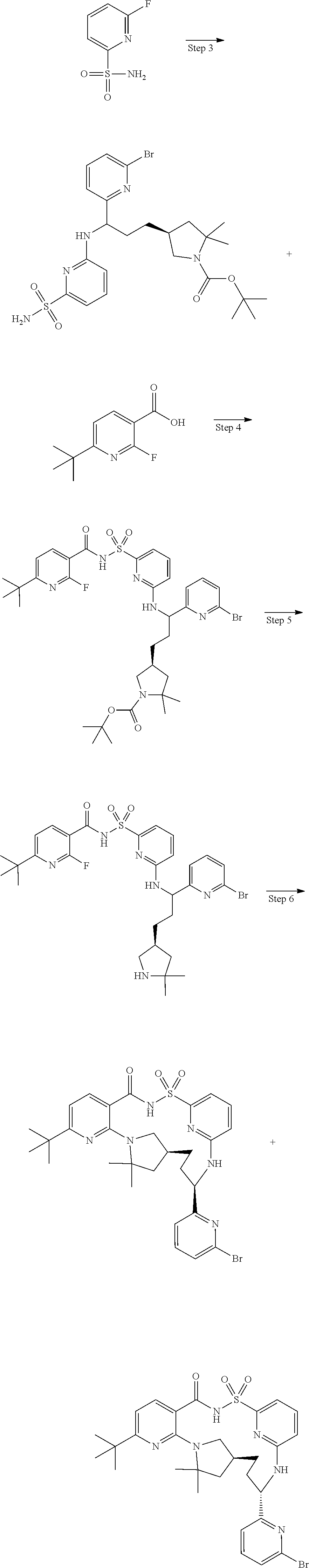

### Step 1: tert-Butyl (4S)-4-[3-(6-bromo-2-pyridyl)-3-(tert-butylsulfinylamino)propyl]-2,2-dimethyl-pyrrolidine-1-carboxylate

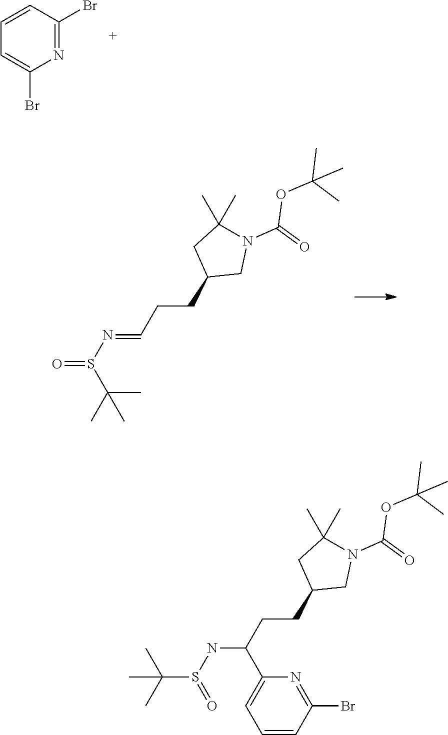

2,6-Dibromopyridine (12 g, 50.656 mmol) was dissolved in THF (100 mL). The solution was cooled in dry ice acetone bath and stirred under nitrogen balloon for 20 min. n-BuLi (20 mL of 2.5 M in hexanes, 50.00 mmol) was added quickly dropwise (exothermic reaction). The mixture was stirred in the dry ice acetone bath for 45 min. tert-Butyl (4S)-4-[(3E)-3-tert-butylsulfinyliminopropyl]-2,2-dimethyl-pyrrolidine-1-carboxylate (8 g, 22.313 mmol) in THF (20 mL plus 5 mL rinse) was added. After 15 min, NH₄Cl (30 mL, saturated aqueous) was added, followed by water (100 mL) and EtOAc (100 mL). The mixture was allowed to warm up to rt. The layers were separated and the organic layer was concentrated and the residue was purified by silica gel chromatography (120 g column), using 5-50% EtOAc in hexanes to afford tert-butyl (4S)-4-[3-(6-bromo-2-pyridyl)-3-(tert-butylsulfinylamino)propyl]-2,2-dimethyl-pyrrolidine-1-carboxylate (8 g, 62%). ESI-MS m/z calc. 515.1817, found 518.5 (M+1)⁺; Retention time: 3.86 minutes (LC method B).

### Step 2: tert-Butyl (4S)-4-[3-amino-3-(6-bromo-2-pyridyl)propyl]-2,2-dimethyl-pyrrolidine-1-carboxylate

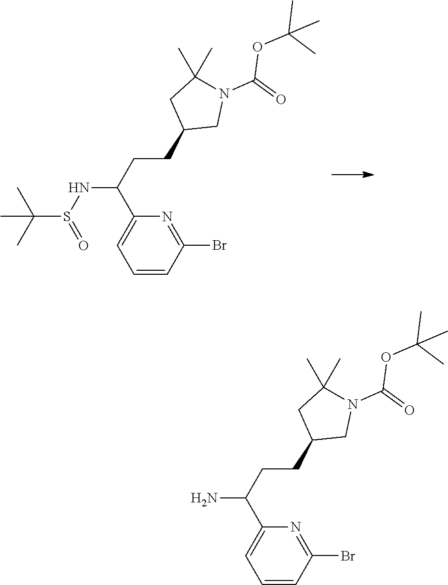 [SMILES: CC(C)(C)OC(=O)N1C[C@@H](CCC(NS(=O)C(C)(C)C)c2cccc(Br)n2)CC1(C)C ; CC(C)(C)OC(=O)N1C[C@@H](CCC(N)c2cccc(Br)n2)CC1(C)C]

tert-Butyl (4S)-4-[3-(6-bromo-2-pyridyl)-3-(tert-butylsulfinylamino)propyl]-2,2-dimethyl-pyrrolidine-1-carboxylate (8 g, 13.939 mmol) was dissolved in a solvent mixture of THF (100 mL) and water (20 mL). Molecular iodine (980 mg, 0.1988 mL, 3.8612 mmol) was added. The mixture was then heated in a 50° C. oil bath for 3 h. It was cooled to rt and diluted with EtOAc (100 mL) and NaS₂O₃ (10 g in 50 mL saturated aqueous sodium bicarbonate). The layers were separated. The organic layer was washed with brine (50 mL), dried over anhydrous Na₂SO₄, filtered and concentrated. The crude brown oil was used in the next step without further purification. tert-Butyl (4S)-4-[3-amino-3-(6-bromo-2-pyridyl)propyl]-2,2-dimethyl-pyrrolidine-1-carboxylate (7 g, 110%). ESI-MS m/z calc. 411.1521, found 414.5 (M+1)⁺; Retention time: 2.86 minutes (LC method B).

### Step 3: tert-Butyl (4S)-4-[3-(6-bromo-2-pyridyl)-3-[(6-sulfamoyl-2-pyridyl)amino]propyl]-2,2-dimethyl-pyrrolidine-1-carboxylate

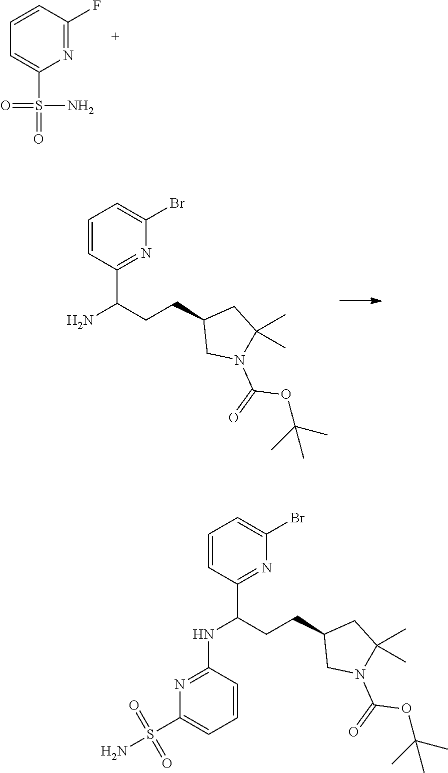

To a mixture of tert-butyl (4S)-4-[3-amino-3-(6-bromo-2-pyridyl)propyl]-2,2-dimethyl-pyrrolidine-1-carboxylate (6.28 g, 12.945 mmol) and 6-fluoropyridine-2-sulfonamide (4.5 g, 24.266 mmol) in DMSO (13 mL) was added DIEA (6.5 mL, 37.317 mmol). The mixture was stirred at 115° C. for 20 h. The reaction mixture was cooled to rt and then diluted with water (200 mL) and EtOAc (100 mL). The layers were separated and the organic layer was washed with brine (2×100 mL), dried over anhydrous Na₂SO₄, and concentrated. The residue was purified by silica gel chromatography (120 g column), using 0-80% EtOAc in hexanes to afford a pale color solid (7.8 g with purity about 60%) which was dissolved in DMSO (˜30 mL) for prep-HPLC (column: Varian C₁₈ 10 μm 5×30 cm; flow rate: 60 mL/min.; mobile phase A: water+0.1% TFA; mobile phase B: acetonitrile+0.1% TFA; method:0-45% B over 60 minutes). The pure fractions were combined and basified with saturated sodium bicarbonate and acetonitrile was removed. The product was extracted with DCM and the organic phase was washed with brine, dried over sodium sulfate and concentrated) to give tert-butyl (4S)-4-[3-(6-bromo-2-pyridyl)-3-[(6-sulfamoyl-2-pyridyl)amino]propyl]-2,2-dimethyl-pyrrolidine-1-carboxylate (4.15 g, 54%) as a white powder. ¹H NMR (500 MHz, DMSO-d₆) δ 7.67 (t, J=7.7, 7.7 Hz, 1H), 7.62-7.50 (m, 3H), 7.48 (d, J=7.7 Hz, 1H), 7.08 (s, 2H), 6.97 (d, J=7.2 Hz, 1H), 6.72 (d, J=10.1 Hz, 1H), 5.15 (bs, 1H), 3.59-3.47 (m, 1H), 2.84-2.68 (m, 1H), 2.15-2.02 (m, 1H), 1.96-1.77 (m, 3H), 1.46-1.31 (m, 15H), 1.23 (s, 3H). ESI-MS m/z calc. 567.1515, found 568.3 (M+1)⁺; Retention time: 2.72 minutes (LC method H).

### Step 4: tert-Butyl (4S)-4-[3-(6-bromo-2-pyridyl)-3-[[6-[(6-tert-butyl-2-fluoro-pyridine-3-carbonyl)sulfamoyl]-2-pyridyl]amino]propyl]-2,2-dimethyl-pyrrolidine-1-carboxylate

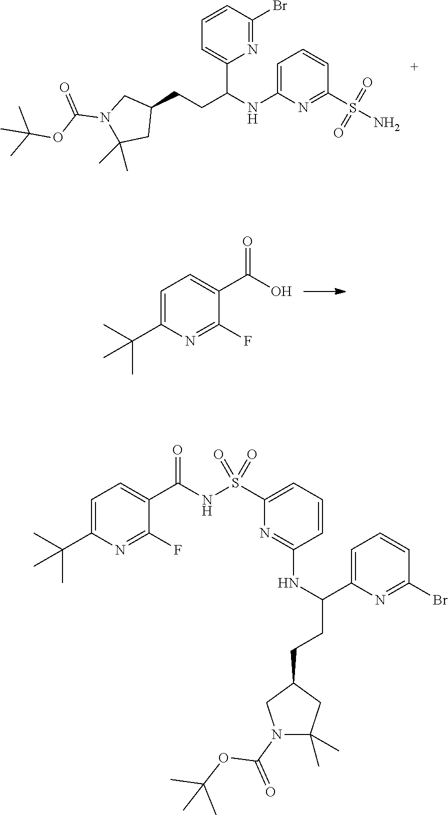 [SMILES: CC(C)(C)OC(=O)N1C[C@@H](CCC(Nc2cccc(S(N)(=O)=O)n2)c2cccc(Br)n2)CC1(C)C ; "+" ; CC(C)(C)c1ccc(C(=O)O)c(F)n1 ; CC(C)(C)OC(=O)N1C[C@@H](CCC(Nc2cccc(S(=O)(=O)NC(=O)c3ccc(C(C)(C)C)nc3F)n2)c2cccc(Br)n2)CC1(C)C]

To a solution of 6-tert-butyl-2-fluoro-pyridine-3-carboxylic acid (291.5 mg, 1.478 mmol) in THF (2.52 mL) was added CDI (245.7 mg, 1.515 mmol) (recrystallized from THF) and the mixture was stirred at rt for 5.5 h then tert-butyl (4S)-4-[3-(6-bromo-2-pyridyl)-3-[(6-sulfamoyl-2-pyridyl)amino]propyl]-2,2-dimethyl-pyrrolidine-1-carboxylate (420 mg, 0.7388 mmol) was added as a solution in THF (2.5 mL) followed by DBU (497.2 μL, 3.325 mmol) and the resulting mixture was stirred for 3 days at room temperature. The mixture was diluted with EtOAc and washed with 1N HCl (1×) followed by brine (1×), then dried (sodium sulfate), filtered and concentrated to an orange solid which was purified by silica gel chromatography using a shallow gradient from 100% hexanes to 100% EtOAc giving tert-butyl (4S)-4-[3-(6-bromo-2-pyridyl)-3-[[6-[(6-tert-butyl-2-fluoro-pyridine-3-carbonyl)sulfamoyl]-2-pyridyl]amino]propyl]-2,2-dimethyl-pyrrolidine-1-carboxylate (433 mg, 66%). ESI-MS m/z calc. 746.22614, found 749.7 (M+3)⁺; Retention time: 0.75 minutes (LC method I).

### Step 5: N-[[6-[[1-(6-Bromo-2-pyridyl)-3-[(3S)-5,5-dimethylpyrrolidin-3-yl]propyl]amino]-2-pyridyl]sulfonyl]-6-tert-butyl-2-fluoro-pyridine-3-carboxamide

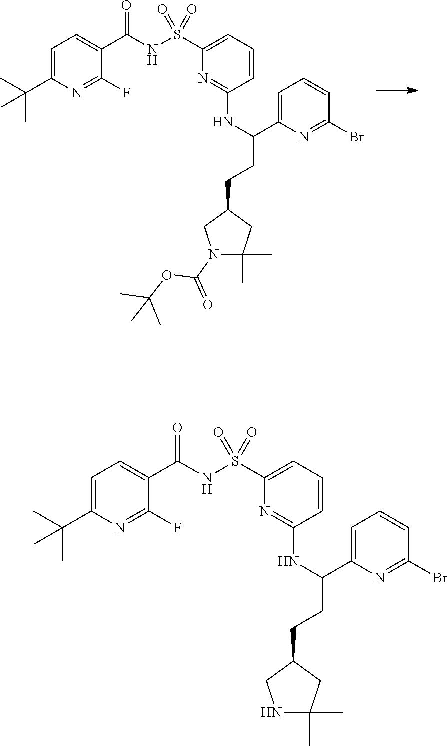 [SMILES: CC(C)(C)OC(=O)N1C[C@@H](CCC(Nc2cccc(S(=O)(=O)NC(=O)c3ccc(C(C)(C)C)nc3F)n2)c2cccc(Br)n2)CC1(C)C ; CC1(C)C[C@H](CCC(Nc2cccc(S(=O)(=O)NC(=O)c3ccc(C(C)(C)C)nc3F)n2)c2cccc(Br)n2)CN1]

tert-Butyl (4S)-4-[3-(6-bromo-2-pyridyl)-3-[[6-[(6-tert-butyl-2-fluoro-pyridine-3-carbonyl)sulfamoyl]-2-pyridyl]amino]propyl]-2,2-dimethyl-pyrrolidine-1-carboxylate (185 mg, 0.2474 mmol) was dissolved in DCM (807 μL) and to the mixture was added TFA (763 μL, 9.901 mmol) and the mixture was stirred at room temperature for 3 h. Concentrated mixture to dryness under reduced pressure, added 1 mL of toluene and removed by rotary evaporation (45° C. water bath). Again added 1 mL of toluene and removed by rotary evaporation (45° C. water bath) then dried on the high vacuum giving N-[[6-[[1-(6-bromo-2-pyridyl)-3-[(3S)-5,5-dimethylpyrrolidin-3-yl]propyl]amino]-2-pyridyl]sulfonyl]-6-tert-butyl-2-fluoro-pyridine-3-carboxamide (Trifluoroacetate salt) (188.4 mg, 100%).

### Step 6: (14S,17R)-17-(6-Bromopyridin-2-yl)-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, Compound 215. and (14S,17S)-17-(6-bromopyridin-2-yl)-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, Compound 214

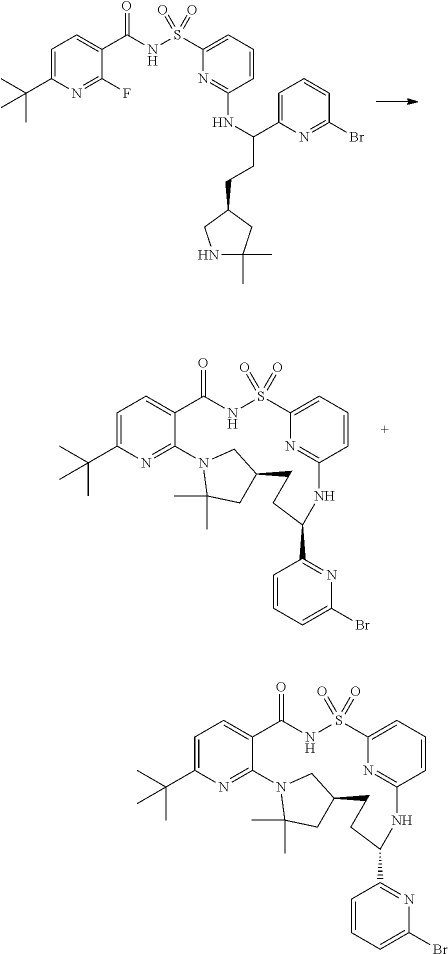

To a solution of N-[[6-[[1-(6-bromo-2-pyridyl)-3-[(3S)-5,5-dimethylpyrrolidin-3-yl]propyl]amino]-2-pyridyl]sulfonyl]-6-tert-butyl-2-fluoro-pyridine-3-carboxamide (Trifluoroacetate salt) (188.4 mg, 0.2474 mmol) in NMP (11.31 mL) was added potassium carbonate (239.5 mg, 1.733 mmol). The mixture was purged with nitrogen for 5 min. The mixture was heated at 165° C. for 16 h. The mixture was cooled to room temperature, diluted with 1N HCl and extracted with EtOAc (1×). The organic phase was washed with brine (1×), dried (sodium sulfate), filtered and concentrated to a brown oil which was filtered and purified using a reverse phase HPLC-MS method using a Luna C₁₈ column (75×30 mm, 5 μm particle size) sold by Phenomenex (pn: 00C-4252-U0-AX), and a dual gradient run from 50-99% mobile phase B over 15.0 minutes (mobile phase A=H₂O (5 mM HCl), mobile phase B=acetonitrile, flow rate=50 mL/min, injection volume=950 μL and column temperature=25° C.) giving as the first diastereomer to elute, (14S,17R)-17-(6-bromopyridin-2-yl)-8-tert-butyl-12,12-dimethyl-2λ⁶-thia-3,9,11,18,23-pentaazatetracyclo[17.3.1.111,14.05,10]tetracosa-1 (22),5,7,9,19(23),20-hexaene-2,2,4-trione as a white solid (37.3 mg, 46%). ESI-MS m/z calc. 626.1675, found 629.5 (M+1)⁺; Retention time: 2.24 minutes and as the second diastereomer to elute, (14S,17S)-17-(6-bromopyridin-2-yl)-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione as a white solid (35 mg, 45%). ESI-MS m/z calc. 626.1675, found 629.5 (M+1)⁺; Retention time: 2.27 minutes.

Diastereomer 1: (14S,17R)-17-(6-Bromopyridin-2-yl)-8-tert-butyl-12,12-dimethyl-2λ⁶-thia-3,9,11,18,23-pentanzatetracyclo[17.3.1.111,14.05,10]tetracosa-1(22),5,7,9,19(23),20-hexaene-2,2,4-trione, ¹H NMR (500 MHz, DMSO-d₆) δ 12.47 (s, 1H), 7.77-7.61 (m, 4H), 7.52-7.47 (m, 2H), 7.13 (dd, J=7.2, 0.7 Hz, 1H), 6.90 (dd, J 8.5, 0.8 Hz, 1H), 6.66 (d, J=8.0 Hz, 1H), 5.29-5.16 (m, 1H), 3.17 (dd, J=10.8, 6.2 Hz, 1H), 2.79-2.68 (m, 1H), 2.22 (s, 1H), 1.92 (d, J=6.0 Hz, 2H), 1.85 (dd, J=11.6, 5.2 Hz, 1H), 1.75 (d, J=14.3 Hz, 1H), 1.66 (s, 3H), 1.58 (t, J=12.4 Hz, 1H), 1.51 (s, 3H), 1.49-1.40 (m, 1H), 1.29 (s, 9H), ESI-MS m/z calc. 626.1675, found 629.5 (M+1)⁺; Retention time: 2.24 minutes (LC method A).

Diastereomer 2: (14S,17S)-17-(6-Bromopyridin-2-yl)-8-tert-butyl-12,12-dimethyl-2λ⁶-thia-3,9,11,18,23-pentanzatetracyclo[17.3.1.111,14.05,10]tetracosa-1(22),5,7,9,19(23),20-hexaene-2,2,4-trione, ¹H NMR (500 MHz, DMSO-d₆) δ 12.30 (s, 1H), 7.79 (s, 1H), 7.72 (t, J=7.8 Hz, 1H), 7.63 (dt, J=16.0, 7.8 Hz, 2H), 7.52 (d, J=7.9 Hz, 2H), 7.23 (d, J=7.2 Hz, 1H), 6.86 (d, J=8.5 Hz, 1H), 6.64 (d, J=7.8 Hz, 1H), 4.93 (s, 1H), 3.12 (s, 1H), 2.98 (s, 1H), 2.33 (s, 1H), 2.20-2.08 (m, 1H), 1.87 (dd, J=12.0, 6.0 Hz, 1H), 1.75 (d, J=12.6 Hz, 1H), 1.60 (s, 3H), 1.53 (s, 3H), 1.51 (s, 1H), 1.37-1.30 (m, 1H), 1.28 (s, 9H), 1.16 (d, J=11.9 Hz, 1H). ESI-MS m/z calc. 626.1675, found 629.5 (M+1)⁺; Retention time: 2.27 minutes (LC method A).

### Example 19: Preparation of (14S,17R)-8-tert-Butyl-12,12-dimethyl-17-{1′,2′,3′,6′-tetrahydro-[2,4′-bipyridine]-6-yl}-2λ⁶-thia-3,9,11,18,23-pentaazatetracyclo [17.3.1.111,14.05,10]tetracosa-1(23),5(10),6,8,19,21-hexaene-2,2,4-trione, Compound 142

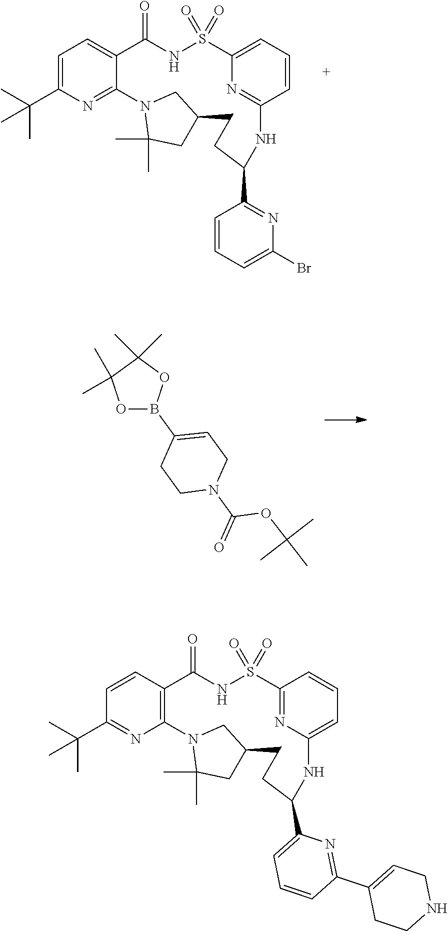

Combined (14S,17R)-17-(6-bromopyridin-2-yl)-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (30.2 mg, 0.0456 mmol), tert-butyl 4-(4,4,5,5-tetramethyl-1,3,2-dioxaborolan-2-yl)-3,6-dihydro-2H-pyridine-1-carboxylate (21.16 mg, 0.0684 mmol), potassium carbonate (31.97 mg, 0.231 mmol), Pd(dppf)Cl₂ (3.7 mg, 0.0045 mmol), DMSO (867 μL) and water (114 μL) in a vial and bubbled nitrogen through the mixture for 2 minutes. The reaction was capped and heated to 120° C. for 2 h. Cooled to room temperature, diluted with 1N HCl and extracted with EtOAc (3×). Combined the organic fractions, dried (sodium sulfate), filtered and concentrated to a residue which was filtered and purified using a reverse phase HPLC-MS method using a Luna C₁₈ column (75×30 mm, 5 μm particle size) sold by Phenomenex (pn: 00C-4252-U0-AX), and a dual gradient run from 30-99% mobile phase B over 15.0 minutes (mobile phase A=H₂O (5 mM HCl), mobile phase B=acetonitrile, flow rate=50 mL/min, injection volume=950 μL and column temperature=25° C.) giving a white solid which was dissolved in dichloromethane (573 μL) followed by addition of TFA (208 mg, 1.825 mmol). The resulting mixture was stirred for 1 h then volatiles were removed by rotary evaporation. The resulting residue was filtered and purified using a reverse phase HPLC-MS method using a Luna C₁₈ column (75×30 mm, 5 μm particle size) sold by Phenomenex (pn: 00C-4252-U0-AX), and a dual gradient run from 30-99% mobile phase B over 15.0 minutes (mobile phase A=H₂O (5 mM HCl), mobile phase B=acetonitrile, flow rate=50 mL/min, injection volume=950 μL and column temperature=25° C.) giving as a white solid, (14S,17R)-8-tert-butyl-12,12-dimethyl-17-11′,2′,3′,6′-tetrahydro-[2,4′-bipyridine]-6-yl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (9.9 mg, 33%). ESI-MS m/z calc. 629.3148, found 630.7 (M+1)⁺; Retention time: 1.73 minutes (LC method A).

### Example 20: Preparation of (14S,17R)-8-tert-butyl-12,12-dimethyl-17-[6-(piperidin-4-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione, Compound 132

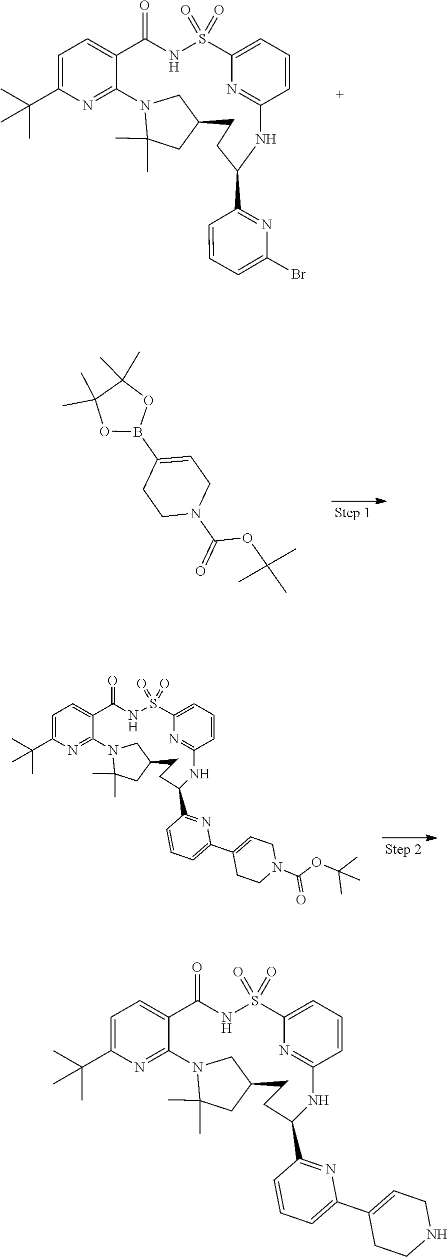

### Step 1: tert-Butyl 6-[(14S,17R)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-1′,2′,3′,6′-tetrahydro-[2,4′-bipyridine]-1′-carboxylate

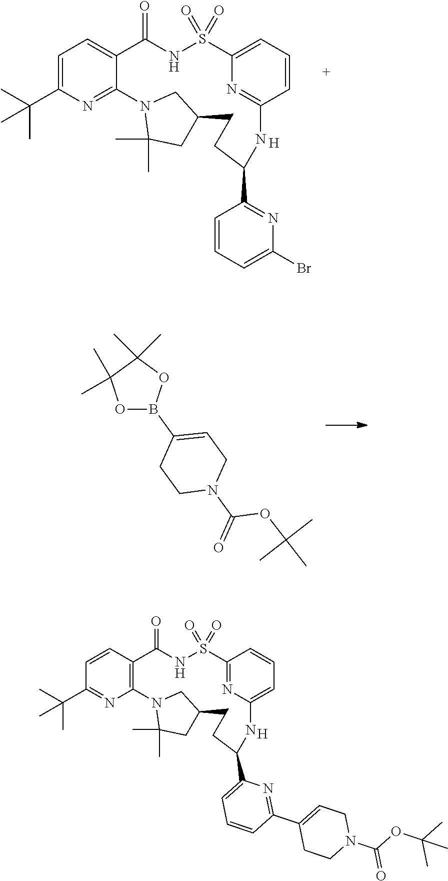

Combined (14S,17R)-17-(6-bromopyridin-2-yl)-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (20.5 mg, 0.0310 mmol), tert-butyl 4-(4,4,5,5-tetramethyl-1,3,2-dioxaborolan-2-yl)-3,6-dihydro-2H-pyridine-1-carboxylate (14.37 mg, 0.0465 mmol), potassium carbonate (21.7 mg, 0.157 mmol), Pd(dppf)Cl₂ (2.5 mg, 0.0031 mmol), DMSO (589 μL) and water (78 μL) in a vial and bubbled nitrogen through the mixture for 2 minutes. The reaction was capped and heated to 120° C. for 2 h. Cooled to room temperature, diluted with 1N HCl and extracted with EtOAc (3×). Combined the organic fractions, dried (sodium sulfate), filtered and concentrated to a residue which was filtered and purified using a reverse phase HPLC-MS method using a Luna C₁₈ column (75×30 mm, 5 μm particle size) sold by Phenomenex (pn: 00C-4252-U0-AX), and a dual gradient run from 30-99% mobile phase B over 15.0 minutes (mobile phase A=H₂O (5 mM HCl), mobile phase B=acetonitrile, flow rate=50 mL/min, injection volume=950 μL and column temperature=25° C.) giving as a white solid, tert-butyl 6-[(14S,17R)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-1′,2′,3′,6′-tetrahydro-[2,4′-bipyridine]-1′-carboxylate (hydrochloride salt) (22.1 mg, 93%). ESI-MS m/z calc. 729.36725, found 730.9 (M+1)⁺; Retention time: 0.86 minutes (LC method D).

### Step 2: (14S,17R)-8-tert-Butyl-12,12-dimethyl-17-[6-(piperidin-4-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione, Compound 132

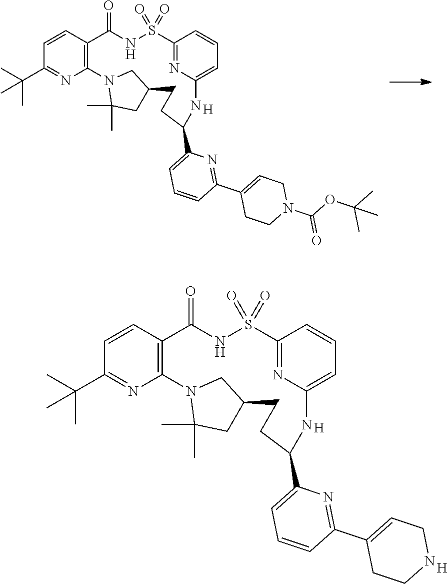

Dissolved a mixture of tert-butyl 6-[(14S,17R)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-1′,2′,3′,6′-tetrahydro-[2,4′-bipyridine]-r-carboxylate (hydrochloride salt) (22.1 mg, 0.0288 mmol) and 10% palladium on carbon (15.35 mg, 0.01442 mmol) in ethanol (1 mL) and bubbled nitrogen through the mixture for 2 min. Bubbled hydrogen through the mixture for 2 min then capped with an hydrogen balloon and stirred for 3 h. Purged the reaction vessel with nitrogen gas then filtered over a pad of celite followed by micro-filtration through a 0.2 μM frit. Concentrated the filtrate to give a residue which was then dissolved in DCM (500 μL) and TFA (89 μL, 1.154 mmol) was added. The resulting mixture was stirred for 1 h then volatiles were removed by rotarty evaporation. The residue was dissolved in DMSO, filtered and purified using a reverse phase HPLC-MS method using a Luna C₁₈ column (75×30 mm, 5 μm particle size) sold by Phenomenex (pn: 00C-4252-U0-AX), and a dual gradient run from 30-99% mobile phase B over 15.0 minutes (mobile phase A=H₂O (5 mM HCl), mobile phase B=acetonitrile, flow rate=50 mL/min, injection volume=950 μL and column temperature=25° C.) giving as a white solid, (14S,17R)-8-tert-butyl-12,12-dimethyl-17-[6-(piperidin-4-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (6 mg, 31%). ¹H NMR (400 MHz, DMSO-d₆) δ 12.49 (s, 1H), 8.84 (s, 1H), 8.49 (s, 1H), 7.73 (d, J=10.0 Hz, 1H), 7.69-7.59 (m, 3H), 7.31 (s, 1H), 7.14 (t, J=7.1 Hz, 2H), 6.92 (d, J=8.5 Hz, 1H), 6.65 (d, J=8.0 Hz, 1H), 5.20 (s, 1H), 3.36 (d, J=12.6 Hz, 2H), 3.15 (s, 1H), 3.06-2.92 (m, 3H), 2.75 (t, J=10.5 Hz, 1H), 2.28-2.08 (m, 2H), 2.02 (dd, J=20.1, 10.5 Hz, 2H), 1.89 (dtd, J=25.2, 13.1, 5.1 Hz, 4H), 1.74 (d, J=14.5 Hz, 1H), 1.66 (s, 3H), 1.59 (t, J=12.3 Hz, 1H), 1.51 (s, 3H), 1.49-1.39 (m, 1H), 1.28 (s, 9H). ESI-MS m/z calc. 631.33044, found 632.7 (M+1)⁺; Retention time: 1.5 minutes (LC method A).

The following is a list of boron reagents that are commercially available:
(Dimethylamino)methyl-trifluoro-boranuide, potassium salt
1H-Pyrazol-3-ylboronic acid
2-(3,6-Dihydro-2H-pyran-4-yl)-4,4,5,5-tetramethyl-1,3,2-dioxaborolane
4-(4,4,5,5-Tetramethyl-1,3,2-dioxaborolan-2-yl)-1H-pyrazole
3-(4,4,5,5-Tetramethyl-1,3,2-dioxaborolan-2-yl)propanenitrile
tert-Butyl 3-(4,4,5,5-tetramethyl-1,3,2-dioxaborolan-2-yl)azetidine-1-carboxylate
tert-Butyl N-[2-(4,4,5,5-tetramethyl-1,3,2-dioxaborolan-2-yl)ethyl]carbamate

The compounds in the following tables were prepared in a manner analogous to that described above using commercially available boron reagents given in the table above.

| | | LCMS | | | |
|---|---|---|---|---|---|
| | | Retention | | | |
| Compound | | Time | Exact | | LCMS |
| Number | Molecule | (min) | Mass | M + 1 | Method |
| Compound 99, hydrochloride salt | 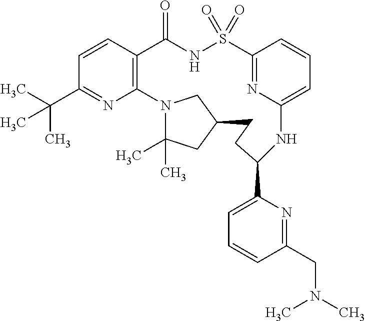 [SMILES: CN(C)Cc1cccc([C@H]2CC[C@@H]3CN(c4nc(C(C)(C)C)ccc4C(=O)NS(=O)(=O)c4cccc(n4)N2)C(C)(C)C3)n1] | 1.59 | 605.315 | 606.7 | LC method A |
| Compound 77, hydrochloride salt | 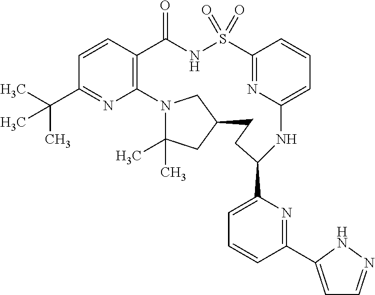 [SMILES: CC(C)(C)c1ccc2c(n1)N1C[C@@H](CC[C@H](c3cccc(-c4ccn[nH]4)n3)Nc3cccc(n3)S(=O)(=O)NC2=O)CC1(C)C] | 1.85 | 614.279 | 615.6 | LC method A |
| Compound 150, hydrochloride salt | 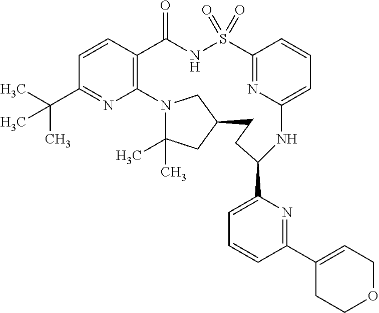 [SMILES: CC(C)(C)c1ccc2c(n1)N1C[C@@H](CC[C@H](c3cccc(C4=CCOCC4)n3)Nc3cccc(n3)S(=O)(=O)NC2=O)CC1(C)C] | 1.99 | 630.299 | 631.7 | LC method A |
| Compound 67, hydrochloride salt | 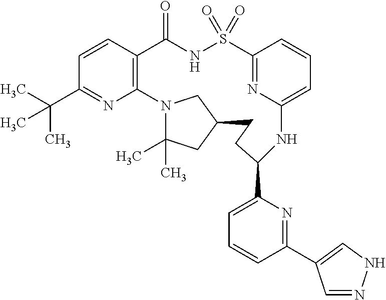 [SMILES: CC(C)(C)c1ccc2c(n1)N1C[C@@H](CC[C@H](c3cccc(-c4cn[nH]c4)n3)Nc3cccc(n3)S(=O)(=O)NC2=O)CC1(C)C] | 1.83 | 614.279 | 615.7 | LC method D |
| Compound 140 | 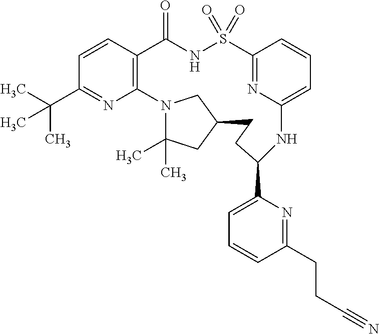 [SMILES: CC(C)(C)c1ccc2c(n1)N1C[C@@H](CC[C@H](c3cccc(CCC#N)n3)Nc3cccc(n3)S(=O)(=O)NC2=O)CC1(C)C] | 1.88 | 601.284 | 602.2 | LC method A |
| Compound 152, hydrochloride salt | 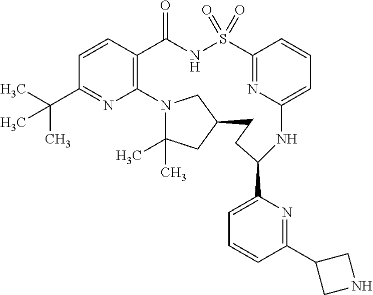 [SMILES: CC(C)(C)c1ccc2c(n1)N1C[C@@H](CC[C@H](c3cccc(C4CNC4)n3)Nc3cccc(n3)S(=O)(=O)NC2=O)CC1(C)C] | 1.64 | 603.299 | 604.7 | LC method A |
| Compound 148, hydrochloride salt | 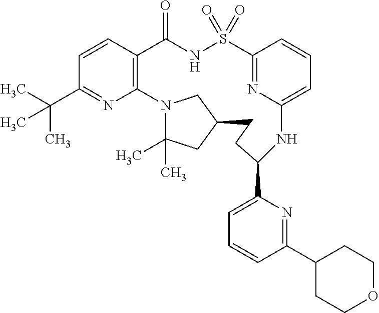 [SMILES: CC(C)(C)c1ccc2c(n1)N1C[C@@H](CC[C@H](c3cccc(C4CCOCC4)n3)Nc3cccc(n3)S(=O)(=O)NC2=O)CC1(C)C] | 1.77 | 632.314 | 633.7 | LC method A |
| Compound 104, hydrochloride salt | 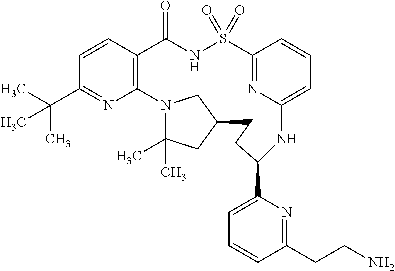 [SMILES: CC(C)(C)c1ccc2c(n1)N1C[C@@H](CC[C@H](c3cccc(CCN)n3)Nc3cccc(n3)S(=O)(=O)NC2=O)CC1(C)C] | 1.56 | 591.299 | 592.7 | LC method A |

| Compound | | |
|---|---|---|
| Number | Molecule | NMR |
| Compound 150, hydrochloride salt | 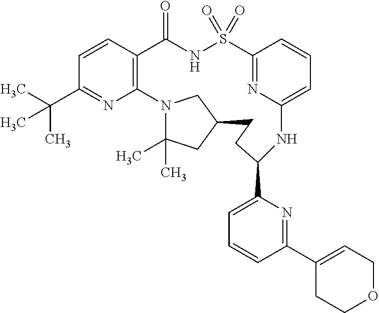 [SMILES: CC(C)(C)c1ccc2c(n1)N1C[C@@H](CC[C@H](c3cccc(C4=CCOCC4)n3)Nc3cccc(n3)S(=O)(=O)NC2=O)CC1(C)C] | ¹H NMR (400 MHz, DMSO-d₆) δ 12.46 (s, 1H), 7.74-7.58 (m, 4H), 7.35 (d, J = 7.8 Hz, 1H), 7.29 (d, J = 7.7 Hz, 1H), 7.10 (d, J = 7.1 Hz, 1H), 6.90 (d, J = 8.4 Hz, 1H), 6.82-6.78 (m, 1H), 6.65 (d, J = 8.0 Hz, 1H), 5.23 (dd, J = 11.6, 8.5 Hz, 1H), 4.26 (d, J = 3.0 Hz, 2H), 3.80 (t, J = 5.5 Hz, 2H), 3.16 (d, J = 2.4 Hz, 1H), 2.76 (t, J = 10.5 Hz, 1H), 2.58-2.54 (m, 1H), 2.21 (d, J = 14.3 Hz, 1H), 2.12 (s, 1H), 1.95-1.80 (m, 3H), 1.74 (s, 1H), 1.66 (s, 3H), 1.58 (t, J = 12.4 Hz, 1H), 1.51 (s, 3H), 1.45 (d, J = 10.7 Hz, 1H), 1.28 (s, 9H). |
| Compound 148, hydrochloride salt | 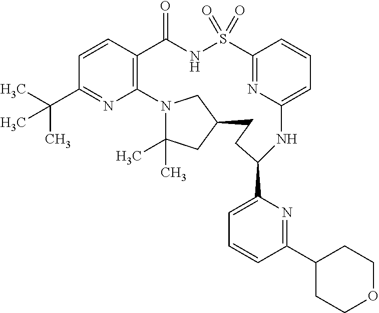 [SMILES: CC(C)(C)c1ccc2c(n1)N1C[C@@H](CC[C@H](c3cccc(C4CCOCC4)n3)Nc3cccc(n3)S(=O)(=O)NC2=O)CC1(C)C] | ¹H NMR (400 MHz, DMSO-d₆) δ 12.46 (s, 1H), 7.77-7.54 (m, 4H), 7.26 (s, 1H), 7.15 (s, 1H), 7.11 (d, J = 7.2 Hz, 1H), 6.90 (d, J = 8.4 Hz, 1H), 6.65 (d, J = 8.0 Hz, 1H), 5.21 (s, 1H), 4.00-3.88 (m, 2H), 3.56-3.44 (m, 2H), 3.17 (s, 1H), 2.92 (s, 1H), 2.75 (t, J = 10.5 Hz, 1H), 2.21 (s, 1H), 2.07 (d, J = 12.6 Hz, 1H), 1.93-1.82 (m, 2H), 1.76 (td, J = 10.6, 9.9, 6.1 Hz, 5H), 1.66 (s, 3H), 1.58 (t, J = 12.4 Hz, 1H), 1.51 (s, 3H), 1.46 (d, J = 13.5 Hz, 1H), 1.28 (s, 9H). |

### Example 21: Preparation of (14S,17R)-17-[6-(3-aminopropyl)pyridin-2-yl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 134

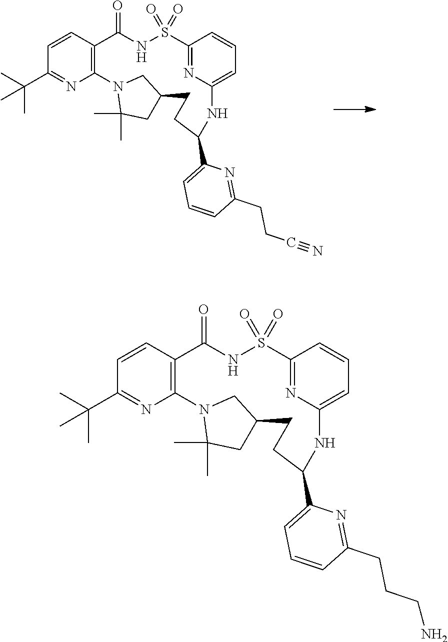 [SMILES: CC(C)(C)c1ccc2c(n1)N1C[C@@H](CC[C@H](c3cccc(CCC#N)n3)Nc3cccc(n3)S(=O)(=O)NC2=O)CC1(C)C ; CC(C)(C)c1ccc2c(n1)N1C[C@@H](CC[C@H](c3cccc(CCCN)n3)Nc3cccc(n3)S(=O)(=O)NC2=O)CC1(C)C]

In a 4 mL vial 3-{6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-2-yl}propanenitrile (24 mg, 0.0395 mmol) was dissolved in EtOH (2 mL) and purged with nitrogen for 5 minutes. To the mixture was added raney nickel (8 mg of 50% w/w, 0.0681 mmol) followed by a hydrogen balloon. The mixture was sealed and heated at 60° C. for 14 h. Cooled to room temperature. The hydrogen balloon was removed and the vessel was quickly degassed with nitrogen. Added 2 drops of conc. HCl, stirred 1 minute then filtered eluting with methanol. The filtrate was concentrated, dissolved in minimal DMSO then the mixture was filtered, and purified by reverse-phase preparative chromatography utilizing a C₁₈ column and a 1-70% gradient over 15 min of acetonitrile in water (+5 mM HCl) to afford (14S,17R)-17-[6-(3-aminopropyl)pyridin-2-yl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (17.7 mg, 68%). ¹H NMR (400 MHz, DMSO-d₆) δ 12.42 (s, 1H), 8.30 (s, 1H), 8.06 (s, 3H), 7.80 (s, 1H), 7.69 (d, J=7.8 Hz, 1H), 7.64 (d, J=8.0 Hz, 1H), 7.17 (d, J=7.2 Hz, 1H), 6.93 (d, J=8.4 Hz, 1H), 6.66 (d, J=8.0 Hz, 1H), 5.53 (s, 1H), 3.94 (s, 1H), 3.45 (s, 1H), 3.14 (s, 2H), 2.83 (q, J=6.2 Hz, 2H), 2.68 (t, J=10.3 Hz, 1H), 2.37 (s, 1H), 2.03 (p, J=7.4 Hz, 4H), 1.84 (dd, J=11.6, 5.1 Hz, 1H), 1.77 (d, J=14.3 Hz, 1H), 1.65 (s, 3H), 1.57 (t, J=12.4 Hz, 2H), 1.51 (s, 3H), 1.28 (s, 9H). ESI-MS m/z calc. 605.3148, found 606.5 (M+1)⁺; Retention time: 1.38 minutes (LC method A).

### Example 22: Preparation of (14S,17R)-8-tert-butyl-12,12-dimethyl-17-[6-(4-methylpiperazin-1-yl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione, Compound 195

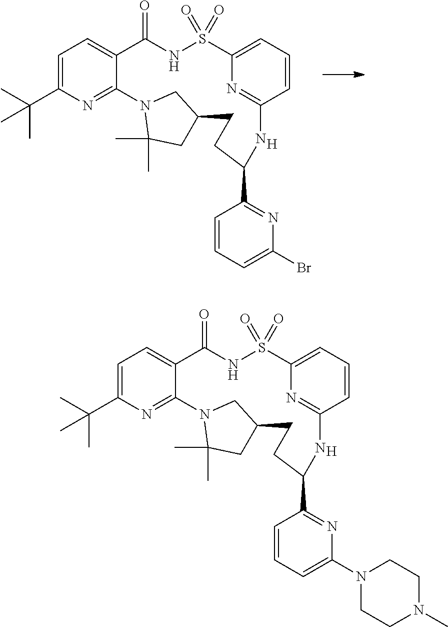

Combined (14S,17R)-17-(6-bromopyridin-2-yl)-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (16 mg, 0.02417 mmol) and 1-methylpiperazine (5 μL, 0.04834 mmol) in THF (300 μL) in a vial, capped, heated to 70° C. and stirred 30 minutes, then at 100° C. under microwave radiation for 30 minutes, then at 125° C. under microwave radiation for 30 minutes. Added 1-methylpiperazine (54 μL, 0.4834 mmol) and heated to 150° C. under microwave radiation for 8 h. The mixture was concentrated to remove THF then dissolved in DMSO, filtered and purified using a reverse phase HPLC-MS method using a Luna C₁₈ column (75×30 mm, 5 μm particle size) sold by Phenomenex (pn: 00C-4252-U0-AX), and a dual gradient run from 1-99% mobile phase B over 15.0 minutes (mobile phase A=H₂O (5 mM HCl), mobile phase B=acetonitrile, flow rate=50 mL/min, injection volume=950 μL and column temperature=25° C.). Co-eluted with a by-product. Isolated fractions containing product, dissolved in DMSO, filtered and purified using a reverse phase HPLC-MS method using a Luna C₁₈ column (75×30 mm, 5 μm particle size) sold by Phenomenex (pn: 00C-4252-U0-AX), and a dual gradient run from 25-75% mobile phase B over 15.0 minutes (mobile phase A=H₂O (5 mM HCl), mobile phase B=acetonitrile, flow rate=50 mL/min, injection volume=950 μL and column temperature=25° C.) giving as a white solid, (14S,17R)-8-tert-butyl-12,12-dimethyl-17-[6-(4-methylpiperazin-1-yl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione (hydrochloride salt) (4.77 mg, 28%). ESI-MS m/z calc. 646.3414, found 647.7 (M+1)⁺; Retention time: 1.67 minutes (LC method A).

The following is a list of amine reagents that are commercially available:
Morpholine
Piperidine
N′,AP-Dimethylethane-1,2-diamine

The compounds in the following tables were prepared in a manner analogous to that described above, using amine reagents given in the table above.

| | | LCMS | | | |
|---|---|---|---|---|---|
| | | Retention | | | |
| Compound | | Time | Exact | | LCMS |
| Number | Molecule | (min) | Mass | M + 1 | Method |
| Compound 191 | 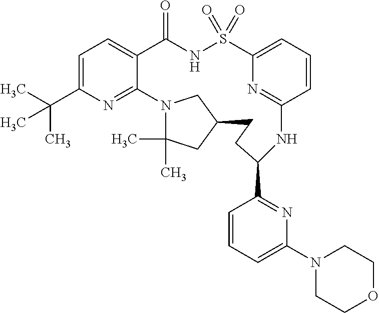 [SMILES: CC(C)(C)c1ccc2c(n1)N1C[C@@H](CC[C@H](c3cccc(N4CCOCC4)n3)Nc3cccc(n3)S(=O)(=O)NC2=O)CC1(C)C] | 1.79 | 633.31 | 634.7 | LC method A |
| Compound 174 | 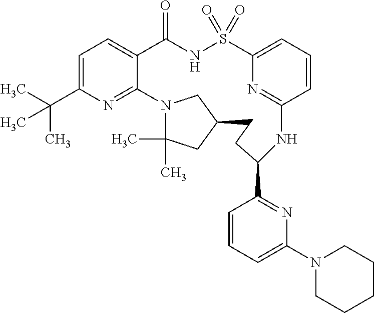 [SMILES: CC(C)(C)c1ccc2c(n1)N1C[C@@H](CC[C@H](c3cccc(N4CCCCC4)n3)Nc3cccc(n3)S(=O)(=O)NC2=O)CC1(C)C] | 1.78 | 631.33 | 632.7 | LC method A |
| Compound 189, hydrochloride salt | 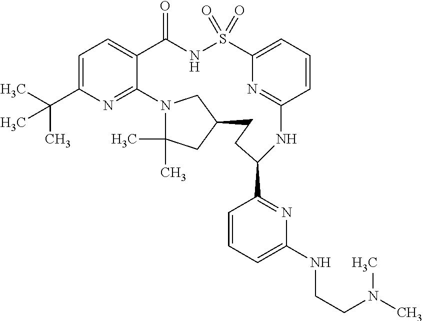 [SMILES: CN(C)CCNc1cccc([C@H]2CC[C@@H]3CN(c4nc(C(C)(C)C)ccc4C(=O)NS(=O)(=O)c4cccc(n4)N2)C(C)(C)C3)n1] | 1.47 | 634.341 | 635.7 | LC method A |

| Compound | | |
|---|---|---|
| Number | Molecule | NMR |
| Compound 174 | 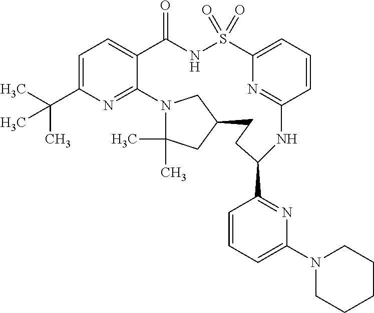 [SMILES: CC(C)(C)c1ccc2c(n1)N1C[C@@H](CC[C@H](c3cccc(N4CCCCC4)n3)Nc3cccc(n3)S(=O)(=O)NC2=O)CC1(C)C] | ¹H NMR (400 MHz, DMSO-d₆) δ 12.44 (s, 1H), 7.68-7.56 (m, 2H), 7.46 (d, J = 24.1 Hz, 2H), 7.08 (d, J = 12 Hz, 1H), 6.88 (d, J = 8.4 Hz, 1H), 6.64 (d, J = 8.0 Hz, 3H), 5.09 (s, 1H), 3.50 (s, 4H), 3.17 (s, 1H), 2.74 (s, 1H), 2.20 (s, 2H), 2.04 (d, J = 12.7 Hz, 1H), 1.90-1.80 (m, 2H), 1.73 (d, J = 14.2 Hz, 1H), 1.65 (s, 3H), 1.55 (d, J = 10.3 Hz, 6H), 1.50 (s, 3H), 1.45 (d, J = 13.5 Hz, 1H), 1.28 (s, 9H). |

### Example 23: Preparation of 6-[(14S,17R)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridine-2-carbonitrile, Compound 211

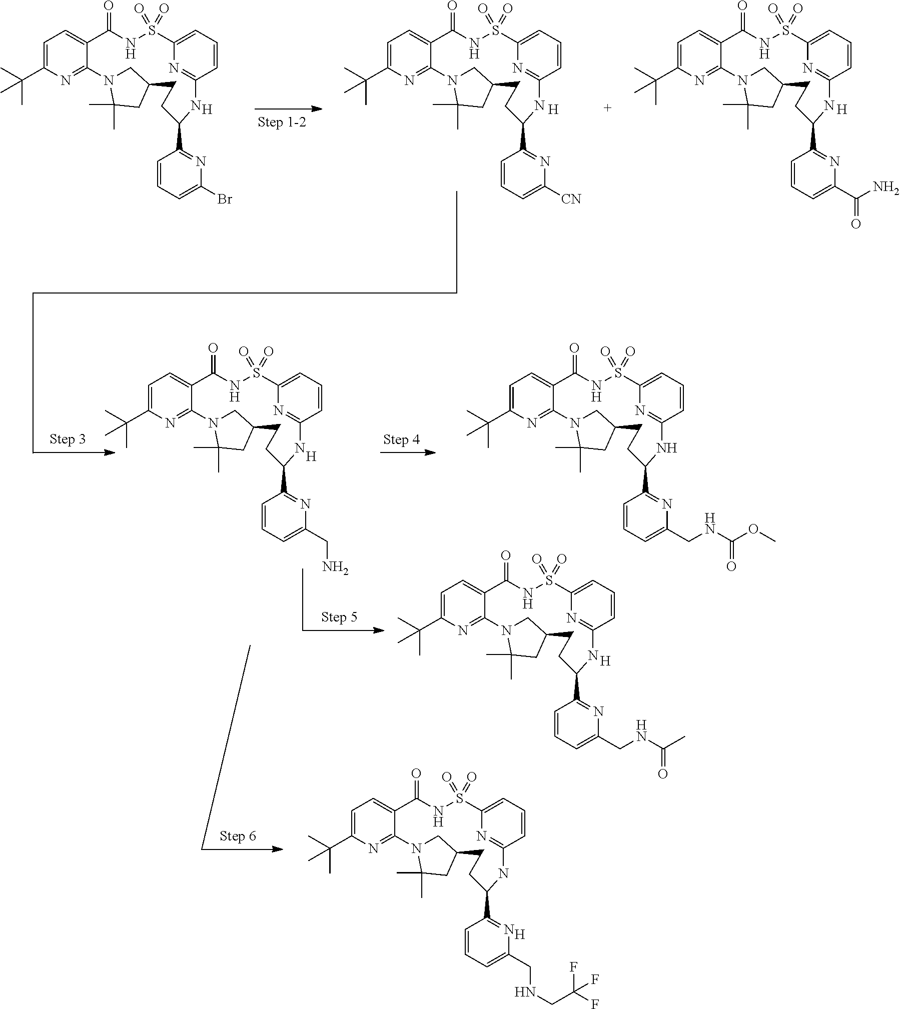

### Step 1: 6-[(14S,17R)-8-tert-Butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridine-2-carbonitrile, Compound 211

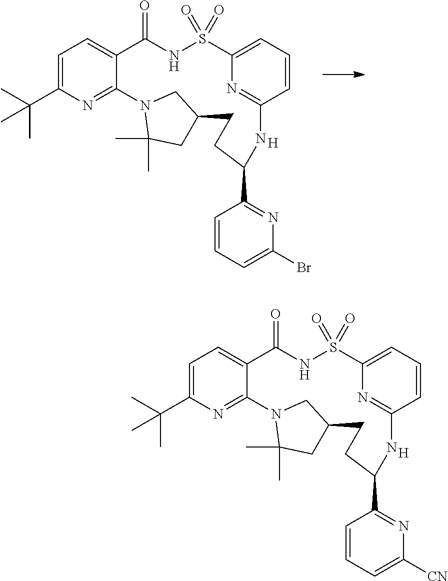

To (14S,17R)-17-(6-bromopyridin-2-yl)-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (24 mg, 0.0362 mmol) in DMF (227 μL) was added dicyanozinc (2.8 μL, 0.0435 mmol) followed by Pd(PPh₃)₄ (5 mg, 0.00435 mmol). The reaction mixture was allowed to stir under nitrogen at 150° C. for 30 minutes. Filtered and purified using a reverse phase HPLC-MS method using a Luna C₁₈ column (75×30 mm, 5 μm particle size) sold by Phenomenex (pn: 00C-4252-U0-AX), and a dual gradient run from 30-99% mobile phase B over 15.0 minutes (mobile phase A=H₂O (5 mM HCl), mobile phase B=acetonitrile, flow rate=50 mL/min, injection volume=950 μL and column temperature=25° C.) giving as a white solid, 6-[(14S,17R)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridine-2-carbonitrile (15.6 mg, 75%). ESI-MS m/z calc. 573.2522, found 574.6 (M+1)⁺; Retention time: 2.12 minutes (LC method A).

### Step 2: 6-[(14S,17R)-8-tert-Butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridine-2-carbonitrile, Compound 211, and 6-[(14S,17R)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridine-2-carboxamide, Compound 70

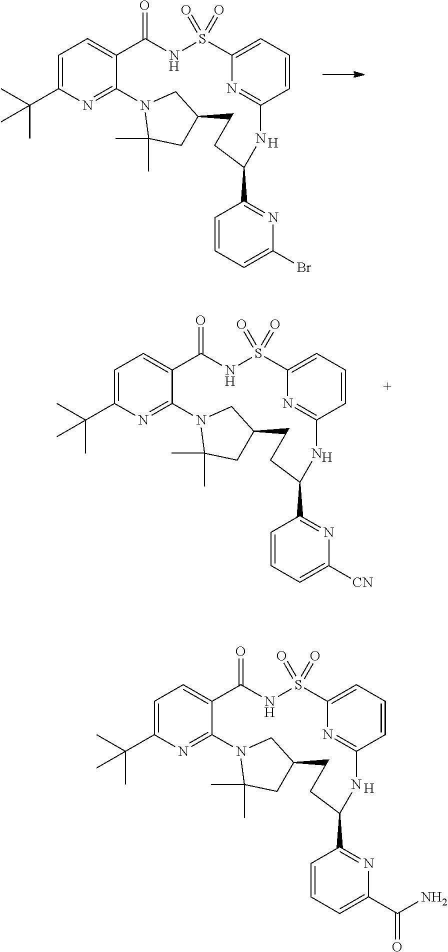

To (14S,17R)-17-(6-bromopyridin-2-yl)-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (107.5 mg, 0.1713 mmol) in DMF (1 mL) was added dicyanozinc (13 μL, 0.206 mmol) followed by Pd(PPh₃)₄ (24 mg, 0.0206 mmol). The reaction mixture was allowed to stir under nitrogen at 150° C. for 2 h. Filtered and purified using a reverse phase HPLC-MS method using a Luna C₁₈ column (75×30 mm, 5 μm particle size) sold by Phenomenex (pn: 00C-4252-U0-AX), and a dual gradient run from 30-99% mobile phase B over 15.0 minutes (mobile phase A=H₂O (5 mM HCl), mobile phase B=acetonitrile, flow rate=50 mL/min, injection volume=950 μL and column temperature=25° C.) giving as a white solid, 6-[(14S,17R)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridine-2-carbonitrile (60.5 mg, 62%). ESI-MS m/z calc. 573.2522, found 574.6 (M+1)⁺, Retention time: 0.81 minutes (LC method D).

A side product was isolated as a white solid, 6-[(14S,17R)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridine-2-carboxamide (8.5 mg, 8%) (Compound 70), ESI-MS m/z calc. 591.26276, found 592.6 (M+1)⁺; Retention time: 1.95 minutes (LC method A).

### Step 3: (14S,17R)-17-[6-(Aminomethyl)pyridin-2-yl]-8-tert-butyl-12,12-dimethyl-R⁶-thia-3,9,11,18,23-pentaazatetracyclo[17.3.1.111,14.05,10]tetracosa-1(22),5,7,9,19(23),20-hexaene-2,2,4-trione, Compound 203

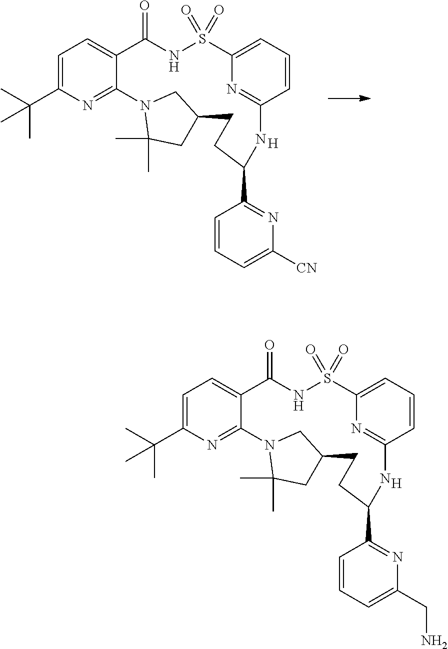

6-[(14S,17R)-8-tert-Butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridine-2-carbonitrile (12.9 mg, 0.0225 mmol) was dissolved in ethanol (1 mL) and wet Raney nickel (1.3 mg, 0.0229 mmol) was added under nitrogen atmosphere. Nitrogen was bubbled through the stirring suspension for 2 minutes followed by bubbling hydrogen for 2 minutes. The mixture was outfitted with a hydrogen balloon and stirred overnight. The hydrogen balloon was removed and the vessel was quickly degassed with nitrogen. Added 2 drops of conc. HCl, stirred 1 minute then filtered eluting with methanol. The filtrate was concentrated, dissolved in minimal DMSO, filtered and purified using a reverse phase HPLC-MS method using a Luna C₁₈ column (75×30 mm, 5 μm particle size) sold by Phenomenex (pn: 00C-4252-U0-AX), and a dual gradient run from 30-99% mobile phase B over 15.0 minutes (mobile phase A=H₂O (5 mM HCl), mobile phase B=acetonitrile, flow rate=50 mL/min, injection volume=950 μL and column temperature=25° C.) giving (14S,17R)-17-[6-(aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (hydrochloride salt) (5.1 mg, 37%) as a white solid. ¹H NMR (500 MHz, DMSO-d₆) δ 12.55 (s, 1H), 8.53 (s, 3H), 7.81 (t, J=7.8 Hz, 2H), 7.69-7.60 (m, 2H), 7.41 (d, J=7.8 Hz, 1H), 7.36 (d, J=7.7 Hz, 1H), 7.12 (d, J 7.2 Hz, 1H), 6.99 (d, J=8.5 Hz, 1H), 6.65 (d, J=8.0 Hz, 1H), 5.26 (t, J=10.1 Hz, 1H), 4.17 (d, J=5.8 Hz, 2H), 3.22 (t, J=8.7 Hz, 1H), 2.79 (t, J=10.4 Hz, 1H), 2.30 (s, 1H), 2.13 (t, J 12.9 Hz, 1H), 1.86 (dd, J 12.2, 5.4 Hz, 2H), 1.74 (d, J 12.7 Hz, 1H), 1.67 (s, 3H), 1.60 (d, J=12.5 Hz, 1H), 1.51 (s, 3H), 1.48 (s, 1H), 1.28 (s, 9H). ESI-MS m/z calc. 577.2835, found 578.6 (M+1)⁺; Retention time: 1.61 minutes (LC method A).

### Step 4: Methyl N-({6-[(14S,17R)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-2-yl}methyl)carbamate, Compound 101

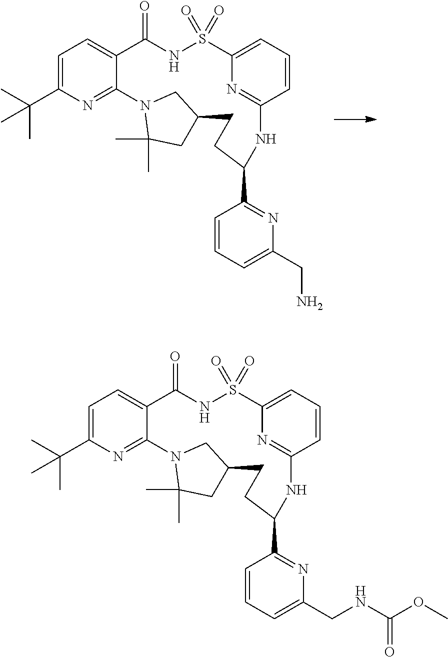 [SMILES: CC(C)(C)c1ccc2c(n1)N1C[C@@H](CC[C@H](c3cccc(CN)n3)Nc3cccc(n3)S(=O)(=O)NC2=O)CC1(C)C ; COC(=O)NCc1cccc([C@H]2CC[C@@H]3CN(c4nc(C(C)(C)C)ccc4C(=O)NS(=O)(=O)c4cccc(n4)N2)C(C)(C)C3)n1]

Dissolved (14S,17R)-17-[6-(aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (22.5 mg, 0.03894 mmol) in DCM (2 mL) then added TEA (22 μL, 0.156 mmol) followed by methyl chloroformate (3 μL, 0.0389 mmol) and the resulting mixture was stirred at rt for 13 min, then added methyl chloroformate (3 μL, 0.03896 mmol) and TEA (21.72 μL, 0.1558 mmol), stirred for 5 min, heated to 60° C., stirred for 3 min then cooled to rt and added methyl chloroformate (9 μL, 0.1168 mmol) and stirred for 5 min, then added TEA (21.72 μL, 0.1558 mmol) and methyl chloroformate (15 μL, 0.1947 mmol) and stirred for 10 min. Cooled the rm to 0° C. and added TEA (54 μL, 0.3894 mmol) followed by methyl chloroformate (60 μL, 0.7787 mmol) and continued stirring at 0° C. for 3 h. Diluted with 1N HCl, extracted with DCM, then extracted with EtOAc. Combined the organic phases, dried (MgSO₄), filtered and concentrated to a residue which was dissolved in DMSO, filtered and purified using a reverse phase HPLC-MS method using a Luna C₁₈ column (75×30 mm, 5 μm particle size) sold by Phenomenex (pn: 00C-4252-U0-AX), and a dual gradient run from 30-99% mobile phase B over 15.0 minutes (mobile phase A=H₂O (5 mM HCl), mobile phase B=acetonitrile, flow rate=50 mL/min, injection volume=950 μL and column temperature=25° C.) giving as a white solid, methyl N-({6-[(14S,17R)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-2-yl}methyl)carbamate (1.29 mg, 4%). ESI-MS m/z calc. 635.289, found 636.7 (M+1)⁺; Retention time: 1.76 minutes (LC method A).

### Step 5: N-({6-[(14S,17R)-8-tert-Butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-2-yl}methyl)acetamide, Compound 100

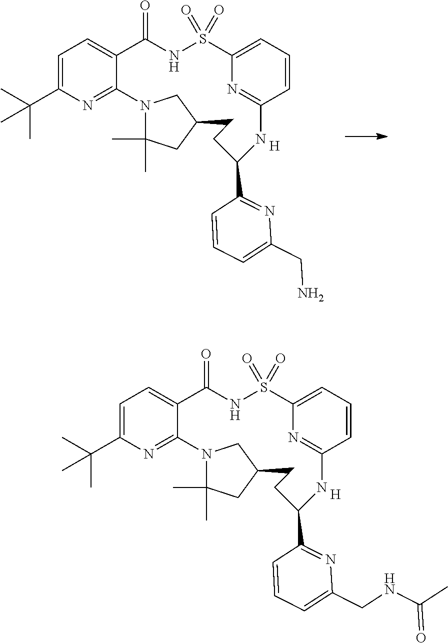

To a stirring solution of (14S,17R)-17-[6-(aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (22.5 mg, 0.03894 mmol) in DCM (22 mL) at 0° C. was added TEA (109 μL, 0.7792 mmol) followed by acetyl chloride (17 μL, 0.2336 mmol) and the resulting mixture was stirred at 0° C. for 3 h. Diluted with 1N HCl, extracted with DCM, then extracted with EtOAc. Combined the organic phases, dried (MgSO₄), filtered and concentrated to a residue which was dissolved in DMSO, filtered and purified using a reverse phase HPLC-MS method using a Luna C₁₈ column (75×30 mm, 5 μm particle size) sold by Phenomenex (pn: 00C-4252-U0-AX), and a dual gradient run from 30-99% mobile phase B over 15.0 minutes (mobile phase A=H₂O (5 mM HCl), mobile phase B=acetonitrile, flow rate=50 mL/min, injection volume=950 μL and column temperature=25° C.) giving as a white solid, N-({6-[(14S,17R)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-2-yl}methyl)acetamide (5.5 mg, 23%). ¹H NMR (400 MHz, DMSO-d₆) δ 12.49 (s, 1H), 8.48 (t, J=5.8 Hz, 1H), 7.81 (s, 1H), 7.72 (d, J=9.0 Hz, 1H), 7.69-7.64 (m, 1H), 7.61 (d, J=8.0 Hz, 1H), 7.39 (d, J=7.7 Hz, 1H), 7.20 (d, J=7.6 Hz, 1H), 7.12 (d, J=7.2 Hz, 1H), 6.91 (d, J=8.5 Hz, 1H), 6.65 (d, J=8.0 Hz, 1H), 5.25 (t, J=10.8 Hz, 1H), 4.37 (dd, J=15.5, 5.9 Hz, 2H), 3.20 (dd, J=10.3, 6.8 Hz, 1H), 2.74 (t, J=10.4 Hz, 1H), 2.24 (s, 1H), 2.09-1.97 (m, 1H), 1.91 (s, 3H), 1.89-1.80 (m, 2H), 1.74 (dd, J=14.3, 5.6 Hz, 1H), 1.66 (s, 3H), 1.58 (t, J=12.4 Hz, 1H), 1.51 (s, 3H), 1.49-1.40 (m, 1H), 1.28 (s, 9H). ESI-MS m/z calc. 619.29407, found 620.7 (M+1)⁺; Retention time: 1.65 minutes (LC method A).

### Step 6: (14S,17R)-8-tert-Butyl-12,12-dimethyl-17-(6-{[(2,2,2-trifluoroethyl)amino]methyl}pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 50

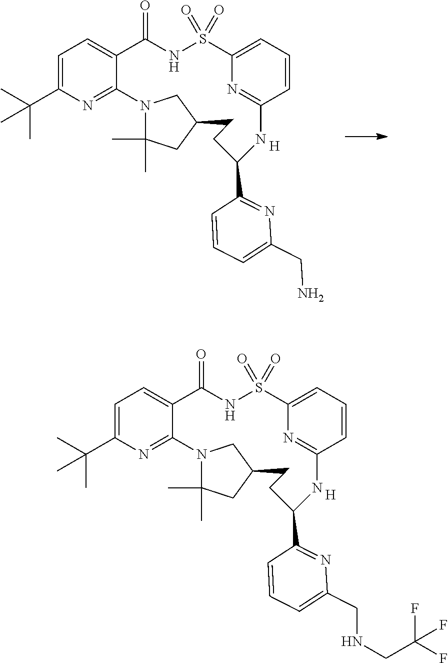

Combined (14S,17R)-17-[6-(aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (26 mg, 0.04500 mmol), DIEA (27 μL, 0.157 mmol) and DMF (1 mL) in a vial, cooled to 0° C. under a nitrogen atmosphere and slowly added a pre-cooled (0° C.) solution of 2,2,2-trifluoroethyl trichloromethanesulfonate (9 μL, 0.0540 mmol) in DMF (1 mL) and on completion of addition, stirred for 5 min at 0° C. then warmed to rt and stirred for 4 h. Added 2,2,2-trifluoroethyl trichloromethanesulfonate (15.2 mg, 0.05400 mmol) and DIEA (27 μL, 0.157 mmol) and heated to 75° C. and stirred 24 h. Filtered through a small plug of sand then purified using a reverse phase HPLC-MS method using a Luna C₁₈ column (75×30 mm, 5 μm particle size) sold by Phenomenex (pn: 00C-4252-U0-AX), and a dual gradient run from 30-99% mobile phase B over 15.0 minutes (mobile phase A=H₂O (5 mM HCl), mobile phase B=acetonitrile, flow rate=50 mL/min, injection volume=950 μL and column temperature=25° C.) giving as an off-white solid, (14S,17R)-8-tert-butyl-12,12-dimethyl-17-(6-{[(2,2,2-trifluoroethyl)amino]methyl}pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (0.86 mg, 3%). ESI-MS m/z calc. 659.28656, found 660.7 (M+1)⁺; Retention time: 1.78 minutes (LC method A).

### Example 24: Preparation of analogs of (14S,17S)-17-(6-bromopyridin-2-yl)-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, Compound 214

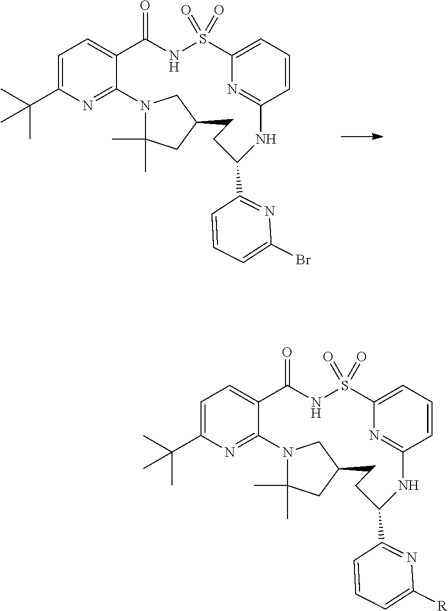

The compounds in the following tables were prepared in a manner analogous to that described above using (14S,17S)-17-(6-bromopyridin-2-yl)-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, Compound 214 as a starting material.

| Compound | | |
|---|---|---|
| Number | Molecule | NMR |
| Compound 149, hydrochloride salt | 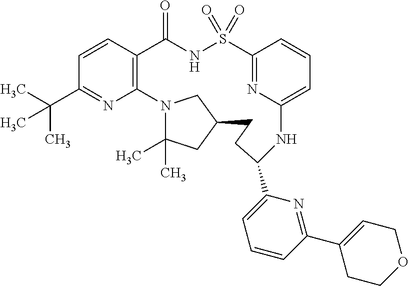 [SMILES: CC(C)(C)c1ccc2c(n1)N1C[C@@H](CC[C@@H](c3cccc(C4=CCOCC4)n3)Nc3cccc(n3)S(=O)(=O)NC2=O)CC1(C)C] | ¹H NMR (400 MHz, DMSO-d₆) δ 12.25 (s, 1H), 7.73 (t, J = 7.8 Hz, 1H), 7.67-7.56 (m, 2H), 7.49 (d, J = 7.1 Hz, 1H), 7.40 (d, J = 7.8 Hz, 2H), 7.21 (d, J = 7.2 Hz, 1H), 6.88 (d, J = 8.5 Hz, 1H), 6.81 (s, 1H), 6.63 (d, J = 7.9 Hz, 1H), 4.96 (s, 1H), 4.27 (q, J = 2.9 Hz, 2H), 3.82 (t, J = 5.5 Hz, 2H), 3.11 (s, 2H), 2.55 (d, J = 8.3 Hz, 2H), 2.39-2.14 (m, 4H), 1.86 (dd, J = 12.0, 6.0 Hz, 1H), 1.77-1.68 (m, 1H), 1.61 (s, 3H), 1.58 (s, 1H), 1.53 (s, 3H), 1.28 (s, 9H). |
| Compound 133, hydrochloride salt | 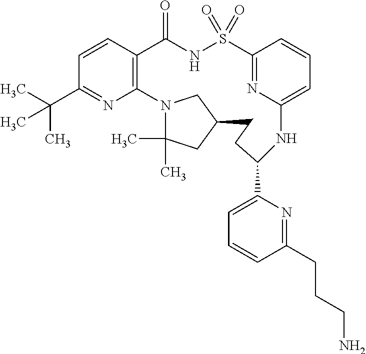 [SMILES: CC(C)(C)c1ccc2c(n1)N1C[C@@H](CC[C@@H](c3cccc(CCCN)n3)Nc3cccc(n3)S(=O)(=O)NC2=O)CC1(C)C] | ¹H NMR (400 MHz, DMSO-d₆) δ 12.47 (s, 1H), 8.26 (s, 1H), 8.08 (s, 3H), 8.02-7.88 (m, 2H), 7.72 (d, J = 8.0 Hz, 1H), 7.47 (d, J = 7.9 Hz, 1H), 7.33 (d, J = 7.3 Hz, 1H), 6.94 (d, J = 8.5 Hz, 1H), 6.63 (d, J = 7.9 Hz, 1H), 5.16 (s, 1H), 3.21 (s, 1H), 3.16-3.08 (m, 2H), 2.83 (td, J = 11.8, 5.1 Hz, 3H), 2.28 (dd, J = 16.9, 7.1 Hz, 1H), 2.04 (p, J = 7.5, 6.8 Hz, 3H), 1.86 (dd, J = 11.8, 5.7 Hz, 1H), 1.76 (dt, J = 13.8, 8.1 Hz, 1H), 1.55 (d, J = 8.6 Hz, 6H), 1.49 (d, J = 11.5 Hz, 1H), 1.27 (s, 10H), 1.18 (dd, J = 16.3, 8.0 Hz, 1H). |
| Compound 147, hydrochloride salt | 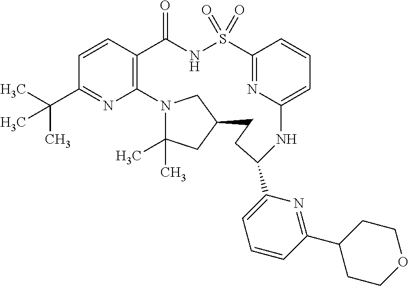 [SMILES: CC(C)(C)c1ccc2c(n1)N1C[C@@H](CC[C@@H](c3cccc(C4CCOCC4)n3)Nc3cccc(n3)S(=O)(=O)NC2=O)CC1(C)C] | ¹H NMR (400 MHz, DMSO-d₆) δ 12.28 (s, 2H), 7.70 (s, 1H), 7.63 (q, J = 7.2, 6.4 Hz, 2H), 7.48 (s, 1H), 7.39 (s, 1H), 7.22 (d, J = 7.5 Hz, 2H), 6.88 (d, J = 8.5 Hz, 1H), 6.63 (d, J = 7.9 Hz, 1H), 4.92 (s, 1H), 3.95 (dt, J = 11.3, 3.2 Hz, 2H), 3.43 (s, 2H), 3.11 (s, 2H), 2.94 (s, 1H), 2.30 (s, 1H), 2.17 (s, 1H), 1.85 (dd, J = 11.8, 5.9 Hz, 1H), 1.79 (h, J = 4.1, 3.3 Hz, 4H), 1.75-1.66 (m, 1H), 1.60 (s, 3H), 1.56 (s, 1H), 1.53 (s, 3H), 1.51 (s, 1H), 1.27 (s, 9H). |
| Compound 131, hydrochloride salt | 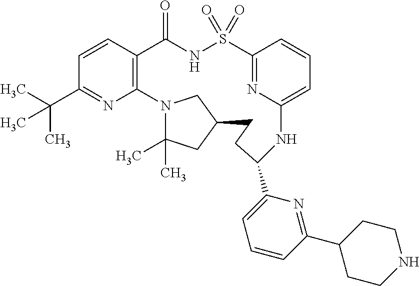 [SMILES: CC(C)(C)c1ccc2c(n1)N1C[C@@H](CC[C@@H](c3cccc(C4CCNCC4)n3)Nc3cccc(n3)S(=O)(=O)NC2=O)CC1(C)C] | ¹H NMR (400 MHz, DMSO-d₆) δ 12.29 (s, 1H), 8.91 (s, 1H), 8.63 (d, J = 8.5 Hz, 1H), 7.78 (s, 1H), 7.65 (t, J = 7.9 Hz, 2H), 7.48 (d, J = 7.9 Hz, 2H), 7.22 (dq, J = 14.8, 7.8, 7.1 Hz, 2H), 6.89 (d, J = 8.5 Hz, 1H), 6.63 (d, J = 8.0 Hz, 1H), 4.93 (s, 1H), 3.37 (d, J = 12.5 Hz, 3H), 3.08-2.94 (m, 4H), 2.31 (dd, J = 12.2, 5.2 Hz, 1H), 2.17 (d, J = 8.7 Hz, 1H), 2.06 (d, J = 13.5 Hz, 2H), 2.01-1.89 (m, 2H), 1.86 (dd, J = 11.9, 6.0 Hz, 1H), 1.78- 1.65 (m, 2H), 1.60 (s, 3H), 1.55 (d, J = 3.1 Hz, 1H), 1.52 (s, 3H), 1.27 (s, 9H). |
| Compound 173 | 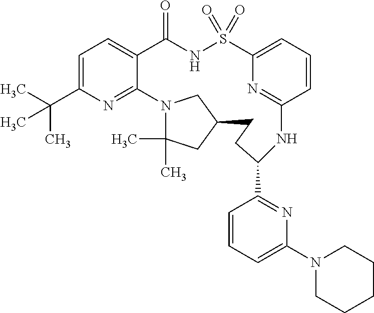 [SMILES: CC(C)(C)c1ccc2c(n1)N1C[C@@H](CC[C@@H](c3cccc(N4CCCCC4)n3)Nc3cccc(n3)S(=O)(=O)NC2=O)CC1(C)C] | ¹H NMR (400 MHz, DMSO-d₆) δ 12.27 (s, 1H), 7.64 (t, J = 7.9 Hz, 1H), 7.49 (s, 3H), 7.22 (s, 1H), 6.87 (d, J = 8.4 Hz, 3H), 6.63 (d, J = 8.0 Hz, 1H), 4.92 (s, 1H), 3.57 (s, 4H), 3.10 (s, 2H), 2.36-2.25 (m, 1H), 2.11 (s, 2H), 1.86 (dd, J = 11.9, 5.9 Hz, 1H), 1.78- 1.67 (m, 1H), 1.59 (d, J = 12.2 Hz, 10H), 1.53 (s, 3H), 1.27 (s, 9H), 1.23 (s, 1H). |
| Compound 202, hydrochloride salt | 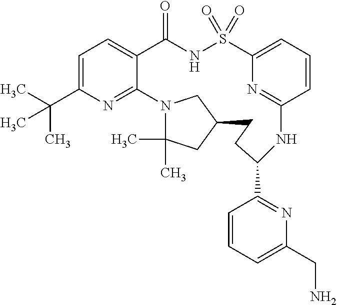 [SMILES: CC(C)(C)c1ccc2c(n1)N1C[C@@H](CC[C@@H](c3cccc(CN)n3)Nc3cccc(n3)S(=O)(=O)NC2=O)CC1(C)C] | ¹H NMR (500 MHz, DMSO-d₆) δ 12.25 (s, 1H), 8.60 (s, 3H), 7.91 (s, 1H), 7.85 (t, J = 7.7 Hz, 1H), 7.65 (t, J = 7.8 Hz, 1H), 7.51 (s, 2H), 7.40 (d, J = 7.7 Hz, 1H), 7.24 (s, 1H), 7.05 (d, J = 8.4 Hz, 1H), 6.66 (d, J = 8.2 Hz, 1H), 5.04 (s, 1H), 4.22 (d, J = 6.6 Hz, 2H), 3.38-3.19 (m, 1H), 3.10 (s, 1H), 2.30 (s, 1H), 2.12 (s, 1H), 1.86 (dd, J = 11.9, 6.0 Hz, 1H), 1.72 (s, 1H), 1.64 (s, 3H), 1.57 (d, J = 11.4 Hz, 2H), 1.51 (s, 3H), 1.28 (s, 9H), 1.24 (s, 1H). |

| | | LCMS | | | |
|---|---|---|---|---|---|
| | | Retention | | | |
| Compound | | Time | Exact | | LCMS |
| Number | Molecule | (min) | Mass | M + 1 | Method |
| Compound 149, hydrochloride salt | 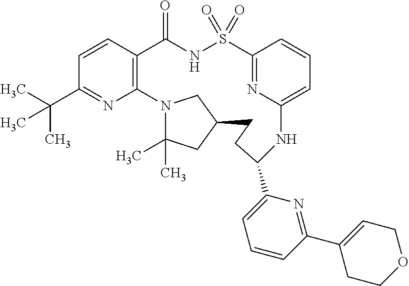 [SMILES: CC(C)(C)c1ccc2c(n1)N1C[C@@H](CC[C@@H](c3cccc(C4=CCOCC4)n3)Nc3cccc(n3)S(=O)(=O)NC2=O)CC1(C)C] | 2 | 630.299 | 631.7 | LC method A |
| Compound 151, hydrochloride salt | 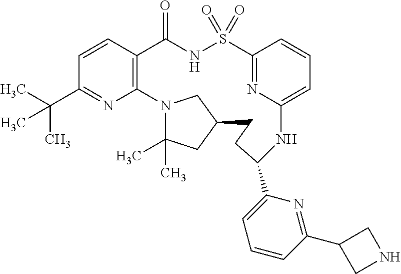 [SMILES: CC(C)(C)c1ccc2c(n1)N1C[C@@H](CC[C@@H](c3cccc(C4CNC4)n3)Nc3cccc(n3)S(=O)(=O)NC2=O)CC1(C)C] | 1.55 | 603.299 | 604.6 | LC method A |
| Compound 133, hydrochloride salt | 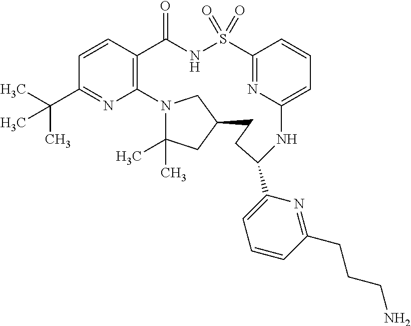 [SMILES: CC(C)(C)c1ccc2c(n1)N1C[C@@H](CC[C@@H](c3cccc(CCCN)n3)Nc3cccc(n3)S(=O)(=O)NC2=O)CC1(C)C] | 1.26 | 605.315 | 606.5 | LC method A |
| Compound 139 | 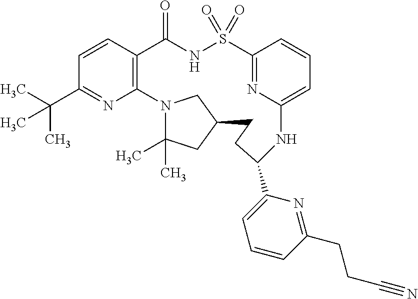 [SMILES: CC(C)(C)c1ccc2c(n1)N1C[C@@H](CC[C@@H](c3cccc(CCC#N)n3)Nc3cccc(n3)S(=O)(=O)NC2=O)CC1(C)C] | 1.8 | 601.284 | 602.2 | LC method A |
| Compound 147, hydrochloride salt | 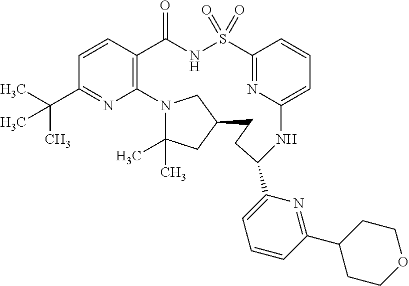 [SMILES: CC(C)(C)c1ccc2c(n1)N1C[C@@H](CC[C@@H](c3cccc(C4CCOCC4)n3)Nc3cccc(n3)S(=O)(=O)NC2=O)CC1(C)C] | 1.71 | 632.314 | 633.7 | LC method A |
| Compound 131, hydrochloride salt | 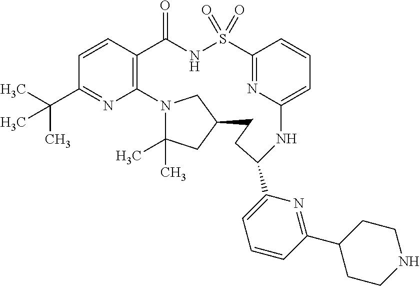 [SMILES: CC(C)(C)c1ccc2c(n1)N1C[C@@H](CC[C@@H](c3cccc(C4CCNCC4)n3)Nc3cccc(n3)S(=O)(=O)NC2=O)CC1(C)C] | 1.39 | 631.33 | 632.7 | LC method A |
| Compound 141, hydrochloride salt | 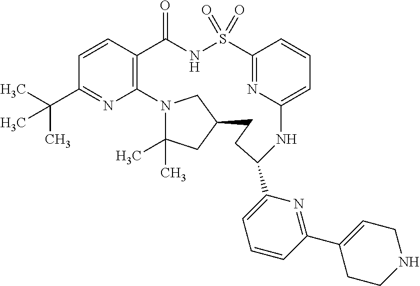 [SMILES: CC(C)(C)c1ccc2c(n1)N1C[C@@H](CC[C@@H](c3cccc(C4=CCNCC4)n3)Nc3cccc(n3)S(=O)(=O)NC2=O)CC1(C)C] | 1.67 | 629.315 | 630.7 | LC method A |
| Compound 194, hydrochloride salt | 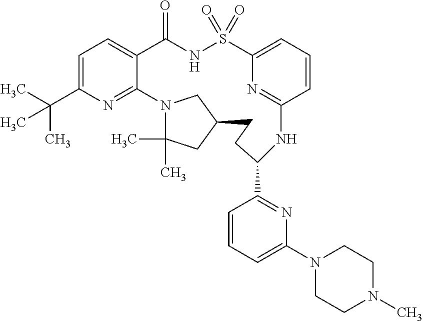 [SMILES: CN1CCN(c2cccc([C@@H]3CC[C@@H]4CN(c5nc(C(C)(C)C)ccc5C(=O)NS(=O)(=O)c5cccc(n5)N3)C(C)(C)C4)n2)CC1] | 1.6 | 646.341 | 647.7 | LC method A |
| Compound 190 | 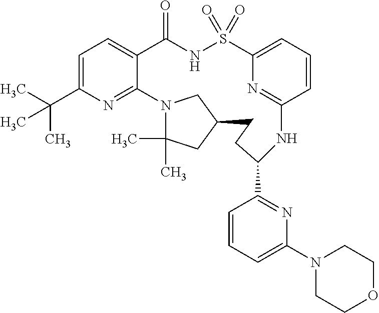 [SMILES: CC(C)(C)c1ccc2c(n1)N1C[C@@H](CC[C@@H](c3cccc(N4CCOCC4)n3)Nc3cccc(n3)S(=O)(=O)NC2=O)CC1(C)C] | 1.81 | 633.31 | 634.7 | LC method A |
| Compound 173 | 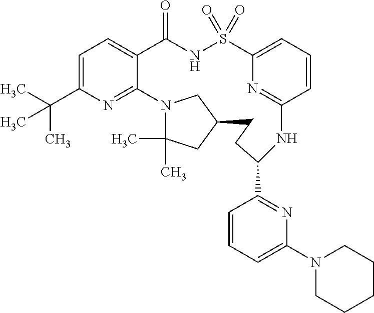 [SMILES: CC(C)(C)c1ccc2c(n1)N1C[C@@H](CC[C@@H](c3cccc(N4CCCCC4)n3)Nc3cccc(n3)S(=O)(=O)NC2=O)CC1(C)C] | 1.79 | 631.33 | 632.7 | LC method A |
| Compound 188, hydrochloride salt | 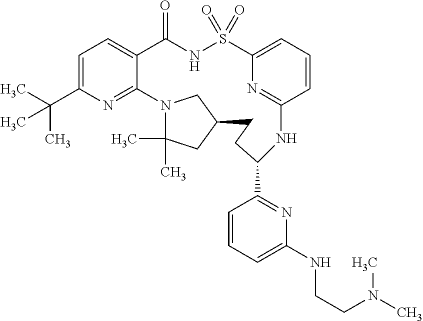 [SMILES: CN(C)CCNc1cccc([C@@H]2CC[C@@H]3CN(c4nc(C(C)(C)C)ccc4C(=O)NS(=O)(=O)c4cccc(n4)N2)C(C)(C)C3)n1] | 1.47 | 634.341 | 635.7 | LC method A |
| Compound 202, hydrochloride salt | 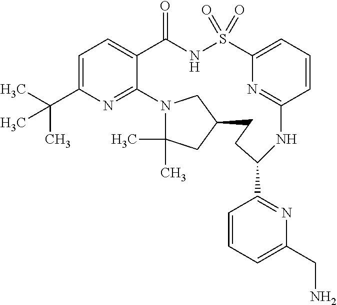 [SMILES: CC(C)(C)c1ccc2c(n1)N1C[C@@H](CC[C@@H](c3cccc(CN)n3)Nc3cccc(n3)S(=O)(=O)NC2=O)CC1(C)C] | 1.53 | 577.284 | 578.6 | LC method A |
| Compound 210 | 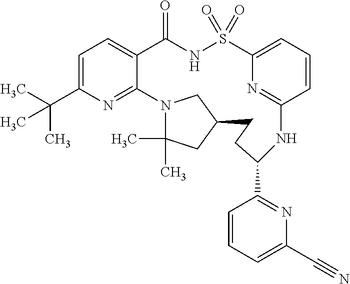 [SMILES: CC(C)(C)c1ccc2c(n1)N1C[C@@H](CC[C@@H](c3cccc(C#N)n3)Nc3cccc(n3)S(=O)(=O)NC2=O)CC1(C)C] | 2.12 | 573.252 | 574.6 | LC method A |

### Example 25: Preparation of (14S)-8-tert-butyl-12,12-dimethyl-17-[5-(4-methylpiperazin-1-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione, Compound 201

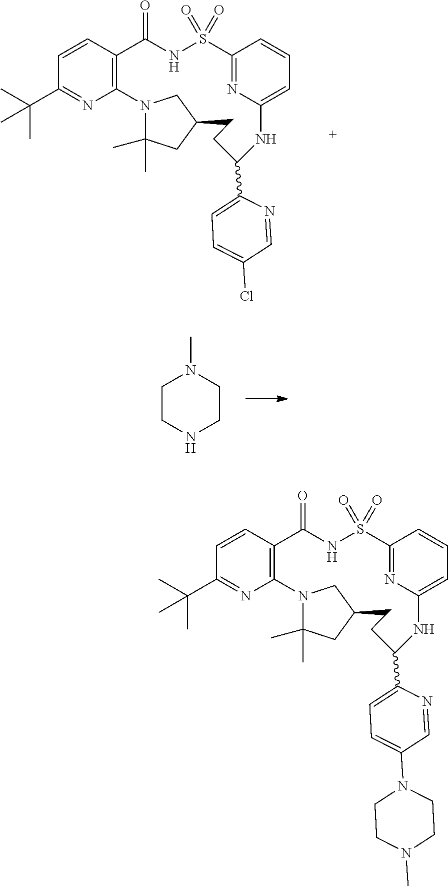

In a microwave vial (14S)-8-tert-butyl-17-(5-chloropyridin-2-yl)-12,12-dimethyl-2λ⁶-thia-3,9,11,18,23-pentanzatetracyclo[17.3.1.111,14.05,10]tetracosa-1(23),5(10),6,8,19,21-hexaene-2,2,4-trione (diastereomer 1, Compound 209, 20 mg, 0.0343 mmol) was dissolved in 1,4-dioxane (750 μL) and to the mixture was added potassium tert-butoxide (6 mg, 0.05347 mmol) followed by 1-methylpiperazine (5 mg, 0.0499 mmol) and (1,3-Bis(2,6-diisopropylphenyl)imidazolidene) (3-chloropyridyl) palladium(II) dichloride (PEPPSI-SIPr) (3 mg, 0.0044 mmol). The mixture was purged with nitrogen, sealed and heated to 100° C. for 4 h. Cooled to room temperature and the mixture was filtered, and purified by reverse-phase preparative chromatography utilizing a C₁₈ column and a gradient of 1-70% j over 15 min of acetonitrile in water (+5 mM HCl) to afford (14S)-8-tert-butyl-12,12-dimethyl-17-[5-(4-methylpiperazin-1-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (4.6 mg, 19%). ESI-MS m/z calc. 646.3414, found 647.3 (M+1)⁺; Retention time: 1.28 minutes (LC method A).

### Example 26: Preparation of (14S)-8-tert-butyl-12,12-dimethyl-17-[5-(2-methylpropyl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione, Compound 181

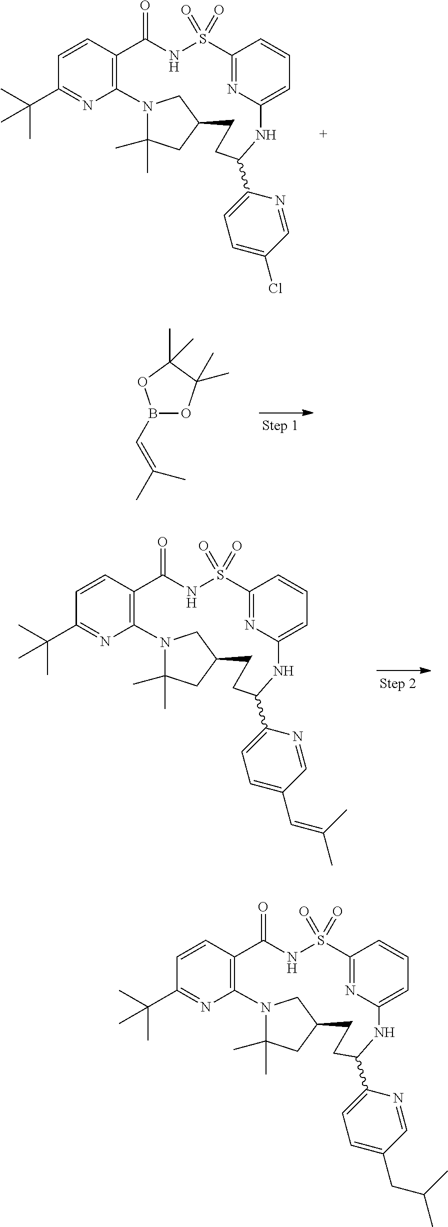

### Step 1: (14S)-8-tert-Butyl-12,12-dimethyl-17-[5-(2-methylprop-1-en-1-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione, Compound 183

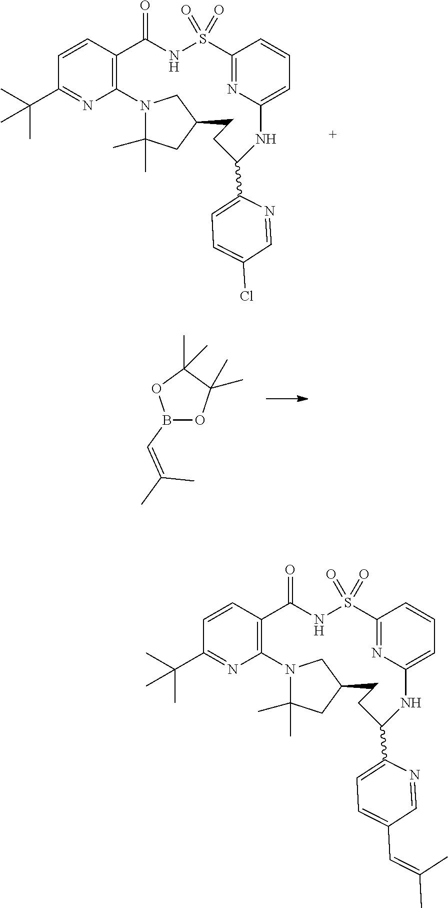 [SMILES: CC(C)(C)c1ccc2c(n1)N1C[C@@H](CCC(c3ccc(Cl)cn3)Nc3cccc(n3)S(=O)(=O)NC2=O)CC1(C)C ; CC(C)=CB1OC(C)(C)C(C)(C)O1 ; CC(C)=Cc1ccc(C2CC[C@@H]3CN(c4nc(C(C)(C)C)ccc4C(=O)NS(=O)(=O)c4cccc(n4)N2)C(C)(C)C3)nc1]

In a microwave vial (14S)-8-tert-butyl-17-(5-chloropyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (diastereomer 1, Compound 209, 72 mg, 0.1235 mmol) and 4,4,5,5-tetramethyl-2-(2-methylprop-1-enyl)-1,3,2-dioxaborolane (125 mg, 0.6866 mmol) were combined in DMSO (1.5 mL). Added to the mixture were [1,1′-bis(diphenylphosphino)ferrocene]dichloropalladium(II) (10 mg, 0.01367 mmol) and aqueous potassium carbonate (300 μL of 2 M, 0.600 mmol) and nitrogen was bubbled through the suspension for 1 minute. The reaction was capped and heated to 105° C. for 20 h. The mixture was filtered, and purified by reverse-phase preparative chromatography utilizing a C₁₈ column and a 20-80% gradient over 30 min of acetonitrile in water (+5 mM HCl) to afford (14S)-8-tert-butyl-12,12-dimethyl-17-[5-(2-methylprop-1-en-1-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione (33 mg, 44%). ESI-MS m/z calc. 602.3039, found 603.2 (M+1)⁺; Retention time: 1.76 minutes (LC method A).

### Step 2: (14S)-8-tert-Butyl-12,12-dimethyl-17-[5-(2-methylpropyl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione, Compound 181

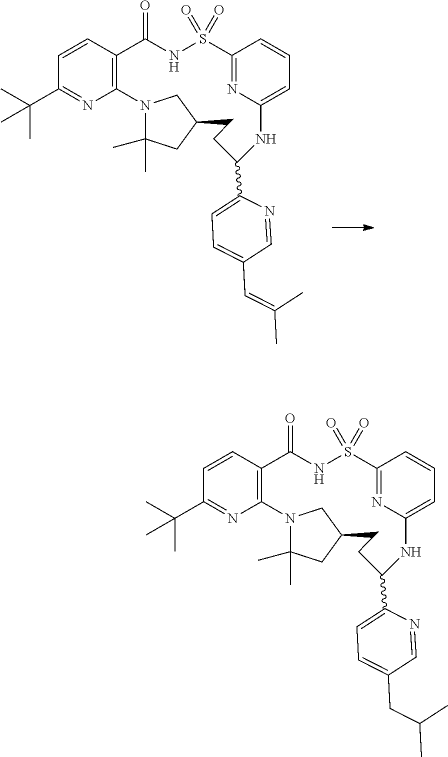

To a nitrogen purged 20 mL vial, (14S)-8-tert-butyl-12,12-dimethyl-17-[5-(2-methylprop-1-en-1-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione (Compound 183, 35 mg, 0.05748 mmol) was dissolved in MeOH (1.5 mL). To the nitrogen purged reaction solution was added palladium on carbon (5 mg, 10% w/w, 0.0047 mmol) and a balloon containing hydrogen gas was attached. Hydrogen gas was purged through the solution for 1 min prior to stirring under hydrogen balloons for 4 h at rt. The crude material was filtered through a Whatman filter disc and concentrated under a stream of nitrogen to give a viscous residue. This mixture was diluted with 1.5 mL DMSO and was then purified by reverse-phase preparative chromatography utilizing a C₁₈ column and a gradient of 20-80% over 30 min of acetonitrile in water (+5 mM HCl) to afford a white solid (14S)-8-tert-butyl-12,12-dimethyl-17-[5-(2-methylpropyl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione (18.3 mg, 52%). ¹H NMR (400 MHz, DMSO-d₆) δ 12.52 (s, 1H), 8.63 (d, J=2.1 Hz, 1H), 8.32 (d, J=8.3 Hz, 1H), 8.11 (d, J=9.3 Hz, 2H), 7.73 (dd, J 8.5, 7.3 Hz, 1H), 7.43 (d, J=7.9 Hz, 1H), 7.35 (d, J=7.3 Hz, 1H), 6.97 (d, J=8.5 Hz, 1H), 6.62 (d, J=7.9 Hz, 1H), 5.09 (s, 1H), 3.93 (s, 2H), 2.68 (s, 1H), 2.64 (d, J=7.2 Hz, 2H), 2.29 (q, J=10.2, 9.4 Hz, 1H), 2.22-2.08 (m, 1H), 1.88 (ddd, J=21.7, 12.6, 6.2 Hz, 2H), 1.82-1.70 (m, 1H), 1.54 (d, J=2.5 Hz, 6H), 1.50-1.38 (m, 1H), 1.26 (s, 9H), 1.21-1.08 (m, 1H), 0.86 (dd, J=6.6, 2.0 Hz, 6H). ESI-MS m/z calc. 604.3196, found 605.4 (M+1)⁺; Retention time: 1.78 minutes (LC method A).

### Example 27: Preparation of analogs related to (14S)-8-tert-butyl-17-(5-chloropyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 1, Compound 209

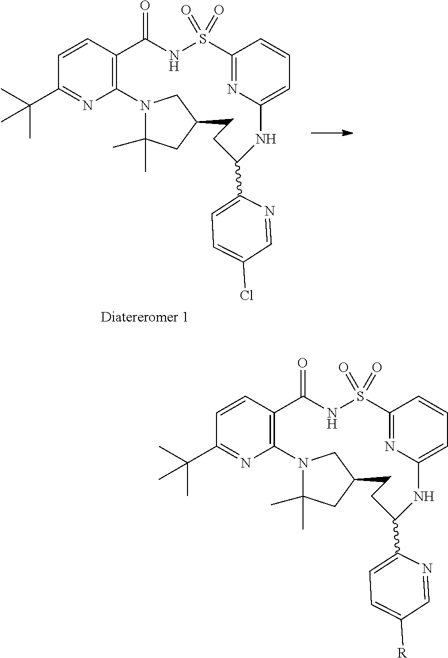

The following is a list of amine reagents that are commercially available:
Piperidine
Morpholine

The following is a list of boron reagents that are commercially available:
1-Methyl-3-(4,4,5,5-tetramethyl-1,3,2-dioxaborolan-2-yl)pyrazole
(2-Methylpyrazol-3-yl)boronic acid

The compounds in the following tables were prepared in a manner analogous to that described above using amine and boron reagents given in the tables above, and using (14S)-8-tert-butyl-17-(5-chloropyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 1, Compound 209 as a starting material.

| | | LCMS | | | |
|---|---|---|---|---|---|
| | | Retention | | | |
| Compound | | Time | Exact | | LCMS |
| Number | Structure | (min) | Mass | M + 1 | Method |
| Compound 199 | 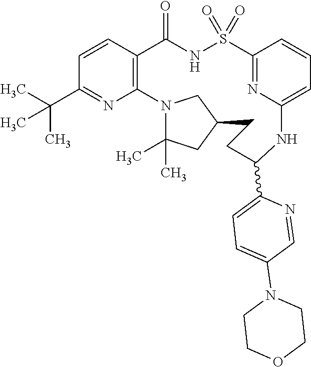 [SMILES: CC(C)(C)c1ccc2c(n1)N1C[C@@H](CCC(c3ccc(N4CCOCC4)cn3)Nc3cccc(n3)S(=O)(=O)NC2=O)CC1(C)C] | 1.56 | 633.31 | 634.2 | LC method A |
| Compound 185 | 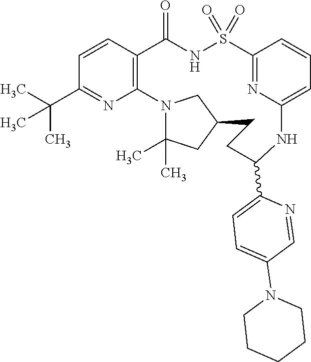 [SMILES: CC(C)(C)c1ccc2c(n1)N1C[C@@H](CCC(c3ccc(N4CCCCC4)cn3)Nc3cccc(n3)S(=O)(=O)NC2=O)CC1(C)C] | 1.74 | 631.33 | 632.5 | LC method A |
| Compound 60 | 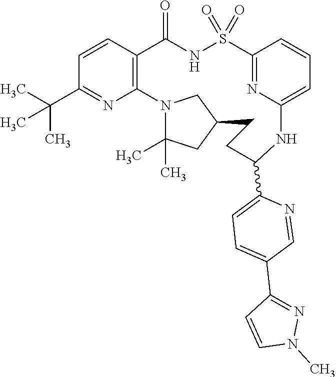 [SMILES: Cn1ccc(-c2ccc(C3CC[C@@H]4CN(c5nc(C(C)(C)C)ccc5C(=O)NS(=O)(=O)c5cccc(n5)N3)C(C)(C)C4)nc2)n1] | 1.74 | 628.294 | 629.2 | LC method A |
| Compound 58 | 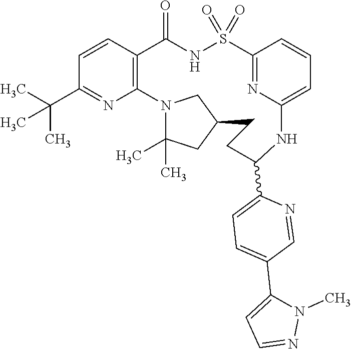 [SMILES: Cn1nccc1-c1ccc(C2CC[C@@H]3CN(c4nc(C(C)(C)C)ccc4C(=O)NS(=O)(=O)c4cccc(n4)N2)C(C)(C)C3)nc1] | 1.91 | 628.294 | 629.2 | LC method A |

### Example 28: Preparation of (14S)-17-[5-(aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 197

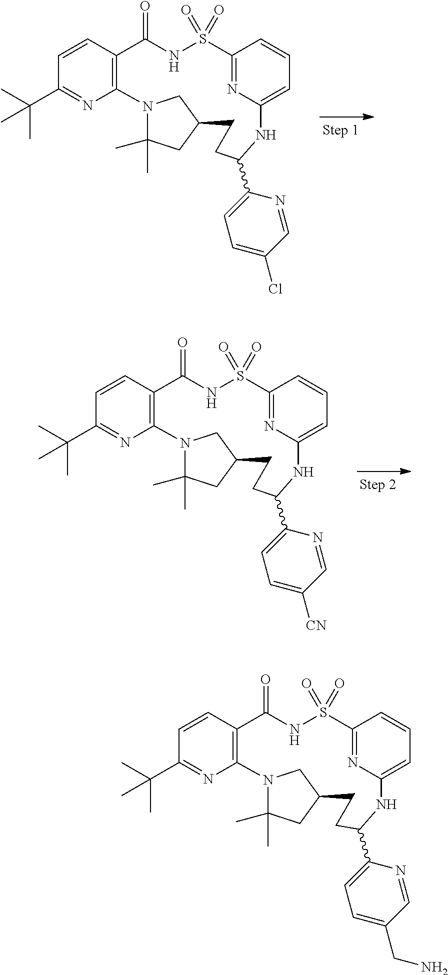 [SMILES: CC(C)(C)c1ccc2c(n1)N1C[C@@H](CCC(c3ccc(Cl)cn3)Nc3cccc(n3)S(=O)(=O)NC2=O)CC1(C)C ; CC(C)(C)c1ccc2c(n1)N1C[C@@H](CCC(c3ccc(C#N)cn3)Nc3cccc(n3)S(=O)(=O)NC2=O)CC1(C)C ; CC(C)(C)c1ccc2c(n1)N1C[C@@H](CCC(c3ccc(CN)cn3)Nc3cccc(n3)S(=O)(=O)NC2=O)CC1(C)C]

### Step 1: 6-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-3-carbonitrile, Compound 205

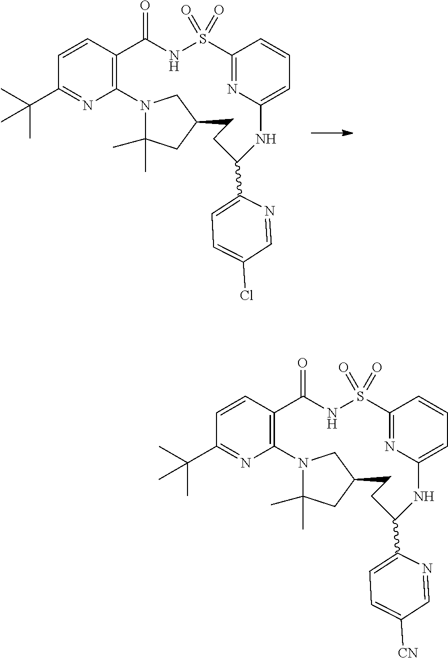 [SMILES: CC(C)(C)c1ccc2c(n1)N1C[C@@H](CCC(c3ccc(Cl)cn3)Nc3cccc(n3)S(=O)(=O)NC2=O)CC1(C)C ; CC(C)(C)c1ccc2c(n1)N1C[C@@H](CCC(c3ccc(C#N)cn3)Nc3cccc(n3)S(=O)(=O)NC2=O)CC1(C)C]

In a microwave vial (14S)-8-tert-butyl-17-(5-chloropyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (diastereomer 1, Compound 209, 25 mg, 0.0429 mmol) was dissolved in DMF (500 μL):water (5 μL) (99:1 v:v) and to the mixture was added dicyanozinc (4 μL, 0.0630 mmol) followed by dicyclohexyl-[2-(2,6-dimethoxyphenyl)phenyl]phosphane (2 mg, 0.00487 mmol) and Pd₂dba₃ (2 mg, 0.00218 mmol). The mixture was purged with nitrogen, capped and irradiated in the microwave for 30 minutes at 150° C. under microwave heating. Cooled to room temperature and the mixture was filtered, and purified by reverse-phase preparative chromatography utilizing a C₁₈ column and a gradient of 30-99% over 15 min of acetonitrile in water (+5 mM HCl) to afford 6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-3-carbonitrile (12.9 mg, 52%). ESI-MS m/z calc. 573.2522, found 574.5 (M+1)⁺; Retention time: 2.05 minutes (LC method A).

### Step 2: (14S)-17-[5-(Aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 197

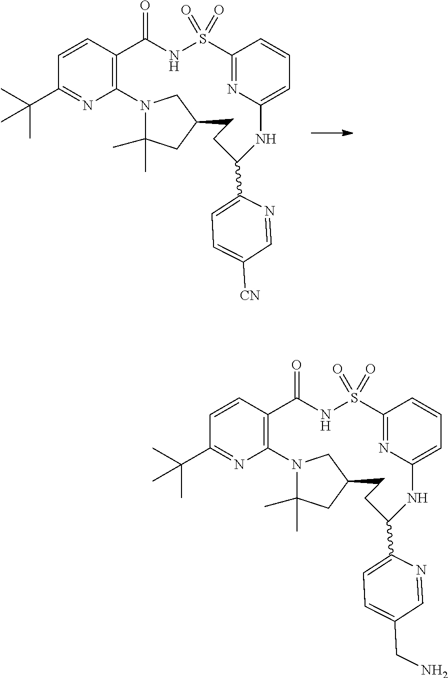 [SMILES: CC(C)(C)c1ccc2c(n1)N1C[C@@H](CCC(c3ccc(C#N)cn3)Nc3cccc(n3)S(=O)(=O)NC2=O)CC1(C)C ; CC(C)(C)c1ccc2c(n1)N1C[C@@H](CCC(c3ccc(CN)cn3)Nc3cccc(n3)S(=O)(=O)NC2=O)CC1(C)C]

In a microwave vial 6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-3-carbonitrile (Compound 205 (diastereomer 1), 9.2 mg, 0.01604 mmol) was dissolved in EtOH (750 μL) and purged with nitrogen for 5 minutes. To the mixture was added raney nickel (3 mg of 50% w/w, 0.0256 mmol) followed by a hydrogen balloon. The mixture was sealed and heated to 60° C. for 4 h. Cooled to room temperature and the mixture was filtered, and purified by reverse-phase preparative chromatography utilizing a C₁₈ column and a 1-70% gradient over 15 min of acetonitrile in water (+5 mM HCl) to afford (14S)-17-[5-(aminomethyl)pyridin-2-yl]-8-tert-butyl-12,12-dimethyl-22P-thia-3,9,11,18,23-pentaazatetracyclo[17.3.1.111,14.05,10]tetracosa-1(23),5(10),6,8,19,21-hexaene-2,2,4-trione (hydrochloride salt) (3.7 mg, 37%). ESI-MS m/z calc. 577.2835, found 578.2 (M+1)⁺; Retention time: 1.34 minutes (LC method A).

### Example 29: Preparation of analogs related to (14S)-8-tert-butyl-17-(5-chloropyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 2, Compound 208

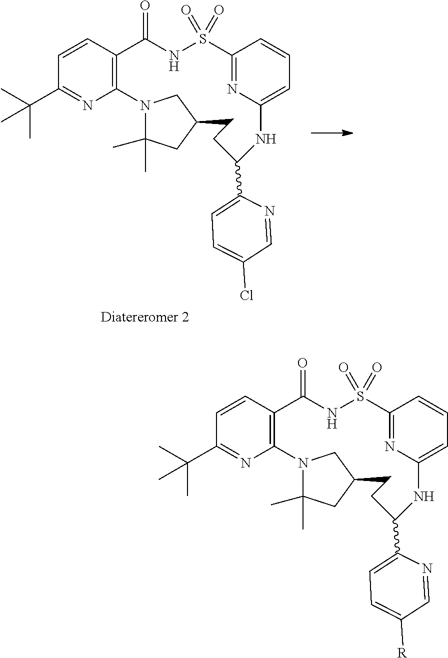

The compounds in the following tables were prepared in a manner analogous to that described above using (14S)-8-tert-butyl-17-(5-chloropyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (diastereomer 2, Compound 208) as a starting material.

| | | LCMS | | | |
|---|---|---|---|---|---|
| | | Retention | | | |
| Compound | | Time | Exact | | LCMS |
| Number | Structure | (min) | Mass | M + 1 | Method |
| Compound 57 | 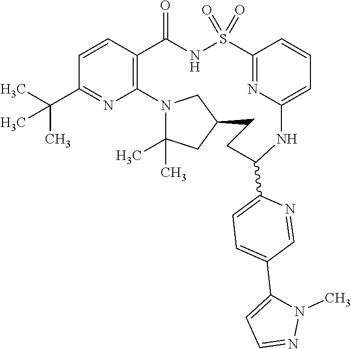 [SMILES: Cn1nccc1-c1ccc(C2CC[C@@H]3CN(c4nc(C(C)(C)C)ccc4C(=O)NS(=O)(=O)c4cccc(n4)N2)C(C)(C)C3)nc1] | 1.82 | 628.294 | 629.2 | LC method A |
| Compound 59 | 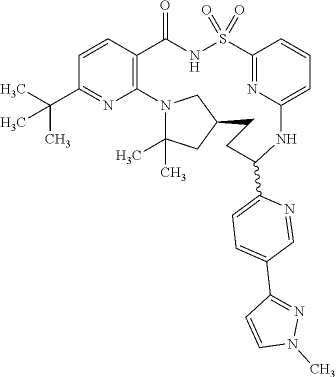 [SMILES: Cn1ccc(-c2ccc(C3CC[C@@H]4CN(c5nc(C(C)(C)C)ccc5C(=O)NS(=O)(=O)c5cccc(n5)N3)C(C)(C)C4)nc2)n1] | 1.64 | 628.294 | 629.2 | LC method A |
| Compound 180 | 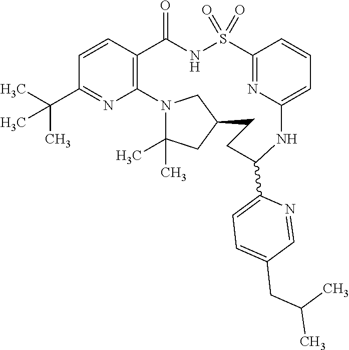 [SMILES: CC(C)Cc1ccc(C2CC[C@@H]3CN(c4nc(C(C)(C)C)ccc4C(=O)NS(=O)(=O)c4cccc(n4)N2)C(C)(C)C3)nc1] | 1.82 | 604.32 | 605.4 | LC method A |
| Compound 175 | 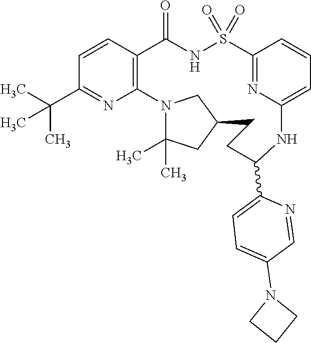 [SMILES: CC(C)(C)c1ccc2c(n1)N1C[C@@H](CCC(c3ccc(N4CCC4)cn3)Nc3cccc(n3)S(=O)(=O)NC2=O)CC1(C)C] | 1.72 | 603.299 | 604.4 | LC method A |
| Compound 182 | 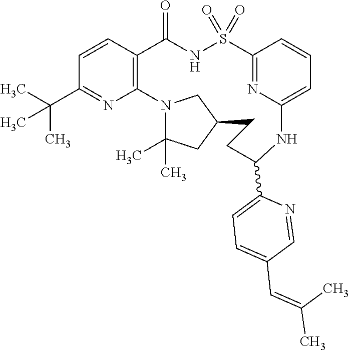 [SMILES: CC(C)=Cc1ccc(C2CC[C@@H]3CN(c4nc(C(C)(C)C)ccc4C(=O)NS(=O)(=O)c4cccc(n4)N2)C(C)(C)C3)nc1] | 1.82 | 602.304 | 603.2 | LC method A |
| Compound 184 | 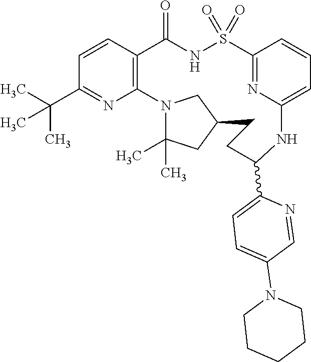 [SMILES: CC(C)(C)c1ccc2c(n1)N1C[C@@H](CCC(c3ccc(N4CCCCC4)cn3)Nc3cccc(n3)S(=O)(=O)NC2=O)CC1(C)C] | 1.73 | 631.33 | 632.5 | LC method A |
| Compound 196, hydrochloride salt | 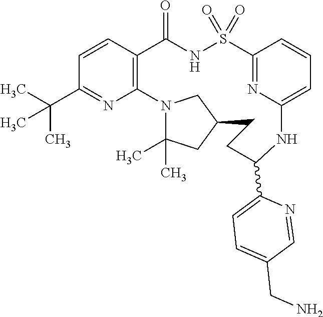 [SMILES: CC(C)(C)c1ccc2c(n1)N1C[C@@H](CCC(c3ccc(CN)cn3)Nc3cccc(n3)S(=O)(=O)NC2=O)CC1(C)C] | 1.51 | 577.284 | 578.2 | LC method A |
| Compound 198 | 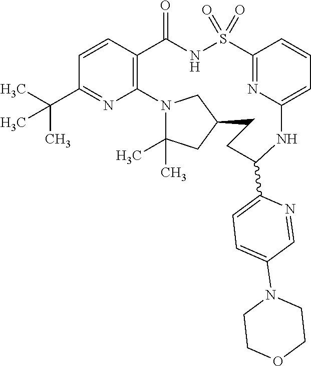 [SMILES: CC(C)(C)c1ccc2c(n1)N1C[C@@H](CCC(c3ccc(N4CCOCC4)cn3)Nc3cccc(n3)S(=O)(=O)NC2=O)CC1(C)C] | 1.62 | 633.31 | 634.2 | LC method A |
| Compound 200, hydrochloride salt | 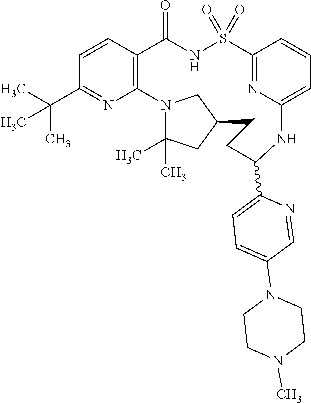 [SMILES: CN1CCN(c2ccc(C3CC[C@@H]4CN(c5nc(C(C)(C)C)ccc5C(=O)NS(=O)(=O)c5cccc(n5)N3)C(C)(C)C4)nc2)CC1] | 1.41 | 646.341 | 647.3 | LC method A |
| Compound 204 | 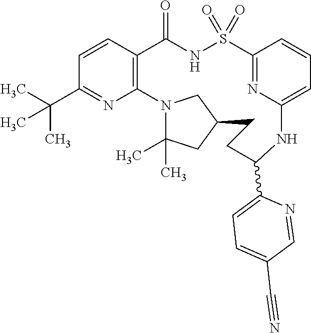 [SMILES: CC(C)(C)c1ccc2c(n1)N1C[C@@H](CCC(c3ccc(C#N)cn3)Nc3cccc(n3)S(=O)(=O)NC2=O)CC1(C)C] | 2.05 | 573.252 | 574.3 | LC method A |

| Compound | | |
|---|---|---|
| Number | Structure | NMR |
| Compound 57 | 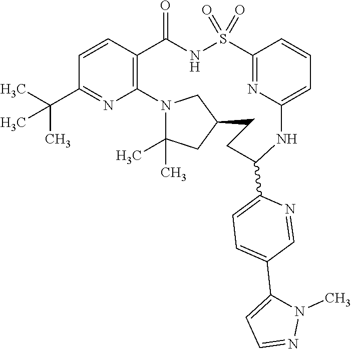 [SMILES: Cn1nccc1-c1ccc(C2CC[C@@H]3CN(c4nc(C(C)(C)C)ccc4C(=O)NS(=O)(=O)c4cccc(n4)N2)C(C)(C)C3)nc1] | ¹H NMR (400 MHz, DMSO-d₆) δ 12.31 (s, 1H), 8.76 (s, 1H), 8.05 (d, J = 7.5 Hz, 1H), 7.85 (s, 1H), 7.75 (s, 1H), 7.65 (t, J = 7.9 Hz, 1H), 7.50 (dd, J = 11.9, 1.8 Hz, 2H), 7.23 (d, J = 7.1 Hz, 1H), 6.90 (d, J = 8.5 Hz, 1H), 6.64 (d, J = 7.9 Hz, 1H), 6.55 (d, J = 1.9 Hz, 1H), 5.03 (d, J = 7.9 Hz, 1H), 3.88 (s, 3H), 3.12 (s, 2H), 2.33 (s, 1H), 2.32-2.08 (m, 2H), 1.87 (dd, J = 12.0, 5.8 Hz, 1H), 1.76 (dt, J = 11.4, 4.8 Hz, 1H), 1.61 (s, 3H), 1.58 (s, 1H), 1.54 (s, 3H), 1.30-1.24 (m, 9H), 1.21 (s, 1H). |
| Compound 59 | 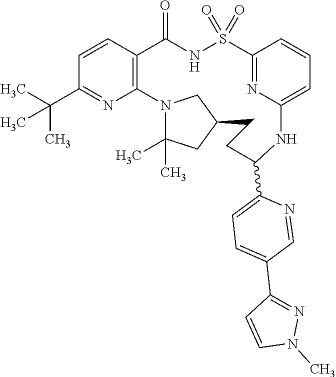 [SMILES: Cn1ccc(-c2ccc(C3CC[C@@H]4CN(c5nc(C(C)(C)C)ccc5C(=O)NS(=O)(=O)c5cccc(n5)N3)C(C)(C)C4)nc2)n1] | ¹H NMR (400 MHz, DMSO-d₆) δ 12.28 (s, 1H), 8.96 (s, 1H), 8.08 (d, J = 8.1 Hz, 1H), 7.77 (d, J = 2.3 Hz, 1H), 7.72 (d, J = 7.4 Hz, 1H), 7.62 (t, J = 8.0 Hz, 1H), 7.56 (d, J = 8.2 Hz, 1H), 7.49 (s, 1H), 7.20 (d, J = 7.1 Hz, 1H), 6.87 (d, J = 8.5 Hz, 1H), 6.78 (d, J = 2.2 Hz, 1H), 6.64 (d, J = 8.0 Hz, 1H), 4.92 (d, J = 57.9 Hz, 1H), 3.89 (s, 3H), 3.12 (s, 2H), 2.31 (d, J = 7.5 Hz, 1H), 2.17 (q, J = 14.2, 10.5 Hz, 2H), 1.86 (dd, J = 12.1, 5.8 Hz, 1H), 1.72 (dd, J = 13.5, 5.2 Hz, 1H), 1.61 (s, 3H), 1.57 (s, 2H), 1.53 (s, 3H), 1.28 (s, 9H). |
| Compound 180 | 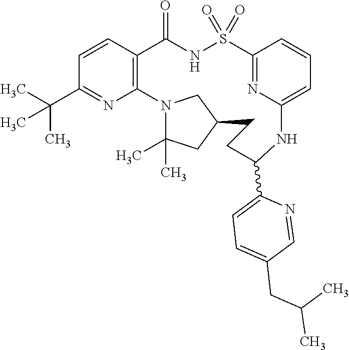 [SMILES: CC(C)Cc1ccc(C2CC[C@@H]3CN(c4nc(C(C)(C)C)ccc4C(=O)NS(=O)(=O)c4cccc(n4)N2)C(C)(C)C3)nc1] | ¹H NMR (400 MHz, DMSO-d₆) δ 12.49 (s, 1H), 8.52 (s, 1H), 8.13-7.75 (m, 2H), 7.72- 7.51 (m, 3H), 7.16 (d, J = 7.2 Hz, 1H), 6.93 (d, J = 8.5 Hz, 1H), 6.65 (d, J = 8.0 Hz, 1H), 5.37 (s, 1H), 3.29-3.20 (m, 2H), 2.74-2.67 (m, 1H), 2.54 (s, 1H), 2.34-2.26 (m, 1H), 2.00- 1.91 (m, 2H), 1.91-1.82 (m, 2H), 1.75 (d, J = 14.0 Hz, 1H), 1.66 (s, 3H), 1.57 (t, J = 12.3 Hz, 1H), 1.51 (s, 3H), 1.46 (d, J = 12.2 Hz, 1H), 1.28 (s, 9H), 0.85 (d, J = 6.6 Hz, 6H). |

### Example 30: Preparation of (14S)-17-(5-bromopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 172 (diastereomer 1) and Compound 171 (diastereomer 2)

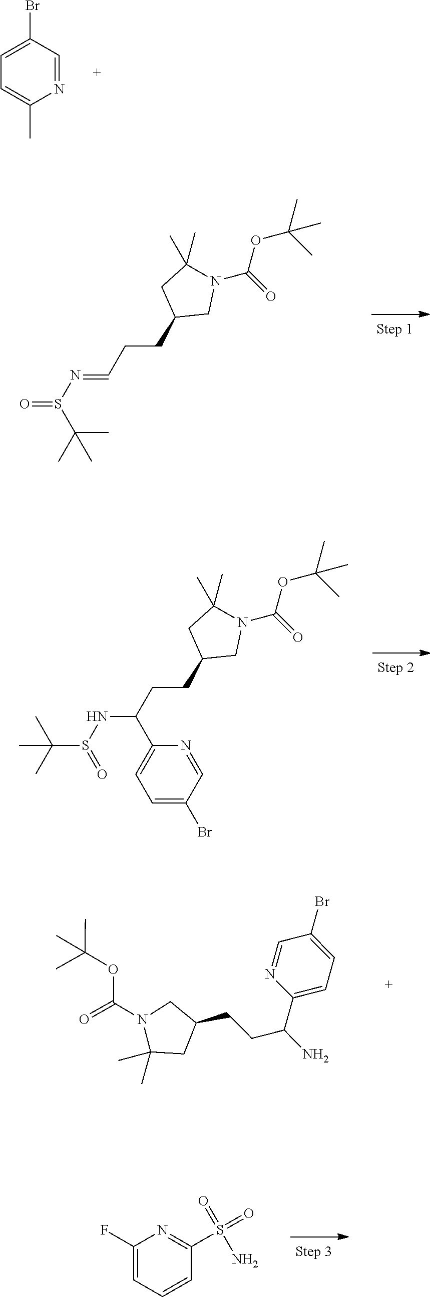

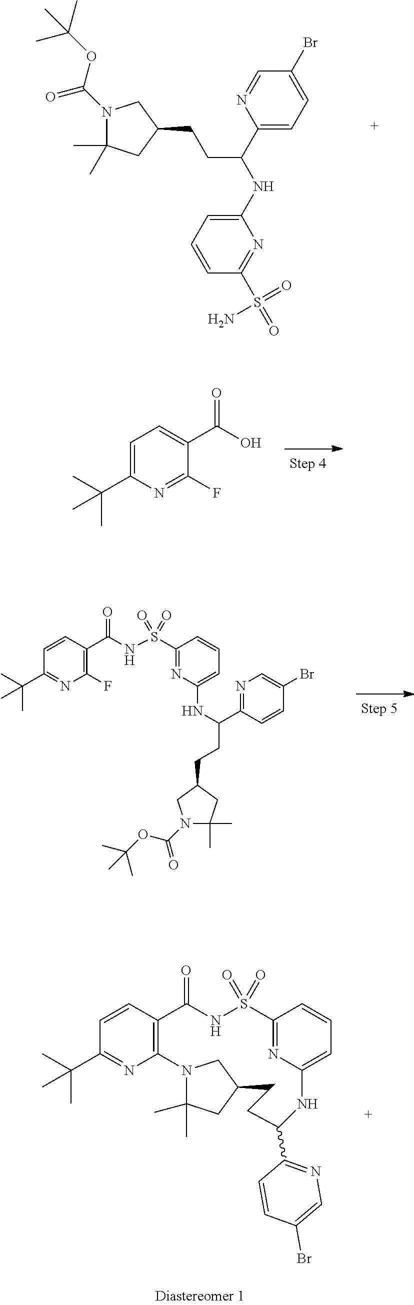 [SMILES: CC(C)(C)OC(=O)N1C[C@@H](CCC(Nc2cccc(S(N)(=O)=O)n2)c2ccc(Br)cn2)CC1(C)C ; CC(C)(C)c1ccc(C(=O)O)c(F)n1 ; CC(C)(C)OC(=O)N1C[C@@H](CCC(Nc2cccc(S(=O)(=O)NC(=O)c3ccc(C(C)(C)C)nc3F)n2)c2ccc(Br)cn2)CC1(C)C ; CC(C)(C)c1ccc2c(n1)N1C[C@@H](CCC(c3ccc(Br)cn3)Nc3cccc(n3)S(=O)(=O)NC2=O)CC1(C)C]

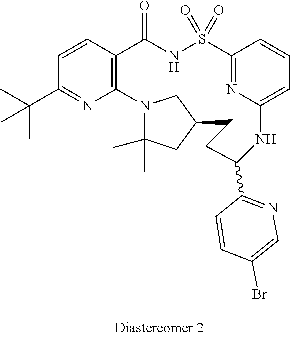 [SMILES: CC(C)(C)c1ccc2c(n1)N1C[C@@H](CCC(c3ccc(Br)cn3)Nc3cccc(n3)S(=O)(=O)NC2=O)CC1(C)C]

### Step 1: tert-Butyl (4S)-4-[3-(5-bromo-2-pyridyl)-3-(tert-butylsulfinylamino)propyl]-2,2-dimethyl-pyrrolidine-1-carboxylate

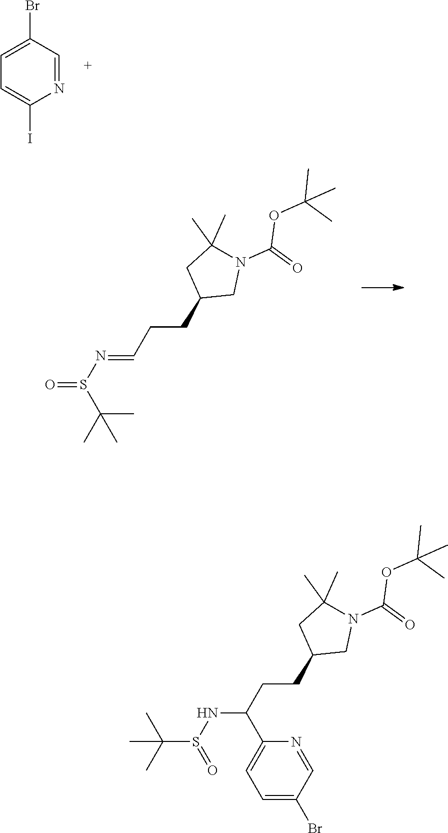

5-bromo-2-iodo-pyridine (11.6 g, 39.63 mmol) was dissolved in diethyl ether (360 mL) and the solution was cooled in a dry ice acetone bath. Precipitate emerged and stirring was adjusted to make the suspension well dispersed. n-BuLi (16 mL of 2.5 M in hexanes, 40.00 mmol) was added in dropwise quickly fashion. The dark brownish suspension was stirred below −70° C. for 45 min. tert-Butyl (4S)-4-[(3E)-3-tert-butylsulfinyliminopropyl]-2,2-dimethyl-pyrrolidine-1-carboxylate (7.2 g, 20.081 mmol) in THF (15 mL+3 mL rinse) was added. NH₄Cl (saturated aqueous 20 mL) was added to quench the reaction, followed by water (150 mL) and EtOAc (200 mL). After warming up to rt, layers were separated. The combined organics were dried over anhydrous MgSO₄, filtered and concentrated. The residue was purified by silica gel chromatography (120 g column), using 5-80% EtOAc in hexanes to afford tert-butyl (4S)-4-[3-(5-bromo-2-pyridyl)-3-(tert-butylsulfinylamino)propyl]-2,2-dimethyl-pyrrolidine-1-carboxylate as a light brownish foam (9.8 g, 90%). ESI-MS m/z calc. 517.1797, found 518.6 (M+1)⁺; Retention time: 3.93 minutes (LC method B).

### Step 2: tert-Butyl (4S)-4-[3-amino-3-(5-bromo-2-pyridyl)propyl]-2,2-dimethyl-pyrrolidine-1-carboxylate

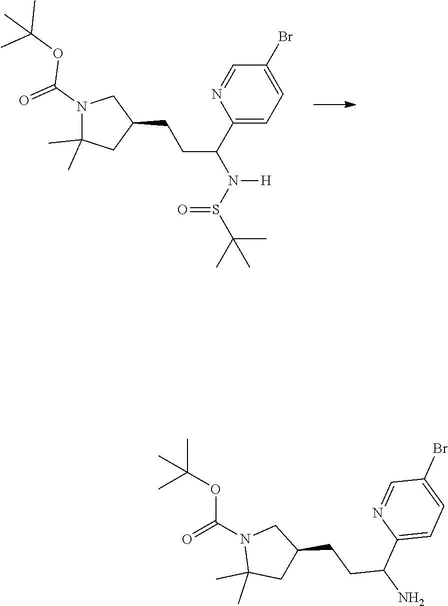 [SMILES: CC(C)(C)OC(=O)N1C[C@@H](CCC(NS(=O)C(C)(C)C)c2ccc(Br)cn2)CC1(C)C ; CC(C)(C)OC(=O)N1C[C@@H](CCC(N)c2ccc(Br)cn2)CC1(C)C]

To a stirring solution of tert-butyl (4S)-4-[3-(5-bromo-2-pyridyl)-3-(tert-butylsulfinylamino)propyl]-2,2-dimethyl-pyrrolidine-1-carboxylate (12.526 g, 24.250 mmol) in a mixture of THF (125 mL) and water (32 mL) at room temperature under ambient conditions was added iodine (2.172 g, 8.5576 mmol). The reaction mixture was heated to 55° C. for 2 h. After cooling to room temperature, the reaction mixture was poured into a mixture of saturated aqueous sodium bicarbonate (230 mL) and saturated aqueous Na₂S₂O₃ (60 mL). Volatiles were removed under vacuum, and the residual aqueous layer was extracted with ethyl acetate (3×200 mL). Combined organic layers were washed with brine (120 mL), dried over anhydrous sodium sulfate and concentrated to afford tert-butyl (4S)-4-[3-amino-3-(5-bromo-2-pyridyl)propyl]-2,2-dimethyl-pyrrolidine-1-carboxylate (11.353 g, 100%) as amber oil. The product was carried to the next step without further purification. ESI-MS m/z calc. 411.1521, found 412.3 (M+1)⁺; Retention time: 4.5 minutes (LC method C).

### Step 3: tert-Butyl (4S)-4-[3-(5-bromo-2-pyridyl)-3-[(6-sulfamoyl-2-pyridyl)amino]propyl]-2,2-dimethyl-pyrrolidine-1-carboxylate

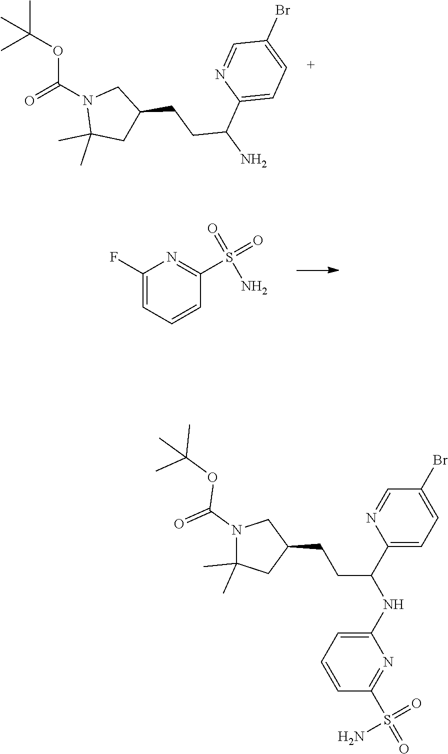 [SMILES: CC(C)(C)OC(=O)N1C[C@@H](CCC(N)c2ccc(Br)cn2)CC1(C)C ; NS(=O)(=O)c1cccc(F)n1 ; CC(C)(C)OC(=O)N1C[C@@H](CCC(Nc2cccc(S(N)(=O)=O)n2)c2ccc(Br)cn2)CC1(C)C]

To a stirring solution of tert-butyl (4S)-4-[3-amino-3-(5-bromo-2-pyridyl)propyl]-2,2-dimethyl-pyrrolidine-1-carboxylate (11.353 g, 24.778 mmol) and 6-fluoropyridine-2-sulfonamide (6.565 g, 37.265 mmol) in anhydrous DMSO (40 mL) at room temperature under nitrogen was added DIEA (14 mL, 80.376 mmol). The reaction mixture was heated to 125° C. for 24 h. After cooling to room temperature, the reaction mixture was poured into a mixture of water (200 mL) and brine (300 mL). The product was extracted with ethyl acetate (3×250 mL). Combined organic layers were washed with brine (150 mL), dried over anhydrous sodium sulfate and concentrated. The crude was purified by silica gel chromatography using 0-25% acetone gradient in hexanes, followed by the reverse phase HPLC using 50-100% acetonitrile gradient in water (0.15% TFA buffer; C₁₈ Varian column; 60 mL/min.). All fractions containing the purified product were combined and basified with saturated aqueous sodium bicarbonate to pH˜8. Volatiles were removed under vacuum, and the residual aqueous layer was extracted with ethyl acetate (3×250 mL). Combined organic layers were washed with brine (150 mL), dried over anhydrous sodium sulfate and concentrated to afford tert-butyl (4S)-4-[3-(5-bromo-2-pyridyl)-3-[(6-sulfamoyl-2-pyridyl)amino]propyl]-2,2-dimethyl-pyrrolidine-1-carboxylate (9.124 g, 61%) as pale yellow solid. ¹H NMR (500 MHz, DMSO-d₆) δ 8.64 (d, J=2.4 Hz, 1H), 7.94 (dd, J=8.4, 2.4 Hz, 1H), 7.55-7.45 (m, 3H), 7.02 (s, 2H), 6.96 (d, J=7.2 Hz, 1H), 6.70 (d, J=8.4 Hz, 1H), 5.14 (s, 1H), 3.59-3.45 (m, 1H), 2.82-2.67 (m, 1H), 2.15-2.01 (m, 1H), 1.94-1.75 (m, 3H), 1.41-1.34 (m, 11H), 1.29-1.24 (m, 4H), 1.22 (s, 3H). ESI-MS m/z calc. 567.1515, found 568.3 (M+1)⁺; Retention time: 2.74 minutes (LC method B).

### Step 4: tert-Butyl (4S)-4-[3-(5-bromo-2-pyridyl)-3-[[6-[(6-tert-butyl-2-fluoro-pyridine-3-carbonyl)sulfamoyl]-2-pyridyl]amino]propyl]-2,2-dimethyl-pyrrolidine-1-carboxylate

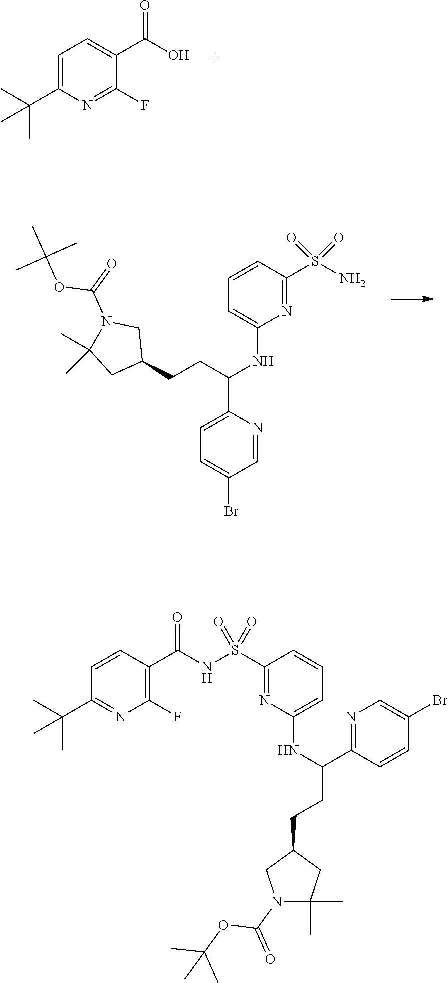 [SMILES: CC(C)(C)c1ccc(C(=O)O)c(F)n1 ; CC(C)(C)OC(=O)N1C[C@@H](CCC(Nc2cccc(S(N)(=O)=O)n2)c2ccc(Br)cn2)CC1(C)C ; CC(C)(C)OC(=O)N1C[C@@H](CCC(Nc2cccc(S(=O)(=O)NC(=O)c3ccc(C(C)(C)C)nc3F)n2)c2ccc(Br)cn2)CC1(C)C]

To a solution of 6-tert-butyl-2-fluoro-pyridine-3-carboxylic acid (1.95 g, 9.888 mmol) in THF (45 mL) was added CDI (1.56 g, 9.621 mmol) and the mixture was stirred at rt for 20 h. Then tert-butyl (4S)-4-[3-(5-bromo-2-pyridyl)-3-[(6-sulfamoyl-2-pyridyl)amino]propyl]-2,2-dimethyl-pyrrolidine-1-carboxylate (3.0 g, 5.277 mmol) was added followed by DBU (3.23 mL, 21.60 mmol) and the resulting mixture was stirred for 20 h at rt. The reaction was diluted with ethyl acetate and washed with a saturated aqueous sodium bicarbonate solution. The organic layer was further washed with a 10% citric acid solution followed by brine. The organics were separated, dried over sodium sulfate, and evaporated. The crude material was then purified on silica gel chromatography (220 gram column) using a gradient from 10% ethyl acetate in hexanes to 100% ethyl acetate to afford a residue which was placed under high vacuum pump for 3 h to afford tert-butyl (4S)-4-[3-(5-bromo-2-pyridyl)-3-[[6-[(6-tert-butyl-2-fluoro-pyridine-3-carbonyl)sulfamoyl]-2-pyridyl]amino]propyl]-2,2-dimethyl-pyrrolidine-1-carboxylate as an off-white solid (diastereomeric mixture, 3.5 g, 89%). ESI-MS m/z calc. 746.22614, found 747.6 (M+1)⁺; Retention time: 2.35 minutes (LC method A).

### Step 5: (14S)-17-(5-Bromopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 172 (diastereomer 1) and (14S)-17-(5-bromopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 171 (diastereomer 2)

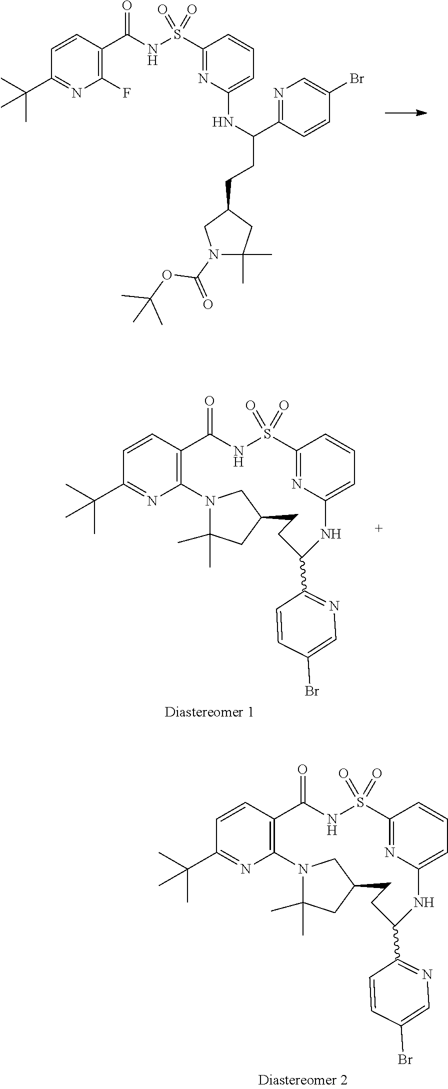 [SMILES: CC(C)(C)OC(=O)N1C[C@@H](CCC(Nc2cccc(S(=O)(=O)NC(=O)c3ccc(C(C)(C)C)nc3F)n2)c2ccc(Br)cn2)CC1(C)C ; CC(C)(C)c1ccc2c(n1)N1C[C@@H](CCC(c3ccc(Br)cn3)Nc3cccc(n3)S(=O)(=O)NC2=O)CC1(C)C ; CC(C)(C)c1ccc2c(n1)N1C[C@@H](CCC(c3ccc(Br)cn3)Nc3cccc(n3)S(=O)(=O)NC2=O)CC1(C)C]

Stage 1: tert-Butyl (4S)-4-[3-(5-bromo-2-pyridyl)-3-[[6-[(6-tert-butyl-2-fluoro-pyridine-3-carbonyl)sulfamoyl]-2-pyridyl]amino]propyl]-2,2-dimethyl-pyrrolidine-1-carboxylate (2.22 g, 2.969 mmol) was dissolved in DCM (60 mL) and to the mixture was added TFA (7 mL, 90.86 mmol) and stirred at room temperature. After 30 min, the mixture was evaporated to dryness, then diluted with diethyl ether (30 mL×2), and reconcentrated. The material was then placed on the high vacuum pump for 2 h to afford the intermediate N-[[6-[[1-(5-bromo-2-pyridyl)-3-[(3S)-5,5-dimethylpyrrolidin-3-yl]propyl]amino]-2-pyridyl]sulfonyl]-6-tert-butyl-2-fluoro-pyridine-3-carboxamide ESI-MS m/z calc. 646.1737, found 647.2 (M+1)⁺; Retention time: 1.51 minutes (LC method A) as an off-white solid. Stage 2: Combined material from Step 1 and K₂CO₃ (4.35 g, 31.47 mmol), 3 Å molecular sieves and NMP (50 mL) in a vial, purged with nitrogen, capped, heated to 150° C. and stirred for 20 h. Cooled to room temperature and the mixture was diluted with ethyl acetate and water. The organic layer was extracted (2×) and was further washed with 10% citric acid solution followed by brine. The organics were separated, dried over sodium sulfate, evaporated to a light brown oil. This residue was purified on silica gel chromatography (220 gram column) using a gradient from 10% ethyl acetate in hexanes to 100% ethyl acetate to afford two products, diastereomer separation into two single enantiomers.

Diastereomer 1: less polar, white solid, (14S)-17-(5-bromopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (601.6 mg, 63%). ¹H NMR (400 MHz, DMSO-d₆) δ 12.28 (s, 1H), 8.68 (d, J=2.4 Hz, 1H), 8.05-7.94 (m, 1H), 7.75 (d, J=7.9 Hz, 1H), 7.63 (t, J=7.9 Hz, 1H), 7.52 (d, J=8.5 Hz, 2H), 7.21 (d, J=7.3 Hz, 1H), 6.85 (d, J=8.5 Hz, 1H), 6.63 (d, J=7.9 Hz, 1H), 5.14-4.75 (m, 1H), 3.10 (s, 2H), 2.29 (d, J=8.9 Hz, 1H), 2.15 (s, 1H), 1.85 (dd, J=11.9, 5.9 Hz, 1H), 1.76-1.68 (m, 1H), 1.60 (s, 3H), 1.53 (s, 5H), 1.27 (s, 9H), 1.23 (t, J=3.8 Hz, 1H). ESI-MS m/z calc. 626.1675, found 627.3 (M+1)⁺; Retention time: 2.28 minutes (LC method A).

Diastereomer 2: more polar, white solid, (14S)-17-(5-bromopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (561.3 mg, 59%). ¹H NMR (400 MHz, DMSO-d₆) δ 12.49 (s, 1H), 8.73-8.58 (m, 1H), 8.00 (dd, J=8.4, 2.4 Hz, 1H), 7.75-7.59 (m, 3H), 7.42 (d, J=8.4 Hz, 1H), 7.11 (d, J=7.2 Hz, 1H), 6.87 (d, J=8.5 Hz, 1H), 6.65 (d, J=8.0 Hz, 1H), 5.24 (q, J=8.8 Hz, 1H), 3.19 (t, J=8.5 Hz, 1H), 2.75 (t, J=10.4 Hz, 1H), 2.27-2.15 (m, 1H), 1.90 (d, J=10.0 Hz, 2H), 1.84 (dd, J=11.7, 5.1 Hz, 1H), 1.73 (d, J=14.7 Hz, 1H), 1.64 (s, 3H), 1.57 (t, J=12.3 Hz, 1H), 1.51 (s, 3H), 1.28 (s, 9H), 1.23 (s, 1H). ESI-MS m/z calc. 626.1675, found 627.2 (M+1)⁺; Retention time: 2.22 minutes (LC method A).

### Example 31: Preparation of (14S)-8-tert-butyl-12,12-dimethyl-17-[5-(piperidin-4-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione, Compound 154

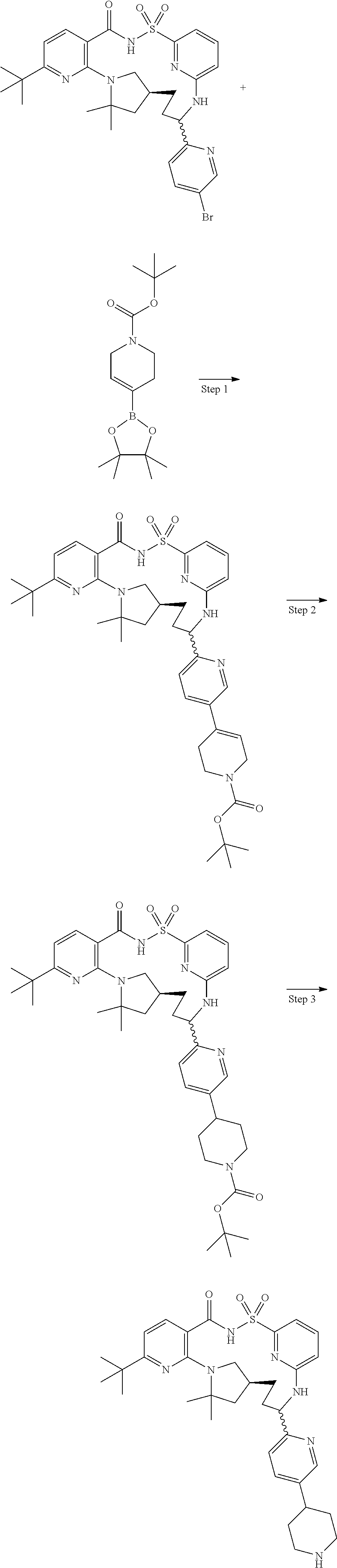 [SMILES: CC(C)(C)c1ccc2c(n1)N1C[C@@H](CCC(c3ccc(Br)cn3)Nc3cccc(n3)S(=O)(=O)NC2=O)CC1(C)C ; CC(C)(C)OC(=O)N1CC=C(B2OC(C)(C)C(C)(C)O2)CC1 ; CC(C)(C)OC(=O)N1CC=C(c2ccc(C3CC[C@@H]4CN(c5nc(C(C)(C)C)ccc5C(=O)NS(=O)(=O)c5cccc(n5)N3)C(C)(C)C4)nc2)CC1 ; CC(C)(C)OC(=O)N1CCC(c2ccc(C3CC[C@@H]4CN(c5nc(C(C)(C)C)ccc5C(=O)NS(=O)(=O)c5cccc(n5)N3)C(C)(C)C4)nc2)CC1 ; CC(C)(C)c1ccc2c(n1)N1C[C@@H](CCC(c3ccc(C4CCNCC4)cn3)Nc3cccc(n3)S(=O)(=O)NC2=O)CC1(C)C]

### Step 1: tert-Butyl 6-[(14S)-8-tert-butyl-12,12-dimethyl-2,2,4-trioxo-W-thia-3,9,11,18,23-pentaazatetracyclo[17.3.1.111,14.05,10]tetracosa-1(23),5(10),6,8,19,21-hexaen-17-yl]-1′,2′,3′,6′-tetrahydro-[3,4′-bipyridine]-1′-carboxylate

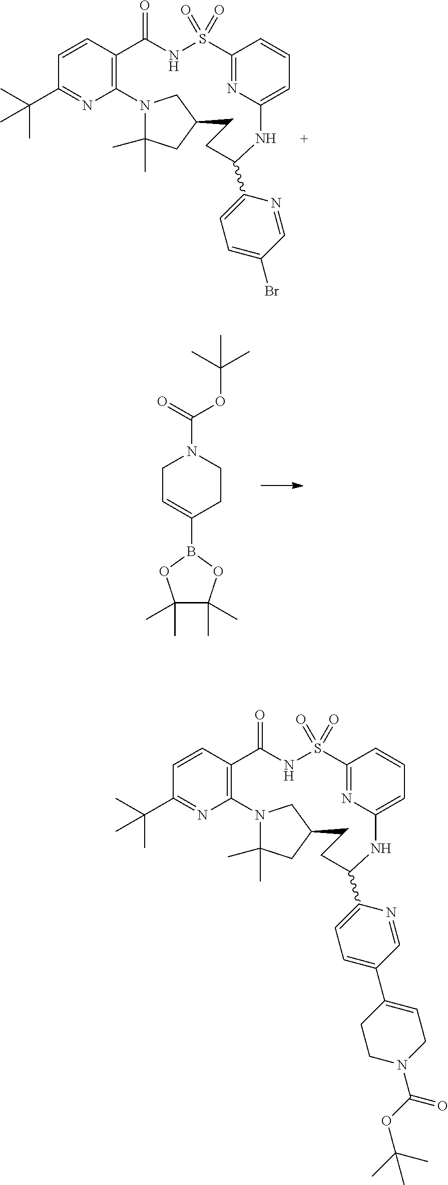 [SMILES: CC(C)(C)c1ccc2c(n1)N1C[C@@H](CCC(c3ccc(Br)cn3)Nc3cccc(n3)S(=O)(=O)NC2=O)CC1(C)C ; CC(C)(C)OC(=O)N1CC=C(B2OC(C)(C)C(C)(C)O2)CC1 ; CC(C)(C)OC(=O)N1CC=C(c2ccc(C3CC[C@@H]4CN(c5nc(C(C)(C)C)ccc5C(=O)NS(=O)(=O)c5cccc(n5)N3)C(C)(C)C4)nc2)CC1]

In a microwave vial (14S)-17-(5-bromopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 172 (diastereomer 1), 30 mg, 0.0468 mmol) and tert-butyl 4-(4,4,5,5-tetramethyl-1,3,2-dioxaborolan-2-yl)-3,6-dihydro-2H-pyridine-1-carboxylate (23 mg, 0.0744 mmol) were combined in DMSO (1000 μL). Added to the mixture were [1,1′-bis(diphenylphosphino)ferrocene]dichloropalladium(II) (4.5 mg, 0.0061 mmol) and aqueous potassium carbonate (200 μL of 2 M, 0.400 mmol) and nitrogen was bubbled through the suspension for 1 minute. The reaction was capped and heated to 110° C. for 20 h. The mixture was filtered, and purified by reverse-phase preparative chromatography utilizing a C₁₈ column and a 20-80% gradient over 30 min of acetonitrile in water (+5 mM HCl) to afford tert-butyl 6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-1′,2′,3′,6′-tetrahydro-[3,4′-bipyridine]-1′-carboxylate (21.0 mg, 61%). ESI-MS m/z calc. 729.36725, found 730.2 (M+1)⁺; Retention time: 1.97 minutes (LC method A).

### Step 2: tert-Butyl 4-{6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-3-yl}piperidine-1-carboxylate

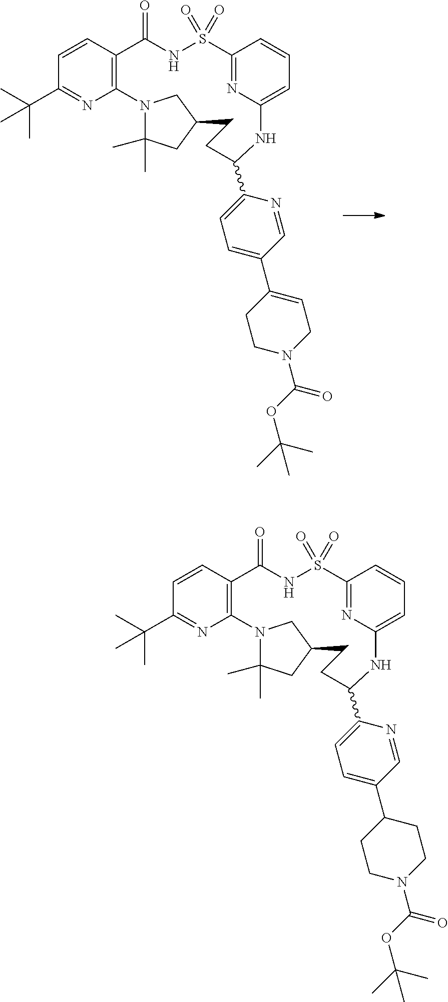 [SMILES: CC(C)(C)OC(=O)N1CC=C(c2ccc(C3CC[C@@H]4CN(c5nc(C(C)(C)C)ccc5C(=O)NS(=O)(=O)c5cccc(n5)N3)C(C)(C)C4)nc2)CC1 ; CC(C)(C)OC(=O)N1CCC(c2ccc(C3CC[C@@H]4CN(c5nc(C(C)(C)C)ccc5C(=O)NS(=O)(=O)c5cccc(n5)N3)C(C)(C)C4)nc2)CC1]

To a nitrogen purged 20 mL vial tert-butyl 6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-1′,2′,3′,6′-tetrahydro-[3,4′-bipyridine]-1′-carboxylate (21.0 mg, 0.02848 mmol) (originating from Compound 172 (diastereomer 1)) was dissolved in ethanol (1000 μL). To the nitrogen purged reaction solution was added palladium on carbon (10% w/w, 5 mg, 0.0047 mmol) and a balloon containing hydrogen gas was attached. Hydrogen gas was purged through the solution for 1 min prior to stirring under a hydrogen balloon for 3 h at rt. The crude material was filtered through a Whatman filter disc and concentrated under a stream of nitrogen to give a viscous residue tert-butyl 4-{6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-3-yl}piperidine-1-carboxylate (20.5 mg, 98%). ESI-MS m/z calc. 731.3829, found 732.2 (M+1)⁺; Retention time: 1.42 minutes (LC method A).

### Step 3: (14S)-8-tert-Butyl-12,12-dimethyl-17-[5-(piperidin-4-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione, Compound 154

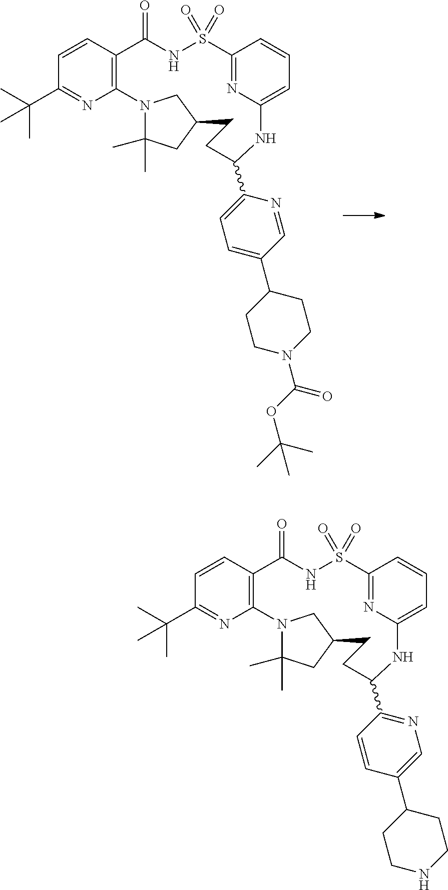 [SMILES: CC(C)(C)OC(=O)N1CCC(c2ccc(C3CC[C@@H]4CN(c5nc(C(C)(C)C)ccc5C(=O)NS(=O)(=O)c5cccc(n5)N3)C(C)(C)C4)nc2)CC1 ; CC(C)(C)c1ccc2c(n1)N1C[C@@H](CCC(c3ccc(C4CCNCC4)cn3)Nc3cccc(n3)S(=O)(=O)NC2=O)CC1(C)C]

tert-Butyl 4-{6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-3-yl}piperidine-1-carboxylate (originating from Compound 172 (diastereomer 1), 23 mg, 0.0314 mmol) from peak 1 was dissolved in DCM (1.0 mL) and to the mixture was added TFA (1.5 mL, 19.47 mmol) and stirred at room temperature. After 30 min, the mixture was evaporated to dryness, and then the residue was purified by reverse-phase preparative chromatography utilizing a C₁₈ column and a 1-70% gradient over 15 min of acetonitrile in water (+5 mM HCl) to afford the desired product as a white solid, (14S)-8-tert-butyl-12,12-dimethyl-17-[5-(piperidin-4-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (19.0 mg, 90%). ¹H NMR (400 MHz, DMSO-d₆) δ 12.43 (s, 1H), 9.19-8.72 (m, 2H), 8.62 (d, J=12.2 Hz, 1H), 8.40-7.78 (m, 3H), 7.71 (q, J=5.5, 3.3 Hz, 1H), 7.63 (d, J=8.0 Hz, 1H), 7.18 (d, J=7.2 Hz, 1H), 6.95 (dd, J=10.3, 5.4 Hz, 1H), 6.66 (d, J=8.0 Hz, 1H), 5.45 (s, 1H), 3.36 (d, J=12.7 Hz, 3H), 2.99 (d, J=14.2 Hz, 1H), 2.93 (d, J=12.1 Hz, 1H), 2.71 (s, 1H), 2.46-2.32 (m, 1H), 2.06-1.95 (m, 4H), 1.93-1.73 (m, 4H), 1.66 (s, 3H), 1.57 (t, J=12.4 Hz, 1H), 1.51 (s, 3H), 1.45 (d, J=11.4 Hz, 1H), 1.28 (s, 9H). ESI-MS m/z calc. 631.33044, found 632.2 (M+1)⁺; Retention time: 1.42 minutes (LC method A).

The following is a list of boron reagents that are commercially available:
tert-Butyl 3-(4,4,5,5-tetramethyl-1,3,2-dioxaborolan-2-yl)azetidine-1-carboxylate
2-(3,6-Dihydro-2H-pyran-4-yl)-4,4,5,5-tetramethyl-1,3,2-dioxaborolane
Cyclopropylboronic acid
3-(4,4,5,5-Tetramethyl-1,3,2-dioxaborolan-2-yl)propanenitrile
4-(4,4,5,5-Tetramethyl-1,3,2-dioxaborolan-2-yl)-1H-pyrazole
1H-Pyrazol-3-ylboronic acid
tert-Butyl N-[2-(4,4,5,5-tetramethyl-1,3,2-dioxaborolan-2-yl)ethyl]carbamate
2-(2-Methoxyethyl)-4,4,5,5-tetramethyl-1,3,2-dioxaborolane (Dimethylamino)methyl-trifluoro-boranuide (Potassium salt)
Methyl 3-(4,4,5,5-tetramethyl-1,3,2-dioxaborolan-2-yl)propanoate

The compounds in the following tables were prepared in a manner analogous to that described above using boron reagents given in the table above and using (14S)-17-(5-bromopyridin-2-yl)-8-tert-butyl-12,12-dimethyl-2λ⁶-thia-3,9,11,18,23-pentaazatetracyclo[17.3.1.111,14.05,10]tetracosa-1 (23),5(10),6,8,19,21-hexaene-2,2,4-trione, diastereomer 1, Compound 172 as a starting material:

| Compound | | | | | |
|---|---|---|---|---|---|
| Number | Structure | NMR | | | |
| Compound 168 | 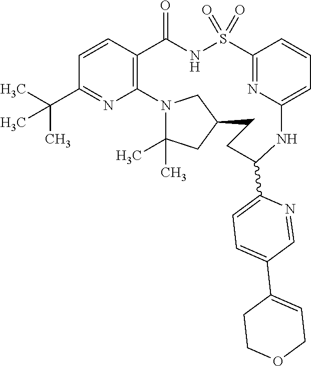 [SMILES: CC(C)(C)c1ccc2c(n1)N1C[C@@H](CCC(c3ccc(C4=CCOCC4)cn3)Nc3cccc(n3)S(=O)(=O)NC2=O)CC1(C)C] | ¹H NMR (400 MHz, DMSO-d₆) δ 12.47 (s, 1H), 8.70 (d, J = 2.3 Hz, 1H), 8.21 (s, 1H), 7.87 (d, J = 8.7 Hz, 1H), 7.76 (s, 1H), 7.70 (dd, J = 8.5, 7.3 Hz, 1H), 7.63 (d, J = 8.0 Hz, 1H), 7.16 (d, J = 7.2 Hz, 1H), 6.94 (d, J = 8.4 Hz, 1H), 6.66 (d, J = 8.0 Hz, 1H), 6.49 (s, 1H), 5.41 (s, 1H), 4.23 (t, J = 2.9 Hz, 2H), 3.82 (t, J = 5.5 Hz, 2H), 3.28 (t, J = 8.4 Hz, 1H), 2.72 (t, J = 10.3 Hz, 1H), 2.47 (s, 2H), 2.36-2.29 (m, 1H), 2.02-1.92 (m, 2H), 1.85 (dd, J = 11.8, 5.3 Hz, 1H), 1.77 (d, J = 12.9 Hz, 1H), 1.66 (s, 3H), 1.57 (t, J = 12.4 Hz, 1H), 1.51 (s, 3H), 1.45 (d, J = 9.4 Hz, 1H), 1.28 (s, 9H). | | | |
| Compound 170 | 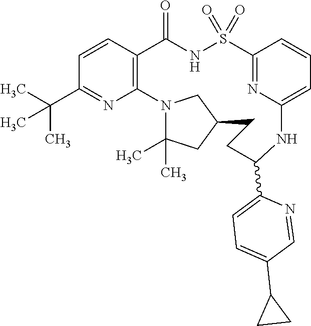 [SMILES: CC(C)(C)c1ccc2c(n1)N1C[C@@H](CCC(c3ccc(C4CC4)cn3)Nc3cccc(n3)S(=O)(=O)NC2=O)CC1(C)C] | ¹H NMR (400 MHz, DMSO-d₆) δ 12.44 (s, 1H), 8.63 (s, 1H), 8.01 (s, 1H), 7.92 (d, J = 8.6 Hz, 1H), 7.83 (s, 1H), 7.72 (dd, J = 8.4, 7.2 Hz, 1H), 7.64 (d, J = 8.0 Hz, 1H), 7.19 (d, J = 7.2 Hz, 1H), 6.94 (d, J = 8.4 Hz, 1H), 6.66 (d, J = 8.0 Hz, 1H), 5.43 (d, J = 7.2 Hz, 1H), 3.31 (t, J = 8.3 Hz, 1H), 2.68 (t, J = 10.2 Hz, 1H), 2.36 (s, 1H), 2.13 (d, J = 4.7 Hz, 1H), 1.96 (d, J = 5.7 Hz, 2H), 1.84 (dd, J = 11.8, 5.2 Hz, 1H), 1.76 (d, J = 14.5 Hz, 1H), 1.67 (s, 3H), 1.56 (t, J = 12.4 Hz, 1H), 1.50 (s, 3H), 1.48-1.39 (m, 1H), 1.28 (s, 9H), 1.13- 1.04 (m, 2H), 0.92-0.81 (m, 2H). | | | |
| Compound 136 | 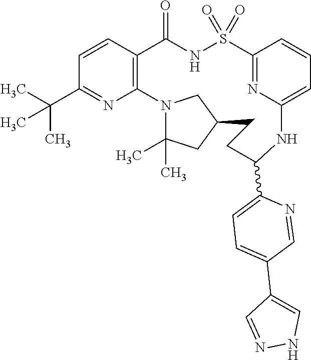 [SMILES: CC(C)(C)c1ccc2c(n1)N1C[C@@H](CCC(c3ccc(-c4cn[nH]c4)cn3)Nc3cccc(n3)S(=O)(=O)NC2=O)CC1(C)C] | ¹H NMR (400 MHz, DMSO-d₆) δ 12.47 (s, 1H), 9.00 (d, J = 2.1 Hz, 1H), 8.57 (s, 1H), 8.34 (s, 2H), 7.95 (t, J = 9.1 Hz, 2H), 7.72 (dd, J = 8.5, 7.3 Hz, 1H), 7.64 (d, J = 8.0 Hz, 1H), 7.19 (d, J = 7.2 Hz, 1H), 6.96 (d, J = 8.4 Hz, 1H), 6.66 (d, J = 8.0 Hz, 1H), 5.48 (d, J = 6.8 Hz, 1H), 3.35 (dd, J = 9.9, 6.9 Hz, 1H), 2.70 (t, J = 10.3 Hz, 1H), 2.54 (s, 1H), 2.39 (s, 1H), 2.02 (d, J = 9.7 Hz, 2H), 1.85 (dd, J = 11.7, 5.1 Hz, 1H), 1.78 (d, J = 14.6 Hz, 1H), 1.67 (s, 3H), 1.57 (t, J = 12.5 Hz, 1H), 1.51 (s, 3H), 1.46 (d, J = 14.2 Hz, 1H), 1.29 (s, 9H). | | | |
| Compound 130 | 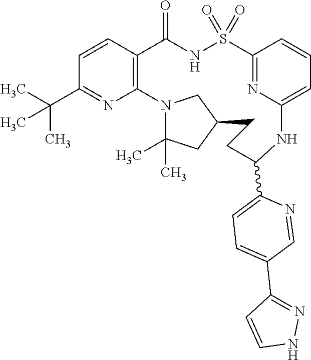 [SMILES: CC(C)(C)c1ccc2c(n1)N1C[C@@H](CCC(c3ccc(-c4cc[nH]n4)cn3)Nc3cccc(n3)S(=O)(=O)NC2=O)CC1(C)C] | ¹H NMR (400 MHz, DMSO-d₆) δ 12.51 (s, 1H), 9.04 (d, J = 2.2 Hz, 1H), 8.43 (s, 1H), 7.92-7.80 (m, 2H), 7.76 (s, 1H), 7.70 (dd, J = 8.5, 7.3 Hz, 1H), 7.63 (d, J = 8.0 Hz, 1H), 7.16 (d, J = 7.2 Hz, 1H), 6.94 (d, J = 8.4 Hz, 1H), 6.89 (d, J = 2.3 Hz, 1H), 6.66 (d, J = 8.0 Hz, 1H), 5.39 (t, J = 9.5 Hz, 1H), 3.27 (t, J = 8.5 Hz, 1H), 2.74 (t, J = 10.3 Hz, 1H), 2.75- 2.61 (m, 1H), 2.36-2.25 (m, 1H), 2.06-1.94 (m, 2H), 1.85 (dd, J = 11.8, 5.2 Hz, 1H), 1.77 (d, J = 13.8 Hz, 1H), 1.67 (s, 3H), 1.58 (t, J = 12.3 Hz, 1H), 1.51 (s, 3H), 1.50-1.42 (m, 1H), 1.29 (s, 9H). | | | |
| Compound 112 | 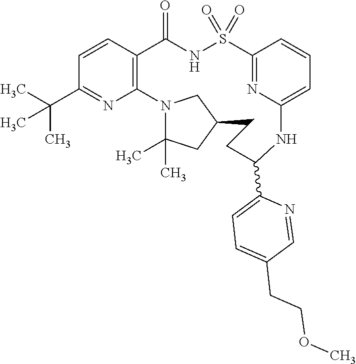 [SMILES: COCCc1ccc(C2CC[C@@H]3CN(c4nc(C(C)(C)C)ccc4C(=O)NS(=O)(=O)c4cccc(n4)N2)C(C)(C)C3)nc1] | ¹H NMR (400 MHz, DMSO-d₆) δ 12.37 (s, 1H), 8.59 (d, J = 2.0 Hz, 1H), 7.98 (d, J = 38.7 Hz, 1H), 7.85 (s, 2H), 7.66 (dt, J = 14.5, 8.0 Hz, 1H), 7.47 (d, J = 7.6 Hz, 1H), 7.27 (d, J = 7.2 Hz, 1H), 6.91 (d, J = 8.5 Hz, 1H), 6.63 (d, J = 7.9 Hz, 1H), 5.04 (s, 1H), 4.48 (q, J = 6.4 Hz, 1H), 3.16 (d, J = 1.3 Hz, 3H), 2.92 (s, 1H), 2.31 (s, 1H), 2.15 (s, 1H), 1.85 (dd, J = 11.9, 5.6 Hz, 1H), 1.83-1.72 (m, 1H), 1.69 (d, J = 22.7 Hz, 1H), 1.58 (s, 3H), 1.53 (s, 3H), 1.50 (d, J = 8.3 Hz, 1H), 1.38 (d, J = 6.5 Hz, 3H), 1.28 (s, 1H), 1.27 (s, 9H), 1.24-1.10 (m, 1H). | | | |
| Compound 103, hydrochloride salt | 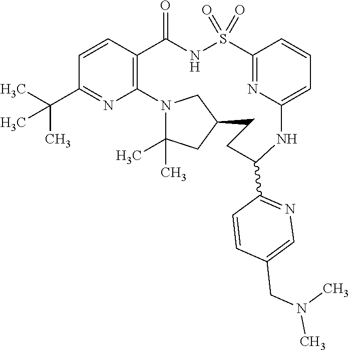 [SMILES: CN(C)Cc1ccc(C2CC[C@@H]3CN(c4nc(C(C)(C)C)ccc4C(=O)NS(=O)(=O)c4cccc(n4)N2)C(C)(C)C3)nc1] | ¹H NMR (400 MHz, DMSO-d₆) δ 12.29 (s, 1H), 10.70 (s, 1H), 8.73 (s, 1H), 8.02 (d, J = 8.2 Hz, 1H), 7.81 (s, 1H), 7.71 (d, J = 8.1 Hz, 1H), 7.64 (t, J = 7.9 Hz, 1H), 7.49 (s, 1H), 7.22 (d, J = 7.2 Hz, 1H), 6.88 (d, J = 8.5 Hz, 1H), 6.64 (d, J = 8.0 Hz, 1H), 5.04 (s, 1H), 4.31 (d, J = 5.2 Hz, 2H), 3.13-3.04 (m, 1H), 2.70 (d, J = 4.7 Hz, 6H), 2.69-2.63 (m, 1H), 2.32 (s, 1H), 2.22-2.11 (m, 1H), 1.86 (dd, J = 11.8, 5.8 Hz, 1H), 1.78-1.68 (m, 1H), 1.60 (s, 3H), 1.53 (s, 4H), 1.28 (s, 9H), 1.24-1.15 (m, 1H). | | | |
| Compound 98 | 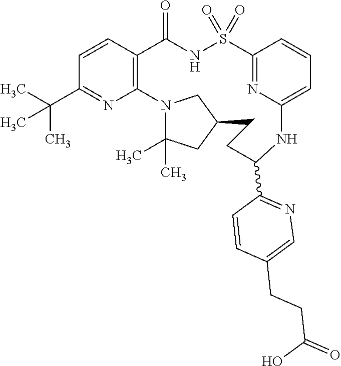 [SMILES: CC(C)(C)c1ccc2c(n1)N1C[C@@H](CCC(c3ccc(CCC(=O)O)cn3)Nc3cccc(n3)S(=O)(=O)NC2=O)CC1(C)C] | ¹H NMR (400 MHz, DMSO-d₆) δ 12.50 (s, 1H), 8.67 (s, 1H), 8.34 (s, 1H), 8.08 (s, 1H), 8.02-7.87 (m, 1H), 7.73 (t, J = 7.9 Hz, 1H), 7.44 (d, J = 8.0 Hz, 1H), 7.35 (d, J = 7.3 Hz, 1H), 6.95 (d, J = 8.4 Hz, 1H), 6.62 (d, J = 7.9 Hz, 1H), 4.83 (d, J = 9.1 Hz, 1H), 3.20 (s, 1H), 2.96 (t, J = 7.4 Hz, 2H), 2.67 (t, J = 7.4 Hz, 3H), 2.45-2.17 (m, 2H), 2.16-2.02 (m, 1H), 1.85 (dd, J = 11.8, 5.6 Hz, 1H), 1.80- 1.71 (m, 1H), 1.65 (d, J = 7.9 Hz, 1H), 1.59 (s, 1H), 1.56-1.51 (m, 4H), 1.44 (dd, J = 30.8, 12.9 Hz, 1H), 1.26 (s, 9H), 1.12 (d, J = 12.8 Hz, 1H) CO₂H (s, 1H) not observed. | | | |
| | | LCMS | | | |
| | | Retention | | | |
| Compound | | Time | Exact | | LCMS |
| Number | Structure | (min) | Mass | M + 1 | Method |
| Compound 168 | 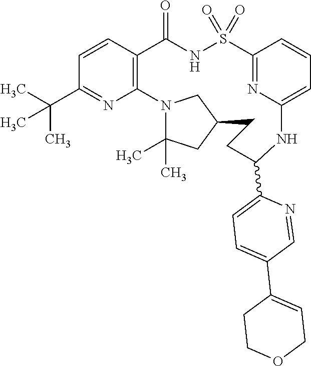 [SMILES: CC(C)(C)c1ccc2c(n1)N1C[C@@H](CCC(c3ccc(C4=CCOCC4)cn3)Nc3cccc(n3)S(=O)(=O)NC2=O)CC1(C)C] | 1.87 | 630.299 | 631.2 | LC method A |
| Compound 170 | 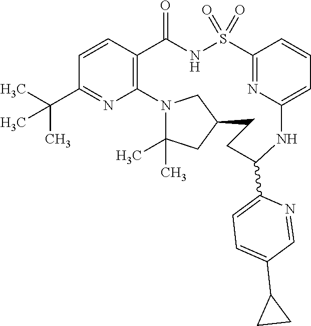 [SMILES: CC(C)(C)c1ccc2c(n1)N1C[C@@H](CCC(c3ccc(C4CC4)cn3)Nc3cccc(n3)S(=O)(=O)NC2=O)CC1(C)C] | 1.82 | 588.288 | 589.3 | LC method A |
| Compound 136 | 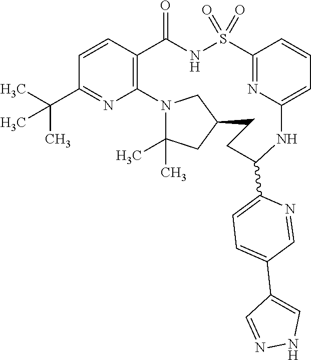 [SMILES: CC(C)(C)c1ccc2c(n1)N1C[C@@H](CCC(c3ccc(-c4cn[nH]c4)cn3)Nc3cccc(n3)S(=O)(=O)NC2=O)CC1(C)C] | 1.62 | 614.279 | 615.2 | LC method A |
| Compound 130 | 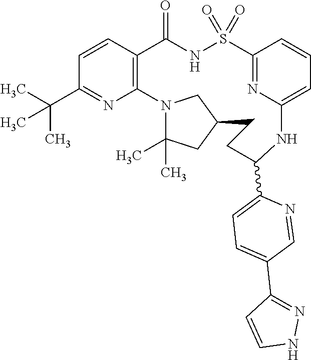 [SMILES: CC(C)(C)c1ccc2c(n1)N1C[C@@H](CCC(c3ccc(-c4cc[nH]n4)cn3)Nc3cccc(n3)S(=O)(=O)NC2=O)CC1(C)C] | 1.69 | 614.279 | 615.3 | LC method A |
| Compound 112 | 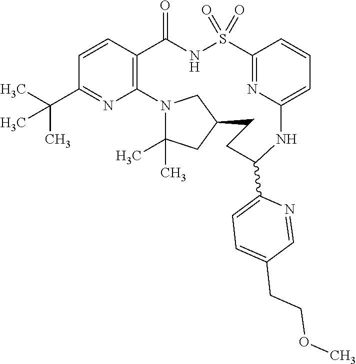 [SMILES: COCCc1ccc(C2CC[C@@H]3CN(c4nc(C(C)(C)C)ccc4C(=O)NS(=O)(=O)c4cccc(n4)N2)C(C)(C)C3)nc1] | 1.64 | 606.299 | 607.3 | LC method A |
| Compound 103, hydrochloride salt | 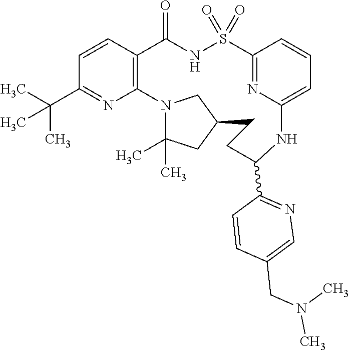 [SMILES: CN(C)Cc1ccc(C2CC[C@@H]3CN(c4nc(C(C)(C)C)ccc4C(=O)NS(=O)(=O)c4cccc(n4)N2)C(C)(C)C3)nc1] | 1.38 | 605.315 | 606.2 | LC method A |
| Compound 98 | 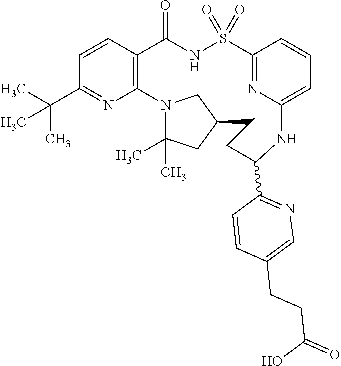 [SMILES: CC(C)(C)c1ccc2c(n1)N1C[C@@H](CCC(c3ccc(CCC(=O)O)cn3)Nc3cccc(n3)S(=O)(=O)NC2=O)CC1(C)C] | 1.45 | 620.278 | 621.2 | LC method A |
| Compound 146 | 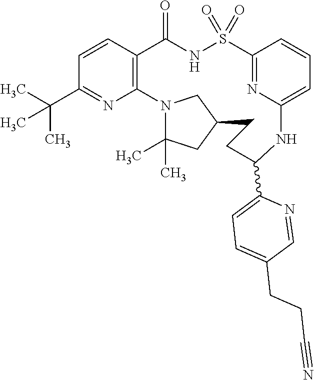 [SMILES: CC(C)(C)c1ccc2c(n1)N1C[C@@H](CCC(c3ccc(CCC#N)cn3)Nc3cccc(n3)S(=O)(=O)NC2=O)CC1(C)C] | 1.66 | 601.284 | 602.2 | LC method A |
| Compound 114, hydrochloride salt | 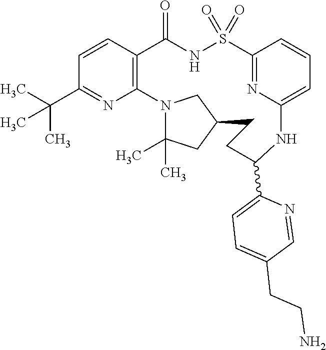 [SMILES: CC(C)(C)c1ccc2c(n1)N1C[C@@H](CCC(c3ccc(CCN)cn3)Nc3cccc(n3)S(=O)(=O)NC2=O)CC1(C)C] | 1.42 | 591.299 | 592.2 | LC method A |
| Compound 156 | 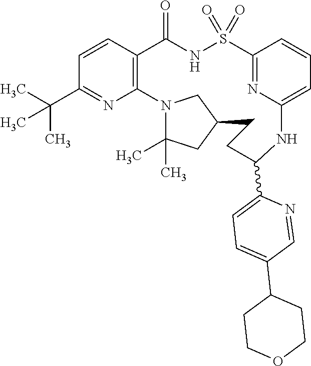 [SMILES: CC(C)(C)c1ccc2c(n1)N1C[C@@H](CCC(c3ccc(C4CCOCC4)cn3)Nc3cccc(n3)S(=O)(=O)NC2=O)CC1(C)C] | 1.58 | 632.314 | 633.4 | LC method A |
| Compound 166, hydrochloride salt | 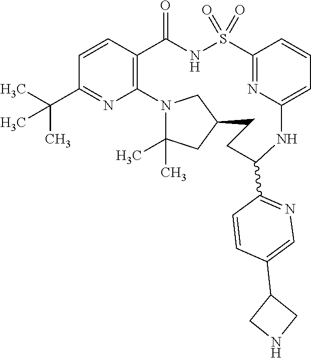 [SMILES: CC(C)(C)c1ccc2c(n1)N1C[C@@H](CCC(c3ccc(C4CNC4)cn3)Nc3cccc(n3)S(=O)(=O)NC2=O)CC1(C)C] | 1.49 | 603.299 | 604.2 | LC method A |

### Example 32: Preparation of (14S)-8-tert-butyl-12,12-dimethyl-17-[5-(pyrrolidin-1-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione, Compound 164

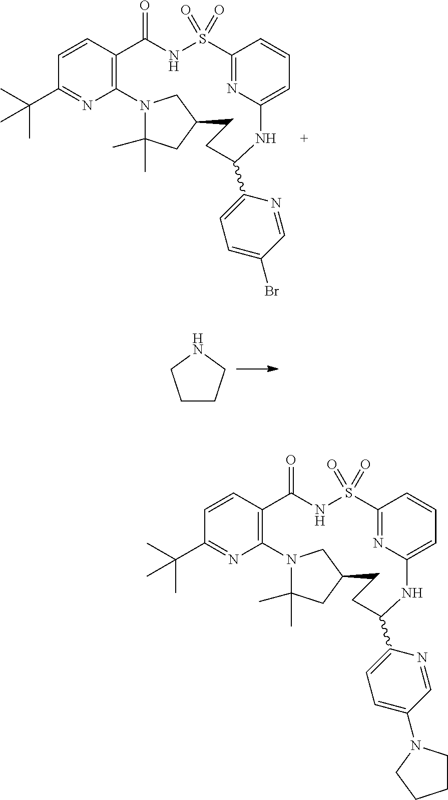 [SMILES: CC(C)(C)c1ccc2c(n1)N1C[C@@H](CCC(c3ccc(Br)cn3)Nc3cccc(n3)S(=O)(=O)NC2=O)CC1(C)C ; C1CCNC1 ; CC(C)(C)c1ccc2c(n1)N1C[C@@H](CCC(c3ccc(N4CCCC4)cn3)Nc3cccc(n3)S(=O)(=O)NC2=O)CC1(C)C]

In a microwave vial (14S)-17-(5-bromopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 172 (diastereomer 1), 30 mg, 0.0468 mmol) was dissolved in dioxane (650 μL) and DMF (600 μL) and to the mixture was added cesium carbonate (81 mg, 0.249 mmol) followed by pyrrolidine (25 μL, 0.299 mmol), Xantphos (8.6 mg, 0.0149 mmol) and Pd₂(dba)₃ (11 mg, 0.0120 mmol). The mixture was purged with nitrogen, sealed and heated at 130° C. for 18 h. Cooled to room temperature and the mixture was filtered, and purified by reverse-phase preparative chromatography utilizing a C₁₈ column and 20-80% gradient over 15 min of acetonitrile in water (+5 mM HCl) using a 15 minute run to afford (14S)-8-tert-butyl-12,12-dimethyl-17-[5-(pyrrolidin-1-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione (19.22 mg, 64%). ESI-MS m/z calc. 617.3148, found 618.2 (M+1)⁺; Retention time: 1.84 minutes (LC method A).

### Example 33: Preparation of (14S)-17-(5-aminopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 64

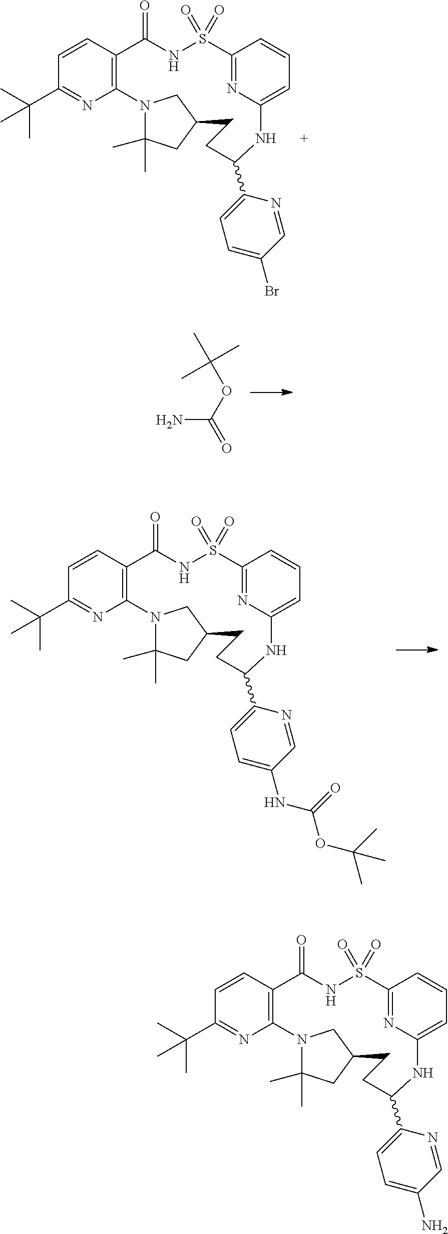

Stage 1: In a microwave vial (14S)-17-(5-bromopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 172 (diastereomer 1), 30 mg, 0.04780 mmol) was dissolved in DMF (750 μL) and to the mixture was added cesium carbonate (95 mg, 0.2916 mmol) followed by tert-butyl carbamate (30 mg, 0.2561 mmol), Xantphos (9.5 mg, 0.0164 mmol) and Pd₂(dba)₃ (11.5 mg, 0.0126 mmol). The mixture was purged with nitrogen, sealed and heated at 110° C. for 20 h. Cooled to room temperature and the mixture was filtered, and purified by reverse-phase preparative chromatography utilizing a C₁₈ column and a 1-70% gradient over 30 min of acetonitrile in water (+5 mM HCl) to afford tert-butyl N-{6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-3-yl}carbamate. ESI-MS m/z calc. 663.3203, found 664.2 (M+1)⁺; Retention time: 1.79 minutes (LC method A).

Stage 2: The mixture from Step 1 was dissolved in DCM (750 μL) and to it was added TFA (200 μL, 2.596 mmol) and the mixture was stirred for 30 min. Concentrated sample and purified by reverse-phase preparative chromatography utilizing a C₁₈ column and a 1-50% gradient over 30 min of acetonitrile in water (+5 mM HCl) to afford (145)-17-(5-aminopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (8.5 mg, 30%). ESI-MS m/z calc. 563.2679, found 564.2 (M+1)⁺; Retention time: 1.53 minutes (LC method A).

### Example 34: Preparation of methyl 3-{6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-3-yl}propanoate, Compound 91

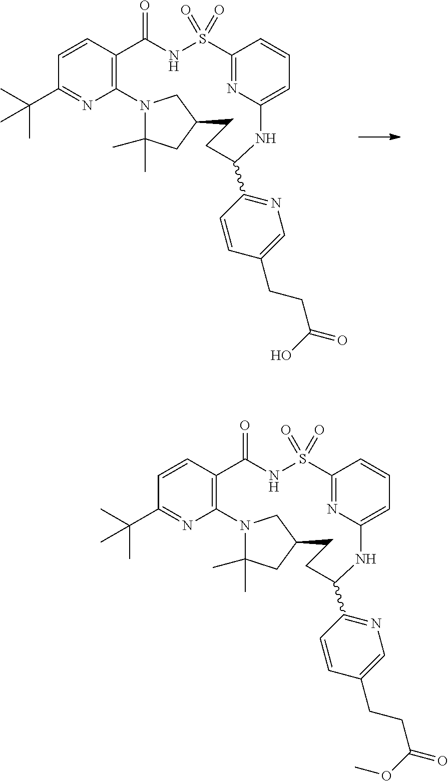 [SMILES: CC(C)(C)c1ccc2c(n1)N1C[C@@H](CCC(c3ccc(CCC(=O)O)cn3)Nc3cccc(n3)S(=O)(=O)NC2=O)CC1(C)C ; COC(=O)CCc1ccc(C2CC[C@@H]3CN(c4nc(C(C)(C)C)ccc4C(=O)NS(=O)(=O)c4cccc(n4)N2)C(C)(C)C3)nc1]

3-{6-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-3-yl}propanoic acid (Compound 98, 15 mg, 0.02392 mmol) was dissolved in MeOH (500 μL) and the solution was cooled to 0° C. To the mixture was added thionyl chloride (20 μL, 0.2742 mmol) and then the cooling bath was removed and the reaction mixture was allowed to stir at rt for 4 h. The mixture was purified by reverse-phase preparative chromatography utilizing a C₁₈ column and a 1-70% over 30 min gradient of acetonitrile in water (+5 mM HCl) to afford methyl 3-{6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-3-yl}propanoate (10.1 mg, 63%). ¹H NMR (400 MHz, DMSO-d₆) δ 12.43 (s, 1H), 8.63 (s, 1H), 8.20 (s, 1H), 8.01-7.76 (m, 2H), 7.70 (t, J=8.0 Hz, 1H), 7.45 (d, J=7.8 Hz, 1H), 7.31 (d, J=6.5 Hz, 1H), 6.96-6.89 (m, 1H), 6.63 (t, J=7.2 Hz, 1H), 5.02 (s, 1H), 3.58 (s, 2H), 2.94 (dd, J=11.6, 7.2 Hz, 3H), 2.74 (t, J=7.5 Hz, 2H), 2.64 (d, J=7.5 Hz, 1H), 2.34-2.21 (m, 1H), 2.10 (s, 1H), 1.85 (dd, J=11.8, 5.7 Hz, 1H), 1.80-1.71 (m, 1H), 1.65 (t, J=9.5 Hz, 1H), 1.55 (d, J=8.7 Hz, 6H), 1.44 (dd, J=34.1, 14.5 Hz, 2H), 1.26 (d, J=2.4 Hz, 9H), 1.22-1.09 (m, 1H). ESI-MS m/z calc. 634.29376, found 635.2 (M+1)⁺; Retention time: 1.58 minutes (LC method A).

### Example 35: Preparation of (14S)-17-[5-(3-aminopropyl)pyridin-2-yl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 138

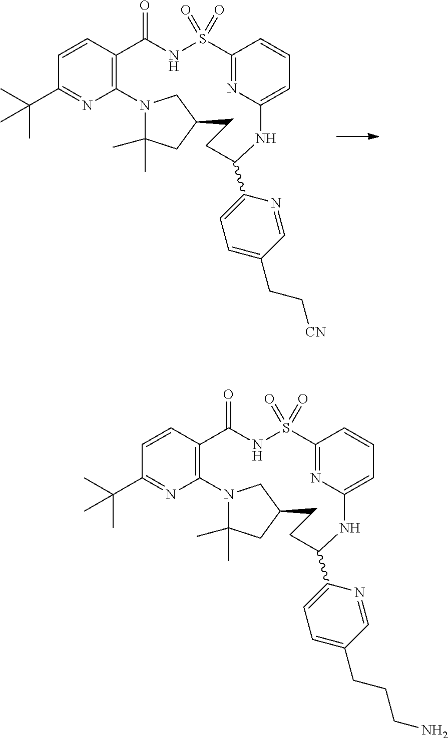 [SMILES: CC(C)(C)c1ccc2c(n1)N1C[C@@H](CCC(c3ccc(CCC#N)cn3)Nc3cccc(n3)S(=O)(=O)NC2=O)CC1(C)C ; CC(C)(C)c1ccc2c(n1)N1C[C@@H](CCC(c3ccc(CCCN)cn3)Nc3cccc(n3)S(=O)(=O)NC2=O)CC1(C)C]

In a 4 mL vial 3-{6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-3-yl}propanenitrile (Compound 146, 13.0 mg, 0.02139 mmol) was dissolved in EtOH (1.2 mL) and purged with nitrogen for 5 minutes. To the mixture was added raney nickel (4 mg of 50% w/w, 0.03408 mmol) followed by a hydrogen balloon. The mixture was sealed and heated to 60° C. for 14 h. Cooled to room temperature and the hydrogen balloon was removed and the vessel was quickly degassed with nitrogen. Added 2 drops of conc. HCl, stirred 1 minute then filtered eluting with methanol. The filtrate was concentrated, dissolved in minimal DMSO then the mixture was filtered, and purified by reverse-phase preparative chromatography utilizing a C₁₈ column and a 10-60% gradient over 15 min of acetonitrile in water (+5 mM HCl) to afford (14S)-17-[5-(3-aminopropyl)pyridin-2-yl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (8.2 mg, 59%). ESI-MS m/z calc. 605.3148, found 606.4 (M+1)⁺; Retention time: 1.38 minutes (LC method A).

### Example 36: Preparation of analogs related to 6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-3-carbonitrile (Compound 205)

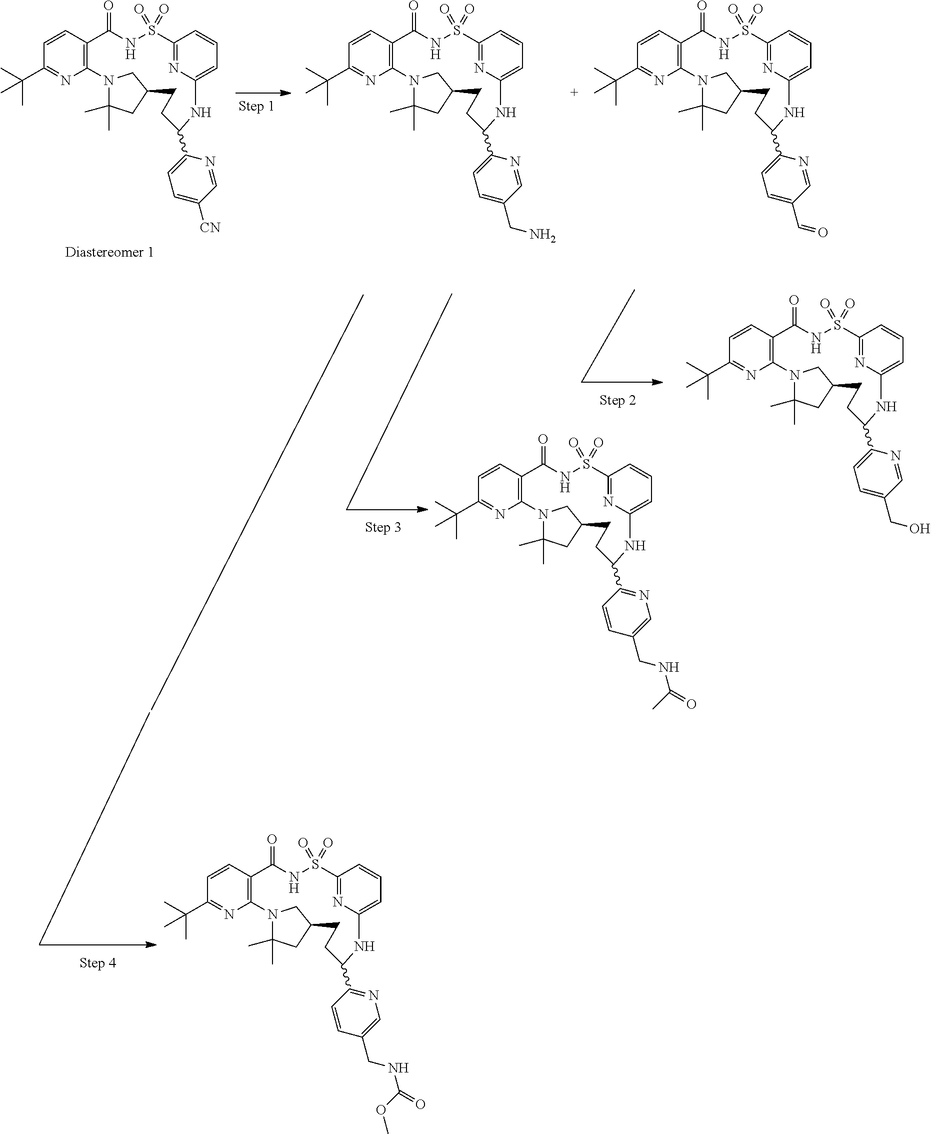

### Step 1: (14S)-17-[5-(Aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 197, and 6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-3-carbaldehyde

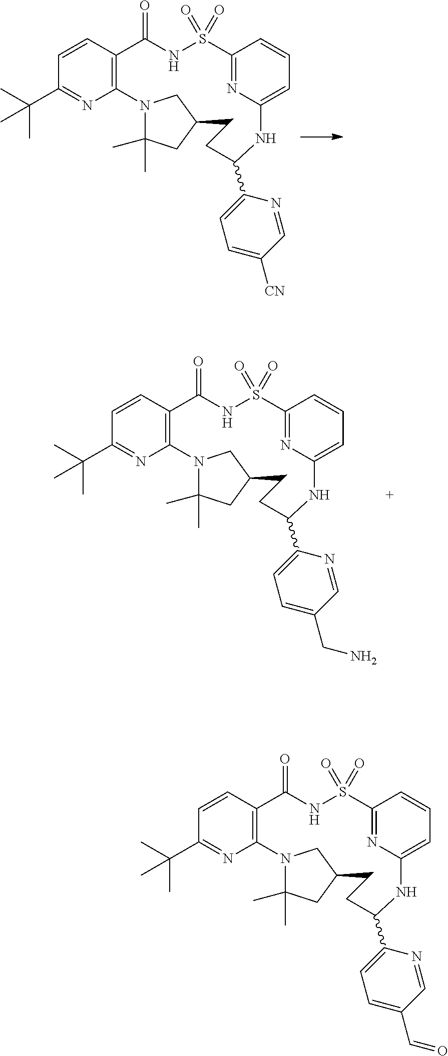 [SMILES: CC(C)(C)c1ccc2c(n1)N1C[C@@H](CCC(c3ccc(C#N)cn3)Nc3cccc(n3)S(=O)(=O)NC2=O)CC1(C)C ; CC(C)(C)c1ccc2c(n1)N1C[C@@H](CCC(c3ccc(CN)cn3)Nc3cccc(n3)S(=O)(=O)NC2=O)CC1(C)C ; CC(C)(C)c1ccc2c(n1)N1C[C@@H](CCC(c3ccc(C=O)cn3)Nc3cccc(n3)S(=O)(=O)NC2=O)CC1(C)C]

In a microwave vial 6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-3-carbonitrile (Compound 205 (diastereomer 1), 88 mg, 0.1534 mmol) was dissolved in EtOH (4 mL) and purged with nitrogen for 5 min. To the mixture was added raney nickel (35 mg of 50% w/w, 0.2982 mmol) followed by hydrogen balloon. The mixture was sealed and heated to 60° C. for 4 h. The mixture was filtered and purified by reverse-phase preparative chromatography utilizing a C₁₈ column and a 1-70% gradient over 15 min of acetonitrile in water (+5 mM HCl) to afford (14S)-17-[5-(aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (10.7 mg, 11%). ESI-MS m/z calc. 577.2835, found 578.2 (M+1)⁺; Retention time: 1.34 minutes (LC method A).

Also isolated 6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-3-carbaldehyde (4.2 mg, 5%). ESI-MS m/z calc. 576.2519, found 577.2 (M+1)⁺; Retention time: 1.95 minutes (LC method A).

### Step 2: (14S)-8-tert-butyl-17-[5-(hydroxymethyl)pyridin-2-yl]-12,12-dimethyl-R⁶-thia-3,9,11,18,23-pentaazatetracyclo[17.3.1.111,14.05,10]tetracosa-1(23),5(10),6,8,19,21-hexaene-2,2,4-trione, Compound 79

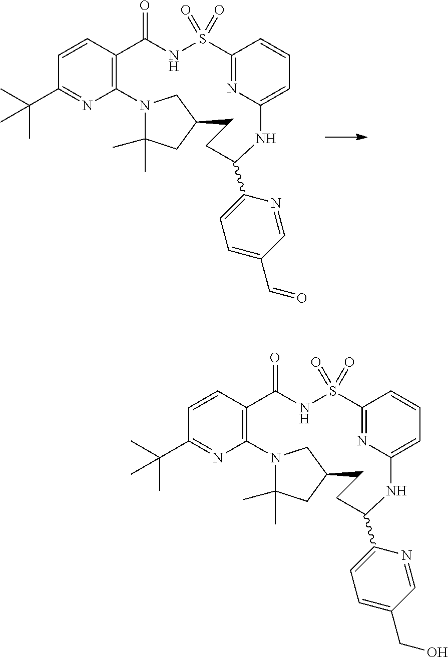 [SMILES: CC(C)(C)c1ccc2c(n1)N1C[C@@H](CCC(c3ccc(C=O)cn3)Nc3cccc(n3)S(=O)(=O)NC2=O)CC1(C)C ; CC(C)(C)c1ccc2c(n1)N1C[C@@H](CCC(c3ccc(CO)cn3)Nc3cccc(n3)S(=O)(=O)NC2=O)CC1(C)C]

To a stirring solution of 6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-3-carbaldehyde (originating from Compound 205 (diastereomer 1), 4.5 mg, 0.0078 mmol) in methanol (250 μL) under nitrogen was added NaBH₄ (2.0 mg, 0.0529 mmol) with minimal effervescence. After complete addition the reaction was a solution, and after stirring for 5 minutes the mixture was filtered, and purified by reverse-phase preparative chromatography utilizing a C₁₈ column and a 1-70% gradient over 15 min of acetonitrile in water (+5 mM HCl) to afford a white solid as (14S)-8-tert-butyl-17-[5-(hydroxymethyl)pyridin-2-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (1.5 mg, 33%). ESI-MS m/z calc. 578.2675, found 579.2 (M+1)⁺; Retention time: 1.54 minutes (LC method A).

### Step 3: N-({6-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-3-yl}methyl)acetamide, Compound 72

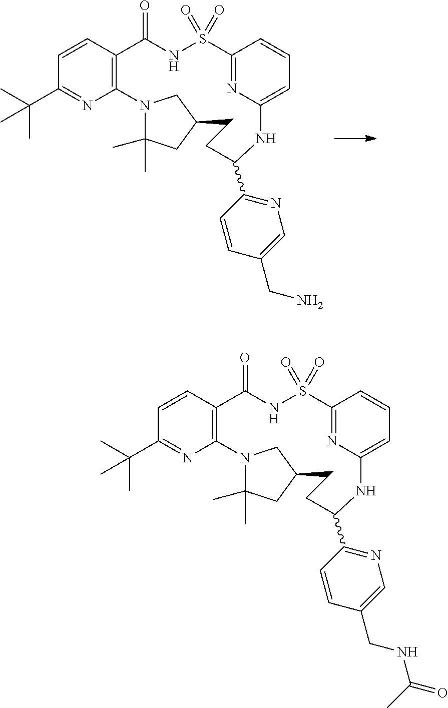 [SMILES: CC(C)(C)c1ccc2c(n1)N1C[C@@H](CCC(c3ccc(CN)cn3)Nc3cccc(n3)S(=O)(=O)NC2=O)CC1(C)C ; CC(=O)NCc1ccc(C2CC[C@@H]3CN(c4nc(C(C)(C)C)ccc4C(=O)NS(=O)(=O)c4cccc(n4)N2)C(C)(C)C3)nc1]

(14S)-17-[5-(aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (Compound 197 (diastereomer 1), 26 mg, 0.04233 mmol) peak 1 (more polar) was dissolved in anhydrous DCM (1.0 mL) under a nitrogen atmosphere and to it was added TEA (25 μL, 0.179 mmol) and the reaction was stirred for 5 minutes upon which acetic anhydride (5 μL, 0.053 mmol) was added. The reaction was allowed to stir for 18 h at room temp. Added additional acetic anhydride (5 μL, 0.05299 mmol) and the reaction was allowed to stir for 3 h at room temp. The mixture was filtered, and purified by reverse-phase preparative chromatography utilizing a C₁₈ column and a 1-70% gradient over 30 min of acetonitrile in water (+5 mM HCl) to afford a white solid as N-({6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-3-yl}methyl)acetamide (20.5 mg, 77%). ¹H NMR (400 MHz, DMSO-d₆) δ 12.49 (s, 1H), 8.65-8.58 (m, 2H), 8.32-8.19 (m, 1H), 8.05 (d, J=18.9 Hz, 2H), 7.72 (t, J=7.9 Hz, 1H), 7.45 (d, J=7.9 Hz, 1H), 7.34 (d, J=7.3 Hz, 1H), 6.95 (d, J=8.5 Hz, 1H), 6.62 (d, J=7.9 Hz, 1H), 5.13 (s, 1H), 4.37 (d, J=5.8 Hz, 2H), 3.21 (s, 1H), 2.72 (d, J=5.3 Hz, 1H), 2.35-2.25 (m, 1H), 2.11 (s, 1H), 1.88 (s, 3H), 1.84 (t, J=5.8 Hz, 1H), 1.81-1.72 (m, 1H), 1.61 (d, J=20.0 Hz, 1H), 1.54 (s, 5H), 1.47 (d, J=11.6 Hz, 1H), 1.26 (s, 10H), 1.22-1.10 (m, 1H). ESI-MS m/z calc. 619.29407, found 620.8 (M+1)⁺; Retention time: 1.44 minutes (LC method A).

### Step 4: Methyl N-({6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-3-yl}methyl)carbamate, Compound 89

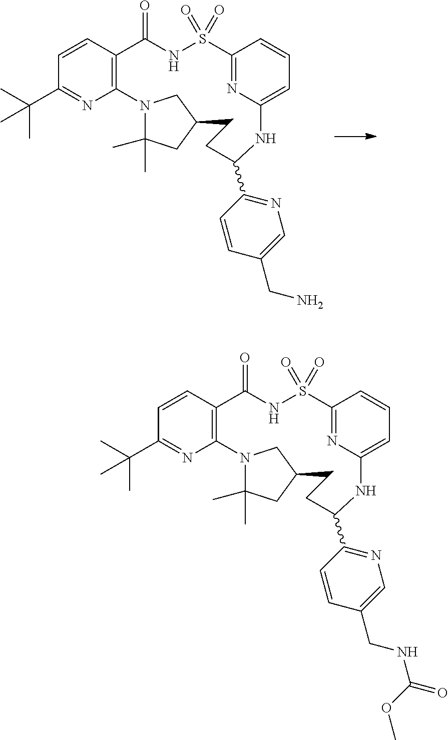 [SMILES: CC(C)(C)c1ccc2c(n1)N1C[C@@H](CCC(c3ccc(CN)cn3)Nc3cccc(n3)S(=O)(=O)NC2=O)CC1(C)C ; COC(=O)NCc1ccc(C2CC[C@@H]3CN(c4nc(C(C)(C)C)ccc4C(=O)NS(=O)(=O)c4cccc(n4)N2)C(C)(C)C3)nc1]

(14S)-17-[5-(Aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (Compound 197 (diastereomer 1), 10.7 mg, 0.01742 mmol) was dissolved in anhydrous DCM (750 μL) under a nitrogen atmosphere and to it was added TEA (15 μL, 0.1076 mmol) and the reaction was stirred for 5 minutes upon which methylchloroformate (2 μL, 0.02588 mmol) was added dropwise. The reaction was allowed to stir for 4 h at rt. The mixture was filtered, and purified by reverse-phase preparative chromatography utilizing a C₁₈ column and a 1-70% gradient over 30 min of acetonitrile in water (+5 mM HCl) to afford a white solid as methyl N-({6-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-3-yl}methyl)carbamate (2.1 mg, 18%). ESI-MS m/z calc. 635.289, found 636.4 (M+1)⁺; Retention time: 1.52 minutes (LC method A).

### Example 37: Preparation of (14S)-8-tert-butyl-12,12-dimethyl-17-[5-(oxetan-3-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione, Compound 29

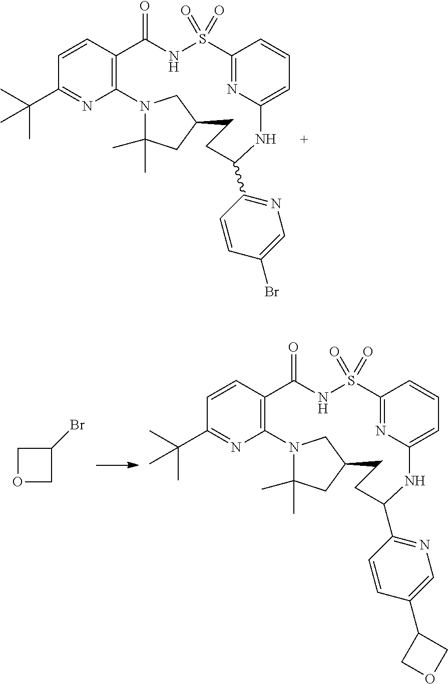 [SMILES: CC(C)(C)c1ccc2c(n1)N1C[C@@H](CCC(c3ccc(Br)cn3)Nc3cccc(n3)S(=O)(=O)NC2=O)CC1(C)C ; BrC1COC1 ; CC(C)(C)c1ccc2c(n1)N1C[C@@H](CCC(c3ccc(C4COC4)cn3)Nc3cccc(n3)S(=O)(=O)NC2=O)CC1(C)C]

4-tert-Butyl-2-(4-tert-butyl-2-pyridyl)pyridine (2 mg, 0.007452 mmol), [Ir{dF(CF₃)ppy}2(dtbpy)]PF₆ (0.8 mg, 7.131E-4 mmol) and nickel(II) chloride,1,2-dimethoxyethane complex (1 mg, 0.004780 mmol) were added to a 1 dram vial. It was capped with a septum and purged 3 times with vacuum/nitrogen backfilling cycles. DME (300 μL) and 2,6-dimethylpyridine (25 μL, 0.2159 mmol) were added and the reaction was stirred for 10 minutes to form the ligated nickel (turned blue). (14S)-17-(5-Bromopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 172 (diastereomer 1)) (30 mg, 0.04780 mmol) was dissolved in DME (600 μL). 3-Bromooxetane (20 mg, 0.1460 mmol) and bis(trimethylsilyl)silyl-trimethyl-silane (45 μL, 0.1459 mmol) were added. The reaction was irradiated for 2 h in a Merck Photoreactor (100% power, 4700 rpm fan, 1700 rpm stirring). And then it was diluted with DCM and washed with a saturated ammonium chloride solution. Volatiles were evaporated before purifying the crude residue on silica gel (preparative TLC 100% EtOAc mobile phase rt 0.6). Yielded (14S)-8-tert-butyl-12,12-dimethyl-17-[5-(oxetan-3-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione (9.3 mg, 32%). ESI-MS m/z calc. 604.2832, found 605.5 (M+1)⁺; Retention time: 1.49 minutes as a white solid (LC method A).

### Example 38: Preparation of analogs of (14S)-17-(5-bromopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 171 (diastereomer 2)

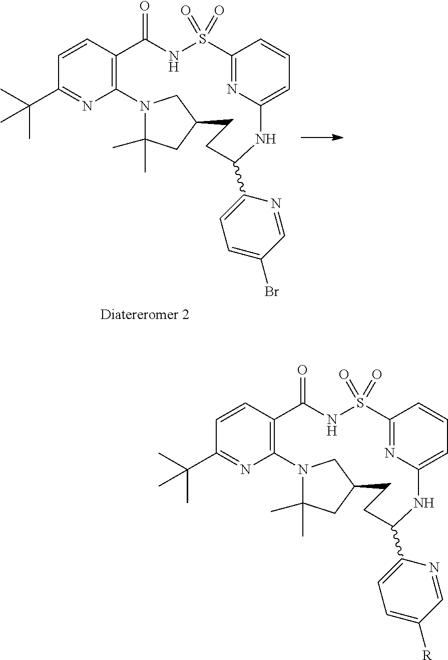

The compounds in the following tables were prepared in a manner analogous to that described above using (14S)-17-(5-bromopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 171 (diastereomer 2), as a starting material.

Compound 39 and Compound 21 were prepared in a manner analogous to the photoredox based preparation of Compound 29, described above, using 3-bromooxetane and 3-bromo-1,1-difluoro-cyclobutane as starting materials.

| | | LCMS | | | |
|---|---|---|---|---|---|
| | | Retention | | | |
| Compound | | Time | Exact | | LCMS |
| Number | Structure | (min) | Mass | M + 1 | Method |
| Compound 169 | 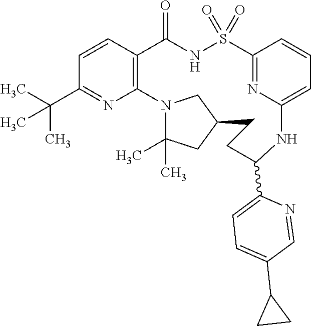 [SMILES: CC(C)(C)c1ccc2c(n1)N1C[C@@H](CCC(c3ccc(C4CC4)cn3)Nc3cccc(n3)S(=O)(=O)NC2=O)CC1(C)C] | 1.73 | 588.288 | 589.2 | LC method A |
| Compound 167 | 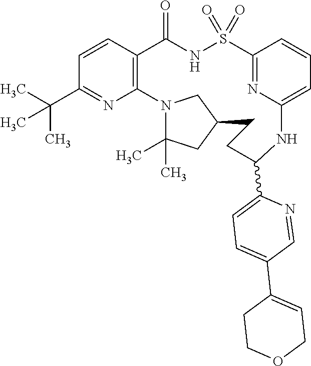 [SMILES: CC(C)(C)c1ccc2c(n1)N1C[C@@H](CCC(c3ccc(C4=CCOCC4)cn3)Nc3cccc(n3)S(=O)(=O)NC2=O)CC1(C)C] | 1.73 | 630.299 | 631.2 | LC method A |
| Compound 165, hydrochloride salt | 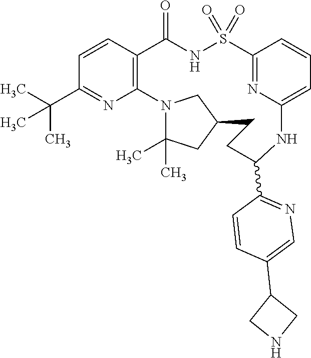 [SMILES: CC(C)(C)c1ccc2c(n1)N1C[C@@H](CCC(c3ccc(C4CNC4)cn3)Nc3cccc(n3)S(=O)(=O)NC2=O)CC1(C)C] | 1.32 | 603.299 | 604.2 | LC method A |
| Compound 163 | 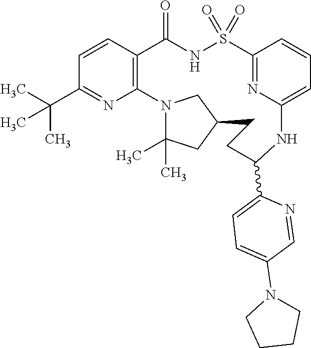 [SMILES: CC(C)(C)c1ccc2c(n1)N1C[C@@H](CCC(c3ccc(N4CCCC4)cn3)Nc3cccc(n3)S(=O)(=O)NC2=O)CC1(C)C] | 1.8 | 617.315 | 618.3 | LC method A |
| Compound 155 | 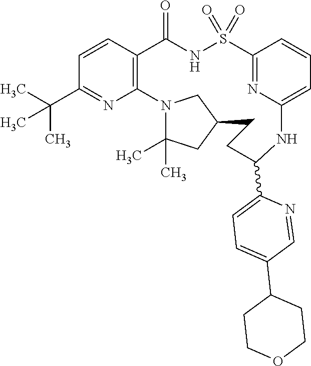 [SMILES: CC(C)(C)c1ccc2c(n1)N1C[C@@H](CCC(c3ccc(C4CCOCC4)cn3)Nc3cccc(n3)S(=O)(=O)NC2=O)CC1(C)C] | 1.67 | 632.314 | 633.8 | LC method A |
| Compound 153, hydrochloride salt | 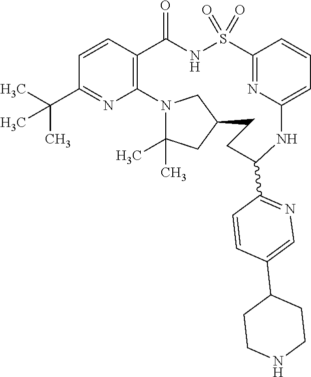 [SMILES: CC(C)(C)c1ccc2c(n1)N1C[C@@H](CCC(c3ccc(C4CCNCC4)cn3)Nc3cccc(n3)S(=O)(=O)NC2=O)CC1(C)C] | 1.26 | 631.33 | 632.2 | LC method A |
| Compound 145 | 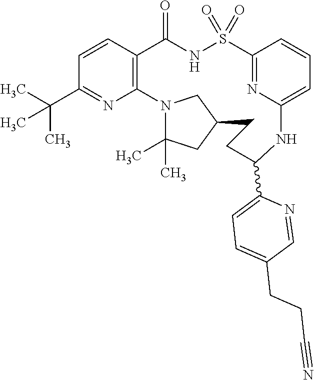 [SMILES: CC(C)(C)c1ccc2c(n1)N1C[C@@H](CCC(c3ccc(CCC#N)cn3)Nc3cccc(n3)S(=O)(=O)NC2=O)CC1(C)C] | 1.56 | 601.284 | 602.2 | LC method A |
| Compound 137, hydrochloride salt | 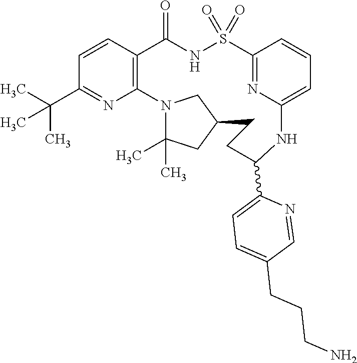 [SMILES: CC(C)(C)c1ccc2c(n1)N1C[C@@H](CCC(c3ccc(CCCN)cn3)Nc3cccc(n3)S(=O)(=O)NC2=O)CC1(C)C] | 1.22 | 605.315 | 606.2 | LC method A |
| Compound 135 | 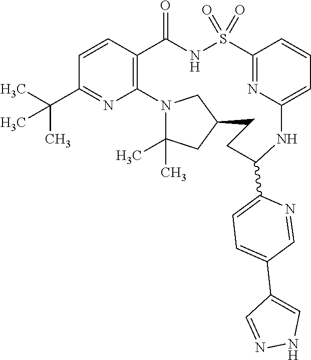 [SMILES: CC(C)(C)c1ccc2c(n1)N1C[C@@H](CCC(c3ccc(-c4cn[nH]c4)cn3)Nc3cccc(n3)S(=O)(=O)NC2=O)CC1(C)C] | 1.5 | 614.279 | 615.3 | LC method A |
| Compound 129 | 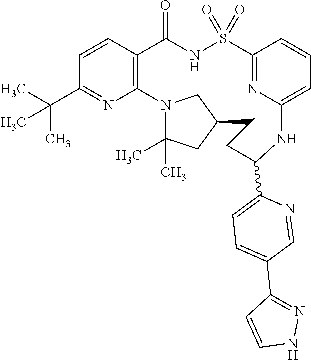 [SMILES: CC(C)(C)c1ccc2c(n1)N1C[C@@H](CCC(c3ccc(-c4cc[nH]n4)cn3)Nc3cccc(n3)S(=O)(=O)NC2=O)CC1(C)C] | 1.58 | 614.279 | 615.3 | LC method A |
| Compound 113, hydrochloride salt | 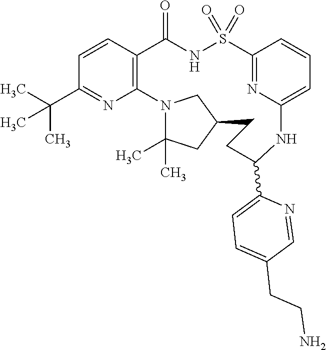 [SMILES: CC(C)(C)c1ccc2c(n1)N1C[C@@H](CCC(c3ccc(CCN)cn3)Nc3cccc(n3)S(=O)(=O)NC2=O)CC1(C)C] | 1.28 | 591.299 | 592.2 | LC method A |
| Compound 111 | 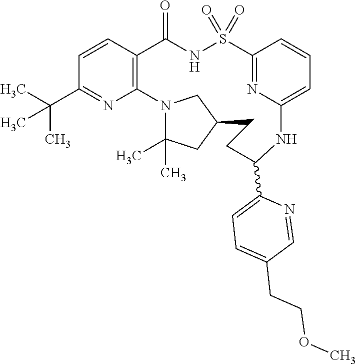 [SMILES: COCCc1ccc(C2CC[C@@H]3CN(c4nc(C(C)(C)C)ccc4C(=O)NS(=O)(=O)c4cccc(n4)N2)C(C)(C)C3)nc1] | 1.55 | 606.299 | 607.3 | LC method A |
| Compound 102, hydrochloride salt | 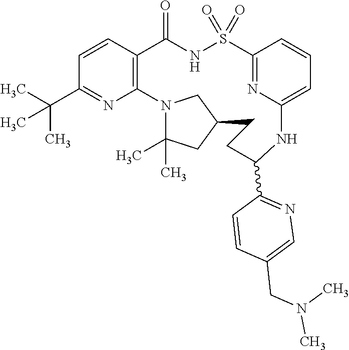 [SMILES: CN(C)Cc1ccc(C2CC[C@@H]3CN(c4nc(C(C)(C)C)ccc4C(=O)NS(=O)(=O)c4cccc(n4)N2)C(C)(C)C3)nc1] | 1.53 | 605.315 | 606.2 | LC method A |
| Compound 97 | 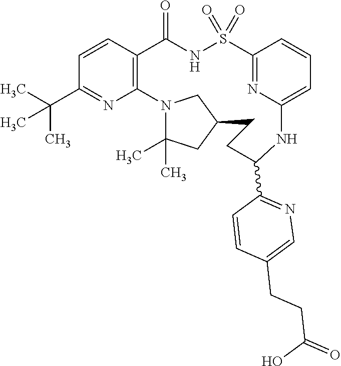 [SMILES: CC(C)(C)c1ccc2c(n1)N1C[C@@H](CCC(c3ccc(CCC(=O)O)cn3)Nc3cccc(n3)S(=O)(=O)NC2=O)CC1(C)C] | 1.56 | 620.278 | 621.2 | LC method A |
| Compound 90 | 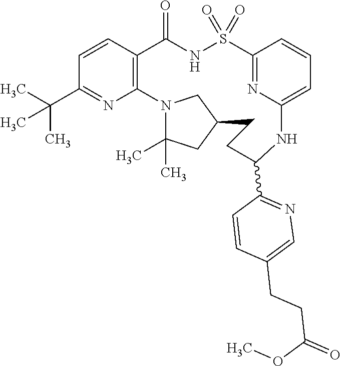 [SMILES: COC(=O)CCc1ccc(C2CC[C@@H]3CN(c4nc(C(C)(C)C)ccc4C(=O)NS(=O)(=O)c4cccc(n4)N2)C(C)(C)C3)nc1] | 1.68 | 634.294 | 635.2 | LC method A |
| Compound 88 | 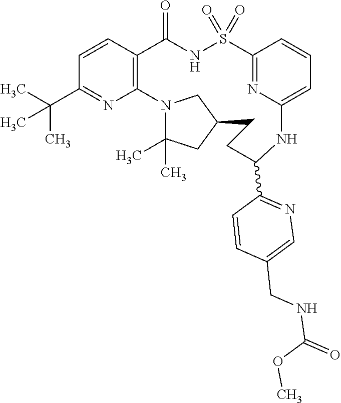 [SMILES: COC(=O)NCc1ccc(C2CC[C@@H]3CN(c4nc(C(C)(C)C)ccc4C(=O)NS(=O)(=O)c4cccc(n4)N2)C(C)(C)C3)nc1] | 1.63 | 635.289 | 636.2 | LC method A |
| Compound 78 | 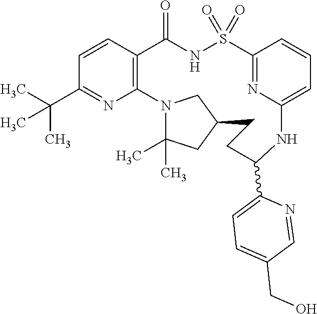 [SMILES: CC(C)(C)c1ccc2c(n1)N1C[C@@H](CCC(c3ccc(CO)cn3)Nc3cccc(n3)S(=O)(=O)NC2=O)CC1(C)C] | 1.54 | 578.268 | 579.2 | LC method A |
| Compoun 71 | 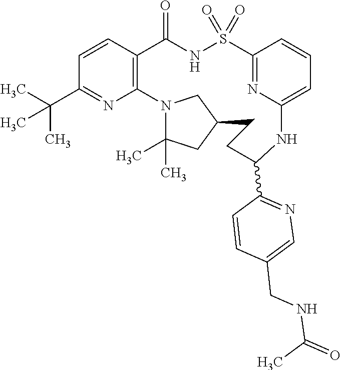 [SMILES: CC(=O)NCc1ccc(C2CC[C@@H]3CN(c4nc(C(C)(C)C)ccc4C(=O)NS(=O)(=O)c4cccc(n4)N2)C(C)(C)C3)nc1] | 1.56 | 619.294 | 620.3 | LC method A |
| Compound 63 | 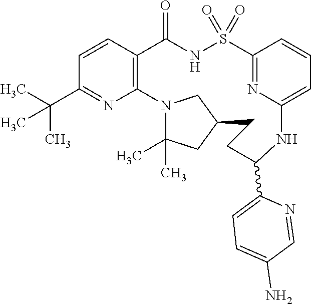 [SMILES: CC(C)(C)c1ccc2c(n1)N1C[C@@H](CCC(c3ccc(N)cn3)Nc3cccc(n3)S(=O)(=O)NC2=O)CC1(C)C] | 1.54 | 563.268 | 564.2 | LC method A |
| Compound 39 | 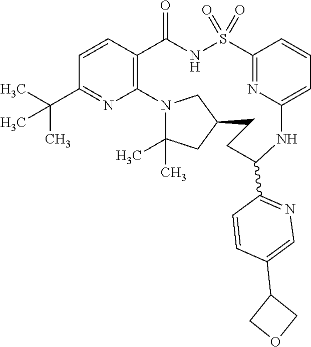 [SMILES: CC(C)(C)c1ccc2c(n1)N1C[C@@H](CCC(c3ccc(C4COC4)cn3)Nc3cccc(n3)S(=O)(=O)NC2=O)CC1(C)C] | 1.61 | 604.283 | 605.6 | LC method A |
| Compound 21 | 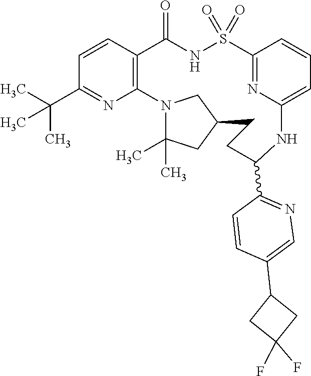 [SMILES: CC(C)(C)c1ccc2c(n1)N1C[C@@H](CCC(c3ccc(C4CC(F)(F)C4)cn3)Nc3cccc(n3)S(=O)(=O)NC2=O)CC1(C)C] | 1.79 | 638.285 | 639.3 | LC method A |

| Compound | | |
|---|---|---|
| Number | Structure | NMR |
| Compound 169 | 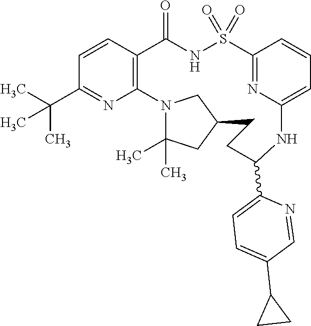 [SMILES: CC(C)(C)c1ccc2c(n1)N1C[C@@H](CCC(c3ccc(C4CC4)cn3)Nc3cccc(n3)S(=O)(=O)NC2=O)CC1(C)C] | ¹H NMR (400 MHz, DMSO-d₆) δ 12.43 (s, 1H), 8.58 (d, J = 2.0 Hz, 1H), 8.15-7.79 (m, 3H), 7.71 (t, J = 7.9 Hz, 1H), 7.46 (d, J = 7.9 Hz, 1H), 7.32 (d, J = 7.3 Hz, 1H), 6.92 (d, J = 8.4 Hz, 1H), 6.63 (d, J = 7.9 Hz, 1H), 5.17-4.95 (m, 1H), 3.20 (s, 1H), 2.77 (s, 1H), 2.29 (s, 1H), 2.18-2.02 (m, 2H), 1.85 (dd, J = 11.8, 5.6 Hz, 1H), 1.75 (d, J = 9.7 Hz, 1H), 1.55 (d, J = 7.2 Hz, 7H), 1.33-1.19 (m, 10H), 1.13 (d, J = 11.4 Hz, 1H), 1.09 (dt, J = 9.1, 3.2 Hz, 2H), 0.90-0.83 (m, 2H). |
| Compound 155 | 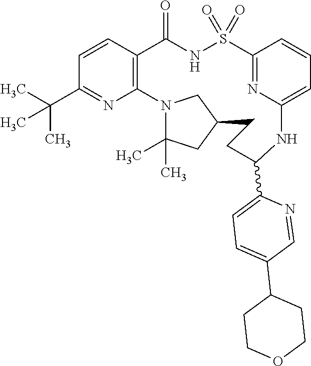 [SMILES: CC(C)(C)c1ccc2c(n1)N1C[C@@H](CCC(c3ccc(C4CCOCC4)cn3)Nc3cccc(n3)S(=O)(=O)NC2=O)CC1(C)C] | ¹H NMR (400 MHz, DMSO-d₆) δ 12.45 (s, 1H), 8.69 (s, 1H), 8.31 (s, 1H), 7.93 (d, J = 9.2 Hz, 2H), 7.72 (dd, J = 8.5, 7.2 Hz, 1H), 7.64 (d, J = 8.0 Hz, 1H), 7.19 (d, J = 7.2 Hz, 1H), 6.96 (d, J = 8.4 Hz, 1H), 6.66 (d, J = 8.0 Hz, 1H), 5.44 (s, 1H), 3.96 (dt, J = 11.1, 3.1 Hz, 2H), 3.42 (ddd, J = 11.2, 7.6, 5.1 Hz, 2H), 3.30 (t, J = 8.5 Hz, 1H), 3.08-2.96 (m, 1H), 2.69 (t, J = 10.2 Hz, 1H), 2.36 (s, 1H), 1.99 (s, 2H), 1.84 (dd, J = 11.6, 5.0 Hz, 1H), 1.74 (ddt, J = 12.1, 8.8, 3.4 Hz, 5H), 1.67 (s, 3H), 1.57 (t, J = 12.4 Hz, 1H), 1.51 (s, 3H), 1.49-1.42 (m, 1H), 1.28 (s, 9H). |
| Compound 153, hydrochloride salt | 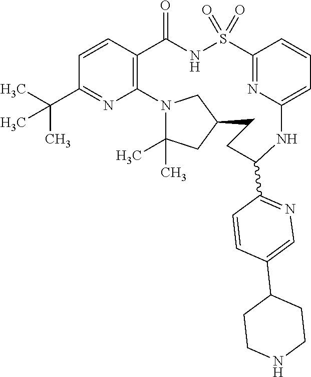 [SMILES: CC(C)(C)c1ccc2c(n1)N1C[C@@H](CCC(c3ccc(C4CCNCC4)cn3)Nc3cccc(n3)S(=O)(=O)NC2=O)CC1(C)C] | ¹H NMR (400 MHz, DMSO-d₆) δ 12.46 (s, 1H), 9.09 (s, 1H), 9.01 (s, 1H), 8.60 (d, J = 2.2 Hz, 1H), 8.20 (s, 1H), 8.04 (s, 2H), 7.71 (t, J = 7.9 Hz, 1H), 7.45 (d, J = 7.9 Hz, 1H), 7.32 (d, J = 7.3 Hz, 1H), 6.94 (d, J = 8.5 Hz, 1H), 6.62 (d, J = 7.9 Hz, 1H), 5.10 (s, 1H), 3.37 (d, J = 12.4 Hz, 2H), 3.21 (s, 1H), 3.06 (d, J = 11.9 Hz, 1H), 3.00 (d, J = 11.9 Hz, 1H), 2.94 (d, J = 11.9 Hz, 1H), 2.75 (s, 1H), 2.30 (s, 1H), 2.12 (s, 1H), 2.00 (d, J = 14.1 Hz, 2H), 1.96-1.81 (m, 3H), 1.76 (s, 1H), 1.55 (d, J = 5.8 Hz, 6H), 1.49 (d, J = 11.4 Hz, 1H), 1.27 (s, 9H), 1.15 (s, 1H). |
| Compound 135 | 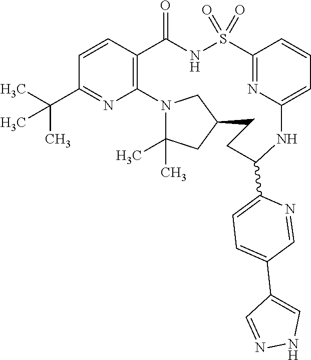 [SMILES: CC(C)(C)c1ccc2c(n1)N1C[C@@H](CCC(c3ccc(-c4cn[nH]c4)cn3)Nc3cccc(n3)S(=O)(=O)NC2=O)CC1(C)C] | ¹H NMR (400 MHz, DMSO-d₆) δ 12.42 (s, 1H), 8.96 (d, J = 2.2 Hz, 1H), 8.45 (s, 1H), 8.32 (s, 2H), 7.96 (d, J = 31.0 Hz, 2H), 7.70 (t, J = 7.9 Hz, 1H), 7.46 (d, J = 7.9 Hz, 1H), 7.31 (d, J = 7.2 Hz, 1H), 6.93 (d, J = 8.5 Hz, 1H), 6.63 (d, J = 7.9 Hz, 1H), 5.04 (s, 1H), 3.22 (s, 1H), 2.83 (s, 1H), 2.54 (s, 1H), 2.41-2.16 (m, 2H), 2.11 (dd, J = 15.0, 8.3 Hz, 1H), 1.86 (dd, J = 11.9, 5.7 Hz, 1H), 1.81-1.71 (m, 1H), 1.57 (s, 3H), 1.54 (s, 3H), 1.49 (d, J = 11.6 Hz, 1H), 1.27 (s, 9H), 1.18 (dd, J = 14.2, 8.6 Hz, 1H). |
| Compound 129 | 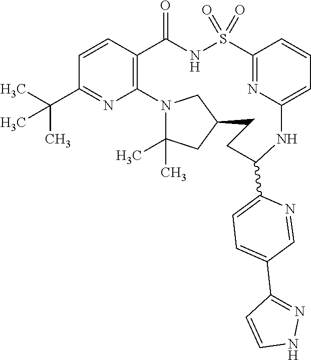 [SMILES: CC(C)(C)c1ccc2c(n1)N1C[C@@H](CCC(c3ccc(-c4cc[nH]n4)cn3)Nc3cccc(n3)S(=O)(=O)NC2=O)CC1(C)C] | ¹H NMR (400 MHz, DMSO-d₆) δ 12.38 (s, 1H), 9.04 (d, J = 2.2 Hz, 1H), 8.52-8.32 (m, 1H), 7.98-7.80 (m, 3H), 7.68 (t, J = 7.8 Hz, 1H), 7.52-7.42 (m, 1H), 7.28 (d, J = 7.3 Hz, 1H), 6.95-6.89 (m, 2H), 6.63 (d, J = 7.9 Hz, 1H), 5.03 (d, J = 7.2 Hz, 1H), 3.19 (s, 1H), 2.92 (s, 1H), 2.30 (d, J = 4.0 Hz, 1H), 2.17 (dd, J = 12.9, 7.9 Hz, 1H), 1.86 (dd, J = 11.8, 5.7 Hz, 1H), 1.82- 1.71 (m, 1H), 1.56 (d, J = 18.0 Hz, 6H), 1.49 (s, 1H), 1.49-1.31 (m, 1H), 1.29 (d, J = 5.9 Hz, 1H), 1.27 (s, 9H), 1.24-1.10 (m, 1H). |
| Compound 111 | 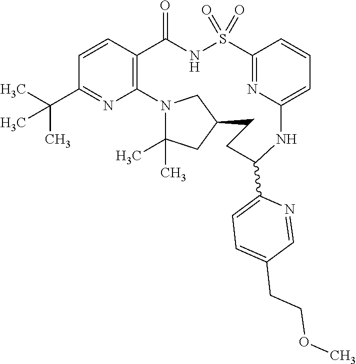 [SMILES: COCCc1ccc(C2CC[C@@H]3CN(c4nc(C(C)(C)C)ccc4C(=O)NS(=O)(=O)c4cccc(n4)N2)C(C)(C)C3)nc1] | ¹H NMR (400 MHz, DMSO-d₆) δ 12.41 (s, 1H), 8.59 (s, 1H), 8.13 (s, 1H), 7.91 (s, 2H), 7.70 (t, J = 7.9 Hz, 1H), 7.46 (d, J = 7.9 Hz, 1H), 7.31 (d, J = 7.2 Hz, 1H), 6.92 (d, J = 8.5 Hz, 1H), 6.63 (d, J = 7.9 Hz, 1H), 5.02 (s, 1H), 3.57 (d, J = 6.3 Hz, 2H), 3.23 (s, 3H), 3.21-3.14 (m, 1H), 2.94 (t, J = 6.3 Hz, 2H), 2.89-2.69 (m, 1H), 2.30 (s, 1H), 2.09 (d, J = 16.1 Hz, 1H), 1.85 (dd, J = 11.8, 5.7 Hz, 1H), 1.75 (dd, J = 15.3, 4.6 Hz, 1H), 1.55 (d, J = 10.6 Hz, 7H), 1.49 (d, J = 11.0 Hz, 1H), 1.27 (s, 9H), 1.23-1.13 (m, 1H). |
| Compound 102, hydrochloride salt | 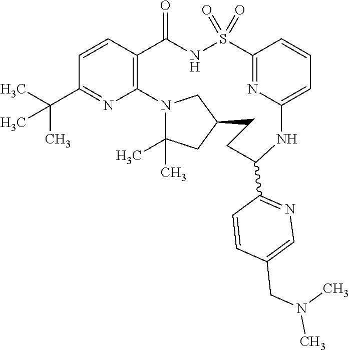 [SMILES: CN(C)Cc1ccc(C2CC[C@@H]3CN(c4nc(C(C)(C)C)ccc4C(=O)NS(=O)(=O)c4cccc(n4)N2)C(C)(C)C3)nc1] | ¹H NMR (400 MHz, DMSO-d₆) δ 12.51 (s, 1H), 11.09 (s, 1H), 8.77 (d, J = 2.2 Hz, 1H), 8.14 (d, J = 8.0 Hz, 1H), 7.84 (d, J = 9.0 Hz, 1H), 7.72-7.64 (m, 2H), 7.62 (d, J = 8.0 Hz, 1H), 7.13 (d, J = 7.2 Hz, 1H), 6.92 (d, J = 8.5 Hz, 1H), 6.66 (d, J = 8.0 Hz, 1H), 5.35 (q, J = 8.5 Hz, 1H), 4.31 (d, J = 5.8 Hz, 2H), 3.24 (t, J = 8.5 Hz, 1H), 2.76 (t, J = 10.3 Hz, 1H), 2.68 (dd, J = 13.5, 4.7 Hz, 6H), 2.27 (s, 1H), 1.95 (d, J = 9.2 Hz, 2H), 1.85 (dd, J = 11.7, 5.1 Hz, 1H), 1.80-1.72 (m, 1H), 1.65 (s, 3H), 1.58 (t, J = 12.3 Hz, 1H), 1.51 (s, 3H), 1.28 (s, 9H). |
| Compound 97 | 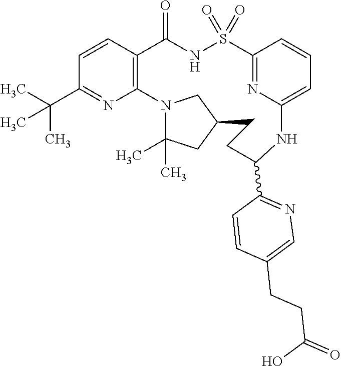 [SMILES: CC(C)(C)c1ccc2c(n1)N1C[C@@H](CCC(c3ccc(CCC(=O)O)cn3)Nc3cccc(n3)S(=O)(=O)NC2=O)CC1(C)C] | ¹H NMR (400 MHz, DMSO-d₆) δ 12.48 (s, 1H), 8.71 (s, 1H), 8.35 (s, 1H), 7.95 (s, 2H), 7.74 (t, J = 7.9 Hz, 1H), 7.63 (d, J = 8.0 Hz, 1H), 7.20 (d, J = 7.2 Hz, 1H), 6.97 (d, J = 8.5 Hz, 1H), 6.65 (d, J = 8.0 Hz, 1H), 5.46 (s, 1H), 3.30 (t, J = 8.2 Hz, 1H), 2.95 (t, J = 7.3 Hz, 2H), 2.67 (td, J = 9.3, 8.1, 5.3 Hz, 3H), 2.41 (s, 1H), 2.01 (s, 2H), 1.81 (ddd, J = 27.0, 13.6, 5.0 Hz, 2H), 1.67 (s, 3H), 1.56 (t, J = 12.3 Hz, 1H), 1.51 (s, 3H), 1.49-1.41 (m, 1H), 1.28 (s, 9H). CO₂H (s, 1H) not observed |
| Compound 90 | 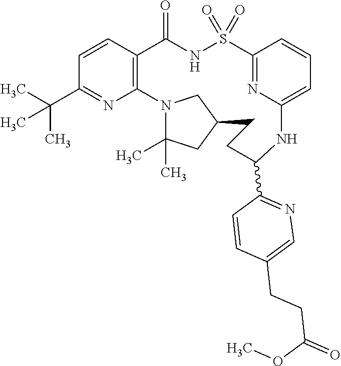 [SMILES: COC(=O)CCc1ccc(C2CC[C@@H]3CN(c4nc(C(C)(C)C)ccc4C(=O)NS(=O)(=O)c4cccc(n4)N2)C(C)(C)C3)nc1] | ¹H NMR (400 MHz, DMSO-d₆) δ 12.46 (s, 1H), 8.57 (s, 1H), 8.27-7.83 (m, 1H), 7.83- 7.74 (m, 1H), 7.73-7.56 (m, 3H), 7.15 (d, J = 7.2 Hz, 1H), 6.93 (dd, J = 8.6, 4.4 Hz, 1H), 6.65 (d, J = 8.0 Hz, 1H), 5.35 (dd, J = 10.9, 6.4 Hz, 1H), 3.57 (s, 3H), 3.24 (t, J = 8.7 Hz, 1H), 2.91 (t, J = 7.3 Hz, 2H), 2.72 (q, J = 7.9, 7.5 Hz, 3H), 2.36-2.30 (m, 1H), 2.01-1.91 (m, 2H), 1.84 (dd, J = 11.9, 5.2 Hz, 1H), 1.79-1.72 (m, 1H), 1.66 (s, 3H), 1.57 (t, J = 12.2 Hz, 1H), 1.51 (s, 3H), 1.45 (d, J = 13.3 Hz, 1H), 1.28 (s, 9H). |
| Compound 88 | 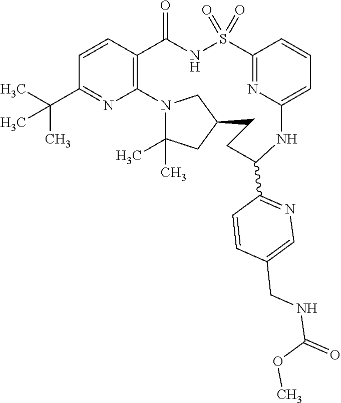 [SMILES: COC(=O)NCc1ccc(C2CC[C@@H]3CN(c4nc(C(C)(C)C)ccc4C(=O)NS(=O)(=O)c4cccc(n4)N2)C(C)(C)C3)nc1] | ¹H NMR (400 MHz, DMSO-d₆) δ 12.44 (s, 1H), 8.60 (s, 1H), 8.08 (s, 1H), 7.87 (d, J = 8.5 Hz, 1H), 7.81 (s, 2H), 7.71 (t, J = 7.9 Hz, 1H), 7.63 (d, J = 8.0 Hz, 1H), 7.17 (d, J = 7.3 Hz, 1H), 6.94 (d, J = 8.5 Hz, 1H), 6.65 (d, J = 8.0 Hz, 1H), 5.44-5.35 (m, 1H), 4.26 (d, J = 5.9 Hz, 2H), 3.52 (s, 3H), 3.27 (t, J = 8.5 Hz, 1H), 2.71 (t, J = 10.4 Hz, 1H), 2.35 (d, J = 12.8 Hz, 1H), 2.04- 1.94 (m, 2H), 1.85 (dd, J = 11.7, 5.1 Hz, 1H), 1.80-1.73 (m, 1H), 1.66 (s, 3H), 1.57 (t, J = 12.4 Hz, 1H), 1.51 (s, 3H), 1.49- 1.43 (m, 1H), 1.28 (s, 9H). |
| Compound 71 | 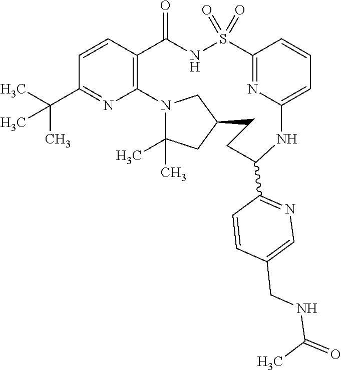 [SMILES: CC(=O)NCc1ccc(C2CC[C@@H]3CN(c4nc(C(C)(C)C)ccc4C(=O)NS(=O)(=O)c4cccc(n4)N2)C(C)(C)C3)nc1] | ¹H NMR (400 MHz, DMSO-d₆) 12.44 (s, 1H), 8.62 (s, 1H), 8.52 (s, 1H), 8.13 (s, 1H), 7.97-7.87 (m, 1H), 7.84 (s, 1H), 7.71 (t, J = 7.9 Hz, 1H), 7.63 (d, J = 8.0 Hz, 1H), 7.18 (d, J = 7.3 Hz, 1H), 6.95 (d, J = 8.5 Hz, 1H), 6.66 (d, J = 8.0 Hz, 1H), 5.41 (t, J = 9.9 Hz, 1H), 4.32 (d, J = 5.6 Hz, 2H), 3.28 (t, J = 8.3 Hz, 1H), 2.70 (t, J = 10.4 Hz, 1H), 2.45-2.33 (m, 1H), 2.00 (t, J = 9.3 Hz, 2H), 1.85 (s, 4H), 1.77 (d, J = 14.8 Hz, 1H), 1.67 (s, 3H), 1.57 (t, J = 12.3 Hz, 1H), 1.51 (s, 3H), 1.45 (d, J = 7.3 Hz, 1H), 1.28 (s, 9H). |
| Compound 39 | 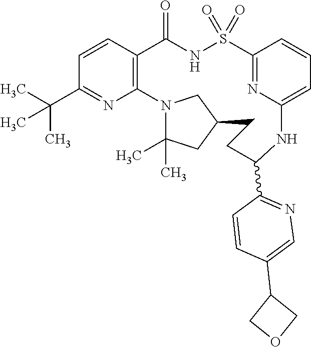 [SMILES: CC(C)(C)c1ccc2c(n1)N1C[C@@H](CCC(c3ccc(C4COC4)cn3)Nc3cccc(n3)S(=O)(=O)NC2=O)CC1(C)C] | ¹H NMR (400 MHz, Chloroform-d) δ 8.50 (d, J = 2.3 Hz, 1H), 7.94 (d, J = 8.1 Hz, 1H), 7.80 (dd, J = 8.0, 2.4 Hz, 1H), 7.61- 7.50 (m, 2H), 7.33 (d, J = 8.0 Hz, 1H), 6.86 (d, J = 8.1 Hz, 1H), 6.66 (dd, J = 8.0, 1.3 Hz, 1H), 5.86 (d, J = 8.4 Hz, 1H), 5.37 (m, 1H), 5.09 (ddd, J = 8.3, 6.2, 1.9 Hz, 2H), 4.72 (m, 2H), 4.25-4.19 (m, 1H), 3.53-3.40 (m, 2H), 3.09-2.95 (m, 2H), 2.20 (dd, J = 12.4, 8.0 Hz, 1H), 1.92 (m, 1H), 1.73 (m, 1H), 1.61-1.46 (m, 2H), 1.32 (s, 9H), 1.26 (t, J = 7.2 Hz, 6H) |

### Example 39: Preparation of (14S)-8-tert-Butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, Compound 128 (diastereomer 1) and (14S)-8-tert-butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, Compound 127 (diastereomer 2)

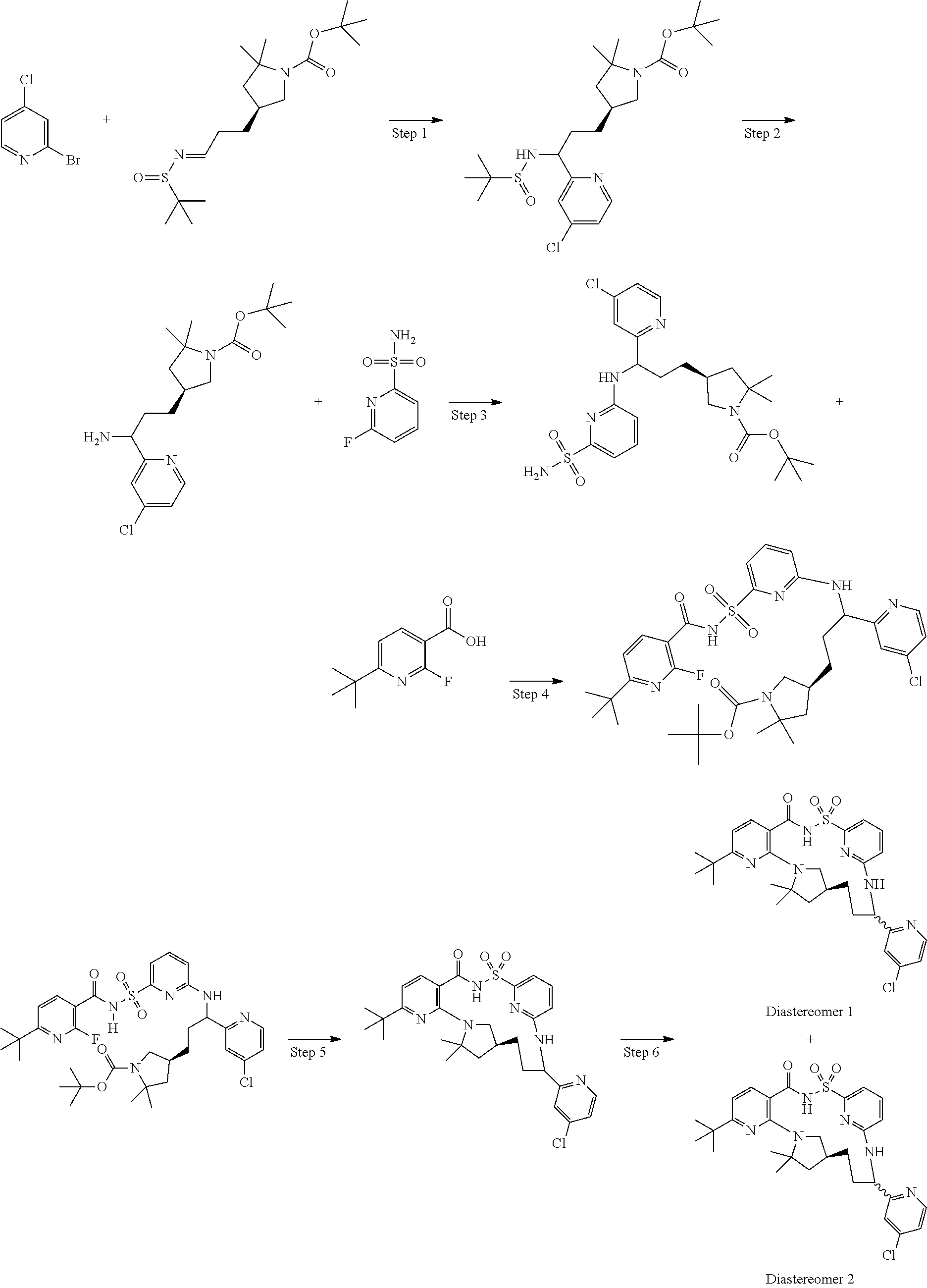

### Step 1: tert-Butyl (4S)-4-[3-(tert-butylsulfinylamino)-3-(4-chloro-2-pyridyl)propyl]-2,2-dimethyl-pyrrolidine-1-carboxylate

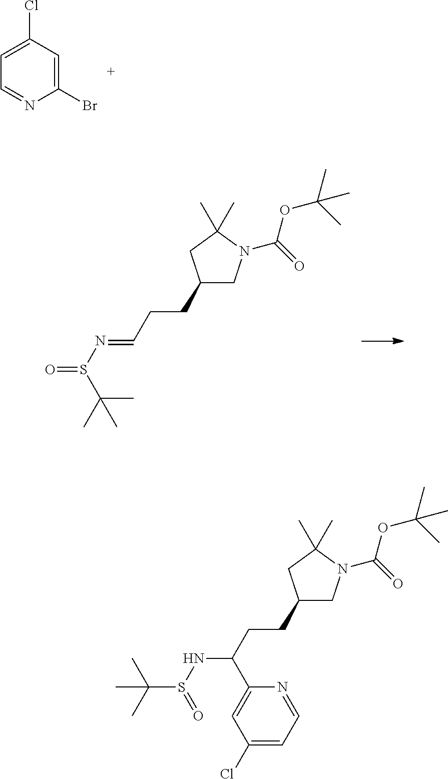

2-Bromo-4-chloro-pyridine (6.96 g, 35.444 mmol) was dissolved in Toluene (150 mL) and the solution was cooled in a dry ice acetone bath under nitrogen balloon. n-BuLi (14.5 mL of 2.5 M in hexanes, 36.250 mmol) was then added quick dropwise. The mixture was allowed to stir below −70° C. for 45 min. tert-butyl (4S)-4-[(3E)-3-tert-butylsulfinyliminopropyl]-2,2-dimethyl-pyrrolidine-1-carboxylate (6.45 g, 17.090 mmol) in THF (5 mL plus 2 mL rinse) was added dropwise quickly. The reaction was allowed to continue for 15 min and NH₄Cl (30 mL, saturated aqueous) was added, followed by EtOAc (150 mL) and water (200 mL). The mixture was allowed to warm up to rt. The layers were separated and the organic layer was washed with brine, dried over anhydrous MgSO₄, filtered and concentrated. The residue was purified by silica gel chromatography (120 g column), using 5-60% EtOAc in hexanes to afford tert-butyl (4S)-4-[3-(tert-butylsulfinylamino)-3-(4-chloro-2-pyridyl)propyl]-2,2-dimethyl-pyrrolidine-1-carboxylate as a pale yellow foam (8 g, 94%). ESI-MS m/z calc. 471.2322, found 472.7 (M+1)⁺; Retention time: 3.57 minutes (LC method B).

### Step 2: tert-Butyl (4S)-4-[3-amino-3-(4-chloro-2-pyridyl)propyl]-2,2-dimethyl-pyrrolidine-1-carboxylate

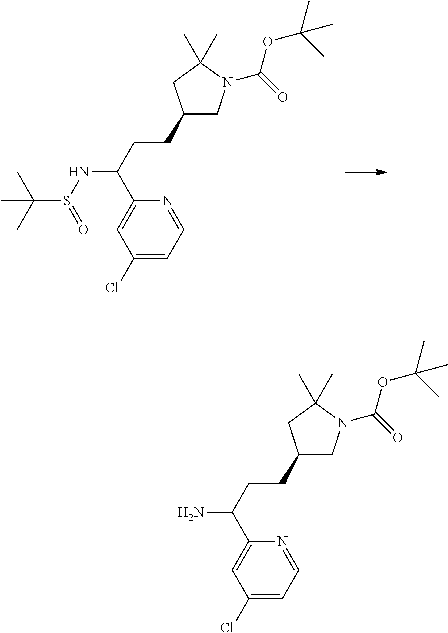 [SMILES: CC(C)(C)OC(=O)N1C[C@@H](CCC(NS(=O)C(C)(C)C)c2cc(Cl)ccn2)CC1(C)C ; CC(C)(C)OC(=O)N1C[C@@H](CCC(N)c2cc(Cl)ccn2)CC1(C)C]

tert-Butyl (4S)-4-[3-(tert-butylsulfinylamino)-3-(4-chloro-2-pyridyl)propyl]-2,2-dimethyl-pyrrolidine-1-carboxylate (8 g, 16.099 mmol) was dissolved in a solvent mixture of THF (50 mL) and Water (10 mL). Molecular iodine (1.23 g, 4.8462 mmol) was added. The dark brown solution was heated in a 50° C. oil bath for 3 h. It was then cooled to rt, treated with Na₂S₂O₃ (5 g in 40 mL saturated aqueous sodium bicarbonate) and extracted with EtOAc (40 mL). The organic layer was washed with brine (30 mL), dried over anhydrous Na₂SO₄, filtered and concentrated. The residue was purified by silica gel chromatography (80 g column), using 0-10% MeOH in DCM to afford tert-butyl (4S)-4-[3-amino-3-(4-chloro-2-pyridyl)propyl]-2,2-dimethyl-pyrrolidine-1-carboxylate as a light brown foam (4.33 g, 69%). ESI-MS m/z calc. 367.2027, found 368.5 (M+1)⁺; Retention time: 2.89 minutes (LC method B).

### Step 3: tert-Butyl (4S)-4-[3-(4-chloro-2-pyridyl)-3-[(6-sulfamoyl-2-pyridyl)amino]propyl]-2,2-dimethyl-pyrrolidine-1-carboxylate

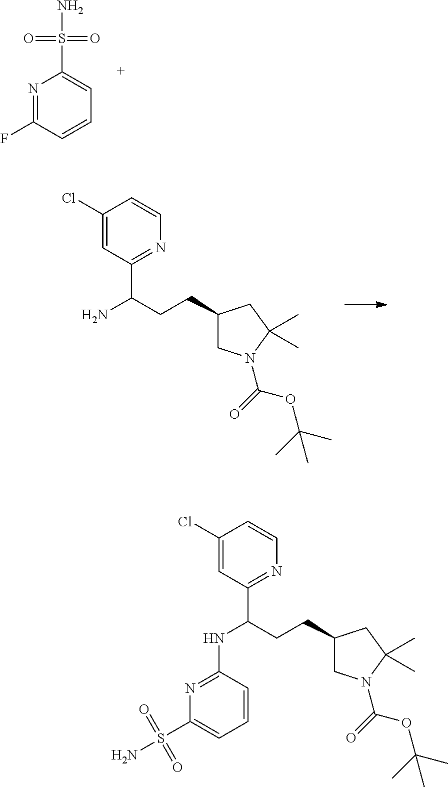 [SMILES: NS(=O)(=O)c1cccc(F)n1 ; CC(C)(C)OC(=O)N1C[C@@H](CCC(N)c2cc(Cl)ccn2)CC1(C)C ; CC(C)(C)OC(=O)N1C[C@@H](CCC(Nc2cccc(S(N)(=O)=O)n2)c2cc(Cl)ccn2)CC1(C)C]

To a mixture of tert-butyl (4S)-4-[3-amino-3-(4-chloro-2-pyridyl)propyl]-2,2-dimethyl-pyrrolidine-1-carboxylate (4.12 g, 10.638 mmol) and 6-fluoropyridine-2-sulfonamide (3.5 g, 18.874 mmol) in DMSO (10 mL) was added DIEA (6 mL, 34.447 mmol). The mixture was stirred at 110° C. for 27 h. The mixture was then cooled to rt and partitioned between EtOAc (200 mL) and saturated sodium bicarbonate (400 mL). The aqueous layer was extracted with more EtOAc (200 mL). The combined EtOAc solution was washed with brine (2×300 mL), dried over anhydrous sodium sulfate, filtered and concentrated. The residue was purified by silica gel chromatography, using 0-100% EtOAc in hexanes to afford tert-butyl (4S)-4-[3-(4-chloro-2-pyridyl)-3-[(6-sulfamoyl-2-pyridyl)amino]propyl]-2,2-dimethyl-pyrrolidine-1-carboxylate (4.08 g, 70%) as a beige powder. ¹H NMR (500 MHz, DMSO-d₆) δ 8.51 (d, J=5.3 Hz, 1H), 7.68-7.61 (m, 1H), 7.55-7.52 (m, 1H), 7.39 (dd, J=5.3, 2.1 Hz, 1H), 7.08 (s, 2H), 6.98 (d, J=7.4 Hz, 1H), 6.72 (d, J=8.4 Hz, 1H), 5.21 (bs, 1H), 3.60-3.46 (m, 1H), 2.83-2.69 (m, 1H), 2.17-2.01 (m, 1H), 1.94-1.76 (m, 3H), 1.42-1.28 (m, 16H), 1.23 (s, 3H). ESI-MS m/z calc. 523.202, found 524.5 (M+1)⁺; Retention time: 2.27 minutes (LC method H).

### Step 4: tert-Butyl (4S)-4-[3-[[6-[(6-tert-butyl-2-fluoro-pyridine-3-carbonyl)sulfamoyl]-2-pyridyl]amino]-3-(4-chloro-2-pyridyl)propyl]-2,2-dimethyl-pyrrolidine-1-carboxylate

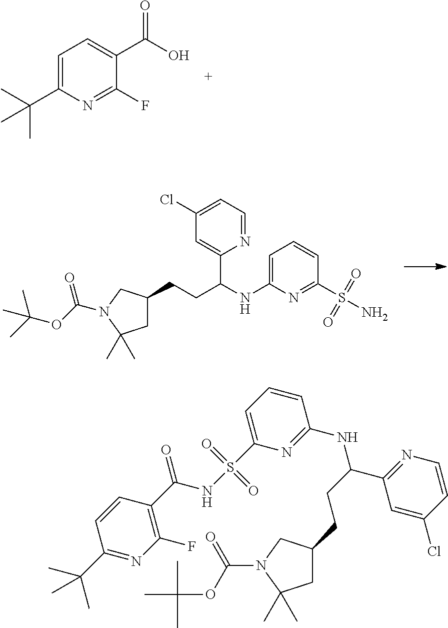 [SMILES: CC(C)(C)c1ccc(C(=O)O)c(F)n1 ; CC(C)(C)OC(=O)N1C[C@@H](CCC(Nc2cccc(S(N)(=O)=O)n2)c2cc(Cl)ccn2)CC1(C)C ; CC(C)(C)OC(=O)N1C[C@@H](CCC(Nc2cccc(S(=O)(=O)NC(=O)c3ccc(C(C)(C)C)nc3F)n2)c2cc(Cl)ccn2)CC1(C)C]

To a solution of 6-tert-butyl-2-fluoro-pyridine-3-carboxylic acid (1.84 g, 9.330 mmol) in THF (10 mL) was added CDI (1.56 g, 9.621 mmol) and the mixture was stirred at rt for 16 h then tert-butyl (4S)-4-[3-(4-chloro-2-pyridyl)-3-[(6-sulfamoyl-2-pyridyl)amino]propyl]-2,2-dimethyl-pyrrolidine-1-carboxylate (4 g, 7.632 mmol) was added followed by DBU (3 mL, 20.06 mmol) and the resulting mixture was stirred for 2 h at room temperature. The reaction was quenched with 1:1 mixture of saturated ammonium chloride and brine solutions, then extracted with ethyl acetate. The combined organic layers were washed with ‘brine, dried over sodium sulfate, filtered and evaporated. The resultant residue was purified by a reverse phase chromatography using a dual gradient run from 20-100% mobile phase B over 20.0 minutes (mobile phase A=H₂O (5 mM HCl), mobile phase B=CH₃CN) to give tert-butyl (4S)-4-[3-[[6-[(6-tert-butyl-2-fluoro-pyridine-3-carbonyl)sulfamoyl]-2-pyridyl]amino]-3-(4-chloro-2-pyridyl)propyl]-2,2-dimethyl-pyrrolidine-1-carboxylate as a mixture of diastereomers (3.24 g, 60%). ESI-MS m/z calc. 702.2767, found 703.3 (M+1)⁺; Retention time: 3.4 minutes (LC method R).

### Step 5: (14S)-8-tert-Butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione

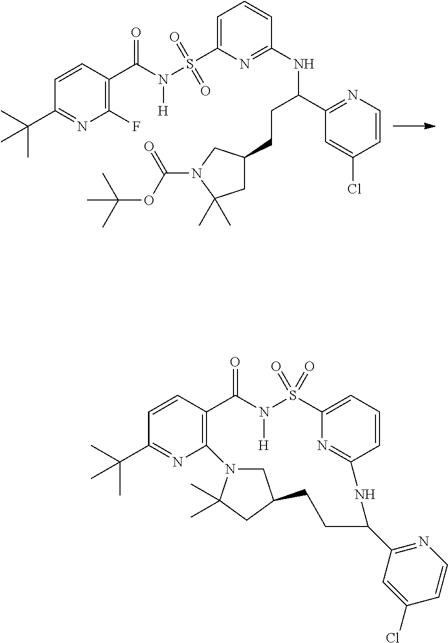 [SMILES: CC(C)(C)OC(=O)N1C[C@@H](CCC(Nc2cccc(S(=O)(=O)NC(=O)c3ccc(C(C)(C)C)nc3F)n2)c2cc(Cl)ccn2)CC1(C)C ; CC(C)(C)c1ccc2c(n1)N1C[C@@H](CCC(c3cc(Cl)ccn3)Nc3cccc(n3)S(=O)(=O)NC2=O)CC1(C)C]

To a solution of tert-butyl (4S)-4-[3-[[6-[(6-tert-butyl-2-fluoro-pyridine-3-carbonyl)sulfamoyl]-2-pyridyl]amino]-3-(4-chloro-2-pyridyl)propyl]-2,2-dimethyl-pyrrolidine-1-carboxylate (200 mg, 0.2844 mmol) in (1:4 TFA-DCM premixed solution) TFA (250 μL, 3.245 mmol) and DCM (750 μL) was stirred at rt and the solvent was removed. The residue was dissolved in NMP (2 mL) and potassium carbonate (454 mg, 3.285 mmol) was added. The mixture was heated at 140° C. for 16 h. The mixture was cooled to room temperature, diluted with 1N HCl and extracted with EtOAc (1×). The organic phase was washed with brine (1×), dried (sodium sulfate), filtered and concentrated to a brown oil, which was filtered and purified using a reverse phase HPLC-MS method using a dual gradient run from 30-99% mobile phase B over 15.0 minutes (mobile phase A=H₂O (5 mM HCl), mobile phase B=acetonitrile) giving (14S)-8-tert-butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (72 mg, 43%) as a mixture of diastereoisomers. ESI-MS m/z calc. 582.218, found 583.3 (M+1)⁺; Retention time: 0.62 minutes (LC method I).

### Step 6: (14S)-8-tert-Butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, Compound 128 (diastereomer 1) and (14S)-8-tert-butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, Compound 127 (diastereomer 2)

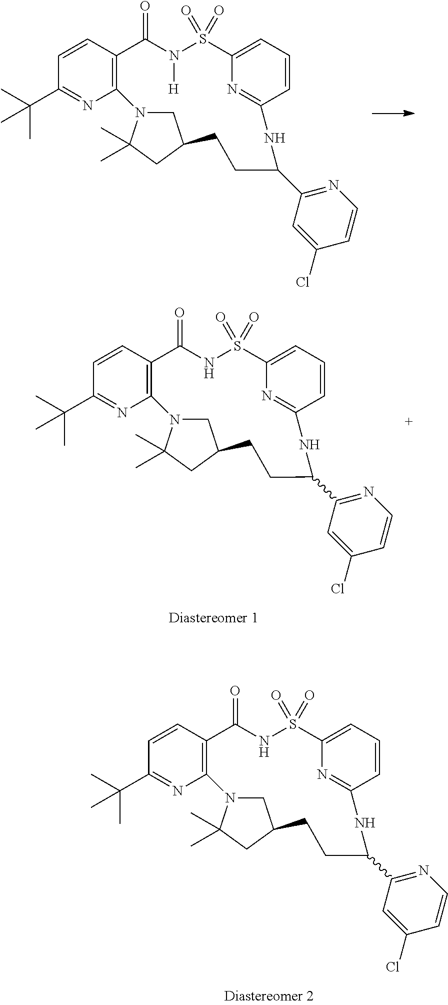 [SMILES: CC(C)(C)c1ccc2c(n1)N1C[C@@H](CCC(c3cc(Cl)ccn3)Nc3cccc(n3)S(=O)(=O)NC2=O)CC1(C)C ; CC(C)(C)c1ccc2c(n1)N1C[C@@H](CCC(c3cc(Cl)ccn3)Nc3cccc(n3)S(=O)(=O)NC2=O)CC1(C)C ; CC(C)(C)c1ccc2c(n1)N1C[C@@H](CCC(c3cc(Cl)ccn3)Nc3cccc(n3)S(=O)(=O)NC2=O)CC1(C)C]

The diastereomeric mixture of (14S)-8-tert-butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (30 mg, 0.05145 mmol) was separated by Chiral SFC with an OD-3 chiral column using 50-80% MeOH—NH₃ with 32 mL/minute flow rate giving two isomers as white solids:

First isomer to elute: Diastereomer 1 (PEAK-1): (14S)-8-tert-Butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (10.2 mg, 66%). ¹H NMR (400 MHz, DMSO-d₆) δ 12.48 (s, 1H), 8.50 (d, J 5.3 Hz, 1H), 7.62 (d, J=8.0 Hz, 3H), 7.54-7.50 (m, 1H), 7.40 (dt, J=5.3, 2.2 Hz, 1H), 7.10 (d, J 9.5 Hz, 1H), 6.88 (s, 1H), 6.65 (s, 1H), 5.27 (s, 1H), 3.19 (d, J 19.9 Hz, 1H), 2.75 (s, 1H), 2.22 (s, 1H), 1.91 (s, 2H), 1.83 (d, J 10.1 Hz, 2H), 1.72 (d, J 13.6 Hz, 1H), 1.64 (s, 3H), 1.50 (s, 3H), 1.44 (d, J=9.6 Hz, 1H), 1.28 (s, 9H). ESI-MS m/z calc. 582.218, found 583.3 (M+1)⁺; Retention time: 1.49 minutes (LC method G).

Second isomer to elute: Diastereomer 2 (PEAK-2): (14S)-8-tert-Butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (8.0 mg, 52%). ¹H NMR (400 MHz, DMSO-d₆) δ 12.49 (s, 1H), 8.51 (d, J 5.3 Hz, 1H), 7.74-7.57 (m, 3H), 7.53 (d, J=2.0 Hz, 1H), 7.40 (dd, J=5.3, 2.0 Hz, 1H), 7.12 (d, J=7.2 Hz, 1H), 6.89 (d, J=8.5 Hz, 1H), 6.66 (d, J=8.0 Hz, 1H), 5.26 (q, J 8.6 Hz, 1H), 3.20 (t, J=8.6 Hz, 1H), 2.75 (t, J=10.4 Hz, 1H), 2.23 (s, 1H), 1.91 (t, J 5.0 Hz, 2H), 1.84 (dd, J=11.8, 5.2 Hz, 1H), 1.73 (d, J=14.9 Hz, 1H), 1.65 (s, 3H), 1.62-1.52 (m, 2H), 1.51 (s, 3H), 1.28 (s, 9H). ESI-MS m/z calc. 582.218, found 583.3 (M+1)⁺; Retention time: 1.54 minutes (LC method G).

### Example 40: Preparation of tert-butyl 2′-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-1,2,3,6-tetrahydro-[4,4′-bipyridine]-1-carboxylate, diastereomer 1, diastereomer 2

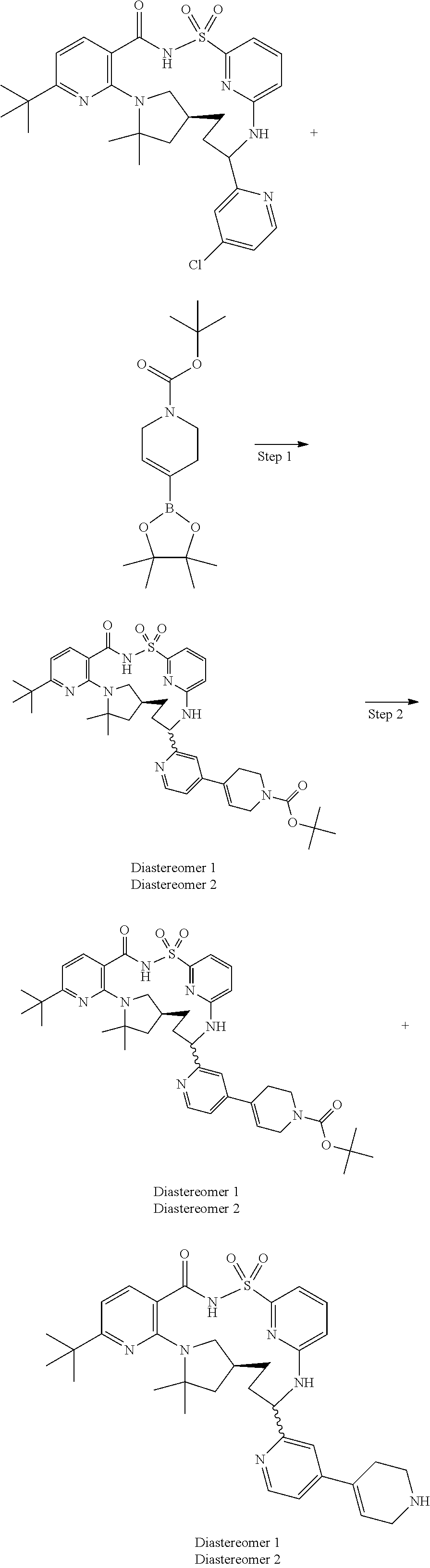

### Step 1: tert-Butyl 2′-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-1,2,3,6-tetrahydro-[4,4′-bipyridine]-1-carboxylate, diastereomer 1 and tert-butyl 2′-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-1,2,3,6-tetrahydro-[4,4′-bipyridine]-1-carboxylate, diastereomer 2

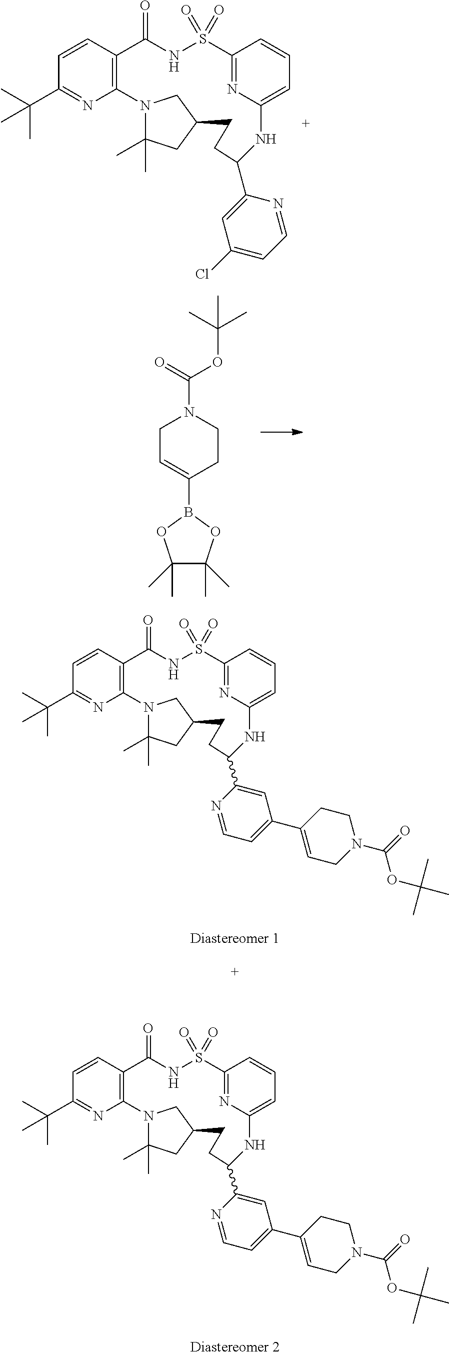

In a microwave vial (14S)-8-tert-butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (100 mg, 0.1715 mmol) and tert-butyl 4-(4,4,5,5-tetramethyl-1,3,2-dioxaborolan-2-yl)-3,6-dihydro-2H-pyridine-1-carboxylate (diastereomeric mixture, 84 mg, 0.2717 mmol) were combined in DMA (2 mL). Added to the mixture were [1,1′-bis(diphenylphosphino)ferrocene]dichloropalladium(II) (27 mg, 0.03690 mmol) and aqueous potassium carbonate (500 μL of 2 M, 1.000 mmol) and nitrogen was bubbled through the suspension for 1 minute. The reaction was capped and heated at 150° C. for 30 min in a microwave reactor. The reaction mixture was filtered through Whatman filter disc (puradisc 25 TF) and the filtrate was purified by a reverse phase HPLC-MS method using a dual gradient run of 50-99% mobile phase B over 15.0 minutes (mobile phase A=H₂O (5 mM HCl), mobile phase B=CH₃CN) to give two isomers as white solids.

First eluting isomer (PEAK-1), diastereomer 1: tert-Butyl 2′-[4(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-1,2,3,6-tetrahydro-[4,4′-bipyridine]-1-carboxylate (hydrochloride salt) (23.4 mg, 36%). ESI-MS m/z calc. 729.36725, found 730.5 (M+1)⁺; Retention time: 1.32 minutes (LC method G).

Second eluting isomer (PEAK-2), diastereomer 2: tert-butyl 2′-[(14S)-8-tert-butyl-12,12-dimethyl-2,2,4-tri oxo-2λ⁶-thia-3,9,11,18,23-pentaazatetracyclo[17.3.1.111,14.05,10]tetracosa-1(23),5(10),6,8,19,21-hexaen-17-yl]-1,2,3,6-tetrahydro-[4,4′-bipyridine]-1-carboxylate (hydrochloride salt) (38.6 mg, 59%). ESI-MS m/z calc. 729.36725, found 730.6 (M+1)⁺; Retention time: 1.38 minutes (LC method G).

### Step 2: tert-butyl 4-{2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-4-yl}piperidine-1-carboxylate, Compound 118, and (14S)-8-tert-butyl-12,12-dimethyl-17-[4-(piperidin-4-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione, Compound 124

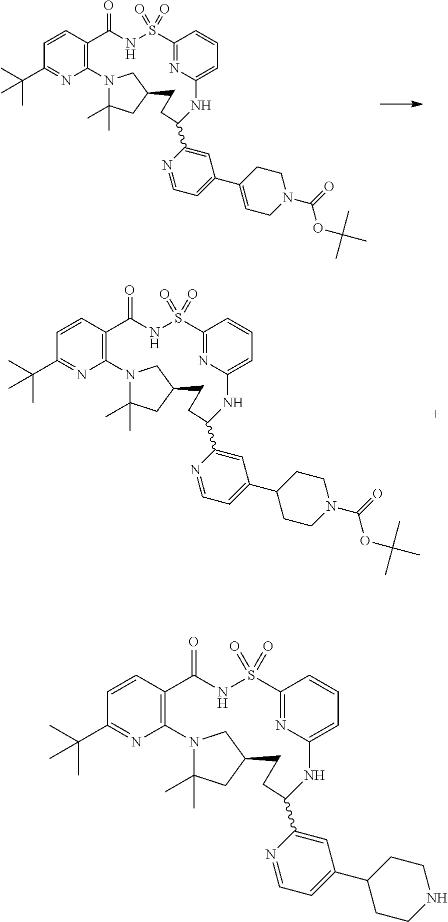

To a solution of tert-butyl 2′-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-1,2,3,6-tetrahydro-[4,4′-bipyridine]-1-carboxylate (hydrochloride salt), diastereomer 1 (23 mg, 0.03001 mmol, containing some Boc deprotected material) in EtOH (2 mL) was added Pd on C, 10%, wet, Degussa (10 mg, 0.0094 mmol) under nitrogen and the flask was evacuated and filled with hydrogen gas using a 3-way adaptor equipped with balloon. The reaction mixture was stirred under hydrogen for 4 h at rt and filtered through a small frit of celite using EtOAc as a solvent. The reaction mixture was filtered through Whatman filter disc (puradisc 25 TF) and filtrate was purified by a reverse phase HPLC-MS method using a dual gradient run of 30-99% mobile phase B over 15.0 minutes (mobile phase A=H₂O (5 mM HCl), mobile phase B=CH₃CN) to afford tert-butyl 4-{2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-4-yl}piperidine-1-carboxylate (hydrochloride salt) (4.8 mg, 42%). ¹H NMR (400 MHz, Chloroform-d) δ 9.35 (s, 1H), 8.54 (s, 1H), 8.45 (s, 1H), 7.86 (s, 1H), 7.69 (s, 1H), 7.59-7.50 (m, 2H), 7.39 (d, J=7.1 Hz, 1H), 6.87 (d, J=8.0 Hz, 1H), 6.75 (d, J=7.9 Hz, 1H), 5.66 (s, 1H), 4.29 (s, 2H), 3.46 (d, J=21.4 Hz, 1H), 3.28 (s, 1H), 3.03 (s, 1H), 2.85 (s, 2H), 2.39 (s, 2H), 1.95 (s, 3H), 1.85 (s, 2H), 1.63 (m, 5H), 1.57 (s, 3H), 1.45 (s, 9H), 1.30 (m, 11H). ESI-MS m/z calc. 731.3829, found 732.5 (M+1)⁺; Retention time: 1.28 minutes (LC method G); and (14S)-8-tert-butyl-12,12-dimethyl-17-[4-(piperidin-4-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione (dihydrochloride salt) (2.3 mg, 22%). ESI-MS m/z calc. 631.33044, found 632.5 (M+1)⁺; Retention time: 0.99 minutes (LC method G).

The compounds in the following tables were prepared in a manner analogous to that described above using tert-butyl 2′-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-1,2,3,6-tetrahydro-[4,4′-bipyridine]-1-carboxylate (hydrochloride salt), diastereomer 2 as a starting material:

| | | LCMS | | | |
|---|---|---|---|---|---|
| | | Retention | | | |
| Compound | | Time | Exact | | LCMS |
| Number | Structure | (min) | Mass | M + 1 | Method |
| Compound 117, hydrochloride salt | 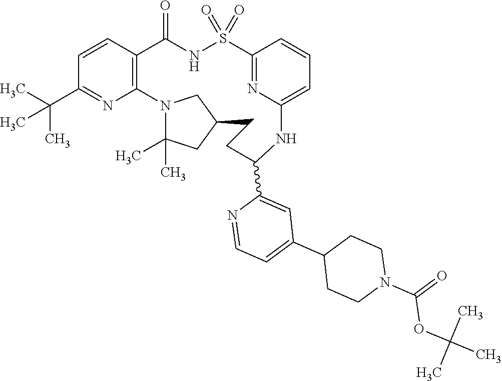 [SMILES: CC(C)(C)OC(=O)N1CCC(c2ccnc(C3CC[C@@H]4CN(c5nc(C(C)(C)C)ccc5C(=O)NS(=O)(=O)c5cccc(n5)N3)C(C)(C)C4)c2)CC1] | 1.35 | 731.383 | 732.5 | LC method G |
| Compound 123, dihydrochloride salt | 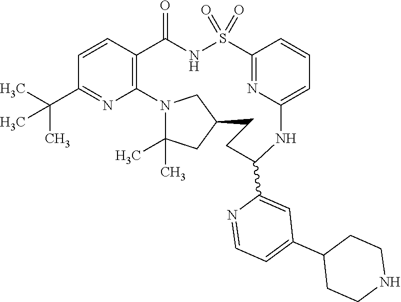 [SMILES: CC(C)(C)c1ccc2c(n1)N1C[C@@H](CCC(c3cc(C4CCNCC4)ccn3)Nc3cccc(n3)S(=O)(=O)NC2=O)CC1(C)C] | 1.16 | 631.33 | 632.3 | LC method G |

| Compound | | |
|---|---|---|
| Number | Structure | NMR |
| Compound 117, hydrochloride salt | 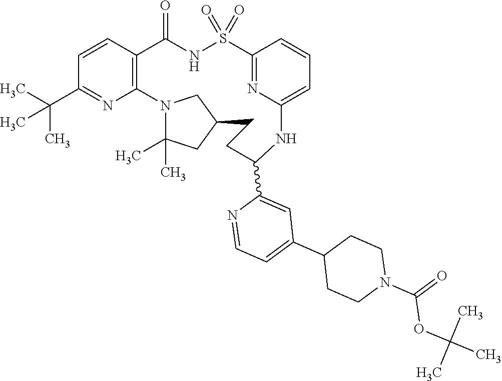 [SMILES: CC(C)(C)OC(=O)N1CCC(c2ccnc(C3CC[C@@H]4CN(c5nc(C(C)(C)C)ccc5C(=O)NS(=O)(=O)c5cccc(n5)N3)C(C)(C)C4)c2)CC1] | ¹H NMR (400 MHz, Chloroform-d) δ 10.45 (s, 1H), 8.52 (s, 1H), 7.80 (s, 2H), 7.52 (s, 2H), 7.39 (d, J = 7.0 Hz, 1H), 7.06 (s, 1H), 6.86 (s, 1H), 6.65 (d, J = 7.9 Hz, 1H), 5.82 (s, 1H), 4.26 (s, 2H), 3.46 (d, J = 22.6 Hz, 1H), 2.94 (s, 2H), 2.79 (s, 2H), 2.39 (s, 2H), 1.87 (d, J = 11.3 Hz, 3H), 1.75 (d, J = 13.3 Hz, 2H), 1.64 (m, 7H), 1.58 (s, 3H), 1.46 (s, 9H), 1.30 (s, 9H). |

### Example 41: Preparation of (14S)-17-[4-(3-aminopropyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, Compound 110 (diastereomer 1) and Compound 109 (diastereomer 2)

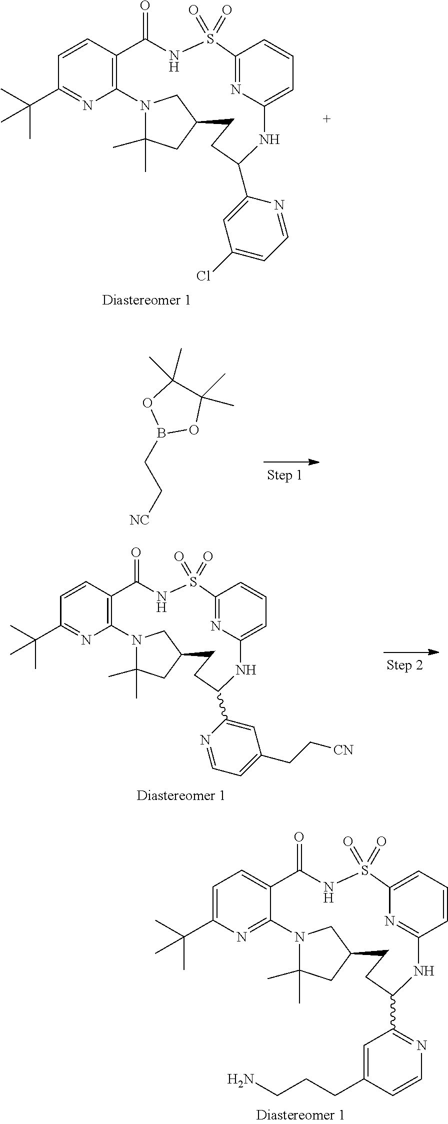

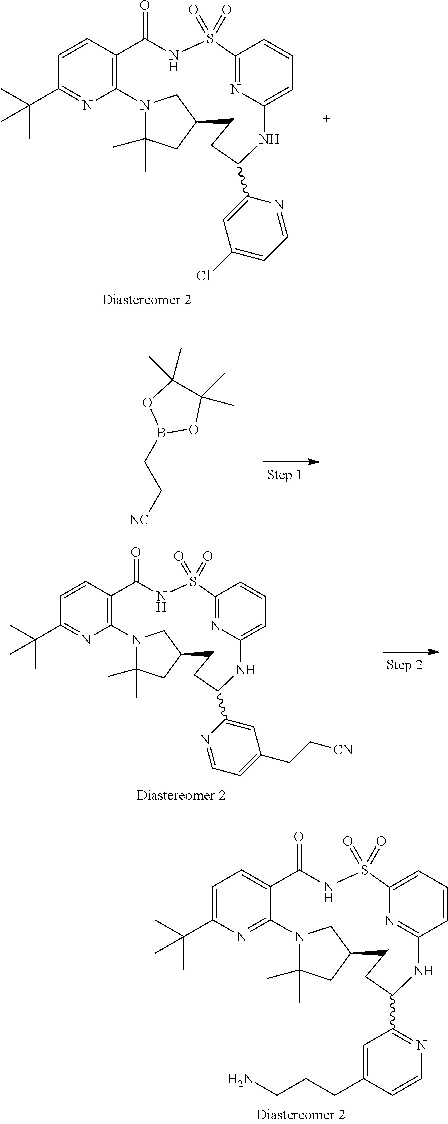

### Step 1: 3-{2-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}propanenitrile, diastereomer 1 and 3-{2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-1′7-yl]pyridin-4-yl}propanenitrile, diastereomer 2

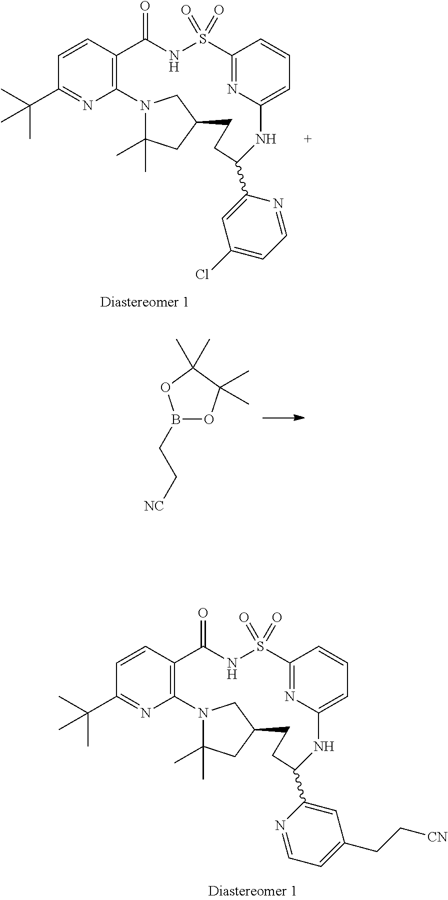 [SMILES: CC(C)(C)c1ccc2c(n1)N1C[C@@H](CCC(c3cc(Cl)ccn3)Nc3cccc(n3)S(=O)(=O)NC2=O)CC1(C)C ; CC1(C)OB(CCC#N)OC1(C)C ; CC(C)(C)c1ccc2c(n1)N1C[C@@H](CCC(c3cc(CCC#N)ccn3)Nc3cccc(n3)S(=O)(=O)NC2=O)CC1(C)C]

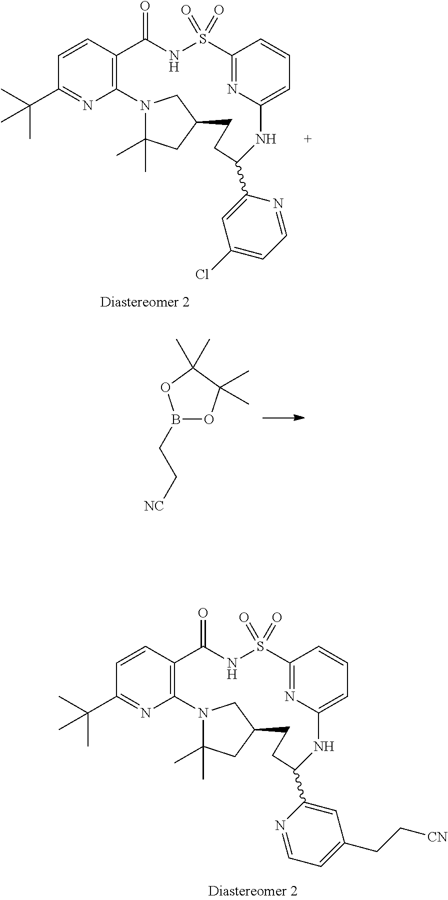 [SMILES: CC(C)(C)c1ccc2c(n1)N1C[C@@H](CCC(c3cc(Cl)ccn3)Nc3cccc(n3)S(=O)(=O)NC2=O)CC1(C)C ; CC1(C)OB(CCC#N)OC1(C)C ; CC(C)(C)c1ccc2c(n1)N1C[C@@H](CCC(c3cc(CCC#N)ccn3)Nc3cccc(n3)S(=O)(=O)NC2=O)CC1(C)C]

First Reaction:

In a microwave vial, (14S)-8-tert-butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 128 (diastereomer 1), 50 mg, 0.0857 mmol) and 3-(4,4,5,5-tetramethyl-1,3,2-dioxaborolan-2-yl)propanenitrile (26 mg, 0.1436 mmol) were combined in DMA (2 mL). Added to the mixture were [1,1′-bis(diphenylphosphino)ferrocene]dichloropalladium(II) (12 mg, 0.01640 mmol) and aqueous potassium carbonate (220 μL of 2 M, 0.4400 mmol) and nitrogen was bubbled through the suspension for 2 min. The reaction was capped and heated at 150° C. for 1 hour in a microwave reactor. The reaction mixture was filtered through Whatman filter disc (puradisc 25 TF) and the filtrate was purified by a reverse phase HPLC-MS method using a dual gradient run of 30-99% mobile phase B over 15.0 minutes (mobile phase A=H₂O (5 mM HCl), mobile phase B=CH₃CN) to afford 3-{2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}propanenitrile (diastereomer 1, 10.9 mg, 21%). ESI-MS m/z calc. 601.2835, found 602.1 (M+1)⁺; Retention time: 0.37 minutes (LC method I).

Second Reaction:

In a microwave vial, (14S)-8-tert-butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 127 (diastereomer 2), 50 mg, 0.08574 mmol) and 3-(4,4,5,5-tetramethyl-1,3,2-dioxaborolan-2-yl)propanenitrile (26 mg, 0.1436 mmol) were combined in DMA (2 mL). Added to the mixture were [1,1′-bis(diphenylphosphino)ferrocene]dichloropalladium(II) (12 mg, 0.0164 mmol) and aqueous potassium carbonate (220 μL of 2 M, 0.440 mmol) and nitrogen was bubbled through the suspension for 2 minute. The reaction was capped and heated at 150° C. for 1 hour in a microwave oven. The reaction mixture was filtered through Whatman filter disc (puradisc 25 TF) and the filtrate was purified by a reverse phase HPLC-MS method using a dual gradient run of 30-99% mobile phase B over 15.0 minutes (mobile phase A=H₂O (5 mM HCl), mobile phase B=CH₃CN) to afford 3-{2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}propanenitrile (diastereomer 2, 8.9 mg, 17%). ESI-MS m/z calc. 601.2835, found 602.1 (M+1)⁺; Retention time: 0.43 minutes (LC method I).

### Step 2: (14S)-17-[4-(3-Aminopropyl)pyridin-2-yl]-8-tert-butyl-12,12-dimethyl-R⁶-thia-3,9,11,18,23-pentaazatetracyclo[17.3.1.111,14.05,10]tetracosa-1(22),5,7,9,19(23),20-hexaene-2,2,4-trione, Compound 110 (diastereomer 1) and (14S)-17-[4-(3-aminopropyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, Compound 109 (diastereomer 2)

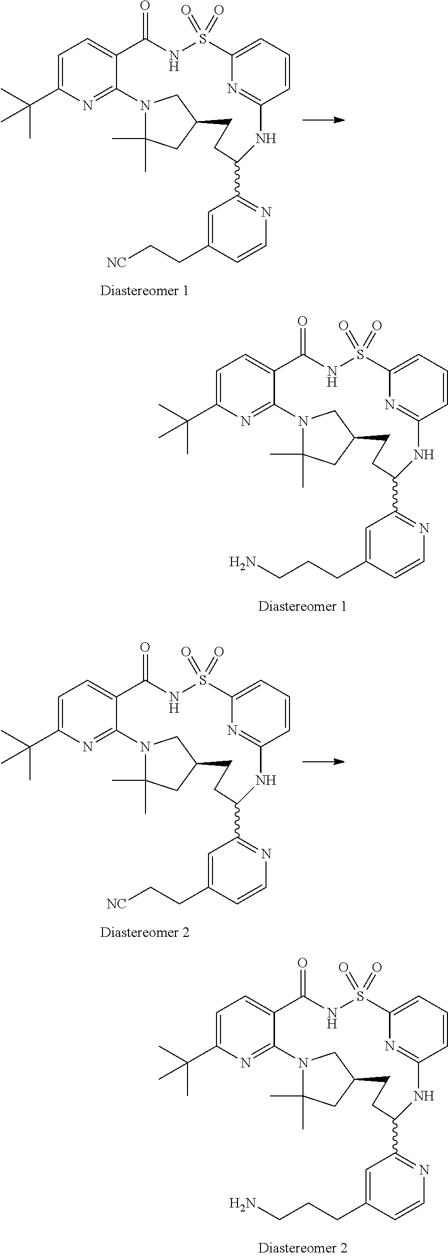 [SMILES: CC(C)(C)c1ccc2c(n1)N1C[C@@H](CCC(c3cc(CCC#N)ccn3)Nc3cccc(n3)S(=O)(=O)NC2=O)CC1(C)C ; CC(C)(C)c1ccc2c(n1)N1C[C@@H](CCC(c3cc(CCCN)ccn3)Nc3cccc(n3)S(=O)(=O)NC2=O)CC1(C)C ; CC(C)(C)c1ccc2c(n1)N1C[C@@H](CCC(c3cc(CCC#N)ccn3)Nc3cccc(n3)S(=O)(=O)NC2=O)CC1(C)C ; CC(C)(C)c1ccc2c(n1)N1C[C@@H](CCC(c3cc(CCCN)ccn3)Nc3cccc(n3)S(=O)(=O)NC2=O)CC1(C)C]

First Reaction:

To a solution of 3-{2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}propanenitrile (diastereomer 1, 8 mg, 0.01329 mmol) in MeOH (1 mL) was added NiCl₂.H₂O (8 mg, 0.05420 mmol) and the reaction was cooled in an ice bath. To the reaction vial, sodium borohydride (5 mg, 0.1322 mmol) was added slowly (vigorous reaction) and the reaction was allowed to stir from 0° C. to rt for about 2 h. The reaction mixture was filtered through a Whatman filter disc (puradisc 25 TF) and the filtrate was purified by a reverse phase HPLC-MS method using a dual gradient run of 20-80% mobile phase B over 15.0 minutes (mobile phase A=H₂O (5 mM HCl), mobile phase B=CH₃CN) to give (14S)-17-[4-(3-aminopropyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (dihydrochloride salt) (diastereomer 1, 2.7 mg, 29%) ESI-MS m/z calc. 605.3148, found 606.3 (M+1)⁺; Retention time: 1.28 minutes (LC method A).

Second Reaction:

To a solution of 3-{2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}propanenitrile (diastereomer 2, 8 mg, 0.01329 mmol) (PEAK-2) in MeOH (1 mL) was added NiCl₂.H₂O (8 mg, 0.05420 mmol) and the mixture was cooled in an ice bath. To the reaction vial, sodium borohydride (5 mg, 0.1322 mmol) was added slowly (vigorous reaction) and the reaction was allowed to stir from 0° C. to rt for about 2 h. The reaction mixture was filtered through a Whatman filter disc (puradisc 25 TF) and the filtrate was purified by a reverse phase HPLC-MS method using a dual gradient run of 20-80% mobile phase B over 15.0 minutes (mobile phase A=H₂O (5 mM HCl), mobile phase B=CH₃CN) to give (14S)-17-[4-(3-aminopropyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (dihydrochloride salt) (diastereomer 2, 2.0 mg, 22%). ESI-MS m/z calc. 605.3148, found 606.5 (M+1)⁺; Retention time: 1.39 minutes (LC method A).

The following is a list of boron reagents that are commercially available:
2-(3,6-Dihydro-2H-pyran-4-yl)-4,4,5,5-tetramethyl-1,3,2-dioxaborolane
(Dimethylamino)methyl-trifluoro-boranuide (potassium salt)
tert-Butyl N-[2-(4,4,5,5-tetramethyl-1,3,2-dioxaborolan-2-yl)ethyl]carbamate
1H-Pyrazol-3-ylboronic acid
4-(4,4,5,5-Tetramethyl-1,3,2-dioxaborolan-2-yl)-1H-pyrazole
(tert-Butylamino)methyl-trifluoro-boranuide

The compounds in the following tables were prepared in a manner analogous to that described above using boron reagents given in the table above. For each compound, diastereomer 1 was obtained when using (14S)-8-tert-butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 128 (diastereomer 1)) as a starting material. Diastereomer 2 was obtained when using Compound 127 (diastereomer 2) as a starting material.

| | | LCMS | | | |
|---|---|---|---|---|---|
| | | Retention | | | |
| Compound | Structure | Time | Exact | | LCMS |
| Number | | (min) | Mass | M + 1 | Method |
| Compound 126 (diastereomer 1), hydrochloride salt | 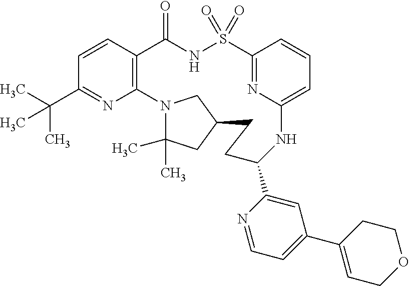 [SMILES: CC(C)(C)c1ccc2c(n1)N1C[C@@H](CC[C@@H](c3cc(C4=CCOCC4)ccn3)Nc3cccc(n3)S(=O)(=O)NC2=O)CC1(C)C] | 0.97 | 630.299 | 631.4 | LC method G |
| Compound 125 (diastereomer 2), hydrochloride salt | 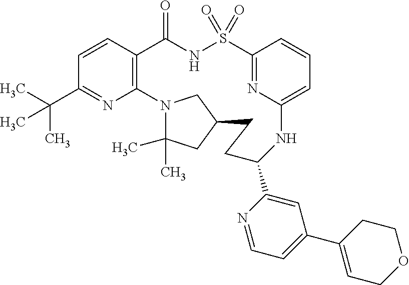 [SMILES: CC(C)(C)c1ccc2c(n1)N1C[C@@H](CC[C@@H](c3cc(C4=CCOCC4)ccn3)Nc3cccc(n3)S(=O)(=O)NC2=O)CC1(C)C] | 1.05 | 630.299 | 631.3 | LC method G |
| Compound 87 (diastereomer 1), dihydrochloride salt | 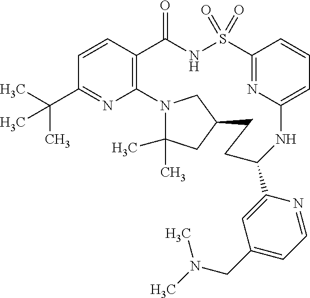 [SMILES: CN(C)Cc1ccnc([C@@H]2CC[C@@H]3CN(c4nc(C(C)(C)C)ccc4C(=O)NS(=O)(=O)c4cccc(n4)N2)C(C)(C)C3)c1] | 1.46 | 605.315 | 606.3 | LC method A |
| Compound 86 (diastereomer 2), dihydrochloride salt | 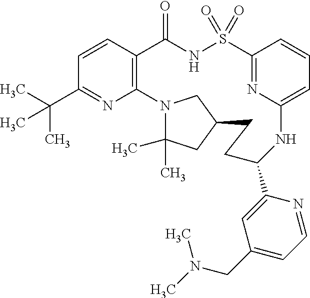 [SMILES: CN(C)Cc1ccnc([C@@H]2CC[C@@H]3CN(c4nc(C(C)(C)C)ccc4C(=O)NS(=O)(=O)c4cccc(n4)N2)C(C)(C)C3)c1] | 1.57 | 605.315 | 606.2 | LC method A |
| Compound 85 (diastereomer 1), dihydrochloride salt | 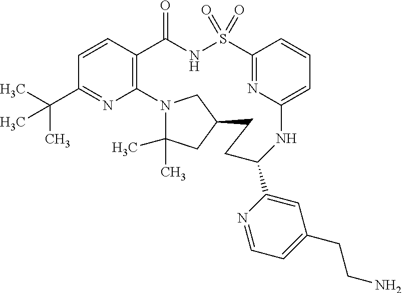 [SMILES: CC(C)(C)c1ccc2c(n1)N1C[C@@H](CC[C@@H](c3cc(CCN)ccn3)Nc3cccc(n3)S(=O)(=O)NC2=O)CC1(C)C] | 1.27 | 591.299 | 592.1 | LC method A |
| Compound 84 (diastereomer 2), dihydrochloride salt | 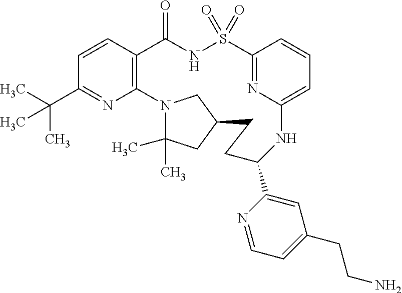 [SMILES: CC(C)(C)c1ccc2c(n1)N1C[C@@H](CC[C@@H](c3cc(CCN)ccn3)Nc3cccc(n3)S(=O)(=O)NC2=O)CC1(C)C] | 1.4 | 591.299 | 592.2 | LC method A |
| Compound 74 (diastereomer 1), dihydrochloride salt | 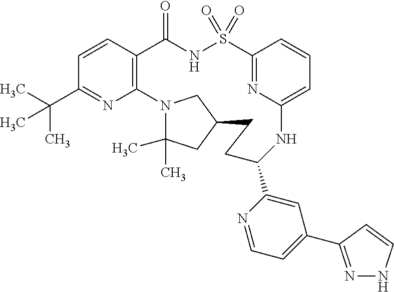 [SMILES: CC(C)(C)c1ccc2c(n1)N1C[C@@H](CC[C@@H](c3cc(-c4cc[nH]n4)ccn3)Nc3cccc(n3)S(=O)(=O)NC2=O)CC1(C)C] | 1.59 | 614.279 | 615.5 | LC method A |
| Compound 73 (diastereomer 2), dihydrochloride salt | 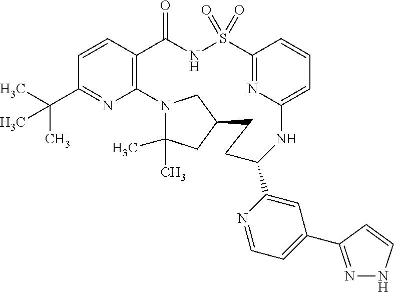 [SMILES: CC(C)(C)c1ccc2c(n1)N1C[C@@H](CC[C@@H](c3cc(-c4cc[nH]n4)ccn3)Nc3cccc(n3)S(=O)(=O)NC2=O)CC1(C)C] | 1.65 | 614.279 | 615.5 | LC method A |
| Compound 69 (diastereomer 1), dihydrochloride salt | 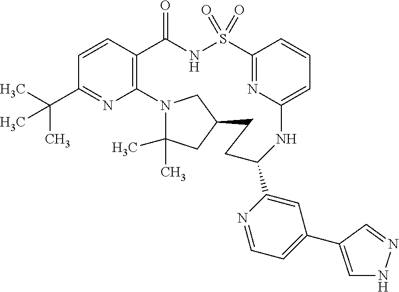 [SMILES: CC(C)(C)c1ccc2c(n1)N1C[C@@H](CC[C@@H](c3cc(-c4cn[nH]c4)ccn3)Nc3cccc(n3)S(=O)(=O)NC2=O)CC1(C)C] | 1.54 | 614.279 | 615.3 | LC method A |
| Compound 68 (diastereomer 2), dihydrochloride salt | 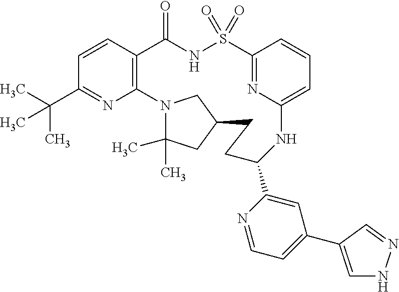 [SMILES: CC(C)(C)c1ccc2c(n1)N1C[C@@H](CC[C@@H](c3cc(-c4cn[nH]c4)ccn3)Nc3cccc(n3)S(=O)(=O)NC2=O)CC1(C)C] | 1.59 | 614.279 | 615.3 | LC method A |
| Compound 62 (diastereomer 1), hydrochloride salt | 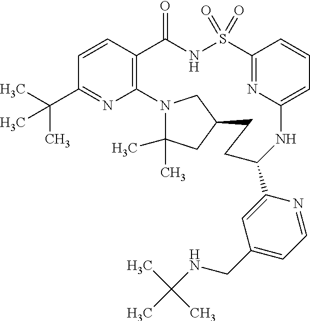 [SMILES: CC(C)(C)NCc1ccnc([C@@H]2CC[C@@H]3CN(c4nc(C(C)(C)C)ccc4C(=O)NS(=O)(=O)c4cccc(n4)N2)C(C)(C)C3)c1] | 1.49 | 633.346 | 634.5 | LC method A |
| Compound 61 (diastereomer 2), hydrochloride salt | 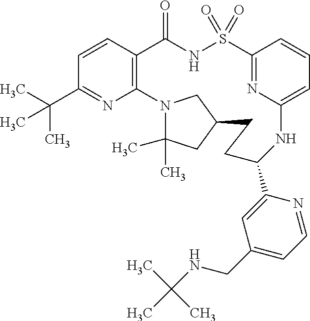 [SMILES: CC(C)(C)NCc1ccnc([C@@H]2CC[C@@H]3CN(c4nc(C(C)(C)C)ccc4C(=O)NS(=O)(=O)c4cccc(n4)N2)C(C)(C)C3)c1] | 1.59 | 633.346 | 634.5 | LC method A |
| Compound 122 (diastereomer 1), hydrochloride salt | 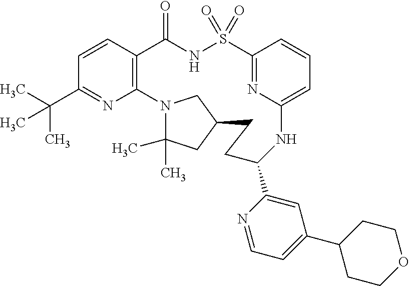 [SMILES: CC(C)(C)c1ccc2c(n1)N1C[C@@H](CC[C@@H](c3cc(C4CCOCC4)ccn3)Nc3cccc(n3)S(=O)(=O)NC2=O)CC1(C)C] | 0.91 | 632.314 | 633.3 | LC method G |
| Compound 121 (diastereomer 2), hydrochloride salt | 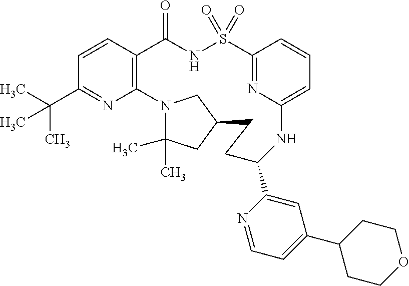 [SMILES: CC(C)(C)c1ccc2c(n1)N1C[C@@H](CC[C@@H](c3cc(C4CCOCC4)ccn3)Nc3cccc(n3)S(=O)(=O)NC2=O)CC1(C)C] | 0.99 | 632.314 | 633.5 | LC method G |

| Compound | | |
|---|---|---|
| Number | Structure | NMR |
| Compound 86 (diastereomer 2), dihydrochloride salt | 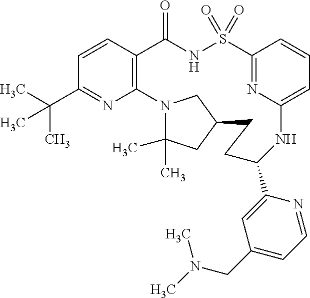 [SMILES: CN(C)Cc1ccnc([C@@H]2CC[C@@H]3CN(c4nc(C(C)(C)C)ccc4C(=O)NS(=O)(=O)c4cccc(n4)N2)C(C)(C)C3)c1] | ¹H NMR (400 MHz, DMSO-d₆) δ 12.54 (s, 1H), 11.36 (s, 1H), 8.76 (d, J = 5.3 Hz, 1H), 8.02 (s, 1H), 7.86 (d, J = 8.5 Hz, 1H), 7.77 (s, 1H), 7.69 (dd, J = 8.5, 7.2 Hz, 1H), 7.63 (d, J = 8.0 Hz, 1H), 7.15 (d, J = 7.2 Hz, 1H), 6.95 (s, 1H), 6.66 (d, J = 8.0 Hz, 1H), 5.45- 5.35 (m, 1H), 4.41 (s, 2H), 3.28 (t, J = 8.4 Hz, 1H), 2.75 (d, J = 10.1 Hz, 1H), 2.69 (d, J = 3.7 Hz, 6H), 2.33 (s, 1H), 2.04 (s, 2H), 1.85 (dd, J = 11.8, 5.2 Hz, 1H), 1.77 (d, J = 14.8 Hz, 1H), 1.65 (s, 3H), 1.59 (d, J = 12.5 Hz, 1H), 1.51 (s, 3H), 1.45 (d, J = 9.6 Hz, 1H), 1.28 (s, 9H). |
| Compound 122 (diastereomer 1), hydrochloride salt | 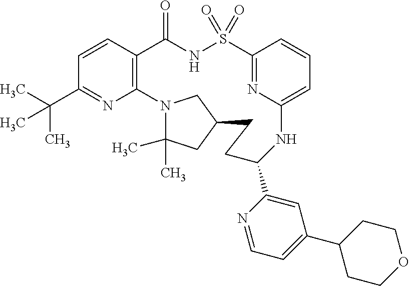 [SMILES: CC(C)(C)c1ccc2c(n1)N1C[C@@H](CC[C@@H](c3cc(C4CCOCC4)ccn3)Nc3cccc(n3)S(=O)(=O)NC2=O)CC1(C)C] | ¹H NMR (400 MHz, DMSO-d₆) δ 11.96 (s, 1H), 8.46 (s, 1H), 7.61 (d, J = 9.0 Hz, 1H), 7.47 (s, 2H), 7.33 (s, 1H), 7.19 (s, 1H), 7.03 (d, J = 36.4 Hz, 1H), 6.62 (s, 1H), 6.52 - 6.36 (m, 1H), 4.87 (s, 1H), 4.06 (dq, J = 4.9, 2.8 Hz, 1H), 3.93 (d, J = 11.2 Hz, 2H), 3.61 (q, J = 5.2 Hz, 1H), 3.42 (t, J = 11.5 Hz, 2H), 2.80 (d, J = 9.4 Hz, 1H), 2.05 (dq, J = 5.4, 2.6 Hz, 1H), 1.70 (s, 2H), 1.63 (s, 4H), 1.47 (s, 4H), 1.26 (s, 9H), 1.24 (s, 3H), 1.20 (s, 3H). |

### Example 42: Preparation of (14S)-17-(4-aminopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 1, Compound 49 and (14S)-17-(4-aminopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 2, Compound 48

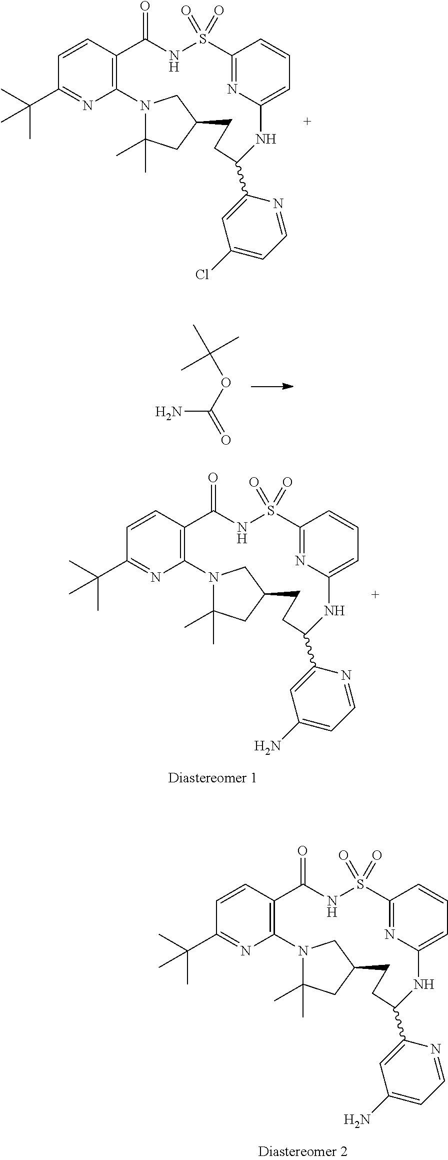

To a solution of (14S)-8-tert-butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (hydrochloride salt) (diastereomeric mixture, 25 mg, 0.04035 mmol) in DMF (2 mL) was added tert-butyl carbamate (9.6 mg, 0.08195 mmol), [2-(2-aminophenyl)phenyl]-methylsulfonyloxy-palladium; dicyclohexyl-[2-(2,4,6-triisopropylphenyl)phenyl]phosphane (8.4 mg, 0.0099 mmol) and cesium carbonate (43 mg, 0.1320 mmol). The mixture was degassed with nitrogen for 2 min and the vial was capped and heated at 150° C. for 30 min in a microwave oven. The reaction mixture was filtered through Whatman filter disc (puradisc 25 TF) and the filtrate was purified by reverse phase HPLC-MS using a Luna C₁₈ column (75×30 mm, 5 μm particle size) sold by Phenomenex (pn: 00C-4252-U0-AX), and a dual gradient run of 30-99% mobile phase B over 15.0 minutes (mobile phase A=H₂O (5 mM HCl), mobile phase B=CH₃CN). The solvent was removed and the resultant residue was dissolved in a 1:9 mixture of TFA (100 μL, 1.298 mmol) and DCM (400 mL) and the mixture was stirred at rt for 1 h. The solvent was removed and the residue was purified by a reverse phase HPLC-MS method using a dual gradient run of 1-99% mobile phase B over 15.0 minutes (mobile phase A=H₂O (5 mM HCl), mobile phase B=CH₃CN) to afford two isomers:

First eluting PEAK-1, diastereomer 1: (14S)-17-(4-Aminopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (3.1 mg, 26%). ESI-MS m/z calc. 563.2679, found 564.26 (M+1)⁺; Retention time: 1.48 minutes (LC method A).

Second eluting enantiomer, PEAK-2, diastereomer 2: (14S)-17-(4-aminopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (1.2 mg, 10%). ESI-MS m/z calc. 563.2679, found 564.26 (M+1)⁺; Retention time: 1.52 minutes (LC method A).

### Example 43: Preparation of 2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-4-carboxamide, diastereomer 1, Compound 83, and diastereomer 2, Compound 82 and 2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-4-carbonitrile, Compound 144 (diastereomer 1) and Compound 143 (diastereomer 2)

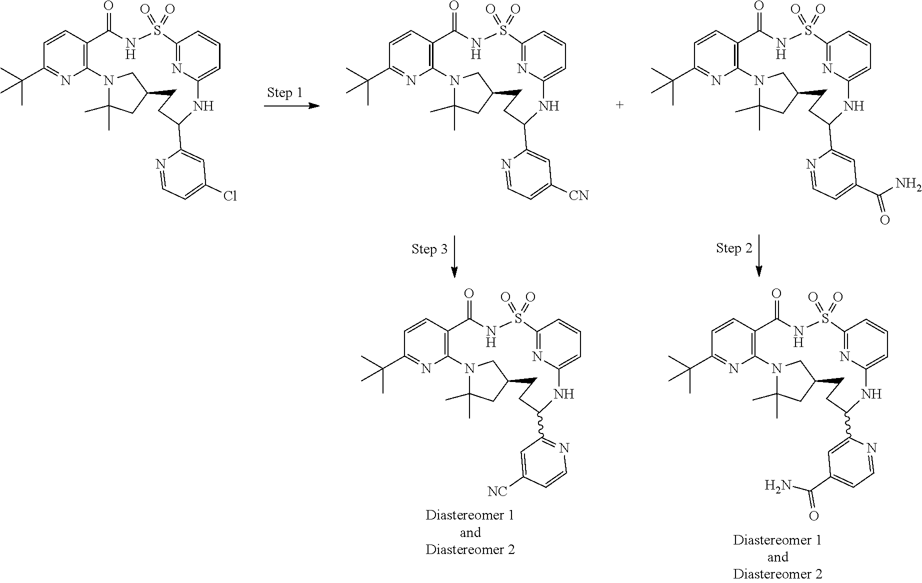 [SMILES: CC(C)(C)c1ccc2c(n1)N1C[C@@H](CCC(c3cc(Cl)ccn3)Nc3cccc(n3)S(=O)(=O)NC2=O)CC1(C)C ; CC(C)(C)c1ccc2c(n1)N1C[C@@H](CCC(c3cc(C#N)ccn3)Nc3cccc(n3)S(=O)(=O)NC2=O)CC1(C)C ; CC(C)(C)c1ccc2c(n1)N1C[C@@H](CCC(c3cc(C(N)=O)ccn3)Nc3cccc(n3)S(=O)(=O)NC2=O)CC1(C)C ; CC(C)(C)c1ccc2c(n1)N1C[C@@H](CCC(c3cc(C#N)ccn3)Nc3cccc(n3)S(=O)(=O)NC2=O)CC1(C)C ; CC(C)(C)c1ccc2c(n1)N1C[C@@H](CCC(c3cc(C(N)=O)ccn3)Nc3cccc(n3)S(=O)(=O)NC2=O)CC1(C)C]

### Step 1: 2-{8-tert-Butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl}pyridine-4-carbonitrile and 2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-4-carboxamide

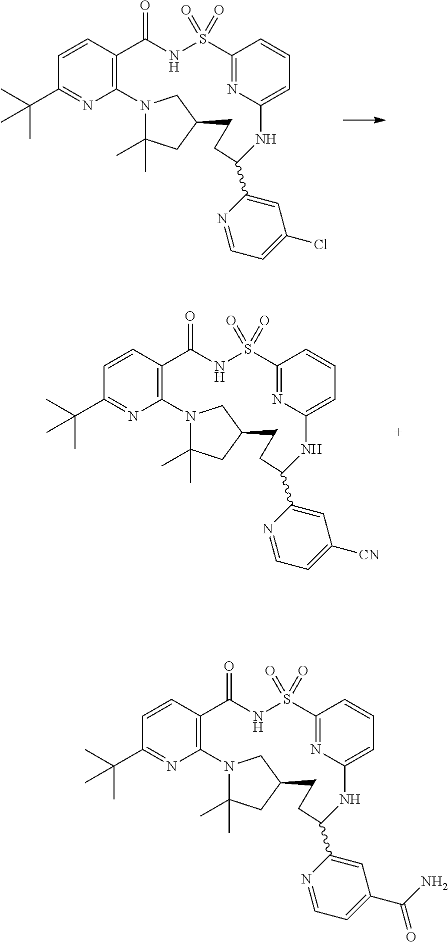 [SMILES: CC(C)(C)c1ccc2c(n1)N1C[C@@H](CCC(c3cc(Cl)ccn3)Nc3cccc(n3)S(=O)(=O)NC2=O)CC1(C)C ; CC(C)(C)c1ccc2c(n1)N1C[C@@H](CCC(c3cc(C#N)ccn3)Nc3cccc(n3)S(=O)(=O)NC2=O)CC1(C)C ; CC(C)(C)c1ccc2c(n1)N1C[C@@H](CCC(c3cc(C(N)=O)ccn3)Nc3cccc(n3)S(=O)(=O)NC2=O)CC1(C)C]

In a microwave vial (14S)-8-tert-butyl-17-(4-chloropyridin-2-yl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (diastereomeric mixture, 100 mg, 0.1715 mmol) was dissolved in DMF (2 mL) and Water (500 μL). To the mixture was added dicyanozinc (28 mg, 0.2384 mmol) followed by Tris(dibenzylideneacetone)dipalladium(0) (9 mg, 0.00983 mmol) and dicyclohexyl-[2-(2,6-dimethoxyphenyl)phenyl]phosphane (8 mg, 0.0195 mmol). The mixture was purged with nitrogen, capped and irradiated in the microwave for 60 minutes at 150° C. Cooled to room temperature. The reaction mixture was filtered through Whatman filter disc (puradisc 25 TF) and the filtrate was purified by a reverse phase HPLC-MS method using a dual gradient run of 30-99% mobile phase B over 15.0 minutes (mobile phase A=H₂O (5 mM HCl), mobile phase B=CH₃CN) to afford the major product 2-{8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl}pyridine-4-carbonitrile (47 mg, 48%). ESI-MS m/z calc. 573.2522, found 574.18 (M+1)⁺; Retention time: 1.55 minutes (LC method A).

A side product resulting from the hydrolysis of the CN was isolated: 2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-4-carboxamide (25.4 mg, 25%). ESI-MS m/z calc. 591.26276, found 592.2 (M+1)⁺; Retention time: 0.96 minutes (LC method A).

### Step 2: 2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-4-carboxamide, Compound 83 (diastereomer 1) and 2-[(14S)-8-tert-butyl-12,12-dimethyl-2,2,4-trioxo-2λ⁶-thia-3,9,11,18,23-pentaazatetracyclo [17.3.1.111,14.05,10]tetracosa-1(23),5(10),6,8,19,21-hexaen-17-yl]pyridine-4-carboxamide, Compound 82 (diastereomer 2)

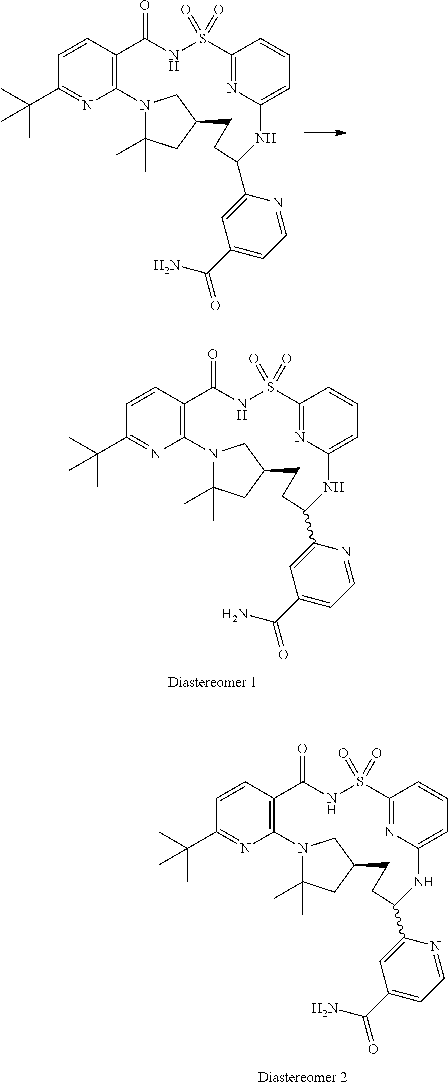 [SMILES: CC(C)(C)c1ccc2c(n1)N1C[C@@H](CCC(c3cc(C(N)=O)ccn3)Nc3cccc(n3)S(=O)(=O)NC2=O)CC1(C)C ; CC(C)(C)c1ccc2c(n1)N1C[C@@H](CCC(c3cc(C(N)=O)ccn3)Nc3cccc(n3)S(=O)(=O)NC2=O)CC1(C)C ; CC(C)(C)c1ccc2c(n1)N1C[C@@H](CCC(c3cc(C(N)=O)ccn3)Nc3cccc(n3)S(=O)(=O)NC2=O)CC1(C)C]

The diastereomeric mixture of 2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-4-carboxamide (25 mg, 0.04225 mmol) was separated by SFC using a Princeton 2-EP (250×10 mm), 5 nm; column using 20% MeOH (No modifier), 80% CO₂ with 10 mL/minute flow rate giving two isomers:

First eluting enantiomer, diastereomer 1: 2-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-4-carboxamide (hydrochloride salt) (3.4 mg, 26%). ESI-MS m/z calc. 591.26276, found 592.2 (M+1)⁺; Retention time: 1.64 minutes (LC method A).

Second eluting enantiomer, diastereomer 2: 2-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-4-carboxamide (hydrochloride salt) (8.6 mg, 65%). ¹H NMR (400 MHz, DMSO-d₆) δ 8.13 (d, J=7.0 Hz, 1H), 7.75 (s, 1H), 7.69 (t, J=7.8 Hz, 1H), 7.64 (d, J=8.1 Hz, 1H), 7.17 (d, J=7.3 Hz, 1H), 6.98-6.88 (m, 2H), 6.79 (s, 1H), 6.64 (d, J=7.9 Hz, 1H), 5.34-5.22 (m, 1H), 3.29 (d, J=8.5 Hz, 1H), 2.70 (t, J=10.1 Hz, 1H), 2.32 (s, 1H), 1.97-1.87 (m, 2H), 1.83 (dd, J=11.8, 5.2 Hz, 1H), 1.78-1.72 (m, 1H), 1.67 (s, 3H), 1.56 (d, J=12.4 Hz, 1H), 1.50 (s, 3H), 1.43 (dd, J=14.2, 10.9 Hz, 1H), 1.28 (s, 9H), 2 protons not visible. ESI-MS m/z calc. 591.26276, found 592.2 (M+1)⁺; Retention time: 1.66 minutes (LC method A).

### Step 3: 2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-4-carbonitrile, Compound 144 (diastereomer 1) and 2-[(14S)-8-tert-butyl-12,12-dimethyl-2,2,4-trioxo-2λ⁶-thia-3,9,11,18,23-pentaazatetracyclo [17.3.1.111,14.05,10]tetracosa-1(23),5(10),6,8,19,21-hexaen-17-yl]pyridine-4-carbonitrile, Compound 143 (diastereomer 2)

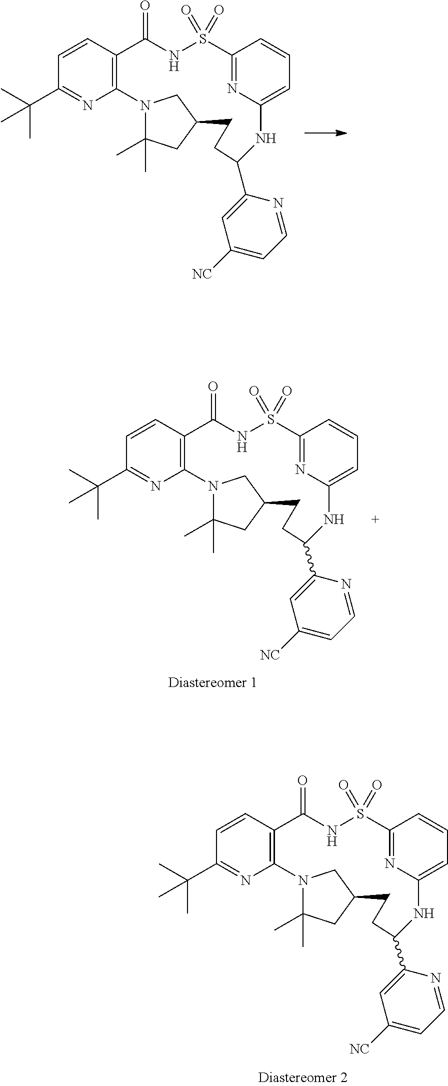 [SMILES: CC(C)(C)c1ccc2c(n1)N1C[C@@H](CCC(c3cc(C#N)ccn3)Nc3cccc(n3)S(=O)(=O)NC2=O)CC1(C)C ; CC(C)(C)c1ccc2c(n1)N1C[C@@H](CCC(c3cc(C#N)ccn3)Nc3cccc(n3)S(=O)(=O)NC2=O)CC1(C)C ; CC(C)(C)c1ccc2c(n1)N1C[C@@H](CCC(c3cc(C#N)ccn3)Nc3cccc(n3)S(=O)(=O)NC2=O)CC1(C)C]

The diastereomeric mixture of 2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-4-carbonitrile (18 mg, 0.03137 mmol) was separated by Chiral SFC with OD-3 chiral column using 50-80% MeOH—NH₃ with 32 mL/minute flow rate giving two isomers:

First eluting enantiomer, diastereomer 1: 2-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-4-carbonitrile (7.4 mg, 82%). ¹H NMR (400 MHz, DMSO-d₆) δ 12.48 (s, 1H), 8.79 (d, J=5.0 Hz, 1H), 7.85 (s, 1H), 7.73 (dd, J=5.0, 1.5 Hz, 2H), 7.64 (d, J=7.9 Hz, 2H), 7.11 (s, 1H), 6.87 (s, 1H), 6.65 (s, 1H), 5.34 (s, 1H), 3.24 (s, 1H), 2.78 (s, 1H), 2.23 (s, 1H), 1.91-1.81 (m, 2H), 1.74 (d, J=13.9 Hz, 1H), 1.64 (s, 3H), 1.51 (s, 6H), 1.28 (s, 9H). ESI-MS m/z calc. 573.2522, found 574.3 (M+1)⁺; Retention time: 1.63 minutes (LC method G).

Second eluting enantiomer, diastereomer 2: 2-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridine-4-carbonitrile (8.2 mg, 91%). ¹H NMR (400 MHz, DMSO-d₆) δ 12.36 (s, 1H), 8.80 (d, J=5.1 Hz, 1H), 8.03 (s, 1H), 7.75 (d, J=5.0 Hz, 1H), 7.65 (s, 1H), 7.47 (s, 1H), 7.25 (s, 1H), 7.12 (s, 1H), 6.85 (s, 1H), 6.62 (s, 1H), 4.87 (s, 1H), 3.51-3.35 (m, 1H), 3.23-3.11 (m, 1H), 3.05-2.88 (m, 1H), 2.39-2.25 (m, 1H), 2.13 (s, 1H), 1.83 (s, 1H), 1.74 (s, 1H), 1.60 (s, 3H), 1.52 (s, 3H), 1.38 (s, 1H), 1.27 (s, 9H), 1.14 (d, J=30.1 Hz, 1H). ESI-MS m/z calc. 573.2522, found 574.2 (M+1)⁺; Retention time: 1.61 minutes (LC method G).

### Example 44: Preparation of tert-butyl N-({2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}methyl)carbamate, Compound 120 (diastereomer 1) and Compound 119 (diastereomer 2)

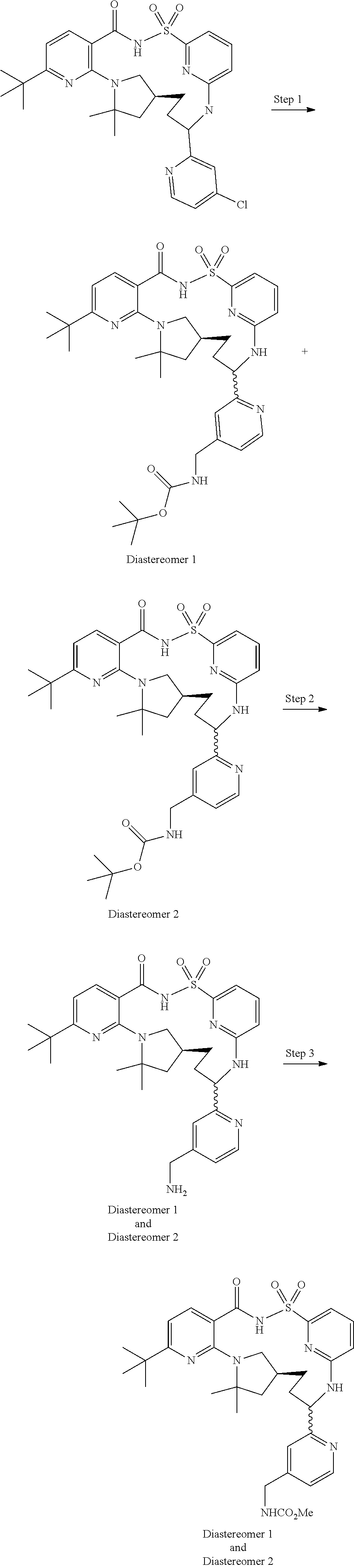

### Step 1: tert-butyl N-({2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}methyl)carbamate, Compound 120 (diastereomer 1) and tert-butyl N-({2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}methyl)carbamate, Compound 119 (diastereomer 2)

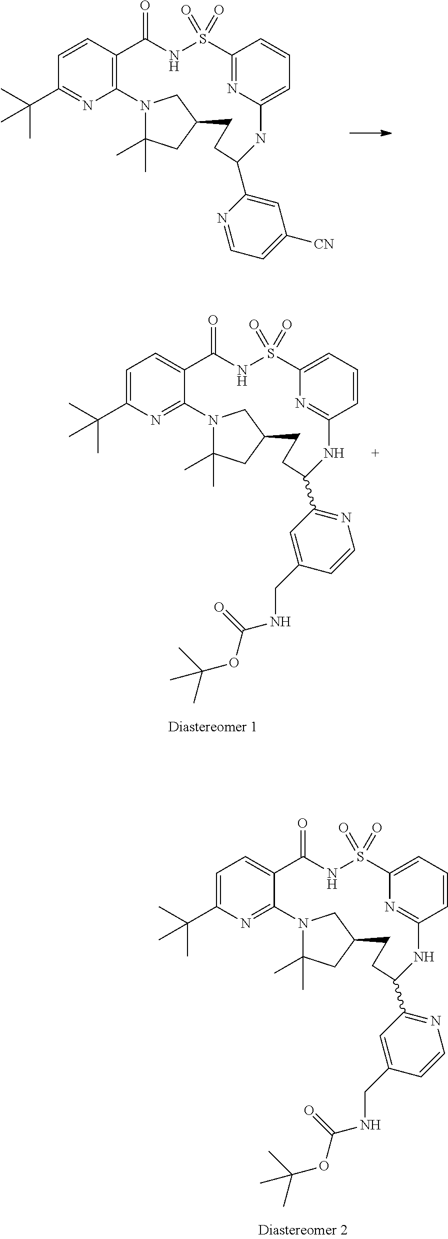 [SMILES: CC(C)(C)c1ccc2c(n1)N1C[C@@H](CCC(c3cc(C#N)ccn3)Nc3cccc(n3)S(=O)(=O)NC2=O)CC1(C)C ; CC(C)(C)OC(=O)NCc1ccnc(C2CC[C@@H]3CN(c4nc(C(C)(C)C)ccc4C(=O)NS(=O)(=O)c4cccc(n4)N2)C(C)(C)C3)c1 ; CC(C)(C)OC(=O)NCc1ccnc(C2CC[C@@H]3CN(c4nc(C(C)(C)C)ccc4C(=O)NS(=O)(=O)c4cccc(n4)N2)C(C)(C)C3)c1]

To a solution of 2-{8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl}pyridine-4-carbonitrile (diastereomeric mixture, 40 mg, 0.06972 mmol) in MeOH (2 mL) was added NiCl₂.H₂O (23 mg, 0.1558 mmol) and cooled in ice bath. To the reaction vial, sodium borohydride (14 mg, 0.3700 mmol) was added slowly (vigorous reaction) and the reaction was allowed to stir at 0° C. for 15 min, then tert-butoxycarbonyl tert-butyl carbonate (34 mg, 0.1558 mmol) was added and the reaction mixture was stirred for 4 h at rt. The reaction mixture was filtered through celite, diluted with water, and extracted with ethyl acetate (3×). The organic layer was isolated, dried over anhydrous Na₂SO₄, filtered, and evaporated. The resultant residue was dissolved in DMSO (1 mL) and filtered through Whatman filter disc (puradisc 25 TF) and the filtrate was purified by a reverse phase HPLC-MS method using a dual gradient run of 30-99% mobile phase B over 15.0 minutes (mobile phase A=H₂O (5 mM HCl) mobile phase B=CH₃CN) to afford two isomers:

First eluting peak-(PEAK-1), diastereomer 1: tert-Butyl N-({2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}methyl)carbamate (hydrochloride salt) (16 mg, 59%). ESI-MS m/z calc. 677.33594, found 678.4 (M+1)⁺; Retention time: 1.1 minutes (LC method G).

Second eluting peak (PEAK-2), diastereomer 2: tert-Butyl N-({2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}methyl)carbamate (hydrochloride salt) (21 mg, 84%). ESI-MS m/z calc. 677.33594, found 678.5 (M+1)⁺; Retention time: 1.18 minutes (LC method G).

### Step 2: (14S)-17-[4-(aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, Compound 116 (diastereomer 1) and (14S)-17-[4-(aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, Compound 115 (diastereomer 2)

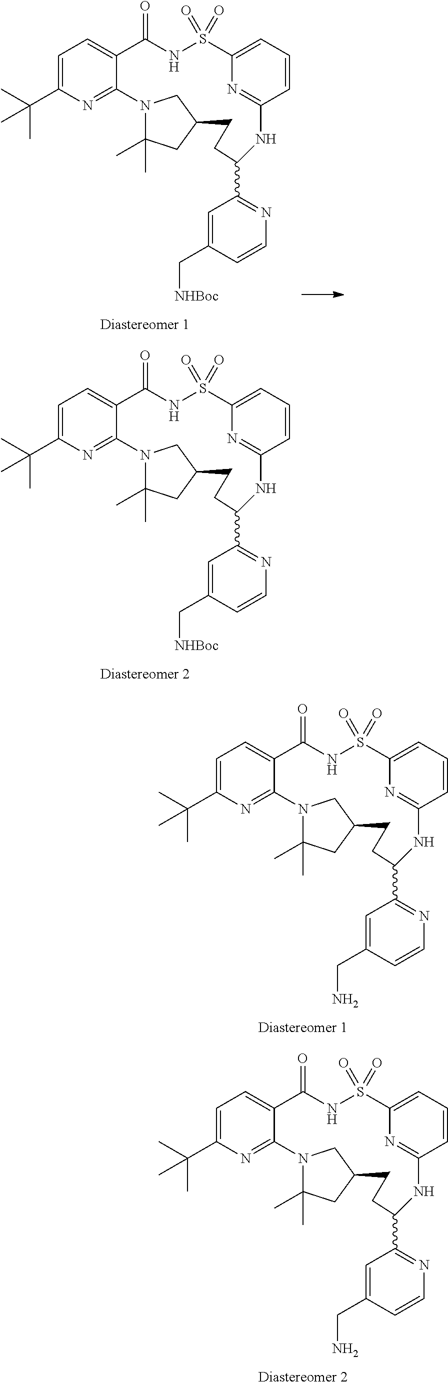 [SMILES: CC(C)(C)OC(=O)NCc1ccnc(C2CC[C@@H]3CN(c4nc(C(C)(C)C)ccc4C(=O)NS(=O)(=O)c4cccc(n4)N2)C(C)(C)C3)c1 ; CC(C)(C)OC(=O)NCc1ccnc(C2CC[C@@H]3CN(c4nc(C(C)(C)C)ccc4C(=O)NS(=O)(=O)c4cccc(n4)N2)C(C)(C)C3)c1 ; CC(C)(C)c1ccc2c(n1)N1C[C@@H](CCC(c3cc(CN)ccn3)Nc3cccc(n3)S(=O)(=O)NC2=O)CC1(C)C ; CC(C)(C)c1ccc2c(n1)N1C[C@@H](CCC(c3cc(CN)ccn3)Nc3cccc(n3)S(=O)(=O)NC2=O)CC1(C)C]

Diastereomer 1: A solution of tert-butyl N-({2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}methyl)carbamate (hydrochloride salt) (Compound 120 (diastereomer 1), 13 mg, 0.01674 mmol) in (premixed solution of 1:3 TFA-DCM) TFA (25 μL, 0.3245 mmol), DCM (75 μL) was stirred at rt for about 2 h and the the solvent was evaporated and the material was dried the material on the lyophilizer overnight to afford (14S)-17-[4-(aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (dihydrochloride salt) (diastereomer 1, 12 mg, 80%). ¹H NMR (400 MHz, DMSO-d₆) δ 12.75 (s, 1H), 8.78 (dd, J=21.2, 15.5 Hz, 3H), 8.60-8.24 (m, 1H), 7.98 (d, J=20.9 Hz, 1H), 7.82 (s, 1H), 7.66 (td, J=8.0, 4.3 Hz, 1H), 7.45 (d, J=7.8 Hz, 1H), 7.28 (t, J=6.9 Hz, 1H), 6.91 (dd, J=8.5, 5.7 Hz, 1H), 6.63 (dd, J=8.0, 1.7 Hz, 1H), 4.95 (s, 1H), 4.23 (d, J=5.8 Hz, 2H), 2.85-2.66 (m, 1H), 2.31 (d, J=15.1 Hz, 1H), 1.93 (d, J=19.4 Hz, 1H), 1.85 (dd, J=11.8, 5.5 Hz, 1H), 1.74 (s, 1H), 1.55 (m, 8H), 1.27 (m, 10H), 1.23-1.07 (m, 1H). ESI-MS m/z calc. 577.2835, found 578.3 (M+1)⁺; Retention time: 1.36 minutes (LC method A).

Diastereomer 2: A solution of tert-butyl N-({2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}methyl)carbamate (hydrochloride salt) (Compound 119 (diastereomer 2), 18.00 mg, 0.02318 mmol) in (premixed solution of 1:3 TFA-DCM) TFA (25 μL, 0.3245 mmol), DCM (75 μL) was stirred at rt for about 2 h and the the solvent was evaporated the material was dried on the lyophilizer overnight to afford (14S)-17-[4-(aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (dihydrochloride salt) (18 mg, 116%). ¹H NMR (400 MHz, DMSO-d₆) δ 12.57 (s, 1H), 8.85-8.78 (m, 3H), 8.08 (s, 1H), 7.92 (d, J=8.4 Hz, 1H), 7.81 (s, 1H), 7.71 (dd, J=8.5, 7.3 Hz, 1H), 7.63 (d, J=8.0 Hz, 1H), 7.18 (d, J=7.2 Hz, 1H), 6.99 (d, J=8.4 Hz, 1H), 6.66 (d, J=8.0 Hz, 1H), 5.44 (q, J=7.9 Hz, 1H), 4.22 (d, J=6.0 Hz, 2H), 3.32 (t, J=8.5 Hz, 1H), 2.71 (t, J=10.2 Hz, 1H), 2.38 (s, 1H), 2.02 (d, J=9.8 Hz, 2H), 1.88-1.74 (m, 2H), 1.66 (s, 3H), 1.62-1.42 (m, 5H), 1.29 (s, 9H). ESI-MS m/z calc. 577.2835, found 578.3 (M+1)⁺; Retention time: 1.49 minutes (LC method A).

### Step 3: Methyl N-({2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}methyl)carbamate, Compound 108 (diastereomer 1) and methyl N-({2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}methyl)carbamate, Compound 107 (diastereomer 2)

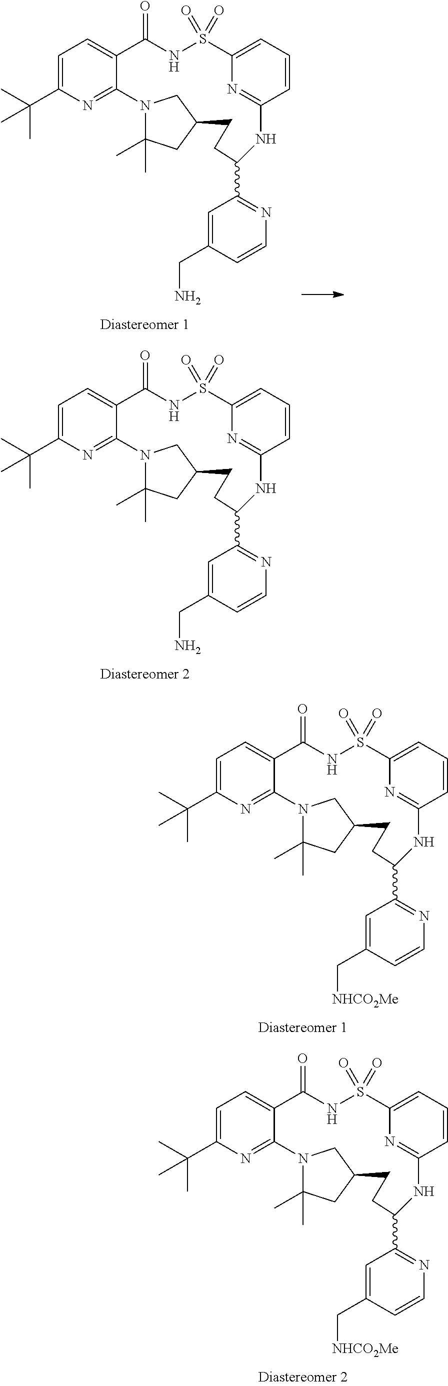

Diastereomer 1: To a solution of (14S)-17-[4-(aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (dihydrochloride salt) (Compound 116 (diastereomer 1), 5 mg, 0.007684 mmol) in DCM (0.5 mL) was added TEA (8 μL, 0.05740 mmol) and cooled in an ice bath. Then, methyl chloroformate (1 μL, 0.01294 mmol) was added slowly and the reaction was allowed to stir for 1 h and then the solvent was evaporated. The resultant residue was dissolved in DMSO (1 mL) and filtered through Whatman filter disc (puradisc 25 TF) and filtrate was purified by a reverse phase HPLC-MS method using a dual gradient run from 30-99% mobile phase B over 15.0 minutes. Mobile phase A=H₂O (5 mM HCl). Mobile phase B=CH₃CN. The solvent was evaporated by lyophilizer methyl N-({2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}methyl)carbamate (hydrochloride salt) (diastereomer 1, 1.4 mg, 26%). ESI-MS m/z calc. 635.289, found 636.1 (M+1)⁺; Retention time: 1.56 minutes (LC method A).

Diastereomer 2: To a solution of (14S)-17-[4-(aminomethyl)pyridin-2-yl]-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (dihydrochloride salt) (Compound 115 (diastereomer 2), 12 mg, 0.01844 mmol) in DCM (0.5 mL) was added TEA (8 μL, 0.05740 mmol) and cooled in ice bath. Then, methyl chloroformate (1 μL, 0.01294 mmol) was added slowly and the reaction was allowed to stir for 1 h and solvent was evaporated. The resultant residue was dissolved in DMSO (1 mL) and filtered through Whatman filter disc (puradisc 25 TF) and filtrate was purified by a reverse phase HPLC-MS method using a dual gradient run of 30-99% mobile phase B over 15.0 minutes (mobile phase A=H₂O (5 mM HCl), mobile phase B=CH₃CN). The solvent was evaporated by lyophilizer to give methyl N-({2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaen-17-yl]pyridin-4-yl}methyl)carbamate (hydrochloride salt) (diastereomer 2, 4.1 mg, 76%) as a white solid. ¹H NMR (400 MHz, DMSO-d₆) δ 12.50 (s, 1H), 8.71 (s, 1H), 7.89 (m, 3H), 7.74 (td, J=7.9, 3.4 Hz, 1H), 7.63 (d, J=8.0 Hz, 2H), 7.21 (dd, J=7.2, 4.0 Hz, 1H), 6.98 (dd, J=8.4, 3.3 Hz, 1H), 6.65 (d, J=8.0 Hz, 1H), 5.46 (s, 1H), 4.39 (s, 2H), 3.56 (s, 3H), 3.33 (s, 1H), 2.67 (t, J=10.2 Hz, 1H), 2.41 (s, 1H), 2.09-1.91 (m, 2H), 1.88-1.71 (m, 2H), 1.67 (s, 3H), 1.61-1.38 (m, 5H), 1.28 (s, 9H). ESI-MS m/z calc. 635.289, found 636.2 (M+1)⁺; Retention time: 1.64 minutes (LC method A).

### Example 45: Preparation of methyl 4-{2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-4-yl}piperidine-1-carboxylate, Compound 96

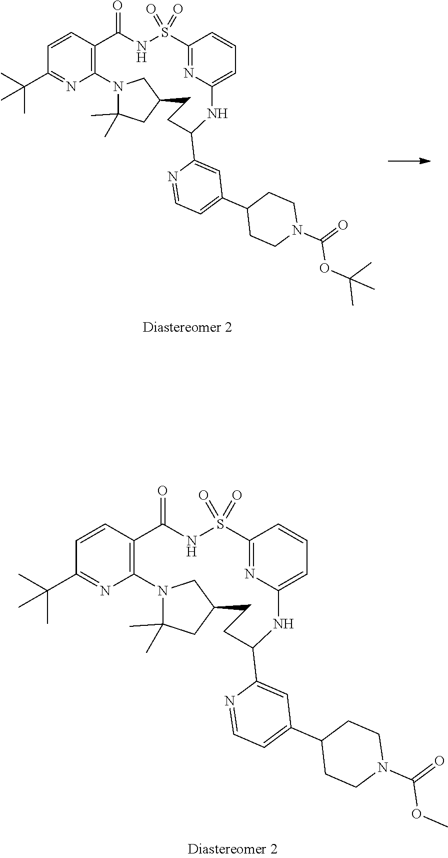

To a solution of tert-butyl 4-{2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-4-yl}piperidine-1-carboxylate (hydrochloride salt) (Compound 117 (diastereomer 2), 10 mg, 0.01301 mmol) in DCM (100 μL) was added TFA (5 μL, 0.06490 mmol) and the mixture was stirred for a couple of hours and the solvents were removed under high vacum. The residue was dissolved in DCM (500 mL), TEA (20 μL, 0.1435 mmol) and cooled in an ice bath. Then, methyl chloroformate (1.3 μL, 0.01682 mmol) was added slowly and the reaction was allowed to stir for 1 h and the solvent was evaporated. The resultant residue was dissolved in DMSO (1 mL) and filtered through Whatman filter disc (puradisc 25 TF) and filtrate was purified by a reverse phase HPLC-MS method using a dual gradient run of 20-80% mobile phase B over 15.0 minutes (mobile phase A=H₂O (5 mM HCl), mobile phase B=CH₃CN) to afford methyl 4-{2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]pyridin-4-yl}piperidine-1-carboxylate (hydrochloride salt) (2.2 mg, 23%) as a white solid. ESI-MS m/z calc. 689.33594, found 690.34 (M+1)⁺; Retention time: 1.06 minutes (LC method G).

### Example 46: Preparation of (14S)-17-(4-Bromopyridin-2-yl)-8-tert-butyl-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, diastereomer 1, Compound 47 and diastereomer 2, Compound 46

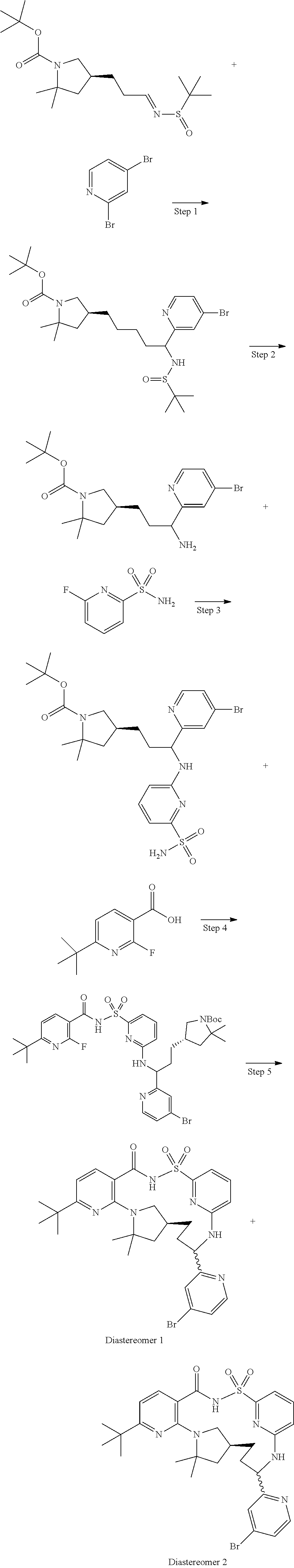

### Step 1: tert-Butyl (4S)-4-[3-(4-bromo-2-pyridyl)-3-(tert-butylsulfinylamino)propyl]-2,2-dimethyl-pyrrolidine-1-carboxylate

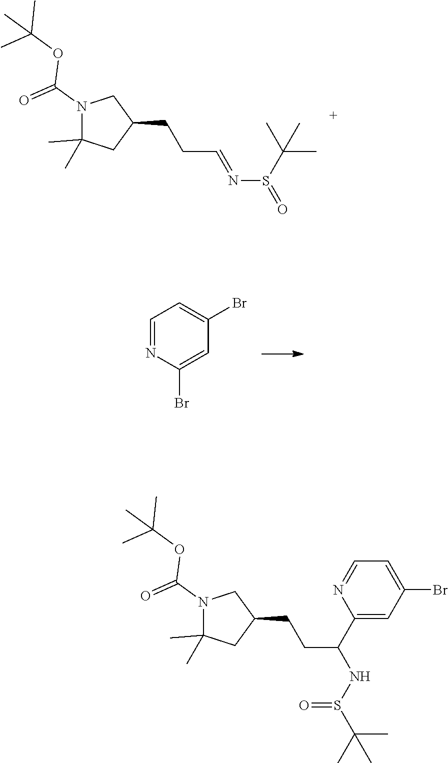

To a stirring solution of 2,4-dibromopyridine (9.29 g, 39.216 mmol) in anhydrous toluene (225 mL) at −78° C. under nitrogen was dropwise added a solution of n-butyllithium (14 mL of 2.5 M in hexanes, 35.000 mmol). After the addition was complete, the reaction mixture was stirred at this temperature for 2 h. To the reaction mixture was promptly added via syringe a solution of tert-butyl (4S)-4-[(3E)-3-tert-butylsulfinyliminopropyl]-2,2-dimethyl-pyrrolidine-1-carboxylate (10.692 g, 29.821 mmol) in anhydrous toluene (55 mL). After the addition was complete, the reaction mixture was stirred at −78° C. for 1 hour. The reaction was slowly quenched cold with a saturated aqueous NH₄Cl (150 mL), and then allowed to warm up to room temperature. The reaction mixture was poured into a mixture of brine (100 mL) and ethyl acetate (100 mL), and two layers were separated. The aqueous layer was extracted with ethyl acetate (2×150 mL). Combined organic layers were washed with brine (100 mL), dried over anhydrous sodium sulfate and concentrated. The crude was purified by silica gel chromatography using 0-25% acetone gradient in hexanes to afford tert-butyl (4S)-4-[3-(4-bromo-2-pyridyl)-3-(tert-butylsulfinylamino)propyl]-2,2-dimethyl-pyrrolidine-1-carboxylate (11.739 g, 72%) as a white foam. ESI-MS m/z calc. 515.1817, found 516.3 (M+1)⁺; Retention time: 5.85 minutes (LC method C).

### Step 2: tert-Butyl (4S)-4-[3-amino-3-(4-bromo-2-pyridyl)propyl]-2,2-dimethyl-pyrrolidine-1-carboxylate

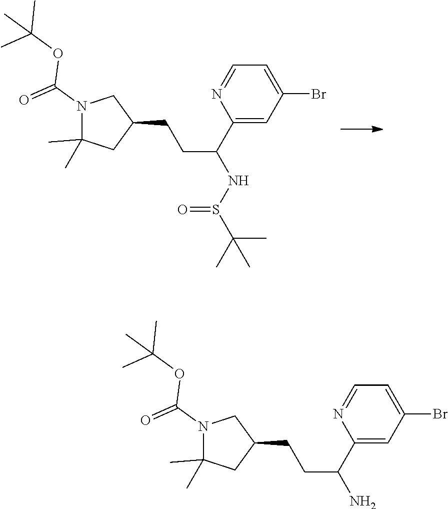 [SMILES: CC(C)(C)OC(=O)N1C[C@@H](CCC(NS(=O)C(C)(C)C)c2cc(Br)ccn2)CC1(C)C ; CC(C)(C)OC(=O)N1C[C@@H](CCC(N)c2cc(Br)ccn2)CC1(C)C]

To a stirring solution of tert-butyl (4S)-4-[3-(4-bromo-2-pyridyl)-3-(tert-butylsulfinylamino)propyl]-2,2-dimethyl-pyrrolidine-1-carboxylate (11.739 g, 22.726 mmol) in a mixture of THF (120 mL) and water (30 mL) at room temperature under ambient conditions was added iodine (2.162 g, 8.5182 mmol). The reaction mixture was heated to 55° C. for 2 h. After cooling to room temperature, the reaction mixture was poured into a mixture of saturated aqueous sodium bicarbonate (200 mL) and saturated aqueous Na₂S₂O₃ (50 mL). Volatiles were removed under vacuum, and the residual aqueous layer was extracted with ethyl acetate (3×150 mL). The combined organic layers were washed with brine (80 mL), dried over anhydrous sodium sulfate and concentrated to afford tert-butyl (4S)-4-[3-amino-3-(4-bromo-2-pyridyl)propyl]-2,2-dimethyl-pyrrolidine-1-carboxylate (10.592 g, 102%) as amber oil. The product was carried to the next step without further purification. ESI-MS m/z calc. 411.1521, found 412.3 (M+1)⁺; Retention time: 4.33 minutes (LC method C).

### Step 3: tert-Butyl (4S)-4-[3-(4-bromo-2-pyridyl)-3-[(6-sulfamoyl-2-pyridyl)amino]propyl]-2,2-dimethyl-pyrrolidine-1-carboxylate

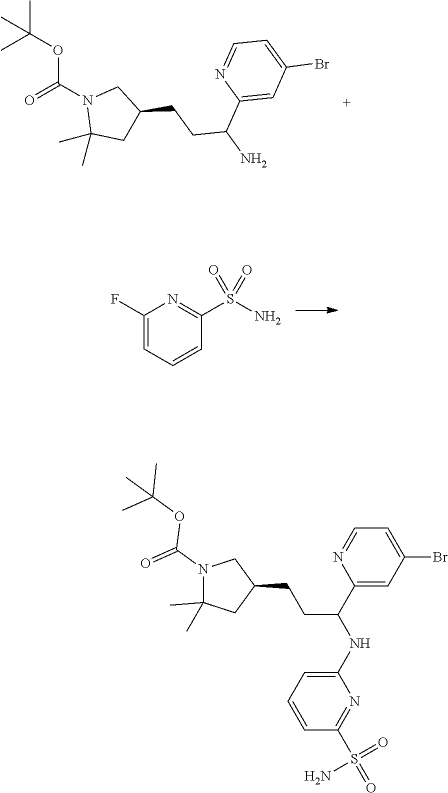 [SMILES: CC(C)(C)OC(=O)N1C[C@@H](CCC(N)c2cc(Br)ccn2)CC1(C)C ; NS(=O)(=O)c1cccc(F)n1 ; CC(C)(C)OC(=O)N1C[C@@H](CCC(Nc2cccc(S(N)(=O)=O)n2)c2cc(Br)ccn2)CC1(C)C]

To a stirring solution of tert-butyl (4S)-4-[3-amino-3-(4-bromo-2-pyridyl)propyl]-2,2-dimethyl-pyrrolidine-1-carboxylate (10.592 g, 23.117 mmol) and 6-fluoropyridine-2-sulfonamide (5.964 g, 33.854 mmol) in anhydrous DMSO (40 mL) at room temperature under nitrogen was added DIEA (12.4 mL, 71.190 mmol). The reaction mixture was heated to 125° C. for 24 h. After cooling to room temperature, the reaction mixture was poured into a mixture of water (200 mL) and brine (300 mL). The product was extracted with ethyl acetate (3×200 mL). Combined organic layers were washed with brine (100 mL), dried over anhydrous sodium sulfate and concentrated. The crude was purified by the reverse phase HPLC using 45-85% acetonitrile gradient in water (0.15% TFA buffer; C₁₈ Varian column; 60 mL/min.). All fractions containing the purified product were combined and basified with saturated aqueous sodium bicarbonate to pH˜8. Volatiles were removed under vacuum, and the residual aqueous layer was extracted with ethyl acetate (3×250 mL). The combined organic layers were washed with brine (120 mL), dried over anhydrous sodium sulfate and concentrated to afford tert-butyl (4S)-4-[3-(4-bromo-2-pyridyl)-3-[(6-sulfamoyl-2-pyridyl)amino]propyl]-2,2-dimethyl-pyrrolidine-1-carboxylate (6.779 g, 47%) as pale yellow solid. ¹H NMR (500 MHz, DMSO-d₆) δ 8.42 (d, J=5.2 Hz, 1H), 7.80-7.75 (m, 1H), 7.57-7.45 (m, 3H), 7.04 (s, 2H), 6.97 (d, J=7.2 Hz, 1H), 6.71 (d, J=8.5 Hz, 1H), 5.20 (s, 1H), 3.60-3.45 (m, 1H), 2.83-2.66 (m, 1H), 2.16-2.01 (m, 1H), 1.94-1.74 (m, 3H), 1.41-1.35 (m, 11H), 1.35-1.30 (m, 4H), 1.22 (s, 3H). ESI-MS m/z calc. 567.1515, found 568.3 (M+1)⁺; Retention time: 2.37 minutes (LC method B).

### Step 4: tert-Butyl (4S)-4-[3-(4-bromo-2-pyridyl)-3-[[6-[(6-tert-butyl-2-fluoro-pyridine-3-carbonyl)sulfamoyl]-2-pyridyl]amino]propyl]-2,2-dimethyl-pyrrolidine-1-carboxylate

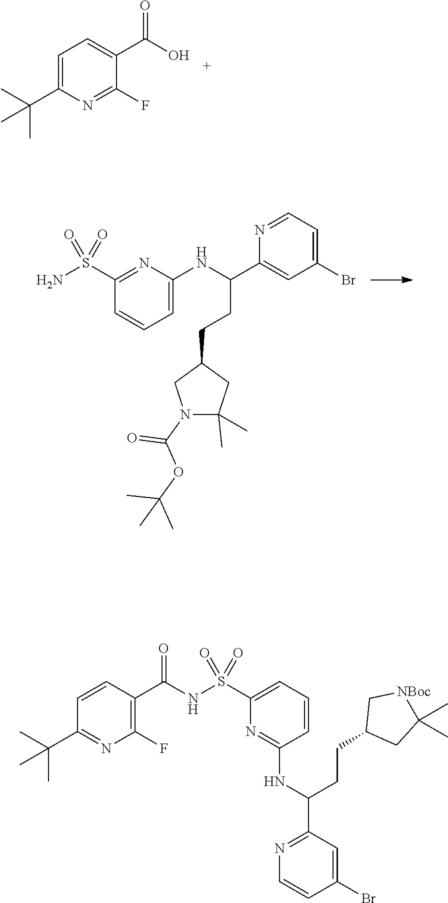 [SMILES: CC(C)(C)c1ccc(C(=O)O)c(F)n1 ; CC(C)(C)OC(=O)N1C[C@@H](CCC(Nc2cccc(S(N)(=O)=O)n2)c2cc(Br)ccn2)CC1(C)C ; CC(C)(C)OC(=O)N1C[C@@H](CCC(Nc2cccc(S(=O)(=O)NC(=O)c3ccc(C(C)(C)C)nc3F)n2)c2cc(Br)ccn2)CC1(C)C]

In a 100-mL round-bottomed flask, 6-tert-butyl-2-fluoro-pyridine-3-carboxylic acid (1.483 g, 7.520 mmol) was dissolved in THF (30 mL), to which CDI (1.210 g, 7.462 mmol) was added. The resulting mixture was stirred at room temperature for 14 h. After this time, tert-butyl (4S)-4-[3-(4-bromo-2-pyridyl)-3-[(6-sulfamoyl-2-pyridyl)amino]propyl]-2,2-dimethyl-pyrrolidine-1-carboxylate (2.4958 g, 4.241 mmol) and DBU (3 mL, 20.06 mmol) were added, and the resulting mixture was stirred at room temperature for 24 h. After this time, the mixture was concentrated in vacuo. Then, the mixture was poured into EtOAc (300 mL). This mixture was then washed with saturated aqueous sodium bicarbonate solution (150 mL), aqueous HCl solution (0.1 N, 150 mL) and saturated aqueous NaCl solution (150 mL), then dried over Na₂SO₄, filtered, and evaporated in vacuo. The resulting brown foam was purified by silica gel chromatography (120 g of silica) using a gradient eluent of 0 to 80% EtOAc in hexanes to give an off-white foam, tert-butyl (4S)-4-[3-(4-bromo-2-pyridyl)-3-[[6-[(6-tert-butyl-2-fluoro-pyridine-3-carbonyl)sulfamoyl]-2-pyridyl]amino]propyl]-2,2-dimethyl-pyrrolidine-1-carboxylate (1.7446 g, 55%); ESI-MS m/z calc. 746.22614, found 747.2 (M+1)⁺; Retention time: 2.13 minutes and 2.15 minutes (LC method A).

### Step 5: (14S)-17-(4-Bromopyridin-2-yl)-8-tert-butyl-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, diastereomer 1, Compound 47 and (14S)-17-(4-bromopyridin-2-yl)-8-tert-butyl-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, diastereomer 2, Compound 46

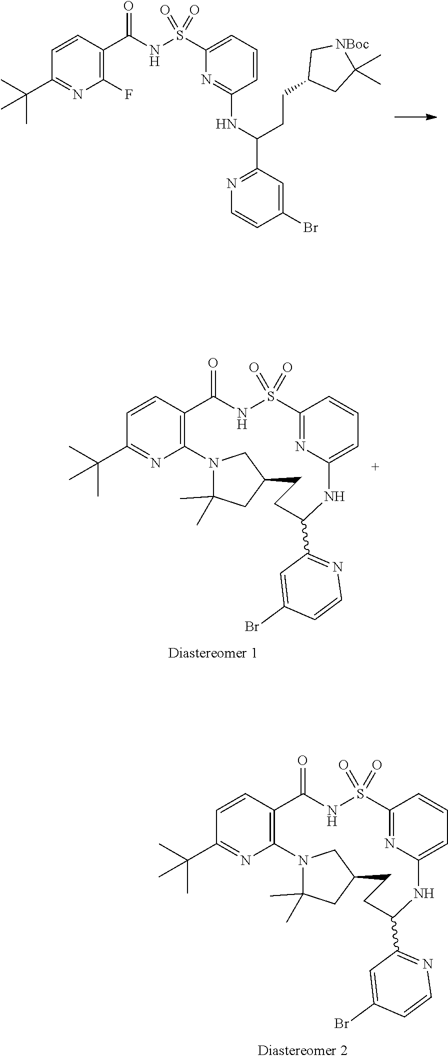

Stage 1: In a 100-mL round-bottomed flask, tert-butyl (4S)-4-[3-(4-bromo-2-pyridyl)-3-[[6-[(6-tert-butyl-2-fluoro-pyridine-3-carbonyl)sulfamoyl]-2-pyridyl]amino]propyl]-2,2-dimethyl-pyrrolidine-1-carboxylate (1.7446 g, 2.333 mmol), was dissolved in dichloromethane (30 mL). TFA (4.0 mL, 51.92 mmol) was added, and the resulting solution was allowed to stand at room temperature for 24 h. The mixture was then evaporated in vacuo, diluted with dioxane, and evaporated in vacuo again. This gave an orange foam, 1.8 g (>100% yield). Stage 2: In a 100-mL round-bottomed flask, the crude product from Step 1 was dissolved in NMP (30 mL), to which K₂CO₃ (3.0 g, 21.71 mmol) was added. The resulting mixture was flushed with nitrogen, then stirred at 150° C. for 18 h. After cooling to room temperature, the reaction mixture was poured into an aqueous HCl solution (1 N; 200 mL), then extracted with EtOAc (2×200 mL). The combined organic extracts were washed with H₂O (200 mL) and saturated aqueous NaCl solution (200 mL), then dried over Na₂SO₄, filtered, and evaporated in vacuo. A brown foam (˜3 g) was obtained as the crude product. Several purifications by silica gel chromatography gave two separate products.

Diastereomer 1, “Peak 1”: (14S)-17-(4-Bromopyridin-2-yl)-8-tert-butyl-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (300.9 mg, 21%); ¹H NMR (400 MHz, DMSO-d₆) δ 12.31 (s, 1H), 8.44 (d, J=5.2 Hz, 1H), 7.83 (s, 1H), 7.75 (d, J=8.0 Hz, 1H), 7.65 (t, J=7.9 Hz, 1H), 7.56 (d, J=4.5 Hz, 1H), 7.49 (d, J=6.6 Hz, 1H), 7.23 (d, J=7.1 Hz, 1H), 6.86 (d, J=8.5 Hz, 1H), 6.63 (d, J=7.9 Hz, 1H), 5.10-4.84 (m, 1H), 3.18-3.05 (m, 1H), 3.05-2.89 (m, 1H), 2.40-2.03 (m, 3H), 1.85 (dd, J=11.9, 5.9 Hz, 1H), 1.78-1.67 (m, 1H), 1.60 (s, 3H), 1.57-1.53 (m, 1H), 1.52 (s, 3H), 1.27 (s, 9H), 1.25-1.13 (m, 1H). ESI-MS m/z calc. 626.1675, found 627.2 (M+1)⁺; Retention time: 2.0 minutes (LC method A).

Diastereomer 2, “Peak 2”: (14S)-17-(4-Bromopyridin-2-yl)-8-tert-butyl-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (438.0 mg, 30%); ¹H NMR (400 MHz, DMSO-d₆) δ 12.49 (s, 1H), 8.42 (d, J=5.3 Hz, 1H), 7.81-7.58 (m, 4H), 7.53 (dd, J=5.3, 1.9 Hz, 1H), 7.18-7.03 (m, 1H), 6.95-6.80 (m, 1H), 6.70-6.57 (m, 1H), 5.33-5.18 (m, 1H), 3.26-3.15 (m, 1H), 2.80-2.68 (m, 1H), 2.29-2.15 (m, 1H), 1.97-1.85 (m, 2H), 1.83 (dd, J=11.5, 4.8 Hz, 1H), 1.77-1.68 (m, 1H), 1.64 (s, 3H), 1.62-1.53 (m, 1H), 1.50 (s, 3H), 1.48-1.39 (m, 1H), 1.28 (s, 9H). ESI-MS m/z calc. 626.1675, found 627.2 (M+1)⁺; Retention time: 2.03 minutes (LC method A).

### Example 47: Preparation of (14S)-8-tert-butyl-17-[4-(2-hydroxypropan-2-yl)pyridin-2-yl]-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, Compound 36

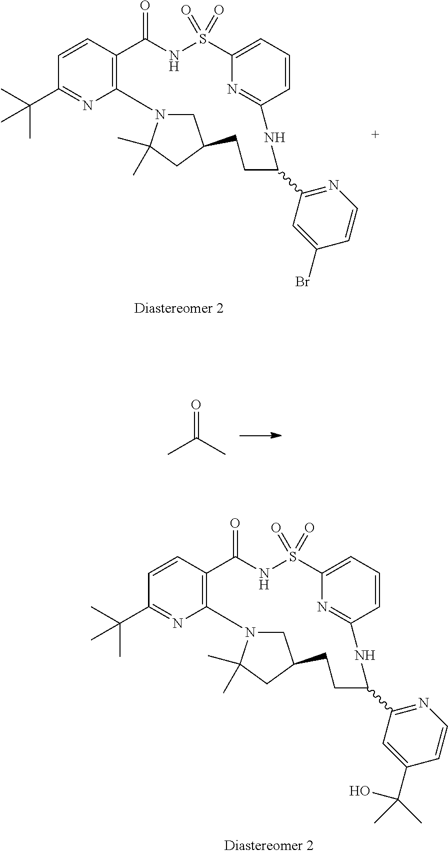

In a 1-mL microwave vial, (14S)-17-(4-bromopyridin-2-yl)-8-tert-butyl-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (hydrochloride salt) (Diastereomer 2, Compound 46, 18 mg, 0.02711 mmol) was dissolved in anhydrous THF (300 μL), to which NaH (5 mg of 60% w/w, 0.1250 mmol) was added. The resulting mixture was stirred at room temperature for 15 min. Then, the mixture was cooled to 78° C., to which a pentane solution of tert-butyllithium (40 μL of 1.7 M, 0.06800 mmol) was added. The resulting dark solution was stirred at 78° C. for 15 min, after which molecular-sieve-dried acetone (10 μL, 0.1362 mmol) was added. The resulting mixture was stirred at 78° C. for 15 min, then warmed to room temperature over 30 min. It was quenched with saturated NH₄Cl solution, then diluted with EtOAc (1 mL). The phases were vigorously mixed then separated. The organic layer was then dried over Na₂SO₄ and evaporated in vacuo. The resulting oil was diluted with MeOH (500 μL), filtered, and purified by reverse-phase preparative chromatography using a C₁₈ column and a gradient eluent of 1 to 70% MeCN in H₂O containing 5 mM HCl solution to give 2 products:

(14S)-8-tert-Butyl-17-[4-(2-hydroxypropan-2-yl)pyridin-2-yl]-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (hydrochloride salt) (diastereomer 2, 2.7 mg, 15%); ESI-MS m/z calc. 606.2988, found 607.3 (M+1)⁺; Retention time: 1.59 minutes (LC method A).

Dehalogenated side product: (14S)-8-tert-butyl-12,12-dimethyl-17-(pyridin-2-yl)-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (4.4 mg, 28%); ESI-MS m/z calc. 548.25696, found 549.2 (M+1)⁺; Retention time: 1.6 minutes (LC method A).

### Example 48: Preparation of (14S)-8-tert-butyl-12,12-dimethyl-17-[4-(oxetan-3-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione, Compound 26 (diastereomer 1), and Compound 25 (diastereomer 2)

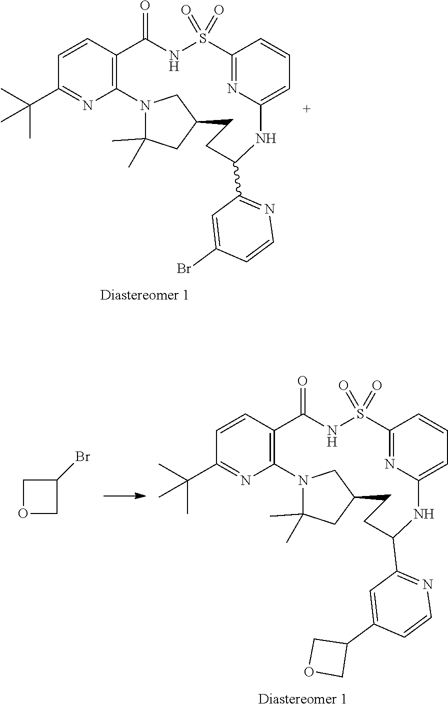 [SMILES: CC(C)(C)c1ccc2c(n1)N1C[C@@H](CCC(c3cc(Br)ccn3)Nc3cccc(n3)S(=O)(=O)NC2=O)CC1(C)C ; BrC1COC1 ; CC(C)(C)c1ccc2c(n1)N1C[C@@H](CCC(c3cc(C4COC4)ccn3)Nc3cccc(n3)S(=O)(=O)NC2=O)CC1(C)C]

In a 1-dram vial, 4-tert-butyl-2-(4-tert-butyl-2-pyridyl)pyridine (2.2 mg, 0.008197 mmol), [Ir{dF(CF₃)ppy}2(dtbpy)]PF₆ (1.0 mg, 8.913E-4 mmol) and NiCl₂-DME (1.8 mg, 0.008192 mmol) were added. The vial was capped with a septum and three vacuum pump/nitrogen purge cycles were performed. Then, anhydrous DME (0.5 mL) and 2,6-dimethylpyridine (30 μL, 0.2590 mmol) were added, and this mixture was stirred at room temperature under nitrogen gas for 10 min to form the ligated nickel (color change from yellow to greenish yellow). After this time, under nitrogen gas sparging, a solution of (14S)-17-(4-bromopyridin-2-yl)-8-tert-butyl-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (diastereomer 1, Compound 47, 35 mg, 0.0558 mmol) in DME (1.0 mL) was added, followed by 3-bromooxetane (30 mg, 0.2190 mmol) and bis(trimethylsilyl)silyl-trimethyl-silane (70 μL, 0.2269 mmol). The resulting yellow transparent solution was stirred under blue light irradiation (Merck Photoreactor, 34 W light source, 100% power, 4700 rpm fan, 1700 rpm stirring) for 2 h. After this time, the mixture was quenched with H₂O (2 mL), and extracted with EtOAc (2×1 mL). The combined organic extracts was washed with H₂O (2 mL) and saturated aqueous NaCl solution (2 mL), then dried over Na₂SO₄, filtered, and evaporated in vacuo. Purification by silica gel chromatography (4 g of silica) using a gradient eluent of 0 to 100% EtOAc in hexanes gave 12 mg of a 80% pure sample. Further purification by preparative TLC (one full silica plate, 20 cm×20 cm, 250 μm thickness, 60 Å particle size, 75% EtOAc: 25% hexanes, UV active band) gave (14S)-8-tert-butyl-12,12-dimethyl-17-[4-(oxetan-3-yl)pyridin-2-yl]-2λ⁶-thia-3,9,11,18,23-pentaazatetracyclo[17.3.1.111,14.05,10]tetracosa-1(23),5(10),6,8,19,21-hexaene-2,2,4-trione, diastereomer 1 (4.9 mg, 13%); ¹H NMR (400 MHz, DMSO-d₆) δ 12.44-12.06 (bs, 1H), 8.52 (d, J=5.1 Hz, 1H), 7.71 (d, J=8.1 Hz, 1H), 7.66-7.55 (m, 2H), 7.54-7.43 (m, 1H), 7.34-7.25 (m, 1H), 7.21 (d, J=7.4 Hz, 1H), 6.85 (d, J=8.5 Hz, 1H), 6.64 (d, J=8.0 Hz, 1H), 5.03-4.78 (m, 3H), 4.62 (td, J=6.4, 3.8 Hz, 2H), 4.28-4.18 (m, 1H), 3.18-2.97 (m, 2H), 2.32-2.24 (m, 1H), 2.23-2.02 (m, 2H), 1.85 (dd, J=12.0, 5.8 Hz, 1H), 1.77-1.65 (m, 1H), 1.60 (s, 3H), 1.52 (s, 3H), 1.52-1.45 (m, 1H), 1.27 (s, 9H). [Note: 1H is missing from the overall proton count of 40H.]. ESI-MS m/z calc. 604.2832, found 605.3 (M+1)⁺; Retention time: 1.49 minutes (LC method A).

The compound in the following tables was prepared in a manner analogous to that described above using (14S)-17-(4-bromopyridin-2-yl)-8-tert-butyl-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, diastereomer 2, Compound 46 as a starting material:

| | | LCMS | | | |
|---|---|---|---|---|---|
| Compound | | Retention | Exact | | LCMS |
| Number | Structure | Time (min) | Mass | M + 1 | Method |
| Compound 25 (diastereomer 2) | 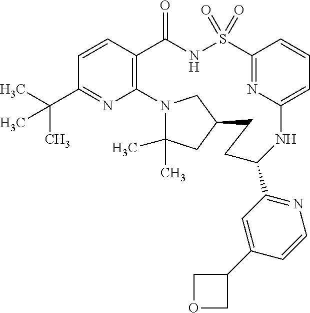 [SMILES: CC(C)(C)c1ccc2c(n1)N1C[C@@H](CC[C@@H](c3cc(C4COC4)ccn3)Nc3cccc(n3)S(=O)(=O)NC2=O)CC1(C)C] | 1.58 | 604.283 | 605.3 | LC method A |

| Compound | |
|---|---|
| Number | NMR |
| Compound | ¹H NMR (400 MHz, DMSO-d₆) δ 12.63-12.23 |
| 25 | (bs, 1H), 8.49 (d, J = 5.0 Hz, 1H), 7.69-7.58 |
| (diastereomer | (m, 3H), 7.42 (d, J = 1.7 Hz, 1H), 7.30 (dd, J = 5.1, |
| 2) | 1.7 Hz, 1H), 7.09 (d, J = 7.2 Hz, 1H), 6.88 (d, J = |
| | 8.5 Hz, 1H), 6.66 (d, J = 8.0 Hz, 1H), 5.34-5.18 (m, |
| | 1H), 4.90 (ddd, J = 8.6, 5.9, 2.8 Hz, 2H), 4.58 (dt, |
| | J = 11.2, 6.3 Hz, 2H), 4.27-4.17 (m, 1H), 3.28-3.17 |
| | (m, 1H), 2.76 (t, J = 10.5 Hz, 1H), 2.28-2.15 (m, 1H), |
| | 1.96-1.87 (m, 2H), 1.84 (dd, J = 11.7, 5.1 Hz, 1H), |
| | 1.78-1.68 (m, 1H), 1.65 (s, 3H), 1.57 (t, J = 12.4 Hz, |
| | 1H), 1.51 (s, 3H), 1.49-1.39 (m, 1H), 1.28 (s, 9H) |

### Example 49: Preparation of (14S)-8-tert-butyl-17-(5-tert-butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 187 (diastereomer 1), and Compound 186 (diastereomer 2)

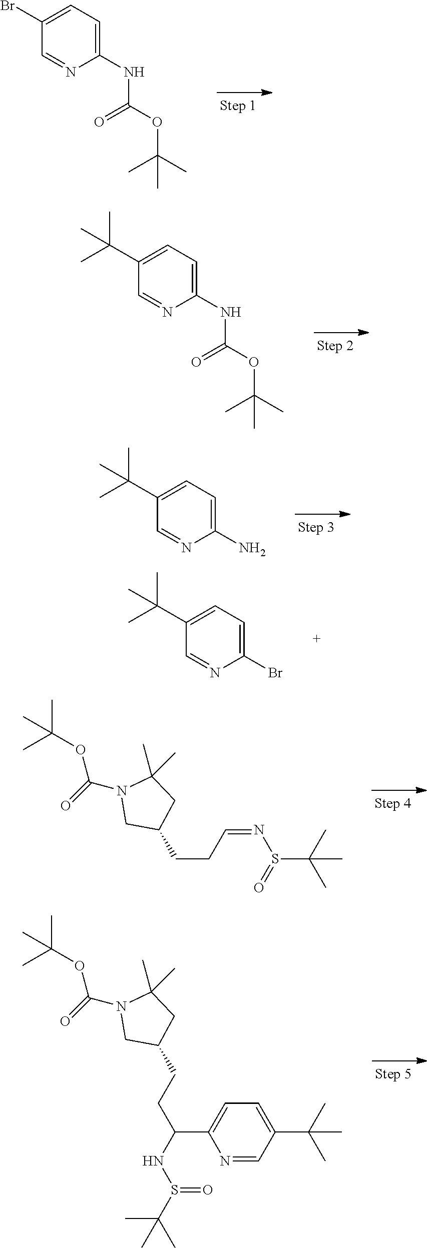

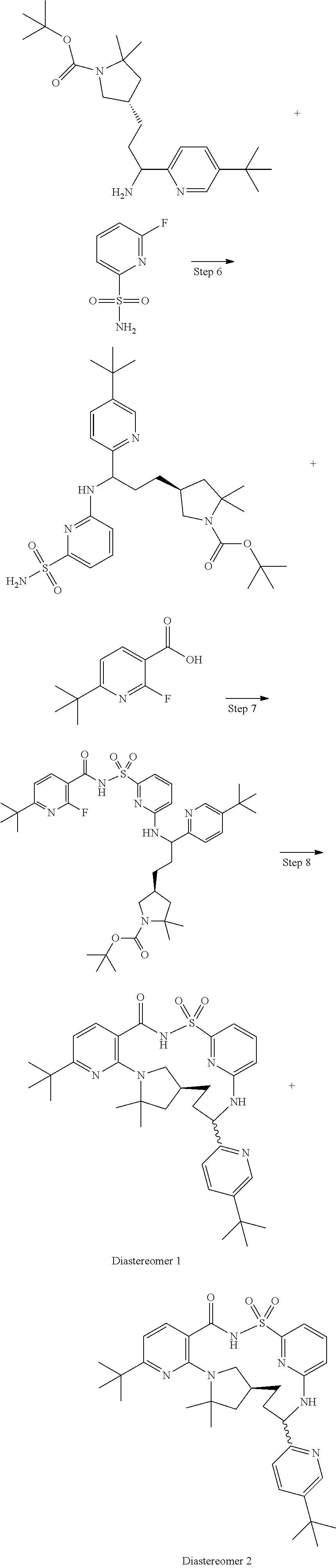

### Step 1: tert-Butyl N-(5-tert-butyl-2-pyridyl)carbamate

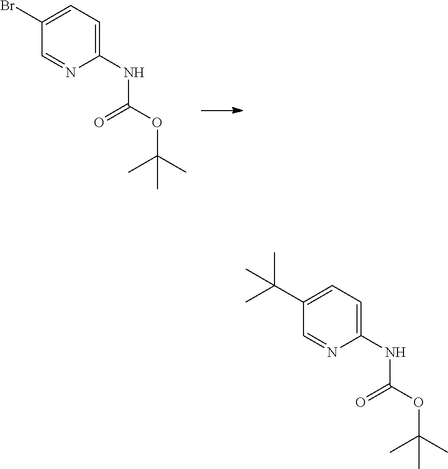 [SMILES: CC(C)(C)OC(=O)Nc1ccc(Br)cn1 ; CC(C)(C)OC(=O)Nc1ccc(C(C)(C)C)cn1]

tert-Butylmagnesium chloride (415 mL of 1.7 M, 705.50 mmol) was added slowly to copper cyanide (31.15 g, 347.80 mmol) in THF (500 mL) at −78° C., and stirred at same temperature for 20 minutes. tert-butyl N-(5-bromo-2-pyridyl)carbamate (24.01 g, 87.908 mmol) in THF (400 mL) was added slowly to reaction mixture, and stirred at −78° C. for 2 h, then stirred at room temperature for overnight. The reaction was quenched with saturated NH₄OH (100 mL), and 1N NaOH (75 mL) at 0° C. The reaction mixture was filtered, and rinsed with diethyl ether (500 mL). The solvent was evaporated until around 300 mL under reduced pressure, and then diluted with diethyl ether (1500 mL), washed with water (720 mL), dried over anhydrous sodium sulfate, filtered, concentrated under vacuum carefully. The residue was purified by silica gel chromatography (twice) using 0 to 20% EtOAc in hexanes to afford tert-butyl N-(5-tert-butyl-2-pyridyl)carbamate (4.71 g, 21%) as pale yellow solid. ESI-MS m/z calc. 250.1681, found 251.4 (M+1)⁺; Retention time: 2.22 minutes (LC method B).

### Step 2: 5-tert-Butylpyridin-2-amine

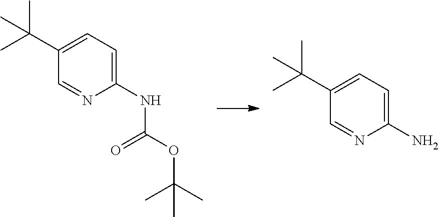 [SMILES: CC(C)(C)OC(=O)Nc1ccc(C(C)(C)C)cn1 ; CC(C)(C)c1ccc(N)nc1]

To a solution of tert-butyl N-(5-tert-butyl-2-pyridyl)carbamate (4.51 g, 18.016 mmol) in DCM (22 mL) at 0° C. was added TFA (22 mL). The reaction mixture was stirred at room temperature for 4 h. The solvent was evaporated by reduced pressure. The residue was diluted with DCM (300 mL), and washed with saturated aqueous sodium bicarbonate (300 mL), dried over anhydrous sodium sulfate and concentrated, dried under vacuum to afford 5-tert-butylpyridin-2-amine (2.69 g, 99%) as a yellow solid. ESI-MS m/z calc. 150.1157, found 151.2 (M+1)⁺; Retention time: 1.59 minutes (LC method B).

### Step 3: 2-bromo-5-tert-butyl-pyridine

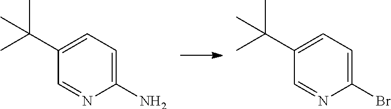

To a suspension of 5-tert-butylpyridin-2-amine (2.7 g, 17.974 mmol) in HBr (26 mL of 48% w/w, 110.31 mmol) was added Bra (4.5 mL, 87.349 mmol) dropwise at −20° C. and the reaction was stirred at the same temperature for 1.5 h. Then a solution of sodium nitrite (5.75 g, 83.339 mmol) in water (8.6 mL) was added dropwise to the reaction mixture. The reaction mixture was allowed to warm to 15° C. over 1 hour, and stirred at same temperature for 30 minutes. The reaction mixture was cooled to −20° C., and treated with aqueous NaOH (10N, 80 mL). The reaction mixture was stirred at room temperature for 10 minutes, then extracted with diethyl ether (3×135 mL). The combined organic layers were washed with water (150 mL), dried over anhydrous sodium sulfate and concentrated, dried under vacuum to afford 2-bromo-5-tert-butyl-pyridine (3.7094 g, 96%) as brown oil. ESI-MS m/z calc. 213.0153, found 214.3 (M+1)⁺; Retention time: 3.22 minutes ¹H NMR (250 MHz, DMSO-d₆) δ 8.43 (d, J=2.8 Hz, 1H), 7.78 (dd, J=8.3, 2.7 Hz, 1H), 7.55 (dd, J=8.4, 0.7 Hz, 1H), 1.29 (s, 9H). ESI-MS m/z calc. 213.0153, found 214.3 (M+1)⁺; Retention time: 3.22 minutes (LC method B).

### Step 4: tert-Butyl (4S)-4-[3-(5-tert-butyl-2-pyridyl)-3-(tert-butylsulfinylamino)propyl]-2,2-dimethyl-pyrrolidine-1-carboxylate

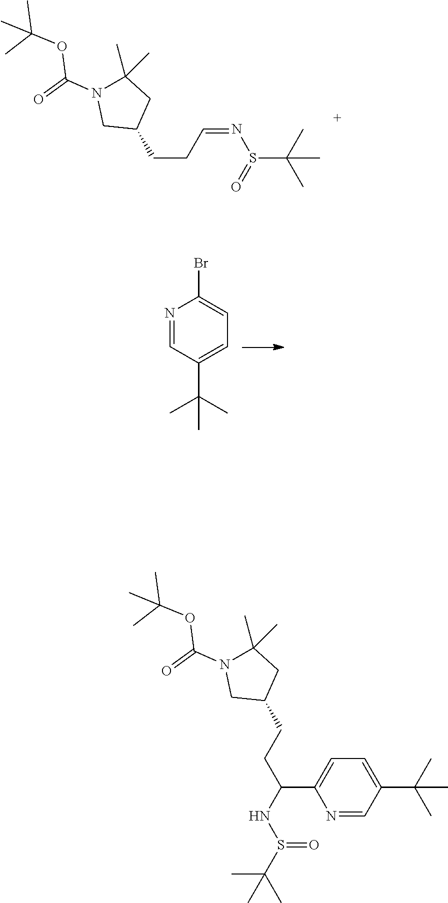

2-Bromo-5-tert-butyl-pyridine (1.7 g, 7.5431 mmol) was dissolved in diethyl ether (40 mL) and the solution was cooled in a dry ice acetone bath (<−70° C.) under a nitrogen balloon. n-BuLi (3.1 mL of 2.5 M in hexanes, 7.7500 mmol) was added. The mixture was stirred at this temperature for 1 h. tert-butyl (4S)-4-[(3Z)-3-tert-butylsulfinyliminopropyl]-2,2-dimethyl-pyrrolidine-1-carboxylate (3.33 g, 8.8233 mmol) was added as a THF (8 mL) solution. The mixture was stirred at −78° C. to −30° C. for 1 h. Saturated aqueous NH₄Cl (25 mL) was added. The mixture was allowed to warmed up to rt and partitioned between water (50 mL) and EtOAc (100 mL). The layers were separated and the aqueous layer was extracted with more EtOAc (100 mL). The combined organic layer was washed with brine, dried over sodium sulfate and concentrated. The product was combined with another batch prepared using the same chemistry on a 330 mg scale to afford crude tert-butyl (4S)-4-[3-(5-tert-butyl-2-pyridyl)-3-(tert-butylsulfinylamino)propyl]-2,2-dimethyl-pyrrolidine-1-carboxylate (5.5 g, 99% adjusted yield). ESI-MS m/z calc. 493.3338, found 494.6 (M+1)⁺; Retention time: 3.12 minutes (LC method B).

### Step 5: tert-Butyl (4S)-4-[3-amino-3-(5-tert-butyl-2-pyridyl)propyl]-2,2-dimethyl-pyrrolidine-1-carboxylate

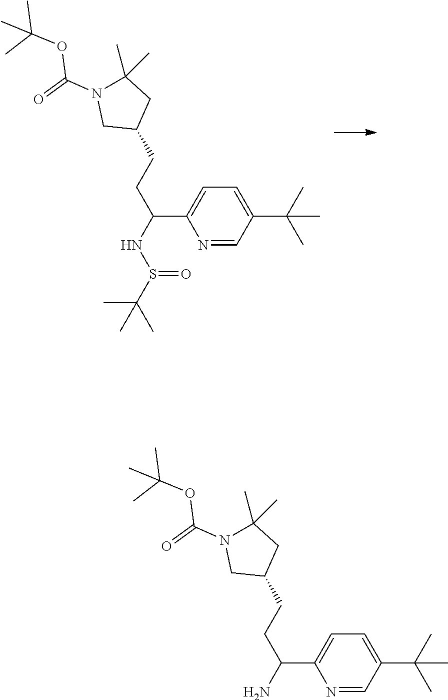

tert-Butyl (4S)-4-[3-(5-tert-butyl-2-pyridyl)-3-(tert-butylsulfinylamino)propyl]-2,2-dimethyl-pyrrolidine-1-carboxylate (5.5 g, 8.9115 mmol) was dissolved in THF (52 mL) and water (10 mL). Molecular iodine (700 mg, 2.7525 mmol) was added. The mixture was stirred at 35° C. for 16 h. It was then cooled to rt and partitioned between EtOAc (200 mL) and Na₂S₂O₃ (60 g) in saturated aqueous sodium bicarbonate (200 mL). The layers were separated and the aqueous layer was extracted once with EtOAc (100 mL). The organic layer was concentrated. The residue was dissolved in 1M HCl (400 mL) and was extracted with EtOAc (300 mL). The aqueous layer was basified by 2.5M NaOH and extracted with EtOAc (2×300 mL). The organic layer was washed with brine, dried over anhydrous Na₂SO₄, filtered and concentrated to give tert-butyl (4S)-4-[3-amino-3-(5-tert-butyl-2-pyridyl)propyl]-2,2-dimethyl-pyrrolidine-1-carboxylate (2.25 g, 58%). ESI-MS m/z calc. 389.3042, found 390.7 (M+1)⁺; Retention time: 3.1 minutes (LC method B).

### Step 6: tert-Butyl (4S)-4-[3-(5-tert-butyl-2-pyridyl)-3-[(6-sulfamoyl-2-pyridyl)amino]propyl]-2,2-dimethyl-pyrrolidine-1-carboxylate

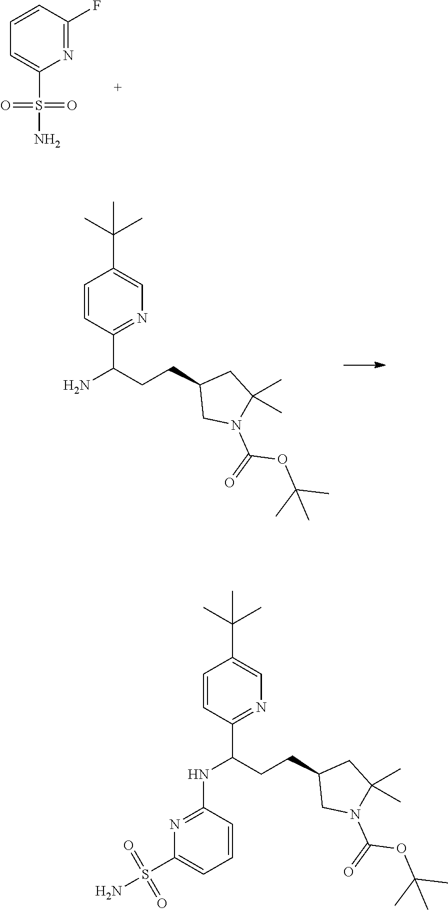 [SMILES: NS(=O)(=O)c1cccc(F)n1 ; CC(C)(C)OC(=O)N1C[C@@H](CCC(N)c2ccc(C(C)(C)C)cn2)CC1(C)C ; CC(C)(C)OC(=O)N1C[C@@H](CCC(Nc2cccc(S(N)(=O)=O)n2)c2ccc(C(C)(C)C)cn2)CC1(C)C]

To a mixture of tert-butyl (4S)-4-[3-amino-3-(5-tert-butyl-2-pyridyl)propyl]-2,2-dimethyl-pyrrolidine-1-carboxylate (2.25 g, 5.1980 mmol) and 6-fluoropyridine-2-sulfonamide (1.9 g, 10.246 mmol) in DMSO (5 mL) was added DIEA (2.2260 g, 3 mL, 17.223 mmol). The mixture was stirred at 115° C. for 18 h. The mixture was cooled and diluted with DMSO (˜10 mL). This crude sample (˜20 mL in DMSO/DIEA) was purified by prep.-HPLC (column: Varian C₁₈ 10 μm 5×30 cm; flow rate: 60 mL/min.; mobile phase A: water+0.1% TFA; mobile phase B: acetonitrile+0.1% TFA; method:0-45% B in 60 minutes). The pure fractions were combined and basified by adding saturated sodium bicarbonate and then removed acetonitrile. The cloudy water phase was extracted with ethyl acetate (2×200 mL). The organic layer was washed with brine (250 mL), dried over sodium sulfate and concentrated) to get a pale color solid (2.05 g). The solid sample was redissolved in DCM and loaded to silica gel column to be purified a second time using 0-80% ethyl acetate in hexanes to afford tert-butyl (4S)-4-[3-(5-tert-butyl-2-pyridyl)-3-[(6-sulfamoyl-2-pyridyl)amino]propyl]-2,2-dimethyl-pyrrolidine-1-carboxylate (1.94 g, 66%) as an off-white powder. ¹H NMR (500 MHz, DMSO-d₆) δ 8.58 (d, J=2.5 Hz, 1H), 7.73 (dd, J=8.2, 2.5 Hz, 1H), 7.52 (t, J=7.8 Hz, 1H), 7.47-7.37 (m, 2H), 7.06 (s, 2H), 6.96 (d, J=7.2 Hz, 1H), 6.70 (d, J=8.4 Hz, 1H), 3.53 (q, J=9.3, 8.9 Hz, 1H), 2.75 (dq, J 20.6, 10.3 Hz, 1H), 2.05 (d, J=13.4 Hz, 1H), 1.94-1.72 (m, 4H), 1.45-1.31 (m, 16H), 1.30 (s, 9H), 1.22 (s, 3H). ESI-MS m/z calc. 545.3036, found 546.4 (M+1)⁺; Retention time: 2.29 minutes (LC method A).

### Step 7: tert-Butyl (4S)-4-[3-[[6-[(6-tert-butyl-2-fluoro-pyridine-3-carbonyl)sulfamoyl]-2-pyridyl]amino]-3-(5-tert-butyl-2-pyridyl)propyl]-2,2-dimethyl-pyrrolidine-1-carboxylate

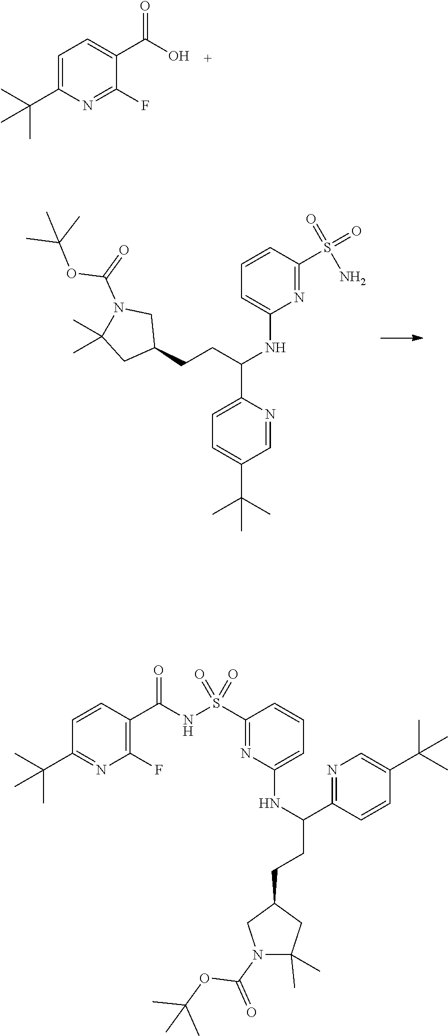 [SMILES: CC(C)(C)c1ccc(C(=O)O)c(F)n1 ; CC(C)(C)OC(=O)N1C[C@@H](CCC(Nc2cccc(S(N)(=O)=O)n2)c2ccc(C(C)(C)C)cn2)CC1(C)C ; CC(C)(C)OC(=O)N1C[C@@H](CCC(Nc2cccc(S(=O)(=O)NC(=O)c3ccc(C(C)(C)C)nc3F)n2)c2ccc(C(C)(C)C)cn2)CC1(C)C]

To a solution of 6-tert-butyl-2-fluoro-pyridine-3-carboxylic acid (230 mg, 1.166 mmol) in THF (7 mL) was added CDI (195 mg, 1.203 mmol) and the mixture was stirred at rt for 20 h. Then tert-butyl (4S)-4-[3-(5-tert-butyl-2-pyridyl)-3-[(6-sulfamoyl-2-pyridyl)amino]propyl]-2,2-dimethyl-pyrrolidine-1-carboxylate (350 mg, 0.6413 mmol) was added followed by DBU (500 μL, 3.343 mmol) and the resulting mixture was stirred for 20 h at rt. The reaction was diluted with ethyl acetate and washed with a saturated aqueous sodium bicarbonate solution. The organic layer was further washed with 10% citric acid solution followed by brine. The organics were separated, dried over sodium sulfate, and evaporated. The crude material was then purified by reverse-phase preparative chromatography utilizing a C₁₈ column and a 1-99% gradient over 15 min of acetonitrile in water (+5 mM HCl) then pumped on high vac for 3 h to afford a diastereomeric pair of tert-butyl(4S)-4-[3-[[6-[(6-tert-butyl-2-fluoro-pyridine-3-carbonyl)sulfamoyl]-2-pyridyl]amino]-3-(5-tert-butyl-2-pyridyl)propyl]-2,2-dimethyl-pyrrolidine-1-carboxylate as a white solid (380 mg, 82%). ESI-MS m/z calc. 724.37823, found 725.2 (M+1)⁺; Retention time: 1.88 minutes (LC method A).

### Step 8: (14S)-8-tert-Butyl-17-(5-tert-butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 187 (diastereomer 1), and (14S)-8-tert-butyl-17-(5-tert-butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 186 (diastereomer 2)

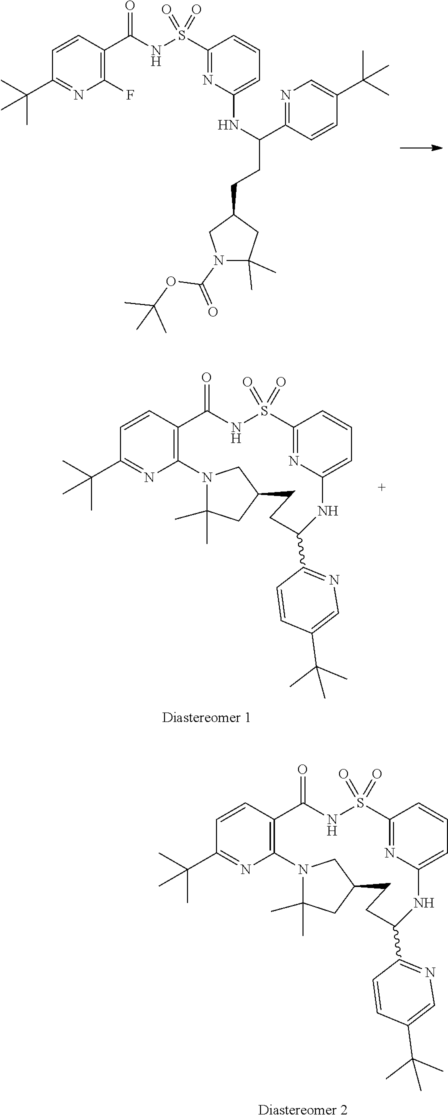

Stage 1: tert-Butyl (4S)-4-[3-[[6-[(6-tert-butyl-2-fluoro-pyridine-3-carbonyl)sulfamoyl]-2-pyridyl]amino]-3-(5-tert-butyl-2-pyridyl)propyl]-2,2-dimethyl-pyrrolidine-1-carboxylate (380 mg, 0.5242 mmol) was dissolved in DCM (9 mL) and to the mixture was added TFA (1.5 mL, 19.47 mmol) and stirred at room temperature. After 30 min, the reaction was complete. The mixture was evaporated to dryness, then diluted with diethyl ether (30 mL×2), and reconcentrated. The material was then placed on the high vacuum pump for 2 h to afford the intermediate 6-tert-butyl-N-[[6-[[1-(5-tert-butyl-2-pyridyl)-3-[(3S)-5,5-dimethylpyrrolidin-3-yl]propyl]amino]-2-pyridyl]sulfonyl]-2-fluoro-pyridine-3-carboxamide (trifluoroacetate salt) as an off-white solid.

Stage 2: Combined material from Step 1 and K₂CO₃ (750 mg, 5.427 mmol), 3 Å molecular sieves and NMP (9 mL) in a vial, purged with nitrogen, capped, heated to 155° C. and stirred for 20 h. The mixture was diluted with ethyl acetate and water. The organic layer was extracted (2×) and was further washed with 10% citric acid solution followed by brine. The organics were separated, dried over sodium sulfate, evaporated to a light brown oil. This residue was purified by reverse-phase preparative chromatography utilizing a C₁₈ column and a 15-75% gradient over 15 min of acetonitrile in water (+5 mM HCl) to afford two products:

First to elute, diastereomer 1, white solid: (14S)-8-tert-butyl-17-(5-tert-Butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (119.5 mg, 75%). ¹H NMR (400 MHz, DMSO-d₆) δ 12.52 (s, 1H), 8.71-8.54 (m, 2H), 8.19 (d, J=8.6 Hz, 2H), 7.84-7.62 (m, 2H), 7.45 (d, J=7.9 Hz, 1H), 7.36 (d, J 7.3 Hz, 1H), 6.97 (d, J=8.4 Hz, 1H), 6.62 (d, J=7.9 Hz, 1H), 5.18 (s, 1H), 4.31-4.15 (m, 1H), 3.23 (s, 1H), 2.75-2.61 (m, 1H), 2.31 (dd, J=11.6, 5.5 Hz, 1H), 2.20-2.04 (m, 1H), 1.86 (dd, J=11.7, 5.6 Hz, 1H), 1.54 (s, 6H), 1.35 (s, 9H), 1.27 (s, 9H), 0.85-0.81 (m, 2H). ESI-MS m/z calc. 604.3196, found 605.2 (M+1)⁺; Retention time: 1.73 minutes (LC method A).

Second to elute, diastereomer 2, white solid: (14S)-8-tert-butyl-17-(5-tert-butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (102.7 mg, 64%). ¹H NMR (400 MHz, DMSO-d₆) δ 12.43 (s, 1H), 8.66 (d, J=2.4 Hz, 1H), 8.35 (s, 1H), 8.05-7.73 (m, 2H), 7.73-7.68 (m, 1H), 7.64 (d, J=8.0 Hz, 1H), 7.18 (d, J=7.1 Hz, 1H), 6.95 (d, J=8.5 Hz, 1H), 6.66 (d, J=8.0 Hz, 1H), 5.43 (s, 1H), 3.30 (t, J=8.3 Hz, 1H), 2.71 (d, J=10.3 Hz, 1H), 2.05-1.92 (m, 2H), 1.85 (dd, J=11.7, 5.1 Hz, 1H), 1.77 (d, J=14.2 Hz, 1H), 1.67 (s, 3H), 1.57 (t, J=12.4 Hz, 1H), 1.51 (s, 3H), 1.33 (s, 9H), 1.28 (s, 9H), 0.89-0.75 (m, 2H). ESI-MS m/z calc. 604.3196, found 605.2 (M+1)⁺; Retention time: 1.71 minutes (LC method A).

### Example 50: Preparation of (14S)-8-tert-Butyl-17-(6-tert-butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 179 (diastereomer 1) and Compound 178 (diastereomer 2)

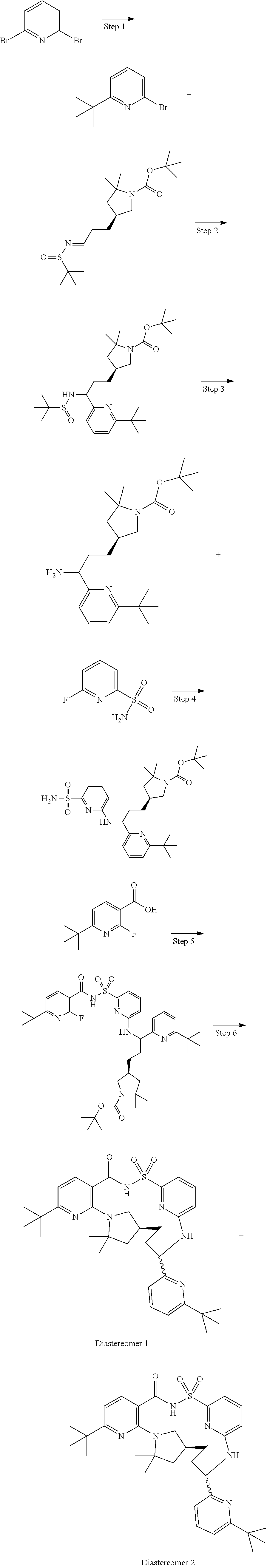

### Step 1: 2-Bromo-6-tert-butyl-pyridine

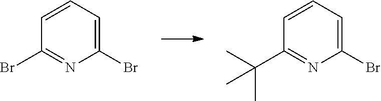

2,6-Dibromopyridine (12.3 g, 50.884 mmol) was dissolved in THF (100 mL). Cut (2.48 g, 13.022 mmol) was added. The mixture was stirred in a dry ice acetone bath below −70° C. under a nitrogen balloon. tert-Butylmagnesium chloride (78.2 mL of 1 M, 78.200 mmol) was then added slowly over 15 min. The reaction mixture was allowed to warm up on its own to rt and stirred at rt for 15 h. Saturated aqueous NH₄Cl (50 mL) was added. The mixture was concentrated to remove most of the THF and then extracted with DCM (50 mL×3). The combined organics were dried over anhydrous MgSO₄, filtered and concentrated. The residue was purified by silica gel chromatography (120 g column), using 0-20% EtOAc in hexanes, to afford 2-bromo-6-tert-butyl-pyridine (8.2 g, 72%) as a white semisolid. ESI-MS m/z calc. 213.0153, found 214.3 (M+1)⁺; Retention time: 3.74 minutes (LC method B).

### Step 2: tert-Butyl (4S)-4-[3-(6-tert-butyl-2-pyridyl)-3-(tert-butylsulfinylamino)propyl]-2,2-dimethyl-pyrrolidine-1-carboxylate

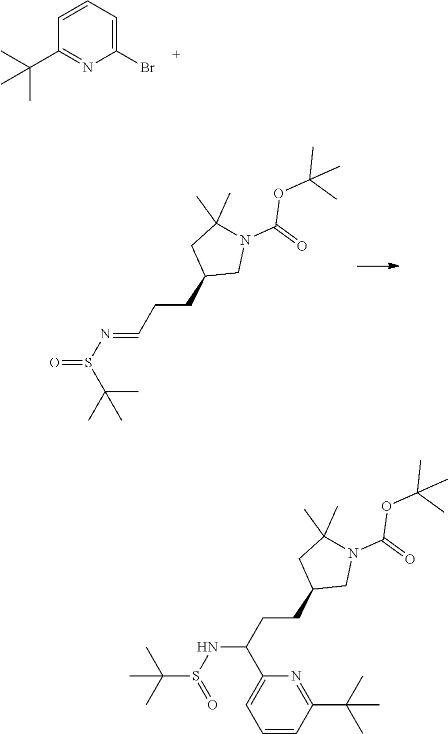

2-Bromo-6-tert-butyl-pyridine (8.2 g, 36.385 mmol) was dissolved in THF (120 mL) and stirred under nitrogen balloon in a dry ice acetone bath for 15 min. n-BuLi (15.2 mL of 2.5 M in hexanes, 38.00 mmol) was added in a quick dropwise fashion within 5 min. The mixture was stirred below −70° C. for 30 min. tert-butyl (4S)-4-[(3E)-3-tert-butylsulfinyliminopropyl]-2,2-dimethyl-pyrrolidine-1-carboxylate (6.8 g, 18.966 mmol) in THF (10 mL plus 2 mL rinse) was added. The mixture was stirred at the same temperature for another 15 min. NH₄Cl (20 mL, saturated aqueous) was added. The mixture was removed from the cooling bath. Water (50 mL) and EtOAc (100 mL) were added and the mixture was stirred in a warm water bath until two homogeneous layers were formed. The layers were separated. The organic layer was dried over anhydrous Na₂SO₄, filtered and concentrated. The residue was purified by silica gel chromatography 120 g column), using 0-40% EtOAc in hexanes to afford crude product tert-butyl (4S)-4-[3-(6-tert-butyl-2-pyridyl)-3-(tert-butylsulfinylamino)propyl]-2,2-dimethyl-pyrrolidine-1-carboxylate (8 g, 77%). ESI-MS m/z calc. 493.3338, found 494.7 (M+1)+; Retention time: 3.46 minutes (LC method B).

### Step 3: tert-Butyl (4S)-4-[3-amino-3-(6-tert-butyl-2-pyridyl)propyl]-2,2-dimethyl-pyrrolidine-1-carboxylate

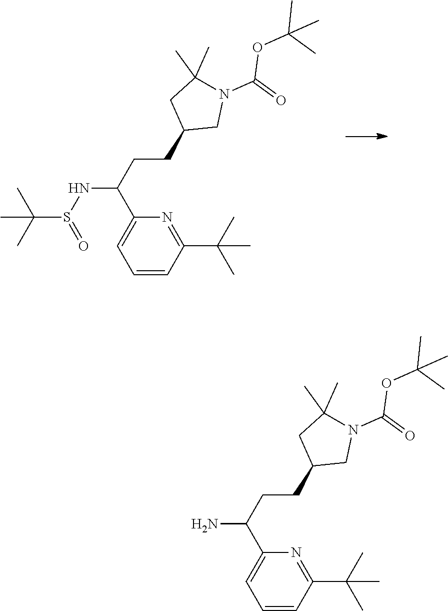 [SMILES: CC(C)(C)OC(=O)N1C[C@@H](CCC(NS(=O)C(C)(C)C)c2cccc(C(C)(C)C)n2)CC1(C)C ; CC(C)(C)OC(=O)N1C[C@@H](CCC(N)c2cccc(C(C)(C)C)n2)CC1(C)C]

tert-Butyl (4S)-4-[3-(6-tert-butyl-2-pyridyl)-3-(tert-butylsulfinylamino)propyl]-2,2-dimethyl-pyrrolidine-1-carboxylate (8 g, 14.582 mmol) was dissolved in a solvent mixture of THF (50 mL) and water (10 mL). Molecular iodine (1.11 g, 0.2252 mL, 4.3734 mmol) was added in one portion. The mixture was then heated in a 50° C. oil bath for 3 h. It was then cooled to rt. Na₂S₂O₃ (10 g in 30 mL saturated aqueous sodium bicarbonate) was added, followed by EtOAc (50 mL). The layers were separated and the organic layer was washed with brine, dried over anhydrous Na₂SO₄, filtered and concentrated. The residue was purified by silica gel chromatography (80 g column), using 0-5% MeOH in DCM to afford crude tert-butyl (4S)-4-[3-amino-3-(6-tert-butyl-2-pyridyl)propyl]-2,2-dimethyl-pyrrolidine-1-carboxylate as a white foam (4.88 g, 82%). ESI-MS m/z calc. 389.3042, found 390.7 (M+1)⁺; Retention time: 3.3 minutes (LC method B).

### Step 4: tert-Butyl (4S)-4-[3-(6-tert-butyl-2-pyridyl)-3-[(6-sulfamoyl-2-pyridyl)amino]propyl]-2,2-dimethyl-pyrrolidine-1-carboxylate

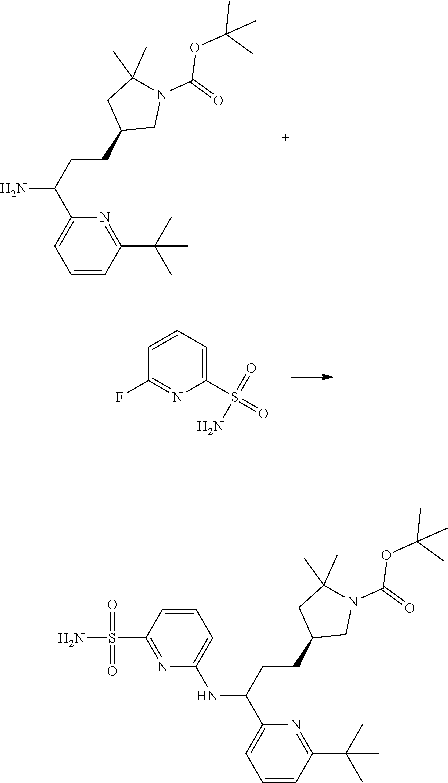 [SMILES: CC(C)(C)OC(=O)N1C[C@@H](CCC(N)c2cccc(C(C)(C)C)n2)CC1(C)C ; NS(=O)(=O)c1cccc(F)n1 ; CC(C)(C)OC(=O)N1C[C@@H](CCC(Nc2cccc(S(N)(=O)=O)n2)c2cccc(C(C)(C)C)n2)CC1(C)C]

tert-Butyl (4S)-4-[3-amino-3-(6-tert-butyl-2-pyridyl)propyl]-2,2-dimethyl-pyrrolidine-1-carboxylate (4.8 g, 11.705 mmol) was dissolved in DMSO (5 mL). 6-Fluoropyridine-2-sulfonamide (2.6 g, 14.759 mmol) was added, followed by Na₂CO₃ (4 g, 37.740 mmol). The mixture was well stirred under a nitrogen balloon and placed in a 110° C. oil bath. After 20 h, the mixture was cooled to rt, diluted with water (50 mL) and EtOAc (50 mL). The layers were separated and the organic layer was dried over anhydrous MgSO₄, filtered and concentrated. The residue was purified by silica gel chromatography (80 g column), using 0-50% EtOAc in hexanes to afford tert-butyl (4S)-4-[3-(6-tert-butyl-2-pyridyl)-3-[(6-sulfamoyl-2-pyridyl)amino]propyl]-2,2-dimethyl-pyrrolidine-1-carboxylate as a white foam (2.7539 g, 42%). ¹H NMR (500 MHz, DMSO-d₆) δ 7.64 (td, J=7.8, 1.4 Hz, 1H), 7.54 (dd, J=8.6, 7.2 Hz, 1H), 7.32 (t, J=7.9 Hz, 1H), 7.29-7.22 (m, 2H), 7.07 (s, 2H), 6.98 (d, J=7.2 Hz, 1H), 6.74 (d, J=8.4 Hz, 1H), 5.18 (s, 1H), 3.52 (q, J=11.1 Hz, 1H), 2.81-2.65 (m, 1H), 2.07 (s, 1H), 1.97 (s, 1H), 1.91-1.77 (m, 2H), 1.43-1.35 (m, 11H), 1.33 (s, 14H), 1.22 (d, J=1.3 Hz, 3H). ESI-MS m/z calc. 545.3036, found 546.4 (M+1)⁺; Retention time: 2.27 minutes (LC method H).

### Step 5: tert-Butyl (4S)-4-[3-[[6-[(6-tert-butyl-2-fluoro-pyridine-3-carbonyl)sulfamoyl]-2-pyridyl]amino]-3-(6-tert-butyl-2-pyridyl)propyl]-2,2-dimethyl-pyrrolidine-1-carboxylate

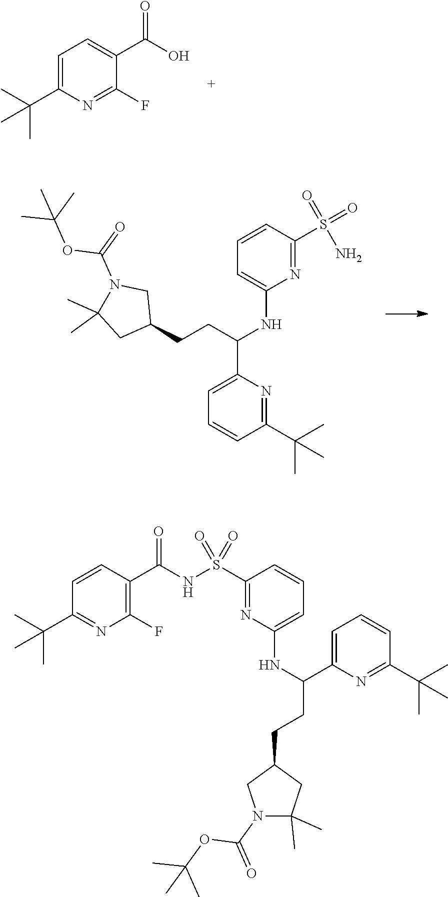 [SMILES: CC(C)(C)c1ccc(C(=O)O)c(F)n1 ; CC(C)(C)OC(=O)N1C[C@@H](CCC(Nc2cccc(S(N)(=O)=O)n2)c2cccc(C(C)(C)C)n2)CC1(C)C ; CC(C)(C)OC(=O)N1C[C@@H](CCC(Nc2cccc(S(=O)(=O)NC(=O)c3ccc(C(C)(C)C)nc3F)n2)c2cccc(C(C)(C)C)n2)CC1(C)C]

To a solution of 6-tert-butyl-2-fluoro-pyridine-3-carboxylic acid (165 mg, 0.8367 mmol) in THF (5.0 mL) was added CDI (140 mg, 0.8634 mmol) and the mixture was stirred at rt for 20 h. Then tert-butyl (4S)-4-[3-(6-tert-butyl-2-pyridyl)-3-[(6-sulfamoyl-2-pyridyl)amino]propyl]-2,2-dimethyl-pyrrolidine-1-carboxylate (250 mg, 0.4581 mmol) was added followed by DBU (375 μL, 2.508 mmol) and the resulting mixture was stirred for 4 h at rt. The reaction was diluted with ethyl acetate and washed with a saturated aqueous sodium bicarbonate solution. The organic layer was further washed with 10% citric acid solution followed by brine. The organics were separated, dried over sodium sulfate, and evaporated. The crude material was then purified by reverse-phase preparative chromatography utilizing a C₁₈ column and a 1-99% gradient over 15 min of acetonitrile in water (+5 mM HCl) then pumped on high vac for 3 h to afford the diastereomeric pair of tert-butyl (4S)-4-[3-[[6-[(6-tert-butyl-2-fluoro-pyridine-3-carbonyl)sulfamoyl]-2-pyridyl]amino]-3-(6-tert-butyl-2-pyridyl)propyl]-2,2-dimethyl-pyrrolidine-1-carboxylate as a white solid (190 mg, 57%). ESI-MS m/z calc. 724.37823, found 725.2 (M+1)⁺; Retention time: 2.16 minutes (LC method A).

### Step 6: (14S)-8-tert-Butyl-17-(6-tert-butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 179 (diastereomer 1) and (14S)-8-tert-butyl-17-(6-tert-butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 178 (diastereomer 2)

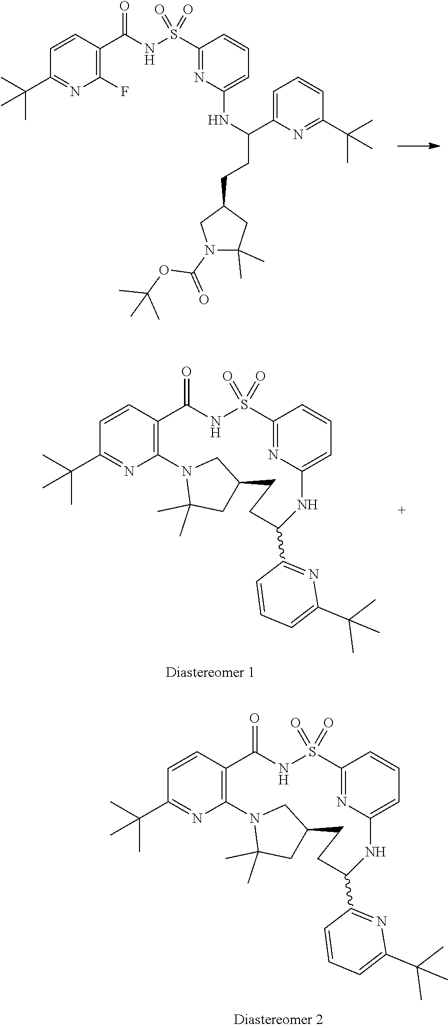

Stage 1:

tert-Butyl (4S)-4-[3-[[6-[(6-tert-butyl-2-fluoro-pyridine-3-carbonyl)sulfamoyl]-2-pyridyl]amino]-3-(6-tert-butyl-2-pyridyl)propyl]-2,2-dimethyl-pyrrolidine-1-carboxylate (190 mg, 0.2621 mmol) was dissolved in DCM (5.5 mL) and to the mixture was added TFA (750 μL, 9.735 mmol) and stirred at room temperature. After 30 min, the mixture was evaporated to dryness, then diluted with diethyl ether (30 mL×2), and reconcentrated. The material was then placed on the high vacuum pump for 2 h to afford the intermediate 6-tert-butyl-N-[[6-[[1-(6-tert-butyl-2-pyridyl)-3-[(3S)-5,5-dimethylpyrrolidin-3-yl]propyl]amino]-2-pyridyl]sulfonyl]-2-fluoro-pyridine-3-carboxamide (trifluoroacetate salt) ESI-MS m/z calc. 624.3258, found 625.2 (M+1)⁺; Retention time: 1.38 minutes as an off-white solid (LC method A).

Stage 2:

Combined material from Step 1 and K₂CO₃ (375 mg, 2.713 mmol), 3 Å molecular sieves and NMP (4 mL) in a vial, purged with nitrogen, capped, heated to 150° C. and stirred for 20 h. The mixture was diluted with ethyl acetate and water. The organic layer was extracted (2×) and was further washed with 10% citric acid solution followed by brine. The organics were separated, dried over sodium sulfate, evaporated to a light brown oil. This residue was purified by reverse-phase preparative chromatography utilizing a C₁₈ column and a 30-99% gradient over 15 min of acetonitrile in water (+5 mM HCl) to afford two isomeric products:

Diastereomer 1, more polar, white solid: (14S)-8-tert-Butyl-17-(6-tert-butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (34.2 mg, 43%). ¹H NMR (400 MHz, DMSO-d₆) δ 12.47 (s, 1H), 7.62 (dt, J=22.7, 7.3 Hz, 4H), 7.25 (s, 2H), 7.11 (d, J=7.2 Hz, 1H), 6.91 (d, J=8.5 Hz, 1H), 6.65 (d, J=8.0 Hz, 1H), 5.22 (s, 1H), 3.17 (s, 1H), 2.75 (t, J=10.4 Hz, 1H), 2.18 (d, J=16.5 Hz, 2H), 1.86 (dd, J=11.7, 5.2 Hz, 2H), 1.75 (d, J=14.6 Hz, 1H), 1.65 (s, 3H), 1.58 (t, J=12.3 Hz, 1H), 1.51 (s, 3H), 1.45 (d, J=11.4 Hz, 1H), 1.30 (d, J=17.4 Hz, 18H). ESI-MS m/z calc. 604.3196, found 605.2 (M+1)⁺; Retention time: 2.02 minutes (LC method A).

Diastereromer 2, less polar, white solid: (14S)-8-tert-Butyl-17-(6-tert-butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (42.0 mg, 52%). ¹H NMR (400 MHz, DMSO-d₆) δ 12.32 (s, 1H), 7.65 (t, J=7.9 Hz, 4H), 7.42 (d, J=31.8 Hz, 2H), 7.25 (s, 1H), 6.90 (d, J=8.5 Hz, 1H), 6.62 (d, J=7.9 Hz, 1H), 5.05 (s, 1H), 3.26-3.11 (m, 1H), 3.03 (d, J=6.4 Hz, 1H), 2.41-2.18 (m, 2H), 2.18-2.07 (m, 1H), 1.93-1.83 (m, 1H), 1.75 (s, 2H), 1.56 (d, J=23.0 Hz, 6H), 1.43 (s, 1H), 1.36 (s, 9H), 1.27 (s, 9H). ESI-MS m/z calc. 604.3196, found 605.2 (M+1)⁺; Retention time: 2.07 minutes (LC method A).

### Example 51: Preparation of (14S)-8-tert-Butyl-17-(3,3-dicyclopropylpropyl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, Compound 51 (diastereomer 1) and Compound 52 (diastereomer 2)

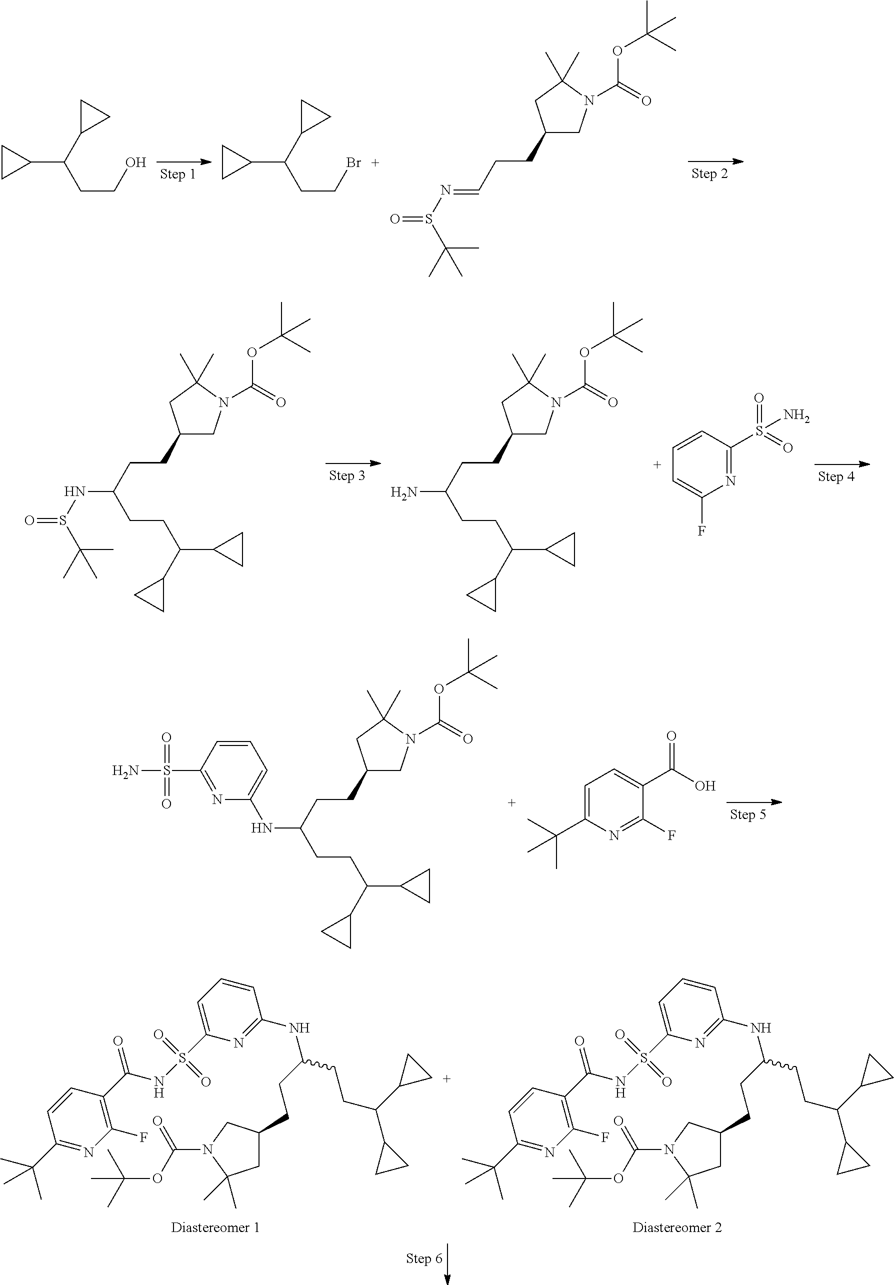

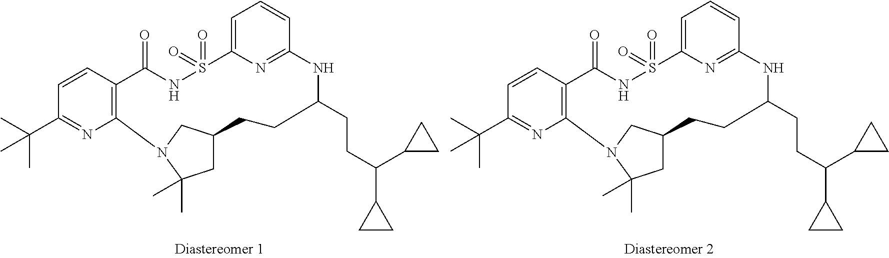

### Step 1: (3-Bromo-1-cyclopropyl-propyl)cyclopropane

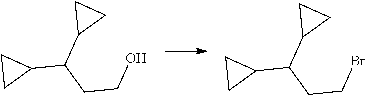 [SMILES: OCCC(C1CC1)C1CC1 ; BrCCC(C1CC1)C1CC1]

3,3-Dicyclopropylpropan-1-ol (4.1 g, 29.239 mmol) was dissolved in DCM (150 mL). Triphenylphosphine (12.3 g, 46.896 mmol) was added. The solution was stirred in ice water bath under a nitrogen balloon for 20 min. NBS (8.35 g, 46.914 mmol) was then added in portions. The mixture was stirred at the same temperature for 5 h, warmed up to rt and concentrated (150 mmHg, <28° C. bath). The residue was treated with diethyl ether (2 mL), sonicated for 1 min and diluted with pentane (20 mL). The mixture was stirred for 15 h. It was then filtered. The filtrate (combined with that of VBL00771-348) was concentrated carefully (volatile product). The residue was purified by silica gel chromatography (120 g column), using 0-10% diethyl ether in pentane to afford (3-bromo-1-cyclopropyl-propyl)cyclopropane as a clear oil (6 g, 91%). ¹H NMR (250 MHz, Chloroform-d) δ 3.79-3.54 (m, 2H), 2.29-1.89 (m, 2H), 1.24 (m, 1H), 0.79-0.11 (m, 12H).

### Step 2: tert-Butyl (4S)-4-[3-(tert-butylsulfinylamino)-6,6-dicyclopropyl-hexyl]-2,2-dimethyl-pyrrolidine-1-carboxylate

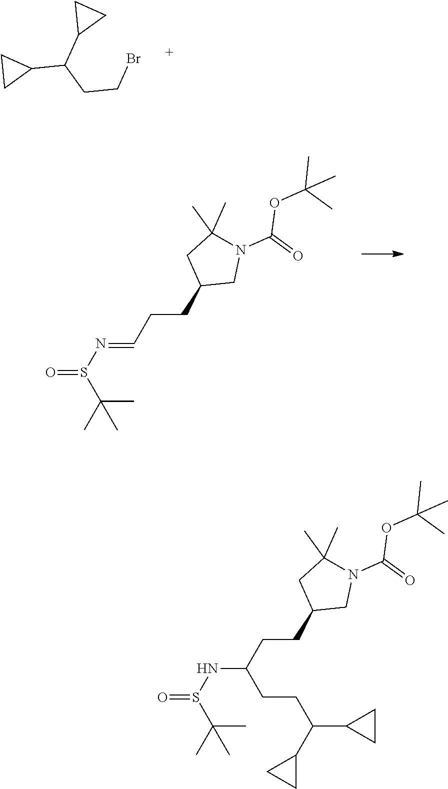 [SMILES: BrCCC(C1CC1)C1CC1 ; CC(C)(C)OC(=O)N1C[C@@H](CC/C=N/S(=O)C(C)(C)C)CC1(C)C ; CC(C)(C)OC(=O)N1C[C@@H](CCC(CCC(C2CC2)C2CC2)NS(=O)C(C)(C)C)CC1(C)C]

Mg (841 mg, 34.602 mmol) was weighted into a dry vessel. A tiny iodine crystal was added. with a nitrogen balloon on top, the flask was heated with a heating gun briefly until some iodine vapor was seen. The flask was cooled to rt. THF (60 mL) was added, followed by some dibromoethane (0.1 mL). The mixture was stirred efficiently for 5 min. The iodine color disappeared. A solution of (3-bromo-1-cyclopropyl-propyl)cyclopropane (5.7 g, 25.256 mmol) in THF (20 mL) was added in small portions via a syringe over 20 min. The reaction was slightly exothermic. The mixture was further stirred at rt for 1 h after complete addition. It was then cooled to ˜−30° C. using a dry ice acetone bath. tert-butyl (4S)-4[(3E)-3-tert-butylsulfinyliminopropyl]-2,2-dimethyl-pyrrolidine-1-carboxylate (4 g, 11.156 mmol) in THF (20 mL) was added dropwise. After stirring at this temperature for 30 min, NH₄Cl (40 mL, saturated aqueous) was added, followed by EtOAc (100 mL). The layers were separated and the organic layer was combined with a crude solution from another reaction run in similar conditions on 360 mg scale. It was washed with brine, dried over anhydrous MgSO₄, filtered and concentrated to afford crude tert-butyl (4S)-4-[(3-(tert-butylsulfinylamino)-6,6-dicyclopropyl-hexyl]-2,2-dimethyl-pyrrolidine-1-carboxylate (5.2 g, 87% adjusted yield) as pale yellow oil. ESI-MS m/z calc. 482.3542, found 483.7 (M+1)⁺; Retention time: 4.84 minutes (LC method B).

### Step 3: tert-Butyl (4S)-4-(3-amino-6,6-dicyclopropyl-hexyl)-2,2-dimethyl-pyrrolidine-1-carboxylate

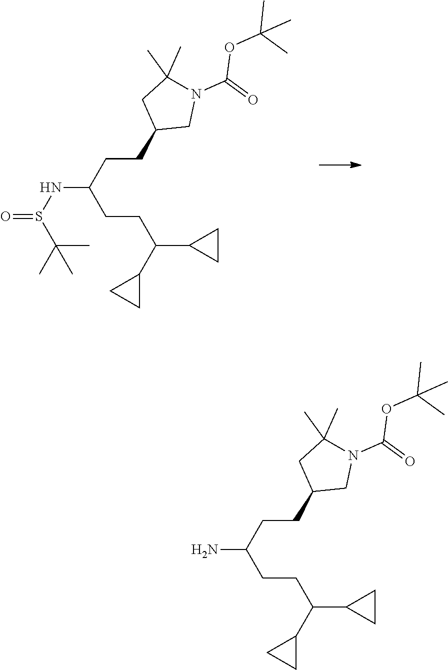 [SMILES: CC(C)(C)OC(=O)N1C[C@@H](CCC(CCC(C2CC2)C2CC2)NS(=O)C(C)(C)C)CC1(C)C ; CC(C)(C)OC(=O)N1C[C@@H](CCC(N)CCC(C2CC2)C2CC2)CC1(C)C]

tert-Butyl (4S)-4-[3-(tert-butylsulfinylamino)-6,6-dicyclopropyl-hexyl]-2,2-dimethyl-pyrrolidine-1-carboxylate (5.2 g, 9.6942 mmol) was dissolved in a solvent mixture of THF (50 mL) and Water (10 mL). Molecular iodine (493 mg, 0.1000 mL, 1.9424 mmol) was added in one portion. The mixture was heated in a 50° C. oil bath for 3 h. It was then cooled to rt and treated with Na₂S₂O₃ (2 g in 20 mL saturated aqueous sodium bicarbonate). EtOAc (50 mL) was added, followed by water (50 mL). The layers were separated and the organic layer was washed with brine, dried over anhydrous MgSO₄, filtered and concentrated. The pale yellow colored foam crude was used in the next step without further purification. tert-butyl (4S)-4-(3-amino-6,6-dicyclopropyl-hexyl)-2,2-dimethyl-pyrrolidine-1-carboxylate (5.5 g, 120%). ESI-MS m/z calc. 378.3246, found 379.8 (M+1)⁺; Retention time: 3.39 minutes (LC method B).

### Step 4: tert-Butyl (4S)-4-[6,6-dicyclopropyl-3-[(6-sulfamoyl-2-pyridyl)amino]hexyl]-2,2-dimethyl-pyrrolidine-1-carboxylate

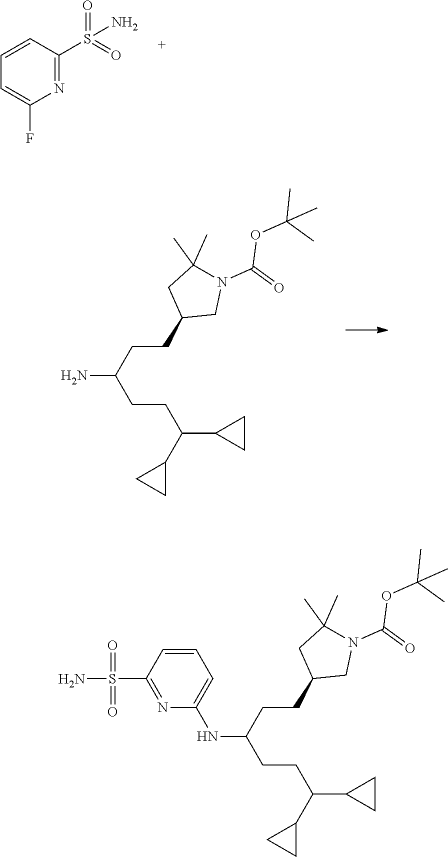 [SMILES: NS(=O)(=O)c1cccc(F)n1 ; CC(C)(C)OC(=O)N1C[C@@H](CCC(N)CCC(C2CC2)C2CC2)CC1(C)C ; CC(C)(C)OC(=O)N1C[C@@H](CCC(CCC(C2CC2)C2CC2)Nc2cccc(S(N)(=O)=O)n2)CC1(C)C]

tert-Butyl (4S)-4-(3-amino-6,6-dicyclopropyl-hexyl)-2,2-dimethyl-pyrrolidine-1-carboxylate (5.5 g, 11.622 mmol) was dissolved in DMSO (4 mL) at rt. 6-Fluoropyridine-2-sulfonamide (2.05 g, 11.637 mmol) was added, followed by Na₂CO₃ (3.8 g, 35.853 mmol). The mixture was heated in a 110° C. oil bath under a nitrogen balloon for 24 h. It was then cooled to rt and diluted with EtOAc (50 mL) and water (50 mL). The layers were separated and the organic layer was washed with more water (30 mL×2) and brine (30 mL). It was then dried over anhydrous MgSO₄, filtered and concentrated. The residue was purified by silica gel chromatography (80 g column), using 0-60% EtOAc/hexanes to afford tert-butyl (4S)-4-[6,6-dicyclopropyl-3-[(6-sulfamoyl-2-pyridyl)amino]hexyl]-2,2-dimethyl-pyrrolidine-1-carboxylate (4.52 g, 68%) as a white foam. ¹H NMR (500 MHz, DMSO-d₆) δ 7.49 (t, J=7.8 Hz, 1H), 7.03 (s, 2H), 6.92 (d, J=7.1 Hz, 1H), 6.76 (d, J=8.6 Hz, 1H), 6.61 (d, J=8.5 Hz, 1H), 3.53 (tt, J=12.9, 5.3 Hz, 1H), 2.75 (ddd, J=17.4, 8.7, 5.0 Hz, 1H), 2.11-2.02 (m, 1H), 1.87 (ddd, J=18.2, 12.6, 5.2 Hz, 1H), 1.66 (d, J=13.3 Hz, 1H), 1.60-1.43 (m, 5H), 1.43-1.28 (m, 18H), 1.23 (s, 3H), 0.57 (qt, J=9.2, 4.5 Hz, 2H), 0.33 (ddt, J=16.5, 8.0, 4.2 Hz, 4H), 0.13 (d, J=8.3 Hz, 2H), 0.10-−0.00 (m, 2H), −0.07 (dd, J=8.8, 3.8 Hz, 1H). ESI-MS m/z calc. 534.324, found 535.3 (M+1)⁺; Retention time: 3.27 minutes (LC method H).

### Step 5: tert-Butyl (4S)-4-[3-[[6-[(6-tert-butyl-2-fluoro-pyridine-3-carbonyl)sulfamoyl]-2-pyridyl]amino]-6,6-dicyclopropyl-hexyl]-2,2-dimethyl-pyrrolidine-1-carboxylate, diastereomer 1 and tert-butyl (4S)-4-[3-[[6-[(6-tert-butyl-2-fluoro-pyridine-3-carbonyl)sulfamoyl]-2-pyridyl]amino]-6,6-dicyclopropyl-hexyl]-2,2-dimethyl-pyrrolidine-1-carboxylate, diastereomer 2

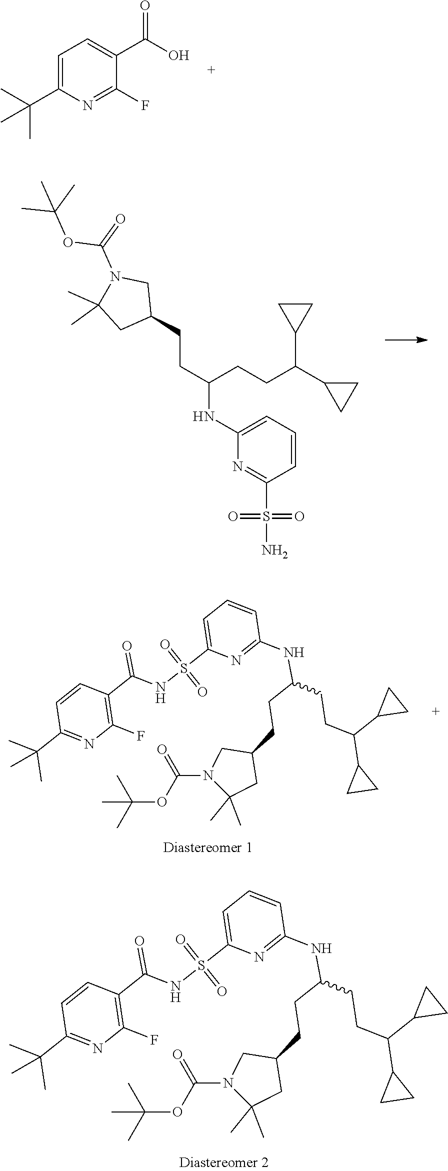

To a solution of 6-tert-butyl-2-fluoro-pyridine-3-carboxylic acid (187 mg, 0.9482 mmol) in THF (2 mL) was added CDI (156 mg, 0.9621 mmol) and the mixture was stirred at rt for 16 h then tert-butyl (4S)-4-[6,6-dicyclopropyl-3-[(6-sulfamoyl-2-pyridyl)amino]hexyl]-2,2-dimethyl-pyrrolidine-1-carboxylate (400 mg, 0.7480 mmol) followed by DBU (350 μL, 2.340 mmol) was added and the resulting mixture was stirred for 2 h at room temperature. Reaction was quenched with a 1:1 mixture of saturated ammonium chloride and brine solutions, and then extracted with ethyl acetate. The combined organic layers were washed with brine, dried over sodium sulfate, filtered and evaporated. The resultant residue was purified by a reverse phase chromatography using a dual gradient run of 50-100% mobile phase B over 20.0 minutes (mobile phase A=H₂O (5 mM HCl), mobile phase B=CH₃CN) to afford two isomers:

First eluting diastereomer 1: tert-Butyl (4S)-4-[3-[[6-[(6-tert-butyl-2-fluoro-pyridine-3-carbonyl)sulfamoyl]-2-pyridyl]amino]-6,6-dicyclopropyl-hexyl]-2,2-dimethyl-pyrrolidine-1-carboxylate (112 mg, 42%). ESI-MS m/z calc. 713.3986, found 714.5 (M+1)⁺; Retention time: 1.84 minutes (LC method M).

Second eluting diastereomer 2: tert-Butyl (4S)-4-[3-[[6-[(6-tert-butyl-2-fluoro-pyridine-3-carbonyl)sulfamoyl]-2-pyridyl]amino]-6,6-dicyclopropyl-hexyl]-2,2-dimethyl-pyrrolidine-1-carboxylate (124 mg, 46%). ESI-MS m/z calc. 713.3986, found 714.5 (M+1)⁺; Retention time: 1.89 minutes (LC method M).

### Step 6: (14S)-8-tert-Butyl-17-(3,3-dicyclopropylpropyl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione, Compound 51 (diastereomer 1)

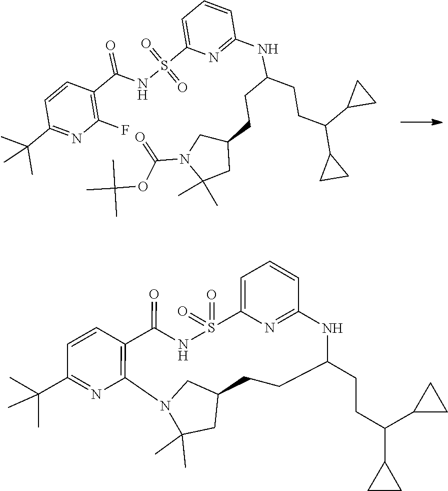 [SMILES: CC(C)(C)OC(=O)N1C[C@@H](CCC(CCC(C2CC2)C2CC2)Nc2cccc(S(=O)(=O)NC(=O)c3ccc(C(C)(C)C)nc3F)n2)CC1(C)C ; CC(C)(C)c1ccc2c(n1)N1C[C@@H](CCC(CCC(C3CC3)C3CC3)Nc3cccc(n3)S(=O)(=O)NC2=O)CC1(C)C]

A solution of tert-butyl (4S)-4-[3-[[6-[(6-tert-butyl-2-fluoro-pyridine-3-carbonyl)sulfamoyl]-2-pyridyl]amino]-6,6-dicyclopropyl-hexyl]-2,2-dimethyl-pyrrolidine-1-carboxylate (110 mg, 0.1541 mmol) (diastereomer 1) in 1:4 pre mixed solution of TFA (125 μL, 1.622 mmol) and DCM (500 μL) was stirred at rt for 2 h. The solvent was removed and dried on high vac pump. The resultant residue was dissolved in DMSO (2 mL) and K₂CO₃ (347 mg, 2.511 mmol) was added and the microwave vial was capped and the mixture was heated at 140° C. for 16 h. Cooled to rt and the mixture was filtered through Whatman filter disc (puradisc 25 TF) and filtrate was purified by a reverse phase HPLC-MS method using a dual gradient run of 30-99% mobile phase B over 15.0 minutes (mobile phase A=H₂O (5 mM HCl), mobile phase B=CH₃CN) to afford as a white solid, (14S)-8-tert-butyl-17-(3,3-dicyclopropylpropyl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (hydrochloride salt) (45.1 mg, 46%). ¹H NMR (400 MHz, DMSO-d₆) δ 12.38 (s, 1H), 7.55 (t, J=7.9 Hz, 1H), 7.39 (d, J=7.9 Hz, 1H), 7.16 (d, J=7.3 Hz, 2H), 6.73 (d, J=8.5 Hz, 1H), 6.60 (d, J=7.9 Hz, 1H), 3.28 (s, 1H), 3.16 (d, J=9.5 Hz, 1H), 2.90 (s, 1H), 2.23 (s, 2H), 1.90 (s, 1H), 1.81 (dd, J=12.0, 5.6 Hz, 1H), 1.66 (d, J=13.9 Hz, 2H), 1.56 (s, 3H), 1.50 (d, J=16.1 Hz, 7H), 1.26 (s, 9H), 1.14 (q, J=10.3, 9.2 Hz, 1H), 0.53 (dp, J=13.3, 6.2 Hz, 2H), 0.33 (dd, J=8.7, 4.1 Hz, 3H), 0.25 (q, J=4.3 Hz, 1H), 0.12 (dt, J=8.6, 4.5 Hz, 2H), 0.04 (dt, J=13.9, 6.6 Hz, 2H), −0.07 (p, J=4.8 Hz, 1H). ESI-MS m/z calc. 593.33997, found 594.5 (M+1)⁺; Retention time: 1.78 minutes (LC method M).

The compound in the following tables was prepared in a manner analogous to that described above using tert-butyl (4S)-4-[3-[[6-[(6-tert-butyl-2-fluoro-pyridine-3-carbonyl)sulfamoyl]-2-pyridyl]amino]-6,6-dicyclopropyl-hexyl]-2,2-dimethyl-pyrrolidine-1-carboxylate (110 mg, 0.1541 mmol) (diastereomer 2) as a starting material:

| | | LCMS | | | |
|---|---|---|---|---|---|
| Compound | | Retention | Exact | | LCMS |
| Number | Structure | Time (min) | Mass | M + 1 | Method |
| Compound 52, hydrochloride salt | 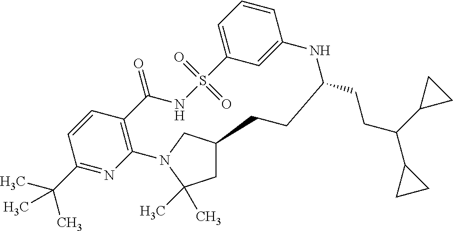 [SMILES: CC(C)(C)c1ccc2c(n1)N1C[C@@H](CC[C@@H](CCC(C3CC3)C3CC3)Nc3cccc(c3)S(=O)(=O)NC2=O)CC1(C)C] | 1.68 | 593.34 | 594.5 | LC method M |

| Compound | |
|---|---|
| Number | NMR |
| Compound | ¹H NMR (400 MHz, DMSO-d₆) δ 12.42 (s, 1H), |
| 52 | 7.64-7.48 (m, 2H), 7.02 (dd, J = 7.1, 1.9 Hz, 1H), |
| | 6.90 (d, J = 9.0 Hz, 1H), 6.69 (dd, J = 8.5, 1.8 Hz, |
| | 1H), 6.63 (dd, J = 8.0, 2.0 Hz, 1H), 4.09 (d, J = 12.9 |
| | Hz, 1H), 3.04 (t, J = 8.6 Hz, 1H), 2.65 (t, J = 10.5 |
| | Hz, 1H), 2.15-2.01 (m, 1H), 1.79 (dd, J = 12.3, 5.1 |
| | Hz, 1H), 1.66-1.55 (m, 6H), 1.54-1.37 (m, 8H), |
| | 1.27 (d, J = 2.0 Hz, 10H), 0.54 (q, J = 8.8, 7.3 Hz, |
| | 2H), 0.39-0.22 (m, 4H), 0.16-0.07 (m, 2H), 0.04 |
| | (d, J = 6.7 Hz, 1H), 0.00-−0.10 (m, 2H). |

### Example 52: Preparation of (14S,17R)-8-(3,3-dimethylbutyl)-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 105

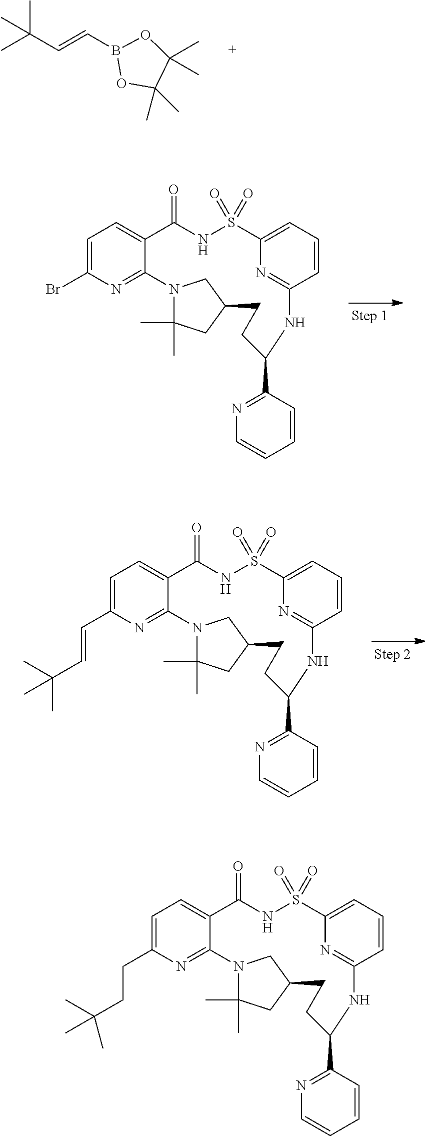 [SMILES: CC(C)(C)/C=C/B1OC(C)(C)C(C)(C)O1 ; CC1(C)C[C@@H]2CC[C@H](c3ccccn3)Nc3cccc(n3)S(=O)(=O)NC(=O)c3ccc(Br)nc3N1C2 ; CC(C)(C)/C=C/c1ccc2c(n1)N1C[C@@H](CC[C@H](c3ccccn3)Nc3cccc(n3)S(=O)(=O)NC2=O)CC1(C)C ; CC(C)(C)CCc1ccc2c(n1)N1C[C@@H](CC[C@H](c3ccccn3)Nc3cccc(n3)S(=O)(=O)NC2=O)CC1(C)C]

### Step 1: (14S,17R)-8-[(1E)-3,3-dimethylbut-1-en-1-yl]-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 106

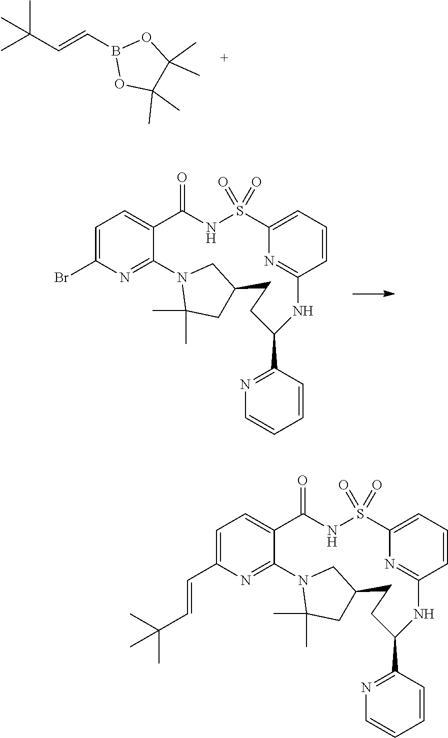 [SMILES: CC(C)(C)/C=C/B1OC(C)(C)C(C)(C)O1 ; CC1(C)C[C@@H]2CC[C@H](c3ccccn3)Nc3cccc(n3)S(=O)(=O)NC(=O)c3ccc(Br)nc3N1C2 ; CC(C)(C)/C=C/c1ccc2c(n1)N1C[C@@H](CC[C@H](c3ccccn3)Nc3cccc(n3)S(=O)(=O)NC2=O)CC1(C)C]

A mixture of (14S,17R)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (40 mg, 0.070 mmol), 2-[(E)-3,3-dimethylbut-1-enyl]-4,4,5,5-tetramethyl-1,3,2-dioxaborolane (59 mg, 0.281 mmol), Pd(dppf)Cl₂ (5 mg, 0.0068 mmol), acetonitrile (700 μL) and NaHCO₃(420 μL of 1 M, 0.420 mmol) was degassed by bubbling nitrogen for 1 min then heated to 100° C. for 3 h. Then the mixture was diluted with 2 mL water, 2 mL of 5:1 DCM/MeOH, then 0.5 mL of 1 M HCl was added and the mixture partitioned, when paper showed pH 4. The layers were separated and the aqueous layer extracted with 5:1 DCM/MeOH (2 mL). The residue was dissolved in 1:1 MeOH/ACN at 40 mg/mL and subjected to preparative SFC eluting a gradient of 5 mM NH₃ in methanol to CO₂ (15-50% over 10 min) though a 21.2×250 mm 2-PIC column, 5 μm particle, giving as a white solid (after evaporation of the collected fractions) the target: (14S,17R)-8-[(1E)-3,3-dimethylbut-1-en-1-yl]-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (29 mg, 72%). ¹H NMR (400 MHz, Chloroform-d) δ 8.61-8.56 (m, 1H), 7.96 (d, J=7.9 Hz, 1H), 7.66 (td, J=7.7, 1.8 Hz, 1H), 7.61-7.53 (m, 2H), 7.29 (d, J=7.7 Hz, 1H), 7.21 (ddd, J=7.6, 4.9, 1.1 Hz, 1H), 6.85 (d, J=15.8 Hz, 1H), 6.78 (d, J=7.9 Hz, 1H), 6.65 (dd, J=6.8, 2.4 Hz, 1H), 6.27 (d, J=15.8 Hz, 1H), 5.98 (d, J=8.2 Hz, 1H), 5.35 (q, J=6.9 Hz, 1H), 3.47 (dd, J=9.7, 6.8 Hz, 1H), 3.13 (s, 1H), 2.98 (t, J=10.1 Hz, 1H), 2.26 (dd, J=12.4, 9.1 Hz, 1H), 1.93 (dt, J=14.5, 7.5 Hz, 1H), 1.72 (d, J=9.1 Hz, 1H), 1.67 (s, 3H), 1.65 (s, 3H), 1.62-1.40 (m, 3H), 1.13 (s, 9H). ESI-MS m/z calc. 574.2726, found 575.5 (M+1)⁺; Retention time: 1.48 minutes (LC method S).

### Step 2: (14S,17R)-8-(3,3-dimethylbutyl)-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 105

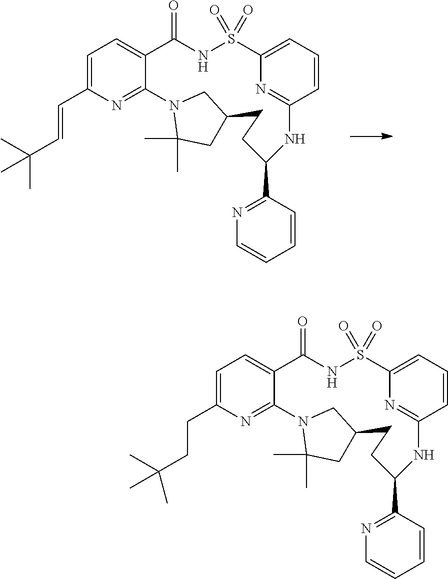 [SMILES: CC(C)(C)/C=C/c1ccc2c(n1)N1C[C@@H](CC[C@H](c3ccccn3)Nc3cccc(n3)S(=O)(=O)NC2=O)CC1(C)C ; CC(C)(C)CCc1ccc2c(n1)N1C[C@@H](CC[C@H](c3ccccn3)Nc3cccc(n3)S(=O)(=O)NC2=O)CC1(C)C]

A mixture of (14S,17R)-8-[(1E)-3,3-dimethylbut-1-en-1-yl]-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (24 mg, 0.04176 mmol), palladium on carbon (11 mg of 10% w/w, 0.01034 mmol), EtOAc (2 mL) and Methanol (2 mL) was stirred under balloon pressure of hydrogen for 16 h. Then the mixture was filtered, concentrated and the residue dissolved in EtOAc (2 mL) and Methanol (2 mL), Pd(OH)2 (7 mg of 20% w/w, 0.00997 mmol) was added and the mixture was subjected to Parr shaker hydrogenation at 55 psi of hydrogen for 2 h, filtered over Celite, solvent evaporated and the residue dissolved in MeOH and filtered through a 0.45 μm filter and the filtrate subjected to preparative SFC eluting a gradient of 5 mM NH₃ in methanol to CO₂ (15-50% over 10 min) though a 21.2×250 mm 2-PIC column, 5 μm particle, with a series of 400 μL injections giving as a white solid (after evaporation of the collected fractions) the target: (14S,17R)-8-(3,3-dimethyl butyl)-12,12-dimethyl-17-(pyridin-2-yl)-2λ⁶-thia-3,9,11,18,23-pentaazatetracyclo[17.3.1.111,14.05,10]tetracosa-1 (22),5,7,9,19(23),20-hexaene-2,2,4-trione (11.6 mg, 48%). ¹H NMR (400 MHz, Chloroform-d) δ 8.59-8.55 (m, 1H), 7.91 (d, J=7.8 Hz, 1H), 7.66 (td, J=7.7, 1.8 Hz, 1H), 7.59-7.51 (m, 2H), 7.29 (d, J=7.8 Hz, 1H), 7.21 (ddd, J=7.5, 4.8, 1.1 Hz, 1H), 6.70 (d, J=7.8 Hz, 1H), 6.65 (dd, J=7.4, 1.8 Hz, 1H), 5.96 (d, J=8.2 Hz, 1H), 5.35 (q, J=6.8 Hz, 1H), 3.45 (dd, J=9.6, 6.8 Hz, 1H), 3.08 (d, J=7.4 Hz, 1H), 2.97 (t, J=10.1 Hz, 1H), 2.68-2.60 (m, 2H), 2.23 (dd, J=12.4, 8.9 Hz, 1H), 1.92 (dt, J=14.5, 7.1 Hz, 1H), 1.70 (dt, J=14.3, 7.3 Hz, 1H), 1.63 (s, 6H), 1.61-1.41 (m, 5H), 0.95 (s, 9H). ESI-MS m/z calc. 576.28827, found 577.5 (M+1)⁺; Retention time: 1.53 minutes (LC method S).

### Example 53: Preparation of (14S,17R)-12,12-dimethyl-8-(4-methylpent-1-yn-1-yl)-1′7-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 157

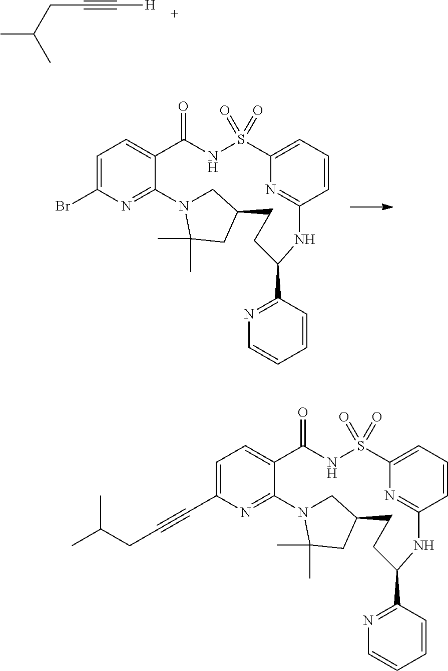

A mixture of (14S,17R)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (25 mg, 0.04375 mmol), Pd(PPh₃)₄ (10 mg, 0.0086 mmol), CuI (1.7 mg, 0.00893 mmol), triethylamine (0.43 mL) was degassed by bubbling nitrogen for 1 min then 4-methylpent-1-yne (103 μL, 0.8752 mmol) was added and the mixture heated to 100° C. for 16 h. Then the mixture was evaporated and dissolved with 2 mL water, 2 mL of 5:1 DCM/MeOH, then 0.6 mL of 1 M HCl added and the mixture partitioned, when paper showed pH 4. The layers were separated and the aqueous layer extracted with 5:1 DCM/MeOH (2 mL). The combined organic extracts were dried (MgSO₄) and evaporated. The residue was subjected to flash chromatography (12 g SiO2, 25-70% of a solution (5% MeOH in EtOAc) to hexanes) to provide target: (14S,17R)-12,12-dimethyl-8-(4-methylpent-1-yn-1-yl)-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (8.5 mg, 34%). ¹H NMR (400 MHz, Chloroform-d) δ 8.58-8.53 (m, 1H), 7.89 (d, J=7.9 Hz, 1H), 7.65 (td, J=7.7, 1.8 Hz, 1H), 7.56 (dd, J=8.2, 7.2 Hz, 1H), 7.52-7.48 (m, 1H), 7.29 (dd, J=7.8, 1.1 Hz, 1H), 7.20 (ddd, J=7.6, 4.9, 1.1 Hz, 1H), 6.87 (d, J=7.8 Hz, 1H), 6.66 (dd, J=8.2, 1.0 Hz, 1H), 5.93 (d, J=8.4 Hz, 1H), 5.35 (q, J=7.0 Hz, 1H), 3.47-3.39 (m, 1H), 3.02-2.91 (m, 2H), 2.32 (d, J=6.5 Hz, 2H), 2.17 (dd, J=11.7, 7.6 Hz, 1H), 2.01-1.87 (m, 2H), 1.74 (dt, J=14.5, 7.5 Hz, 1H), 1.63 (s, 3H), 1.62 (s, 3H), 1.60-1.37 (m, 3H), 1.05 (d, J=6.7 Hz, 6H). ESI-MS m/z calc. 572.25696, found 573.0 (M+1)⁺; Retention time: 1.4 minutes (LC method A).

### Example 54: Preparation of (14S,17R)-12,12-dimethyl-17-(pyridin-2-yl)-8-[(1s,3S)-3-(propan-2-yloxy)cyclobutoxy]-2λ⁶-thia-3,9,11,18,23-pentaazatetracyclo [17.3.1.111,14.05,10]tetracosa-1(22),5,7,9,19(23),20-hexaene-2,2,4-trione, Compound 161

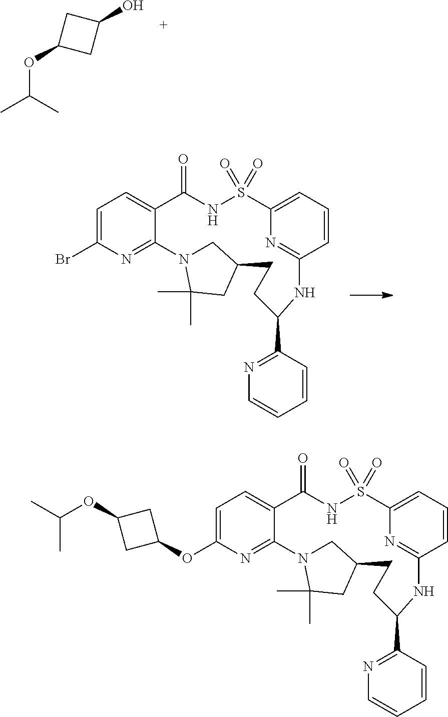

To a mixture of (14S,17R)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (25 mg, 0.04375 mmol), 3-isopropoxycyclobutanol (17 mg, 0.1306 mmol), sodium tert-butoxide (13 mg, 0.1353 mmol), Pd₂(dba)₃ (4 mg, 0.004368 mmol), and [1-(2-diphenylphosphanyl-1-naphthyl)-2-naphthyl]-diphenyl-phosphane (3 mg, 0.004818 mmol) was added toluene (0.43 mL), the mixture degassed by bubbling in nitrogen for 30 s, then heated at 100° C. for 16 h and additional Pd₂(dba)₃ (4 mg, 0.004368 mmol) and [1-(2-diphenylphosphanyl-1-naphthyl)-2-naphthyl]-diphenyl-phosphane (3 mg, 0.0048 mmol) added. Heating at 100° C. was continued 24 h, then the mixture was diluted with EtOAc, pH lowered to 4 with 1 M HCl, extracted with 4-1 DCM/MeOH, dried and evaporated. The residue was dissolved into 1:1 ACN/MeOH and purified by preparative SFC eluting a gradient of 5 mM NH₃ in methanol to CO₂ (25-50% over 10 min) though a 21.2×250 mm 2-PIC column (column 6), 5 μm particle, to provide (14S,17R)-12,12-dimethyl-17-(pyridin-2-yl)-8-[(1s,3S)-3-(propan-2-yloxy)cyclobutoxy]-2λ⁶-thia-3,9,11,18,23-pentaazatetracyclo[17.3.1.111,14.05,10]tetracosa-1(22),5,7,9,19(23),20-hexaene-2,2,4-trione (2.0 mg, 7%). ¹H NMR (400 MHz, Chloroform-d) δ 10.17 (s, 1H), 8.63-8.55 (m, 1H), 7.91 (d, J=8.5 Hz, 1H), 7.74-7.64 (m, 1H), 7.57 (dd, J=8.0, 7.3 Hz, 1H), 7.52 (dd, J=7.3, 1.1 Hz, 1H), 7.31 (d, J=7.8 Hz, 1H), 7.26-7.15 (m, 1H), 6.66 (dd, J=8.0, 1.2 Hz, 1H), 6.24 (d, J=8.5 Hz, 1H), 5.94 (d, J=8.4 Hz, 1H), 5.34 (q, J 7.0 Hz, 1H), 4.83 (p, J=7.2 Hz, 1H), 3.79 (p, J=7.1 Hz, 1H), 3.61 (p, J=6.1 Hz, 1H), 3.01 (d, J=4.4 Hz, 2H), 2.85 (ddt, J=14.3, 11.9, 5.8 Hz, 2H), 2.17 (ddt, J=18.0, 12.6, 7.4 Hz, 3H), 1.92 (dt, J=14.7, 7.5 Hz, 1H), 1.75 (d, J=7.5 Hz, 1H), 1.60 (t, J=6.7 Hz, 10H), 1.15 (dd, J=6.1, 0.9 Hz, 6H). ESI-MS m/z calc. 620.2781, found 621.2 (M+1)⁺; Retention time: 1.25 minutes (LC method A).

### Example 55: Preparation of (14S,17R)-12,12-dimethyl-2,2,4-trioxo-17-(pyridin-2-yl)-2λ⁶-thia-3,9,11,18,23-pentaazatetracyclo[17.3.1.111,14.05,10]tetracosa-1(22),5,7,9,19(23),20-hexaene-8-carbonitrile, Compound 193

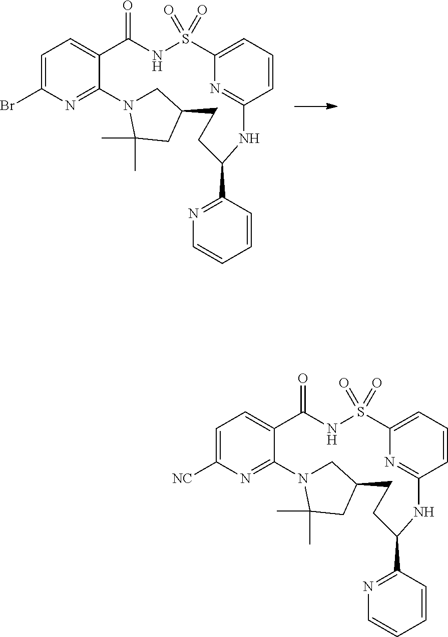 [SMILES: CC1(C)C[C@@H]2CC[C@H](c3ccccn3)Nc3cccc(n3)S(=O)(=O)NC(=O)c3ccc(Br)nc3N1C2 ; CC1(C)C[C@@H]2CC[C@H](c3ccccn3)Nc3cccc(n3)S(=O)(=O)NC(=O)c3ccc(C#N)nc3N1C2]

A mixture of (14S)-8-bromo-12,12-dimethyl-17-(pyridin-2-yl)-2λ⁶-thia-3,9,11,18,23-pentaazatetracyclo[17.3.1.111,14.05,10]tetracosa-1(22),5(10),6,8,19(23),20-hexaene-2,2,4-trione (46 mg, 0.08049 mmol), dicyanozinc (19 mg, 0.1618 mmol), and Pd(PPh₃)₄ (9 mg, 0.00779 mmol) in DMF (0.41 mL) was bubbled with nitrogen for 1 min and then heated at 100° C. for 1 h. The mixture was diluted with EtOAc, washed with water, dried (MgSO₄) and evaporated to provide a residue which was purified by SFC eluting a gradient of 5 mM NH₃ in methanol to CO₂ (50-80% over 10 min) though a 21.2×250 mm OD-3 column, 5 μm particle, to provide (14S,17R)-12,12-dimethyl-2,2,4-trioxo-17-(pyridin-2-yl)-2λ⁶-thia-3,9,11,18,23-pentaazatetracyclo[17.3.1.111,14.05,10]tetracosa-1(22),5,7,9,19(23),20-hexaene-8-carbonitrile (7.9 mg, 19%). ¹H NMR (500 MHz, DMSO-d₆) δ 8.68-8.41 (m, 1H), 7.81 (d, J=7.6 Hz, 1H), 7.73 (td, J=7.7, 1.8 Hz, 1H), 7.60 (dd, J=16.9, 9.5 Hz, 2H), 7.43 (d, J=7.9 Hz, 1H), 7.23 (dd, J=7.5, 4.8 Hz, 1H), 7.12 (d, J 7.6 Hz, 1H), 7.07 (d, J=7.2 Hz, 1H), 6.83 (d, J=8.6 Hz, 1H), 5.28 (t, J=10.4 Hz, 1H), 3.35 (s, 1H), 2.83 (s, 1H), 2.23 (s, 1H), 1.98 (t, J=12.8 Hz, 1H), 1.88 (td, J=14.9, 9.6 Hz, 2H), 1.76-1.67 (m, 1H), 1.59 (s, 4H), 1.47 (s, 4H). ESI-MS m/z calc. 517.18964, found 518.0 (M+1)⁺; Retention time: 1.02 minutes (LC method A).

### Example 56: Preparation of various analogs of (14S,17R)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 246

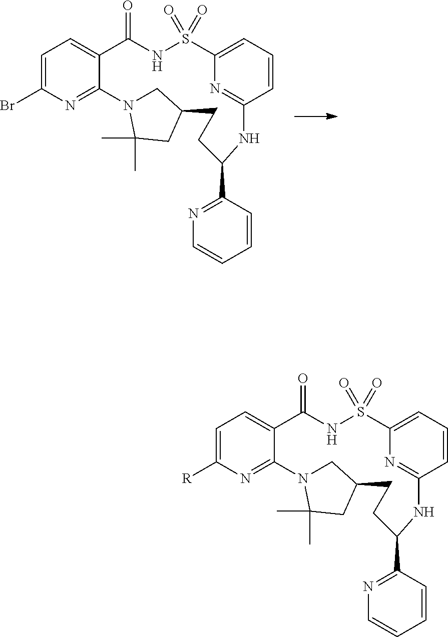

Benzyl(trifluoro)boranuide (potassium salt) is a boron reagent that is commercially available.

3,3-Dimethylbut-1-yne is an alkyne reagent that is commercially available:

The compounds in the following tables were prepared in a manner analogous to that described above using boron and alkyne reagents given in the table above, and by using (14S,17R)-8-bromo-12,12-dimethyl-17-(pyridin-2-yl)-2λ⁶-thia-3,9,11,18,23-pentaazatetracyclo[17.3.1.111,14.05,10]tetracosa-1 (22),5,7,9,19(23),20-hexaene-2,2,4-trione, Compound 246, as a starting material.

| | | LCMS | | | |
|---|---|---|---|---|---|
| | | Retention | | | |
| Compound | | Time | Exact | | LCMS |
| Number | Structure | (min) | Mass | M + 1 | Method |
| Compound 159 | 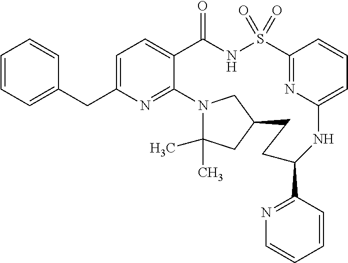 [SMILES: CC1(C)C[C@@H]2CC[C@H](c3ccccn3)Nc3cccc(n3)S(=O)(=O)NC(=O)c3ccc(Cc4ccccc4)nc3N1C2] | 1.32 | 582.241 | 583.4 | LC method A |
| Compound 177 | 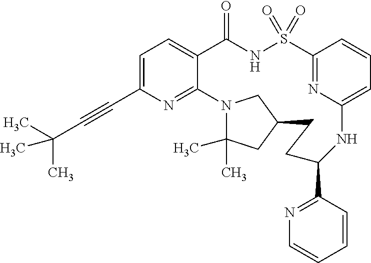 [SMILES: CC(C)(C)C#Cc1ccc2c(n1)N1C[C@@H](CC[C@H](c3ccccn3)Nc3cccc(n3)S(=O)(=O)NC2=O)CC1(C)C] | 1.4 | 572.257 | 573 | LC method A |

| Compound | | |
|---|---|---|
| Number | Structure | NMR |
| Compound 159 | 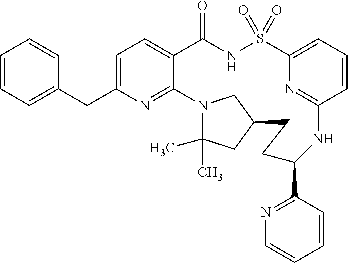 [SMILES: CC1(C)C[C@@H]2CC[C@H](c3ccccn3)Nc3cccc(n3)S(=O)(=O)NC(=O)c3ccc(Cc4ccccc4)nc3N1C2] | ¹H NMR (400 MHz, Chloroform-d) δ 8.56 (d, J = 4.8 Hz, 1H), 7.88 (d, J = 7.8 Hz, 1H), 7.65 (td, J = 7.6, 1.8 Hz, 1H), 7.56 (d, J = 7.4 Hz, 1H), 7.52 (t, J = 5.6 Hz, 1H), 7.29 (td, J = 7.6, 1.9 Hz, 3H), 7.23 (dd, J = 6.8, 1.6 Hz, 2H), 7.21-7.17 (m, 2H), 6.66 (t, J = 7.6 Hz, 2H), 5.92 (d, J = 8.3 Hz, 1H), 5.33 (q, J = 7.1 Hz, 1H), 4.01 (s, 2H), 3.43 (s, 1H), 2.96 (d, J = 9.2 Hz, 2H), 2.17 (dd, J = 12.3, 7.9 Hz, 1H), 1.91 (dt, J= 14.5, 7.4 Hz, 1H), 1.72 (dt, J = 15.4, 8.2 Hz, 1H), 1.61 (d, J = 5.7 Hz, 1H), 1.57 (d, J = 2.4 Hz, 1H), 1.52 (s, 6H), 1.48 (s, 1H). |
| Compound 177 | 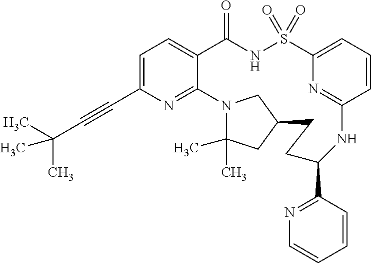 [SMILES: CC(C)(C)C#Cc1ccc2c(n1)N1C[C@@H](CC[C@H](c3ccccn3)Nc3cccc(n3)S(=O)(=O)NC2=O)CC1(C)C] | ¹H NMR (400 MHz, Chloroform-d) δ 8.56 (d, J = 4.9 Hz, 1H), 7.85 (d, J = 7.9 Hz, 1H), 7.73 (t, J = 7.6 Hz, 1H), 7.67 (dd, J = 11.7, 7.6 Hz, 1H), 7.56 (t, J = 7.7 Hz, 1H), 7.49 (d, J = 7.2 Hz, 1H), 7.45 (t, J = 8.6 Hz, 1H), 7.28 (d, J = 6.7 Hz, 1H), 6.83 (d, J = 7.8 Hz, 1H), 6.70 (d, J = 8.3 Hz, 1H), 6.10 (s, 1H), 5.45 (d, J = 7.6 Hz, 1H), 3.50-3.42 (m, 1H), 2.94 (t, J = 9.5 Hz, 2H), 2.13 (dd, J = 12.4, 7.2 Hz, 1H), 1.97-1.76 (m, 1H), 1.63 (s, 3H), 1.61 (s, 3H), 1.59-1.40 (m, 2H), 1.33 (s, 9H). |

### Example 57: Preparation of various analogs of (14S,17S)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 247

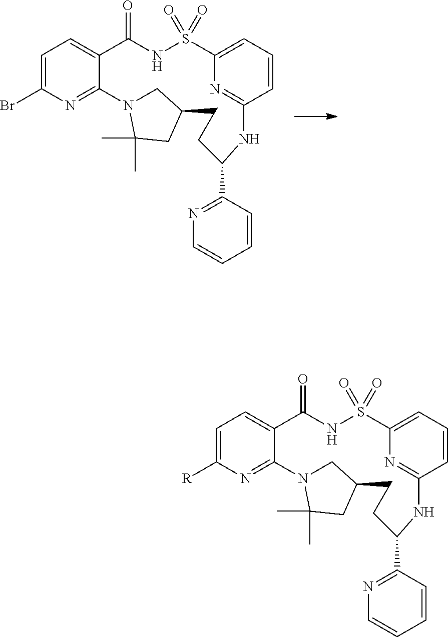

The compounds in the following tables were prepared in a manner analogous to that described above, using (14S,17S)-8-bromo-12,12-dimethyl-17-(pyridin-2-yl)-2λ⁶-thia-3,9,11,18,23-pentaazatetracyclo[17.3.1.111,14.05,10]tetracosa-1 (22),5,7,9,19(23),20-hexaene-2,2,4-trione, Compound 247, as a starting material:

| Compound | | | | | |
|---|---|---|---|---|---|
| Number | Structure | NMR | | | |
| Compound 158 | 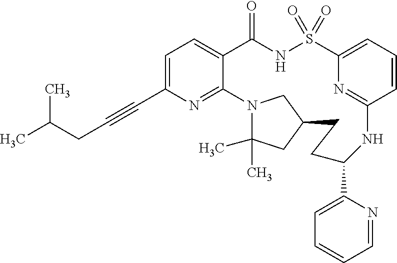 [SMILES: CC(C)CC#Cc1ccc2c(n1)N1C[C@@H](CC[C@@H](c3ccccn3)Nc3cccc(n3)S(=O)(=O)NC2=O)CC1(C)C] | ¹H NMR (400 MHz, Chloroform-d) δ 11.82 (s, 1H), 8.57 (s, 1H), 8.11 (d, J = 8.0 Hz, 1H), 7.74-7.66 (m, 1H), 7.65-7.56 (m, 2H), 7.28 (s, 1H), 7.24 (s, 1H), 7.05 (d, J = 7.9 Hz, 1H), 6.77 (s, 1H), 6.75 (s, 1H), 4.76 (s, 1H), 3.42 (dd, J = 10.0, 5.0 Hz, 1H), 3.28 (s, 1H), 2.46 (s, 1H), 2.33 (d, J = 6.5 Hz, 2H), 2.05 (dd, J = 12.3, 7.8 Hz, 1H), 1.94 (dt, J = 13.2, 6.6 Hz, 1H), 1.83 (s, 2H), 1.76 (t, J = 10.9 Hz, 1H), 1.58 (s, 3H), 1.56 (s, 3H), 1.49-1.39 (m, 2H), 1.05 (d, J = 6.7 Hz, 6H). | | | |
| Compound 160 | 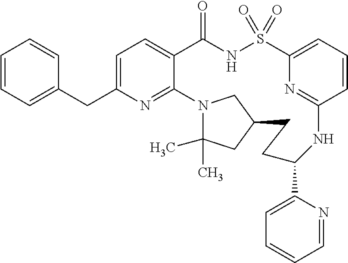 [SMILES: CC1(C)C[C@@H]2CC[C@@H](c3ccccn3)Nc3cccc(n3)S(=O)(=O)NC(=O)c3ccc(Cc4ccccc4)nc3N1C2] | ¹H NMR (400 MHz, Chloroform-d) δ 11.55 (s, 1H), 8.61-8.52 (m, 1H), 8.08 (d, J = 7.9 Hz, 1H), 7.70 (td, J = 7.7, 1.8 Hz, 1H), 7.63 (d, J = 7.2 Hz, 1H), 7.61-7.56 (m, 1H), 7.32- 7.27 (m, 2H), 7.22 (d, J = 6.5 Hz, 3H), 6.83 (d, J = 7.9 Hz, 1H), 6.75 (d, J = 7.7 Hz, 2H), 4.75 (s, 1H), 4.04 (s, 2H), 3.44 (dd, J = 9.9, 5.0 Hz, 1H), 3.26 (s, 1H), 2.43 (s, 1H), 2.31 (s, 1H), 2.02 (dd, J = 12.1, 7.8 Hz, 1H), 1.94 (t, J = 8.9 Hz, 1H), 1.83 (d, J = 8.4 Hz, 1H), 1.73 (t, J = 10.9 Hz, 1H), 1.60 (s, 3H), 1.47 (d, J = 2.9 Hz, 6H). | | | |
| Compound 162 | 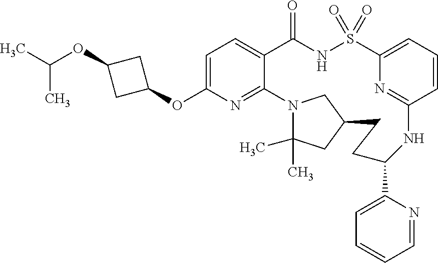 [SMILES: CC(C)O[C@H]1C[C@@H](Oc2ccc3c(n2)N2C[C@@H](CC[C@@H](c4ccccn4)Nc4cccc(n4)S(=O)(=O)NC3=O)CC2(C)C)C1] | ¹H NMR (400 MHz, Chloroform-d) δ 11.65 (s, 1H), 8.58 (ddd, J = 4.9, 1.9, 0.9 Hz, 1H), 8.10 (d, J = 8.6 Hz, 1H), 7.71 (t, J = 7.5 Hz, 1H), 7.65-7.62 (m, 1H), 7.62-7.57 (m, 1H), 7.29 (s, 1H), 7.24 (d, J = 6.5 Hz, 1H), 6.75 (dd, J = 8.0, 1.2 Hz, 1H), 6.40 (d, J = 8.6 Hz, 1H), 4.83 (p, J = 7.2 Hz, 1H), 4.75 (s, 1H), 3.79 (p, J = 7.1 Hz, 1H), 3.61 (q, J = 6.1 Hz, 1H), 3.42 (dd, J = 10.0, 5.2 Hz, 1H), 3.27 (s, 1H), 2.85 (ddt, J = 13.9, 12.1, 6.0 Hz, 2H), 2.51 (s, 1H), 2.33 (s, 1H), 2.21-2.03 (m, 2H), 1.96 (t, J = 9.4 Hz, 1H), 1.78 (dd, J = 23.2, 12.3 Hz, 2H), 1.56-1.53 (m, 9H), 1.15 (dd, J = 6.1, 1.1Hz, 6H). | | | |
| Compound 176 | 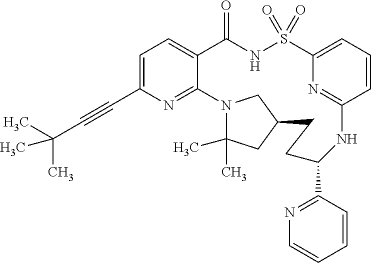 [SMILES: CC(C)(C)C#Cc1ccc2c(n1)N1C[C@@H](CC[C@@H](c3ccccn3)Nc3cccc(n3)S(=O)(=O)NC2=O)CC1(C)C] | ¹H NMR (400 MHz, Chloroform-d) δ 11.94 (s, 1H), 8.57 (d, J = 4.6 Hz, 1H), 8.11 (d, J = 7.9 Hz, 1H), 7.73-7.67 (m, 1H), 7.64 (d, J = 7.1 Hz, 1H), 7.60 (t, J = 7.6 Hz, 1H), 7.28 (s, 1H), 7.24 (dd, J = 7.7, 5.1 Hz, 1H), 7.05 (d, J = 8.0 Hz, 1H), 6.78-6.72 (m, 2H), 4.75 (s, 1H), 3.40 (dd, J = 9.9, 5.1 Hz, 1H), 3.29 (d, J = 9.4 Hz, 1H), 2.48 (s, 1H), 2.34 (s, 1H), 2.11-2.00 (m, 1H), 1.99-1.90 (m, 1H), 1.88- 1.60 (m, 2H), 1.58 (s, 3H), 1.55 (s, 3H), 1.51-1.34 (m, 1H), 1.33 (s, 9H). | | | |
| Compound 192 | 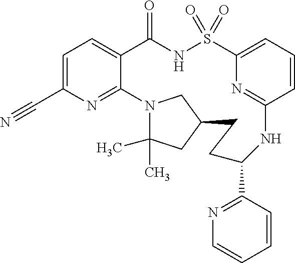 [SMILES: CC1(C)C[C@@H]2CC[C@@H](c3ccccn3)Nc3cccc(n3)S(=O)(=O)NC(=O)c3ccc(C#N)nc3N1C2] | ¹H NMR (500 MHz, DMSO-d₆) δ 8.57 (d, J = 4.9 Hz, 1H), 7.76 (t, J = 7.7 Hz, 1H), 7.56 (s, 3H), 7.33-7.25 (m, 1H), 7.17 (d, J = 7.3 Hz, 1H), 7.08 (d, J = 7.6 Hz, 1H), 6.79 (d, J = 8.2 Hz, 1H), 4.89 (s, 1H), 2.28 (s, 2H), 2.03 (s, 2H), 1.85 (dd, J = 12.0, 5.9 Hz, 1H), 1.66 (s, 2H), 1.57 (s, 4H), 1.46 (s, 3H), 1.17 (s, 1H) | | | |
| | | LCMS | | | |
| Compound | | Retention | Exact | | LCMS |
| Number | Structure | Time (min) | Mass | M + 1 | Method |
| Compound 158 | 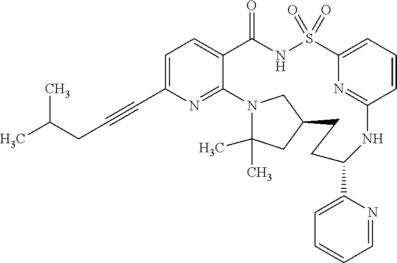 [SMILES: CC(C)CC#Cc1ccc2c(n1)N1C[C@@H](CC[C@@H](c3ccccn3)Nc3cccc(n3)S(=O)(=O)NC2=O)CC1(C)C] | 1.35 | 572.257 | 573 | LC method A |
| Compound 160 | 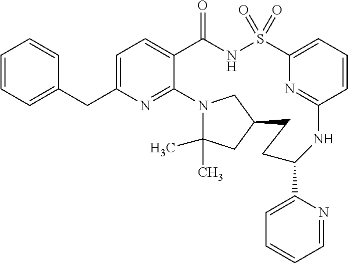 [SMILES: CC1(C)C[C@@H]2CC[C@@H](c3ccccn3)Nc3cccc(n3)S(=O)(=O)NC(=O)c3ccc(Cc4ccccc4)nc3N1C2] | 1.22 | 582.241 | 583.5 | LC method A |
| Compound 162 | 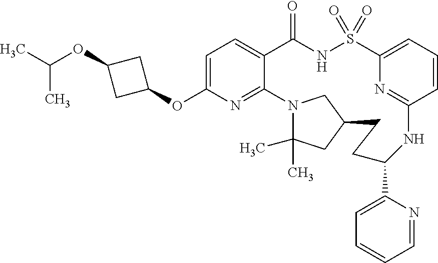 [SMILES: CC(C)O[C@H]1C[C@@H](Oc2ccc3c(n2)N2C[C@@H](CC[C@@H](c4ccccn4)Nc4cccc(n4)S(=O)(=O)NC3=O)CC2(C)C)C1] | 1.21 | 620.278 | 621.2 | LC method A |
| Compound 176 | 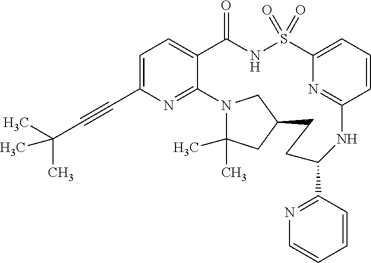 [SMILES: CC(C)(C)C#Cc1ccc2c(n1)N1C[C@@H](CC[C@@H](c3ccccn3)Nc3cccc(n3)S(=O)(=O)NC2=O)CC1(C)C] | 1.34 | 572.257 | 573.2 | LC method A |
| Compound 192 | 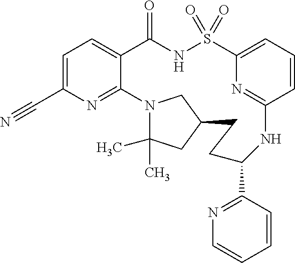 [SMILES: CC1(C)C[C@@H]2CC[C@@H](c3ccccn3)Nc3cccc(n3)S(=O)(=O)NC(=O)c3ccc(C#N)nc3N1C2] | 0.96 | 517.19 | 518 | LC method A |

### Example 58: Preparation of (14S,17R)-8-bromo-12,12-dimethyl-17-[6-(trifluoromethyl)pyridin-2-yl]-2λ⁶-thia-3,9,11,18,23-pentaazatetracyclo [17.3.1.111,14.05,10]tetracosa-1(22),5,7,9,19(23),20-hexaene-2,2,4-trione, Compound 56, and (14S,17S)-8-bromo-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione

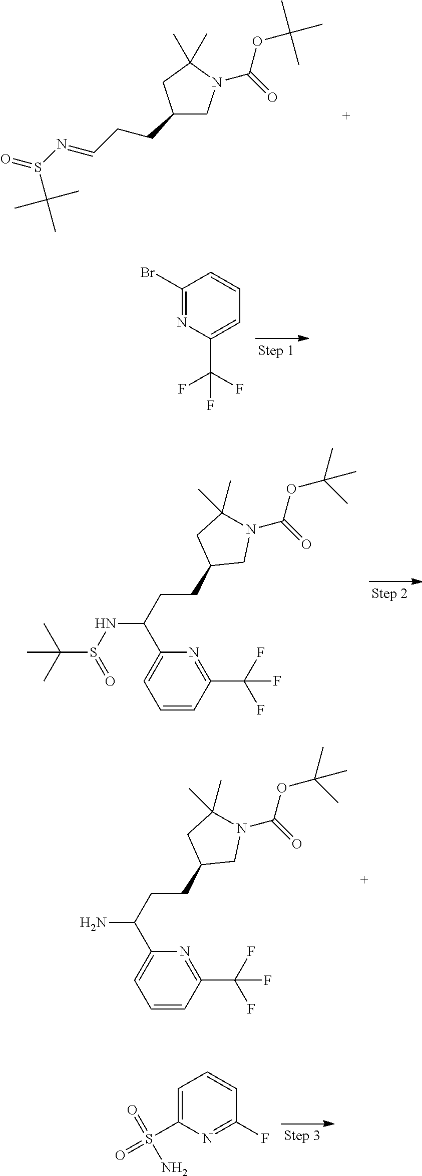

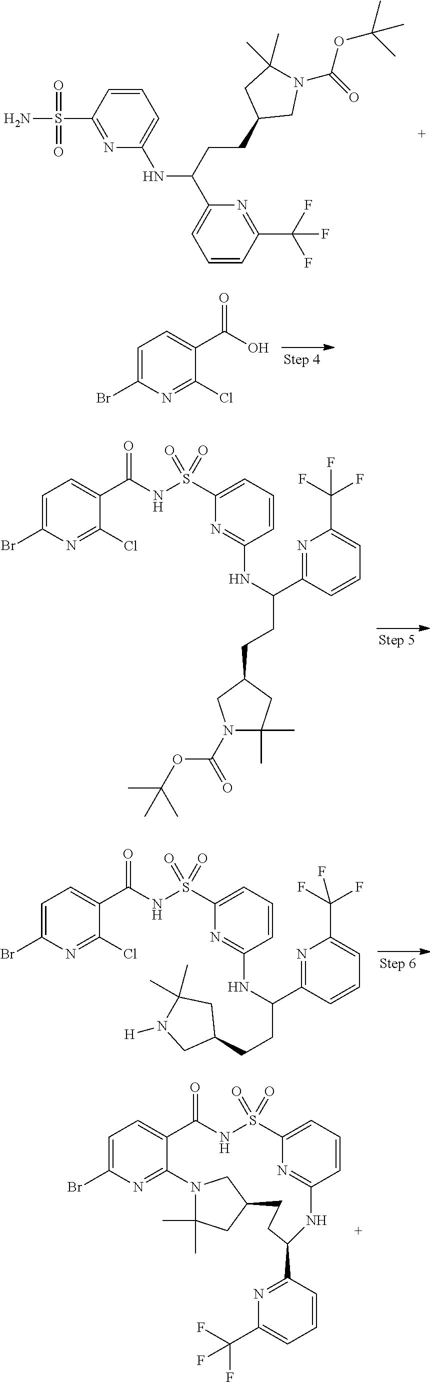

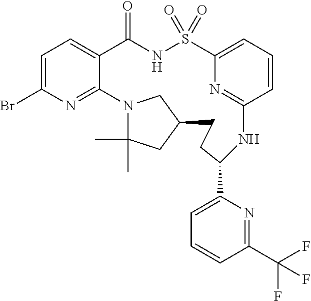 [SMILES: CC1(C)C[C@@H]2CC[C@@H](c3cccc(C(F)(F)F)n3)Nc3cccc(n3)S(=O)(=O)NC(=O)c3ccc(Br)nc3N1C2]

### Step 1: tert-Butyl (4S)-4-[3-(tert-butylsulfinylamino)-3-[6-(trifluoromethyl)-2-pyridyl]propyl]-2,2-dimethyl-pyrrolidine-1-carboxylate

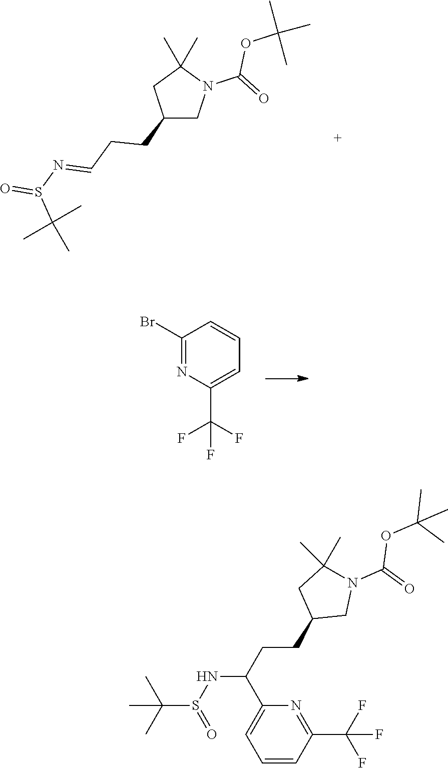

2-Bromo-6-(trifluoromethyl)pyridine (6.6 g, 28.62 mmol) was dissolved in diethyl ether (100 mL). The solution was cooled in a dry ice acetone bath and stirred under nitrogen balloon for 15 min. n-BuLi (11.5 mL of 2.5 M in hexanes, 28.75 mmol) was added within 1 min. The light yellowish solution was stirred below −70° C. for 45 min. tert-Butyl (4S)-4-[(3E)-3-tert-butylsulfinyliminopropyl]-2,2-dimethyl-pyrrolidine-1-carboxylate (5.4 g, 14.31 mmol) was then added as a solution of THF (8 mL plus 2 mL rinse). The mixture was further stirred at the same temperature for 45 min. NH₄Cl (40 mL, saturated aqueous) was added, followed by water (50 mL) and EtOAc (100 mL). The mixture was allowed to warm up to rt and combined with another batch of crude material. The layers were separated and the organic layer was washed with brine, dried over anhydrous MgSO₄, filtered and concentrated. The residue was purified by silica gel chromatography (80 g column), using 10-80% EtOAc in hexanes to afford tert-butyl (4S)-4-[3-(tert-butylsulfinylamino)-3-[6-(trifluoromethyl)-2-pyridyl]propyl]-2,2-dimethyl-pyrrolidine-1-carboxylate (6.96 g, 91%) as a glassy solid. ESI-MS m/z calc. 505.2586, found 506.6 (M+1)⁺; Retention time: 3.79 minutes (LC method B).

### Step 2: tert-Butyl (4S)-4-[3-amino-3-[6-(trifluoromethyl)-2-pyridyl]propyl]-2,2-dimethyl-pyrrolidine-1-carboxylate

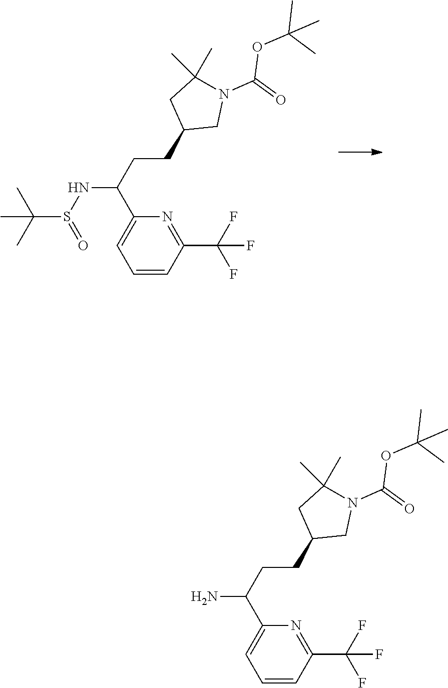 [SMILES: CC(C)(C)OC(=O)N1C[C@@H](CCC(NS(=O)C(C)(C)C)c2cccc(C(F)(F)F)n2)CC1(C)C ; CC(C)(C)OC(=O)N1C[C@@H](CCC(N)c2cccc(C(F)(F)F)n2)CC1(C)C]

tert-Butyl (4S)-4-[3-(tert-butylsulfinylamino)-3-[6-(trifluoromethyl)-2-pyridyl]propyl]-2,2-dimethyl-pyrrolidine-1-carboxylate (7.45 g, 13.997 mmol) was dissolved in a mixture of THF (100 mL) and Water (20 mL). Molecular iodine (1.05 g, 4.14 mmol) was added. The mixture was then heated in a 50° C. oil bath and stirred 3 h. It was cooled to rt, treated with Na₂S₂O₃ (5 g in 50 mL of saturated aqueous sodium bicarbonate) and diluted with EtOAc (100 mL). The layers were separated and the organic layer was washed with brine, dried over anhydrous MgSO₄, filtered and concentrated. The residue was purified by silica gel chromatography (80 g column), using 0-10% MeOH in DCM to afford tert-butyl (4S)-4-[3-amino-3-[6-(trifluoromethyl)-2-pyridyl]propyl]-2,2-dimethyl-pyrrolidine-1-carboxylate as a light brown foam. (4.6 g, 78%). ESI-MS m/z calc. 401.229, found 402.6 (M+1)⁺; Retention time: 2.86 minutes. (LC method B).

### Step 3: tert-Butyl (4S)-2,2-dimethyl-4-[3-[(6-sulfamoyl-2-pyridyl)amino]-3-[6-(trifluoromethyl)-2-pyridyl]propyl]pyrrolidine-1-carboxylate

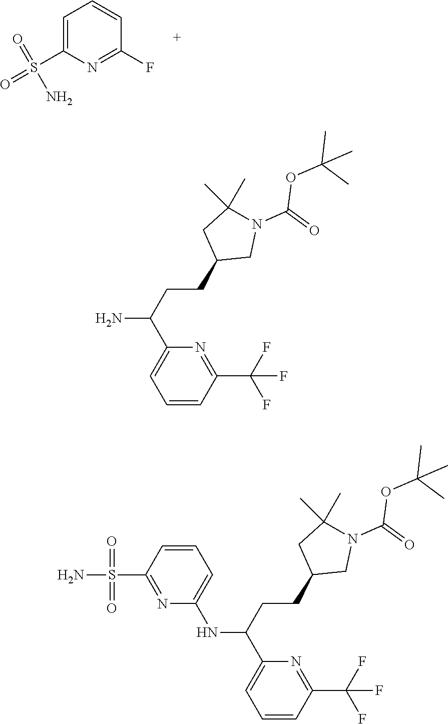 [SMILES: NS(=O)(=O)c1cccc(F)n1 ; CC(C)(C)OC(=O)N1C[C@@H](CCC(N)c2cccc(C(F)(F)F)n2)CC1(C)C ; CC(C)(C)OC(=O)N1C[C@@H](CCC(Nc2cccc(S(N)(=O)=O)n2)c2cccc(C(F)(F)F)n2)CC1(C)C]

tert-Butyl (4S)-4-[3-amino-3-[6-(trifluoromethyl)-2-pyridyl]propyl]-2,2-dimethyl-pyrrolidine-1-carboxylate (4.6 g, 10.88 mmol) was dissolved in DMSO (6 mL). 6-Fluoropyridine-2-sulfonamide (1.92 g, 10.90 mmol) was added in one portion, followed by Na₂CO₃ (3.5 g, 33.02 mmol). The mixture was placed in a pre-heated 110° C. oil bath and stirred under a nitrogen balloon for 20 h. It was then cooled to rt, diluted with water (30 mL) and EtOAc (50 mL). The layers were separated and the organic layer was washed with more water (30 mL), brine (30 mL), dried over anhydrous MgSO₄, filtered and concentrated. The residue was purified by silica gel chromatography (120 g column), using 5-50% EtOAc in hexanes to afford tert-butyl (4S)-2,2-dimethyl-4-[3-[(6-sulfamoyl-2-pyridyl)amino]-3-[6-(trifluoromethyl)-2-pyridyl]propyl]pyrrolidine-1-carboxylate as a light brownish foam (3.7738 g, 60%). ¹H NMR (500 MHz, DMSO-d₆) δ 8.01 (t, J=7.8 Hz, 1H), 7.87 (td, J=9.3, 8.4, 2.0 Hz, 1H), 7.74 (d, J=7.6 Hz, 1H), 7.64 (d, J=8.4 Hz, 1H), 7.57-7.50 (m, 1H), 7.07 (s, 2H), 6.98 (d, J=7.2 Hz, 1H), 6.73 (d, J=8.2 Hz, 1H), 5.26 (s, 1H), 3.52 (dt, J=12.0, 6.7 Hz, 1H), 2.76 (dtd, J=25.7, 10.6, 5.3 Hz, 1H), 2.16-2.02 (m, 1H), 1.98-1.78 (m, 3H), 1.49-1.28 (m, 15H), 1.22 (s, 3H). ESI-MS m/z calc. 557.2284, found 558.4 (M+1)⁺; Retention time: 2.84 minutes (LC method H).

### Step 4: tert-Butyl (4S)-4-[3-[[6-[(6-bromo-2-chloro-pyridine-3-carbonyl)sulfamoyl]-2-pyridyl]amino]-3-[6-(trifluoromethyl)-2-pyridyl]propyl]-2,2-dimethyl-pyrrolidine-1-carboxylate

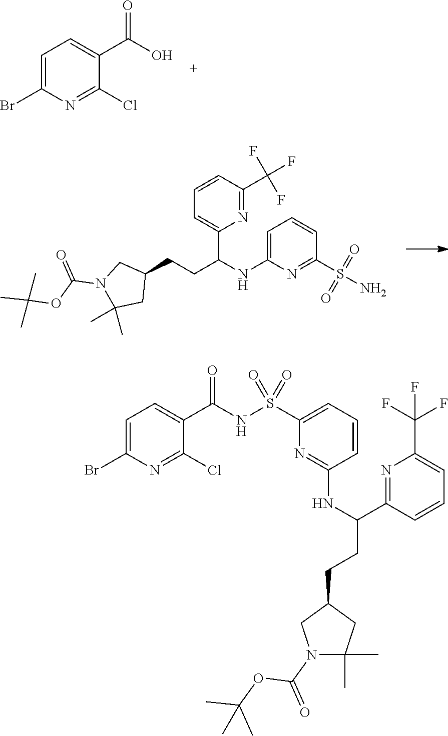 [SMILES: O=C(O)c1ccc(Br)nc1Cl ; CC(C)(C)OC(=O)N1C[C@@H](CCC(Nc2cccc(S(N)(=O)=O)n2)c2cccc(C(F)(F)F)n2)CC1(C)C ; CC(C)(C)OC(=O)N1C[C@@H](CCC(Nc2cccc(S(=O)(=O)NC(=O)c3ccc(Br)nc3Cl)n2)c2cccc(C(F)(F)F)n2)CC1(C)C]

A mixture of 6-bromo-2-chloro-pyridine-3-carboxylic acid (510 mg, 2.16 mmol) and CDI (350 mg, 2.16 mmol) were dissolved in THF (3.6 mL) and the mixture stirred at 60° C. for 45 min then tert-butyl (4S)-2,2-dimethyl-4-[3-[(6-sulfamoyl-2-pyridyl)amino]-3-[6-(trifluoromethyl)-2-pyridyl]propyl]pyrrolidine-1-carboxylate (600 mg, 1.076 mmol) was added followed by DBU (726 μL, 4.85 mmol) and the resulting mixture was stirred at rt for 4 h. The mixture was diluted with EtOAc and washed with 1 N HCl in water, water, brine, dried (MgSO₄), and concentrated. The residue was purified (flash chromatography: 40 g SiO2, 10-35% EtOAc in hexanes) to provide the desired product: tert-butyl (4S)-4-[3-[[6-[(6-bromo-2-chloro-pyridine-3-carbonyl)sulfamoyl]-2-pyridyl]amino]-3-[6-(trifluoromethyl)-2-pyridyl]propyl]-2,2-dimethyl-pyrrolidine-1-carboxylate (590 mg, 71%). ¹H NMR (400 MHz, Chloroform-d) δ 7.89 (dq, J=13.7, 7.5, 6.8 Hz, 1H), 7.72 (dd, J=13.6, 8.0 Hz, 1H), 7.63 (d, J=7.8 Hz, 1H), 7.60-7.51 (m, 2H), 7.45 (ddd, J 17.1, 10.3, 6.5 Hz, 2H), 6.74-6.65 (m, 1H), 5.82 (d, J 9.4 Hz, 1H), 5.35 (d, J=7.3 Hz, 1H), 5.16 (s, 1H), 4.50 (s, 1H), 4.20-4.08 (m, 1H), 3.11 (q, J=10.2 Hz, 1H), 2.37 (s, OH), 2.19 (d, J 12.1 Hz, 1H), 2.05 (s, 1H), 1.81 (td, J 13.0, 5.8 Hz, 1H), 1.67-1.54 (m, 11H), 1.45 (dd, J=14.7, 10.4 Hz, 1H), 1.39 (s, 5H), 1.35 (s, 4H), 1.34-1.21 (m, 7H). ESI-MS m/z calc. 774.12134, found 776.9 (M+1)⁺; Retention time: 0.8 minutes (LC method D).

### Step 5: 6-Bromo-2-chloro-N-[[6-[[3-[(3R)-5,5-dimethylpyrrolidin-3-yl]-1-[6-(trifluoromethyl)-2-pyridyl]propyl]amino]-2-pyridyl]sulfonyl]pyridine-3-carboxamide

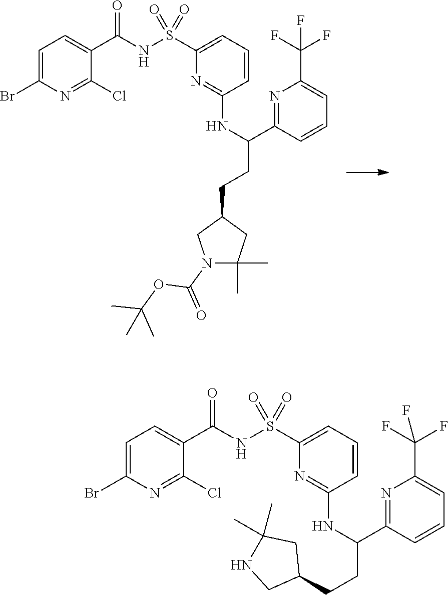 [SMILES: CC(C)(C)OC(=O)N1C[C@@H](CCC(Nc2cccc(S(=O)(=O)NC(=O)c3ccc(Br)nc3Cl)n2)c2cccc(C(F)(F)F)n2)CC1(C)C ; CC1(C)C[C@@H](CCC(Nc2cccc(S(=O)(=O)NC(=O)c3ccc(Br)nc3Cl)n2)c2cccc(C(F)(F)F)n2)CN1]

To a solution of tert-butyl (4S)-4-[3-[[6-[(6-bromo-2-chloro-pyridine-3-carbonyl)sulfamoyl]-2-pyridyl]amino]-3-[6-(trifluoromethyl)-2-pyridyl]propyl]-2,2-dimethyl-pyrrolidine-1-carboxylate (590 mg, 0.7602 mmol) in DCM (4 mL) was added TFA (1.8 mL, 23.36 mmol) and the mixture was stirred at rt for 1 h. The solution was concentrated to dryness under reduced pressure, then co-evaporated with THF (2×20 mL) and dried under high vacuum at rt for 16 h to provide: 6-bromo-2-chloro-N-[[6-[[3-[(3R)-5,5-dimethylpyrrolidin-3-yl]-1-[6-(trifluoromethyl)-2-pyridyl]propyl]amino]-2-pyridyl]sulfonyl]pyridine-3-carboxamide (trifluoroacetate salt) (772 mg, 100%). ESI-MS m/z calc. 674.0689, found 676.9 (M+1)⁺; Retention time: 0.5 minutes (LC method D).

### Step 6: (14S,17R)-8-Bromo-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione, Compound 56, and (14S,17S)-8-bromo-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione

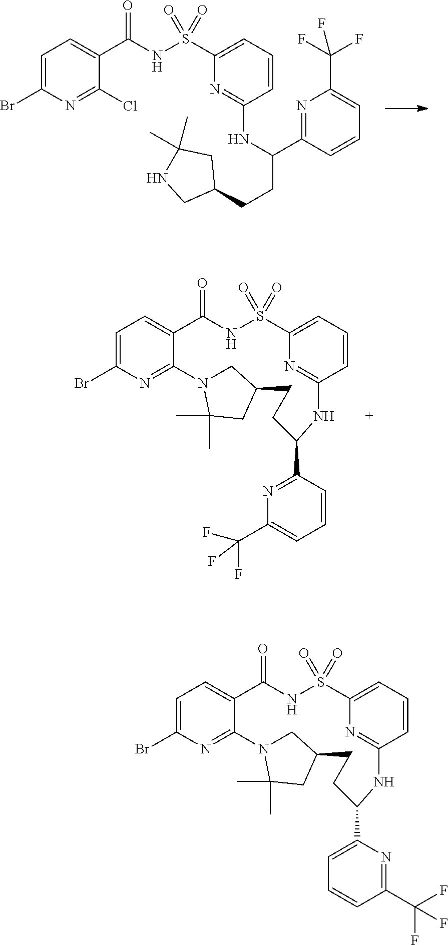 [SMILES: CC1(C)C[C@@H](CCC(Nc2cccc(S(=O)(=O)NC(=O)c3ccc(Br)nc3Cl)n2)c2cccc(C(F)(F)F)n2)CN1 ; CC1(C)C[C@@H]2CC[C@H](c3cccc(C(F)(F)F)n3)Nc3cccc(n3)S(=O)(=O)NC(=O)c3ccc(Br)nc3N1C2 ; CC1(C)C[C@@H]2CC[C@@H](c3cccc(C(F)(F)F)n3)Nc3cccc(n3)S(=O)(=O)NC(=O)c3ccc(Br)nc3N1C2]

A mixture 6-bromo-2-chloro-N-[[6-[[3-[(3R)-5,5-dimethylpyrrolidin-3-yl]-1-[6-(trifluoromethyl)-2-pyridyl]propyl]amino]-2-pyridyl]sulfonyl]pyridine-3-carboxamide (trifluoroacetate salt) (772 mg, 0.7583 mmol) and potassium carbonate (629 mg, 4.551 mmol) in DMA (7.7 mL) was heated at 140° C. for 7 h, cooled by ice bath, diluted with 100 mL of EtOAc and 30 mL of water and then 15 mL of 1 M HCl which produced a mixture with a pH=2. The mixture was partitioned and the organic layer separated. The aqueous layer was extracted with 100 mL of EtOAc. The combined organic extracts were dried (MgSO₄) and evaporated to an oil. The residue was dissolved into DMSO (100 mg/mL) and diluted with 1 volume of DMA. This solution was purified with preparative SFC eluting a gradient of 5 mM NH₃ in methanol to CO₂ (40-70% over 10 min) through a 21.2×250 mm 2-PIC column, 5 μm particle giving as white solids after evaporation of the collected fractions:

First diastereomer to elute: (14S,17R)-8-bromo-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione (101 mg, 21%). ¹H NMR (400 MHz, Chloroform-d) δ 7.80 (t, J=7.9 Hz, 1H), 7.63 (d, J=8.1 Hz, 1H), 7.57 (t, J=9.0 Hz, 1H), 7.52 (d, J=7.9 Hz, 0H), 7.46 (d, J=7.3 Hz, 1H), 6.77 (d, J=7.9 Hz, 1H), 6.71 (d, J=8.4 Hz, 1H), 5.54-5.41 (m, 2H), 3.33 (d, J 9.6 Hz, 1H), 2.97 (d, J=10.6 Hz, 1H), 2.54 (s, 2H), 1.96 (p, J=6.0 Hz, 3H), 1.68 (s, 2H), 1.63 (s, 3H), 1.58 (s, 2H), 1.55 (s, 3H). ESI-MS m/z calc. 638.0923, found 639.2 (M+1)⁺; Retention time: 1.79 minutes (LC method A).

Second diastereomer to elute: (14S,17S)-8-bromo-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione (77 mg, 16%). ¹H NMR (400 MHz, Chloroform-d) δ 7.90 (t, J=7.8 Hz, 1H), 7.85 (d, J=8.1 Hz, 1H), 7.67-7.58 (m, 3H), 7.54 (d, J=8.0 Hz, 1H), 6.99 (d, J=7.9 Hz, 1H), 6.79 (d, J=7.4 Hz, 1H), 6.47 (s, 1H), 5.01 (s, 1H), 3.55 (t, J=8.6 Hz, 1H), 3.23 (s, 1H), 2.34 (d, J=12.9 Hz, 1H), 2.02-1.80 (m, 5H), 1.72 (t, J=11.5 Hz, 1H), 1.60 (s, 3H), 1.53 (s, 3H). ESI-MS m/z calc. 638.0923, found 639.3 (M+1)⁺; Retention time: 1.86 minutes (LC method A).

### Example 59: Preparation of (14S,17R)-8-bromo-17-(4-tert-butylpyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, Compound 55 and (14S,17S)-8-bromo-17-(4-tert-butylpyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione

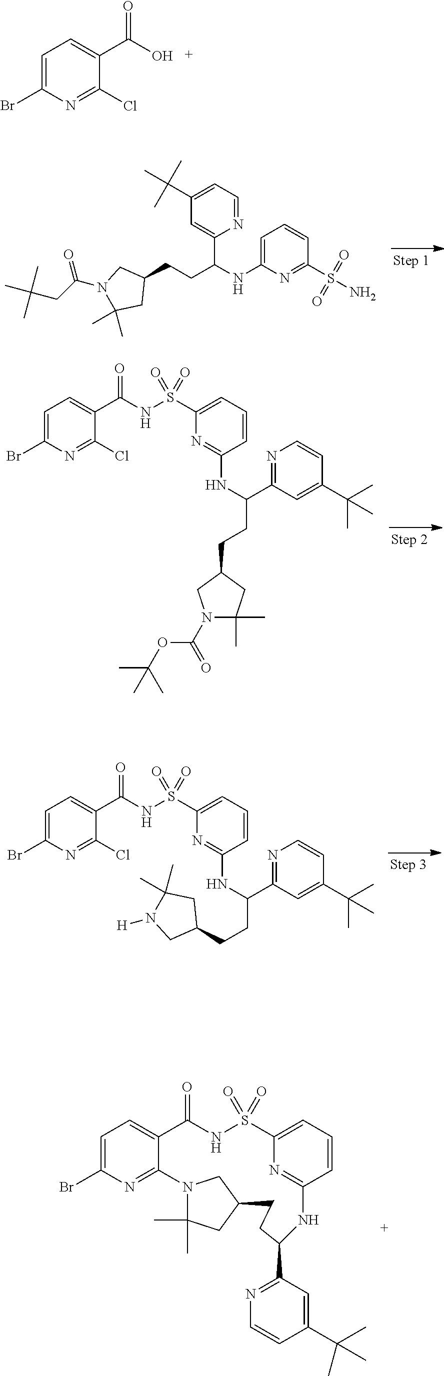

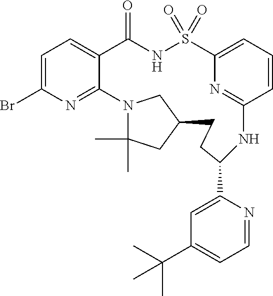 [SMILES: CC(C)(C)c1ccnc([C@@H]2CC[C@@H]3CN(c4nc(Br)ccc4C(=O)NS(=O)(=O)c4cccc(n4)N2)C(C)(C)C3)c1]

### Step 1: tert-Butyl (4S)-4-[3-[[6-[(6-bromo-2-chloro-pyridine-3-carbonyl)sulfamoyl]-2-pyridyl]amino]-3-(4-tert-butyl-2-pyridyl)propyl]-2,2-dimethyl-pyrrolidine-1-carboxylate

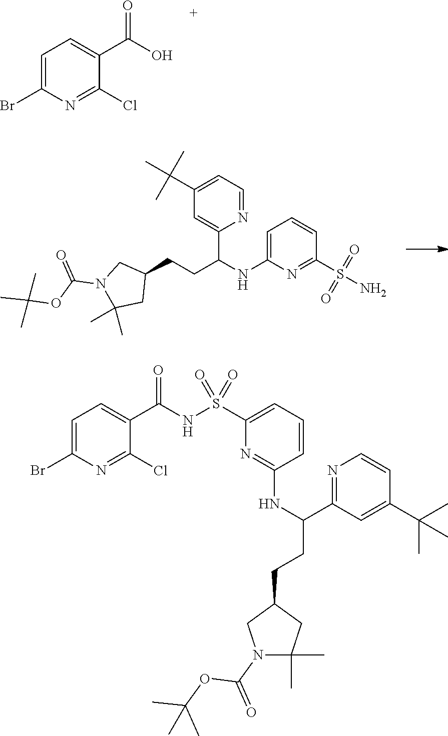 [SMILES: O=C(O)c1ccc(Br)nc1Cl ; CC(C)(C)OC(=O)N1C[C@@H](CCC(Nc2cccc(S(N)(=O)=O)n2)c2cc(C(C)(C)C)ccn2)CC1(C)C ; CC(C)(C)OC(=O)N1C[C@@H](CCC(Nc2cccc(S(=O)(=O)NC(=O)c3ccc(Br)nc3Cl)n2)c2cc(C(C)(C)C)ccn2)CC1(C)C]

A mixture of 6-bromo-2-chloro-pyridine-3-carboxylic acid (510 mg, 2.157 mmol) and CDI (350 mg, 2.159 mmol) was dissolved in THF (3.6 mL) and stirred at 60° C. for 45 min then tert-butyl (4S)-4-[3-(4-tert-butyl-2-pyridyl)-3-[(6-sulfamoyl-2-pyridyl)amino]propyl]-2,2-dimethyl-pyrrolidine-1-carboxylate (587 mg, 1.076 mmol) was added followed by DBU (725 μL, 4.848 mmol) and the resulting mixture was stirred at rt for 2 h. The mixture was diluted with EtOAc and washed with 1 N HCl in water, water, brine, dried (MgSO₄), and concentrated. The residue was purified (flash chromatography: 40 g SiO₂, 10-80% EtOAc in hexanes, 20 min) to provide the desired product: tert-Butyl (4S)-4-[3-[[6-[(6-bromo-2-chloro-pyridine-3-carbonyl)sulfamoyl]-2-pyridyl]amino]-3-(4-tert-butyl-2-pyridyl)propyl]-2,2-dimethyl-pyrrolidine-1-carboxylate (707 mg, 86%). ¹H NMR (400 MHz, Chloroform-d) δ 8.54 (d, J=5.4 Hz, 1H), 7.78 (d, J=8.5 Hz, 1H), 7.58-7.46 (m, 2H), 7.43 (d, J=7.8 Hz, 1H), 7.34 (s, 1H), 7.29 (s, 1H), 6.62 (s, 1H), 5.94-5.81 (m, 1H), 5.12 (d, J=68.4 Hz, 1H), 4.24-4.09 (m, 1H), 3.05 (d, J=11.5 Hz, 0H), 2.85 (t, J=10.8 Hz, 1H), 2.41 (s, 1H), 2.17 (s, 1H), 1.95-1.68 (m, 2H), 1.41 (d, J=4.2 Hz, 9H), 1.35 (s, 5H), 1.33 (s, 4H), 1.32-1.24 (m, 4H). ESI-MS m/z calc. 762.1966, found 764.9 (M+1)⁺; Retention time: 0.64 minutes (LC method D).

### Step 2: 6-Bromo-N-[[6-[[1-(4-tert-butyl-2-pyridyl)-3-[(3R)-5,5-dimethylpyrrolidin-3-yl]propyl]amino]-2-pyridyl]sulfonyl]-2-chloro-pyridine-3-carboxamide

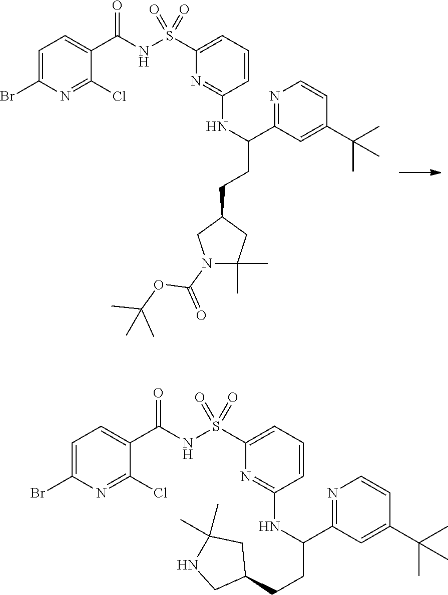 [SMILES: CC(C)(C)OC(=O)N1C[C@@H](CCC(Nc2cccc(S(=O)(=O)NC(=O)c3ccc(Br)nc3Cl)n2)c2cc(C(C)(C)C)ccn2)CC1(C)C ; CC1(C)C[C@@H](CCC(Nc2cccc(S(=O)(=O)NC(=O)c3ccc(Br)nc3Cl)n2)c2cc(C(C)(C)C)ccn2)CN1]

To a solution of tert-butyl (4S)-4-[3-[[6-[(6-bromo-2-chloro-pyridine-3-carbonyl)sulfamoyl]-2-pyridyl]amino]-3-(4-tert-butyl-2-pyridyl)propyl]-2,2-dimethyl-pyrrolidine-1-carboxylate (707 mg, 0.925 mmol) in DCM (5 mL) was added TFA (2.2 mL, 28.56 mmol) and the mixture was stirred at rt for 45 min. The solution was concentrated to dryness under reduced pressure, then co-evaporated with THF (2×20 mL), then dried under high vacuum at rt for 16 h to provide: 6-bromo-N-[[6-[[1-(4-tert-butyl-2-pyridyl)-3-[(3R)-5,5-dimethylpyrrolidin-3-yl]propyl]amino]-2-pyridyl]sulfonyl]-2-chloro-pyridine-3-carboxamide (trifluoroacetate salt) (984 mg, 106%). ESI-MS m/z calc. 662.14417, found 665.0 (M+1)⁺; Retention time: 0.39 minutes (LC method D).

### Step 3: (14S,17R)-8-bromo-17-(4-tert-butylpyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, Compound 55 and (14S,17S)-8-bromo-17-(4-tert-butylpyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione

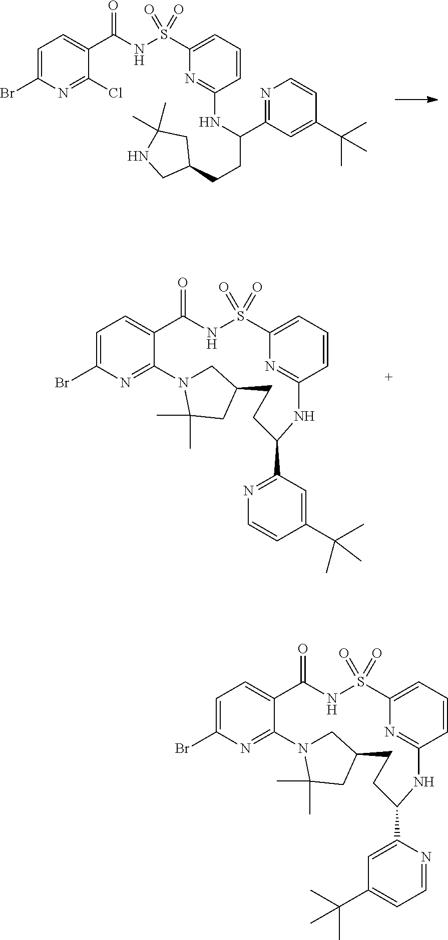 [SMILES: CC1(C)C[C@@H](CCC(Nc2cccc(S(=O)(=O)NC(=O)c3ccc(Br)nc3Cl)n2)c2cc(C(C)(C)C)ccn2)CN1 ; CC(C)(C)c1ccnc([C@H]2CC[C@@H]3CN(c4nc(Br)ccc4C(=O)NS(=O)(=O)c4cccc(n4)N2)C(C)(C)C3)c1 ; CC(C)(C)c1ccnc([C@@H]2CC[C@@H]3CN(c4nc(Br)ccc4C(=O)NS(=O)(=O)c4cccc(n4)N2)C(C)(C)C3)c1]

A mixture of 6-bromo-N-[[6-[[1-(4-tert-butyl-2-pyridyl)-3-[(3R)-5,5-dimethylpyrrolidin-3-yl]propyl]amino]-2-pyridyl]sulfonyl]-2-chloro-pyridine-3-carboxamide (trifluoroacetate salt) (984 mg, 0.978 mmol) and potassium carbonate (811 mg, 5.868 mmol) in DMA (10 mL) was heated at 140° C. for 7 h, cooled by ice bath, diluted with 100 mL of EtOAc and 30 mL of water and then 15 mL of 1 M HCl (2.5×the amount of K₂CO₃) which produced a mixture with a pH 2. The mixture was partitioned and the organic layer separated. The aqueous layer was extracted with 100 mL of EtOAc. The combined organic extracts were dried (MgSO₄) and evaporated to an oil. The residue was dissolved into DMSO (100 mg/mL) and diluted with 1 volume of DMA. This solution was purified with preparative SFC eluting a gradient of 5 mM NH₃ in methanol to CO₂ (40-70% over 10 min) though a 21.2×250 mm 2-PIC column, 5 μm particle giving as a white solid (after evaporation of the collected fractions):

First to elute: (14S,17R)-8-bromo-17-(4-tert-butylpyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (139 mg, 23%). ¹H NMR (400 MHz, Chloroform-d) δ 8.35 (d, J=5.3 Hz, 1H), 7.62 (d, J=7.9 Hz, 1H), 7.54 (t, J=7.8 Hz, 1H), 7.43 (d, J=7.3 Hz, 1H), 7.16 (d, J=5.3 Hz, 1H), 6.76 (d, J=7.9 Hz, 1H), 6.64 (d, J=8.4 Hz, 1H), 5.56 (s, 1H), 5.35 (q, J=7.9 Hz, 1H), 3.47 (s, 1H), 2.56 (s, 1H), 1.98 (dd, J=12.1, 6.8 Hz, 1H), 1.90 (s, 2H), 1.62 (s, 3H), 1.60 (s, 2H), 1.56 (s, 3H), 1.48 (d, J 28.3 Hz, 3H), 1.29 (s, 9H). ESI-MS m/z calc. 626.1675, found 625.3 (M+1)⁺; Retention time: 1.28 minutes (LC method A).

Second to elute: (14S,17S)-8-bromo-17-(4-tert-butylpyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (145 mg, 24%). ¹H NMR (400 MHz, Chloroform-d) δ 8.48 (d, J=5.2 Hz, 1H), 7.84 (s, 1H), 7.62-7.54 (m, 2H), 7.24 (d, J 5.7 Hz, 1H), 6.99 (s, 1H), 6.71 (s, 1H), 6.50 (s, 1H), 4.83 (s, 1H), 3.59 (s, 1H), 3.26 (s, 1H), 2.33 (s, 1H), 1.99 (s, 2H), 1.85 (s, 2H), 1.71 (t, J=11.6 Hz, 1H), 1.59 (s, 3H), 1.52 (s, 3H), 1.46 (d, J=27.1 Hz, 1H), 1.34 (s, 9H), 1.32-1.29 (m, 1H). ESI-MS m/z calc. 626.1675, found 627.3 (M+1)⁺; Retention time: 1.35 minutes (LC method A).

### Example 60: Preparation of benzyl 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]piperidine-1-carboxylate, Compound 255 (diastereomer 1), and Compound 254 (diastereomer 2)

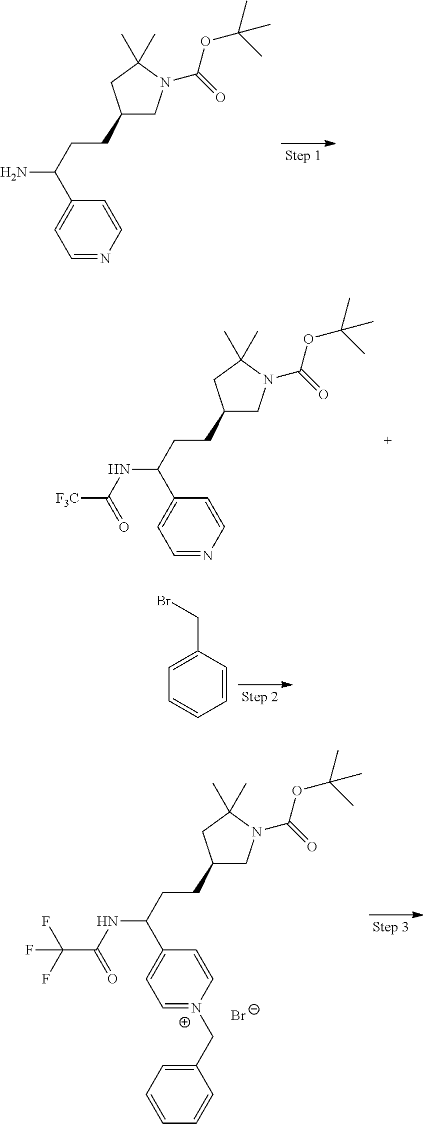

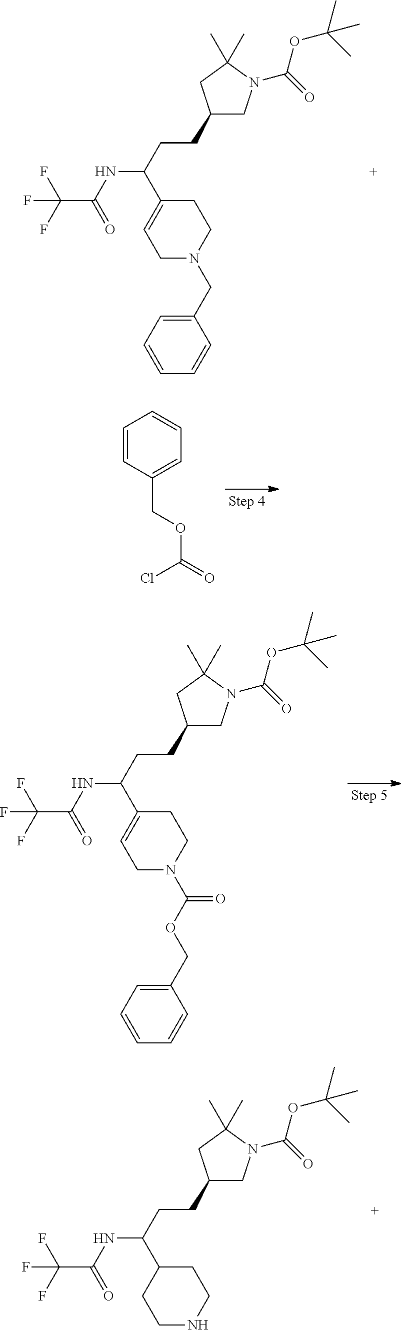

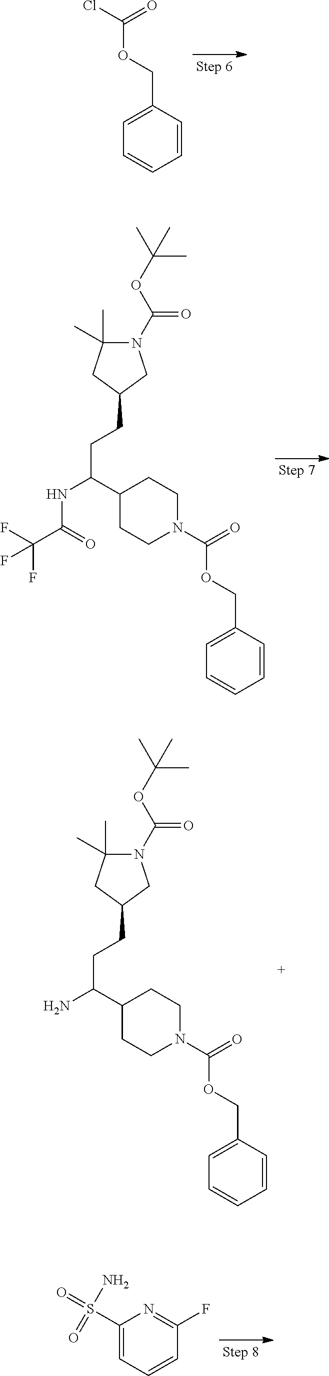 [SMILES: O=C(Cl)OCc1ccccc1 ; CC(C)(C)OC(=O)N1C[C@@H](CCC(NC(=O)C(F)(F)F)C2CCN(C(=O)OCc3ccccc3)CC2)CC1(C)C ; CC(C)(C)OC(=O)N1C[C@@H](CCC(N)C2CCN(C(=O)OCc3ccccc3)CC2)CC1(C)C ; NS(=O)(=O)c1cccc(F)n1]

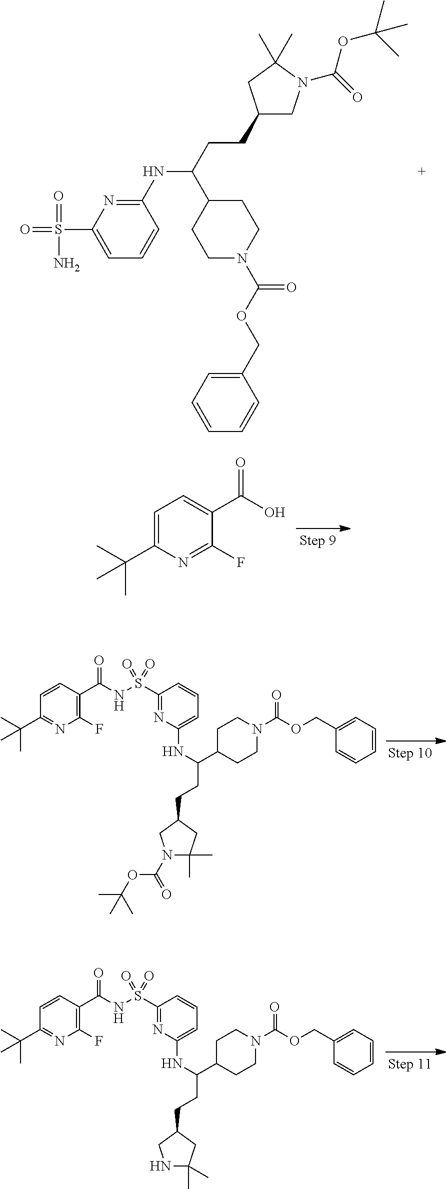

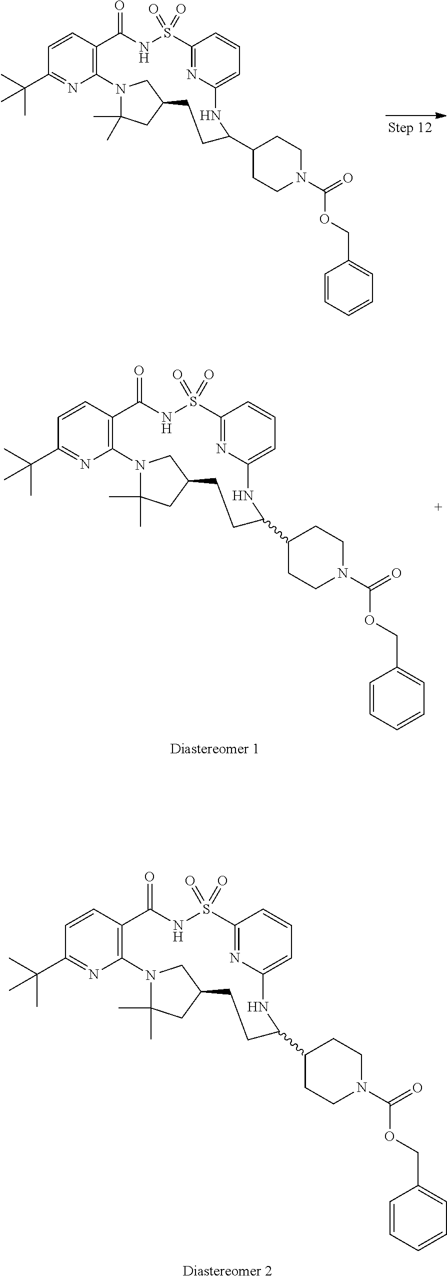 [SMILES: CC(C)(C)c1ccc2c(n1)N1C[C@@H](CCC(C3CCN(C(=O)OCc4ccccc4)CC3)Nc3cccc(n3)S(=O)(=O)NC2=O)CC1(C)C ; CC(C)(C)c1ccc2c(n1)N1C[C@@H](CCC(C3CCN(C(=O)OCc4ccccc4)CC3)Nc3cccc(n3)S(=O)(=O)NC2=O)CC1(C)C ; CC(C)(C)c1ccc2c(n1)N1C[C@@H](CCC(C3CCN(C(=O)OCc4ccccc4)CC3)Nc3cccc(n3)S(=O)(=O)NC2=O)CC1(C)C]

### Step 1: tert-Butyl (4S)-2,2-dimethyl-4-[3-(4-pyridyl)-3-[(2,2,2-trifluoroacetyl)amino]propyl]pyrrolidine-1-carboxylate

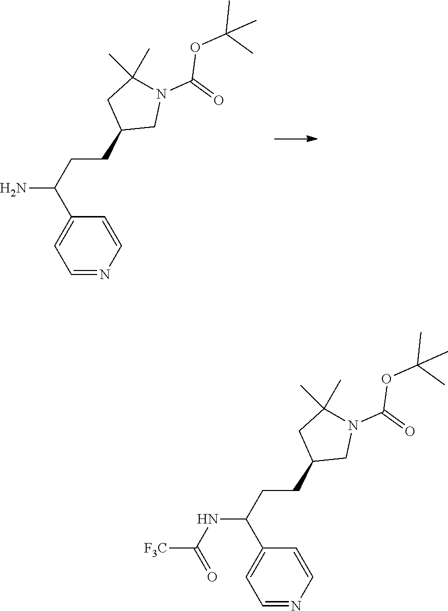 [SMILES: CC(C)(C)OC(=O)N1C[C@@H](CCC(N)c2ccncc2)CC1(C)C ; CC(C)(C)OC(=O)N1C[C@@H](CCC(NC(=O)C(F)(F)F)c2ccncc2)CC1(C)C]

tert-Butyl (4S)-4-[3-amino-3-(4-pyridyl)propyl]-2,2-dimethyl-pyrrolidine-1-carboxylate (1 g, 2.6989 mmol) was dissolved in DCM (20 mL) and cooled in an ice water bath. TEA (0.5 mL, 3.5873 mmol) was added, followed by (2,2,2-trifluoroacetyl) 2,2,2-trifluoroacetate (0.45 mL, 3.1288 mmol). The mixture was stirred in the cooling bath under nitrogen for 1 h. Sodium bicarbonate (saturated aqueous 20 mL) was added. The layers were separated and the aqueous layer was washed with brine, dried over anhydrous Na₂SO₄, filtered and concentrated to afford tert-butyl (4S)-2,2-dimethyl-4-[3-(4-pyridyl)-3-[(2,2,2-trifluoroacetyl)amino]propyl]pyrrolidine-1-carboxylate as a colorless oil (910 mg, 75%). ESI-MS m/z calc. 429.2239, found 430.6 (M+1)⁺; Retention time: 2.83 minutes (LC method B).

### Step 2: tert-Butyl (4S)-4-[3-(1-benzylpyridin-1-ium-4-yl)-3-[(2,2,2-trifluoroacetyl)amino]propyl]-2,2-dimethyl-pyrrolidine-1-carboxylate; bromide

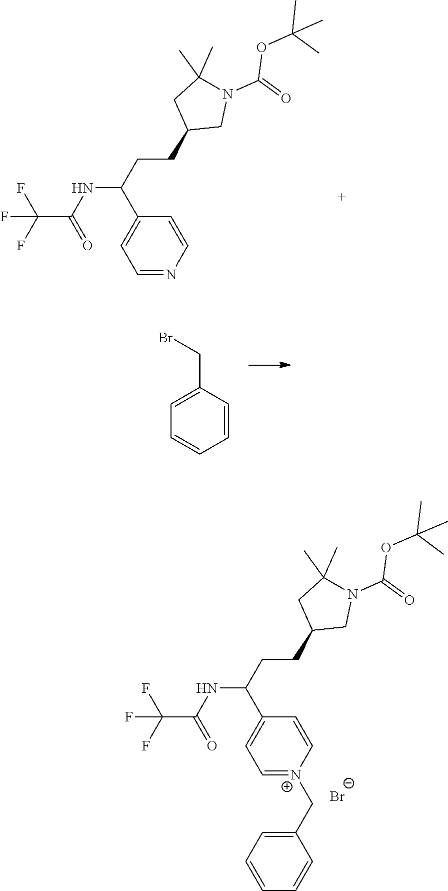 [SMILES: CC(C)(C)OC(=O)N1C[C@@H](CCC(NC(=O)C(F)(F)F)c2ccncc2)CC1(C)C ; BrCc1ccccc1 ; CC(C)(C)OC(=O)N1C[C@@H](CCC(NC(=O)C(F)(F)F)c2cc[n+](Cc3ccccc3)cc2)CC1(C)C ; [Br-]]

tert-Butyl (4S)-2,2-dimethyl-4-[3-(4-pyridyl)-3-[(2,2,2-trifluoroacetyl)amino]propyl]pyrrolidine-1-carboxylate (910 mg, 2.0129 mmol) was dissolved in CH₃CN (5 mL). Bromomethylbenzene (0.24 mL, 1.9802 mmol) was added. The mixture was heated at 50° C. for 12 h. It was then concentrated to afford tert-butyl (4S)-4-[3-(1-benzylpyridin-1-ium-4-yl)-3-[(2,2,2-trifluoroacetyl)amino]propyl]-2,2-dimethyl-pyrrolidine-1-carboxylate; bromide as a pale yellow foam (1.2 g, 94%). ESI-MS m/z calc. 599.197, found 520.7 (M+1)⁺; Retention time: 3.47 minutes (LC method B).

### Step 3: tert-Butyl (4S)-4-[3-(1-benzyl-3,6-dihydro-2H-pyridin-4-yl)-3-[(2,2,2-trifluoroacetyl)amino]propyl]-2,2-dimethyl-pyrrolidine-1-carboxylate

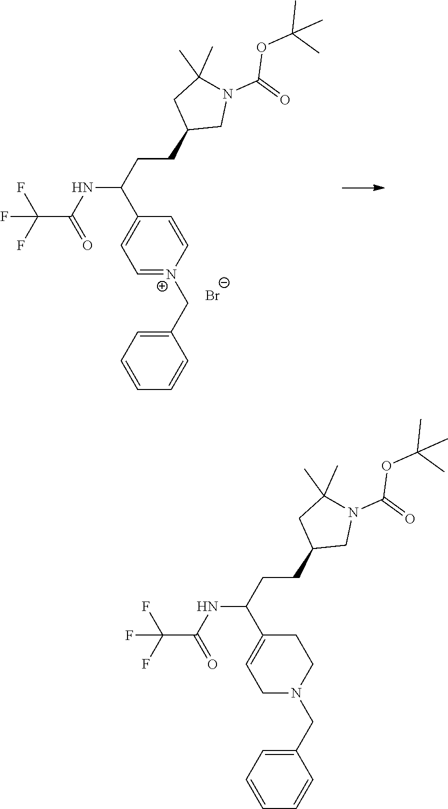 [SMILES: CC(C)(C)OC(=O)N1C[C@@H](CCC(NC(=O)C(F)(F)F)c2cc[n+](Cc3ccccc3)cc2)CC1(C)C ; [Br-] ; CC(C)(C)OC(=O)N1C[C@@H](CCC(NC(=O)C(F)(F)F)C2=CCN(Cc3ccccc3)CC2)CC1(C)C]

tert-Butyl (4S)-4-[3-(1-benzylpyridin-1-ium-4-yl)-3-[(2,2,2-trifluoroacetyl)amino]propyl]-2,2-dimethyl-pyrrolidine-1-carboxylate; bromide (1.2 g, 1.8984 mmol) was dissolved in MeOH (10 mL). The mixture was cooled in ice water bath. NaBH₄ (152 mg, 3.9374 mmol) was added in three portions. The mixture was stirred at rt for 1 h. NH₄Cl (saturated aqueous 1.0 mL) was added and the mixture was concentrated to remove volatiles. The residue was then partitioned between water and DCM (20 mL each). The layers were separated and the aqueous layer was extracted with more DCM (10 mL). The combined DCM layer was dried over anhydrous Na₂SO₄, filtered and concentrated to afford tert-butyl (4S)-4-[3-(1-benzyl-3,6-dihydro-2H-pyridin-4-yl)-3-[(2,2,2-trifluoroacetyl)amino]propyl]-2,2-dimethyl-pyrrolidine-1-carboxylate (1.1 g, 105%). ESI-MS m/z calc. 523.3022, found 524.6 (M+1)⁺; Retention time: 3.42 minutes (LC method B).

### Step 4: Benzyl 4-[3-[(3S)-1-tert-butoxycarbonyl-5,5-dimethyl-pyrrolidin-3-yl]-1-[(2,2,2-trifluoroacetyl)amino]propyl]-3,6-dihydro-2H-pyridine-1-carboxylate

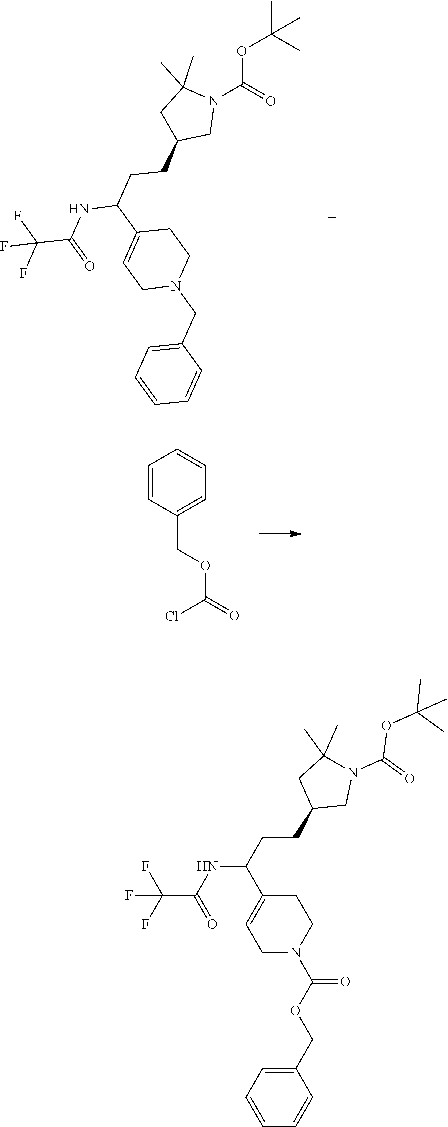 [SMILES: CC(C)(C)OC(=O)N1C[C@@H](CCC(NC(=O)C(F)(F)F)C2=CCN(Cc3ccccc3)CC2)CC1(C)C ; O=C(Cl)OCc1ccccc1 ; CC(C)(C)OC(=O)N1C[C@@H](CCC(NC(=O)C(F)(F)F)C2=CCN(C(=O)OCc3ccccc3)CC2)CC1(C)C]

tert-Butyl (4S)-4-[3-(1-benzyl-3,6-dihydro-2H-pyridin-4-yl)-3-[(2,2,2-trifluoroacetyl)amino]propyl]-2,2-dimethyl-pyrrolidine-1-carboxylate (1.1 g, 1.9957 mmol) was dissolved in DCM (30 mL) at rt. Potassium bicarbonate (900 mg, 8.9897 mmol) was added, followed by benzyl chloroformate (737.91 mg, 0.65 mL, 4.1093 mmol). The mixture was stirred in a 40° C. oil bath for 1 h. Water (20 mL) was added and the layers were separated. The DCM layer was dried over anhydrous Na₂SO₄, filtered and concentrated. The residue was purified by silica gel chromatography (40 g column), using 0-30% EtOAc in hexanes to afford benzyl 4-[3-[(3S)-1-tert-butoxycarbonyl-5,5-dimethyl-pyrrolidin-3-yl]-1-[(2,2,2-trifluoroacetyl)amino]propyl]-3,6-dihydro-2H-pyridine-1-carboxylate as a slightly yellow oil (1.01 g, 85%). ESI-MS m/z calc. 567.292, found 568.7 (M+1)⁺; Retention time: 4.33 minutes (LC method B).

### Step 5: tert-Butyl (4S)-2,2-dimethyl-4-[3-(4-piperidyl)-3-[(2,2,2-trifluoroacetyl)amino]propyl]pyrrolidine-1-carboxylate

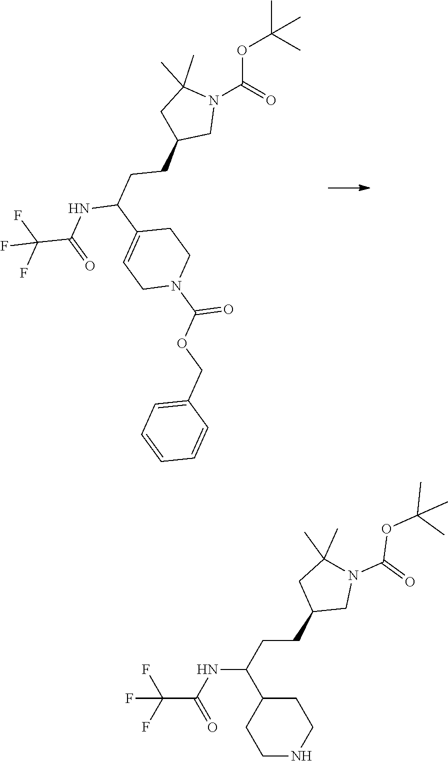 [SMILES: CC(C)(C)OC(=O)N1C[C@@H](CCC(NC(=O)C(F)(F)F)C2=CCN(C(=O)OCc3ccccc3)CC2)CC1(C)C ; CC(C)(C)OC(=O)N1C[C@@H](CCC(NC(=O)C(F)(F)F)C2CCNCC2)CC1(C)C]

Benzyl 4-[3-[(3S)-1-tert-butoxycarbonyl-5,5-dimethyl-pyrrolidin-3-yl]-1-[(2,2,2-trifluoroacetyl)amino]propyl]-3,6-dihydro-2H-pyridine-1-carboxylate (500 mg, 0.8368 mmol) was dissolved in a solvent mixture of MeOH (20 mL) and EtOAc (5 mL). The mixture was purged with nitrogen. Palladium on carbon (10% w:w, 100 mg, 0.0940 mmol) was added in one portion. The mixture was placed under a Parr shaker under 60 psi of hydrogen for 2 h. It was then purged with nitrogen and filtered through a celite pad, washing with MeOH (˜20 mL). The combined filtrate was concentrated to afford tert-butyl (4S)-2,2-dimethyl-4-[3-(4-piperidyl)-3-[(2,2,2-trifluoroacetyl)amino]propyl]pyrrolidine-1-carboxylate (370 mg, 96%) ESI-MS m/z calc. 435.2709, found 436.7 (M+1)⁺; Retention time: 3.04 minutes (LC method B).

### Step 6: Benzyl 4-[3-[(3S)-1-tert-butoxycarbonyl-5,5-dimethyl-pyrrolidin-3-yl]-1-[(2,2,2-trifluoroacetyl)amino]propyl]piperidine-1-carboxylate

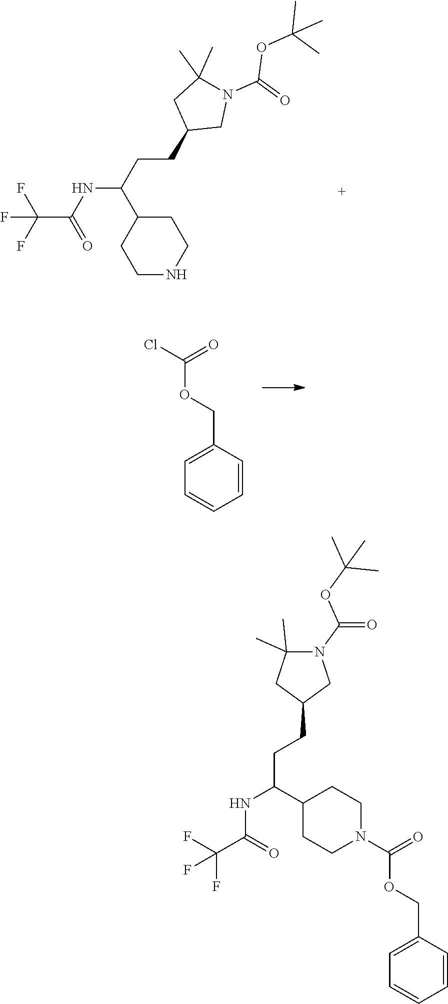 [SMILES: CC(C)(C)OC(=O)N1C[C@@H](CCC(NC(=O)C(F)(F)F)C2CCNCC2)CC1(C)C ; O=C(Cl)OCc1ccccc1 ; CC(C)(C)OC(=O)N1C[C@@H](CCC(NC(=O)C(F)(F)F)C2CCN(C(=O)OCc3ccccc3)CC2)CC1(C)C]

tert-Butyl (4S)-2,2-dimethyl-4-[3-(4-piperidyl)-3-[(2,2,2-trifluoroacetyl)amino]propyl]pyrrolidine-1-carboxylate (370 mg, 0.8071 mmol) was dissolved in DCM (10 mL). TEA (0.15 mL, 1.0762 mmol) was added. The mixture was cooled in ice water bath. Benzyl chloroformate (0.15 mL, 0.9483 mmol) was then added dropwise. The mixture was stirred at this temperature for 30 min. Saturated aqueous sodium bicarbonate (20 mL) was added. The layers were separated and the aqueous layer was extracted with more DCM (15 mL). The combined organics was dried over anhydrous Na₂S 04, filtered and concentrated to afford benzyl 4-[3-[(3S)-1-tert-butoxycarbonyl-5,5-dimethyl-pyrrolidin-3-yl]-1-[(2,2,2-trifluoroacetyl)amino]propyl]piperidine-1-carboxylate (415 mg, 86%). ESI-MS m/z calc. 569.3077, found 570.6 (M+1)⁺; Retention time: 4.37 minutes (LC method B).

### Step 7: Benzyl 4-[1-amino-3-[(35)-1-tert-butoxycarbonyl-5,5-dimethyl-pyrrolidin-3-yl]propyl]piperidine-1-carboxylate

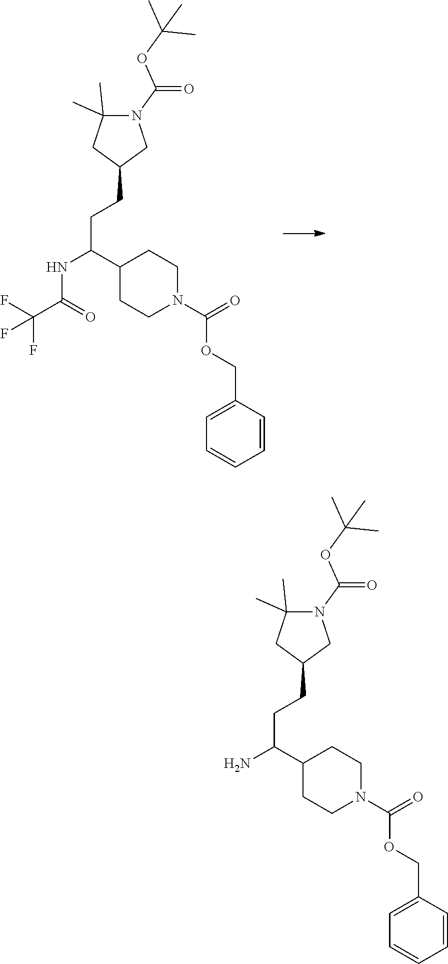 [SMILES: CC(C)(C)OC(=O)N1C[C@@H](CCC(NC(=O)C(F)(F)F)C2CCN(C(=O)OCc3ccccc3)CC2)CC1(C)C ; CC(C)(C)OC(=O)N1C[C@@H](CCC(N)C2CCN(C(=O)OCc3ccccc3)CC2)CC1(C)C]

Benzyl 4-[3-[(3S)-1-tert-butoxycarbonyl-5,5-dimethyl-pyrrolidin-3-yl]-1-[(2,2,2-trifluoroacetyl)amino]propyl]piperidine-1-carboxylate (1 g, 1.6677 mmol) was dissolved in THF (10 mL). LiOH (400 mg, 16.369 mmol) in water (5 mL) was added, followed by MeOH (5 mL). The mixture was heated for 15 h in a 50° C. oil bath. It was cooled to rt and concentrated to remove most volatiles. The residue was partitioned between brine and DCM (30 mL each). The DCM layer was dried over anhydrous Na₂SO₄, filtered and concentrated to afford benzyl 4-[1-amino-3-[(3S)-1-tert-butoxycarbonyl-5,5-dimethyl-pyrrolidin-3-yl]propyl]piperidine-1-carboxylate as a white foam. (800 mg, 96%). ESI-MS m/z calc. 473.3254, found 474.6 (M+1)⁺; Retention time: 3.31 minutes (LC method B).

### Step 8: Benzyl 4-[3-[(3S)-1-tert-butoxycarbonyl-5,5-dimethyl-pyrrolidin-3-yl]-1-[(6-sulfamoyl-2-pyridyl)amino]propyl]piperidine-1-carboxylate

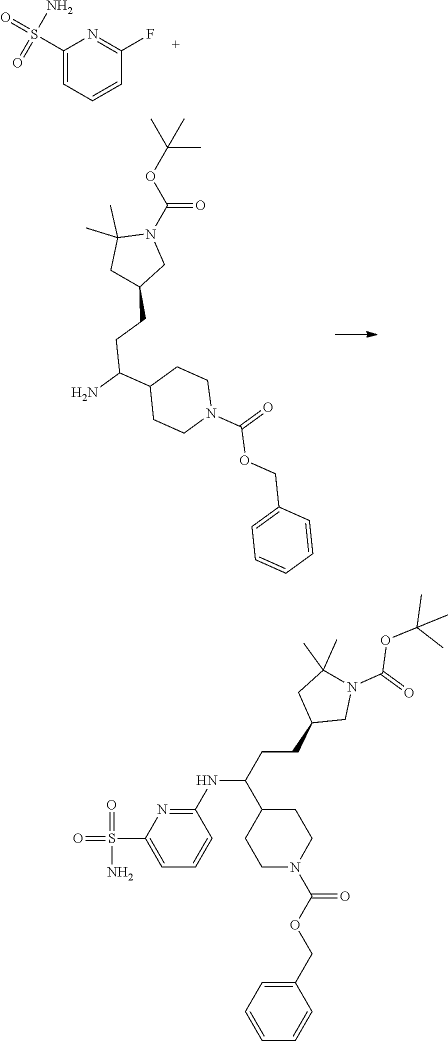

Benzyl 4-[1-amino-3-[(3S)-1-tert-butoxycarbonyl-5,5-dimethyl-pyrrolidin-3-yl]propyl]piperidine-1-carboxylate (800 mg, 1.6046 mmol) was dissolved in DMSO (1.5 mL). 6-Fluoropyridine-2-sulfonamide (338 mg, 1.9294 mmol) was added, followed by Na₂CO₃ (512 mg, 4.8307 mmol). The mixture was heated under nitrogen balloon in a 110° C. oil bath for 22 h. It was then cooled to rt and partitioned between EtOAc (30 mL) and water (30 mL). The layers were separated and the aqueous layer was extracted with more water (20 mL). The organic layer was dried over anhydrous Na₂SO₄, filtered and concentrated. The residue was purified by silica gel chromatography (40 g column), using 0-35% EtOAc in hexanes to afford benzyl 4-[3-[(3S)-1-tert-butoxycarbonyl-5,5-dimethyl-pyrrolidin-3-yl]-1-[(6-sulfamoyl-2-pyridyl)amino]propyl]piperidine-1-carboxylate as a pale yellow foam. (506 mg, 46%). ¹H NMR (500 MHz, DMSO-d₆) δ 7.48 (d, J=7.8 Hz, 1H), 7.41-7.26 (m, 5H), 7.04 (s, 2H), 6.91 (d, J=7.2 Hz, 1H), 6.78 (d, J=8.9 Hz, 1H), 6.63 (d, J=8.5 Hz, 1H), 5.05 (s, 2H), 4.10-3.96 (m, 2H), 3.60-3.44 (m, 1H), 2.84-2.62 (m, 3H), 2.07 (d, J=10.6 Hz, 1H), 1.86 (td, J=12.8, 12.3, 6.2 Hz, 1H), 1.67 (q, J=18.0, 16.4 Hz, 3H), 1.52 (s, 1H), 1.44-1.27 (m, 15H), 1.22 (d, J=3.2 Hz, 3H), 1.18 (q, J=6.7, 6.3 Hz, 1H), 1.15-1.04 (m, 1H). ESI-MS m/z calc. 629.3247, found 630.8 (M+1)⁺; Retention time: 2.98 minutes (LC method H).

### Step 9: Benzyl 4-[3-[(3S)-1-tert-butoxycarbonyl-5,5-dimethyl-pyrrolidin-3-yl]-1-[[6-[(6-tert-butyl-2-fluoro-pyridine-3-carbonyl)sulfamoyl]-2-pyridyl]amino]propyl]piperidine-1-carboxylate

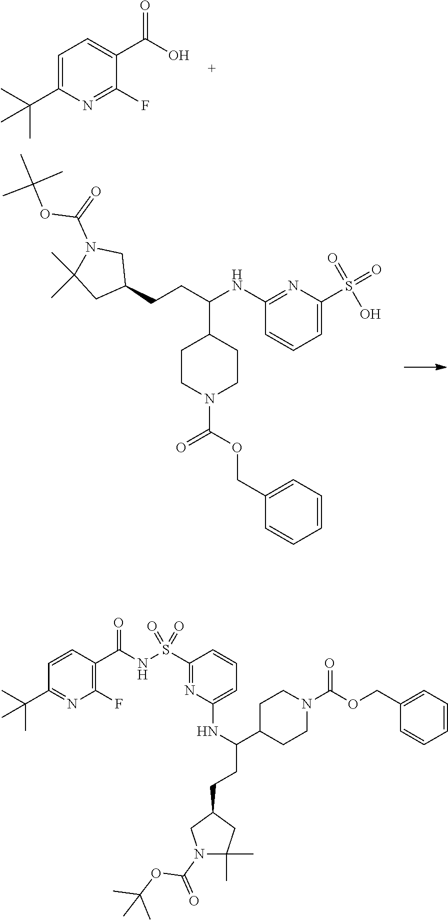

To a solution of 6-tert-butyl-2-fluoro-pyridine-3-carboxylic acid (127 mg, 0.6440 mmol) in THF (4.5 mL) was added CDI (110 mg, 0.6784 mmol) and the mixture was stirred at rt for 20 h. Then benzyl 4-[3-[(3S)-1-tert-butoxycarbonyl-5,5-dimethyl-pyrrolidin-3-yl]-1-[(6-sulfamoyl-2-pyridyl)amino]propyl]piperidine-1-carboxylate (225 mg, 0.3573 mmol) was added followed by DBU (225 μL, 1.505 mmol) and the resulting mixture was stirred for 18 h at rt. The reaction was diluted with ethyl acetate and washed with a sat aq sodium bicarbonate solution. The organic layer was further washed with 10% citric acid solution followed by brine. The organics were separated, dried over sodium sulfate, and evaporated and then purified on silica gel chromatography (40 gram column) using a gradient from 100% hexanes to 100% ethyl acetate to afford a white solid benzyl 4-[3-[(3 S)-1-tert-butoxycarbonyl-5,5-dimethyl-pyrrolidin-3-yl]-1-[[6-[(6-tert-butyl-2-fluoro-pyridine-3-carbonyl)sulfamoyl]-2-pyridyl]amino]propyl]piperidine-1-carboxylate (213 mg, 63%). ESI-MS m/z calc. 808.39935, found 809.2 (M+1)⁺; Retention time: 2.44 minutes (LC method A).

### Step 10: Benzyl 4-[1-[[6-[(6-tert-butyl-2-fluoro-pyridine-3-carbonyl)sulfamoyl]-2-pyridyl]amino]-3-[(3S)-5,5-dimethylpyrrolidin-3-yl]propyl]piperidine-1-carboxylate

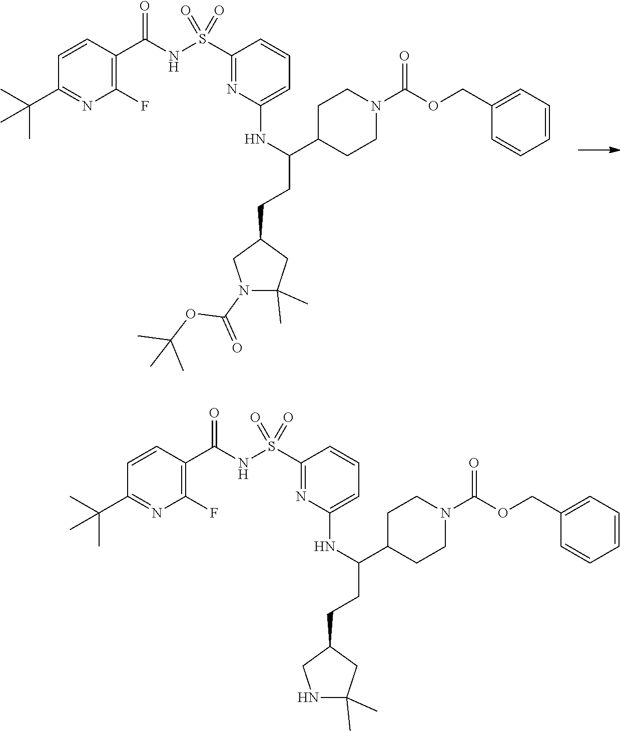 [SMILES: CC(C)(C)OC(=O)N1C[C@@H](CCC(Nc2cccc(S(=O)(=O)NC(=O)c3ccc(C(C)(C)C)nc3F)n2)C2CCN(C(=O)OCc3ccccc3)CC2)CC1(C)C ; CC1(C)C[C@H](CCC(Nc2cccc(S(=O)(=O)NC(=O)c3ccc(C(C)(C)C)nc3F)n2)C2CCN(C(=O)OCc3ccccc3)CC2)CN1]

Benzyl 4-[3-[(3S)-1-tert-butoxycarbonyl-5,5-dimethyl-pyrrolidin-3-yl]-1-[[6-[(6-tert-butyl-2-fluoro-pyridine-3-carbonyl)sulfamoyl]-2-pyridyl]amino]propyl]piperidine-1-carboxylate (213 mg, 0.2633 mmol) was dissolved in DCM (5.0 mL) and to the mixture was added TFA (550 μL, 7.139 mmol) and the reaction was stirred at room temperature. After 14 h, the mixture was evaporated to dryness, then diluted with diethyl ether (30 mL×2), and reconcentrated. The material was then placed on the high vacuum pump for 2 h to afford benzyl 4-[1-[[6-[(6-tert-butyl-2-fluoro-pyridine-3-carbonyl)sulfamoyl]-2-pyridyl]amino]-3-[(3S)-5,5-dimethylpyrrolidin-3-yl]propyl]piperidine-1-carboxylate (trifluoroacetate salt) as a pale yellow oil (270 mg, 100%). ESI-MS m/z calc. 708.3469, found 709.2 (M+1)⁺; Retention time: 1.64 minutes (LC method A).

### Step 11: benzyl 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]piperidine-1-carboxylate

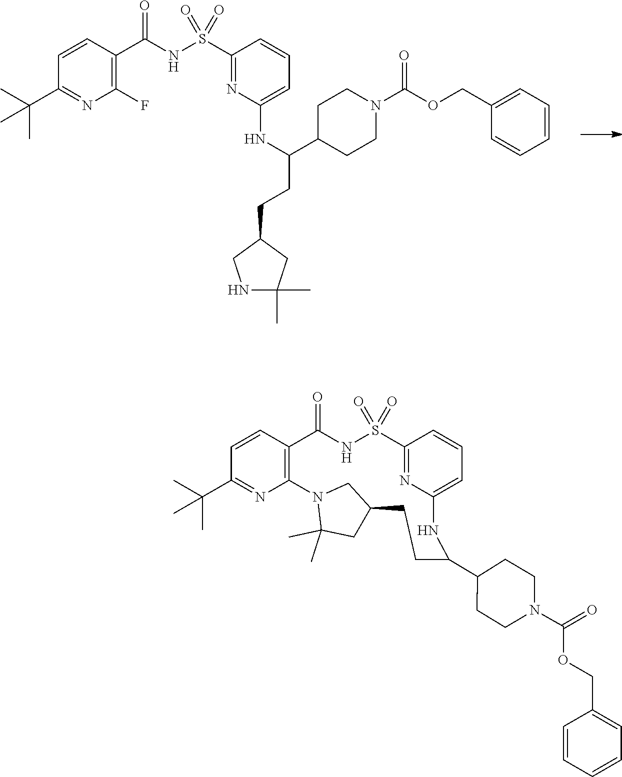 [SMILES: CC1(C)C[C@H](CCC(Nc2cccc(S(=O)(=O)NC(=O)c3ccc(C(C)(C)C)nc3F)n2)C2CCN(C(=O)OCc3ccccc3)CC2)CN1 ; CC(C)(C)c1ccc2c(n1)N1C[C@@H](CCC(C3CCN(C(=O)OCc4ccccc4)CC3)Nc3cccc(n3)S(=O)(=O)NC2=O)CC1(C)C]

To a solution of benzyl 4-[1-[[6-[(6-tert-butyl-2-fluoro-pyridine-3-carbonyl)sulfamoyl]-2-pyridyl]amino]-3-[(3S)-5,5-dimethylpyrrolidin-3-yl]propyl]piperidine-1-carboxylate (Trifluoroacetate salt) (216 mg, 0.2100 mmol) in NMP (5 mL) was added potassium carbonate (304 mg, 2.200 mmol). The mixture was purged with nitrogen for 5 min. The mixture was heated at 140° C. for 16 h. The mixture was cooled to room temperature, diluted with 1N HCl and extracted with EtOAc (1×). The organic phase was washed with brine (1×), dried (sodium sulfate), filtered and concentrated to a brown oil which was filtered and purified using a reverse phase HPLC-MS method using a dual gradient run of 50-99% mobile phase B over 15.0 minutes (mobile phase A=H₂O (5 mM HCl, mobile phase B=acetonitrile) giving as an off-white solid, benzyl 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]piperidine-1-carboxylate (84 mg, 58%). ESI-MS m/z calc. 688.3407, found 689.2 (M+1)⁺; Retention time: 0.77 minutes (LC method I).

### Step 12: benzyl 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]piperidine-1-carboxylate, Compound 255 (diastereomer 1), and benzyl 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]piperidine-1-carboxylate, Compound 254 (diastereomer 2)

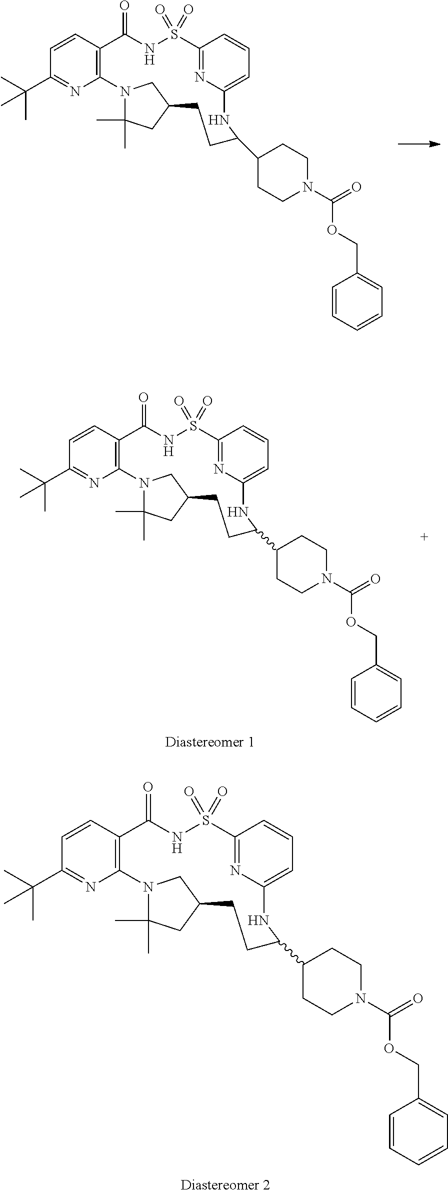 [SMILES: CC(C)(C)c1ccc2c(n1)N1C[C@@H](CCC(C3CCN(C(=O)OCc4ccccc4)CC3)Nc3cccc(n3)S(=O)(=O)NC2=O)CC1(C)C ; CC(C)(C)c1ccc2c(n1)N1C[C@@H](CCC(C3CCN(C(=O)OCc4ccccc4)CC3)Nc3cccc(n3)S(=O)(=O)NC2=O)CC1(C)C ; CC(C)(C)c1ccc2c(n1)N1C[C@@H](CCC(C3CCN(C(=O)OCc4ccccc4)CC3)Nc3cccc(n3)S(=O)(=O)NC2=O)CC1(C)C]

Benzyl 4-[(14S)-8-tert-butyl-12,12-dimethyl-2,2,4-tri oxo-2λ⁶-thia-3,9,11,18,23-pentaazatetracyclo[17.3.1.111,14.05,10]tetracosa-1(23),5(10),6,8,19,21-hexaen-17-yl]piperidine-1-carboxylate (diastereomeric mixture, 20 mg, 0.02903 mmol) was subjected to reverse phase preparative phase HPLC-MS using a a dual gradient run of 50-99% mobile phase B over 30.0 minutes (mobile phase A=H₂O (5 mM HCl), mobile phase B=CH₃CN) to give too separated isomers:

First isomer to elute, diastereomer 1: Benzyl 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]piperidine-1-carboxylate (7.7 mg, 75%). ¹H NMR (499 MHz, Chloroform-d) δ 9.47 (s, 1H), 7.83 (s, 1H), 7.55 (t, J=7.8 Hz, 1H), 7.46 (d, J=7.2 Hz, 1H), 7.39-7.28 (m, 6H), 6.75 (s, 1H), 6.55 (d, J=8.3 Hz, 1H), 5.08 (s, 2H), 4.23 (d, J=43.7 Hz, 4H), 3.15 (s, 1H), 2.88 (s, 1H), 2.72 (s, 2H), 2.49 (s, 1H), 1.97 (d, J=10.9 Hz, 1H), 1.70 (d, J=11.4 Hz, 2H), 1.66-1.62 (m, 5H), 1.58 (s, 6H), 1.44 (d, J=6.3 Hz, 2H), 1.31 (s, 9H). ESI-MS m/z calc. 688.3407, found 689.3 (M+1)⁺; Retention time: 1.95 minutes (LC method G).

Second isomer to elute, diastereomer 2: Benzyl 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]piperidine-1-carboxylate (8.2 mg, 82%). ¹H NMR (499 MHz, Chloroform-d) δ 12.12 (s, 1H), 8.13 (d, J=8.2 Hz, 1H), 7.65 (d, J=7.3 Hz, 1H), 7.59 (d, J=7.8 Hz, 1H), 7.37 (d, J=4.4 Hz, 4H), 7.35-7.30 (m, 2H), 7.04 (d, J=8.1 Hz, 1H), 6.60 (d, J=8.3 Hz, 1H), 5.14 (s, 2H), 4.67 (s, 1H), 4.29 (s, 2H), 3.40 (s, 1H), 3.30 (dd, J=10.1, 4.4 Hz, 1H), 3.16 (t, J=9.6 Hz, 1H), 2.78 (s, 2H), 2.39 (s, 1H), 2.28 (s, 1H), 2.02 (dd, J=12.0, 7.7 Hz, 1H), 1.80 (s, 2H), 1.75-1.64 (m, 4H), 1.61 (s, 3H), 1.56 (s, 2H), 1.52 (s, 3H), 1.44-1.35 (m, 2H), 1.32 (s, 9H). ESI-MS m/z calc. 688.3407, found 689.3 (M+1)⁺; Retention time: 1.95 minutes (LC method G).

### Example 61: Preparation of (14S)-8-tert-butyl-12,12-dimethyl-17-(1-methylpiperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 241 (diastereomer 1), and (14S)-8-tert-butyl-12,12-dimethyl-17-(1-methylpiperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 240 (diastereomer 2)

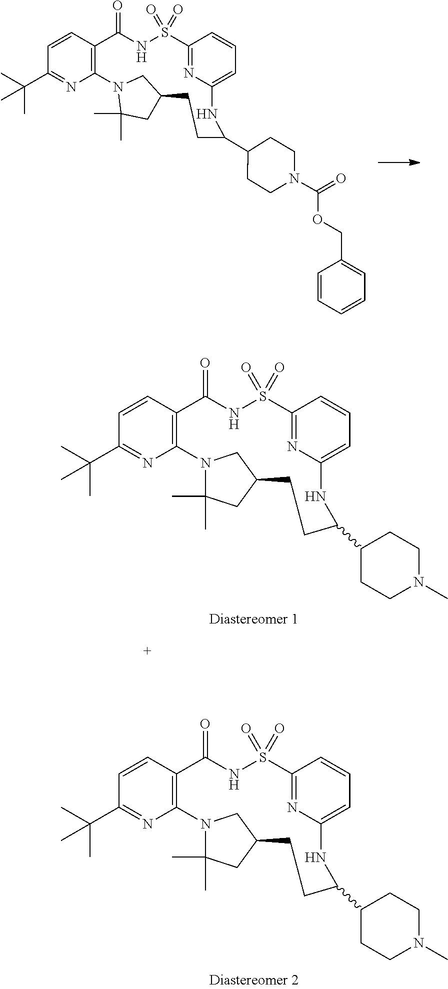

To a solution of benzyl 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]piperidine-1-carboxylate (58.3 mg, 0.08463 mmol) in methanol (2 mL) was added formaldehyde (21.63 μL of 37% w/v, 0.2665 mmol) followed by palladium on carbon (10% w/w, 9 mg, 0.008463 mmol) and the mixture was stirred with nitrogen bubbling through the mixture for 2 minutes. Hydrogen gas was then bubbled through the solution for 2 min then the mixture was stirred under 1 atm of hydrogen for 16 h. Added 10% palladium on carbon (9 mg, 0.008463 mmol) and continued stirring under 1 atm of hydrogen gas for 4 h then filtered over celite, concentrated and purified using a reverse phase HPLC-MS method using a Luna C₁₈ column (75×30 mm, 5 μm particle size) sold by Phenomenex (pn: 00C-4252-U0-AX), and a dual gradient run from 1-99% mobile phase B over 15.0 minutes (mobile phase A=H₂O (5 mM HCl), mobile phase B=acetonitrile, flow rate=50 mL/min, injection volume=950 μL and column temperature=25° C.) giving two isomers as white solids:

First to elute, diastereomer 1: (14S)-8-tert-Butyl-12,12-dimethyl-17-(1-methylpiperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (13.5 mg, 50%). ESI-MS m/z calc. 568.3196, found 569.3 (M+1)⁺; Retention time: 1.71 minutes (LC method Q).

Second to elute, diastereomer 2: (14S)-8-tert-Butyl-12,12-dimethyl-17-(1-methylpiperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (10.8 mg, 40%) ESI-MS m/z calc. 568.3196, found 569.2 (M+1)⁺; Retention time: 1.84 minutes (LC method Q).

### Example 62: Preparation of (14S)-8-tert-butyl-12,12-dimethyl-17-(piperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 249 (diastereomer 1), and Compound 248 (diastereomer 2)

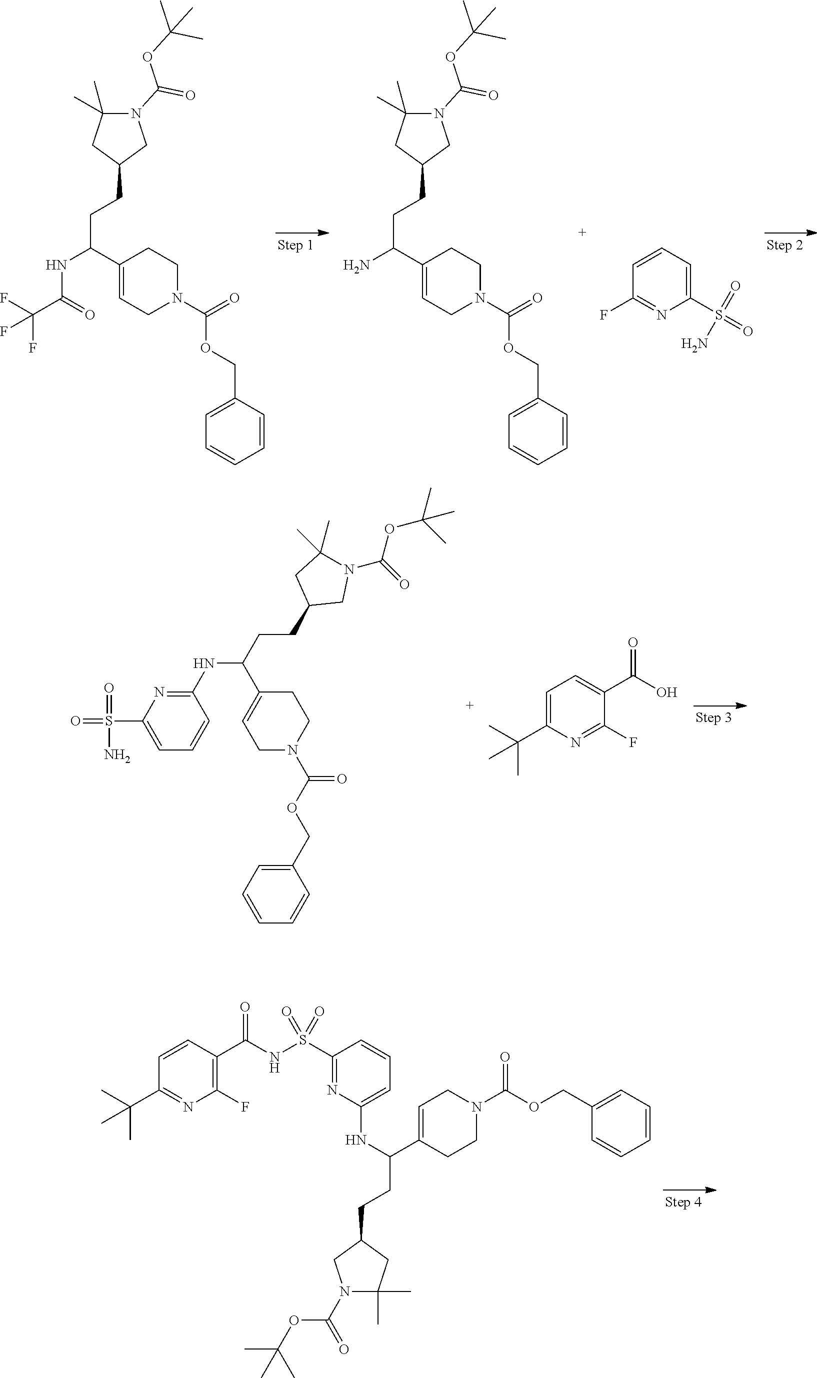 [SMILES: CC(C)(C)OC(=O)N1C[C@@H](CCC(NC(=O)C(F)(F)F)C2=CCN(C(=O)OCc3ccccc3)CC2)CC1(C)C ; CC(C)(C)OC(=O)N1C[C@@H](CCC(N)C2=CCN(C(=O)OCc3ccccc3)CC2)CC1(C)C ; NS(=O)(=O)c1cccc(F)n1 ; CC(C)(C)OC(=O)N1C[C@@H](CCC(Nc2cccc(S(N)(=O)=O)n2)C2=CCN(C(=O)OCc3ccccc3)CC2)CC1(C)C ; CC(C)(C)c1ccc(C(=O)O)c(F)n1 ; CC(C)(C)OC(=O)N1C[C@@H](CCC(Nc2cccc(S(=O)(=O)NC(=O)c3ccc(C(C)(C)C)nc3F)n2)C2=CCN(C(=O)OCc3ccccc3)CC2)CC1(C)C]

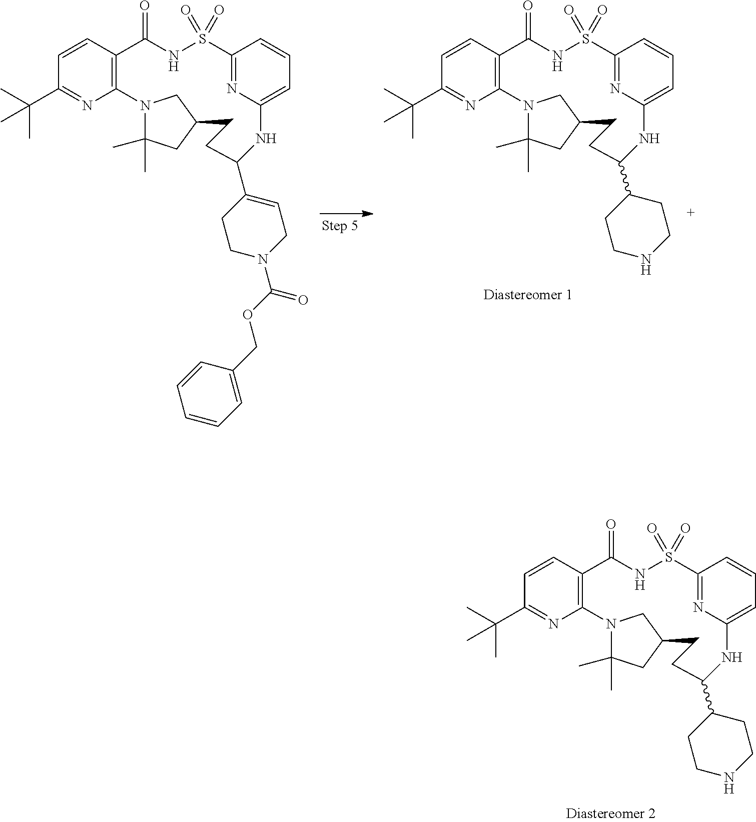 [SMILES: CC(C)(C)c1ccc2c(n1)N1C[C@@H](CCC(C3=CCN(C(=O)OCc4ccccc4)CC3)Nc3cccc(n3)S(=O)(=O)NC2=O)CC1(C)C ; CC(C)(C)c1ccc2c(n1)N1C[C@@H](CCC(C3CCNCC3)Nc3cccc(n3)S(=O)(=O)NC2=O)CC1(C)C ; CC(C)(C)c1ccc2c(n1)N1C[C@@H](CCC(C3CCNCC3)Nc3cccc(n3)S(=O)(=O)NC2=O)CC1(C)C]

### Step 1: Benzyl 4-[1-amino-3-[(3S)-1-tert-butoxycarbonyl-5,5-dimethyl-pyrrolidin-3-yl]propyl]-3,6-dihydro-2H-pyridine-1-carboxylate

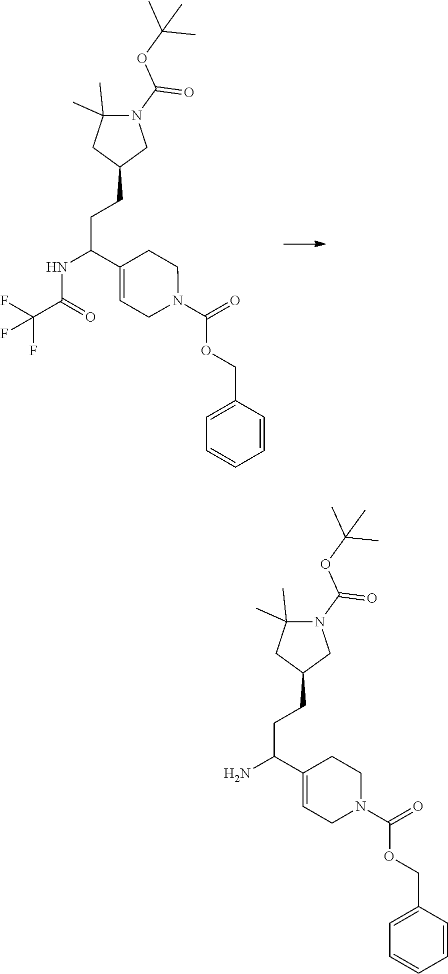 [SMILES: CC(C)(C)OC(=O)N1C[C@@H](CCC(NC(=O)C(F)(F)F)C2=CCN(C(=O)OCc3ccccc3)CC2)CC1(C)C ; CC(C)(C)OC(=O)N1C[C@@H](CCC(N)C2=CCN(C(=O)OCc3ccccc3)CC2)CC1(C)C]

Benzyl 4-[3-[(3S)-1-tert-butoxycarbonyl-5,5-dimethyl-pyrrolidin-3-yl]-1-[(2,2,2-trifluoroacetyl)amino]propyl]-3,6-dihydro-2H-pyridine-1-carboxylate (520 mg, 0.8703 mmol) was mixed in THF (5 mL) and MeOH (3 mL) at rt. LiOH (209 mg, 8.5526 mmol) in water (3 mL) was added. The mixture was heated in a 50° C. oil bath for 3 h. It was then cooled to rt and concentrated. The residue was partitioned between water and DCM (20 mL each). The DCM layer was dried over anhydrous Na₂SO₄, filtered and dried under high vacuum overnight to afford crude benzyl 4-[1-amino-3-[(3S)-1-tert-butoxycarbonyl-5,5-dimethyl-pyrrolidin-3-yl]propyl]-3,6-dihydro-2H-pyridine-1-carboxylate as a yellowish foam (400 mg, 93%). ESI-MS m/z calc. 471.3097, found 472.7 (M+1)⁺; Retention time: 3.06 minutes (LC method B).

### Step 2: Benzyl 4-[3-[(3S)-1-tert-butoxycarbonyl-5,5-dimethyl-pyrrolidin-3-yl]-1-[(6-sulfamoyl-2-pyridyl)amino]propyl]-3,6-dihydro-2H-pyridine-1-carboxylate

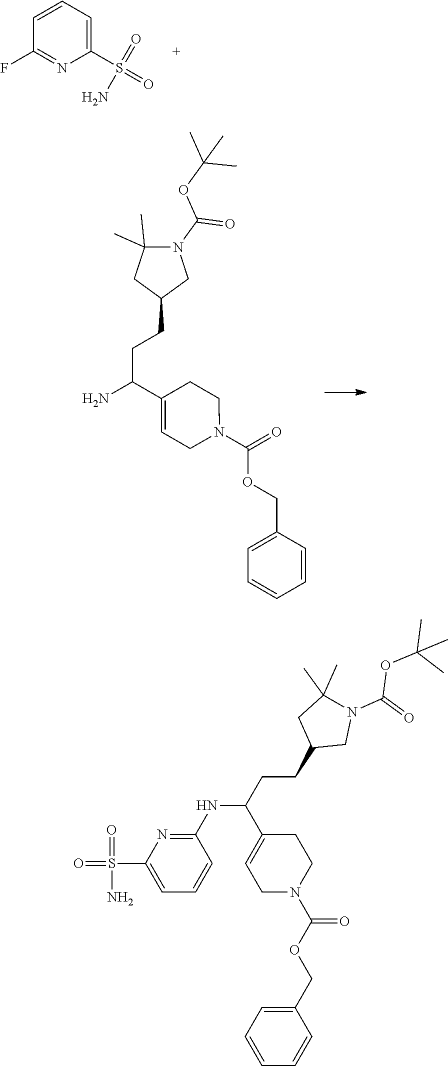

Benzyl 4-[1-amino-3-[(3S)-1-tert-butoxycarbonyl-5,5-dimethyl-pyrrolidin-3-yl]propyl]-3,6-dihydro-2H-pyridine-1-carboxylate (400 mg, 0.8057 mmol) was dissolved in DMSO (2 mL). 6-Fluoropyridine-2-sulfonamide (184 mg, 1.0503 mmol) was added, followed by Na₂CO₃ (258 mg, 2.4342 mmol). The mixture was heated in a 110° C. oil bath under nitrogen balloon for 20 h. It was then cooled to rt and diluted with water (15 mL) and EtOAc (25 mL). The layers were separated and the aqueous layer was extracted with more EtOAc (10 mL). The combined organics was dried over anhydrous Na₂SO₄, filtered and concentrated. The residue was purified by silica gel flash chromatography (40 g column) using 5-80% EtOAc in hexanes to afford benzyl 4-[3-[(3S)-1-tert-butoxy carbonyl-5,5-dimethyl-pyrrolidin-3-yl]-1-[(6-sulfamoyl-2-pyridyl)amino]propyl]-3,6-dihydro-2H-pyridine-1-carboxylate as a pale yellow foam (220 mg, 41%). ¹H NMR (500 MHz, DMSO-d₆) δ 7.53 (t, J=7.8 Hz, 1H), 7.36 (d, J=6.6 Hz, 4H), 7.32 (td, J=5.9, 2.4 Hz, 1H), 7.08 (dd, J=8.4, 3.6 Hz, 1H), 7.05 (s, 2H), 6.96 (d, J=7.2 Hz, 1H), 6.62 (d, J=8.5 Hz, 1H), 5.08 (s, 2H), 4.45 (s, 1H), 3.86 (d, J=19.1 Hz, 2H), 3.60-3.41 (m, 3H), 2.78 (p, J=10.4, 10.0 Hz, 1H), 2.08 (s, 3H), 1.88 (s, 1H), 1.55 (s, 2H), 1.46-1.30 (m, 14H), 1.24 (d, J=2.1 Hz, 3H). ESI-MS m/z calc. 627.3091, found 628.4 (M+1)⁺; Retention time: 2.93 minutes (LC method H).

### Step 3: benzyl 4-[3-[(3S)-1-tert-butoxycarbonyl-5,5-dimethyl-pyrrolidin-3-yl]-1-[[6-[(6-tert-butyl-2-fluoro-pyridine-3-carbonyl)sulfamoyl]-2-pyridyl]amino]propyl]-3,6-dihydro-2H-pyridine-1-carboxylate

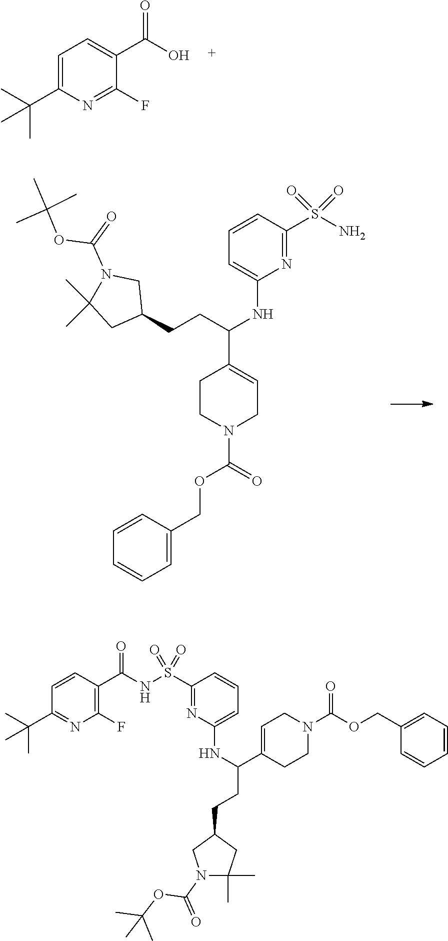

To a solution of 6-tert-butyl-2-fluoro-pyridine-3-carboxylic acid (125 mg, 0.6339 mmol) in THF (4.5 mL) was added CDI (110 mg, 0.6784 mmol) and the mixture was stirred at rt for 20 h. Then benzyl 4-[3-[(3S)-1-tert-butoxycarbonyl-5,5-dimethyl-pyrrolidin-3-yl]-1-[(6-sulfamoyl-2-pyridyl)amino]propyl]-3,6-dihydro-2H-pyridine-1-carboxylate (218 mg, 0.3472 mmol) was added followed by DBU (250 μL, 1.672 mmol) and the resulting mixture was stirred for 18 h at rt. The reaction was diluted with ethyl acetate and washed with a saturated aqueous sodium bicarbonate solution. The organic layer was further washed with 10% citric acid solution followed by brine. The organics were separated, dried over sodium sulfate, and evaporated. The crude material was then purified by reverse-phase preparative chromatography utilizing a C₁₈ column and a 30-99% over 15 min gradient of cetonitrile in water (+5 mmolar HCl) to afford the diastereomeric pair benzyl 4-[3-[(3S)-1-tert-butoxycarbonyl-5,5-dimethyl-pyrrolidin-3-yl]-1-[[6-[(6-tert-butyl-2-fluoro-pyridine-3-carbonyl)sulfamoyl]-2-pyridyl]amino]propyl]-3,6-dihydro-2H-pyridine-1-carboxylate as a white solid (180 mg, 64%). ESI-MS m/z calc. 806.38367, found 807.2 (M+1)⁺; Retention time: 2.4 minutes (LC method A).

### Step 4: Benzyl 4-[(14S)-8-tert-butyl-12,12-dimethyl-2,2,4-trioxo-2λ⁶-thia-3,9,11,18,23-pentaazatetracyclo[17.3.1.111,14.05,10]tetratosa-1(23),5(10),6,8,19,21-hexaen-17-yl]-1,2,3,6-tetrahydropyridine-1-carboxylate

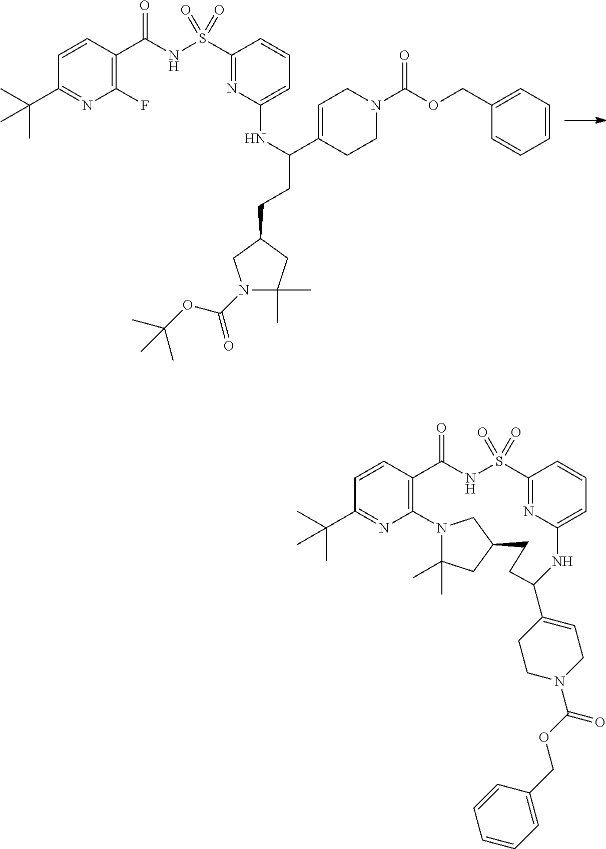 [SMILES: CC(C)(C)OC(=O)N1C[C@@H](CCC(Nc2cccc(S(=O)(=O)NC(=O)c3ccc(C(C)(C)C)nc3F)n2)C2=CCN(C(=O)OCc3ccccc3)CC2)CC1(C)C ; CC(C)(C)c1ccc2c(n1)N1C[C@@H](CCC(C3=CCN(C(=O)OCc4ccccc4)CC3)Nc3cccc(n3)S(=O)(=O)NC2=O)CC1(C)C]

Stage 1: Benzyl 4-[3-[(3S)-1-tert-butoxycarbonyl-5,5-dimethyl-pyrrolidin-3-yl]-1-[[6-[(6-tert-butyl-2-fluoro-pyridine-3-carbonyl)sulfamoyl]-2-pyridyl]amino]propyl]-3,6-dihydro-2H-pyridine-1-carboxylate (180 mg, 0.2231 mmol) was dissolved in DCM (5.5 mL) and to the mixture was added TFA (500 μL, 6.490 mmol). The reaction was stirred at room temperature. After 1 hr, the mixture was evaporated to dryness, then diluted with ether (30 mL×2), and reconcentrated. The material was then placed on the high vacuum pump for 2 h to afford benzyl 4-[1-[[6-[(6-tert-butyl-2-fluoro-pyridine-3-carbonyl)sulfamoyl]-2-pyridyl]amino]-3-[(3S)-5,5-dimethylpyrrolidin-3-yl]propyl]-3,6-dihydro-2H-pyridine-1-carboxylate (trifluoroacetate salt) as an off-white solid.

Stage 2: The intermediate from Step 1 and K₂CO₃ (350 mg, 2.532 mmol), 3 Å molecular sieves and NMP (7.5 mL) were combined in a vial, purged with nitrogen, capped, heated to 155° C. and stirred for 18 h. The reaction was cooled to room temperature and was diluted with ethyl acetate and water. The organic layer was extracted (2×) and was further washed with 10% citric acid solution followed by brine. The organics were separated, dried over sodium sulfate, evaporated to a light brown oil. The residue was purified separately by reverse-phase preparative chromatography utilizing a C₁₈ column and a 30-99% gradient over 15 min of acetonitrile in water (+5 mmolar HCl) to afford the product as a white solid: benzyl 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-1,2,3,6-tetrahydropyridine-1-carboxylate (137 mg, 89%). ESI-MS m/z calc. 686.325, found 687.3 (M+1)⁺; Retention time: 2.3 minutes (LC method A).

### Step 5: (14S)-8-tert-butyl-12,12-dimethyl-17-(piperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 249 (diastereomer 1), and (14S)-8-tert-butyl-12,12-dimethyl-17-(piperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 248 (diastereomer 2)

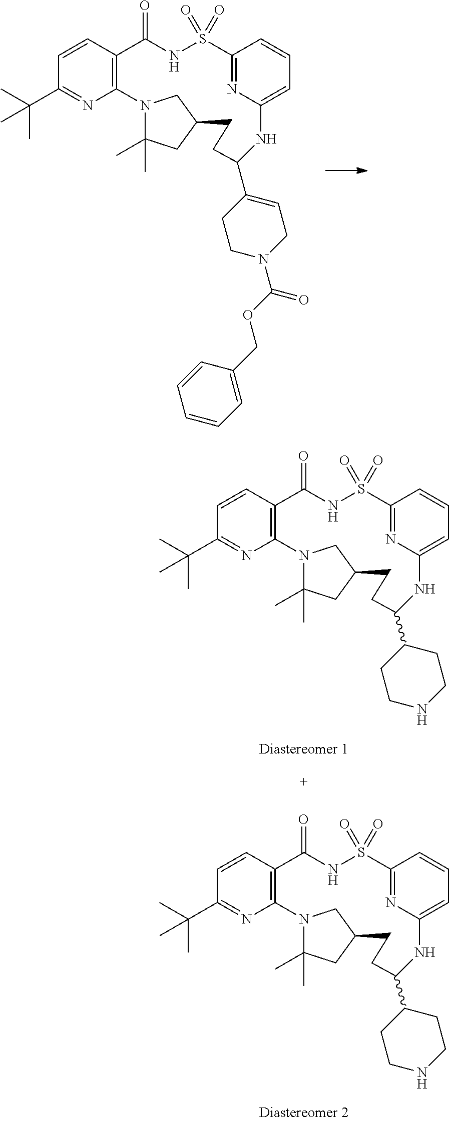 [SMILES: CC(C)(C)c1ccc2c(n1)N1C[C@@H](CCC(C3=CCN(C(=O)OCc4ccccc4)CC3)Nc3cccc(n3)S(=O)(=O)NC2=O)CC1(C)C ; CC(C)(C)c1ccc2c(n1)N1C[C@@H](CCC(C3CCNCC3)Nc3cccc(n3)S(=O)(=O)NC2=O)CC1(C)C ; CC(C)(C)c1ccc2c(n1)N1C[C@@H](CCC(C3CCNCC3)Nc3cccc(n3)S(=O)(=O)NC2=O)CC1(C)C]

To a nitrogen purged 250 mL round bottom flask benzyl 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-1,2,3,6-tetrahydropyridine-1-carboxylate (137 mg, 0.1995 mmol) was dissolved in MeOH (7 mL). To the nitrogen purged reaction solution was added palladium on carbon (10% w:w, 16 mg, 0.015 mmol) and a balloon containing hydrogen gas was attached. Hydrogen gas was purged through the solutions for 1 min prior to stirring under hydrogen balloons for 14 h at rt. crude material was filtered through celite and concentrated under a stream of nitrogen to give a viscous residue. This mixture was diluted with DMSO and was then purified by reverse-phase preparative chromatography utilizing a C₁₈ column and a 1-70% gradient over 30 min of acetonitrile in water (+5 mM HCl) to afford the separate isomers as white solids:

Diastereomer 1: (14S)-8-tert-Butyl-12,12-dimethyl-17-(piperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (34 mg, 58%). ESI-MS m/z calc. 554.3039, found 555.2 (M+1)⁺; Retention time: 1.45 minutes (LC method A).

Diastereomer 2: (14S)-8-tert-Butyl-12,12-dimethyl-17-(piperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (29 mg, 49%). ESI-MS m/z calc. 554.3039, found 555.2 (M+1)⁺; Retention time: 1.61 minutes (LC method A).

### Example 63: Preparation of (14S)-8-tert-Butyl-17-{1-[2-(dimethylamino) acetyl]piperidin-4-yl}-12,12-dimethyl-2λ⁶-thia-3,9,11,18,23-pentaazatetracyclo [17.3.1.111,14.05,10]tetracosa-1(23),5(10),6,8,19,21-hexaene-2,2,4-trione, Compound 236

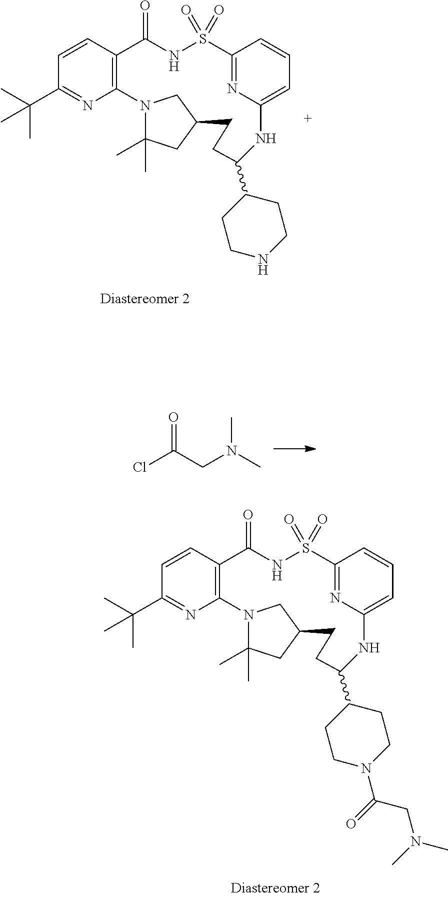

To a solution of (14S)-8-tert-butyl-12,12-dimethyl-17-(piperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (Compound 248 (diastereomer 2), 15 mg, 0.02537 mmol) in DMF (650 μL) was added K₂CO₃ (15 mg, 0.1085 mmol) followed by 2-(dimethylamino)acetyl chloride (5.0 mg, 0.04113 mmol). The reaction was stirred for 20 h, then the mixture was quenched with 0.1 mL concentrated HCl, diluted with DMF, filtered, and purified by reverse-phase preparative chromatography utilizing a C₁₈ column and a 15-75% over 15 min gradient of acetonitrile in water (+5 mM HCl) to afford (14S)-8-tert-butyl-17-{1-[2-(dimethylamino)acetyl]piperidin-4-yl}-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (11.7 mg, 68%). ¹H NMR (500 MHz, DMSO-d₆) δ 12.42 (d, J=13.1 Hz, 1H), 9.70 (d, J=15.9 Hz, 1H), 7.62 (dd, J=7.9, 2.1 Hz, 1H), 7.56 (dd, J=8.5, 7.1 Hz, 1H), 7.10 (d, J=8.8 Hz, 1H), 7.03 (dd, J=7.1, 1.3 Hz, 1H), 6.77 (dd, J=8.4, 2.0 Hz, 1H), 6.64 (d, J=7.9 Hz, 1H), 4.43-4.18 (m, 3H), 3.59 (dd, J 39.2, 13.3 Hz, 1H), 3.11-3.02 (m, 1H), 2.94 (dt, J=24.5, 12.4 Hz, 1H), 2.78 (td, J=5.3, 2.6 Hz, 6H), 2.67-2.53 (m, 2H), 2.07 (d, J=8.4 Hz, 1H), 1.87-1.76 (m, 2H), 1.65 (s, 2H), 1.63 (s, 1H), 1.61 (s, 3H), 1.54 (d, J=10.5 Hz, 2H), 1.51 (s, 1H), 1.48 (s, 3H), 1.36-1.29 (m, 1H), 1.27 (s, 9H), 1.20 (d, J 18.6 Hz, 1H), 1.12 (dd, J 33.7, 9.2 Hz, 1H). ESI-MS m/z calc. 639.3567, found 640.2 (M+1)⁺; Retention time: 1.69 minutes as a white solid (LC method A).

### Example 64: Preparation of (14S)-17-(1-Acetylpiperidin-4-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 228

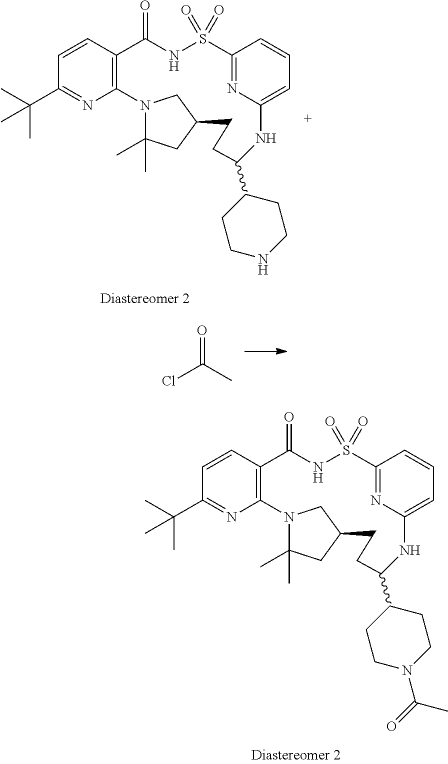

To a solution of (14S)-8-tert-butyl-12,12-dimethyl-17-(piperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (Compound 248 (diastereomer 2), 19.7 mg, 0.03332 mmol) in DMF (1000 μL) was added K₂CO₃ (20 mg, 0.1447 mmol) followed by acetyl chloride (2.5 μL, 0.03516 mmol) in 254 DMF. The reaction was stirred for 20 h. Then the mixture was quenched with 0.1 mL concentrated HCl, diluted with DMF, filtered, and purified by reverse-phase preparative chromatography utilizing a C₁₈ column and a 20-80% gradient over 15 min of acetonitrile in water (+5 mM HCl) to afford (14S)-17-(1-acetylpiperidin-4-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione as a white solid (12.8 mg, 64%). ¹H NMR (500 MHz, DMSO-d₆) δ 12.43 (s, 1H), 7.61 (dd, J=8.0, 2.9 Hz, 1H), 7.56 (dd, J=8.5, 7.2 Hz, 1H), 7.03 (d, J=7.9 Hz, 1H), 6.97 (d, J=9.0 Hz, 1H), 6.73 (d, J=8.5 Hz, 1H), 6.64 (d, J=8.0 Hz, 1H), 4.45-4.31 (m, 2H), 3.97 (s, 1H), 3.79 (dd, J=23.4, 13.6 Hz, 1H), 3.10-2.98 (m, 1H), 2.90 (dd, J=21.9, 11.8 Hz, 1H), 2.64 (t, J=10.5 Hz, 1H), 2.41 (dt, J=33.2, 12.4 Hz, 1H), 2.07 (d, J=12.7 Hz, 1H), 1.93 (d, J=5.2 Hz, 3H), 1.84-1.73 (m, 2H), 1.64 (s, 1H), 1.62 (s, 3H), 1.59-1.54 (m, 2H), 1.52 (d, J=12.4 Hz, 1H), 1.48 (s, 4H), 1.27 (s, 9H), 1.23 (d, J=13.7 Hz, 1H), 1.17-1.09 (m, 1H), 1.09-0.92 (m, 1H). ESI-MS m/z calc. 596.31445, found 597.2 (M+1)⁺; Retention time: 1.97 minutes (LC method A).

### Example 65: Preparation of (14S)-8-tert-butyl-12,12-dimethyl-17-[1-(3,3,3-trifluoropropyl)piperidin-4-yl]-2λ⁶-thia-3,9,11,18,23-pentaazatetracyclo [17.3.1.111,14.05,10]tetracosa-1(23),5(10),6,8,19,21-hexaene-2,2,4-trione, Compound 238

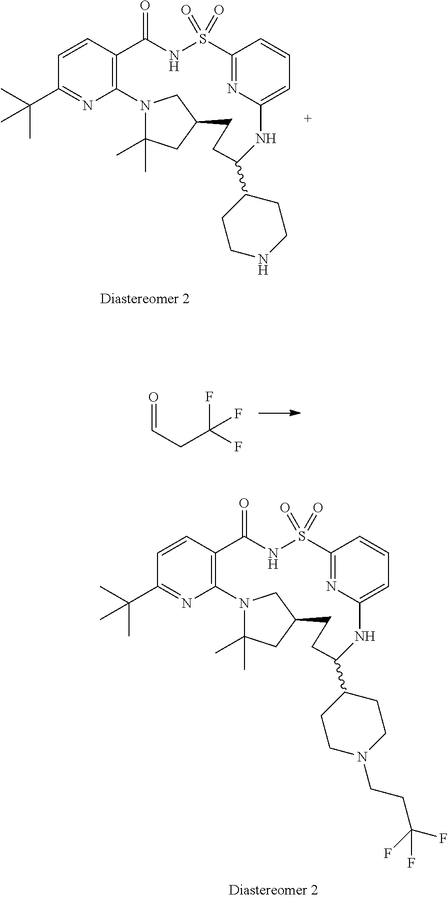 [SMILES: CC(C)(C)c1ccc2c(n1)N1C[C@@H](CCC(C3CCNCC3)Nc3cccc(n3)S(=O)(=O)NC2=O)CC1(C)C ; O=CCC(F)(F)F ; CC(C)(C)c1ccc2c(n1)N1C[C@@H](CCC(C3CCN(CCC(F)(F)F)CC3)Nc3cccc(n3)S(=O)(=O)NC2=O)CC1(C)C]

A solution of (14S)-8-tert-butyl-12,12-dimethyl-17-(piperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (Compound 248 (diastereomer 2), 15 mg, 0.02537 mmol) and 3,3,3-trifluoropropanal (5 mg, 0.04462 mmol) in DCE (500 μL) and was stirred for five minutes, and sodium triacetoxyborohydride (25 mg, 0.1180 mmol) was added. The reaction was stirred for 20 h, and the mixture was quenched with 0.1 mL concentrated HCl, diluted with DMF, filtered, and purified by reverse-phase preparative chromatography utilizing a C₁₈ column and a 10-99% gradient over 15 min of acetonitrile in water (+5 mM HCl) to afford (14S)-8-tert-butyl-12,12-dimethyl-17-[1-(3,3,3-trifluoropropyl)piperidin-4-yl]-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (8.4 mg, 48%) as a white solid. ¹H NMR (500 MHz, DMSO-d₆) δ 12.38 (s, 1H), 10.90 (s, 1H), 7.65-7.54 (m, 2H), 7.13 (d, J=8.8 Hz, 1H), 7.04 (d, J=7.2 Hz, 1H), 6.76 (d, J=8.4 Hz, 1H), 6.64 (d, J=8.0 Hz, 1H), 4.08 (t, J=7.1 Hz, 1H), 3.51 (dd, J=23.3, 11.8 Hz, 2H), 3.38-3.10 (m, 3H), 3.07 (t, J=8.7 Hz, 1H), 2.92 (ddt, J=25.6, 21.2, 10.1 Hz, 4H), 2.62 (t, J=10.4 Hz, 1H), 2.07 (s, 1H), 1.89 (d, J=10.9 Hz, 1H), 1.80 (dd, J=11.8, 5.3 Hz, 1H), 1.74 (s, 1H), 1.66 (d, J=4.3 Hz, 1H), 1.61 (s, 3H), 1.58 (d, J=12.9 Hz, 2H), 1.52 (d, J=12.1 Hz, 1H), 1.47 (s, 3H), 1.45 (s, 1H), 1.44-1.36 (m, 1H), 1.27 (s, 9H), 1.22 (d, J=12.3 Hz, 1H). ESI-MS m/z calc. 650.3226, found 651.2 (M+1)⁺; Retention time: 1.73 minutes (LC method A).

### Example 66: Preparation of (14S)-8-tert-butyl-12,12-dimethyl-17-[1-(pyrimidin-2-yl)piperidin-4-yl]-2λ⁶-thia-3,9,11,18,23-pentaazatetracyclo[17.3.1.111,14.05,10]tetracosa-1(23),5(10),6,8,19,21-hexaene-2,2,4-trione, Compound 218

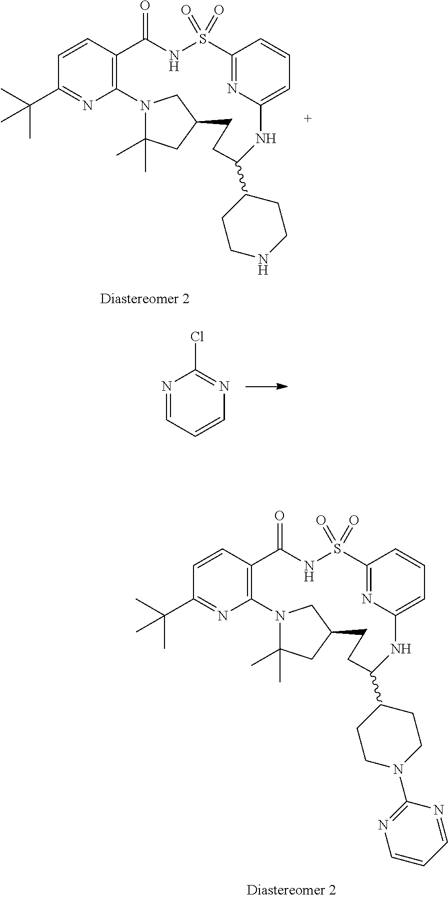 [SMILES: CC(C)(C)c1ccc2c(n1)N1C[C@@H](CCC(C3CCNCC3)Nc3cccc(n3)S(=O)(=O)NC2=O)CC1(C)C ; Clc1ncccn1 ; CC(C)(C)c1ccc2c(n1)N1C[C@@H](CCC(C3CCN(c4ncccn4)CC3)Nc3cccc(n3)S(=O)(=O)NC2=O)CC1(C)C]

To a solution of (14S)-8-tert-butyl-12,12-dimethyl-17-(piperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (Compound 248 (diastereomer 2), 31 mg, 0.05244 mmol) in DMF (1.5 mL) was added K₂CO₃ (21 mg, 0.1519 mmol) followed by 2-chloropyrimidine (6.1 mg, 0.05326 mmol). The reaction was stirred for 20 h, and the mixture was quenched with 0.1 mL concentrated HCl, diluted with DMF, filtered, and purified by reverse-phase preparative chromatography utilizing a C₁₈ column and a 1-99% gradient over 15 min of acetonitrile in water (+5 mM HCl) using a 15 minute run to afford (14S)-8-tert-butyl-12,12-dimethyl-17-[1-(pyrimidin-2-yl)piperidin-4-yl]2λ⁶-thia-3,9,11,18,23-pentaazatetracyclo[17.3.1.111,14.05,10]tetracosa-1(23),5(10),6,8,19,21-hexaene-2,2,4-trione (14.1 mg, 42%) as a white solid. ¹H NMR (500 MHz, DMSO-d₆) δ 12.39 (s, 1H), 8.39 (d, J=4.9 Hz, 2H), 7.61 (d, J=7.9 Hz, 1H), 7.56 (t, J=7.8 Hz, 1H), 7.01 (dd, J=16.0, 7.9 Hz, 2H), 6.73 (d, J=8.5 Hz, 1H), 6.69-6.61 (m, 2H), 4.69 (d, J=13.3 Hz, 1H), 4.63 (d, J=13.0 Hz, 1H), 4.02 (s, 1H), 3.05 (t, J=8.7 Hz, 1H), 2.92 (d, J=13.3 Hz, 1H), 2.86 (t, J=10.8 Hz, 1H), 2.64 (t, J=10.5 Hz, 1H), 2.08 (s, 1H), 1.86 (d, J=13.2 Hz, 1H), 1.80 (dd, J=11.7, 5.3 Hz, 1H), 1.74-1.63 (m, 2H), 1.62 (s, 5H), 1.59-1.49 (m, 2H), 1.48 (s, 3H), 1.27 (s, 11H), 1.17 (d, J=12.0 Hz, 1H). ESI-MS m/z calc. 632.3257, found 633.2 (M+1)⁺; Retention time: 1.91 minutes (LC method A).

The compounds in the following tables were prepared in a manner analogous to that described above, using (14S)-8-tert-butyl-12,12-dimethyl-17-(piperidin-4-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 249 (diastereomer 1) as a starting material:

| | | LCMS | | | |
|---|---|---|---|---|---|
| Compound | | Retention | Exact | | LCMS |
| Number | Structure | Time (min) | Mass | M + 1 | Method |
| Compound 239 (diastereomer 1), hydrochloride salt | 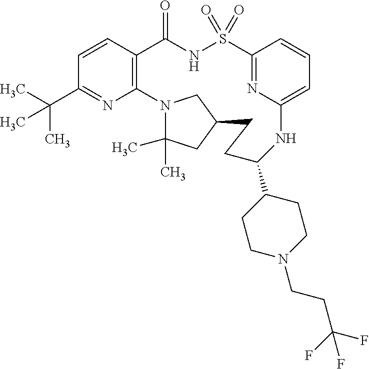 [SMILES: CC(C)(C)c1ccc2c(n1)N1C[C@@H](CC[C@@H](C3CCN(CCC(F)(F)F)CC3)Nc3cccc(n3)S(=O)(=O)NC2=O)CC1(C)C] | 1.61 | 650.323 | 651.3 | LC method A |
| Compound 237 (diastereomer 1), hydrochloride salt | 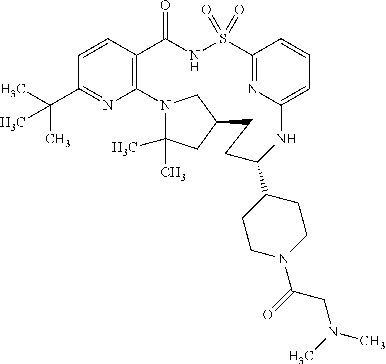 [SMILES: CN(C)CC(=O)N1CCC([C@@H]2CC[C@@H]3CN(c4nc(C(C)(C)C)ccc4C(=O)NS(=O)(=O)c4cccc(n4)N2)C(C)(C)C3)CC1] | 1.54 | 639.357 | 640.2 | LC method A |
| Compound 229 (diastereomer 1) | 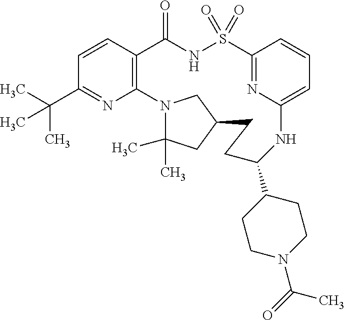 [SMILES: CC(=O)N1CCC([C@@H]2CC[C@@H]3CN(c4nc(C(C)(C)C)ccc4C(=O)NS(=O)(=O)c4cccc(n4)N2)C(C)(C)C3)CC1] | 1.88 | 596.314 | 597.2 | LC method A |
| Compound 219 (diastereomer 1) | 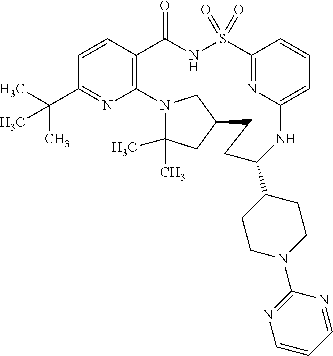 [SMILES: CC(C)(C)c1ccc2c(n1)N1C[C@@H](CC[C@@H](C3CCN(c4ncccn4)CC3)Nc3cccc(n3)S(=O)(=O)NC2=O)CC1(C)C] | 1.84 | 632.326 | 633.4 | LC method A |

| Compound | | |
|---|---|---|
| Number | Structure | NMR |
| Compound 239 (diastereomer 1), hydrochloride salt | 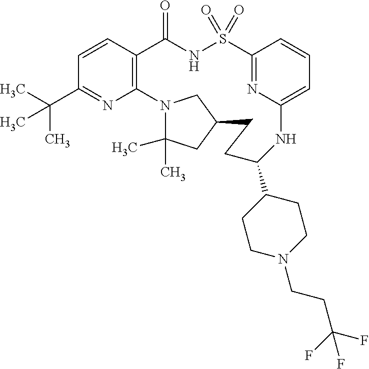 [SMILES: CC(C)(C)c1ccc2c(n1)N1C[C@@H](CC[C@@H](C3CCN(CCC(F)(F)F)CC3)Nc3cccc(n3)S(=O)(=O)NC2=O)CC1(C)C] | ¹H NMR (500 MHz, DMSO-d) δ 12.46 (s, 1H), 11.07 (s, 1H), 7.60 (dt, J = 8.5, 6.1 Hz, 1H), 7.38 (d, J = 41.0 Hz, 2H), 7.15 (dd, J = 46.9, 7.7 Hz, 1H), 6.79 (dd, J = 17.9, 8.5 Hz, 1H), 6.62 (t, J = 8.8 Hz, 1H), 3.53 (d, J = 11.8 Hz, 2H), 3.45- 3.30 (m, 1H), 3.27 (q, J = 8.3, 6.8 Hz, 2H), 3.23-3.05 (m, 1H), 3.00-2.91 (m, 2H), 2.84 (dd, J = 22.0, 11.0 Hz, 2H), 2.24 (s, 1H), 1.85 (td, J= 19.0, 15.8, 9.4 Hz, 4H), 1.73 (s, 1H), 1.63 (d, J = 11.3 Hz, 4H), 1.54 (d, J = 7.1 Hz, 6H), 1.48 (d, J = 16.5 Hz, 1H), 1.42-1.33 (m, 1H), 1.27 (d, J = 5.2 Hz, 9H), 1.18 (dd, J = 27.1, 18.9 Hz, 1H). |
| Compound 229 (diastereomer 1) | 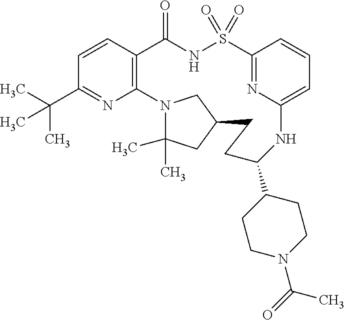 [SMILES: CC(=O)N1CCC([C@@H]2CC[C@@H]3CN(c4nc(C(C)(C)C)ccc4C(=O)NS(=O)(=O)c4cccc(n4)N2)C(C)(C)C3)CC1] | ¹H NMR (500 MHz, DMSO-d₆) δ 12.41 (s, 1H), 7.57 (t, J = 7.8 Hz, 1H), 7.43 (s, 1H), 7.17 (d, J = 7.4 Hz, 2H), 6.77 (d, J = 8.4 Hz, 1H), 6.61 (d, J = 7.9 Hz, 1H), 4.37 (s, 2H), 3.81 (d, J = 13.4 Hz, 1H), 3.13 (d, J = 45.6 Hz, 1H), 2.90 (dt, J = 27.4, 12.7 Hz, 2H), 2.40 (dt, J = 29.0, 12.5 Hz, 1H), 2.24 (d, J = 8.4 Hz, 1H), 1.96 (d, J = 1.9 Hz, 4H), 1.82 (dd, J = 12.0, 5.7 Hz, 1H), 1.67 (dt, J = 20.3, 12.6 Hz, 4H), 1.56 (s, 3H), 1.52 (s, 3H), 1.52-1.44 (m, 1H), 1.28 (s, 1H), 1.28- 1.24 (m, 9H), 1.16 (d, J = 21.5 Hz, 2H), 1.12-0.92 (m, 1H). |
| Compound 219 (diastereomer 1) | 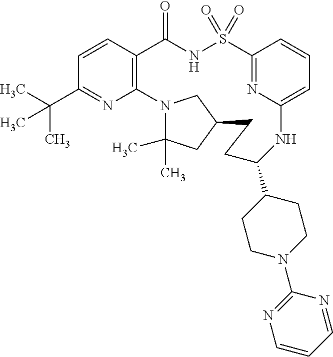 [SMILES: CC(C)(C)c1ccc2c(n1)N1C[C@@H](CC[C@@H](C3CCN(c4ncccn4)CC3)Nc3cccc(n3)S(=O)(=O)NC2=O)CC1(C)C] | ¹H NMR (500 MHz, DMSO-d₆) δ 12.41 (s, 1H), 8.38 (d, J = 4.8 Hz, 2H), 7.57 (t, J = 7.8 Hz, 1H), 7.43 (d, J = 7.9 Hz, 1H), 7.17 (d, J = 7.4 Hz, 2H), 6.77 (d, J = 8.5 Hz, 1H), 6.67-6.59 (m, 2H), 4.67 (d, J = 13.0 Hz, 2H), 3.19-3.01 (m, 1H), 2.83 (dt, J = 26.3, 13.0 Hz, 3H), 2.30-2.21 (m, 1H), 2.08-1.96 (m, 1H), 1.90-1.77 (m, 3H), 1.71 (d, J = 1.7 Hz, 1H), 1.70-1.65 (m, 1H), 1.65-1.59 (m, 1H), 1.55 (d, J = 16.7 Hz, 6H), 1.51- 1.40 (m, 1H), 1.27 (d, J = 1.1 Hz, 9H), 1.26-1.06 (m, 4H). |

### Example 67: Preparation of methyl (14S)-8-tert-Butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylate, Compound 225 (diastereomer 1) and Compound 224 (diastereomer 2); and (14S)-8-tert-Butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylic acid, Compound 231 (diastereomer 1), and Compound 230 (diastereomer 2)

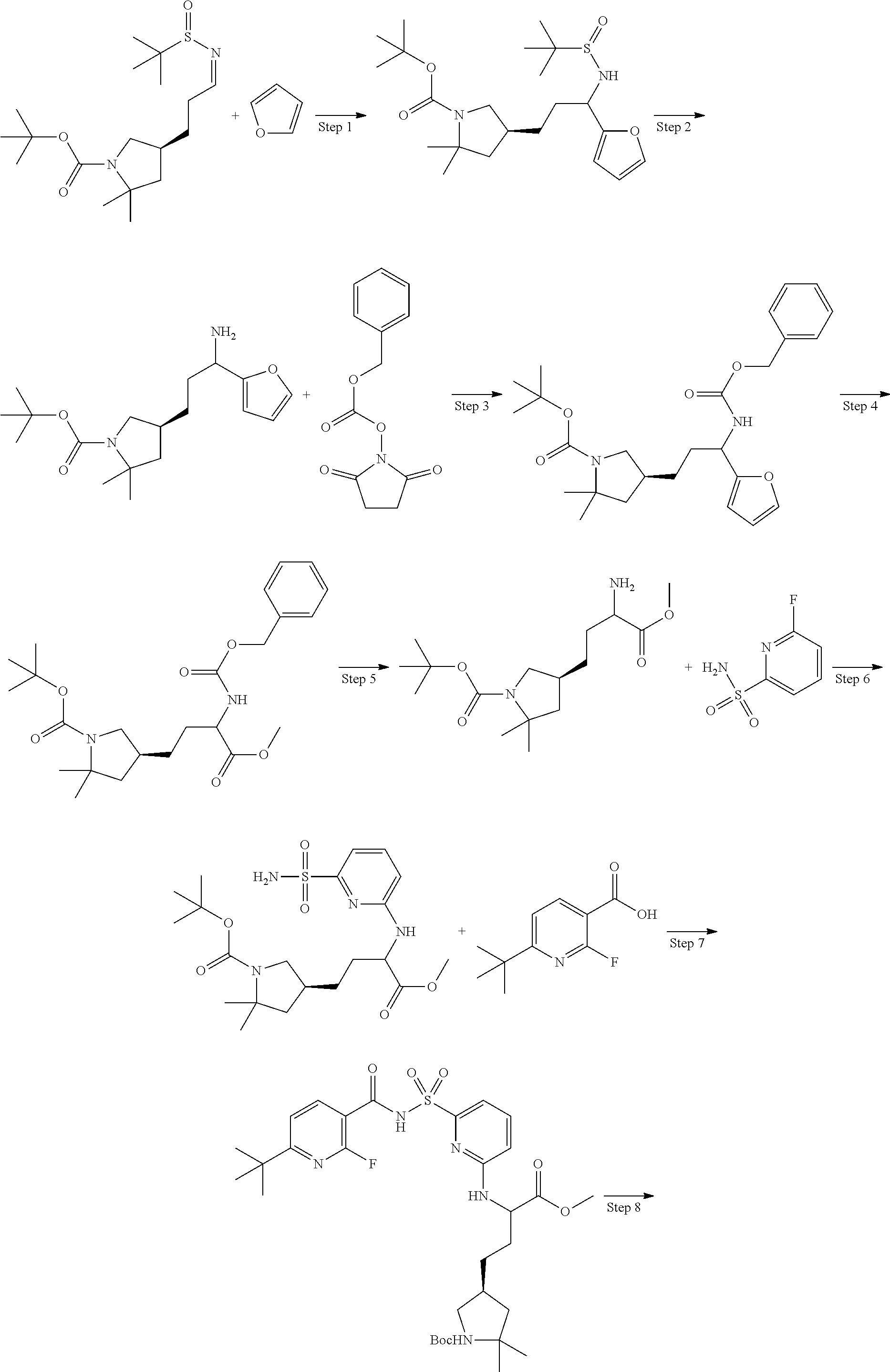

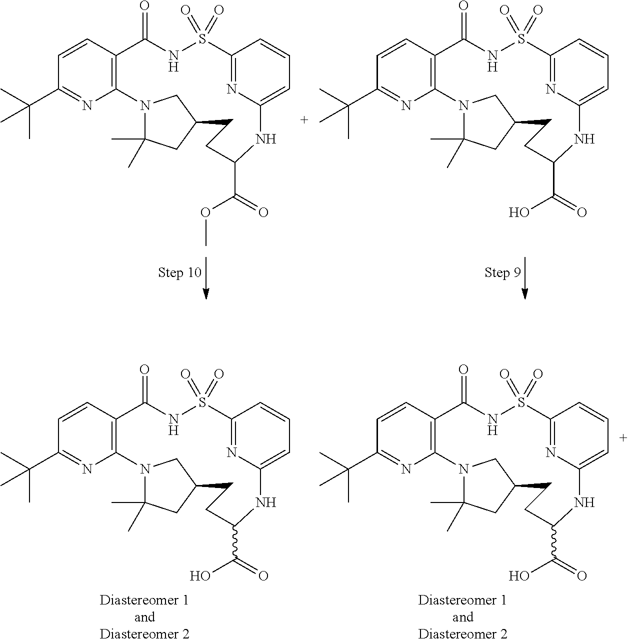

### Step 1: tert-Butyl (4S)-4-[3-(tert-butylsulfinylamino)-3-(2-furyl)propyl]-2,2-dimethyl-pyrrolidine-1-carboxylate

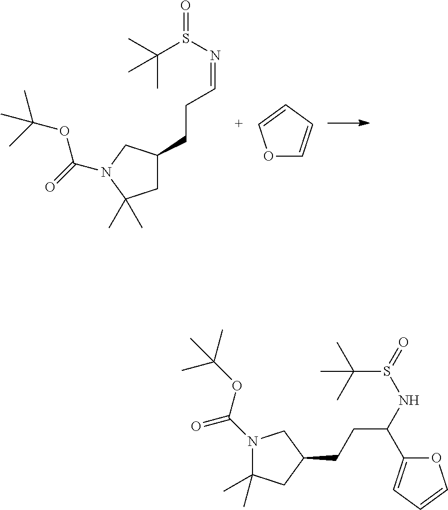 [SMILES: CC(C)(C)OC(=O)N1C[C@@H](CC/C=N\S(=O)C(C)(C)C)CC1(C)C ; c1ccoc1 ; CC(C)(C)OC(=O)N1C[C@@H](CCC(NS(=O)C(C)(C)C)c2ccco2)CC1(C)C]

n-Butyllithium solution (43 mL of 2.5 M in hexanes, 107.50 mmol) was added dropwise over 5 min to a solution of furan (7.8 mL, 107.25 mmol) in THF (350 mL) at −78° C. The mixture was stirred at −78° C. for 15 minutes and room temperature for 2 h. The mixture was re-cooled at −78° C. and tert-butyl (4S)-4-(3-tert-butylsulfinyliminopropyl)-2,2-dimethyl-pyrrolidine-1-carboxylate (34.91 g, 97.367 mmol) in solution in THF (72 mL) was added dropwise over 5 minutes. The reaction was allowed to slowly warming up to room temperature for 2 h. A saturated solution of NH₄Cl (150 mL) and water (150 mL) were added. The product was extracted with EtOAc (3×250 mL), dried over Na₂SO₄, filtrated and evaporated to dryness to afford tert-butyl (4S)-4-[3-(tert-butylsulfinylamino)-3-(2-furyl)propyl]-2,2-dimethyl-pyrrolidine-1-carboxylate (41.77 g, 97%) as an orange oil. ¹H NMR (400 MHz, CDCl₃) δ 7.36 (br. s., 1H), 6.37-6.17 (m, 2H), 4.45-4.30 (m, 1H), 3.80-3.54 (m, 1H), 3.48-3.25 (m, 1H), 2.95-2.73 (m, 1H), 2.08 (d, J=5.1 Hz, 1H), 1.98-1.78 (m, 3H), 1.51-1.15 (m, 26H). ESI-MS m/z calc. 426.2552, found 427.4 (M+1)⁺; Retention time: 1.98 minutes (LC method F).

### Step 2: tert-Butyl (4S)-4-[3-amino-3-(2-furyl)propyl]-2,2-dimethyl-pyrrolidine-1-carboxylate

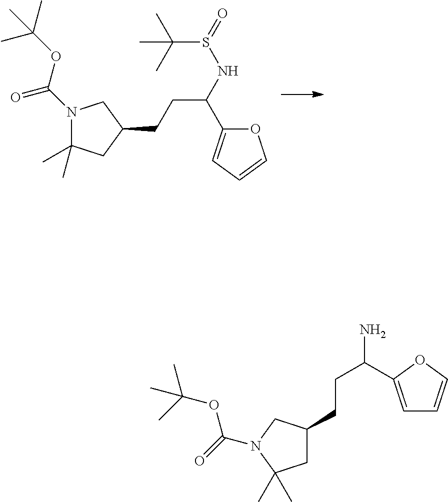 [SMILES: CC(C)(C)OC(=O)N1C[C@@H](CCC(NS(=O)C(C)(C)C)c2ccco2)CC1(C)C ; CC(C)(C)OC(=O)N1C[C@@H](CCC(N)c2ccco2)CC1(C)C]

Molecular iodine (2.44 g, 9.6135 mmol) was added to a solution of tert-butyl (4S)-4-[3-(tert-butylsulfinylamino)-3-(2-furyl)propyl]-2,2-dimethyl-pyrrolidine-1-carboxylate (41.77 g, 94.582 mmol) in THF (700 mL) and water (150 mL). Reaction was stirred at 40° C. for 16 h. Molecular iodine (2.4 g, 0.4868 mL, 9.4559 mmol) was added and the reaction was kept at 40° C. for 24 h. Reaction was diluted with EtOAc (200 mL) and washed with an aqueous solution of sodium thiosulphate 10% (1×300 mL+1×200 mL). The organic layer were combined, washed with brine (300 mL), dried with Na₂SO₄, filtrated and evaporated to dryness. The residue was diluted in EtOAc (300 mL) and washed with a saturated aqueous solution of sodium bicarbonate (2×200 mL), an aqueous solution of sodium thiosulphate 10% and a water and brine solution 1:1 (1×150 mL). The organic layer was dried with Na₂SO₄, filtrated and evaporated to dryness to afford crude tert-butyl(4S)-4-[3-amino-3-(2-furyl)propyl]-2,2-dimethyl-pyrrolidine-1-carboxylate (28.8 g, 83%) as a brown oil which was used in the next step without further purification. ESI-MS m/z calc. 322.2256, found 323.4 (M+1)⁺; Retention time: 1.39 minutes (LC method E).

### Step 3: tert-Butyl (4S)-4-[3-(benzyloxycarbonylamino)-3-(2-furyl)propyl]-2,2-dimethyl-pyrrolidine-1-carboxylate

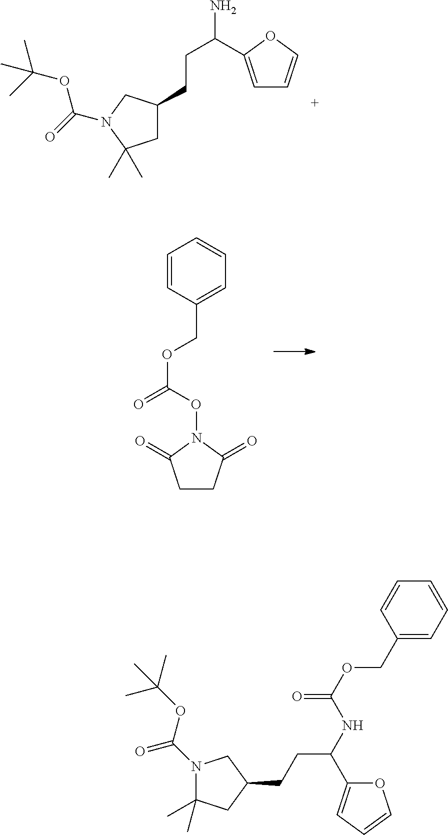 [SMILES: CC(C)(C)OC(=O)N1C[C@@H](CCC(N)c2ccco2)CC1(C)C ; O=C(OCc1ccccc1)ON1C(=O)CCC1=O ; CC(C)(C)OC(=O)N1C[C@@H](CCC(NC(=O)OCc2ccccc2)c2ccco2)CC1(C)C]

Benzyl (2,5-dioxopyrrolidin-1-yl) carbonate (42 g, 168.53 mmol) was added by portion to a solution of tert-butyl (4S)-4-[3-amino-3-(2-furyl)propyl]-2,2-dimethyl-pyrrolidine-1-carboxylate (52.7 g, 161.32 mmol) and triethylamine (17.424 g, 24 mL, 172.19 mmol) in THF (400 mL) under nitrogen and the reaction was stirred at room temperature for 2 h. After completion, imidazole (2.2 g, 32.316 mmol) was added in order to consume the CbzOSu residual and the mixture was stirred for 30 minutes. The reaction was diluted with EtOAc (600 mL) and washed successively with HCl 1N (2×400 mL), 5% potassium carbonate (400 mL) and brine (300 mL). The organic phase was dried over sodium sulfate, filtered and evaporated to dryness to afford tert-butyl (4S)-4-[3-(benzyloxycarbonylamino)-3-(2-furyl)propyl]-2,2-dimethyl-pyrrolidine-1-carboxylate (73.06 g, 97%) as an orange sticky oil. ¹H NMR (400 MHz, CDCl₃) δ 7.41-7.29 (m, 6H), 6.31 (br. s., 1H), 6.18 (br. s., 1H), 5.17-4.98 (m, 3H), 4.87-4.73 (m, 1H), 3.79-3.54 (m, 1H), 2.92-2.76 (m, 1H), 2.16-2.05 (m, 1H), 1.93-1.73 (m, 3H), 1.53-1.28 (m, 18H). ESI-MS m/z calc. 456.2624, found 357.4 (M−99)+; Retention time: 2.075 minutes (LC method E).

### Step 4: tert-Butyl (4S)-4-[3-(benzyloxycarbonylamino)-4-methoxy-4-oxo-butyl]-2,2-dimethyl-pyrrolidine-1-carboxylate

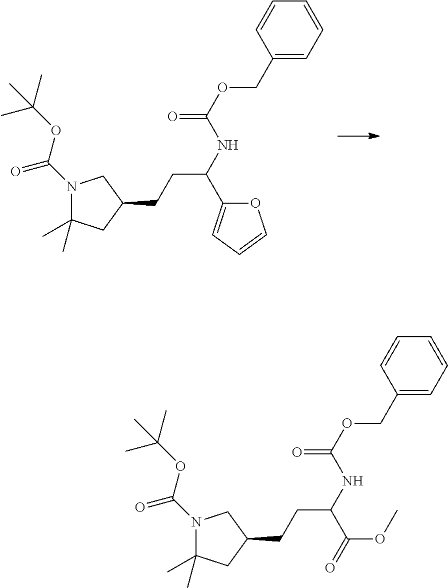 [SMILES: CC(C)(C)OC(=O)N1C[C@@H](CCC(NC(=O)OCc2ccccc2)c2ccco2)CC1(C)C ; COC(=O)C(CC[C@@H]1CN(C(=O)OC(C)(C)C)C(C)(C)C1)NC(=O)OCc1ccccc1]

A solution of sodium periodate (157 g, 734.02 mmol) in water (650 mL) were added to a stirred solution of tert-butyl (4S)-4-[3-(benzyloxycarbonylamino)-3-(2-furyl)propyl]-2,2-dimethyl-pyrrolidine-1-carboxylate (71 g, 122.38 mmol) and ruthenium(III) chloride (1.55 g, 7.4724 mmol) in carbon tetrachloride (275 mL) and acetonitrile (500 mL) at 0° C. The mixture was stirred for 1 hour before it was raised slowly to room temperature and stirred for 2 h. More sodium periodate (65 g, 303.89 mmol) was added and the reaction was continued to stirred for 1 hour while maintaining the temperature below 25° C. (using an ice bath). The mixture was filtered through a pad of celite and washed with ethyl acetate (5×300 mL). A solution of 10% sodium thiosulfate (300 mL) was added to the filtrate and the biphasic mixture was stirred for 10 min and phases were separated. The organic phase was washed with 10% sodium thiosulfate (300 mL) and a 1/1 solution of water/brine (600 mL). The solution was dried over sodium sulfate, filtered and evaporated to dryness. The brown foam, so obtained, was dissolved in dimethylformamide (640 mL). To this stirred solution was added iodomethane (35.340 g, 15.5 mL, 248.98 mmol) followed by cesium carbonate (20 g, 61.384 mmol) and the reaction was stirred at room temperature for 18 h. The reaction was poured onto water (1.2 L) and extracted thrice with ethyl acetate (3×800 mL). The combined organic phases were washed with water/brine (1/1; 2×1.0 L), dried over sodium sulfate, filtered and evaporated to dryness. The residue was purified by dry column vacuum chromatography, eluting from 0% to 20% of EtOAc in heptane. The impure fraction were combined and purified by silica-gel column chromatography on a 330 g column, eluting from 0% to 30% of EtOAc in heptane. All pure fraction were mixed together and evaporated to dryness to yield tert-butyl (4S)-4-[3-(benzyloxycarbonylamino)-4-methoxy-4-oxo-butyl]-2,2-dimethyl-pyrrolidine-1-carboxylate (35 g, 61%) as a pale yellow oil. ¹H NMR (400 MHz, CDCl₃) δ 7.41-7.30 (m, 6H), 5.34-5.23 (m, 1H), 5.12 (s, 2H), 4.44-4.34 (m, 1H), 3.83-3.53 (m, 51-), 2.99-2.74 (m, 1H), 2.16-2.01 (m, 1H), 1.95-1.80 (m, 2H), 1.73-1.62 (m, 1H), 1.50-1.43 (m, 13H), 1.38 (s, 3H). ESI-MS m/z calc. 448.2573, found 349.4 (M−99)+; Retention time: 1.99 minutes (LC method E).

### Step 5: tert-Butyl (4S)-4-(3-amino-4-methoxy-4-oxo-butyl)-2,2-dimethyl-pyrrolidine-1-carboxylate

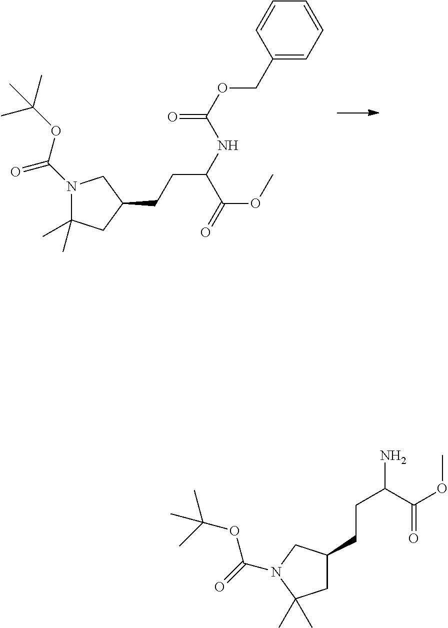 [SMILES: COC(=O)C(CC[C@@H]1CN(C(=O)OC(C)(C)C)C(C)(C)C1)NC(=O)OCc1ccccc1 ; COC(=O)C(N)CC[C@@H]1CN(C(=O)OC(C)(C)C)C(C)(C)C1]

A solution of tert-butyl (4S)-4-[3-(benzyloxycarbonylamino)-4-methoxy-4-oxo-butyl]-2,2-dimethyl-pyrrolidine-1-carboxylate (36.2 g, 76.669 mmol) in MeOH (620 mL) was degassed with nitrogen for 10 minutes. Palladium on carbon (8.4 g, 10% w/w, 7.8933 mmol) was added and hydrogen was bubbled for 1 hour. The reaction was stirred under hydrogen atmosphere for 16 h. The reaction was filtered through a pad of celite and washed with methanol (2×100 mL). After evaporation of the volatile, the crude was dissolved in ethyl acetate (400 mL) and extracted twice with HCl 1N (2×250 mL). The aqueous phase was basified using NaOH 1.5N (450 mL) until pH reached about 9-10 and the trouble solution was extracted twice with ethyl acetate (2×400 mL). The organic phase was washed with a solution 1/1 of water/brine (500 mL), dried over sodium sulfate, filtered and evaporated to dryness to yield tert-butyl (4S)-4-(3-amino-4-methoxy-4-oxo-butyl)-2,2-dimethyl-pyrrolidine-1-carboxylate (18.68 g, 76%) as a yellowish oil. ¹H NMR (400 MHz, CDCl₃) δ 3.78-3.58 (m, 4H), 3.49-3.39 (m, 1H), 2.96-2.81 (m, 1H), 2.17-2.06 (m, 1H), 1.97-1.83 (m, 1H), 1.81-1.67 (m, 1H), 1.65-1.53 (m, 1H), 1.53-1.36 (m, 18H). ESI-MS m/z calc. 314.2206, found 315.4 (M+1)⁺; Retention time: 1.325 minutes (LC method E).

### Step 6: tert-Butyl (4S)-4-[4-methoxy-4-oxo-3-[(6-sulfamoyl-2-pyridyl)amino]butyl]-2,2-dimethyl-pyrrolidine-1-carboxylate

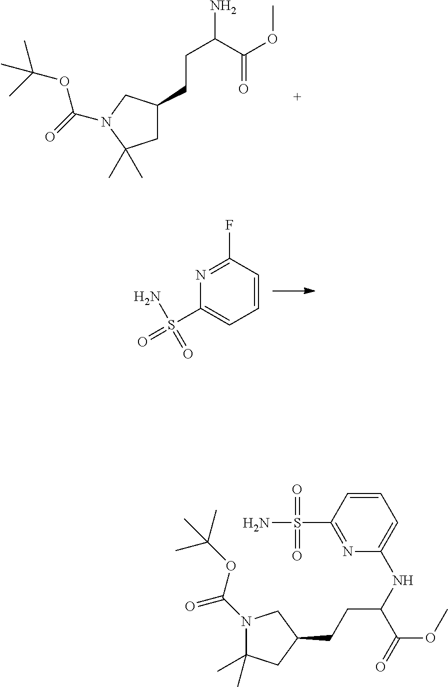

A solution of tert-butyl (4S)-4-(3-amino-4-methoxy-4-oxo-butyl)-2,2-dimethyl-pyrrolidine-1-carboxylate (18 g, 56.218 mmol), 6-fluoropyridine-2-sulfonamide (22.7 g, 128.85 mmol) and diisopropylethylamine (20.034 g, 27 mL, 155.01 mmol) in dimethylsulfoxide (90 mL) and dioxane (20 mL) was stirred at 120° C. for 40 h. After cooling down to room temperature, the reaction was diluted with ethyl acetate (2.0 L) and washed twice with a solution 1/1 of water/brine (2×1.0 L). The organic phase was dried over sodium sulfate, filtered and evaporated to dryness. The residue was purified by dry column vacuum chromatography, eluting from 10% to 60% of EtOAc in heptane, and by reverse phase chromatography (in 4 batches) on a 275 g C₁₈ column, eluting from 10% to 100% MeOH in water to yield tert-butyl (4S)-4-[4-methoxy-4-oxo-3-[(6-sulfamoyl-2-pyridyl)amino]butyl]-2,2-dimethyl-pyrrolidine-1-carboxylate (17.88 g, 66%) as a white solid. ¹H NMR (400 MHz, DMSO-d₆) δ 7.58 (t, J=7.8 Hz, 1H), 7.39 (dd, J=7.8, 3.2 Hz, 1H), 7.07-6.98 (m, 3H), 6.76 (d, J=8.3 Hz, 1H), 4.65 (br. s., 1H), 3.63 (s, 3H), 3.59-3.47 (m, 1H), 2.85-2.72 (m, 1H), 2.18-2.01 (m, 1H), 1.94-1.61 (m, 3H), 1.46-1.30 (m, 15H), 1.23 (s, 3H). ESI-MS m/z calc. 470.2199, found 415.1 (M−55)+; Retention time: 4.07 minutes (LC method F).

### Step 7: tert-Butyl (4S)-4-[3-[[6-[(6-tert-butyl-2-fluoro-pyridine-3-carbonyl)sulfamoyl]-2-pyridyl]amino]-4-methoxy-4-oxo-butyl]-2,2-dimethyl-pyrrolidine-1-carboxylate

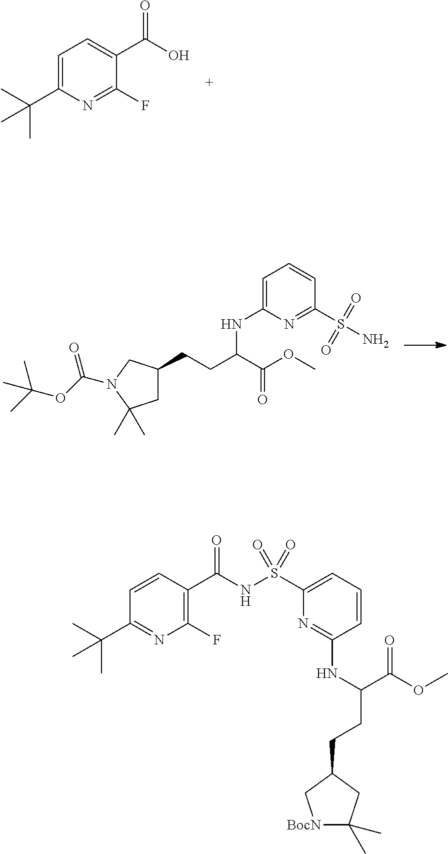 [SMILES: CC(C)(C)c1ccc(C(=O)O)c(F)n1 ; COC(=O)C(CC[C@@H]1CN(C(=O)OC(C)(C)C)C(C)(C)C1)Nc1cccc(S(N)(=O)=O)n1 ; COC(=O)C(CC[C@@H]1CN(C(=O)OC(C)(C)C)C(C)(C)C1)Nc1cccc(S(=O)(=O)NC(=O)c2ccc(C(C)(C)C)nc2F)n1]

In a 250-mL round-bottomed flask, 6-tert-butyl-2-fluoro-pyridine-3-carboxylic acid (6.03 g, 30.58 mmol) was dissolved in THF (100 mL), to which CDI (5.05 g, 31.14 mmol) was added. The resulting mixture was stirred at room temperature for 22 h. After this time, tert-butyl (4S)-4-[4-methoxy-4-oxo-3-[(6-sulfamoyl-2-pyridyl)amino]butyl]-2,2-dimethyl-pyrrolidine-1-carboxylate (10.02 g, 20.74 mmol) and DBU (12 mL, 80.24 mmol) were added, and the resulting mixture was stirred at room temperature for 22 h. After this time, the mixture was poured into EtOAc (300 mL). This mixture was then washed with saturated aqueous sodium bicarbonate solution (150 mL), aqueous HCl solution (0.1 N, 150 mL) and saturated aqueous NaCl solution (150 mL), then dried over Na₂SO₄, filtered, and evaporated in vacuo. The resulting brown foam was purified by silica gel chromatography (330 g of silica) using a gradient eluent of 0 to 100% EtOAc in hexanes to give a white foam, tert-butyl (4S)-4-[3-[[6-[6-tert-butyl-2-fluoro-pyridine-3-carbonyl)sulfamoyl]-2-pyridyl]amino]-4-methoxy-4-oxo-butyl]-2,2-dimethyl-pyrrolidine-1-carboxylate (4.439 g, 33%); ESI-MS m/z calc. 649.29456, found 650.3 (M+1)⁺; Retention time: 2.11 minutes (LC method A).

### Step 8: Methyl (14S)-8-tert-Butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylate and (14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylic acid (diastereomeric mixtures)

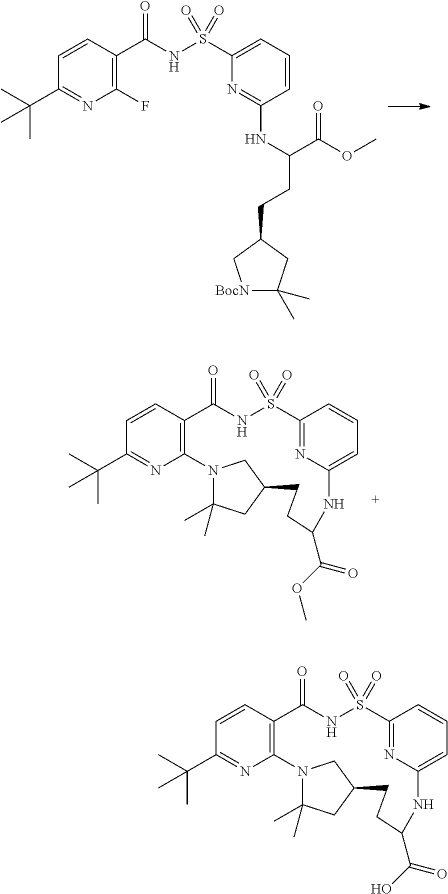

Stage 1: In a 250-mL round-bottomed flask, tert-butyl (4S)-4-[3-[[6-[(6-tert-butyl-2-fluoro-pyridine-3-carbonyl)sulfamoyl]-2-pyridyl]amino]-4-methoxy-4-oxo-butyl]-2,2-dimethyl-pyrrolidine-1-carboxylate (4.5 g, 6.925 mmol), was dissolved in dichloromethane (100 mL). TFA (12.0 mL, 155.8 mmol) was added, and the resulting solution was allowed to stand at room temperature for 4 h. The mixture was then evaporated in vacuo, diluted with dioxane, and evaporated in vacuo again. This gave an orange foam, ˜5 g (>100% yield).

Stage 2: In a 250-mL round-bottomed flask, the crude product from Step 1 was dissolved in NMP (100 mL), to which K₂CO₃ (13.0 g, 94.06 mmol) was added. The resulting mixture was flushed with nitrogen, then stirred at 150° C. for 20 h. After cooling to room temperature, the reaction mixture was poured into aqueous HCl solution (1 N; 200 mL), then extracted with EtOAc (2×300 mL). The combined organic extracts was washed with H₂O (300 mL) and saturated aqueous NaCl solution (300 mL), then dried over Na₂SO₄, filtered, and evaporated in vacuo. A brown foam was obtained as the crude product. This was purified by a silica gel plug (150 g of silica; 500 mL of EtOAc) to give two batches of product. This corresponded to ester (˜1.0 g) and acid (˜1.5 g) products, since the ester had partially hydrolyzed under the reaction conditions. These two products were further purified in the subsequent step.

Methyl (14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylate (1.0 g, 27%) ESI-MS m/z calc. 529.2359, found 530.2 (M+1)⁺; Retention time: 0.74 minutes (LC method D).

(14S)-8-tert-Butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylic acid (1.5 g, 42%) ESI-MS m/z calc. 515.2202, found 516.2 (M+1)⁺; Retention time: 0.69 minutes and 0.71 minutes (LC method D).

### Step 9: (14S)-8-tert-Butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylic acid, diastereomer 1, Compound 231, and (14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylic acid, diastereomer 2, Compound 230

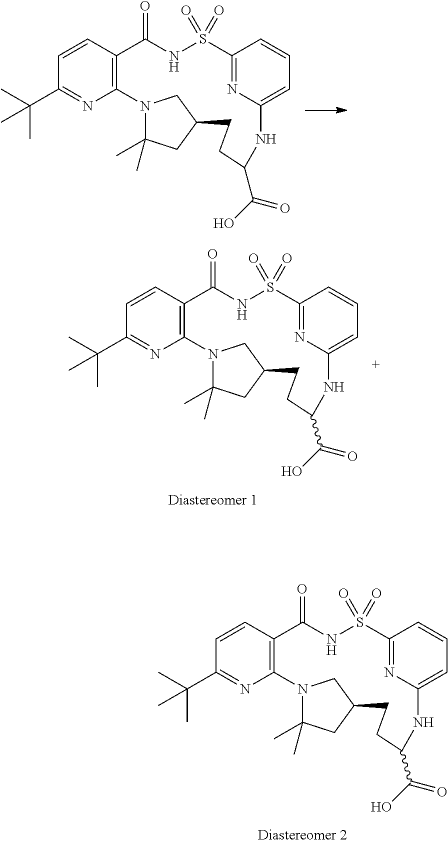

The semi-purified product from step 8 containing (14S)-8-tert-butyl-12,12-dimethyl-2,2,4-tri oxo-2λ⁶-thia-3,9,11,18,23-pentaazatetracyclo[17.3.1.111,14.05,10]tetracosa-1(22),5,7,9,19(23),20-hexaene-17-carboxylic acid (1.5 g, 2.909 mmol)] was purified by silica gel chromatography (330 g of silica) using a gradient eluent of 0 to 100% EtOAc in hexanes, followed by many batches of reverse-phase chromatography, to give:

Diastereomer 1, shorter retention time on UPLC: (14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylic acid (203.9 mg, 14%); ¹H NMR (400 MHz, DMSO-d₆) δ 12.57 (s, 1H), 12.43 (s, 1H), 7.63 (dd, J=8.5, 7.3 Hz, 1H), 7.51 (d, J=7.8 Hz, 1H), 7.41 (d, J=7.9 Hz, 1H), 7.25 (d, J=7.2 Hz, 1H), 6.87 (d, J=8.4 Hz, 1H), 6.61 (d, J=7.9 Hz, 1H), 4.37-4.07 (m, 1H), 3.16-3.01 (m, 1H), 2.97-2.77 (m, 1H), 2.36-2.25 (m, 1H), 2.25-2.06 (m, 1H), 2.04-1.92 (m, 1H), 1.84 (dd, J=11.8, 5.9 Hz, 1H), 1.75-1.65 (m, 1H), 1.57 (s, 3H), 1.56-1.46 (m, 1H), 1.52 (s, 3H), 1.26 (s, 9H), 1.25-1.16 (m, 1H); ESI-MS m/z calc. 515.2202, found 516.2 (M+1)⁺; Retention time: 1.75 minutes (LC method A).

Diastereomer 2, longer retention time on UPLC: (14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylic acid (369.2 mg, 25%); ¹H NMR (400 MHz, DMSO-d₆) δ 12.53 (s, 1H), 7.64 (dd, J=8.5, 7.2 Hz, 1H), 7.60 (d, J=8.0 Hz, 1H), 7.45 (d, J=9.4 Hz, 1H), 7.12 (d, J=7.1 Hz, 1H), 6.83 (d, J=8.4 Hz, 1H), 6.65 (d, J=8.0 Hz, 1H), 4.73-4.62 (m, 1H), 3.06 (dd, J=10.2, 6.8 Hz, 1H), 2.66 (t, J=10.4 Hz, 1H), 2.24-2.10 (m, 1H), 1.92 (t, J=12.9 Hz, 1H), 1.85-1.78 (m, 1H), 1.69 (dd, J=13.9, 5.5 Hz, 1H), 1.63 (s, 3H), 1.53 (t, J=12.2 Hz, 1H), 1.49 (s, 3H), 1.40-1.28 (m, 1H), 1.27 (s, 9H). [Note: 1H is missing—either the sulfonamide or the carboxylic acid 1H is deprotonated, or is blended in with the water peak.] ESI-MS m/z calc. 515.2202, found 516.1 (M+1)⁺; Retention time: 1.82 minutes (LC method A).

### Step 10: Methyl (14S)-8-tert-Butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylate, diastereomer 1, Compound 225, and methyl (14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylate, diastereomer 2, Compound 224

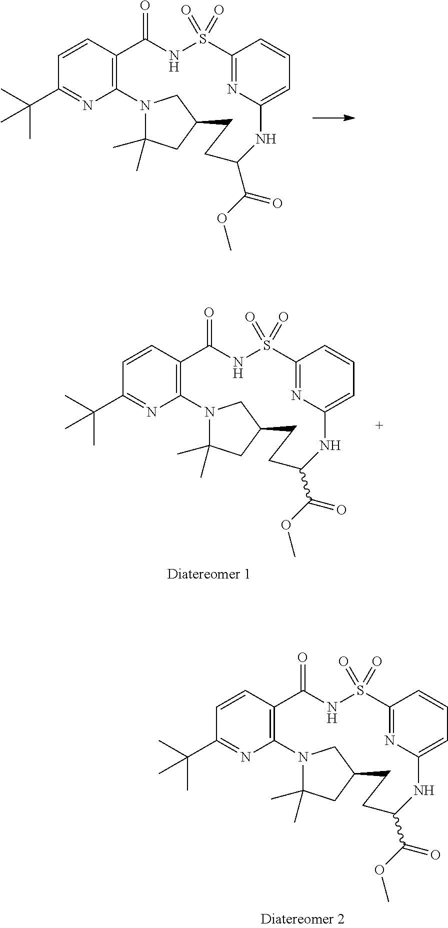 [SMILES: COC(=O)C1CC[C@@H]2CN(c3nc(C(C)(C)C)ccc3C(=O)NS(=O)(=O)c3cccc(n3)N1)C(C)(C)C2 ; COC(=O)C1CC[C@@H]2CN(c3nc(C(C)(C)C)ccc3C(=O)NS(=O)(=O)c3cccc(n3)N1)C(C)(C)C2 ; COC(=O)C1CC[C@@H]2CN(c3nc(C(C)(C)C)ccc3C(=O)NS(=O)(=O)c3cccc(n3)N1)C(C)(C)C2]

The semi-purified product from step 8 [containing methyl (14S)-8-tert-butyl-12,12-dimethyl-2,2,4-tri oxo-2λ⁶-thia-3,9,11,18,23-pentaazatetracyclo[17.3.1.111,14.05,10]tetracosa-1(22),5,7,9,19(23),20-hexaene-17-carboxylate (1.0 g, 1.888 mmol)] was purified by silica gel chromatography (330 g of silica) using a gradient eluent of 0 to 100% EtOAc in hexanes to give 2 products:

Diastereomer 1, eluted earlier from SiO2 column: Methyl (14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylate (170.3 mg, 17%); ¹H NMR (400 MHz, DMSO-d₆) δ 12.47 (s, 1H), 7.72-7.54 (m, 2H), 7.40 (d, J 7.7 Hz, 1H), 7.27 (d, J=7.3 Hz, 1H), 6.86 (d, J=8.4 Hz, 1H), 6.61 (d, J=7.9 Hz, 1H), 4.46-4.17 (m, 1H), 3.61 (s, 3H), 3.17-3.03 (m, 1H), 2.86-2.69 (m, 1H), 2.38-2.14 (m, 2H), 1.98-1.92 (m, 1H), 1.84 (dd, J=11.8, 5.8 Hz, 1H), 1.75-1.64 (m, 1H), 1.56 (s, 3H), 1.52 (s, 3H), 1.51-1.44 (m, 1H), 1.37-1.27 (m, 1H), 1.26 (s, 9H); ESI-MS m/z calc. 529.2359, found 530.2 (M+1)⁺; Retention time: 1.96 minutes (LC method A).

Diastereomer 2, eluted later from SiO2 column: Methyl (14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylate (538.0 mg, 54%); ¹H NMR (400 MHz, DMSO-d₆) δ 12.56 (s, 1H), 7.67 (dd, J=8.4, 7.2 Hz, 1H), 7.60 (d, J=8.0 Hz, 1H), 7.56 (d, J=9.5 Hz, 1H), 7.15 (d, J=7.2 Hz, 1H), 6.84 (d, J 8.3 Hz, 1H), 6.65 (d, J=8.0 Hz, 1H), 4.78 (ddd, J=12.8, 9.5, 3.1 Hz, 1H), 3.63 (s, 3H), 3.02 (dd, J=10.2, 6.8 Hz, 1H), 2.65 (t, J=10.4 Hz, 1H), 2.24-2.09 (m, 1H), 1.94-1.77 (m, 3H), 1.70 (dd, J=14.3, 5.5 Hz, 1H), 1.63 (s, 3H), 1.53 (t, J=12.7 Hz, 1H), 1.48 (s, 3H), 1.40-1.28 (m, 1H), 1.27 (s, 9H); ESI-MS m/z calc. 529.2359, found 530.2 (M+1)⁺; Retention time: 1.96 minutes (LC method A).

### Example 68: Preparation of (14S)-8-tert-Butyl-17-(2-hydroxypropan-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 12

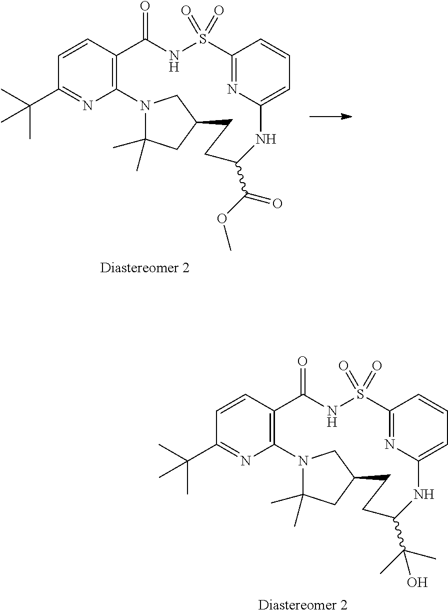 [SMILES: COC(=O)C1CC[C@@H]2CN(c3nc(C(C)(C)C)ccc3C(=O)NS(=O)(=O)c3cccc(n3)N1)C(C)(C)C2 ; CC(C)(C)c1ccc2c(n1)N1C[C@@H](CCC(C(C)(C)O)Nc3cccc(n3)S(=O)(=O)NC2=O)CC1(C)C]

In a 3-mL vial, methyl (14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylate (Compound 224 (diastereomer 2), 41.4 mg, 0.07816 mmol) was dissolved in THF (400 μL) and cooled to 0° C. Then, a diethyl ether solution of MeMgBr (200 μL of 3.0 M, 0.6000 mmol) was added in one portion. The resulting mixture was stirred at room temperature for 17 h, after which it was quenched with saturated aqueous NH₄Cl solution (1 mL). EtOAc (1.2 mL) was added, then the phases were vigorously mixed and allowed to settle into two layers. The organic layer was filtered and purified by reverse-phase preparative chromatography using a C₁₈ column and a gradient eluent of 1 to 99% acetonitrile in water containing a 5 mM HCl solution to give (14S)-8-tert-butyl-17-(2-hydroxypropan-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (14.2 mg, 34%); ¹H NMR (400 MHz, DMSO-d₆) δ 12.40 (s, 1H), 7.60 (d, J=7.9 Hz, 1H), 7.55 (dd, J=8.5, 7.2 Hz, 1H), 7.01 (dd, J=7.2, 0.7 Hz, 1H), 6.89-6.80 (m, 1H), 6.77 (dd, J=8.5, 0.9 Hz, 1H), 6.64 (d, J=8.0 Hz, 1H), 3.89 (t, J=10.6 Hz, 1H), 3.09 (dd, J=10.4, 6.7 Hz, 1H), 2.62 (t, J=10.5 Hz, 1H), 2.07-1.94 (m, 1H), 1.80 (dd, J=11.6, 5.3 Hz, 1H), 1.73 (t, J=12.7 Hz, 1H), 1.68-1.55 (m, 2H), 1.61 (s, 3H), 1.55-1.48 (m, 1H), 1.48 (s, 3H), 1.27 (s, 9H), 1.23-1.13 (m, 1H), 1.10 (s, 3H), 1.03 (s, 3H). [Note: The OH signal is hidden under the residual water peak.]. ESI-MS m/z calc. 529.2723, found 530.2 (M+1)⁺; Retention time: 1.93 minutes (LC method A).

### Example 69: Preparation of (14S)—N-{7-Azaspirop[3.5]nonan-2-yl}-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaene-17-carboxamide, Compound 222 (diastereomer 2)

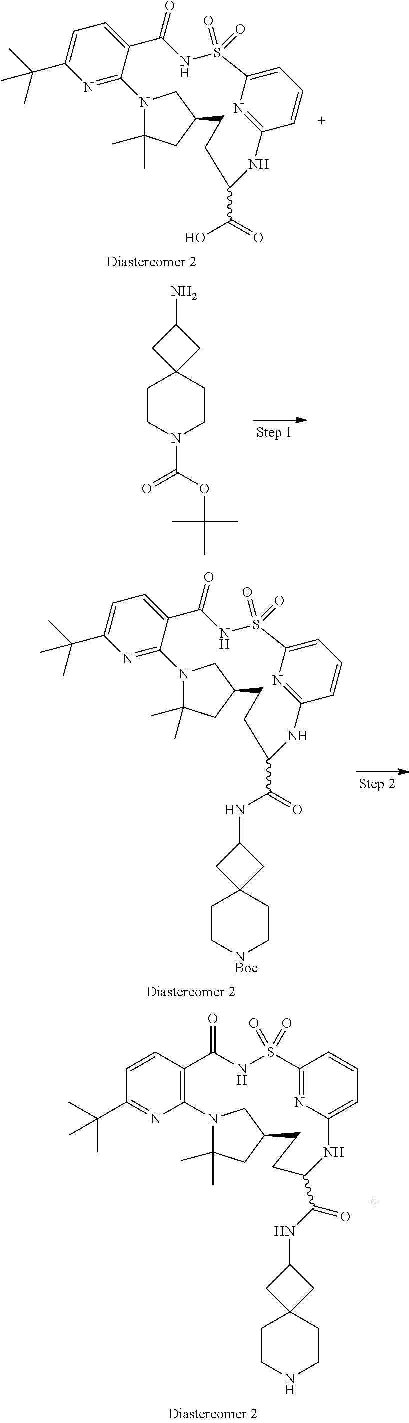

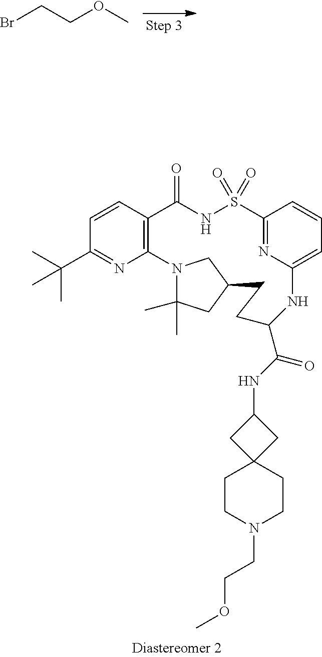

### Step 1: tert-Butyl 2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-amido]-7-azaspiro[3.5]nonane-7-carboxylate, Compound 226 (diastereomer 2)

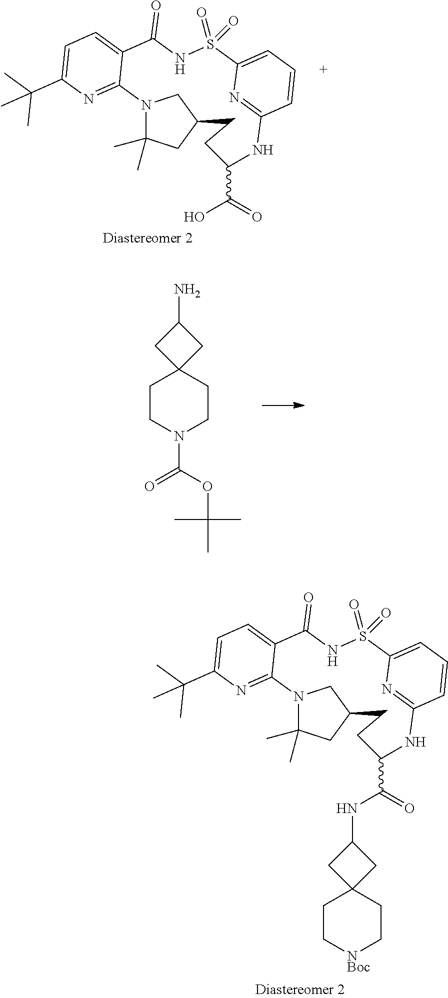 [SMILES: CC(C)(C)c1ccc2c(n1)N1C[C@@H](CCC(C(=O)O)Nc3cccc(n3)S(=O)(=O)NC2=O)CC1(C)C ; CC(C)(C)OC(=O)N1CCC2(CC1)CC(N)C2 ; CC(C)(C)OC(=O)N1CCC2(CC1)CC(NC(=O)C1CC[C@@H]3CN(c4nc(C(C)(C)C)ccc4C(=O)NS(=O)(=O)c4cccc(n4)N1)C(C)(C)C3)C2]

In a 100-mL flask, (14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylic acid (diastereomer 2, Compound 230, 40.3 mg, 0.07761 mmol) was dissolved in DMF (2 mL), to which HATU (36.89 mg, 0.09702 mmol) was added and then tert-butyl 2-amino-7-azaspiro[3.5]nonane-7-carboxylate (20.5 mg, 0.08538 mmol) was added followed by DIPEA (35, 0.2716 mmol). After stirring at room temperature for 16 h, diluted with EtOAc and washed with 1N HCl (1×), saturated brine (1×), dried (sodium sulfate), filtered and concentrated to an off-white solid which was filtered and purified using a reverse phase HPLC-MS method using a Luna C₁₈ column (75×30 mm, 5 μm particle size) sold by Phenomenex (pn: 00C-4252-U0-AX), and a dual gradient run from 30-99% mobile phase B over 15.0 minutes (mobile phase A=H₂O (5 mM HCl), mobile phase B=acetonitrile, flow rate=50 mL/min, injection volume=950 μL and column temperature=25° C.) giving tert-butyl 2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-amido]-7-azaspiro[3.5]nonane-7-carboxylate (41.5 mg, 72%) ESI-MS m/z calc. 737.39343, found 738.8 (M+1)⁺; Retention time: 0.85 minutes (LC method D). ESI-MS m/z calc. 737.39343, found 738.2 (M+1)⁺; Retention time: 2.19 minutes (LC method A).

### Step 2: (14S)—N-{7-Azaspiro[3.5]nonan-2-yl}-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaene-17-carboxamide, diastereomer 2, Compound 222

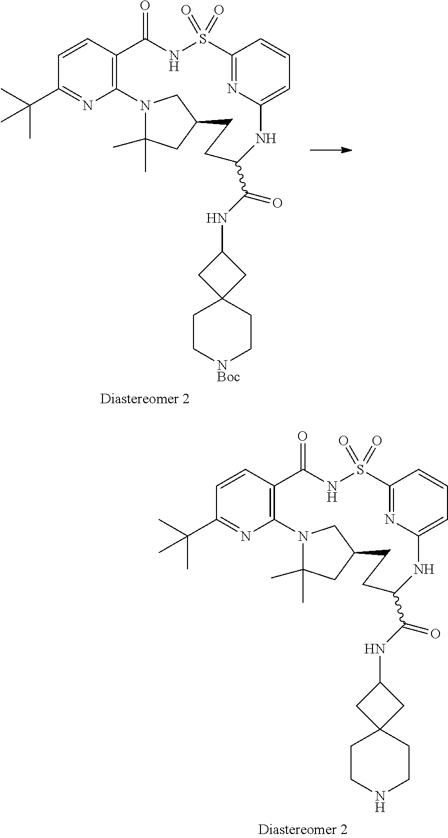 [SMILES: CC(C)(C)OC(=O)N1CCC2(CC1)CC(NC(=O)C1CC[C@@H]3CN(c4nc(C(C)(C)C)ccc4C(=O)NS(=O)(=O)c4cccc(n4)N1)C(C)(C)C3)C2 ; CC(C)(C)c1ccc2c(n1)N1C[C@@H](CCC(C(=O)NC3CC4(CCNCC4)C3)Nc3cccc(n3)S(=O)(=O)NC2=O)CC1(C)C]

tert-Butyl 2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-amido]-7-azaspiro[3.5]nonane-7-carboxylate (Compound 226 (diastereomer 2), 38 mg, 0.05098 mmol) was dissolved in DCM (10004) and to the mixture was added TFA (200 μL, 2.596 mmol) and stirred at room temperature for 1 h. The mixture was evaporated to dryness, then diluted with diethyl ether (30 mL×2), and reconcentrated. This mixture was diluted with DMSO and was then purified by reverse-phase preparative chromatography utilizing a C₁₈ column and a 1-70% gradient over 15 min of acetonitrile in water (+5 mM HCl) to afford as a white solid (14S)—N-{7-azaspiro[3.5]nonan-2-yl}-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaene-17-carboxamide (hydrochloride salt) as a white solid (diastereomer 2, 33.2 mg, 96%). ESI-MS m/z calc. 637.341, found 638.2 (M+1)⁺; Retention time: 1.59 minutes (LC method A).

### Step 3: (14S)-8-tert-Butyl-N-[7-(2-methoxyethyl)-7-azaspiro[3.5]nonan-2-yl]-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaene-17-carboxamide, Compound 220 (diastereomer 2)

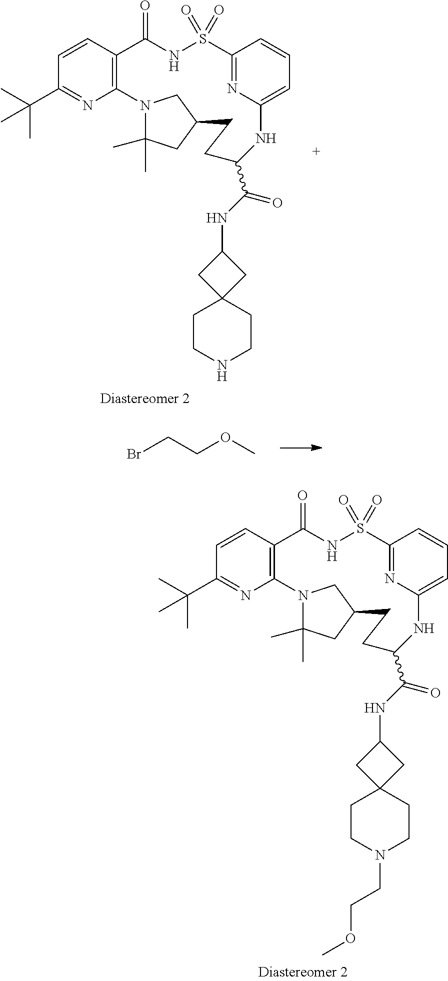

To a solution of (14S)—N-{7-azaspiro[3.5]nonan-2-yl}-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaene-17-carboxamide (hydrochloride salt) (30 mg, 0.04405 mmol) in DMF (1.5 mL) was added K₂CO₃ (30 mg, 0.2171 mmol) followed by 1-bromo-2-methoxy-ethane (4.2 μL, 0.04469 mmol) in 424 DMF. The reaction was stirred at 50° C. for 22 h. Then the mixture was quenched with 0.1 mL concentrated HCl, diluted with DMF, filtered, and purified by reverse-phase preparative chromatography utilizing a C₁₈ column and a 20-65% gradient over 30 min of acetonitrile in water (+5 mM HCl) to afford as a white solid (14S)-8-tert-butyl-N-[7-(2-methoxyethyl)-7-azaspiro[3.5]nonan-2-yl]-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaene-17-carboxamide (hydrochloride salt) (diastereomer 2, 5.7 mg, 17%). ¹H NMR (500 MHz, DMSO-d₆) δ 12.56 (d, J=7.9 Hz, 1H), 10.37-10.09 (m, 1H), 8.14 (d, J=5.2 Hz, 1H), 7.72-7.54 (m, 2H), 7.46-7.30 (m, 1H), 7.13 (d, J=7.2 Hz, 1H), 6.87 (d, J=8.4 Hz, 1H), 6.63 (d, J 7.9 Hz, 1H), 4.48 (t, J=9.9 Hz, 1H), 4.16 (dd, J=16.7, 8.9 Hz, 2H), 3.69 (t, J=5.0 Hz, 2H), 3.32 (d, J 13.3 Hz, 1H), 3.27 (s, 3H), 3.19 (d, J 5.2 Hz, 2H), 3.16-3.08 (m, 1H), 2.93-2.85 (m, 1H), 2.82-2.72 (m, 1H), 2.60-2.54 (m, 1H), 2.26-2.19 (m, 1H), 2.19-2.11 (m, 1H), 2.06-1.97 (m, 1H), 1.87-1.75 (m, 6H), 1.74-1.70 (m, 1H), 1.69 (d, J 2.8 Hz, 1H), 1.64 (s, 3H), 1.62 (d, J=3.3 Hz, 1H), 1.51 (d, J=12.2 Hz, 1H), 1.47 (s, 3H), 1.27 (s, 9H), 1.26-1.19 (m, 1H). ESI-MS m/z calc. 695.3829, found 696.3 (M+1)⁺; Retention time: 1.63 minutes (LC method A).

The compounds in the following tables were prepared in a manner analogous to that described above, using (14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylic acid, diastereomer 1, Compound 231, or (14S)-8-tert-butyl-12,12-dimethyl-2,2,4-trioxo-2λ⁶-thia-3,9,11,18,23-pentaazatetracyclo[17.3.1.111,14.05,10]tetracosa-1(22),5,7,9,19(23),20-hexaene-17-carboxylate, diastereomer 1, Compound 225 as starting materials:

| | | LCMS | | | |
|---|---|---|---|---|---|
| Compound | | Retention | Exact | | LCMS |
| Number | Structure | Time (min) | Mass | M + 1 | Method |
| Compound 13 (diasteromer 1) | 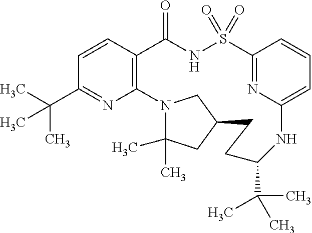 [SMILES: CC(C)(C)c1ccc2c(n1)N1C[C@@H](CC[C@@H](C(C)(C)C)Nc3cccc(n3)S(=O)(=O)NC2=O)CC1(C)C] | 1.85 | 529.272 | 530.2 | LC method A |
| Compound 221 (diasteromer 1), hydrochloride salt | 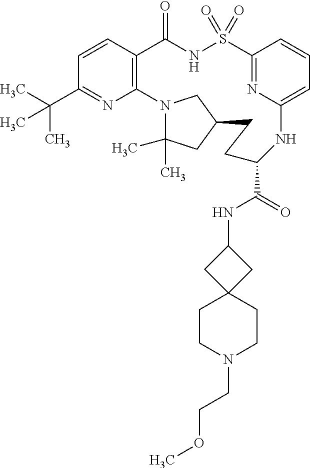 [SMILES: COCCN1CCC2(CC1)CC(NC(=O)[C@@H]1CC[C@@H]3CN(c4nc(C(C)(C)C)ccc4C(=O)NS(=O)(=O)c4cccc(n4)N1)C(C)(C)C3)C2] | 1.5 | 695.383 | 696.5 | LC method A |
| Compound 223 (diasteromer 1), hydrochloride salt | 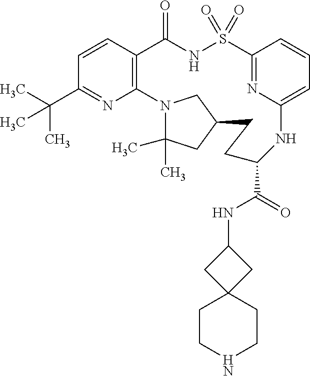 [SMILES: CC(C)(C)c1ccc2c(n1)N1C[C@@H](CC[C@@H](C(=O)NC3CC4(CCNCC4)C3)Nc3cccc(n3)S(=O)(=O)NC2=O)CC1(C)C] | 1.49 | 637.341 | 638.2 | LC method A |
| Compound 227 (diasteromer 1) | 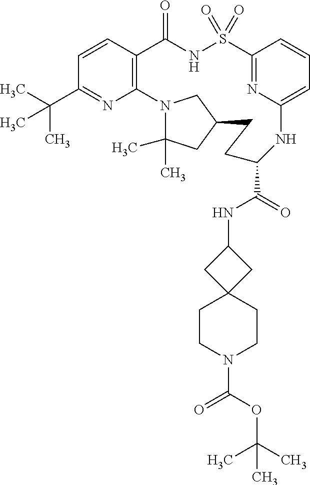 [SMILES: CC(C)(C)OC(=O)N1CCC2(CC1)CC(NC(=O)[C@@H]1CC[C@@H]3CN(c4nc(C(C)(C)C)ccc4C(=O)NS(=O)(=O)c4cccc(n4)N1)C(C)(C)C3)C2] | 2.24 | 737.393 | 738.2 | LC method A |

### Example 70: Preparation of (14S)-8-tert-Butyl-12,12,18-trimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione, Compound 261 (diastereomer 2)

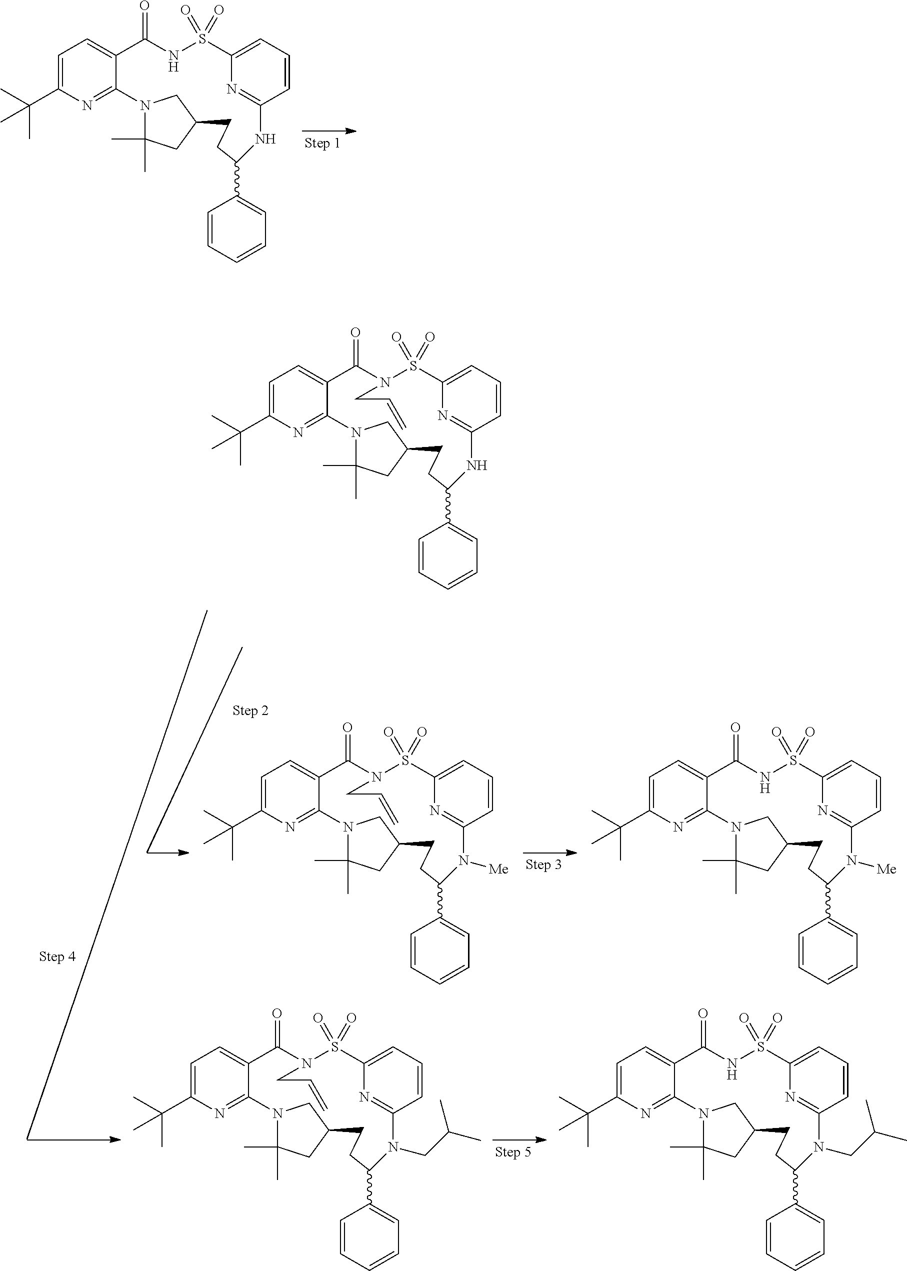 [SMILES: CC(C)(C)c1ccc2c(n1)N1C[C@@H](CCC(c3ccccc3)Nc3cccc(n3)S(=O)(=O)NC2=O)CC1(C)C ; C=CCN1C(=O)c2ccc(C(C)(C)C)nc2N2C[C@@H](CCC(c3ccccc3)Nc3cccc(n3)S1(=O)=O)CC2(C)C ; C=CCN1C(=O)c2ccc(C(C)(C)C)nc2N2C[C@@H](CCC(c3ccccc3)N(C)c3cccc(n3)S1(=O)=O)CC2(C)C ; CN1c2cccc(n2)S(=O)(=O)NC(=O)c2ccc(C(C)(C)C)nc2N2C[C@@H](CCC1c1ccccc1)CC2(C)C ; C=CCN1C(=O)c2ccc(C(C)(C)C)nc2N2C[C@@H](CCC(c3ccccc3)N(CC(C)C)c3cccc(n3)S1(=O)=O)CC2(C)C ; CC(C)CN1c2cccc(n2)S(=O)(=O)NC(=O)c2ccc(C(C)(C)C)nc2N2C[C@@H](CCC1c1ccccc1)CC2(C)C]

### Step 1: (14S)-8-tert-Butyl-12,12-dimethyl-17-phenyl-3-(prop-2-en-1-yl)-2λ⁶-thia-3,9,11,18,23-pentaazatetracyclo[17.3.1.111,14.05,10]tetracosa-1(22),5,7,9,19(23),20-hexaene-2,2,4-trione, diastereomer 2

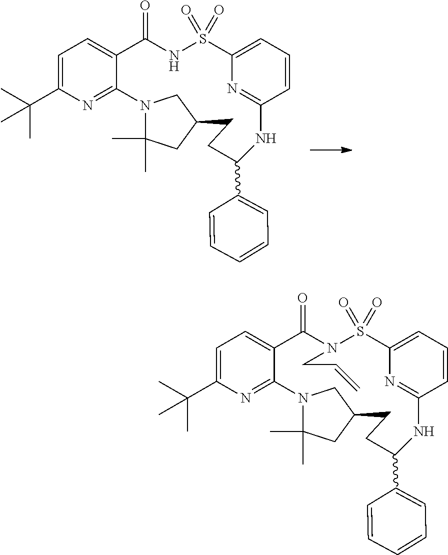 [SMILES: CC(C)(C)c1ccc2c(n1)N1C[C@@H](CCC(c3ccccc3)Nc3cccc(n3)S(=O)(=O)NC2=O)CC1(C)C ; C=CCN1C(=O)c2ccc(C(C)(C)C)nc2N2C[C@@H](CCC(c3ccccc3)Nc3cccc(n3)S1(=O)=O)CC2(C)C]

To a microwave vial was added potassium carbonate (35 mg, 0.2532 mmol), a solution of (14S)-8-tert-butyl-12,12-dimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione Compound 294 (diastereomer 2), (34 mg, 0.06208 mmol) in DMF (1 mL) and allyl bromide (6.4 μL, 0.07396 mmol). The reaction was allowed to stir overnight at rt. Additional allyl bromide (1.6 μL, 0.01849 mmol) was added and the reaction was allowed to stir for 4 h. The reaction was filtered and purified via HPLC (30-99%) ACN:H₂O with an HCl modifier to provide (14S)-8-tert-butyl-12,12-dimethyl-17-phenyl-3-(prop-2-en-1-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 2, 34 mg, 93%). ESI-MS m/z calc. 587.293, found 588.5 (M+1)⁺; Retention time: 2.11 minutes (LC method G).

### Step 2: (14S)-8-tert-Butyl-12,12,18-trimethyl-17-phenyl-3-(prop-2-en-1-yl)-2λ⁶-thia-3,9,11,18,23-pentaazatetracyclo[17.3.1.111,14.05,10]tetracosa-1(22),5,7,9,19(23),20-hexaene-2,2,4-trione

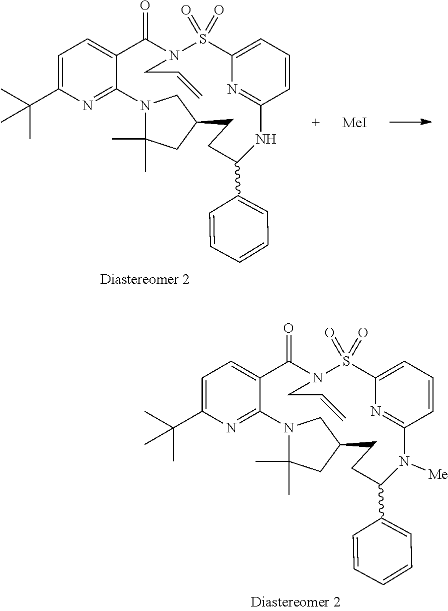 [SMILES: "MeI" ; C=CCN1C(=O)c2ccc(C(C)(C)C)nc2N2C[C@@H](CCC(c3ccccc3)Nc3cccc(n3)S1(=O)=O)CC2(C)C ; C=CCN1C(=O)c2ccc(C(C)(C)C)nc2N2C[C@@H](CCC(c3ccccc3)N(C)c3cccc(n3)S1(=O)=O)CC2(C)C]

To a vial containing (14S)-8-tert-butyl-12,12-dimethyl-17-phenyl-3-(prop-2-en-1-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 2, 34 mg, 0.05785 mmol) was added DMF (1 mL) and sodium hydride (3.0 mg of 60% w/w, 0.07501 mmol) followed by iodomethane (3.6 μL, 0.05783 mmol). Additional iodomethane (1.5 μL, 0.02409 mmol) was added and the reaction was allowed to stir overnight. The reaction was filtered and purified via HPLC 50%-99% ACN:H₂O with an HCl modifier. (14S)-8-tert-butyl-12,12,18-trimethyl-17-phenyl-3-(prop-2-en-1-yl)-2λ⁶-thia-3,9,11,18,23-pentaazatetracyclo[17.3.1.111,14.05,10]tetracosa-1(22),5,7,9,19(23),20-hexaene-2,2,4-trione was isolated as a solid (diastereomer 2, 12.6 mg, 36%). ESI-MS m/z calc. 601.30865, found 602.4 (M+1)⁺; Retention time: 2.31 minutes (LC method G).

### Step 3: (14S)-8-tert-Butyl-12,12,18-trimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione, Compound 261 (diastereomer 2)

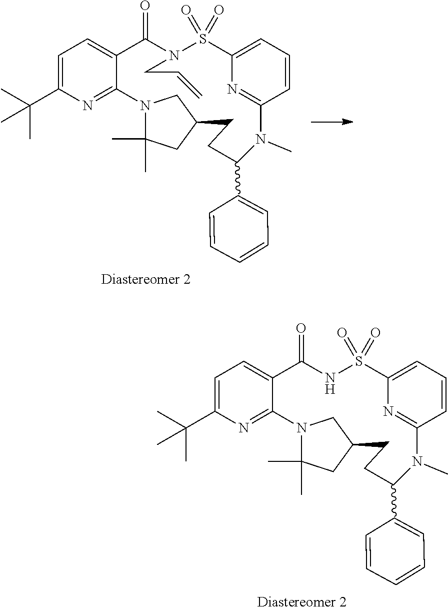

To a vial containing (14S)-8-tert-butyl-12,12,18-trimethyl-17-phenyl-3-(prop-2-en-1-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 2, 12.6 mg, 0.02094 mmol) was added 1,2-dichloroethane (0.5 mL), and tetrakis(triphenylphosphane)palladium(0) (24.2 mg, 0.02094 mmol). The reaction was allowed to stir for 30 minutes and then sodium benzenesulfonate (3.9 mg, 0.1696 mmol) was added. After 20 minutes, the reaction was filtered and purified via HPLC 30%-99% ACN:H₂O with an HCl modifier. (14S)-8-tert-butyl-12,12,18-trimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione was isolated as a yellow solid (diastereomer 2, 7.1 mg, 60%). ESI-MS m/z calc. 561.27734, found 562.4 (M+1)⁺; Retention time: 1.8 minutes (LC method G).

### Step 4: (14S)-8-tert-Butyl-12,12-dimethyl-18-(2-methylpropyl)-17-phenyl-3-(prop-2-en-1-yl)-2λ⁶-thia-3,9,11,18,23-pentaazatetracyclo[17.3.1.111,14.05,10]tetracosa-1(22),5,7,9,19(23),20-hexaene-2,2,4-trione diastereomer 2

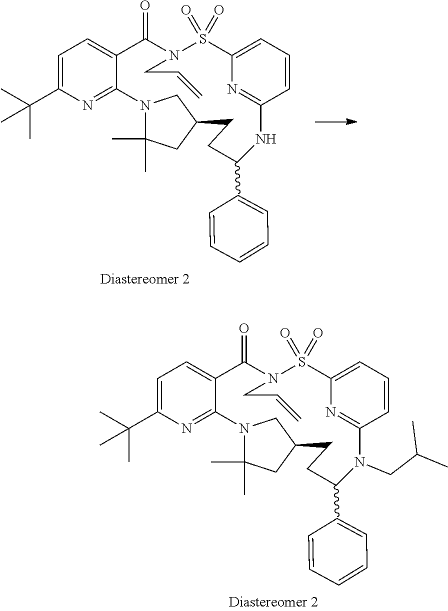 [SMILES: C=CCN1C(=O)c2ccc(C(C)(C)C)nc2N2C[C@@H](CCC(c3ccccc3)Nc3cccc(n3)S1(=O)=O)CC2(C)C ; C=CCN1C(=O)c2ccc(C(C)(C)C)nc2N2C[C@@H](CCC(c3ccccc3)N(CC(C)C)c3cccc(n3)S1(=O)=O)CC2(C)C]

To a microwave vial was added (14S)-8-tert-butyl-12,12-dimethyl-17-phenyl-3-(prop-2-en-1-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 2, 19.5 mg, 0.03318 mmol) and 1-bromo-2-methyl-propane (110 μL, 1.012 mmol). The reaction was sonicated until it went into solution. Sodium hydride (2.7 mg of 60% w/w, 0.06751 mmol) was added followed by DMF (0.2 mL) and the reaction was allowed to stir at rt overnight. The reaction was quenched with methanol and purified via HPLC 50%-99% ACN:H₂O with an HCl modifier. (14S)-8-tert-Butyl-12,12-dimethyl-18-(2-methylpropyl)-17-phenyl-3-(prop-2-en-1-yl)-2λ⁶-thia-3,9,11,18,23-pentaazatetracyclo[17.3.1.111,14.05,10]tetracosa-1(22),5,7,9,19(23),20-hexaene-2,2,4-trione was isolated as a solid (diastereomer 2, 8.2 mg, 38%). ESI-MS m/z calc. 643.3556, found 644.5 (M+1)⁺; Retention time: 2.54 minutes (LC method G).

### Step 5: (14S)-8-tert-Butyl-12,12-dimethyl-18-(2-methylpropyl)-17-phenyl-2λ⁶-thia-3,9,11,18,23-pentaazatetracyclo[17.3.1.111,14.05,10]tetracosa-1(22),5,7,9,19(23),20-hexaene-2,2,4-trione, Compound 270 (diastereomer 2)

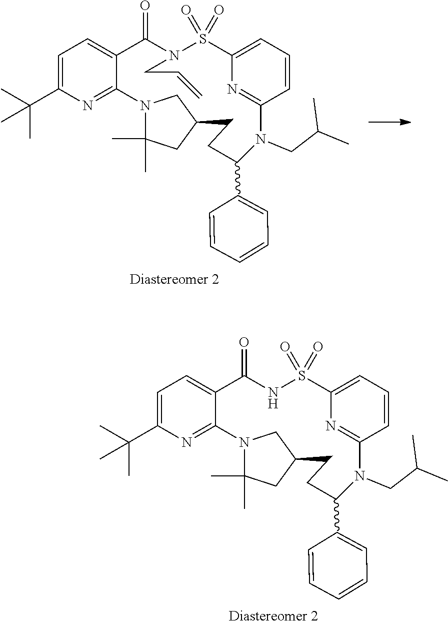

To a test tube containing (14S)-8-tert-butyl-12,12-dimethyl-18-(2-methylpropyl)-17-phenyl-3-(prop-2-en-1-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 2, 8.2 mg, 0.01274 mmol) was added 1,2-dichloroethane (0.5 mL). Pd(PPh₃)₄ (14.8 mg, 0.01281 mmol) was added and the reaction was stirred for 5 minutes while the reaction turned to a dark red. Sodium benzenesulfonate (2.4 mg, 0.1044 mmol) was added and the reaction was allowed to stir at rt for 1 h. The reaction was evaporated, dissolved in DMF, and filtered. The crude reaction mixture purified via HPLC 50%-99% ACN:H₂O with an HCl modifier. (14S)-8-tert-butyl-12,12-dimethyl-18-(2-methylpropyl)-17-phenyl-2λ⁶-thia-3,9,11,18,23-pentaazatetracyclo[17.3.1.111,14.05,10]tetracosa-1(22),5,7,9,19(23),20-hexaene-2,2,4-trione was isolated as a solid (diastereomer 2, 5.3 mg, 69%). ESI-MS m/z calc. 603.32434, found 604.4 (M+1)⁺; Retention time: 2.23 minutes (LC method G).

The compounds in the following table were prepared in a manner analogous to that described above, using (14S)-8-tert-butyl-12,12-dimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione, Compound 295 (diastereomer 1), as a starting material:

| | | LCMS | | | |
|---|---|---|---|---|---|
| | | Retention | | | |
| Compound | | Time | Exact | | LCMS |
| Number | Structure | (min) | Mass | M + 1 | Method |
| Compound 262 (diastereromer 1) | 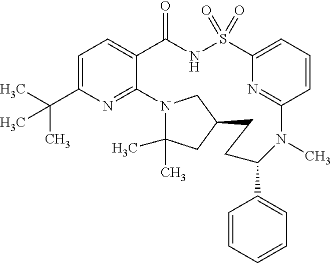 [SMILES: CN1c2cccc(n2)S(=O)(=O)NC(=O)c2ccc(C(C)(C)C)nc2N2C[C@@H](CC[C@H]1c1ccccc1)CC2(C)C] | 1.91 | 561.277 | 562.4 | LC method G |
| Compound 271 (diastereomer 1) | 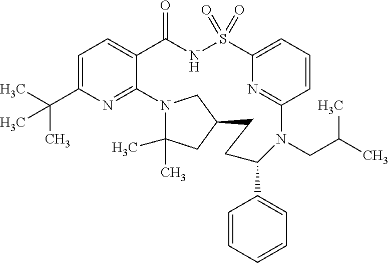 [SMILES: CC(C)CN1c2cccc(n2)S(=O)(=O)NC(=O)c2ccc(C(C)(C)C)nc2N2C[C@@H](CC[C@H]1c1ccccc1)CC2(C)C] | 2.21 | 603.324 | 604.5 | LC method G |

### Example 71: Preparation of (14S)-12,12-dimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione, Compound 289 (diastereomer 1) and Compound 288 (diastereomer 2)

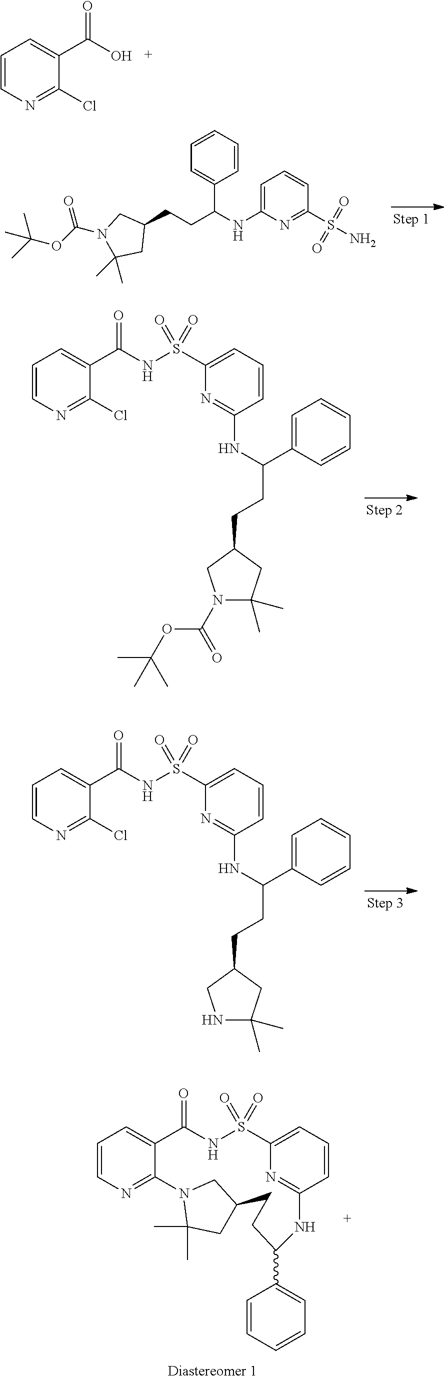

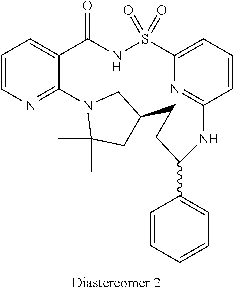 [SMILES: CC1(C)C[C@@H]2CCC(c3ccccc3)Nc3cccc(n3)S(=O)(=O)NC(=O)c3cccnc3N1C2]

### Step 1: tert-Butyl (4S)-4-[3-[[6-[(2-chloropyridine-3-carbonyl)sulfamoyl]-2-pyridyl]amino]-3-phenyl-propyl]-2,2-dimethyl-pyrrolidine-1-carboxylate

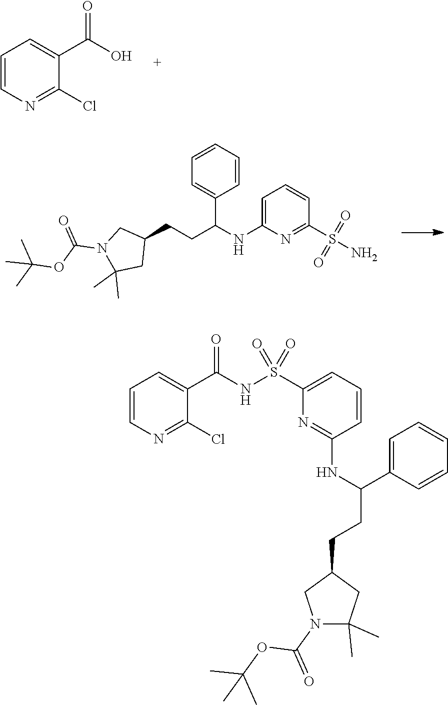 [SMILES: O=C(O)c1cccnc1Cl ; CC(C)(C)OC(=O)N1C[C@@H](CCC(Nc2cccc(S(N)(=O)=O)n2)c2ccccc2)CC1(C)C ; CC(C)(C)OC(=O)N1C[C@@H](CCC(Nc2cccc(S(=O)(=O)NC(=O)c3cccnc3Cl)n2)c2ccccc2)CC1(C)C]

To a solution of the 2-chloropyridine-3-carboxylic acid (126.0 mg, 0.8 mmol) in THF (1.008 mL) was added CDI (162 mg, 0.9991 mmol) (recrystallized from THF) and the mixture was stirred at rt for 1 h then tert-butyl (4S)-2,2-dimethyl-4-[3-phenyl-3-[(6-sulfamoyl-2-pyridyl)amino]propyl]pyrrolidine-1-carboxylate (408.9 mg, 0.8368 mmol) was added followed by DBU (376 μL, 2.512 mmol) and the resulting mixture was stirred for 16 h at room temperature. Concentrated to remove the THF then diluted with DMSO, filtered and purified using a reverse phase HPLC-MS method using a Luna C₁₈ column (75×30 mm, 5 μm particle size) sold by Phenomenex (pn: 00C-4252-U0-AX), and a dual gradient run from 1-99% mobile phase B over 15.0 minutes (mobile phase A=H₂O (5 mM HCl), mobile phase B=acetonitrile, flow rate=50 mL/min, injection volume=950 μL and column temperature=25° C.) giving tert-butyl (4S)-4-[3-[[6-[(2-chloropyridine-3-carbonyl)sulfamoyl]-2-pyridyl]amino]-3-phenyl-propyl]-2,2-dimethyl-pyrrolidine-1-carboxylate (280.8 mg, 56%). ESI-MS m/z calc. 627.2282, found 628.2 (M+1)⁺; Retention time: 0.77 minutes (LC method D).

### Step 2: 2-Chloro-N-[[6-[[3-[(3S)-5,5-dimethylpyrrolidin-3-yl]-1-phenyl-propyl]amino]-2-pyridyl]sulfonyl]pyridine-3-carboxamide

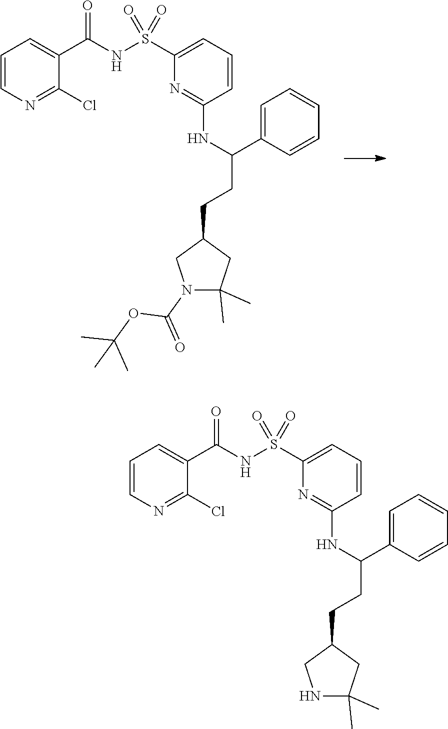 [SMILES: CC(C)(C)OC(=O)N1C[C@@H](CCC(Nc2cccc(S(=O)(=O)NC(=O)c3cccnc3Cl)n2)c2ccccc2)CC1(C)C ; CC1(C)C[C@H](CCC(Nc2cccc(S(=O)(=O)NC(=O)c3cccnc3Cl)n2)c2ccccc2)CN1]

tert-Butyl (4S)-4-[3-[[6-[(2-chloropyridine-3-carbonyl)sulfamoyl]-2-pyridyl]amino]-3-phenyl-propyl]-2,2-dimethyl-pyrrolidine-1-carboxylate (280.7 mg, 0.4468 mmol) was dissolved in DCM (1.225 mL) and to the mixture was added TFA (2.039 g, 17.88 mmol) and the mixture was stirred at room temperature for 2 h. Concentrated mixture to dryness under reduced pressure, added 1 mL of toluene and removed by rotary evaporation (45° C. water bath). Again added 1 mL of toluene and removed by rotary evaporation (45° C. water bath) then dried on the high vacuum giving 2-chloro-N-[[6-[[3-[(3S)-5,5-dimethylpyrrolidin-3-yl]-1-phenyl-propyl]amino]-2-pyridyl]sulfonyl]pyridine-3-carboxamide (Trifluoroacetate salt) (287 mg, 100%). ESI-MS m/z calc. 527.1758, found 528.4 (M+1)⁺; Retention time: 0.44 minutes as a white solid (LC method D).

### Step 3: (14S)-12,12-Dimethyl-17-phenyl-2λ⁶-thia-3,9,11,18,23-pentaazatetracyclo [17.3.1.111,14.05,10]tetracosa-1(22),5,7,9,19(23),20-hexaene-2,2,4-trione, Compound 289 (diastereomer 1), and (14S)-12,12-dimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione, Compound 288 (diastereomer 2)

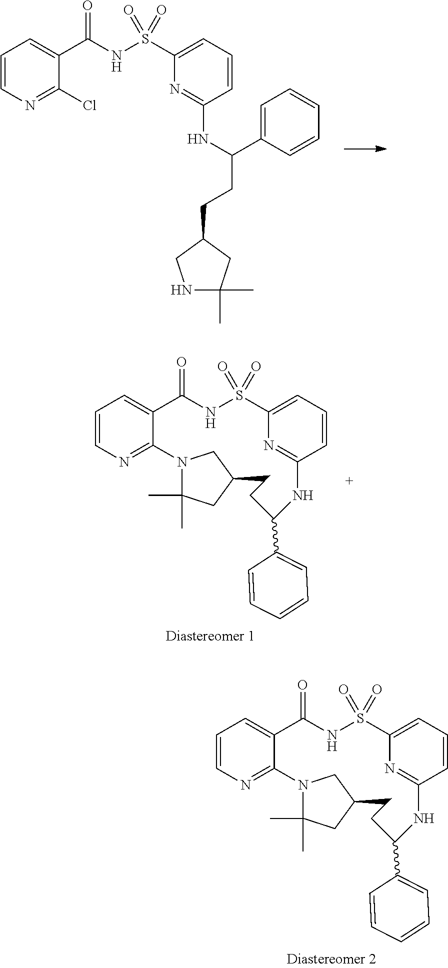

To a solution of 2-chloro-N-[[6-[[3-[(3S)-5,5-dimethylpyrrolidin-3-yl]-1-phenyl-propyl]amino]-2-pyridyl]sulfonyl]pyridine-3-carboxamide (Trifluoroacetate salt) (287 mg, 0.4470 mmol) in NMP (17 mL) was added potassium carbonate (432.6 mg, 3.130 mmol). The mixture was purged with nitrogen for 5 min. The mixture was heated at 165° C. for 2 days. The mixture was cooled to room temperature, diluted with 1N HCl and extracted with EtOAc (2×). The organic phases were combined, dried (sodium sulfate), filtered and concentrated to a brown oil which was filtered and purified using a reverse phase HPLC-MS method using a Luna C₁₈ column (75×30 mm, 5 μm particle size) sold by Phenomenex (pn: 00C-4252-U0-AX), and a dual gradient run from 30-70% mobile phase B over 30.0 minutes (mobile phase A=H₂O (5 mM HCl), mobile phase B=acetonitrile, flow rate=50 mL/min, injection volume=950 μL and column temperature=25° C.) giving two isomers, both as white solids:

First to elute, diastereomer 1: (14S)-12,12-dimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione (13.70 mg, 12%). ¹H NMR (500 MHz, DMSO-d₆) δ 12.55 (s, 1H), 8.17 (s, 1H), 7.74 (s, 1H), 7.63-7.57 (m, 1H), 7.51 (d, J=7.6 Hz, 2H), 7.33 (s, 2H), 7.28-7.16 (m, 2H), 6.80 (d, J=8.5 Hz, 1H), 6.66 (s, 1H), 6.16 (s, 1H), 4.82 (s, 1H), 3.21 (s, 1H), 3.14 (s, 1H), 2.34 (s, 1H), 1.87 (d, J=12.0 Hz, 2H), 1.71 (s, 2H), 1.58 (s, 3H), 1.55 (s, 1H), 1.52 (s, 3H), 1.17 (s, 1H). ESI-MS m/z calc. 491.1991, found 492.0 (M+1)⁺; Retention time: 1.72 minutes (LC method Q).

Second to elute, diastereomer 2: (14S)-12,12-dimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione (13 mg, 12%). ¹H NMR (500 MHz, DMSO-d₆) δ 12.62 (s, 1H), 8.17 (dd, J=4.7, 1.9 Hz, 1H), 7.74-7.67 (m, 2H), 7.62 (t, J=7.8 Hz, 1H), 7.42 (d, J=7.6 Hz, 2H), 7.30 (t, J=7.5 Hz, 2H), 7.20 (t, J=7.0 Hz, 1H), 7.07 (d, J=7.2 Hz, 1H), 6.82 (d, J=8.5 Hz, 1H), 6.62 (dd, J=7.6, 4.7 Hz, 1H), 5.20 (t, J=10.9 Hz, 1H), 3.25 (t, J 8.7 Hz, 1H), 2.77 (t, J=10.5 Hz, 1H), 2.26 (s, 1H), 1.94 (d, J=5.5 Hz, 1H), 1.85 (dd, J 11.9, 5.2 Hz, 1H), 1.72 (d, J=13.3 Hz, 2H), 1.62 (s, 3H), 1.58 (d, J=12.2 Hz, 1H), 1.48 (s, 3H), 1.45 (s, 1H). ESI-MS m/z calc. 491.1991, found 492.0 (M+1)⁺; Retention time: 1.68 minutes (LC method Q).

### Example 72: Preparation of (18S)-4-tert-Butyl-20,20-dimethyl-15-phenyl-10λ⁶-thia-1,3,9,14,22-pentaazatetracyclo[16.2.1.111,14.02,7]docosa-2,4,6,11(22),12-pentaene-8,10,10-trione, Compound 275 (diastereomer 1) and Compound 274 (diastereomer 2)

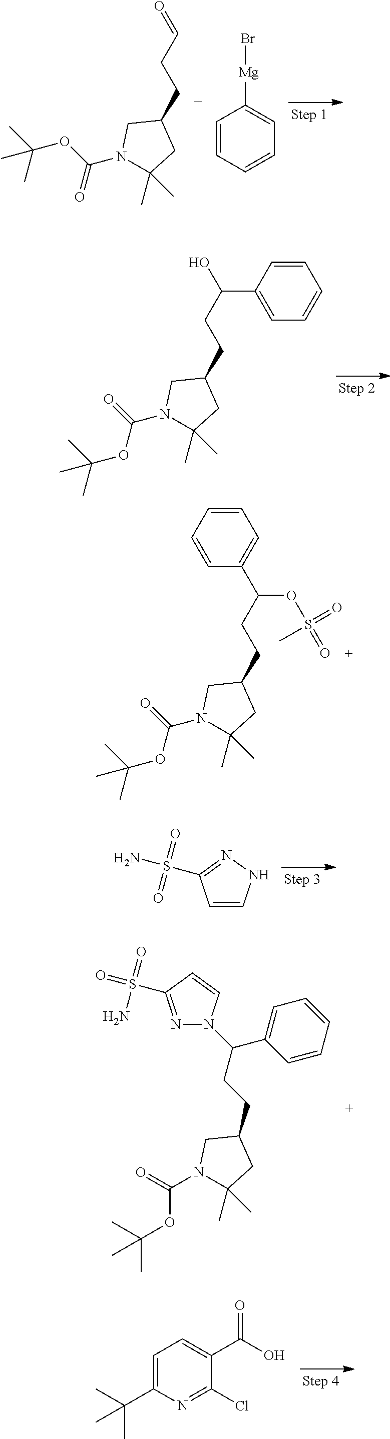

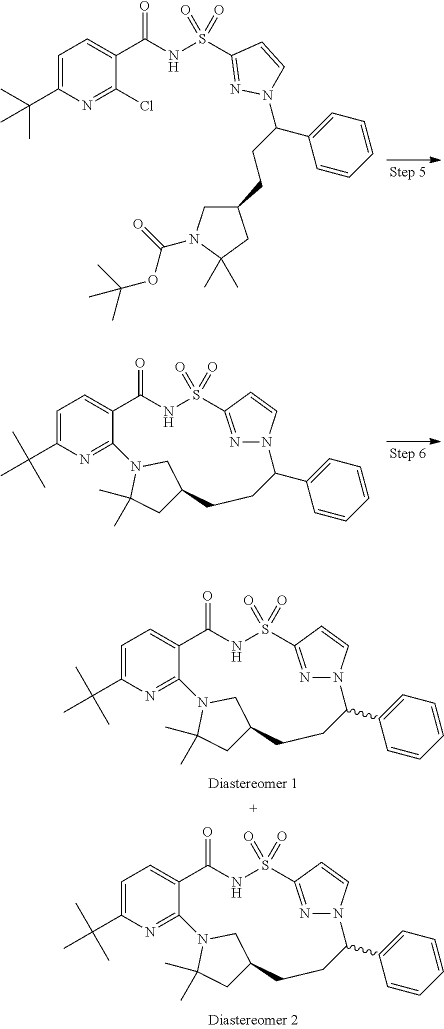 [SMILES: CC(C)(C)OC(=O)N1C[C@@H](CCC(c2ccccc2)n2ccc(S(=O)(=O)NC(=O)c3ccc(C(C)(C)C)nc3Cl)n2)CC1(C)C ; CC(C)(C)c1ccc2c(n1)N1C[C@@H](CCC(c3ccccc3)n3ccc(n3)S(=O)(=O)NC2=O)CC1(C)C ; CC(C)(C)c1ccc2c(n1)N1C[C@@H](CCC(c3ccccc3)n3ccc(n3)S(=O)(=O)NC2=O)CC1(C)C ; CC(C)(C)c1ccc2c(n1)N1C[C@@H](CCC(c3ccccc3)n3ccc(n3)S(=O)(=O)NC2=O)CC1(C)C]

### Step 1: tert-Butyl (4S)-4-(3-hydroxy-3-phenyl-propyl)-2,2-dimethyl-pyrrolidine-1-carboxylate

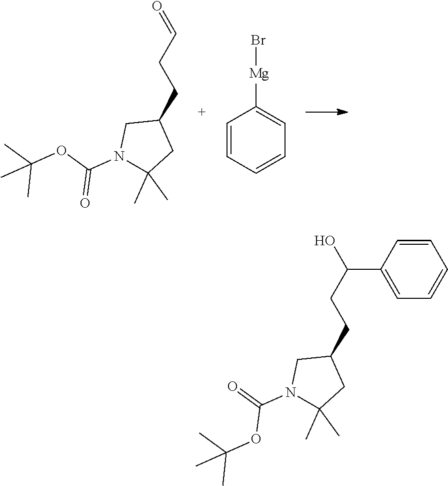

To a solution of tert-butyl (4S)-2,2-dimethyl-4-(3-oxopropyl)pyrrolidine-1-carboxylate (650 mg, 2.545 mmol) in dry THF (5 mL) at 0° C. under nitrogen atmosphere was added bromo(phenyl)magnesium (3.1 mL of 1.0 M in THF, 3.100 mmol) and the reaction mixture was stirred for 2 h while slowly warming up to rt. The reaction was quenched with a saturated ammonium chloride solution and extracted with ethyl acetate. The combined organics were washed with brine, dried over sodium sulfate and evaporated. The resultant brown residue was purified by silica gel column chromatography using a shallow gradient of 100% hexanes to 100% EtOAc to afford tert-butyl (4S)-4-(3-hydroxy-3-phenyl-propyl)-2,2-dimethyl-pyrrolidine-1-carboxylate (614 mg, 72%) as a colorless oil. ESI-MS m/z calc. 333.2304, found 334.38 (M+1)⁺; Retention time: 0.76 minutes (LC method D).

### Step 2: tert-Butyl (4S)-2,2-dimethyl-4-(3-methylsulfonyloxy-3-phenyl-propyl)pyrrolidine-1-carboxylate

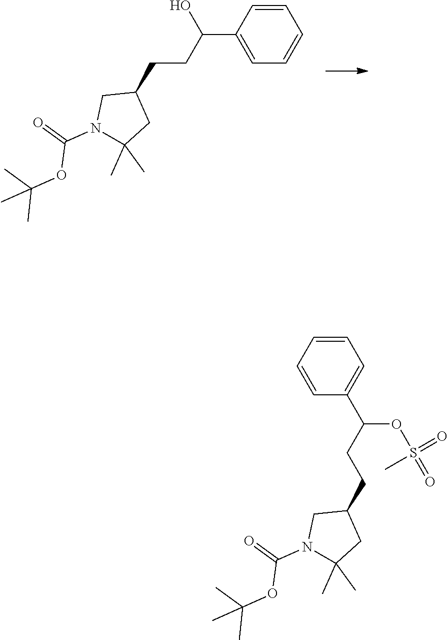 [SMILES: CC(C)(C)OC(=O)N1C[C@@H](CCC(O)c2ccccc2)CC1(C)C ; CC(C)(C)OC(=O)N1C[C@@H](CCC(OS(C)(=O)=O)c2ccccc2)CC1(C)C]

To a solution of tert-butyl (4S)-4-(3-hydroxy-3-phenyl-propyl)-2,2-dimethyl-pyrrolidine-1-carboxylate (600 mg, 1.799 mmol) in dichloromethane (5 mL), and triethylamine (760 μL, 5.453 mmol) was added methanesulfonyl chloride (170 μL, 2.196 mmol) slowly dropwise at 0° C. and the reaction was allowed to warm up to rt and stir for 60 minutes. The reaction was quenched with brine and extracted with DCM. The organic layer was dried over sodium sulfate and evaporated to afford tert-butyl (4S)-2,2-dimethyl-4-(3-methylsulfonyloxy-3-phenyl-propyl)pyrrolidine-1-carboxylate (740 mg, 40%) which was used as is for the next reaction as is. ESI-MS m/z calc. 411.20795, found 348.4 (M+1)⁺; Retention time: 0.69 minutes (LC method D).

### Step 3: tert-Butyl (4S)-2,2-dimethyl-4-[3-phenyl-3-(3-sulfamoylpyrazol-1-yl)propyl]pyrrolidine-1-carboxylate, and tert-butyl (4S)-2,2-dimethyl-4-[3-phenyl-3-(5-sulfamoylpyrazol-1-yl)propyl]pyrrolidine-1-carboxylate

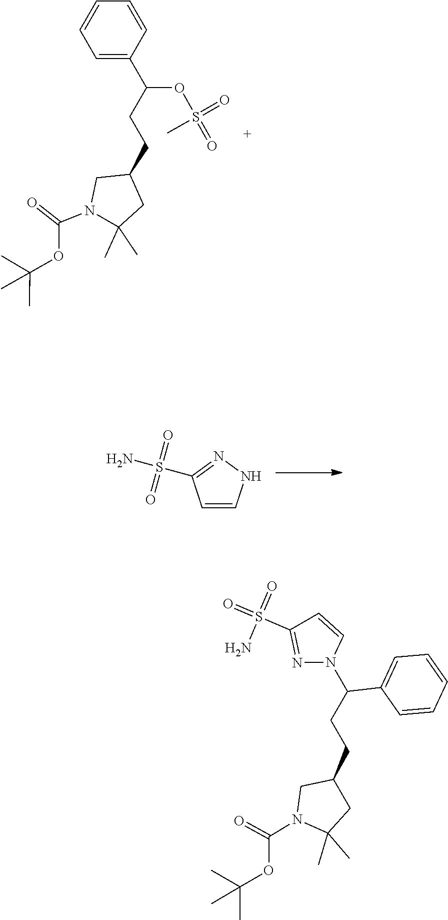 [SMILES: CC(C)(C)OC(=O)N1C[C@@H](CCC(OS(C)(=O)=O)c2ccccc2)CC1(C)C ; NS(=O)(=O)c1cc[nH]n1 ; CC(C)(C)OC(=O)N1C[C@@H](CCC(c2ccccc2)n2ccc(S(N)(=O)=O)n2)CC1(C)C]

To a solution of tert-butyl (4S)-2,2-dimethyl-4-(3-methylsulfonyloxy-3-phenyl-propyl)pyrrolidine-1-carboxylate (700 mg, 1.701 mmol) in DMF (5 mL) was added 1H-pyrazole-3-sulfonamide (757 mg, 5.144 mmol) and cesium carbonate (2.8 g, 8.594 mmol). The reaction mixture was heated at 80° C. for 24 h. To the reaction mixture, water was added and the mixture was extracted with ethylacetate. The combined organic layers were washed with brine, dried over anhydrous sodium sulphate and concentrated. The resultant brown residue was filtered through a Whatman filter disc (puradisc 25 TF) and the filtrate was purified by a reverse phase HPLC-MS method using a dual gradient run of 30-99% mobile phase B over 15.0 minutes (mobile phase A=H₂O (5 mM HCl), mobile phase B=CH₃CN. Flow rate=50 mL/min, injection volume=950 μL, and column temperature=25° C. to afford the desired tert-butyl (4S)-2,2-dimethyl-4-[3-phenyl-3-(3-sulfamoylpyrazol-1-yl)propyl]pyrrolidine-1-carboxylate (184 mg, 23%). ¹H NMR (500 MHz, DMSO-d₆) δ 8.09 (d, J=2.4 Hz, 1H), 7.47-7.26 (m, 7H), 6.62 (d, J=2.4 Hz, 1H), 5.50 (q, J=7.4, 6.8 Hz, 1H), 3.56 (dt, J=19.9, 9.0 Hz, 1H), 2.86-2.68 (m, 1H), 2.39-2.26 (m, 1H), 2.19-2.10 (m, 2H), 1.88 (ddd, J=17.1, 11.9, 6.0 Hz, 1H), 1.36 (dd, J=25.2, 14.1 Hz, 13H), 1.24 (s, 3H), 1.23-1.10 (m, 2H). ESI-MS m/z calc. 462.23007, found 463.4 (M+1)⁺; Retention time: 0.48 minutes (LC method I).

### Step 4: tert-Butyl (4S)-4-[3-[3-[(6-tert-butyl-2-chloro-pyridine-3-carbonyl)sulfamoyl]pyrazol-1-yl]-3-phenyl-propyl]-2,2-dimethyl-pyrrolidine-1-carboxylate

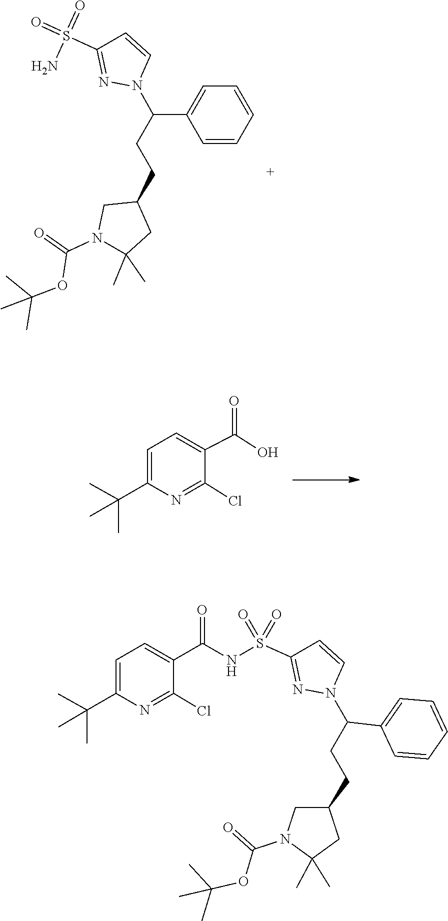 [SMILES: CC(C)(C)OC(=O)N1C[C@@H](CCC(c2ccccc2)n2ccc(S(N)(=O)=O)n2)CC1(C)C ; CC(C)(C)c1ccc(C(=O)O)c(Cl)n1 ; CC(C)(C)OC(=O)N1C[C@@H](CCC(c2ccccc2)n2ccc(S(=O)(=O)NC(=O)c3ccc(C(C)(C)C)nc3Cl)n2)CC1(C)C]

To a solution of the 6-tert-butyl-2-chloro-pyridine-3-carboxylic acid (126 mg, 0.5897 mmol) in THF (2 mL) was added CDI (98 mg, 0.6044 mmol) and the mixture was stirred at rt for 1 h then tert-butyl (4S)-2,2-dimethyl-4-[3-phenyl-3-(3-sulfamoylpyrazol-1-yl)propyl]pyrrolidine-1-carboxylate (180 mg, 0.3891 mmol) was added followed by DBU (200 μL, 1.337 mmol) and the resulting mixture was stirred for 16 h at room temperature. Concentrated to remove the THF then diluted with DMSO, filtered and purified using a reverse phase HPLC-MS method using a dual gradient run of 30-99% mobile phase B over 15.0 minutes (mobile phase A=H₂O (5 mM HCl), mobile phase B=acetonitrile to afford as a mixture of diastereomers tert-butyl (4S)-4-[3-[3-[(6-tert-butyl-2-chloro-pyridine-3-carbonyl)sulfamoyl]pyrazol-1-yl]-3-phenyl-propyl]-2,2-dimethyl-pyrrolidine-1-carboxylate (98 mg, 38%). ESI-MS m/z calc. 657.27515, found 658.5 (M+1)⁺; Retention time: 0.7 minutes (LC method I).

### Step 5: (185)-4-tert-Butyl-20,20-dimethyl-15-phenyl-10λ⁶-thia-1,3,9,14,22-pentaazatetracyclo[16.2.1.111,14.02,7]docosa-2,4,6,11(22),12-pentaene-8,10,10-trione

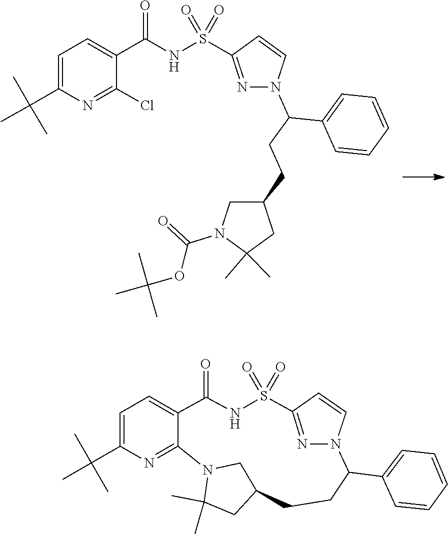 [SMILES: CC(C)(C)OC(=O)N1C[C@@H](CCC(c2ccccc2)n2ccc(S(=O)(=O)NC(=O)c3ccc(C(C)(C)C)nc3Cl)n2)CC1(C)C ; CC(C)(C)c1ccc2c(n1)N1C[C@@H](CCC(c3ccccc3)n3ccc(n3)S(=O)(=O)NC2=O)CC1(C)C]

A solution of tert-butyl (4S)-4-[3-[3-[(6-tert-butyl-2-chloro-pyridine-3-carbonyl)sulfamoyl]pyrazol-1-yl]-3-phenyl-propyl]-2,2-dimethyl-pyrrolidine-1-carboxylate (98 mg, 0.1489 mmol) in DCM (500 μL) and TFA (100 μL, 1.298 mmol) was stirred at rt for 2 h. The solvent was evaporated. The material was dissolved in NMP (2 mL) and potassium carbonate (322 mg, 2.330 mmol) was added. The mixture was purged with nitrogen for 5 min. The mixture was heated at 140° C. for 2 days and at 160° C. for 3 more days. The reaction mixture was filtered through Whatman filter disc (puradisc 25 TF) and filtrate was purified by a reverse phase HPLC-MS method using a Luna C₁₈ column (75×30 mm, 5 μm particle size) sold by Phenomenex (pn: 00C-4252-U0-AX), and a dual gradient run of 30-99% mobile phase B over 15.0 minutes (mobile phase A=H₂O (5 mM HCl), mobile phase B=CH₃CN. Flow rate=50 mL/min, injection volume=950 μL, and column temperature=25° C. to afford (18S)-4-tert-butyl-20,20-dimethyl-15-phenyl-10λ⁵-thia-1,3,9,14,22-pentaazatetracyclo[16.2.1.111,14.02,7]docosa-2,4,6,11(22),12-pentaene-8,10,10-trione (10.3 mg, 13%). ¹H NMR (500 MHz, DMSO-d₆) δ 12.77 (s, 1H), 8.22 (d, J=2.4 Hz, 1H), 7.50 (d, J=7.9 Hz, 1H), 7.41-7.33 (m, 5H), 6.90 (d, J=2.3 Hz, 1H), 6.62 (d, J=8.0 Hz, 1H), 5.55 (d, J=12.3 Hz, 1H), 2.91-2.81 (m, 1H), 2.40-2.24 (m, 2H), 2.19 (s, 1H), 2.08 (t, J=11.2 Hz, 1H), 1.98 (t, J=10.1 Hz, 1H), 1.78 (dd, J=11.7, 5.4 Hz, 1H), 1.57 (s, 3H), 1.48 (s, 3H), 1.27 (s, 10H), 0.71 (td, J=12.1, 7.2 Hz, 1H). ESI-MS m/z calc. 521.24603, found 522.16 (M+1)⁺; Retention time: 1.62 minutes (LC method G).

### Step 6: (185)-4-tert-Butyl-20,20-dimethyl-15-phenyl-10λ⁶-thia-1,3,9,14,22-pentaazatetracyclo[16.2.1.111,14.02,7]docosa-2,4,6,11(22),12-pentaene-8,10,10-trione, Compound 275 (diastereomer 1), and (185)-4-tert-butyl-20,20-dimethyl-15-phenyl-10λ⁶-thia-1,3,9,14,22-pentaazatetracyclo[16.2.1.111,14.02,7]docosa-2,4,6,11(22),12-pentaene-8,10,10-trione, Compound 274 (diastereomer 2)

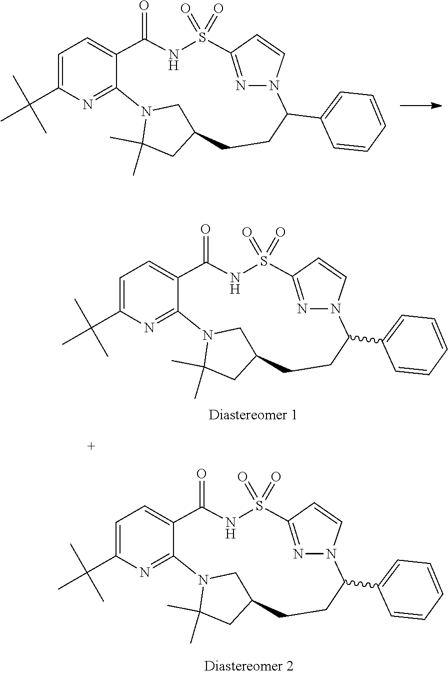 [SMILES: CC(C)(C)c1ccc2c(n1)N1C[C@@H](CCC(c3ccccc3)n3ccc(n3)S(=O)(=O)NC2=O)CC1(C)C ; CC(C)(C)c1ccc2c(n1)N1C[C@@H](CCC(c3ccccc3)n3ccc(n3)S(=O)(=O)NC2=O)CC1(C)C ; CC(C)(C)c1ccc2c(n1)N1C[C@@H](CCC(c3ccccc3)n3ccc(n3)S(=O)(=O)NC2=O)CC1(C)C]

(18S)-4-tert-Butyl-20,20-dimethyl-15-phenyl-10λ⁵-thia-1,3,9,14,22-pentaazatetracyclo[16.2.1.111,14.02,7]docosa-2,4,6,11(22),12-pentaene-8,10,10-tri one (diastereomeric mixture, 8 mg, 0.01534 mmol) was subjected to chiral separation by SFC chromatography using Phenomenex LUX-4 (250×10 mm), 5 μm particle size using 38% MeOH (No modifier), 62% CO₂ (No modifier), 65% at 10 mL/min over 14 minutes giving two isomers:

First to elute, diastereomer 1: (18S)-4-tert-Butyl-20,20-dimethyl-15-phenyl-10λ⁵-thia-1,3,9,14,22-pentaazatetracyclo[16.2.1.111,14.02,7]docosa-2,4,6,11(22),12-pentaene-8,10,10-trione (3.0 mg, 75%). ESI-MS m/z calc. 521.24603, found 522.1 (M+1)⁺; Retention time: 1.6 minutes (LC method G).

Second to elute, diastereomer 2: (18S)-4-tert-Butyl-20,20-dimethyl-15-phenyl-10λ⁵-thia-1,3,9,14,22-pentaazatetracyclo[16.2.1.111,14.02,7]docosa-2,4,6,11(22),12-pentaene-8,10,10-trione (1.5 mg, 37%) ESI-MS m/z calc. 521.24603, found 522.16 (M+1)⁺; Retention time: 1.61 minutes (LC method G).

The compounds in the following tables were prepared in a manner analogous to that described above, using methyl magnesium bromide as a starting material in step 1:

| | | LCMS | | | |
|---|---|---|---|---|---|
| | | Retention | | | |
| Compound | | Time | Exact | | LCMS |
| Number | Structure | (min) | Mass | M + 1 | Method |
| Compound 273 (diastereomer 1) | 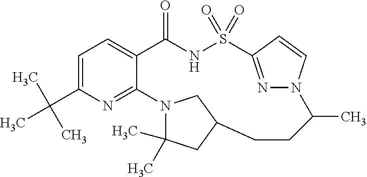 [SMILES: CC1CCC2CN(c3nc(C(C)(C)C)ccc3C(=O)NS(=O)(=O)c3ccn1n3)C(C)(C)C2] | 1.34 | 459.23 | 460.17 | LC method G |
| Compound 272 (diastereomer 2) | 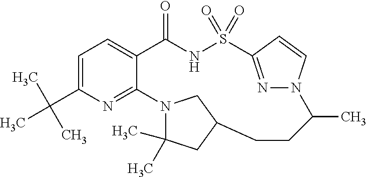 [SMILES: CC1CCC2CN(c3nc(C(C)(C)C)ccc3C(=O)NS(=O)(=O)c3ccn1n3)C(C)(C)C2] | 1.34 | 459.23 | 460.11 | LC method G |

| Compound | | |
|---|---|---|
| Number | Structure | NMR |
| Compound 273 (diastereomer 1) | 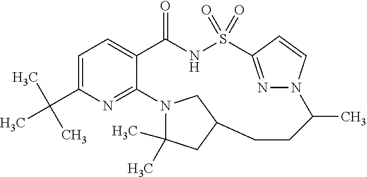 [SMILES: CC1CCC2CN(c3nc(C(C)(C)C)ccc3C(=O)NS(=O)(=O)c3ccn1n3)C(C)(C)C2] | ¹H NMR (500 MHz, DMSO-d) δ 12.65 (s, 1H), 8.02 (d, J = 2.3 Hz, 1H), 7.48 (d, J = 7.9 Hz, 1H), 6.86 (d, J = 2.2 Hz, 1H), 6.60 (d, J = 7.9 Hz, 1H), 4.41 (dd, J = 12.0, 6.4 Hz, 1H), 2.63 (t, J = 8.5 Hz, 1H), 2.11- 1.98 (m, 2H), 1.87 (q, J = 10.8 Hz, 1H), 1.79-1.64 (m, 3H), 1.55 (s, 3H), 1.51-1.47 (m, 1H), 1.45 (s, 3H), 1.37 (d, J = 6.6 Hz, 3H), 1.26 (s, 9H), 0.55 (q, J = 11.8 Hz, 1H). |

### Example 73: Preparation of 8-tert-Butyl-12,12-dimethyl-2λ⁶-thia-3,9,11,18,21,23-hexaazatetracyclo[17.3.1.111,14.05,10]tetracosa-1(22),5,7,9,19(23),20-hexaene-2,2,4-trione, Compound 253 (enantiomer 1), and Compound 252 (enantiomer 2)

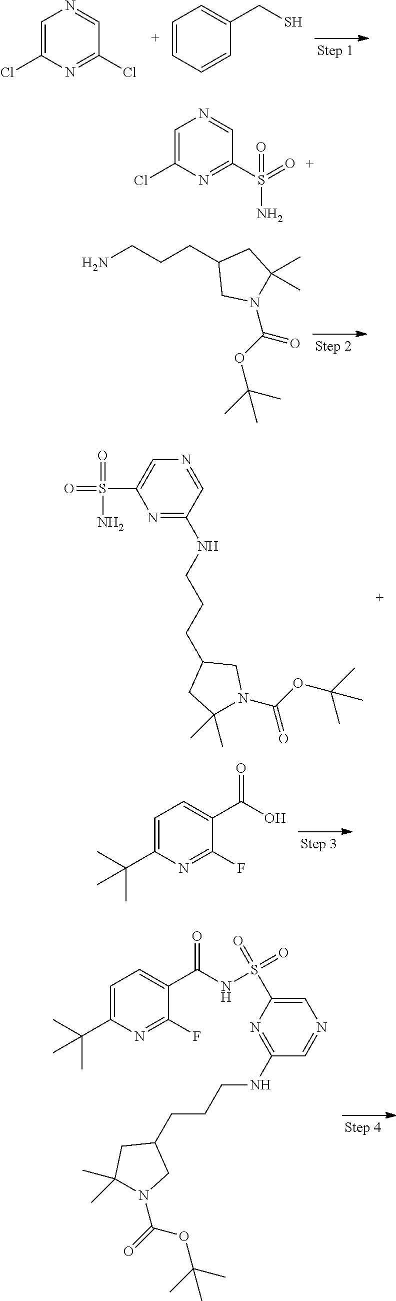

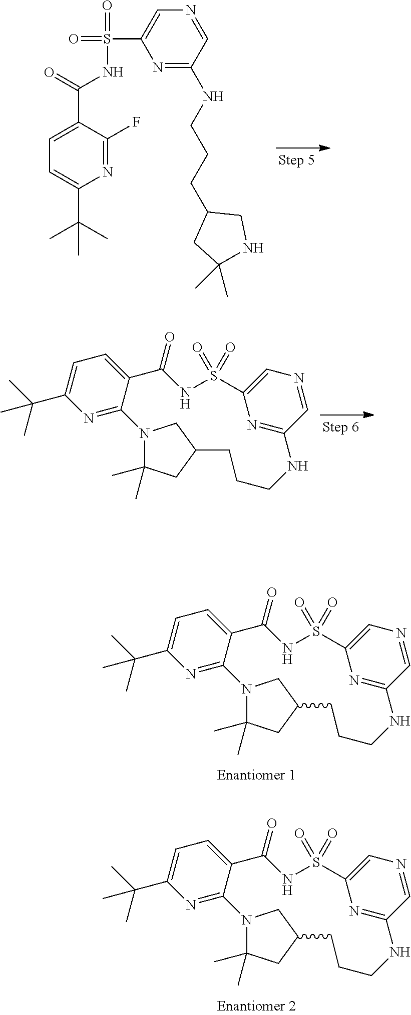 [SMILES: CC1(C)CC(CCCNc2cncc(S(=O)(=O)NC(=O)c3ccc(C(C)(C)C)nc3F)n2)CN1 ; CC(C)(C)c1ccc2c(n1)N1CC(CCCNc3cncc(n3)S(=O)(=O)NC2=O)CC1(C)C ; CC(C)(C)c1ccc2c(n1)N1CC(CCCNc3cncc(n3)S(=O)(=O)NC2=O)CC1(C)C ; CC(C)(C)c1ccc2c(n1)N1CC(CCCNc3cncc(n3)S(=O)(=O)NC2=O)CC1(C)C]

### Step 1: 6-chloropyrazine-2-sulfonamide

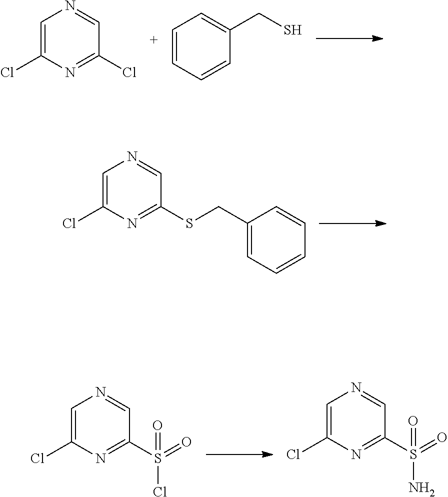

A mixture of 2,6-dichloropyrazine (7.2 g, 48 mmol, 1.0 eq), phenylmethanethiol (5.8 mL, 49 mmol, 1.0 eq), and sodium hydroxide (2.5 g, 62 mmol, 1.3 eq) in 40 mL of EtOH was stirred at rt for 6 h. The ethanol was rotary evaporated, 100 mL of water was added, and was extracted with dichloromethane (3×100 mL). The combined organic extracts were dried with MgSO₄ and filtered. The filtrate was rotary evaporated and dried under vacuum to give crude 2-(benzylthio)-6-chloropyrazine (11 g) as a light yellow oil. ¹H NMR (CDCl₃, 250 MHz): δ 8.31 (s, 1H), 8.21 (s, 1H), 7.36 (m, 5H), 4.41 (s, 2H) ppm. LC-MS: (M+H)⁺=237.1

A mixture of 2-(benzylthio)-6-chloropyrazine (6.4 g, 27 mmol, 1.0 eq) in 100 mL of chloroform and 25 mL of water was cooled to 0° C. and chlorine gas was bubbled through the solution for 5 min. A 30% aqueous sodium hydroxide trap was used for excess Cl₂. The mixture was stirred at rt for 30 min. The top aqueous layer was removed and discarded. The bottom organic layer was cooled to 0° C. and 40 mL of NH₄OH was carefully added. The reaction mixture was stirred at rt for 45 min, rotary evaporated, and purified by flash chromatography using a 120 g column and eluting with a gradient of 50-100% EtOAc/hexanes over 30 min to produce 6-chloropyrazine-2-sulfonamide (2.9 g, 56% yield) as a white solid. ¹H NMR (DMSO-d₆, 250 MHz): δ 9.08 (m, 2H), 7.97 (s, br, 2H) ppm. LC-MS: (M+H)⁺=193.4 (LC method P).

### Step 2: tert-Butyl 2,2-dimethyl-4-[3-[(6-sulfamoylpyrazin-2-yl)amino]propyl]pyrrolidine-1-carboxylate

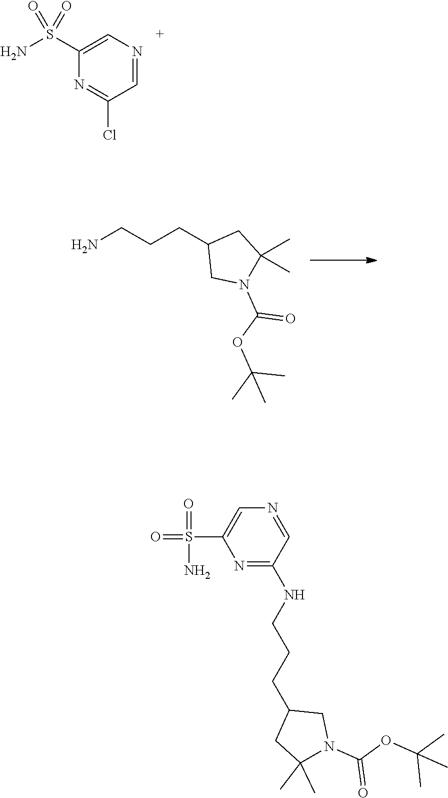 [SMILES: NS(=O)(=O)c1cncc(Cl)n1 ; CC(C)(C)OC(=O)N1CC(CCCN)CC1(C)C ; CC(C)(C)OC(=O)N1CC(CCCNc2cncc(S(N)(=O)=O)n2)CC1(C)C]

In a 20 mL vial 6-chloropyrazine-2-sulfonamide (312 mg, 1.611 mmol), tert-butyl 4-(3-aminopropyl)-2,2-dimethyl-pyrrolidine-1-carboxylate (295 mg, 1.151 mmol), DIPEA (1.0 mL, 5.741 mmol) were dissolved in n-BuOH (2.25 mL). The vial was sealed and stirred at 140° C. overnight. The reaction was cooled and poured into water (100 mL) and extracted with ethyl acetate (2×50 mL). Combined organic layers, washed with water (2×50 mL), dried (Na₂SO₄), filtered and concentrated. The orange residue was purified by silica gel chromatography (40 gram column, ELSD detection) using a gradient from 100% hexanes to 80% ethyl acetate in hexanes to afford a light yellow solid tert-butyl 2,2-dimethyl-4-[3-[(6-sulfamoylpyrazin-2-yl)amino]propyl]pyrrolidine-1-carboxylate (264 mg, 55%). ¹H NMR (400 MHz, DMSO-d₆) δ 8.06 (d, J=6.1 Hz, 2H), 7.61 (s, 1H), 7.38 (s, 2H), 3.56 (q, J=9.7 Hz, 1H), 3.31-3.28 (m, 2H), 2.89-2.72 (m, 1H), 2.24-2.03 (m, 1H), 1.90 (tt, J=13.7, 6.1 Hz, 1H), 1.56 (dt, J=15.0, 6.9 Hz, 2H), 1.45 (d, J=12.2 Hz, 1H), 1.39 (d, J=11.2 Hz, 12H), 1.34 (s, 1H), 1.29 (dd, J=13.7, 7.0 Hz, 1H), 1.25 (s, 3H). ESI-MS m/z calc. 413.2097, found 414.4 (M+1)⁺; Retention time: 1.62 minutes (LC method A).

### Step 3: tert-Butyl 4-[3-[[6-[(6-tert-butyl-2-fluoro-pyridine-3-carbonyl)sulfamoyl]pyrazin-2-yl]amino]propyl]-2,2-dimethyl-pyrrolidine-1-carboxylate

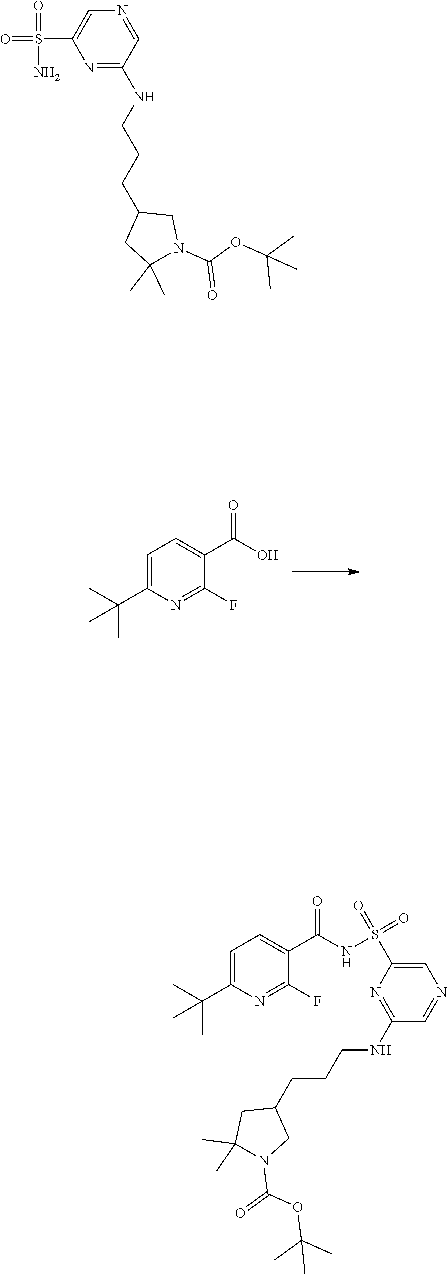

To a round bottom flask was added 6-tert-butyl-2-fluoro-pyridine-3-carboxylic acid (227.6 mg, 1.154 mmol), carbonyl diimidazole (187.1 mg, 1.154 mmol), and THF (3.7 mL). The reaction was purged with nitrogen and allowed to stir at 35° C. for 4 h. At this point, a solution of tert-butyl 2,2-dimethyl-4-[3-[(6-sulfamoylpyrazin-2-yl)amino]propyl]pyrrolidine-1-carboxylate (367 mg, 0.8875 mmol) in THF (3.7 mL) was added followed by DBU (400 μL, 2.675 mmol). The reaction was allowed to stir at rt over the weekend. By UPLC analysis, the reaction showed the desired product. The reaction was quenched with 1N citric acid and extracted with EtOAc three times. The organic layers were dried over sodium sulfate, and evaporated. The crude oil was dissolved in DMF and purified via reverse phase chromatography using 50%-99% ACN:H₂O with an HCl modifier. tert-butyl 4-[3-[[6-[(6-tert-butyl-2-fluoro-pyridine-3-carbonyl)sulfamoyl]pyrazin-2-yl]amino]propyl]-2,2-dimethyl-pyrrolidine-1-carboxylate was isolated as a tan solid (220 mg, 42%). ESI-MS m/z calc. 592.2843, found 593.4 (M+1)⁺; Retention time: 1.59 minutes (LC method G).

### Step 4: 6-tert-Butyl-N-[6-[3-(5,5-dimethylpyrrolidin-3-yl)propylamino]pyrazin-2-yl]sulfonyl-2-fluoro-pyridine-3-carboxamide

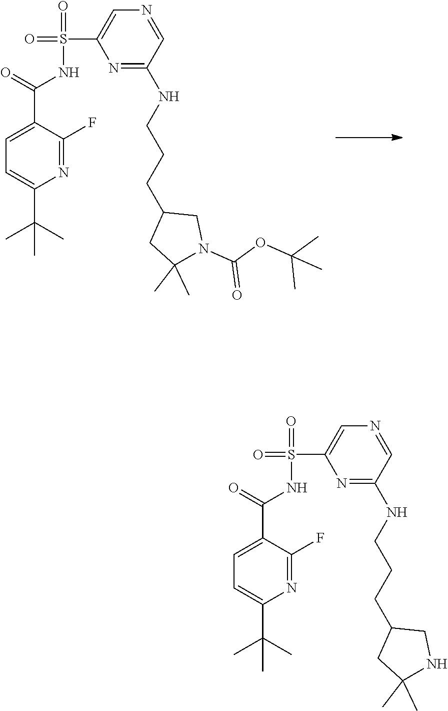 [SMILES: CC(C)(C)OC(=O)N1CC(CCCNc2cncc(S(=O)(=O)NC(=O)c3ccc(C(C)(C)C)nc3F)n2)CC1(C)C ; CC1(C)CC(CCCNc2cncc(S(=O)(=O)NC(=O)c3ccc(C(C)(C)C)nc3F)n2)CN1]

To a round bottom flask containing tert-butyl 4-[3-[[6-[(6-tert-butyl-2-fluoro-pyridine-3-carbonyl)sulfamoyl]pyrazin-2-yl]amino]propyl]-2,2-dimethyl-pyrrolidine-1-carboxylate (220 mg, 0.3712 mmol) was added DCM (3 mL) and HCl (3 mL of 4 M, 12.00 mmol). After 1 h, the reaction was evaporated to dryness and placed on high vacuum for 3 h to provide 6-tert-butyl-N-[6-[3-(5,5-dimethylpyrrolidin-3-yl)propylamino]pyrazin-2-yl]sulfonyl-2-fluoro-pyridine-3-carboxamide (hydrochloride salt) (196 mg, 100%). ESI-MS m/z calc. 492.2319, found 493.4 (M+1)⁺; Retention time: 0.37 minutes (LC method G).

### Step 5: 8-tert-Butyl-12,12-dimethyl-2λ⁶-thia-3,9,11,18,21,23-hexaazatetracyclo[17.3.1.111,14.05,10]tetracosa-1(22),5,7,9,19(23),20-hexaene-2,2,4-triune

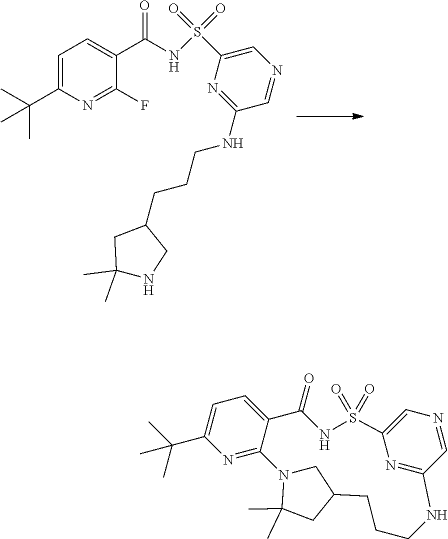 [SMILES: CC1(C)CC(CCCNc2cncc(S(=O)(=O)NC(=O)c3ccc(C(C)(C)C)nc3F)n2)CN1 ; CC(C)(C)c1ccc2c(n1)N1CC(CCCNc3cncc(n3)S(=O)(=O)NC2=O)CC1(C)C]

To a microwave vial was added potassium carbonate (512 mg, 3.705 mmol), 3 Å molecular sieves (10 pieces), and a solution of 6-tert-butyl-N-[6-[3-(5,5-dimethylpyrrolidin-3-yl)propylamino]pyrazin-2-yl]sulfonyl-2-fluoro-pyridine-3-carboxamide (hydrochloride salt) (196 mg, 0.3705 mmol) in NMP (12 mL). The reaction was heated at 150° C. in a preheated oil bath overnight. The reaction was cooled to rt, quenched with 1N HCl and extracted with EtOAc. The organic layer was dried over sodium sulfate, evaporated, and purified via reverse phase chromatography using 20%-99% ACN:H₂O with an HCl modifier. 8-tert-butyl-12,12-dimethyl-2λ⁶-thia-3,9,11,18,21,23-hexaazatetracyclo[17.3.1.111,14.05,10]tetracosa-1(22),5,7,9,19(23),20-hexaene-2,2,4-trione was isolated as a yellow solid (91 mg, 52%). ESI-MS m/z calc. 472.22565, found 473.4 (M+1)⁺; Retention time: 1.27 minutes (LC method G).

### Step 6: 8-tert-Butyl-12,12-dimethyl-2λ⁶-thia-3,9,11,18,21,23-hexaazatetracyclo[17.3.1.111,14.05,10]tetracosa-1(22),5,7,9,19(23),20-hexaene-2,2,4-trione, Compound 253 (enantiomer 1), and 8-tert-butyl-12,12-dimethyl-2λ⁶-thia-3,9,11,18,21,23-hexaazatetracyclo[17.3.1.111,14.05,10]tetracosa-1(22),5,7,9,19(23),20-hexaene-2,2,4-trione, Compound 252 (enantiomer 2)

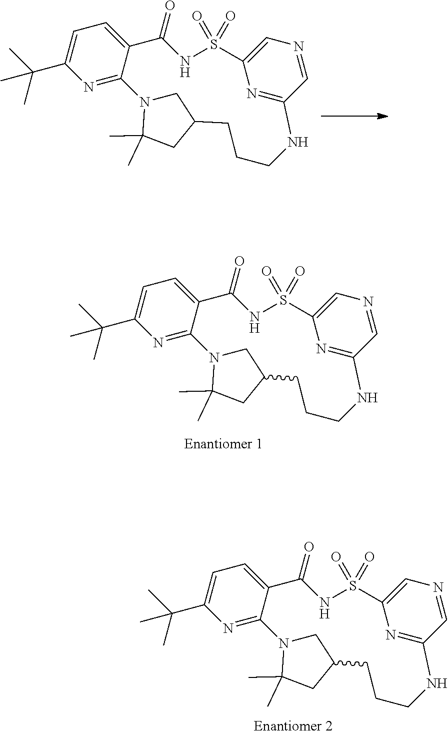 [SMILES: CC(C)(C)c1ccc2c(n1)N1CC(CCCNc3cncc(n3)S(=O)(=O)NC2=O)CC1(C)C ; CC(C)(C)c1ccc2c(n1)N1CC(CCCNc3cncc(n3)S(=O)(=O)NC2=O)CC1(C)C ; CC(C)(C)c1ccc2c(n1)N1CC(CCCNc3cncc(n3)S(=O)(=O)NC2=O)CC1(C)C]

8-tert-butyl-12,12-dimethyl-2λ⁶-thia-3,9,11,18,21,23-hexaazatetracyclo[17.3.1.111,14.05,10]tetracosa-1 (22),5,7,9,19(23),20-hexaene-2,2,4-trione (91 mg, 0.1926 mmol) was subjected to chiral SFC using the following method: ChiralPaK IG (250×21.2 mm, 5 μm) column, 40° C., mobile phase 22% MeOH, 78% CO₂, flow 70 mL/min, concentration 30 mg/mL in MeOH:DMSO (83:17), injection volume 500 μL, pressure 142 bar, wavelength 218 nm. Two isomers were isolated:

Enantiomer 1, peak 1: 8-tert-Butyl-12,12-dimethyl-2λ⁶-thia-3,9,11,18,21,23-hexaazatetracyclo[17.3.1.111,14.05,10]tetracosa-1(22),5,7,9,19(23),20-hexaene-2,2,4-trione (31.3 mg, 69%). ESI-MS m/z calc. 472.22565, found 473.1 (M+1)⁺; Retention time: 1.91 minutes (LC method A).

Enantiomer 2, peak 2: 8-tert-Butyl-12,12-dimethyl-2λ⁶-thia-3,9,11,18,21,23-hexaazatetracyclo[17.3.1.111,14.05,10]tetracosa-1(22),5,7,9,19(23),20-hexaene-2,2,4-trione (34.5 mg, 76%). ESI-MS m/z calc. 472.22565, found 473.1 (M+1)⁺; Retention time: 1.91 minutes (LC method A).

### Example 74: Preparation of 8-tert-butyl-21-hydroxy-12,12-dimethyl-2λ⁶-thia-3,9,11,18,23-pentaazatetracyclo[17.3.1.111,14.05,10]tetracosa-1(23),5,7,9,19,21-hexaene-2,2,4-trione, Compound 251 (enantiomer 1) and Compound 250 (enantiomer 2)

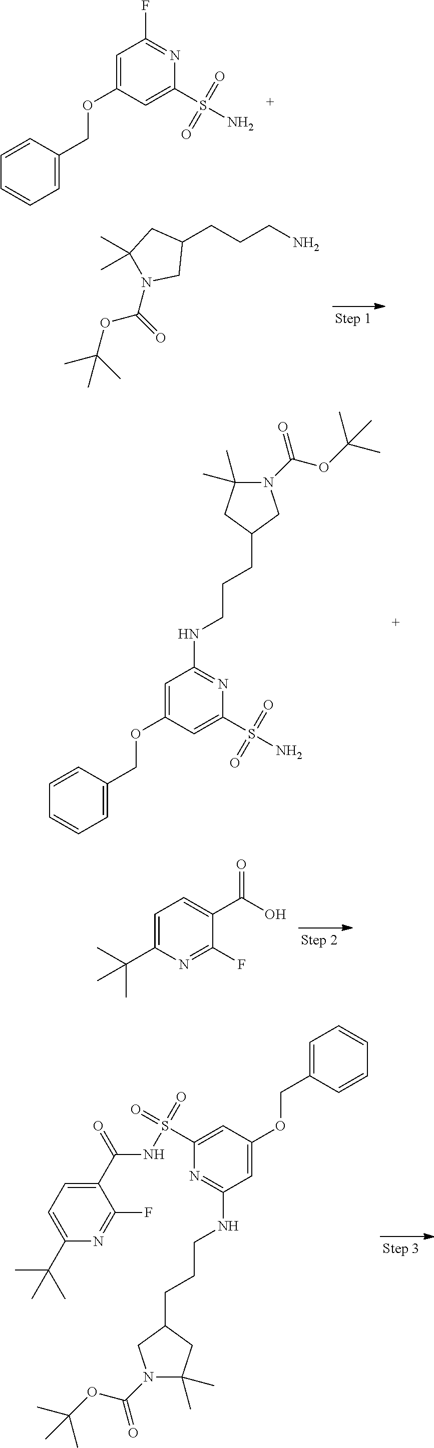

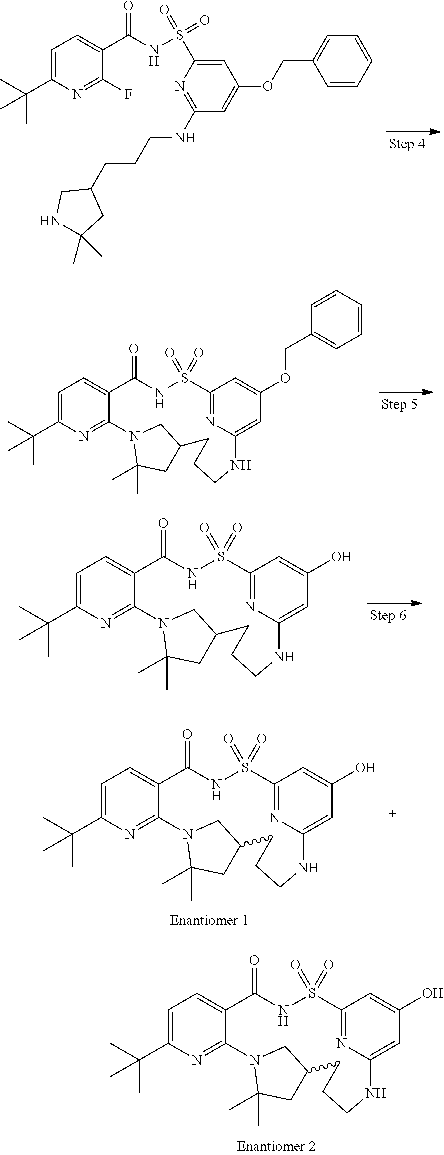

### Step 1: tert-Butyl 4-[3-[(4-benzyloxy-6-sulfamoyl-2-pyridyl)amino]propyl]-2,2-dimethyl-pyrrolidine-1-carboxylate

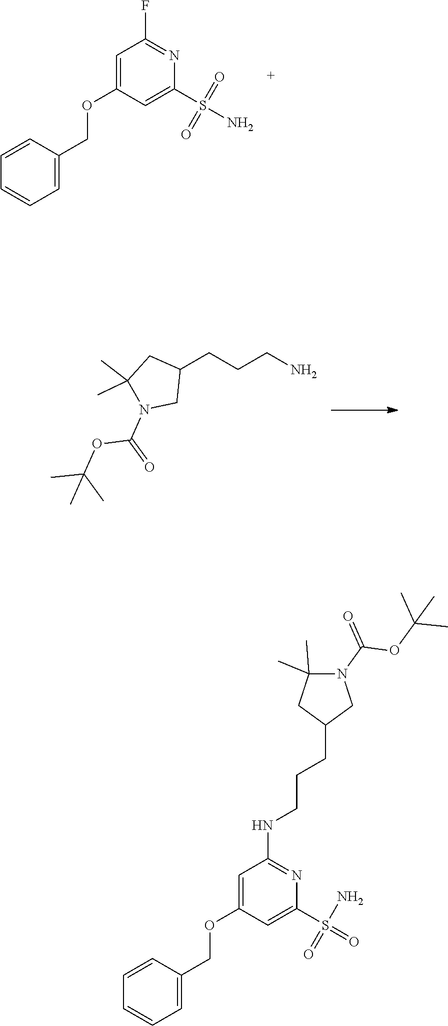 [SMILES: NS(=O)(=O)c1cc(OCc2ccccc2)cc(F)n1 ; CC(C)(C)OC(=O)N1CC(CCCN)CC1(C)C ; CC(C)(C)OC(=O)N1CC(CCCNc2cc(OCc3ccccc3)cc(S(N)(=O)=O)n2)CC1(C)C]

In a 20 mL microwave vial was added 4-benzyloxy-6-fluoro-pyridine-2-sulfonamide (500 mg, 1.771 mmol), tert-butyl 4-(3-aminopropyl)-2,2-dimethyl-pyrrolidine-1-carboxylate (324 mg, 1.264 mmol), Diisopropylethyl amine (1.1 mL, 6.315 mmol), and 1-butanol (3.75 mL). The vial was sealed and stirred at 140° C. overnight. UPLC showed conversion to the desired product. The reaction was cooled to rt and poured into water (100 mL) and extracted with ethyl acetate (2×50 mL). Combined organic layers, washed with water (2×50 mL), dried over sodium sulfate, filtered and concentrated. The orange residue was purified by silica gel chromatography (40 gram column, ELSD detection) using a gradient from 100% hexanes to 80% ethyl acetate in hexanes to afford a white foam tert-butyl 4-[3-[(4-benzyloxy-6-sulfamoyl-2-pyridyl)amino]propyl]-2,2-dimethyl-pyrrolidine-1-carboxylate (453 mg, 69%). ESI-MS m/z calc. 518.2563, found 519.4 (M+1)⁺; Retention time: 1.96 minutes (LC method A).

### Step 2: tert-Butyl 4-[3-[[4-benzyloxy-6-[(6-tert-butyl-2-fluoro-pyridine-3-carbonyl)sulfamoyl]-2-pyridyl]amino]propyl]-2,2-dimethyl-pyrrolidine-1-carboxylate

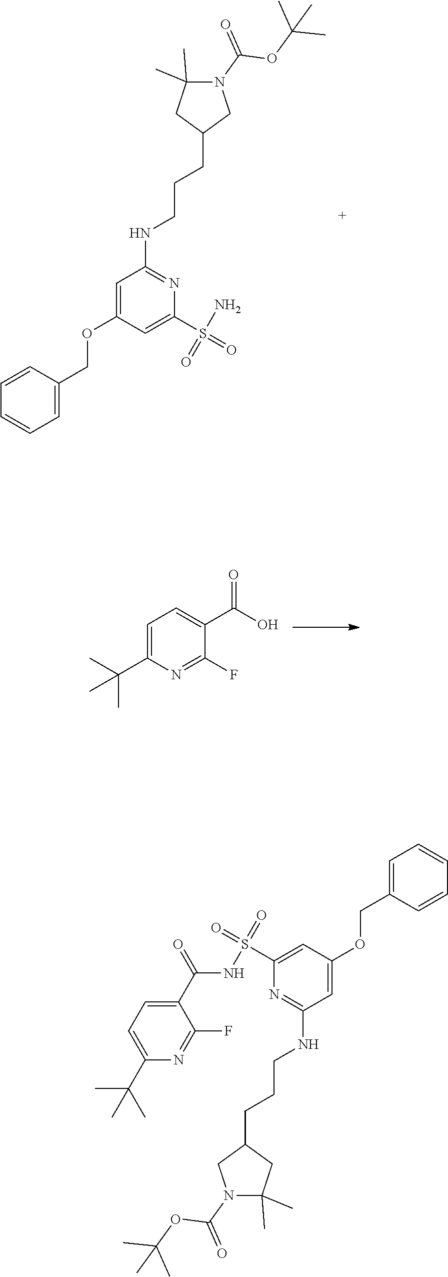

To a round bottom flask was added 6-tert-butyl-2-fluoro-pyridine-3-carboxylic acid (223.8 mg, 1.135 mmol), carbonyl diimidazole (184 mg, 1.135 mmol), and THF (4.5 mL). The reaction was purged with nitrogen and allowed to stir at 35° C. for 4 h. At this point, a solution of tert-butyl 4-[3-[(4-benzyloxy-6-sulfamoyl-2-pyridyl)amino]propyl]-2,2-dimethyl-pyrrolidine-1-carboxylate (453 mg, 0.8734 mmol) in THF (4.5 mL) was added followed by DBU (400 μL, 2.675 mmol). The reaction was allowed to stir at rt over the weekend. By UPLC analysis, the reaction showed the desired product. The reaction was quenched with 1N citric acid and extracted with EtOAc three times. The organic layers were dried over sodium sulfate, and evaporated. The crude oil was dissolved in DMF and purified via reverse phase chromatography 70%-99% ACN:H₂O with an HCl modifier. tert-butyl 4-[3-[[4-benzyloxy-6-[(6-tert-butyl-2-fluoro-pyridine-3-carbonyl)sulfamoyl]-2-pyridyl]amino]propyl]-2,2-dimethyl-pyrrolidine-1-carboxylate was isolated as a white solid (170 mg, 28%). ESI-MS m/z calc. 697.33093, found 698.5 (M+1)⁺; Retention time: 1.92 minutes (LC method G).

### Step 3: N-[[4-Benzyloxy-6-[3-(5,5-dimethylpyrrolidin-3-yl)propylamino]-2-pyridyl]sulfonyl]-6-tert-butyl-2-fluoro-pyridine-3-carboxamide

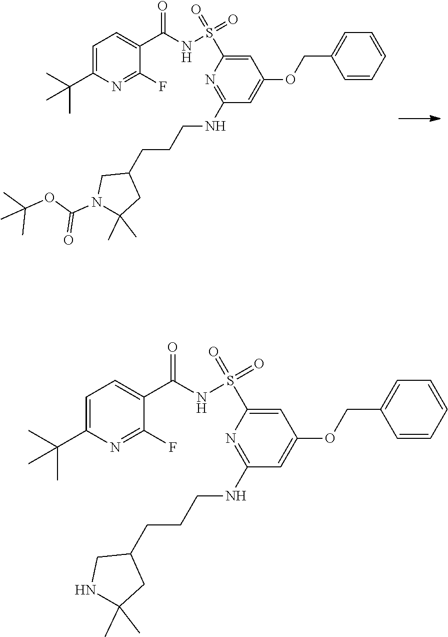 [SMILES: CC(C)(C)OC(=O)N1CC(CCCNc2cc(OCc3ccccc3)cc(S(=O)(=O)NC(=O)c3ccc(C(C)(C)C)nc3F)n2)CC1(C)C ; CC1(C)CC(CCCNc2cc(OCc3ccccc3)cc(S(=O)(=O)NC(=O)c3ccc(C(C)(C)C)nc3F)n2)CN1]

To a round bottom flask containing tert-butyl 4-[3-[[4-benzyloxy-6-[(6-tert-butyl-2-fluoro-pyridine-3-carbonyl)sulfamoyl]-2-pyridyl]amino]propyl]-2,2-dimethyl-pyrrolidine-1-carboxylate (170 mg, 0.2436 mmol) was added DCM (3 mL) and HCl (3 mL of 4 M in dioxane, 12.00 mmol). After 1 h at rt, the reaction was evaporated and dried under high vacuum for 3 h to provide N-[[4-benzyloxy-6-[3-(5,5-dimethylpyrrolidin-3-yl)propylamino]-2-pyridyl]sulfonyl]-6-tert-butyl-2-fluoro-pyridine-3-carboxamide (hydrochloride salt) (154 mg, 100%). ESI-MS m/z calc. 597.2785, found 598.3 (M+1)⁺; Retention time: 0.77 minutes (LC method G).

### Step 4: 21-(Benzyloxy)-8-tert-butyl-12,12-dimethyl-2λ⁶-thia-3,9,11,18,23-pentaazatetracyclo[17.3.1.111,14.05,10]tetracosa-1(23),5,7,9,19,21-hexaene-2,2,4-trione

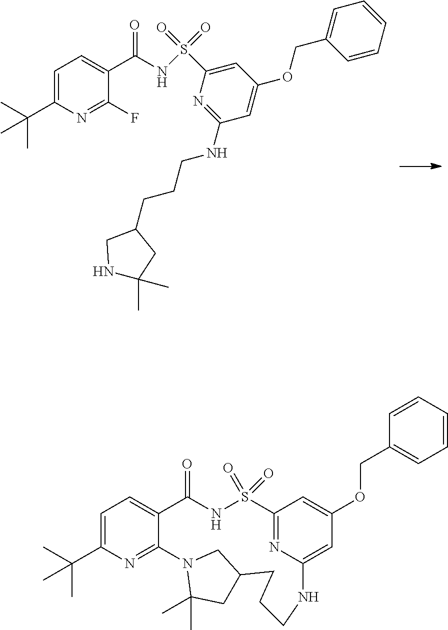 [SMILES: CC1(C)CC(CCCNc2cc(OCc3ccccc3)cc(S(=O)(=O)NC(=O)c3ccc(C(C)(C)C)nc3F)n2)CN1 ; CC(C)(C)c1ccc2c(n1)N1CC(CCCNc3cc(OCc4ccccc4)cc(n3)S(=O)(=O)NC2=O)CC1(C)C]

To a microwave vial was added potassium carbonate (336 mg, 2.431 mmol), 3 Å molecular sieves, and a solution of N-[[4-benzyloxy-6-[3-(5,5-dimethylpyrrolidin-3-yl)propylamino]-2-pyridyl]sulfonyl]-6-tert-butyl-2-fluoro-pyridine-3-carboxamide (hydrochloride salt) (154 mg, 0.2428 mmol) in NMP (9.2 mL). The reaction was heated at 150° C. in a preheated oil bath overnight. The reaction was cooled to rt, quenched with 1N HCl and extracted with EtOAc. The organic layer was dried over sodium sulfate, evaporated, and purified via reverse phase chromatography using 40%-99% ACN:H₂O with an HCl modifier. 21-(Benzyloxy)-8-tert-butyl-12,12-dimethyl-2λ⁶-thia-3,9,11,18,23-pentaazatetracyclo[17.3.1.111,14.05,10]tetracosa-1(23),5,7,9,19,21-hexaene-2,2,4-trione was isolated as a white solid (88 mg, 63%). ESI-MS m/z calc. 577.2723, found 578.4 (M+1)⁺; Retention time: 1.76 minutes (LC method G).

### Step 5: 8-tert-Butyl-21-hydroxy-12,12-dimethyl-2λ⁶-thia-3,9,11,18,23-pentaazatetracyclo[17.3.1.111,14.05,10]tetracosa-1(23),5,7,9,19,21-hexaene-2,2,4-trione

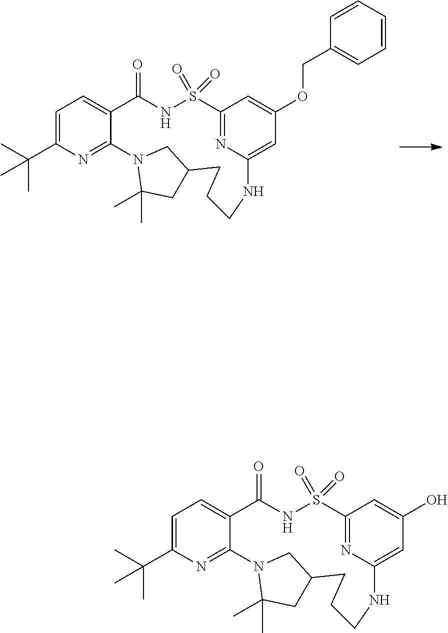 [SMILES: CC(C)(C)c1ccc2c(n1)N1CC(CCCNc3cc(OCc4ccccc4)cc(n3)S(=O)(=O)NC2=O)CC1(C)C ; CC(C)(C)c1ccc2c(n1)N1CC(CCCNc3cc(O)cc(n3)S(=O)(=O)NC2=O)CC1(C)C]

To a round bottom flask containing 21-(benzyloxy)-8-tert-butyl-12,12-dimethyl-2λ⁶-thia-3,9,11,18,23-pentaazatetracyclo[17.3.1.111,14.05,10]tetracosa-1(23),5,7,9,19,21-hexaene-2,2,4-trione (88 mg, 0.1523 mmol) was added Pd on C, wet, Degussa (88 mg of 10% w/w, 0.08269 mmol) and isopropanol (3 mL). The reaction was purged with nitrogen for 5 minutes and then a balloon of hydrogen gas was added to the reaction overnight. The reaction was filtered and purified via reverse phase chromatography (40%-99% ACN:H₂O with an HCl modifier) to provide 8-tert-butyl-21-hydroxy-12,12-dimethyl-2λ⁶-thia-3,9,11,18,23-pentaazatetracyclo[17.3.1.111,14.05,10]tetracosa-1 (23),5,7,9,19,21-hexaene-2,2,4-trione (53 mg, 71%). ESI-MS m/z calc. 487.2253, found 488.4 (M+1)⁺; Retention time: 1.17 minutes (LC method G).

### Step 6: 8-tert-Butyl-21-hydroxy-12,12-dimethyl-2λ⁶-thia-3,9,11,18,23-pentaazatetracyclo[17.3.1.111,14.05,10]tetracosa-1(23),5,7,9,19,21-hexaene-2,2,4-trione, Compound 251 (enantiomer 1), and 8-tert-butyl-21-hydroxy-12,12-dimethyl-2λ⁶-thia-3,9,11,18,23-pentaazatetracyclo[17.3.1.111,14.05,10]tetracosa-1(23),5,7,9,19,21-hexaene-2,2,4-trione, Compound 250 (enantiomer 2)

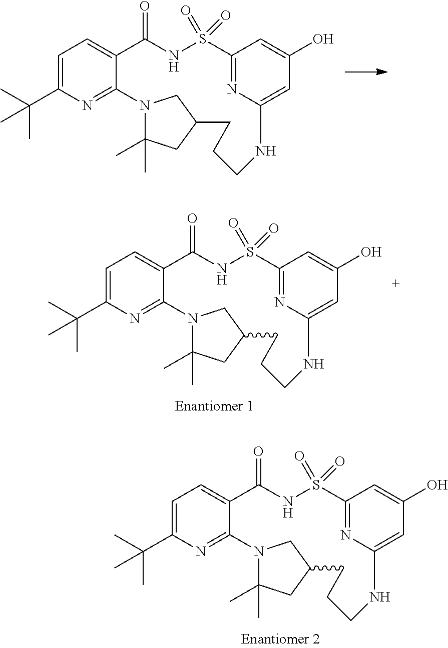 [SMILES: CC(C)(C)c1ccc2c(n1)N1CC(CCCNc3cc(O)cc(n3)S(=O)(=O)NC2=O)CC1(C)C ; CC(C)(C)c1ccc2c(n1)N1CC(CCCNc3cc(O)cc(n3)S(=O)(=O)NC2=O)CC1(C)C ; CC(C)(C)c1ccc2c(n1)N1CC(CCCNc3cc(O)cc(n3)S(=O)(=O)NC2=O)CC1(C)C]

8-tert-Butyl-21-hydroxy-12,12-dimethyl-2λ⁶-thia-3,9,11,18,23-pentaazatetracyclo[17.3.1.111,14.05,10]tetracosa-1(23),5,7,9,19,21-hexaene-2,2,4-trione (53 mg, 0.1087 mmol) was subjected to chiral SFC using the following method: ChiralPaK IG (250×10 mm, 5 μm) column, 35° C., mobile phase 30% MeOH, 70% CO₂, flow 10 mL/min, concentrations 23 mg/mL in MeOH, injection volume 70 μL, pressure 174 bar, wavelength 223 nm, to give two enantiomers:

First of elute, enantiomer 1: 8-tert-Butyl-21-hydroxy-12,12-dimethyl-2λ⁶-thia-3,9,11,18,23-pentaazatetracyclo[17.3.1.111,14.05,10]tetracosa-1(23),5,7,9,19,21-hexaene-2,2,4-trione (18.0 mg, 68%). ESI-MS m/z calc. 487.2253, found 488.1 (M+1)⁺; Retention time: 1.83 minutes (LC method A).

Second to elute, enantiomer 2: 8-tert-Butyl-21-hydroxy-12,12-dimethyl-2λ⁶-thia-3,9,11,18,23-pentaazatetracyclo[17.3.1.111,14.05,10]tetracosa-1 (23),5,7,9,19,21-hexaene-2,2,4-trione (13.2 mg, 49%) ESI-MS m/z calc. 487.2253, found 488.1 (M+1)⁺; Retention time: 1.83 minutes (LC method A).

### Example 75: Preparation of 8-tert-butyl-12,12-dimethyl-18-oxa-2λ⁶-thia-3,9,11-triazatetracyclo[17.3.1.111,14.05,10]tetracosa-1(22),5,7,9,19(23),20-hexaene-2,2,4-trione, Compound 257 (enantiomer 1) and Compound 256 (enantiomer 2)

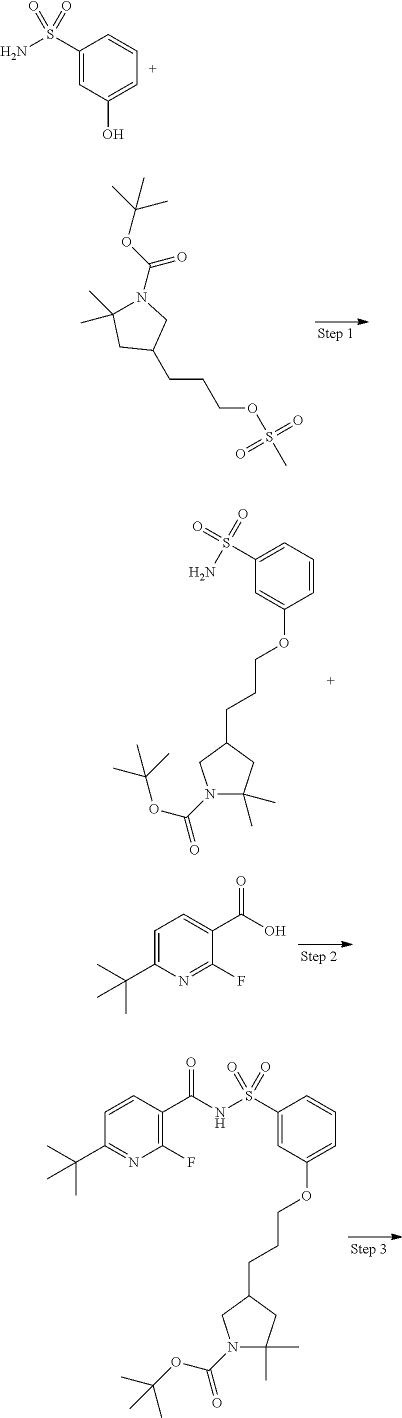 [SMILES: NS(=O)(=O)c1cccc(O)c1 ; CC(C)(C)OC(=O)N1CC(CCCOS(C)(=O)=O)CC1(C)C ; CC(C)(C)OC(=O)N1CC(CCCOc2cccc(S(N)(=O)=O)c2)CC1(C)C ; CC(C)(C)c1ccc(C(=O)O)c(F)n1 ; CC(C)(C)OC(=O)N1CC(CCCOc2cccc(S(=O)(=O)NC(=O)c3ccc(C(C)(C)C)nc3F)c2)CC1(C)C]

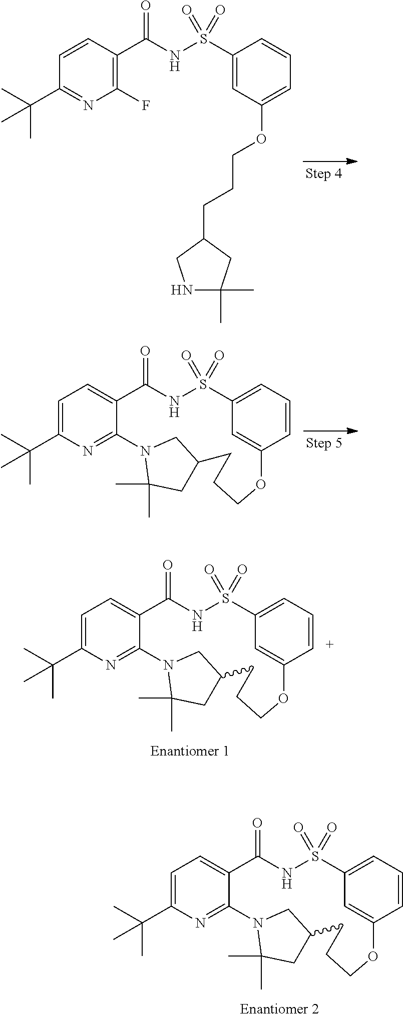

### Step 1: tert-Butyl 2,2-dimethyl-4-[3-(3-sulfamoylphenoxy)propyl]pyrrolidine-1-carboxylate

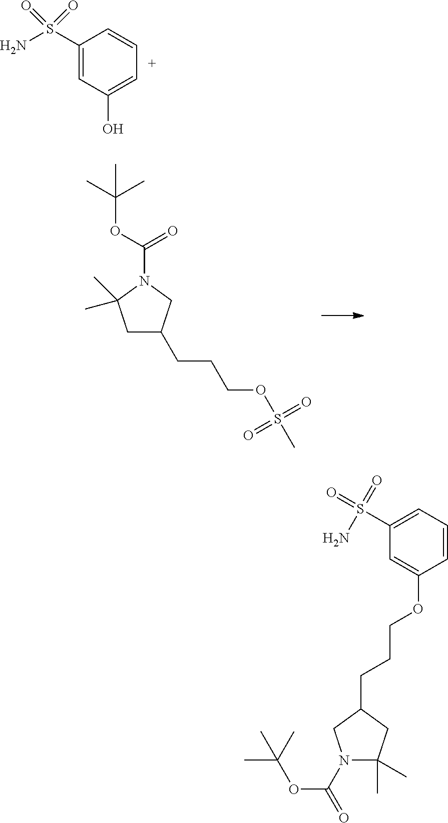

In a 100 mL flask was added 3-hydroxybenzenesulfonamide (350 mg, 2.021 mmol) and DMF (8.0 mL). To the mixture was added K₂CO₃ (850 mg, 6.150 mmol) and {tert}-butyl 2,2-dimethyl-4-(3-methylsulfonyloxypropyl)pyrrolidine-1-carboxylate (675 mg, 2.012 mmol) at room temperature. The resulting mixture was heated to 50° C. for 18 h. The mixture was then poured into IN citric acid and extracted with EtOAc (2×). Combined organic fractions, dried (sodium sulfate), filtered and concentrated to a yellow oil which was purified on silica gel chromatography (80 gram column) using a gradient from 100% hexanes to 65% ethyl acetate in hexanes to afford as a light yellow foam tert-butyl 2,2-dimethyl-4-[3-(3-sulfamoylphenoxy)propyl]pyrrolidine-1-carboxylate (341.3 mg, 41%). ¹H NMR (499 MHz, DMSO-d₆) δ 7.46 (t, J=8.0 Hz, 1H), 7.40-7.36 (m, 1H), 7.33 (d, J=10.1 Hz, 3H), 7.18-7.09 (m, 1H), 4.02 (t, J=6.5 Hz, 2H), 3.58 (q, J=8.6 Hz, 1H), 2.81 (q, J=11.0 Hz, 1H), 2.16 (d, J=12.0 Hz, 1H), 1.92 (ddd, J=18.4, 12.1, 6.0 Hz, 1H), 1.74 (dq, J=15.9, 6.9 Hz, 2H), 1.46 (q, J=9.7, 8.3 Hz, 3H), 1.38 (t, J=12.9 Hz, 12H), 1.25 (s, 3H). ESI-MS m/z calc. 412.2032, found 413.2 (M+1)⁺; Retention time: 1.79 minutes (LC method A).

### Step 2: tert-Butyl 4-[3-[3-[(6-tert-butyl-2-fluoro-pyridine-3-carbonyl)sulfamoyl]phenoxy]propyl]-2,2-dimethyl-pyrrolidine-1-carboxylate

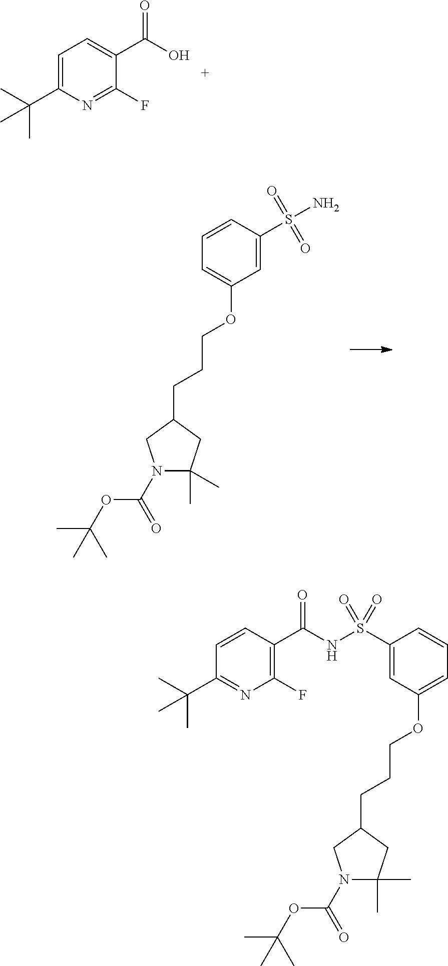 [SMILES: CC(C)(C)c1ccc(C(=O)O)c(F)n1 ; CC(C)(C)OC(=O)N1CC(CCCOc2cccc(S(N)(=O)=O)c2)CC1(C)C ; CC(C)(C)OC(=O)N1CC(CCCOc2cccc(S(=O)(=O)NC(=O)c3ccc(C(C)(C)C)nc3F)c2)CC1(C)C]

To a solution of 6-tert-butyl-2-fluoro-pyridine-3-carboxylic acid (296 mg, 1.501 mmol) in THF (7 mL) was added CDI (255 mg, 1.573 mmol) and the mixture was stirred at rt for 20 h. Then tert-butyl 2,2-dimethyl-4-[3-(3-sulfamoylphenoxy)propyl]pyrrolidine-1-carboxylate (341 mg, 0.8266 mmol) was added followed by DBU (425 μL, 2.842 mmol) and the resulting mixture was stirred for 3 h at rt. The reaction was diluted with ethyl acetate and washed with a saturated aqueous sodium bicarbonate solution. The organic layer was further washed with 10% citric acid solution followed by brine. The organics were separated, dried over sodium sulfate, evaporated and then purified on silica gel chromatography (80 gram column) using a gradient from 100% hexanes to 100% ethyl acetate to afford tert-butyl 4-[3-[3-[(6-tert-butyl-2-fluoro-pyridine-3-carbonyl)sulfamoyl]phenoxy]propyl]-2,2-dimethyl-pyrrolidine-1-carboxylate (475 mg, 83%) as an off-white solid. ESI-MS m/z calc. 591.27783, found 592.2 (M+1)⁺; Retention time: 2.31 minutes (LC method A).

### Step 3: 6-tert-Butyl-N-[3-[3-(5,5-dimethylpyrrolidin-3-yl)propoxy]phenyl]sulfonyl-2-fluoro-pyridine-3-carboxamide

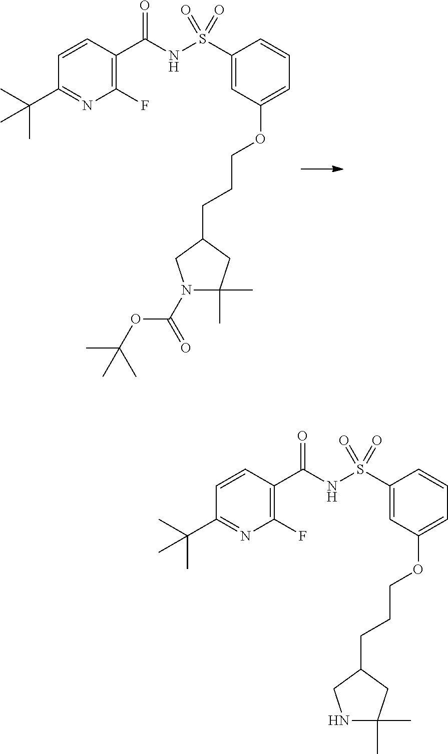 [SMILES: CC(C)(C)OC(=O)N1CC(CCCOc2cccc(S(=O)(=O)NC(=O)c3ccc(C(C)(C)C)nc3F)c2)CC1(C)C ; CC1(C)CC(CCCOc2cccc(S(=O)(=O)NC(=O)c3ccc(C(C)(C)C)nc3F)c2)CN1]

tert-Butyl 4-[3-[3-[(6-tert-butyl-2-fluoro-pyridine-3-carbonyl)sulfamoyl]phenoxy]propyl]-2,2-dimethyl-pyrrolidine-1-carboxylate (475 mg, 0.6823 mmol) was dissolved in DCM (9 mL) and to the mixture was added TFA (1.5 mL, 19.47 mmol) and stirred at room temperature. After 14 h, the mixture was evaporated to dryness, then diluted with diethyl ether (30 mL×2), and reconcentrated. The material was then placed on the high vacuum pump for 2 h to afford 6-tert-butyl-N-[3-[3-(5,5-dimethylpyrrolidin-3-yl)propoxy]phenyl]sulfonyl-2-fluoro-pyridine-3-carboxamide (Trifluoroacetate salt) as a pale yellow oil (450 mg, 98%). ESI-MS m/z calc. 491.2254, found 492.2 (M+1)⁺; Retention time: 1.39 minutes (LC method A).

### Step 4: 8-tert-Butyl-12,12-dimethyl-18-oxa-2λ⁶-thia-3,9,11-triazatetracyclo [17.3.1.111,14.05,10]tetracosa-1(22),5,7,9,19(23),20-hexaene-2,2,4-trione

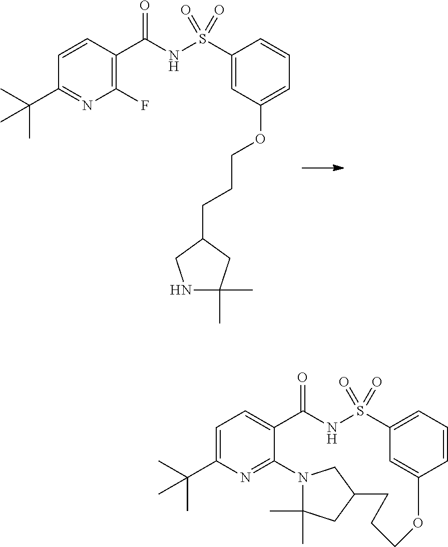 [SMILES: CC1(C)CC(CCCOc2cccc(S(=O)(=O)NC(=O)c3ccc(C(C)(C)C)nc3F)c2)CN1 ; CC(C)(C)c1ccc2c(n1)N1CC(CCCOc3cccc(c3)S(=O)(=O)NC2=O)CC1(C)C]

To a solution of 6-tert-butyl-N-[3-[3-(5,5-dimethylpyrrolidin-3-yl)propoxy]phenyl]sulfonyl-2-fluoro-pyridine-3-carboxamide (Trifluoroacetate salt) (450 mg, 0.6687 mmol) in NMP (24.36 mL) was added potassium carbonate (648.7 mg, 4.694 mmol). The mixture was purged with nitrogen for 5 min. The mixture was heated at 150° C. and stirred overnight. Cooled to room temperature, diluted with EtOAc then washed with 1N HCl, dried (sodium sulfate), filtered and concentrated to a brown oil which was filtered and purified by reverse phase HPLC-MS method using a Luna C₁₈ column (75×30 mm, 5 μm particle size) sold by Phenomenex (pn: 00C-4252-U0-AX), and a dual gradient run from 1-99% mobile phase B over 15.0 minutes (mobile phase A=H₂O (5 mM HCl), mobile phase B=acetonitrile, flow rate=50 mL/min, injection volume=950 μL and column temperature=25° C.) giving as a white solid, 8-tert-Butyl-12,12-dimethyl-18-oxa-2λ⁶-thia-3,9,11-triazatetracyclo[17.3.1.111,14.05,10]tetracosa-1(22),5,7,9,19(23),20-hexaene-2,2,4-trione (129.6 mg, 41%). ESI-MS m/z calc. 471.21918, found 472.3 (M+1)⁺; Retention time: 2.08 minutes (LC method A).

### Step 5: 8-tert-Butyl-12,12-dimethyl-18-oxa-2λ⁶-thia-3,9,11-triazatetracyclo [17.3.1.111,14.05,10]tetracosa-1(22),5,7,9,19(23),20-hexaene-2,2,4-trione, Compound 257 (enantiomer 1), and 8-tert-butyl-12,12-dimethyl-18-oxa-2λ⁶-thia-3,9,11-triazatetracyclo[17.3.1.111,14.05,10]tetracosa-1(22),5,7,9,19(23),20-hexaene-2,2,4-trione, Compound 256 (enantiomer 2)

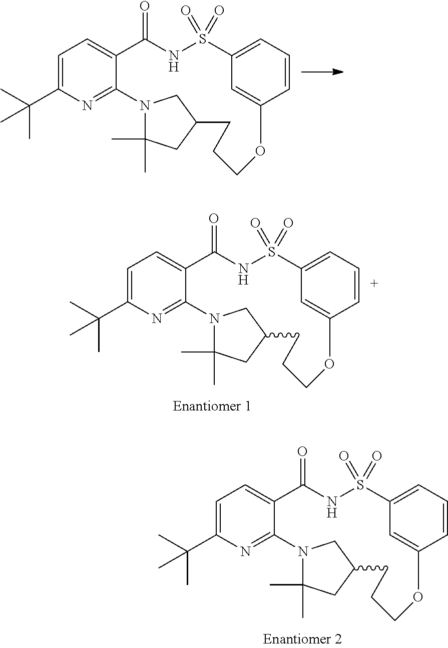 [SMILES: CC(C)(C)c1ccc2c(n1)N1CC(CCCOc3cccc(c3)S(=O)(=O)NC2=O)CC1(C)C ; CC(C)(C)c1ccc2c(n1)N1CC(CCCOc3cccc(c3)S(=O)(=O)NC2=O)CC1(C)C ; CC(C)(C)c1ccc2c(n1)N1CC(CCCOc3cccc(c3)S(=O)(=O)NC2=O)CC1(C)C]

Subjected 8-tert-butyl-12,12-dimethyl-18-oxa-2λ⁶-thia-3,9,11-triazatetracyclo[17.3.1.111,14.05,10]tetracosa-1(22),5,7,9,19(23),20-hexaene-2,2,4-trione (129.6 mg, 0.2748 mmol) to chiral separation by SFC chromatography using a ChiralPak IG (250×21.2 mm column, 5 μm particle size) with 30% MeOH/70% CO₂ mobile phase at 70 mL/min over 8.0 minutes (injection volume=500 μL of 32 mg/mL solution in 90/10 MeOH/DMSO giving two isomers:

First to elute, enantiomer 1: 8-tert-Butyl-12,12-dimethyl-18-oxa-2λ⁶-thia-3,9,11-triazatetracyclo[17.3.1.111,14.05,10]tetracosa-1(22),5,7,9,19(23),20-hexaene-2,2,4-trione (57.2 mg, 88%). ESI-MS m/z calc. 471.21918, found 472.2 (M+1)⁺; Retention time: 2.17 minutes (LC method Q).

Second to elute, enantiomer 2: 8-tert-Butyl-12,12-dimethyl-18-oxa-2λ⁶-thia-3,9,11-triazatetracyclo[17.3.1.111,14.05,10]tetracosa-1(22),5,7,9,19(23),20-hexaene-2,2,4-trione (52 mg, 80%). ESI-MS m/z calc. 471.21918, found 472.2 (M+1)⁺; Retention time: 2.19 minutes, both as white solids (LC method Q).

### Example 76: Preparation of 8-tert-Butyl-12,12-dimethyl-15-oxa-2λ⁶-thia-3,9,11,18,23-pentaazatetracyclo[17.3.1.111,14.05,10]tetracosa-1(22),5(10),6,8,19(23),20-hexaene-2,2,4-trione, Compound 259 (enantiomer 1), and Compound 258 (enantiomer 2)

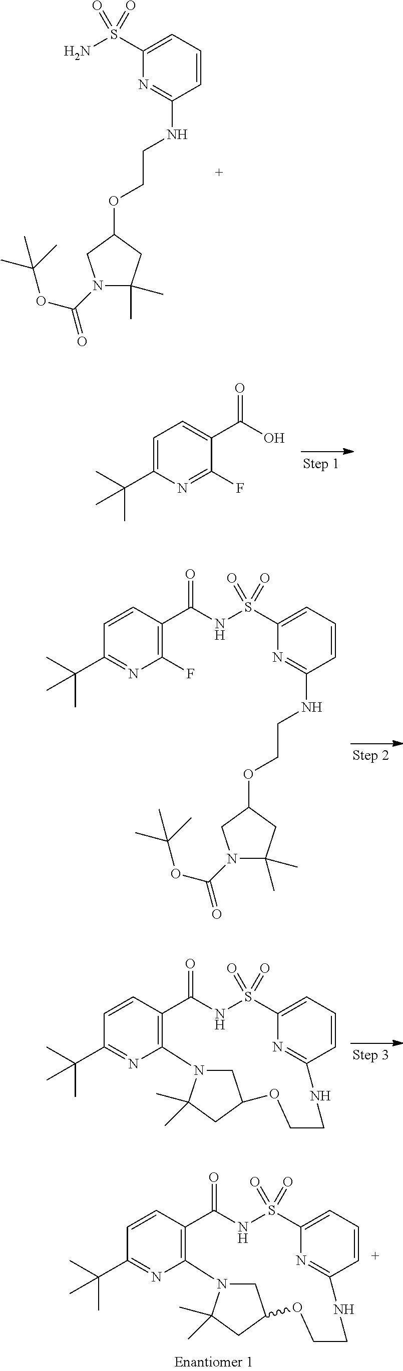 [SMILES: CC(C)(C)OC(=O)N1CC(OCCNc2cccc(S(N)(=O)=O)n2)CC1(C)C ; CC(C)(C)c1ccc(C(=O)O)c(F)n1 ; CC(C)(C)OC(=O)N1CC(OCCNc2cccc(S(=O)(=O)NC(=O)c3ccc(C(C)(C)C)nc3F)n2)CC1(C)C ; CC(C)(C)c1ccc2c(n1)N1CC(CC1(C)C)OCCNc1cccc(n1)S(=O)(=O)NC2=O ; CC(C)(C)c1ccc2c(n1)N1CC(CC1(C)C)OCCNc1cccc(n1)S(=O)(=O)NC2=O]

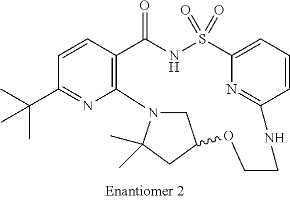 [SMILES: CC(C)(C)c1ccc2c(n1)N1CC(CC1(C)C)OCCNc1cccc(n1)S(=O)(=O)NC2=O]

### Step 1: tert-Butyl 4-[2-[[6-[(6-tert-butyl-2-fluoro-pyridine-3-carbonyl)sulfamoyl]-2-pyridyl]amino]ethoxy]-2,2-dimethyl-pyrrolidine-1-carboxylate

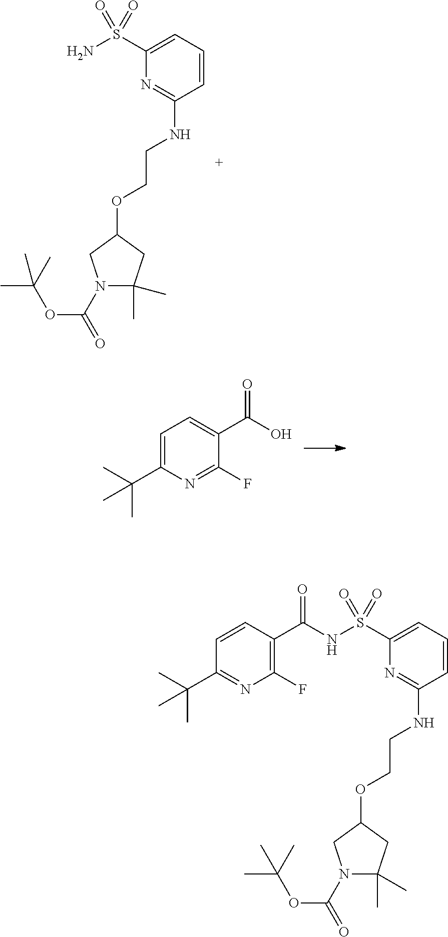 [SMILES: CC(C)(C)OC(=O)N1CC(OCCNc2cccc(S(N)(=O)=O)n2)CC1(C)C ; CC(C)(C)c1ccc(C(=O)O)c(F)n1 ; CC(C)(C)OC(=O)N1CC(OCCNc2cccc(S(=O)(=O)NC(=O)c3ccc(C(C)(C)C)nc3F)n2)CC1(C)C]

To a solution of the 6-tert-butyl-2-fluoro-pyridine-3-carboxylic acid (51 mg, 0.2586 mmol) in THF (2 mL) was added CDI (47 mg, 0.2899 mmol) and the mixture was stirred at rt for 1 h then tert-butyl 2,2-dimethyl-4-[2-[(6-sulfamoyl-2-pyridyl)amino]ethoxy]pyrrolidine-1-carboxylate (100 mg, 0.2412 mmol) was added followed by DBU (120 μL, 0.8024 mmol) and the resulting mixture was stirred for 16 h at room temperature. The reaction mixture was quenched with 1:1 mixture of saturated ammonium chloride solution and brine and extracted with ethyl acetate. Combined organic extracts washed with brine, dried over sodium sulphate, filtered and concentrated. The resultant brown residue was purified by silica gel column chromatography using a shallow gradient 100% hexanes to 100% EtOAc to afford tert-butyl 4-[2-[[6-[(6-tert-butyl-2-fluoro-pyridine-3-carbonyl)sulfamoyl]-2-pyridyl]amino]ethoxy]-2,2-dimethyl-pyrrolidine-1-carboxylate as an off-white solid (87 mg, 57%). ESI-MS m/z calc. 593.2683, found 594.54 (M+1)⁺; Retention time: 0.57 minutes (LC method G).

### Step 2: 8-tert-Butyl-12,12-dimethyl-15-oxa-2λ⁶-thia-3,9,11,18,23-pentaazatetracyclo [17.3.1.111,14.05,10]tetracosa-1(22),5(10),6,8,19(23),20-hexaene-2,2,4-trione

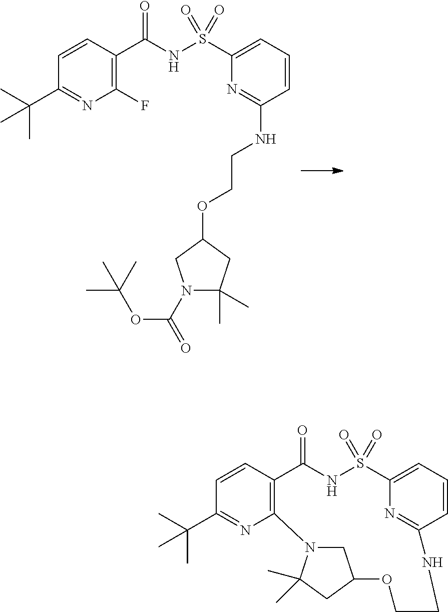 [SMILES: CC(C)(C)OC(=O)N1CC(OCCNc2cccc(S(=O)(=O)NC(=O)c3ccc(C(C)(C)C)nc3F)n2)CC1(C)C ; CC(C)(C)c1ccc2c(n1)N1CC(CC1(C)C)OCCNc1cccc(n1)S(=O)(=O)NC2=O]

A solution of tert-butyl 4-[2-[[6-[(6-tert-butyl-2-fluoro-pyridine-3-carbonyl)sulfamoyl]-2-pyridyl]amino]ethoxy]-2,2-dimethyl-pyrrolidine-1-carboxylate (87 mg, 0.1465 mmol) in premixed solution of 1:4 TFA (125 μL, 1.622 mmol) and DCM (500 μL) was stirred at rt for 1 h and after the deprotection was complete, the solvents were removed. The resultant residue was dissolved in NMP (2 mL) and potassium carbonate (174 mg, 1.259 mmol) was added. The mixture was purged with nitrogen for 5 min. The mixture was heated at 160° C. overnight. The mixture was cooled to room temperature, diluted with 1N HCl and extracted with EtOAc (1×). The organic phase was washed with brine (1×), dried (sodium sulfate), filtered and concentrated to a brown oil which was filtered and purified by reverse phase HPLC-MS method using a dual gradient run of 30-99% mobile phase B over 15.0 minutes (mobile phase A=H₂O (5 mM HCl), mobile phase B=acetonitrile) giving as an off-white solid, 8-tert-butyl-12,12-dimethyl-15-oxa-2λ⁶-thia-3,9,11,18,23-pentaazatetracyclo[17.3.1.111,14.05,10]tetracosa-1(22),5(10),6,8,19(23),20-hexaene-2,2,4-trione (42.5 mg, 61%). ESI-MS m/z calc. 473.2097, found 474.1 (M+1)⁺; Retention time: 0.98 minutes (LC method G).

### Step 3: 8-tert-Butyl-12,12-dimethyl-15-oxa-2λ⁶-thia-3,9,11,18,23-pentaazatetracyclo [17.3.1.111,14.05,10]tetracosa-1(22),5(10),6,8,19(23),20-hexaene-2,2,4-trione, Compound 259 (enantiomer 1), and 8-tert-butyl-12,12-dimethyl-15-oxa-2λ⁶-thia-3,9,11,18,23-pentaazatetracyclo[17.3.1.111,14.05,10]tetracosa-1(22),5(10),6,8,19(23),20-hexaene-2,2,4-trione, Compound 258 (enantiomer 2)

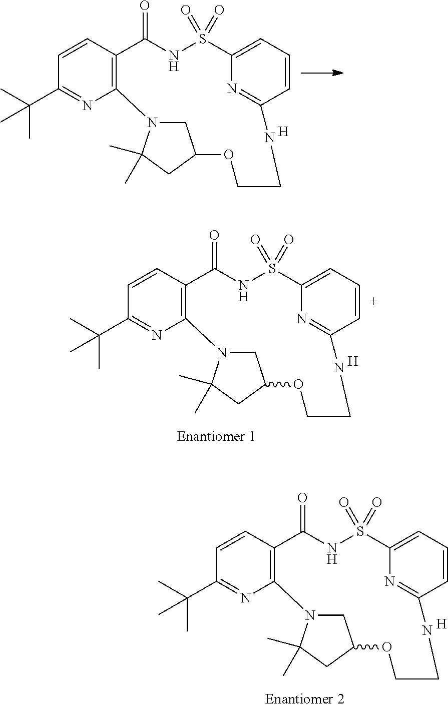

Racemic 8-tert-Butyl-12,12-dimethyl-15-oxa-2λ⁶-thia-3,9,11,18,23-pentaazatetracyclo[17.3.1.111,14.05,10]tetracosa-1(22),5(10),6,8,19(23),20-hexaene-2,2,4-trione (40 mg, 0.08446 mmol) was subjected to chiral separation by SFC chromatography using a Phenomenex LUX-4 (250×10 mm) 5 μm particle size column at 35° C., a 42% MeOH (no modifier), 58% CO₂ mobile phase, a 10 mL/min flow (concentration of 23 mg/mL in methanol, injection volume=70 μL, pressure 178 bar, wavelength 210 nm) giving two isomers:

First to elute, enantiomer 1: 8-tert-Butyl-12,12-dimethyl-15-oxa-2λ⁶-thia-3,9,11,18,23-pentaazatetracyclo[17.3.1.111,14.05,10]tetracosa-1(22),5(10),6,8,19(23),20-hexaene-2,2,4-trione (18 mg, 87%). ESI-MS m/z calc. 473.2097, found 474.1 (M+1)⁺; Retention time: 1.71 minutes (LC method G).

Second to elute, enantiomer 2: 8-tert-Butyl-12,12-dimethyl-15-oxa-2λ⁶-thia-3,9,11,18,23-pentaazatetracyclo[17.3.1.111,14.05,10]tetracosa-1(22),5(10),6,8,19(23),20-hexaene-2,2,4-trione (16 mg, 80%). ESI-MS m/z calc. 473.2097, found 474.3 (M+1)⁺; Retention time: 1.71 minutes (LC method G).

### Example 77: Preparation of (5S)-11-tert-Butyl-7,7-dimethyl-17λ⁶-thia-1,8,10,16-tetraazapentacyclo[16.5.2.15,8.09,14.021,24]hexacosa-9(14),10,12,18,20,22,24-heptaene-15,17,17-trione, Compound 260

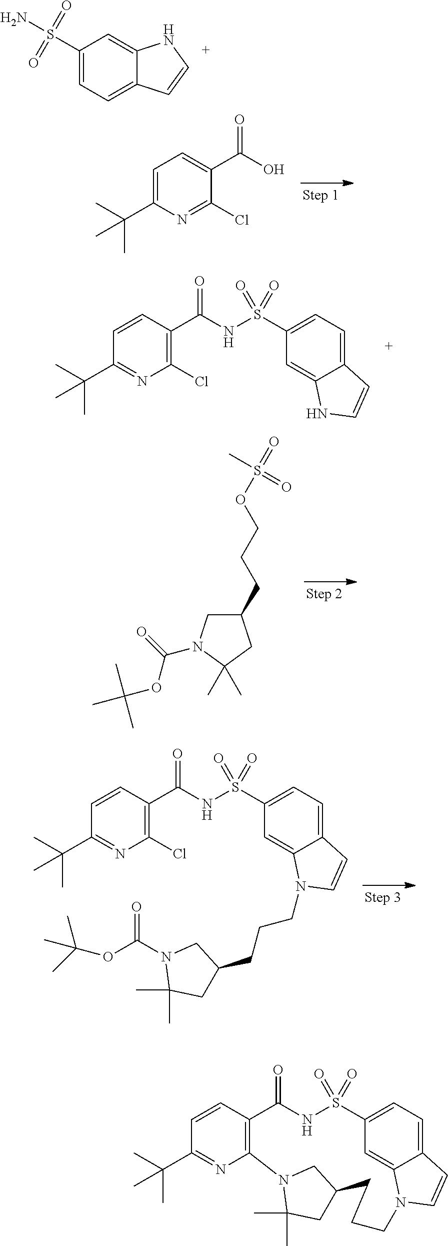 [SMILES: NS(=O)(=O)c1ccc2cc[nH]c2c1 ; CC(C)(C)c1ccc(C(=O)O)c(Cl)n1 ; CC(C)(C)c1ccc(C(=O)NS(=O)(=O)c2ccc3cc[nH]c3c2)c(Cl)n1 ; CC(C)(C)OC(=O)N1C[C@@H](CCCOS(C)(=O)=O)CC1(C)C ; CC(C)(C)OC(=O)N1C[C@@H](CCCn2ccc3ccc(S(=O)(=O)NC(=O)c4ccc(C(C)(C)C)nc4Cl)cc32)CC1(C)C ; CC(C)(C)c1ccc2c(n1)N1C[C@@H](CCCn3ccc4ccc(cc43)S(=O)(=O)NC2=O)CC1(C)C]

### Step 1: 6-tert-Butyl-2-chloro-N-(1H-indol-6-ylsulfonyl)pyridine-3-carboxamide

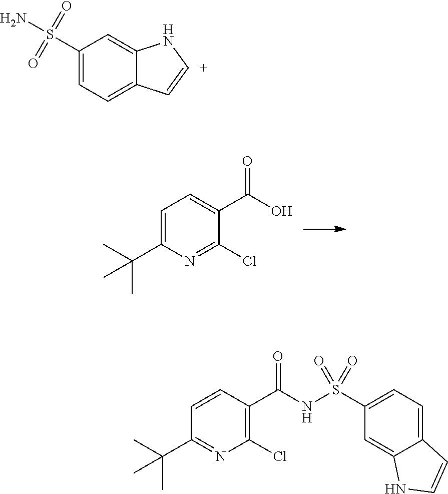 [SMILES: NS(=O)(=O)c1ccc2c(c1)N[C+]=C2 ; CC(C)(C)c1ccc(C(=O)O)c(Cl)n1 ; CC(C)(C)c1ccc(C(=O)NS(=O)(=O)c2ccc3cc[nH]c3c2)c(Cl)n1]

To a solution of the 6-tert-butyl-2-chloro-pyridine-3-carboxylic acid (108 mg, 0.5055 mmol) in THF (2 mL) was added CDI (87 mg, 0.5365 mmol) and the mixture was stirred at rt for 1 h then 1H-indole-6-sulfonamide (100 mg, 0.5096 mmol) was added followed by DBU (230 μL, 1.538 mmol) and the resulting mixture was stirred for 16 h at room temperature. The reaction mixture was quenched with a 1:1 mixture of saturated ammonium chloride and brine and was extracted with ethyl acetate. The organic phase was washed with a 1M citric acid solution, followed by brine. The organics were separated, dried over sodium sulfate, and evaporated. The resultant brown residue was purified by silica gel column chromatography using a shallow gradient of 100% hexanes to 100% EtOAc to afford 6-tert-butyl-2-chloro-N-(1H-indol-6-ylsulfonyl)pyridine-3-carboxamide (120 mg, 61%) as a white solid. ESI-MS m/z calc. 391.07574, found 392.2 (M+1)⁺; Retention time: 0.36 minutes (LC method I).

### Step 2: tert-Butyl (4S)-4-[3-[6-[(6-tert-butyl-2-chloro-pyridine-3-carbonyl)sulfamoyl]indol-1-yl]propyl]-2,2-dimethyl-pyrrolidine-1-carboxylate

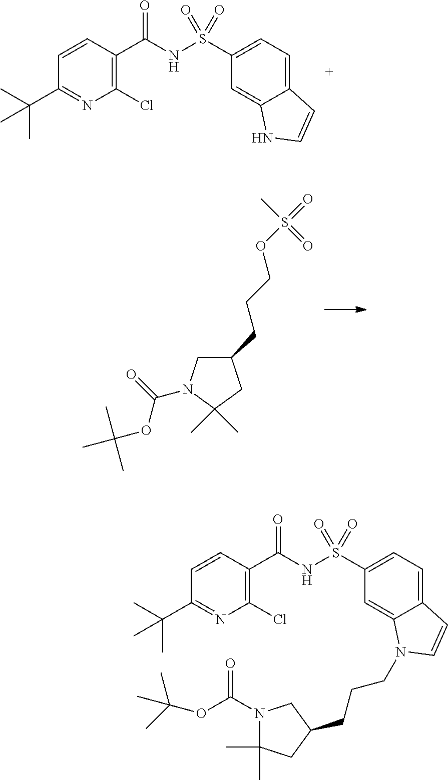 [SMILES: CC(C)(C)c1ccc(C(=O)NS(=O)(=O)c2ccc3cc[nH]c3c2)c(Cl)n1 ; CC(C)(C)OC(=O)N1C[C@@H](CCCOS(C)(=O)=O)CC1(C)C ; CC(C)(C)OC(=O)N1C[C@@H](CCCn2ccc3ccc(S(=O)(=O)NC(=O)c4ccc(C(C)(C)C)nc4Cl)cc32)CC1(C)C]

To a solution of tert-butyl (4S)-2,2-dimethyl-4-(3-methylsulfonyloxypropyl)pyrrolidine-1-carboxylate (104 mg, 0.3100 mmol) in DMF (3 mL) was added 6-tert-butyl-2-chloro-N-(1H-indol-6-ylsulfonyl)pyridine-3-carboxamide (120 mg, 0.3062 mmol) and cesium carbonate (308 mg, 0.9453 mmol) and the reaction mixture was heated at 70° C. for 24 h. The mixture was poured on crushed ice and the resultant white solid was collected by filtration and dried to afford tert-butyl (4S)-4-[3-[6-[(6-tert-butyl-2-chloro-pyridine-3-carbonyl)sulfamoyl]indol-1-yl]propyl]-2,2-dimethyl-pyrrolidine-1-carboxylate (152 mg, 78%). ESI-MS m/z calc. 630.2643, found 631.5 (M+1)⁺; Retention time: 0.7 minutes (LC method I).

### Step 3: (5S)-11-tert-Butyl-7,7-dimethyl-17λ⁶-thia-1,8,10,16-tetraazapentacyclo[16.5.2.15,8.09,14.021,24]hexacosa-9(14),10,12,18,20,22,24-heptaene-15,17,17-trione, Compound 260

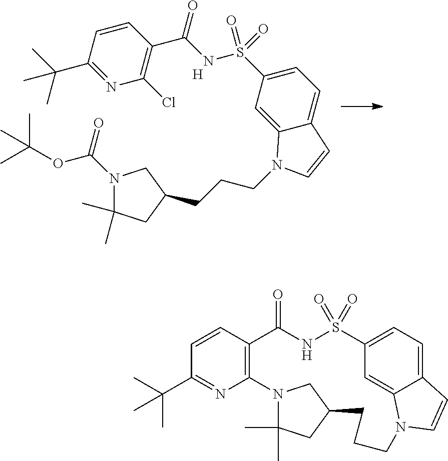

To a solution of tert-butyl (4S)-4-[3-[6-[(6-tert-butyl-2-chloro-pyridine-3-carbonyl)sulfamoyl]indol-1-yl]propyl]-2,2-dimethyl-pyrrolidine-1-carboxylate (150 mg, 0.2376 mmol) in NMP (2 mL) was added potassium carbonate (267 mg, 1.932 mmol). The mixture was purged with nitrogen for 5 min. The mixture was heated at 160° C. for 4 days. The mixture was cooled to room temperature, diluted with 1N HCl and extracted with EtOAc (1×). The organic phase was washed with brine (1×), dried (sodium sulfate), filtered and concentrated to a brown oil which was filtered and purified by reverse phase using a HPLC-MS method using a dual gradient run of 30-99% mobile phase B over 15.0 minutes (mobile phase A=H₂O (5 mM HCl), mobile phase B=acetonitrile) giving as an off-white solid, (5S)-11-tert-butyl-7,7-dimethyl-17λ⁶-thia-1,8,10,16-tetraazapentacyclo[16.5.2.15,8.09,14.021,24]hexacosa-9(14),10,12,18,20,22,24-heptaene-15,17,17-trione (5.8 mg, 5%). ¹H NMR (499 MHz, DMSO-d₆) δ 12.31 (s, 1H), 7.95 (d, J=1.5 Hz, 1H), 7.77 (d, J=8.4 Hz, 1H), 7.67-7.65 (m, 1H), 7.53 (d, J=7.9 Hz, 1H), 6.63 (d, J=7.9 Hz, 2H), 6.59 (d, J=3.1 Hz, 1H), 4.52 (dt, J=14.2, 4.8 Hz, 1H), 4.25-4.18 (m, 1H), 3.00 (t, J=7.8 Hz, 1H), 2.13 (d, J=11.5 Hz, 2H), 1.70-1.62 (m, 1H), 1.53 (s, 3H), 1.45-1.34 (m, 4H), 1.24 (d, J=6.6 Hz, 15H), 0.86 (dt, J=10.8, 7.2 Hz, 1H). ESI-MS m/z calc. 494.23517, found 495.14 (M+1)⁺; Retention time: 1.61 minutes (LC method G).

### Example 78: Preparation of (14S)-8-tert-Butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 296

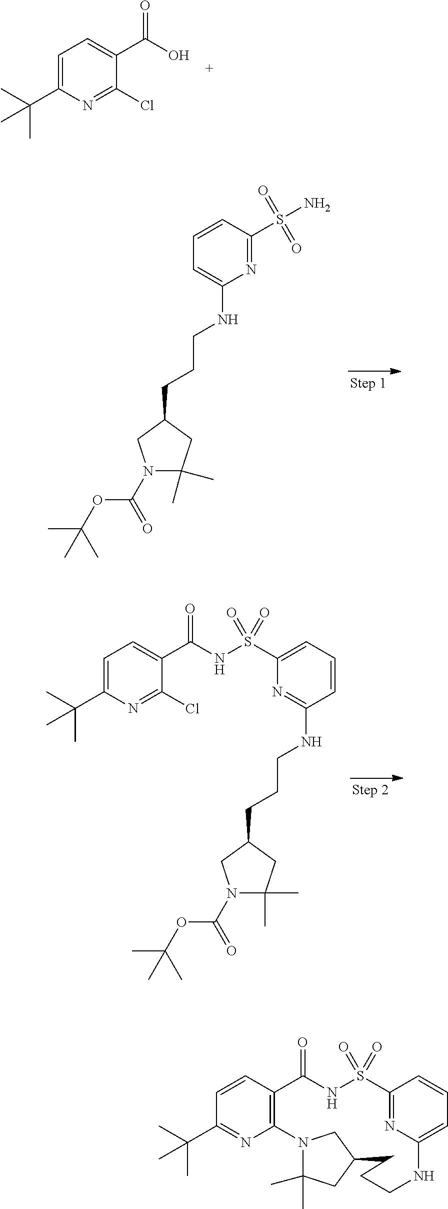 [SMILES: CC(C)(C)c1ccc(C(=O)O)c(Cl)n1 ; CC(C)(C)OC(=O)N1C[C@@H](CCCNc2cccc(S(N)(=O)=O)n2)CC1(C)C ; CC(C)(C)OC(=O)N1C[C@@H](CCCNc2cccc(S(=O)(=O)NC(=O)c3ccc(C(C)(C)C)nc3Cl)n2)CC1(C)C ; CC(C)(C)c1ccc2c(n1)N1C[C@@H](CCCNc3cccc(n3)S(=O)(=O)NC2=O)CC1(C)C]

### Step 1: tert-Butyl (4S)-4-[3-[[6-[(6-tert-butyl-2-chloro-pyridine-3-carbonyl)sulfamoyl]-2-pyridyl]amino]propyl]-2,2-dimethyl-pyrrolidine-1-carboxylate

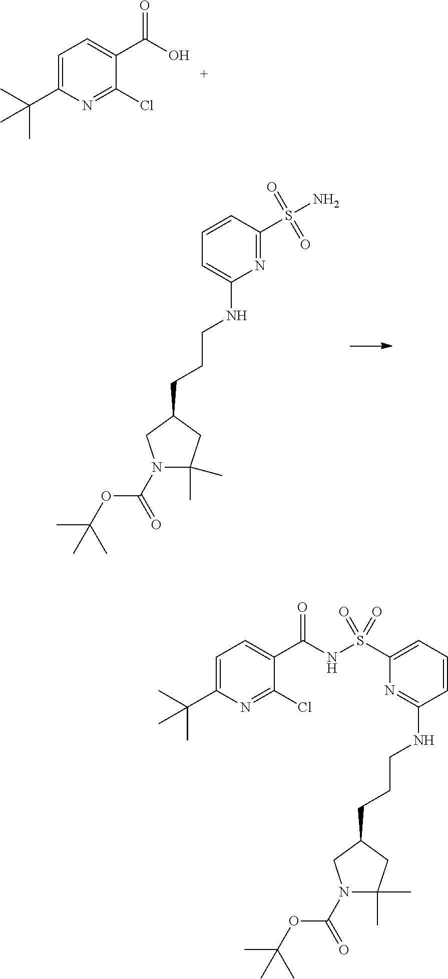 [SMILES: CC(C)(C)c1ccc(C(=O)O)c(Cl)n1 ; CC(C)(C)OC(=O)N1C[C@@H](CCCNc2cccc(S(N)(=O)=O)n2)CC1(C)C ; CC(C)(C)OC(=O)N1C[C@@H](CCCNc2cccc(S(=O)(=O)NC(=O)c3ccc(C(C)(C)C)nc3Cl)n2)CC1(C)C]

To a solution of 6-tert-butyl-2-chloro-pyridine-3-carboxylic acid (200 mg, 0.9361 mmol) in THF (8 mL) was added CDI (155 mg, 0.9559 mmol) (recrystallized from THF) and the mixture was stirred at rt for 2 h then tert-butyl (4S)-2,2-dimethyl-4-[3-[(6-sulfamoyl-2-pyridyl)amino]propyl]pyrrolidine-1-carboxylate (231 mg, 0.5599 mmol) was added followed by DBU (275 μL, 1.839 mmol) and the resulting mixture was stirred for 16 h at rt. The reaction was diluted with ethyl acetate and washed with a saturated aqueous sodium bicarbonate solution, and then the organic phase was extracted and washed with brine. The organics were separated, dried over sodium sulfate, evaporated and then purified on silica gel chromatography (80 gram column) using a gradient from 100% hexanes to 100% ethyl acetate followed by a second silica gel column (40 gram column) using a gradient from 100% dichloromethane to 15% methanol in dichloromethane to afford tert-butyl (4S)-4-[3-[[6-[(6-tert-butyl-2-chloro-pyridine-3-carbonyl)sulfamoyl]-2-pyridyl]amino]propyl]-2,2-dimethyl-pyrrolidine-1-carboxylate as an off-white solid (205 mg, 60%). ¹H NMR (500 MHz, DMSO-d₆) δ 12.68 (s, 1H), 7.89 (t, J=7.5 Hz, 1H), 7.61 (t, J=7.9 Hz, 1H), 7.52 (dd, J=8.0, 2.1 Hz, 1H), 7.18 (dd, J=18.9, 8.2 Hz, 2H), 6.74 (d, J=8.5 Hz, 1H), 3.53 (ddd, J=26.7, 10.5, 7.4 Hz, 1H), 3.37 (s, 2H), 3.25 (d, J=5.5 Hz, 2H), 2.76 (q, J=10.5 Hz, 1H), 2.14-1.99 (m, 1H), 1.84 (ddd, J=18.1, 12.0, 6.0 Hz, 1H), 1.49 (tt, J=12.8, 6.8 Hz, 2H), 1.37 (d, J=11.7 Hz, 9H), 1.34-1.30 (m, 4H), 1.28 (s, 9H), 1.21 (s, 3H). ESI-MS m/z calc. 607.2595, found 608.5 (M+1)⁺; Retention time: 2.27 minutes (LC method A).

### Step 2: (14S)-8-tert-Butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 296

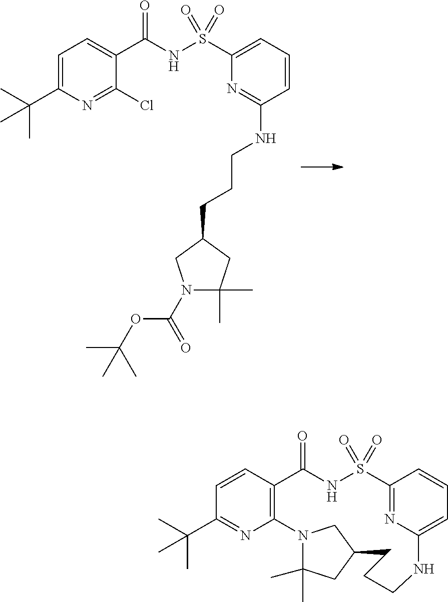 [SMILES: CC(C)(C)OC(=O)N1C[C@@H](CCCNc2cccc(S(=O)(=O)NC(=O)c3ccc(C(C)(C)C)nc3Cl)n2)CC1(C)C ; CC(C)(C)c1ccc2c(n1)N1C[C@@H](CCCNc3cccc(n3)S(=O)(=O)NC2=O)CC1(C)C]

Stage 1: tert-Butyl (4S)-4-[3-[[6-[(6-tert-butyl-2-chloro-pyridine-3-carbonyl)sulfamoyl]-2-pyridyl]amino]propyl]-2,2-dimethyl-pyrrolidine-1-carboxylate (195 mg, 0.3206 mmol) was dissolved in DCM (9 mL) and to the mixture was added HCl (4M in dioxane) (2.5 mL of 4 M, 10.00 mmol) and stirred at room temperature. The mixture was evaporated to dryness, then placed on the high vacuum pump for 2 h to afford the intermediate 6-tert-butyl-2-chloro-N-[[6-[3-[(3S)-5,5-dimethylpyrrolidin-3-yl]propylamino]-2-pyridyl]sulfonyl]pyridine-3-carboxamide (hydrochloride salt) ESI-MS m/z calc. 507.2071, found 508.2 (M+1)⁺; Retention time: 1.29 minutes (LC method A), as an off-white solid.

Stage 2: Combined material from Step 1 and K₂CO₃ (500 mg, 3.618 mmol), 3 Å molecular sieves and DMSO (6 mL) in a vial, purged with nitrogen, capped, heated to 165° C. and stirred for 60 h. Cooled to room temperature and the mixture was filtered and concentrated under a stream of nitrogen to give a residue which was purified by reverse-phase preparative chromatography utilizing a C₁₈ column and a 30-99% gradient over 15 min of acetonitrile in water (+5 mM HCl) to afford an off-white solid (14S)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (70 mg, 43%). ¹H NMR (500 MHz, DMSO-d₆) δ 12.45 (s, 1H), 7.63-7.52 (m, 2H), 7.05 (d, J=7.2 Hz, 2H), 6.72 (d, J=8.5 Hz, 1H), 6.63 (d, J=8.0 Hz, 1H), 3.91 (d, J=13.9 Hz, 1H), 3.07 (s, 1H), 2.93 (dt, J=13.7, 3.7 Hz, 1H), 2.73-2.59 (m, 1H), 2.10 (s, 1H), 1.80 (dd, J=11.9, 5.5 Hz, 2H), 1.61 (s, 3H), 1.55 (dd, J=31.2, 11.5 Hz, 3H), 1.48 (s, 3H), 1.30 (td, J=9.7, 8.1, 3.1 Hz, 1H), 1.27 (s, 9H). ESI-MS m/z calc. 471.2304, found 472.2 (M+1)⁺; Retention time: 2.02 minutes (LC method A).

### Example 79: Preparation of (18S)-11,11,16,16-tetramethyl-2λ⁶-thia-3,13,15,22,27-pentaazapentacyclo[21.3.1.115,18.05,14.07,12]octacosa-1(27),5(14),6,12,23,25-hexaene-2,2,4-trione, Compound 265

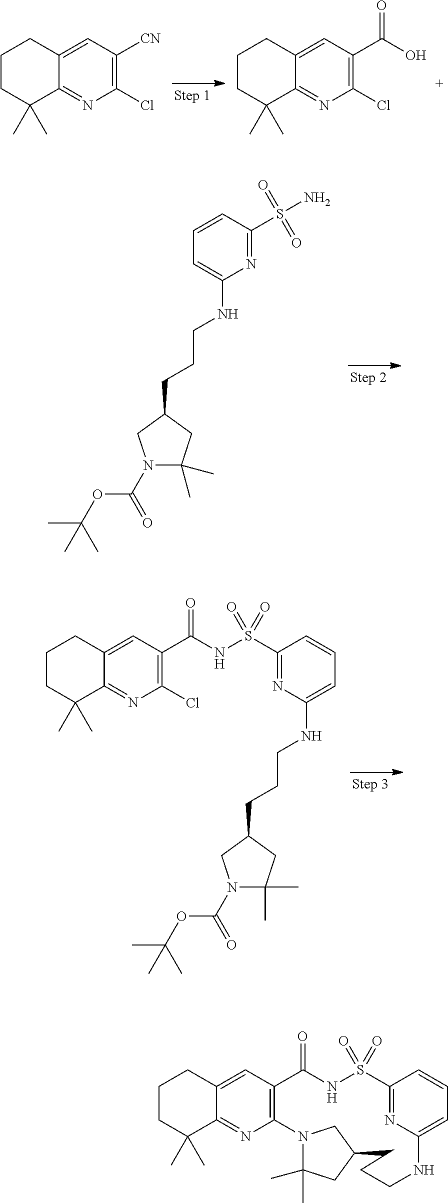 [SMILES: CC1(C)CCCc2cc(C#N)c(Cl)nc21 ; CC1(C)CCCc2cc(C(=O)O)c(Cl)nc21 ; CC(C)(C)OC(=O)N1C[C@@H](CCCNc2cccc(S(N)(=O)=O)n2)CC1(C)C ; CC(C)(C)OC(=O)N1C[C@@H](CCCNc2cccc(S(=O)(=O)NC(=O)c3cc4c(nc3Cl)C(C)(C)CCC4)n2)CC1(C)C ; CC1(C)CCCc2cc3c(nc21)N1C[C@@H](CCCNc2cccc(n2)S(=O)(=O)NC3=O)CC1(C)C]

### Step 1: 2-Chloro-8,8-dimethyl-6,7-dihydro-5H-quinoline-3-carboxylic acid

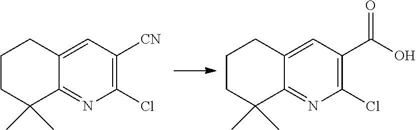 [SMILES: CC1(C)CCCc2cc(C#N)c(Cl)nc21 ; CC1(C)CCCc2cc(C(=O)O)c(Cl)nc21]

A solution of potassium hydroxide (3.81 g, 67.97 mmol) in water (12.50 mL) was added to a solution of 2-chloro-8,8-dimethyl-6,7-dihydro-5H-quinoline-3-carbonitrile (2.5 g, 11.33 mmol) in isopropanol (12.50 mL). The reaction mixture was allowed to stir in a pre-heated 90° C. oil bath overnight. After cooling to room temperature, the reaction mixture was concentrated and acidified to pH˜3. The precipitate was filtered, washed with water and dried in a vacuum oven overnight to give a white solid. 2-Chloro-8,8-dimethyl-6,7-dihydro-5H-quinoline-3-carboxylic acid (2.5 g, 91%). ¹H NMR (400 MHz, DMSO) δ 13.45 (s, 1H), 7.89 (s, 1H), 2.75 (s, 2H), 1.73 (s, 4H), 1.25 (s, 6H). ESI-MS m/z calc. 239.0713, found 240.0 (M+1)⁺; Retention time: 1.48 minutes (LC method A).

### Step 2: tert-Butyl (4S)-4-[3-[[6-[(2-chloro-8,8-dimethyl-6,7-dihydro-5H-quinoline-3-carbonyl)sulfamoyl]-2-pyridyl]amino]propyl]-2,2-dimethyl-pyrrolidine-1-carboxylate

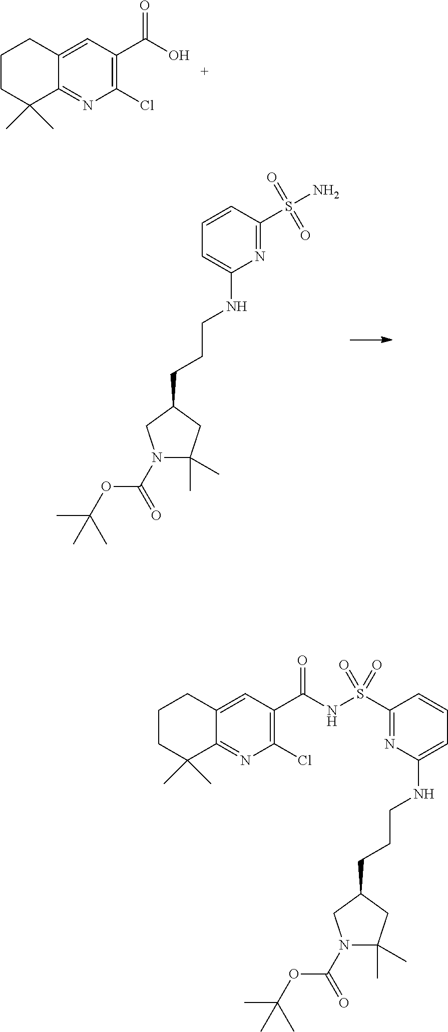 [SMILES: CC1(C)CCCc2cc(C(=O)O)c(Cl)nc21 ; CC(C)(C)OC(=O)N1C[C@@H](CCCNc2cccc(S(N)(=O)=O)n2)CC1(C)C ; CC(C)(C)OC(=O)N1C[C@@H](CCCNc2cccc(S(=O)(=O)NC(=O)c3cc4c(nc3Cl)C(C)(C)CCC4)n2)CC1(C)C]

To a solution of 2-chloro-8,8-dimethyl-6,7-dihydro-5H-quinoline-3-carboxylic acid (95 mg, 0.3963 mmol) in THF (4 mL) was added CDI (65 mg, 0.4009 mmol) and the mixture was stirred at rt for 14 h then tert-butyl (4S)-2,2-dimethyl-4-[3-[(6-sulfamoyl-2-pyridyl)amino]propyl]pyrrolidine-1-carboxylate (125 mg, 0.3030 mmol) was added followed by DBU (150 μL, 1.003 mmol) and the resulting mixture was stirred for 16 h at rt. The reaction was diluted with ethyl acetate and washed with a saturated aqueous sodium bicarbonate solution, and then the organic phase was extracted and washed with brine. The organics were separated, dried over sodium sulfate, evaporated and then the residue was purified by reverse-phase preparative chromatography utilizing a C₁₈ column and a 30-80% gradient over 30 min of acetonitrile in water (+5 mM HCl) to afford tert-butyl (4S)-4-[3-[[6-[(2-chloro-8,8-dimethyl-6,7-dihydro-5H-quinoline-3-carbonyl)sulfamoyl]-2-pyridyl]amino]propyl]-2,2-dimethyl-pyrrolidine-1-carboxylate (86 mg, 45%). ESI-MS m/z calc. 633.27515, found 634.2 (M+1)⁺; Retention time: 2.29 minutes as an off-white solid (LC method A).

### Step 3: (18S)-11,11,16,16-Tetramethyl-2λ⁶-thia-3,13,15,22,27-pentaazapentacyclo[21.3.1.115,18.05,14.07,12]octacosa-1(27),5(14),6,12,23,25-hexaene-2,2,4-trione, Compound 265

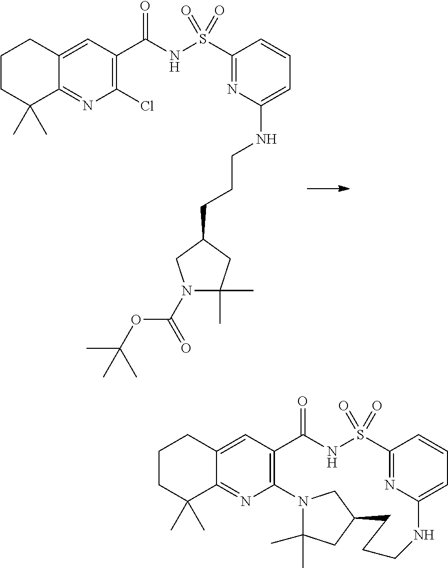 [SMILES: CC(C)(C)OC(=O)N1C[C@@H](CCCNc2cccc(S(=O)(=O)NC(=O)c3cc4c(nc3Cl)C(C)(C)CCC4)n2)CC1(C)C ; CC1(C)CCCc2cc3c(nc21)N1C[C@@H](CCCNc2cccc(n2)S(=O)(=O)NC3=O)CC1(C)C]

Stage 1: tert-Butyl (4S)-4-[3-[[6-[(2-chloro-8,8-dimethyl-6,7-dihydro-5H-quinoline-3-carbonyl)sulfamoyl]-2-pyridyl]amino]propyl]-2,2-dimethyl-pyrrolidine-1-carboxylate (86 mg, 0.1356 mmol) was dissolved in DCM (3 mL) and to the mixture was added TFA (500 μL, 6.490 mmol) and the reaction was stirred at room temperature. After 2 h, the mixture was evaporated to dryness, then diluted with diethyl ether (30 mL×2), and reconcentrated. The material was then placed on the high vacuum pump for 2 h to afford the intermediate 2-chloro-N-[[6-[3-[(3S)-5,5-dimethylpyrrolidin-3-yl]propylamino]-2-pyridyl]sulfonyl]-8,8-dimethyl-6,7-dihydro-5H-quinoline-3-carboxamide ESI-MS m/z calc. 533.2227, found 534.2 (M+1)⁺; Retention time: 1.34 minutes as a light brown oil. Stage 2: Combined material from Step 1 and K₂CO₃ (205 mg, 1.483 mmol), 3 Å molecular sieves and NMP (4 mL) in a vial, purged with nitrogen, capped, heated to 170° C. and stirred for 84 h. Cooled to room temperature and the mixture was diluted with ethyl acetate and water. The organic layer was extracted (2×) and was further washed with 10% citric acid solution followed by brine. The organics were separated, dried over sodium sulfate, evaporated to a light brown oil. This residue was purified by reverse-phase preparative chromatography utilizing a C₁₈ column and a 10-99% gradient over 15 min of acetonitrile in water (+5 mM HCl) to afford as a white solid (18S)-11,11,16,16-tetramethyl-2λ⁶-thia-3,13,15,22,27-pentaazapentacyclo[21.3.1.115,18.05,14.07,12]octacosa-1(27),5(14),6,12,23,25-hexaene-2,2,4-trione (7.79 mg, 11%). ¹H NMR (499 MHz, DMSO-d₆) δ 12.34 (s, 1H), 7.56 (dd, J=8.5, 7.1 Hz, 1H), 7.30 (s, 1H), 7.04 (d, J=7.1 Hz, 1H), 6.95 (d, J=8.9 Hz, 1H), 6.70 (d, J=8.4 Hz, 1H), 3.91 (d, J=11.7 Hz, 1H), 3.08 (s, 1H), 2.98-2.89 (m, 1H), 2.69-2.62 (m, 2H), 2.58-2.52 (m, 1H), 2.14-2.03 (m, 1H), 1.79 (dd, J=11.7, 5.4 Hz, 1H), 1.70 (dt, J=14.9, 4.8 Hz, 4H), 1.67-1.62 (m, 1H), 1.59 (s, 3H), 1.56 (dd, J=11.7, 4.7 Hz, 2H), 1.50 (d, J=12.3 Hz, 1H), 1.46 (s, 3H), 1.29 (s, 3H), 1.25 (dd, J=13.8, 4.9 Hz, 1H), 1.15 (s, 3H). ESI-MS m/z calc. 497.24606, found 498.2 (M+1)⁺; Retention time: 2.04 minutes (LC method A).

### Example 80: Preparation of (14S)-8-tert-butyl-12,12-dimethyl-2λ⁶-thia-3,7,9,11,18,23-hexaazatetracyclo[17.3.1.111,14.05,10]tetracosa-1(22),5,7,9,19(23),20-hexaene-2,2,4-trione, Compound 11

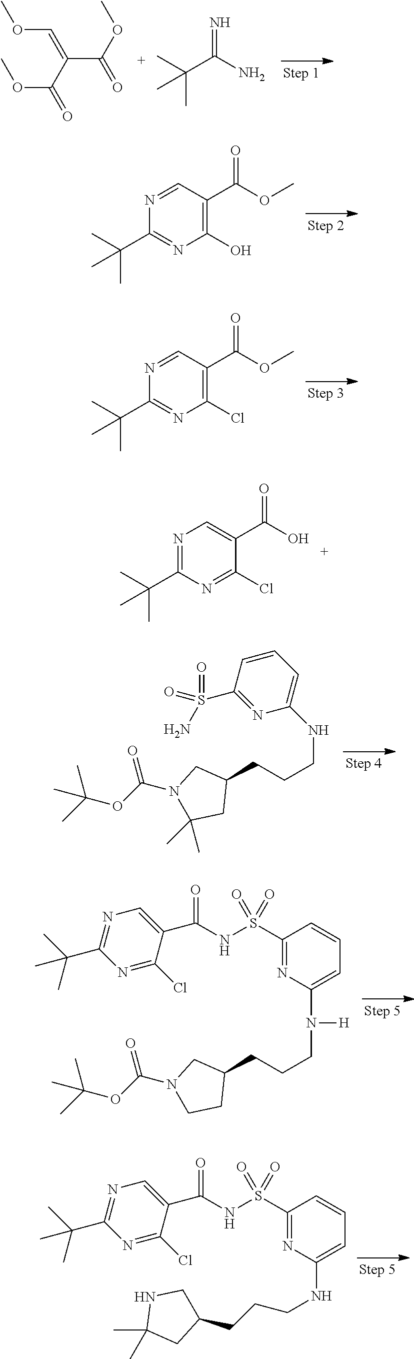

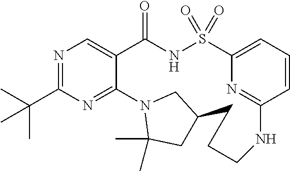 [SMILES: CC(C)(C)c1ncc2c(n1)N1C[C@@H](CCCNc3cccc(n3)S(=O)(=O)NC2=O)CC1(C)C]

### Step 1: Methyl 2-tert-butyl-4-hydroxy-pyrimidine-5-carboxylate

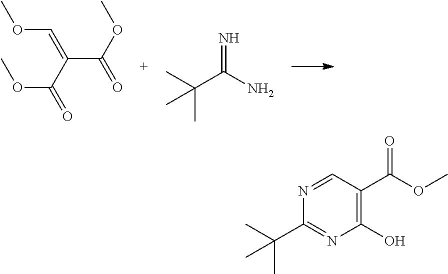

To a solution of 2,2-dimethylpropanamidine (hydrochloride salt) (35 g, 256.18 mmol) in MeOH (560 mL) was added sodium methoxide (14 g, 259.15 mmol) and the solution was stirred at room temperature for 15 min. To this was added a solution of dimethyl 2-(methoxymethylene)propanedioate (44.6 g, 256.10 mmol) in MeOH (20 mL) and the solution was stirred at room temperature for 45 min then at reflux for 1 h. The suspension was cooled to room temperature and concentrated. The oily mass was dissolved in H₂O (875 mL) and the pH adjusted to 3 (pH paper) with AcOH. The precipitated solids were collected by filtration, washed with H₂O (250 mL) and dried. Afforded methyl 2-tert-butyl-4-hydroxy-pyrimidine-5-carboxylate (25.18 g, 47%) as a white solid. ESI-MS m/z calc. 210.1004, found 211.2 (M+1)⁺; Retention time: 1.32 minutes The aqueous filtrate was extracted with DCM (6×30 mL). The combined organic layers were washed with brine, dried over sodium sulfate, filtered and concentrated under reduced pressure. The resulting semi-solid was triturated from MTBE (60 mL). Afforded methyl 2-tert-butyl-4-hydroxy-pyrimidine-5-carboxylate (4.65 g, 9%) as a white solid. ESI-MS m/z calc. 210.1004, found 211.2 (M+1)⁺; Retention time: 1.32 minutes (LC method E).

### Step 2: Methyl 2-tert-butyl-4-chloro-pyrimidine-5-carboxylate

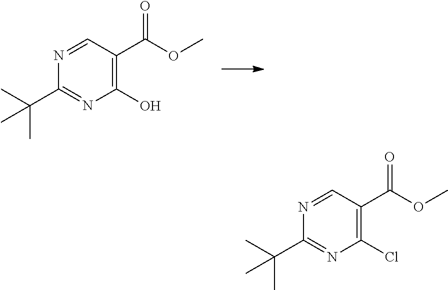

To ice cold (0-5° C.) phosphorous oxychloride (20.892 g, 12.7 mL, 136.25 mmol) was added dropwise triethylamine (166.98 mg, 230 μL, 1.6502 mmol) followed by methyl 2-tert-butyl-4-hydroxy-pyrimidine-5-carboxylate (20.5 g, 97.415 mmol). The mixture was then warmed to 80° C. for 5 h. The reaction mixture was cooled down to room temperature, concentrated under reduced pressure, diluted with DCM (110 mL) and poured slowly onto ice (˜440 g) and water (90 mL). The resulting mixture was stirred at room temperature until the ice all melted. The organic phase was separated and the aqueous phase was extracted with DCM (2×50 mL). Combined organic layers were washed with sodium bicarbonate (100 mL), brine (150 mL), dried over sodium sulfate, filtered and concentrated under reduced pressure. Afforded methyl 2-tert-butyl-4-chloro-pyrimidine-5-carboxylate (21.2 g, 95%) as a yellow solid. ¹H NMR (400 MHz, DMSO-d₆) δ 9.14 (s, 1H), 3.90 (s, 3H), 1.35 (s, 9H). ESI-MS m/z calc. 228.0666, found 229.1 (M+1)⁺; Retention time: 4.49 minutes (LC method F).

### Step 3: 2-tert-Butyl-4-chloro-pyrimidine-5-carboxylic acid

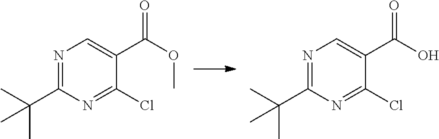

To a solution of methyl 2-tert-butyl-4-chloro-pyrimidine-5-carboxylate (10.96 g, 47.928 mmol) in THF (120 mL) and Water (120 mL) was added lithium hydroxide hydrate (3 g, 71.491 mmol) and the reaction was stirred at room temperature for 0.5 h. 1 N aqueous HCl was added until pH=2 was reached and the volatiles were removed under reduced pressure. The product was then extracted using a mix CHCl₃/MeOH 3:1 v/v (4×100 mL). Combined organic layers were dried over sodium sulfate, filtered and concentrated under reduced pressure to afford 2-tert-butyl-4-chloro-pyrimidine-5-carboxylic acid (10.1 g, 96%) as a white solid. ¹H NMR (400 MHz, DMSO-d₆) δ 13.95 (br. s., 1H), 9.11 (s, 1H), 1.35 (s, 9H). ESI-MS m/z calc. 214.0509, found 215.1 (M+1)⁺; Retention time: 4.11 minutes (LC method A).

### Step 4: tert-Butyl (4S)-4-[3-[[6-[(2-tert-butyl-4-chloro-pyrimidine-5-carbonyl)sulfamoyl]-2-pyridyl]amino]propyl]-2,2-dimethyl-pyrrolidine-1-carboxylate

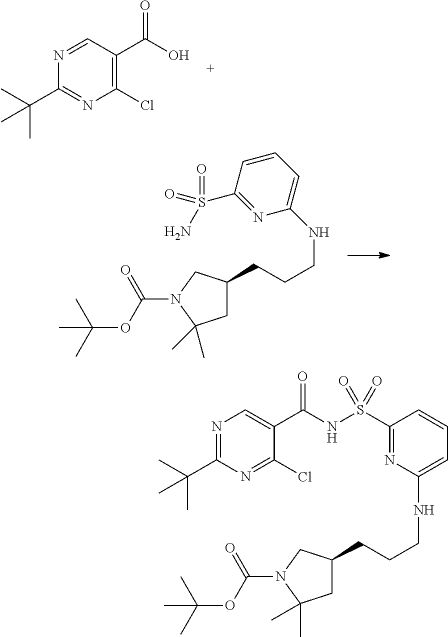 [SMILES: CC(C)(C)c1ncc(C(=O)O)c(Cl)n1 ; CC(C)(C)OC(=O)N1C[C@@H](CCCNc2cccc(S(N)(=O)=O)n2)CC1(C)C ; CC(C)(C)OC(=O)N1C[C@@H](CCCNc2cccc(S(=O)(=O)NC(=O)c3cnc(C(C)(C)C)nc3Cl)n2)CC1(C)C]

A round bottom flask was charged under nitrogen with 2-tert-butyl-4-chloro-pyrimidine-5-carboxylic acid (500 mg, 2.3294 mmol) and tetrahydrofuran (4 mL). 1,1′-Carbonyldiimidazole (380 mg, 2.3435 mmol) was added and the mixture was stirred under nitrogen at room temperature for 2 h (complete conversion by LCMS of aliquot quenched in ammonium hydroxide). In a separate flask, a solution of tert-butyl (4S)-2,2-dimethyl-4-[3-[(6-methylsulfonyl-2-pyridyl)amino]propyl]pyrrolidine-1-carboxylate (300 mg, 0.7272 mmol) in anhydrous tetrahydrofuran (2 mL) was prepared under nitrogen atmosphere and it was subsequently added via syringe into the activated solution. 1,8-Diazabicyclo[5.4.0]undec-7-ene (0.3 mL, 2.0061 mmol) was added through syringe and the reaction mixture was stirred at room temperature under nitrogen atmosphere for 2 h. After, the solvents were removed under reduced pressure and the resulting thick oil was treated with ethyl acetate (40 mL) and water (25 mL). The aqueous phase was extracted with ethyl acetate (30 mL). The combined organic layers were washed with aqueous solution of HCl 1 N (20 mL), brine (20 mL) and dried over sodium sulfate. The crude was directly purified by reverse phase chromatography using a 80 g cartridge, eluting with a gradient of MeCN in water (containing 0.1% of formic acid) (50% for 5 column volumes then 50 to 100% in 20 CV) to afford tert-butyl (4S)-4-[3-[[6-[(2-tert-butyl-4-chloro-pyrimidine-5-carbonyl)sulfamoyl]-2-pyridyl]amino]propyl]-2,2-dimethyl-pyrrolidine-1-carboxylate (850 mg, 59%) as a light yellow solid. ¹H NMR (400 MHz, DMSO-d₆) δ 12.80 (br. s., 1H), 8.89 (s, 1H), 7.61 (t, J=7.8 Hz, 1H), 7.21 (br. s., 1H), 7.16 (d, J=7.3 Hz, 1H), 6.74 (d, J=8.6 Hz, 1H), 3.58-3.45 (m, 1H), 3.24-3.20 (m, 2H), 2.76 (q, J=10.2 Hz, 1H), 2.12-1.98 (m, 1H), 1.89-1.82 (m, 1H), 1.57-1.44 (m, 2H), 1.43-1.26 (m, 24H), 1.22 (s, 3H). ESI-MS m/z calc. 608.2548, found 553.2 (M−55)+; Retention time: 2.32 minutes (LC method E).

### Step 5: 2-tert-Butyl-4-chloro-N-[[6-[3-[(3S)-5,5-dimethylpyrrolidin-3-yl]propylamino]-2-pyridyl]sulfonyl]pyrimidine-5-carboxamide

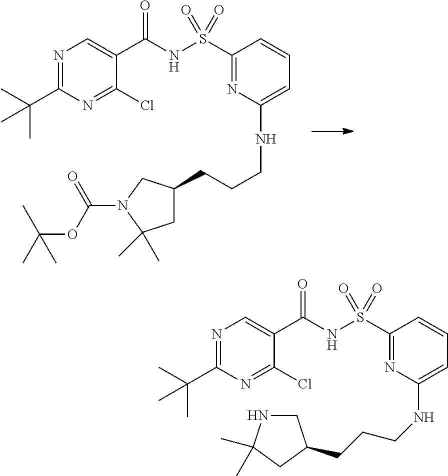 [SMILES: CC(C)(C)OC(=O)N1C[C@@H](CCCNc2cccc(S(=O)(=O)NC(=O)c3cnc(C(C)(C)C)nc3Cl)n2)CC1(C)C ; CC1(C)C[C@H](CCCNc2cccc(S(=O)(=O)NC(=O)c3cnc(C(C)(C)C)nc3Cl)n2)CN1]

In a round bottom flask, tert-butyl (4S)-4-[3-[[6-[(2-tert-butyl-4-chloro-pyrimidine-5-carbonyl)sulfamoyl]-2-pyridyl]amino]propyl]-2,2-dimethyl-pyrrolidine-1-carboxylate (250 mg, 0.4042 mmol) was stirred at room temperature in dichloromethane (2 mL) and HCl (1.5 mL of 4 M in dioxane, 6.0000 mmol) for 2 h. The solvent was evaporated to afford 2-tert-butyl-4-chloro-N-[[6-[3-[(3S)-5,5-dimethylpyrrolidin-3-yl]propylamino]-2-pyridyl]sulfonyl]pyrimidine-5-carboxamide (dihydrochloride salt) (250 mg, 91%) as a yellow solid. ESI-MS m/z calc. 508.2023, found 509.2 (M+1)⁺; Retention time: 1.44 minutes (LC method E).

### Step 6: (14S)-8-tert-butyl-12,12-dimethyl-2λ⁶-thia-3,7,9,11,18,23-hexaazatetracyclo[17.3.1.111,14.05,10]tetracosa-1(22),5,7,9,19(23),20-hexaene-2,2,4-trione, Compound 11

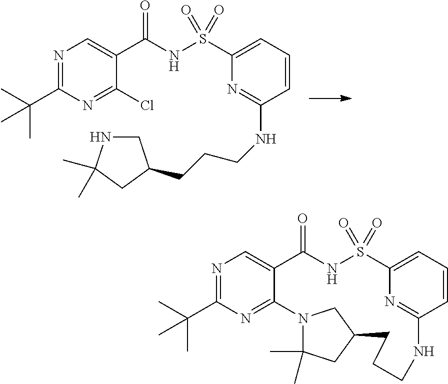 [SMILES: CC1(C)C[C@H](CCCNc2cccc(S(=O)(=O)NC(=O)c3cnc(C(C)(C)C)nc3Cl)n2)CN1 ; CC(C)(C)c1ncc2c(n1)N1C[C@@H](CCCNc3cccc(n3)S(=O)(=O)NC2=O)CC1(C)C]

2-tert-Butyl-4-chloro-N-[[6-[3-[(3S)-5,5-dimethylpyrrolidin-3-yl]propylamino]-2-pyridyl]sulfonyl]pyrimidine-5-carboxamide (dihydrochloride salt) (250 mg, 0.3694 mmol) was dissolved in N-methyl-2-pyrrolidone (NMP) (3 mL). Potassium carbonate (320 mg, 2.3154 mmol) and cesium fluoride (60 mg, 0.3950 mmol) were added. The tube was sealed and the mixture was heated at 150° C. for 2 h. The reaction mixture was directly purified two times by reverse phase chromatography (C₁₈ 50 g) using gradients of 5 to 100% acetonitrile in water and 5 to 100% acetonitrile in water (containing 0.1% of formic acid) to afford (14S)-8-tert-butyl-12,12-dimethyl-2λ⁶-thia-3,7,9,11,18,23-hexaazatetracyclo[17.3.1.111,14.05,10]tetracosa-1(22),5,7,9,19(23),20-hexaene-2,2,4-trione (23 mg, 13%) as a pale brown solid. ¹H NMR (400 MHz, DMSO-d₆, 80° C.) δ 8.29 (s, 1H), 7.59-7.55 (m, 1H), 7.09 (d, J=7.3 Hz, 1H), 6.82 (br. s., 1H), 6.73 (d, J=8.3 Hz, 1H), 3.88-3.79 (m, 2H), 3.42-3.28 (m, 2H), 3.06-3.00 (m, 1H), 2.83 (t, J=10.5 Hz, 1H), 2.23-2.11 (m, 1H), 1.93-1.89 (m, 1H), 1.85-1.74 (m, 1H), 1.68-1.53 (m, 9H), 1.34 (s, 9H). ESI-MS m/z calc. 472.2257, found 473.2 (M+1)⁺; Retention time: 3.03 minutes (LC method F).

### Example 81: Preparation of (14S)-8-tert-Butyl-17-(4-tert-butylpyridin-2-yl)-12,12-dimethyl-2λ⁶-thia-3,7,9,11,18,23-hexaazatetracyclo[17.3.1.111,14.05,10]tetracosa-1(22),5,7,9,19(23),20-hexaene-2,2,4-trione, Compound 2 (diastereomer 1), and Compound 1 (diastereomer 2)

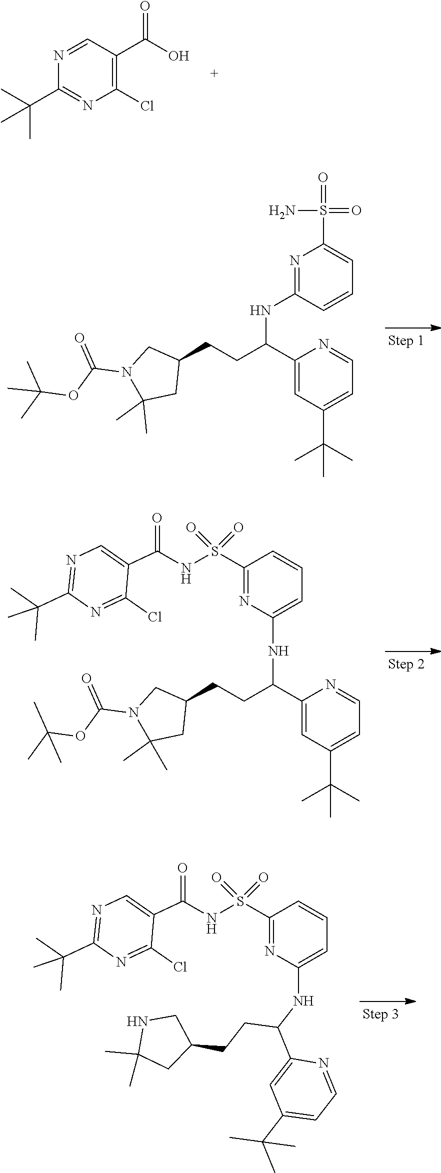

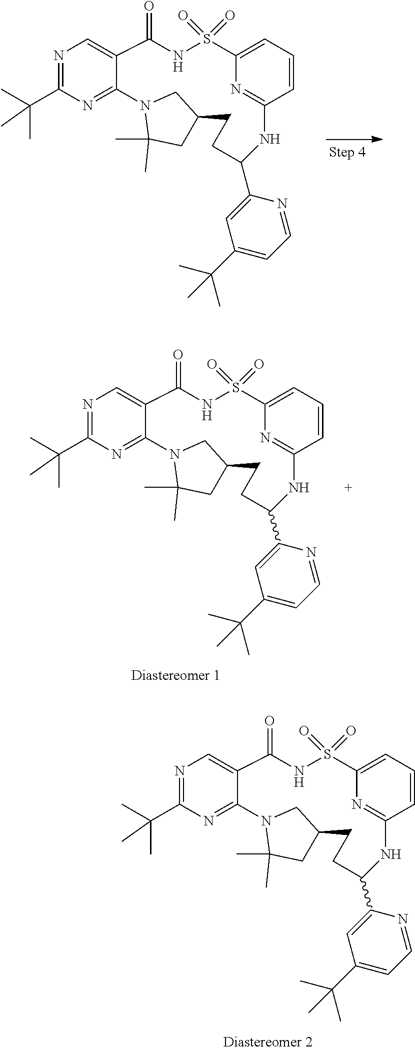 [SMILES: CC(C)(C)c1ccnc(C2CC[C@@H]3CN(c4nc(C(C)(C)C)ncc4C(=O)NS(=O)(=O)c4cccc(n4)N2)C(C)(C)C3)c1 ; CC(C)(C)c1ccnc(C2CC[C@@H]3CN(c4nc(C(C)(C)C)ncc4C(=O)NS(=O)(=O)c4cccc(n4)N2)C(C)(C)C3)c1 ; CC(C)(C)c1ccnc(C2CC[C@@H]3CN(c4nc(C(C)(C)C)ncc4C(=O)NS(=O)(=O)c4cccc(n4)N2)C(C)(C)C3)c1]

### Step 1: tert-Butyl (4S)-4-[3-[[6-[(2-tert-butyl-4-chloro-pyrimidine-5-carbonyl)sulfamoyl]-2-pyridyl]amino]-3-(4-tert-butyl-2-pyridyl)propyl]-2,2-dimethyl-pyrrolidine-1-carboxylate

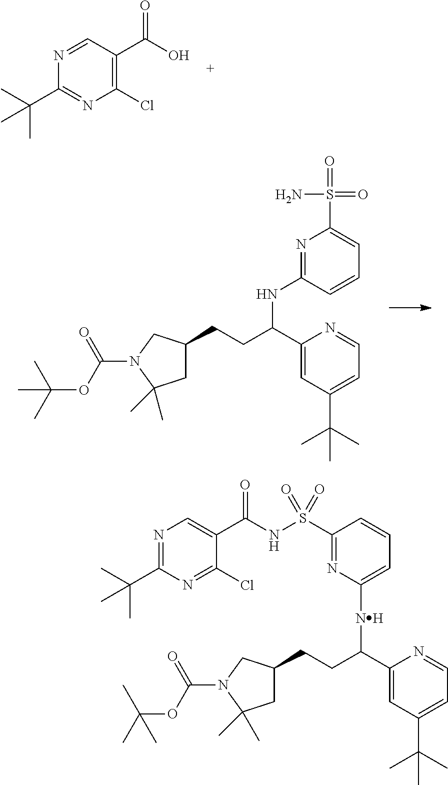

A round bottom flask was charged under nitrogen with 2-tert-butyl-4-chloro-pyrimidine-5-carboxylic acid (284 mg, 1.3231 mmol) and tetrahydrofuran (8 mL). 1,1′-Carbonyldiimidazole (271 mg, 1.6713 mmol) was added and the mixture was stirred under nitrogen at room temperature for 3 h30. In a separate flask, a solution of tert-butyl (4S)-4-[3-(4-tert-butyl-2-pyridyl)-3-[(6-sulfamoyl-2-pyridyl)amino]propyl]-2,2-dimethyl-pyrrolidine-1-carboxylate (731 mg, 1.3395 mmol) in tetrahydrofuran (5.4 mL) was prepared under nitrogen atmosphere and it was subsequently added into the activated solution. 1,8-Diazabicyclo[5.4.0]undec-7-ene (578.22 mg, 568 μL, 3.7982 mmol) was added and the reaction mixture was stirred at room temperature under nitrogen atmosphere for 3 h. The solvents were removed under reduced pressure and the resulting thick oil was treated with ethyl acetate (30 mL) and water (20 mL). The aqueous phase was extracted with ethyl acetate (20 mL). The combined organic layers were washed with an aqueous solution of HCl 1 N (20 mL) and dried over sodium sulfate. The crude product was purified by reverse phase chromatography on C₁₈ (80 g, eluting 50 to 100% acetonitrile in water). The product fractions were combined and the organic solvents were removed under reduced pressure. The remaining aqueous mixture was extracted with ethyl acetate (2×25 mL). The combined organic layers were dried over anhydrous sodium sulfate, filtered and concentrated under reduced pressure to give tert-butyl (4S)-4-[3-[[6-[(2-tert-butyl-4-chloro-pyrimidine-5-carbonyl)sulfamoyl]-2-pyridyl]amino]-3-(4-tert-butyl-2-pyridyl)propyl]-2,2-dimethyl-pyrrolidine-1-carboxylate (516 mg, 53%) as a beige solid. ¹H NMR (400 MHz, DMSO-d₆) δ 8.77 (d, J=4.2 Hz, 1H), 8.40 (d, J=5.4 Hz, 1H), 7.46 (t, J=7.8 Hz, 1H), 7.41 (s, 1H), 7.28-7.10 (m, 2H), 7.02 (d, J=7.1 Hz, 1H), 6.60 (br. s., 1H), 5.03 (br. s., 1H), 3.94-3.55 (m, 1H), 3.52-3.38 (m, 1H), 2.74-2.60 (m, 1H), 2.04-1.90 (m, 2H), 1.88-1.67 (m, 3H), 1.42-1.12 (m, 35H). ESI-MS m/z calc. 741.3439, found 742.2 (M+1)⁺; Retention time: 2.07 minutes (LC method E).

### Step 2: 2-tert-Butyl-N-[[6-[[1-(4-tert-butyl-2-pyridyl)-3-[(3S)-5,5-dimethylpyrrolidin-3-yl]propyl]amino]-2-pyridyl]sulfonyl]-4-chloro-pyrimidine-5-carboxamide

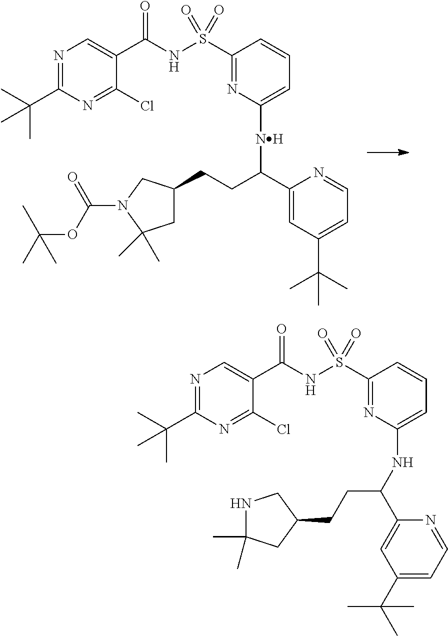

HCl (in dioxane) (2.2 mL of 4 M, 8.8000 mmol) was added to a solution of tert-butyl (4S)-4-[3-[[6-[(2-tert-butyl-4-chloro-pyrimidine-5-carbonyl)sulfamoyl]-2-pyridyl]amino]-3-(4-tert-butyl-2-pyridyl)propyl]-2,2-dimethyl-pyrrolidine-1-carboxylate (444 mg, 0.5981 mmol) in dichloromethane (7 mL). The reaction mixture was stirred at room temperature for 3 h. The solvent was evaporated to afford 2-tert-butyl-N-[[6-[[1-(4-tert-butyl-2-pyridyl)-3-[(3S)-5,5-dimethylpyrrolidin-3-yl]propyl]amino]-2-pyridyl]sulfonyl]-4-chloro-pyrimidine-5-carboxamide (trihydrochloride salt) (495 mg, 80%) as a beige solid. ESI-MS m/z calc. 641.2915, found 642.2 (M+1)⁺; Retention time: 1.4 minutes (LC method E). The crude was used for the next step without further purification.

### Step 3: (14S)-8-tert-Butyl-17-(4-tert-butylpyridin-2-yl)-12,12-dimethyl-2λ⁶-thia-3,7,9,11,18,23-hexaazatetracyclo[17.3.1.111,14.05,10]tetracosa-1(22),5,7,9,19(23),20-hexaene-2,2,4-trione

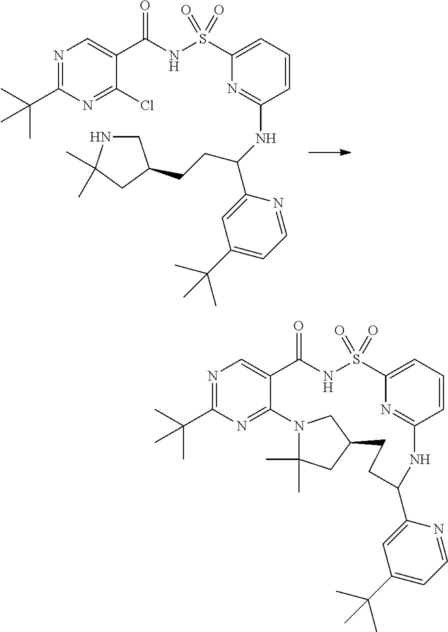 [SMILES: CC1(C)C[C@H](CCC(Nc2cccc(S(=O)(=O)NC(=O)c3cnc(C(C)(C)C)nc3Cl)n2)c2cc(C(C)(C)C)ccn2)CN1 ; CC(C)(C)c1ccnc(C2CC[C@@H]3CN(c4nc(C(C)(C)C)ncc4C(=O)NS(=O)(=O)c4cccc(n4)N2)C(C)(C)C3)c1]

2-tert-Butyl-N-[[6-[[1-(4-tert-butyl-2-pyridyl)-3-[(3S)-5,5-dimethylpyrrolidin-3-yl]propyl]amino]-2-pyridyl]sulfonyl]-4-chloro-pyrimidine-5-carboxamide (trihydrochloride salt) (495 mg, 0.4801 mmol) was dissolved in N-methyl-2-pyrrolidone (10 mL). Potassium carbonate (516 mg, 3.7336 mmol) and cesium fluoride (97 mg, 0.6386 mmol) were added. The mixture was heated at 150° C. for 2 h. The reaction mixture was combined with another reaction mixture run on a 50 mg scale and worked-up in the next step.

### Step 4: (14S)-8-tert-Butyl-17-(4-tert-butylpyridin-2-yl)-12,12-dimethyl-2λ⁶-thia-3,7,9,11,18,23-hexaazatetracyclo[17.3.1.111,14.05,10]tetracosa-1(22),5,7,9,19(23),20-hexaene-2,2,4-trione, Compound 2 (diastereomer 1), and (14S)-8-tert-butyl-17-(4-tert-butylpyridin-2-yl)-12,12-dimethyl-2λ⁶-thia-3,7,9,11,18,23-hexaazatetracyclo[17.3.1.111,14.05,10]tetracosa-1(22),5,7,9,19(23),20-hexaene-2,2,4-trione, Compound 1 (diastereomer 2)

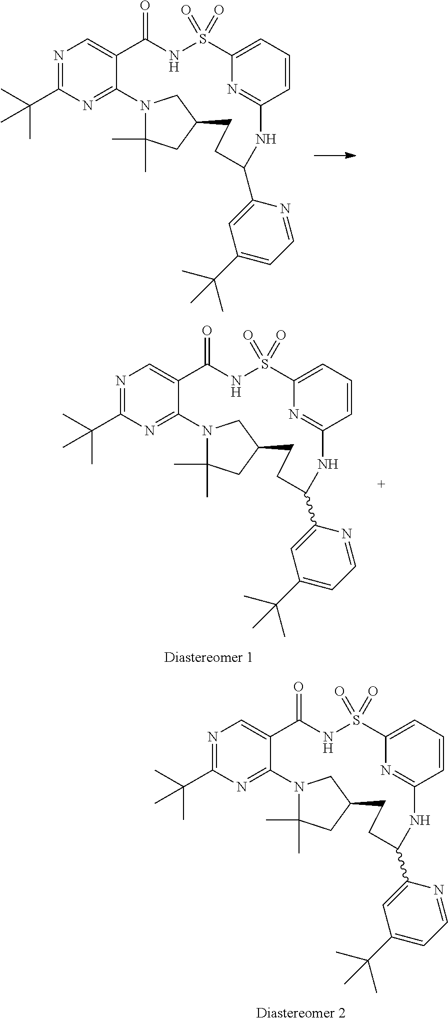

The two reaction mixtures from step 3 were combined and purified by reverse phase chromatography on C₁₈ (120 g, eluting 5 to 100% acetonitrile in water+0.1% HCOOH) and freeze-dried to give (13S)-22-(tert-butyl)-8-(4-(tert-butyl)pyridin-2-yl)-15,15-dimethyl-5-thia-4,7-diaza-2(4,5)-pyrimidina-6(2,6)-pyridina-1(1,3)-pyrrolidinacyclodecaphan-3-one 5,5-dioxide (195 mg, 52%) as a beige solid. The mixture of diastereoisomers was separated by preparative HPLC on Waters XSelect CSH column (75×30 mm, 5 um) using water (10 mM NH₄HCO₂) in MeCN (100% for 3 min, then a linear gradient to 40% MeCN over 1 min, then a linear gradient to 60% MeCN for 11 min, then a linear gradient to 95% MeCN during 2 min, hold for 2 min) and freeze-dried to give two isomers:

Diastereomer 1: (13S)-22-(tert-butyl)-8-(4-(tert-butyl)pyridin-2-yl)-15,15-dimethyl-5-thia-4,7-diaza-2(4,5)-pyrimidina-6(2,6)-pyridina-1(1,3)-pyrrolidinacyclodecaphan-3-one 5,5-dioxide (isomer 1, first eluted product) (62 mg, 21%) as a white fluffy solid. ¹H NMR (400 MHz, DMSO-d₆) δ 8.39 (d, J=5.1 Hz, 1H), 8.25 (s, 1H), 8.13 (s, 1H), 7.52 (t, J=7.8 Hz, 1H), 7.49 (d, J=1.7 Hz, 1H), 7.20 (dd, J=5.3, 1.6 Hz, 1H), 7.11-7.02 (m, 2H), 6.76 (d, J=8.3 Hz, 1H), 5.39-5.29 (m, 1H), 3.61-3.53 (m, 1H), 2.94 (t, J=10.9 Hz, 1H), 2.29-2.15 (m, 1H), 2.01-1.91 (m, 2H), 1.88 (dd, J=11.9, 5.3 Hz, 1H), 1.73-1.44 (m, 9H), 1.32 (s, 9H), 1.25 (s, 9H), ESI-MS m/z calc. 605.3148, found 606.3 (M+1)⁺; Retention time: 2.64 minutes (LC method F).

Diastereomer 2: (13S)-22-(tert-butyl)-8-(4-(tert-butyl)pyridin-2-yl)-15,15-dimethyl-5-thia-4,7-diaza-2(4,5)-pyrimidina-6(2,6)-pyridina-1(1,3)-pyrrolidinacyclodecaphan-3-one 5,5-dioxide (isomer 2, second eluted product) (67 mg, 22%) as a white fluffy solid. ¹H NMR (400 MHz, DMSO-d₆) δ 8.47 (d, J=5.1 Hz, 1H), 8.13 (s, 1H), 8.08 (s, 1H), 7.53-7.45 (m, 2H), 7.27 (dd, J=5.3, 1.8 Hz, 1H), 7.16 (d, J 6.8 Hz, 1H), 6.92 (br. s., 1H), 6.73 (d, J=8.3 Hz, 1H), 5.09-5.01 (m, 1H), 3.77 (br. s., 1H), 3.48-3.37 (m, 1H), 2.23 (br. s., 1H), 2.01 (d, J=6.8 Hz, 2H), 1.85 (dd, J=11.9, 6.0 Hz, 1H), 1.73-1.44 (m, 8H), 1.31 (d, J=4.4 Hz, 18H), 1.22-1.11 (m, 1H). ESI-MS m/z calc. 605.3148, found 606.3 (M+1)⁺; Retention time: 2.56 minutes (LC method F).

### Example 82: Preparation of (188)-11,11,16,16-tetramethyl-21-(pyridin-2-yl)-10-oxa-2λ⁶-thia-3,13,15,22,27-pentaazapentacyclo[21.3.1.115,18.05,14.07,12]octacosa-1(27),5(14),6,12,23,25-hexaene-2,2,4-trione, Compound 213 (diastereomer 1) and Compound 212 (diastereomer 2)

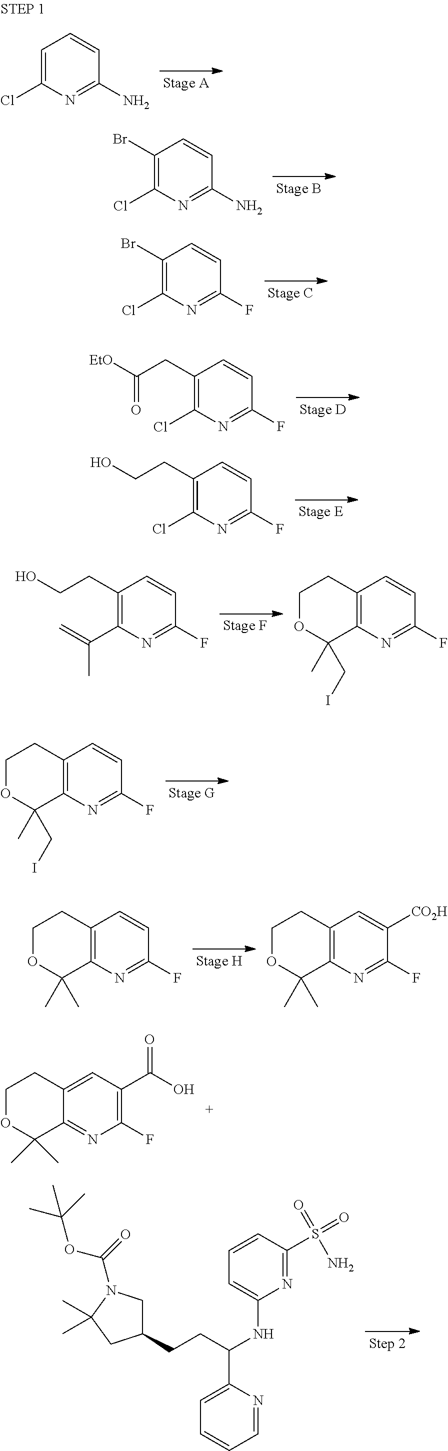

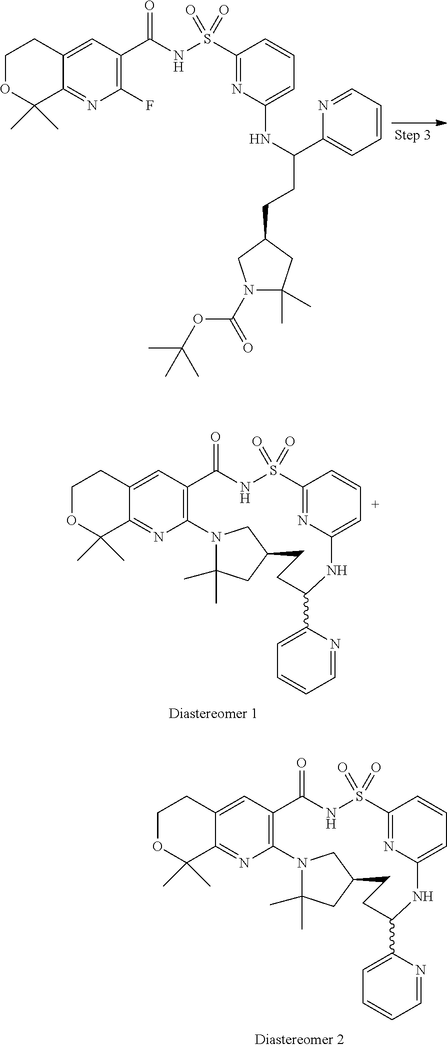 [SMILES: CC(C)(C)OC(=O)N1C[C@@H](CCC(Nc2cccc(S(=O)(=O)NC(=O)c3cc4c(nc3F)C(C)(C)OCC4)n2)c2ccccn2)CC1(C)C ; CC1(C)OCCc2cc3c(nc21)N1C[C@@H](CCC(c2ccccn2)Nc2cccc(n2)S(=O)(=O)NC3=O)CC1(C)C ; CC1(C)OCCc2cc3c(nc21)N1C[C@@H](CCC(c2ccccn2)Nc2cccc(n2)S(=O)(=O)NC3=O)CC1(C)C]

### Step 1: Preparation of 2-fluoro-8,8-dimethyl-6,8-dihydro-5H-pyrano[3,4-b]pyridine-3-carboxylic acid

### Stage A: Synthesis of 5-bromo-6-chloropyridin-2-amine

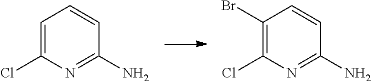 [SMILES: Nc1cccc(Cl)n1 ; Nc1ccc(Br)c(Cl)n1]

A solution of 2-amino-6-chloropyridine (2.00 g, 15.6 mmol) in acetonitrile (20 mL) was treated with N-bromosuccinimide (3.10 g, 17.4 mmol) and stirred overnight at room temperature covered with aluminum foil. The solids were then decanted and washed with another portion of acetonitrile (15 mL). The volatiles were removed under reduced pressure and the residue was purified on a 120-g column, eluting from 0% to 40% EtOAc in heptanes to afford 5-bromo-6-chloropyridin-2-amine (1.75 g, 54% yield) as a white solid. ¹H NMR (300 MHz, DMSO-d₆) δ 6.33 (d, J=8.5 Hz, 1H), 6.58 (s, 2H), 7.61 (d, J=8.5 Hz, 1H). [M+1-1]⁺=207.0.

### Stage B: Synthesis of 3-bromo-2-chloro-6-fluoropyridine

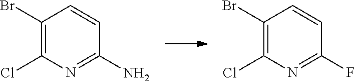 [SMILES: Nc1ccc(Br)c(Cl)n1 ; Fc1ccc(Br)c(Cl)n1]

A solution of 5-bromo-6-chloropyridin-2-amine (3.00 g, 14.5 mmol) in hydrogen fluoride pyridine (72 mL of about 70% HF) was cooled in an ice bath and treated portionwise with solid sodium nitrite (1.20 g, 17.4 mmol). The reaction was then left to gradually warm to room temperature over a period of 3-4 h. Quenched by adding slowly to an aqueous solution of sodium bicarbonate (750 mL), adding solid sodium bicarbonate to ensure that the aqueous solution maintains a pH of about 8. The opaque aqueous layer was transferred to a 2.0-L separatory funnel with water (100 mL) and extracted with EtOAc (3×200 mL). The combined organic layers were washed with water, brine, dried over Na₂SO₄, filtered and concentrated under reduced pressure to give crude 3-bromo-2-chloro-6-fluoropyridine (2.91 g, 96% yield) as an amber oil that still contained pyridine (about 17%). ¹H NMR (300 MHz, CDCl₃) δ 6.81 (ddd, J=8.4, 3.4, 0.9 Hz, 1H), 7.99 (ddd, J=8.4, 7.0, 0.9 Hz, 1H). ¹⁹F NMR (282 MHz, CDCl₃) δ −69.1 (d, J=4.6 Hz, 1 F). This material was used in the following step without further purification.

### Stage C: Synthesis of 2-(2-chloro-6-fluoropyridin-3-yl)acetate

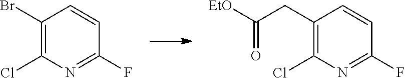 [SMILES: Fc1ccc(Br)c(Cl)n1 ; CCOC(=O)Cc1ccc(F)nc1Cl]

Preparation of the Organozinc Reagent:

To a flame-dried flask was added zinc dust (7.36 g, 113 mmol) and the flask was flame-dried again under nitrogen. Once cooled the zinc was covered with THF (27 mL). Added chlorotrimethylsilane (0.75 mL) and heated the flask close to the boiling point of THF using a heat gun. Once cooled the flask was placed in an oil bath set at 40° C. and treated with a solution of ethyl 2-bromoacetate (9.40 g, 56.3 mmol) in THF (70 mL) over a period of one hour. Stirring was continued for another 30 minutes in the oil bath, then the flask was removed and stirring was stopped to allow the remaining zinc to settle at the bottom of the flask, leaving a clear yellow solution with a concentration of about 0.5 to 0.6 M in organozinc.

To another flame-dried flask was added 3-bromo-2-chloro-6-fluoropyridine (1.50 g, 7.13 mmol) and THF (20 mL). The solution was bubbled with nitrogen gas for about 5 minutes before adding 4,5-bis(diphenylphosphino)-9,9-dimethylxanthene (Xantphos, 206 mg, 0.356 mmol) followed by bis(dibenzylideneacetone)palladium(0) (205 mg, 0.356 mmol). The solution of organozinc reagent previously prepared (43 mL of a 0.5 M solution in THF, 21.5 mmol) was then added, the flask was sealed and heated in an oil bath at 70° C. for 19 h. Note: another reaction was setup in parallel, using 3-bromo-2-chloro-6-fluoropyridine (1.365 g, 6.49 mmol) and following a similar procedure to the one just described.

Both crude reactions were quenched by slowly adding 5% aqueous sodium bicarbonate (about 50 mL) and the mixtures were transferred to a 1.0-L separatory funnel with more 5% aqueous sodium bicarbonate (about 400 mL). The aqueous layer was then extracted with EtOAc (3×200 mL). The combined organic layers were washed with water (100 mL), brine, dried over sodium sulfate, filtered and concentrated under reduced pressure. The residue was purified by silica gel chromatography on a 120-g column, eluting from 0% to 20% EtOAc in heptanes, to afford 2-(2-chloro-6-fluoropyridin-3-yl)acetate (2.225 g, 75% yield) as a yellow oil. ¹H NMR (300 MHz, CDCl₃) δ 1.27 (t, J=7.1 Hz, 1H), 3.75 (s, 2H), 4.19 (q, J=7.1 Hz, 2H), 6.88 (dd, J=8.1, 3.1 Hz, 1H), 7.75 (t, J=7.9, 1H). [M+14]⁺=218.1.

### Stage D: Synthesis of 2-(2-chloro-6-fluoropyridin-3-yl)ethanol

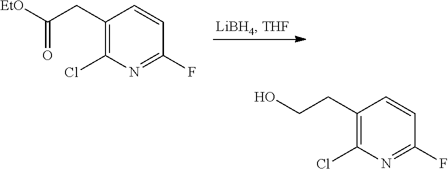 [SMILES: CCOC(=O)Cc1ccc(F)nc1Cl ; OCCc1ccc(F)nc1Cl]

To a solution of ethyl 2-(2-chloro-6-fluoropyridin-3-yl)acetate (2.225 g, 10.23 mmol) in tetrahydrofuran (17 mL) and methanol (1.7 mL) was added lithium borohydride (370 mg, 15.3 mmol) in several portions at 0° C. The mixture was then warmed to room temperature and stirred overnight under nitrogen atmosphere. After completion the mixture was cooled to 0° C. and the pH of the mixture was adjusted to around 7 by addition of saturated aqueous ammonium chloride. The aqueous layer was extracted with ethyl acetate (2×50 mL) and the combined organic layers were dried over sodium sulfate, filtered, and concentrated under pressure. After further drying under high vacuum, pure 2-(2-chloro-6-fluoropyridin-3-yl)ethanol was obtained (1.64 g, 91% yield) as a yellow oil. ¹H NMR (300 MHz, CDCl₃) δ 2.98 (t, J=6.4 Hz, 2H), 3.92 (t, J=6.4 Hz, 2H), 6.85 (dd, J=8.0, 3.2 Hz, 1H), 7.75 (t, J=8.0 Hz, 1H). ¹⁹F NMR (282 MHz, CDCl₃) δ −69.5 (d, J=5.4 Hz, 1 F). [M+14]⁺=176.1.

### Stage E: Synthesis of 2-(6-fluoro-2-(prop-1-en-2-yl)pyridin-3-yl)ethanol

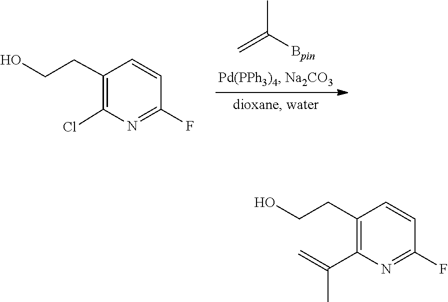

A round bottom flask was charged with 2-(2-chloro-6-fluoropyridin-3-yl)ethanol (1.64 g, 9.34 mmol), isopropenylboronic acid pinacol ester (1.93 mL, 10.3 mmol), sodium carbonate (3.0 g, 28 mmol), tetrakis(triphenylphosphine)palladium (1.08 g, 0.934 mmol), dioxane (47 mL) and water (18 mL). The mixture was heated at 100° C. for 16 h under nitrogen atmosphere. Once cooled the reaction mixture was diluted with ethyl acetate (50 mL), transferred to a separatory funnel and washed with water (20 mL) and brine (20 mL). The organic layer was dried over sodium sulfate, filtered and concentrated under reduced pressure. The residue was purified by silica-gel column chromatography, eluting from 0% to 60% ethyl acetate in heptanes to afford 2-(6-fluoro-2-(prop-1-en-2-yl)pyridin-3-yl)ethanol (1.57 g, 93% yield) as a yellow oil. ¹H NMR (300 MHz, CDCl₃) δ 2.09-2.12 (m, 3H), 2.96 (t, J=6.7 Hz, 2H), 3.82 (t, J=6.7 Hz, 2H), 5.00-5.05 (m, 1H), 5.34-5.38 (m, 1H), 6.78 (dd, J=8.3, 3.4 Hz, 1H), 7.71 (t, J=8.2 Hz, 1H). ¹⁹F NMR (282 MHz, CDCl₃) δ −70.9 (d, J=5.8 Hz, 1 F). [M+H]⁺=182.1.

### Stage F. Synthesis of 2-fluoro-8-(iodomethyl)-8-methyl-6,8-dihydro-5H-pyrano[3,4-b]pyridine

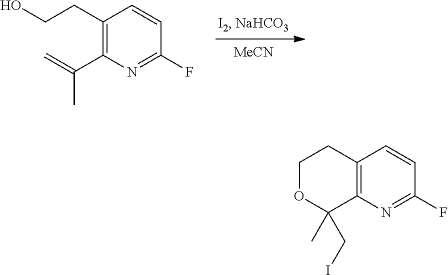

To a stirred solution of 2-(6-fluoro-2-(prop-1-en-2-yl)pyridin-3-yl)ethanol (1.57 g, 8.66 mmol) in acetonitrile (66 mL) containing sodium bicarbonate (2.43 g, 28.9 mmol) at 0° C. under nitrogen atmosphere was added iodine (7.32 g, 28.9 mmol) in several portions and the mixture was stirred at room temperature overnight. After completion, 10% sodium thiosulfate (50 mL) was added and the aqueous layer was extracted with ethyl acetate (50 mL). The organic layer was then washed with brine (30 mL), dried over sodium sulfate, filtered and concentrated under pressure. The residue was purified by silica-gel column chromatography, eluting from 0% to 20% ethyl acetate in heptanes to afford 2-fluoro-8-(iodomethyl)-8-methyl-6,8-dihydro-5H-pyrano[3,4-b]pyridine (1.81 g, 68% yield) as a yellow oil. ¹H NMR (300 MHz, CDCl₃) δ 1.65 (s, 3H), 2.62-2.75 (m, 1H), 3.93-4.06 (m, 1H), 3.58 (d, J=10.3 Hz, 1H), 3.78 (d, J=10.3 Hz, 1H), 3.85-3.94 (m, 1H), 3.98-4.07 (m, 1H), 6.77 (dd, J=8.2, 2.9 Hz, 1H), 7.51 (t, J=8.2 Hz, 1H). ¹⁹F NMR (282 MHz, CDCl₃) δ −70.0 (d, J=6.2 Hz, 1 F). [M+H]⁺=308.0.

### Stage G. Synthesis of 2-fluoro-8,8-dimethyl-6,8-dihydro-5H-pyrano[3,4-b]pyridine

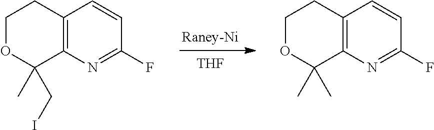

To a stirred solution of 2-fluoro-8-(iodomethyl)-8-methyl-6,8-dihydro-5H-pyrano[3,4-b]pyridine (1.81 g, 5.89 mmol) in tetrahydrofuran (42 mL) was added Raney-Nickel (8 g) and the mixture was stirred at room temperature for 2 days (the flask was sealed with a plastic septum after purging with nitrogen gas). Additional Raney-Nickel (8 g) was added and stirred for an additional day. After completion, the mixture was filtered over Celite and washed with ethyl acetate. The solvent was removed under reduced pressure and the mixture was diluted with ethyl acetate (50 mL). The organic layer was washed with water (20 mL), brine (20 mL), dried over sodium sulfate, filtered, and concentrated under reduced pressure to afford crude 2-fluoro-8,8-dimethyl-6,8-dihydro-5H-pyrano[3,4-b]pyridine (900 mg, 84% yield) as a yellow oil that was used directly in the next step without further purification. [M+4]⁺=182.1. Caution: the compound appears to be volatile and should not be kept under high vacuum for extended periods of time.

### Stage H. Synthesis of 2-Fluoro-8,8-dimethyl-6,8-dihydro-5H-pyrano[3,4-b]pyridine-3-carboxylic acid

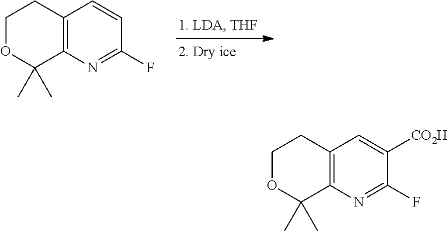 [SMILES: CC1(C)OCCc2ccc(F)nc21 ; CC1(C)OCCc2cc(C(=O)O)c(F)nc21]

To a solution of diisopropylamine (0.78 mL, 5.41 mmol) in tetrahydrofuran (11 mL) at −78° C. under nitrogen atmosphere was added n-butyllithium (2.16 mL of a 2.5 M solution in hexanes, 5.41 mmol) over 10 minutes such that the internal temperature was maintained below −60° C. The mixture was then stirred for 15 minutes at 0° C. before cooling back to −78° C. To this mixture was added a solution of 2-fluoro-8,8-dimethyl-6,8-dihydro-5H-pyrano[3,4-b]pyridine (1.07 g, 5.41 mmol) in tetrahydrofuran (3.0 mL) over 10 minutes, maintaining the internal temperature at −65° C. The reaction was stirred at −78° C. for 4 h then one pellet of dry ice (dried by blowing with nitrogen gas) was added. The mixture was stirred for one hour then gradually warmed up to room temperature overnight (Note: since a lot of carbon dioxide gas evolved upon warming up to room temperature, the reaction flask was capped with a septum and an empty balloon on top). After completion, the reaction was diluted with water (10 mL) and extracted with methyl tert-butyl methyl diethyl ether (2×10 mL). The aqueous layer was acidified with 1.0 N HCl to a pH of 4-5 and extracted with ethyl acetate (2×20 mL). The combined organic layers were dried over sodium sulfate, filtered, and concentrated under reduced pressure. The product was purified by silica gel column chromatography, eluting with 100% ethyl acetate containing 1% formic acid. The fractions collected were concentrated under pressure and the residue was re-dissolved in ethyl acetate (20 mL). The solution was then extracted with saturated sodium bicarbonate (20 mL). The aqueous layer was collected, acidified to a pH of about 4 with 3.0 N HCl and extracted again with ethyl acetate (20 mL). The solvent was removed under reduced pressure to afford 2-fluoro-8,8-dimethyl-6,8-dihydro-5H-pyrano[3,4-b]pyridine-3-carboxylic acid (280 mg, 23% yield) as an off-white solid. ¹H NMR (300 MHz, CDCl₃) δ 1.56 (s, 6H), 2.90 (t, J=5.4 Hz, 2H), 3.98 (t, J=5.6 Hz, 2H), 8.16 (d, J=9.0 Hz, 1H). ¹⁹F NMR (282 MHz, CDCl₃) δ −64.7 (d, J 7.6 Hz, 1 F). [M+1-1]⁺=226.1.

### Step 2: tert-Butyl (4S)-4-[3-[[6-[(2-fluoro-8,8-dimethyl-5,6-dihydropyrano[3,4-b]pyridine-3-carbonyl)sulfamoyl]-2-pyridyl]amino]-3-(2-pyridyl)propyl]-2,2-dimethyl-pyrrolidine-1-carboxylate

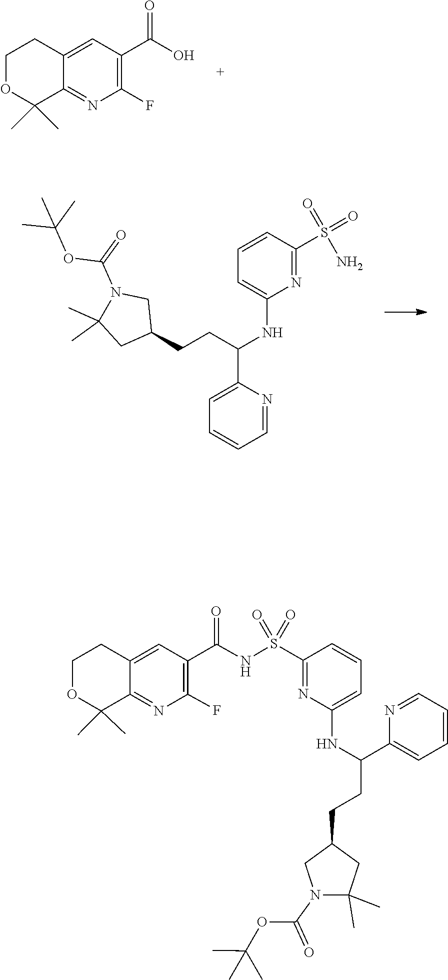 [SMILES: CC1(C)OCCc2cc(C(=O)O)c(F)nc21 ; CC(C)(C)OC(=O)N1C[C@@H](CCC(Nc2cccc(S(N)(=O)=O)n2)c2ccccn2)CC1(C)C ; CC(C)(C)OC(=O)N1C[C@@H](CCC(Nc2cccc(S(=O)(=O)NC(=O)c3cc4c(nc3F)C(C)(C)OCC4)n2)c2ccccn2)CC1(C)C]

To a solution of 2-fluoro-8,8-dimethyl-5,6-dihydropyrano[3,4-b]pyridine-3-carboxylic acid (195 mg, 0.8658 mmol) in THF (5 mL) was added CDI (145 mg, 0.8942 mmol) and the mixture was stirred at rt for 20 h. Then tert-butyl (4S)-2,2-dimethyl-4-[3-(2-pyridyl)-3-[(6-sulfamoyl-2-pyridyl)amino]propyl]pyrrolidine-1-carboxylate (235 mg, 0.4800 mmol) was added followed by DBU (350 μL, 2.340 mmol) and the resulting mixture was stirred for 18 h at rt. The reaction were diluted with ethyl acetate and washed with a saturated aqueous sodium bicarbonate solution. The organic layer was further washed with 10% citric acid solution followed by brine. The organics were separated, dried over sodium sulfate, and evaporated. The crude material was then purified by reverse-phase preparative chromatography utilizing a C₁₈ column and a 1-99% gradient over 15 min of acetonitrile-Water (+5 mM HCl) to afford a diastereomeric pair of tert-butyl (4S)-4-[3-[[6-[(2-fluoro-8,8-dimethyl-5,6-dihydropyrano[3,4-b]pyridine-3-carbonyl)sulfamoyl]-2-pyridyl]amino]-3-(2-pyridyl)propyl]-2,2-dimethyl-pyrrolidine-1-carboxylate (302 mg, 90%) as a white solid. ESI-MS m/z calc. 696.31055, found 696.2 (M+1)⁺; Retention time: 1.58 minutes (LC method A).

### Step 3: (18S)-11,11,16,16-tetramethyl-21-(pyridin-2-yl)-10-oxa-2λ⁶-thia-3,13,15,22,27-pentaazapentacyclo[21.3.1.115,18.05,14.07,12]octacosa-1(27),5(14),6,12,23,25-hexaene-2,2,4-trione, Compound 213 (diastereomer 1) and (185)-11,11,16,16-tetramethyl-21-(pyridin-2-yl)-10-oxa-2λ⁶-thia-3,13,15,22,27-pentaazapentacyclo[21.3.1.115,18.05,14.07,12]octacosa-1(27),5(14),6,12,23,25-hexaene-2,2,4-trione, Compound 212 (diastereomer 2)

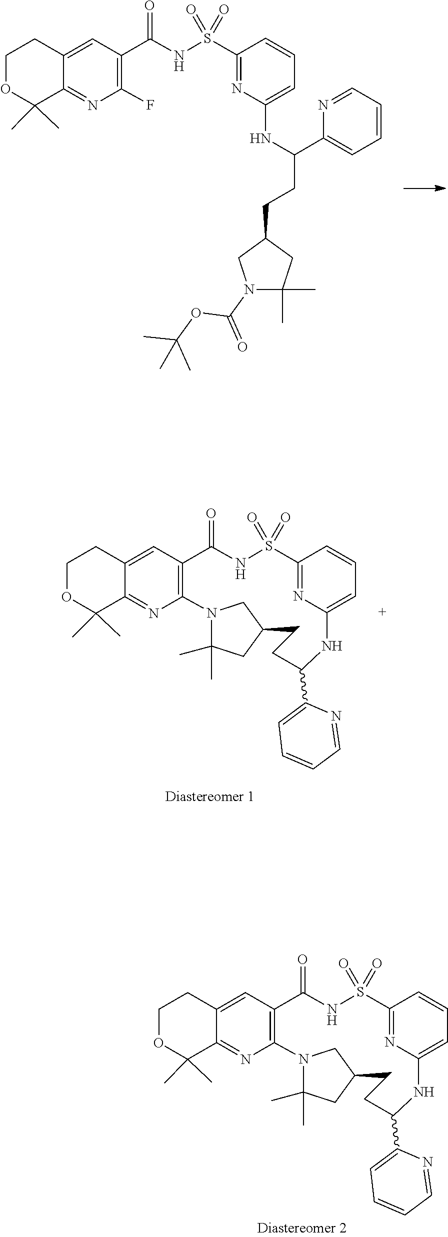 [SMILES: CC(C)(C)OC(=O)N1C[C@@H](CCC(Nc2cccc(S(=O)(=O)NC(=O)c3cc4c(nc3F)C(C)(C)OCC4)n2)c2ccccn2)CC1(C)C ; CC1(C)OCCc2cc3c(nc21)N1C[C@@H](CCC(c2ccccn2)Nc2cccc(n2)S(=O)(=O)NC3=O)CC1(C)C ; CC1(C)OCCc2cc3c(nc21)N1C[C@@H](CCC(c2ccccn2)Nc2cccc(n2)S(=O)(=O)NC3=O)CC1(C)C]

Stage 1: tert-Butyl (4S)-4-[3-[[6-[(2-fluoro-8,8-dimethyl-5,6-dihydropyrano[3,4-b]pyridine-3-carbonyl)sulfamoyl]-2-pyridyl]amino]-3-(2-pyridyl)propyl]-2,2-dimethyl-pyrrolidine-1-carboxylate (302 mg, 0.4334 mmol) was dissolved in DCM (10 mL) and to the mixture was added TFA (1.25 mL, 16.22 mmol) and the reaction was stirred at room temperature. After 1 h, the reactions was complete. The mixture was evaporated to dryness, then diluted with diethyl ether (30 mL×2), and reconcentrated. The material was then placed on a high vacuum pump for 2 h to afford intermediate N-[[6-[[3-[(3S)-5,5-dimethylpyrrolidin-3-yl]-1-(2-pyridyl)propyl]amino]-2-pyridyl]sulfonyl]-2-fluoro-8,8-dimethyl-5,6-dihydropyrano[3,4-b]pyridine-3-carboxamide as a tan residue. ESI-MS m/z calc. 596.2581, found 597.2 (M+1)⁺; Retention time: 0.83 minutes ((LC method A).

Stage 2: The intermediate from Stage 1 and K₂CO₃ (700 mg, 5.065 mmol), 3 Å molecular sieves and NMP (8 mL) was combined in a vial, purged with nitrogen, capped, heated to 150° C. and stirred for 18 h. Cooled to room temperature and the mixture was diluted with ethyl acetate and water. The organic layer was extracted (2×) and was further washed with a 10% citric acid solution followed by brine. The organics were separated, dried over sodium sulfate, evaporated to a light brown oil. The residue was subjected to reverse-phase preparative chromatography utilizing a C₁₈ column and a 1-99% gradient over 15 min of acetonitrile in water (+5 mM HCl) to afford the isomeric products as white solids:

First to elute, diastereomer 1: (18S)-11,11,16,16-Tetramethyl-21-(pyridin-2-yl)-10-oxa-2λ⁶-thia-3,13,15,22,27-pentaazapentacyclo[21.31115,18.05,14.07,12]octacosa-1(27),5(14),6,12,23,25-hexaene-2,2,4-trione (67.3 mg, 52%). ¹H NMR (500 MHz, DMSO-d₆) δ 12.59 (s, 1H), 8.80 (dd, J=5.7, 1.6 Hz, 1H), 8.50 (t, J=8.0 Hz, 1H), 8.20 (d, J 8.3 Hz, 2H), 7.90 (t, J=6.7 Hz, 1H), 7.74 (dd, J=8.5, 7.3 Hz, 1H), 7.36 (d, J=7.3 Hz, 1H), 7.27 (s, 1H), 6.99 (d, J=8.5 Hz, 1H), 5.15 (s, 1H), 3.81 (td, J=5.5, 2.3 Hz, 2H), 3.27 (s, 1H), 2.64 (qt, J=15.8, 5.5 Hz, 3H), 2.29 (s, 1H), 2.13 (d, J=11.1 Hz, 1H), 1.85 (dd, J=11.8, 5.7 Hz, 1H), 1.82-1.72 (m, 1H), 1.49 (d, J=7.2 Hz, 6H), 1.46 (s, 3H), 1.45-1.38 (m, 1H), 1.37 (s, 3H), 1.35-1.20 (m, 1H), 1.20-1.10 (m, 1H). ESI-MS m/z calc. 576.2519, found 577.3 (M+1)⁺; Retention time: 1.26 minutes (LC method A).

Second to elute, diasteseomer 2: (18S)-11,11,16,16-tetramethyl-21-(pyridin-2-yl)-10-oxa-2λ⁶-thia-3,13,15,22,27-pentaazapentacyclo[21.3.1.115,18.05,14.07,12]octacosa-1(27),5(14),6,12,23,25-hexaene-2,2,4-trione (44.1 mg, 34%). ¹H NMR (500 MHz, DMSO-d₆) δ 12.50 (s, 1H), 8.83 (dd, J=5.8, 1.6 Hz, 1H), 8.52 (t, J=7.9 Hz, 1H), 8.18 (d, J=8.5 Hz, 1H), 8.11 (d, J=8.1 Hz, 1H), 7.90 (t, J=6.7 Hz, 1H), 7.74 (dd, J=8.5, 7.3 Hz, 1H), 7.46 (s, 1H), 7.21 (d, J=7.2 Hz, 1H), 7.00 (d, J=8.4 Hz, 1H), 5.57 (t, J=11.7 Hz, 1H), 3.87-3.78 (m, 2H), 3.44 (t, J=8.2 Hz, 1H), 2.74 (dt, J=15.8, 5.4 Hz, 1H), 2.71-2.58 (m, 2H), 2.46 (dd, J=17.5, 5.0 Hz, 1H), 2.12-2.04 (m, 2H), 1.81 (ddd, J=24.1, 13.5, 5.1 Hz, 2H), 1.62 (s, 3H), 1.57 (d, J=12.5 Hz, 1H), 1.53 (s, 3H), 1.49 (d, J=10.3 Hz, 1H), 1.46 (s, 3H), 1.36 (s, 3H). ESI-MS m/z calc. 576.2519, found 577.3 (M+1)⁺; Retention time: 1.39 minutes (LC method A).

### Example 83: Preparation of (18S)-11,11,16,16-tetramethyl-21-(pyridin-2-yl)-2λ⁶-thia-3,13,15,22,27-pentaazapentacyclo[21.3.1.115,18.05,14.07,12]octacosa-1(27),5(14),6,12,23,25-hexaene-2,2,4-trione, Compound 264 (diastereomer 1), and Compound 263 (diastereomer 2)

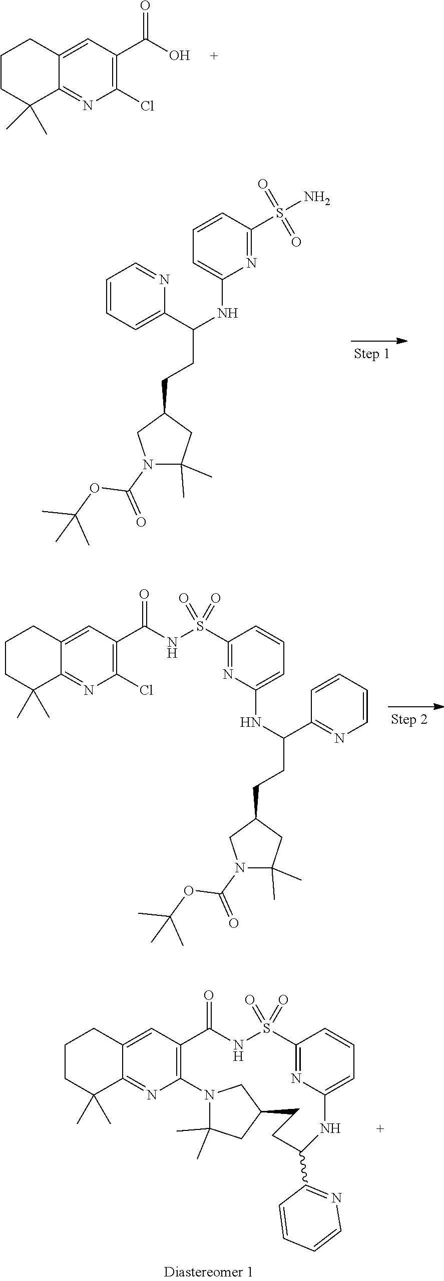 [SMILES: CC1(C)CCCc2cc(C(=O)O)c(Cl)nc21 ; CC(C)(C)OC(=O)N1C[C@@H](CCC(Nc2cccc(S(N)(=O)=O)n2)c2ccccn2)CC1(C)C ; CC(C)(C)OC(=O)N1C[C@@H](CCC(Nc2cccc(S(=O)(=O)NC(=O)c3cc4c(nc3Cl)C(C)(C)CCC4)n2)c2ccccn2)CC1(C)C ; CC1(C)CCCc2cc3c(nc21)N1C[C@@H](CCC(c2ccccn2)Nc2cccc(n2)S(=O)(=O)NC3=O)CC1(C)C]

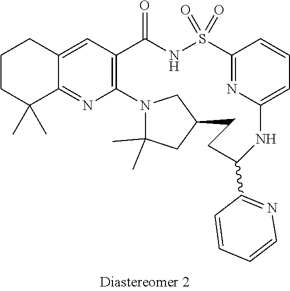 [SMILES: CC1(C)CCCc2cc3c(nc21)N1C[C@@H](CCC(c2ccccn2)Nc2cccc(n2)S(=O)(=O)NC3=O)CC1(C)C]

### Step 1: tert-Butyl (4S)-4-[3-[[6-[(2-chloro-8,8-dimethyl-6,7-dihydro-5H-quinoline-3-carbonyl)sulfamoyl]-2-pyridyl]amino]-3-(2-pyridyl)propyl]-2,2-dimethyl-pyrrolidine-1-carboxylate

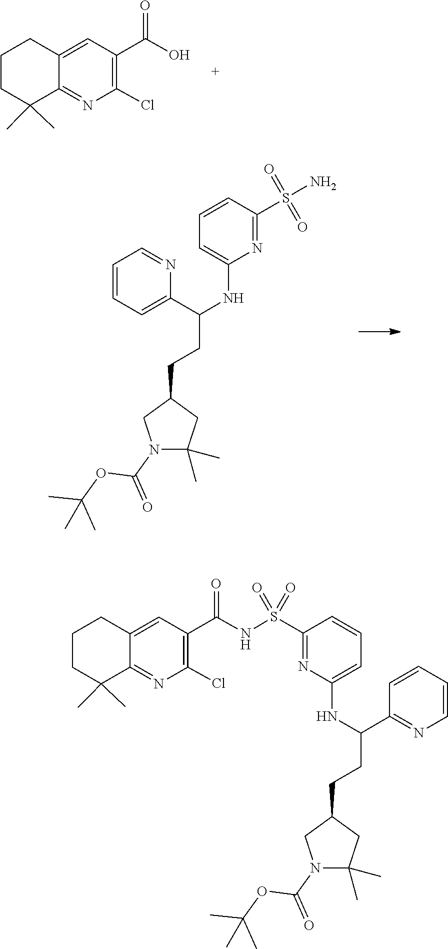 [SMILES: CC1(C)CCCc2cc(C(=O)O)c(Cl)nc21 ; CC(C)(C)OC(=O)N1C[C@@H](CCC(Nc2cccc(S(N)(=O)=O)n2)c2ccccn2)CC1(C)C ; CC(C)(C)OC(=O)N1C[C@@H](CCC(Nc2cccc(S(=O)(=O)NC(=O)c3cc4c(nc3Cl)C(C)(C)CCC4)n2)c2ccccn2)CC1(C)C]

To a solution of 2-chloro-8,8-dimethyl-6,7-dihydro-5H-quinoline-3-carboxylic acid (115 mg, 0.4798 mmol) in THF (4 mL) was added CDI (82 mg, 0.5057 mmol) and the mixture was stirred at rt for 14 h then tert-butyl (4S)-2,2-dimethyl-4-[3-(2-pyridyl)-3-[(6-sulfamoyl-2-pyridyl)amino]propyl]pyrrolidine-1-carboxylate (180 mg, 0.3676 mmol) was added followed by DBU (180 μL, 1.204 mmol) and the resulting mixture was stirred for 16 h at rt. The reaction was diluted with ethyl acetate and washed with a saturated aqueous sodium bicarbonate solution. The organic layer was further washed with 10% citric acid solution followed by brine. The organics were separated, dried over sodium sulfate, evaporated and then the residue was purified by reverse-phase preparative chromatography utilizing a C₁₈ column and a 10-99% gradient over 15 min of acetonitrile in water (+5 mM HCl) to afford tert-butyl (4S)-4-[3-[[6-[(2-chloro-8,8-dimethyl-6,7-dihydro-5H-quinoline-3-carbonyl)sulfamoyl]-2-pyridyl]amino]-3-(2-pyridyl)propyl]-2,2-dimethyl-pyrrolidine-1-carboxylate (153 mg, 59%) as an off-white solid. ESI-MS m/z calc. 710.3017, found 711.2 (M+1)⁺; Retention time: 1.84 minutes (LC method A).

### Step 2: (18S)-11,11,16,16-tetramethyl-21-(pyridin-2-yl)-2λ⁶-thia-3,13,15,22,27-pentaazapentacyclo[21.3.1.115,18.05,14.07,12]octacosa-1(27),5(14),6,12,23,25-hexaene-2,2,4-trione, Compound 264 (diastereomer 1), and (185)-11,11,16,16-tetramethyl-21-(pyridin-2-yl)-2λ⁶-thia-3,13,15,22,27-pentaazapentacyclo[21.3.1.115,18.05,14.07,12]octacosa-1(27),5(14),6,12,23,25-hexaene-2,2,4-trione, Compound 263 (diastereomer 2)

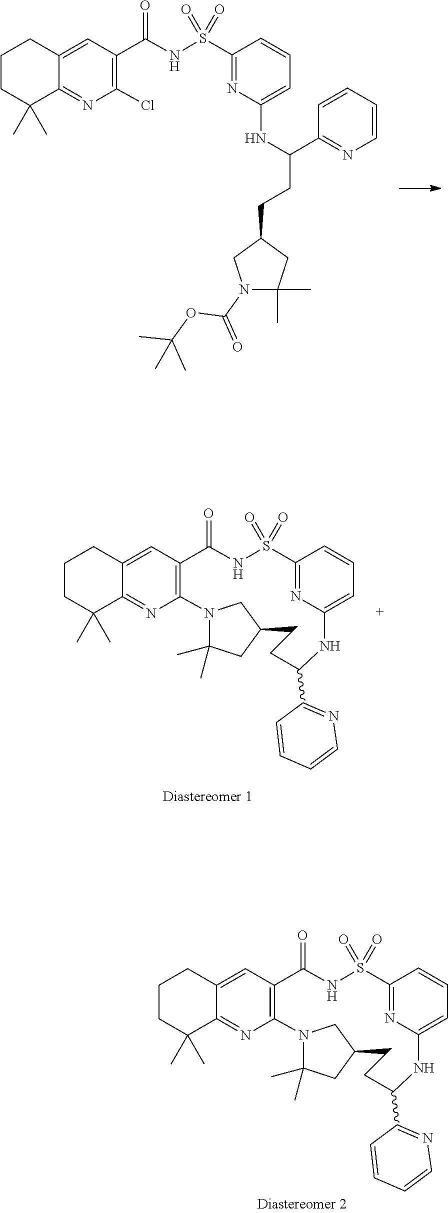 [SMILES: CC(C)(C)OC(=O)N1C[C@@H](CCC(Nc2cccc(S(=O)(=O)NC(=O)c3cc4c(nc3Cl)C(C)(C)CCC4)n2)c2ccccn2)CC1(C)C ; CC1(C)CCCc2cc3c(nc21)N1C[C@@H](CCC(c2ccccn2)Nc2cccc(n2)S(=O)(=O)NC3=O)CC1(C)C ; CC1(C)CCCc2cc3c(nc21)N1C[C@@H](CCC(c2ccccn2)Nc2cccc(n2)S(=O)(=O)NC3=O)CC1(C)C]

Stage 1:

tert-Butyl (4S)-4-[3-[[6-[(2-chloro-8,8-dimethyl-6,7-dihydro-5H-quinoline-3-carbonyl)sulfamoyl]-2-pyridyl]amino]-3-(2-pyridyl)propyl]-2,2-dimethyl-pyrrolidine-1-carboxylate (126 mg, 0.1771 mmol) was dissolved in DCM (3 mL) and to the mixture was added TFA (500 μL, 6.490 mmol) and stirred at room temperature. After 2 h, the reaction was complete. The mixture was evaporated to dryness, then diluted with diethyl ether (30 mL×2), and reconcentrated. The material was then placed on the high vacuum pump for 2 h to afford the intermediate 2-chloro-N-[[6-[[3-[(3S)-5,5-dimethylpyrrolidin-3-yl]-1-(2-pyridyl)propyl]amino]-2-pyridyl]sulfonyl]-8,8-dimethyl-6,7-dihydro-5H-quinoline-3-carboxamide as an off-white solid. ESI-MS m/z calc. 610.24927, found 611.2 (M+1)⁺; Retention time: 1.13 minutes (LC method A).

Stage 2:

Combined material from Step 1 and K₂CO₃ (275 mg, 1.990 mmol), 3 Å molecular sieves and NMP (4 mL) in a vial, purged with nitrogen, capped, heated to 170° C. and stirred for 84 h. Cooled to room temperature and the mixture was diluted with ethyl acetate and water. The organic layer was extracted (2×) and was further washed with 10% citric acid solution followed by brine. The organics were separated, dried over sodium sulfate, evaporated to a light brown oil. This residue was purified by reverse-phase preparative chromatography utilizing a C₁₈ column and a 20-80% gradient over 15 min of acetonitrile in water (+5 mM HCl) to afford two products:

Diastereomer 1, more polar, tan solid: (18S)-11,11,16,16-tetramethyl-21-(pyridin-2-yl)-2λ⁶-thia-3,13,15,22,27-pentanzapentacyclo[21.3.1.115,18.05,14.07,12]octacosa-1(27),5(14),6,12,23,25-hexaene-2,2,4-trione (hydrochloride salt) (14.33 mg, 26%). ¹H NMR (499 MHz, DMSO-d₆) δ 12.38 (s, 1H), 8.72 (d, J=5.4 Hz, 1H), 8.26 (s, 1H), 7.87 (d, J=94.5 Hz, 2H), 7.70 (t, J=7.8 Hz, 2H), 7.31 (d, J=7.1 Hz, 1H), 7.19 (s, 1H), 6.94 (d, J=8.3 Hz, 1H), 5.07 (s, 1H), 3.19 (s, 1H), 2.82 (s, 1H), 2.66-2.56 (m, 2H), 2.56-2.52 (m, 1H), 2.28 (d, J=17.0 Hz, 1H), 2.15 (s, 1H), 1.84 (dd, J=11.8, 5.8 Hz, 1H), 1.77-1.66 (m, 5H), 1.54 (d, J=12.6 Hz, 6H), 1.48 (d, J=11.6 Hz, 1H), 1.27 (s, 3H), 1.22 (d, J=5.2 Hz, 1H), 1.19 (s, 3H). ESI-MS m/z calc. 574.2726, found 575.2 (M+1)⁺; Retention time: 1.51 minutes (LC method A).

Diastereomer 2, less polar, tan solid: (18S)-11,11,16,16-tetramethyl-21-(pyridin-2-yl)-2λ⁶-thia-3,13,15,22,27-pentanzapentacyclo[21.3.1.115,18.05,14.07,12]octacosa-1(27),5(14),6,12,23,25-hexaene-2,2,4-trione (hydrochloride salt) (12.77 mg, 23%). ¹H NMR (499 MHz, DMSO-d₆) δ 12.39 (s, 1H), 8.80-8.64 (m, 1H), 8.27 (s, 1H), 7.90 (d, J=9.0 Hz, 2H), 7.70 (dd, J=8.5, 7.3 Hz, 2H), 7.34 (s, 1H), 7.17 (d, J=7.3 Hz, 1H), 6.94 (d, J=8.4 Hz, 1H), 5.43 (s, 1H), 3.30 (dd, J=9.6, 7.0 Hz, 1H), 2.71-2.62 (m, 2H), 2.59-2.50 (m, 2H), 2.35 (p, J=1.9 Hz, 1H), 2.04-1.93 (m, 2H), 1.82 (dd, J=11.6, 5.2 Hz, 1H), 1.79-1.69 (m, 4H), 1.64 (s, 4H), 1.54 (t, J=12.4 Hz, 1H), 1.48 (s, 3H), 1.31 (s, 3H), 1.16 (s, 3H). ESI-MS m/z calc. 574.2726, found 575.2 (M+1)⁺; Retention time: 1.68 minutes (LC method A).

### Example 84: Preparation of (14S)-8-tert-Butyl-12,12-dimethyl-17-(prop-2-en-1-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 35 (diastereomer 1), and Compound 34 (diastereomer 2)

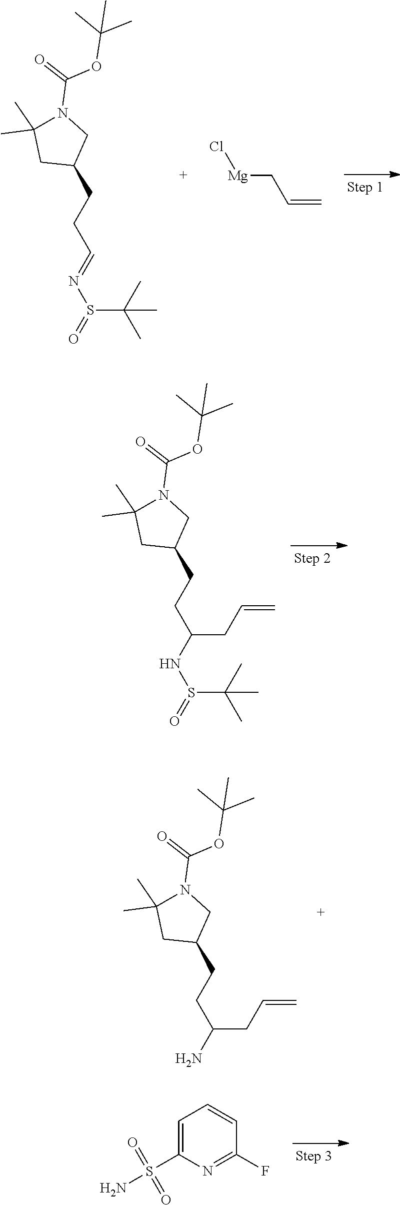

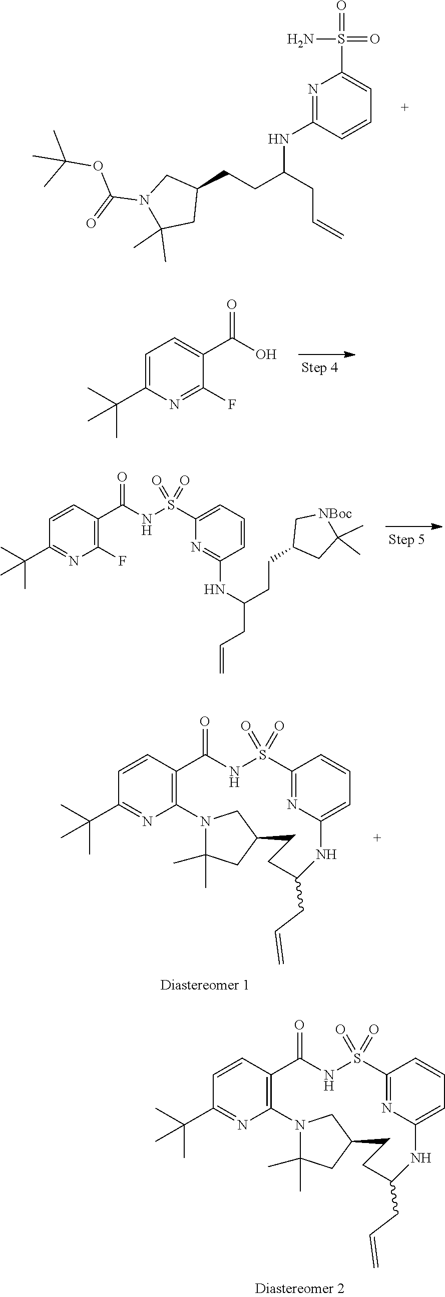

### Step 1: tert-Butyl (4S)-4-[3-(tert-butylsulfinylamino)hex-5-enyl]-2,2-dimethyl-pyrrolidine-1-carboxylate

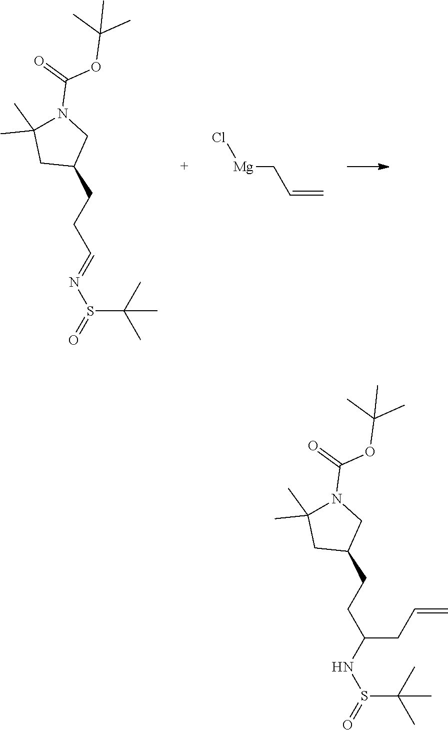

A solution of tert-butyl (4S)-4-[(3E)-3-tert-butylsulfinyliminopropyl]-2,2-dimethyl-pyrrolidine-1-carboxylate (10.5 g, 29.286 mmol) in anhydrous THF (175 mL) was cooled to −70° C. using a dry ice/acetone bath and stirred for 10 minutes. Allyl magnesium chloride (88 mL of 2 M in THF, 176.00 mmol) was then added and the solution was brought up to −40° C. in a dry ice/acetonitrile bath and stirred for 1 hour. Saturated aqueous NH₄Cl (200 mL) was added, then the solution was allowed to warm up to rt. EtOAc (200 mL) and water (100 mL) was added to partition. Aqueous solution was extracted with EtOAc (2×150 mL). The combined organic layers were washed with brine, dried over anhydrous Na₂SO₄, filtered, and concentrated under reduced pressure. Crude product obtained was purified by flash chromatography (load with SiO2) (220 g SiO2, eluting from 0 to 50% ethyl acetate in hexane) to afford tert-butyl (4S)-4-[3-(tert-butylsulfinylamino)hex-5-enyl]-2,2-dimethyl-pyrrolidine-1-carboxylate (11.56 g, 99%) as a light gold gel. ESI-MS m/z calc. 400.276, found 401.3 (M+1)⁺; Retention time: 6.36 minutes (LC method C).

### Step 2: tert-Butyl (4S)-4-(3-aminohex-5-enyl)-2,2-dimethyl-pyrrolidine-1-carboxylate

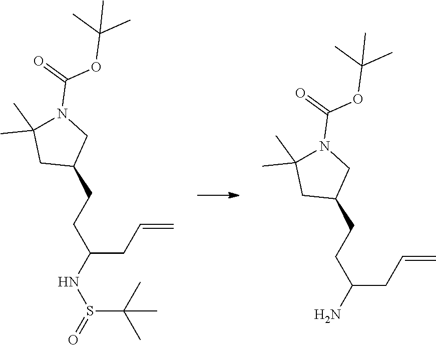 [SMILES: C=CCC(CC[C@@H]1CN(C(=O)OC(C)(C)C)C(C)(C)C1)NS(=O)C(C)(C)C ; C=CCC(N)CC[C@@H]1CN(C(=O)OC(C)(C)C)C(C)(C)C1]

tert-Butyl (4S)-4-[3-(tert-butylsulfinylamino)hex-5-enyl]-2,2-dimethyl-pyrrolidine-1-carboxylate (11.497 g, 28.698 mmol) was dissolved in THF (110 mL) and Water (18 mL). Molecular iodine (2.2 g, 0.4462 mL, 8.6679 mmol) was then added and the solution was stirred at 52° C. for 3 h. The solution was cooled to rt then partitioned with EtOAc (250 mL) and Na₂S₂O₃ (75 g) in saturated aqueous sodium bicarbonate (250 mL). The layers were separated and the aqueous layer was extracted with EtOAc (2×150 mL). The organic layers were combined, washed with brine, dried over anhydrous sodium sulfate, and concentrated under reduced pressure to yield crude tert-butyl (4S)-4-(3-aminohex-5-enyl)-2,2-dimethyl-pyrrolidine-1-carboxylate (10.077 g, 116%) as a gold gel. ESI-MS m/z calc. 296.2464, found 297.4 (M+1)⁺; Retention time: 2.5 minutes (LC method B).

### Step 3: tert-Butyl (4S)-2,2-dimethyl-4-[3-[(6-sulfamoyl-2-pyridyl)amino]hex-5-enyl]pyrrolidine-1-carboxylate

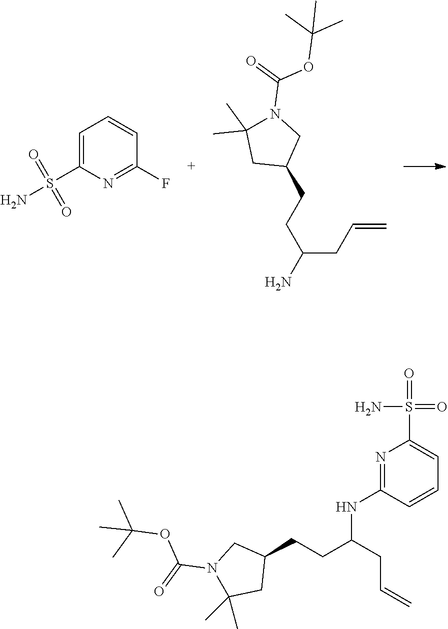 [SMILES: NS(=O)(=O)c1cccc(F)n1 ; C=CCC(N)CC[C@@H]1CN(C(=O)OC(C)(C)C)C(C)(C)C1 ; C=CCC(CC[C@@H]1CN(C(=O)OC(C)(C)C)C(C)(C)C1)Nc1cccc(S(N)(=O)=O)n1]

To a mixture of crude tert-butyl (4S)-4-(3-aminohex-5-enyl)-2,2-dimethyl-pyrrolidine-1-carboxylate (10.77 g, 36.330 mmol) and 6-fluoropyridine-2-sulfonamide (10.880 g, 61.761 mmol) dissolved in anhydrous DMSO (32 mL), was added DIEA (21 mL, 120.56 mmol). The solution was stirred at 115° C. for 68 h then cooled to rt and then diluted with water (300 mL) and EtOAc (250 mL). The layers were separated and the aqueous layer was extracted with EtOAc (2×150 mL). The organic layers were combined and washed with brine (2×150 mL), dried over anhydrous Na₂SO₄, and concentrated under reduced pressure. The crude product was purified by flash chromatography (load with SiO2) (330 g SiO2, eluting from 0 to 50% acetone in hexane) and then purified again by flash chromatography (load with SiO2) (330 g SiO2, eluting from 0-50% Acetone in hexane) to afford tert-butyl (4S)-2,2-dimethyl-4-[3-[(6-sulfamoyl-2-pyridyl)amino]hex-5-enyl]pyrrolidine-1-carboxylate (7.945 g, 45%) as an off white solid. ¹H NMR (500 MHz, DMSO-d₆) δ 7.53-7.46 (m, 1H), 7.06 (s, 2H), 6.97-6.91 (m, 1H), 6.83 (d, J=8.2 Hz, 1H), 6.62 (d, J=8.4 Hz, 1H), 5.89-5.77 (m, 1H), 5.10-4.97 (m, 2H), 4.07 (d, J=8.0 Hz, 1H), 3.58-3.46 (m, 1H), 2.81-2.71 (m, 1H), 2.26 (dtd, J=20.7, 13.8, 6.6 Hz, 2H), 2.05 (d, J 6.1 Hz, 1H), 1.95-1.77 (m, 1H), 1.59-1.42 (m, 2H), 1.40 (s, 6H), 1.38-1.31 (m, 9H), 1.23 (s, 3H). ESI-MS m/z calc. 452.2457, found 453.1 (M+1)⁺; Retention time: 2.81 minutes (LC method H).

### Step 4: tert-Butyl (4S)-4-[3-[[6-[(6-tert-butyl-2-fluoro-pyridine-3-carbonyl)sulfamoyl]-2-pyridyl]amino]hex-5-enyl]-2,2-dimethyl-pyrrolidine-1-carboxylate

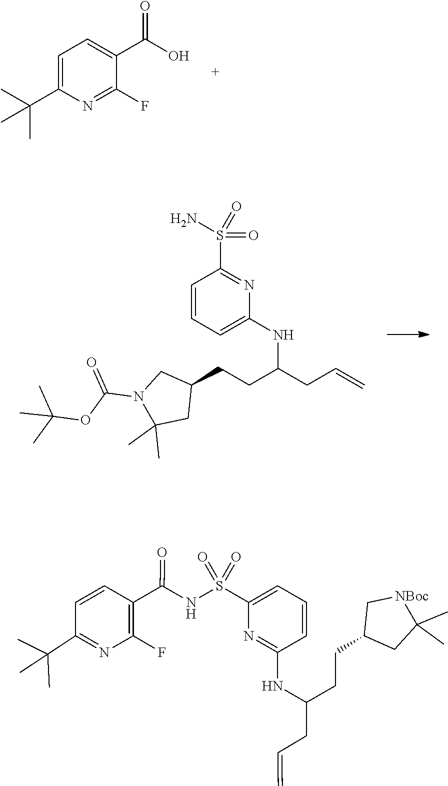

In a 250-mL round-bottomed flask, 6-tert-butyl-2-fluoro-pyridine-3-carboxylic acid (4.0091 g, 20.33 mmol) was dissolved in THF (100 mL), to which CDI (3.5 g, 21.59 mmol) was added. The resulting mixture was stirred at room temperature for 14 h. After this time, tert-butyl (4S)-2,2-dimethyl-4-[3-[(6-sulfamoyl-2-pyridyl)amino]hex-5-enyl]pyrrolidine-1-carboxylate (5 g, 10.61 mmol) and DBU (6 mL, 40.12 mmol) were added, and the resulting mixture was stirred at room temperature for 24 h. After this time, the mixture was concentrated in vacuo. Then, it was diluted with EtOAc (300 mL). This mixture was then washed with saturated aqueous sodium bicarbonate solution (200 mL), aqueous HCl solution (0.1 N, 200 mL) and saturated aqueous NaCl solution (300 mL), then dried over Na₂SO₄, filtered, and evaporated in vacuo. Purification by silica gel chromatography (220 g of silica) using a gradient eluent of 0 to 100% EtOAc in hexanes gave a yellow foam, tert-butyl(4S)-4-[3-[[6-[(6-tert-butyl-2-fluoro-pyridine-3-carbonyl)sulfamoyl]-2-pyridyl]amino]hex-5-enyl]-2,2-dimethyl-pyrrolidine-1-carboxylate (6.56 g, 98%) ESI-MS m/z calc. 631.3204, found 632.4 (M+1)⁺; Retention time: 2.27 minutes and 2.30 minutes (LC method A).

### Step 5: (14S)-8-tert-Butyl-12,12-dimethyl-17-(prop-2-en-1-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 35 (diastereomer 1) and (14S)-8-tert-butyl-12,12-dimethyl-17-(prop-2-en-1-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 34 (diastereomer 2)

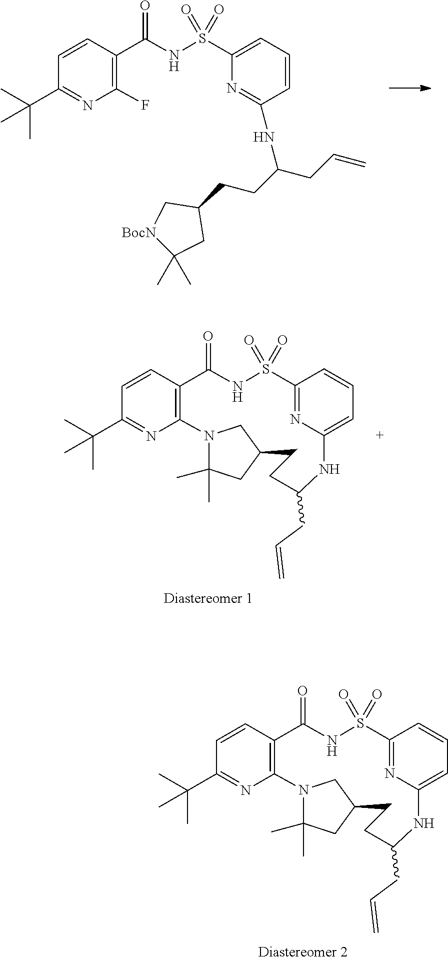

Stage 1: In a 20-mL vial, tert-butyl (4S)-4-[3-[[6-[(6-tert-butyl-2-fluoro-pyridine-3-carbonyl)sulfamoyl]-2-pyridyl]amino]hex-5-enyl]-2,2-dimethyl-pyrrolidine-1-carboxylate (2300 mg, 3.640 mmol) was dissolved in dichloromethane (30 mL). TFA (5 mL, 64.90 mmol) was added, and the resulting solution was allowed to stand at room temperature for 12 h. The mixture was then evaporated in vacuo, diluted with dioxane, and evaporated in vacuo again. This gave a yellow oil, 4100 mg (>100% yield).

Stage 2: In a 100-mL round bottom, the crude product from Step 1 was dissolved in NMP (35 mL), to which K₂CO₃ (8 g, 57.88 mmol) was added. The resulting mixture was flushed with nitrogen, then stirred at 150° C. for 17 h. After cooling to room temperature, the resulting mixture was poured into aqueous HCl solution (1 N; 80 mL), then extracted with EtOAc (2×80 mL). The combined organic extracts was washed with H₂O (80 mL) and saturated aqueous NaCl solution (80 mL), then dried over Na₂SO₄, filtered, and evaporated in vacuo. Purification by silica gel chromatography (220 g of silica) using a gradient eluent of 1 to 30% EtOAc in hexanes gave reasonable separation of diastereomers. Collected pure fractions and pooled mix fractions for potential future repurification. The less polar peak on silica was assigned as peak 1 and the more polar peak as peak 2.

Diastereomer 1, “Peak 1”: (14S)-8-tert-Butyl-12,12-dimethyl-17-(prop-2-en-1-yl)-2λ⁶-thia-3,9,11,18,23-pentaazatetracyclo[17.3.1.111,14.05,10]tetracosa-1(23),5(10),6,8,19,21-hexaene-2,2,4-trione (522 mg, 56%). ¹H NMR (400 MHz, DMSO-d₆) δ 12.40 (s, 1H), 7.57 (dd, J=8.5, 7.3 Hz, 1H), 7.40 (d, J=7.9 Hz, 1H), 7.20 (dd, J=11.8, 7.2 Hz, 2H), 6.72 (d, J=8.5 Hz, 1H), 6.61 (d, J=7.9 Hz, 1H), 5.90-5.75 (m, 1H), 5.04-4.92 (m, 2H), 3.42 (s, 1H), 3.17 (s, 1H), 2.86 (s, 1H), 2.33-2.08 (m, 3H), 1.82 (dd, J=11.8, 5.8 Hz, 1H), 1.52 (m, 9H), 1.26 (m, 10H), 1.19-1.08 (m, 1H). ESI-MS m/z calc. 511.26172, found 512.3 (M+1)⁺; Retention time: 2.14 minutes (LC method A).

Diastereomer 2, “Peak 2”: (14S)-8-tert-Butyl-12,12-dimethyl-17-(prop-2-en-1-yl)-2λ⁶-thia-3,9,11,18,23-pentaazatetracyclo[17.3.1.111,14.05,10]tetracosa-1(23),5(10),6,8,19,21-hexaene-2,2,4-trione (566.2 mg, 61%). ¹H NMR (400 MHz, DMSO-d₆) δ 12.40 (s, 1H), 7.57 (dd, J=8.5, 7.3 Hz, 1H), 7.40 (d, J=7.9 Hz, 1H), 7.20 (dd, J=11.8, 7.2 Hz, 2H), 6.72 (d, J=8.5 Hz, 1H), 6.61 (d, J=7.9 Hz, 1H), 5.90-5.75 (m, 1H), 5.04-4.92 (m, 2H), 3.42 (s, 1H), 3.17 (s, 1H), 2.86 (s, 1H), 2.33-2.08 (m, 3H), 1.82 (dd, J=11.8, 5.8 Hz, 1H), 1.52 (m, 9H), 1.26 (m, 10H), 1.19-1.08 (m, 1H). ESI-MS m/z calc. 511.26172, found 512.3 (M+1)⁺; Retention time: 2.13 minutes (LC method A).

### Example 85: Preparation of (14S)-8-tert-Butyl-17-(2,3-dihydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 33 (enantiomer 1), Compound 32 (enantiomer 2), Compound 30 (enantiomer 3), and Compound 31 (enantiomer 4)

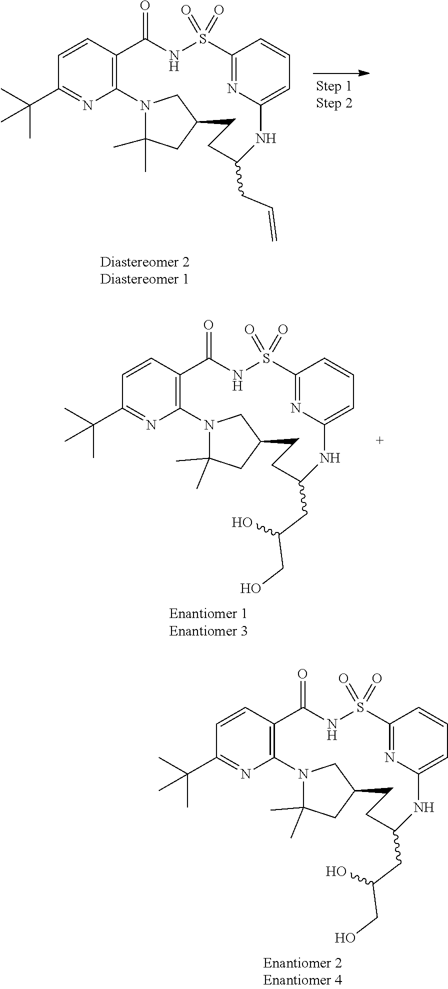 [SMILES: C=CCC1CC[C@@H]2CN(c3nc(C(C)(C)C)ccc3C(=O)NS(=O)(=O)c3cccc(n3)N1)C(C)(C)C2 ; CC(C)(C)c1ccc2c(n1)N1C[C@@H](CCC(CC(O)CO)Nc3cccc(n3)S(=O)(=O)NC2=O)CC1(C)C ; CC(C)(C)c1ccc2c(n1)N1C[C@@H](CCC(CC(O)CO)Nc3cccc(n3)S(=O)(=O)NC2=O)CC1(C)C]

### Step 1: (14S)-8-tert-Butyl-17-(2,3-dihydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 33 (enantiomer 1) and (14S)-8-tert-butyl-17-(2,3-dihydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 32 (enantiomer 2)

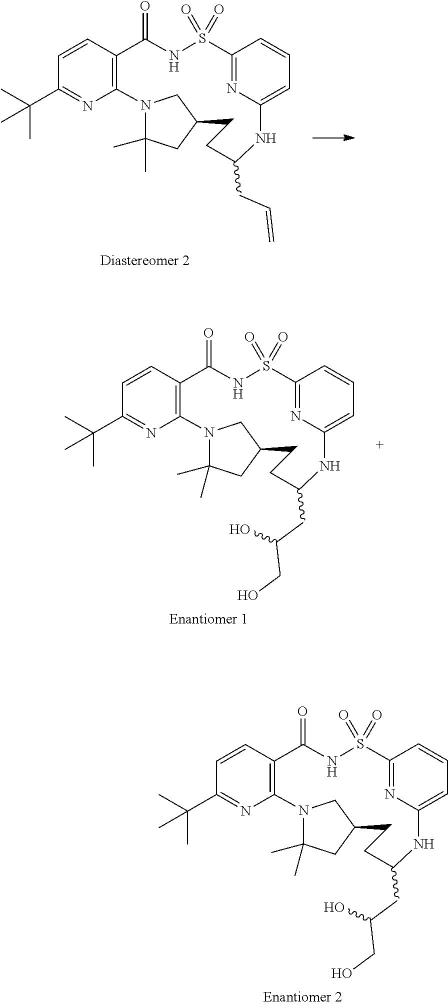 [SMILES: C=CCC1CC[C@@H]2CN(c3nc(C(C)(C)C)ccc3C(=O)NS(=O)(=O)c3cccc(n3)N1)C(C)(C)C2 ; CC(C)(C)c1ccc2c(n1)N1C[C@@H](CCC(CC(O)CO)Nc3cccc(n3)S(=O)(=O)NC2=O)CC1(C)C ; CC(C)(C)c1ccc2c(n1)N1C[C@@H](CCC(CC(O)CO)Nc3cccc(n3)S(=O)(=O)NC2=O)CC1(C)C]

Added 4-methyl-4-oxido-morpholin-4-ium (20 mg, 0.1707 mmol) and potassium osmate(VI) dihydrate (4 mg, 0.01086 mmol) to a 1 dram vial under nitrogen. Added (14S)-8-tert-butyl-12,12-dimethyl-17-(prop-2-en-1-yl)-2λ⁶-thia-3,9,11,18,23-pentaazatetracyclo[17.3.1.111,14.05,10]tetracosa-1(23),5(10),6,8,19,21-hexaene-2,2,4-trione (Compound 34 (diastereomer 2), 50 mg, 0.09772 mmol) in acetone (1 mL) followed by water (100 μL). Stirred overnight (resulted in dark black solution). Quenched with sodium thiosulfate and water. Poured into DCM and separated using a PTFE frit. Back extracted once. Evaporated off the volatiles and took up the crude in DMSO and purified via preparative HPLC (C₁₈ ACN/Water HCl modifier, 15 minutes gradient 0-99% of acetonitrile). Two isomers were isolated after evaporation of the pure fractions:

Peak 1, enantiomer 1: (14S)-8-tert-butyl-17-(2,3-dihydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (1.0 mg, 2%). ESI-MS m/z calc. 545.2672, found 546.2 (M+1)⁺; Retention time: 1.76 minutes. (LC method A).

Peak 2, enantiomer 2: (14S)-8-tert-butyl-17-(2,3-dihydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (2.3 mg, 4%). ESI-MS m/z calc. 545.2672, found 546.27 (M+1)⁺; Retention time: 1.8 minutes (LC method A).

### Step 2: (14S)-8-tert-Butyl-17-(2,3-dihydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 30 (enantiomer 3), and (14S)-8-tert-butyl-17-(2,3-dihydroxypropyl)-12,12-dimethyl-2λ⁶-thia-3,9,11,18,23-pentaazatetracyclo [17.3.1.111,14.05,10]tetracosa-1(23),5(10),6,8,19,21-hexaene-2,2,4-trione, Compound 31 (enantiomer 4)

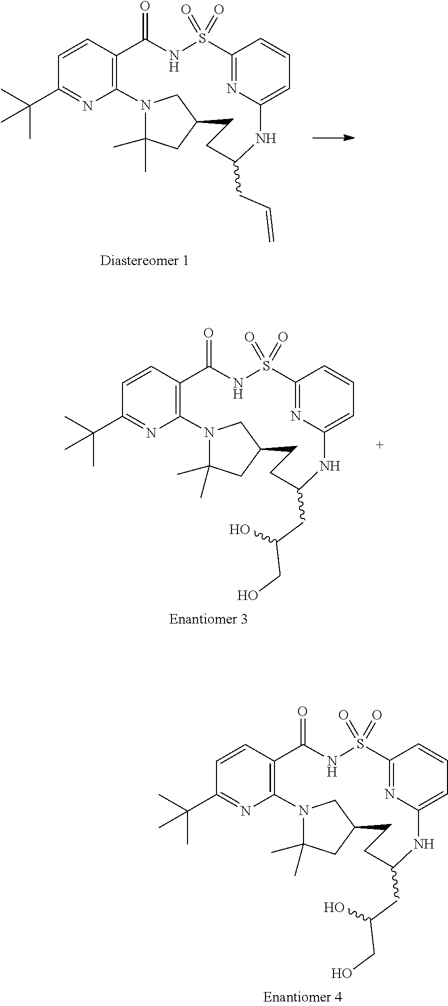 [SMILES: C=CCC1CC[C@@H]2CN(c3nc(C(C)(C)C)ccc3C(=O)NS(=O)(=O)c3cccc(n3)N1)C(C)(C)C2 ; CC(C)(C)c1ccc2c(n1)N1C[C@@H](CCC(CC(O)CO)Nc3cccc(n3)S(=O)(=O)NC2=O)CC1(C)C ; CC(C)(C)c1ccc2c(n1)N1C[C@@H](CCC(CC(O)CO)Nc3cccc(n3)S(=O)(=O)NC2=O)CC1(C)C]

Added 4-methyl-4-oxido-morpholin-4-ium (12 mg, 0.1024 mmol) and potassium osmate(VI) dihydrate (2 mg, 0.005428 mmol) to a 1 dram vial under nitrogen. Added (14S)-8-tert-butyl-12,12-dimethyl-17-(prop-2-en-1-yl)-2λ⁶-thia-3,9,11,18,23-pentaazatetracyclo[17.3.1.111,14.05,10]tetracosa-1(23),5(10),6,8,19,21-hexaene-2,2,4-trione, (Compound 35 (diastereomer 1), 25 mg, 0.04886 mmol) in acetone (0.5 mL) followed by water (125 μL). Stirred overnight. Quenched with sodium thiosulfate and water. Poured into DCM and separated using a PTFE frit. Back extracted once. Evaporated off the volatiles. Took up crude in DMSO and purified via preparative HPLC (C₁₈ ACN/Water HCl modifier, 15 minutes gradient 0-99% of ACN). Two isomers were isolated after evaporation of the pure fractions:

Peak 1 (Lower retention time on UPLC), enantiomer 3: (14S)-8-tert-Butyl-17-(2,3-dihydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (6.8 mg, 26%). ESI-MS m/z calc. 545.2672, found 546.5 (M+1)⁺; Retention time: 1.64 minutes (LC method A).

Peak 2, enantiomer 4: (14S)-8-tert-Butyl-17-(2,3-dihydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (4.9 mg, 18%). ESI-MS m/z calc. 545.2672, found 546.5 (M+1)⁺; Retention time: 1.66 minutes (LC method A).

### Example 86: Preparation of (14S)-8-tert-Butyl-17-[(2E)-4-hydroxy-4-methylpent-2-en-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 24 (diastereomer 1) and Compound 23 (diastereomer 2)

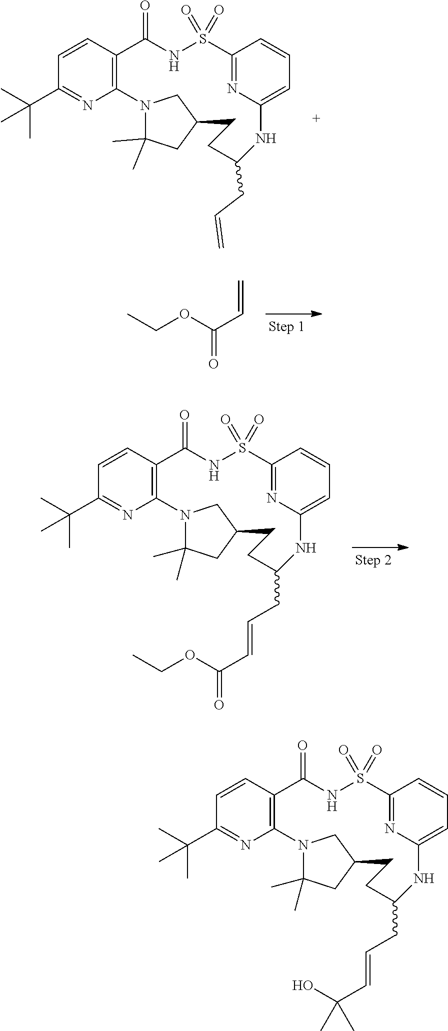

### Step 1: ethyl (2Z)-4-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]but-2-enoate and ethyl (2E)-4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]but-2-enoate

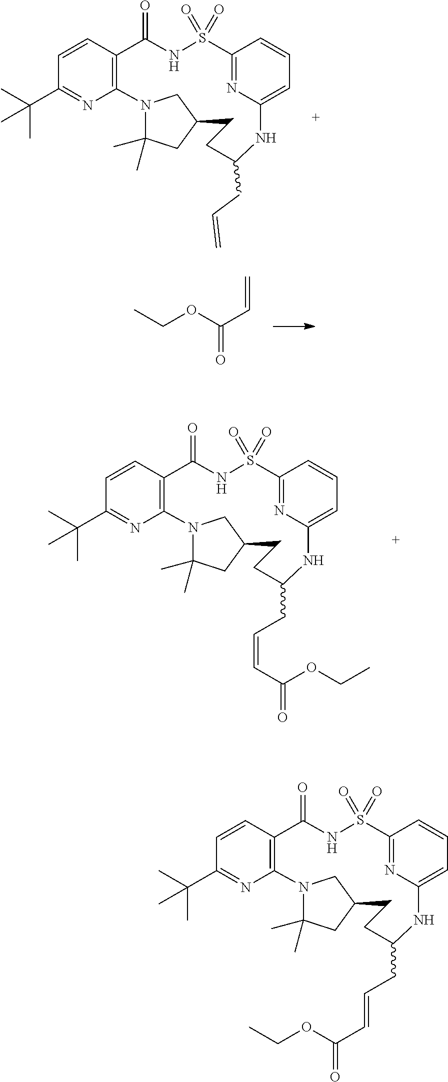

In a 1-mL HPLC vial, (14S)-8-tert-butyl-12,12-dimethyl-17-(prop-2-en-1-yl)-2λ⁶-thia-3,9,11,18,23-pentaazatetracyclo[17.3.1.111,14.05,10]tetracosa-1(23),5(10),6,8,19,21-hexaene-2,2,4-trione (Compound 34 (diastereomer 2), 57.2 mg, 0.1118 mmol) was dissolved in DCE (400 μL), to which ethyl acrylate (50 μL, 0.4615 mmol) and Hoveyda-Grubbs 2nd generation catalyst (8.3 mg, 0.01325 mmol) were added. The vial was capped with a screw-cap septum, and the reaction mixture was stirred at 60° C. for 72 h. It was then cooled to room temperature, and directly purified by silica gel chromatography (4 g of silica) using a gradient eluent of 0 to 50% EtOAc in hexanes to give two products:

Ethyl (2Z)-4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]but-2-enoate (5.6 mg, 9%). ESI-MS m/z calc. 583.28284, found 584.3 (M+1)⁺; Retention time: 2.19 minutes (LC method A).

Ethyl (2E)-4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]but-2-enoate (eluted last, 40.9 mg, 63%). ¹H NMR (400 MHz, DMSO-d₆) δ 12.50 (s, 1H), 7.65-7.54 (m, 2H), 7.15-7.03 (m, 2H), 6.89 (dt, J=15.1, 7.3 Hz, 1H), 6.71 (d, J=8.5 Hz, 1H), 6.63 (d, J=8.0 Hz, 1H), 5.87 (d, J=15.6 Hz, 1H), 4.34-4.23 (m, 1H), 4.06 (q, J=6.8 Hz, 2H), 3.32-3.27 (m, 1H), 3.04 (t, J=8.6 Hz, 1H), 2.69-2.59 (m, 1H), 2.38-2.25 (m, 2H), 2.15-2.01 (m, 1H), 1.79 (dd, J=12.0, 5.3 Hz, 1H), 1.65-1.62 (m, 1H), 1.61 (s, 3H), 1.55-1.49 (m, 2H), 1.48 (s, 3H), 1.34-1.28 (m, 1H), 1.27 (s, 9H), 1.16 (t, J=7.1 Hz, 3H) [Note: A coupling constant of >15 Hz for the 6.89 and 5.87 ppm peaks suggests a trans olefin.]. ESI-MS m/z calc. 583.28284, found 584.3 (M+1)⁺; Retention time: 2.15 minutes (LC method A).

### Step 2: (14S)-8-tert-Butyl-17-[(2E)-4-hydroxy-4-methylpent-2-en-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 23 (diastereomer 2)

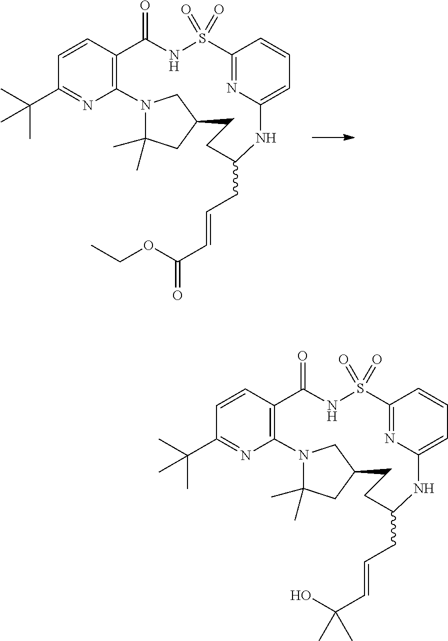 [SMILES: CCOC(=O)/C=C/CC1CC[C@@H]2CN(c3nc(C(C)(C)C)ccc3C(=O)NS(=O)(=O)c3cccc(n3)N1)C(C)(C)C2 ; CC(C)(O)/C=C/CC1CC[C@@H]2CN(c3nc(C(C)(C)C)ccc3C(=O)NS(=O)(=O)c3cccc(n3)N1)C(C)(C)C2]

Stage 1: In a 3-mL vial, ethyl (2E)-4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]but-2-enoate (38.4 mg, 0.06578 mmol) was dissolved in anhydrous THF (300 μL), flushed with nitrogen gas, and sealed with a screw-top septum. Then, a diethyl ether solution of MeMgBr (200 μL of 3.0 M, 0.6000 mmol) was added dropwise. The resulting orange solution was kept at room temperature for 16 h. It was then quenched dropwise with 1 N HCl solution (1 mL), and extracted with EtOAc (2×1 mL). The combined organic extracts was washed with H₂O (2 mL) and saturated aqueous NaCl solution (2 mL), then dried over Na₂SO₄, filtered, and evaporated in vacuo.

Stage 2: The crude product from Step 1 was dissolved in THF (400 μL) and treated with aqueous NaOH (400 μL of 1.0 M, 0.4000 mmol). The resulting mixture was vigorously stirred at 50° C. for 3 h. It was cooled to room temperature, then 1 N HCl solution (3.0 mL) was added, followed by EtOAc (3.0 mL). The phases were vigorously mixed and then allowed to settle into two layers. The organic layer was filtered and purified by reverse-phase preparative chromatography using a C₁₈ column and a gradient eluent of 1 to 99% acetonitrile in water containing 5 mM HCl solution to give (14S)-8-tert-butyl-17-[(2E)-4-hydroxy-4-methylpent-2-en-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (2.2 mg, 6%). ESI-MS m/z calc. 569.3036, found 570.3 (M+1)⁺; Retention time: 2.13 minutes (LC method A).

The compound in the following table was prepared in a manner analogous to that described above, using (14S)-8-tert-butyl-12,12-dimethyl-17-(prop-2-en-1-yl)-2λ⁶-thia-3,9,11,18,23-pentaazatetracyclo[17.3.1.111,14.05,10]tetracosa-1(23),5(10),6,8,19,21-hexaene-2,2,4-trione diastereomer 1, Compound 35, as a starting material:

| | | LCMS | | | |
|---|---|---|---|---|---|
| | | Retention | | | |
| Compound | | Time | Exact | | LCMS |
| Number | Structure | (min) | Mass | M + 1 | Method |
| Compound 24 | 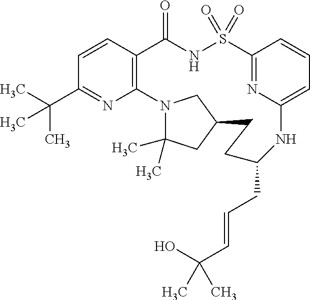 [SMILES: CC(C)(O)/C=C/C[C@@H]1CC[C@@H]2CN(c3nc(C(C)(C)C)ccc3C(=O)NS(=O)(=O)c3cccc(n3)N1)C(C)(C)C2] | 2.11 | 569.304 | 570.3 | LC method A |

### Example 87: Preparation of (14S)-8-tert-Butyl-17-(2-hydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 22 (enantiomer 1), Compound 20 (enantiomer 2), Compound 28 (enantiomer 3), and Compound 27 (enantiomer 4)

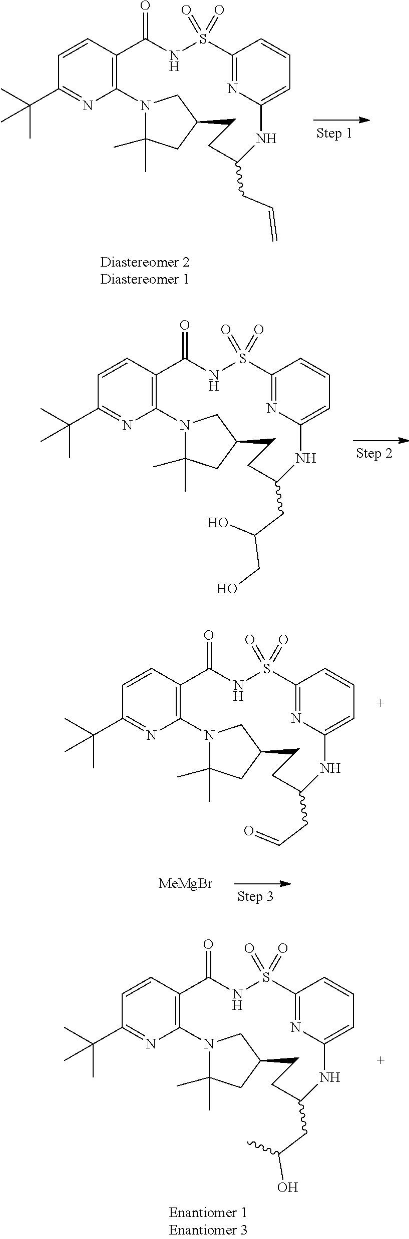 [SMILES: C=CCC1CC[C@@H]2CN(c3nc(C(C)(C)C)ccc3C(=O)NS(=O)(=O)c3cccc(n3)N1)C(C)(C)C2 ; "MeMgBr" ; CC(C)(C)c1ccc2c(n1)N1C[C@@H](CCC(CC(O)CO)Nc3cccc(n3)S(=O)(=O)NC2=O)CC1(C)C ; CC(C)(C)c1ccc2c(n1)N1C[C@@H](CCC(CC=O)Nc3cccc(n3)S(=O)(=O)NC2=O)CC1(C)C ; CC(O)CC1CC[C@@H]2CN(c3nc(C(C)(C)C)ccc3C(=O)NS(=O)(=O)c3cccc(n3)N1)C(C)(C)C2]

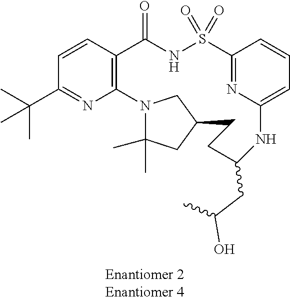 [SMILES: CC(O)CC1CC[C@@H]2CN(c3nc(C(C)(C)C)ccc3C(=O)NS(=O)(=O)c3cccc(n3)N1)C(C)(C)C2]

### Step 1: (14S)-8-tert-Butyl-17-(2,3-dihydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione

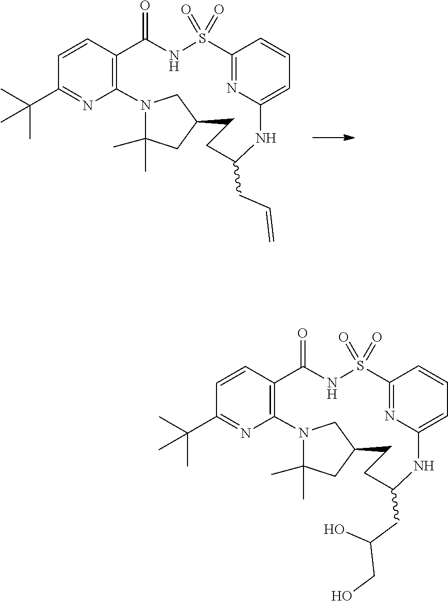 [SMILES: C=CCC1CC[C@@H]2CN(c3nc(C(C)(C)C)ccc3C(=O)NS(=O)(=O)c3cccc(n3)N1)C(C)(C)C2 ; CC(C)(C)c1ccc2c(n1)N1C[C@@H](CCC(CC(O)CO)Nc3cccc(n3)S(=O)(=O)NC2=O)CC1(C)C]

Added 4-methyl-4-oxido-morpholin-4-ium (185 mg, 1.579 mmol) and potassium osmate(VI) dihydrate (32 mg, 0.0868 mmol) to a 1 dram vial under nitrogen. Added (14S)-8-tert-butyl-12,12-dimethyl-17-(prop-2-en-1-yl)-2λ⁶-thia-3,9,11,18,23-pentaazatetracyclo[17.3.1.111,14.05,10]tetracosa-1(23),5(10),6,8,19,21-hexaene-2,2,4-trione (Compound 34 (diastereomer 2), 400 mg, 0.7817 mmol) in acetone (8 mL) followed by water (800 μL). Stirred overnight (resulted in dark black solution). Quenched with sodium thiosulfate and water. Poured into DCM and separated using PTFE frit. Back extracted once. Evaporated off the volatiles. Took up crude in DMSO and purified via preparative HPLC (C₁₈ ACN/Water HCl modifier 15 minutes gradient 0-99% ACN). Concentrated fractions under vacuum to yield (14S)-8-tert-butyl-17-(2,3-dihydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (241.3 mg, 57%). ESI-MS m/z calc. 545.2672, found 546.2 (M+1)⁺; Retention time: 1.65 minutes (LC method A).

### Step 2: 2-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]acetaldehyde

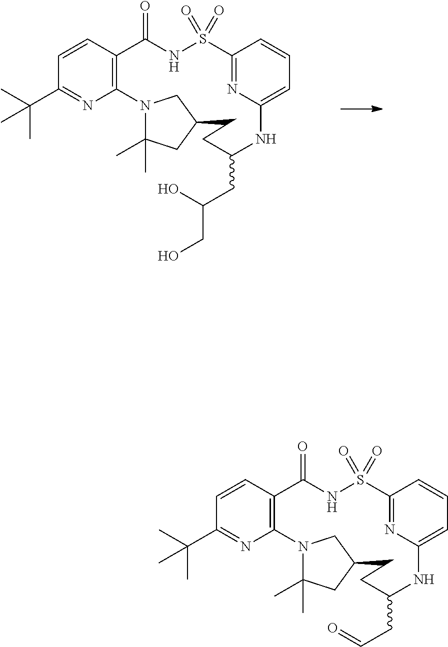 [SMILES: CC(C)(C)c1ccc2c(n1)N1C[C@@H](CCC(CC(O)CO)Nc3cccc(n3)S(=O)(=O)NC2=O)CC1(C)C ; CC(C)(C)c1ccc2c(n1)N1C[C@@H](CCC(CC=O)Nc3cccc(n3)S(=O)(=O)NC2=O)CC1(C)C]

Dissolved (14S)-8-tert-butyl-17-(2,3-dihydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (250 mg, 0.4581 mmol) in dioxane (10 mL):water (2 mL) and 2,6-lutidine (140 μL, 1.209 mmol) under nitrogen. Added periodate (Sodium salt) (210 mg, 0.9818 mmol) and stirred for 1 h at room temperature. Diluted with DCM/1N HCl and separated the layers. Evaporated the solvent before placing under nitrogen and dissolving the crude in THF (2 mL) at 0° C. Quenched with water followed by the addition of DCM/1 N HCl. Separated the layers over a PTFE frit before evaporating the solvent under reduced pressure. Yielded 2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]acetaldehyde (198.5 mg, 84%). ¹H NMR (400 MHz, DMSO-d₆) δ 12.49 (s, 1H), 9.68 (t, J=1.9 Hz, 1H), 7.60 (t, J=7.9 Hz, 1H), 7.32 (dd, J=59.6, 7.5 Hz, 3H), 6.66 (dd, J=48.0, 8.2 Hz, 2H), 3.90 (s, 1H), 3.20 (s, 1H), 2.99-2.61 (m, 2H), 2.36-2.20 (m, 2H), 1.90-1.41 (m, 10H), 1.26 (m, 11H).

### Step 3: (14S)-8-tert-Butyl-17-(2-hydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 22 (enantiomer 1) and (14S)-8-tert-butyl-17-(2-hydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, Compound 20 (enantiomer 2)

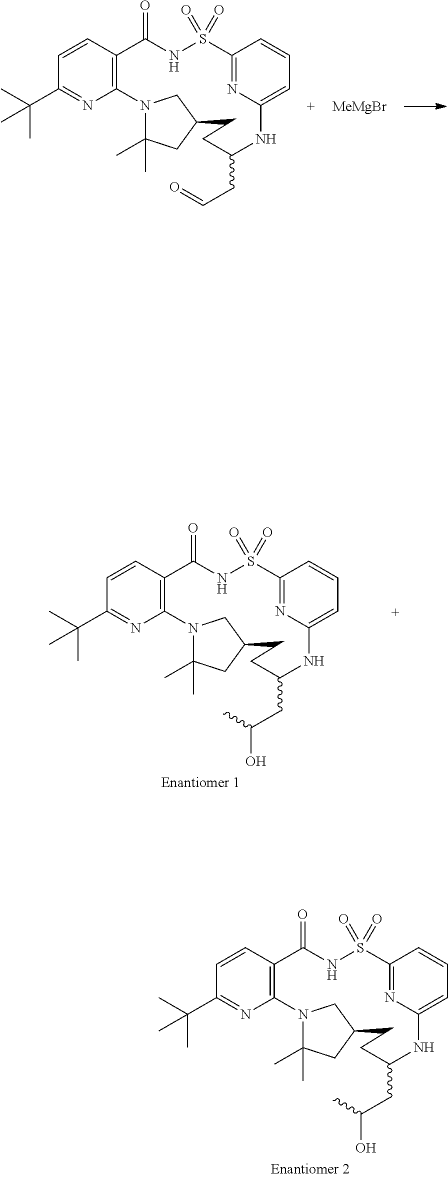

Dissolved 2-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]acetaldehyde (25 mg, 0.04867 mmol) in THF (1 mL) under nitrogen. Cooled to 0° C. Added MeMgBr (25 μL, 0.2159 mmol) and stirred for 2 h. Quenched with saturated ammonium chloride solution and extracted with DCM over a PTFE frit. Evaporated off volatiles before purifying via prep LCMS (1-99% ACN/Water HCl modifier). Two isomers were isolated:

Enantiomer 1, first to elute, peak 1: (14S)-8-tert-Butyl-17-(2-hydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (2.5 mg, 10%). ¹H NMR (400 MHz, Methanol-d₄) δ 7.65 (s, 1H), 7.56 (t, J=7.9 Hz, 1H), 7.31 (d, J=7.2 Hz, 1H), 6.80-6.67 (m, 2H), 3.85 (q, J=6.3 Hz, 1H), 3.69 (s, 1H), 3.29-3.04 (m, 2H), 2.34 (s, 1H), 2.12-1.67 (m, 4H), 1.61 (d, J=17.9 Hz, 9H), 1.39-1.18 (m, 14H). ESI-MS m/z calc. 529.2723, found 530.2 (M+1)⁺; Retention time: 1.86 minutes (LC method A).

Enantomer 2, second to elute, peak 1: (14S)-8-tert-butyl-17-(2-hydroxypropyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (4.2 mg, 16%). ¹H NMR (400 MHz, Methanol-d₄) δ 7.65 (s, 1H), 7.56 (t, J=7.9 Hz, 1H), 7.31 (d, J=7.2 Hz, 1H), 6.80-6.67 (m, 2H), 3.85 (q, J=6.3 Hz, 1H), 3.69 (s, 1H), 3.29-3.04 (m, 2H), 2.34 (s, 1H), 2.12-1.67 (m, 4H), 1.61 (d, J=17.9 Hz, 9H), 1.39-1.18 (m, 14H). ESI-MS m/z calc. 529.2723, found 530.3 (M+1)⁺; Retention time: 1.86 minutes (LC method A).

The compounds in the following table were prepared in a manner analogous to that described above, using (14S)-8-tert-butyl-12,12-dimethyl-17-(prop-2-en-1-yl)-2λ⁶-thia-3,9,11,18,23-pentaazatetracyclo[17.3.1.111,14.05,10]tetracosa-1(23),5(10),6,8,19,21-hexaene-2,2,4-trione (Compound 35 (diastereomer 1)) as a starting material in step 1:

| | | LCMS | | | |
|---|---|---|---|---|---|
| | | Retention | | | |
| Compound | | Time | Exact | | LCMS |
| Number | Structure | (min) | Mass | M + 1 | Method |
| Compound 28 (enantiomer 3, peak 1) | 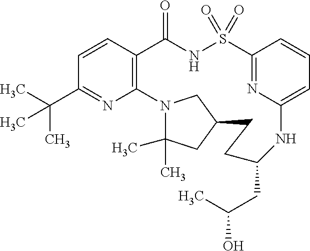 [SMILES: C[C@@H](O)C[C@@H]1CC[C@@H]2CN(c3nc(C(C)(C)C)ccc3C(=O)NS(=O)(=O)c3cccc(n3)N1)C(C)(C)C2] | 1.92 | 529.272 | 530.2 | LC method A |
| Compound 27 (enantiomer 4, peak 2) | 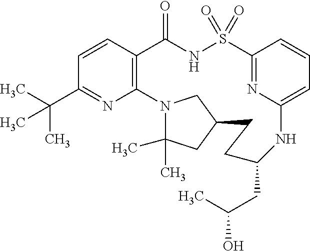 [SMILES: C[C@@H](O)C[C@@H]1CC[C@@H]2CN(c3nc(C(C)(C)C)ccc3C(=O)NS(=O)(=O)c3cccc(n3)N1)C(C)(C)C2] | 1.99 | 529.272 | 530.2 | LC method A |

### Example 88: Preparation of 4-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]butanoic acid, Compound 19

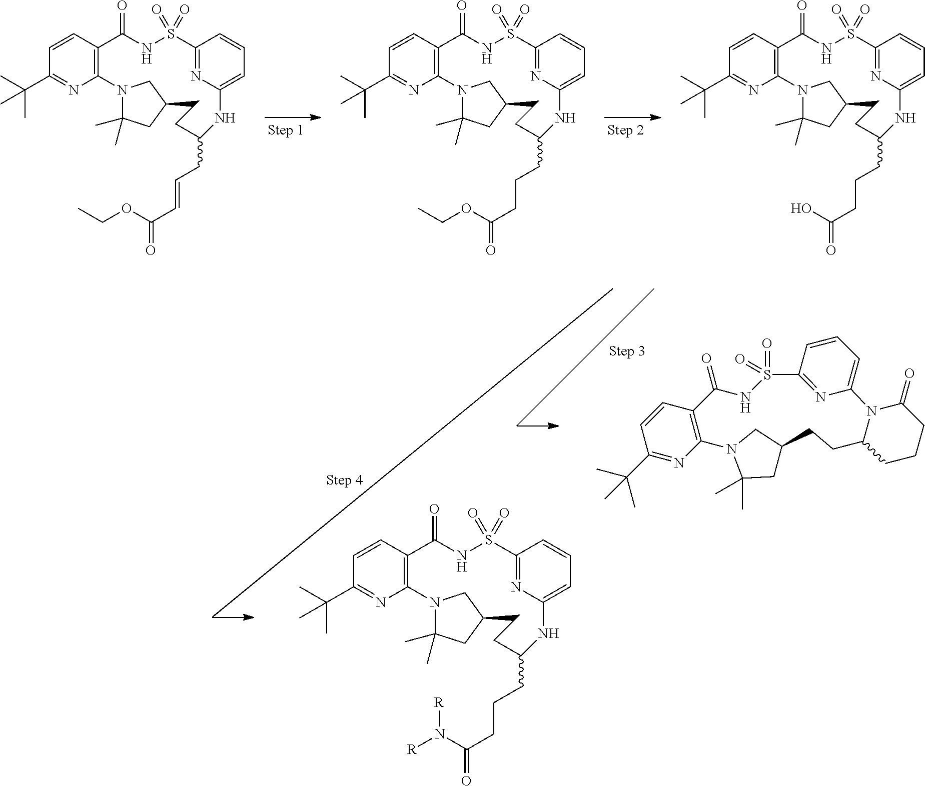

### Step 1: Ethyl 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]butanoate

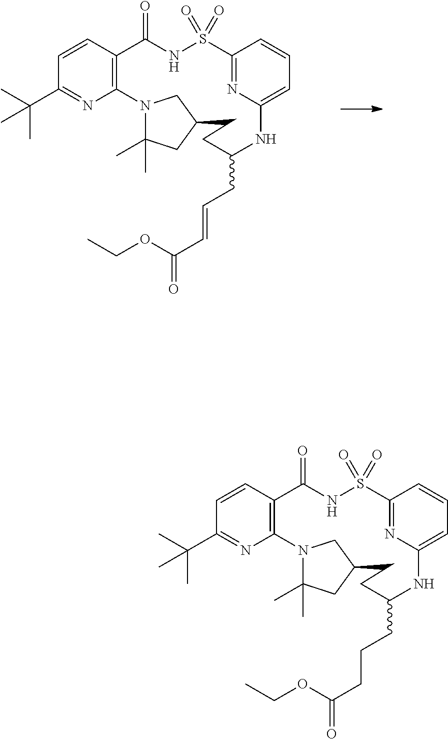 [SMILES: CCOC(=O)/C=C/CC1CC[C@@H]2CN(c3nc(C(C)(C)C)ccc3C(=O)NS(=O)(=O)c3cccc(n3)N1)C(C)(C)C2 ; CCOC(=O)CCCC1CC[C@@H]2CN(c3nc(C(C)(C)C)ccc3C(=O)NS(=O)(=O)c3cccc(n3)N1)C(C)(C)C2]

In a 5-mL microwave vial, ethyl (2E)-4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]but-2-enoate (originating from Compound 34 (diastereomer 2), 293.3 mg, 0.5024 mmol) was dissolved in EtOH (2.0 mL), and sparged with a balloon of nitrogen gas for 5 min. Pd/C (23.5 mg of 10% w/w, 0.02208 mmol) was added, and the resulting mixture was stirred under a balloon of hydrogen (2 L, 79.37 mmol) at room temperature for 1 h then at 50° C. for 18 h. The reaction mixture was then cooled to room temperature, filtered through Celite (rinsed with MeOH), and evaporated to dryness to give an off-white foam, ethyl 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]butanoate (278.4 mg, 95%). ESI-MS m/z calc. 585.29846, found 586.3 (M+1)⁺; Retention time: 2.18 minutes (LC method A).

### Step 2: 4-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]butanoic acid, Compound 19

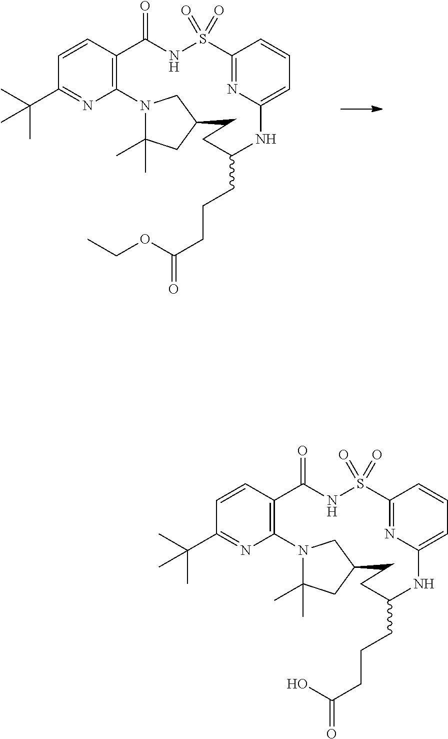 [SMILES: CCOC(=O)CCCC1CC[C@@H]2CN(c3nc(C(C)(C)C)ccc3C(=O)NS(=O)(=O)c3cccc(n3)N1)C(C)(C)C2 ; CC(C)(C)c1ccc2c(n1)N1C[C@@H](CCC(CCCC(=O)O)Nc3cccc(n3)S(=O)(=O)NC2=O)CC1(C)C]

In a 20-mL vial, ethyl 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]butanoate (226.6 mg, 0.3868 mmol) was dissolved in THF (2.0 mL), to which aqueous NaOH (2.0 mL of 1.0 M, 2.000 mmol) was added. The resulting mixture was stirred vigorously at 50° C. for 17 h. The reaction mixture was then cooled to room temperature, quenched with 1 N HCl (5 mL), and extracted with EtOAc (3×3 mL). The combined organic extracts was washed with H₂O (5 mL) and saturated aqueous NaCl solution (5 mL), then dried over Na₂SO₄, filtered, and evaporated in vacuo. This gave an off-white solid: 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]butanoic acid (213.2 mg, 99%) ESI-MS m/z calc. 557.2672, found 558.3 (M+1)⁺; Retention time: 1.94 minutes (LC method A). A small amount (10.5 mg) of material was dissolved in MeOH (500 μL), filtered, and purified by reverse-phase preparative chromatography using a C₁₈ column and a gradient eluent of 1 to 99% MeCN in H₂O containing 5 mM HCl solution to give 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]butanoic acid (7.8 mg). ESI-MS m/z calc. 557.2672, found 558.3 (M+1)⁺; Retention time: 1.94 minutes (LC method A).

### Step 3: (10S)-16-tert-Butyl-12,12-dimethyl-22λ⁶-thia-2,13,15,21,27-pentaazapentacyclo[21.3.1.110,13.02,7.014,19]octacosa-1(26),14(19),15,17,23(27),24-hexaene-3,20,22,22-tetrone, Compound 14

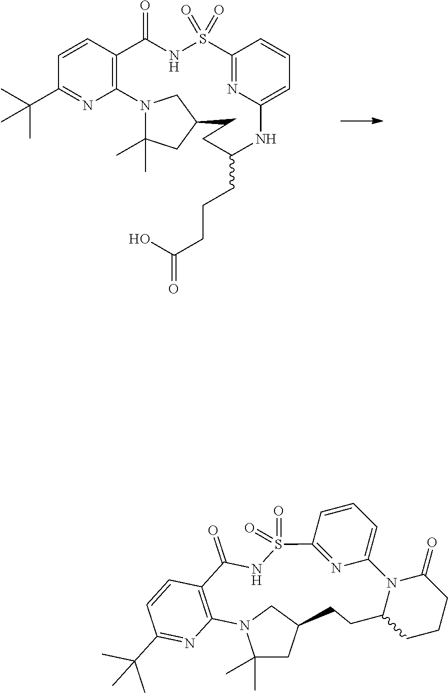 [SMILES: CC(C)(C)c1ccc2c(n1)N1C[C@@H](CCC(CCCC(=O)O)Nc3cccc(n3)S(=O)(=O)NC2=O)CC1(C)C ; CC(C)(C)c1ccc2c(n1)N1C[C@@H](CCC3CCCC(=O)N3c3cccc(n3)S(=O)(=O)NC2=O)CC1(C)C]

In a 1-mL HPLC vial, 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]butanoic acid (6.7 mg, 0.01201 mmol) was dissolved in DMF (200 μL), to which DIPEA (20 μL, 0.1148 mmol) and HATU (7.5 mg, 0.01972 mmol) were added. The resulting solution was stirred at room temperature for 2 h, after which a second portion of HATU (7.5 mg, 0.01972 mmol) was added. The resulting solution was stirred at room temperature for 24 h, after which a third portion of HATU (7.5 mg, 0.01972 mmol) was added. The resulting solution was stirred at room temperature for 24 h, after which it was diluted with MeOH (600 μL), filtered, and purified by reverse-phase preparative chromatography using a C₁₈ column and a gradient eluent of 1 to 99% MeCN in H₂O containing 5 mM HCl solution to give (10S)-16-tert-butyl-12,12-dimethyl-22λ⁶-thia-2,13,15,21,27-pentaazapentacyclo[21.3.1.110,13.02,7.014,19]octacosa-1(26),14(19),15,17,23(27),24-hexaene-3,20,22,22-tetrone (3.6 mg, 56%) ESI-MS m/z calc. 539.25665, found 540.2 (M+1)⁺; Retention time: 1.97 minutes (LC method A).

### Step 4: 4-[(14S)-8-tert-Butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-N,N-dimethylbutanamide, Compound 18

### 4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]-N,N-bis(propan-2-yl)butanamide, Compound 17

### N-tert-butyl-4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]butanamide, Compound 16

### N-benzyl-4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]butanamide, Compound 15

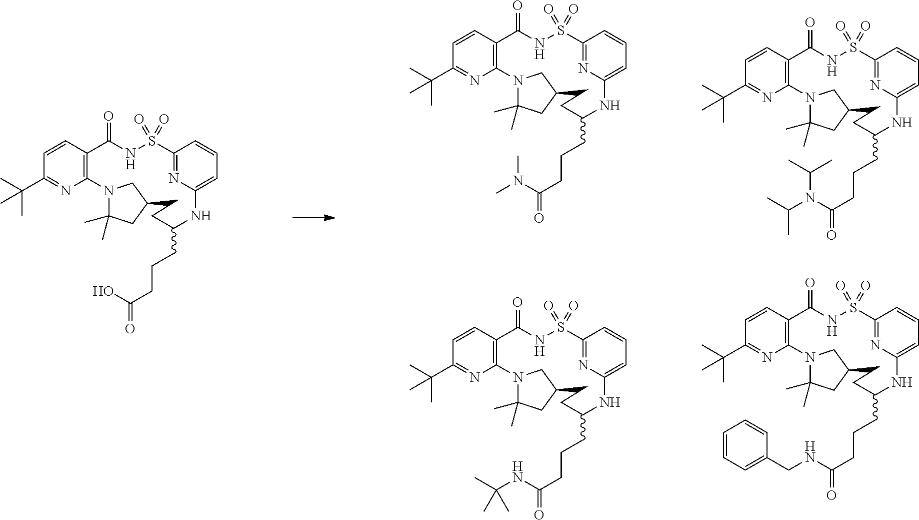 [SMILES: CC(C)(C)c1ccc2c(n1)N1C[C@@H](CCC(CCCC(=O)O)Nc3cccc(n3)S(=O)(=O)NC2=O)CC1(C)C ; CC(C)(C)NC(=O)CCCC1CC[C@@H]2CN(c3nc(C(C)(C)C)ccc3C(=O)NS(=O)(=O)c3cccc(n3)N1)C(C)(C)C2 ; CN(C)C(=O)CCCC1CC[C@@H]2CN(c3nc(C(C)(C)C)ccc3C(=O)NS(=O)(=O)c3cccc(n3)N1)C(C)(C)C2 ; CC(C)(C)c1ccc2c(n1)N1C[C@@H](CCC(CCCC(=O)NCc3ccccc3)Nc3cccc(n3)S(=O)(=O)NC2=O)CC1(C)C ; CC(C)N(C(=O)CCCC1CC[C@@H]2CN(c3nc(C(C)(C)C)ccc3C(=O)NS(=O)(=O)c3cccc(n3)N1)C(C)(C)C2)C(C)C]

In this experiment, 4 reactions were conducted, all in the same manner.

General procedure: In a 1-mL HPLC vial, 4-[(14S)-8-tert-butyl-12,12-dimethyl-2,2,4-tri oxo-2λ⁶-thia-3,9,11,18,23-pentaazatetracyclo[17.3.1.111,14.05,10]tetracosa-1(23),5(10),6,8,19,21-hexaen-17-yl]butanoic acid (9.6 mg, 0.0172 mmol) was dissolved in DMF (100 μL), to which an amine substrate was added, followed by DIPEA (10 μL, 0.0574 mmol) and HATU (12.5 mg, 0.03287 mmol). The resulting mixture was stirred for 5 min or for 90 min, depending on the amine substrate. It was then diluted with MeOH (500 μL), filtered, and purified by reverse-phase preparative chromatography using a C₁₈ column and a gradient eluent of 1 to 99% MeCN in H₂O containing 5 mM HCl solution to give the products:

Amine Substrate:
Me₂NH (hydrochloride salt) (7.0 mg, 0.0858 mmol), reaction time of 5 min;
iPr₂NH (10 μL, 0.0713 mmol), reaction time of 5 min;
t-BuNH₂ (10 μL, 0.0956 mmol), reaction time of 5 min;
BnNH₂ (10 μL, 0.0915 mmol), reaction time of 90 min.

Product List:
4-[(14S)-8-tert-butyl-12,12-dimethyl-2,2,4-tri oxo-2λ⁶-thia-3,9,11,18,23-pentaazatetracyclo[17.3.1.111,14.05,10]tetracosa-1(23),5(10),6,8,19,21-hexaen-17-yl]-N,N-dimethylbutanamide (hydrochloride salt) (5.9 mg, 55%) ESI-MS m/z calc. 584.31445, found 585.3 (M+1)⁺; Retention time: 1.99 minutes (LC method A).
4-[(14S)-8-tert-butyl-12,12-dimethyl-2,2,4-tri oxo-2λ⁶-thia-3,9,11,18,23-pentaazatetracyclo[17.3.1.111,14.05,10]tetracosa-1(23),5(10),6,8,19,21-hexaen-17-yl]-N,N-bis(propan-2-yl)butanamide (hydrochloride salt) (6 mg, 51%) ESI-MS m/z calc. 640.3771, found 641.4 (M+1)⁺; Retention time: 2.3 minutes (LC method A).
N-tert-butyl-4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]butanamide (hydrochloride salt) (7.6 mg, 68%) ESI-MS m/z calc. 612.34576, found 613.4 (M+1)⁺; Retention time: 2.14 minutes (LC method A).
N-benzyl-4-[(14S)-8-tert-butyl-12,12-dimethyl-2,2,4-trioxo-2λ⁶-thia-3,9,11,18,23-pentaazatetracyclo[17.3.1.111,14.05,10]tetracosa-1(23),5(10),6,8,19,21-hexaen-17-yl]butanamide (hydrochloride salt) (3.8 mg, 32%) ESI-MS m/z calc. 646.33014, found 647.3 (M+1)⁺; Retention time: 2.12 minutes (LC method A).

### Example 89: Preparation of 8-tert-Butyl-12,12-dimethyl-17-phenyl-2λ⁶-thia-3,9,11,17-tetraazatetracyclo[17.3.1.111,14.05,10]tetracosa-1(22),5,7,9,19(23),20-hexaene-2,2,4,18-tetrone, Compound 267 (enantiomer 1) and Compound 266 (enantiomer 2)

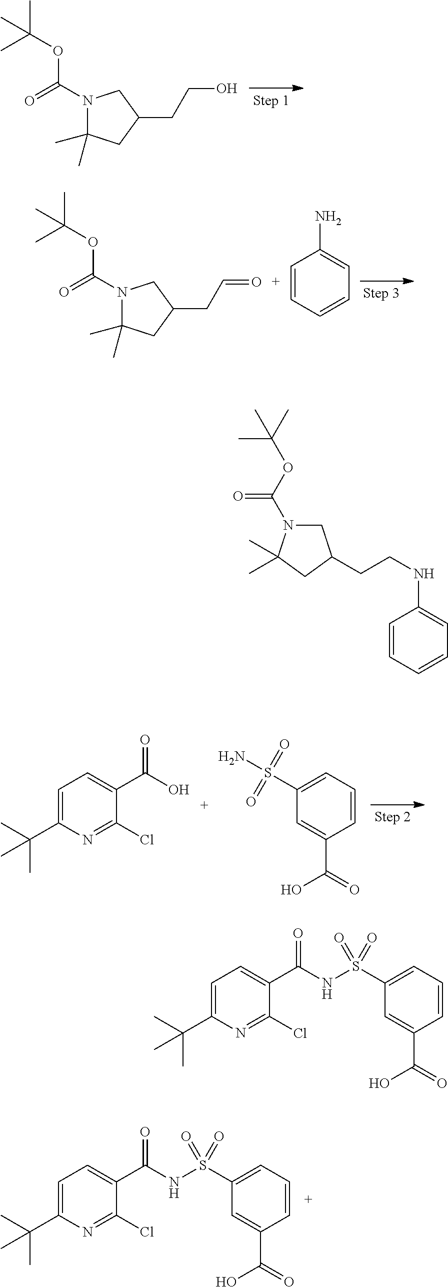

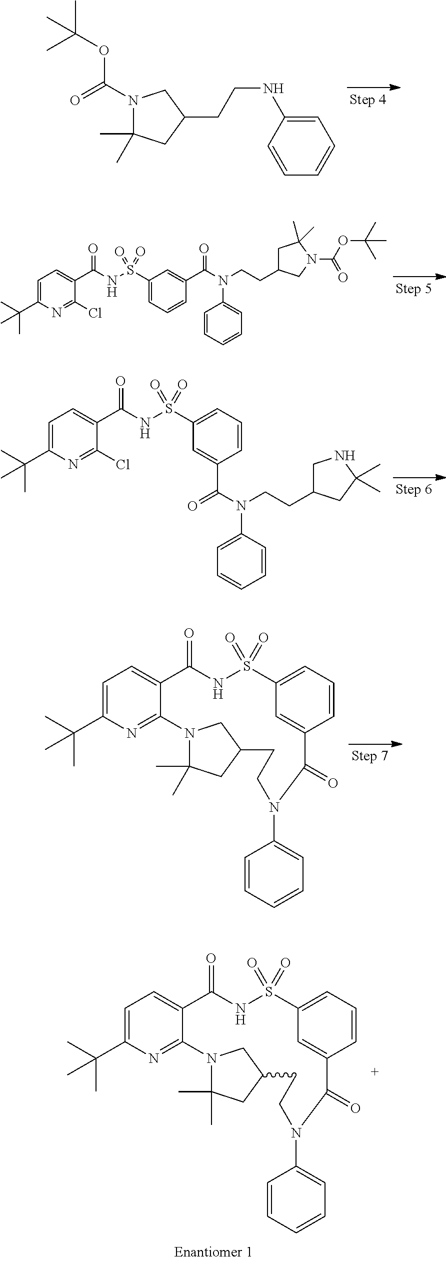

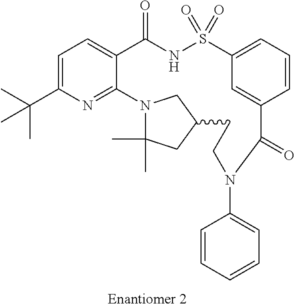 [SMILES: CC(C)(C)c1ccc2c(n1)N1CC(CCN(c3ccccc3)C(=O)c3cccc(c3)S(=O)(=O)NC2=O)CC1(C)C]

### Step 1: tert-Butyl 2,2-dimethyl-4-(2-oxoethyl)pyrrolidine-1-carboxylate

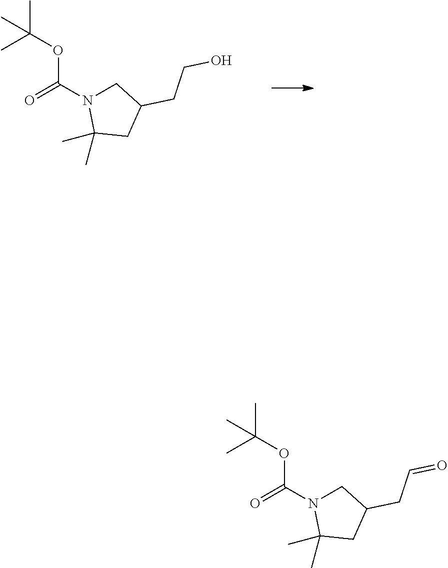 [SMILES: CC(C)(C)OC(=O)N1CC(CCO)CC1(C)C ; CC(C)(C)OC(=O)N1CC(CC=O)CC1(C)C]

To a solution of cold, 0° C., sodium hypochlorite (5.3 mL of 1.2 M, 6.3600 mmol) was added sodium bicarbonate (258 mg, 3.0712 mmol), The resulting solution is slowly added to a suspension of tert-butyl 4-(2-hydroxyethyl)-2,2-dimethyl-pyrrolidine-1-carboxylate (1 g, 4.1094 mmol) with TEMPO (13 mg, 0.0832 mmol) and sodium bromide (42 mg, 0.4082 mmol) in DCM (20 mL) at 0° C., with strong stirring. Once the solution turned brown it was left stirring 10 min and was quenched with ethanol (205.14 mg, 0.26 mL, 4.4529 mmol). The reaction mixture was diluted with DCM (10 mL), the organic phase was separated, dried with sodium sulfate, filtered and concentrated under reduced pressure to provide tert-butyl 2,2-dimethyl-4-(2-oxoethyl)pyrrolidine-1-carboxylate (1.075 g, 99%) as an orange oil. ¹H NMR (400 MHz, CDCl₃) δ 9.78 (s, 1H), 3.88-3.70 (m, 1H), 3.02-2.85 (m, 1H), 2.75-2.39 (m, 3H), 2.01-1.86 (m, 1H), 1.57-1.30 (m, 16H). ESI-MS m/z calc. 241.1678, found 186.4 (M−55)+; Retention time: 1.778 minutes (LC method E).

### Step 2: 3-[(6-tert-Butyl-2-chloro-pyridine-3-carbonyl)sulfamoyl]benzoic acid

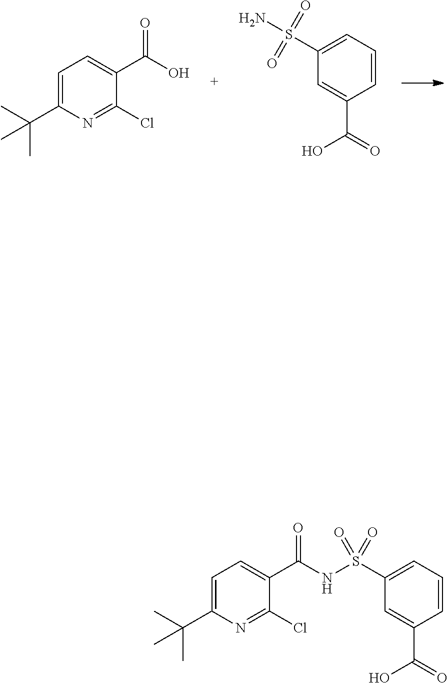 [SMILES: CC(C)(C)c1ccc(C(=O)O)c(Cl)n1 ; NS(=O)(=O)c1cccc(C(=O)O)c1 ; CC(C)(C)c1ccc(C(=O)NS(=O)(=O)c2cccc(C(=O)O)c2)c(Cl)n1]

To a solution of the 6-tert-butyl-2-chloro-pyridine-3-carboxylic acid (500 mg, 2.340 mmol) in THF (4 mL) was added CDI (474 mg, 2.923 mmol) (recrystallized from THF) and the mixture was stirred at rt for 1 h then 3-sulfamoylbenzoic acid (470.8 mg, 2.340 mmol) was added followed by DBU (1.448 mL, 9.683 mmol) and the resulting mixture was stirred for 1 h at room temperature. Diluted the mixture with EtOAc and 1N HCl, separated and washed the aqueous layer once more with EtOAc. Combined the organic phases, dried (sodium sulfate), filtered and concentrated to a yellow oil which slowly crystallized to the crude product, 3-[(6-tert-butyl-2-chloro-pyridine-3-carbonyl)sulfamoyl]benzoic acid (1.2432 g, 86%). ESI-MS m/z calc. 396.05466, found 397.2 (M+1)⁺; Retention time: 0.58 minutes (LC method D).

### Step 3: tert-Butyl 4-(2-anilinoethyl)-2,2-dimethyl-pyrrolidine-1-carboxylate

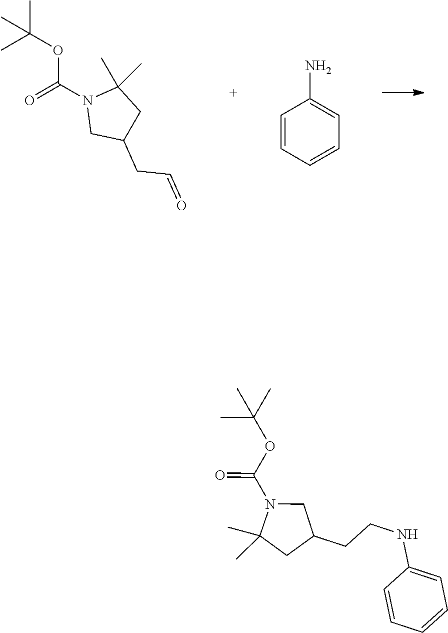 [SMILES: CC(C)(C)OC(=O)N1CC(CC=O)CC1(C)C ; Nc1ccccc1 ; CC(C)(C)OC(=O)N1CC(CCNc2ccccc2)CC1(C)C]

To a solution of tert-butyl 2,2-dimethyl-4-(2-oxoethyl)pyrrolidine-1-carboxylate (680.7 mg, 2.821 mmol) in dichloromethane (10.21 mL) at 0° C. were added aniline (308.4 μL, 3.384 mmol) and Na(OAc)₃BH (1.197 g, 5.648 mmol). Stirred 2 h then diluted with DCM and MeOH and filtered over celite. The filtrate was concentrated by rotary evaporation, dissolved in EtOAc and washed with saturated aqueous sodium bicarbonate (1×), brine (1×), dried over sodium sulfate, filtered and concentrated to a clear oil which was purified by silica gel chromatography using a shallow gradient from 100% hexanes to 100% EtOAc giving tert-butyl 4-(2-anilinoethyl)-2,2-dimethyl-pyrrolidine-1-carboxylate as a clear oil (853.5 mg, 90%). ESI-MS m/z calc. 318.23074, found 319.2 (M+1)⁺; Retention time: 0.54 minutes (LC method D).

### Step 4: tert-Butyl 4-[2-(N-[3-[(6-tert-butyl-2-chloro-pyridine-3-carbonyl)sulfamoyl]benzoyl]anilino)ethyl]-2,2-dimethyl-pyrrolidine-1-carboxylate

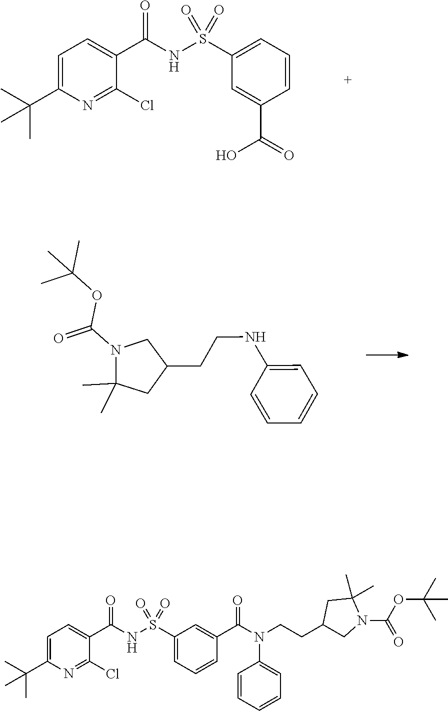 [SMILES: CC(C)(C)c1ccc(C(=O)NS(=O)(=O)c2cccc(C(=O)O)c2)c(Cl)n1 ; CC(C)(C)OC(=O)N1CC(CCNc2ccccc2)CC1(C)C ; CC(C)(C)OC(=O)N1CC(CCN(C(=O)c2cccc(S(=O)(=O)NC(=O)c3ccc(C(C)(C)C)nc3Cl)c2)c2ccccc2)CC1(C)C]

tert-Butyl 4-(2-anilinoethyl)-2,2-dimethyl-pyrrolidine-1-carboxylate (409.8 mg, 1.287 mmol) and 3-[(6-tert-butyl-2-chloro-pyridine-3-carbonyl)sulfamoyl]benzoic acid (260.8 mg, 0.6572 mmol) were dissolved in DCM (18 mL) in a flask and to this mixture was added sequentially EDC (215.5 mg, 1.124 mmol), DMAP (96.34 mg, 0.7886 mmol) and finally DIEA (254.9 mg, 1.972 mmol). The mixture showed some conversion after 30 min and was stirred overnight but did not progress further. Added 3-(ethyliminomethyleneamino)-N,N-dimethyl-propan-1-amine (hydrochloride salt) (215.5 mg, 1.124 mmol) (from a fresh bottle), DMAP (96.34 mg, 0.7886 mmol) and DIEA (254.9 mg, 1.972 mmol), heated to 65° C. and stirred 2 h, 50% conversion. Added DMAP (80.29 mg, 0.6572 mmol) and DIEA (127.4 mg, 0.9857 mmol), heated to 75° C. and stirred for 3.5 h. Cooled to room temperature then diluted with 1N HCl, washed with EtOAc (2 x, added trace MeOH to improve solubility), combined the organic layers, dried (sodium sulfate), filtered and concentrated to a clear oil. Purified by silica gel chromatography using a shallow gradient from 100% hexanes to 100% EtOAc giving the product contaminated with the acid starting material. This material was dissolved in EtOAc, washed with 1N NaOH (2×), dried (sodium sulfate), filtered and concentrated to a clear oil which was taken directly to the next step, tert-butyl 4-[2-(N-[3-[(6-tert-butyl-2-chloro-pyridine-3-carbonyl)sulfamoyl]benzoyl]anilino)ethyl]-2,2-dimethyl-pyrrolidine-1-carboxylate (120.3 mg, 26%). ESI-MS m/z calc. 696.27484, found 697.2 (M+1)⁺; Retention time: 0.83 minutes (LC method D).

### Step 5: 6-tert-Butyl-2-chloro-N-[3-[2-(5,5-dimethylpyrrolidin-3-yl)ethyl-phenyl-carbamoyl]phenyl]sulfonyl-pyridine-3-carboxamide

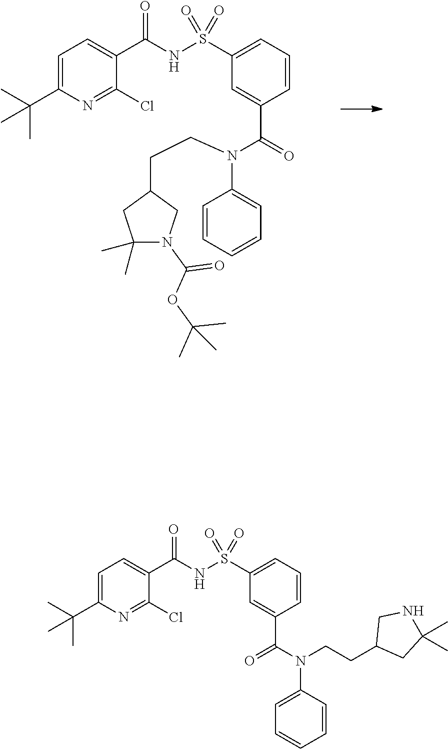 [SMILES: CC(C)(C)OC(=O)N1CC(CCN(C(=O)c2cccc(S(=O)(=O)NC(=O)c3ccc(C(C)(C)C)nc3Cl)c2)c2ccccc2)CC1(C)C ; CC1(C)CC(CCN(C(=O)c2cccc(S(=O)(=O)NC(=O)c3ccc(C(C)(C)C)nc3Cl)c2)c2ccccc2)CN1]

tert-Butyl 4-[2-(N-[3-[(6-tert-butyl-2-chloro-pyridine-3-carbonyl)sulfamoyl]benzoyl]anilino)ethyl]-2,2-dimethyl-pyrrolidine-1-carboxylate (120.3 mg, 0.1725 mmol) was dissolved in DCM (525 μL) and to the mixture was added TFA (532 μL, 6.905 mmol) and the mixture was stirred at room temperature for 1 h. Concentrated mixture to dryness under reduced pressure, added 10 mL of diethyl ether and removed volatiles by rotary evaporation. Repeated this evaporation from 10 mL of diethyl ether twice more then dried under high vacuum for 15 min giving 6-tert-butyl-2-chloro-N-[3-[2-(5,5-dimethylpyrrolidin-3-yl)ethyl-phenyl-carbamoyl]phenyl]sulfonyl-pyridine-3-carboxamide (trifluoroacetate salt) as a white solid (143.5 mg, 100%). ESI-MS m/z calc. 596.2224, found 597.1 (M+1)⁺; Retention time: 0.51 minutes (LC method D).

### Step 6: 8-tert-Butyl-12,12-dimethyl-17-phenyl-2λ⁶-thia-3,9,11,17-tetraazatetracyclo[17.3.1.111,14.05,10]tetracosa-1(22),5,7,9,19(23),20-hexaene-2,2,4,18-tetrone

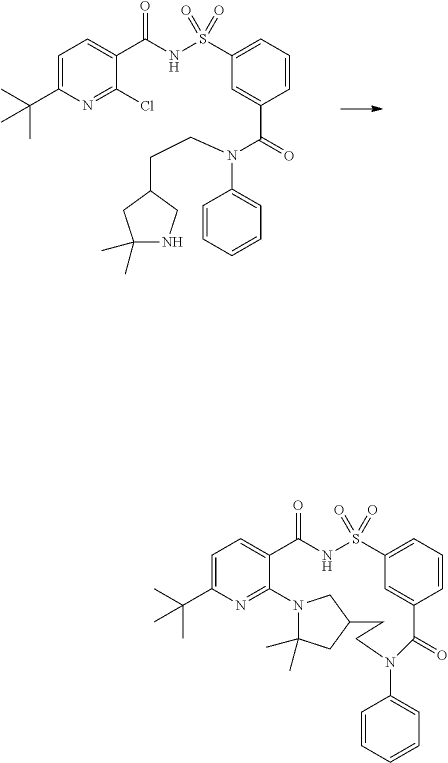 [SMILES: CC1(C)CC(CCN(C(=O)c2cccc(S(=O)(=O)NC(=O)c3ccc(C(C)(C)C)nc3Cl)c2)c2ccccc2)CN1 ; CC(C)(C)c1ccc2c(n1)N1CC(CCN(c3ccccc3)C(=O)c3cccc(c3)S(=O)(=O)NC2=O)CC1(C)C]

To a solution of 6-tert-butyl-2-chloro-N-[3-[2-(5,5-dimethylpyrrolidin-3-yl)ethyl-phenyl-carbamoyl]phenyl]sulfonyl-pyridine-3-carboxamide (Trifluoroacetate salt) (143.5 mg, 0.1721 mmol) in NMP (7 mL) was added potassium carbonate (167 mg, 1.208 mmol). The mixture was purged with nitrogen for 5 min. The mixture was heated at 165° C. and stirred for 3 days, then cesium fluoride (31 mg, 0.2070 mmol) was added followed by 3 Å molecular sieves and the mixture was capped, heated to 180° C. and stirred overnight. Cooled to rt, filtered off the molecular sieves eluting with EtOAc then diluted the filtrate with EtOAc, washed with 1N HCl, dried (sodium sulfate), filtered and concentrated to a brown oil which was filtered and purified using a reverse phase HPLC-MS method using a Luna C₁₈ column (75×30 mm, 5 μm particle size) sold by Phenomenex (pn: 00C-4252-U0-AX), and a dual gradient run from 1-99% mobile phase B over 15.0 minutes (mobile phase A=H₂O (5 mM HCl), mobile phase B=acetonitrile, flow rate=50 mL/min, injection volume=950 μL and column temperature=25° C.) giving as a white solid, 8-tert-butyl-12,12-dimethyl-17-phenyl-2λ⁶-thia-3,9,11,17-tetraazatetracyclo[17.3.1.111,14.05,10]tetracosa-1(22),5,7,9,19(23),20-hexaene-2,2,4,18-tetrone (28 mg, 29%). ESI-MS m/z calc. 560.2457, found 561.2 (M+1)⁺; Retention time: 0.76 minutes (LC method D).

### Step 7: 8-tert-Butyl-12,12-dimethyl-17-phenyl-2λ⁶-thia-3,9,11,17-tetraazatetracyclo[17.3.1.111,14.05,10]tetracosa-1(22),5,7,9,19(23),20-hexaene-2,2,4,18-tetrone, Compound 267 (enantiomer 1), and 8-tert-butyl-12,12-dimethyl-17-phenyl-2λ⁶-thia-3,9,11,17-tetraazatetracyclo[17.3.1.111,14.05,10]tetracosa-1(22),5,7,9,19(23),20-hexaene-2,2,4,18-tetrone, Compound 266 (enantiomer 2)

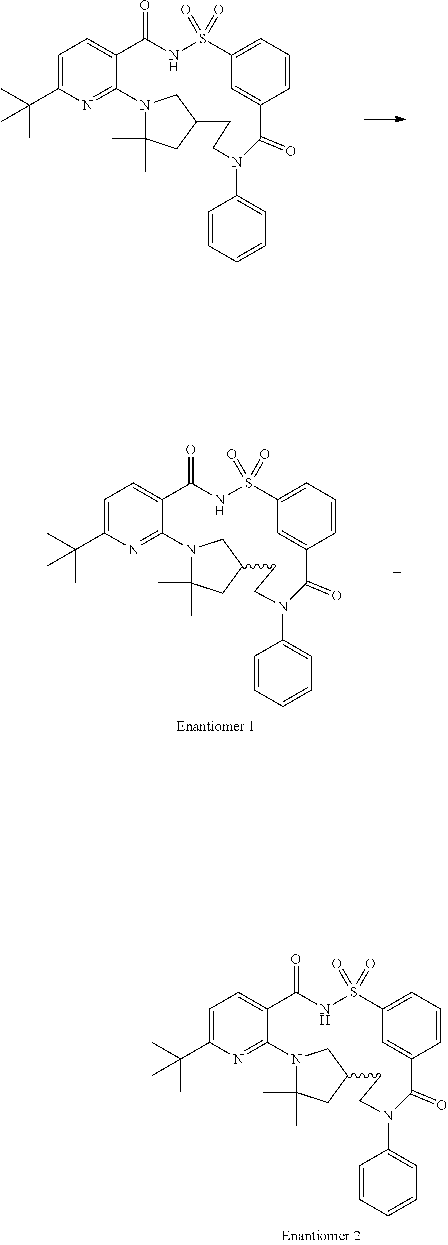 [SMILES: CC(C)(C)c1ccc2c(n1)N1CC(CCN(c3ccccc3)C(=O)c3cccc(c3)S(=O)(=O)NC2=O)CC1(C)C ; CC(C)(C)c1ccc2c(n1)N1CC(CCN(c3ccccc3)C(=O)c3cccc(c3)S(=O)(=O)NC2=O)CC1(C)C ; CC(C)(C)c1ccc2c(n1)N1CC(CCN(c3ccccc3)C(=O)c3cccc(c3)S(=O)(=O)NC2=O)CC1(C)C]

Subjected racemic 8-tert-butyl-12,12-dimethyl-17-phenyl-2λ⁶-thia-3,9,11,17-tetraazatetracyclo[17.3.1.111,14.05,10]tetracosa-1(22),5,7,9,19(23),20-hexaene-2,2,4,18-tetrone (28 mg, 0.04994 mmol) to chiral separation by SFC chromatography using a Regis-[R,R]-Whelk-O (250×10 mm column, 5 μm particle size) with 44% MeOH/56% CO₂ mobile phase at 10 mL/min over 14.0 minutes (injection volume=70 μL of 23 mg/mL solution in 78/22 MeOH/DMSO giving two isomers, both as white solids:

Enantiomer 1, first to elute: 8-tert-Butyl-12,12-dimethyl-17-phenyl-2λ⁶-thia-3,9,11,17-tetraazatetracyclo[17.3.1.111,14.05,10]tetracosa-1(22),5,7,9,19(23),20-hexaene-2,2,4,18-tetrone (6.1 mg, 44%). ¹H NMR (500 MHz, DMSO-d₆) δ 12.60 (s, 1H), 8.21 (s, 2H), 7.83 (s, 2H), 7.52 (s, 1H), 7.46 (s, 4H), 7.34 (s, 1H), 6.65 (d, J=7.9 Hz, 1H), 3.69 (s, 2H), 2.72 (d, J=17.3 Hz, 1H), 1.98 (s, 1H), 1.84 (s, 1H), 1.71 (s, 1H), 1.52 (s, 3H), 1.46 (s, 3H), 1.36 (s, 1H), 1.26 (s, 9H), 1.13 (s, 1H), one H obscured in NMR. ESI-MS m/z calc. 560.2457, found 561.5 (M+1)⁺; Retention time: 2.11 minutes (LC method A).

Enantiomer 2, second to elute: 8-tert-Butyl-12,12-dimethyl-17-phenyl-2λ⁶-thia-3,9,11,17-tetraazatetracyclo[17.3.1.111,14.05,10]tetracosa-1(22),5,7,9,19(23),20-hexaene-2,2,4,18-tetrone (6.7 mg, 48%). ¹H NMR (500 MHz, DMSO-d₆) δ 12.56 (s, 1H), 8.21 (s, 2H), 7.82 (d, J=8.3 Hz, 2H), 7.52 (s, 1H), 7.46 (s, 4H), 7.34 (s, 1H), 6.64 (d, J=7.9 Hz, 1H), 3.69 (s, 2H), 2.72 (d, J=17.1 Hz, 1H), 1.97 (s, 1H), 1.83 (s, 1H), 1.70 (s, 1H), 1.52 (s, 3H), 1.45 (s, 3H), 1.36 (d, J=5.9 Hz, 1H), 1.26 (s, 9H), 1.13 (s, 1H), one H obscured in NMR. ESI-MS m/z calc. 560.2457, found 561.5 (M+1)⁺; Retention time: 2.11 minutes (LC method A).

### Example 90: Preparation of (18S)-21-(4-tert-Butylpyridin-2-yl)-11,16,16-trimethyl-2λ⁶-thia-3,13,15,22,27-pentaazapentacyclo[21.3.1.115,18.05,14.07,12]octacosa-1(26),5(14),6,8,10,12,23(27),24-octaene-2,2,4-trione, Compound 4 (diastereomer 1), Compound 3 (diastereomer 2)

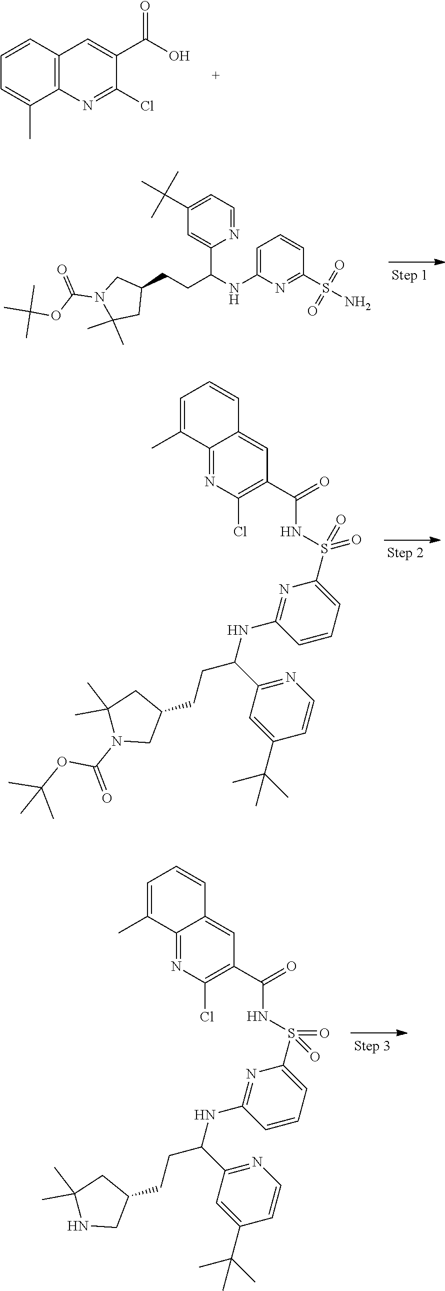 [SMILES: Cc1cccc2cc(C(=O)O)c(Cl)nc12 ; CC(C)(C)OC(=O)N1C[C@@H](CCC(Nc2cccc(S(N)(=O)=O)n2)c2cc(C(C)(C)C)ccn2)CC1(C)C ; Cc1cccc2cc(C(=O)NS(=O)(=O)c3cccc(NC(CC[C@@H]4CN(C(=O)OC(C)(C)C)C(C)(C)C4)c4cc(C(C)(C)C)ccn4)n3)c(Cl)nc12 ; Cc1cccc2cc(C(=O)NS(=O)(=O)c3cccc(NC(CC[C@@H]4CNC(C)(C)C4)c4cc(C(C)(C)C)ccn4)n3)c(Cl)nc12]

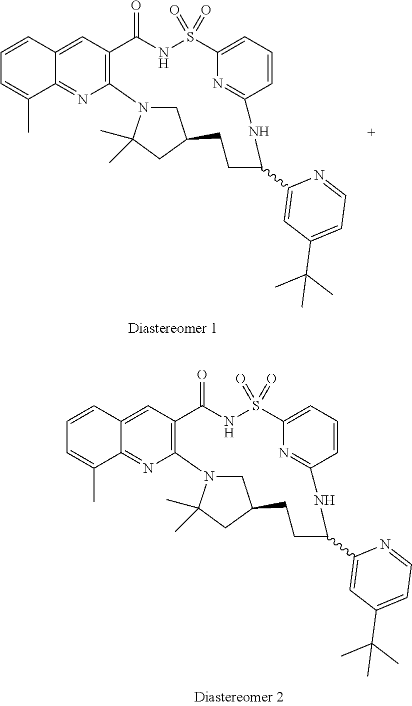 [SMILES: Cc1cccc2cc3c(nc12)N1C[C@@H](CCC(c2cc(C(C)(C)C)ccn2)Nc2cccc(n2)S(=O)(=O)NC3=O)CC1(C)C ; Cc1cccc2cc3c(nc12)N1C[C@@H](CCC(c2cc(C(C)(C)C)ccn2)Nc2cccc(n2)S(=O)(=O)NC3=O)CC1(C)C]

### Step 1: tert-Butyl (4S)-4-[3-(4-tert-butyl-2-pyridyl)-3-[[6-[(2-chloro-8-methyl-quinoline-3-carbonyl)sulfamoyl]-2-pyridyl]amino]propyl]-2,2-dimethyl-pyrrolidine-1-carboxylate

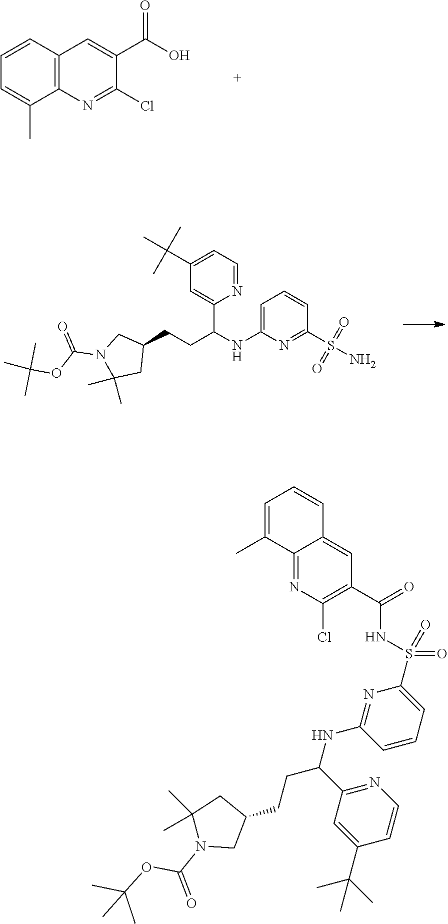 [SMILES: Cc1cccc2cc(C(=O)O)c(Cl)nc12 ; CC(C)(C)OC(=O)N1C[C@@H](CCC(Nc2cccc(S(N)(=O)=O)n2)c2cc(C(C)(C)C)ccn2)CC1(C)C ; Cc1cccc2cc(C(=O)NS(=O)(=O)c3cccc(NC(CC[C@@H]4CN(C(=O)OC(C)(C)C)C(C)(C)C4)c4cc(C(C)(C)C)ccn4)n3)c(Cl)nc12]

2-Chloro-8-methyl-quinoline-3-carboxylic acid (92.5 mg, 0.4173 mmol) was dissolved in THF (2.5 mL) and CDI (130 mg, 0.8017 mmol) was added. The reaction was stirred under nitrogen for 5 h. At this point, tert-butyl (4S)-4-[3-(4-tert-butyl-2-pyridyl)-3-[(6-sulfamoyl-2-pyridyl)amino]propyl]-2,2-dimethyl-pyrrolidine-1-carboxylate (226.8 mg, 0.4156 mmol) and DBU (300 μL, 2.006 mmol) were added and the reaction was stirred an additional 16 h. The reaction was partitioned between ethyl acetate and a 1M HCl solution. The organics were separated, washed with brine, dried over sodium sulfate and evaporated. The crude material was purified by reverse-phase HPLC utilizing a gradient of 10-99% acetonitrile in 5 mM aqueous HCl to yield tert-butyl (4S)-4-[3-(4-tert-butyl-2-pyridyl)-3-[[6-[(2-chloro-8-methyl-quinoline-3-carbonyl)sulfamoyl]-2-pyridyl]amino]propyl]-2,2-dimethyl-pyrrolidine-1-carboxylate (hydrochloride salt) (212.2 mg, 65%) as a yellow solid. ESI-MS m/z calc. 748.3174, found 749.6 (M+1)⁺; Retention time: 0.72 minutes (LC method A).

### Step 2: N-[[6-[[1-(4-tert-Butyl-2-pyridyl)-3-[(3S)-5,5-dimethylpyrrolidin-3-yl]propyl]amino]-2-pyridyl]sulfonyl]-2-chloro-8-methyl-quinoline-3-carboxamide

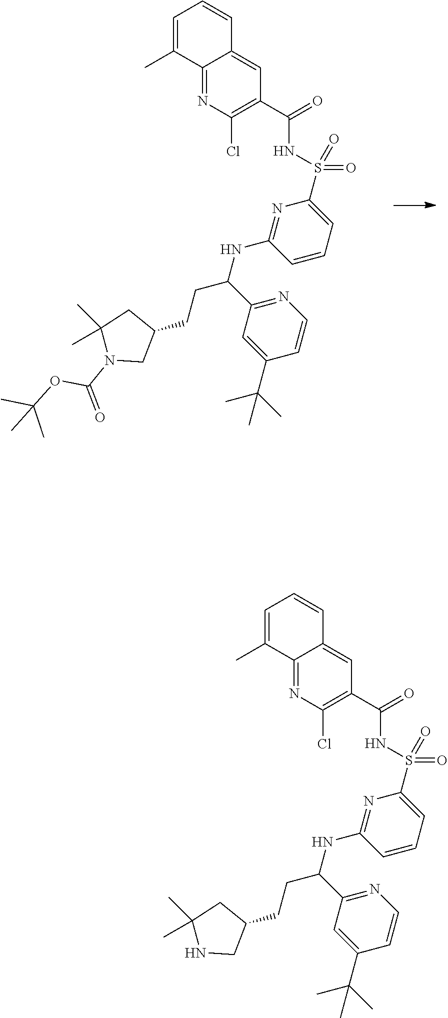 [SMILES: Cc1cccc2cc(C(=O)NS(=O)(=O)c3cccc(NC(CC[C@@H]4CN(C(=O)OC(C)(C)C)C(C)(C)C4)c4cc(C(C)(C)C)ccn4)n3)c(Cl)nc12 ; Cc1cccc2cc(C(=O)NS(=O)(=O)c3cccc(NC(CC[C@@H]4CNC(C)(C)C4)c4cc(C(C)(C)C)ccn4)n3)c(Cl)nc12]

tert-Butyl (4S)-4-[3-(4-tert-butyl-2-pyridyl)-3-[[6-[(2-chloro-8-methyl-quinoline-3-carbonyl)sulfamoyl]-2-pyridyl]amino]propyl]-2,2-dimethyl-pyrrolidine-1-carboxylate (hydrochloride salt) (212 mg, 0.2698 mmol) was dissolved in a mixture of dichloromethane (2 mL): HCl in dioxane (1 mL of 4 M, 4.000 mmol) and stirred at room temperature for 3 h. The reaction was evaporated and further dried to give N-[[6-[[1-(4-tert-butyl-2-pyridyl)-3-[(3S)-5,5-dimethylpyrrolidin-3-yl]propyl]amino]-2-pyridyl]sulfonyl]-2-chloro-8-methyl-quinoline-3-carboxamide (dihydrochloride salt) (194 mg, 100%) as a white solid. Retention time: 0.72 minutes ESI-MS m/z calc. 648.26495, found 649.6 (M+1)⁺; Retention time: 0.5 minutes (LC method D).

### Step 3: (185)-21-(4-tert-Butylpyridin-2-yl)-11,16,16-trimethyl-2λ⁶-thia-3,13,15,22,27-pentaazapentacyclo[21.3.1.115,18.05,14.07,12]octacosa-1(26),5(14),6,8,10,12,23(27),24-octaene-2,2,4-trione, Compound 4 (diastereomer 1) and (18S)-21-(4-tert-Butylpyridin-2-yl)-11,16,16-trimethyl-2λ⁶-thia-3,13,15,22,27-pentaazapentacyclo[21.3.1.115,18.05,14.07,12]octacosa-1(26),5(14),6,8,10,12,23(27),24-octaene-2,2,4-trione, Compound 3 (diastereomer 2)

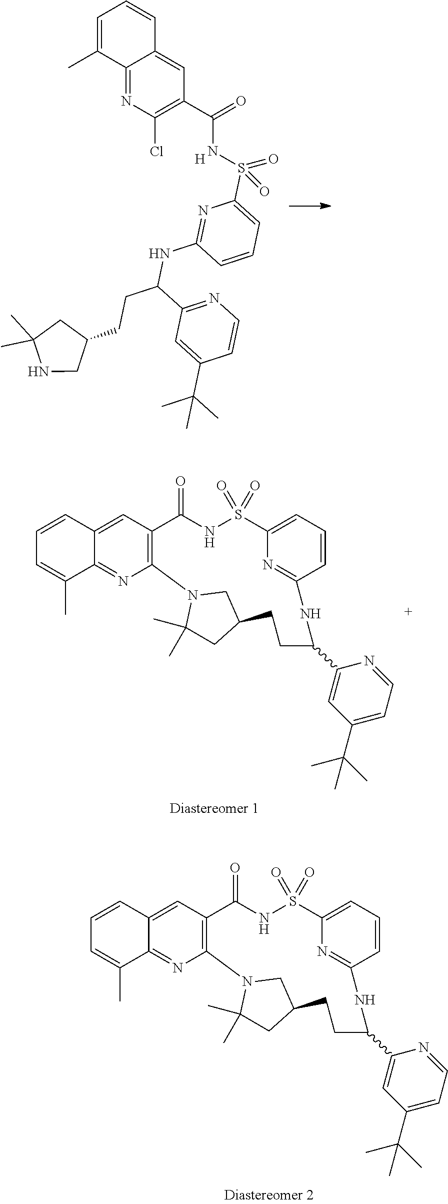

N-[[6-[[1-(4-tert-Butyl-2-pyridyl)-3-[(3S)-5,5-dimethylpyrrolidin-3-yl]propyl]amino]-2-pyridyl]sulfonyl]-2-chloro-8-methyl-quinoline-3-carboxamide (dihydrochloride salt) (194 mg, 0.2686 mmol) and K₂CO₃ (277.5 mg, 2.008 mmol) were combined in NMP (3 mL) and heated at 150° C. for 16 h. The reaction was partitioned between ethyl acetate and a 10% citric acid solution. The organics were separated, washed with brine, dried over sodium sulfate and evaporated. The crude material was purified by reverse-phase HPLC utilizing a gradient of 10-99% acetonitrile in 5 mM aqueous HCl to yield two stereoisomers:

Diastereomer 1, first to elute, peak 1: (18S)-21-(4-tert-Butylpyridin-2-yl)-11,16,16-trimethyl-2λ⁶-thia-3,13,15,22,27-pentaazapentacyclo[21.3.1.115,18.05,14.07,12]octacosa-1(26),5 (14),6,8,10,12,23 (27),24-octaene-2,2,4-trione (dihydrochloride salt) (33.9 mg, 18%). ESI-MS m/z calc. 612.28827, found 613.7 (M+1)⁺; Retention time: 1.45 minutes (LC method A).

Diastereomer 2, second to elute, peak 2: (18S)-21-(4-tert-Butylpyridin-2-yl)-11,16,16-trimethyl-2λ⁶-thia-3,13,15,22,27-pentaazapentacyclo[21.3.1.115,18.05,14.07,12]octacosa-1(26),5 (14),6,8,10,12,23 (27),24-octaene-2,2,4-trione (dihydrochloride salt) (25.8 mg, 13%). ESI-MS m/z calc. 612.28827, found 613.5 (M+1)⁺; Retention time: 1.59 minutes (LC method A).

The following is a list of chloroquinoline reagents that are commercially available:
2-Chloroquinoline-3-carboxylic acid
2-Chloro-7-methyl-quinoline-3-carboxylic acid
2-Chloro-6-methyl-quinoline-3-carboxylic acid.

The compounds in the following table were prepared in a manner analogous to that described above, using commercially available chloroquinoline reagents given in the table above. For each prepared diastereomeric pair, separation of both components was achieved by reverse-phase HPLC utilizing a gradient of 10-99% acetonitrile in 5 mM aqueous HCl. The diastereomer 1 was the first isomer to elute during the separation procedure. The diastereomer 2 was the second isomer to elute.

| | | LCMS | | | |
|---|---|---|---|---|---|
| | | Retention | | | |
| Compound | | Time | Exact | | LCMS |
| Number | Structure | (min) | Mass | M + 1 | Method |
| Compound 6 (Diastereomer 1), dihydrochloride salt | 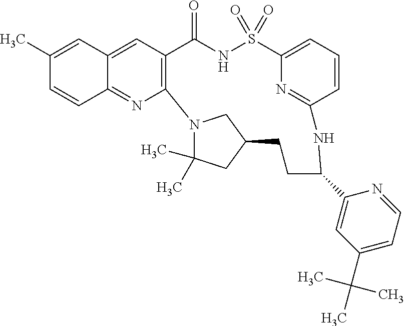 [SMILES: Cc1ccc2nc3c(cc2c1)C(=O)NS(=O)(=O)c1cccc(n1)N[C@H](c1cc(C(C)(C)C)ccn1)CC[C@@H]1CN3C(C)(C)C1] | 1.18 | 612.288 | 613.6 | LC method A |
| Compound 5 (Diastereomer 2), dihydrochloride salt | 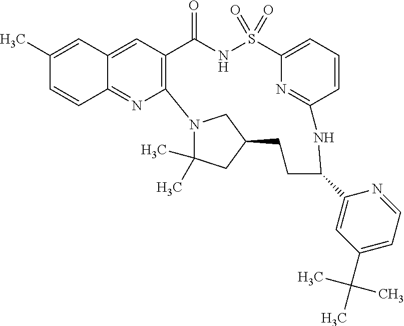 [SMILES: Cc1ccc2nc3c(cc2c1)C(=O)NS(=O)(=O)c1cccc(n1)N[C@H](c1cc(C(C)(C)C)ccn1)CC[C@@H]1CN3C(C)(C)C1] | 1.34 | 612.288 | 613.5 | LC method A |
| Compound 8 (Diastereomer 1), dihydrochloride salt | 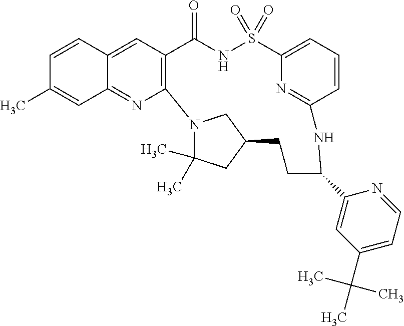 [SMILES: Cc1ccc2cc3c(nc2c1)N1C[C@@H](CC[C@@H](c2cc(C(C)(C)C)ccn2)Nc2cccc(n2)S(=O)(=O)NC3=O)CC1(C)C] | 1.17 | 612.288 | 613.5 | LC method A |
| Compound 7 (Diastereomer 2), dihydrochloride salt | 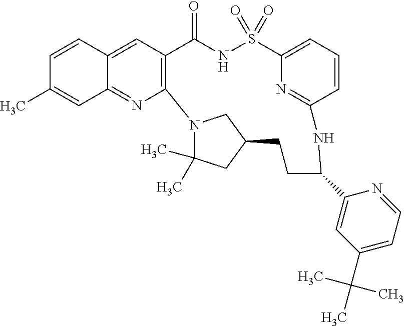 [SMILES: Cc1ccc2cc3c(nc2c1)N1C[C@@H](CC[C@@H](c2cc(C(C)(C)C)ccn2)Nc2cccc(n2)S(=O)(=O)NC3=O)CC1(C)C] | 1.33 | 612.288 | 613.6 | LC method A |
| Compound 10 (Diastereomer 1), dihydrochloride salt | 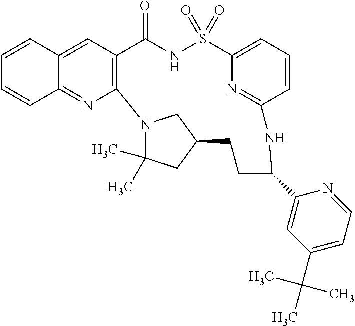 [SMILES: CC(C)(C)c1ccnc([C@@H]2CC[C@@H]3CN(c4nc5ccccc5cc4C(=O)NS(=O)(=O)c4cccc(n4)N2)C(C)(C)C3)c1] | 1.11 | 598.273 | 599.7 | LC method A |
| Compound 9 (Diastereomer 2), dihydrochloride salt | 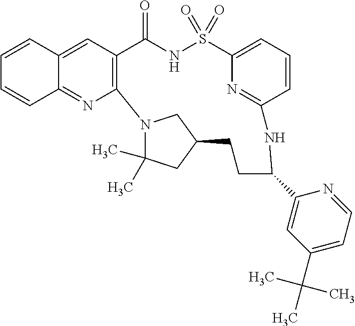 [SMILES: CC(C)(C)c1ccnc([C@@H]2CC[C@@H]3CN(c4nc5ccccc5cc4C(=O)NS(=O)(=O)c4cccc(n4)N2)C(C)(C)C3)c1] | 1.3 | 598.273 | 599.6 | LC method A |

### Example 91: Preparation of (14S)-8-tert-Butyl-12,12,17-trimethyl-17-(pyridin-2-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 45 (diastereomer 1), and Compound 44 (diastereomer 2)

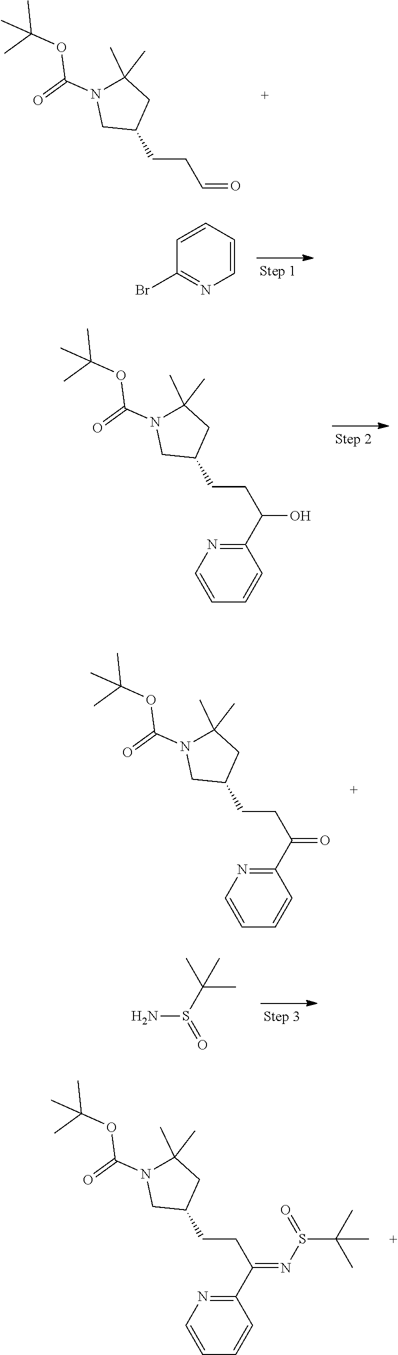

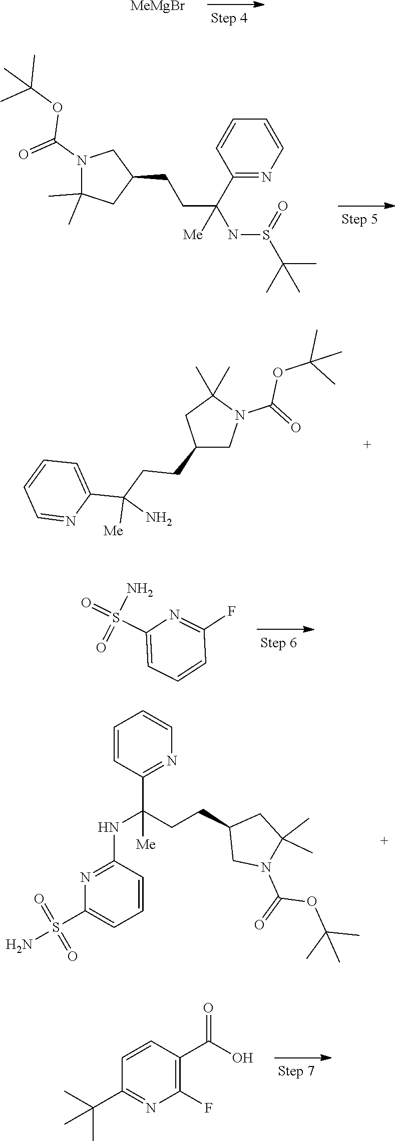

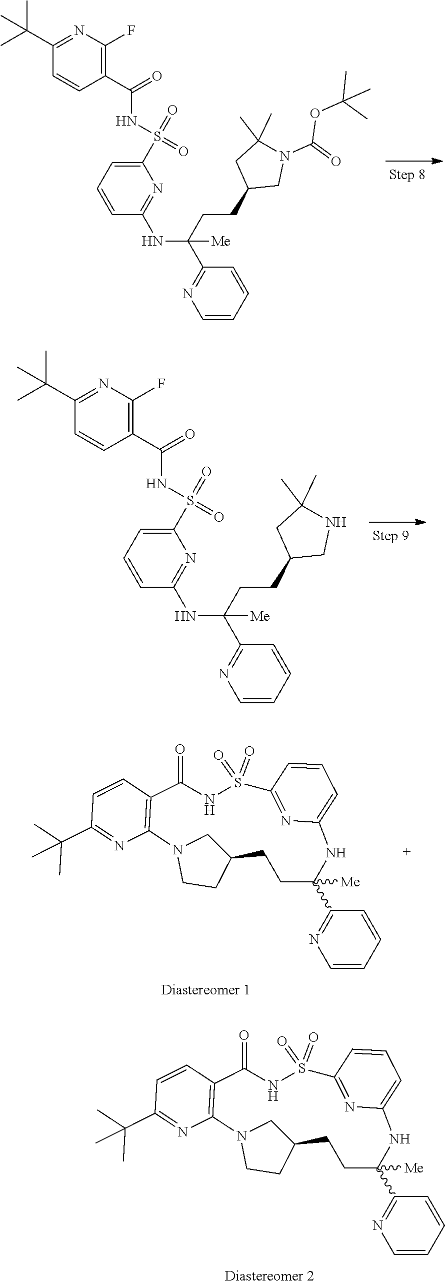

### Step 1: tert-Butyl (4S)-4-[3-hydroxy-3-(2-pyridyl)propyl]-2,2-dimethyl-pyrrolidine-1-carboxylate

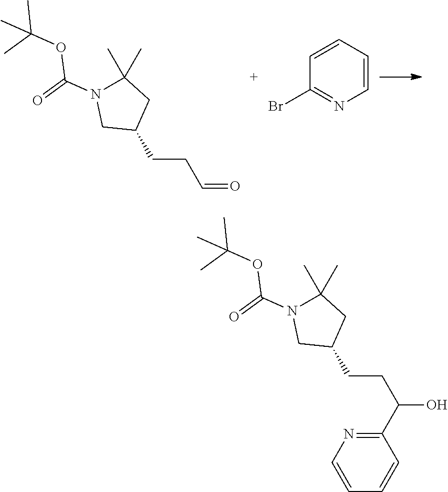

To a mixture of 2-bromopyridine (5.3 mL, 55.584 mmol) in anhdrous THF (100 mL) stirring vigorously at −78° C. under nitrogen was added dropwise n-butyllithium (31.5 mL of 1.8 M, 56.700 mmol) in hexanes. The mixture was stirred vigorously for 0.5 h at −78° C. and a solution of tert-butyl (4S)-2,2-dimethyl-4-(3-oxopropyl)pyrrolidine-1-carboxylate (6.00 g, 22.322 mmol) in THF (70 mL) was added dropwise. The reaction was stirred for 30 min. at −78° C. The reaction was quenched with saturated aqueous NH₄Cl (200 mL) and was allowed to warm to rt.H₂O (200 mL) was added and the mixture was extracted with EtOAc (3×250 mL). The combined organic layers were washed with saturated aqueous NaCl (100 mL), dried over anhydrous sodium sulfate, filtered and concentrated under vacuum to give the product as a brown oil, tert-butyl (4S)-4-[3-hydroxy-3-(2-pyridyl)propyl]-2,2-dimethyl-pyrrolidine-1-carboxylate (10.71 g, 105%) ESI-MS m/z calc. 334.2256, found 335.2 (M+1)⁺; Retention time: 2.28 minutes (LC method B).

### Step 2: tert-Butyl (4S)-2,2-dimethyl-4-[3-oxo-3-(2-pyridyl)propyl]pyrrolidine-1-carboxylate

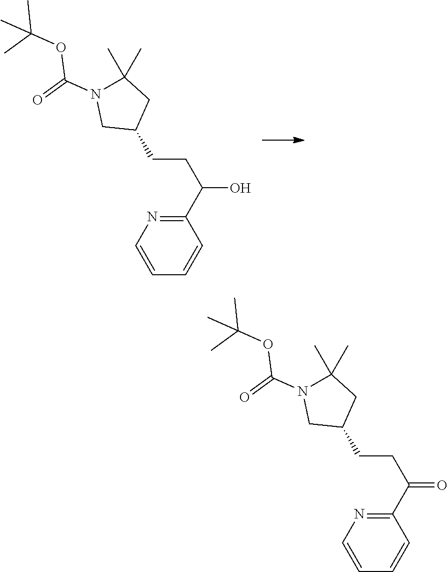 [SMILES: CC(C)(C)OC(=O)N1C[C@@H](CCC(O)c2ccccn2)CC1(C)C ; CC(C)(C)OC(=O)N1C[C@@H](CCC(=O)c2ccccn2)CC1(C)C]

tert-Butyl (4S)-4-[3-hydroxy-3-(2-pyridyl)propyl]-2,2-dimethyl-pyrrolidine-1-carboxylate (10.71 g, 23.472 mmol) was dissolved in DCM (90 mL). The solution was cooled in ice water bath. Dess-Martin periodinane (13.59 g, 30.439 mmol) was added in small portions. The mixture was stirred under nitrogen with ice bath removed. After 3 h warming to rt, LCMS (ELSD) showed disappearance of starting material. A solution of Na₂S₂O₃ (˜10 g) in a saturated aqueous sodium bicarbonate (250 mL) was added. The mixture was stirred for 30 min. The layers were separated. The DCM layer was further extracted with sodium bicarbonate (saturated aqueous 200 mL×2), washed with saturated aqueous NaCl (200 mL) and dried over anhydrous sodium sulfate, filtered and concentrated. The crude brown oil (10.66 g) was subjected to flash chromatography (loaded in DCM) (120 g SiO2, eluting 0 to 30% EtOAc/hexanes in 35 min.). Final Purified Mass=4.38 g (orange oil) tert-butyl (4S)-2,2-dimethyl-4-[3-oxo-3-(2-pyridyl)propyl]pyrrolidine-1-carboxylate (4.38 g, 54%) ESI-MS m/z calc. 332.21, found 333.2 (M+1)⁺; Retention time: 3.05 minutes (LC method B).

### Step 3: tert-Butyl (4S)-4-[(3E)-3-tert-butylsulfinylimino-3-(2-pyridyl)propyl]-2,2-dimethyl-pyrrolidine-1-carboxylate

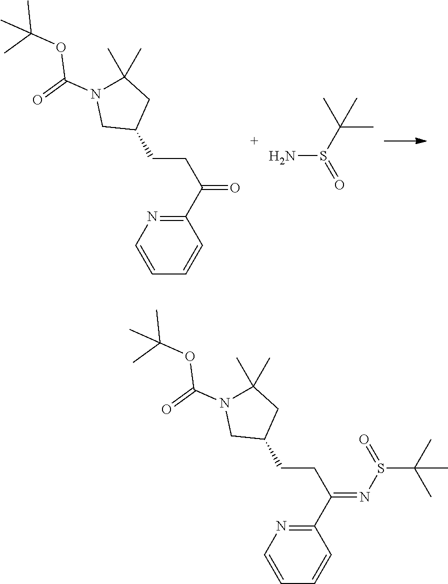

To a solution of tert-butyl (4S)-2,2-dimethyl-4-[3-oxo-3-(2-pyridyl)propyl]pyrrolidine-1-carboxylate (5.6 g, 13.476 mmol) in anhydrous THF (36 mL) stirring at rt under nitrogen was added 2-methylpropane-2-sulfinamide (3.3963 g, 27.181 mmol) and titanium (IV) ethoxide (8.5 mL, 36.589 mmol). The reaction was heated to and stirred at 80° C. for 4 days. The reaction was cooled in an ice-bath, diluted with EtOAc (36 mL), and poured into an ice-cold saturated aqueous NaCl (72 mL) with gentle stirring. The precipitate formed was filtered through a pad of celite and washed with EtOAc (2×36 mL). The filtrate was washed with saturated aqueous NaCl (72 mL), dried over anhydrous sodium sulfate, filtered and concentrated under vacuum. The crude oil (7.41 g) was subjected to flash chromatography (Loaded in DCM) (80 g SiO2, eluting 0 to 30% EtOAc/hexanes in 40 min.) to give, as an orange oil, tert-butyl (4S)-4-[(3E)-3-tert-butylsulfinylimino-3-(2-pyridyl)propyl]-2,2-dimethyl-pyrrolidine-1-carboxylate (3.0615 g, 47%) ESI-MS m/z calc. 435.2556, found 436.5 (M+1)⁺; Retention time: 6.55 minutes (LC method C).

### Step 4: tert-Butyl (4S)-4-[3-(tert-butylsulfinylamino)-3-(2-pyridyl)butyl]-2,2-dimethyl-pyrrolidine-1-carboxylate

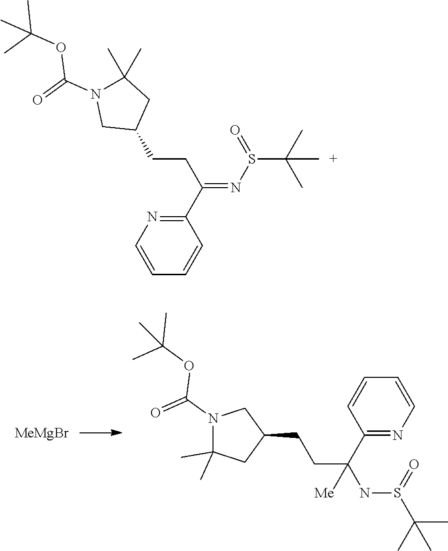

tert-Butyl (4S)-4-[(3E)-3-tert-butylsulfinylimino-3-(2-pyridyl)propyl]-2,2-dimethyl-pyrrolidine-1-carboxylate (2.29 g, 4.7312 mmol) was dissolved in anhydrous THF (16.8 mL) under nitrogen and the solution was cooled and stirred in a dry ice-acetone bath (−78° C.) for 5 min. bromo(methyl)magnesium (9 mL of 1.4 M in THF/toluene (1:3, v:v), 12.600 mmol) was added dropwise and the reaction was warmed in a dry ice-acetonitrile bath (−40-30° C.) and stirred for 1 h. Saturated aqueous NH₄Cl (50 mL) was added and the mixture was allowed to warm up to rt. EtOAc (50 mL) and H₂O (50 mL) were added. The layers were separated and the aqueous layer was extracted with EtOAc (50 mL). The combined EtOAc layers were washed with saturated aqueous NaCl (50 mL), dried over anhydrous sodium sulfate, filtered and concentrated under vacuum. The crude was subjected to flash chromatography (Loaded in DCM) (25 g SiO2, eluting 0 to 100% EtOAc/hexanes in 40 min.). Appropriate fractions were collected and concentrated to give as a light brown foamy solid, tert-butyl (4S)-4-[3-(tert-butylsulfinylamino)-3-(2-pyridyl)butyl]-2,2-dimethyl-pyrrolidine-1-carboxylate (2.1258 g, 97%). ESI-MS m/z calc. 451.2869, found 452.5 (M+1)⁺; Retention time: 5.02 minutes (LC method C).

### Step 5: tert-Butyl (4S)-4-[3-amino-3-(2-pyridyl)butyl]-2,2-dimethyl-pyrrolidine-1-carboxylate

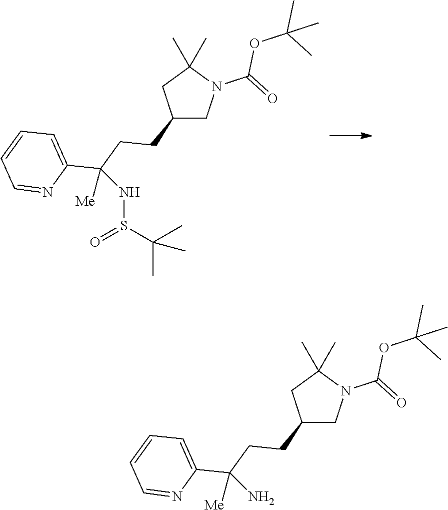 [SMILES: CC(C)(C)OC(=O)N1C[C@@H](CCC(C)(NS(=O)C(C)(C)C)c2ccccn2)CC1(C)C ; CC(C)(C)OC(=O)N1C[C@@H](CCC(C)(N)c2ccccn2)CC1(C)C]

tert-Butyl (4S)-4-[3-(tert-butylsulfinylamino)-3-(2-pyridyl)butyl]-2,2-dimethyl-pyrrolidine-1-carboxylate (2.1258 g, 4.6124 mmol) was dissolved in THF (15 mL) and water (3 mL). Molecular iodine (350.0 mg, 0.0710 mL, 1.3762 mmol) was added. The mixture was stirred at 55° C. for 2 h. It was then cooled to rt and partitioned between EtOAc (35 mL) and Na₂S₂O₃ (7 g) in saturated aqueous sodium bicarbonate (35 mL). The layers were separated and the aqueous layer was extracted once with EtOAc (50 mL). The organic layer was washed with saturated aqueous NaCl (50 mL), dried over anhydrous sodium sulfate, filtered and concentrated under vacuum to give tert-butyl (4S)-4-[3-amino-3-(2-pyridyl)butyl]-2,2-dimethyl-pyrrolidine-1-carboxylate (1.8514 g, 104%) ESI-MS m/z calc. 347.2573, found 348.3 (M+1)⁺; Retention time: 2.46 minutes (LC method B).

### Step 6: tert-Butyl (4S)-2,2-dimethyl-4-[3-(2-pyridyl)-3-[(6-sulfamoyl-2-pyridyl)amino]butyl]pyrrolidine-1-carboxylate

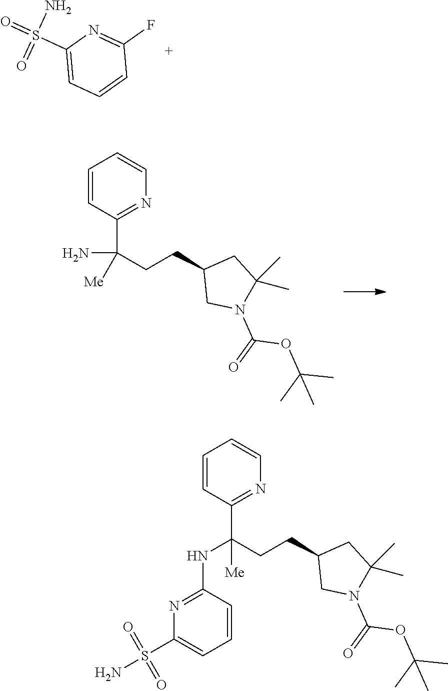 [SMILES: NS(=O)(=O)c1cccc(F)n1 ; CC(C)(C)OC(=O)N1C[C@@H](CCC(C)(N)c2ccccn2)CC1(C)C ; CC(C)(C)OC(=O)N1C[C@@H](CCC(C)(Nc2cccc(S(N)(=O)=O)n2)c2ccccn2)CC1(C)C]

To a mixture of tert-butyl (4S)-4-[3-amino-3-(2-pyridyl)butyl]-2,2-dimethyl-pyrrolidine-1-carboxylate (1.245 g, 3.2245 mmol) and 6-fluoropyridine-2-sulfonamide (2 g, 10.785 mmol) in DMSO (5 mL) was added DIEA (1.4840 g, 2 mL, 11.482 mmol). The mixture was stirred at 125° C. for 6 days. The reaction mixture was cooled to rt. and then diluted with water (200 mL) and EtOAc (100 mL). The layers were separated and the organic layer was washed with brine (2×100 mL), dried over anhydrous Na₂SO₄, and concentrated. The residue was purified by silica gel chromatography (120 g column), using 0-80% EtOAc in hexanes to afford tert-butyl (4S)-2,2-dimethyl-4-[3-(2-pyridyl)-3-[(6-sulfamoyl-2-pyridyl)amino]butyl]pyrrolidine-1-carboxylate (516.3 mg, 30%) as a light brown solid. ¹H NMR (500 MHz, DMSO-d₆) δ 8.56-8.51 (m, 1H), 7.77-7.70 (m, 1H), 7.56-7.48 (m, 2H), 7.41-7.36 (m, 1H), 7.26-7.20 (m, 1H), 6.97-6.93 (m, 1H), 6.74-6.61 (m, 3H), 3.50-3.40 (m, 1H), 2.67-2.57 (m, 1H), 2.45-2.26 (m, 1H), 2.17-2.01 (m, 1H), 1.96-1.87 (m, 1H), 1.86-1.75 (m, 1H), 1.74-1.70 (m, 3H), 1.40-1.34 (m, 9H), 1.31-1.23 (m, 4H), 1.18-1.16 (m, 3H), 1.14-1.06 (m, 1H), 1.06-0.97 (m, 1H). ESI-MS m/z calc. 503.2566, found 504.6 (M+1)⁺; Retention time: 4.44 minutes (LC method C).

### Step 7: tert-Butyl (4S)-4-[3-[[6-[(6-tert-butyl-2-fluoro-pyridine-3-carbonyl)sulfamoyl]-2-pyridyl]amino]-3-(2-pyridyl)butyl]-2,2-dimethyl-pyrrolidine-1-carboxylate

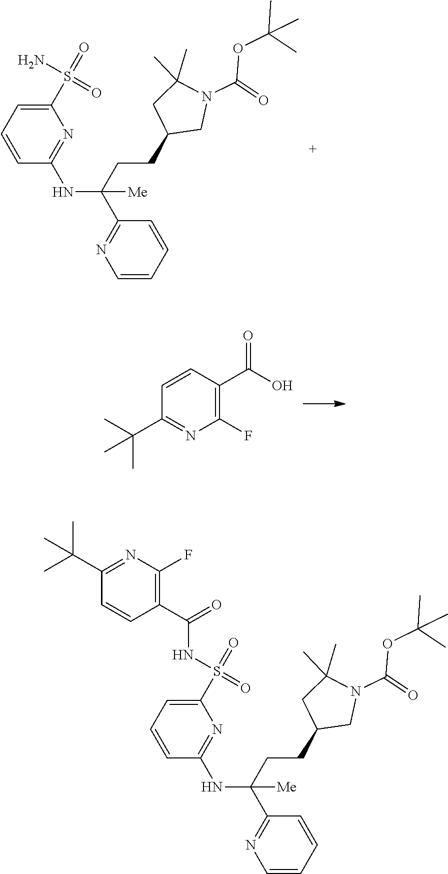

To a solution of 6-tert-butyl-2-fluoro-pyridine-3-carboxylic acid (580 mg, 2.7940 mmol) in THF (2 mL) was added CDI (490 mg, 3.0219 mmol) and the mixture was stirred at rt for 16 h. Then tert-butyl (4S)-2,2-dimethyl-4-[3-(2-pyridyl)-3-[(6-sulfamoyl-2-pyridyl)amino]butyl]pyrrolidine-1-carboxylate (405 mg, 0.6929 mmol) was added followed by DBU (407.20 mg, 0.4 mL, 2.6748 mmol) and the resulting mixture was stirred for 2 h. The reaction was quenched with 1:1 mixture of saturated ammonium chloride and brine solutions, then extracted with ethyl acetate. The combined organic layers washed brine, dried over sodium sulfate, filtered and evaporated. The resultant residue was purified by flash chromatography using 20-70% EtOAc in Hexane to provide tert-butyl (4S)-4-[3-[[6-[(6-tert-butyl-2-fluoro-pyridine-3-carbonyl)sulfamoyl]-2-pyridyl]amino]-3-(2-pyridyl)butyl]-2,2-dimethyl-pyrrolidine-1-carboxylate (400 mg, 80%) as light yellow solid. ESI-MS m/z calc. 682.3313, found 683.7 (M+1)⁺; Retention time: 5.6 minutes (LC method C).

### Step 8: 6-tert-Butyl-N-[[6-[[3-[(3S)-5,5-dimethylpyrrolidin-3-yl]-1-methyl-1-(2-pyridyl)propyl]amino]-2-pyridyl]sulfonyl]-2-fluoro-pyridine-3-carboxamide

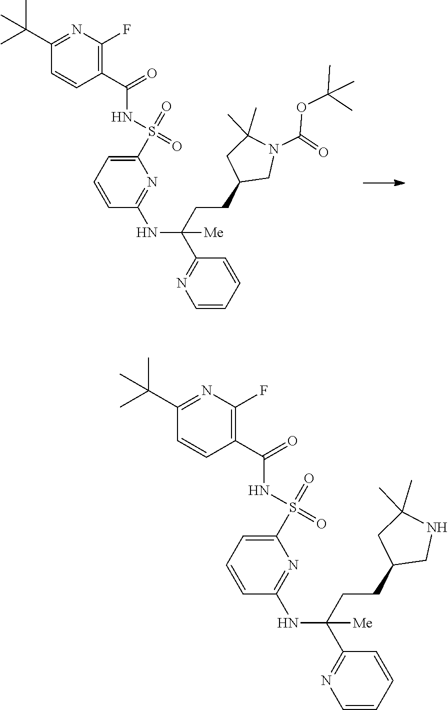

To a solution of tert-butyl (4S)-4-[3-[[6-[(6-tert-butyl-2-fluoro-pyridine-3-carbonyl)sulfamoyl]-2-pyridyl]amino]-3-(2-pyridyl)butyl]-2,2-dimethyl-pyrrolidine-1-carboxylate (265 mg, 0.3498 mmol) in DCM (1.5 mL) was added TFA (740.00 mg, 0.5 mL, 6.4899 mmol) and the mixture was stirred at rt for 1 h. The solvent was evaporated. The residue was dissolved in EtOAc (10 mL). The mixture was extracted with H₂O (5×20 mL). The aqueous was basified with solid sodium bicarbonate and extracted with DCM (2×50 mL). The organic layer was dried and evaporated to provide 6-tert-butyl-N-[[6-[[3-[(3S)-5,5-dimethylpyrrolidin-3-yl]-1-methyl-1-(2-pyridyl)propyl]amino]-2-pyridyl]sulfonyl]-2-fluoro-pyridine-3-carboxamide (153 mg, 74%) as off white solid. ESI-MS m/z calc. 582.2788, found 583.6 (M+1)⁺; Retention time: 2.0 minutes (LC method B).

### Step 9: (14S)-8-tert-Butyl-12,12,17-trimethyl-17-(pyridin-2-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 45 (diastereomer 1), and (14S)-8-tert-butyl-12,12,17-trimethyl-17-(pyridin-2-yl)-2λ⁶-thia-3,9,11,18,23-pentaazatetracyclo[17.3.1.111,14.05,10]tetracosa-1(23),5(10),6,8,19,21-hexaene-2,2,4-trione, Compound 44 (diastereomer 2)

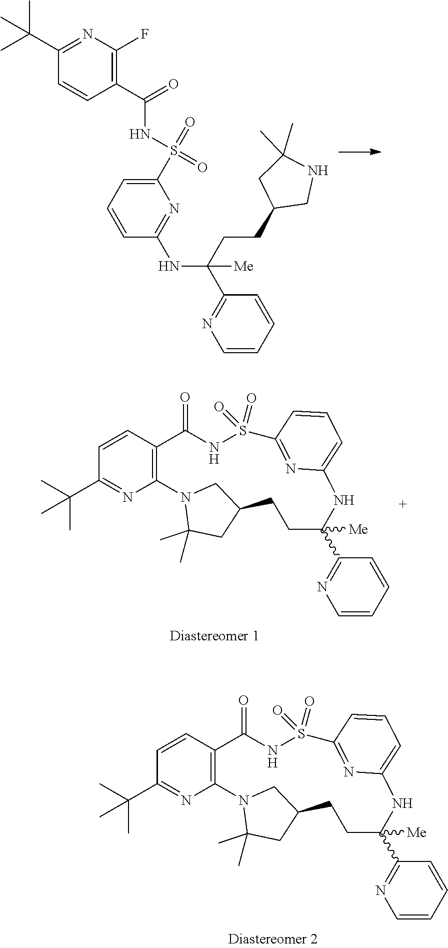

To a solution of 6-tert-butyl-N-[[6-[[3-[(3S)-5,5-dimethylpyrrolidin-3-yl]-1-methyl-1-(2-pyridyl)propyl]amino]-2-pyridyl]sulfonyl]-2-fluoro-pyridine-3-carboxamide (306 mg, 0.5207 mmol) in DMF (3 mL) was added K₂CO₃ (250 mg, 1.8089 mmol). The mixture was purged with nitrogen for 5 min. The mixture was heated at 140° C. for 16 h and cooled down to rt. The reaction was purified by reverse phase HPLC (20 to 80% acetonitrile in water over 20 min, flow rate 40 mL/min 5 mM HCl as a modifier), to provide two isomers:

Diastereomer 1: (14S)-8-tert-butyl-12,12,17-trimethyl-17-(pyridin-2-yl)-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (59.1 mg, 18%) as a white solid. ¹H NMR (500 MHz, DMSO-d₆) δ 12.78 (s, 1H), 8.68-8.57 (m, 1H), 7.96 (s, 1H), 7.87-7.72 (m, 2H), 7.69 (t, J=7.7, 7.7 Hz, 1H), 7.38-7.32 (m, 2H), 7.13 (d, J=8.5 Hz, 1H), 6.60 (d, J 7.9 Hz, 1H), 3.47-3.21 (m, 2H), 2.18-2.10 (m, 1H), 2.00-1.84 (m, 1H), 1.80-1.60 (m, 5H), 1.57-1.40 (m, 6H), 1.30-1.22 (m, 10H), 1.07-0.70 (m, 2H). ESI-MS m/z calc. 562.2726, found 563.0 (M+1)⁺; Retention time: 2.39 minutes (LC method B).

Diastereomer 2: (14S)-8-tert-butyl-12,12,17-trimethyl-17-(pyridin-2-yl)-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (55.4 mg, 17%) as a white solid. ¹H NMR (500 MHz, DMSO-d₆) δ 12.58 (s, 1H), 8.60 (s, 1H), 7.73 (m, 4H), 7.34 (d, J=7.3 Hz, 1H), 7.29 (d, J=7.8 Hz, 1H), 7.10 (d, J=8.5 Hz, 1H), 6.56 (d, J=7.8 Hz, 1H), 3.28 (s, 1H), 3.03 (s, 1H), 2.27 (s, 3H), 1.85 (d, J=9.9 Hz, 2H), 1.55 (m, 6H), 1.48 (s, 3H), 1.45-1.37 (m, 1H), 1.25 (s, 9H), 0.98 (q, J=11.8 Hz, 1H). ESI-MS m/z calc. 562.2726, found 563.0 (M+1)⁺; Retention time: 2.29 minutes (LC method B).

### Example 92: Synthesis of tert-butyl 3-oxo-2,3-dihydro-1H-pyrazole-1-carboxylate

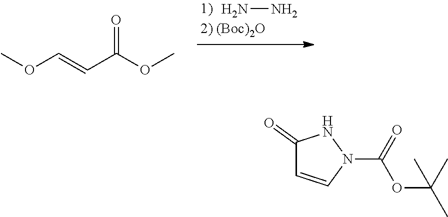

A 50 L reactor was started, and the jacket was set to 20° C., with stirring at 150 rpm, reflux condenser (10° C.) and nitrogen purge. MeOH (2.860 L) and methyl (E)-3-methoxyprop-2-enoate (2.643 kg, 22.76 mol) were added, and the reactor was capped. The reaction was heated to an internal temperature of 40° C., and the system was set to hold jacket temperature at 40° C. Hydrazine hydrate (1300 g of 55% w/w, 22.31 mol) was added portion wise via addition funnel over 30 min. The reaction was heated to 60° C. for 1 h. The reaction mixture was cooled to 20° C. and triethylamine (2.483 kg, 3.420 L, 24.54 mol) was added portion-wise, maintaining reaction temperature <30° C. A solution of Boc anhydride (di-tert-butyl dicarbonate) (4.967 kg, 5.228 L, 22.76 mol) in MeOH (2.860 L) was added portion-wise maintaining temperature <45° C. The reaction mixture was stirred at 20° C. for 16 h. The reaction solution was partially concentrated to remove MeOH, resulting in a clear, light amber oil. The resulting oil was transferred to the 50 L reactor, stirred and water (7.150 L) and heptane (7.150 L) were added. The additions caused a small amount of the product to precipitate. The aqueous layer was drained into a clean container, and the interface and heptane layer were filtered to separate the solid (product). The aqueous layer was transferred back to the reactor, and the collected solid was placed back into the reactor and mixed with the aqueous layer. A dropping funnel was added to the reactor and loaded with acetic acid (1.474 kg, 1.396 L, 24.54 mol) and added dropwise. The jacket was set to 0° C. to absorb the quench exotherm. After the addition was complete (pH=5), the reaction mixture was stirred for 1 h. The solid was collected by filtration and washed with water (7.150 L), and washed a second time with water (3.575 L). The crystalline solid was transferred into a 20 L rotovap bulb, and heptane (7.150 L) was added. The mixture was slurried at 45° C. for 30 min, and 1-2 volumes of solvent were distilled off. The slurry in the rotovap flask was filtered, and the solids were washed with heptane (3.575 L). The solid was further dried in vacuo (50° C., 15 mbar) to give tert-butyl 5-oxo-1H-pyrazole-2-carboxylate (2921 g, 71%) as a coarse, crystalline solid. ¹H NMR (400 MHz, DMSO-d₆) δ 10.95 (s, 1H), 7.98 (d, J=2.9 Hz, 1H), 5.90 (d, J=2.9 Hz, 1H), 1.54 (s, 9H).

### Example 93: Preparation of 2-Chloro-6-[3-[2-[1-(trifluoromethyl)cyclopropyl]ethoxy]pyrazol-1-yl]pyridine-3-carboxylic acid

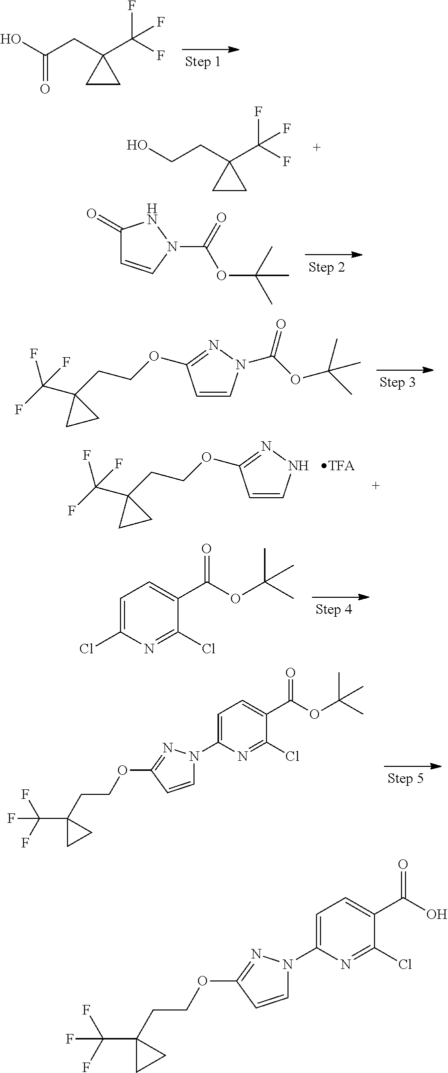

### Step 1: 2-[1-(Trifluoromethyl)cyclopropyl]ethanol

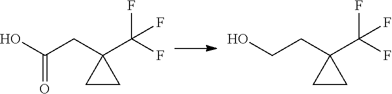 [SMILES: O=C(O)CC1(C(F)(F)F)CC1 ; OCCC1(C(F)(F)F)CC1]

To a solution of lithium aluminum hydride (293 mg, 7.732 mmol) in tetrahydrofuran (10.00 mL) in an ice-bath, 2-[1-(trifluoromethyl)cyclopropyl]acetic acid (1.002 g, 5.948 mmol) in tetrahydrofuran (3.0 mL) was added dropwise over a period of 30 min keeping the reaction temperature below 20° C. The mixture was allowed to gradually warm to ambient temperature and was stirred for 18 h. The mixture was cooled with an ice-bath and sequentially quenched with water (295 μL, 16.36 mmol), aqueous sodium hydroxide (297 μL of 6 M, 1.784 mmol), and then water (884 μL, 49.07 mmol) to afford a granular solid in the mixture. The solid was filtered off using celite, and the precipitate was washed with diethyl ether. The filtrate was further dried with magnesium sulfate and filtered and concentrated in vacuo to afford the product with residual tetrahydrofuran and diethyl ether. The mixture was taken directly into the next step without further purification.

### Step 2: tert-Butyl 3-[2-[1-(trifluoromethyl)cyclopropyl]ethoxy]pyrazole-1-carboxylate

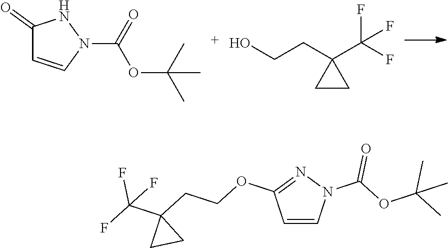 [SMILES: CC(C)(C)OC(=O)n1ccc(=O)[nH]1 ; OCCC1(C(F)(F)F)CC1 ; CC(C)(C)OC(=O)n1ccc(OCCC2(C(F)(F)F)CC2)n1]

tert-Butyl 5-oxo-1H-pyrazole-2-carboxylate (1.043 g, 5.660 mmol), 2-[1-(trifluoromethyl)cyclopropyl]ethanol (916 mg, 5.943 mmol), and triphenylphosphine (1.637 g, 6.243 mmol) were combined in tetrahydrofuran (10.48 mL) and the reaction was cooled in an ice-bath. Diisopropyl azodicarboxylate (1.288 g, 1.254 mL, 6.368 mmol) was added dropwise to the reaction mixture, and the reaction was allowed to warm to room temperature for 16 h. The mixture was evaporated, and the resulting material was partitioned between ethyl acetate (30 mL) and 1N sodium hydroxide (30 mL). The organic layer was separated, washed with brine (30 mL), dried over sodium sulfate, and concentrated. The crude material was purified by silica gel chromatography eluting with a gradient of ethyl acetate in hexanes (0-30%) to give tert-butyl 3-[2-[1-(trifluoromethyl)cyclopropyl]ethoxy]pyrazole-1-carboxylate (1.03 g, 57%). ESI-MS m/z calc. 320.13, found 321.1 (M+1)+; Retention time: 0.72 min (LC method J).

### Step 3: 3-[2-[1-(Trifluoromethyl)cyclopropyl]ethoxy]-1H-pyrazole

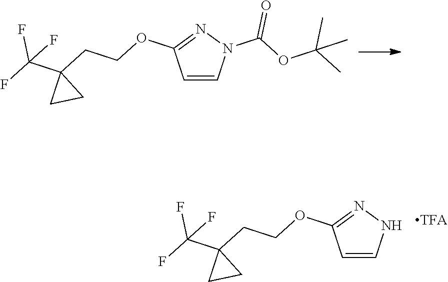 [SMILES: CC(C)(C)OC(=O)n1ccc(OCCC2(C(F)(F)F)CC2)n1 ; FC(F)(F)C1(CCOc2cc[nH]n2)CC1]

tert-Butyl-3-[2-[1-(trifluoromethyl)cyclopropyl]ethoxy]pyrazole-1-carboxylate (1.03 g, 3.216 mmol) was dissolved in dichloromethane (10.30 mL) with trifluoroacetic acid (2.478 mL, 32.16 mmol), and the reaction was stirred at room temperature for 2 h. The reaction was evaporated, and the resulting oil was partitioned between ethyl acetate (10 mL) and a saturated sodium bicarbonate solution. The organic layer was separated, washed with brine, dried over sodium sulfate, and evaporated to give 3-[2-[1-(trifluoromethyl)cyclopropyl]ethoxy]-1H-pyrazole (612 mg, 86%). ¹H NMR (400 MHz, dimethyl sulfoxide-d₆) δ 11.86 (s, 1H), 7.50 (t, J=2.1 Hz, 1H), 5.63 (t, J=2.3 Hz, 1H), 4.14 (t, J=7.1 Hz, 2H), 2.01 (t, J=7.1 Hz, 2H), 0.96-0.88 (m, 2H), 0.88-0.81 (m, 2H). ESI-MS m/z calc. 220.08, found 221.0 (M+1)⁺; Retention time: 0.5 min (LC method J).

### Step 4: tert-Butyl 2-chloro-6-[3-[2-[1-(trifluoromethyl)cyclopropyl]ethoxy]pyrazol-1-yl]pyridine-3-carboxylate

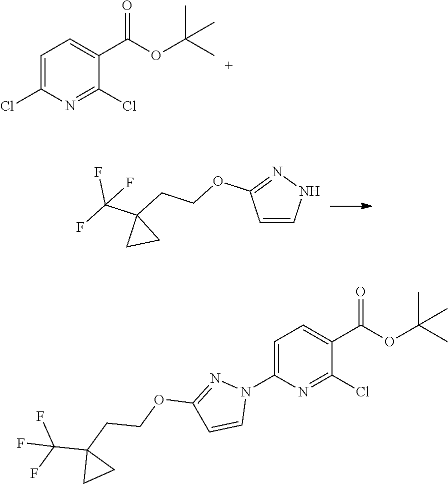 [SMILES: CC(C)(C)OC(=O)c1ccc(Cl)nc1Cl ; FC(F)(F)C1(CCOc2cc[nH]n2)CC1 ; CC(C)(C)OC(=O)c1ccc(-n2ccc(OCCC3(C(F)(F)F)CC3)n2)nc1Cl]

tert-Butyl 2,6-dichloropyridine-3-carboxylate (687 mg, 2.770 mmol), 3-[2-[1-(trifluoromethyl)cyclopropyl]ethoxy]-1H-pyrazole (610 mg, 2.770 mmol), and freshly ground potassium carbonate (459 mg, 3.324 mmol) were combined in anhydrous dimethyl sulfoxide (13.75 mL). 1,4-diazabicyclo[2.2.2]octane (62 mg, 0.554 mmol) was added, and the mixture was stirred at room temperature under nitrogen for 16 h. The reaction mixture was diluted with water (20 mL) and stirred for 15 min. The resulting solid was collected and washed with water. The solid was dissolved in dichloromethane and dried over magnesium sulfate. The mixture was filtered and concentrated to give tert-butyl 2-chloro-6-[3-[2-[1-(trifluoromethyl)cyclopropyl]ethoxy]pyrazol-1-yl]pyridine-3-carboxylate (1.01 g, 84%). ESI-MS m/z calc. 431.12, found 432.1 (M+1)⁺; Retention time: 0.88 min (LC method J).

### Step 5: 2-Chloro-6-[3-[2-[1-(trifluoromethyl)cyclopropyl]ethoxy]pyrazol-1-yl]pyridine-3-carboxylic acid

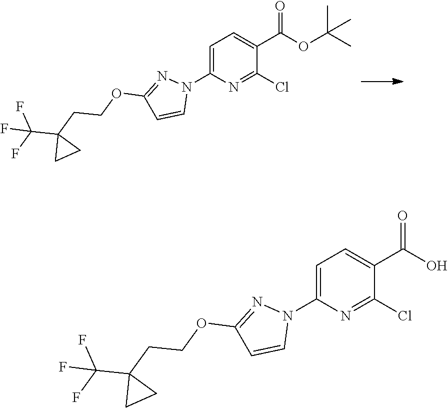 [SMILES: CC(C)(C)OC(=O)c1ccc(-n2ccc(OCCC3(C(F)(F)F)CC3)n2)nc1Cl ; O=C(O)c1ccc(-n2ccc(OCCC3(C(F)(F)F)CC3)n2)nc1Cl]

tert-Butyl 2-chloro-6-[3-[2-[1-(trifluoromethyl)cyclopropyl]ethoxy]pyrazol-1-yl]pyridine-3-carboxylate (1.01 g, 2.339 mmol) and trifluoroacetic acid (1.8 mL, 23.39 mmol) were combined in dichloromethane (10 mL) and heated at 40° C. for 3 h. The reaction was concentrated. Hexanes were added, and the mixture was concentrated again to give 2-chloro-6-[3-[2-[1-(trifluoromethyl)cyclopropyl]ethoxy]pyrazol-1-yl]pyridine-3-carboxylic acid (873 mg, 99%) ESI-MS m/z calc. 375.06, found 376.1 (M+1)⁺; Retention time: 0.69 min (LC method J).

### Example 94: Preparation of 2-Chloro-6-[3-(2-dispiro[2.0.2.1]heptan-7-ylethoxy)pyrazol-1-yl]pyridine-3-carboxylic acid

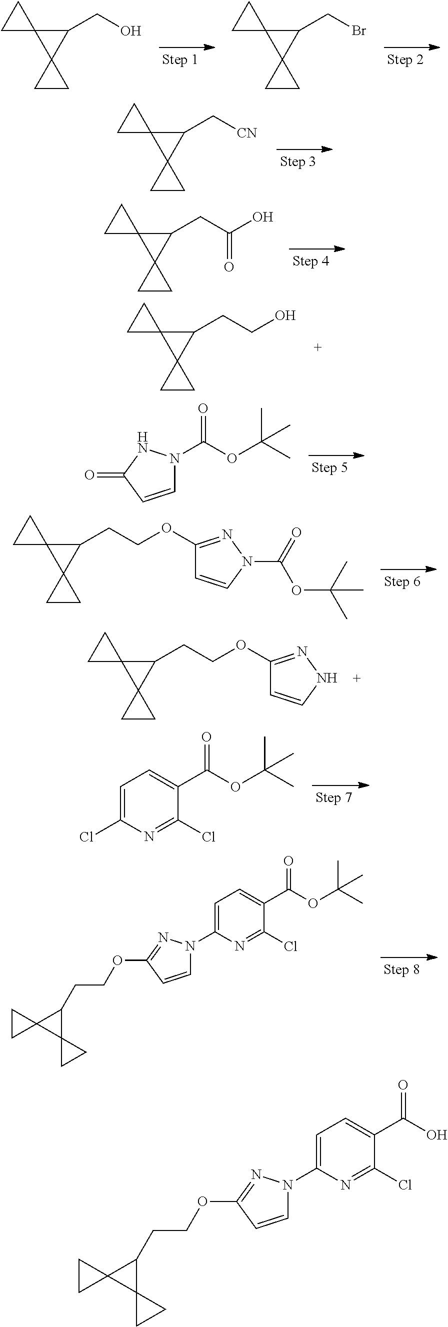

### Step 1: 7-(Bromomethyl)dispiro[2.0.2.1]heptane

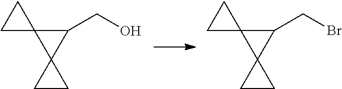 [SMILES: OCC1C2(CC2)C12CC2 ; BrCC1C2(CC2)C12CC2]

A 1000 mL, 3-neck round bottom flask was fitted with a mechanical stirrer, a cooling bath, an addition funnel, a J-Kem temperature probe and a nitrogen inlet/outlet. The vessel was charged under a nitrogen atmosphere with triphenylphosphine (102.7 mL, 443.2 mmol) and dichloromethane (1 L) which provided a clear colorless solution. Stirring was commenced and the cooling bath was charged with acetone. Dry ice was added in portions to the cooling bath until a pot temperature of −15° C. was obtained. The addition funnel was charged with a solution of bromine (22.82 mL, 443.0 mmol) in dichloromethane (220 mL, 10 mL/g) which was subsequently added dropwise over 1 h. Dry ice was added in portions to the cooling bath during the addition to maintain the pot temperature at −15° C. After the addition of bromine was completed, the pale yellow suspension was continued to stir at −15° C. for 15 min at which point the suspension was cooled to −30° C. The addition funnel was charged with a solution of dispiro[2.0.2.1]heptan-7-yl methanol (50 g, 402.6 mmol), pyridine (35.82 mL, 442.9 mmol) and dichloromethane (250 mL, 5 mL/g). The clear pale yellow solution was then added dropwise over 1.5 h maintaining the pot temperature at −30° C. The resulting clear light yellow reaction mixture was allowed to gradually warm to a pot temperature of −5° C. and then continued to stir at −5° C. for 1 h. The reaction mixture then was poured into hexane (2000 mL) which resulted in the formation of a precipitate. The suspension was stirred at room temperature for 30 min and then filtered through a glass frit Buchner funnel with a 20 mm layer of celite. The clear filtrate was concentrated under reduced pressure (water bath temperature at 20° C.) to provide a yellow oil with some precipitate present. The oil was diluted with some hexane, allowed to stand at room temperature for 15 min and then filtered through a glass frit Buchner funnel with a 20 mm layer of celite. The clear filtrate was concentrated under reduced pressure (water bath temperature at 20° C.) to provide 7-(bromomethyl)dispiro[2.0.2.1]heptane (70 g, 93%) as a clear yellow oil. ¹H NMR (400 MHz, Chloroform-d) δ 3.49 (d, J=7.5 Hz, 2H), 1.90 (t, J=7.5 Hz, 1H), 1.06-0.84 (m, 4H), 0.71 (ddd, J=9.1, 5.1, 4.0 Hz, 2H), 0.54 (dddd, J=8.6, 4.8, 3.8, 1.0 Hz, 2H).

### Step 2: 2-Dispiro[2.0.2.1]heptan-7-ylacetonitrile

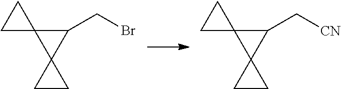 [SMILES: BrCC1C2(CC2)C12CC2 ; N#CCC1C2(CC2)C12CC2]

A 1000 mL, 3-neck round bottom flask was fitted with a mechanical stirrer, a cooling bath used as secondary containment, a J-Kem temperature probe and a nitrogen inlet/outlet. The vessel was charged under a nitrogen atmosphere with 7-(bromomethyl)dispiro[2.0.2.1]heptane (35 g, 187.1 mmol) and dimethyl sulfoxide (245 mL) which provided a clear amber solution. Stirring was commenced and the pot temperature was recorded at 19° C. The vessel was then charged with sodium cyanide (11.46 g, 233.8 mmol) added as a solid in one portion which resulted in a dark solution and a gradual exotherm to 49° C. over 15 min. After a few min the pot temperature began to decrease and the mixture was continued to stir at room temperature overnight (about 15 h). The dark reaction mixture was quenched with ice cold saturated sodium carbonate solution (500 mL) and then transferred to a separatory funnel and partitioned with diethyl ether (500 mL). The organic was removed and the residual aqueous was extracted with diethyl ether (2×250 mL). The combined organics were washed with water (500 mL), dried over sodium sulfate (200 g) and then filtered through a glass frit Buchner funnel. The clear amber filtrate was concentrated under reduced pressure (water bath temperature 20° C.) to provide 2-dispiro[2.0.2.1]heptan-7-ylacetonitrile (21 g, 84%) as a clear dark amber oil. ¹H NMR (400 MHz, Chloroform-d) δ 2.42 (d, J=6.6 Hz, 2H), 1.69 (t, J=6.6 Hz, 1H), 1.02-0.88 (m, 4H), 0.79-0.70 (m, 2H), 0.66-0.55 (m, 2H).

### Step 3: 2-Dispiro[2.0.2.1]heptan-7-ylacetic acid

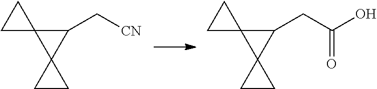 [SMILES: N#CCC1C2(CC2)C12CC2 ; O=C(O)CC1C2(CC2)C12CC2]

To a solution of 2-dispiro[2.0.2.1]heptan-7-ylacetonitrile (2.1 g, 14.19 mmol) in EtOH (32 mL) was added sodium hydroxide (5.12 g, 128.0 mmol) followed by water (13 mL) and the resulting solution was stirred and heated to 70° C. overnight. The mixture was then cooled to room temperature, diluted with water and extracted with diethyl ether. The aqueous phase was adjusted to pH=1 by the addition of 6 N hydrochloric acid (resulting in a cloudy precipitate) and extracted with diethyl ether (3×). The organic phases were dried (magnesium sulfate), filtered and concentrated giving 2-dispiro[2.0.2.1]heptan-7-ylacetic acid (2.19 g, 99% yield, 98% purity) as an orange solid which was used in the next step without further purification. ¹H NMR (400 MHz, Chloroform-d) δ 2.44 (d, J 6.9 Hz, 2H), 1.67 (t, J=6.9 Hz, 1H), 0.91 (ddd, J=9.0, 5.2, 3.9 Hz, 2H), 0.81 (dddd, J 8.9, 5.2, 3.9, 0.5 Hz, 2H), 0.69 (ddd, J=8.9, 5.2, 3.9 Hz, 2H), 0.56-0.44 (m, 2H).

### Step 4: 2-Dispiro[2.0.2.1]heptan-7-ylethanol

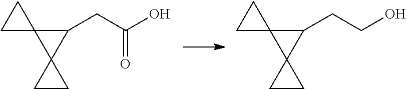 [SMILES: O=C(O)CC1C2(CC2)C12CC2 ; OCCC1C2(CC2)C12CC2]

To lithium aluminum hydride (827.4 mg, 902.3 μL, 21.80 mmol) dissolved in tetrahydrofuran (33.71 mL) cooled in an ice/water bath was added 2-dispiro[2.0.2.1]heptan-7-ylacetic acid (2.552 g, 16.77 mmol) in tetrahydrofuran (7.470 mL) dropwise over 15 min keeping the reaction temperature <20° C. The mixture was allowed to stir a total of 18 h, gradually warming to ambient temperature. The mixture was cooled with an ice/water bath and sequentially quenched with slow addition of water (838.4 mg, 838.4 μL, 46.54 mmol), followed by sodium hydroxide (1.006 mL of 5 M, 5.031 mmol), then water (2.493 g, 2.493 mL, 138.4 mmol) affording a white, granular slurry which was filtered over celite. Washed the filtered solid with diethyl ether. The filtrate was concentrated in vacuo at 300 mbar and 30° C. water bath. Diluted the residue with diethyl ether, dried (magnesium sulfate), filtered and concentrated in vacuo at 300 mbar and 30° C. water bath followed by 30 seconds under vacuum to give 2-dispiro[2.0.2.1]heptan-7-ylethanol (2.318 g, 100%) which was used directly in the ensuing step without further purification. ¹H NMR (400 MHz, Chloroform-d) δ 3.64 (s, 2H), 1.68 (d, J=6.7 Hz, 2H), 1.39 (s, 1H), 1.31 (s, 1H), 0.82 (d, J=14.0 Hz, 4H), 0.65 (s, 2H), 0.50 (d, J=3.6 Hz, 2H).

### Step 5: tert-Butyl 3-(2-dispiro[2.0.2.1]heptan-7-ylethoxy)pyrazole-1-carboxylate

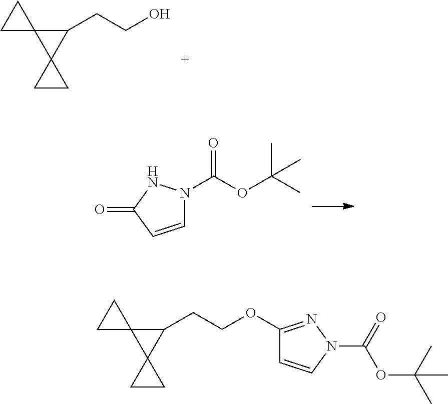 [SMILES: OCCC1C2(CC2)C12CC2 ; CC(C)(C)OC(=O)n1ccc(=O)[nH]1 ; CC(C)(C)OC(=O)n1ccc(OCCC2C3(CC3)C23CC3)n1]

To a solution of tert-butyl 5-oxo-1H-pyrazole-2-carboxylate (2.942 g, 15.97 mmol) and 2-dispiro[2.0.2.1]heptan-7-ylethanol (2.318 g, 16.77 mmol) in tetrahydrofuran (36.78 mL) was added triphenylphosphine (4.399 g, 16.77 mmol). To the mixture was slowly added diisopropyl azodicarboxylate (3.391 g, 3.302 mL, 16.77 mmol) dropwise over 10 min (mild exotherm noted). The reaction mixture was stirred at room temperature for 30 min then at 50° C. for 30 min. The tetrahydrofuran was removed in vacuo. To the crude residue was added toluene (23.54 mL) and the mixture was stirred overnight as a precipitate gradually crystallized. Slurried with Celite then the precipitate was filtered off and washed with toluene (8.705 mL) and again with toluene (8.705 mL). The filtrate was concentrated in vacuo. The crude product was purified by silica gel chromatography using a shallow gradient from 100% hexanes to 100% ethyl acetate giving tert-butyl 3-(2-dispiro[2.0.2.1]heptan-7-ylethoxy)pyrazole-1-carboxylate (3.449 g, 71%). ESI-MS m/z calc. 304.17868, found 305.1 (M+1)⁺; Retention time: 0.82 min (LC method J).

### Step 6: 3-(2-Dispiro[2.0.2.1]heptan-7-ylethoxy)-1H-pyrazole

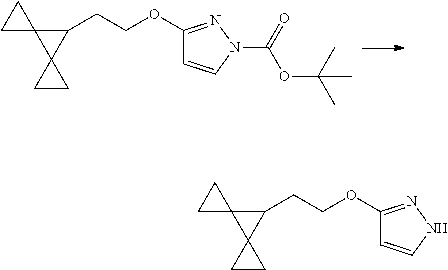 [SMILES: CC(C)(C)OC(=O)n1ccc(OCCC2C3(CC3)C23CC3)n1 ; c1cc(OCCC2C3(CC3)C23CC3)n[nH]1]

tert-Butyl 3-(2-dispiro[2.0.2.1]heptan-7-ylethoxy)pyrazole-1-carboxylate (5.304 g, 17.43 mmol) was dissolved in dichloromethane (53.04 mL) with trifluoroacetic acid (29.81 g, 20.14 mL, 261.4 mmol) and the reaction was stirred at room temperature for 120 min. The reaction was evaporated and the resulting oil was partitioned between ethyl acetate and a saturated sodium bicarbonate solution and the layers separated. The aqueous portion was extracted two additional times with ethyl acetate, then the organics were combined, washed with brine, dried over sodium sulfate, filtered and evaporated to give an oil, 3-(2-dispiro[2.0.2.1]heptan-7-ylethoxy)-1H-pyrazole (3.56 g, 100%). ESI-MS m/z calc. 204.12627, found 205.1 (M+1)⁺; Retention time: 0.59 min (LC method J).

### Step 7: tert-Butyl 2-chloro-6-[3-(2-dispiro[2.0.2.1]heptan-7-ylethoxy)pyrazol-1-yl]pyridine-3-carboxylate

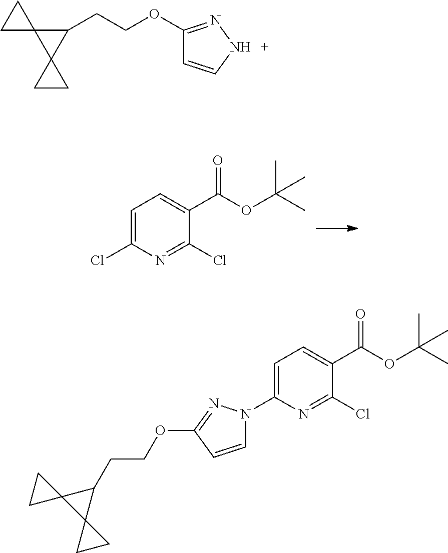 [SMILES: c1cc(OCCC2C3(CC3)C23CC3)n[nH]1 ; CC(C)(C)OC(=O)c1ccc(Cl)nc1Cl ; CC(C)(C)OC(=O)c1ccc(-n2ccc(OCCC3C4(CC4)C34CC4)n2)nc1Cl]

tert-Butyl 2,6-dichloropyridine-3-carboxylate (4.322 g, 17.42 mmol), 3-(2-dispiro[2.0.2.1]heptan-7-ylethoxy)-1H-pyrazole (3.559 g, 17.42 mmol) and potassium carbonate (2.891 g, 20.92 mmol) were combined in anhydrous dimethyl sulfoxide (71.18 mL). 1,4-Diazabicyclo[2.2.2]octane (391.1 mg, 3.487 mmol) was added and the mixture was stirred at room temperature under nitrogen for 16 h. The reaction mixture was diluted with water (136.9 mL) and stirred for 15 min. The resulting white solid was filtered and washed with water. The solid was dissolved in dichloromethane and dried over magnesium sulfate. The mixture was filtered and evaporated to give tert-butyl 2-chloro-6-[3-(2-dispiro[2.0.2.1]heptan-7-ylethoxy)pyrazol-1-yl]pyridine-3-carboxylate (5.69 g, 79%) as a white solid. ¹H NMR (400 MHz, Chloroform-d) δ 8.35 (d, J=2.9 Hz, 1H), 8.18 (d, J=8.4 Hz, 1H), 7.69 (d, J=8.4 Hz, 1H), 5.94 (d, J=2.9 Hz, 1H), 4.25 (s, 2H), 1.90 (d, J=6.8 Hz, 2H), 1.62 (s, 9H), 1.49 (t, J=6.6 Hz, 1H), 0.85 (d, J=1.5 Hz, 4H), 0.65 (d, J=1.5 Hz, 2H), 0.52 (d, J=1.1 Hz, 2H). ESI-MS m/z calc. 415.16626, found 360.0 (M-tBu)⁺; Retention time: 2.09 min (LC method A).

### Step 8: 2-Chloro-6-[3-(2-dispiro[2.0.2.1]heptan-7-ylethoxy)pyrazol-1-yl]pyridine-3-carboxylic acid

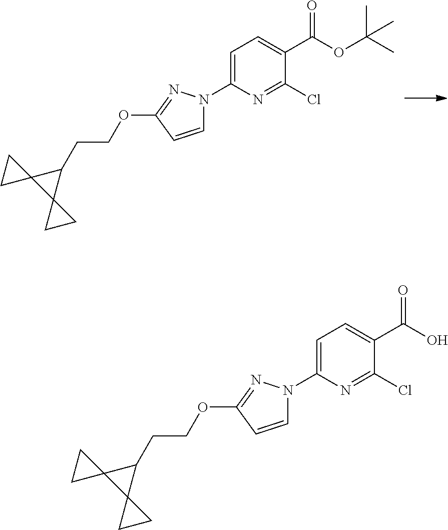 [SMILES: CC(C)(C)OC(=O)c1ccc(-n2ccc(OCCC3C4(CC4)C34CC4)n2)nc1Cl ; O=C(O)c1ccc(-n2ccc(OCCC3C4(CC4)C34CC4)n2)nc1Cl]

tert-Butyl 2-chloro-6-[3-(2-dispiro[2.0.2.1]heptan-7-ylethoxy)pyrazol-1-yl]pyridine-3-carboxylate (5.85 g, 14.07 mmol) was dissolved in dichloromethane (58.5 mL) with trifluoroacetic acid (16.26 mL, 211.1 mmol) and the reaction was stirred at room temperature for 16 h. The reaction was evaporated and to the resulting solid was added diethyl ether and then removed the diethyl ether under reduced pressure. This evaporation from diethyl ether was repeated twice more resulting in a white solid, 2-chloro-6-[3-(2-dispiro[2.0.2.1]heptan-7-ylethoxy)pyrazol-1-yl]pyridine-3-carboxylic acid (5.06 g, 100%). ¹H NMR (400 MHz, Chloroform-d) δ 8.41 (d, J=8.5 Hz, 1H), 8.37 (d, J=2.9 Hz, 1H), 7.75 (d, J=8.5 Hz, 1H), 5.97 (d, J=2.9 Hz, 1H), 4.27 (s, 2H), 1.91 (d, J=6.7 Hz, 2H), 1.50 (s, 1H), 0.85 (d, J=1.5 Hz, 4H), 0.71-0.62 (m, 2H), 0.52 (d, J=1.1 Hz, 2H). ESI-MS m/z calc. 359.10367, found 360.2 (M+1)⁺; Retention time: 2.16 min (LC method A).

### Example 95: Preparation of 2-Chloro-6-(3-fluoro-5-isobutoxy-phenyl)pyridine-3-carboxylic acid

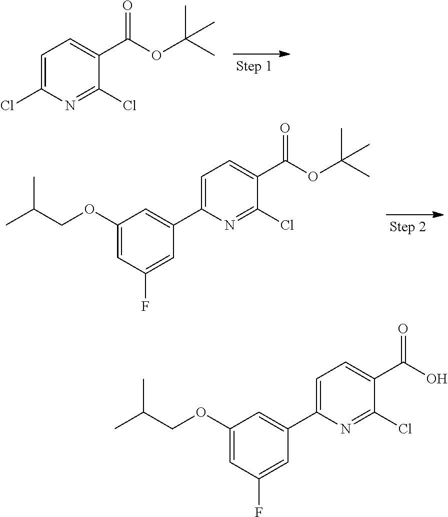

### Step 1: tert-Butyl 2-chloro-6-(3-fluoro-5-isobutoxy-phenyl)pyridine-3-carboxylate

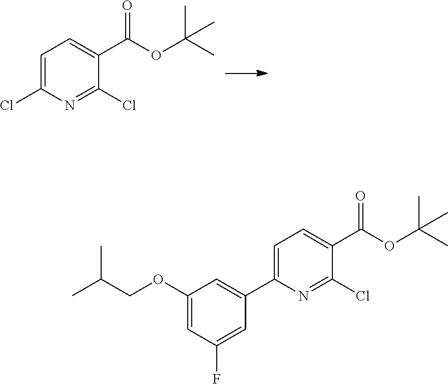 [SMILES: CC(C)(C)OC(=O)c1ccc(Cl)nc1Cl ; CC(C)COc1cc(F)cc(-c2ccc(C(=O)OC(C)(C)C)c(Cl)n2)c1]

tert-Butyl 2,6-dichloropyridine-3-carboxylate (15.0 g, 60.5 mmol) and (3-fluoro-5-isobutoxy-phenyl)boronic acid (13.46 g, 63.48 mmol) were combined and fully dissolved in ethanol (150 mL) and toluene (150 mL). A suspension of sodium carbonate (19.23 g, 181.4 mmol) in water (30 mL) was added. Tetrakis(triphenylphosphine)palladium(0) (2.096 g, 1.814 mmol) was added under nitrogen. The reaction mixture was allowed to stir at 60° C. for 16 h. Volatiles were removed under reduced pressure. The remaining solids were partitioned between water (100 mL) and ethyl acetate (100 mL). The organic layer was washed with brine (1×100 mL), dried over sodium sulfate, filtered and concentrated under reduced pressure. The material was subjected to silica gel column chromatography (0% to 20% ethyl acetate in hexanes gradient). The material was repurified by silica gel chromatography (isocratic 100% hexane for 10 min, then a 0 to 5% ethyl acetate in hexanes gradient) to yield tert-butyl 2-chloro-6-(3-fluoro-5-isobutoxy-phenyl)pyridine-3-carboxylate (18.87 g, 49.68 mmol, 82%), obtained as a colorless oil. ¹H NMR (400 MHz, dimethyl sulfoxide-d₆) 8.24 (d, J=8.0 Hz, 1H), 8.16 (d, J=8.1 Hz, 1H), 7.48 (dd, J=9.4, 2.0 Hz, 2H), 6.99 (dt, J=10.8, 2.2 Hz, 1H), 3.86 (d, J=6.5 Hz, 2H), 2.05 (dt, J=13.3, 6.6 Hz, 1H), 1.57 (d, J=9.3 Hz, 9H), 1.00 (t, J=5.5 Hz, 6H). ESI-MS m/z calc. 379.13504, found 380.2 (M+1)⁺; Retention time: 2.57 min (LC method A).

### Step 2: 2-Chloro-6-(3-fluoro-5-isobutoxy-phenyl)pyridine-3-carboxylic acid

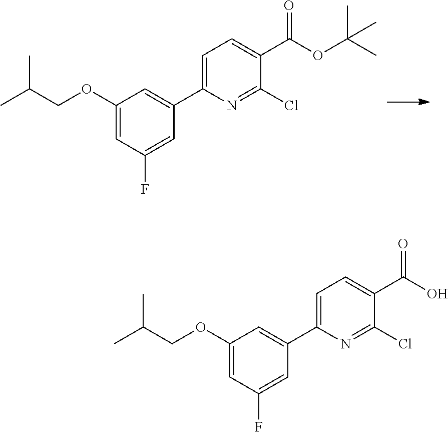

tert-Butyl 2-chloro-6-(3-fluoro-5-isobutoxy-phenyl)pyridine-3-carboxylate (18.57 g, 48.89 mmol) was dissolved in dichloromethane (200 mL). Trifluoroacetic acid (60 mL, 780 mmol) was added and the reaction mixture was allowed to stir at room temperature for 1 h. The reaction mixture was stirred at 40° C. for 2 h. The reaction mixture was concentrated under reduced pressure and taken up in ethyl acetate (100 mL). This solution was washed with a saturated aqueous sodium bicarbonate solution (1×100 mL) and brine (1×100 mL), dried over sodium sulfate, filtered and concentrated under reduced pressure. The crude product was suspended in ethyl acetate (75 mL) and washed with aqueous hydrochloric acid (1 N, 1×75 mL). The organic layer was dried over sodium sulfate, filtered and concentrated under reduced pressure. The resulting solid (17.7 g) was stirred as a slurry in dichloromethane (35 mL) at 40° C. for 30 min. After cooling to room temperature, the slurry was filtered and then rinsed with cold dichloromethane to give 2-chloro-6-(3-fluoro-5-isobutoxy-phenyl)pyridine-3-carboxylic acid (11.35 g, 35.06 mmol, 72%) as a white solid. ¹H NMR (400 MHz, dimethyl sulfoxide-d₆) δ 13.76 (s, 1H), 8.31 (d, J=8.0 Hz, 1H), 8.17 (d, J=8.1 Hz, 1H), 7.54-7.47 (m, 2H), 7.00 (dt, J=10.8, 2.3 Hz, 1H), 3.87 (d, J=6.5 Hz, 2H), 2.05 (dt, J=13.3, 6.6 Hz, 1H), 1.01 (d, J=6.7 Hz, 6H). ESI-MS m/z calc. 323.1, found 324.1 (M+1)⁺; Retention time: 1.96 min (LC method A).

### Example 96: Preparation of (14S)-12,12-dimethyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5,7,9,19,21-hexaene-2,2,4-trione

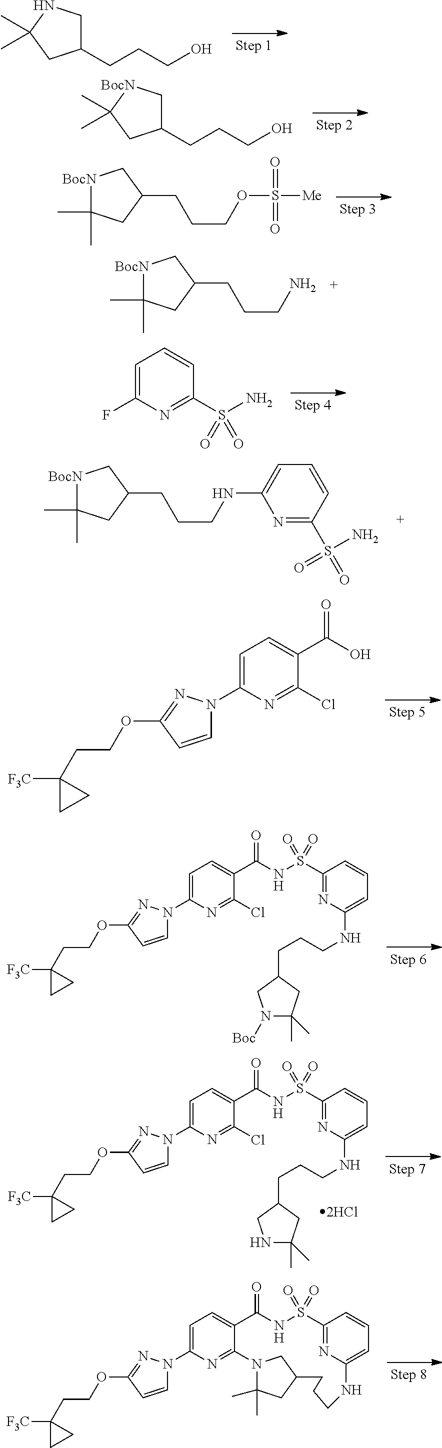

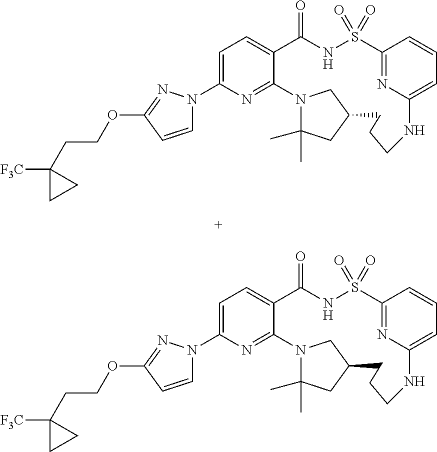 [SMILES: CC1(C)C[C@H]2CCCNc3cccc(n3)S(=O)(=O)NC(=O)c3ccc(-n4ccc(OCCC5(C(F)(F)F)CC5)n4)nc3N1C2 ; CC1(C)C[C@@H]2CCCNc3cccc(n3)S(=O)(=O)NC(=O)c3ccc(-n4ccc(OCCC5(C(F)(F)F)CC5)n4)nc3N1C2]

### Step 1: tert-Butyl 4-(3-hydroxypropyl)-2,2-dimethyl-pyrrolidine-1-carboxylate

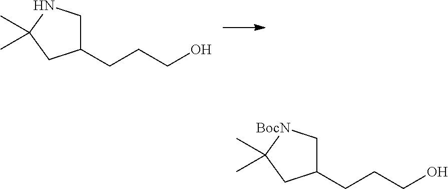 [SMILES: CC1(C)CC(CCCO)CN1 ; CC(C)(C)OC(=O)N1CC(CCCO)CC1(C)C]

A 1 L, 3-neck round bottom flask was fitted with a mechanical stirrer, a cooling bath, an addition funnel, a J-Kem temperature probe and a nitrogen inlet/outlet. The vessel was charged under a nitrogen atmosphere with 3-(5,5-dimethylpyrrolidin-3-yl)propan-1-ol (15 g, 95.39 mmol) and dichloromethane (225 mL, 15 mL/g) which provided a clear light yellow solution. Stirring was commenced and the pot temperature was recorded at 19° C. The cooling bath was charged with crushed ice/water and the pot temperature was lowered to 0° C. The addition funnel was charged with triethylamine (12.55 g, 124.0 mmol) which was subsequently added neat dropwise over 5 min. No exotherm was observed. The addition funnel was then charged with di-tert-butyl dicarbonate (22.89 g, 104.9 mmol) dissolved in dichloromethane (225 mL). The clear pale yellow solution was then added dropwise over 30 min which resulted in gentle gas evolution. No exotherm was observed. The cooling bath was removed and the resulting clear light yellow solution was allowed to warm to room temperature and continue to stir at room temperature for 3 h. The reaction mixture was transferred to a separatory funnel and partitioned with water (75 mL). The organic was removed and washed with saturated sodium chloride solution (75 mL), dried over sodium sulfate (150 g) and then filtered through a glass frit Buchner funnel. The filtrate was concentrated under reduced pressure to provide (30 g) of a clear light yellow oil as the desired crude product. The material was purified by silica gel column flash chromatography (liquid load with dichloromethane) eluting with a gradient of 100% dichloromethane to 10% methyl alcohol in dichloromethane over 60 min collecting 50 mL fractions. The desired product fractions were combined and concentrated under reduced pressure to provide tert-butyl 4-(3-hydroxypropyl)-2,2-dimethyl-pyrrolidine-1-carboxylate (22 g, 0.0855 mol, 90% yield) as a clear pale yellow viscous oil. ¹H NMR (400 MHz, DMSO-d₆) δ 4.38 (td, J=5.2, 1.4 Hz, 1H), 3.54 (dt, J=10.3, 6.7 Hz, 1H), 3.38 (td, J 6.6, 3.5 Hz, 2H), 2.76 (q, J=10.3 Hz, 1H), 2.07 (td, J=11.6, 5.7 Hz, 1H), 1.87 (ddd, J 16.7, 12.1, 6.0 Hz, 1H), 1.37 (dd, J=14.2, 10.4 Hz, 17H), 1.24 (s, 3H).

### Step 2: tert-Butyl 2,2-dimethyl-4-(3-methylsulfonyl oxypropyl)pyrrolidine-1-carboxylate

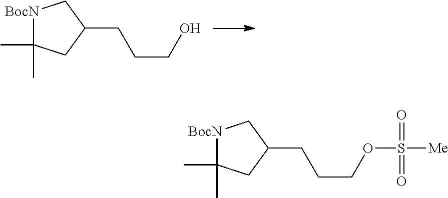 [SMILES: CC(C)(C)OC(=O)N1CC(CCCO)CC1(C)C ; CC(C)(C)OC(=O)N1CC(CCCOS(C)(=O)=O)CC1(C)C]

tert-Butyl 4-(3-hydroxypropyl)-2,2-dimethyl-pyrrolidine-1-carboxylate (50.5 g, 196.22 mmol) and triethylamine (39.711 g, 54.698 mL, 392.44 mmol) were dissolved in dichloromethane (500 mL) and the resulting solution was chilled in an ice water bath for 30 min. Mesyl chloride (24.725 g, 16.706 mL, 215.84 mmol) was added dropwise over a 30 min period, then the ice bath was removed and the mixture stirred at room temperature for one hour. The reaction was then quenched with saturated sodium bicarbonate solution (200 mL). The phases were separated and the organic phase was extracted with saturated sodium bicarbonate (200 mL) and water (2×100 mL). The aqueous phases were discarded and the organic phase was dried over sodium sulfate, filtered and concentrated in vacuo to obtain tert-butyl 2,2-dimethyl-4-(3-methylsulfonyl oxypropyl)pyrrolidine-1-carboxylate (64.2 g, 93%) as a pale yellow oil. ESI-MS m/z calc. 335.1766, found 336.4 (M+1)⁺; Retention time: 5.54 min (LC method C).

### Step 3: tert-Butyl 4-(3-aminopropyl)-2,2-dimethyl-pyrrolidine-1-carboxylate

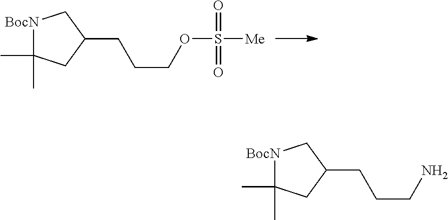 [SMILES: CC(C)(C)OC(=O)N1CC(CCCOS(C)(=O)=O)CC1(C)C ; CC(C)(C)OC(=O)N1CC(CCCN)CC1(C)C]

tert-Butyl 2,2-dimethyl-4-(3-methylsulfonyloxypropyl)pyrrolidine-1-carboxylate (64.2 g, 191.38 mmol) was dissolved in dioxane (650 mL) and then ammonium hydroxide (650 mL) was added and the resulting mixture heated to 45° C. for 18 h. After 18 h, the reaction was cooled to room temperature. The solution was diluted with 1M sodium hydroxide (200 mL) and then extracted with diethyl ether (3×650 mL). The aqueous phase was discarded and the combined organic phases were extracted with water (2×200 mL). The aqueous phases were discarded and the organic phase was dried over sodium sulfate, filtered and concentrated in vacuo to afford tert-butyl 4-(3-aminopropyl)-2,2-dimethyl-pyrrolidine-1-carboxylate (48.9 g, 95%) as a pale yellow oil. ESI-MS m/z calc. 256.2151, found 257.3 (M+1)⁺; Retention time: 3.70 min (LC method C).

### Step 4: tert-Butyl 2,2-dimethyl-4-[3-[(6-sulfamoyl-2-pyridyl)amino]propyl]pyrrolidine-1-carboxylate

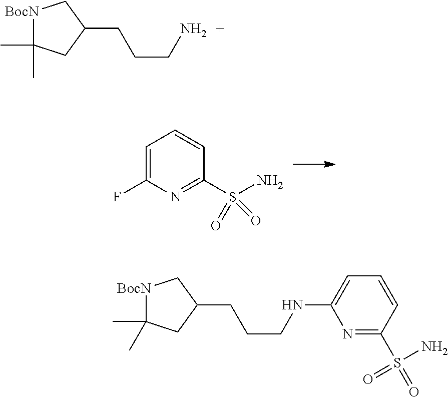 [SMILES: CC(C)(C)OC(=O)N1CC(CCCN)CC1(C)C ; NS(=O)(=O)c1cccc(F)n1 ; CC(C)(C)OC(=O)N1CC(CCCNc2cccc(S(N)(=O)=O)n2)CC1(C)C]

To tert-butyl 4-(3-aminopropyl)-2,2-dimethyl-pyrrolidine-1-carboxylate (8.91 g, 34.8 mmol) and 6-fluoropyridine-2-sulfonamide (6.13 g, 34.8 mmol) in dimethyl sulfoxide (75 mL) was added potassium carbonate (4.91 g, 35.5 mmol) and the mixture stirred at 100° C. for 12 h and then allowed to cool to ambient temperature and stirred for an additional 4 h (16 h total). The reaction mixture was slowly poured into hydrochloric acid (35 mL of 1 M, 35.00 mmol) in water (200 mL) (some foaming) and diluted with ethyl acetate (250 mL). The organic phase was separated and washed with 100 mL of brine. The organic phase was dried over magnesium sulfate, filtered over celite, and concentrated in vacuo to afford a dark yellow oil. The crude product was purified by silica gel chromatography eluting with 0%-100% ethyl acetate in hexanes. Collected both pure (9.0 g) and impure (3 g) fractions. Purified the impure fractions by silica gel chromatography eluting with 0%-100% ethyl acetate in hexanes affording, in total, tert-butyl 2,2-dimethyl-4-[4-[(6-sulfamoyl-2-pyridyl)amino]propyl]pyrrolidine-1-carboxylate (10.0 g, 69%). ¹H NMR (400 MHz, dimethyl sulfoxide-d₆) δ 7.52 (dd, J=8.5, 7.2 Hz, 1H), 7.07 (s, 2H), 6.95 (dd, J=7.2, 0.7 Hz, 2H), 6.61 (d, J=8.5 Hz, 1H), 3.55 (q, J=9.1 Hz, 1H), 3.32-3.24 (m, 2H), 2.79 (q, J=10.0 Hz, 1H), 2.13 (d, J=16.1 Hz, 1H), 1.96-1.82 (m, 1H), 1.51 (dt, J=18.0, 9.3 Hz, 2H), 1.37 (dd, J=12.9, 10.6 Hz, 15H), 1.24 (s, 3H). ESI-MS m/z calc. 412.21442, found 413.1 (M+1)⁺; Retention time: 2.34 min (LC method K).

### Step 5: tert-Butyl 4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]propyl]-2,2-dimethyl-pyrrolidine-1-carboxylate

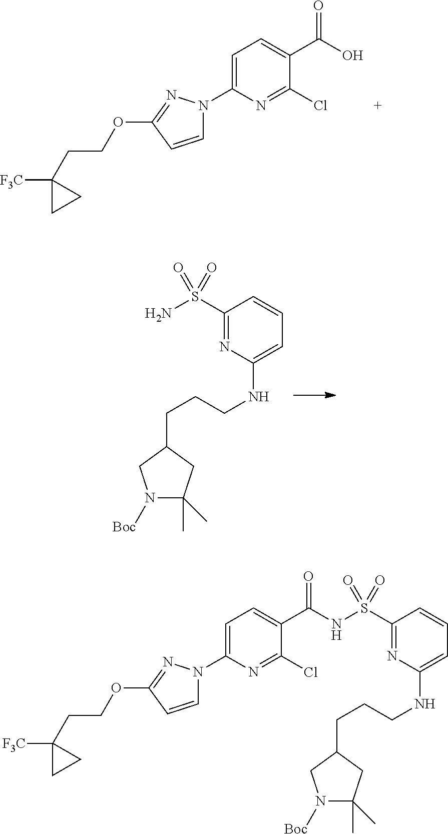 [SMILES: O=C(O)c1ccc(-n2ccc(OCCC3(C(F)(F)F)CC3)n2)nc1Cl ; CC(C)(C)OC(=O)N1CC(CCCNc2cccc(S(N)(=O)=O)n2)CC1(C)C ; CC(C)(C)OC(=O)N1CC(CCCNc2cccc(S(=O)(=O)NC(=O)c3ccc(-n4ccc(OCCC5(C(F)(F)F)CC5)n4)nc3Cl)n2)CC1(C)C]

Part A: To 2-chloro-6-[3-[2-[1-(trifluoromethyl)cyclopropyl]ethoxy]pyrazol-1-yl]pyridine-3-carboxylic acid (11.4 g, 30.34 mmol) in tetrahydrofuran (150 mL) was slowly added carbonyl diimidazole (5.9 g, 36 mmol). The mixture was stirred at ambient temperature for 1 hr. Additional carbonyl diimidazole (0.5 g, 3 mmol) was added and the reaction was stirred at ambient temperature for an additional 1 h (2 h total).

Part B: To the activated ester prepared in Part A was added tert-butyl 2,2-dimethyl-4-[3-[(6-sulfamoyl-2-pyridyl)amino]propyl]pyrrolidine-1-carboxylate (10.0 g, 24.24 mmol) in tetrahydrofuran (50 mL) followed by 1,8-diazabicyclo[5.4.0]undec-7-ene (7.5 mL, 50. mmol) and the mixture stirred at ambient temperature for 16 hr. Citric acid (25.1 g, 130.6 mmol) in water (200 mL) was slowly added to the reaction mixture and acidified to ˜pH=3. The mixture became cloudy but no precipitate formed. The mixture was extracted with ethyl acetate (400 mL) and the organic phase washed with brine, dried over magnesium sulfate, filtered over celite, and concentrated in vacuo. The crude product was purified by silica gel chromatography eluting with 0-80% ethyl acetate/hexanes affording the product as a foam, tert-butyl 4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]propyl]-2,2-dimethyl-pyrrolidine-1-carboxylate (13.82 g, 74%)¹H NMR (400 MHz, dimethyl sulfoxide-d₆) δ 12.79 (s, 1H), 8.40 (t, J=2.7 Hz, 1H), 8.11 (d, J 8.4 Hz, 1H), 7.71 (d, J=8.3 Hz, 1H), 7.62 (dd, J=8.5, 7.2 Hz, 1H), 7.21 (d, J=5.8 Hz, 1H), 7.17 (d, J=7.1 Hz, 1H), 6.74 (d, J=8.5 Hz, 1H), 6.19 (t, J=2.3 Hz, 1H), 4.34 (t, J=7.1 Hz, 2H), 3.50 (dt, J=18.3, 9.0 Hz, 1H), 3.23 (d, J=6.7 Hz, 2H), 2.80-2.69 (m, 1H), 2.09 (t, J 7.1 Hz, 2H), 1.79 (td, J 12.4, 6.1 Hz, 1H), 1.52 (dt, J 15.4, 8.0 Hz, 2H), 1.36 (d, J=11.4 Hz, 10H), 1.33-1.25 (m, 5H), 1.17 (s, 2H), 0.99-0.92 (m, 2H), 0.89 (d, J=5.0 Hz, 2H). ESI-MS m/z calc. 769.2636, found 770.1 (M+1)⁺; Retention time: 3.48 min (LC method K).

### Step 6: 2-chloro-{N}-[[6-[3-(5,5-dimethylpyrrolidin-3-yl)propylamino]-2-pyridyl]sulfonyl]-6-[3-[2-[1-(trifluoromethyl)cyclopropyl]ethoxy]pyrazol-1-yl]pyridine-3-carboxamide (dihydrochloride salt)

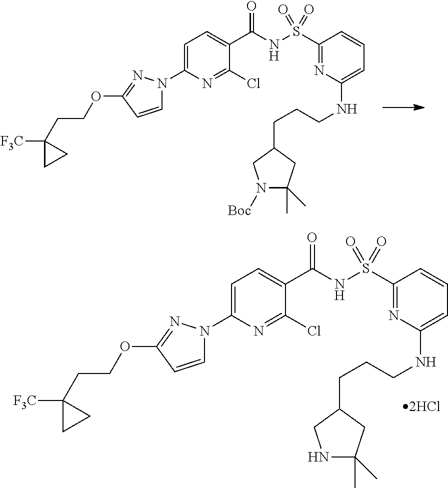

To tert-butyl 4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]propyl]-2,2-dimethyl-pyrrolidine-1-carboxylate (13.8 g, 17.92 mmol) in dichloromethane (75 mL) was added hydrochloric acid (15 mL of 4 M, 60.00 mmol) and the mixture stirred at ambient temperature for 16 hr. The solvent was removed in vacuo and the residue diluted with 100 mL of ethyl acetate. The solvent was removed in vacuo and repeated with an additional 100 mL of ethyl acetate affording a foam, 2-chloro-{N}-[[6-[3-(5,5-dimethylpyrrolidin-3-yl)propylamino]-2-pyridyl]sulfonyl]-6-[3-[2-[1-(trifluoromethyl)cyclopropyl]ethoxy]pyrazol-1-yl]pyridine-3-carboxamide (dihydrochloride salt) (13.5 g, 101%). ¹H NMR (400 MHz, dimethyl sulfoxide-d₆) δ 12.83 (s, 1H), 9.06 (s, 1H), 8.92 (s, 1H), 8.42 (d, J=2.8 Hz, 1H), 8.11 (d, J=8.4 Hz, 1H), 7.72 (d, J=8.4 Hz, 1H), 7.63 (dd, J=8.5, 7.2 Hz, 1H), 7.29 (s, 1H), 7.18 (dd, J=7.2, 0.7 Hz, 1H), 6.76 (dd, J=8.5, 0.7 Hz, 1H), 6.21 (d, J=2.9 Hz, 1H), 4.95 (s, 2H), 4.35 (t, J=7.0 Hz, 2H), 3.41-3.28 (m, 1H), 3.24 (p, J=6.7 Hz, 2H), 2.80 (tt, J=11.8, 6.7 Hz, 1H), 2.35 (q, J=7.8, 7.3 Hz, 1H), 2.09 (t, J=7.1 Hz, 2H), 1.91 (dd, J=12.8, 7.6 Hz, 1H), 1.58-1.43 (m, 3H), 1.42-1.31 (m, 5H), 1.24 (s, 3H), 1.01-0.93 (m, 2H), 0.90 (dd, J=7.9, 3.2 Hz, 2H). ESI-MS m/z calc. 669.2112, found 670.0 (M+1)⁺; Retention time: 2.26 min (LC method K).

### Step 7: 12,12-Dimethyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione

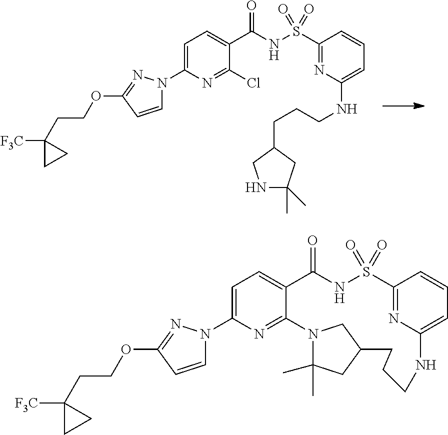

To the 2-chloro-N-[[6-[3-(5,5-dimethylpyrrolidin-3-yl)propylamino]-2-pyridyl]sulfonyl]-6-[3-[2-[1-(trifluoromethyl)cyclopropyl]ethoxy]pyrazol-1-yl]pyridine-3-carboxamide (dihydrochloride salt) (12.9 g, 17.36 mmol) in NMP (120 mL) was added potassium carbonate (9.64 g, 69.8 mmol) followed by cesium fluoride (2.69 g, 17.7 mmol) and the slurry was stirred at 150° C. for 16 hr. The reaction was then allowed to cool to ambient temperature. The mixture was diluted with water (100 mL) and poured into water (400 mL) and the mixture was slowly acidified with hydrochloric acid (27.0 mL of 6 M, 162.0 mmol). The precipitate was collected using a medium frit and washed 3× with 50 mL of water. The solid was air dried for 1 h and then dissolved in ethyl acetate (400 mL). The organic phase was concentrated in vacuo and the crude product purified by silica gel chromatography eluting with 0-100% ethyl acetate/hexanes. 6.3 g of pure product and 6 g of impure fractions were collected. The impure fractions were purified by silica gel chromatography eluting with 0-5% methanol/dichloromethane which provided good separation of product from impurities. The pure product fractions were combined affording 12,12-dimethyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (7.4 g, 67%). ¹H NMR (400 MHz, dimethyl sulfoxide-d₆) δ 12.52 (s, 1H), 8.22 (d, J=2.8 Hz, 1H), 7.82 (d, J=8.2 Hz, 1H), 7.58 (dd, J=8.5, 7.2 Hz, 1H), 7.05 (d, J=7.0 Hz, 1H), 7.00 (s, 1H), 6.92 (d, J=8.2 Hz, 1H), 6.71 (d, J=8.2 Hz, 1H), 6.11 (d, J=2.8 Hz, 1H), 4.31 (t, J=7.0 Hz, 2H), 4.02-3.81 (m, 1H), 3.15 (dt, J=10.1, 3.8 Hz, 1H), 2.95 (d, J=13.7 Hz, 1H), 2.78-2.61 (m, 1H), 2.15 (dd, J=5.7, 2.9 Hz, 1H), 2.07 (d, J=6.4 Hz, 2H), 1.92-1.82 (m, 1H), 1.81-1.69 (m, 1H), 1.64-1.53 (m, 6H), 1.51 (s, 3H), 1.36-1.23 (m, 1H), 0.99-0.93 (m, 2H), 0.90 (d, J=10.8 Hz, 2H). ESI-MS m/z calc. 633.2345, found 634.2 (M+1)⁺; Retention time: 2.23 min (LC method L).

### Step 8: (14R)-12,12-Dimethyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5,7,9,19,21-hexaene-2,2,4-trione and (14S)-12,12-dimethyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5,7,9,19,21-hexaene-2,2,4-trione

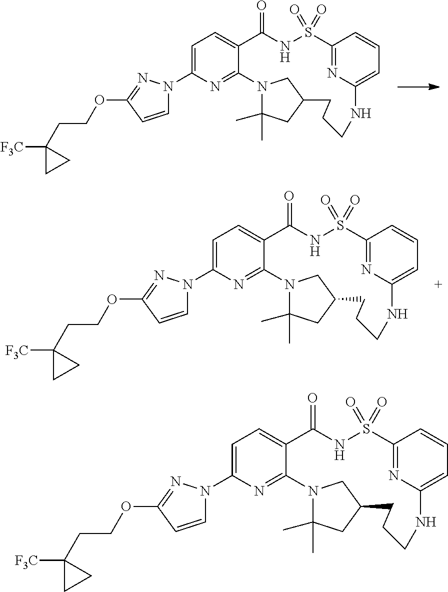 [SMILES: CC1(C)CC2CCCNc3cccc(n3)S(=O)(=O)NC(=O)c3ccc(-n4ccc(OCCC5(C(F)(F)F)CC5)n4)nc3N1C2 ; CC1(C)C[C@H]2CCCNc3cccc(n3)S(=O)(=O)NC(=O)c3ccc(-n4ccc(OCCC5(C(F)(F)F)CC5)n4)nc3N1C2 ; CC1(C)C[C@@H]2CCCNc3cccc(n3)S(=O)(=O)NC(=O)c3ccc(-n4ccc(OCCC5(C(F)(F)F)CC5)n4)nc3N1C2]

Racemic 12,12-dimethyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (7.4 g) was dissolved in 150 mL of acetonitrile and subjected to chiral SFC purification. The sample was separated by chiral SFC chromatography using a ChiralPak AS-H (250×21.2 mm column, 5 μm particle size) with 25% acetonitrile:methanol (90:10))/75% carbon dioxide mobile phase at 70 mL/min giving as the first enantiomer to elute, (14R)-12,12-dimethyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5,7,9,19,21-hexaene-2,2,4-trione (2.91 g, 53%); ¹H NMR (400 MHz, dimethyl sulfoxide-d₆) δ 12.51 (s, 1H), 8.21 (d, J 2.7 Hz, 1H), 7.82 (d, J=8.2 Hz, 1H), 7.58 (t, J=7.8 Hz, 1H), 7.05 (d, J=7.0 Hz, 1H), 6.99 (d, J=5.8 Hz, 1H), 6.92 (d, J=8.2 Hz, 1H), 6.71 (d, J=8.5 Hz, 1H), 6.11 (d, J 2.7 Hz, 1H), 4.31 (t, J=7.0 Hz, 2H), 3.93 (s, 1H), 3.16 (s, 1H), 2.95 (d, J=12.7 Hz, 1H), 2.80-2.64 (m, 1H), 2.10 (s, 1H), 2.08 (t, J=7.1 Hz, 2H), 1.86 (dd, J=11.5, 5.4 Hz, 1H), 1.81-1.71 (m, 1H), 1.60 (s, 6H), 1.51 (s, 3H), 1.31 (d, J=12.8 Hz, 1H), 0.95 (d, J=4.3 Hz, 2H), 0.89 (s, 2H); ESI-MS m/z calc. 633.2345, found 634.3 (M+1)⁺; Retention time: 2.19 min; and as the second enantiomer to elute, (14S)-12,12-dimethyl-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(23),5,7,9,19,21-hexaene-2,2,4-trione (3.0 g, 54%) ¹H NMR (400 MHz, dimethyl sulfoxide-d₆) δ 12.52 (s, 1H), 8.22 (d, J=2.8 Hz, 1H), 7.82 (d, J 8.2 Hz, 1H), 7.58 (dd, J 8.5, 7.1 Hz, 1H), 7.05 (d, J 7.2 Hz, 1H), 6.99 (s, 1H), 6.92 (d, J=8.2 Hz, 1H), 6.71 (d, J=8.5 Hz, 1H), 6.11 (d, J=2.8 Hz, 1H), 4.31 (t, J=7.0 Hz, 2H), 3.92 (d, J=12.6 Hz, 1H), 3.15 (s, 1H), 2.95 (d, J=13.2 Hz, 1H), 2.78-2.64 (m, 1H), 2.08 (t, J=7.1 Hz, 3H), 1.86 (dd, J=11.9, 5.3 Hz, 1H), 1.77 (s, 1H), 1.60 (s, 6H), 1.51 (s, 3H), 1.31 (q, J=11.9 Hz, 1H), 1.00-0.92 (m, 2H), 0.90 (d, J=10.4 Hz, 2H). ESI-MS m/z calc. 633.2345, found 634.1 (M+1)⁺; Retention time: 2.2 min (LC method A).

### Example 97: Preparation of tert-butyl (4S)-4-[3-(4-tert-butyl-2-pyridyl)-3-[(6-sulfamoyl-2-pyridyl)amino]propyl]-2,2-dimethyl-pyrrolidine-1-carboxylate

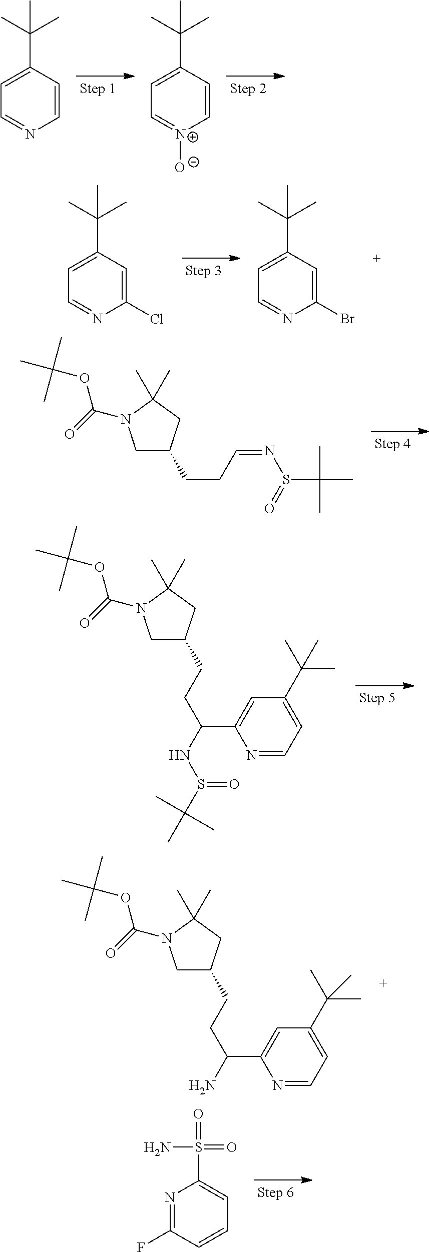

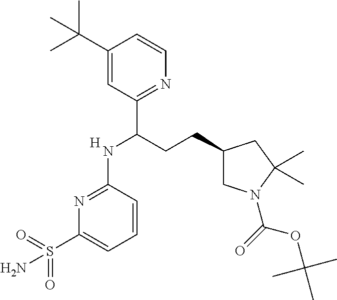

### Step 1: 4-tert-Butyl-1-oxido-pyridin-1-ium

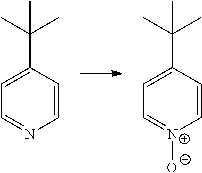

In a 2 L round bottom flask, 4-tert-butylpyridine (73.030 g, 81 mL, 529.34 mmol) was added to glacial acetic acid (600 mL). Next, hydrogen peroxide (30% in water, 450 mL) was added and the reaction mixture was refluxed under air for 4 hr. Additional hydrogen peroxide (30% in water, 450 mL) was added and the reflux was continued overnight (16 h). The solvent was removed in a rotary evaporator, and the remaining solution was neutralized with a saturated sodium carbonate solution and then extracted with methylene chloride (2×500 mL) plus chloroform:isopropanol (3:1, v:v, 3×200 mL). The combined organics were dried over MgSO₄, filtered, and concentrated to give crude 4-tert-butyl-1-oxido-pyridin-1-ium (89 g, 100%). ESI-MS m/z calc. 151.0997, found 152.4 (M+1)⁺; Retention time: 1.92 minutes (LC method B).

### Step 2: 4-tert-Butyl-2-chloro-pyridine

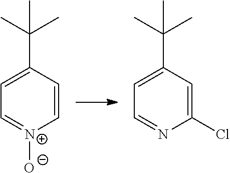

4-tert-Butyl-1-oxido-pyridin-1-ium (50.6 g, 301.18 mmol) was placed in the reaction flask and was cooled in an ice bath. POCl₃ (250 mL) was slowly added to the reaction flask to obtain a mixture. The mixture was heated under reflux for 20 hours. Thereafter, the temperature was slowly reduced to 20° C., the solvent was removed from the mixture by evaporation under vacuum, then a sodium carbonate aqueous solution was added for neutralization, and the contents were extracted using ethyl acetate (2×800 mL). The organic layer was washed by brine, dried over sodium sulfate and concentrated. The crude residue was purified by silica gel flash chromatography using 0 to 30% ethyl acetate in hexane to afford 4-tert-butyl-2-chloro-pyridine (38.6 g, 72%) as an amber liquid. ¹H NMR (500 MHz, DMSO-d₆) δ 8.32 (dd, J=5.3, 0.7 Hz, 1H), 7.48-7.40 (m, 2H), 1.27 (s, 9H). ESI-MS m/z calc. 169.0658, found 170.3 (M+1)⁺; Retention time: 3.11 minutes (LC method B).

### Step 3: 2-Bromo-4-tert-butyl-pyridine

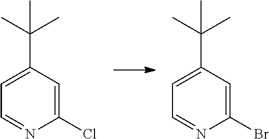

A solution of 4-tert-butyl-2-chloro-pyridine (27.07 g, 151.58 mmol) and trimethylsilyl bromide (170.52 g, 150 mL, 1.092 mol) in propionitrile (450 mL) was stirred under reflux for 21 hr. The reaction flask was vacuum pumped to remove the solvents, a sodium carbonate aqueous solution was added for neutralization, and the contents in the reaction flask were extracted using ethyl acetate (2×800 mL). The organic layer was washed by brine, dried over sodium sulfate and concentrated. The crude residue was purified by silica gel flash chromatography using 0 to 30% ethyl acetate in hexane to afford 2-bromo-4-tert-butyl-pyridine (34.2 g, 100%) as an amber liquid. ¹H NMR (500 MHz, DMSO-d₆) δ 8.29 (d, J=5.3 Hz, 1H), 7.58 (s, 1H), 7.46 (dd, J=5.2, 1.7 Hz, 1H), 1.27 (s, 9H). ESI-MS m/z calc. 213.0153, found 214.3 (M+1)⁺; Retention time: 5.01 minutes (LC method C).

### Step 4: tert-Butyl (4S)-4-[3-(4-tert-butyl-2-pyridyl)-3-(tert-butylsulfinylamino)propyl]-2,2-dimethyl-pyrrolidine-1-carboxylate

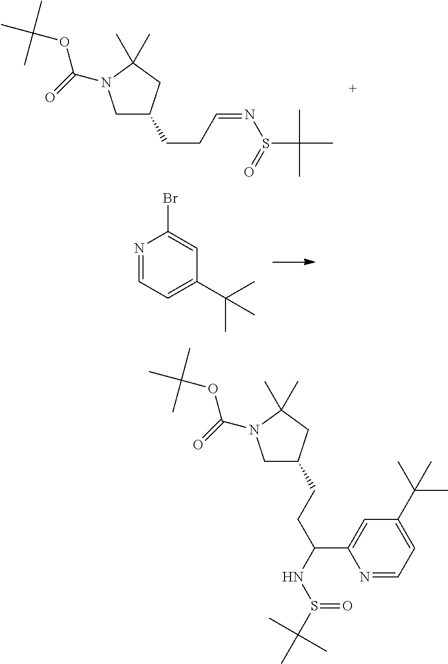

2-Bromo-4-tert-butyl-pyridine (6 g, 26.623 mmol) was dissolved in diethyl ether (60 mL) and the solution was cooled in a dry ice acetone bath (<−70° C.) under a nitrogen atmosphere. n-BuLi (12 mL of 2.5 M in hexanes, 30.00 mmol) was added dropwise. The mixture was stirred in the cooling bath for 40 min. tert-Butyl (4S)-4[(3Z)-3-tert-butylsulfinyliminopropyl]-2,2-dimethyl-pyrrolidine-1-carboxylate (4.45 g, 11.79 mmol) was added as a THF (5 mL) solution. The mixture was stirred at −78° C. to −40° C. for 45 min. Saturated aqueous NH₄Cl (80 mL) was added. The mixture was allowed to warm to rt and partitioned between water (80 mL) and EtOAc (100 mL). This quenched mixture was separated in two layers and the aqueous layer was extracted once with EtOAc (100 mL). The combined organics were washed with brine, dried over anhydrous Na₂SO₄, filtered and concentrated. The residue was purified by silica gel flash chromatography using 0 to 20% ethyl acetate in hexane to afford tert-butyl (4S)-4-[3-(4-tert-butyl-2-pyridyl)-3-(tert-butylsulfinylamino)propyl]-2,2-dimethyl-pyrrolidine-1-carboxylate (4.87 g, 79%). ESI-MS m/z calc. 493.3338, found 494.6 (M+1)⁺; Retention time: 3.01 minutes (LC method B).

### Step 5: tert-Butyl (4S)-4-[3-amino-3-(4-tert-butyl-2-pyridyl)propyl]-2,2-dimethyl-pyrrolidine-1-carboxylate

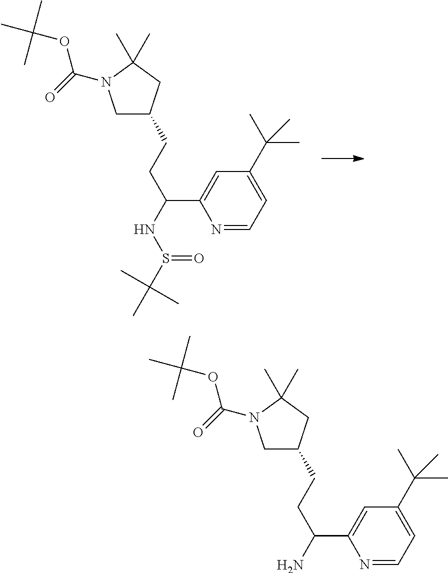

tert-Butyl (4S)-4-[3-(4-tert-butyl-2-pyridyl)-3-(tert-butylsulfinylamino)propyl]-2,2-dimethyl-pyrrolidine-1-carboxylate (4.87 g, 9.37 mmol) was dissolved in THF (43 mL) and water (8.3 mL). Molecular iodine (750 mg, 2.95 mmol) was added. The mixture was stirred at 52° C. for 3 hours. It was then cooled to rt and partitioned between EtOAc (200 mL) and Na₂S₂O₃ (60 g) in saturated aqueous NaHCO₃(200 mL). The layers were separated and the aqueous layer was extracted one more time with EtOAc (100 mL). The organic layer was washed with brine, dried over anhydrous Na₂SO₄, filtered and concentrated to give tert-butyl (4S)-4-[3-amino-3-(4-tert-butyl-2-pyridyl)propyl]-2,2-dimethyl-pyrrolidine-1-carboxylate (3.9 g, 102%). ESI-MS m/z calc. 389.3042, found 390.7 (M+1)⁺; Retention time: 2.94 minutes (LC method B).

### Step 6: tert-Butyl (4S)-4-[3-(4-tert-butyl-2-pyridyl)-3-[(6-sulfamoyl-2-pyridyl)amino]propyl]-2,2-dimethyl-pyrrolidine-1-carboxylate

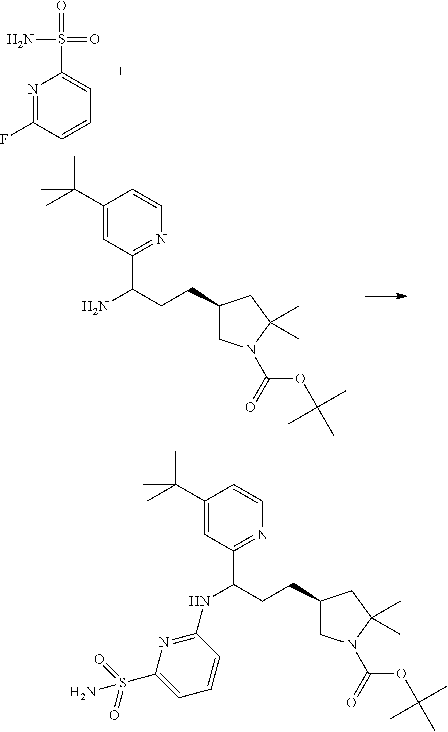

To a mixture of tert-butyl (4S)-4-[3-amino-3-(4-tert-butyl-2-pyridyl)propyl]-2,2-dimethyl-pyrrolidine-1-carboxylate (3.9 g, 9.5104 mmol) and 6-fluoropyridine-2-sulfonamide (3 g, 16.178 mmol) in DMSO (10 mL) was added DIEA (5 mL, 28.706 mmol). The mixture was stirred at 115° C. for 24 hours. The reaction mixture was cooled to rt. and then diluted with water (100 mL) and EtOAc (100 mL). The layers were separated and the organic layer was washed with brine (2×100 mL), dried over anhydrous Na₂SO₄, and concentrated. The residue was purified by silica gel flash chromatography (120 g column), using 0-80% EtOAc in hexanes to afford tert-butyl (4S)-4-[3-(4-tert-butyl-2-pyridyl)-3-[(6-sulfamoyl-2-pyridyl)amino]propyl]-2,2-dimethyl-pyrrolidine-1-carboxylate (3.66 g, 67%) as a pale color solid. ¹H NMR (500 MHz, DMSO-d₆) δ 8.43 (d, J=5.2 Hz, 1H), 7.55-7.48 (m, 2H), 7.44 (d, J=9.5 Hz, 1H), 7.24 (dd, J=5.2, 1.9 Hz, 1H), 7.07 (s, 2H), 6.95 (d, J=7.2 Hz, 1H), 6.73 (d, J=8.5 Hz, 1H), 5.22 (s, 1H), 3.51 (p, J=8.9 Hz, 1H), 2.73 (dtd, J=21.5, 10.6, 4.4 Hz, 1H), 2.05 (s, 1H), 1.94-1.73 (m, 3H), 1.42-1.35 (m, 10H), 1.32 (dd, J=12.4, 3.0 Hz, 4H), 1.26 (d, J=0.9 Hz, 9H), 1.21 (s, 3H). ESI-MS m/z calc. 545.3036, found 546.1 (M+1)⁺; Retention time: 2.19 minutes (LC method H).

### Example 98: Preparation of N-[3-[(3S)-5,5-dimethyl-1-(2,2,2-trifluoroacetyl)pyrrolidin-3-yl]propylidene]-2-methyl-propane-2-sulfinamide

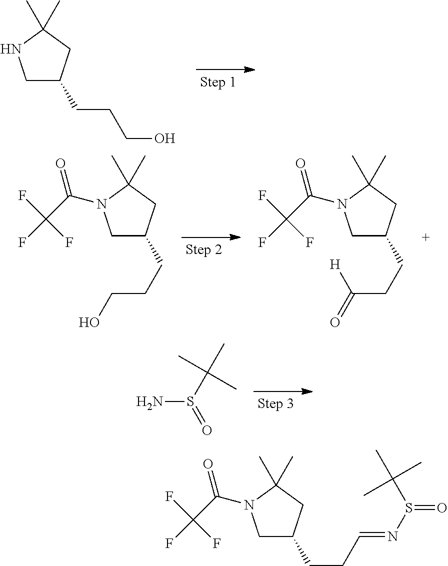

### Step 1: 2,2,2-Trifluoro-1-[(4S)-4-(3-hydroxypropyl)-2,2-dimethyl-pyrrolidin-1-yl]ethenone

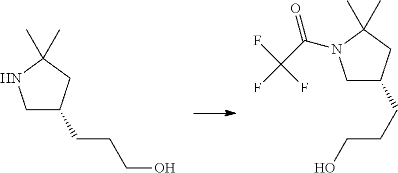 [SMILES: CC1(C)C[C@H](CCCO)CN1 ; CC1(C)C[C@H](CCCO)CN1C(=O)C(F)(F)F]

3-[(3S)-5,5-Dimethylpyrrolidin-3-yl]propan-1-ol (8.3 g, 50.142 mmol) was dissolved in DCM (100 mL). NEt₃ (14 mL, 100.44 mmol) was added and the mixture was stirred under nitrogen balloon in ice water bath for 5 min. Trifluoroacetic anhydride (11 mL, 78.036 mmol) was added via syringe dropwise over 5 min. The mixture was allowed to stir at rt for 2h. It was then concentrated and the residue was taken into a mixture of MeOH and THF (20 mL each). A LiOH (1 g, 41.757 mmol) solution in water (20 mL) was added. The mixture was stirred at rt for 2h. The mixture was concentrated and then partitioned between water and DCM (50 mL each). The layers were separated and the aqueous layer was further extracted with DCM (50 mL×2). The combined organics were dried over anhydrous MgSO₄, filtered and concentrated in vacuo to afford 2,2,2-trifluoro-1-[(4S)-4-(3-hydroxypropyl)-2,2-dimethyl-pyrrolidin-1-yl]ethanone (10.3 g, 77%). ESI-MS m/z calc. 253.129, found 254.3 (M+1)⁺; Retention time: 2.41 minutes (LC method B).

### Step 2: 3-[(3S)-5,5-Dimethyl-1-(2,2,2-trifluoroacetyl)pyrrolidin-3-yl]propanal

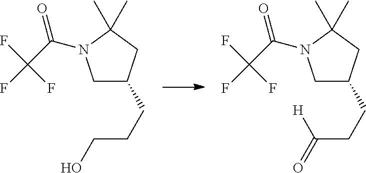 [SMILES: CC1(C)C[C@H](CCCO)CN1C(=O)C(F)(F)F ; CC1(C)C[C@H](CCC=O)CN1C(=O)C(F)(F)F]

2,2,2-Trifluoro-1-[(4S)-4-(3-hydroxypropyl)-2,2-dimethyl-pyrrolidin-1-yl]ethanone (7 g, 26.26 mmol) was dissolved in DCM (100 mL). The solution was cooled in ice water bath. Dess-Martin periodinane (12.895 g, 28.88 mmol) was added in small portions within 1 min. The mixture was stirred under nitrogen (balloon) while the ice bath was removed. After 2 h, a mixed solution of NaS₂O₃ (˜10 g) in saturated aqueous NaHCO₃(50 mL) was added. The mixture was stirred for 30 min. The layers were separated. The DCM layer was further extracted with NaHCO₃(saturated aqueous 50 mL×2), dried over anhydrous Na₂SO₄, filtered and concentrated. The residue oil was purified by silica gel chromatography (Rf: 0.48 3/1 hexanes/EtOAc), using 0-50% EtOAc in hexanes to afford 3-[(3S)-5,5-dimethyl-1-(2,2,2-trifluoroacetyl)pyrrolidin-3-yl]propanal (5.58 g, 80%). ESI-MS m/z calc. 251.1133, found 252.5 (M+1)⁺; Retention time: 3.54 minutes (LC method B).

### Step 3: N-[3-[(3S)-5,5-Dimethyl-1-(2,2,2-trifluoroacetyl)pyrrolidin-3-yl]propylidene]-2-methyl-propane-2-sulfinamide

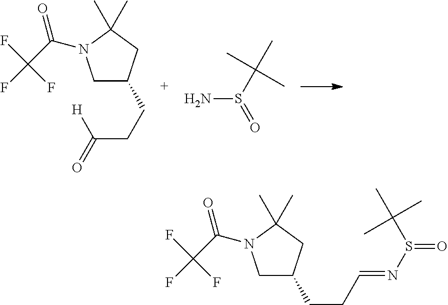

3-[(3S)-5,5-Dimethyl-1-(2,2,2-trifluoroacetyl)pyrrolidin-3-yl]propanal (7.5 g, 28.36 mmol) was dissolved in DCM (70 mL) at rt. 2-methylpropane-2-sulfinamide (3.45 g, 27.896 mmol) was added, followed by magnesium sulfate (18 g, 148.79 mmol) and pyridinium p-toluenesulfonate (358 mg, 1.40 mmol). The mixture was stirred under nitrogen atmosphere for 18 h. It was then filtered through a celite pad, which was washed with more DCM. The combined filtrate was concentrated and the residue was purified by silica gel flash chromatography, using 5-60% EtOAc in hexanes to afford N-[3-[(3S)-5,5-dimethyl-1-(2,2,2-trifluoroacetyl)pyrrolidin-3-yl]propylidene]-2-methyl-propane-2-sulfinamide (9.8 g, 93%). ESI-MS m/z calc. 354.1589, found 355.5 (M+1)⁺; Retention time: 3.12 minutes (LC method B).

### Example 99: Preparation of (14S)-12,12-dimethyl-17-(pyridin-2-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (diastereomer 1, Compound 362) and (14S)-12,12-dimethyl-17-(pyridin-2-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (diastereomer 2, Compound 361)

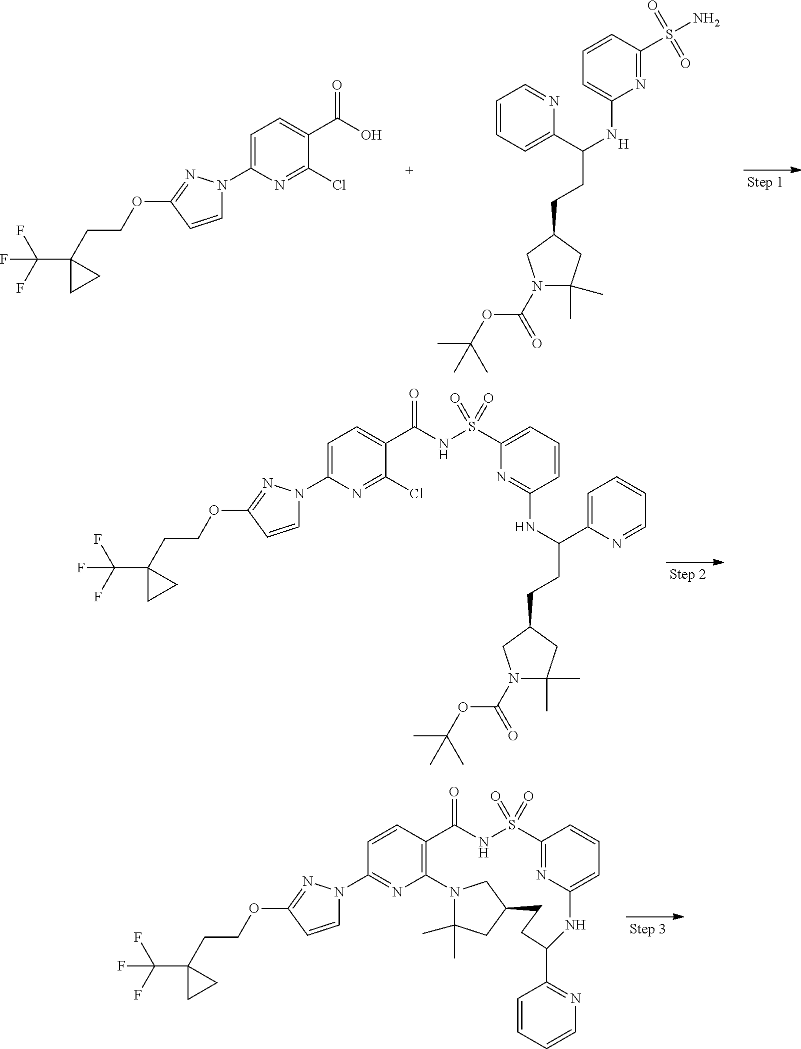

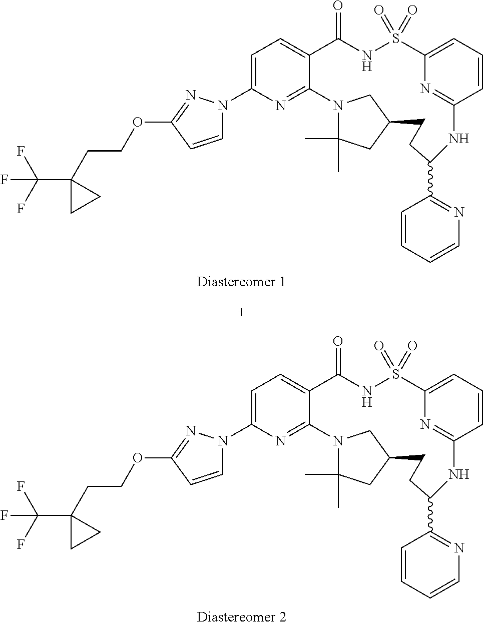 [SMILES: CC1(C)C[C@@H]2CCC(c3ccccn3)Nc3cccc(n3)S(=O)(=O)NC(=O)c3ccc(-n4ccc(OCCC5(C(F)(F)F)CC5)n4)nc3N1C2 ; CC1(C)C[C@@H]2CCC(c3ccccn3)Nc3cccc(n3)S(=O)(=O)NC(=O)c3ccc(-n4ccc(OCCC5(C(F)(F)F)CC5)n4)nc3N1C2]

### Step 1: tert-Butyl (4S)-4-[3-[[6-[[2-chloro-6-[3-[2-[1-(trifluoromethyl)cyclo propyl]ethoxy]pyrazol-1-yl]pyridine-3-carbonyl]sulfamoyl]-2-pyridyl]amino]-3-(2-pyridyl)propyl]-2,2-dimethyl-pyrrolidine-1-carboxylate

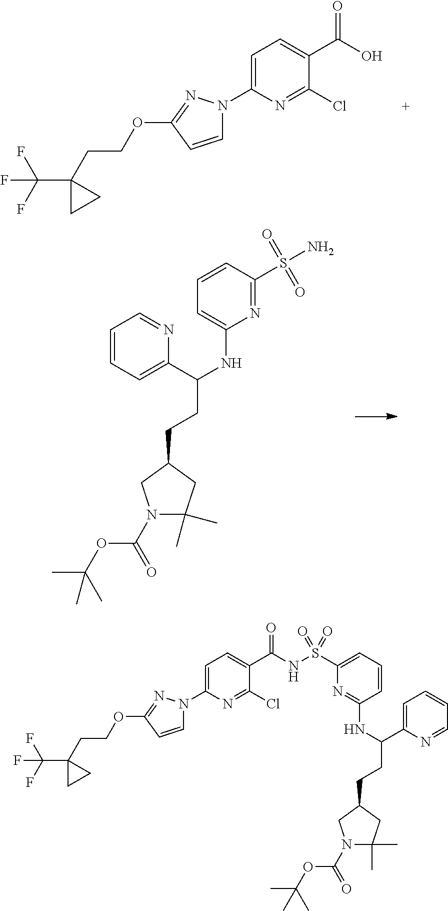 [SMILES: O=C(O)c1ccc(-n2ccc(OCCC3(C(F)(F)F)CC3)n2)nc1Cl ; CC(C)(C)OC(=O)N1C[C@@H](CCC(Nc2cccc(S(N)(=O)=O)n2)c2ccccn2)CC1(C)C ; CC(C)(C)OC(=O)N1C[C@@H](CCC(Nc2cccc(S(=O)(=O)NC(=O)c3ccc(-n4ccc(OCCC5(C(F)(F)F)CC5)n4)nc3Cl)n2)c2ccccn2)CC1(C)C]

To a solution of 2-chloro-6-[3-[2-[1-(trifluoromethyl)cyclopropyl]ethoxy]pyrazol-1-yl]pyridine-3-carboxylic acid (350 mg, 0.9315 mmol) in THF (6 mL) was added CDI (150 mg, 0.9251 mmol) (recrystallized from THF) and the mixture was stirred at rt for 3 h then tert-butyl (4S)-2,2-dimethyl-4-[3-(2-pyridyl)-3-[(6-sulfamoyl-2-pyridyl)amino]propyl]pyrrolidine-1-carboxylate (271 mg, 0.5535 mmol) was added followed by DBU (275 μL, 1.839 mmol) and the resulting mixture was stirred for 16 h at rt. The reaction was diluted with ethyl acetate and washed with a saturated aqueous sodium bicarbonate solution. The organic layer was further washed with 10% citric acid solution followed by brine. The organics were separated, dried over sodium sulfate, evaporated and then purified on silica gel chromatography (40 gram column) using a gradient from 100% hexanes to 100% ethyl acetate followed by a second silica gel column (40 gram column) using a gradient from 100% dichloromethane to 15% methanol in dichloromethane to afford tert-butyl (4S)-4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]-3-(2-pyridyl)propyl]-2,2-dimethyl-pyrrolidine-1-carboxylate (309 mg, 66%) as an off-white solid. ESI-MS m/z calc. 846.29016, found 847.2 (M+1)⁺; Retention time: 1.98 minutes (LC method A).

### Step 2: (14S)-12,12-Dimethyl-17-(pyridin-2-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione

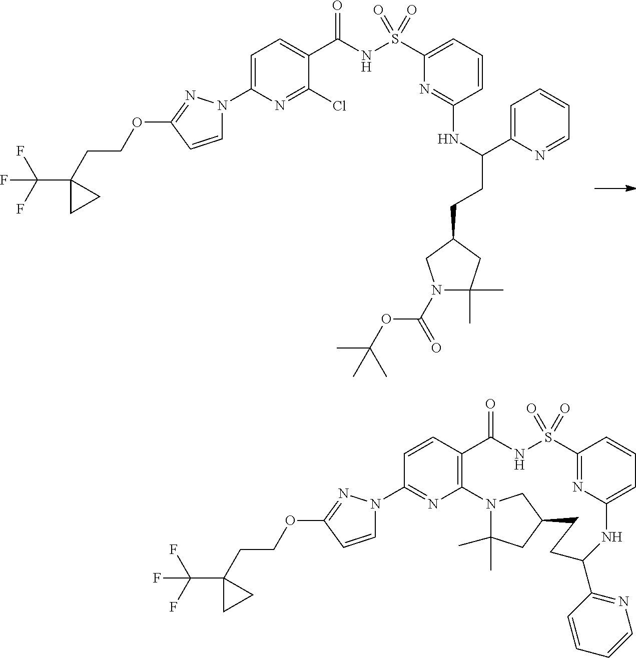 [SMILES: CC(C)(C)OC(=O)N1C[C@@H](CCC(Nc2cccc(S(=O)(=O)NC(=O)c3ccc(-n4ccc(OCCC5(C(F)(F)F)CC5)n4)nc3Cl)n2)c2ccccn2)CC1(C)C ; CC1(C)C[C@@H]2CCC(c3ccccn3)Nc3cccc(n3)S(=O)(=O)NC(=O)c3ccc(-n4ccc(OCCC5(C(F)(F)F)CC5)n4)nc3N1C2]

Stage 1: tert-Butyl (4S)-4-[3-[[64-[2-chloro-6-[3-[2-[1-(trifluoromethyl)cyclopropyl]ethoxy]pyrazol-1-yl]pyridine-3-carbonyl]sulfamoyl]-2-pyridyl]aminol-3-(2-pyridyl)propyl]-2,2-dimethyl-pyrrolidine-1-carboxylate (309 mg, 0.3647 mmol) was dissolved in DCM (14 mL) and to the mixture was added HCl (4M in dioxane) (3.0 mL, 12.00 mmol) and the reaction was stirred at room temperature. After 2 h, the reaction was basified with aqueous sodium carbonate until pH˜8. It was then diluted with ethyl acetate and washed with a saturated aqueous sodium bicarbonate solution. The organic layer was extracted and then further washed with brine. The organics were separated, dried over sodium sulfate, evaporated and then placed on the high vacuum pump for 2 h to afford the intermediate 2-chloro-N-[[6-[[3-[(3S)-5,5-dimethylpyrrolidin-3-yl]-1-(2-pyridyl)propyl]amino]-2-pyridyl]sulfonyl]-6-[3-[2-[1-(trifluoromethyl)cyclopropyl]ethoxy]pyrazol-1-yl]pyridine-3-carboxamide (hydrochloride salt) as an off-white solid. ESI-MS m/z calc. 746.23773, found 747.2 (M+1)⁺; Retention time: 1.4 minutes (LC method A).

Stage 2: Combined material from stage 1 and K₂CO₃ (560 mg, 4.052 mmol), 3 Å molecular sieves and DMSO (14 mL) in a vial, purged with nitrogen, capped, heated to 155° C. and stirred for 18h. Cooled to room temperature and the reaction was diluted with ethyl acetate and washed with a saturated aqueous sodium bicarbonate solution. The organic layer was further washed with brine. The organics were separated, dried over sodium sulfate, evaporated and then purified by silica gel chromatography (40 gram column) using a gradient from 100% hexanes to 100% ethyl acetate to afford (14S)-12,12-dimethyl-17-(pyridin-2-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-tri one (99 mg, 38%). ESI-MS m/z calc. 710.26105, found 711.2 (M+1)⁺; Retention time: 1.81 minutes (LC method A).

### Step 3: (14S)-12,12-Dimethyl-17-(pyridin-2-yl)-8-(3-{2-[1-(trifluoromethyl) cyclopropyl]ethoxy}-1H-pyrazol-1-yl)-2λ⁶-thia-3,9,11,18,23-pentaazatetracyclo [17.3.1.111,14.05,10]tetracosa-1(23),5(10),6,8,19,21-hexaene-2,2,4-trione (diastereomer 1, Compound 362) and (14S)-12,12-dimethyl-17-(pyridin-2-yl)-8-(3-{2-[1-(trifluoromethyl) cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (diastereomer 2, Compound 361)

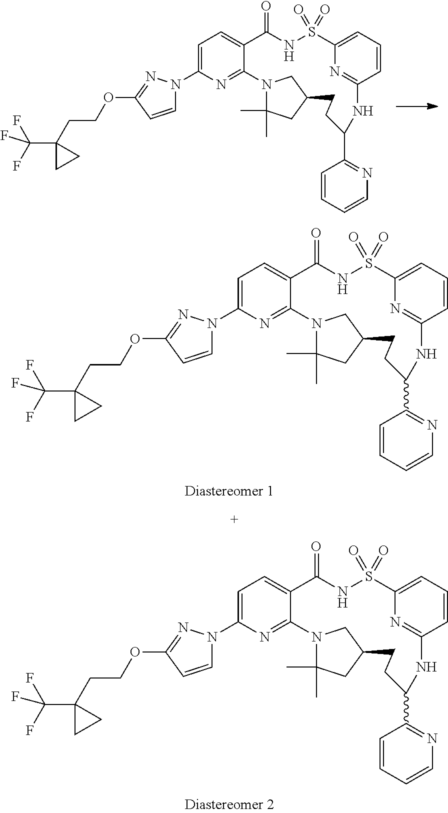

(14S)-12,12-Dimethyl-17-(pyridin-2-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (mixture of two diastereomers, 98 mg, 0.1379 mmol) was subjected to SFC using the following method: ChiralPaK IC (250×21.2 mm, 5 μm) column, 40° C., mobile phase 41% MeOH, 59% CO₂, flow 70 mL/min, concentrations 30 mg/mL in MeOH:DMSO (90:10), injection volume 500 μL, pressure 166 bar, wavelength 278 nm. Two diastereomers were separated:

Diastereomer 1: off-white solid: (14S)-12,12-Dimethyl-17-(pyridin-2-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (44.22 mg, 90%). ¹H NMR (500 MHz, DMSO-d₆) δ 12.58 (s, 1H), 8.52 (d, J=4.9 Hz, 1H), 8.23 (d, J=2.8 Hz, 1H), 7.84 (d, J=8.2 Hz, 1H), 7.69 (dt, J=47.2, 7.8 Hz, 3H), 7.44 (d, J=7.9 Hz, 1H), 7.23 (dd, J=7.6, 4.9 Hz, 1H), 7.10 (d, J=7.1 Hz, 1H), 6.91 (dd, J=24.4, 8.3 Hz, 2H), 6.12 (d, J=2.8 Hz, 1H), 5.26 (s, 1H), 4.32 (t, J=7.0 Hz, 2H), 2.80 (s, 1H), 2.24 (s, 1H), 2.08 (t, J=7.2 Hz, 2H), 2.00-1.85 (m, 3H), 1.75 (d, J=14.4 Hz, 1H), 1.64 (s, 4H), 1.53 (s, 3H), 1.48 (d, J=12.9 Hz, 1H), 1.23 (s, 1H), 0.96 (d, J=4.7 Hz, 2H), 0.87 (d, J=18.8 Hz, 2H). ESI-MS m/z calc. 710.26105, found 711.2 (M+1)⁺; Retention time: 1.8 minute (LC method A).

Diastereomer 2: off-white solid: (14S)-12,12-Dimethyl-17-(pyridin-2-yl)-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (42.62 mg, 87%). ESI-MS m/z calc. 710.26105, found 711.2 (M+1)⁺; Retention time: 1.83 minutes (LC method A).

### Example 100: Preparation of (14S)-8-13-(2-{dispiro[2.0.2.11heptan-7-yl}ethoxy)-1H-pyrazol-1-yl]-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 1, Compound 356), and (14S)-8-[3-(2-{dispiro[2.0.2.1]heptan-7-yl}ethoxy)-1H-pyrazol-1-yl]-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 2, Compound 355)

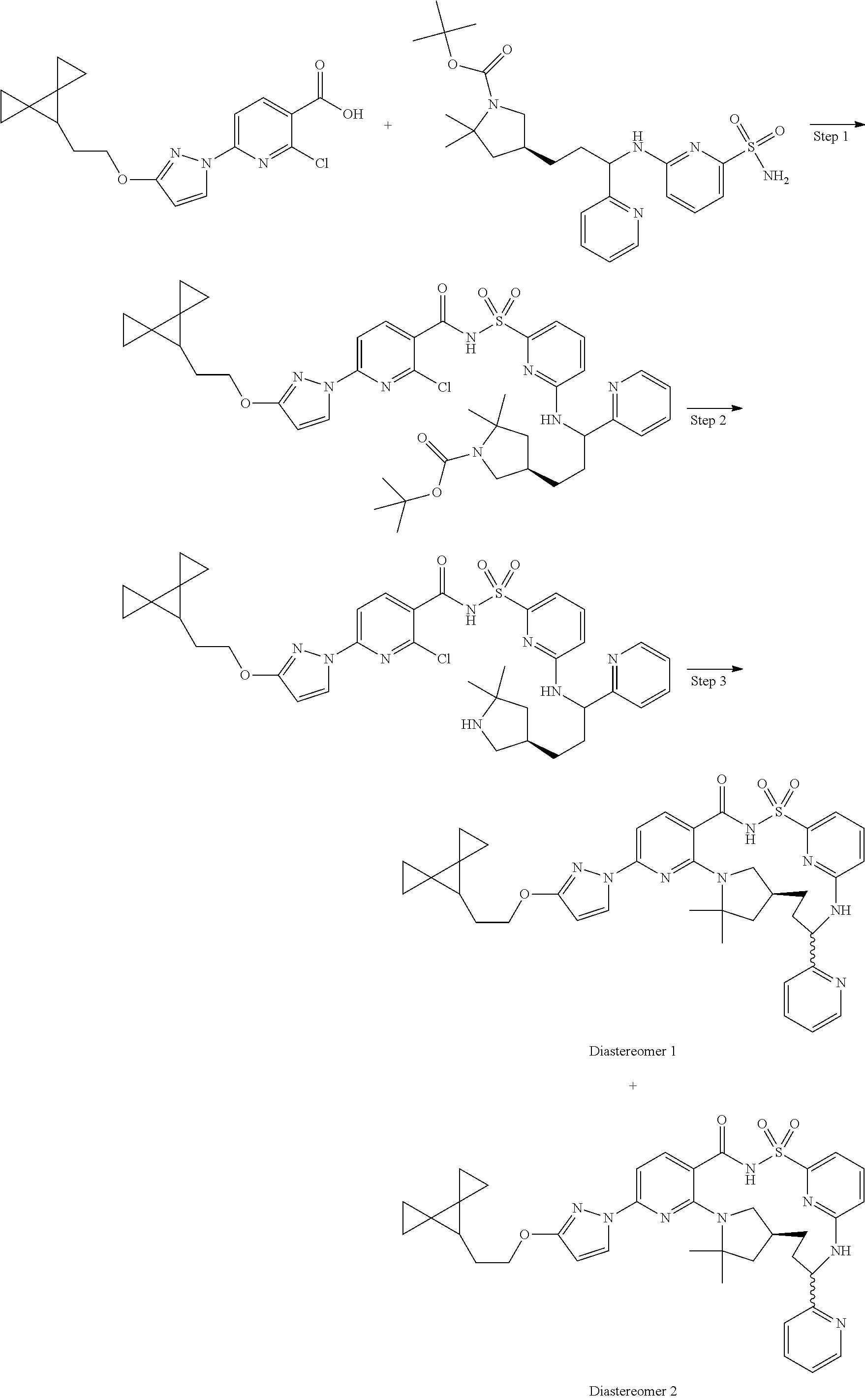

### Step 1: tert-Butyl (4R)-4-[3-[[6-[[2-chloro-6-[3-(2-dispiro[2.0.2.1]heptan-7-ylethoxy)pyrazol-1-yl]pyridine-3-carbonyl]sulfamoyl]-2-pyridyl]amino]-3-(2-pyridyl)propyl]-2,2-dimethyl-pyrrolidine-1-carboxylate

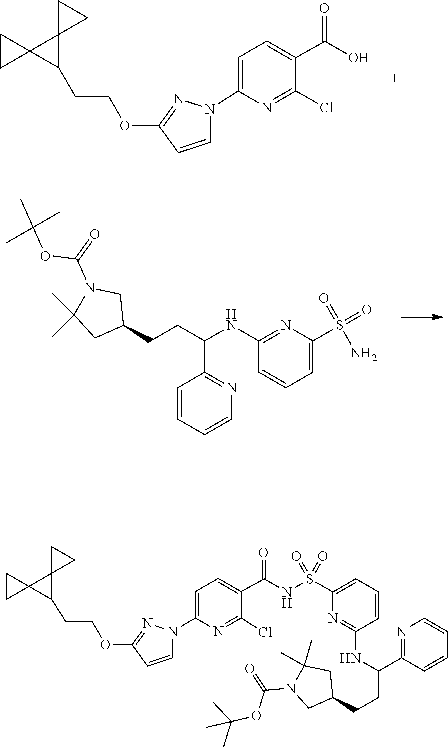 [SMILES: O=C(O)c1ccc(-n2ccc(OCCC3C4(CC4)C34CC4)n2)nc1Cl ; CC(C)(C)OC(=O)N1C[C@@H](CCC(Nc2cccc(S(N)(=O)=O)n2)c2ccccn2)CC1(C)C ; CC(C)(C)OC(=O)N1C[C@H](CCC(Nc2cccc(S(=O)(=O)NC(=O)c3ccc(-n4ccc(OCCC5C6(CC6)C56CC6)n4)nc3Cl)n2)c2ccccn2)CC1(C)C]

To a solution of 2-chloro-6-[3-(2-dispiro[2.0.2.1]heptan-7-ylethoxy)pyrazol-1-yl]pyridine-3-carboxylic acid (300 mg, 0.8338 mmol) in THF (1.80 mL) was added CDI (168.8 mg, 1.041 mmol) (recrystallized from THF) and the mixture was stirred at rt for 2h then tert-butyl (4S)-2,2-dimethyl-4-[3-(2-pyridyl)-3-[(6-sulfamoyl-2-pyridyl)amino]propyl]pyrrolidine-1-carboxylate (428.7 mg, 0.8756 mmol) was added as a solution in THF (1.80 mL) followed by DBU (398.6 mg, 2.618 mmol) and the resulting mixture was stirred for 3 h at room temperature. The reaction was diluted with water and EtOAc then HCl (932.2 μL of 6 M, 5.593 mmol) was added. The layers were separated and the organic layer was washed with water (1×) and brine (1×) then dried over sodium sulfate and concentrated to a white foam which was filtered and purified using a reverse phase HPLC-MS method using a LuNa C₁₈ (2) column (75×30 mm, 5 μm particle size) sold by Phenomenex (pn: 00C-4252-U0-AX), and a dual gradient run from 1-99% mobile phase B over 15.0 minutes (mobile phase A=H₂O (5 mM HCl), mobile phase B=acetonitrile, flow rate=50 mL/min, injection volume=950 μL and column temperature=25° C.) giving tert-butyl (4R)-4-[3-[[6-[[2-chloro-6-[3-(2-dispiro[2.0.2.1]heptan-7-ylethoxy)pyrazol-1-yl]pyridine-3-carbonyl]sulfamoyl]-2-pyridyl]amino]-3-(2-pyridyl)propyl]-2,2-dimethyl-pyrrolidine-1-carboxylate (330 mg, 48%). ESI-MS m/z calc. 830.3341, found 831.8 (M+1)⁺; Retention time: 0.82 minutes (LC method D).

### Step 2: 2-chloro-N-[[6-[[3-[(3R)-5,5-dimethylpyrrolidin-3-yl]-1-(2-pyridyl)propyl]amino]-2-pyridyl]sulfonyl]-6-[3-(2-dispiro[2.0.2.1]heptan-7-ylethoxy)pyrazol-1-yl]pyridine-3-carboxamide

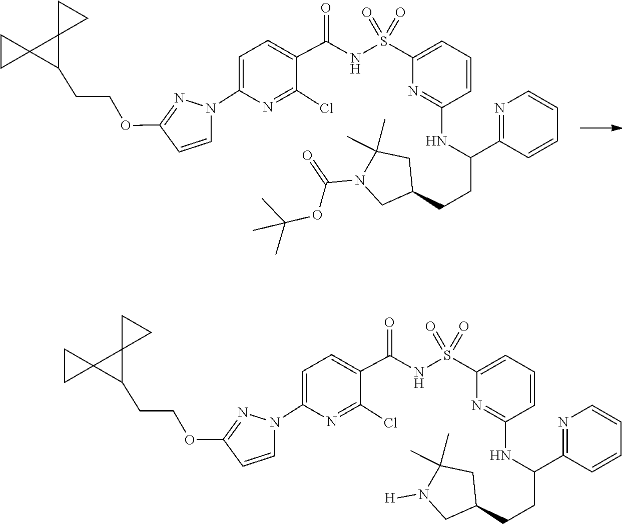 [SMILES: CC(C)(C)OC(=O)N1C[C@H](CCC(Nc2cccc(S(=O)(=O)NC(=O)c3ccc(-n4ccc(OCCC5C6(CC6)C56CC6)n4)nc3Cl)n2)c2ccccn2)CC1(C)C ; CC1(C)C[C@@H](CCC(Nc2cccc(S(=O)(=O)NC(=O)c3ccc(-n4ccc(OCCC5C6(CC6)C56CC6)n4)nc3Cl)n2)c2ccccn2)CN1]

tert-Butyl (4R)-4-[3-[[6-[[2-chloro-6-[3-(2-dispiro[2.0.2.1]heptan-7-ylethoxy)pyrazol-1-yl]pyridine-3-carbonyl]sulfamoyl]-2-pyridyl]amino]-3-(2-pyridyl)propyl]-2,2-dimethyl-pyrrolidine-1-carboxylate (330 mg, 0.3969 mmol) was dissolved in DCM (1.44 mL) and to the mixture was added TFA (1.22 mL, 15.89 mmol) and the mixture was stirred at room temperature for 3 h. Concentrated mixture to dryness under reduced pressure, added 1 mL of toluene and removed by rotary evaporation (45° C. water bath). Again added 1 mL of toluene and removed by rotary evaporation (45° C. water bath) then dried under vacuum to give 2-chloro-N-[[6-[[3-[(3R)-5,5-dimethylpyrrolidin-3-yl]-1-(2-pyridyl)propyl]amino]-2-pyridyl]sulfonyl]-6-[3-(2-dispiro[2.0.2.1]heptan-7-ylethoxy)pyrazol-1-yl]pyridine-3-carboxamide (trifluoroacetate salt) (335.5 mg, 100%) ESI-MS m/z calc. 730.2817, found 731.7 (M+1)⁺; Retention time: 0.57 minutes as a white solid (LC method D).

### Step 3: (14S)-8-13-(2-{dispiro[2.0.2.11heptan-7-yl}ethoxy)-1H-pyrazol-1-yl]-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 1, Compound 356), and (14S)-8-[3-(2-{dispiro[2.0.2.1]heptan-7-yl}ethoxy)-1H-pyrazol-1-yl]-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 2, Compound 355)

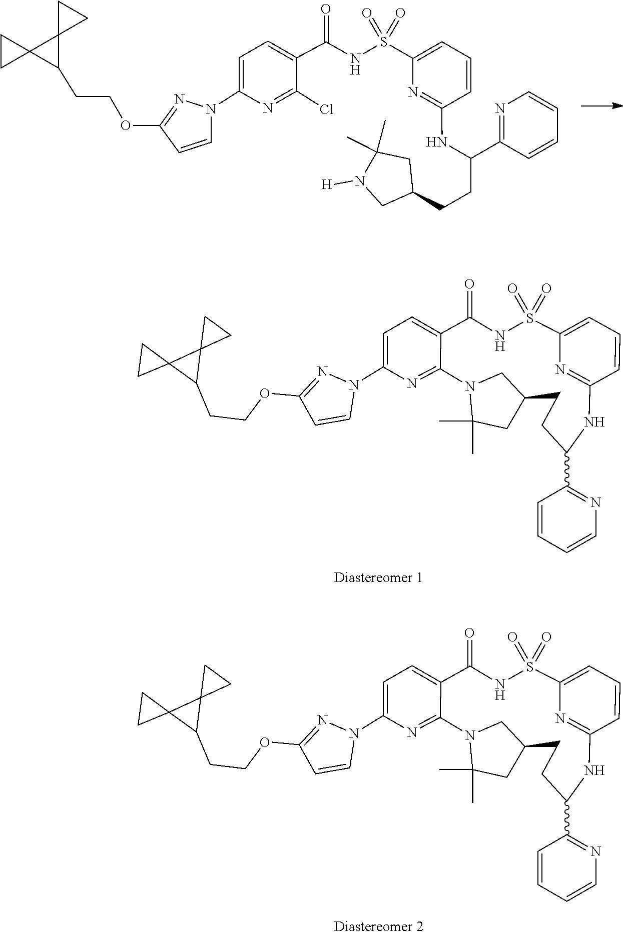 [SMILES: CC1(C)C[C@@H](CCC(Nc2cccc(S(=O)(=O)NC(=O)c3ccc(-n4ccc(OCCC5C6(CC6)C56CC6)n4)nc3Cl)n2)c2ccccn2)CN1 ; CC1(C)C[C@@H]2CCC(c3ccccn3)Nc3cccc(n3)S(=O)(=O)NC(=O)c3ccc(-n4ccc(OCCC5C6(CC6)C56CC6)n4)nc3N1C2 ; CC1(C)C[C@@H]2CCC(c3ccccn3)Nc3cccc(n3)S(=O)(=O)NC(=O)c3ccc(-n4ccc(OCCC5C6(CC6)C56CC6)n4)nc3N1C2]

To a solution of 2-chloro-N-[[6-[[3-[(3R)-5,5-dimethylpyrrolidin-3-yl]-1-(2-pyridyl)propyl]amino]-2-pyridyl]sulfonyl]-6-[3-(2-dispiro[2.0.2.1]heptan-7-ylethoxy)pyrazol-1-yl]pyridine-3-carboxamide (trifluoroacetate salt) (335.5 mg, 0.3969 mmol) in NMP (20 mL) was added potassium carbonate (384.1 mg, 2.779 mmol). The mixture was purged with nitrogen for 5 min. The mixture was heated at 165° C. for 16h. The mixture was cooled to room temperature, diluted with 1N HCl and extracted with EtOAc (1×). The organic phase was washed with brine (1×), dried (sodium sulfate), filtered and concentrated to a brown oil which was filtered and purified using a reverse phase HPLC-MS method using a LuNa C₁₈ (2) column (75×30 mm, 5 μm particle size) sold by Phenomenex (pn: 00C-4252-U0-AX), and a dual gradient run from 50-99% mobile phase B over 15.0 minutes (mobile phase A=H₂O (5 mM HCl), mobile phase B=acetonitrile, flow rate=50 mL/min, injection volume=950 μL and column temperature=25° C.) giving as the first diastereomer to elute, (14S)-8-[3-(2-{dispiro[2.0.2.1]heptan-7-yl}ethoxy)-1H-pyrazol-1-yl]-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (110 mg, 80%) as a white solid. ¹H NMR (500 MHz, DMSO-d₆) δ 12.47 (s, 1H), 8.72 (d, J=5.3 Hz, 1H), 8.20 (d, J=2.8 Hz, 2H), 7.99 (s, 2H), 7.70 (t, J=7.8 Hz, 3H), 7.30 (d, J=7.2 Hz, 1H), 6.95 (d, J=8.4 Hz, 1H), 6.91 (d, J=8.2 Hz, 1H), 6.08 (d, J 2.7 Hz, 1H), 5.16 (s, 1H), 4.21 (t, J=6.7 Hz, 2H), 3.23 (s, 1H), 2.94 (s, 1H), 2.34 (s, 1H), 2.19 (s, 1H), 1.91 (dd, J=12.2, 5.7 Hz, 1H), 1.81 (q, J=6.7 Hz, 3H), 1.60 (s, 4H), 1.55 (s, 4H), 1.47 (s, 1H), 1.19 (s, 1H), 0.83 (d, J=5.7 Hz, 4H), 0.67-0.60 (m, 2H), 0.50 (d, J=9.0 Hz, 2H). ESI-MS m/z calc. 694.305, found 695.7 (M+1)⁺; Retention time: 1.98 minutes (LC method A), and as the second diastereomer to elute, (14S)-8-[3-(2-{dispiro[2.0.2.1]heptan-7-yl}ethoxy)-1H-pyrazol-1-yl]-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (70 mg, 51%) as a white solid. ¹H NMR (500 MHz, DMSO-d₆) δ 12.55 (s, 1H), 8.77 (d, J=5.3 Hz, 1H), 8.33 (s, 1H), 8.23 (d, J=2.7 Hz, 1H), 8.08-8.00 (m, 1H), 7.95 (s, 1H), 7.86 (d, J=8.2 Hz, 1H), 7.73 (t, J=7.9 Hz, 2H), 7.19 (d, J=7.2 Hz, 1H), 6.98 (d, J=8.5 Hz, 1H), 6.94 (d, J=8.2 Hz, 1H), 6.10 (d, J=2.7 Hz, 1H), 5.48 (s, 1H), 4.22 (t, J=6.6 Hz, 2H), 3.42 (s, 1H), 2.74 (t, J=10.3 Hz, 1H), 2.42 (s, 1H), 2.04 (s, 2H), 1.90 (dd, J=11.7, 5.2 Hz, 1H), 1.82 (q, J=6.5 Hz, 2H), 1.78 (s, 1H), 1.66 (s, 3H), 1.61 (d, J=12.5 Hz, 1H), 1.54 (s, 3H), 1.53-1.45 (m, 2H), 0.84 (q, J=4.1, 3.6 Hz, 4H), 0.65 (dd, J=8.4, 4.3 Hz, 2H), 0.51 (dd, J=8.8, 4.2 Hz, 2H). ESI-MS m/z calc. 694.305, found 695.7 (M+1)⁺; Retention time: 2.0 minutes (LC method A).

### Example 102: Preparation of (14S)-8-[3-fluoro-5-(2-methylpropoxy)phenyl]-12,12-dimethyl-17-(pyridin-2-yl)-2λ⁶-thia-3,9,11,18,23-pentaazatetracyclo[17.3.1.111,14.05,10]tetracosa-1(22),5(10),6,8,19(23),20-hexaene-2,2,4-trione, (diastereomer 1, Compound 352) and (14S)-8-[3-fluoro-5-(2-methylpropoxy)phenyl]-12,12-dimethyl-17-(pyridin-2-yl)-2λ⁶-thia-3,9,11,18,23-pentaazatetracyclo[17.3.1.111,14.05,10]tetracosa-1(22),5(10),6,8,19(23),20-hexaene-2,2,4-trione (diastereomer 2, Compound 351)

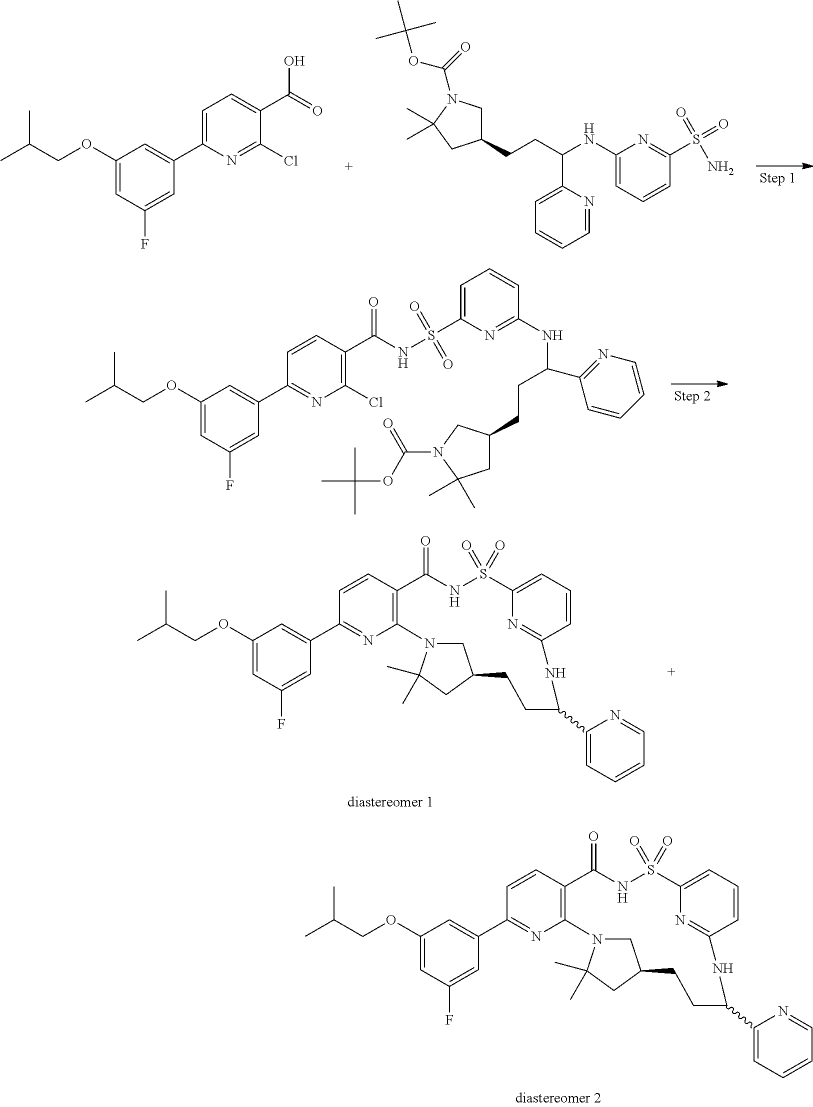

### Step 1: tert-Butyl (4S)-4-[3-[[6-[[2-chloro-6-(3-fluoro-5-isobutoxy-phenyl)pyridine-3-carbonyl]sulfamoyl]-2-pyridyl]amino]-3-(2-pyridyl)propyl]-2,2-dimethyl-pyrrolidine-1-carboxylate

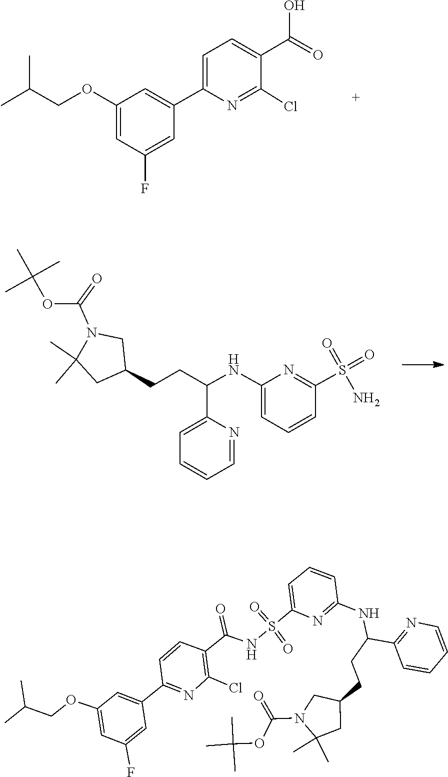 [SMILES: CC(C)COc1cc(F)cc(-c2ccc(C(=O)O)c(Cl)n2)c1 ; CC(C)(C)OC(=O)N1C[C@@H](CCC(Nc2cccc(S(N)(=O)=O)n2)c2ccccn2)CC1(C)C ; CC(C)COc1cc(F)cc(-c2ccc(C(=O)NS(=O)(=O)c3cccc(NC(CC[C@@H]4CN(C(=O)OC(C)(C)C)C(C)(C)C4)c4ccccn4)n3)c(Cl)n2)c1]

To a solution of 2-chloro-6-(3-fluoro-5-isobutoxy-phenyl)pyridine-3-carboxylic acid (143 mg, 0.4417 mmol) in THF (5 mL) was added CDI (78 mg, 0.4810 mmol) and the mixture was stirred at rt for 3 h then tert-butyl (4S)-2,2-dimethyl-4-[3-(2-pyridyl)-3-[(6-sulfamoyl-2-pyridyl)amino]propyl]pyrrolidine-1-carboxylate (170 mg, 0.3472 mmol) was added followed by DBU (200 μL, 1.337 mmol) and the resulting mixture was stirred for 2 h at room temperature. The reaction was quenched with 1;1 mixture of saturated ammonium chloride and brine solutions, then extracted with ethyl acetate. The combined organic layers washed with brine, dried over sodium sulfate, filtered and evaporated. The resultant residue was purified by reverse phase HPLC-MS method using a dual gradient run from 30-99% mobile phase B over 15.0 minutes. Mobile phase A=H₂O (5 mM HCl). Mobile phase B=CH₃CN to afford tert-butyl (4S)-4-[3-[[6-[[2-chloro-6-(3-fluoro-5-isobutoxy-phenyl)pyridine-3-carbonyl]sulfamoyl]-2-pyridyl]amino]-3-(2-pyridyl)propyl]-2,2-dimethyl-pyrrolidine-1-carboxylate (152 mg, 55%) ESI-MS m/z calc. 794.30286, found 795.4 (M+1)⁺; Retention time: 0.63 minutes as off white solid (LC method G).

### Step 2: (14S)-8-[3-fluoro-5-(2-methylpropoxy)phenyl]-12,12-dimethyl-17-(pyridin-2-yl)-2λ⁶-thia-3,9,11,18,23-pentaazatetracyclo[17.3.1.111,14.05,10]tetracosa-1(22),5(10),6,8,19(23),20-hexaene-2,2,4-trione, (diastereomer 1, Compound 352) and (14S)-8-[3-fluoro-5-(2-methylpropoxy)phenyl]-12,12-dimethyl-17-(pyridin-2-yl)-2λ⁶-thia-3,9,11,18,23-pentaazatetracyclo[17.3.1.111,14.05,10]tetracosa-1(22),5(10),6,8,19(23),20-hexaene-2,2,4-trione (diastereomer 2, Compound 351)

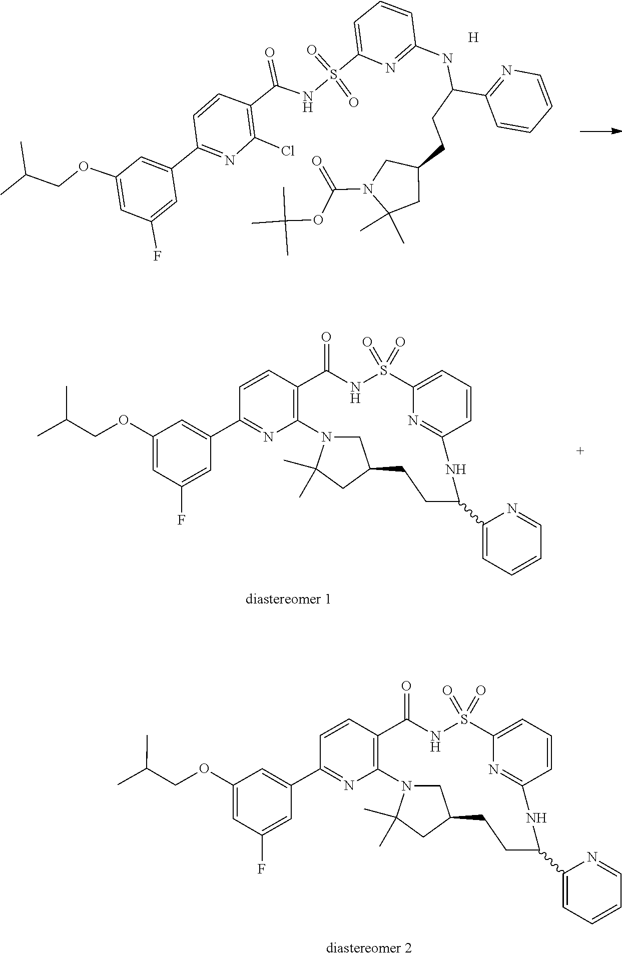

To a solution of tert-butyl (4S)-4-[3-[[6-[[2-chloro-6-(3-fluoro-5-isobutoxy-phenyl)pyridine-3-carbonyl]sulfamoyl]-2-pyridyl]amino]-3-(2-pyridyl)propyl]-2,2-dimethyl-pyrrolidine-1-carboxylate (152 mg, 0.1911 mmol) in (1:4 TFA-DCM premixed solution) TFA (150 μL, 1.947 mmol) and DCM (450 μL) was stirred at rt and solvent removed. The residue was dissolved in NMP (5 mL) and potassium carbonate (331 mg, 2.395 mmol) was added. The mixture was heated at 140° C. for 16h. The mixture was cooled to room temperature and the reaction mixture was filtered through Whatman filter disc (puradisc 25 TF) and the filtrate was purified by a reverse phase HPLC-MS method using a dual gradient run from 30-99% mobile phase B over 15 minutes (mobile phase A=H₂O (5 mM HCl). Mobile phase B=CH₃CN), giving as eluting diastereomer 1 (14S)-8-[3-fluoro-5-(2-methylpropoxy)phenyl]-12,12-dimethyl-17-(pyridin-2-yl)-2λ⁶-thia-3,9,11,18,23-pentaazatetracyclo[17.3.1.111,14.05,10]tetracosa-1(22),5(10),6,8,19(23),20-hexaene-2,2,4-trione (11.6 mg, 18%). ¹H NMR (500 MHz, DMSO-d₆) δ 12.66 (s, 1H), 8.74-8.70 (m, 1H), 8.24 (s, 1H), 7.99 (s, 2H), 7.72 (t, J=7.9 Hz, 2H), 7.62 (d, J=7.6 Hz, 1H), 7.48 (d, J=2.2 Hz, 1H), 7.45-7.38 (m, 1H), 7.33 (d, J=7.1 Hz, 1H), 7.27 (d, J=7.9 Hz, 1H), 6.97 (d, J=8.4 Hz, 1H), 6.89 (dt, J=10.7, 2.3 Hz, 1H), 5.09 (s, 1H), 3.84 (d, J=6.7 Hz, 2H), 3.31 (t, J=7.1 Hz, 1H), 2.86 (s, 1H), 2.34 (s, 1H), 2.17 (d, J=9.3 Hz, 1H), 2.05 (dp, J=13.3, 6.7 Hz, 1H), 1.91 (dd, J=11.9, 5.7 Hz, 1H), 1.77 (s, 1H), 1.64 (s, 3H), 1.59 (s, 3H), 1.57-1.47 (m, 1H), 1.27-1.06 (m, 1H), 0.99 (d, J=6.7 Hz, 7H). ESI-MS m/z calc. 658.27374, found 659.4 (M+1)⁺; Retention time: 2.95 minutes (LC method A), and as eluting diastereomer 2 (14S)-8-[3-fluoro-5-(2-methylpropoxy)phenyl]-12,12-dimethyl-17-(pyridin-2-yl)-2λ⁶-thia-3,9,11,18,23-pentaazatetracyclo[17.3.1.111,14.05,10]tetracosa-1(22),5(10),6,8,19(23),20-hexaene-2,2,4-trione (19.8 mg, 31%). ¹H NMR (500 MHz, DMSO-d₆) δ 12.59 (s, 1H), 8.72 (d, J=5.3 Hz, 1H), 8.21 (s, 1H), 7.95 (d, J=8.8 Hz, 1H), 7.85 (s, 1H), 7.80 (s, 1H), 7.72 (dd, J=8.5, 7.2 Hz, 1H), 7.64 (s, 1H), 7.51 (t, J=1.8 Hz, 1H), 7.43 (dt, J=9.8, 2.0 Hz, 1H), 7.30 (d, J=8.0 Hz, 1H), 7.19 (d, J=7.2 Hz, 1H), 6.97 (d, J=8.4 Hz, 1H), 6.91 (dt, J=10.7, 2.3 Hz, 1H), 5.46 (d, J=10.9 Hz, 1H), 3.85 (d, J=6.6 Hz, 2H), 3.39 (t, J=8.6 Hz, 1H), 2.76 (t, J=10.3 Hz, 1H), 2.37 (s, 1H), 2.13-1.96 (m, 2H), 1.91 (dd, J=11.8, 5.3 Hz, 1H), 1.80 (d, J=13.0 Hz, 1H), 1.69 (s, 3H), 1.65 (d, J=12.4 Hz, 1H), 1.61 (s, 3H), 1.56-1.47 (m, 1H), 1.00 (d, J=6.6 Hz, 7H). ESI-MS m/z calc. 658.27374, found 659.4 (M+1)⁺; Retention time: 2.95 minutes (LC method A).

### Example 103: Preparation of 3-(3,3-dimethylbutoxy)-1H-pyrazole

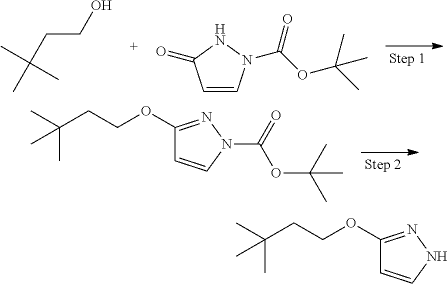

### Step 1: tert-Butyl 3-(3,3-dimethylbutoxy)pyrazole-1-carboxylate

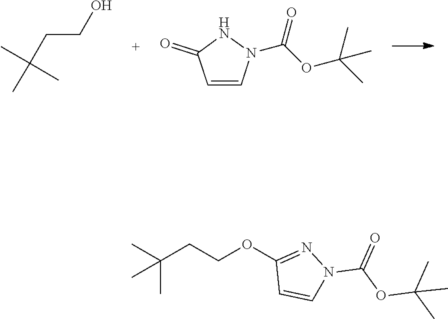

A 5000 mL 3 neck round bottom flask as fitted with a mechanical stirrer, a heating mantle, a J-Kem temperature probe/controller, an addition funnel, a water cooled reflux condenser and a nitrogen inlet/outlet. The vessel was charged under a nitrogen atmosphere with tert-butyl 5-oxo-1H-pyrazole-2-carboxylate (100 g, 0.5429 mol) and tetrahydrofuran (1200 mL, 12 mL/g) which provided a clear pale yellow solution. Stirring was commenced and the pot temperature was recorded at 19° C. The vessel was then charged with 3,3-dimethylbutan-1-ol (52.6 g, 0.5972 mol) added as a solid in one portion followed by triphenylphosphine (156.6 g, 0.5972 mol) added as a solid in one portion. The resulting clear pale yellow solution was then treated with diisopropyl azodicarboxylate (clear reddish-orange liquid) (117.6 mL, 0.5972 mol) added neat dropwise over 1 hour which resulted in a gradual exotherm to 40° C. and a clear light amber solution. The reaction mixture was then heated to a pot temperature of 50° C. and the condition was maintained for 2 hours when analysis by LC/MS indicated complete consumption of the starting material. The clear amber reaction mixture was concentrated under reduced pressure and the resulting clear dark amber oil was suspended in toluene (800 mL) and stirred at rt for 1 hour during which time a solid (triphenylphosphine oxide MW=278.28) precipitated. The thick slurry was filtered through a glass frit Buchner funnel and the filter cake was displacement washed with toluene (150 mL) and then pulled for 30 minutes. The off-white solid filter cake was labeled E28965-202-FC-TPO and was consistent by LC/MS for triphenylphosphine oxide. The clear amber filtrate was concentrated under reduced pressure to provide a clear amber oil. The material was purified by silica gel column flash chromatography (solid load on Celite, 1.5 kg silica RediSep column) eluting with a gradient of 100% hexane to 20% EtOAc in hexane collecting 450 mL fractions. The product elutes around 5% EtOAc in hexane. The desired fractions were combined and concentrated under reduced pressure to provide a clear colorless oil which solidified upon standing to provide a white solid (110 g, 0.410 mol, 75% yield) as the desired product tert-butyl 3-(3,3-dimethylbutoxy)pyrazole-1-carboxylate. ESI-MS m/z calc. 268.17868, found 279.0 (M+1)⁺; Retention time: 1.26 minutes (LC method A).

### Step 2: 3-(3,3-dimethylbutoxy)-1H-pyrazole

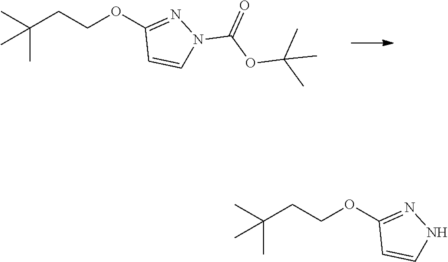

A 5000 mL 3 neck round bottom flask was fitted with a mechanical stirrer, a heating mantle, a J-Kem temperature probe, a water-cooled reflux condenser, an addition funnel and a nitrogen inlet/outlet. The vessel was charged under a nitrogen atmosphere with tert-butyl 3-(3,3-dimethylbutoxy)pyrazole-1-carboxylate (110 g, 0.4099 mol), dichloromethane (440 mL, 4 mL/g) and methyl alcohol (440 mL, 4 mL/g) which provided a clear colorless solution. Stirring was commenced and the pot temperature was recorded at 19° C. The addition funnel was charged with 4 Molar HCl in 1,4-dioxane (307.5 mL, 1.230 mol) which was subsequently added dropwise over 1 hour which resulted in a gradual exotherm to 30° C. The resulting clear pale yellow solution was heated to a pot temperature of 45° C. and the condition was maintained for 1 hour when analysis by LC/MS indicated reaction completion. The reaction mixture was allowed to cool to rt and then concentrated under reduced pressure. The remaining residue was dissolved in tert-butyl methyl ether (1200 mL) and then transferred to a separatory funnel and partitioned with 2 Molar sodium hydroxide solution (615 mL, 1.230 mol). The organic was removed and the residual aqueous was extracted with tert-butyl methyl ether (2×200 mL). The combined organic was washed with saturated sodium chloride solution (500 mL), dried over sodium sulfate (300 g) and then filtered through a glass frit Buchner funnel. The clear pale yellow filtrate was concentrated under reduced pressure to provide a clear light yellow oil (67 g, 0.398 mol, 97% yield) as the desired product 3-(3,3-dimethylbutoxy)-1H-pyrazole. ¹H NMR (400 MHz, DMSO-d₆) δ 7.50 (d, J=2.4 Hz, 1H), 5.64 (d, J=2.3 Hz, 1H), 4.09 (dd, J=7.7, 7.0 Hz, 2H), 1.71-1.52 (m, 2H), 0.94 (s, 9H). ESI-MS m/z calc. 168.12627, found 169.1 (M+1)⁺; Retention time: 1.26 minutes (LC method A).

The pyrazole reagents in the following table were prepared in a manner analogous to that described above:

| | | Retention | | | |
|---|---|---|---|---|---|
| | | Time | Exact | | Lcms |
| Structure | NMR | (min) | Mass | M + 1 | Method |
| 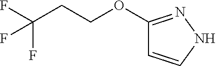 [SMILES: FC(F)(F)CCOc1cc[nH]n1] | | 0.35 | 180.05 | 180.9 | LC method A |
| 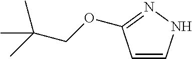 | ¹H NMR (400 MHz, Chloroform-d) δ 7.36 (d, J = 2.4 Hz, 1H), 5.74 (d, J = 2.5 Hz, 1H),3.81 (s, 2H), 1.02 (s, 9H). | 1.13 | 154.11 | 155.3 | LC method A |

### Example 104: Preparation of 3-((1-Methylcyclopropyl)methoxy)-1H-pyrazole

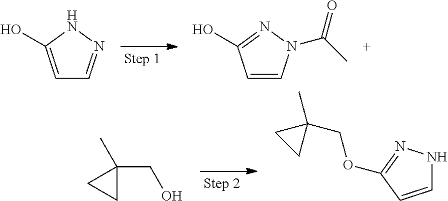

### Step 1: 1-(3-hydroxypyrazol-1-yl)ethenone

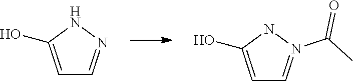

A 100 mL round bottom flask equipped with a stirbar and a condenser was charged with 1H-pyrazol-5-ol (4.97 g, 59.11 mmol) and pyridine (25 mL, 309.1 mmol). The mixture was stirred at 95° C. A solution of acetic anhydride (5.6 mL, 59.35 mmol) in pyridine (10 mL, 123.6 mmol) was added dropwise over a period of 3 minutes. The mixture was then stirred at 95° C. for an additional three hours. The solvents were removed under reduced pressure. The solid residue was triturated in 40 mL of diethyl ether, filtered, washed with diethyl ether and dried to give 1-(3-hydroxypyrazol-1-yl)ethanone (6.96 g, 93%). ¹H NMR (400 MHz, DMSO-d₆) δ 10.96 (s, 1H), 8.13 (d, J=3.0 Hz, 1H), 6.01 (d, J=3.0 Hz, 1H), 2.48 (s, 3H).

### Step 2: 3-((1-Methylcyclopropyl)methoxy)-1H-pyrazole

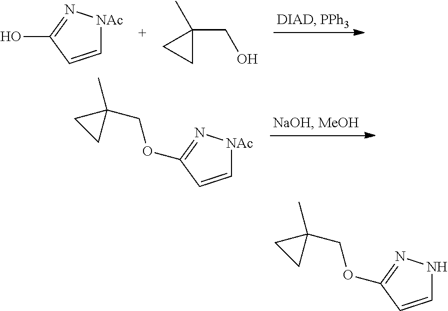

1-(3-Hydroxy-1H-pyrazol-1-yl)ethan-1-one (1.16 g, 9.0 mmol), (1-methylcyclopropyl)methanol (0.80 g, 9.3 mmol) and PPh₃ (2.67 g, 10.1 mmol) were mixed in 20 mL of THF and cooled to 0° C. DIAD (2 mL, 10.1 mmol) was added dropwise. The mixture was allowed to reach room temperature overnight. EtOAc (100 mL) was added and the solution was washed with saturated aqueous NaHCO₃ and brine, dried over Na₂SO₄ and concentrated. The residue was purified by means of column chromatography (silica; heptanes/EtOAc 9:1) to afford 1.1 gram of product contaminated with 22% Ph₃PO and 400 mg pure 1-(3-((1-methylcyclopropyl)methoxy)-1H-pyrazol-1-yl)ethan-1-one as a white solid (combined yield 1.5 g, max 69%). ¹H NMR (CDCl₃, 300 MHz): δ 0.43 (m, 2H); 0.56 (m, 2H); 1.21 (s, 3H); 2.56 (s, 3H); 4.00 (s, 2H); 5.97 (d, 1H); 8.04 (d, 1H).

1-(3-((1-Methylcyclopropyl)methoxy)-1H-pyrazol-1-yl)ethan-1-one (1.5 g, max 6.2 mmol) was dissolved in MeOH (20 mL), 6N aqueous NaOH (1.27 mL, 7.6 mmol) was added and the mixture was stirred overnight at room temperature. The solution was concentrated, stripped with heptanes and coated on silica. The residue was purified by means of column chromatography (silica; heptanes/EtOAc 9:1->1:1) affording 3-((1-methylcyclopropyl)methoxy)-1H-pyrazole (790 mg, 84%) as a colorless oil. ¹H NMR (CDCl₃, 300 MHz): δ 0.41 (m, 2H); 0.54 (m, 2H); 1.22 (s, 3H); 3.92 (s, 2H); 5.73 (d, 1H); 7.34 (d, 1H).

### Example 105: Preparation of 3-(Cyclopropylmethoxy)-1H-pyrazole

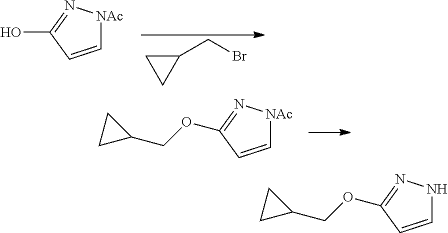

To a suspension of 1-(3-hydroxy-1H-pyrazol-1-yl)ethan-1-one (2.3 g, 18 mmol, 1 eq) and K₂CO₃ (3.79 g, 27.4 mmol, 1.5 eq) in DMF (20 mL) was added (bromomethyl)cyclopropane (2.7 g, 1.1 eq). The reaction mixture was heated at 80° C. (100° C. external) for 4 hours and was stirred at room temperature overnight. EtOAc (100 mL) and H₂O (50 mL) were added and the layers were separated. The aqueous layer was extracted once with EtOAc (100 mL) and the organic layers were combined, washed with H₂O (50 mL), brine, dried over Na₂SO₄ and concentrated. The residue (3.7 g oil) was purified by automated column chromatography (120 g silica) using a gradient of 0-20% EtOAc in heptanes. The product-containing fractions were pooled and concentrated affording 1-(3-(cyclopropylmethoxy)-1H-pyrazol-1-yl)ethan-1-one (2.22 g, 67.6% yield) as an oil which crystallized to a solid. ¹H NMR (DMSO-d₆, 300 MHz): δ 8.21 (d, 1H); 6.19 (d, 1H); 4.01 (d, 2H); 2.47 (s, 3H); 1.28-1.20 (m, 1H); 0.58-0.52 (m, 2H); 0.35-0.30 (m, 2H) ppm

1-(3-(Cyclopropylmethoxy)-1H-pyrazol-1-yl)ethan-1-one (2.22 g, 12.3 mmol) was dissolved in MeOH (40 mL) and 30% NaOH (1.81 g, 13.6 mmol, 1.1 eq), diluted with H₂O (2 mL) was added. The reaction mixture was stirred at room temperature for 4 hours, after which time NMR analysis of a sample showed complete conversion. The solvents were concentrated and EtOAc (25 mL) and H₂O (6 mL) were added. The layers were separated and the organic layer was washed with brine (5 mL), dried over Na₂SO₄ and concentrated to dryness to afford 3-(cyclopropylmethoxy)-1H-pyrazole (1.42 g, 83% yield) as a colorless oil. ¹H NMR (CDCl₃, 300 MHz): δ 7.35 (d, 1H); 5.75 (d, 1H); 3.98 (d, 2H); 1.34-1.24 (m, 1H); 0.65-0.58 (m, 2H); 0.37-0.32 (m, 2H) ppm.

The following table contains a list of pyrazole and cyclic urea reagents that are commercially available:

| | |
|---|---|
| compound | Name |

3-Isopropyl-1H-pyrazole
3-Methoxy-1H-pyrazole
1-Methylimidazolidin-2-one
1-Isopropylimidazolidin-2-one
1-(Cyclopropylmethyl)imidazolidin-2-one
4-Isopropyl-1H-pyrazole

### Example 106: Preparation of (14S,17R)-8-[3-(3,3-dimethylbutoxy)-1H-pyrazol-1-yl]-12,12-dimethyl-17-(pyridin-2-yl)-2λ⁶-thia-3,9,11,18,23-pentaazatetracyclo [17.3.1.111,14.05,10]tetracosa-1(22),5,7,9,19(23),20-hexaene-2,2,4-trione (Compound 320)

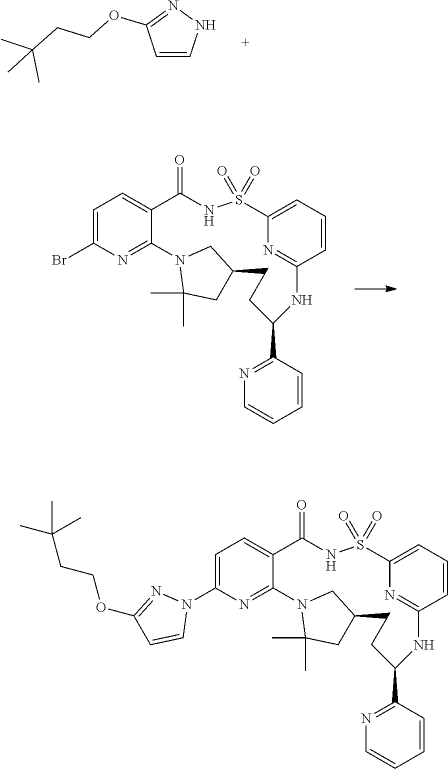 [SMILES: CC(C)(C)CCOc1cc[nH]n1 ; CC1(C)C[C@@H]2CC[C@H](c3ccccn3)Nc3cccc(n3)S(=O)(=O)NC(=O)c3ccc(Br)nc3N1C2 ; CC(C)(C)CCOc1ccn(-c2ccc3c(n2)N2C[C@@H](CC[C@H](c4ccccn4)Nc4cccc(n4)S(=O)(=O)NC3=O)CC2(C)C)n1]

A mixture of (14S,17R)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (25 mg, 0.04375 mmol), 3-(3,3-dimethylbutoxy)-1H-pyrazole (37 mg, 0.2199 mmol), hexaphenyl-1,7-dipalladanonacyclo[5.5.5.01,4.01,11.01,14.02,4.05,7.07, 9.07,16]heptadeca-2,5,8,11,13,16-hexaene-4,10,15-trione (2 mg, 0.0022 mmol), (5-diphenylphosphanyl-9,9-dimethyl-xanthen-4-yl)-diphenyl-phosphane (4 mg, 0.0069 mmol), Cs₂CO₃ (36 mg, 0.110 mmol) and DMF (530 μL) was degassed by bubbling nitrogen for 1 min then heated to 100° C. for 1 h. After cooling to rt more reagents were added: 3-(3,3-dimethylbutoxy)-1H-pyrazole (37 mg, 0.2199 mmol), hexaphenyl-1,7-dipalladanonacyclo [5.5.5.01,4.01,11.01,14.02,4.05,7.07,9.07,16] heptadeca-2,5,8,11,13,16-hexaene-4,10,15-trione (2 mg, 0.0022 mmol), (5-diphenylphosphanyl-9,9-dimethyl-xanthen-4-yl)-diphenyl-phosphane (4 mg, 0.006913 mmol) and Cs₂CO₃ (36 mg, 0.110 mmol), the mixture was degassed by bubbling nitrogen for 1 min then heated to 110° C. for 16 h. Then the mixture was diluted with 2 mL water, 2 mL of 5:1 DCM/MeOH, then 0.6 mL of 1 M HCl added and the mixture partitioned, when paper showed pH 4. The layers were separated and the aqueous layer extracted with 5:1 DCM/MeOH (2 mL). The combined organic extracts were dried (MgSO₄) and evaporated. The residue was dissolved in 1:1 MeOH/ACN at 40 mg/mL and subjected to preparative SFC eluting a gradient of 5 mM NH₃ in methanol to CO₂ (15-50% over 10 min) though a 21.2×250 mm 2-PIC column, 5 μm particle, giving as a white solid the target: (14S,17R)-8-[3-(3,3-dimethylbutoxy)-1H-pyrazol-1-yl]-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (4.5 mg, 16%). ¹H NMR (400 MHz, Chloroform-d) δ 8.54 (d, J=4.8 Hz, 1H), 8.19 (d, J=2.8 Hz, 1H), 8.01 (d, J=8.0 Hz, 1H), 7.71-7.63 (m, 1H), 7.56 (t, J=7.8 Hz, 1H), 7.49 (d, J=7.3 Hz, 1H), 7.32 (d, J=7.8 Hz, 1H), 7.19 (dd, J=7.6, 5.0 Hz, 1H), 6.65 (d, J=8.3 Hz, 1H), 5.90 (d, J=2.8 Hz, 1H), 5.75 (s, 1H), 5.36 (q, J=7.4 Hz, 1H), 4.32 (t, J=7.4 Hz, 2H), 3.48 (s, 1H), 3.02 (t, J=10.4 Hz, 1H), 2.82 (s, 1H), 2.11 (s, 1H), 1.93 (d, J=8.0 Hz, 1H), 1.84 (s, 1H), 1.74 (t, J=7.4 Hz, 2H), 1.64 (s, 4H), 1.62 (s, 3H), 1.59-1.37 (m, 3H), 1.00 (s, 9H). ESI-MS m/z calc. 658.305, found 659.1 (M+1)⁺; Retention time: 1.58 minutes (LC method A).

The compounds in the following table were prepared in a manner analogous to that described above using pyrazole reagents prepared in the examples above and commercially available pyrazole and cyclic urea reagents given in the tables above:

| Compound | | |
|---|---|---|
| Number | Structure | NMR |
| 319 | 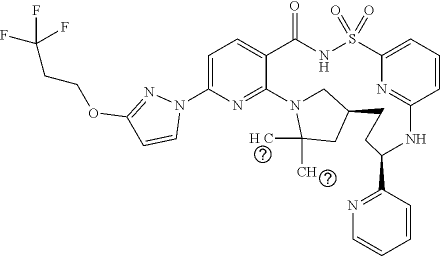 | ¹H NMR (400 MHz, Chloroform-d) δ 8.55 (d, J = 4.8 Hz, 1H), 8.20 (d, J = 2.7 Hz, 1H), 8.02 (d, J = 8.3 Hz, 1H), 7.70- 7.62 (m, 1H), 7.57 (t, J = 7.8 Hz, 1H), 7.50 (d, J = 7.2 Hz, 1H), 7.32 (d, J = 7.8 Hz, 1H), 7.20 (dd, J = 7.6, 4.9 Hz, 2H), 6.66 (d, J = 8.3 Hz, 1H), 5.92 (d, J = 2.7 Hz, 1H), 5.72 (s, 1H), 5.37 (q, J = 7.5 Hz, 1H), 4.52 (t, J = 6.6 Hz, 2H), 3.49 (s, 1H), 3.03 (t, J = 10.3 Hz, 1H), 2.81 (s, 1H), 2.67 (qt, J = 10.6, 6.5 Hz, 2H), 2.12 (t, J = 10.3 Hz, 1H), 1.94 (d, J = 8.0 Hz, 1H), 1.84 (s, 1H), 1.65 (s, 3H), 1.63 (s, 3H), 1.61-1.45 (m, 3H). |
| 321 | 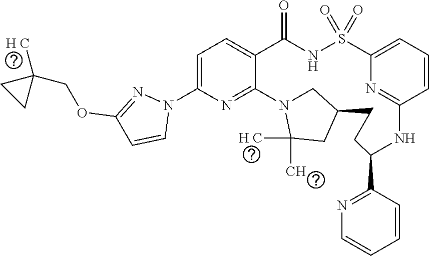 | ¹H NMR (400 MHz, Chloroform-d) δ 8.60-8.52 (m, 1H), 8.20 (d, J = 2.8 Hz, 1H), 8.02 (d, J = 8.4 Hz, 1H), 7.66 (td, J = 7.7, 1.9 Hz, 1H), 7.57 (t, J = 7.8 Hz, 1H), 7.51 (d, J = 7.2 Hz, 1H), 7.30 (d, J = 7.8 Hz, 1H), 7.23-7.17 (m, 2H), 6.66 (d, J = 8.2 Hz, 1H), 5.95 (d, J = 2.7 Hz, 1H), 5.79 (d, J = 8.5 Hz, 1H), 5.36 (q, J = 7.4 Hz, 1H), 4.05 (s, 2H), 3.07-2.96 (m, 1H), 2.87 (s, 1H), 2.23-2.10 (m, 1H), 1.99-1.89 (m, 1H), 1.80 (dt, J = 15.2, 8.4 Hz, 1H), 1.65 (s, 3H), 1.64 (s, 3H), 1.59-1.45 (m, 4H), 1.24 (s, 3H), 0.58 (t, J = 2.9 Hz, 2H), 0.47-0.41 (m, 2H). |
| 322 | 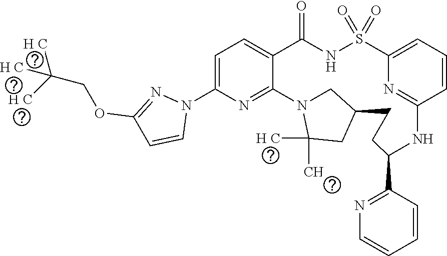 | ¹H NMR (400 MHz, Chloroform-d) δ 8.49 (d, J = 4.8 Hz, 1H), 8.20 (d, J = 2.7 Hz, 1H), 7.99 (d, J = 8.3 Hz, 1H), 7.62 (t, J = 7.9 Hz, 1H), 7.52 (t, J = 7.5 Hz, 1H), 7.44 (d, J = 7.3 Hz, 1H), 7.30 (d, J = 7.8 Hz, 1H), 7.17 (t, J = 6.9 Hz, 2H), 6.63 (d, J = 8.4 Hz, 1H), 5.93 (d, J = 2.8 Hz, 1H), 5.75 (d, J = 8.5 Hz, 1H), 5.37 (d, J = 7.2 Hz, 1H), 3.92 (s, 2H), 3.54-3.48 (m, 1H), 3.02 (t, J = 10.4 Hz, 1H), 2.71 (s, 1H), 2.07 (d, J = 11.5 Hz, 1H), 1.87 (s, 2H), 1.65 (s, 3H), 1.62 (s, 3H), 1.58-1.40 (m, 3H), 1.04 (s, 9H). |
| 323 | 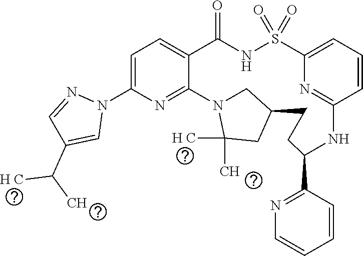 | ¹H NMR (400 MHz, Chloroform-d) δ 8.57 (d, J = 4.8 Hz, 1H), 8.13 (s, 1H), 8.06 (d, J = 8.5 Hz, 1H), 7.66 (t, J = 7.3 Hz, 1H), 7.62 (s, 1H), 7.58 (t, J = 7.9 Hz, 1H), 7.52 (d, J = 7.1 Hz, 1H), 7.39 (d, J = 8.4 Hz, 1H), 7.31 (d, J = 7.7 Hz, 1H), 7.23-7.17 (m, 1H), 6.67 (d, J = 8.3 Hz, 1H), 5.75 (d, J = 8.5 Hz, 1H), 5.47-5.30 (m, 1H), 3.50 (t, J = 8.5 Hz, 1H), 3.04 (t, J = 10.3 Hz, 1H), 2.92 (dt, J = 14.0, 7.0 Hz, 1H), 2.22-2.12 (m, 1H), 1.95 (d, J = 7.7 Hz, 1H), 1.82 (d, J = 7.7 Hz, 1H), 1.68 (s, 3H), 1.59 (d, J = 24.6 Hz, 7H), 1.27 (d, J = 7.2 Hz, 6H) |
| 331 | 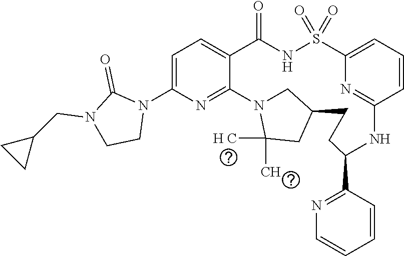 | ¹H NMR (400 MHz, Chloroform-d) δ 8.56 (dd, J = 4.9, 1.7 Hz, 1H), 7.95 (d, J = 8.7 Hz, 1H), 7.82 (d, J = 8.7 Hz, 1H), 7.65 (td, J = 7.7, 1.8 Hz, 1H), 7.54 (d, J = 7.7 Hz, 2H), 7.30 (d, J = 7.8 Hz, 1H), 7.23-7.16 (m, 1H), 6.64 (dd, J = 7.5, 1.7 Hz, 1H), 5.92 (d, J = 8.4 Hz, 1H), 5.34 (q, J = 6.8 Hz, 1H), 4.03 (dd, J = 9.2, 7.0 Hz, 2H), 3.60 (dd, J = 9.3, 7.2 Hz, 2H), 3.51-3.43 (m, 1H), 3.18 (d, J = 7.1 Hz, 2H), 3.09-2.92 (m, 1H), 2.20 (dd, J = 12.4, 8.4 Hz, 1H), 1.93 (dt, J = 14.8, 7.4 Hz, 1H), 1.74 (t, J = 7.8 Hz, 1H), 1.63 (s, 1H), 1.60 (s, 3H), 1.59 (s, 3H), 1.48 (dt, J = 24.7, 9.9 Hz, 3H), 0.95 (tt, J = 7.5, 4.8 Hz, 1H), 0.59-0.48 (m, 2H), 0.25 (q, |
| | | J = 5.1 Hz, 2H). |
| 332 | 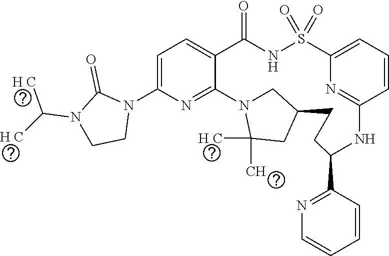 | ¹H NMR (400 MHz, Chloroform-d) δ 8.60-8.54 (m, 1H), 7.96 (d, J = 8.7 Hz, 1H), 7.84 (d, J = 8.6 Hz, 1H), 7.66 (td, J = 7.6, 1.8 Hz, 1H), 7.55 (d, J = 7.0 Hz, 2H), 7.30 (d, J = 7.8 Hz, 1H), 7.20 (ddd, J = 6.2, 5.0, 1.1 Hz, 1H), 6.64 (d, J = 1.3 Hz, 1H), 5.92 (s, 1H), 5.36-5.30 (m, 1H), 4.27 (hept, J = 6.8 Hz, 1H), 4.00 (dd, J = 9.5, 7.0 Hz, 2H), 3.43 (t, J = 8.2 Hz, 2H), 3.07 (s, 1H), 3.01-2.90 (m, 1H), 2.20 (q, J = 8.2, 5.3 Hz, 1H), 1.93 (dt, J = 14.5, 7.5 Hz, 1H), 1.71 (s, 3H), 1.60 (s, 3H), 1.58 (s, 3H), 1.55-1.41 (m, 2H), 1.19 (dd, J = 6.8, 2.7 Hz, 6H). |
| 333 | 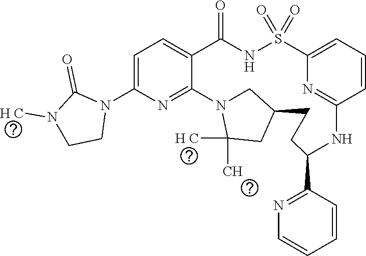 | ¹H NMR (400 MHz, Chloroform-d) δ 8.59-8.55 (m, 1H), 7.96 (d, J = 8.7 Hz, 1H), 7.82 (d, J = 8.7 Hz, 1H), 7.65 (td, J = 7.7, 1.8 Hz, 1H), 7.58-7.49 (m, 2H), 7.30 (d, J = 7.8 Hz, 1H), 7.20 (ddd, J = 7.6, 4.8, 1.1 Hz, 1H), 6.64 (dd, J = 7.4, 1.6 Hz, 1H), 5.90 (d, J = 8.3 Hz, 1H), 5.34 (q, J = 6.9 Hz, 1H), 4.00 (dd, J = 9.1, 7.0 Hz, 2H), 3.51-3.44 (m, 3H), 3.11- 2.93 (m, 1H), 2.90 (s, 3H), 2.20 (t, J = 10.5 Hz, 1H), 1.93 (dt, J = 14.6, 7.5 Hz, 1H), 1.72 (q, J = 14.0, 11.1 Hz, 1H), 1.63 (s, 1H), 1.60 (s, 3H), 1.58 (s, 3H), 1.57- 1.41 (m, 3H). |
| 334 | 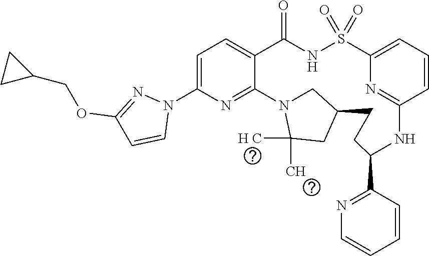 | ¹H NMR (400 MHz, Chloroform-d) δ 8.55 (d, J = 4.9 Hz, 1H), 8.19 (d, J = 2.8 Hz, 1H), 8.01 (d, J = 8.3 Hz, 1H), 7.65 (td, J = 7.7, 1.8 Hz, 1H), 7.56 (t, J = 7.8 Hz, 1H), 7.50 (d, J = 7.3 Hz, 1H), 7.31 (d, J = 7.7 Hz, 1H), 7.24-7.20 (m, 1H), 7.20-7.17 (m, 1H), 6.66 (d, J = 8.3 Hz, 1H), 5.94 (d, J = 2.7 Hz, 1H), 5.78 (d, J = 8.5 Hz, 1H), 5.36 (q, J = 7.4 Hz, 1H), 4.10 (d, J = 7.2 Hz, 2H), 3.48 (s, 1H), 3.02 (t, J = 10.3 Hz, 1H), 2.87 (d, J = 10.5 Hz, 1H), 2.14 (t, J = 10.2 Hz, 1H), 2.00-1.89 (m, 1H), 1.86-1.75 (m, 1H), 1.65 (s, 3H), 1.63 (s, 3H), 1.61-1.44 (m, 4H), 1.37-1.27 (m, 1H), 0.68-0.60 (m, 2H), 0.39 (dt, J = 6.2, 4.6 Hz, 2H). |
| 335 | 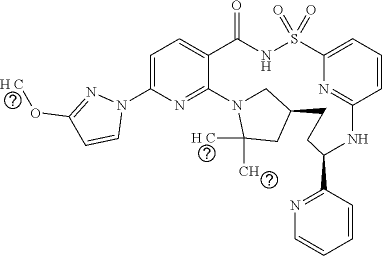 | ¹H NMR (400 MHz, Chloroform-d) δ 8.54 (d, J = 4.9 Hz, 1H), 8.20 (d, J = 2.7 Hz, 1H), 8.02 (d, J = 8.3 Hz, 1H), 7.66 (td, J = 7.7, 1.8 Hz, 1H), 7.56 (t, J = 7.8 Hz, 1H), 7.50 (d, J = 7.3 Hz, 1H), 7.31 (d, J = 7.8 Hz, 1H), 7.24 (d, J = 8.8 Hz, 1H), 7.19 (ddd, J = 7.6, 4.8, 1.1 Hz, 1H), 6.66 (d, J = 8.3 Hz, 1H), 5.92 (d, J = 2.7 Hz, 1H), 5.76 (d, J = 8.5 Hz, 1H), 5.37 (q, J = 7.6 Hz, 1H), 3.98 (s, 3H), 3.52- 3.46 (m, 1H), 3.02 (t, J = 10.3 Hz, 1H), 2.86 (d, J = 15.3 Hz, 1H), 2.19-2.09 (m, 1H), 2.00-1.90 (m, 1H), 1.82 (d, J = 8.4 Hz, 1H), 1.65 (s, 3H), 1.63 (s, 3H), 1.54 (dt, J = 33.2, 11.5 Hz, 3H). |
| 336 | 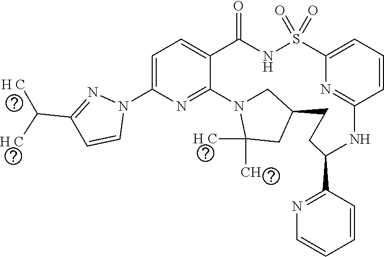 | ¹H NMR (400 MHz, Chloroform-d) δ 8.57-8.48 (m, 1H), 8.29 (d, J = 2.7 Hz, 1H), 8.02 (d, J = 8.3 Hz, 1H), 7.64 (td, J = 7.7, 1.8 Hz, 1H), 7.58-7.52 (m, 1H), 7.48 (d, J = 7.2 Hz, 1H), 7.36 (d, J = 8.3 Hz, 1H), 7.30 (d, J = 7.8 Hz, 1H), 7.18 (ddd, J = 7.6, 4.8, 1.1 Hz, 1H), 6.65 (d, J = 8.2 Hz, 1H), 6.29 (d, J = 2.6 Hz, 1H), 5.75 (d, J = 8.6 Hz, 1H), 5.38 (td, J = 8.4, 5.4 Hz, 1H), 3.53-3.44 (m, 1H), 3.16- 2.98 (m, 2H), 2.79 (d, J = 8.7 Hz, 1H), 2.11 (dd, J = 12.2, 7.6 Hz, 1H), 1.99- 1.79 (m, 1H), 1.67 (s, 1H), 1.66 (s, 3H), 1.64 (s, 3H), 1.63-1.46 (m, 3H), 1.34- 1.28 (m, 6H). |

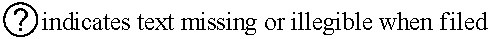

| Compound | LCMS Retention | Exact | | |
|---|---|---|---|---|
| Number | Time (min) | Mass | M + 1 | LCMS Method |
| 319 | 1.34 | 670.23 | 671.1 | LC method A |
| 321 | 1.47 | 642.274 | 643.5 | LC method A |
| 322 | 1.52 | 644.289 | 645.2 | LC method A |
| 323 | 1.35 | 600.263 | 601.4 | LC method A |
| 331 | 1.13 | 630.274 | 631.5 | LC method A |
| 332 | 1.07 | 618.274 | 619.5 | LC method A |
| 333 | 0.88 | 590.242 | 591.5 | LC method A |
| 334 | 1.36 | 628.258 | 629.5 | LC method A |
| 335 | 1.16 | 588.227 | 589.5 | LC method A |
| 336 | 1.37 | 600.263 | 601.5 | LC method A |

### Example 107: Preparation of (14S,17R)-8-(4,4-Difluorocyclohexyl)-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione

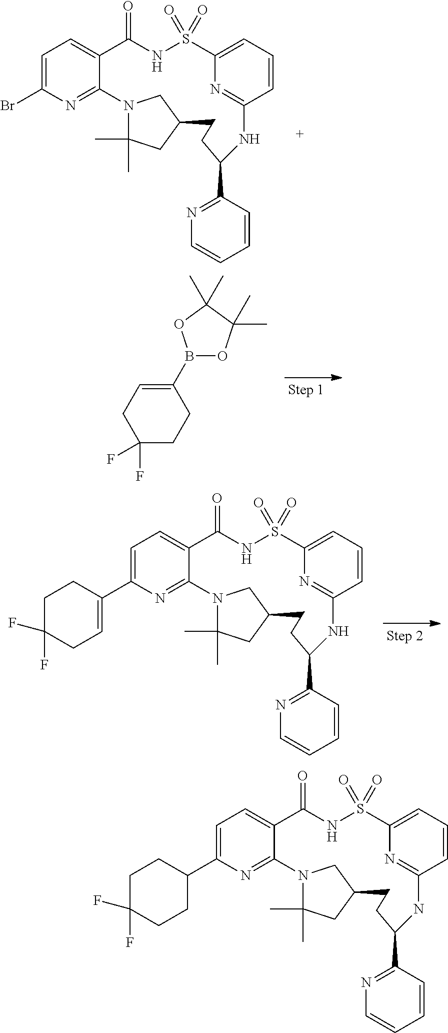

### Step 1: (14S,17R)-8-(4,4-Difluorocyclohex-1-en-1-yl)-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (Compound 325)

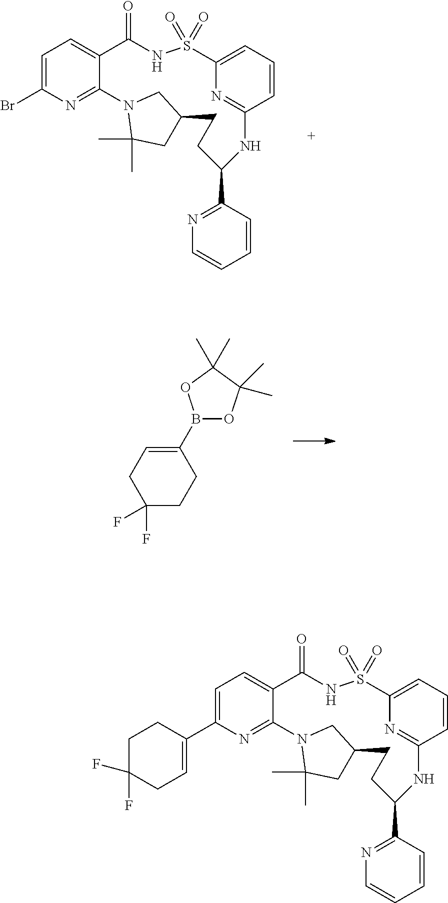 [SMILES: CC1(C)C[C@@H]2CC[C@H](c3ccccn3)Nc3cccc(n3)S(=O)(=O)NC(=O)c3ccc(Br)nc3N1C2 ; CC1(C)OB(C2=CCC(F)(F)CC2)OC1(C)C ; CC1(C)C[C@@H]2CC[C@H](c3ccccn3)Nc3cccc(n3)S(=O)(=O)NC(=O)c3ccc(C4=CCC(F)(F)CC4)nc3N1C2]

A mixture of (14S,17R)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (25 mg, 0.0437 mmol), 2-(4,4-difluorocyclohexen-1-yl)-4,4,5,5-tetramethyl-1,3,2-dioxaborolane (43 mg, 0.176 mmol), Pd(dppf)Cl₂ (3.3 mg, 0.0045 mmol), DMF (437 μL) and aqueous sodium bicarbonate (260 μL of 1 M, 0.2600 mmol) was degassed by bubbling nitrogen for 1 min then heated to 100° C. for 1 h. Then the mixture was diluted with 2 mL water, 2 mL of 5:1 DCM/MeOH, then 0.6 mL of 1 M HCl added and the mixture partitioned, when paper showed pH 4. The layers were separated, and the aqueous layer extracted with 5:1 DCM/MeOH (2 mL). The residue was dissolved in 1:1 MeOH/ACN at 40 mg/mL and subjected to preparative SFC eluting a gradient of 5 mM NH₃ in methanol to CO₂ (15-50% over 10 min) through a 21.2×250 mm 2-PIC column, 5 μm particle, giving as a white solid the target: (14S,17R)-8-(4,4-difluorocyclohex-1-en-1-yl)-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (23 mg, 86%). ¹H NMR (400 MHz, Chloroform-d) δ 8.57 (d, J=4.8 Hz, 1H), 7.98 (d, J=8.1 Hz, 1H), 7.66 (td, J=7.7, 1.8 Hz, 1H), 7.60-7.50 (m, 2H), 7.29 (d, J=7.7 Hz, 1H), 7.23-7.16 (m, 1H), 6.98 (d, J=8.1 Hz, 1H), 6.69-6.65 (m, 1H), 6.56 (s, 1H), 5.94 (d, J=8.3 Hz, 1H), 5.35 (q, J=7.1 Hz, 1H), 3.51-3.40 (m, 1H), 3.14-3.03 (m, 1H), 3.00 (d, J=9.5 Hz, 1H), 2.83-2.69 (m, 4H), 2.19 (ddt, J=20.4, 13.6, 7.5 Hz, 3H), 1.94 (dt, J=14.7, 7.5 Hz, 1H), 1.71 (dd, J=14.2, 7.3 Hz, 1H), 1.63 (s, 6H), 1.59-1.42 (m, 3H). ESI-MS m/z calc. 608.2381, found 609.1 (M+1)⁺; Retention time: 1.26 minutes (LC method A).

### Step 2: (14S,17R)-8-(4,4-Difluorocyclohexyl)-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (Compound 324)

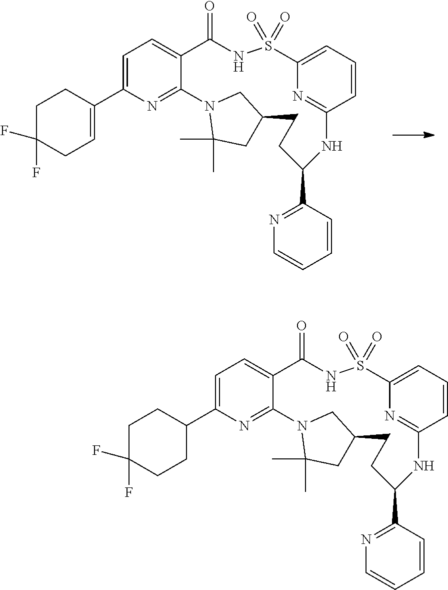 [SMILES: CC1(C)C[C@@H]2CC[C@H](c3ccccn3)Nc3cccc(n3)S(=O)(=O)NC(=O)c3ccc(C4=CCC(F)(F)CC4)nc3N1C2 ; CC1(C)C[C@@H]2CC[C@H](c3ccccn3)Nc3cccc(n3)S(=O)(=O)NC(=O)c3ccc(C4CCC(F)(F)CC4)nc3N1C2]

A mixture of (14S,17R)-8-(4,4-difluorocyclohex-1-en-1-yl)-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (20.00 mg, 0.0329 mmol), Pd(OH)₂ (11.54 mg of 20% w/w, 0.0164 mmol) and EtOAc (1 mL) was stirred under balloon pressure hydrogen for 16 h. Then the mixture was filtered, concentrated and the residue dissolved in EtOH (1 mL) and PtO₂ (3.731 mg, 0.0164 mmol) was added and the mixture was subjected to Parr shaker hydrogenation at 55 psi hydrogen for 2 h, filtered over Celite, solvent evaporated and the residue was dissolved in MeOH and filtered through a 0.45 μm filter and the filtrate subjected to preparative SFC eluting a gradient of 5 mM NH₃ in methanol to CO₂ (15-50% over 10 min) though a 21.2×250 mm 2-PIC column, 5 μm particle, giving as a white solid the target: (14S,17R)-8-(4,4-difluorocyclohexyl)-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (1.7 mg, 8%). ¹H NMR (400 MHz, Chloroform-d) δ 8.60-8.52 (m, 1H), 7.93 (d, J=7.8 Hz, 1H), 7.66 (td, J=7.7, 1.8 Hz, 1H), 7.57 (t, J=7.7 Hz, 1H), 7.53 (d, J=7.2 Hz, 1H), 7.29 (d, J=7.9 Hz, 1H), 7.21 (dd, J=7.5, 5.0 Hz, 1H), 6.68 (dd, J=10.8, 8.0 Hz, 2H), 5.89 (d, J=8.3 Hz, 1H), 5.34 (q, J=7.1 Hz, 1H), 3.48-3.39 (m, 1H), 3.04-2.92 (m, 2H), 2.68 (s, 1H), 2.25-2.14 (m, 3H), 1.99-1.87 (m, 6H), 1.85-1.65 (m, 2H), 1.63 (s, 3H), 1.61 (s, 3H), 1.59-1.36 (m, 3H). ESI-MS m/z calc. 610.2538, found 611.4 (M+1)⁺; Retention time: 1.31 minutes (LC method S).

### Example 108: Preparation of (14S,17R)-8-cyclobutyl-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (Compound 330)

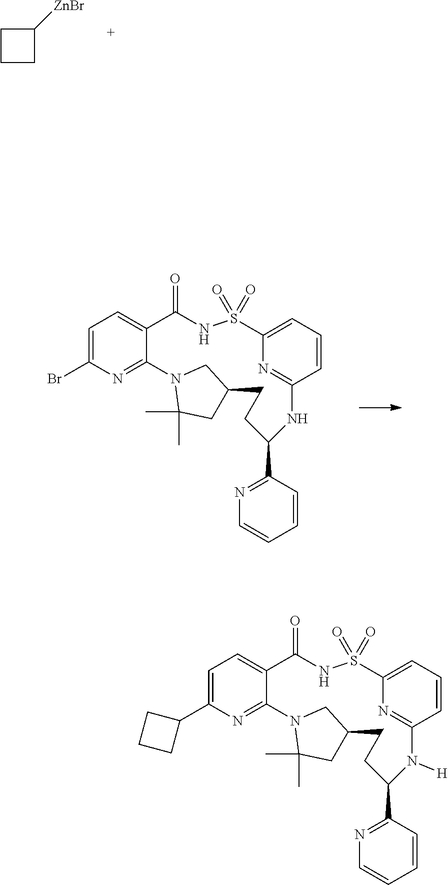

A mixture of (14S)-8-bromo-12,12-dimethyl-17-(pyridin-2-yl)-2λ⁶-thia-3,9,11,18,23-pentaazatetracyclo[17.3.1.111,14.05,10]tetracosa-1(22),5(10),6,8,19(23),20-hexaene-2,2,4-trione (30 mg, 0.05249 mmol), bromo(cyclobutyl)zinc (315 μL of 0.5 M in THF, 0.1575 mmol), and Pd(PPh₃)₄ (6 mg, 0.005192 mmol) in DMF (520 μL) was bubbled with nitrogen for 1 min and then heated at 100° C. for 3 h then more reagents were added: bromo(cyclobutyl)zinc (630 μL of 0.5 M, 0.3150 mmol) and Pd(PPh₃)₄ (6 mg, 0.005192 mmol) and the mixture heated to 100° C. for 3 h. Then the mixture was diluted with 2 mL water, 2 mL of 5:1 DCM/MeOH, then 0.6 mL of 1 M HCl added and the mixture partitioned, when paper showed pH 3. The layers were separated, and the aqueous layer extracted with 5:1 DCM/MeOH (2 mL). The combined organic extracts were dried (MgSO₄) and evaporated. The residue was dissolved in 1:1 MeOH/ACN and subjected to preparative SFC eluting a gradient of 5 mM NH₃ in methanol to CO₂ (15-50% over 10 min) though a 21.2×250 mm 2-PIC column, 5 μm particle, with a series of 400 μL injections giving as a white solid (after evaporation of the collected fractions) the target: (14S,17R)-8-cyclobutyl-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (15.8 mg, 55%). ¹H NMR (400 MHz, Chloroform-d) δ 8.57 (d, J=4.8 Hz, 1H), 7.91 (d, J=7.8 Hz, 1H), 7.66 (td, J=7.7, 1.8 Hz, 1H), 7.60-7.52 (m, 2H), 7.29 (d, J 7.8 Hz, 1H), 7.21 (dd, J=7.6, 4.9 Hz, 1H), 6.70-6.61 (m, 2H), 5.96 (d, J=8.2 Hz, 1H), 5.35 (q, J=6.8 Hz, 1H), 3.56 (p, J=8.6 Hz, 1H), 3.46 (dd, J=9.7, 6.8 Hz, 1H), 3.10 (d, J=9.5 Hz, 1H), 3.00 (t, J 10.0 Hz, 1H), 2.40-2.21 (m, 5H), 2.10-1.98 (m, 1H), 1.97-1.85 (m, 2H), 1.77-1.69 (m, 1H), 1.68 (s, 3H), 1.67 (s, 3H), 1.64-1.43 (m, 3H). ESI-MS m/z calc. 546.24133, found 547.4 (M+1)⁺; Retention time: 1.29 minutes (LC method A).

### Example 109: Preparation of (14S,17R)-8-(6-fluoropyridin-2-yl)-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (Compound 318)

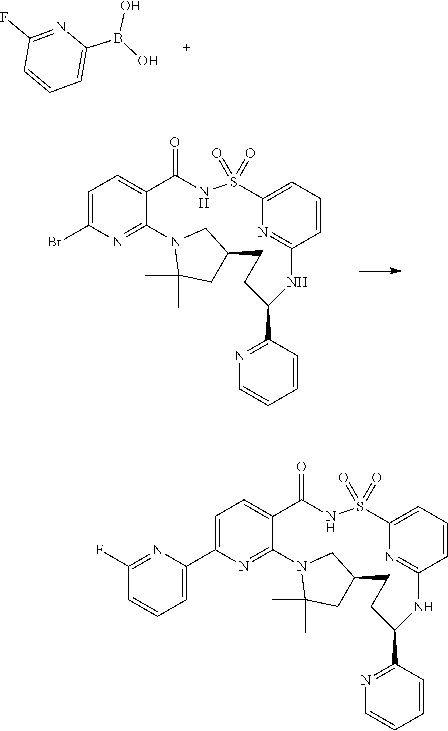 [SMILES: OB(O)c1cccc(F)n1 ; CC1(C)C[C@@H]2CC[C@H](c3ccccn3)Nc3cccc(n3)S(=O)(=O)NC(=O)c3ccc(Br)nc3N1C2 ; CC1(C)C[C@@H]2CC[C@H](c3ccccn3)Nc3cccc(n3)S(=O)(=O)NC(=O)c3ccc(-c4cccc(F)n4)nc3N1C2]

A mixture of (14S,17R)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (250 mg, 0.4375 mmol), (6-fluoro-2-pyridyl)boronic acid (185 mg, 1.313 mmol), Pd(dppf)Cl₂ (32 mg, 0.044 mmol), acetonitrile (4.4 mL) and NaHCO₃(2.6 mL of 1 M, 2.600 mmol) was degassed by bubbling nitrogen for 1 min then heated to 100° C. for 3 h. Then the mixture was diluted with 2 mL water, 2 mL of 5:1 DCM/MeOH, then 0.5 mL of 1 M HCl added and the mixture partitioned, when paper showed pH 4. The layers were separated and the aqueous layer extracted with 5:1 DCM/MeOH (2 mL). The residue was purified by flash chromatography (12 g SiO₂, 50-80% EtOAc in hexanes over 15 min) to provide the target: (14S,17R)-8-(6-fluoropyridin-2-yl)-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (180 mg, 70%). ¹H NMR (400 MHz, Chloroform-d) δ 8.56 (d, J=4.8 Hz, 1H), 8.17 (dd, J=7.5, 2.3 Hz, 1H), 8.09 (d, J=8.0 Hz, 1H), 7.92 (t, J=8.0 Hz, 1H), 7.86 (d, J=8.2 Hz, 1H), 7.66 (td, J=7.6, 1.8 Hz, 1H), 7.58 (t, J=7.7 Hz, 1H), 7.53 (d, J=7.2 Hz, 1H), 7.31 (d, J=7.8 Hz, 1H), 7.20 (dd, J=7.5, 4.9 Hz, 1H), 6.96 (dd, J=8.2, 2.9 Hz, 1H), 6.67 (d, J=8.2 Hz, 1H), 5.85 (d, J=8.5 Hz, 1H), 5.38 (q, J=7.4 Hz, 1H), 3.51 (dd, J=9.7, 6.8 Hz, 1H), 3.04 (t, J=10.2 Hz, 1H), 2.94 (d, J=9.5 Hz, 1H), 2.20 (dd, J=12.3, 8.0 Hz, 1H), 1.95 (q, J=7.1 Hz, 1H), 1.80 (dt, J=15.0, 8.2 Hz, 1H), 1.72 (s, 3H), 1.71 (s, 3H), 1.68-1.58 (m, 2H), 1.52 (q, J=12.1, 10.8 Hz, 1H). ESI-MS m/z calc. 587.2115, found 588.1 (M+1)⁺; Retention time: 1.14 minutes (LC method A).

The following is a list of boronic acid reagents that are commercially available:
(4-Isopropylphenyl)boronic acid
1-Isopropylpyrazol-4-yl)boronic acid
(2-Chloro-6-fluoro-phenyl)boronic acid
(2,6-Dichlorophenyl)boronic acid
(3-Methoxyphenyl)boronic acid
(6-Methoxy-2-pyridyl)boronic acid
Cyclohexen-1-ylboronic acid
2-(4,4-Dimethylcyclohexen-1-yl)-4,4,5,5-tetramethyl-1,3,2-dioxaborolane
Cyclopropylboronic acid

The compounds in the following table were prepared in a manner analogous to that described above using boronic acids given in the tables above:

| Compound | | |
|---|---|---|
| Number | Structure | NMR |
| 337 | 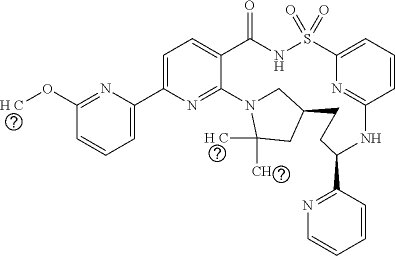 | ¹H NMR (400 MHz, Chloroform-d) δ 8.57 (dt, J = 4.8, 1.2 Hz, 1H), 8.13 (d, J = 8.0 Hz, 1H), 7.97 (d, J = 8.0 Hz, 1H), 7.92-7.86 (m, 1H), 7.70 (d, J = 7.9 Hz, 1H), 7.69-7.63 (m, 1H), 7.60-7.53 (m, 2H), 7.31 (dd, J =7.9, 1.3 Hz, 1H), 7.21 (ddd, J = 7.6, 4.9, 1.1 Hz, 1H), 6.82- 6.77 (m, 1H), 6.67 (dd, J = 7.5, 1.6 Hz, 1H), 5.95 (d, J = 8.3 Hz, 1H), 5.41-5.33 (m, 1H), 4.04 (s, 3H), 3.55-3.49 (m, 1H), 3.04 (dd, J = 12.7, 6.3 Hz, 2H), 2.26 (dd, J = 12.4, 8.2 Hz, 1H), 1.95 (dt, J = 14.6, 7.3 Hz, 1H), 1.77 (d, J = 8.3 Hz, 1H), 1.74 (s, 3H), 1.71 (s, 3H), 1.67- 1.46 (m, 3H). |
| 339 | 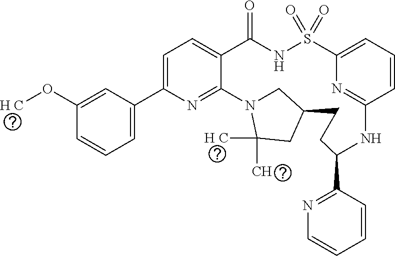 | ¹H NMR (400 MHz, Chloroform-d) δ 8.55 (d, J = 4.9 Hz, 1H), 8.04 (d, J = 8.0 Hz, 1H), 7.72-7.61 (m, 2H), 7.58 (d, J = 7.5 Hz, 1H), 7.55 (d, J = 3.3 Hz, 2H), 7.37 (t, J = 7.9 Hz, 1H), 7.29 (t, J = 9.1 Hz, 2H), 7.20 (dd, J = 7.6, 4.9 Hz, 1H), 6.98 (dd, J = 8.1, 2.6 Hz, 1H), 6.67 (d, J = 7.8 Hz, 1H), 5.92 (d, J = 8.3 Hz, 1H), 5.38 (q, J = 7.1 Hz, 1H), 3.87 (s, 3H), 3.52 (s, 1H), 3.04 (s, 1H), 2.22 (t, J = 9.5 Hz, 1H), 1.95 (dt, J = 14.2, 7.2 Hz, 1H), 1.73 (s, 3H), 1.71 (s, 3H), 1.68- 1.53 (m, 5H). |
| 341 | 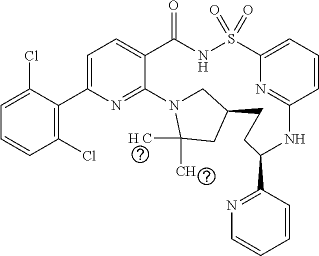 | ¹H NMR (400 MHz, DMSO-d₆) δ 12.4 (br s, 1H), 8.52 (d, J = 4.3 Hz, 1H), 7.81 (d, J = 7.7 Hz, 1H), 7.75 (dd, J = 7.9, 1.8 Hz, 1H), 7.73-7.68 (m, 1H), 7.65 (t, J = 7.9 Hz, 1H), 7.59 (d, J = 8.0 Hz, 1H), 7.55 (d, J = 7.9 Hz, 1H), 7.47 (d, J = 2.5 Hz, 1H), 7.46-7.42 (m, 1H), 7.24 (dd, J = 7.5, 4.8 Hz, 1H), 7.12 (d, J = 7.2 Hz, 1H), 6.89 (d, J = 8.5 Hz, 1H), 6.61 (d, J = 7.7 Hz, 1H), 5.29 (s, 1H), 2.79 (t, J = 10.6 Hz, 1H), 2.3-2.2 (m, 2H), 1.95 (d, J = 9.0 Hz, 2H), 1.83 (dd, J = 11.8, 5.1 Hz, 1H), 1.74 (d, J = 13.9 Hz, 1H), 1.60 (d, J = 12.1 Hz, 1H), 1.52 (s, 3H), 1.47 (s, 1H), 1.41 (s, 3H). |
| 343 | 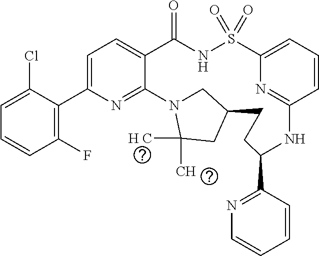 | ¹H NMR (400 MHz, DMSO-d₆) δ 8.52 (s, 1H),7.73 (td, J = 25.7, 23.3,10.4 Hz, 3H), 7.54-7.29 (m, 4H), 7.23 (s, 1H), 7.12 (d, J = 7.0 Hz, 1H), 6.90 (d, J = 8.9 Hz, 1H), 6.70 (d, J = 7.8 Hz, 1H), 5.39 (s, 1H), 5.29 (s, 1H), 2.79 (s, 1H), 2.23 (s, 1H), 1.95 (d, J = 11.6 Hz, 2H), 1.79 (d, J = 28.8 Hz, 2H), 1.60 (d, J = 11.7 Hz, 1H), 1.53 (s, 4H), 1.41 (s, 4H). |
| 346 | 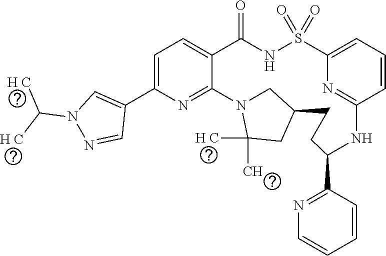 | ¹H NMR (400 MHz, Chloroform-d) δ 8.62-8.55 (m, 1H), 7.99 (d, J = 8.0 Hz, 2H), 7.87 (s, 1H), 7.66 (td, J = 7.7, 1.8 Hz, 1H), 7.61-7.51 (m, 2H), 7.30 (d, J = 7.8 Hz, 1H), 7.24-7.18 (m, 1H), 7.00 (d, J = 8.0 Hz, 1H), 6.66 (dd, J = 7.2, 1.9 Hz, 1H), 5.97 (d, J = 8.3 Hz, 1H), 5.35 (q, J = 6.9 Hz, 1H), 4.54 (hept, J = 6.6 Hz, 1H), 3.50 (dd, J = 9.6, 6.6 Hz, 1H), 3.10 (s, 1H), 3.01 (t, J = 10.0 Hz, 1H), 2.26 (dd, J = 12.4, 8.8 Hz, 1H), 1.94 (dt, J = 14.5, 7.6 Hz, 1H), 1.74 (d, J = 10.0 Hz, 1H), 1.70 (s, 3H), 1.67 (s, 3H), 1.65-1.57 (m,2H), 1.56 (d, J = 6.7 Hz, 6H), 1.53-1.43 (m, 1H). |
| 347 | 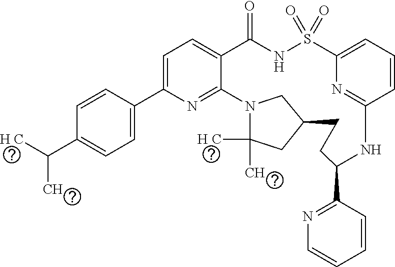 | ¹H NMR (400 MHz, Chloroform-d) δ 8.61-8.50 (m, 1H), 8.06 (d, J = 8.1 Hz, 1H), 7.95 (d, J = 8.3 Hz, 2H), 7.67 (td, J = 7.7, 1.8 Hz, 1H), 7.61-7.50 (m, 2H), 7.35-7.28 (m, 4H), 7.22 (dd, J = 7.6, 4.8 Hz, 1H), 6.67 (dd, J = 7.4, 1.8 Hz, 1H), 5.99 (d, J = 8.3 Hz, 1H), 5.37 (q, J = 6.9 Hz, 1H), 3.56-3.48 (m, 1H), 3.17- 2.91 (m, 2H), 2.26 (dd, J = 12.4, 8.3 Hz, 1H), 1.94 (dt, J = 14.4, 7.4 Hz, 1H), 1.78 (s, 1H), 1.73 (s, 3H), 1.71 (s, 3H), 1.67-1.46 (m, 3H), 1.29 (d, J = 6.9 Hz, 6H), 1.27-1.20 (m, 1H). |
| 326 | 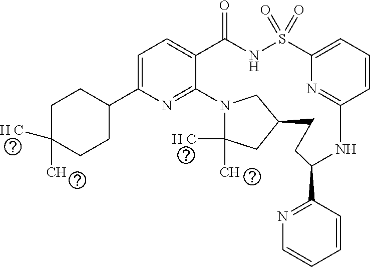 | ¹H NMR (400 MHz, Chloroform-d) δ 8.61-8.55 (m, 1H), 7.90 (s, 1H), 7.66 (t, J = 7.4 Hz, 1H), 7.55 (q, J = 7.2 Hz, 2H), 7.30 (d, J = 7.9 Hz, 1H), 7.20 (dd, J = 7.6, 5.0 Hz, 1H), 6.64 (d, J = 8.5 Hz, 2H), 5.92 (s, 1H), 5.34 (q, J = 7.0 Hz, 1H), 3.46 (s, 1H), 2.99 (t, J = 9.8 Hz, 1H), 2.47 (d, J = 12.2 Hz, 1H), 2.20 (s, 1H), 1.98-1.84 (m, 1H), 1.81-1.66 (m, 5H), 1.63 (s, 6H), 1.60-1.23 (m, 8H), 0.96 (s, 3H), 0.95 (s, 3H). |
| 328 | 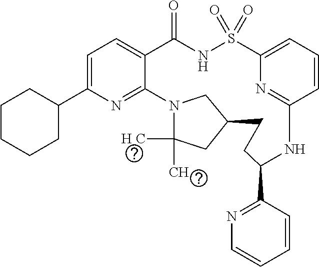 | ¹H NMR (400 MHz, Chloroform-d) δ 8.56 (d, J = 4.8 Hz, 1H), 7.90 (s, 1H), 7.66 (t, J = 7.5 Hz, 1H), 7.54 (d, J = 9.5 Hz, 2H), 7.29 (d, J = 7.8 Hz, 1H), 7.20 (t, J = 6.3 Hz, 1H), 6.64 (d, J = 7.8 Hz, 2H), 5.90 (s, 1H), 5.34 (d, J = 7.5 Hz, 1H), 3.46 (s, 1H), 2.99 (s, 2H), 2.55 (d, J = 12.2 Hz, 1H),2.17 (s, 1H), 1.85 (t, J = 14.9 Hz, 6H), 1.74 (d, J = 11.8 Hz, 2H), 1.62 (d, J = 3.6 Hz, 6H), 1.55-1.17 (m, 7H). |
| 327 | 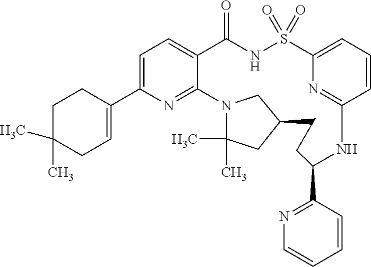 [SMILES: CC1(C)CC=C(c2ccc3c(n2)N2C[C@@H](CC[C@H](c4ccccn4)Nc4cccc(n4)S(=O)(=O)NC3=O)CC2(C)C)CC1] | ¹H NMR (400 MHz, Chloroform-d) δ 8.60-8.56 (m, 1H), 7.99 (d, J = 8.1 Hz, 1H), 7.67 (td, J = 7.7, 1.8 Hz, 1H), 7.60- 7.53 (m, 2H), 7.29 (d, J = 7.8 Hz, 1H), 7.23-7.18 (m, 1H), 7.01 (d, J = 8.1 Hz, 1H), 6.77-6.72 (m, 1H), 6.70-6.59 (m, 1H), 6.00 (d, J = 8.1 Hz, 1H), 5.34 (q, J = 7.0 Hz, 1H), 3.53-3.42 (m, 1H), 3.17 (s, 1H), 2.99 (t, J = 10.2 Hz, 1H), 2.48 (s, 2H), 2.27 (dd, J = 12.4, 9.4 Hz, 1H), 2.06 (dd, J = 4.5, 2.4 Hz, 2H), 1.93 (dt, J = 14.4, 7.6 Hz, 1H), 1.69 (d, J = 15.0 Hz, 1H), 1.65 (s, 3H), 1.64 (s, 3H), 1.60- 1.40 (m, 4H), 0.96 (d, J = 1.7 Hz, 7H). |
| 329 | 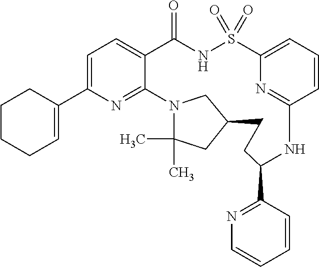 [SMILES: CC1(C)C[C@@H]2CC[C@H](c3ccccn3)Nc3cccc(n3)S(=O)(=O)NC(=O)c3ccc(C4=CCCCC4)nc3N1C2] | ¹H NMR (400 MHz, Chloroform-d) δ 8.60-8.55 (m, 1H), 7.98 (d, J = 8.1 Hz, 1H), 7.67 (td, J = 7.7, 1.8 Hz, 1H), 7.60- 7.53 (m, 2H), 7.29 (d, J = 7.8 Hz, 1H), 7.21 (ddd, J = 7.6, 4.9, 1.1 Hz, 1H), 6.99 (d, J = 8.1 Hz, 1H), 6.84-6.79 (m, 1H), 6.70-6.63 (m, 1H), 6.00 (d, J = 8.2 Hz, 1H), 5.34 (q, J = 7.0 Hz, 1H), 3.47 (dd, J = 9.7, 6.9 Hz, 1H), 3.17 (d, J = 9.7 Hz, 1H), 2.98 (t, J = 10.2 Hz, 1H), 2.54- 2.41 (m, 2H), 2.32-2.22 (m, 4H), 1.93 (dt, J = 14.2, 7.6 Hz, 1H), 1.78 (p, J = 6.1 Hz, 3H), 1.69 (q, J = 6.6, 5.9 Hz, 1H), 1.65 (s, 3H), 1.64 (s, 3H), 1.60- 1.40 (m, 3H). |
| 349 | 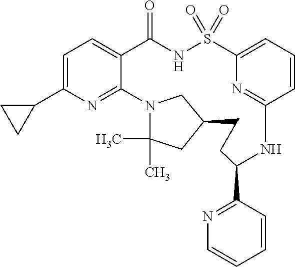 [SMILES: CC1(C)C[C@@H]2CC[C@H](c3ccccn3)Nc3cccc(n3)S(=O)(=O)NC(=O)c3ccc(C4CC4)nc3N1C2] | ¹H NMR (400 MHz, Chloroform-d) δ 10.10 (s, 1H), 8.58 (ddd, J = 4.9, 1.9, 0.9 Hz, 1H), 7.89 (d, J = 7.9 Hz, 1H), 7.66 (td, J = 7.7, 1.8 Hz, 1H), 7.61-7.50 (m, 2H), 7.32-7.26 (m, 1H), 7.21 (ddd, J = 7.6, 4.9, 1.2 Hz, 1H), 6.80 (d, J = 7.9 Hz, 1H), 6.69-6.56 (m, 1H), 5.99 (d, J = 8.2 Hz, 1H), 5.33 (q, J = 7.0 Hz, 1H), 3.43 (dd, J = 9.8, 6.9 Hz, 1H), 3.13 (d, J = 10.4 Hz, 1H), 2.95 (t, J = 10.2 Hz, 1H), 2.24 (dd, J = 12.5, 9.3 Hz, 1H), 2.01-1.86 (m, 2H), 1.74-1.59 (m, 2H), 1.56 (s, 3H), 1.55 (s, 3H), 1.48 (ddd, J = 22.5, 13.2, 6.9 Hz, 2H), 1.05 (ddp, J = 7.2, 4.1, 2.4 Hz, 2H), 1.01-0.94 (m, 2H). |

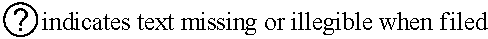

| Compound | LCMS Retention | Exact | | |
|---|---|---|---|---|
| Number | Time (min) | Mass | M + 1 | LCMS Method |
| 337 | 1.11 | 599.231 | 600.4 | LC method A |
| 339 | 1.29 | 598.236 | 599.4 | LC method A |
| 341 | 1.39 | 636.148 | 637.4 | LC method A |
| 343 | 1.3 | 620.177 | 621.1 | LC method A |
| 346 | 1.08 | 600.263 | 601.1 | LC method A |
| 347 | 1.53 | 610.273 | 611.1 | LC method A |
| 326 | 1.63 | 602.304 | 603.5 | LC method A |
| 328 | 1.44 | 574.273 | 575.3 | LC method A |
| 327 | 1.59 | 600.288 | 601.5 | LC method A |
| 329 | 1.42 | 572.257 | 573.5 | LC method A |
| 349 | 1.17 | 532.226 | 533 | LC method A |

### Example 110: Preparation of (14S,17R)-8-[6-(cyclobutylmethoxy)pyridin-2-yl]-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (Compound 313)

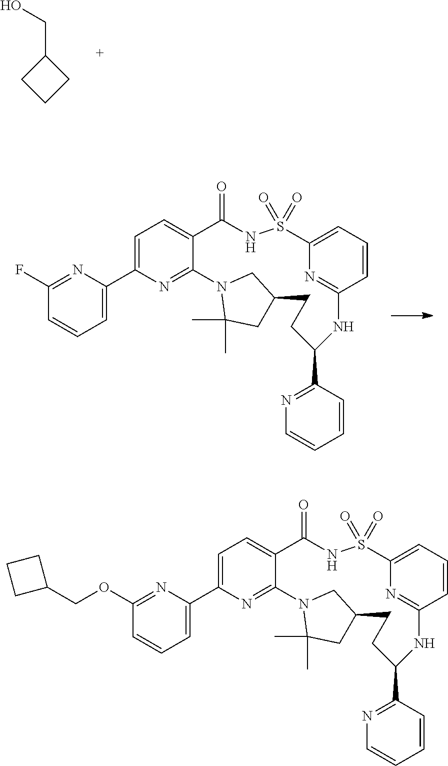 [SMILES: OCC1CCC1 ; CC1(C)C[C@@H]2CC[C@H](c3ccccn3)Nc3cccc(n3)S(=O)(=O)NC(=O)c3ccc(-c4cccc(F)n4)nc3N1C2 ; CC1(C)C[C@@H]2CC[C@H](c3ccccn3)Nc3cccc(n3)S(=O)(=O)NC(=O)c3ccc(-c4cccc(OCC5CCC5)n4)nc3N1C2]

To a solution of cyclobutylmethanol (16 μL, 0.170 mmol) in DMF (550 μL) was added [bis(trimethylsilyl)amino]sodium (170 μL of 1 M in THF, 0.170 mmol) and after stirring at rt for 1 min, (14S,17R)-8-(6-fluoropyridin-2-yl)-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (20 mg, 0.034 mmol) was added. Then the mixture was stirred at 80° C. for 1 h, diluted with 5 mL DCM and then 0.2 mL of 1 N HCl and 5 mL water was added (pH paper <4). The mixture was partitioned, the aqueous layer extracted with DCM, the organics dried (MgSO₄) and evaporated. The residue was subjected to preparative SFC eluting a gradient of 5 mM NH₃ in methanol to CO₂ (15-50% over 10 min) though a 21.2×250 mm 2-PIC column, 5 μm particle, giving as a white solid the target: (14S,17R)-8-[6-(cyclobutylmethoxy)pyridin-2-yl]-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (10.3 mg, 46%). ¹H NMR (400 MHz, Chloroform-d) δ 8.56 (d, J=4.7 Hz, 1H), 8.13 (d, J=8.0 Hz, 1H), 7.93 (d, J=8.0 Hz, 1H), 7.86 (d, J=7.4 Hz, 1H), 7.68 (d, J=7.7 Hz, 1H), 7.66-7.63 (m, 1H), 7.60-7.51 (m, 2H), 7.31 (d, J=7.8 Hz, 1H), 7.20 (dd, J=7.6, 4.8 Hz, 1H), 6.77 (d, J=8.2 Hz, 1H), 6.67 (d, J=7.8 Hz, 1H), 5.94 (d, J=8.2 Hz, 1H), 5.38 (q, J=7.0 Hz, 1H), 4.39 (d, J 6.8 Hz, 2H), 3.52 (s, 1H), 3.05 (dd, J=8.9, 5.3 Hz, 2H), 2.82 (hept, J=7.7 Hz, 1H), 2.24 (dd, J=12.1, 7.4 Hz, 1H), 2.20-2.10 (m, 2H), 2.07-1.84 (m, 5H), 1.77 (s, 1H), 1.73 (s, 3H), 1.71 (s, 3H), 1.60 (dd, J=12.4, 7.9 Hz, 2H), 1.52 (q, J=7.4, 5.9 Hz, 1H). ESI-MS m/z calc. 653.27844, found 654.5 (M+1)⁺; Retention time: 1.47 minutes (LC method A).

The following is a list of alcohol reagents that are commercially available:
2-Methylpropan-1-ol
Cyclopropylmethanol
2,2-Dimethylpropan-1-ol
2-Cyclopropylethanol
3,3-Dimethylbutan-1-ol
Ethanol

The compounds in the following table were prepared in a manner analogous to that described above using alcohol reagents given in the table above:

| Compound | | |
|---|---|---|
| Number | Structure | NMR |
| 316 | 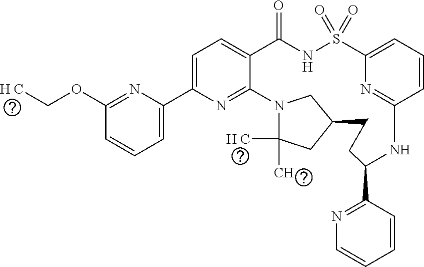 | ¹H NMR (400 MHz, Chloroform-d) δ 8.57 (d, J = 4.8 Hz, 1H), 8.12 (d, J = 8.0 Hz, 1H), 7.93 (d, J = 8.1 Hz, 1H), 7.87 (d, J = 7.5 Hz, 1H), 7.67 (q, J = 7.4, 7.0 Hz, 2H), 7.61-7.51 (m, 2H), 7.31 (d, J = 7.8 Hz, 1H), 7.21 (dd, J = 7.6, 4.9 Hz, 1H), 6.77 (d, J = 8.2 Hz, 1H), 6.67 (d, J = 7.8 Hz, 1H), 5.93 (d, J = 8.3 Hz, 1H), 5.38 (q, J = 7.1 Hz, 1H), 4.49 (q, J = 7.0 Hz, 2H), 3.52 (s, 1H), 3.11- 3.02 (m, 2H), 2.24 (dd, J = 12.3, 7.7 Hz, 1H), 1.94 (dt, J = 14.9, 6.9 Hz, 1H), 1.77 (d, J = 9.6 Hz, 1H), 1.73 (s, 3H), 1.71 (s, 3H), 1.68-1.49 (m, 3H), 1.45 (t, J = 7.1 Hz, 3H). |
| 310 | 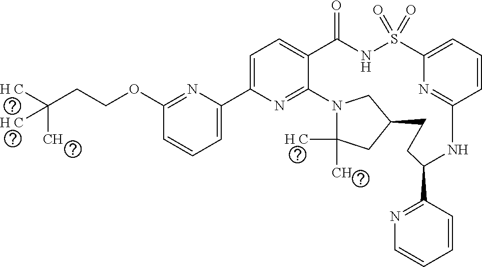 | ¹H NMR (400 MHz, Chloroform-d) δ 8.57 (d, J = 4.8 Hz, 1H), 8.13 (d, J = 8.0 Hz, 1H), 7.93 (d, J = 8.0 Hz, 1H), 7.86 (d, J = 7.4 Hz, 1H), 7.70- 7.63 (m, 2H), 7.62-7.52 (m, 2H), 7.31 (d, J = 7.8 Hz, 1H), 7.21 (dd, J = 7.6, 4.9 Hz, 1H), 6.75 (d, J = 8.2 Hz, 1H), 6.67 (d, J = 7.6 Hz, 1H), 5.94 (d, J = 8.2 Hz, 1H), 5.37 (q, J = 7.0 Hz, 1H), 4.50 (t, J = 7.4 Hz, 2H), 3.52 (s, 1H), 3.04 (d, J = 8.6 Hz, 2H), 2.25 (t, J = 10.3 Hz, 1H), 1.95 (dt, J = 14.5, 7.3 Hz, 1H), 1.76 (d, J = 7.4 Hz, 3H), 1.74 (s, 3H), 1.71 (s, 3H), 1.67-1.45 (m, 3H), 1.03 (s, 9H). |
| 311 | 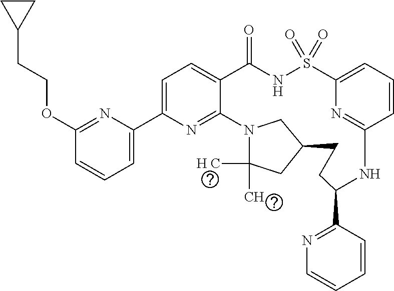 | ¹H NMR (400 MHz, Chloroform-d) δ 8.55 (d, J = 4.9 Hz, 1H), 8.11 (d, J = 8.0 Hz, 1H), 7.91 (d, J = 7.9 Hz, 1H), 7.86 (d, J = 7.4 Hz, 1H), 7.68 (d, J = 7.8 Hz, 1H), 7.65 (d, J = 7.3 Hz, 1H), 7.61-7.49 (m, 2H), 7.31 (d, J = 7.8 Hz, 1H), 7.19 (dd, J = 7.5, 5.0 Hz, 1H), 6.76 (d, J = 8.2 Hz, 1H), 6.66 (d, J = 8.0 Hz, 1H), 5.91 (s, 1H), 5.38 (q, J = 7.1 Hz, 1H), 4.49 (t, J = 6.8 Hz, 2H), 3.52 (s, 1H), 3.02 (d, J = 7.8 Hz, 2H), 2.22 (d, J = 11.2 Hz, 1H), 1.94 (dt, J = 15.1, 7.2 Hz, 1H), 1.83-1.68 (m, 9H), 1.66-1.56 (m, 2H), 1.48 (dd, J = 22.3, 8.3 Hz, 1H), 0.86 (ddt, J = 10.8, 7.8, 3.8 Hz, 1H), 0.53-0.46 (m, 2H), 0.14 (t, J = 5.0 Hz, 2H). |
| 312 | 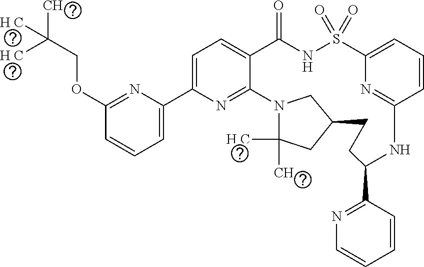 | ¹H NMR (400 MHz, Chloroform-d) δ 8.56 (d, J = 4.8 Hz, 1H), 8.13 (d, J = 8.0 Hz, 1H), 7.93 (d, J = 8.0 Hz, 1H), 7.85 (d, J = 7.4 Hz, 1H), 7.72- 7.63 (m, 2H), 7.61-7.53 (m, 2H), 7.31 (d, J = 7.8 Hz, 1H), 7.20 (dd, J = 7.6, 4.9 Hz, 1H), 6.79 (d, J = 8.2 Hz, 1H), 6.67 (d, J = 7.7 Hz, 1H), 5.95 (d, J = 8.2 Hz, 1H), 5.37 (q, J = 7.1 Hz, 1H), 4.10 (s, 2H), 3.52 (s, 1H), 3.04 (d, J = 7.6 Hz, 1H), 2.24 (t, J = 10.0 Hz, 1H), 2.00-1.89 (m, 1H), 1.77 (d, J = 12.3 Hz, 1H), 1.73 (s, 3H), 1.71 (s, 3H), 1.68-1.56 (m, 2H), 1.53 (d, J = 8.8 Hz, 2H), 1.07 (s, 9H). |
| 314 | 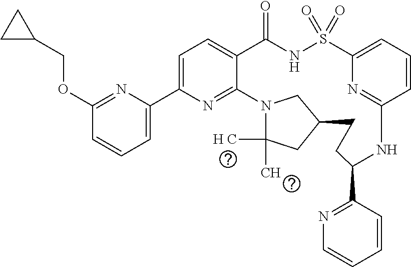 | ¹H NMR (400 MHz, Chloroform-d) δ 8.56 (d, J = 4.7 Hz, 1H), 8.11 (d, J = 8.0 Hz, 1H), 7.91-7.84 (m, 2H), 7.73-7.64 (m, 2H), 7.58 (t, J = 7.7 Hz, 1H), 7.53 (d, J = 7.2 Hz, 1H), 7.31 (d, J = 7.8 Hz, 1H), 7.21 (dd, J = 7.5, 4.9 Hz, 1H), 6.81 (d, J = 8.2 Hz, 1H), 6.67 (d, J = 8.0 Hz, 1H), 5.88 (d, J = 8.3 Hz, 1H), 5.38 (q, J = 7.1 Hz, 1H), 4.26 (d, J = 7.2 Hz, 2H), 3.51 (s, 1H), 3.02 (q, J = 15.3, 12.9 Hz, 2H), 2.22 (dd, J = 12.2, 7.4 Hz, 1H), 1.94 (dt, J = 14.6, 7.2 Hz, 1H), 1.88-1.75 (m, 1H), 1.73 (s, 3H), 1.71 (s, 3H), 1.60 (dd, J = 11.7, 8.4 Hz, 2H), 1.56-1.45 (m, 1H), 1.36 (ddt, J = 12.5, 7.8, 3.8 Hz, 1H), |
| | | 0.67-0.60 (m, 2H), 0.41 (t, J = 5.0 |
| | | Hz, 2H). |
| 315 | 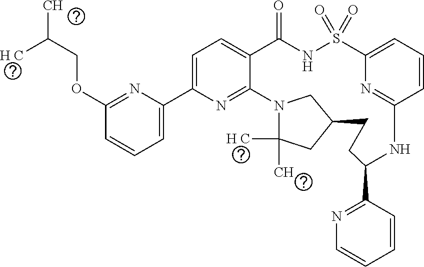 | ¹H NMR (400 MHz, Chloroform-d) δ 8.57 (d, J = 4.8 Hz, 1H), 8.13 (d, J = 8.0 Hz, 1H), 7.93 (d, J = 8.0 Hz, 1H), 7.86 (d, J = 7.4 Hz, 1H), 7.74- 7.62 (m, 2H), 7.62-7.51 (m, 2H), 7.31 (d, J = 7.9 Hz, 1H), 7.21 (dd, J = 7.5, 4.9 Hz, 1H), 6.78 (d, J = 8.2 Hz, 1H), 6.70-6.63 (m, 1H), 5.93 (d, J = 8.2 Hz, 1H), 5.37 (q, J = 7.0 Hz, 1H), 4.19 (d, J = 6.6 Hz, 2H), 3.52 (s, 1H), 3.04 (d, J = 8.2 Hz, 2H), 2.25 (dd, J = 12.3, 8.0 Hz, 1H), 2.14 (dq, J = 13.4, 6.7 Hz, 1H), 1.95 (dt, J = 14.5, 7.3 Hz, 1H), 1.89- 1.78 (m, 1H), 1.73 (s, 3H), 1.71 (s, 3H), 1.67-1.57 (m, 2H), 1.51 (q, J = 7.5, 6.1 Hz, 1H), 1.05 (d, J = 6.7 |
| | | Hz, 6H). |

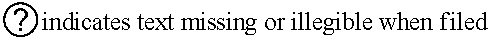

| Compound | LCMS Retention | Exact | | |
|---|---|---|---|---|
| Number | Time (min) | Mass | M + 1 | LCMS method |
| 316 | 1.5 | 613.247 | 614.3 | LC method A |
| 310 | 1.62 | 669.31 | 670.7 | LC method A |
| 311 | 1.4 | 653.278 | 654.2 | LC method A |
| 312 | 1.51 | 655.294 | 656.6 | LC method A |
| 314 | 1.3 | 639.263 | 640.3 | LC method A |
| 315 | 1.42 | 641.278 | 642.4 | LC method A |

The compounds in the following table were prepared in a manner analogous to that described above using pyrazole reagents prepared in the examples above, pyrazole, alcohol and boronic acid reagents given in the tables above and using (14S,17S)-8-bromo-12,12-dimethyl-17-(pyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione as a starting material:

| Compound | | |
|---|---|---|
| Number | Structure | NMR |
| 309 | 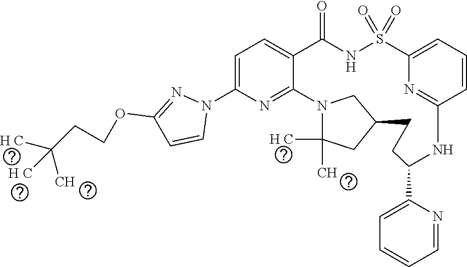 | ¹H NMR (400 MHz, Chloroform-d) δ 8.58 (d, J = 4.8 Hz, 1H), 8.25 (d, J = 8.5 Hz, 1H), 8.19 (d, J = 2.8 Hz, 1H), 7.71 (td, J = 7.7, 1.8 Hz, 1H), 7.66- 7.56 (m, 2H), 7.44 (d, J = 8.5 Hz, 1H), 7.33-7.20 (m, 2H), 6.82-6.70 (m, 2H), 5.93 (d, J = 2.8 Hz, 1H), 4.79 (s, 1H), 4.32 (t, J = 7.4 Hz, 2H), 3.54 (dd, J = 10.1, 4.4 Hz, 1H), 3.32 (s, 1H), 2.36 (s, 2H), 2.11-2.04 (m, 1H), 1.95 (t, J = 8.9 Hz, 1H), 1.85 (d, J = 9.0 Hz, 2H), 1.79 (d, J = 10.8 Hz, 1H), 1.74 (t, J = 7.4 Hz, 2H), 1.61 (d, J = 2.3 Hz, 6H), 1.00 (s, 9H). |
| 338 | 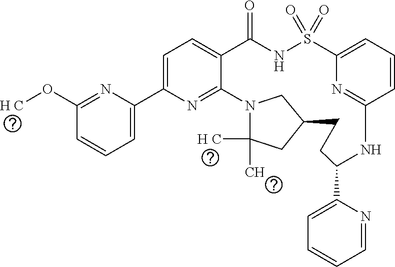 | ¹H NMR (400 MHz, Chloroform-d) δ 11.90 (s, 1H), 8.58 (d, J = 4.8 Hz, 1H), 8.33 (d, J = 8.1 Hz, 1H), 8.13 (d, J = 8.1 Hz, 1H), 7.89 (d, J = 7.4 Hz, 1H), 7.73-7.54 (m, 4H), 7.28 (d, J = 7.9 Hz, 1H), 7.25-7.16 (m, 1H), 6.79 (dd, J = 13.0, 8.1 Hz, 3H), 4.78 (s, 1H), 4.03 (s, 3H), 3.50 (d, J = 8.1 Hz, 1H), 3.33 (d, J = 8.6 Hz, 1H), 2.52 (s, 1H), 2.38 (s, 1H), 2.16-2.08 (m, 1H), 1.99 (s, 1H), 1.83 (q, J = 11.3 Hz, 2H), 1.69 (s, 3H), 1.65 (s, 3H), 1.54 (d, J = 21.2 Hz, 1H). |
| 344 | 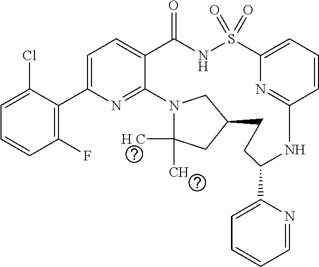 | ¹H NMR (400 MHz, Chloroform-d) δ 11.71 (s, 1H), 8.59 (d, J = 4.9 Hz, 1H), 8.27 (d, J = 7.9 Hz, 1H), 7.74 (s, 1H), 7.65 (d, J = 7.4 Hz, 1H),7.61 (t, J = 7.3 Hz, 1H), 7.33 (dd, J = 8.1, 5.6 Hz, 1H), 7.30-7.27 (m, 2H), 7.09 (ddd, J = 9.3, 7.7, 1.7 Hz, 2H), 6.79 (d, J = 1.4 Hz, 1H), 6.77 (d, J = 1.4 Hz, 1H), 4.83 (s, 1H),3.51 (dd, J = 9.9, 4.6Hz, 1H), 3.37 (s, 1H), 2.37 (s, 2H), 2.04 (dd, J = 12.2, 7.2 Hz, 1H), 1.94 (dd, J = 19.2, 11.8 Hz, 3H), 1.77 (t, J = 10.7 Hz, 1H), 1.56 (s, 4H), 1.50 (s, 3H). |
| 340 | 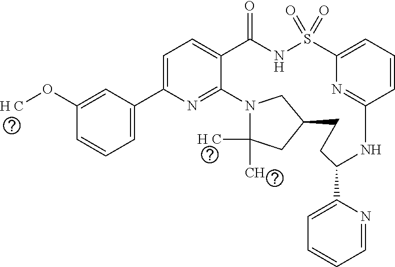 | ¹H NMR (400 MHz, Chloroform-d) δ 11.78 (s, 1H), 8.57 (s, 1H), 8.26 (d, J = 8.1 Hz, 1H),7.63 (td, J =22.7, 11.3 Hz, 5H), 7.47 (d, J = 8.0 Hz, 1H), 7.38 (t, J = 7.9 Hz, 1H), 7.28 (d, J = 7.9 Hz, 1H), 7.24 (s, 1H), 6.99 (d, J = 8.3 Hz, 1H), 6.77 (d, J = 7.6 Hz, 2H), 4.78 (s, 1H), 3.87 (s, 3H), 3.51 (s, 1H), 3.36 (s, 1H), 2.49 (s, 1H), 2.38 (s, 1H), 2.11 (d, J = 11.4 Hz, 1H), 1.98 (s, 1H), 1.82 (d, J = 12.0 Hz, 2H), 1.69 (s, 3H), 1.66 (s, 3H), 1.54 (d, J = 30.6 Hz, 1H). |
| 348 | 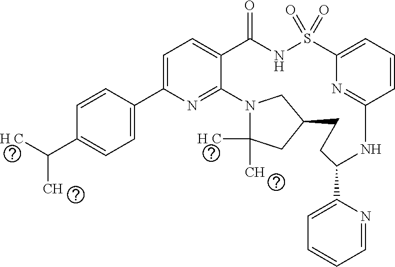 | ¹H NMR (400 MHz, Chloroform-d) δ 11.86 (s, 1H), 8.66-8.52 (m, 1H), 8.25 (d, J = 8.1 Hz, 1H), 8.01-7.91 (m, 2H), 7.74 (t, J =7.8 Hz, 1H), 7.68-7.57 (m, 2H), 7.45 (d, J = 8.2 Hz, 1H), 7.34 (d, J = 8.3 Hz, 2H), 7.27 (d, J = 7.2 Hz, 1H), 6.82 (s, 1H), 6.79-6.75 (m, 1H), 4.82 (s, 1H), 3.49 (dd, J = 9.8, 4.9 Hz, 1H), 3.35 (s, 1H), 2.97 (hept, J = 7.0 Hz, 1H), 2.43 (m, 2H), 2.10 (dd, J = 12.1, 7.5 Hz, 1H), 2.05-1.94 (m, 1H), 1.91-1.74 (m, 4H), 1.69 (s, 3H), 1.65 (s, 3H), 1.29 (d, J = 6.9 Hz, 6H). |
| 342 | 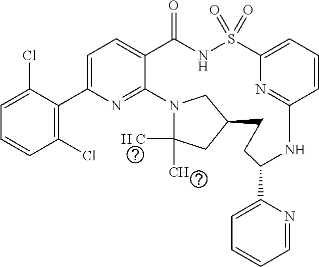 | ¹H NMR (400 MHz, Chloroform-d) δ 11.65 (s, 1H), 8.62-8.52 (m, 1H), 8.29 (d, J = 7.9 Hz, 1H), 7.71 (dd, J = 7.5 Hz, 1H), 7.63 (dd, J = 15.0, 7.1 Hz, 2H), 7.40 (s, 1H), 7.38 (s, 1H), 7.29 (d, J = 8.0 Hz, 2H), 7.23-7.20 (m, 1H), 7.01 (d, J = 7.9 Hz, 1H), 6.78-6.76 (m, 2H), 4.79 (s, 1H), 3.51 (dt, J = 9.4, 4.8 Hz, 1H), 3.36 (s, 1H), 2.37 (s, 2H), 2.09- 1.90 (m, 2H), 1.87 (d, J = 9.7 Hz, 1H), 1.77 (t, J = 10.8 Hz, 1H), 1.63 (s, 1H), 1.54 (s, 3H), 1.48 (s, 3H). |
| 345 | 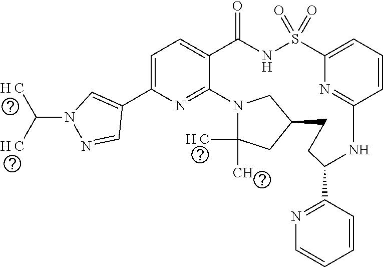 | ¹H NMR (400 MHz, Chloroform-d) δ 11.88 (s, 1H), 8.70-8.45 (m, 1H), 8.17 (d, J = 8.1 Hz, 1H), 7.99 (s, 1H), 7.89 (s, 1H), 7.71 (td, J = 7.7, 1.8 Hz, 1H), 7.65 (d, J = 7.l Hz, 1H), 7.61 (t, J = 7.7 Hz, 2H), 7.29 (d, J = 7.8 Hz, 1H), 7.24 (dd, J = 7.6, 5.1 Hz, 1H), 7.15 (d, J = 8.2 Hz, 1H), 6.76 (d, J = 8.1 Hz, 2H), 4.77 (s, 1H), 4.54 (hept, J = 6.7 Hz, 1H), 3.47 (dd, J = 10.0, 5.0 Hz, 1H), 3.32 (s, 1H), 2.51 (s, 1H), 2.36 (s, 1H), 2.15-2.03 (m, 1H), 1.97 (t, J = 8.8 Hz, 1H), 1.82 (dt, J = 21.7, 10.0 Hz, 3H), 1.66 (s, 3H), 1.61 (s, 3H), 1.56 (d, J = 6.7 Hz, 6H). |
| 317 | 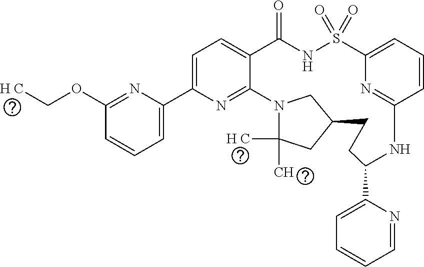 | ¹H NMR (400 MHz, Chloroform-d) δ 11.4 (brs, 1H), 8.58 (d, J = 4.8 Hz, 1H), 8.33 (d, J = 8.2 Hz, 1H), 8.10 (d, J = 8.1 Hz, 1H), 7.87 (d, J = 7.4 Hz, 1H), 7.67 (ddt, J = 23.2, 15.4, 7.5 Hz, 4H), 7.29 (d, J = 7.9 Hz, 1H), 7.24 (d, J = 8.3 Hz, 1H), 6.84-6.71 (m, 3H), 4.77 (s, 1H), 4.48 (q, J = 7.1 Hz, 2H), 3.50 (dd, J = 10.0, 4.9 Hz, 1H), 3.34 (s, 1H), 2.53 (s, 1H), 2.38 (s, 1H), 2.12 (dd, J = 12.1, 7.9 Hz, 1H), 1.97 (d, J = 13.2 Hz, 1H), 1.83 (q, J = 11.1 Hz, 3H), 1.69 (s, 3H), 1.65 (s, 3H), 1.44 (t, J = 7.1 Hz, 3H). |
| 350 | 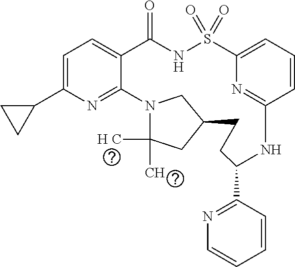 | ¹H NMR (400 MHz, Chloroform-d) δ 11.82 (s, 1H), 8.58 (d, J = 4.9 Hz, 1H), 8.05 (d, J = 8.0 Hz, 1H), 7.74 (t, J = 7.8 Hz, 1H), 7.66-7.55 (m, 2H), 7.34 (s, 1H), 6.92 (d, J = 8.0 Hz, 1H), 6.82 (s, 1H), 6.76 (d, J = 7.8 Hz, 1H), 4.79 (s, 1H), 3.39 (dd, J = 9.9, 5.1 Hz, 1H), 3.23 (s, 1H), 2.46 (s, 1H), 2.34 (d, J = 12.0 Hz, 1H), 2.17-2.00 (m, 1H), 1.98-1.91 (m, 1H), 1.86-1.82 (m, 1H), 1.74 (t, J = 11.0 Hz, 1H), 1.69-1.56 (m, 1H), 1.51 (s, 6H), 1.12-0.97 (m, 4H), 0.92-0.78 (m, 2H). |

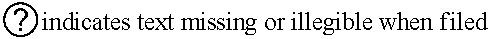

| Compound | LCMS Retention | Exact | | |
|---|---|---|---|---|
| Number | Time (min) | Mass | M + 1 | LCMS method |
| 309 | 1.6 | 658.305 | 659.5 | LC method A |
| 338 | 0.97 | 599.231 | 600.4 | LC method A |
| 344 | 1.27 | 620.177 | 621.1 | LC method A |
| 340 | 1.24 | 598.236 | 599.4 | LC method A |
| 348 | 1.48 | 610.273 | 611.1 | LC method A |
| 342 | 1.35 | 636.148 | 637.3 | LC method A |
| 345 | 1.01 | 600.263 | 601.1 | LC method A |
| 317 | 1.36 | 613.247 | 614.3 | LC method A |
| 350 | 1.05 | 532.226 | 533 | LC method A |

### Example 111: Preparation of (14S,17R)-8-bromo-17-(4-tert-butylpyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione and (14S,17S)-8-bromo-17-(4-tert-butylpyridin-2-yl)-12,12-dimethyl-2λ⁶-thia-3,9,11,18,23-pentaazatetracyclo [17.3.1.111,14.05,10]tetracosa-1(22),5,7,9,19(23),20-hexaene-2,2,4-trione

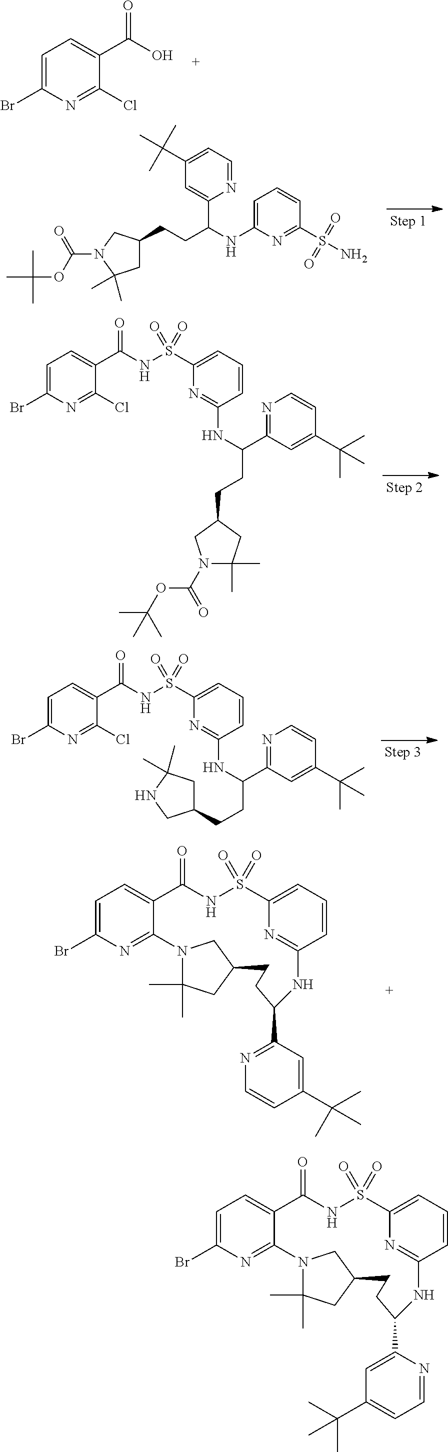

### Step 1: tert-Butyl (4S)-4-[3-[[6-[(6-bromo-2-chloro-pyridine-3-carbonyl)sulfamoyl]-2-pyridyl]amino]-3-(4-tert-butyl-2-pyridyl)propyl]-2,2-dimethyl-pyrrolidine-1-carboxylate

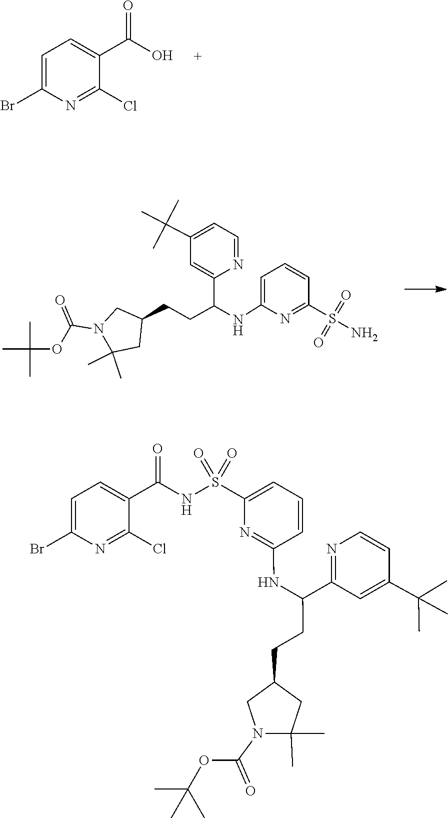 [SMILES: O=C(O)c1ccc(Br)nc1Cl ; CC(C)(C)OC(=O)N1C[C@@H](CCC(Nc2cccc(S(N)(=O)=O)n2)c2cc(C(C)(C)C)ccn2)CC1(C)C ; CC(C)(C)OC(=O)N1C[C@@H](CCC(Nc2cccc(S(=O)(=O)NC(=O)c3ccc(Br)nc3Cl)n2)c2cc(C(C)(C)C)ccn2)CC1(C)C]

A mixture of 6-bromo-2-chloro-pyridine-3-carboxylic acid (510 mg, 2.157 mmol) and CDI (350 mg, 2.159 mmol) were dissolved in THF (3.6 mL) and the mixture stirred at 60° C. for 45 min then tert-butyl (4S)-4-[3-(4-tert-butyl-2-pyridyl)-3-[(6-sulfamoyl-2-pyridyl)amino]propyl]-2,2-dimethyl-pyrrolidine-1-carboxylate (587 mg, 1.076 mmol) was added followed by DBU (725 μL, 4.848 mmol) and the resulting mixture was stirred at rt for 2h. The mixture was diluted with EtOAc and washed with 1 N HCl in water, water, brine, dried (MgSO₄), and concentrated. The residue was purified (flash chromatography: 40 g SiO₂, 10-80% EtOAc in hexanes, 20 min) to provide the desired product: tert-Butyl (4S)-4-[3-[[6-[(6-bromo-2-chloro-pyridine-3-carbonyl)sulfamoyl]-2-pyridyl]amino]-3-(4-tert-butyl-2-pyridyl)propyl]-2,2-dimethyl-pyrrolidine-1-carboxylate (707 mg, 86%). ¹H NMR (400 MHz, Chloroform-d) δ 8.54 (d, J=5.4 Hz, 1H), 7.78 (d, J=8.5 Hz, 1H), 7.58-7.46 (m, 2H), 7.43 (d, J=7.8 Hz, 1H), 7.34 (s, 1H), 7.29 (s, 1H), 6.62 (s, 1H), 5.94-5.81 (m, 1H), 5.12 (m, 1H), 4.24-4.09 (m, 1H), 3.05 (d, J=11.5 Hz, 1H), 2.85 (t, J=10.8 Hz, 1H), 2.41 (s, 1H), 2.17 (s, 2H), 1.95-1.68 (m, 4H), 1.41 (d, J=4.2 Hz, 9H), 1.35 (s, 5H), 1.33 (s, 4H), 1.32-1.24 (m, 4H). ESI-MS m/z calc. 762.1966, found 764.9 (M+1)⁺; Retention time: 0.64 minutes (LC method D).

### Step 2: 6-Bromo-N-[[6-[[1-(4-tert-butyl-2-pyridyl)-3-[(3R)-5,5-dimethylpyrrolidin-3-yl]propyl]amino]-2-pyridyl]sulfonyl]-2-chloro-pyridine-3-carboxamide

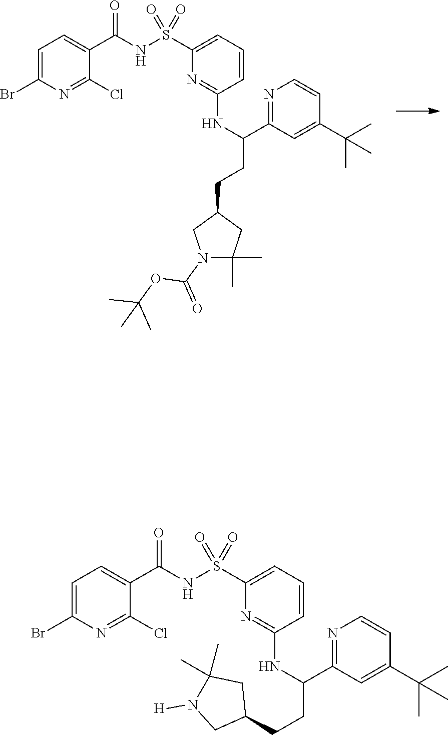 [SMILES: CC(C)(C)OC(=O)N1C[C@@H](CCC(Nc2cccc(S(=O)(=O)NC(=O)c3ccc(Br)nc3Cl)n2)c2cc(C(C)(C)C)ccn2)CC1(C)C ; CC1(C)C[C@@H](CCC(Nc2cccc(S(=O)(=O)NC(=O)c3ccc(Br)nc3Cl)n2)c2cc(C(C)(C)C)ccn2)CN1]

To a solution of tert-butyl (4S)-4-[3-[[6-[(6-bromo-2-chloro-pyridine-3-carbonyl)sulfamoyl]-2-pyridyl]amino]-3-(4-tert-butyl-2-pyridyl)propyl]-2,2-dimethyl-pyrrolidine-1-carboxylate (707 mg, 0.925 mmol) in DCM (5 mL) was added TFA (2.2 mL, 28.56 mmol) and the mixture was stirred at rt for 45 min. The solution was concentrated to dryness under reduced pressure, then co-evaporated with THF (2×20 mL), then dried under high vacuum at rt for 16h to provide: 6-bromo-N-[[6-[[1-(4-tert-butyl-2-pyridyl)-3-[(3R)-5,5-dimethylpyrrolidin-3-yl]propyl]amino]-2-pyridyl]sulfonyl]-2-chloro-pyridine-3-carboxamide (trifluoroacetate salt) (984 mg, 106%). ESI-MS m/z calc. 662.14417, found 665.0 (M+1)⁺; Retention time: 0.39 minutes (LC method D).

### Step 3: (14S,17R)-8-bromo-17-(4-tert-butylpyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, and (14S,17S)-8-bromo-17-(4-tert-butylpyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione

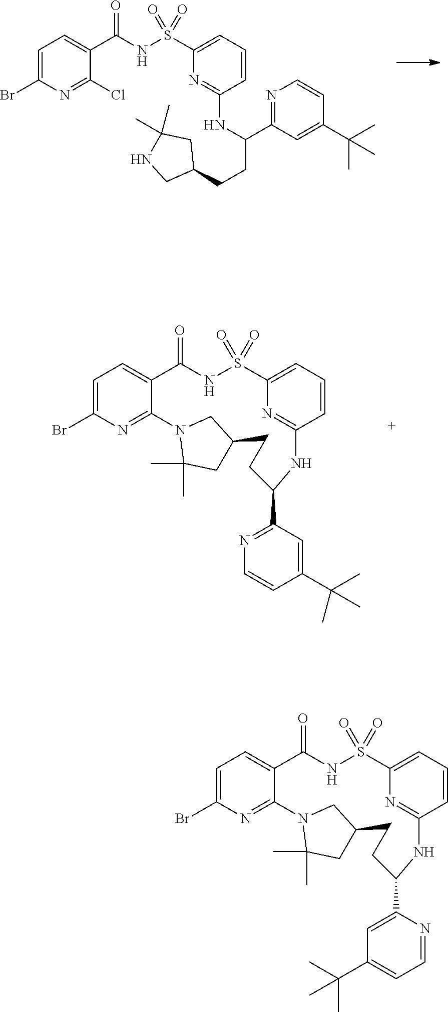 [SMILES: CC1(C)C[C@@H](CCC(Nc2cccc(S(=O)(=O)NC(=O)c3ccc(Br)nc3Cl)n2)c2cc(C(C)(C)C)ccn2)CN1 ; CC(C)(C)c1ccnc([C@H]2CC[C@@H]3CN(c4nc(Br)ccc4C(=O)NS(=O)(=O)c4cccc(n4)N2)C(C)(C)C3)c1 ; CC(C)(C)c1ccnc([C@@H]2CC[C@@H]3CN(c4nc(Br)ccc4C(=O)NS(=O)(=O)c4cccc(n4)N2)C(C)(C)C3)c1]

A mixture of 6-bromo-N-[[6-[[1-(4-tert-butyl-2-pyridyl)-3-[(3R)-5,5-dimethylpyrrolidin-3-yl]propyl]amino]-2-pyridyl]sulfonyl]-2-chloro-pyridine-3-carboxamide (trifluoroacetate salt) (984 mg, 0.978 mmol) and potassium carbonate (811 mg, 5.868 mmol) and potassium carbonate (811 mg, 5.868 mmol) in DMA (10 mL) was heated at 140° C. for 7h, cooled by ice bath, diluted with 100 mL of EtOAc and 30 mL of water and then 15 mL of 1 M HCl (2.5×the amount of K₂CO₃) which produced a mixture with a pH 2 by paper. The mixture was partitioned, and the organic layer separated. The aqueous layer was extracted with 100 mL of EtOAc. The combined organic extracts were dried (MgSO₄) and evaporated to an oil. The residue was dissolved into DMSO (100 mg/mL) and diluted with 1 volume of DMA. This solution was purified with preparative SFC eluting a gradient of 5 mM NH₃ in methanol to CO₂ (40-70% over 10 min) though a 21.2×250 mm 2-PIC column, 5 μm particle giving as a white solid (after evaporation of the collected fractions):

First to elute: (14S,17R)-8-bromo-17-(4-tert-butylpyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (139 mg, 23%). ¹H NMR (400 MHz, Chloroform-d) δ 8.35 (d, J=5.3 Hz, 1H), 7.62 (d, J=7.9 Hz, 1H), 7.54 (t, J=7.8 Hz, 1H), 7.43 (d, J=7.3 Hz, 1H), 7.16 (d, J=5.3 Hz, 1H), 6.76 (d, J=7.9 Hz, 1H), 6.64 (d, J=8.4 Hz, 1H), 5.56 (s, 1H), 5.35 (q, J=7.9 Hz, 1H), 3.47 (s, 1H), 2.56 (s, 1H), 1.98 (dd, J=12.1, 6.8 Hz, 1H), 1.90 (s, 2H), 1.62 (s, 3H), 1.60 (s, 2H), 1.56 (s, 3H), 1.48 (d, J 28.3 Hz, 3H), 1.29 (s, 9H). ESI-MS m/z calc. 626.1675, found 625.3 (M+1)⁺; Retention time: 1.28 minutes (LC method A).

Second to elute: (14S,17S)-8-bromo-17-(4-tert-butylpyridin-2-yl)-12,12-dimethyl-2λ⁶-thia-3,9,11,18,23-pentanzatetracyclo[17.3.1.111,14.05,10]tetracosa-1(22),5,7,9,19(23),20-hexaene-2,2,4-trione (145 mg, 24%). ¹H NMR (400 MHz, Chloroform-d) δ 8.48 (d, J=5.2 Hz, 1H), 7.84 (s, 1H), 7.62-7.54 (m, 2H), 7.24 (d, J=5.7 Hz, 1H), 6.99 (s, 1H), 6.71 (s, 1H), 6.50 (s, 1H), 4.83 (s, 1H), 3.59 (s, 1H), 3.26 (s, 1H), 2.33 (s, 1H), 1.99 (s, 2H), 1.85 (s, 2H), 1.71 (t, J=11.6 Hz, 1H), 1.59 (s, 3H), 1.52 (s, 3H), 1.46 (d, J=27.1 Hz, 1H), 1.34 (s, 9H), 1.32-1.29 (m, 1H). ESI-MS m/z calc. 626.1675, found 627.3 (M+1)⁺; Retention time: 1.35 minutes (LC method A).

### Example 112: Preparation of (14S,17R)-17-(4-tert-butylpyridin-2-yl)-8-[3-(3,3-dimethylbutoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (Compound 305)

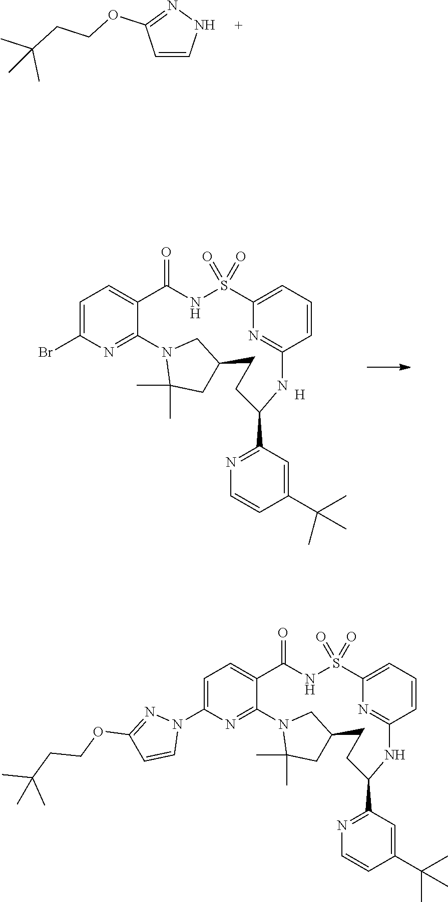

A mixture of 3-(3,3-dimethylbutoxy)-1H-pyrazole (89 mg, 0.529 mmol), hexaphenyl-1,7-dipalladanonacyclo[5.5.5.01,4.01,11.01,14.02,4.05,7.07,9.07,16]heptadeca-2,5,8,11,13,16-hexaene-4,10,15-trione (6 mg, 0.0066 mmol), (5-diphenylphosphanyl-9,9-dimethyl-xanthen-4-yl)-diphenyl-phosphane (12 mg, 0.021 mmol), Cs₂CO₃ (172 mg, 0.528 mmol) and DMF (1.3 mL) was degassed by bubbling nitrogen for 1 min then heated to 100° C. for 16 h. Then the mixture was diluted with 2 mL water, 2 mL of DCM, then 0.6 mL of 1 M HCl added and the mixture partitioned, when paper showed pH 4. The layers were separated and the aqueous layer extracted with DCM (2 mL). The combined organic extracts were dried (MgSO₄) and evaporated. The residue was dissolved in 1:1 MeOH/ACN at 40 mg/mL and subjected to preparative SFC eluting a gradient of 5 mM NH₃ in methanol to CO₂ (15-50% over 10 min) though a 21.2×250 mm 2-PIC column, 5 μm particle, giving as a white solid the target: (14S,17R)-17-(4-tert-butylpyridin-2-yl)-8-[3-(3,3-dimethylbutoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (28.2 mg, 30%). ¹H NMR (400 MHz, Chloroform-d) δ 8.46 (d, J=5.3 Hz, 1H), 8.19 (d, J=2.8 Hz, 1H), 8.06 (d, J=8.3 Hz, 1H), 7.61-7.49 (m, 2H), 7.29 (s, 1H), 7.20 (d, J=5.3 Hz, 1H), 6.65 (d, J=8.0 Hz, 1H), 5.91 (s, 1H), 5.85 (s, 1H), 5.30 (s, 3H), 4.32 (t, J=7.4 Hz, 2H), 3.53 (s, 1H), 3.01 (s, 2H), 2.21 (s, 1H), 1.92 (d, J=10.1 Hz, 1H), 1.74 (t, J=7.4 Hz, 3H), 1.65 (s, 6H), 1.58 (d, J=9.5 Hz, 1H), 1.49 (d, J=11.6 Hz, 1H), 1.32 (s, 9H), 1.00 (s, 9H). ESI-MS m/z calc. 714.36755, found 715.7 (M+1)⁺; Retention time: 1.72 minutes (LC method A).

### Example 113: Preparation of (14S,17R)-17-(4-tert-butylpyridin-2-yl)-8-(6-fluoropyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (Compound 307)

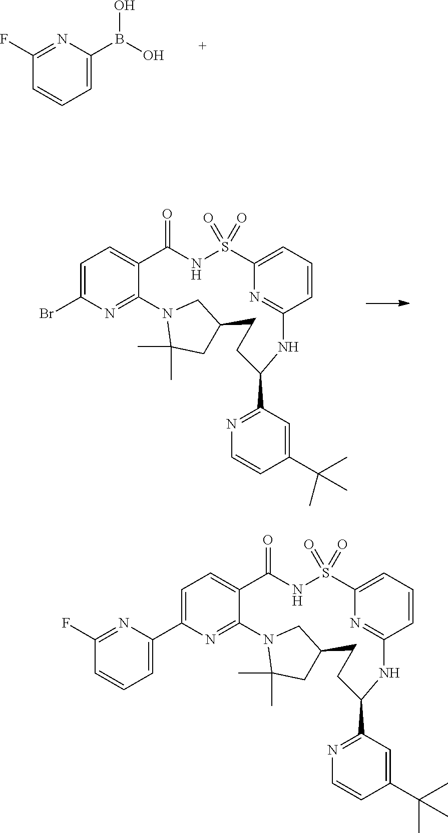

A mixture of (14S,17R)-8-bromo-17-(4-tert-butylpyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (55 mg, 0.088 mmol), (6-fluoro-2-pyridyl)boronic acid (37 mg, 0.26 mmol), Pd(dppf)Cl₂ (6.4 mg, 0.0087 mmol), acetonitrile (968 μL) and NaHCO₃(520 μL of 1 M, 0.52 mmol) was degassed by bubbling nitrogen for 1 min then heated to 100° C. for 2 h. Then the mixture was diluted with 2 mL water, 2 mL of 5:1 DCM/MeOH, then 0.5 mL of 1 M HCl added and the mixture partitioned, when paper showed pH 4. The layers were separated and the aqueous layer extracted with 5:1 DCM/MeOH (2 mL). The residue was purified by flash chromatography (4 g SiO₂, 20-80% EtOAc in hexanes over 15 min) to provide the target: (14S,17R)-17-(4-tert-butylpyridin-2-yl)-8-(6-fluoropyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (25.5 mg, 45%). ¹H NMR (400 MHz, Chloroform-d) δ 8.46 (d, J=5.2 Hz, 1H), 8.22-8.16 (m, 1H), 8.13 (d, J=8.0 Hz, 1H), 7.96-7.85 (m, 2H), 7.62-7.52 (m, 2H), 7.21-7.17 (m, 1H), 6.97 (dd, J=8.3, 2.8 Hz, 1H), 6.67 (d, J=7.8 Hz, 1H), 5.90 (d, J 8.2 Hz, 1H), 5.33 (q, J=7.4 Hz, 1H), 3.85-3.70 (m, 1H), 3.53 (s, 1H), 3.03 (s, 2H), 2.25 (t, J=9.6 Hz, 1H), 2.00-1.89 (m, 1H), 1.88-1.83 (m, 1H), 1.78 (d, J=7.5 Hz, 1H), 1.73 (s, 3H), 1.72 (s, 3H), 1.61 (q, J=9.4 Hz, 1H), 1.50 (d, J=16.1 Hz, 1H), 1.32 (s, 9H). ESI-MS m/z calc. 643.2741, found 644.6 (M+1)⁺; Retention time: 1.33 minutes (LC method A).

### Example 114: Preparation of (14S,17R)-17-(4-tert-butylpyridin-2-yl)-8-[6-(3,3-dimethylbutoxy)pyridin-2-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (Compound 303)

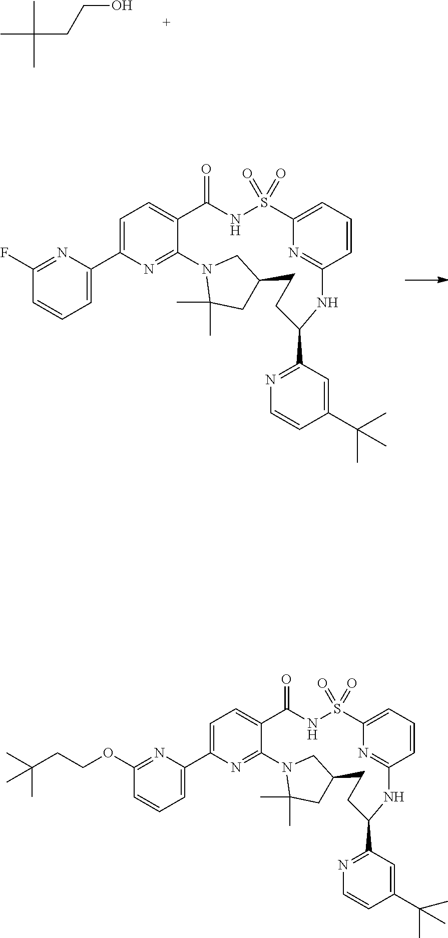

To a solution of 3,3-dimethylbutan-1-ol (360 μL, of 0.25 M, 0.09 mmol) in DMF was added [bis(trimethylsilyl)amino]sodium (90 μL, of 1 M, 0.09 mmol) and after stirring at rt for 1 min, it was added to (14S,17R)-17-(4-tert-butylpyridin-2-yl)-8-(6-fluoropyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (11.6 mg, 0.018 mmol). Then the mixture was stirred at 80° C. for 1 h, diluted with 5 mL DCM and then 0.2 mL of 1 N HCl and 5 mL water was added (pH paper <4). The mixture was partitioned, the aqueous layer extracted with DCM, the organics dried (MgSO₄) and evaporated. The residue was subjected to preparative SFC eluting a gradient of 5 mM NH₃ in methanol to CO₂ (15-50% over 10 min) though a 21.2×250 mm 2-PIC column, 5 μm particle, giving as a white solid the target: (14S,17R)-17-(4-tert-butylpyridin-2-yl)-8-[6-(3,3-dimethylbutoxy)pyridin-2-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (6.7 mg, 51%). ¹H NMR (400 MHz, Chloroform-d) δ 8.46 (d, J=5.3 Hz, 1H), 8.13 (s, 1H), 7.93 (s, 1H), 7.85 (d, J=7.4 Hz, 1H), 7.67 (t, J=7.8 Hz, 1H), 7.56 (d, J=6.5 Hz, 2H), 7.26 (s, 1H), 7.20 (d, J=5.2 Hz, 1H), 6.74 (d, J=8.2 Hz, 1H), 6.68-6.61 (m, 1H), 5.94 (s, 1H), 5.34 (d, J=6.9 Hz, 1H), 4.49 (t, J=7.4 Hz, 2H), 3.54 (s, 1H), 3.05-2.98 (m, 1H), 2.26 (s, 1H), 1.93 (d, J=9.0 Hz, 1H), 1.78-1.75 (m, 4H), 1.73 (s, 3H), 1.71 (s, 3H), 1.60 (d, J=12.4 Hz, 2H), 1.49 (d, J=9.1 Hz, 1H), 1.32 (s, 9H), 1.02 (s, 9H). ESI-MS m/z calc. 725.3723, found 726.5 (M+1)⁺; Retention time: 1.75 minutes (LC method A).

### Example 115: Preparation of (14S,17R)-17-(4-tert-butylpyridin-2-yl)-8-[6-(cyclobutyl methoxy)pyridin-2-yl]-12,12-dimethyl-2λ⁶-thia-3,9,11,18,23-pentaazatetracyclo [17.3.1.111,14.05,10]tetracosa-1(22),5,7,9,19(23),20-hexaene-2,2,4-trione (Compound 302)

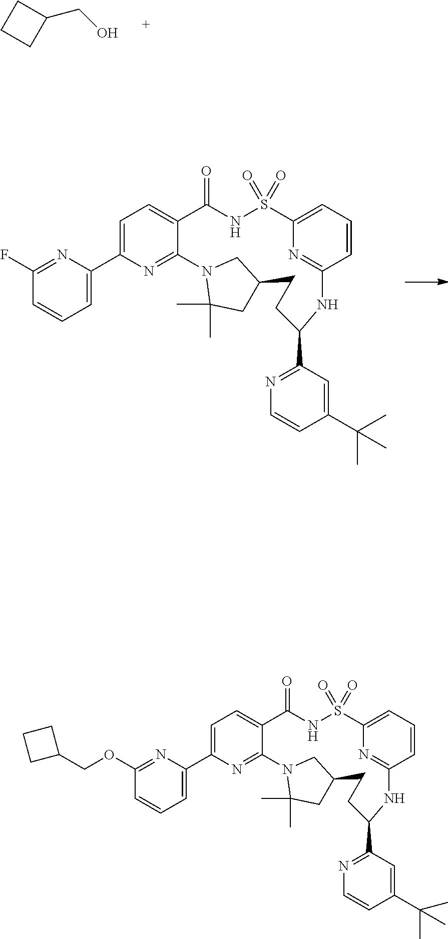 [SMILES: OCC1CCC1 ; CC(C)(C)c1ccnc([C@H]2CC[C@@H]3CN(c4nc(-c5cccc(F)n5)ccc4C(=O)NS(=O)(=O)c4cccc(n4)N2)C(C)(C)C3)c1 ; CC(C)(C)c1ccnc([C@H]2CC[C@@H]3CN(c4nc(-c5cccc(OCC6CCC6)n5)ccc4C(=O)NS(=O)(=O)c4cccc(n4)N2)C(C)(C)C3)c1]

To a solution of cyclobutylmethanol (174 μL of 0.25 M, 0.043 mmol) in DMF was added [bis(trimethylsilyl)amino]sodium (44 μL of 1 M, 0.044 mmol) and after stirring at rt for 1 min, it was added to (14S,17R)-17-(4-tert-butylpyridin-2-yl)-8-(6-fluoropyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (5.6 mg, 0.0087 mmol). Then the mixture was stirred at 80° C. for 200 min, diluted with 5 mL DCM and then 0.2 mL of 1 N HCl and 5 mL water was added (pH paper <4). The mixture was partitioned, the aqueous layer extracted with DCM, the organics dried (MgSO₄) and evaporated. The residue was subjected to preparative SFC eluting a gradient of 5 mM NH₃ in methanol to CO₂ (15-50% over 10 min) though a 21.2×250 mm 2-PIC column, 5 μm particle, giving as a white solid the target: (14S,17R)-17-(4-tert-butylpyridin-2-yl)-8-[6-(cyclobutylmethoxy)pyridin-2-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (2.8 mg, 45%). ¹H NMR (400 MHz, Chloroform-d) δ 8.48 (d, J=5.3 Hz, 1H), 8.17 (d, J=8.1 Hz, 1H), 7.99 (d, J=8.1 Hz, 1H), 7.86 (d, J=7.4 Hz, 1H), 7.68 (t, J=7.8 Hz, 1H), 7.58 (d, J=4.6 Hz, 2H), 7.21 (dd, J=5.4, 1.9 Hz, 1H), 6.78 (d, J 8.2 Hz, 1H), 6.70-6.63 (m, 1H), 6.02 (d, J=8.1 Hz, 1H), 5.31 (q, J=6.9 Hz, 1H), 4.39 (d, J=6.8 Hz, 2H), 3.54 (t, J=8.4 Hz, 1H), 3.21 (s, 1H), 3.02 (t, J=10.2 Hz, 1H), 2.82 (p, J=7.2 Hz, 1H), 2.33 (t, J=10.8 Hz, 1H), 2.15 (dq, J=9.8, 6.1 Hz, 2H), 1.94 (tq, J 16.3, 8.4 Hz, 5H), 1.75 (s, 3H), 1.72 (s, 3H), 1.68-1.43 (m, 5H), 1.33 (s, 9H). ESI-MS m/z calc. 709.341, found 710.5 (M+1)⁺; Retention time: 1.62 minutes (LC method A).

### Example 116: Preparation of (14S,17R)-17-(4-tert-butylpyridin-2-yl)-8-[6-(2-cyclopropylethoxy)pyridin-2-yl]-12,12-dimethyl-2λ⁶-thia-3,9,11,18,23-pentaazatetracyclo [17.3.1.111,14.05,10]tetracosa-1(22),5,7,9,19(23),20-hexaene-2,2,4-trione (Compound 301)

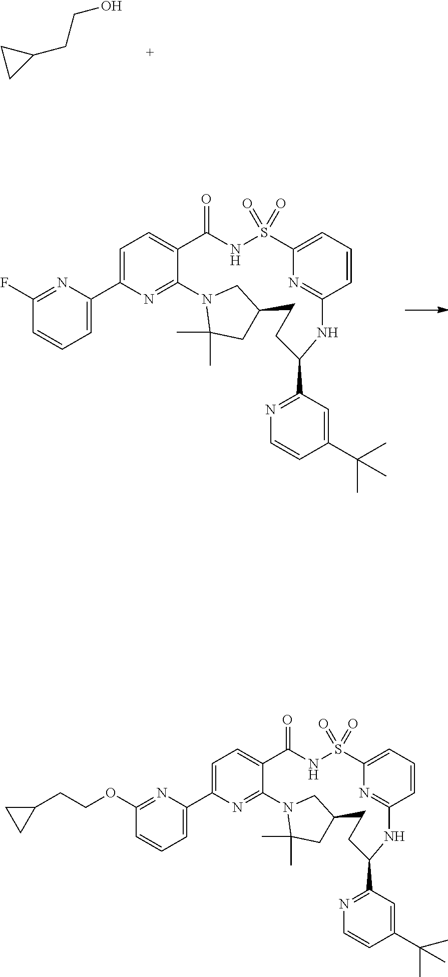 [SMILES: OCCC1CC1 ; CC(C)(C)c1ccnc([C@H]2CC[C@@H]3CN(c4nc(-c5cccc(F)n5)ccc4C(=O)NS(=O)(=O)c4cccc(n4)N2)C(C)(C)C3)c1 ; CC(C)(C)c1ccnc([C@H]2CC[C@@H]3CN(c4nc(-c5cccc(OCCC6CC6)n5)ccc4C(=O)NS(=O)(=O)c4cccc(n4)N2)C(C)(C)C3)c1]

To a solution of 2-cyclopropylethanol (approximately 174.0 μL of 0.25 M, 0.043 mmol) in DMF was added [bis(trimethylsilyl)amino]sodium (approximately 43.50 μL of 1 M in THF, 0.043 mmol) and after stirring at rt for 1 min, it was added to (14S,17R)-17-(4-tert-butylpyridin-2-yl)-8-(6-fluoropyridin-2-yl)-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (5.6 mg, 0.008699 mmol). Then the mixture was stirred at 80° C. for 200 min, diluted with 5 mL DCM and then 0.2 mL of 1 N HCl and 5 mL water was added (pH paper <4). The mixture was partitioned, the aqueous layer extracted with DCM, the organics dried (MgSO₄) and evaporated. The residue was subjected to preparative SFC eluting a gradient of 5 mM NH₃ in methanol to CO₂ (15-50% over 10 min) though a 21.2×250 mm 2-PIC column, 5 μm particle, giving as a white solid the target: (14S,17R)-17-(4-tert-butylpyridin-2-yl)-8-[6-(2-cyclopropylethoxy)pyridin-2-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (2.3 mg, 37%). ¹H NMR (400 MHz, Chloroform-d) δ 8.48 (d, J=5.2 Hz, 1H), 8.16 (d, J=8.0 Hz, 1H), 7.98 (d, J=8.1 Hz, 1H), 7.87 (d, J=7.4 Hz, 1H), 7.68 (t, J=7.9 Hz, 1H), 7.57 (d, J=4.7 Hz, 2H), 7.21 (d, J=5.4 Hz, 1H), 6.77 (d, J=8.2 Hz, 1H), 6.66 (d, J=6.5 Hz, 1H), 6.01 (s, 1H), 5.31 (d, J=7.2 Hz, 1H), 4.49 (t, J=6.8 Hz, 2H), 3.53 (d, J=8.6 Hz, 1H), 3.21 (s, 1H), 3.07-2.99 (m, 1H), 2.31 (d, J=10.9 Hz, 1H), 1.94 (dd, J=14.6, 7.4 Hz, 1H), 1.75 (s, 3H), 1.71 (s, 3H), 1.69-1.41 (m, 5H), 1.33 (s, 9H), 1.26 (s, 2H), 0.93-0.80 (m, 1H), 0.50 (d, J=7.7 Hz, 2H), 0.14 (d, J=5.0 Hz, 2H). ESI-MS m/z calc. 709.341, found 710.5 (M+1)⁺; Retention time: 1.59 minutes (LC method A).

### Example 117: Preparation of (14S,17R)-8-bromo-12,12-dimethyl-17-[6-(trifluoromethyl)pyridin-2-yl]-2λ⁶-thia-3,9,11,18,23-pentaazatetracyclo [17.3.1.111,14.05,10]tetracosa-1(22),5,7,9,19(23),20-hexaene-2,2,4-trione, and (14S,17S)-8-bromo-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione

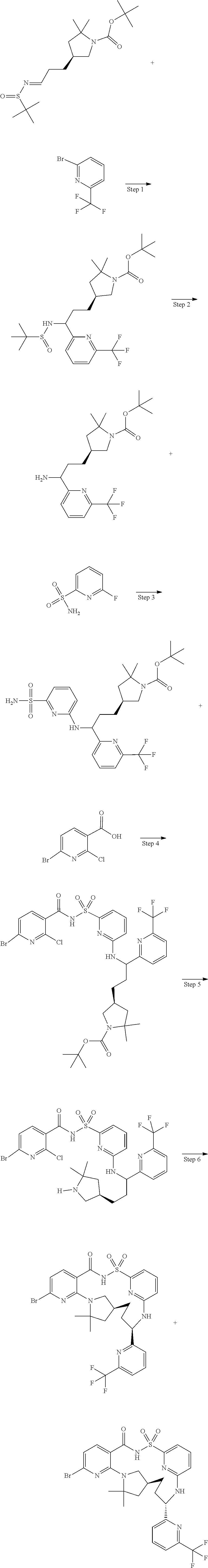

### Step 1: tert-Butyl (4S)-4-[3-(tert-butylsulfinylamino)-3-[6-(trifluoromethyl)-2-pyridyl]propyl]-2,2-dimethyl-pyrrolidine-1-carboxylate

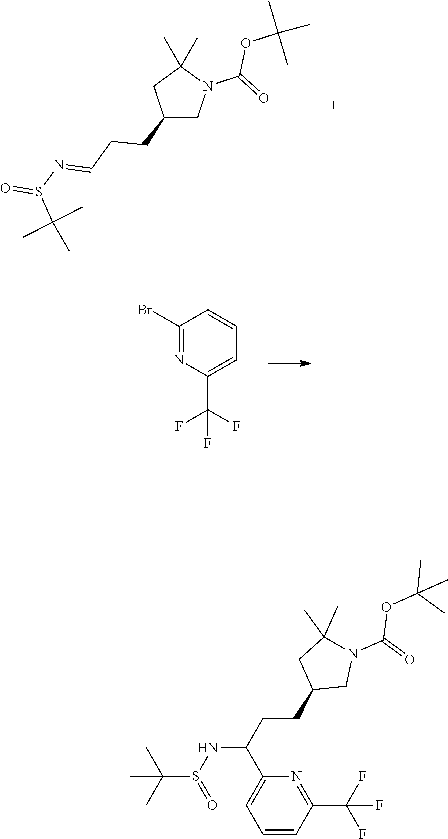

2-Bromo-6-(trifluoromethyl)pyridine (6.6 g, 28.62 mmol) was dissolved in diethyl ether (100 mL). The solution was cooled in a dry ice acetone bath and stirred under nitrogen balloon for 15 min. n-BuLi (11.5 mL of 2.5 M in hexanes, 28.75 mmol) was added within 1 min. The light yellowish solution was stirred at <−70° C. for 45 min. tert-Butyl (4S)-4-[(3E)-3-tert-butylsulfinyliminopropyl]-2,2-dimethyl-pyrrolidine-1-carboxylate (5.4 g, 14.31 mmol) was then added as a solution of THF (8 mL plus 2 mL rinse). The mixture was further stirred at the same temperature for 45 min. NH₄Cl (40 mL, saturated aqueous) was added, followed by water (50 mL) and EtOAc (100 mL). The mixture was allowed to warm up to rt and combined with another batch of crude material prepared in the same conditions on 360 mg scale. The layers were separated, and the organic layer was washed with brine, dried over anhydrous MgSO₄, filtered and concentrated. The residue was purified by silica gel chromatography (80 g column), using 10-80% EtOAc in hexanes to afford tert-butyl (4S)-4-[3-(tert-butylsulfinylamino)-3-[6-(trifluoromethyl)-2-pyridyl]propyl]-2,2-dimethyl-pyrrolidine-1-carboxylate (6.96 g, 91% adjusted yield) as a glassy solid. ESI-MS m/z calc. 505.2586, found 506.6 (M+1)⁺; Retention time: 3.79 minutes (LC method B).

### Step 2: tert-Butyl (4S)-4-[3-amino-3-[6-(trifluoromethyl)-2-pyridyl]propyl]-2,2-dimethyl-pyrrolidine-1-carboxylate

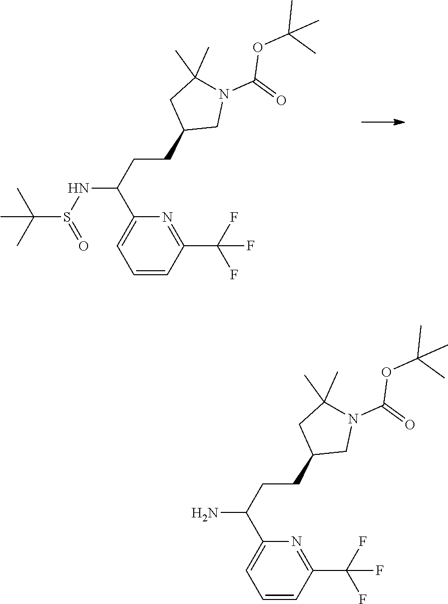 [SMILES: CC(C)(C)OC(=O)N1C[C@@H](CCC(NS(=O)C(C)(C)C)c2cccc(C(F)(F)F)n2)CC1(C)C ; CC(C)(C)OC(=O)N1C[C@@H](CCC(N)c2cccc(C(F)(F)F)n2)CC1(C)C]

tert-Butyl (4S)-4-[3-(tert-butylsulfinylamino)-3-[6-(trifluoromethyl)-2-pyridyl]propyl]-2,2-dimethyl-pyrrolidine-1-carboxylate (7.45 g, 13.997 mmol) was dissolved in a mixture of THF (100 mL) and Water (20 mL). Molecular iodine (1.05 g, 4.14 mmol) was added. The mixture was then heated in a 50° C. oil bath and stirred 3h. It was cooled to rt, treated with Na₂S₂O₃ (5 g in 50 mL of saturated aqueous NaHCO₃) and diluted with EtOAc (100 mL). The layers were separated, and the organic layer was washed with brine, dried over anhydrous MgSO₄, filtered and concentrated. The residue was purified by silica gel chromatography (80 g column), using 0-10% MeOH in DCM to afford tert-butyl (4S)-4-[3-amino-3-[6-(trifluoromethyl)-2-pyridyl]propyl]-2,2-dimethyl-pyrrolidine-1-carboxylate as a light brown foam. (4.6 g, 78%). ESI-MS m/z calc. 401.229, found 402.6 (M+1)⁺; Retention time: 2.86 minutes. (LC method B).

### Step 3: tert-Butyl (4S)-2,2-dimethyl-4-[3-[(6-sulfamoyl-2-pyridyl)amino]-3-[6-(trifluoromethyl)-2-pyridyl]propyl]pyrrolidine-1-carboxylate

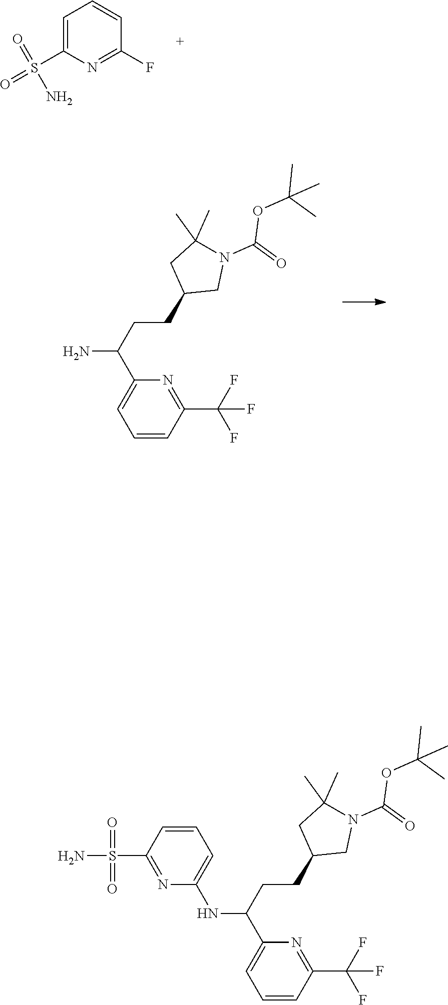 [SMILES: NS(=O)(=O)c1cccc(F)n1 ; CC(C)(C)OC(=O)N1C[C@@H](CCC(N)c2cccc(C(F)(F)F)n2)CC1(C)C ; CC(C)(C)OC(=O)N1C[C@@H](CCC(Nc2cccc(S(N)(=O)=O)n2)c2cccc(C(F)(F)F)n2)CC1(C)C]

tert-Butyl (4S)-4-[3-amino-3-[6-(trifluoromethyl)-2-pyridyl]propyl]-2,2-dimethyl-pyrrolidine-1-carboxylate (4.6 g, 10.88 mmol) was dissolved in DMSO (6 mL). 6-Fluoropyridine-2-sulfonamide (1.92 g, 10.90 mmol) was added in one portion, followed by Na₂CO₃ (3.5 g, 33.02 mmol). The mixture was placed in a pre-heated 110° C. oil bath and stirred under a nitrogen balloon for 20 h. It was then cooled to rt, diluted with water (30 mL) and EtOAc (50 mL). The layers were separated and the organic layer was washed with more water (30 mL), brine (30 mL), dried over anhydrous MgSO₄, filtered and concentrated. The residue was purified by silica gel chromatography (120 g column), using 5-50% EtOAc in hexanes to afford tert-butyl (4S)-2,2-dimethyl-4-[3-[(6-sulfamoyl-2-pyridyl)amino]-3-[6-(trifluoromethyl)-2-pyridyl]propyl]pyrrolidine-1-carboxylate as a light brownish foam (3.7738 g, 60%). ¹H NMR (500 MHz, DMSO-d₆) δ 8.01 (t, J=7.8 Hz, 1H), 7.87 (td, J=9.3, 8.4, 2.0 Hz, 1H), 7.74 (d, J=7.6 Hz, 1H), 7.64 (d, J=8.4 Hz, 1H), 7.57-7.50 (m, 1H), 7.07 (s, 2H), 6.98 (d, J=7.2 Hz, 1H), 6.73 (d, J=8.2 Hz, 1H), 5.26 (s, 1H), 3.52 (dt, J=12.0, 6.7 Hz, 1H), 2.76 (dtd, J=25.7, 10.6, 5.3 Hz, 1H), 2.16-2.02 (m, 1H), 1.98-1.78 (m, 3H), 1.49-1.28 (m, 15H), 1.22 (s, 3H). ESI-MS m/z calc. 557.2284, found 558.4 (M+1)⁺; Retention time: 2.84 minutes (LC method H).

### Step 4: tert-Butyl (4S)-4-[3-[[6-[(6-bromo-2-chloro-pyridine-3-carbonyl)sulfamoyl]-2-pyridyl]amino]-3-[6-(trifluoromethyl)-2-pyridyl]propyl]-2,2-dimethyl-pyrrolidine-1-carboxylate

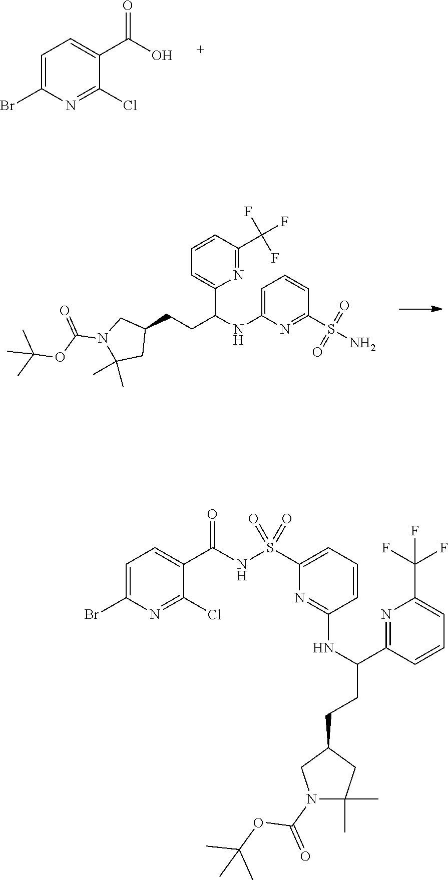 [SMILES: O=C(O)c1ccc(Br)nc1Cl ; CC(C)(C)OC(=O)N1C[C@@H](CCC(Nc2cccc(S(N)(=O)=O)n2)c2cccc(C(F)(F)F)n2)CC1(C)C ; CC(C)(C)OC(=O)N1C[C@@H](CCC(Nc2cccc(S(=O)(=O)NC(=O)c3ccc(Br)nc3Cl)n2)c2cccc(C(F)(F)F)n2)CC1(C)C]

A mixture of 6-bromo-2-chloro-pyridine-3-carboxylic acid (510 mg, 2.16 mmol) and CDI (350 mg, 2.16 mmol) were dissolved in THF (3.6 mL) and the mixture stirred at 60° C. for 45 min then tert-butyl (4S)-2,2-dimethyl-4-[3-[(6-sulfamoyl-2-pyridyl)amino]-3-[6-(trifluoromethyl)-2-pyridyl]propyl]pyrrolidine-1-carboxylate (600 mg, 1.076 mmol) was added followed by DBU (726 μL, 4.85 mmol) and the resulting mixture was stirred at rt for 4 h. The mixture was diluted with EtOAc and washed with 1 N HCl in water, water, brine, dried (MgSO₄), and concentrated. The residue was purified (flash chromatography: 40 g SiO2, 10-35% EtOAc in hexanes) to provide the desired product: tert-butyl (4S)-4-[3-[[6-[(6-bromo-2-chloro-pyridine-3-carbonyl)sulfamoyl]-2-pyridyl]amino]-3-[6-(trifluoromethyl)-2-pyridyl]propyl]-2,2-dimethyl-pyrrolidine-1-carboxylate (590 mg, 71%). ¹H NMR (400 MHz, Chloroform-d) δ 7.89 (dq, J=13.7, 7.5, 6.8 Hz, 1H), 7.72 (dd, J=13.6, 8.0 Hz, 1H), 7.63 (d, J=7.8 Hz, 1H), 7.60-7.51 (m, 2H), 7.45 (ddd, J=17.1, 10.3, 6.5 Hz, 2H), 6.74-6.65 (m, 1H), 5.82 (d, J=9.4 Hz, 1H), 5.35-5.16 (m, 1H), 4.50-4.17 (m, 1H), 4.16-4.08 (m, 1H), 3.11 (q, J=10.2 Hz, 1H), 2.4-2.3 (m, 1H), 2.2-2.1 (m, 1H), 2.1-1.9 (m, 1H), 1.8-1.65 (m, 2H), 1.6-1.5 (m, 2H), 1.40-1.35 (m, 9H), 1.35-1.25 (m, 6H). ESI-MS m/z calc. 776.1193, found 776.9 (M+1)⁺; Retention time: 0.8 minutes (LC method D).

### Step 5: 6-Bromo-2-chloro-N-[[6-[[3-[(3R)-5,5-dimethylpyrrolidin-3-yl]-1-[6-(trifluoromethyl)-2-pyridyl]propyl]amino]-2-pyridyl]sulfonyl]pyridine-3-carboxamide

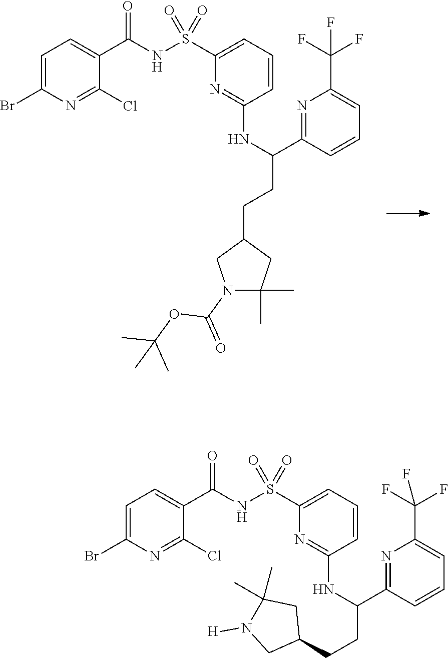 [SMILES: CC(C)(C)OC(=O)N1CC(CCC(Nc2cccc(S(=O)(=O)NC(=O)c3ccc(Br)nc3Cl)n2)c2cccc(C(F)(F)F)n2)CC1(C)C ; CC1(C)C[C@@H](CCC(Nc2cccc(S(=O)(=O)NC(=O)c3ccc(Br)nc3Cl)n2)c2cccc(C(F)(F)F)n2)CN1]

To a solution of tert-butyl (4S)-4-[3-[[6-[(6-bromo-2-chloro-pyridine-3-carbonyl)sulfamoyl]-2-pyridyl]amino]-3-[6-(trifluoromethyl)-2-pyridyl]propyl]-2,2-dimethyl-pyrrolidine-1-carboxylate (590 mg, 0.7602 mmol) in DCM (4 mL) was added TFA (1.8 mL, 23.36 mmol) and the mixture was stirred at rt for 1 h. The solution was concentrated to dryness under reduced pressure, then co-evaporated with THF (2×20 mL), then dried under high vacuum at rt/16h to provide: 6-bromo-2-chloro-N-[[6-[[3-[(3R)-5,5-dimethylpyrrolidin-3-yl]-1-[6-(trifluoromethyl)-2-pyridyl]propyl]amino]-2-pyridyl]sulfonyl]pyridine-3-carboxamide (trifluoroacetate salt) (772 mg, 100%). ESI-MS m/z calc. 674.0689, found 676.9 (M+1)⁺; Retention time: 0.5 minutes (LC method D).

### Step 6: (14S,17R)-8-Bromo-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione, and (14S,17S)-8-bromo-12,12-dimethyl-17-[6-(trifluoromethyl) pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione

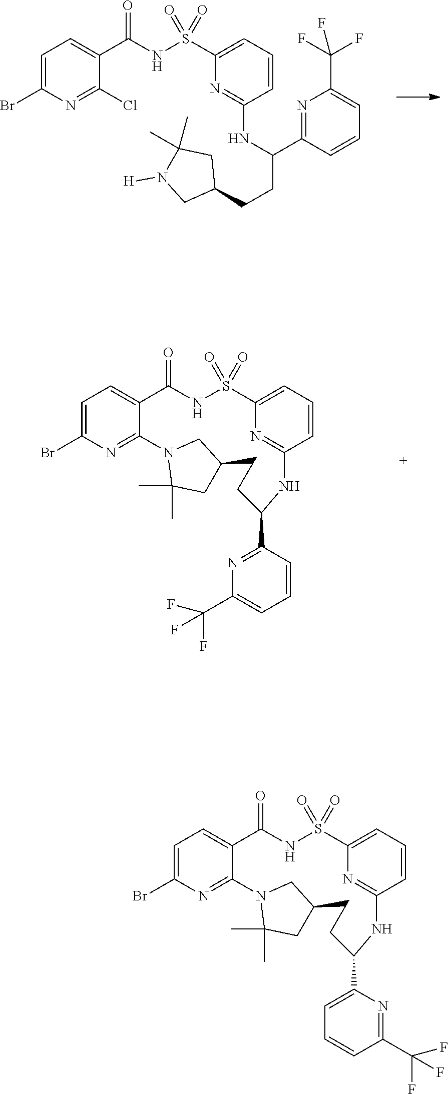 [SMILES: CC1(C)C[C@@H](CCC(Nc2cccc(S(=O)(=O)NC(=O)c3ccc(Br)nc3Cl)n2)c2cccc(C(F)(F)F)n2)CN1 ; CC1(C)C[C@@H]2CC[C@H](c3cccc(C(F)(F)F)n3)Nc3cccc(n3)S(=O)(=O)NC(=O)c3ccc(Br)nc3N1C2 ; CC1(C)C[C@@H]2CC[C@@H](c3cccc(C(F)(F)F)n3)Nc3cccc(n3)S(=O)(=O)NC(=O)c3ccc(Br)nc3N1C2]

A mixture 6-bromo-2-chloro-N-[[6-[[3-[(3R)-5,5-dimethylpyrrolidin-3-yl]-1-[6-(trifluoromethyl)-2-pyridyl]propyl]amino]-2-pyridyl]sulfonyl]pyridine-3-carboxamide (trifluoroacetate salt) (772 mg, 0.7583 mmol) and potassium carbonate (629 mg, 4.551 mmol) in DMA (7.7 mL) was heated at 140° C. for 7 h, cooled by ice bath, diluted with 100 mL of EtOAc and 30 mL of water and then 15 mL of 1 M HCl which produced a mixture with a pH=2 by paper. The mixture was partitioned and the organic layer separated. The aqueous layer was extracted with 100 mL of EtOAc. The combined organic extracts were dried (MgSO₄) and evaporated to an oil. The residue was dissolved into DMSO (100 mg/mL) and diluted with 1 volumes of DMA. This solution was purified with preparative SFC eluting a gradient of 5 mM NH₃ in methanol to CO₂ (40-70% over 10 min) through a 21.2×250 mm 2-PIC column, 5 μm particle giving as white solids after evaporation of the collected fractions:

First diastereomer to elute: (14S,17R)-8-bromo-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione (101 mg, 21%). ¹H NMR (400 MHz, Chloroform-d) δ 7.80 (t, J=7.9 Hz, 1H), 7.63 (d, J=8.1 Hz, 1H), 7.57 (t, J=9.0 Hz, 1H), 7.52 (d, J=7.9 Hz, 1H), 7.46 (d, J=7.3 Hz, 1H), 6.77 (d, J=7.9 Hz, 1H), 6.71 (d, J=8.4 Hz, 1H), 5.54-5.41 (m, 2H), 3.33 (d, J 9.6 Hz, 1H), 2.97 (d, J=10.6 Hz, 1H), 2.54 (s, 2H), 1.96 (p, J=6.0 Hz, 3H), 1.68 (s, 2H), 1.63 (s, 3H), 1.58 (s, 2H), 1.55 (s, 3H). ESI-MS m/z calc. 638.0923, found 639.2 (M+1)⁺; Retention time: 1.79 minutes (LC method A).

Second diastereomer to elute: (14S,17S)-8-bromo-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione (77 mg, 16%). ¹H NMR (400 MHz, Chloroform-d) δ 7.90 (t, J=7.8 Hz, 1H), 7.85 (d, J=8.1 Hz, 1H), 7.67-7.58 (m, 3H), 7.54 (d, J=8.0 Hz, 1H), 6.99 (d, J=7.9 Hz, 1H), 6.79 (d, J=7.4 Hz, 1H), 6.47 (s, 1H), 5.01 (s, 1H), 3.55 (t, J=8.6 Hz, 1H), 3.23 (s, 1H), 2.34 (d, J=12.9 Hz, 1H), 2.02-1.80 (m, 5H), 1.72 (t, J=11.5 Hz, 1H), 1.60 (s, 3H), 1.53 (s, 3H). ESI-MS m/z calc. 638.0923, found 639.3 (M+1)⁺; Retention time: 1.86 minutes (LC method A).

### Example 118: Preparation of (14S,17R)-8-[3-(3,3-dimethylbutoxy)-1H-pyrazol-1-yl]-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione (Compound 306)

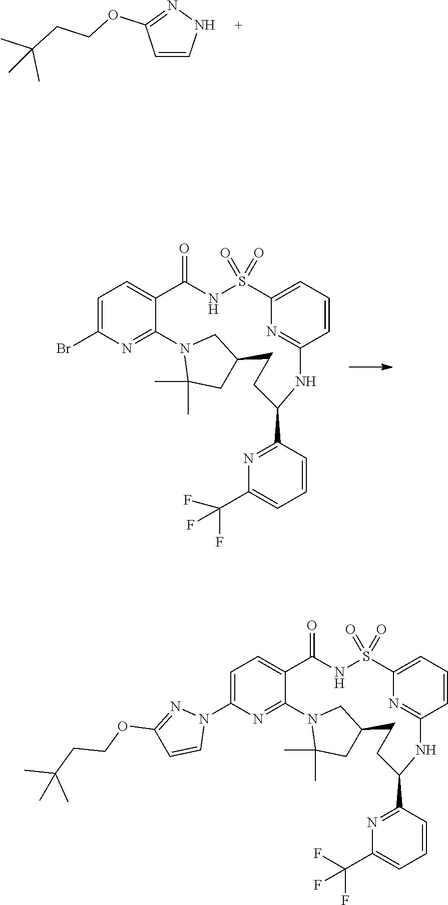 [SMILES: CC(C)(C)CCOc1cc[nH]n1 ; CC1(C)C[C@@H]2CC[C@H](c3cccc(C(F)(F)F)n3)Nc3cccc(n3)S(=O)(=O)NC(=O)c3ccc(Br)nc3N1C2 ; CC(C)(C)CCOc1ccn(-c2ccc3c(n2)N2C[C@@H](CC[C@H](c4cccc(C(F)(F)F)n4)Nc4cccc(n4)S(=O)(=O)NC3=O)CC2(C)C)n1]

A mixture of (14S,17R)-8-bromo-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione (45 mg, 0.07037 mmol), 3-(3,3-dimethylbutoxy)-1H-pyrazole (47 mg, 0.28 mmol), hexaphenyl-1,7-dipalladanonacyclo [5.5.5.01,4.01,11.01,14.02,4.05,7.07,9.07,16] heptadeca-2,5,8,11,13,16-hexaene-4,10,15-trione (3.1 mg, 0.0034 mmol), (5-diphenylphosphanyl-9,9-dimethyl-xanthen-4-yl)-diphenyl-phosphane (6.2 mg, 0.011 mmol), Cs₂CO₃ (92 mg, 0.28 mmol) and DMF (700 pt) was degassed by bubbling nitrogen for 1 min then heated to 100° C. for 16 h. Then the mixture was diluted with 2 mL water, 2 mL of DCM, then 0.6 mL of 1 M HCl added and the mixture partitioned, when paper showed pH=4. The layers were separated and the aqueous layer extracted with DCM (2 mL). The combined organic extracts were dried (MgSO₄) and evaporated. The residue was dissolved in 1:1 MeOH/ACN at 40 mg/mL and subjected to preparative SFC eluting a gradient of 5 mM NH₃ in methanol to CO₂ (15-50% over 10 min) though a 21.2×250 mm 2-PIC column, 5 μm particle, giving as a white solid: (14S,17R)-8-[3-(3,3-dimethylbutoxy)-1H-pyrazol-1-yl]-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione (6.0 mg, 12%). ¹H NMR (400 MHz, Chloroform-d) δ 8.19 (d, J=2.8 Hz, 1H), 8.02 (d, J=8.3 Hz, 1H), 7.83 (t, J=7.9 Hz, 1H), 7.64-7.56 (m, 2H), 7.54 (d, J=7.5 Hz, 2H), 7.24 (s, 1H), 6.71 (d, J=8.3 Hz, 1H), 5.91 (d, J=2.8 Hz, 1H), 5.69 (d, J=8.9 Hz, 1H), 5.49 (d, J=7.6 Hz, 1H), 4.32 (t, J 7.4 Hz, 2H), 3.44 (s, 1H), 3.01 (t, J=10.3 Hz, 1H), 2.86 (d, J=14.3 Hz, 1H), 2.14 (t, J 10.4 Hz, 1H), 1.96 (s, 1H), 1.87 (s, 1H), 1.74 (t, J=7.4 Hz, 2H), 1.66 (s, 3H), 1.64 (s, 3H), 1.59 (d, J=12.4 Hz, 3H), 1.00 (s, 9H). ESI-MS m/z calc. 726.29236, found 727.6 (M+1)⁺; Retention time: 2.15 minutes (LC method A).

### Example 119: Preparation of (14S,17R)-8-(6-fluoropyridin-2-yl)-12,12-dimethyl-17-[6-(trifluoromethyl)pyridin-2-yl]-2λ⁶-thia-3,9,11,18,23-pentaazatetracyclo [17.3.1.111,14.05,10]tetracosa-1(22),5,7,9,19(23),20-hexaene-2,2,4-trione (Compound 308)

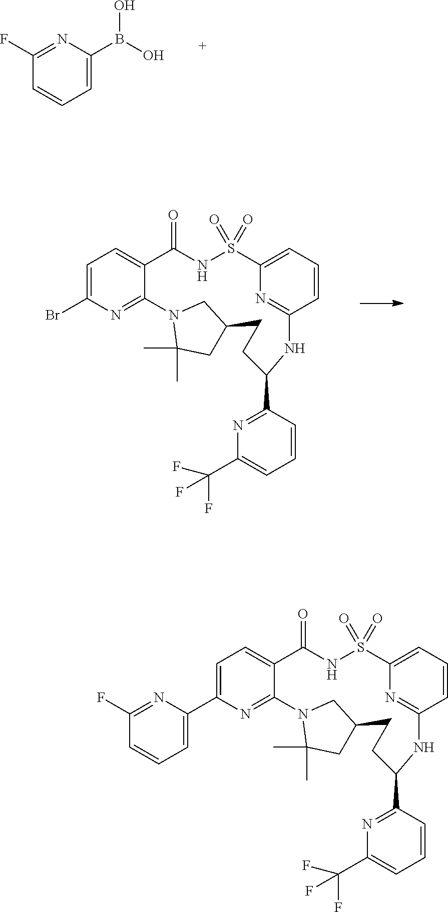 [SMILES: OB(O)c1cccc(F)n1 ; CC1(C)C[C@@H]2CC[C@H](c3cccc(C(F)(F)F)n3)Nc3cccc(n3)S(=O)(=O)NC(=O)c3ccc(Br)nc3N1C2 ; CC1(C)C[C@@H]2CC[C@H](c3cccc(C(F)(F)F)n3)Nc3cccc(n3)S(=O)(=O)NC(=O)c3ccc(-c4cccc(F)n4)nc3N1C2]

A mixture of (14S,17R)-8-bromo-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione (48 mg, 0.075 mmol), (6-fluoro-2-pyridyl)boronic acid (32 mg, 0.23 mmol), Pd(dppf)Cl₂ (5.5 mg, 0.0075 mmol), acetonitrile (840 μL) and NaHCO₃(446 μL of 1 M, 0.446 mmol) was degassed by bubbling nitrogen for 1 min then heated to 100° C. for 2 h. Then the mixture was diluted with 2 mL water, 2 mL of 5:1 DCM/MeOH, then 0.5 mL of 1 M HCl added and the mixture partitioned, when paper showed pH 4. The layers were separated and the aqueous layer extracted with 5:1 DCM/MeOH (2 mL). The residue was purified by flash chromatography (4 g SiO₂, 10-40% EtOAc in hexanes over 15 min) to provide the target: (14S,17R)-8-(6-fluoropyridin-2-yl)-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione (47.8 mg, 97%). ¹H NMR (400 MHz, Chloroform-d) δ 9.95 (s, 1H), 8.17 (dd, J 7.3, 2.1 Hz, 1H), 8.09 (d, J=8.0 Hz, 1H), 7.89 (tt, J=17.0, 7.9 Hz, 3H), 7.58 (ddd, J 24.3, 13.5, 7.8 Hz, 4H), 6.97 (dd, J=8.0, 2.7 Hz, 1H), 6.73 (d, J=8.2 Hz, 1H), 5.73 (d, J=8.8 Hz, 1H), 5.51 (q, J=7.7 Hz, 1H), 3.96 (q, J=7.1, 6.4 Hz, 1H), 3.78-3.71 (m, 1H), 3.45 (dd, J=9.6, 6.7 Hz, 1H), 3.04 (t, J=10.2 Hz, 1H), 2.92 (t, J=14.5 Hz, 1H), 2.20 (dd, J=12.4, 8.2 Hz, 1H), 1.98 (d, J=7.3 Hz, 1H), 1.91-1.79 (m, 2H), 1.73 (s, 3H), 1.72 (s, 3H), 1.65 (d, J=11.0 Hz, 1H). ESI-MS m/z calc. 655.19885, found 656.5 (M+1)⁺; Retention time: 1.78 minutes (LC method A).

### Example 120: Preparation of (14S,17R)-8-[6-(3,3-dimethylbutoxy)pyridin-2-yl]-12,12-dimethyl-17-[6-(trifluoromethyl)pyridin-2-yl]-2λ⁶-thia-3,9,11,18,23-pentaazatetracyclo[17.3.1.111,14.05,10]tetracosa-1 (22),5,7,9,19 (23),20-hexaene-2,2,4-trione (Compound 304)

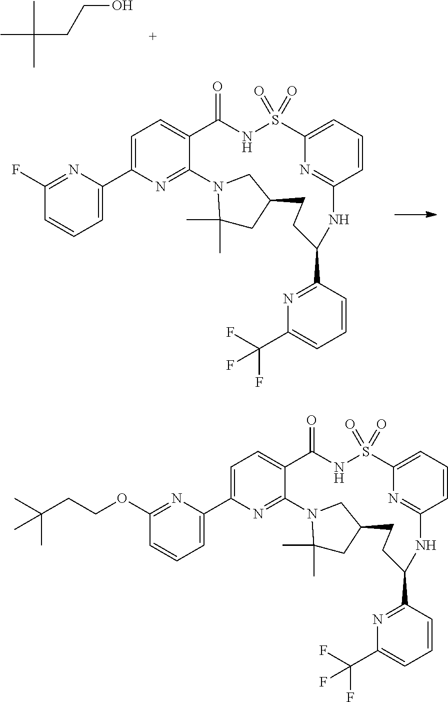

To a solution of 3,3-dimethylbutan-1-ol (610 μL, of 0.25 M, 0.152 mmol) in DMF was added [bis(trimethylsilyl)amino]sodium (152 μL, of 1 M, 0.152 mmol) and after stirring at rt for 1 min, then this solution was added (14S,17R)-8-(6-fluoropyridin-2-yl)-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione (20 mg, 0.0305 mmol). Then the mixture was stirred at 80° C. for 1 h, diluted with 5 mL DCM and then 0.2 mL of 1 N HCl and 5 mL water was added (pH paper <4). The mixture was partitioned, the aqueous layer extracted with DCM, the organics dried (MgSO₄) and evaporated. The residue was subjected to preparative SFC eluting a gradient of 5 mM NH₃ in methanol to CO₂ (15-50% over 10 min) through a 21.2×250 mm 2-PIC column, 5 μm particle, giving as a white solid the target: (14S,17R)-8-[6-(3,3-dimethylbutoxy)pyridin-2-yl]-12,12-dimethyl-17-[6-(trifluoromethyl)pyridin-2-yl]-2λ⁶-thia-3,9,11,18,23-pentaazatetracyclo[17.3.1.111,14.05,10]tetracosa-1(22),5,7,9,19(23),20-hexaene-2,2,4-trione (4.3 mg, 19%). ¹H NMR (400 MHz, Chloroform-d) δ 8.11 (s, 1H), 7.90 (s, 1H), 7.85 (d, J=7.3 Hz, 2H), 7.66 (t, J=7.9 Hz, 1H), 7.58 (s, 4H), 6.73 (t, J 8.0 Hz, 2H), 5.83 (s, 1H), 5.50 (s, 1H), 4.49 (t, J=7.4 Hz, 2H), 3.46 (s, 1H), 3.02 (d, J 7.6 Hz, 2H), 2.23 (s, 1H), 1.97 (s, 1H), 1.76 (d, J=7.6 Hz, 2H), 1.71 (s, 2H), 1.66-1.52 (m, 8H), 1.02 (s, 9H). ESI-MS m/z calc.

### Example 121: Preparation of (14S)-12,12-Dimethyl-17-(propan-2-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (diastereomer 1, Compound 358) and (14S)-12,12-dimethyl-17-(propan-2-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (diastereomer 2, Compound 357)

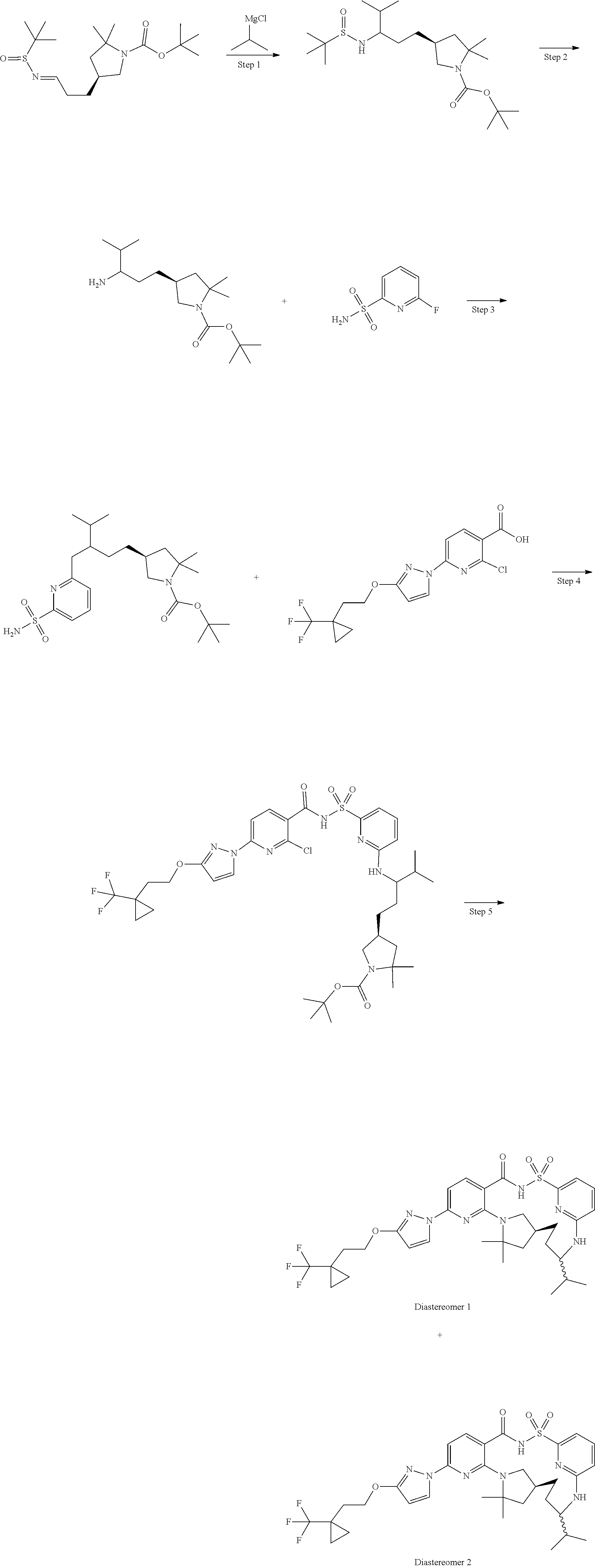

### Step 1: tert-Butyl (4S)-4-[3-(tert-butylsulfinylamino)-4-methyl-pentyl]-2,2-dimethyl-pyrrolidine-1-carboxylate

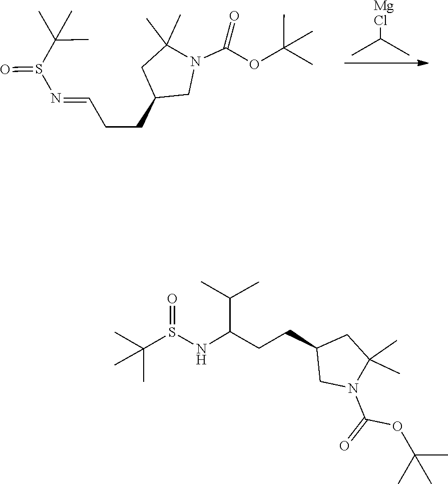

tert-Butyl (4S)-4-[(3E)-3-tert-butylsulfinyliminopropyl]-2,2-dimethyl-pyrrolidine-1-carboxylate (10 g, 26.496 mmol) was dissolved in THF (150 mL). The solution was cooled to −78° C. using a dry ice acetone bath under nitrogen balloon and stirred 5 min. Isopropyl magnesium chloride (125 mL of 1.3 M in THF, 162.50 mmol) was added dropwise. The mixture was let warm up to ˜−35° C. and stirred 1 hour. Saturated aqueous NH₄Cl (80 mL) was added. The mixture was warmed up to rt. EtOAc (200 mL) and water (100 mL) was added. The layers were separated and the aqueous layer was extracted one more time with EtOAc (200 mL). The combined organics was washed with brine, dried over anhydrous Na₂SO₄, filtered and concentrated to give crude tert-butyl(4S)-4-[3-(tert-butylsulfinylamino)-4-methyl-pentyl]-2,2-dimethyl-pyrrolidine-1-carboxylate (12.5 g, 100%). ESI-MS m/z calc. 402.2916, found 403.6 (M+1)⁺; Retention time: 4.02 minutes (LC method B).

### Step 2: tert-Butyl (4S)-4-(3-amino-4-methyl-pentyl)-2,2-dimethyl-pyrrolidine-1-carboxylate

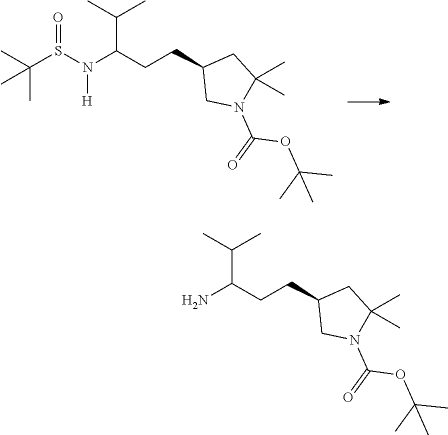

tert-Butyl (4S)-4-[3-(tert-butylsulfinylamino)-4-methyl-pentyl]-2,2-dimethyl-pyrrolidine-1-carboxylate (12.5 g, 26.39 mmol) was dissolved in a solvent mixture of THF (180 mL) and water (36 mL). Molecular iodine (2 g, 7.86 mmol) was added in one portion. The mixture was stirred at 35° C. for 18h. It was then cooled to rt and partitioned between EtOAc (300 mL) and Na₂S₂O₃ (50 g) in saturated aqueous NaHCO₃(300 mL). The layers were separated and the aqueous layer was extracted one more time with EtOAc (200 mL). The organic layer was concentrated. The residue was dissolved in 1M HCl (500 mL) and was extracted with diethyl ether (300 mL). The aqueous layer was basified by 2.5M NaOH and extracted with EtOAc (2×300 mL). The organic layer was washed with brine, dried over anhydrous Na₂SO₄, filtered and concentrated to give tert-butyl (4S)-4-(3-amino-4-methyl-pentyl)-2,2-dimethyl-pyrrolidine-1-carboxylate (6.8 g, 82%). ESI-MS m/z calc. 298.262, found 299.7 (M+1)⁺; Retention time: 2.69 minutes (LC method B).

### Step 3: tert-Butyl (4S)-2,2-dimethyl-4-[4-methyl-3-[(6-sulfamoyl-2-pyridyl)amino]pentyl]pyrrolidine-1-carboxylate

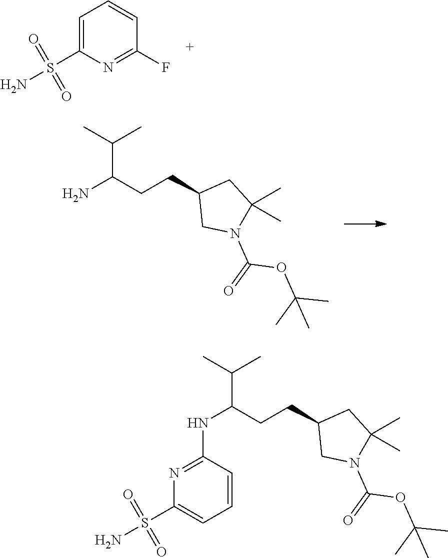

To a mixture of tert-butyl (4S)-4-(3-amino-4-methyl-pentyl)-2,2-dimethyl-pyrrolidine-1-carboxylate (6.8 g, 21.64 mmol) and 6-fluoropyridine-2-sulfonamide (7.5 g, 40.44 mmol) in DMSO (20 mL) was added DIEA (12 mL, 68.89 mmol). The mixture was stirred at 115° C. for 20 hours. It was then cooled to rt and partitioned between EtOAc (300 mL) and saturated sodium bicarbonate (500 mL). The aqueous layer was extracted with more EtOAc (300 mL). The combined EtOAc solution was washed with brine (2×500 mL), dried over anhydrous Na₂SO₄, filtered and concentrated. The residue was purified by silica gel chromatography, using 0-70% EtOAc in hexanes to afford tert-butyl (4S)-2,2-dimethyl-4-[4-methyl-3-[(6-sulfamoyl-2-pyridyl)amino]pentyl]pyrrolidine-1-carboxylate (8.1 g, 79%) as a solid. ¹H NMR (500 MHz, DMSO-d₆) δ 7.48 (t, J=7.8 Hz, 1H), 7.03 (s, 2H), 6.90 (d, J=7.1 Hz, 1H), 6.72 (d, J=9.1 Hz, 1H), 6.65 (d, J=8.3 Hz, 1H), 3.52 (dt, J=18.4, 10.7 Hz, 1H), 2.82-2.69 (m, 1H), 2.07 (d, J=10.9 Hz, 1H), 1.85 (d, J=17.6 Hz, 1H), 1.82-1.72 (m, 1H), 1.50 (d, J=11.8 Hz, 1H), 1.43-1.28 (m, 16H), 1.23 (d, J=2.9 Hz, 3H), 0.88 (dd, J=9.1, 6.7 Hz, 6H). ESI-MS m/z calc. 454.2614, found 455.2 (M+1)⁺; Retention time: 2.77 minutes (LC method H).

### Step 4: tert-Butyl (4S)-4-[3-[[6-[[2-chloro-6-[3-[2-[1-(trifluoromethyl)cyclopropyl] ethoxy]pyrazol-1-yl]pyridine-3-carbonyl]sulfamoyl]-2-pyridyl]amino]-4-methyl-pentyl]-2,2-dimethyl-pyrrolidine-1-carboxylate

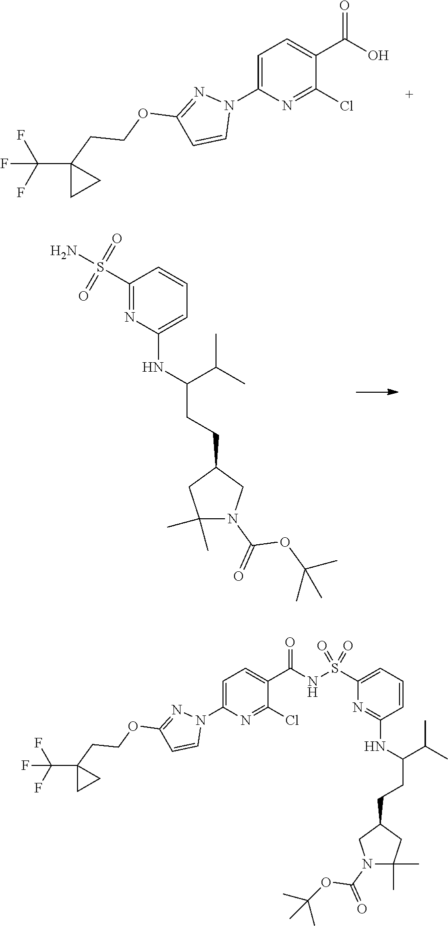

To a solution of 2-chloro-6-[3-[2-[1-(trifluoromethyl)cyclopropyl]ethoxy]pyrazol-1-yl]pyridine-3-carboxylic acid (175 mg, 0.47 mmol) in THF (5 mL) was added CDI (77 mg, 0.47 mmol) (recrystallized from THF) and the mixture was stirred at rt for 3 h. Then tert-butyl (4S)-2,2-dimethyl-4-[4-methyl-3-[(6-sulfamoyl-2-pyridyl)amino]pentyl]pyrrolidine-1-carboxylate (151.5 mg, 0.33 mmol) was added followed by DBU (200 μL, 1.34 mmol) and the resulting mixture was stirred for 16 h at rt. The reaction was diluted with ethyl acetate and washed with a saturated aqueous sodium bicarbonate solution. The organic layer was further washed with 10% citric acid solution followed by brine. The organics were separated, dried over sodium sulfate, evaporated and then purified on silica gel chromatography (40 gram column) using a gradient from 100% hexanes to 100% ethyl acetate followed by a second silica gel column (24 gram column) using a gradient from 100% dichloromethane to 15% methanol in dichloromethane to afford tert-butyl (4S)-4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]-4-methyl-pentyl]-2,2-dimethyl-pyrrolidine-1-carboxylate (155 mg, 57%) as an off-white solid. ¹H NMR (500 MHz, DMSO-d₆) δ 12.75 (s, 1H), 8.41 (t, J=3.9 Hz, 1H), 8.09 (d, J=7.8 Hz, 1H), 7.71 (d, J=8.4 Hz, 1H), 7.59 (t, J=7.8 Hz, 1H), 7.13 (d, J=7.2 Hz, 1H), 6.98 (s, 1H), 6.77 (d, J=8.5 Hz, 1H), 6.19 (d, J=3.1 Hz, 1H), 4.35 (t, J 7.2 Hz, 2H), 3.87 (s, 1H), 3.58-3.41 (m, 1H), 2.69 (q, J=10.1, 9.6 Hz, 1H), 2.09 (d, J 3.9 Hz, 4H), 1.98 (s, 1H), 1.77 (dd, J=12.9, 6.6 Hz, 2H), 1.49 (s, 1H), 1.36 (d, J=15.9 Hz, 9H), 1.31-1.26 (m, 4H), 1.25-1.19 (m, 1H), 1.16 (s, 3H), 0.96 (d, J=5.1 Hz, 2H), 0.89 (s, 2H), 0.83 (d, J=6.8 Hz, 6H). ESI-MS m/z calc. 811.31055, found 812.6 (M+1)⁺; Retention time: 2.49 minutes (LC method A).

### Step 5: (14S)-12,12-Dimethyl-17-(propan-2-yl)-8-(3-{2-[1-(trifluoromethyl)cyclo propyl]ethoxy}-1H-pyrazol-1-yl)-2λ⁶-thia-3,9,11,18,23-pentaazatetracyclo [17.3.1.111,14.05,10]tetracosa-1(23),5(10),6,8,19,21-hexaene-2,2,4-trione (diastereomer 1, Compound 358) and (14S)-12,12-dimethyl-17-(propan-2-yl)-8-(3-{2-[1-(trifluoromethyl)cyclo propyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (diastereomer 2, Compound 357)

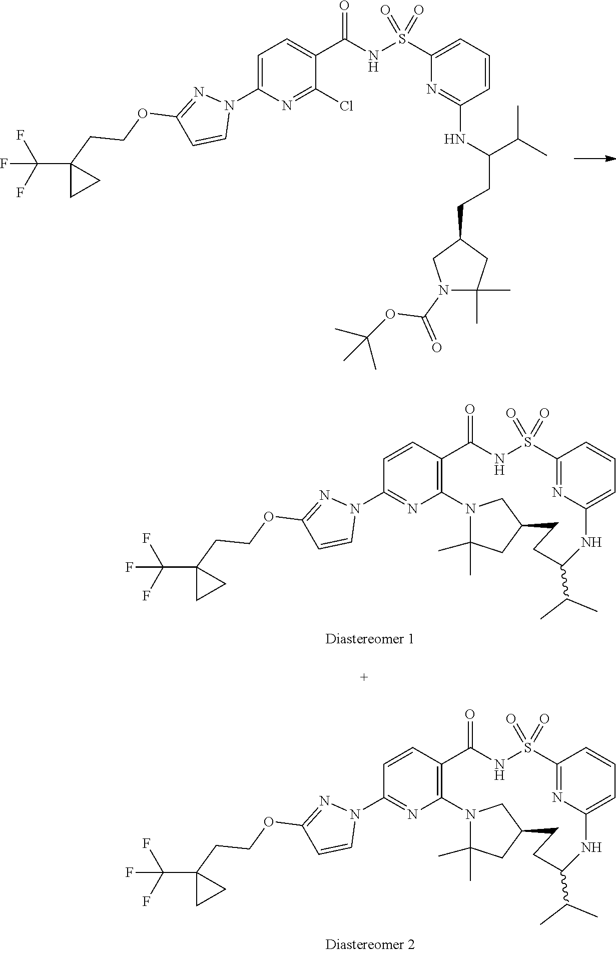 [SMILES: CC(C)C(CC[C@@H]1CN(C(=O)OC(C)(C)C)C(C)(C)C1)Nc1cccc(S(=O)(=O)NC(=O)c2ccc(-n3ccc(OCCC4(C(F)(F)F)CC4)n3)nc2Cl)n1 ; CC(C)C1CC[C@@H]2CN(c3nc(-n4ccc(OCCC5(C(F)(F)F)CC5)n4)ccc3C(=O)NS(=O)(=O)c3cccc(n3)N1)C(C)(C)C2 ; CC(C)C1CC[C@@H]2CN(c3nc(-n4ccc(OCCC5(C(F)(F)F)CC5)n4)ccc3C(=O)NS(=O)(=O)c3cccc(n3)N1)C(C)(C)C2]

tert-Butyl (4S)-4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]-4-methyl-pentyl]-2,2-dimethyl-pyrrolidine-1-carboxylate (145 mg, 0.18 mmol) was dissolved in DCM (4.0 mL) and to the mixture was added HCl (2 mL of 4 M in dioxane, 8.0 mmol) and stirred at room temperature. After 1 h, the mixture was evaporated to dryness, then diluted with diethyl ether (5 mLs×2), and reconcentrated. The material was then placed on the high vacuum pump for 2 h to afford the intermediate 2-chloro-N-[[6-[[1-[2-[(3S)-5,5-dimethylpyrrolidin-3-yl]ethyl]-2-methyl-propyl]amino]-2-pyridyl]sulfonyl]-6-[3-[2-[1-(trifluoromethyl)cyclopropyl]ethoxy]pyrazol-1-yl]pyridine-3-carboxamide (hydrochloride salt) as an off-white solid. ESI-MS m/z calc. 711.2581, found 712.2 (M+1)⁺; Retention time: 1.76 minutes.

The material was combined with K₂CO₃ (175 mg, 1.266 mmol), 3 Å molecular sieves and DMSO (5 mL) in a vial, which was purged with nitrogen, capped, heated to 155° C. and stirred for 18h. The mixture was cooled to room temperature. The mixture was filtered and concentrated under a stream of nitrogen to give a residue which was purified by reverse-phase preparative chromatography utilizing a C₁₈ column and HPLC-MS method 30-99% A1B1 (Acetonitrile-Water+5 mmolar HCl, 15 minute method×4 injections) to afford two products:

Diastereomer 1, more polar, tan solid: (14S)-12,12-dimethyl-17-(propan-2-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (25.55 mg, 42%) ESI-MS m/z calc. 675.28143, found 676.2 (M+1)⁺; Retention time: 2.33 minutes (LC method A).

Diastereomer 2, less polar, tan solid: (14S)-12,12-dimethyl-17-(propan-2-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (18.18 mg, 30%). ¹H NMR (500 MHz, DMSO-d₆) δ 12.47 (s, 1H), 8.21 (d, J=2.8 Hz, 1H), 7.67 (d, J=8.3 Hz, 1H), 7.58 (t, J=7.9 Hz, 1H), 7.16 (d, J=7.3 Hz, 1H), 7.09 (s, 1H), 6.90 (d, J=8.2 Hz, 1H), 6.78 (d, J=8.6 Hz, 1H), 6.11 (d, J=2.8 Hz, 1H), 4.32 (t, J=7.0 Hz, 2H), 3.35 (s, 2H), 3.13 (s, 1H), 2.92 (s, 1H), 2.27 (s, 1H), 2.08 (t, J=7.1 Hz, 2H), 2.01 (s, 1H), 1.91-1.83 (m, 1H), 1.67 (s, 2H), 1.56 (d, J=28.2 Hz, 7H), 1.20 (s, 1H), 0.96 (d, J=4.9 Hz, 2H), 0.89 (t, J=6.5 Hz, 8H). ESI-MS m/z calc. 675.28143, found 676.2 (M+1)⁺; Retention time: 2.4 minutes (LC method A).

### Example 122: Preparation of (14S)-8-[3-(2-{Dispiro[2.0.2.1]heptan-7-yl}ethoxy)-1H-pyrazol-1-yl]-12,12-dimethyl-17-(propan-2-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 1, Compound 354), and (14S)-8-[3-(2-{dispiro[2.0.2.1]heptan-7-yl}ethoxy)-1H-pyrazol-1-yl]-12,12-dimethyl-17-(propan-2-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 2, Compound 353)

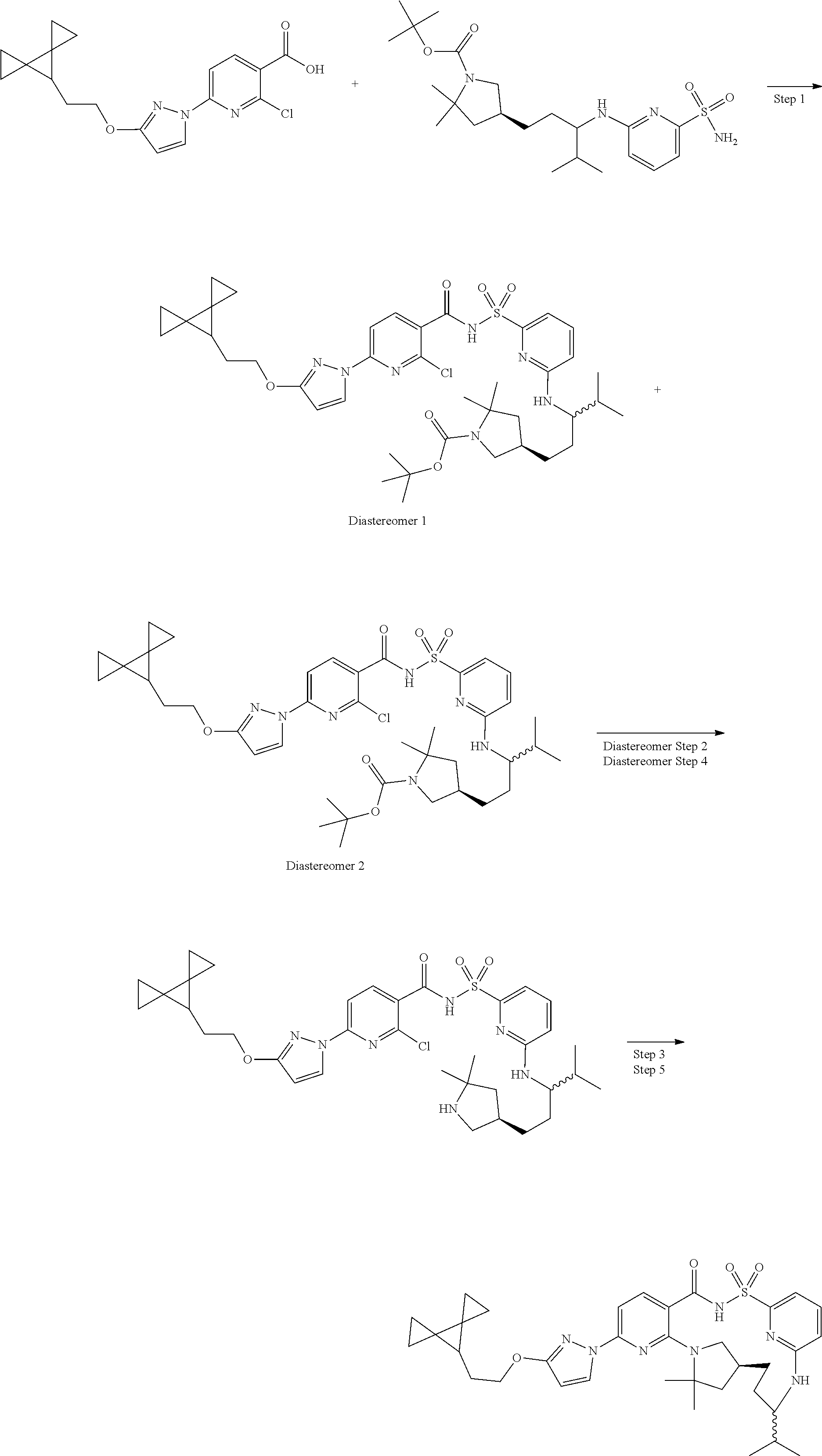

### Step 1: tert-Butyl (4R)-4-[3-[[6-[[2-chloro-6-[3-(2-dispiro[2.0.2.1]heptan-7-ylethoxy)pyrazol-1-yl]pyridine-3-carbonyl]sulfamoyl]-2-pyridyl]amino]-4-methyl-pentyl]-2,2-dimethyl-pyrrolidine-1-carboxylate, diastereomer 1, and tert-butyl (4R)-4-[3-[[6-[[2-chloro-6-[3-(2-dispiro[2.0.2.1]heptan-7-ylethoxy)pyrazol-1-yl]pyridine-3-carbonyl]sulfamoyl]-2-pyridyl]amino]-4-methyl-pentyl]-2,2-dimethyl-pyrrolidine-1-carboxylate, diastereomer 2

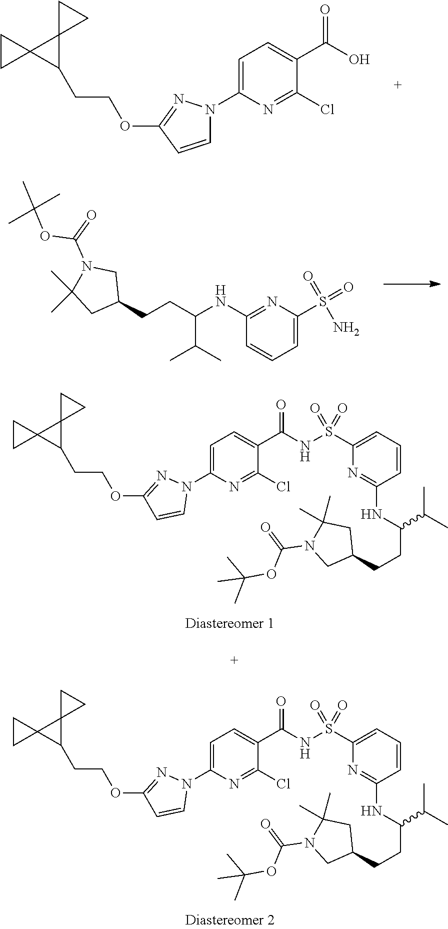 [SMILES: O=C(O)c1ccc(-n2ccc(OCCC3C4(CC4)C34CC4)n2)nc1Cl ; CC(C)C(CC[C@@H]1CN(C(=O)OC(C)(C)C)C(C)(C)C1)Nc1cccc(S(N)(=O)=O)n1 ; CC(C)C(CC[C@H]1CN(C(=O)OC(C)(C)C)C(C)(C)C1)Nc1cccc(S(=O)(=O)NC(=O)c2ccc(-n3ccc(OCCC4C5(CC5)C45CC5)n3)nc2Cl)n1 ; CC(C)C(CC[C@H]1CN(C(=O)OC(C)(C)C)C(C)(C)C1)Nc1cccc(S(=O)(=O)NC(=O)c2ccc(-n3ccc(OCCC4C5(CC5)C45CC5)n3)nc2Cl)n1]

To a solution of 2-Chloro-6-[3-(2-dispiro[2.0.2.1]heptan-7-ylethoxy)pyrazol-1-yl]pyridine-3-carboxylic acid (100 mg, 0.28 mmol) in THF (600 μL) was added CDI (56.28 mg, 0.35 mmol) (recrystallized from THF) and the mixture was stirred at rt for 2 h then tert-butyl (4S)-2,2-dimethyl-4-[4-methyl-3-[(6-sulfamoyl-2-pyridyl)amino]pentyl]pyrrolidine-1-carboxylate (132.7 mg, 0.29 mmol) was added as a solution in THF (600 μL) followed by DBU (132.8 mg, 0.8723 mmol) and the resulting mixture was stirred for 3 h at room temperature. The reaction was diluted with water and EtOAc then HCl (310 μL, of 6 M, 1.864 mmol) was added, aqueous layer was then pH=1. The layers were separated and the organic layer was washed with water and brine (1×) then dried over sodium sulfate and concentrated to a white foam which was filtered and purified using a reverse phase HPLC-MS method using a LuNa C₁₈ (2) column (75×30 mm, 5 μm particle size) sold by Phenomenex (pn: 00C-4252-U0-AX), and a dual gradient run from 50-99% mobile phase B over 15.0 minutes (mobile phase A=H₂O (5 mM HCl), mobile phase B=acetonitrile, flow rate=50 mL/min, injection volume=950 μL and column temperature=25° C.), giving as diastereomer 1 to elute: tert-butyl (4R)-4-[3-[[6-[[2-chloro-6-[3-(2-dispiro[2.0.2.1]heptan-7-ylethoxy)pyrazol-1-yl]pyridine-3-carbonyl]sulfamoyl]-2-pyridyl]amino]-4-methyl-pentyl]-2,2-dimethyl-pyrrolidine-1-carboxylate (50 mg, 45%) and as the diastereomer 2 to elute, tert-butyl (4R)-4-[3-[[6-[[2-chloro-6-[3-(2-dispiro[2.0.2.1]heptan-7-ylethoxy)pyrazol-1-yl]pyridine-3-carbonyl]sulfamoyl]-2-pyridyl]amino]-4-methyl-pentyl]-2,2-dimethyl-pyrrolidine-1-carboxylate (55 mg, 50%) both as white solids.

### Step 2: 2-Chloro-N-[[6-[[1-[2-[(3R)-5,5-dimethylpyrrolidin-3-yl]ethyl]-2-methyl-propyl]amino]-2-pyridyl]sulfonyl]-6-[3-(2-dispiro[2.0.2.1]heptan-7-ylethoxy)pyrazol-1-yl]pyridine-3-carboxamide, diastereomer 1

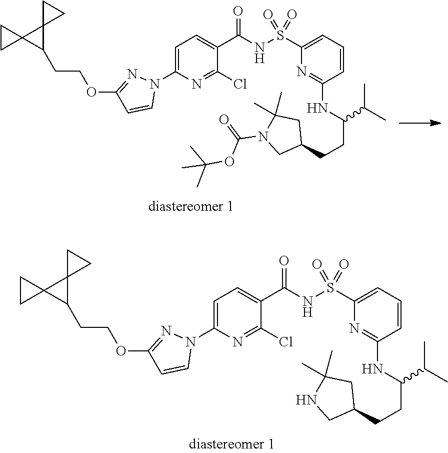 [SMILES: CC(C)C(CC[C@H]1CN(C(=O)OC(C)(C)C)C(C)(C)C1)Nc1cccc(S(=O)(=O)NC(=O)c2ccc(-n3ccc(OCCC4C5(CC5)C45CC5)n3)nc2Cl)n1 ; CC(C)C(CC[C@H]1CNC(C)(C)C1)Nc1cccc(S(=O)(=O)NC(=O)c2ccc(-n3ccc(OCCC4C5(CC5)C45CC5)n3)nc2Cl)n1]

tert-Butyl (4R)-4-[3-[[6-[[2-chloro-6-[3-(2-dispiro[2.0.2.1]heptan-7-ylethoxy)pyrazol-1-yl]pyridine-3-carbonyl]sulfamoyl]-2-pyridyl]amino]-4-methyl-pentyl]-2,2-dimethyl-pyrrolidine-1-carboxylate (50 mg, 0.06278 mmol) (diastereomer 1) was dissolved in DCM (218.2 μL) and to the mixture was added TFA (193.6 μL, 2.51 mmol) and the mixture was stirred at room temperature for 3 h. Concentrated mixture to dryness under reduced pressure, added 1 mL of toluene and removed by rotary evaporation (45° C. water bath). Again added 1 mL of toluene and removed by rotary evaporation (45° C. water bath) then dried on the high vacuum giving 2-chloro-N-[[6-[[1-[2-[(3R)-5,5-dimethylpyrrolidin-3-yl]ethyl]-2-methyl-propyl]amino]-2-pyridyl]sulfonyl]-6-[3-(2-dispiro[2.0.2.1]heptan-7-ylethoxy)pyrazol-1-yl]pyridine-3-carboxamide (trifluoroacetate salt) as a white solid (50.87 mg, 100%). ESI-MS m/z calc. 695.30206, found 696.7 (M+1)⁺; Retention time: 0.75 minutes (LC method D).

### Step 3: (14S)-8-13-(2-{Dispiro[2.0.2.11heptan-7-yl}ethoxy)-1H-pyrazol-1-yl]-12,12-dimethyl-17-(propan-2-yl)-2λ⁶-thia-3,9,11,18,23-pentaazatetracyclo[17.3.1.111,14.05,10]tetracosa-1(22),5,7,9,19(23),20-hexaene-2,2,4-trione, diastereomer 1 (Compound 56)

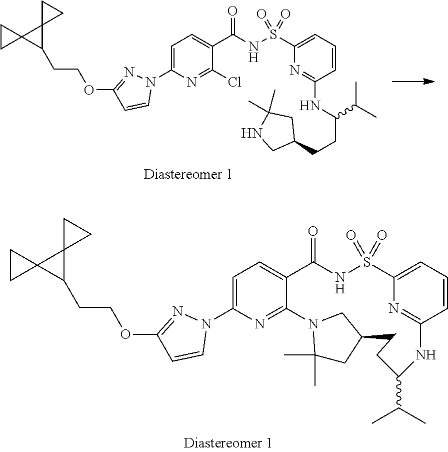 [SMILES: CC(C)C(CC[C@H]1CNC(C)(C)C1)Nc1cccc(S(=O)(=O)NC(=O)c2ccc(-n3ccc(OCCC4C5(CC5)C45CC5)n3)nc2Cl)n1 ; CC(C)C1CC[C@@H]2CN(c3nc(-n4ccc(OCCC5C6(CC6)C56CC6)n4)ccc3C(=O)NS(=O)(=O)c3cccc(n3)N1)C(C)(C)C2]

To a solution of 2-chloro-N-[[6-[[1-[2-[(3R)-5,5-dimethylpyrrolidin-3-yl]ethyl]-2-methyl-propyl]amino]-2-pyridyl]sulfonyl]-6-[3-(2-dispiro[2.0.2.1]heptan-7-ylethoxy)pyrazol-1-yl]pyridine-3-carboxamide, diastereomer 1 (trifluoroacetate salt) (50.87 mg, 0.06278 mmol) in NMP (3 mL) was added potassium carbonate (60.76 mg, 0.4396 mmol). The mixture was purged with nitrogen for 5 min. The mixture was heated at 165° C. for 16h. The mixture was cooled to room temperature, diluted with 1N HCl and extracted with EtOAc (1×). The organic phase was washed with brine (1×), dried (sodium sulfate), filtered and concentrated to a brown oil which was filtered and purified using a reverse phase HPLC-MS method using a LuNa C₁₈ (2) column (75×30 mm, 5 μm particle size) sold by Phenomenex (pn: 00C-4252-U0-AX), and a dual gradient run from 50-99% mobile phase B over 15.0 minutes (mobile phase A=H₂O (5 mM HCl), mobile phase B=acetonitrile, flow rate=50 mL/min, injection volume=950 μL and column temperature=25° C.) giving (14S)-8-[3-(2-{dispiro[2.0.2.1]heptan-7-yl}ethoxy)-1H-pyrazol-1-yl]-12,12-dimethyl-17-(propan-2-yl)-2λ⁶-thia-3,9,11,18,23-pentaazatetracyclo[17.3.1.111,14.05,10]tetracosa-1(22),5,7,9,19(23),20-hexaene-2,2,4-trione diastereomer 1 as a white solid. (28 mg, 68%). ¹H NMR (500 MHz, DMSO-d₆) δ 12.43 (s, 1H), 8.19 (d, J=2.8 Hz, 1H), 7.66 (d, J=8.2 Hz, 1H), 7.58 (t, J=7.9 Hz, 1H), 7.16 (d, J=7.2 Hz, 1H), 7.07 (s, 1H), 6.89 (d, J=8.1 Hz, 1H), 6.78 (d, J=8.5 Hz, 1H), 6.07 (d, J=2.7 Hz, 1H), 4.21 (t, J=6.7 Hz, 2H), 3.13 (s, 1H), 2.92 (s, 1H), 2.27 (d, J 8.8 Hz, 1H), 2.00 (s, 1H), 1.88 (dd, J=12.0, 5.9 Hz, 1H), 1.81 (q, J=6.7 Hz, 2H), 1.67 (s, 2H), 1.59 (s, 3H), 1.57 (s, 1H), 1.54 (s, 3H), 1.47 (t, J=6.6 Hz, 1H), 1.20 (s, 1H), 0.89 (t, J=6.7 Hz, 6H), 0.87-0.78 (m, 4H), 0.64 (dd, J=8.1, 4.1 Hz, 2H), 0.50 (dd, J=8.4, 4.2 Hz, 2H). Two protons in the NMR are obscured by water or DMSO-d₆ signals. ESI-MS m/z calc. 659.3254, found 660.7 (M+1)⁺; Retention time: 1.75 minutes (LC method M).

### Step 4: 2-Chloro-N-[[6-[[1-[2-[(3R)-5,5-dimethylpyrrolidin-3-yl]ethyl]-2-methyl-propyl]amino]-2-pyridyl]sulfonyl]-6-[3-(2-dispiro[2.0.2.1]heptan-7-ylethoxy)pyrazol-1-yl]pyridine-3-carboxamide, diastereomer 2

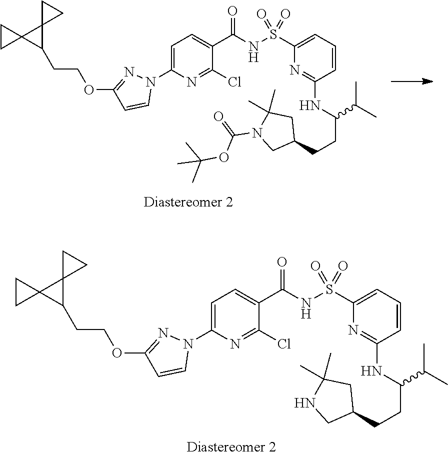 [SMILES: CC(C)C(CC[C@H]1CN(C(=O)OC(C)(C)C)C(C)(C)C1)Nc1cccc(S(=O)(=O)NC(=O)c2ccc(-n3ccc(OCCC4C5(CC5)C45CC5)n3)nc2Cl)n1 ; CC(C)C(CC[C@H]1CNC(C)(C)C1)Nc1cccc(S(=O)(=O)NC(=O)c2ccc(-n3ccc(OCCC4C5(CC5)C45CC5)n3)nc2Cl)n1]

tert-Butyl (4R)-4-[3-[6-[[2-chloro-6-[3-(2-dispiro[2.0.2.1]heptan-7-ylethoxy)pyrazol-1-yl]pyridine-3-carbonyl]sulfamoyl]-2-pyridyl]aminol-4-methyl-pentyl]-2,2-dimethyl-pyrrolidine-1-carboxylate (55 mg, 0.069 mmol) (diastereomer 2) was dissolved in DCM (240 μL) and to the mixture was added TFA (213 μL, 2.76 mmol) and the mixture was stirred at room temperature for 3 h. Concentrated mixture to dryness under reduced pressure, added 1 mL of toluene and removed by rotary evaporation (45° C. water bath). Again added 1 mL of toluene and removed by rotary evaporation (45° C. water bath) then dried on the high vacuum giving 2-chloro-N-[[6-[[1-[2-[(3R)-5,5-dimethylpyrrolidin-3-yl]ethyl]-2-methyl-propyl]amino]-2-pyridyl]sulfonyl]-6-[3-(2-dispiro[2.0.2.1]heptan-7-ylethoxy)pyrazol-1-yl]pyridine-3-carboxamide, diastereomer 2 (trifluoroacetate salt) as a white solid (55.96 mg, 100%). ESI-MS m/z calc. 695.30206, found 696.7 (M+1)⁺; Retention time: 0.75 minutes (LC method D).

### Step 5: (14S)-8-13-(2-{Dispiro[2.0.2.11heptan-7-yl}ethoxy)-1H-pyrazol-1-yl]-12,12-dimethyl-17-(propan-2-yl)-2λ⁶-thia-3,9,11,18,23-pentaazatetracyclo[17.3.1.111,14.05,10]tetracosa-1(22),5,7,9,19(23),20-hexaene-2,2,4-trione (Compound 55)

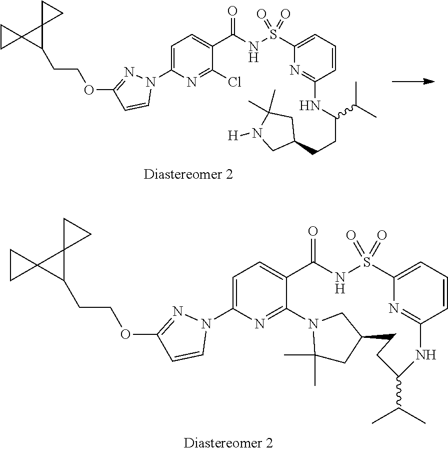 [SMILES: CC(C)C(CC[C@H]1CNC(C)(C)C1)Nc1cccc(S(=O)(=O)NC(=O)c2ccc(-n3ccc(OCCC4C5(CC5)C45CC5)n3)nc2Cl)n1 ; CC(C)C1CC[C@@H]2CN(c3nc(-n4ccc(OCCC5C6(CC6)C56CC6)n4)ccc3C(=O)NS(=O)(=O)c3cccc(n3)N1)C(C)(C)C2]

To a solution of 2-chloro-N-[[6-[[1-[2-[(3R)-5,5-dimethylpyrrolidin-3-yl]ethyl]-2-methyl-propyl]amino]-2-pyridyl]sulfonyl]-6-[3-(2-dispiro[2.0.2.1]heptan-7-ylethoxy)pyrazol-1-yl]pyridine-3-carboxamide, diastereomer 2 (trifluoroacetate salt) (55.96 mg, 0.069 mmol) in NMP (3 mL) was added potassium carbonate (66.84 mg, 0.4836 mmol). The mixture was purged with nitrogen for 5 min. The mixture was heated at 165° C. for 16h. The mixture was cooled to room temperature, diluted with 1N HCl and extracted with EtOAc (1×). The organic phase was washed with brine (1×), dried (sodium sulfate), filtered and concentrated to a brown oil which was filtered and purified using a reverse phase HPLC-MS method using a LuNa C₁₈ (2) column (75×30 mm, 5 μm particle size) sold by Phenomenex (pn: 00C-4252-U0-AX), and a dual gradient run from 50-99% mobile phase B over 15.0 minutes (mobile phase A=H₂O (5 mM HCl), mobile phase B=acetonitrile, flow rate=50 mL/min, injection volume=950 μL and column temperature=25° C.) giving (14S)-8-[3-(2-{dispiro[2.0.2.1]heptan-7-yl}ethoxy)-1H-pyrazol-1-yl]-12,12-dimethyl-17-(propan-2-yl)-2λ⁶-thia-3,9,11,18,23-pentaazatetracyclo[17.3.1.111,14.05,10]tetracosa-1(22),5,7,9,19(23),20-hexaene-2,2,4-trione, diastereomer 2 as a white solid (27.2 mg, 60%). ¹H NMR (500 MHz, DMSO-d₆) δ 12.47 (s, 1H), 8.21 (d, J=2.8 Hz, 1H), 7.83 (d, J=8.2 Hz, 1H), 7.56 (t, J=7.8 Hz, 1H), 7.02 (d, J=7.1 Hz, 1H), 6.92 (dd, J=8.7, 4.8 Hz, 2H), 6.74 (d, J=8.5 Hz, 1H), 6.09 (d, J=2.7 Hz, 1H), 4.22 (t, J=6.7 Hz, 2H), 3.96 (s, 1H), 3.15 (dd, J=10.6, 6.9 Hz, 1H), 2.68 (t, J=10.6 Hz, 1H), 2.08 (s, 1H), 1.83 (dq, J=19.6, 6.6, 6.0 Hz, 3H), 1.64 (d, J=6.1 Hz, 2H), 1.61 (s, 3H), 1.57 (d, J=12.5 Hz, 2H), 1.51 (s, 3H), 1.48 (t, J=6.7 Hz, 2H), 1.32-1.21 (m, 1H), 0.92-0.79 (m, 10H), 0.68-0.61 (m, 2H), 0.50 (d, J=8.5 Hz, 2H). ESI-MS m/z calc. 659.3254, found 660.7 (M+1)⁺; Retention time: 1.62 minutes (LC method M).

### Example 123: Preparation of 12,12-Dimethyl-17-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 388 (enantiomer 1), Compound 387 (enantiomer 2), Compound 384 (enantiomer 3), and Compound 383 (enantiomer 4)

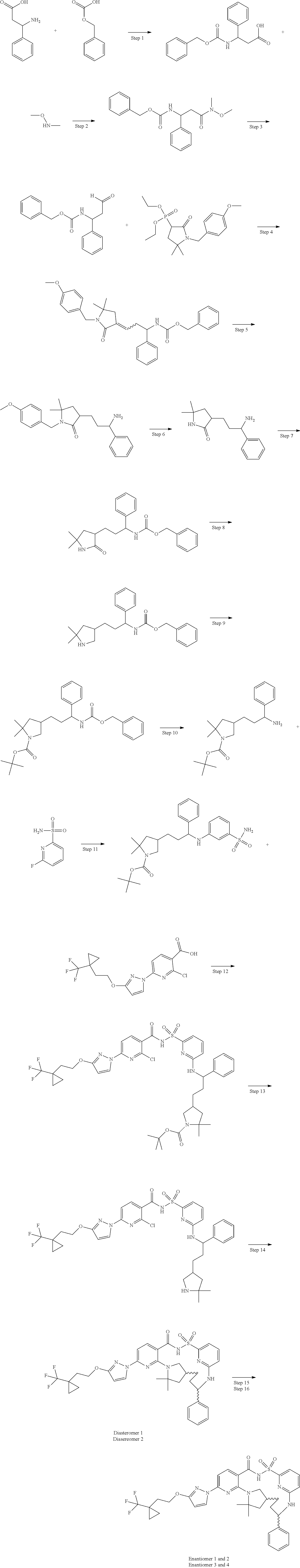

### Step 1: 3-(Benzyloxycarbonylamino)-3-phenyl-propanoic acid

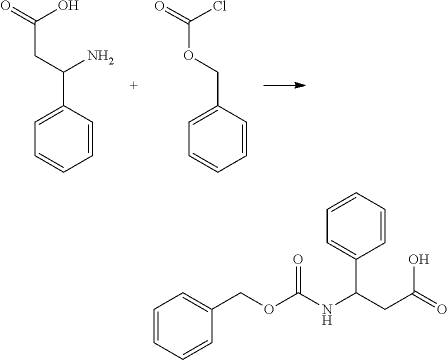 [SMILES: NC(CC(=O)O)c1ccccc1 ; O=C(Cl)OCc1ccccc1 ; O=C(O)CC(NC(=O)OCc1ccccc1)c1ccccc1]

3-Amino-3-phenyl-propanoic acid (30 g, 177.98 mmol) was mixed with sodium hydroxide (180 mL of 1 M, 180.00 mmol) and cooled in ice water bath. Cbz chloride (30 mL, 188.87 mmol) was added along with sodium hydroxide (180 mL of 1 M, 180.00 mmol). The reaction mixture was stirred at rt for 16 hours. The mixture was extracted with diethyl ether (450 mL). Layers were separated and the aqueous layer was cooled in an ice water bath. Conc. HCl was added until the pH of the solution reached 1. The solution was then extracted with DCM (500 mL×3). The combined organics was washed by brine (800 mL), dried over sodium sulfate, filtered and concentrated to afford 3-(benzyloxycarbonylamino)-3-phenyl-propanoic acid (40.8 g, 73%) as a white solid. ESI-MS m/z calc. 299.1158, found 300.5 (M+1)⁺; Retention time: 3.26 minutes (LC method B).

### Step 2: Benzyl N-[3-[methoxy(methyl)amino]-3-oxo-1-phenyl-propyl]carbamate

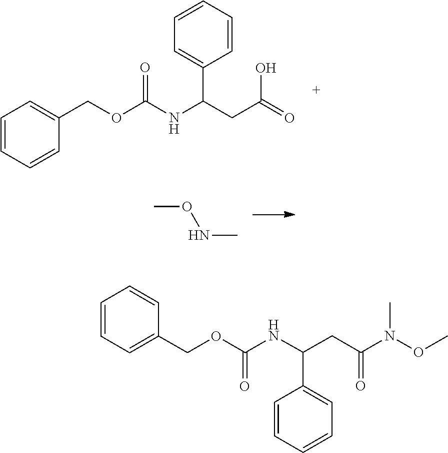

To a 500 mL round bottom flask equipped with stir bar was added 3-(benzyloxycarbonylamino)-3-phenyl-propanoic acid (9.5 g, 28.57 mmol), dichloromethane (50 mL) and tetrahydrofuran (200 mL). To this stirred solution was added 1,1′-carbonyl diimidazone (6 g, 36.26 mmol) and was stirred at room temperature for 40 minutes. At this time, N,O-dimethylhydroxylamine (hydrochloride salt) (3.6 g, 36.17 mmol) was added. Then the mixture was stirred at room temperature for 19 hours. The reaction mixture was combined with another reaction run on 3.5 g of 3-(benzyloxycarbonylamino)-3-phenyl-propanoic acid and it was quenched with 1M HCl (100 mL), and the solvents were removed. The aqueous layer was extracted with ethyl acetate (3×100 mL) and a 1:1 mixture of brine and saturated sodium bicarbonate solution (200 mL). The organic layer was dried over sodium sulfate and concentrated to afford benzyl N-[3-[methoxy(methyl)amino]-3-oxo-1-phenyl-propyl]carbamate (13.5 g, adjusted yield: 96%) as a white solid. ESI-MS m/z calc. 342.16, found 343.4 (M+1)⁺; Retention time: 3.22 minutes (LC method B).

### Step 3: Benzyl N-(3-oxo-1-phenyl-propyl)carbamate

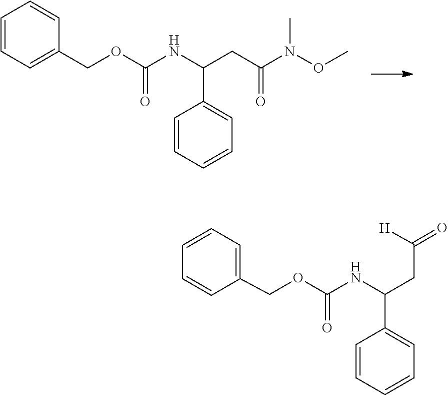 [SMILES: CON(C)C(=O)CC(NC(=O)OCc1ccccc1)c1ccccc1 ; O=CCC(NC(=O)OCc1ccccc1)c1ccccc1]

To a solution of benzyl N-[3-[methoxy(methyl)amino]-3-oxo-1-phenyl-propyl]carbamate (61.95 g, 171.89 mmol) in tetrahydrofuran (800 mL) was added LiAlH₄ (2M in THF, 130 mL, 260 mmol) at 0° C. The reaction mixture was stirred for 50 minutes gradually from 0° C. to 25° C. The mixture was quenched with a 5% solution of HCl (800 mL) and extracted with ethyl acetate (3×600 mL). The organic layer was dried over sodium sulfate and concentrated to give benzyl N-(3-oxo-1-phenyl-propyl)carbamate (48.38 g, 94%). ESI-MS m/z calc. 283.1208, found 284.4 (M+1)⁺; Retention time: 3.25 minutes (LC method B).

### Step 4: Benzyl N-[3-[1-[(4-methoxyphenyl)methyl]-5,5-dimethyl-2-oxo-pyrrolidin-3-ylidene]-1-phenyl-propyl]carbamate

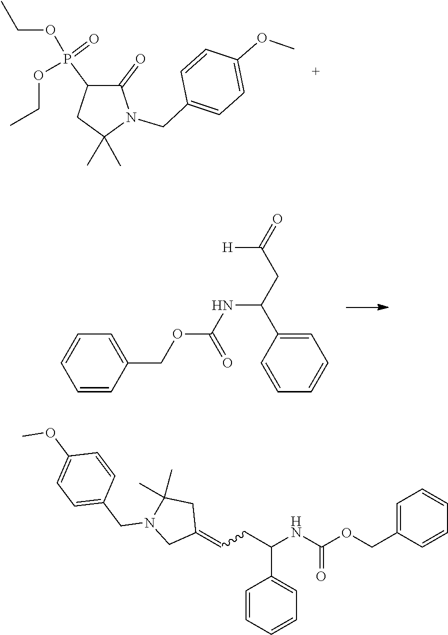

3-Diethoxyphosphoryl-1-[(4-methoxyphenyl)methyl]-5,5-dimethyl-pyrrolidin-2-one (5 g, 12.86 mmol) was dissolved in tetrahydrofuran (95 mL) and cooled in an ice water bath. 60% NaH in mineral oil (1.2 g, 30 mmol) was added and the resulting mixture was stirred for thirty minutes. Benzyl N-(3-oxo-1-phenyl-propyl)carbamate (6.17 g, 17.422 mmol) was dissolved in tetrahydrofuran (35 mL), the resulting solution was added in a slow stream to the cooled phosphonate solution. The resulting mixture was stirred at 0° C. and allowed to warm to room temperature and stir for sixteen hours. The reaction mixture was quenched by adding water (150 mL) and extracted with ethyl acetate (3×200 mL). The organic layer was washed with brine, dried over sodium sulfate and concentrated. The residue (combined with another crude product from the same reaction using 2 g of 3-Diethoxyphosphoryl-1-[(4-methoxyphenyl)methyl]-5,5-dimethyl-pyrrolidin-2-one) was purified by silica gel column chromatography using 30-70% hexanes-ethyl acetate to afford benzyl N-[3-[1-[(4-methoxyphenyl)methyl]-5,5-dimethyl-2-oxo-pyrrolidin-3-ylidene]-1-phenyl-propyl]carbamate as an off white solid (3.68 g, adjusted yield 40%). ESI-MS m/z calc. 498.2519, found 499.7 (M+1)⁺; Retention time: 3.85 minutes (LC method B).

### Step 5: 3-(3-Amino-3-phenyl-propyl)-1-[(4-methoxyphenyl)methyl]-5,5-dimethyl-pyrrolidin-2-one

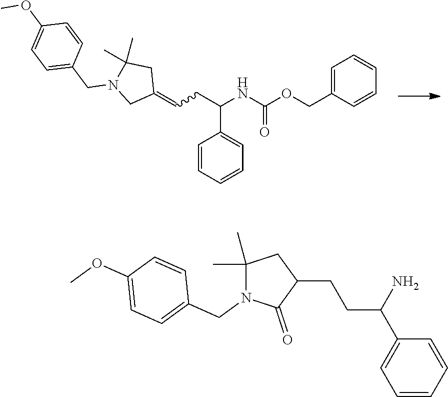 [SMILES: COc1ccc(CN2CC(=CCC(NC(=O)OCc3ccccc3)c3ccccc3)CC2(C)C)cc1 ; COc1ccc(CN2C(=O)C(CCC(N)c3ccccc3)CC2(C)C)cc1]

To a solution of benzyl N-[3-[1-[(4-methoxyphenyl)methyl]-5,5-dimethyl-2-oxo-pyrrolidin-3-ylidene]-1-phenyl-propyl]carbamate (11.32 g, 21.57 mmol) in ethyl acetate (90 mL), tetrahydrofuran (10 mL) and acetic acid (6 mL) was added 10% palladium on carbon (5.0 g). The mixture was reacted in a Parr shaker at 60 psi of hydrogen for 2 hours. The reaction mixture was filtered through a Celite pad. The filtrate was concentrated to give crude 3-(3-amino-3-phenyl-propyl)-1-[(4-methoxyphenyl)methyl]-5,5-dimethyl-pyrrolidin-2-one (Acetate salt) (12.5 g, 129%) ESI-MS m/z calc. 366.2307, found 367.5 (M+1)⁺; Retention time: 3.02 minutes (LC method B).

### Step 6: 3-(3-Amino-3-phenyl-propyl)-5,5-dimethyl-pyrrolidin-2-one

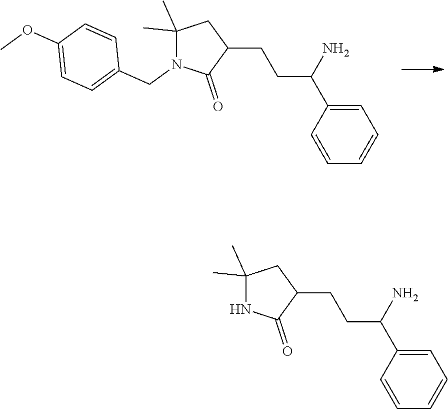 [SMILES: COc1ccc(CN2C(=O)C(CCC(N)c3ccccc3)CC2(C)C)cc1 ; CC1(C)CC(CCC(N)c2ccccc2)C(=O)N1]

A solution of 3-(3-amino-3-phenyl-propyl)-1-[(4-methoxyphenyl)methyl]-5,5-dimethyl-pyrrolidin-2-one (Acetate salt) (6.75 g, 15.03 mmol) in TFA (18 mL) was heated at 125° C. in a microwave reactor for 11 hours. The result solution was concentrated to remove TFA. The residue was diluted with dichloromethane (150 mL) and basified by saturated sodium bicarbonate (150 mL). The organic layer was washed with brine, dried over sodium sulfate and concentrated to give 3-(3-amino-3-phenyl-propyl)-5,5-dimethyl-pyrrolidin-2-one (2.29 g, 59%) as an orange gel. ESI-MS m/z calc. 246.1732, found 247.6 (M+1)⁺; Retention time: 2.44 minutes (LC method B).

### Step 7: Benzyl N-[3-(5,5-dimethyl-2-oxo-pyrrolidin-3-yl)-1-phenyl-propyl]carbamate

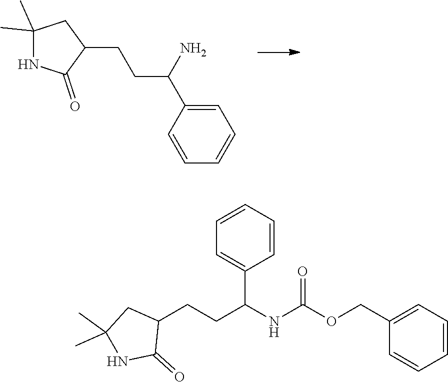 [SMILES: CC1(C)CC(CCC(N)c2ccccc2)C(=O)N1 ; CC1(C)CC(CCC(NC(=O)OCc2ccccc2)c2ccccc2)C(=O)N1]

To a solution of 3-(3-amino-3-phenyl-propyl)-5,5-dimethyl-pyrrolidin-2-one (2.29 g, 8.83 mmol) in triethylamine 11 mL, 78.921 mmol) and dichloromethane (80 mL) was added Cbz chloride (1.5 mL, 9.44 mmol) at 0° C. The mixture was stirred at room temperature for 24 hours, and then 1N HCl (200 mL) and dichloromethane (200 mL) were added. The organic layer was washed by brine, dried over sodium sulfate and concentrated to give crude benzyl N-[3-(5,5-dimethyl-2-oxo-pyrrolidin-3-yl)-1-phenyl-propyl]carbamate (3.56 g, 101%) as an orange gel. ESI-MS m/z calc. 380.21, found 381.6 (M+1)⁺; Retention time: 3.52 minutes (LC method B).

### Step 8: Benzyl N-[3-(5,5-dimethylpyrrolidin-3-yl)-1-phenyl-propyl]carbamate

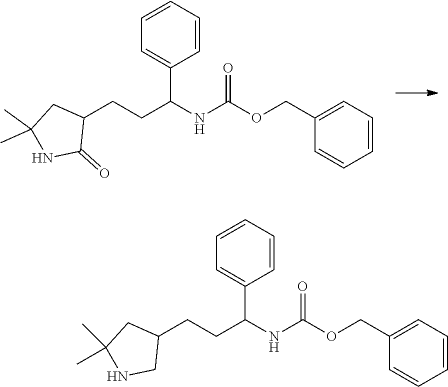 [SMILES: CC1(C)CC(CCC(NC(=O)OCc2ccccc2)c2ccccc2)C(=O)N1 ; CC1(C)CC(CCC(NC(=O)OCc2ccccc2)c2ccccc2)CN1]

To a solution of benzyl N-[3-(5,5-dimethyl-2-oxo-pyrrolidin-3-yl)-1-phenyl-propyl]carbamate (1.55 g, 3.66 mmol) in dry tetrahydrofuran (25 mL) under reflux, 2M borane dimethyl sulfide complex in THF (15 mL, 30 mmol) was added dropwise. After 3 hours, the mixture was cooled to 0° C. and 15 mL of a solution of MeOH/HCl (9:1) were added dropwise. The mixture was stirred at 60° C. for 4 hours, allowed to cool to ambient temperature and concentrated under reduced pressure. The residue was diluted with saturated sodium bicarbonate and extracted with dichloromethane (3×50 mL). The organic layer was dried over sodium sulfate and concentrated to give crude product benzyl N-[3-(5,5-dimethylpyrrolidin-3-yl)-1-phenyl-propyl]carbamate (1.89 g, 98%) ESI-MS m/z calc. 366.2307, found 367.6 (M+1)⁺; Retention time: 3.07 minutes (LC method B).

### Step 9: tert-Butyl 4-[3-(benzyloxycarbonylamino)-3-phenyl-propyl]-2,2-dimethyl-pyrrolidine-1-carboxylate

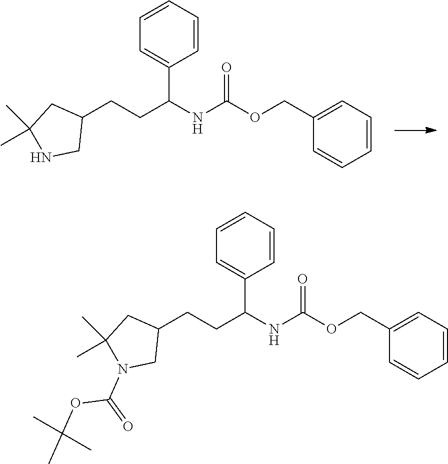 [SMILES: CC1(C)CC(CCC(NC(=O)OCc2ccccc2)c2ccccc2)CN1 ; CC(C)(C)OC(=O)N1CC(CCC(NC(=O)OCc2ccccc2)c2ccccc2)CC1(C)C]

To a solution of benzyl N-[3-(5,5-dimethylpyrrolidin-3-yl)-1-phenyl-propyl]carbamate (3.42 g, 8.3985 mmol) in TEA (2.9040 g, 4 mL, 28.698 mmol) and dichloromethane (60 mL) was added di-tert-butyl dicarbonate (4 g, 17.778 mmol) and stirred at room temperature for 1 hour. The reaction mixture was diluted with dichloromethane (300 mL), washed with sodium bicarbonate and brine, dried over sodium sulfate and concentrated. The residue was purified by silica gel column chromatography using 0-40% hexane-ethyl acetate to afford tert-butyl 4-[3-(benzyloxycarbonylamino)-3-phenyl-propyl]-2,2-dimethyl-pyrrolidine-1-carboxylate (1.7 g, 43%) ESI-MS m/z calc. 466.2832, found 467.5 (M+1)⁺; Retention time: 7.74 minutes (LC method C).

### Step 10: tert-Butyl 4-(3-amino-3-phenyl-propyl)-2,2-dimethyl-pyrrolidine-1-carboxylate

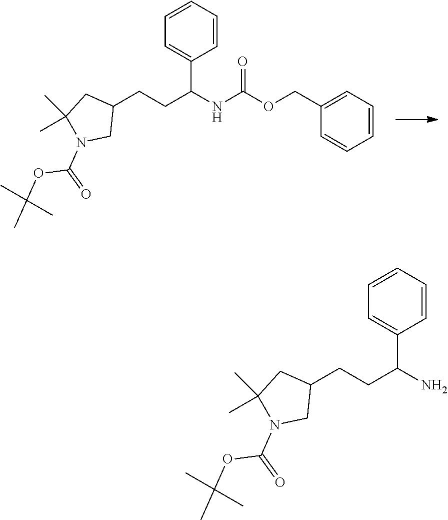 [SMILES: CC(C)(C)OC(=O)N1CC(CCC(NC(=O)OCc2ccccc2)c2ccccc2)CC1(C)C ; CC(C)(C)OC(=O)N1CC(CCC(N)c2ccccc2)CC1(C)C]

To a solution of tert-butyl 4-[3-(benzyloxycarbonylamino)-3-phenyl-propyl]-2,2-dimethyl-pyrrolidine-1-carboxylate (1.7 g, 3.57 mmol) in methanol (30 mL) and acetic acid (1 mL, 17.58 mmol) was added 10% palladium on carbon (200 mg). The mixture was in a Parr shaker under hydrogen at 55 psi for 1 hour. The reaction mixture was filtered through pad of celite. The filtrate was concentrated and diluted with ethyl acetate (100 mL) and washed with saturated sodium bicarbonate and brine. The organic layer was dried over sodium sulfate and concentrated to give tert-butyl 4-(3-amino-3-phenyl-propyl)-2,2-dimethyl-pyrrolidine-1-carboxylate (1.22 g, 98%) as a white solid. ESI-MS m/z calc. 332.2464, found 333.5 (M+1)⁺; Retention time: 3.25 minutes (LC method B).

### Step 11: tert-Butyl 2,2-dimethyl-4-[3-phenyl-3-[(6-sulfamoyl-2-pyridyl)amino]propyl]pyrrolidine-1-carboxylate

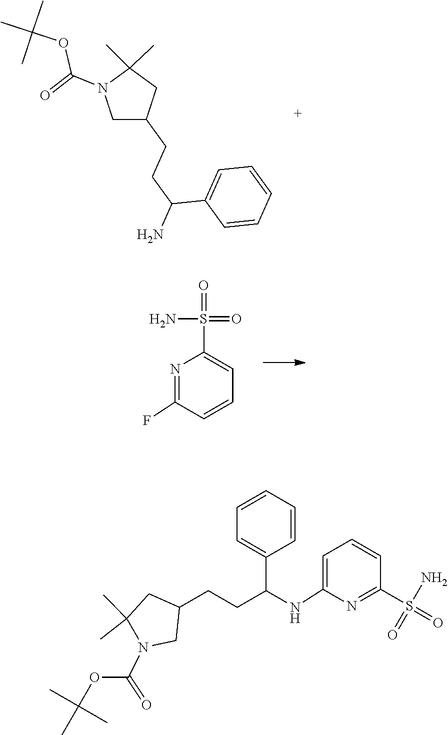 [SMILES: CC(C)(C)OC(=O)N1CC(CCC(N)c2ccccc2)CC1(C)C ; NS(=O)(=O)c1cccc(F)n1 ; CC(C)(C)OC(=O)N1CC(CCC(Nc2cccc(S(N)(=O)=O)n2)c2ccccc2)CC1(C)C]

A slurry of tert-butyl 4-(3-amino-3-phenyl-propyl)-2,2-dimethyl-pyrrolidine-1-carboxylate (1.22 g, 3.49 mmol), 6-fluoropyridine-2-sulfonamide (1 g, 5.68 mmol), and sodium carbonate (0.8 g, 7.55 mmol) in dioxane (5 mL) was heated to 110° C. for 48 hours. The mixture was cooled and water (30 mL) was added, then extracted with ethyl acetate (2×50 mL). The organic layer was dried over sodium sulfate and concentrated. The residue was purified by prep. HPLC (column: Varian C₁₈ 10 μm 5×30 cm; flow rate: 60 mL/min.; mobile phase A: water+0.1% TFA; mobile phase B: acetonitrile+0.1% TFA; method:0-45% B over 60 minutes). The combined pure fractions were mixed with saturated sodium bicarbonate to pH 8-9. Acetonitrile was removed and the aqueous phase was extracted with ethyl acetate (3×200 mL), the organic layer was washed by brine, dried over sodium sulfate and concentrated) to give tert-butyl 2,2-dimethyl-4-[3-phenyl-3-[(6-sulfamoyl-2-pyridyl)amino]propyl]pyrrolidine-1-carboxylate (1.06 g, 60%) as an off white solid. ¹H NMR (250 MHz, DMSO-d₆) δ 7.56-7.40 (m, 3H), 7.30 (t, J=7.4 Hz, 2H), 7.19 (t, J=7.2 Hz, 1H), 7.06 (s, 1H), 6.94 (d, J=7.2 Hz, 1H), 6.61 (d, J=8.5 Hz, 1H), 5.02 (s, 1H), 3.52 (s, 1H), 2.77 (t, J=10.1 Hz, 1H), 2.09 (s, 1H), 1.81 (t, J=18.2 Hz, 3H), 1.54-1.28 (m, 12H), 1.22 (s, 3H). ESI-MS m/z calc. 488.2457, found 489.4 (M+1)⁺; Retention time: 2.75 minutes (LC method H).

### Step 12: tert-Butyl 4-[3-[[6-[[2-chloro-6-[3-[2-[1-(trifluoromethyl)cyclopropyl] ethoxy]pyrazol-1-yl]pyridine-3-carbonyl]sulfamoyl]-2-pyridyl]amino]-3-phenyl-propyl]-2,2-dimethyl-pyrrolidine-1-carboxylate

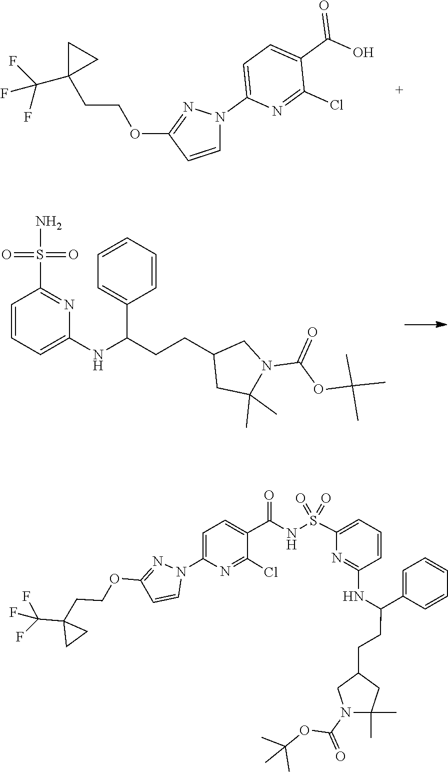 [SMILES: O=C(O)c1ccc(-n2ccc(OCCC3(C(F)(F)F)CC3)n2)nc1Cl ; CC(C)(C)OC(=O)N1CC(CCC(Nc2cccc(S(N)(=O)=O)n2)c2ccccc2)CC1(C)C ; CC(C)(C)OC(=O)N1CC(CCC(Nc2cccc(S(=O)(=O)NC(=O)c3ccc(-n4ccc(OCCC5(C(F)(F)F)CC5)n4)nc3Cl)n2)c2ccccc2)CC1(C)C]

To a solution of 2-chloro-6-[3-[2-[1-(trifluoromethyl)cyclopropyl]ethoxy]pyrazol-1-yl]pyridine-3-carboxylic acid (848 mg, 2.257 mmol) in THF (6 mL) was added CDI (457.1 mg, 2.819 mmol) (recrystallized from THF) and the mixture was stirred at rt for 1 h then tert-butyl 2,2-dimethyl-4-[3-phenyl-3-[(6-sulfamoyl-2-pyridyl)amino]propyl]pyrrolidine-1-carboxylate (1.153 g, 2.360 mmol) was added as a solution in THF (2 mL) followed by DBU (1.079 g, 7.088 mmol) and the resulting mixture was stirred for 3 h at room temperature. The reaction was diluted with water and EtOAc then HCl (2.523 mL of 6 M, 15.14 mmol) was added, aqueous layer was then pH=1. The layers were separated and the organic layer was washed with water and brine then dried over sodium sulfate and concentrated to a white foam which was purified on a 275 g C₁₈ reverse phase column eluting with a gradient from 50-100% ACN/Water giving tert-butyl 4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]-3-phenyl-propyl]-2,2-dimethyl-pyrrolidine-1-carboxylate (1.44 g, 75%) as a white solid. ESI-MS m/z calc. 845.2949, found 846.3 (M+1)⁺; Retention time: 0.91 minutes (LC method D).

### Step 13: 2-Chloro-N-[[6-[[3-(5,5-dimethylpyrrolidin-3-yl)-1-phenyl-propyl]amino]-2-pyridyl]sulfonyl]-6-[3-[2-[1-(trifluoromethyl)cyclopropyl]ethoxy]pyrazol-1-yl]pyridine-3-carboxamide

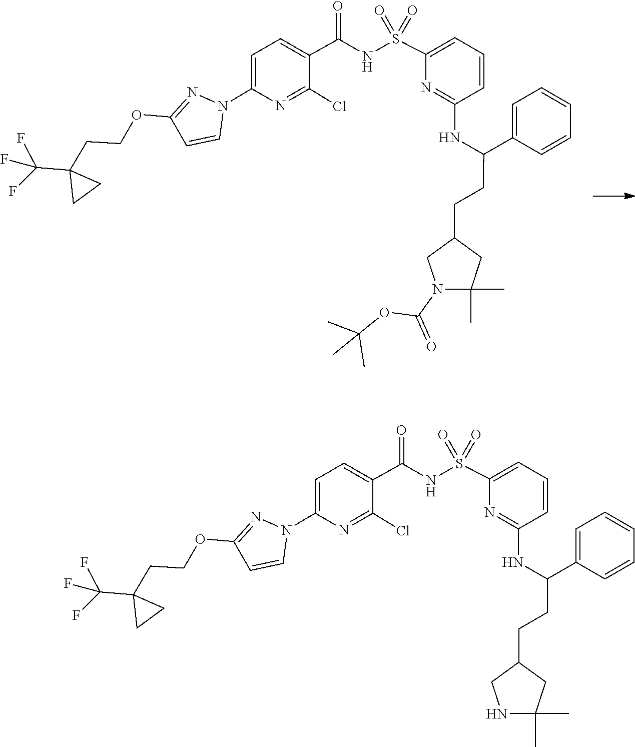 [SMILES: CC(C)(C)OC(=O)N1CC(CCC(Nc2cccc(S(=O)(=O)NC(=O)c3ccc(-n4ccc(OCCC5(C(F)(F)F)CC5)n4)nc3Cl)n2)c2ccccc2)CC1(C)C ; CC1(C)CC(CCC(Nc2cccc(S(=O)(=O)NC(=O)c3ccc(-n4ccc(OCCC5(C(F)(F)F)CC5)n4)nc3Cl)n2)c2ccccc2)CN1]

tert-Butyl 4-[3-[[6-[[2-chloro-6-[3-[2-[1-(trifluoromethyl)cyclopropyl] ethoxy]pyrazol-1-yl]pyridine-3-carbonyl]sulfamoyl]-2-pyridyl]amino]-3-phenyl-propyl]-2,2-dimethyl-pyrrolidine-1-carboxylate (1.44 g, 1.701 mmol) was dissolved in DCM (6 mL) and to the mixture was added TFA (5.2 mL, 68.08 mmol) and the mixture was stirred at room temperature for 2 h. Concentrated mixture to dryness under reduced pressure, added 50 mL of toluene and removed by rotary evaporation (45° C. water bath). Again added 50 mL of toluene and removed by rotary evaporation (45° C. water bath) then dried on the high vacuum giving 2-chloro-N-[[6-[[3-(5,5-dimethylpyrrolidin-3-yl)-1-phenyl-propyl]amino]-2-pyridyl]sulfonyl]-6-[3-[2-[1-(trifluoromethyl)cyclopropyl]ethoxy]pyrazol-1-yl]pyridine-3-carboxamide (trifluoroacetate salt) (1.463 g, 100%) ESI-MS m/z calc. 745.2425, found 746.2 (M+1)⁺; Retention time: 0.62 minutes as a white solid (LC method D).

### Step 14: 12,12-Dimethyl-17-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, diastereomer 1 and 12,12-dimethyl-17-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, diastereomer 2

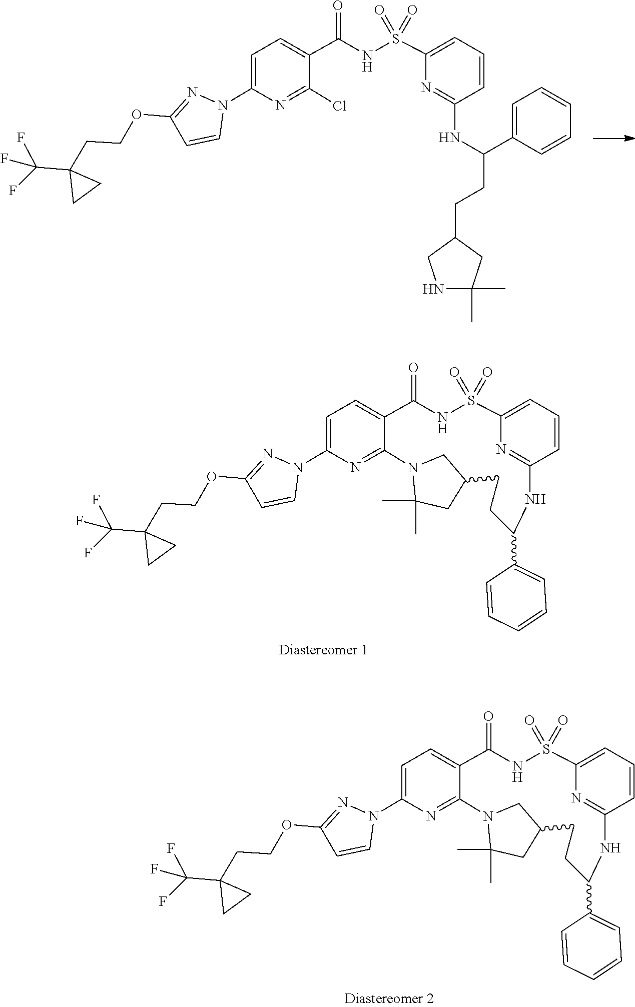 [SMILES: CC1(C)CC(CCC(Nc2cccc(S(=O)(=O)NC(=O)c3ccc(-n4ccc(OCCC5(C(F)(F)F)CC5)n4)nc3Cl)n2)c2ccccc2)CN1 ; CC1(C)CC2CCC(c3ccccc3)Nc3cccc(n3)S(=O)(=O)NC(=O)c3ccc(-n4ccc(OCCC5(C(F)(F)F)CC5)n4)nc3N1C2 ; CC1(C)CC2CCC(c3ccccc3)Nc3cccc(n3)S(=O)(=O)NC(=O)c3ccc(-n4ccc(OCCC5(C(F)(F)F)CC5)n4)nc3N1C2]

To a solution of 2-chloro-N-[[6-[[3-(5,5-dimethylpyrrolidin-3-yl)-1-phenyl-propyl]amino]-2-pyridyl]sulfonyl]-6-[3-[2-[1-(trifluoromethyl)cyclopropyl]-ethoxy]pyrazol-1-yl]pyridine-3-carboxamide (trifluoroacetate salt) (1.463 g, 1.701 mmol) in NMP (88 mL) was added K₂CO₃ (1.646 g, 11.91 mmol). The mixture was purged with nitrogen for 5 min. The mixture was heated at 150° C. overnight. The mixture was cooled to room temperature and diluted with 300 mL of water followed by the slow addition of HCl (2 mL of 12 M, 25.52 mmol) giving a precipitate. The mixture was further diluted with 100 mL of 1N aqueous HCl then extracted with EtOAc (1×300 mL) and the organic phase was washed with saturated brine. The organic layer was then dried (sodium sulfate), filtered and concentrated to a tan solid which was chromatographed on a 275 g Reverse Phase Column eluting with a gradient from 50% to 100% Acetonitrile in water giving 2 peaks corresponding to the diastereomeric products:

Diastereomer 1: 12,12-dimethyl-17-phenyl-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (211 mg, 35%) ESI-MS m/z calc. 709.2658, found 710.2 (M+1)⁺; Retention time: 0.86 minutes (LC method T).

Diastereomer 2: 12,12-dimethyl-17-phenyl-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (386 mg, 64%) ESI-MS m/z calc. 709.2658, found 710.2 (M+1)⁺; Retention time: 0.88 minutes (LC method T).

### Step 15: 12,12-Dimethyl-17-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione Compound 388 (enantiomer 1) and 12,12-Dimethyl-17-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione Compound 387 (enantiomer 2)

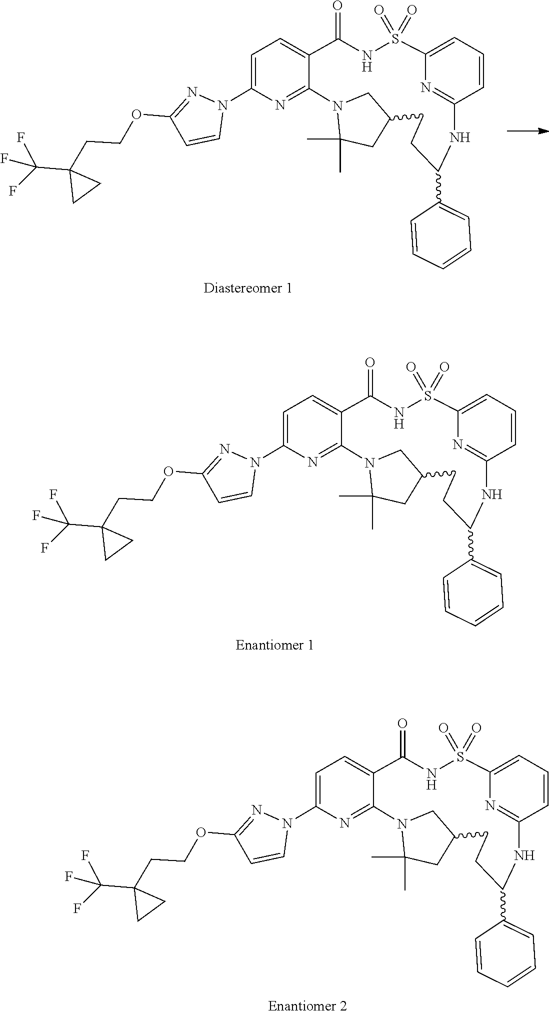 [SMILES: CC1(C)CC2CCC(c3ccccc3)Nc3cccc(n3)S(=O)(=O)NC(=O)c3ccc(-n4ccc(OCCC5(C(F)(F)F)CC5)n4)nc3N1C2 ; CC1(C)CC2CCC(c3ccccc3)Nc3cccc(n3)S(=O)(=O)NC(=O)c3ccc(-n4ccc(OCCC5(C(F)(F)F)CC5)n4)nc3N1C2 ; CC1(C)CC2CCC(c3ccccc3)Nc3cccc(n3)S(=O)(=O)NC(=O)c3ccc(-n4ccc(OCCC5(C(F)(F)F)CC5)n4)nc3N1C2]

12,12-Dimethyl-17-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (211 mg, 0.2973 mmol, (diastereomer 1) was subjected to chiral separation by SFC chromatography using ChiralPak AS-H (250×21.2 mm), 5 nm particle size using 15% MeOH (No modifier), 45% CO₂ at 10 mL/min over 14 minutes giving as white solids:

First enantiomer to elute (enantiomer 1) 12,12-dimethyl-17-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (92.2 mg, 87%). ¹H NMR (400 MHz, Chloroform-d) δ 8.20 (d, J=2.8 Hz, 1H), 8.00 (d, J=8.3 Hz, 1H), 7.57 (dd, J=8.4, 7.2 Hz, 1H), 7.49 (dd, J=7.3, 0.8 Hz, 1H), 7.39 (dt, J=6.3, 1.5 Hz, 2H), 7.36-7.31 (m, 2H), 7.30-7.24 (m, 1H), 7.18 (d, J=8.3 Hz, 1H), 6.58 (dd, J=8.3, 0.8 Hz, 1H), 5.90 (d, J=2.8 Hz, 1H), 5.40-5.31 (m, 1H), 4.75 (d, J=8.8 Hz, 1H), 4.40 (t, J=7.1 Hz, 2H), 3.44 (dd, J=10.1, 6.9 Hz, 1H), 3.03 (t, J=10.4 Hz, 1H), 2.62 (dd, J=19.5, 8.0 Hz, 1H), 2.10 (t, J=7.2 Hz, 2H), 2.04 (d, J=7.4 Hz, 1H), 2.01-1.95 (m, 1H), 1.88 (dt, J=14.8, 8.4 Hz, 1H), 1.64 (m, 7H) 1.58-1.49 (m, 1H), 1.25 (s, 1H), 1.05-1.00 (m, 2H), 0.78-0.71 (m, 2H) ESI-MS m/z calc. 709.2658, found 710.2 (M+1)⁺; Retention time: 1.3 minutes (LC method M).

Second enantiomer to elute (enantiomer 2): 12,12-dimethyl-17-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (96.8 mg, 92%). ¹H NMR (400 MHz, Chloroform-d) δ 8.20 (d, J=2.8 Hz, 1H), 8.00 (d, J=8.3 Hz, 1H), 7.57 (dd, J=8.3, 7.3 Hz, 1H), 7.49 (d, J=6.8 Hz, 1H), 7.41-7.37 (m, 2H), 7.37-7.31 (m, 2H), 7.30-7.26 (m, 1H), 7.18 (d, J=8.3 Hz, 1H), 6.58 (dd, J=8.4, 0.8 Hz, 1H), 5.90 (d, J=2.8 Hz, 1H), 5.41-5.30 (m, 1H), 4.75 (d, J=8.8 Hz, 1H), 4.40 (t, J=7.1 Hz, 2H), 3.44 (dd, J=10.1, 6.9 Hz, 1H), 3.03 (t, J=10.4 Hz, 1H), 2.62 (dd, J=19.2, 8.1 Hz, 1H), 2.11 (d, J=7.1 Hz, 2H), 2.03 (s, 1H), 2.01-1.94 (m, 1H), 1.88 (dt, J=14.9, 8.4 Hz, 1H), 1.65 (s, 3H), 1.63 (s, 3H), 1.59-1.45 (m, 2H), 1.25 (s, 2H), 1.08-0.99 (m, 2H), 0.79-0.69 (m, 2H) ESI-MS m/z calc. 709.2658, found 710.14 (M+1)⁺; Retention time: 1.29 minutes (LC method M).

### Step 16: 12,12-Dimethyl-17-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 384 (enantiomer 3), and 12,12-Dimethyl-17-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 383 (enantiomer 4)

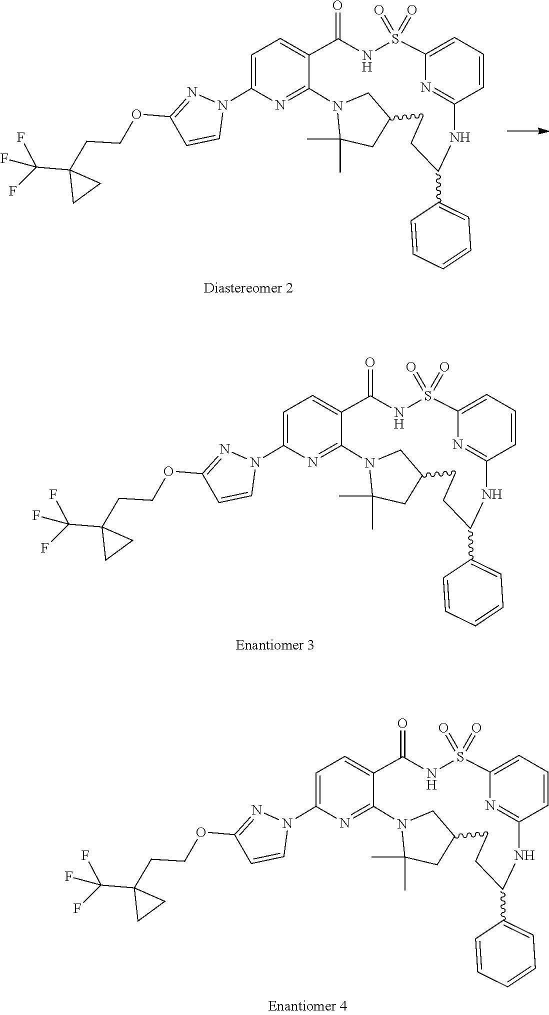 [SMILES: CC1(C)CC2CCC(c3ccccc3)Nc3cccc(n3)S(=O)(=O)NC(=O)c3ccc(-n4ccc(OCCC5(C(F)(F)F)CC5)n4)nc3N1C2 ; CC1(C)CC2CCC(c3ccccc3)Nc3cccc(n3)S(=O)(=O)NC(=O)c3ccc(-n4ccc(OCCC5(C(F)(F)F)CC5)n4)nc3N1C2 ; CC1(C)CC2CCC(c3ccccc3)Nc3cccc(n3)S(=O)(=O)NC(=O)c3ccc(-n4ccc(OCCC5(C(F)(F)F)CC5)n4)nc3N1C2]

12,12-Dimethyl-17-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (380 mg, 0.53 mmol, diastereomer 2) was subjected to chiral separation by SFC chromatography using ChiralPak AD-H (250×21.2 mm), 5 nm particle size using 15% MeOH (No modifier), 45% CO₂ at 10 mL/min over 14 minutes giving as white solids:

First enantiomer to elute (enantiomer 3): 12,12-dimethyl-17-phenyl-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (97 mg, 51%) ESI-MS m/z calc. 709.2658, found 710.14 (M+1)⁺; Retention time: 1.38 minutes (LC method M).

Second enantiomer to elute (enantiomer 4): 12,12-dimethyl-17-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (90 mg, 47%) ESI-MS m/z calc. 709.2658, found 710.2 (M+1)⁺; Retention time: 1.39 minutes (LC method M).

### Example 124: Preparation of (14S)-8-13-(2-{dispiro[2.0.2.11heptan-7-yl}ethoxy)-1H-pyrazol-1-yl]-12,12-dimethyl-17-phenyl-2λ⁶-thia-3,9,11,18,23-pentaazatetracyclo [17.3.1.111,14.05,10]tetracosa-1(22),5,7,9,19(23),20-hexaene-2,2,4-trione (diastereomer 1, Compound 364) and (14S)-8-13-(2-{dispiro[2.0.2.11heptan-7-yl}ethoxy)-1H-pyrazol-1-yl]-12,12-dimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione (diastereomer 2, Compound 363)

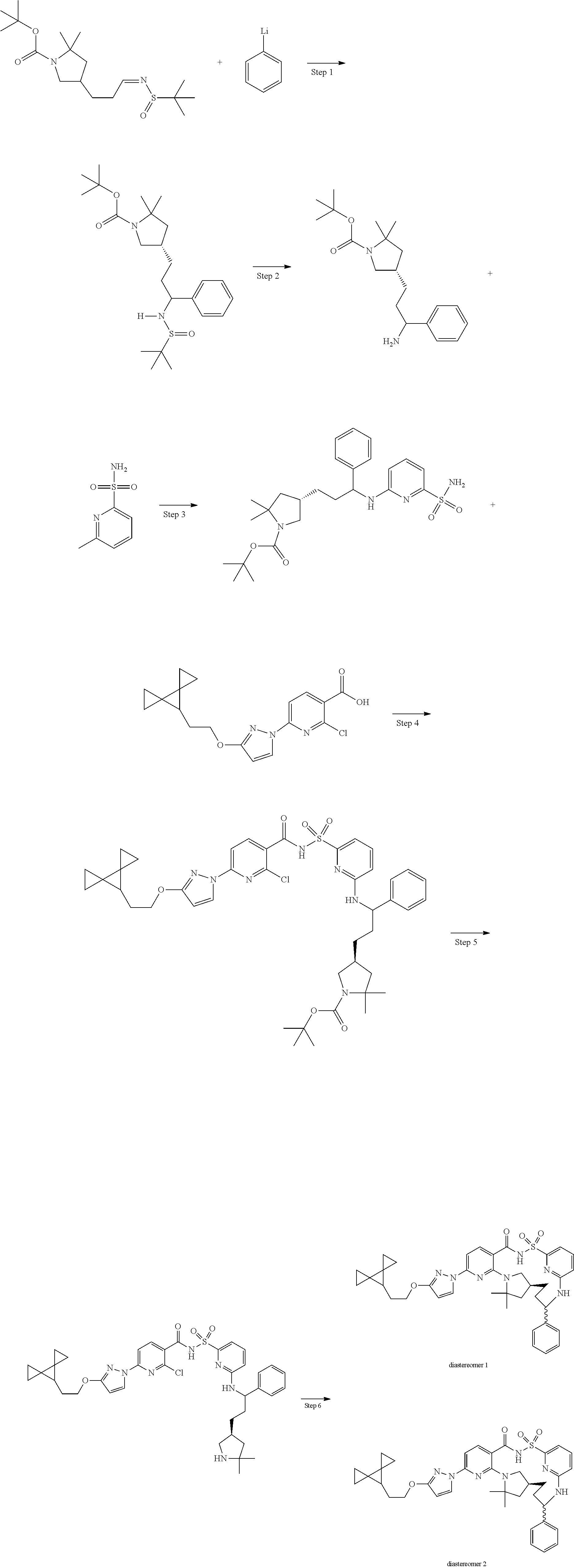

### Step 1: tert-Butyl (4S)-4-[3-(tert-butylsulfinylamino)-3-phenyl-propyl]-2,2-dimethyl-pyrrolidine-1-carboxylate

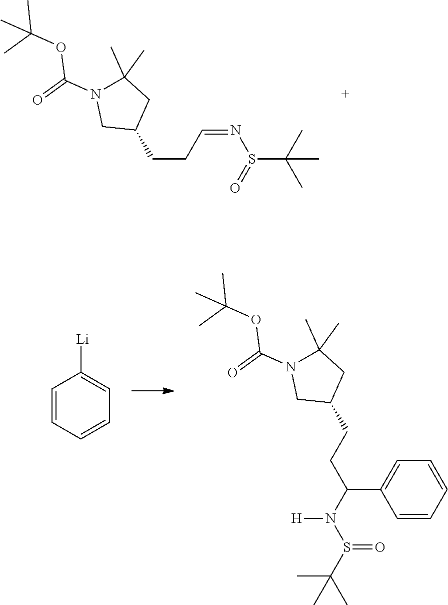

tert-Butyl (4S)-4[(3Z)-3-tert-butylsulfinyliminopropyl]-2,2-dimethyl-pyrrolidine-1-carboxylate (3.5 g, 9.2737 mmol) was dissolved in THF (50 mL) and cooled in a −70° C. dry ice acetone bath under nitrogen balloon. After 10 min, phenyllithium (5.2 mL of 1.9 M in dibutyl ether, 9.88 mmol) was added at a constant rate over 5 min. The mixture was stirred at the same temperature for 30 min and warmed up to −30° C. Saturated aqueous NH₄Cl (20 mL) was added. The mixture was warmed up to rt and partitioned between water (50 mL) and EtOAc (50 mL). The layers were separated. The EtOAc layer was washed with brine, dried over anhydrous Na₂SO₄, filtered and concentrated. The residue was purified by silica gel chromatography, using 0-40% EtOAc in hexanes to afford tert-butyl (4S)-4-[3-(tert-butylsulfinylamino)-3-phenyl-propyl]-2,2-dimethyl-pyrrolidine-1-carboxylate (3.54 g, 83%). ESI-MS m/z calc. 436.276, found 437.6 (M+1)⁺; Retention time: 3.76 minutes (LC method B).

### Step 2: tert-Butyl (4S)-4-(3-amino-3-phenyl-propyl)-2,2-dimethyl-pyrrolidine-1-carboxylate

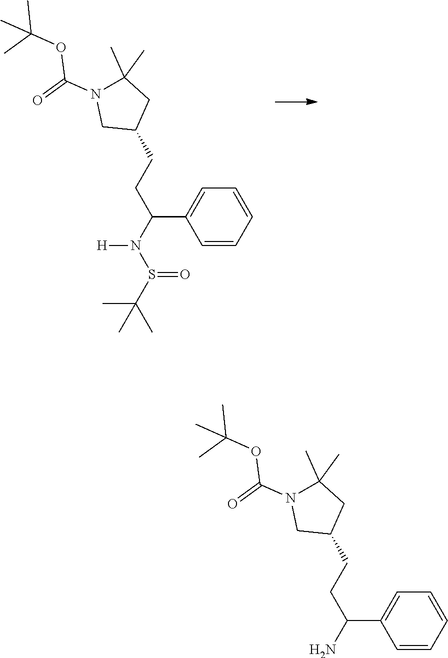 [SMILES: CC(C)(C)OC(=O)N1C[C@@H](CCC(NS(=O)C(C)(C)C)c2ccccc2)CC1(C)C ; CC(C)(C)OC(=O)N1C[C@@H](CCC(N)c2ccccc2)CC1(C)C]

tert-Butyl (4S)-4-[3-(tert-butylsulfinylamino)-3-phenyl-propyl]-2,2-dimethyl-pyrrolidine-1-carboxylate (3.54 g, 7.7018 mmol) was dissolved in a solvent mixture of THF (50 mL) and water (10 mL). Molecular iodine (391 mg, 0.0793 mL, 1.54 mmol) was added in one portion. The mixture was heated in a 50-55° C. oil bath under nitrogen for 6 h. It was then cooled to rt and diluted with EtOAc (50 mL) and aqueous NaS₂O₃ (10 g) in saturated aqueous NaHCO₃(50 mL). The layers were separated and the organic layer was washed with water (30 mL) and brine. The layers were separated and the organic layer was dried over anhydrous Na₂SO₄, filtered and concentrated. This crude material was used in the next step without further purification. ESI-MS m/z calc. 332.24637, found 333.6 (M+1)⁺; Retention time: 2.69 minutes (LC method B).

### Step 3: tert-Butyl (4S)-2,2-dimethyl-4-[3-phenyl-3-[(6-sulfamoyl-2-pyridyl)amino]propyl]pyrrolidine-1-carboxylate

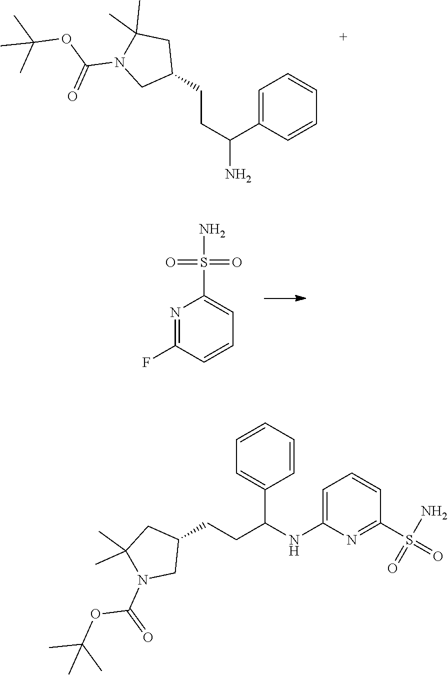 [SMILES: CC(C)(C)OC(=O)N1C[C@@H](CCC(N)c2ccccc2)CC1(C)C ; NS(=O)(=O)c1cccc(F)n1 ; CC(C)(C)OC(=O)N1C[C@@H](CCC(Nc2cccc(S(N)(=O)=O)n2)c2ccccc2)CC1(C)C]

tert-Butyl (4S)-4-(3-amino-3-phenyl-propyl)-2,2-dimethyl-pyrrolidine-1-carboxylate (3 g, 8.12 mmol) was dissolved in DMSO (5 mL). 6-Fluoropyridine-2-sulfonamide (2.1 g, 11.92 mmol) and Na₂CO₃ (2.6 g, 24.53 mmol) were added. The mixture was heated under a nitrogen balloon in a 110° C. oil bath for 18h. It was then cooled to rt and partitioned between water and EtOAc (20 mL each). The layers were separated and the aqueous layer was extracted one more time with EtOAc (20 mL). The combined EtOAc solution was concentrated and the residue was purified by silica gel chromatography, using 5-100% EtOAc in hexanes to afford tert-butyl (4S)-2,2-dimethyl-4-[3-phenyl-3-[(6-sulfamoyl-2-pyridyl)amino]propyl]pyrrolidine-1-carboxylate as a white foam (2.35 g, 57%). ¹H NMR (500 MHz, DMSO-d₆) δ 7.52-7.40 (m, 3H), 7.28 (t, J=7.6 Hz, 2H), 7.18 (t, J=7.3 Hz, 1H), 7.04 (s, 2H), 6.93 (d, J=7.2 Hz, 1H), 6.60 (d, J=8.3 Hz, 1H), 3.61-3.45 (m, 1H), 2.84-2.66 (m, 1H), 2.14-2.03 (m, 1H), 1.98 (s, 1H), 1.91-1.61 (m, 3H), 1.47-1.28 (m, 13H), 1.21 (s, 4H). ESI-MS m/z calc. 488.2457, found 489.4 (M+1)⁺; Retention time: 2.79 minutes (LC method H).

### Step 4: tert-Butyl (4S)-4-[3-[[6-[[2-chloro-6-[3-(2-dispiro[2.0.2.1]heptan-7-ylethoxy)pyrazol-1-yl]pyridine-3-carbonyl]sulfamoyl]-2-pyridyl]amino]-3-phenyl-propyl]-2,2-dimethyl-pyrrolidine-1-carboxylate

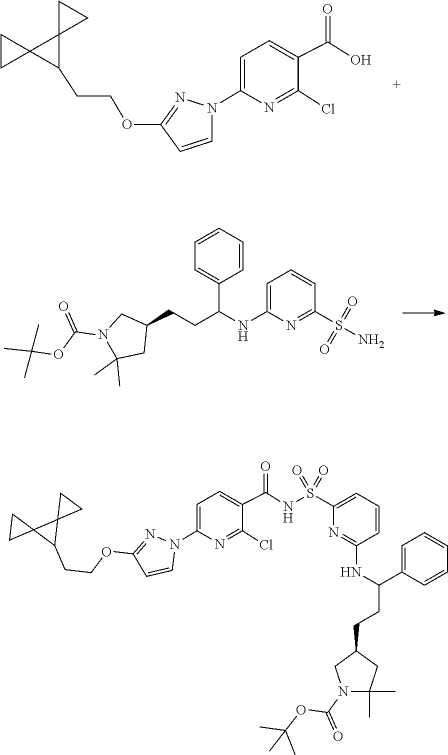 [SMILES: O=C(O)c1ccc(-n2ccc(OCCC3C4(CC4)C34CC4)n2)nc1Cl ; CC(C)(C)OC(=O)N1C[C@@H](CCC(Nc2cccc(S(N)(=O)=O)n2)c2ccccc2)CC1(C)C ; CC(C)(C)OC(=O)N1C[C@@H](CCC(Nc2cccc(S(=O)(=O)NC(=O)c3ccc(-n4ccc(OCCC5C6(CC6)C56CC6)n4)nc3Cl)n2)c2ccccc2)CC1(C)C]

To a solution of 2-Chloro-6-[3-(2-dispiro[2.0.2.1]heptan-7-ylethoxy)pyrazol-1-yl]pyridine-3-carboxylic acid (250 mg, 0.695 mmol) in THF (2 mL) was added CDI (140.7 mg, 0.868 mmol) (recrystallized from THF) and the mixture was stirred at rt for 1 h then tert-butyl (4S)-2,2-dimethyl-4-[3-phenyl-3-[(6-sulfamoyl-2-pyridyl)amino]propyl]pyrrolidine-1-carboxylate (355.1 mg, 0.727 mmol) was added followed by DBU (326.3 μL, 2.182 mmol) and the resulting mixture was stirred for 16 h at room temperature. Concentrated to remove the THF then diluted with DMSO, filtered and purified using a reverse phase HPLC-MS method using a LuNa C₁₈ (2) column (75×30 mm, 5 μm particle size) sold by Phenomenex (pn: 00C-4252-U0-AX), and a dual gradient run from 1-99% mobile phase B over 15.0 minutes (mobile phase A=H₂O (5 mM HCl), mobile phase B=acetonitrile, flow rate=50 mL/min, injection volume=950 μL and column temperature=25° C.) giving tert-butyl (4S)-4-[3-[[6-[[2-chloro-6-[3-(2-dispiro[2.0.2.1]heptan-7-ylethoxy)pyrazol-1-yl]pyridine-3-carbonyl]sulfamoyl]-2-pyridyl]amino]-3-phenyl-propyl]-2,2-dimethyl-pyrrolidine-1-carboxylate (282.6 mg, 49%) as a mixture of diastereomers. ESI-MS m/z calc. 829.3388, found 830.3 (M+1)⁺; Retention time: 0.96 minutes (LC method D).

### Step 5: 2-Chloro-N-[[6-[[3-[(3S)-5,5-dimethylpyrrolidin-3-yl]-1-phenyl-propyl]amino]-2-pyridyl]sulfonyl]-6-[3-(2-dispiro[2.0.2.1]heptan-7-ylethoxy)pyrazol-1-yl]pyridine-3-carboxamide

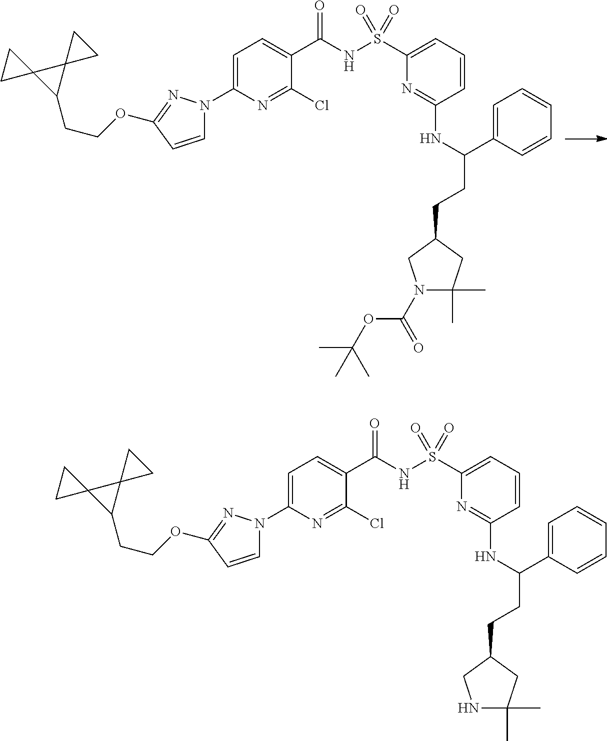 [SMILES: CC(C)(C)OC(=O)N1C[C@@H](CCC(Nc2cccc(S(=O)(=O)NC(=O)c3ccc(-n4ccc(OCCC5C6(CC6)C56CC6)n4)nc3Cl)n2)c2ccccc2)CC1(C)C ; CC1(C)C[C@H](CCC(Nc2cccc(S(=O)(=O)NC(=O)c3ccc(-n4ccc(OCCC5C6(CC6)C56CC6)n4)nc3Cl)n2)c2ccccc2)CN1]

tert-Butyl (4S)-4-[3-[[6-[[2-chloro-6-[3-(2-dispiro[2.0.2.1]heptan-7-ylethoxy)pyrazol-1-yl]pyridine-3-carbonyl]sulfamoyl]-2-pyridyl]amino]-3-phenyl-propyl]-2,2-dimethyl-pyrrolidine-1-carboxylate (282.6 mg, 0.3403 mmol) was dissolved in DCM (1.2 mL) and to the mixture was added TFA (1 mL, 13.62 mmol) and the mixture was stirred at room temperature for 2 h. Concentrated mixture to dryness under reduced pressure, added 1 mL of toluene and removed by rotary evaporation (45° C. water bath). Again added 1 mL of toluene and removed by rotary evaporation (45° C. water bath) then dried on the high vacuum giving 2-chloro-N-[[6-[[3-[(3S)-5,5-dimethylpyrrolidin-3-yl]-1-phenyl-propyl]amino]-2-pyridyl]sulfonyl]-6-[3-(2-dispiro[2.0.2.1]heptan-7-ylethoxy)pyrazol-1-yl]pyridine-3-carboxamide (trifluoroacetate salt) (287.3 mg, 100%) as a white solid. ESI-MS m/z calc. 729.2864, found 730.6 (M+1)⁺; Retention time: 0.73 minutes (LC method D).

### Step 6: (14S)-8-13-(2-{dispiro[2.0.2.11heptan-7-yl}ethoxy)-1H-pyrazol-1-yl]-12,12-dimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione (diastereomer 1, Compound 364) and (14S)-8-[3-(2-{dispiro[2.0.2.1]heptan-7-yl}ethoxy)-1H-pyrazol-1-yl]-12,12-dimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione (diastereomer 2, Compound 363)

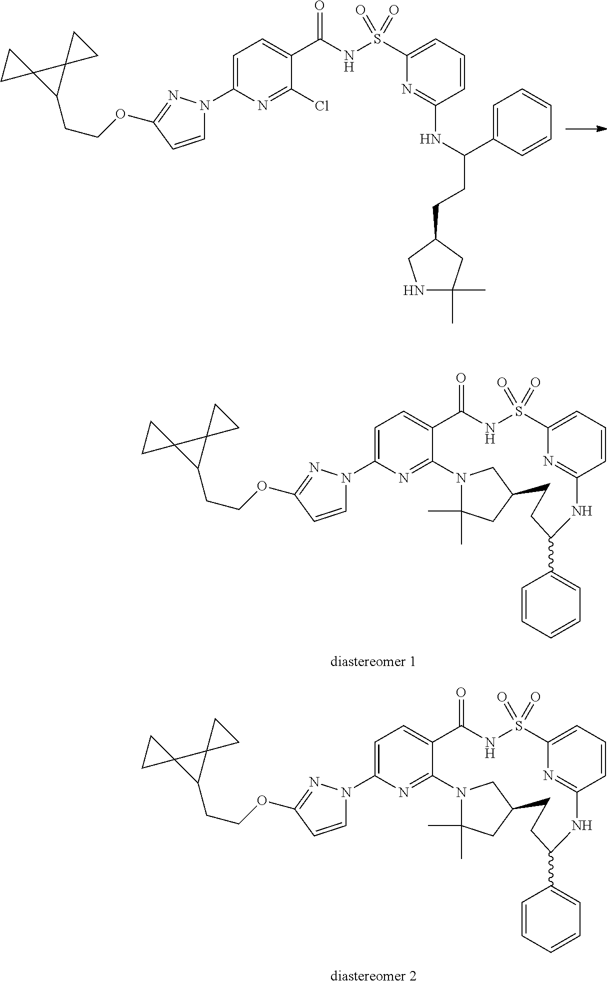 [SMILES: CC1(C)C[C@H](CCC(Nc2cccc(S(=O)(=O)NC(=O)c3ccc(-n4ccc(OCCC5C6(CC6)C56CC6)n4)nc3Cl)n2)c2ccccc2)CN1 ; CC1(C)C[C@@H]2CCC(c3ccccc3)Nc3cccc(n3)S(=O)(=O)NC(=O)c3ccc(-n4ccc(OCCC5C6(CC6)C56CC6)n4)nc3N1C2 ; CC1(C)C[C@@H]2CCC(c3ccccc3)Nc3cccc(n3)S(=O)(=O)NC(=O)c3ccc(-n4ccc(OCCC5C6(CC6)C56CC6)n4)nc3N1C2]

To a solution of 2-chloro-N-[[6-[[3-[(3S)-5,5-dimethylpyrrolidin-3-yl]-1-phenyl-propyl]amino]-2-pyridyl]sulfonyl]-6-[3-(2-dispiro[2.0.2.1]heptan-7-ylethoxy)pyrazol-1-yl]pyridine-3-carboxamide (trifluoroacetate salt) (287.3 mg, 0.3403 mmol) in NMP (17 mL) was added potassium carbonate (329.3 mg, 2.383 mmol). The mixture was purged with nitrogen for 5 min. The mixture was heated at 165° C. for 16h. The mixture was cooled to room temperature, diluted with 1N HCl and extracted with EtOAc (1×). The organic phase was washed with brine (1×), dried (sodium sulfate), filtered and concentrated to a brown oil which was filtered and purified using a reverse phase HPLC-MS method using a LuNa C₁₈ (2) column (75×30 mm, 5 μm particle size) sold by Phenomenex (pn: 00C-4252-U0-AX), and a dual gradient run from 50-99% mobile phase B over 15.0 minutes (mobile phase A=H₂O (5 mM HCl), mobile phase B=acetonitrile, flow rate=50 mL/min, injection volume=950 μL and column temperature=25° C.) giving two diastereomers as white solids:

First to elute, diastereomer 1: (14S)-8-[3-(2-{dispiro[2.0.2.1]heptan-7-yl}ethoxy)-1H-pyrazol-1-yl]-12,12-dimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione (66.5 mg, 56%). ¹H NMR (500 MHz, DMSO-d₆) δ 12.58 (s, 1H), 8.23 (d, J=2.7 Hz, 1H), 7.87 (d, J=8.2 Hz, 1H), 7.68 (d, J=9.2 Hz, 1H), 7.62 (t, J=7.9 Hz, 1H), 7.43 (d, J=7.7 Hz, 2H), 7.31 (t, J=7.5 Hz, 2H), 7.21 (t, J=7.4 Hz, 1H), 7.08 (d, J=7.2 Hz, 1H), 6.95 (d, J=8.2 Hz, 1H), 6.81 (d, J=8.5 Hz, 1H), 6.10 (d, J=2.7 Hz, 1H), 5.20 (t, J=11.1 Hz, 1H), 4.23 (t, J=6.7 Hz, 2H), 3.31 (d, J=9.6 Hz, 1H), 2.82 (t, J=10.5 Hz, 1H), 2.29 (s, 1H), 1.99-1.86 (m, 2H), 1.82 (q, J=6.6 Hz, 2H), 1.74 (dd, J=14.0, 8.7 Hz, 2H), 1.65 (s, 3H), 1.61 (d, J=12.5 Hz, 1H), 1.54 (s, 3H), 1.51-1.44 (m, 2H), 0.85 (d, J 6.2 Hz, 4H), 0.65 (dd, J=8.6, 4.2 Hz, 2H), 0.51 (d, J=8.7 Hz, 2H). ESI-MS m/z calc. 693.30975, found 694.6 (M+1)⁺; Retention time: 2.53 minutes (LC method Q), ESI-MS m/z calc. 693.30975, found 694.6 (M+1)⁺; Retention time: 1.62 minutes (LC method V, orthogonal method).

Second to elute, diastereomer 2: (14S)-8-[3-(2-{dispiro[2.0.2.1]heptan-7-yl}ethoxy)-1H-pyrazol-1-yl]-12,12-dimethyl-17-phenyl-2λ⁶-thia-3,9,11,18,23-pentaazatetracyclo[17.3.1.111,14.05,10]tetracosa-1(22),5,7,9,19(23),20-hexaene-2,2,4-trione (62.12 mg, 53%). ¹H NMR (500 MHz, DMSO-d₆) δ 12.31 (s, 1H), 8.22 (d, J=2.7 Hz, 1H), 7.76 (s, 1H), 7.67 (d, J=8.3 Hz, 1H), 7.61 (t, J=7.9 Hz, 1H), 7.52 (d, J=7.5 Hz, 2H), 7.35 (s, 2H), 7.26 (d, J=7.5 Hz, 1H), 7.18 (d, J=7.2 Hz, 1H), 6.93 (d, J=8.3 Hz, 1H), 6.77 (d, J=8.5 Hz, 1H), 6.10 (d, J=2.7 Hz, 1H), 4.97 (s, 1H), 4.22 (t, J=6.6 Hz, 2H), 3.21 (d, J=35.4 Hz, 2H), 2.34 (s, 1H), 2.19 (t, J=7.4 Hz, 1H), 1.96 (s, 1H), 1.90 (dd, J=12.0, 5.8 Hz, 1H), 1.82 (q, J=6.6 Hz, 2H), 1.72 (s, 1H), 1.64 (s, 3H), 1.55 (s, 4H), 1.48 (t, J=6.6 Hz, 1H), 1.15 (s, 1H), 0.89-0.79 (m, 4H), 0.65 (dd, J=8.3, 4.3 Hz, 2H), 0.51 (dd, J=8.3, 4.1 Hz, 2H). ESI-MS m/z calc. 693.30975, found 694.2 (M+1)⁺; Retention time: 2.3 minutes (LC method G), ESI-MS m/z calc. 693.30975, found 694.6 (M+1)⁺; Retention time: 1.75 minutes (LC method V, orthogonal method).

### Example 125: Preparation of 4-[(14S)-12,12-dimethyl-2,2,4-trioxo-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaen-17-yl]benzoic acid (diastereomer 1, Compound 371) and 4-[(14S)-12,12-dimethyl-2,2,4-trioxo-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaen-17-yl]benzoic acid (diastereomer 2, Compound 370)

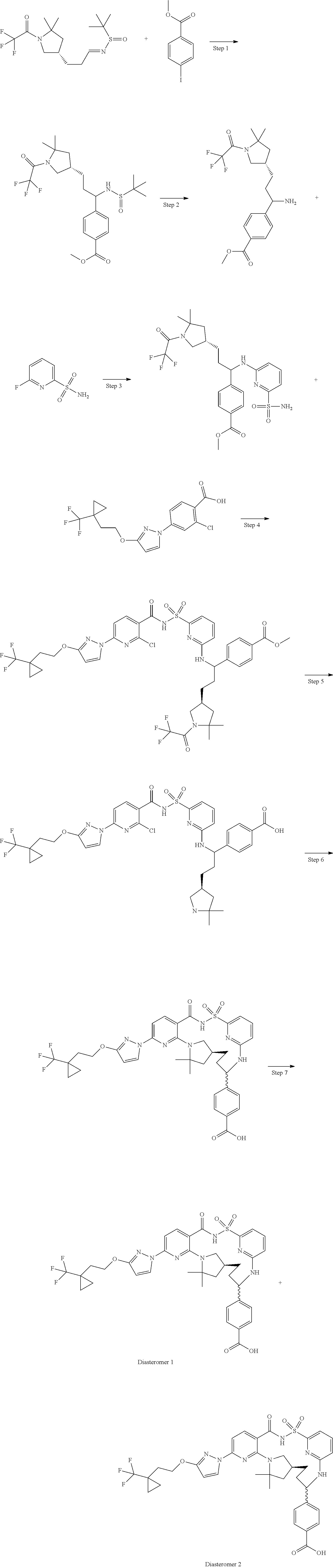

### Step 1: Methyl 4-[1-(tert-butylsulfinylamino)-3-[(3S)-5,5-dimethyl-1-(2,2,2-trifluoroacetyl)pyrrolidin-3-yl]propyl]benzoate

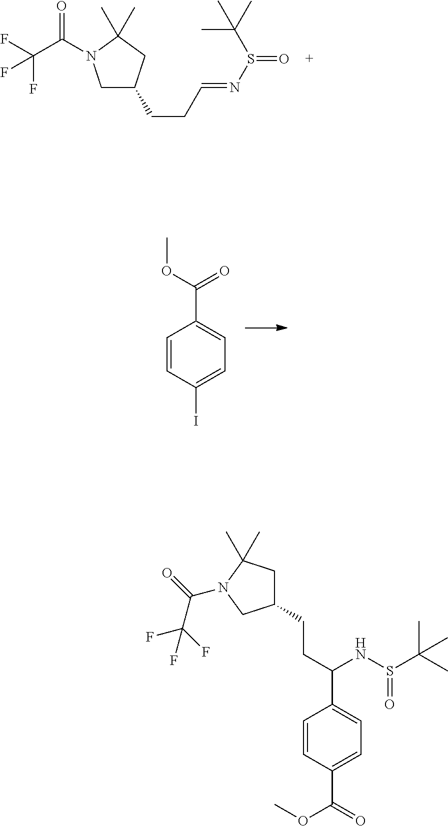 [SMILES: CC1(C)C[C@H](CC/C=N/S(=O)C(C)(C)C)CN1C(=O)C(F)(F)F ; COC(=O)c1ccc(I)cc1 ; COC(=O)c1ccc(C(CC[C@@H]2CN(C(=O)C(F)(F)F)C(C)(C)C2)NS(=O)C(C)(C)C)cc1]

Methyl 4-iodobenzoate (5.1 g, 19.073 mmol) was dissolved in THF (75 mL) and the clear solution was cooled in a dry ice/acetone bath (−23 to −19° C.) under nitrogen. Isopropyl magnesium chloride (14.8 mL of 1.3 M in THF, 19.240 mmol) was then added in portions via syringe over 3 min. The resulting mixture was stirred within the same temperature range for 100 min. It was then cooled to −30° C. A solution of N-[3-[(3S)-5,5-dimethyl-1-(2,2,2-trifluoroacetyl)pyrrolidin-3-yl]propylidene]-2-methyl-propane-2-sulfinamide (2.37 g, 6.352 mmol) in DCM (20 mL) was added slowly within 5 min. The mixture was further stirred at −30° C. for 2 h. Saturated aqueous NH₄Cl (30 mL) was added in one portion and the mixture was allowed to warm to rt outside of the cooling bath. Most volatiles were evaporated and the residue was partitioned between EtOAc and water (50 mL each). The layers were separated and the aqueous layer was extracted once with EtOAc (20 mL). The combined organics were dried over anhydrous MgSO₄, filtered and concentrated. The residue was purified by silica gel chromatography, using 5-100% EtOAc in hexanes to afford methyl 4-[1-(tert-butylsulfinylamino)-3-[(3S)-5,5-dimethyl-1-(2,2,2-trifluoroacetyl)pyrrolidin-3-yl]propyl]benzoate (2.66 g, 81%). ESI-MS m/z calc. 490.2113, found 491.6 (M+1)⁺; Retention time: 3.32 minutes (LC method B).

### Step 2: Methyl 4-[1-amino-3-[(3S)-5,5-dimethyl-1-(2,2,2-trifluoroacetyl)pyrrolidin-3-yl]propyl]benzoate

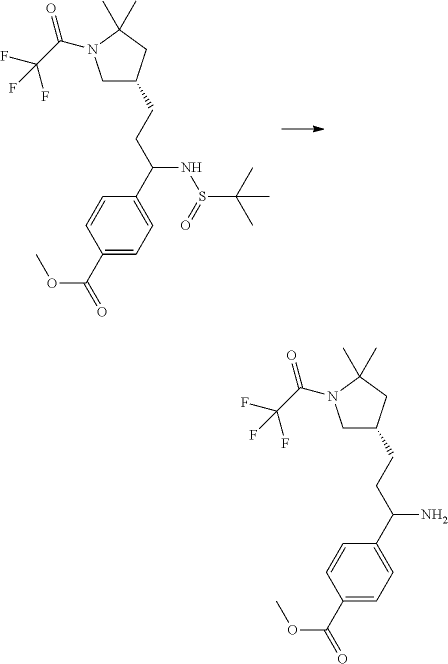 [SMILES: COC(=O)c1ccc(C(CC[C@@H]2CN(C(=O)C(F)(F)F)C(C)(C)C2)NS(=O)C(C)(C)C)cc1 ; COC(=O)c1ccc(C(N)CC[C@@H]2CN(C(=O)C(F)(F)F)C(C)(C)C2)cc1]

Methyl 4-[1-(tert-butylsulfinylamino)-3-[(3S)-5,5-dimethyl-1-(2,2,2-trifluoroacetyl)pyrrolidin-3-yl]propyl]benzoate (2.6 g, 5.035 mmol) was dissolved in MeOH (30 mL) at rt. A solution of HCl (12 mL of 4 M, 48.00 mmol) in dioxane was added via pipette. The mixture was stirred at rt for 1 h. The mixture was concentrated in vacuo to remove most volatiles. The residue was partitioned between DCM and saturated aqueous NaHCO₃(40 mL each). The layers were separated and the aqueous layer was extracted once with DCM (15 mL). The combined organics were dried over anhydrous Na₂SO₄, filtered and concentrated to afford methyl 4-[1-amino-3-[(3S)-5,5-dimethyl-1-(2,2,2-trifluoroacetyl)pyrrolidin-3-yl]propyl]benzoate (1.9 g, 93%). ESI-MS m/z calc. 386.1817, found 387.5 (M+1)⁺; Retention time: 2.43 minutes (LC method B).

### Step 3: Methyl 4-[3-[(3S)-5,5-dimethyl-1-(2,2,2-trifluoroacetyl)pyrrolidin-3-yl]-1-[(6-sulfamoyl-2-pyridyl)amino]propyl]benzoate

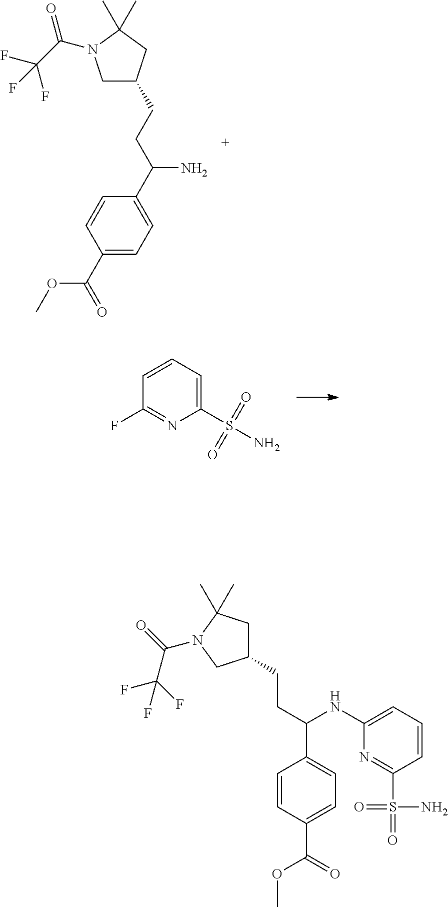 [SMILES: COC(=O)c1ccc(C(N)CC[C@@H]2CN(C(=O)C(F)(F)F)C(C)(C)C2)cc1 ; NS(=O)(=O)c1cccc(F)n1 ; COC(=O)c1ccc(C(CC[C@@H]2CN(C(=O)C(F)(F)F)C(C)(C)C2)Nc2cccc(S(N)(=O)=O)n2)cc1]

Methyl 4-[1-amino-3-[(3S)-5,5-dimethyl-1-(2,2,2-trifluoroacetyl)pyrrolidin-3-yl]propyl]benzoate (1.9 g, 4.671 mmol) was dissolved in DMSO (3 mL) at rt. 6-Fluoropyridine-2-sulfonamide (1.32 g, 7.493 mmol) was added, followed by Na₂CO₃ (1.5 g, 14.153 mmol). The mixture was heated in a 110° C. oil bath for 16 h. It was then cooled to rt, diluted with EtOAc (30 mL) and water (30 mL). The organic layer was further washed with water (20 mL×2), brine, dried over anhydrous MgSO₄, filtered and concentrated. The residue was purified by silica gel chromatography, using 20-80% EtOAc in hexanes to afford methyl 4-[3-[(3S)-5,5-dimethyl-1-(2,2,2-trifluoroacetyl)pyrrolidin-3-yl]-1-[(6-sulfamoyl-2-pyridyl)amino]propyl]benzoate (1.067 g, 40%). ¹H NMR (500 MHz, DMSO-d₆) δ 7.89 (d, J=8.2 Hz, 2H), 7.59 (d, J=8.3 Hz, 3H), 7.54-7.47 (m, 1H), 7.05 (s, 2H), 6.95 (d, J=7.2 Hz, 1H), 6.62 (d, J=8.4 Hz, 1H), 3.81 (m, 4H), 3.19-3.04 (m, 1H), 2.34-2.20 (m, 1H), 2.00-1.66 (m, 3H), 1.55-1.20 (m, 4H), 1.43 (s, 3H), 1.34-1.31 (s, 3H). ESI-MS m/z calc. 542.1811, found 543.4 (M+1)⁺; Retention time: 2.48 minutes (LC method H).

### Step 4: Methyl 4-[1-[[6-[[2-chloro-6-[3-[2-[1-(trifluoromethyl)cyclopropyl] ethoxy]pyrazol-1-yl]pyridine-3-carbonyl]sulfamoyl]-2-pyridyl]amino]-3-[(3S)-5,5-dimethyl-1-(2,2,2-trifluoroacetyl)pyrrolidin-3-yl]propyl]benzoate

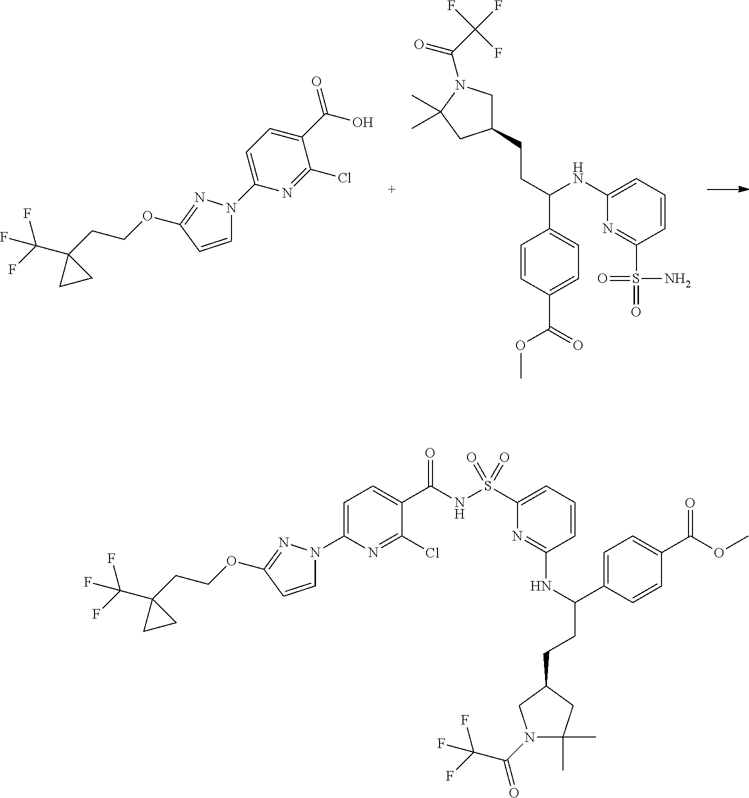 [SMILES: O=C(O)c1ccc(-n2ccc(OCCC3(C(F)(F)F)CC3)n2)nc1Cl ; COC(=O)c1ccc(C(CC[C@@H]2CN(C(=O)C(F)(F)F)C(C)(C)C2)Nc2cccc(S(N)(=O)=O)n2)cc1 ; COC(=O)c1ccc(C(CC[C@@H]2CN(C(=O)C(F)(F)F)C(C)(C)C2)Nc2cccc(S(=O)(=O)NC(=O)c3ccc(-n4ccc(OCCC5(C(F)(F)F)CC5)n4)nc3Cl)n2)cc1]

To a solution of 2-chloro-6-[3-[2-[1-(trifluoromethyl)cyclopropyl]ethoxy] pyrazol-1-yl]pyridine-3-carboxylic acid (232 mg, 0.6175 mmol) in THF (1.6 mL) was added CDI (125.1 mg, 0.771 mmol) (recrystallized from THF) and the mixture was stirred at rt for 1 h. Then methyl 4-[3-[(3S)-5,5-dimethyl-1-(2,2,2-trifluoroacetyl)pyrrolidin-3-yl]-[4(6-sulfamoyl-2-pyridyl)amino]propyl]benzoate (350.4 mg, 0.646 mmol) was added as a solution in THF (580 μL) followed by DBU (295.2 mg, 1.939 mmol) and the resulting mixture was stirred for 3 h at room temperature. The reaction was diluted with water and EtOAc then aqueous HCl (690.3 μL of 6 M, 4.142 mmol) was added (such as the aqueous layer was at pH=1). The layers were separated and the organic layer was washed with water (1×) and brine (1×) then dried over anhydrous sodium sulfate and concentrated to a white foam which was purified on a 275 g C₁₈ reverse phase column eluting with a gradient from 50-100% ACN/Water giving methyl 4-[1-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]-[3(3S)-5,5-dimethyl-1-(2,2,2-trifluoroacetyl)pyrrolidin-3-yl]propyl]benzoate (385.3 mg, 69%) as a white solid. ¹H NMR (499 MHz, DMSO-d₆) δ 12.69 (s, 1H), 8.38 (d, J=2.9 Hz, 1H), 7.96 (s, 1H), 7.81 (d, J=8.1 Hz, 2H), 7.64 (dd, J=8.3, 1.7 Hz, 1H), 7.60 (s, 1H), 7.52 (d, J=8.2 Hz, 2H), 7.15 (d, J=7.2 Hz, 1H), 6.74 (s, 1H), 6.18 (d, J=2.9 Hz, 1H), 4.99 (s, 1H), 4.35 (s, 2H), 3.78 (s, 3H), 3.76 (d, J=13.2 Hz, 1H), 3.08 (d, J=11.7 Hz, 1H), 2.25-2.16 (m, 1H), 2.09 (t, J=7.1 Hz, 2H), 1.89 (dd, J=11.5, 6.3 Hz, 1H), 1.80-1.72 (m, 1H), 1.51-1.43 (m, 1H), 1.40 (d, J=5.4 Hz, 4H), 1.29 (d, J=1.9 Hz, 3H), 1.23 (s, 3H), 0.96 (d, J=2.5 Hz, 2H), 0.89 (d, J=2.3 Hz, 2H). ESI-MS m/z calc. 899.2303, found 900.1 (M+1)⁺; Retention time: 0.84 minutes (LC method D).

### Step 5: 4-[1-[[6-[[2-chloro-6-[3-[2-[1-(Trifluoromethyl)cyclopropyl]ethoxy]pyrazol-1-yl]pyridine-3-carbonyl]sulfamoyl]-2-pyridyl]amino]-3-[(3S)-5,5-dimethylpyrrolidin-3-yl]propyl]benzoic acid

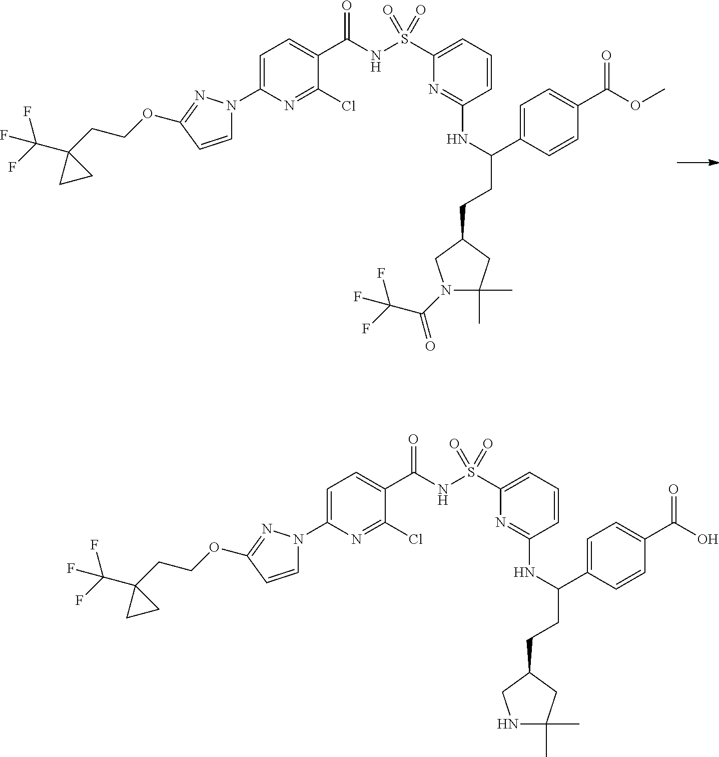 [SMILES: COC(=O)c1ccc(C(CC[C@@H]2CN(C(=O)C(F)(F)F)C(C)(C)C2)Nc2cccc(S(=O)(=O)NC(=O)c3ccc(-n4ccc(OCCC5(C(F)(F)F)CC5)n4)nc3Cl)n2)cc1 ; CC1(C)C[C@H](CCC(Nc2cccc(S(=O)(=O)NC(=O)c3ccc(-n4ccc(OCCC5(C(F)(F)F)CC5)n4)nc3Cl)n2)c2ccc(C(=O)O)cc2)CN1]

In a vial, methyl 4-[1-[[6-[[2-chloro-6-[3-[2-[1-(trifluoromethyl)cyclopropyl] ethoxy]pyrazol-1-yl]pyridine-3-carbonyl]sulfamoyl]-2-pyridyl]amino]-3-[(3S)-5,5-dimethyl-1-(2,2,2-trifluoroacetyl)pyrrolidin-3-yl]propyl]benzoate (275 mg, 0.305 mmol) and potassium carbonate (422.2 mg, 3.055 mmol) were combined with isopropanol (2.75 mL) and water (2.75 mL), capped, heated to 90° C. and stirred for 2h. Ethanol (2.75 mL) was added, then the reaction was capped, heated to 90° C. and stirred for 24h. Methanol (25 μL, 0.6110 mmol) was added, then the reaction was capped and stirred at 90° C. for 1.5h. Methanol (124 μL, 3.056 mmol) was added, then the reaction vial was heated to 100° C. and stirred for 30 min, then cooled to rt. Potassium carbonate (422 mg, 3.055 mmol) was added, then the reaction vial was capped, stirred and heated to 100° C. for 16h, then cooled to rt. Methanol (2 mL, 49.37 mmol) was added, then the reaction vial was capped, heated to 100° C. and stirred for 2 days. The mixture was then cooled to room temperature, diluted with water (100 mL) and 1N HCl (100 mL) and extracted with EtOAc (3×, added a trace amount of MeOH to each extraction for solubility). Combined the organic layers, dried over sodium sulfate, filtered and concentrated to a white solid which still contained some starting material, 4-[1-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]-3-[(3S)-5,5-dimethylpyrrolidin-3-yl]propyl]benzoic acid (hydrochloride salt) (219.6 mg, 70%) ESI-MS m/z calc. 789.2323, found 790.1 (M+1)⁺; Retention time: 0.63 minutes (LC method D).

### Step 6. 4-[(14S)-12,12-Dimethyl-2,2,4-trioxo-8-(3-{2-[1-(trifluoromethyl)cyclo propyl]ethoxy}-1H-pyrazol-1-yl)-2λ⁶-thia-3,9,11,18,23-pentaazatetracyclo [17.3.1.111,14.05,10]tetracosa-1(22),5,7,9,19(23),20-hexaen-17-yl]benzoic acid

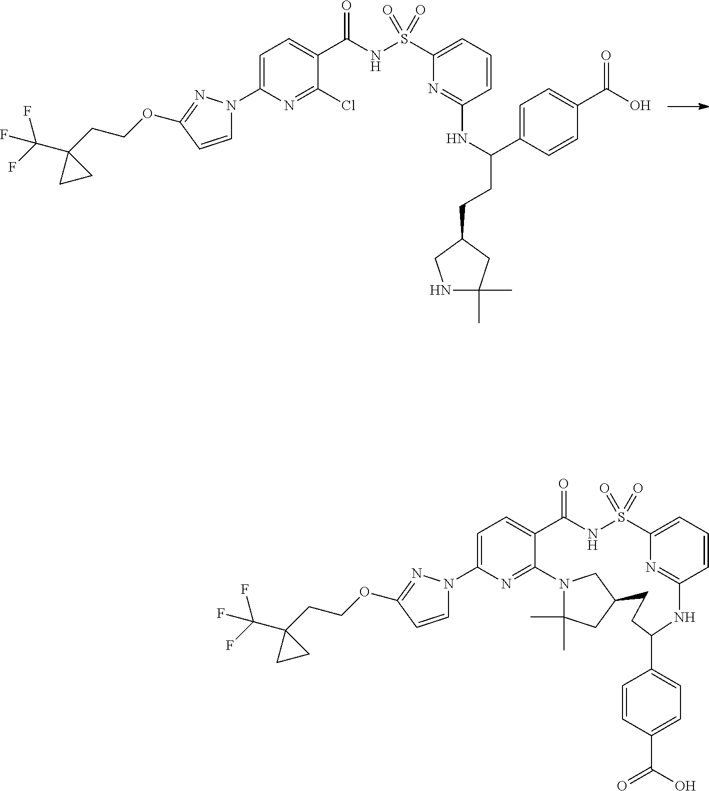 [SMILES: CC1(C)C[C@H](CCC(Nc2cccc(S(=O)(=O)NC(=O)c3ccc(-n4ccc(OCCC5(C(F)(F)F)CC5)n4)nc3Cl)n2)c2ccc(C(=O)O)cc2)CN1 ; CC1(C)C[C@@H]2CCC(c3ccc(C(=O)O)cc3)Nc3cccc(n3)S(=O)(=O)NC(=O)c3ccc(-n4ccc(OCCC5(C(F)(F)F)CC5)n4)nc3N1C2]

To a solution of 4-[1-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]-3-[(3S)-5,5-dimethylpyrrolidin-3-yl]propyl]benzoic acid (hydrochloride salt) (219.6 mg, 0.213 mmol) in NMP (11 mL) was added potassium carbonate (206.1 mg, 1.491 mmol). The mixture was purged with nitrogen for 5 min. The mixture was heated at 120° C. for 2h then heated to 150° C. and stirred for 16 h. The mixture was cooled to room temperature and diluted with 1N HCl then extracted with EtOAc (2×) and the organic phase was then dried (sodium sulfate), filtered and concentrated to a brown oil which was dissolved in DMSO, filtered and purified using a reverse phase HPLC-MS method using a LuNa C₁₈ (2) column (75×30 mm, 5 μm particle size) sold by Phenomenex (pn: 00C-4252-U0-AX), and a dual gradient run from 1-99% mobile phase B over 15.0 minutes (mobile phase A=H₂O (5 mM HCl), mobile phase B=acetonitrile, flow rate=50 mL/min, injection volume=950 μL and column temperature=25° C.) giving 4-[(14S)-12,12-dimethyl-2,2,4-trioxo-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaen-17-yl]benzoic acid (65 mg, 40%). ESI-MS m/z calc. 753.2556, found 754.1 (M+1)⁺; Retention time: 0.81 minutes as a white solid (LC method D).

### Step 7: 4-[(14S)-12,12-dimethyl-2,2,4-trioxo-8-(3-{2-[1-(trifluoromethyl)cyclopropyl] ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaen-17-yl]benzoic acid (diastereomer 1, Compound 371) and 4-[(14S)-12,12-dimethyl-2,2,4-trioxo-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaen-17-yl]benzoic acid (diastereomer 2, Compound 370)

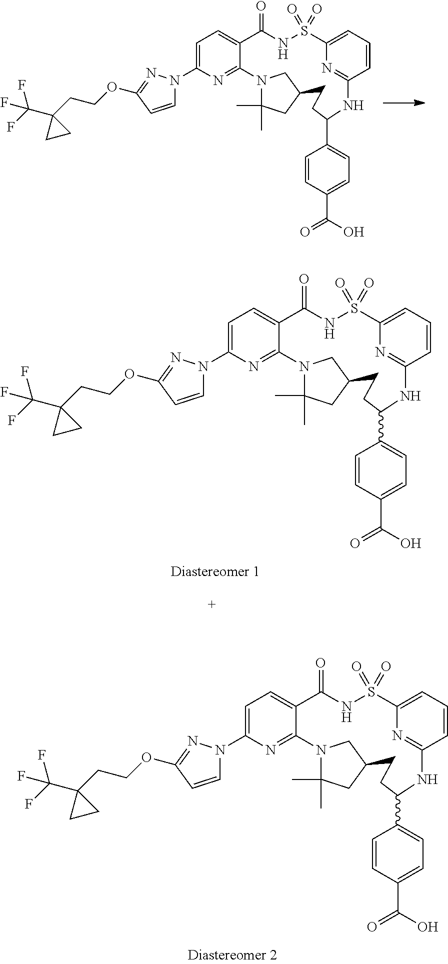 [SMILES: CC1(C)C[C@@H]2CCC(c3ccc(C(=O)O)cc3)Nc3cccc(n3)S(=O)(=O)NC(=O)c3ccc(-n4ccc(OCCC5(C(F)(F)F)CC5)n4)nc3N1C2 ; CC1(C)C[C@@H]2CCC(c3ccc(C(=O)O)cc3)Nc3cccc(n3)S(=O)(=O)NC(=O)c3ccc(-n4ccc(OCCC5(C(F)(F)F)CC5)n4)nc3N1C2 ; CC1(C)C[C@@H]2CCC(c3ccc(C(=O)O)cc3)Nc3cccc(n3)S(=O)(=O)NC(=O)c3ccc(-n4ccc(OCCC5(C(F)(F)F)CC5)n4)nc3N1C2]

Subjected 4-[(14S)-12,12-dimethyl-2,2,4-trioxo-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaen-17-yl]benzoic acid (65 mg, 0.08623 mmol) to chiral separation by SFC chromatography using a ChiralPak IG (250×21.2 mm column, 5 μm particle size) with 40% MeOH/60% CO₂ mobile phase at 70 mL/min over 11.0 minutes (injection volume=500 μL of 32 mg/mL solution in MeOH giving two diastereomers:

First to elute, diastereomer 1: 4-[(14S)-12,12-dimethyl-2,2,4-trioxo-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaen-17-yl]benzoic acid (28 mg, 86%). ¹H NMR (500 MHz, DMSO-d₆) δ 8.23 (d, J=2.7 Hz, 1H), 7.88 (d, J=8.0 Hz, 2H), 7.84 (d, J=8.1 Hz, 1H), 7.59 (t, J=7.9 Hz, 1H), 7.55 (d, J 8.1 Hz, 2H), 7.40 (s, 1H), 7.08 (d, J=7.2 Hz, 1H), 6.92 (d, J=8.2 Hz, 1H), 6.78 (d, J 8.7 Hz, 1H), 6.07 (d, J=2.8 Hz, 1H), 5.33 (d, J=11.2 Hz, 1H), 4.34 (t, J=6.9 Hz, 2H), 3.43 (s, 1H), 2.93 (s, 1H), 2.29 (s, 1H), 2.11 (t, J=7.0 Hz, 2H), 1.98 (s, 1H), 1.92-1.87 (m, 1H), 1.82-1.71 (m, 2H), 1.66 (s, 3H), 1.61 (t, J=12.3 Hz, 1H), 1.56 (s, 3H), 1.52 (d, J=12.5 Hz, 1H), 0.98 (d, J=5.1 Hz, 2H), 0.90 (s, 2H). ESI-MS m/z calc. 753.2556, found 754.6 (M+1)⁺; Retention time: 2.18 minutes (LC method Q).

Second to elute, diastereomer 2: 4-[(14S)-12,12-dimethyl-2,2,4-trioxo-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaen-17-yl]benzoic acid (18.8 mg, 55%). ¹H NMR (500 MHz, DMSO-d₆) δ 8.22 (d, J=2.8 Hz, 1H), 7.92 (d, J=8.0 Hz, 2H), 7.69 (d, J=8.1 Hz, 1H), 7.65 (d, J=8.0 Hz, 2H), 7.57 (t, J=7.9 Hz, 1H), 7.37 (s, 1H), 7.20 (d, J=7.2 Hz, 1H), 6.90 (d, J=8.1 Hz, 1H), 6.73 (d, J=8.5 Hz, 1H), 6.06 (d, J=2.7 Hz, 1H), 4.99 (s, 1H), 4.34 (t, J=7.0 Hz, 2H), 3.38 (s, 2H), 2.30 (s, 2H), 2.11 (q, J=6.9 Hz, 2H), 1.95 (s, 1H), 1.90 (dd, J=12.0, 5.9 Hz, 1H), 1.74 (s, 1H), 1.65 (s, 3H), 1.59 (d, J=11.6 Hz, 1H), 1.56 (s, 3H), 1.22-1.13 (m, 1H), 0.97 (d, J=5.2 Hz, 2H), 0.89 (d, J=9.2 Hz, 2H). ESI-MS m/z calc. 753.2556, found 754.6 (M+1)⁺; Retention time: 2.19 minutes (LC method Q).

### Example 126: Preparation of (14S)-12,12-dimethyl-17-(pyrimidin-5-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 1, Compound 375) and (14S)-12,12-dimethyl-17-(pyrimidin-5-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 2, Compound 374)

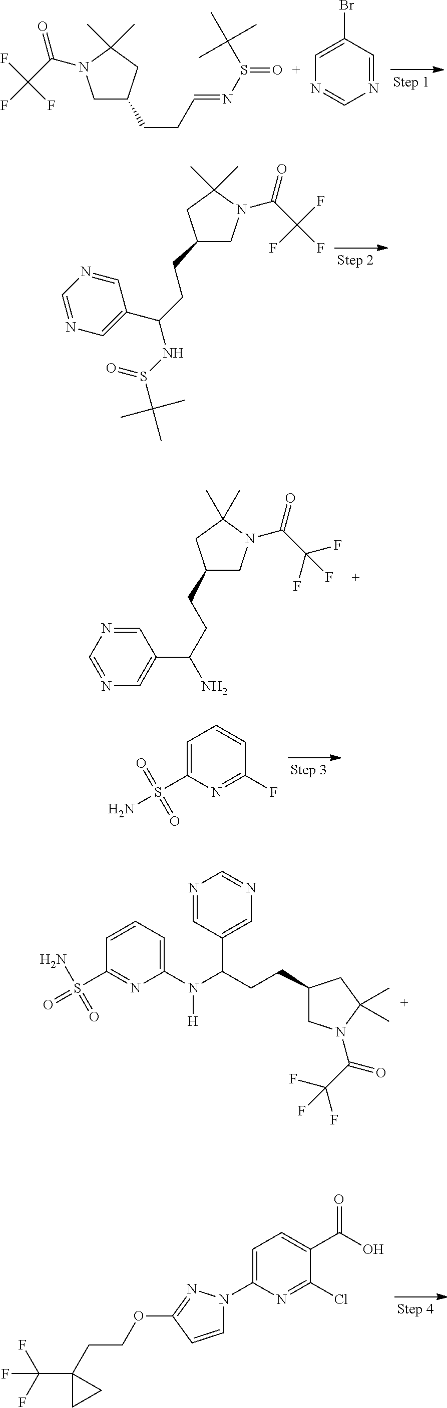

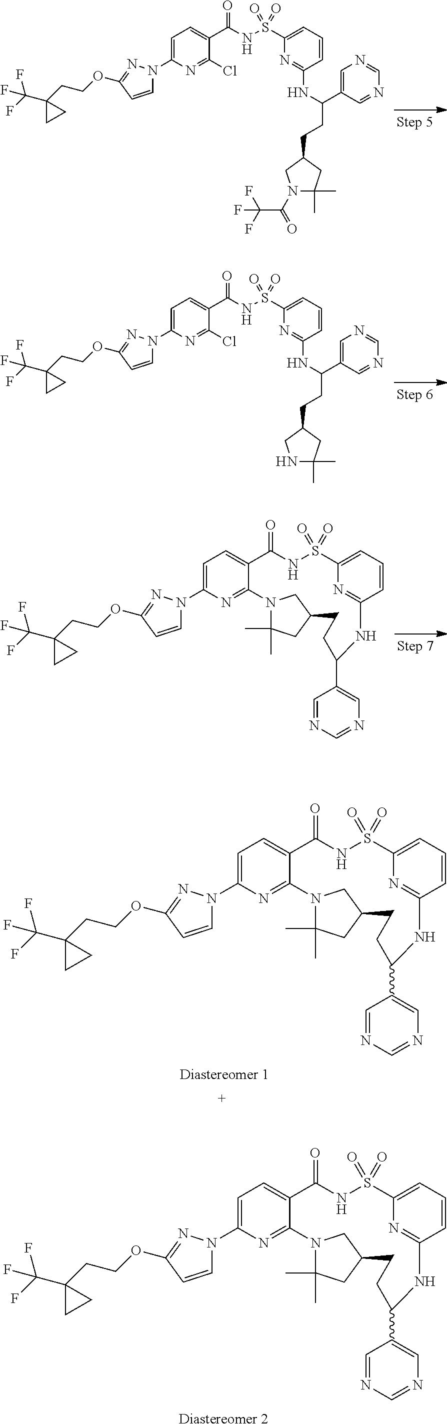

### Step 1: N-[3-[(3S)-5,5-Dimethyl-1-(2,2,2-trifluoroacetyl)pyrrolidin-3-yl]-1-pyrimidin-5-yl-propyl]-2-methyl-propane-2-sulfinamide

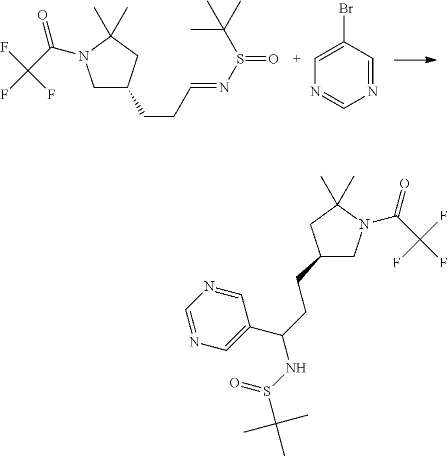

5-Bromopyrimidine (3.2 g, 19.725 mmol) was dissolved in THF (90 mL). The mixture was cooled in an ethanol/liquid nitrogen bath until the bath temperature was <−100° C. and the solution was stirred for 10 min. n-BuLi (8 mL of 2.5 M in hexanes, 20.00 mmol) was added dropwise along the inner wall of the reaction flask. The stirring stopped after several minutes. The mixture was allowed to stay in the cold bath for 20 min. Then N-[3-[(3S)-5,5-dimethyl-1-(2,2,2-trifluoroacetyl)pyrrolidin-3-yl]propylidene]-2-methyl-propane-2-sulfinamide (2.4 g, 6.4328 mmol) in THF (10 mL) was added along the inner wall of the flask over 3 min. The mixture was allowed to warm up in the same bath. The stirring started again when the bath temperature reached ˜−85° C. The reaction was continued at <−70° C. for 15 min. Saturated NH₄Cl (20 mL) was added in one portion. The mixture was allowed to warm up to rt and concentrated. The residue was partitioned between water and EtOAc (30 mL each). The aqueous layer was extracted once with EtOAc. The combined and concentrated EtOAc mixture was purified by a short silica gel column, using 100% EtOAc as eluant, to afford N-[3-[(3S)-5,5-dimethyl-1-(2,2,2-trifluoroacetyl)pyrrolidin-3-yl]-1-pyrimidin-5-yl-propyl]-2-methyl-propane-2-sulfinamide (1 g, 34%). ESI-MS m/z calc. 434.1963, found 435.6 (M+1)⁺; Retention time: 2.61 minutes (LC method B).

### Step 2: 1-[(4S)-4-(3-Amino-3-pyrimidin-5-yl-propyl)-2,2-dimethyl-pyrrolidin-1-yl]-2,2,2-trifluoro-ethanone

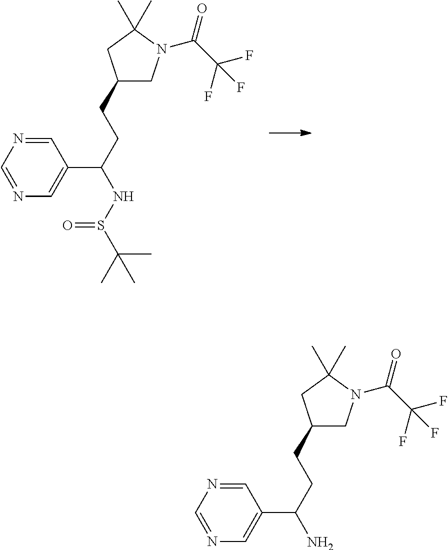 [SMILES: CC1(C)C[C@H](CCC(NS(=O)C(C)(C)C)c2cncnc2)CN1C(=O)C(F)(F)F ; CC1(C)C[C@H](CCC(N)c2cncnc2)CN1C(=O)C(F)(F)F]

N-[3-[(3S)-5,5-Dimethyl-1-(2,2,2-trifluoroacetyl)pyrrolidin-3-yl]-1-pyrimidin-5-yl-propyl]-2-methyl-propane-2-sulfinamide (1.2 g, 2.62 mmol) was dissolved in MeOH (10 mL). An HCl (4 mL of 4 M, 16.00 mmol) solution in dioxane was added. The mixture was stirred at rt for 1 h. It was concentrated and the residue was partitioned between DCM and aqueous NaHCO₃(20 mL each). The aqueous layer was extracted once (f20 mL) with DCM. The combined DCM solution was washed with brine, dried over anhydrous Na₂SO₄, filtered and concentrated to afford 1-[(4S)-4-(3-amino-3-pyrimidin-5-yl-propyl)-2,2-dimethyl-pyrrolidin-1-yl]-2,2,2-trifluoro-ethanone (900 mg, 99%). ESI-MS m/z calc. 330.1667, found 331.4 (M+1)⁺; Retention time: 1.91 minutes (LC method B).

### Step 3: 6-[[3-[(3S)-5,5-Dimethyl-1-(2,2,2-trifluoroacetyl)pyrrolidin-3-yl]-1-pyrimidin-5-yl-propyl]amino]pyridine-2-sulfonamide

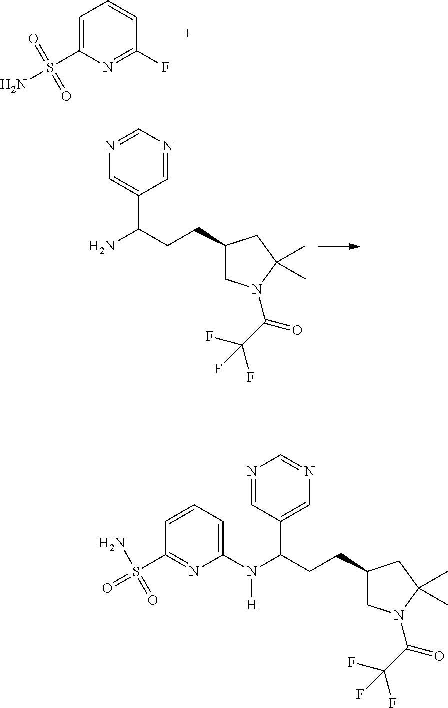 [SMILES: NS(=O)(=O)c1cccc(F)n1 ; CC1(C)C[C@H](CCC(N)c2cncnc2)CN1C(=O)C(F)(F)F ; CC1(C)C[C@H](CCC(Nc2cccc(S(N)(=O)=O)n2)c2cncnc2)CN1C(=O)C(F)(F)F]

1-[(4S)-4-(3-Amino-3-pyrimidin-5-yl-propyl)-2,2-dimethyl-pyrrolidin-1-yl]-2,2,2-trifluoro-ethanone (900 mg, 2.59 mmol) was dissolved in DMSO (2 mL). 6-Fluoropyridine-2-sulfonamide (768 mg, 4.36 mmol) and Na₂CO₃ (868 mg, 8.19 mmol) were added. The mixture was heated in a 110° C. oil bath under a nitrogen balloon for 18 h. It was then cooled to rt and partitioned between EtOAc and water (20 mL each). The organic layer was washed with water (10 mL×2), brine, dried over anhydrous Na₂SO₄, filtered and concentrated. The residue was purified by silica gel chromatography, using 5-80% EtOAc/hexanes to afford 6-[[3-[(3S)-5,5-dimethyl-1-(2,2,2-trifluoroacetyl)pyrrolidin-3-yl]-1-pyrimidin-5-yl-propyl]amino]pyridine-2-sulfonamide (0.504 g, 38%). ¹H NMR (500 MHz, DMSO-d₆) δ 9.03 (s, 1H), 8.89 (s, 2H), 7.65 (d, J=8.2 Hz, 1H), 7.54 (dd, J 8.4,7.2 Hz, 1H), 7.11 (s, 2H), 6.98 (d, J=7.2 Hz, 1H), 6.66 (d, J=8.4 Hz, 1H), 5.18-5.03 (m, 1H), 3.87-3.76 (m, 1H), 3.15 (q, J=11.3 Hz, 1H), 2.34-2.21 (m, 1H), 2.05-1.76 (m, 3H), 1.59-1.48 (m, 1H), 1.47-1.42 (m, 4H), 1.34 (s, 3H), 1.33-1.25 (m, 1H). ESI-MS m/z calc. 486.1661, found 487.3 (M+1)⁺; Retention time: 1.9 minutes (LC method H).

### Step 4: 2-Chloro-N-[[6-[[3-[(3S)-5,5-Dimethyl-1-(2,2,2-trifluoroacetyl)pyrrolidin-3-yl]-1-pyrimidin-5-yl-propyl]amino]-2-pyridyl]sulfonyl]-6-[3-[2-[1-(trifluoromethyl) cyclopropyl]ethoxy]pyrazol-1-yl]pyridine-3-carboxamide

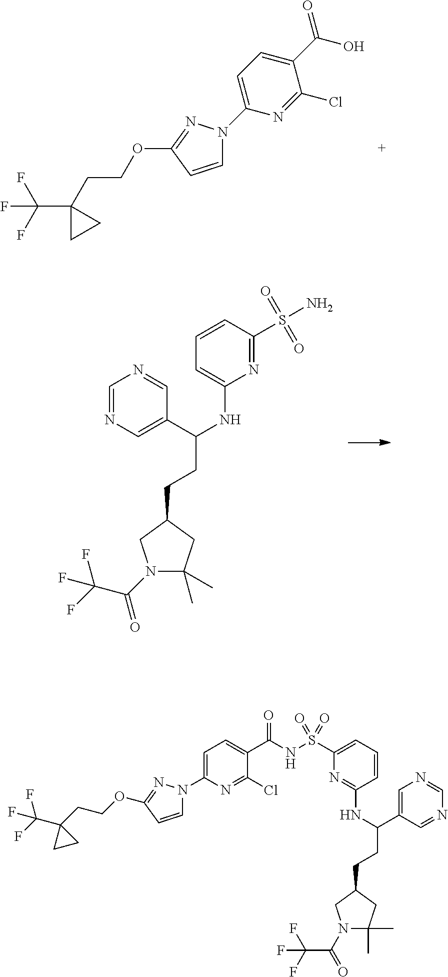 [SMILES: O=C(O)c1ccc(-n2ccc(OCCC3(C(F)(F)F)CC3)n2)nc1Cl ; CC1(C)C[C@H](CCC(Nc2cccc(S(N)(=O)=O)n2)c2cncnc2)CN1C(=O)C(F)(F)F ; CC1(C)C[C@H](CCC(Nc2cccc(S(=O)(=O)NC(=O)c3ccc(-n4ccc(OCCC5(C(F)(F)F)CC5)n4)nc3Cl)n2)c2cncnc2)CN1C(=O)C(F)(F)F]

To a solution of 2-chloro-6-[3-[2-[1-(trifluoromethyl)cyclopropyl]ethoxy] pyrazol-1-yl]pyridine-3-carboxylic acid (447 mg, 1.19 mmol) in THF (12.0 mL) was added CDI (195 mg, 1.203 mmol) (recrystallized from THF) and the mixture was stirred at rt for 2 h then 6-[[3-[(3S)-5,5-dimethyl-1-(2,2,2-trifluoroacetyl)pyrrolidin-3-yl]-1-pyrimidin-5-yl-propyl]amino]pyridine-2-sulfonamide (350 mg, 0.72 mmol) was added followed by DBU (350 μL, 2.34 mmol), and the resulting mixture was stirred for 3 h at rt. The reaction was diluted with ethyl acetate and washed with a saturated aqueous sodium bicarbonate solution, followed by brine. The organics were separated, dried over sodium sulfate, evaporated and then purified on silica gel chromatography (40 gram column) using a gradient from 100% hexanes to 100% ethyl acetate followed by a second silica gel column (40 gram column) using a gradient from 100% dichloromethane to 15% methanol in dichloromethane to afford 2-chloro-N-[[6-[[3-[(3S)-5,5-dimethyl-1-(2,2,2-trifluoroacetyl)pyrrolidin-3-yl]-1-pyrimidin-5-yl-propyl]amino]-2-pyridyl]sulfonyl]-6-[3-[2-[1-(trifluoromethyl)cyclopropyl]ethoxy]pyrazol-1-yl]pyridine-3-carboxamide (568 mg, 94%) as an off-white solid. ESI-MS m/z calc. 843.2153, found 844.2 (M+1)⁺; Retention time: 2.07 minutes (LC method A).

### Step 5: 2-Chloro-N-[[6-[[3-[(3S)-5,5-Dimethylpyrrolidin-3-yl]-1-pyrimidin-5-yl-propyl]amino]-2-pyridyl]sulfonyl]-6-[3-[2-[1-(trifluoromethyl)cyclopropyl] ethoxy]pyrazol-1-yl]pyridine-3-carboxamide

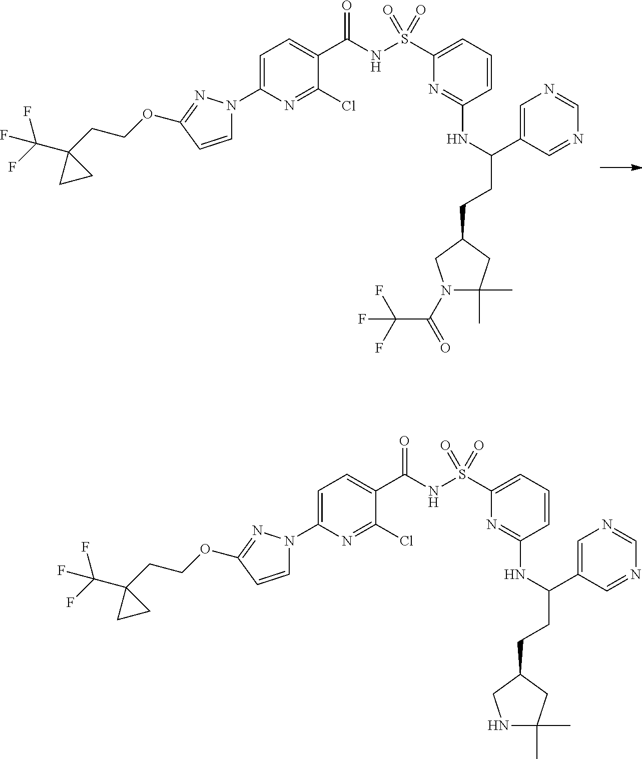 [SMILES: CC1(C)C[C@H](CCC(Nc2cccc(S(=O)(=O)NC(=O)c3ccc(-n4ccc(OCCC5(C(F)(F)F)CC5)n4)nc3Cl)n2)c2cncnc2)CN1C(=O)C(F)(F)F ; CC1(C)C[C@H](CCC(Nc2cccc(S(=O)(=O)NC(=O)c3ccc(-n4ccc(OCCC5(C(F)(F)F)CC5)n4)nc3Cl)n2)c2cncnc2)CN1]

A mixture of 2-chloro-N-[[6-[[3-[(3S)-5,5-dimethyl-1-(2,2,2-trifluoroacetyl)pyrrolidin-3-yl]-1-pyrimidin-5-yl-propyl]amino]-2-pyridyl]sulfonyl]-6-[3-[2-[1-(trifluoromethyl)cyclopropyl]ethoxy]pyrazol-1-yl]pyridine-3-carboxamide (568 mg, 0.67 mmol) and K₂CO₃ (950 mg, 6.87 mmol) in methanol (25 mL) and water (10 mL) was stirred at rt for 12 hours. The mixture was heated at 70° C. for 2 hours. A second portion of K₂CO₃ (950 mg, 6.87 mmol) was added and the reaction was heated at 90° C. for 4 hours. The mixture was concentrated in vacuo and the residue was partitioned between water and EtOAc. The organic layer was separated and the aqueous layer was re-extracted with ethyl acetate. The combined organic layers were washed with brine, separated, and dried over sodium sulfate. The mixture was evaporated and then suspended in diethyl ether (10 mL) and HCl (175 μL of 4 M in dioxane, 0.70 mmol) was added. The material was concentrated to dryness to afford an off-white solid 2-chloro-N-[[6-[[3-[(3S)-5,5-dimethylpyrrolidin-3-yl]-1-pyrimidin-5-yl-propyl]amino]-2-pyridyl]sulfonyl]-6-[3-[2-[1-(trifluoromethyl)cyclopropyl]ethoxy]pyrazol-1-yl]pyridine-3-carboxamide (dihydrochloride salt) (500 mg, 91%). ¹H NMR (499 MHz, DMSO-d₆) δ 12.81 (s, 1H), 9.12 (s, 1H), 9.05 (s, 1H), 9.01 (d, J=0.8 Hz, 1H), 8.85 (s, 2H), 8.42 (dd, J=2.8, 1.7 Hz, 1H), 8.05 (d, J=8.3 Hz, 2H), 7.71 (dd, J=8.4, 1.2 Hz, 1H), 7.66 (ddd, J=8.1, 7.0, 0.9 Hz, 1H), 7.21 (dd, J=7.1, 1.9 Hz, 1H), 6.87 (d, J=8.5 Hz, 1H), 6.20 (d, J=2.8 Hz, 1H), 4.92 (s, 1H), 4.36 (t, J=7.1 Hz, 2H), 3.34 (q, J=11.1, 8.7 Hz, 1H), 2.84 (ddt, J=27.3, 8.7, 5.0 Hz, 1H), 2.54 (s, 1H), 2.45-2.31 (m, 1H), 2.14-2.05 (m, 2H), 1.94 (dt, J=13.3, 7.1 Hz, 2H), 1.81 (ddd, J=10.8, 8.2, 5.4 Hz, 1H), 1.62-1.46 (m, 1H), 1.43-1.37 (m, 1H), 1.36 (d, J=3.3 Hz, 3H), 1.35-1.28 (m, 1H), 1.26 (d, J=2.5 Hz, 3H), 0.99-0.93 (m, 2H), 0.90 (dt, J=5.0, 1.7 Hz, 2H). ESI-MS m/z calc. 747.233, found 748.2 (M+1)⁺; Retention time: 1.57 minutes (LC method A).

### Step 6: (14S)-12,12-Dimethyl-17-(pyrimidin-5-yl)-8-(3-{2-[1-(trifluoromethyl) cyclopropyl]ethoxy}-1H-pyrazol-1-yl)-2λ⁶-thia-3,9,11,18,23-pentaazatetracyclo [17.3.1.111,14.05,10]tetracosa-1(22),5,7,9,19(23),20-hexaene-2,2,4-trione

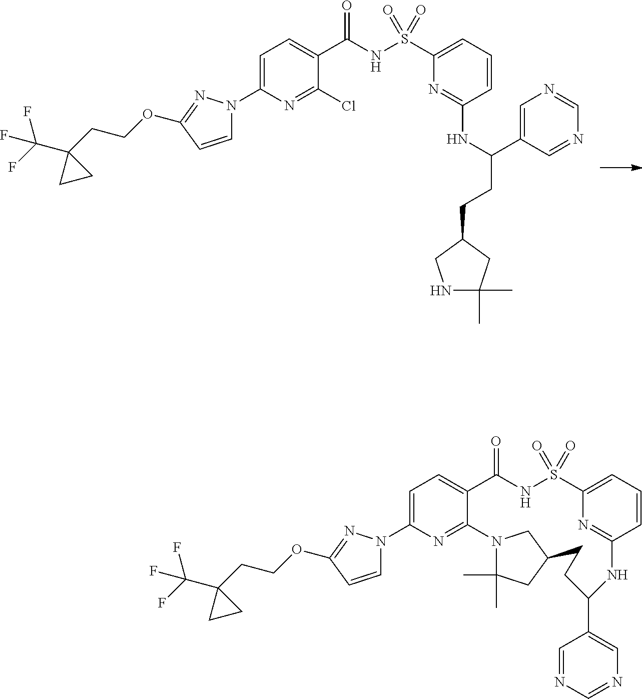 [SMILES: CC1(C)C[C@H](CCC(Nc2cccc(S(=O)(=O)NC(=O)c3ccc(-n4ccc(OCCC5(C(F)(F)F)CC5)n4)nc3Cl)n2)c2cncnc2)CN1 ; CC1(C)C[C@@H]2CCC(c3cncnc3)Nc3cccc(n3)S(=O)(=O)NC(=O)c3ccc(-n4ccc(OCCC5(C(F)(F)F)CC5)n4)nc3N1C2]

A mixture of 2-chloro-N-[[6-[[3-[(3S)-5,5-dimethylpyrrolidin-3-yl]-1-pyrimidin-5-yl-propyl]amino]-2-pyridyl]sulfonyl]-6-[3-[2-[1-(trifluoromethyl)cyclopropyl]ethoxy]pyrazol-1-yl]pyridine-3-carboxamide (hydrochloride salt) (500 mg, 0.64 mmol), K₂CO₃ (650 mg, 4.70 mmol), 3 Å molecular sieves and DMSO (13 mL) in a 20 mL microwave vial was purged with nitrogen, capped, heated to 155° C. and stirred for 18 h. The mixture was cooled to rt, filtered and then acidified with saturated aqueous citric acid. Then ethyl acetate was added, and the aqueous layer was extracted (3×50 mL). The combined organic extract was washed with water (50 mL) and saturated brine solution (50 mL), then dried over sodium sulfate, filtered, and evaporated in vacuo. This crude product was purified by silica gel chromatography using a 80 g column eluting with 100% dichloromethane to 15% methanol in dichloromethane to afford an off-white solid, (14S)-12,12-dimethyl-17-(pyrimidin-5-yl)-8-(3-{2-[1-(trifluoromethyl)cyclopropyl] ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (135 mg, 28%). ESI-MS m/z calc. 711.2563, found 712.2 (M+1)⁺; Retention time: 2.04 minutes (LC method A).

### Step 7: (14S)-12,12-Dimethyl-17-(pyrimidin-5-yl)-8-(3-{2-[1-(trifluoromethyl) cyclopropyl]ethoxy}-1H-pyrazol-1-yl)-2λ⁶-thia-3,9,11,18,23-pentaazatetracyclo [17.3.1.111,14.05,10]tetracosa-1(22),5,7,9,19(23),20-hexaene-2,2,4-trione (diastereomer 1, Compound 375) and (14S)-12,12-dimethyl-17-(pyrimidin-5-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (diastereomer 2, Compound 374)

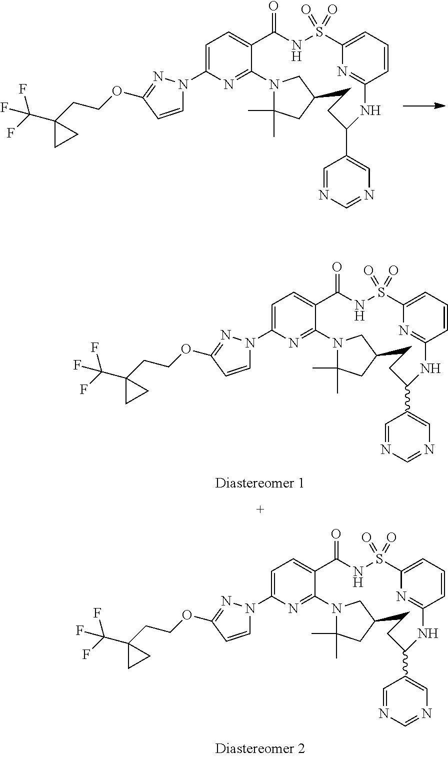 [SMILES: CC1(C)C[C@@H]2CCC(c3cncnc3)Nc3cccc(n3)S(=O)(=O)NC(=O)c3ccc(-n4ccc(OCCC5(C(F)(F)F)CC5)n4)nc3N1C2 ; CC1(C)C[C@@H]2CCC(c3cncnc3)Nc3cccc(n3)S(=O)(=O)NC(=O)c3ccc(-n4ccc(OCCC5(C(F)(F)F)CC5)n4)nc3N1C2 ; CC1(C)C[C@@H]2CCC(c3cncnc3)Nc3cccc(n3)S(=O)(=O)NC(=O)c3ccc(-n4ccc(OCCC5(C(F)(F)F)CC5)n4)nc3N1C2]

(14S)-12,12-Dimethyl-17-(pyrimidin-5-yl)-8-(3-[2-[1-(trifluoromethyl) cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (mixture of two diastereomers, 130 mg, 0.1826 mmol) was subjected to SFC using the following method: ChiralPaK IC (250×21.2 mm, 5 μm) column, 40° C., mobile phase 41% MeOH, 59% CO₂, flow 70 mL/min, concentrations 32.5 mg/mL in MeOH, injection volume 500 μL, pressure 166 bar, wavelength 278 nm. Two diastereomers were separated and isolated as off-white solids:

Diastereomer 1: (14S)-12,12-dimethyl-17-(pyrimidin-5-yl)-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (48.8 mg, 74%). ¹H NMR (500 MHz, DMSO-d₆) δ 12.57 (s, 1H), 9.06 (s, 1H), 8.88 (s, 2H), 8.23 (d, J=2.7 Hz, 1H), 7.91 (d, J=8.3 Hz, 1H), 7.79 (d, J=8.7 Hz, 1H), 7.66 (t, J=7.9 Hz, 1H), 7.13 (d, J=7.2 Hz, 1H), 6.95 (d, J=8.2 Hz, 1H), 6.80 (d, J=8.5 Hz, 1H), 6.12 (d, J=2.7 Hz, 1H), 5.23 (t, J=11.1 Hz, 1H), 4.32 (t, J=7.0 Hz, 2H), 2.79 (t, J=10.4 Hz, 1H), 2.30 (d, J=17.4 Hz, 1H), 2.14-2.02 (m, 3H), 1.91-1.85 (m, 1H), 1.78 (t, J=13.4 Hz, 2H), 1.63 (s, 3H), 1.60 (d, J=12.4 Hz, 1H), 1.53 (s, 3H), 1.51-1.45 (m, 1H), 1.23 (s, 1H), 0.99-0.92 (m, 2H), 0.91-0.84 (m, 2H). ESI-MS m/z calc. 711.2563, found 712.2 (M+1)⁺; Retention time: 2.04 minutes (LC method A).

Diastereomer 2: (14S)-12,12-dimethyl-17-(pyrimidin-5-yl)-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (60.81 mg, 93%). ¹H NMR (500 MHz, DMSO-d₆) δ 12.47 (s, 1H), 9.07 (s, 1H), 8.98 (s, 2H), 8.21 (d, J=2.7 Hz, 1H), 7.84 (s, 1H), 7.73 (s, 1H), 7.65 (t, J=7.9 Hz, 1H), 7.25 (d, J=7.3 Hz, 1H), 6.92 (d, J=8.2 Hz, 1H), 6.78 (d, J=8.5 Hz, 1H), 6.10 (d, J 2.7 Hz, 1H), 4.84 (s, 1H), 4.31 (t, J=7.1 Hz, 2H), 2.99 (s, 1H), 2.38-2.27 (m, 1H), 2.08 (t, J=7.1 Hz, 2H), 1.98 (d, J=16.9 Hz, 1H), 1.89 (dd, J=11.9, 5.7 Hz, 1H), 1.80-1.70 (m, 1H), 1.59 (s, 3H), 1.56 (s, 1H), 1.54 (s, 3H), 1.24 (d, J=4.8 Hz, 2H), 1.13 (d, J=14.0 Hz, 1H), 0.97-0.91 (m, 2H), 0.91-0.84 (m, 2H). ESI-MS m/z calc. 711.2563, found 712.2 (M+1)⁺; Retention time: 2.04 minutes (LC method A).

### Example 127: Preparation of 12,12-dimethyl-15-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 394 (enantiomer 1), Compound 393 (enantiomer 2), Compound 386 (enantiomer 3), and Compound 385 (enantiomer 4)

[chemical structure]

### Step 1: tert-Butyl N-(3-oxo-3-phenyl-propyl)carbamate

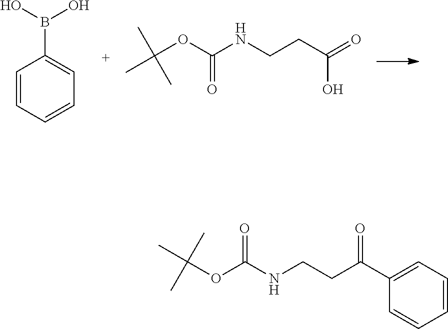 [SMILES: OB(O)c1ccccc1 ; CC(C)(C)OC(=O)NCCC(=O)O ; CC(C)(C)OC(=O)NCCC(=O)c1ccccc1]

Palladium acetate (1.1213 g, 4.99 mmol) and triphenylphosphine (2.620 g, 9.99 mmol) were dissolved in THF (500 mL). To the resulting solution were added 3-(tert-butoxycarbonylamino)propanoic acid (18.9 g, 99.89 mmol), phenylboronic acid (24.359 g, 199.78 mmol), water (5 mL) and trimethylacetic anhydride (27.906 g, 149.83 mmol), and the mixture was stirred at 60° C. for 24 hours. The mixture was filtered, saturated sodium bicarbonate (500 mL) was added and the mixture was extracted with ethyl acetate (3×300 mL). The organic fractions were combined, dried over sodium sulfate end evaporated, the residue was purified by silica gel column chromatography using 0-10% ethyl acetate in hexanes to give tert-butyl N-(3-oxo-3-phenyl-propyl)carbamate (18.42 g, 72%) as an off-yellow solid. ESI-MS m/z calc. 249.1365, found 250.2 (M+1)⁺; Retention time: 2.8 minutes (LC method B).

### Step 2: tert-Butyl N-[(3Z)-3-[1-[(4-methoxyphenyl)methyl]-5,5-dimethyl-2-oxo-pyrrolidin-3-ylidene]-3-phenyl-propyl]carbamate, Isomer A and tert-butyl N-[(3E)-3-[1-[(4-methoxyphenyl)methyl]-5,5-dimethyl-2-oxo-pyrrolidin-3-ylidene]-3-phenyl-propyl]carbamate, Isomer B

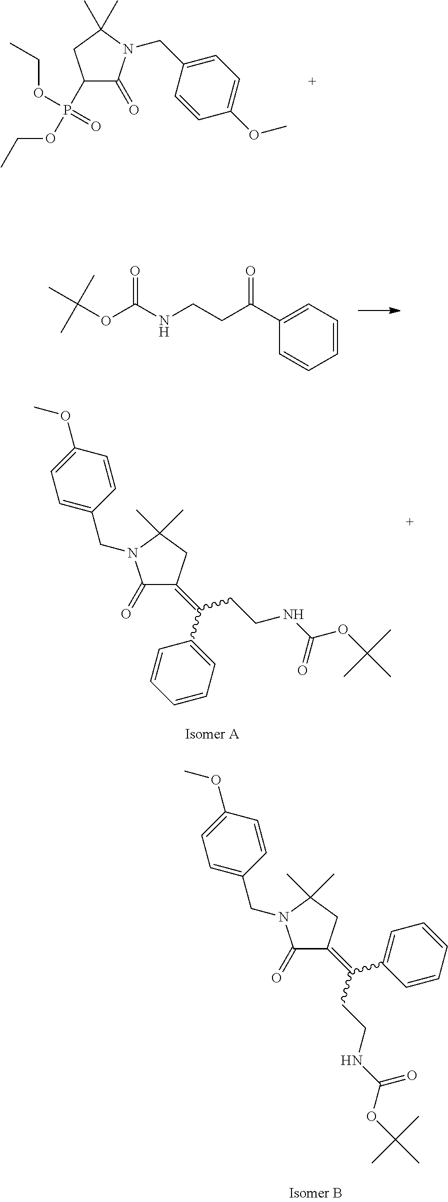

3-Diethoxyphosphoryl-1-[(4-methoxyphenyl)methyl]-5,5-dimethyl-pyrrolidin-2-one (5 g, 13.54 mmol) was dissolved in tetrahydrofuran (40 mL) and cooled in an ice water bath. NaH (60%, 730.89 mg, 18.274 mmol) was added and the resulting mixture was stirred for 30 minutes. tert-Butyl N-(3-oxo-3-phenyl-propyl)carbamate (4.2183 g, 16.92 mmol) was dissolved in tetrahydrofuran (10 mL) and the resulting solution was added dropwise to the cooled phosphonate solution. The ice water bath was removed and the resulting mixture was stirred at room temperature for 16 hours, then was diluted with water (50 mL) and extracted with ethyl acetate (3×30 mL). The organic fractions were combined, dried over sodium sulfate and evaporated; the residue was purified by silica gel column chromatography using 0-20% ethyl acetate in hexanes. Two isomers with an unknown Z or E configuration were isolated. A configuration was arbitrarily attributed to structures of each isomer.

Isomer A (Less Polar):

tert-Butyl N-[(3Z)-3-[1-[(4-methoxyphenyl)methyl]-5,5-dimethyl-2-oxo-pyrrolidin-3-ylidene]-3-phenyl-propyl]carbamate (1.725 g, 26%). ¹H NMR (250 MHz, CDCl₃) δ 7.51-7.02 (m, 8H), 6.96-6.60 (m, 2H), 4.44 (s, 2H), 3.79 (s, 3H), 3.39-2.97 (m, 4H), 2.41 (s, 2H), 1.42 (s, 9H), 1.08 (s, 6H). ESI-MS m/z calc. 464.2675, found 465.1 (M+1)⁺; Retention time: 3.64 minutes (LC method B).

Isomer B (More Polar):

tert-Butyl N-[(3E)-3-[1-[(4-methoxyphenyl)methyl]-5,5-dimethyl-2-oxo-pyrrolidin-3-ylidene]-3-phenyl-propyl]carbamate (3.981 g, 62%). ¹H NMR (250 MHz, CDCl₃) δ 7.60-6.95 (m, 7H), 6.95-6.54 (m, 2H), 4.54 (s, 1H), 4.36 (s, 2H), 3.76 (s, 3H), 3.23-2.92 (m, 2H), 2.76-2.46 (m, 4H), 1.42 (s, 9H), 1.18 (s, 6H). ESI-MS m/z calc. 464.2675, found 465.2 (M+1)⁺; Retention time: 3.34 minutes (LC method B).

### Step 3: tert-Butyl N-[3-[1-[(4-methoxyphenyl)methyl]-5,5-dimethyl-2-oxo-pyrrolidin-3-yl]-3-phenyl-propyl]carbamate

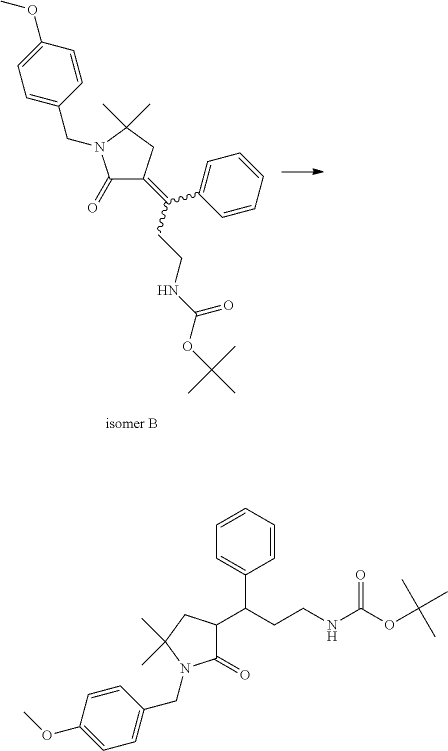

A solution of tert-butyl N-[(3E)-3-[1-[(4-methoxyphenyl)methyl]-5,5-dimethyl-2-oxo-pyrrolidin-3-ylidene]-3-phenyl-propyl]carbamate, isomer B (6.881 g, 14.81 mmol, isomer B) in MeOH (100 mL) was placed in a Parr reactor, palladium on carbon (1.5762 g, 10% w/w, 1.4811 mmol) was added. The reactor was flushed with nitrogen, then with hydrogen and was shaken for 48 hours under hydrogen (50 psi). The mixture was filtered through Celite, evaporated, the residue was purified by silica gel column chromatography using 0-20% ethyl acetate in hexanes to give tert-butyl N-[(3S)-3-[(3S)-1-[(4-methoxyphenyl)methyl]-5,5-di methyl-2-oxo-pyrrolidin-3-yl]-3-phenyl-propyl]carbamate (5.815 g, 72%). ESI-MS m/z calc. 466.2832, found 467.2 (M+1)⁺; Retention time: 3.29 minutes (LC method B).

### Step 4: 3-(3-Amino-1-phenyl-propyl)-5,5-dimethyl-pyrrolidin-2-one

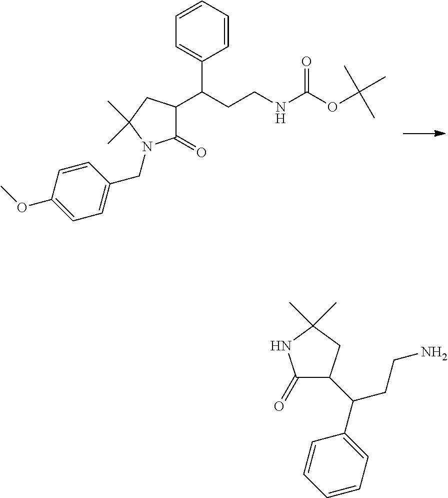 [SMILES: COc1ccc(CN2C(=O)C(C(CCNC(=O)OC(C)(C)C)c3ccccc3)CC2(C)C)cc1 ; CC1(C)CC(C(CCN)c2ccccc2)C(=O)N1]

tert-Butyl N-[3-[1-[(4-methoxyphenyl)methyl]-5,5-dimethyl-2-oxo-pyrrolidin-3-yl]-3-phenyl-propyl]carbamate (5.815 g, 12.46 mmol) was dissolved in TFA (20 mL, 259.60 mmol) and the mixture was heated in a microwave reactor at 100° C. for 26 hours. The resulting mixture was evaporated to give crude 3-(3-amino-1-phenyl-propyl)-5,5-dimethyl-pyrrolidin-2-one (trifluoroacetate salt) (4.5 g, 75%). ESI-MS m/z calc. 246.17322, found 247.1 (M+1)⁺; Retention time: 1.7 minutes (LC method B).

### Step 5: 3-[3-(Dibenzylamino)-1-phenyl-propyl]-5,5-dimethyl-pyrrolidin-2-one

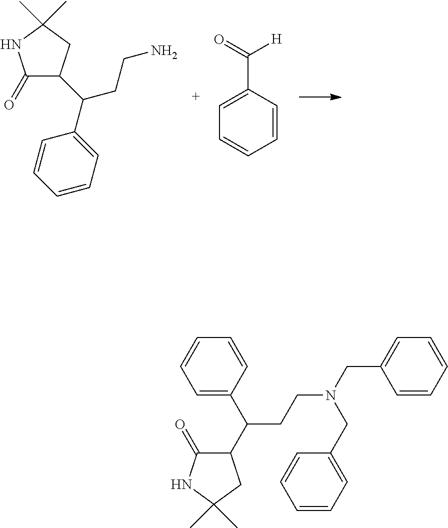 [SMILES: CC1(C)CC(C(CCN)c2ccccc2)C(=O)N1 ; O=Cc1ccccc1 ; CC1(C)CC(C(CCN(Cc2ccccc2)Cc2ccccc2)c2ccccc2)C(=O)N1]

To a solution of 3-(3-amino-1-phenyl-propyl)-5,5-dimethyl-pyrrolidin-2-one (trifluoroacetate salt) (4.5 g, 9.36 mmol) in 1,2-dichloroethane (200 mL), benzaldehyde (2.4846 g, 23.413 mmol) was added followed by sodium triacetoxyborohydride (11.909 g, 56.192 mmol). The mixture was stirred at room temperature for 2 hours and quenched with a saturated aqueous K₂CO₃ solution (100 mL). The organic phase was separated, and the aqueous phase was extracted with chloroform (100 mL). The organic fractions were combined, dried over sodium sulfate and evaporated, then the residue was purified by silica gel column chromatography using 0-50% ethyl acetate in hexanes to give 3-[3-(dibenzylamino)-1-phenyl-propyl]-5,5-dimethyl-pyrrolidin-2-one (3.8957 g, 93%). ESI-MS m/z calc. 426.26712, found 427.1 (M+1)⁺; Retention time: 2.52 minutes (LC method B).

### Step 6: tert-Butyl 4-[3-(dibenzylamino)-1-phenyl-propyl]-2,2-dimethyl-pyrrolidine-1-carboxylate

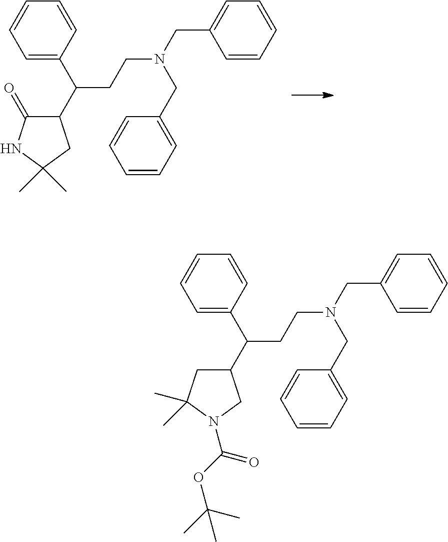 [SMILES: CC1(C)CC(C(CCN(Cc2ccccc2)Cc2ccccc2)c2ccccc2)C(=O)N1 ; CC(C)(C)OC(=O)N1CC(C(CCN(Cc2ccccc2)Cc2ccccc2)c2ccccc2)CC1(C)C]

To 3-[3-(dibenzylamino)-1-phenyl-propyl]-5,5-dimethyl-pyrrolidin-2-one (3.8957 g, 9.1321 mmol), borane dimethyl sulfide complex (50 mL of 2 M in THF, 100.00 mmol) was added under argon and the mixture was stirred at 70° C. for 16 hours. The mixture was cooled to room temperature and methanol (100 mL) was slowly added, followed by 6M HCl (50 mL). The mixture was stirred at 70° C. for 1 hour and evaporated. To the residue chloroform (100 mL) was added followed by aqueous NaOH (100 mL, 15% w/w) and BOC anhydride (2.1923 g, 10.045 mmol). The mixture was stirred at room temperature for 1 hour, then the organic phase was separated and the aqueous phase was extracted with chloroform (2×50 mL). Organic fractions were combined, dried over sodium sulfate and evaporated, then the residue was purified by silica gel column chromatography using 0-10% ethylacetate in hexanes to give tert-butyl 4-[3-(dibenzylamino)-1-phenyl-propyl]-2,2-dimethyl-pyrrolidine-1-carboxylate (3.91 g, 79%) as a colorless oil. ESI-MS m/z calc. 512.3403, found 513.4 (M+1)⁺; Retention time: 3.19 minutes (LC method B).

### Step 7: tert-Butyl 4-(3-amino-1-phenyl-propyl)-2,2-dimethyl-pyrrolidine-1-carboxylate

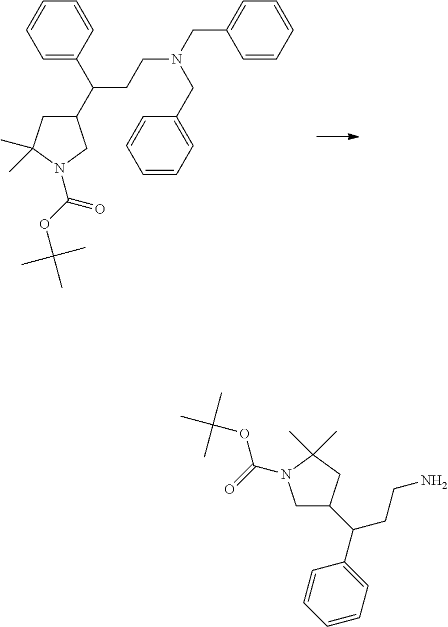 [SMILES: CC(C)(C)OC(=O)N1CC(C(CCN(Cc2ccccc2)Cc2ccccc2)c2ccccc2)CC1(C)C ; CC(C)(C)OC(=O)N1CC(C(CCN)c2ccccc2)CC1(C)C]

tert-Butyl 4-[3-(dibenzylamino)-1-phenyl-propyl]-2,2-dimethyl-pyrrolidine-1-carboxylate (3.91 g, 7.6259 mmol) was dissolved in methanol (100 mL). Ammonium formate (1.9235 g, 30.504 mmol) was added followed by palladium on carbon (0.4 g, 10% w/w, 0.3759 mmol). The mixture was stirred under argon at 65° C. for 3 hours. After cooling, it was filtered through Celite and concentrated, then the residue was purified by silica gel column chromatography using 0-5% methanol in dichloromethane to give tert-butyl 4-(3-amino-1-phenyl-propyl)-2,2-dimethyl-pyrrolidine-1-carboxylate (2.446 g, 86%). ESI-MS m/z calc. 332.24637, found 333.0 (M+1)⁺; Retention time: 2.57 minutes (LC method B).

### Step 8: tert-Butyl 2,2-dimethyl-4-[1-phenyl-3-[(6-sulfamoyl-2-pyridyl)amino]propyl]pyrrolidine-1-carboxylate

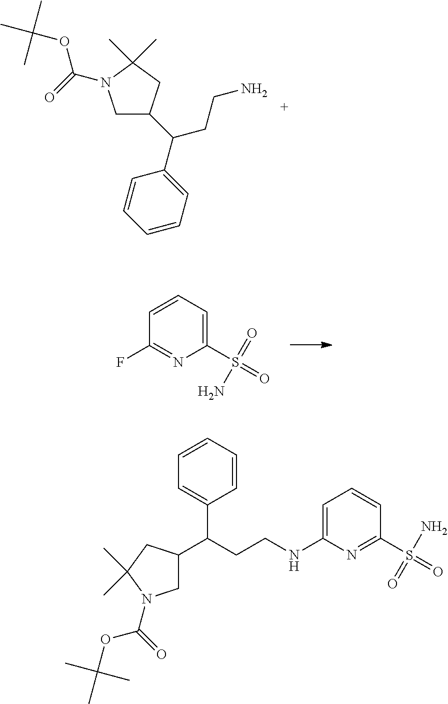 [SMILES: CC(C)(C)OC(=O)N1CC(C(CCN)c2ccccc2)CC1(C)C ; NS(=O)(=O)c1cccc(F)n1 ; CC(C)(C)OC(=O)N1CC(C(CCNc2cccc(S(N)(=O)=O)n2)c2ccccc2)CC1(C)C]

tert-Butyl 4-(3-amino-1-phenyl-propyl)-2,2-dimethyl-pyrrolidine-1-carboxylate (2.38 g, 7.16 mmol) and 6-fluoropyridine-2-sulfonamide (1.8915 g, 10.74 mmol) were dissolved in DMSO (71.4 mL), then sodium carbonate (2.2761 g, 21.475 mmol) was added and the mixture was stirred under argon at 100° C. for 7 hours. The mixture was quenched with water (350 mL) and extracted with EtOAc (3×100 mL). The organic fractions were combined, dried over sodium sulfate and evaporated, then the residue was purified by silica gel column chromatography using 0-50% ethyl acetate in hexanes to give tert-butyl 2,2-dimethyl-4-[1-phenyl-3-[(6-sulfamoyl-2-pyridyl)amino]propyl]pyrrolidine-1-carboxylate (3.1304 g, 84%). ¹H NMR (250 MHz, DMSO-d₆) δ 7.51 (t, J=7.8 Hz, 1H), 7.44-7.14 (m, 5H), 7.03 (s, 2H), 6.96 (d, J=7.2 Hz, 1H), 6.93-6.84 (m, 1H), 6.54 (d, J=8.5 Hz, 1H), 3.83-3.57 (m, 1H), 3.14-2.84 (m, 3H), 2.48-2.28 (m, 2H), 1.95-1.77 (m, 2H), 1.39 (s, 9H), 1.32-1.21 (m, 5H), 1.16 (s, 3H). ESI-MS m/z calc. 488.24573, found 489.4 (M+1)⁺; Retention time: 2.77 minutes (LC method C).

### Step 9: tert-Butyl 4-[3-[[6-[[2-chloro-6-[3-[2-[1-(trifluoromethyl)cyclopropyl]ethoxy] pyrazol-1-yl]pyridine-3-carbonyl]sulfamoyl]-2-pyridyl]amino]-1-phenyl-propyl]-2,2-dimethyl-pyrrolidine-1-carboxylate

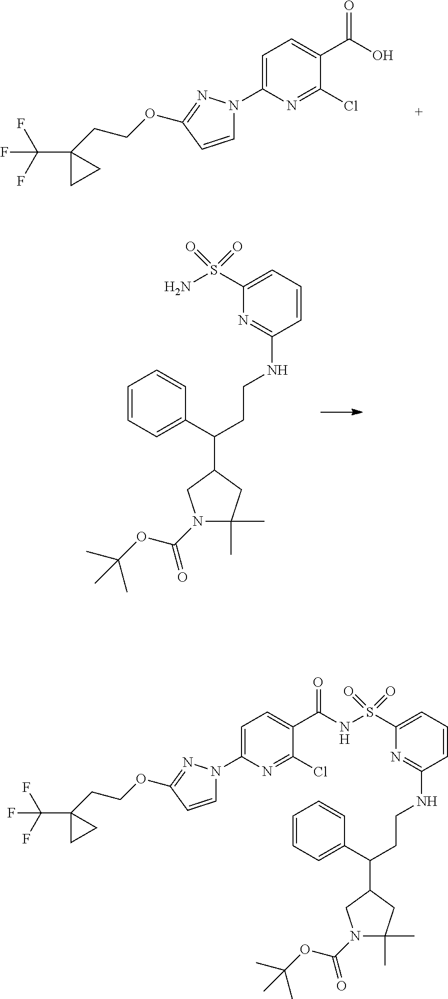 [SMILES: O=C(O)c1ccc(-n2ccc(OCCC3(C(F)(F)F)CC3)n2)nc1Cl ; CC(C)(C)OC(=O)N1CC(C(CCNc2cccc(S(N)(=O)=O)n2)c2ccccc2)CC1(C)C ; CC(C)(C)OC(=O)N1CC(C(CCNc2cccc(S(=O)(=O)NC(=O)c3ccc(-n4ccc(OCCC5(C(F)(F)F)CC5)n4)nc3Cl)n2)c2ccccc2)CC1(C)C]

To a solution of 2-chloro-6-[3-[2-[1-(trifluoromethyl)cyclopropyl]ethoxy] pyrazol-1-yl]pyridine-3-carboxylic acid (592 mg, 1.576 mmol) in THF (17 mL) was added CDI (255 mg, 1.573 mmol) (recrystallized from THF) and the mixture was stirred at rt for 1.5h. Then tert-butyl 2,2-dimethyl-4-[1-phenyl-3-[(6-sulfamoyl-2-pyridyl)amino] propyl]pyrrolidine-1-carboxylate (512 mg, 1.048 mmol) was added as a solution in THF (9 mL) followed by DBU (515 mg, 3.383 mmol), and the resulting mixture was stirred for 3h at rt. The reaction was diluted with ethyl acetate and washed with saturated aqueous sodium bicarbonate solution, followed by brine. The organics were separated, dried over sodium sulfate, evaporated and then purified on silica gel chromatography (40 gram column) using a gradient from 100% dichloromethane to 15% methanol in dichloromethane followed by a second silica gel column (40 gram column) using a gradient from 100% hexanes to 80% ethyl acetate in hexanes to afford tert-butyl 4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]-1-phenyl-propyl]-2,2-dimethyl-pyrrolidine-1-carboxylate as a white solid (650 mg, 73%). ¹H NMR (400 MHz, DMSO-d₆) δ 12.71 (s, 1H), 8.42 (t, J=2.9 Hz, 1H), 8.01 (dd, J=8.3, 2.9 Hz, 1H), 7.69 (d, J=8.3 Hz, 1H), 7.61 (dd, J=8.5, 7.2 Hz, 1H), 7.32-7.24 (m, 2H), 7.24-7.15 (m, 4H), 7.12 (t, J=5.0 Hz, 1H), 6.70 (d, J=8.5 Hz, 1H), 6.20 (t, J=2.5 Hz, 1H), 4.37 (t, J=7.1 Hz, 2H), 3.68 (ddd, J=22.3, 10.7, 7.3 Hz, 1H), 2.95 (qd, J=15.5, 12.9, 4.8 Hz, 3H), 2.47 (d, J=7.9 Hz, 1H), 2.44-2.30 (m, 1H), 2.10 (t, J=7.1 Hz, 2H), 1.88 (dd, J=12.7, 5.9 Hz, 2H), 1.37 (d, J=3.6 Hz, 9H), 1.35-1.29 (m, 1H), 1.25 (d, J=14.4 Hz, 4H), 1.13 (s, 3H), 0.99-0.92 (m, 2H), 0.90 (d, J=9.5 Hz, 2H). ESI-MS m/z calc. 845.2949, found 846.2 (M+1)⁺; Retention time: 2.12 minutes (LC method A).

### Step 10: 12,12-Dimethyl-15-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione

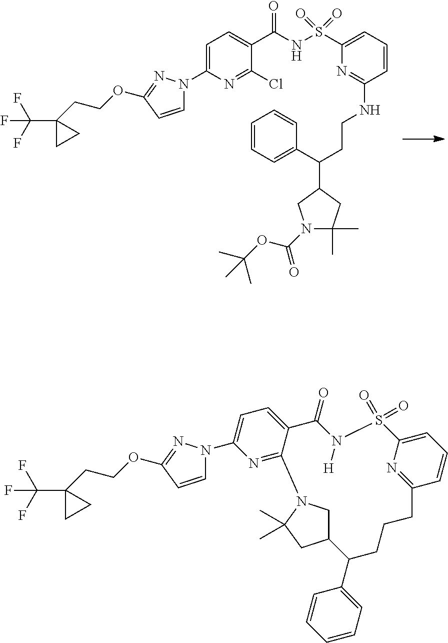 [SMILES: CC(C)(C)OC(=O)N1CC(C(CCNc2cccc(S(=O)(=O)NC(=O)c3ccc(-n4ccc(OCCC5(C(F)(F)F)CC5)n4)nc3Cl)n2)c2ccccc2)CC1(C)C ; CC1(C)CC2CN1c1nc(-n3ccc(OCCC4(C(F)(F)F)CC4)n3)ccc1C(=O)NS(=O)(=O)c1cccc(n1)CCCC2c1ccccc1]

Stage 1:

tert-Butyl 4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]-1-phenyl-propyl]-2,2-dimethyl-pyrrolidine-1-carboxylate (650 mg, 0.7680 mmol) was dissolved in DCM (20 mL) and to the mixture was added HCl (4 M in dioxane) (5.0 mL of 4 M, 20.00 mmol) and stirred at room temperature. After 16 h, the mixture was evaporated to dryness, then diluted with diethyl ether (5 mL×2), and concentrated. The material was purified on silica gel chromatography (40 gram column) using a gradient from 100% dichloromethane to 25% methanol in dichloromethane, concentrated and then placed on the high vacuum pump for 2 h to afford the intermediate 2-chloro-N-[[6-[[3-(5,5-dimethylpyrrolidin-3-yl)-3-phenyl-propyl]amino]-2-pyridyl]sulfonyl]-6-[3-[2-[1-(trifluoromethyl)cyclopropyl] ethoxy]pyrazol-1-yl]pyridine-3-carboxamide (hydrochloride salt) as an off-white solid (329 mg, 55%). ESI-MS m/z calc. 745.2425, found 746.2 (M+1)⁺; Retention time: 1.67 minutes (LC method G).

Stage 2:

Combined material from Stage 1 and K₂CO₃ (770 mg, 5.571 mmol), 3 Å molecular sieves and DMSO (20 mL) in a vial, purged with nitrogen, capped, heated to 155° C. and stirred for 16 h. The mixture was filtered and concentrated under a stream of nitrogen to give a residue which was purified by reverse-phase preparative chromatography utilizing a C₁₈ column and a 30-99% gradient over 15 min of acetonitrile in water (+5 mM HCl) to afford as an off-white solid 12,12-dimethyl-15-phenyl-8-(3-{2-[1-(trifluoromethyl)cyclo propyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (270 mg, 50%). ESI-MS m/z calc. 709.2658, found 710.2 (M+1)⁺; Retention time: 1.84 minutes (LC method G).

### Step 11: 12,12-Dimethyl-15-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 394 (enantiomer 1) and 12,12-dimethyl-15-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione Compound 393 (enantiomer 2)

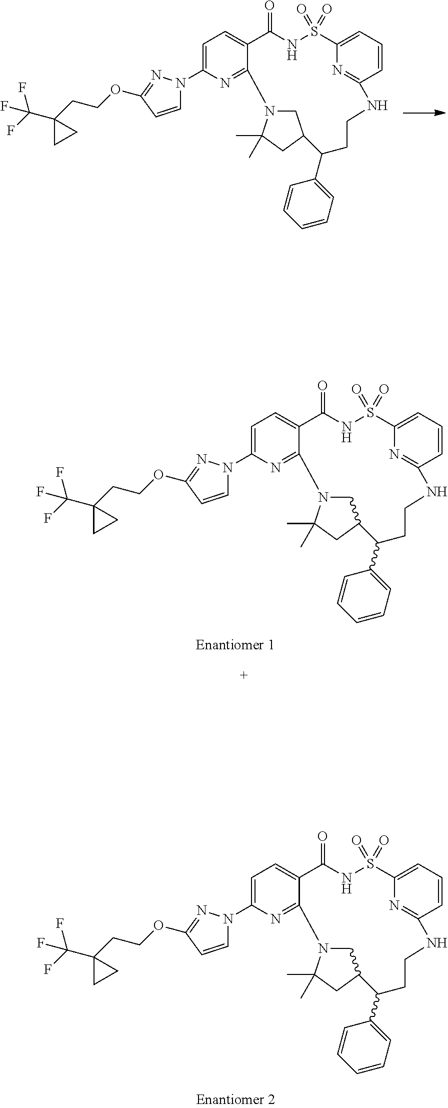 [SMILES: CC1(C)CC2CN1c1nc(-n3ccc(OCCC4(C(F)(F)F)CC4)n3)ccc1C(=O)NS(=O)(=O)c1cccc(n1)NCCC2c1ccccc1 ; CC1(C)CC2CN1c1nc(-n3ccc(OCCC4(C(F)(F)F)CC4)n3)ccc1C(=O)NS(=O)(=O)c1cccc(n1)NCCC2c1ccccc1 ; CC1(C)CC2CN1c1nc(-n3ccc(OCCC4(C(F)(F)F)CC4)n3)ccc1C(=O)NS(=O)(=O)c1cccc(n1)NCCC2c1ccccc1]

12,12-Dimethyl-15-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (mixture of two enantiomers originating from isomer B in step 2, 270 mg, 0.3804 mmol) was subjected to SFC using the following method: Normal phase SFC-MS method using an AS-H column (250×21.2 mm, 5 μm particle size) sold by Chiral Technologies (pn: 20945), and a dual gradient run from 15-45% mobile phase B over 14.5 minutes (includes 45-80% mobile phase rinsate). Mobile phase A=CO₂. Mobile phase B=MeOH (20 mM NH₃). Flow rate=15-45% MeOH [20 mM NH₃] 60 mL/min, 45-80% MeOH [20 mM NH₃] 40 mL/min, injection volume=variable, column temperature=40° C. Two enantiomers 1 and 2 were separated:

Enantiomer 1 was further purified by silica gel chromatography using a 12 g column eluting with 100% dichloromethane to 10% methanol in dichloromethane to afford a white solid, 12,12-dimethyl-15-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 1 (98.2 mg, 73%). ¹H NMR (400 MHz, DMSO-d₆) δ 12.63 (s, 1H), 8.18 (d, J=2.8 Hz, 1H), 7.77 (d, J=8.2 Hz, 1H), 7.62 (t, J=7.9 Hz, 1H), 7.36-7.20 (m, 6H), 7.17 (d, J=7.2 Hz, 1H), 6.92 (d, J=8.2 Hz, 1H), 6.79 (d, J=8.6 Hz, 1H), 6.08 (d, J=2.8 Hz, 1H), 4.31 (t, J=7.1 Hz, 2H), 3.71 (d, J=11.5 Hz, 1H), 3.40 (s, 1H), 2.94 (s, 1H), 2.83 (s, 1H), 2.58 (s, 2H), 2.52 (s, 1H), 2.11-2.04 (m, 2H), 1.67 (s, 1H), 1.49 (s, 3H), 1.43 (s, 3H), 1.38 (t, J=11.9 Hz, 1H), 1.22 (d, J=12.6 Hz, 1H), 0.95 (d, J=4.6 Hz, 2H), 0.90 (d, J=11.0 Hz, 2H). ESI-MS m/z calc. 709.2658, found 710.2 (M+1)⁺; Retention time: 1.84 minutes (LC method A).

Enantiomer 2 was further purified by silica gel chromatography using a 12 g column eluting with 100% dichloromethane to 10% methanol in dichloromethane to afford a white solid: 12,12-dimethyl-15-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 2 (91.3 mg, 68%). ¹H NMR (400 MHz, DMSO-d₆) δ 12.63 (s, 1H), 8.18 (d, J=2.8 Hz, 1H), 7.77 (d, J=8.2 Hz, 1H), 7.62 (t, J=7.8 Hz, 1H), 7.36-7.20 (m, 6H), 7.17 (d, J=7.2 Hz, 1H), 6.93 (d, J=8.2 Hz, 1H), 6.79 (d, J=8.5 Hz, 1H), 6.08 (d, J=2.7 Hz, 1H), 4.31 (t, J=7.0 Hz, 2H), 3.70 (s, 1H), 3.40 (s, 1H), 2.95 (s, 1H), 2.83 (s, 1H), 2.58 (s, 2H), 2.52 (d, J=1.6 Hz, 1H), 2.07 (d, J=1.5 Hz, 2H), 1.68 (s, 1H), 1.49 (s, 3H), 1.43 (s, 3H), 1.37 (d, J=12.2 Hz, 1H), 1.23 (s, 1H), 0.98-0.92 (m, 2H), 0.88 (td, J=5.9, 5.3, 3.5 Hz, 2H). ESI-MS m/z calc. 709.2658, found 710.2 (M+1)⁺; Retention time: 1.84 minutes (LC method A).

The following compounds were prepared in a manner analogous to that described above, using the isomer A of step 2 of tert-butyl N-[(3Z)-3-[1-[(4-methoxyphenyl)methyl]-5,5-dimethyl-2-oxo-pyrrolidin-3-ylidene]-3-phenyl-propyl]carbamate from step 2 as a starting material.

### Step 12: 12,12-Dimethyl-15-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 386 (enantiomer 3), and 12,12-dimethyl-15-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 385 (enantiomer 4)

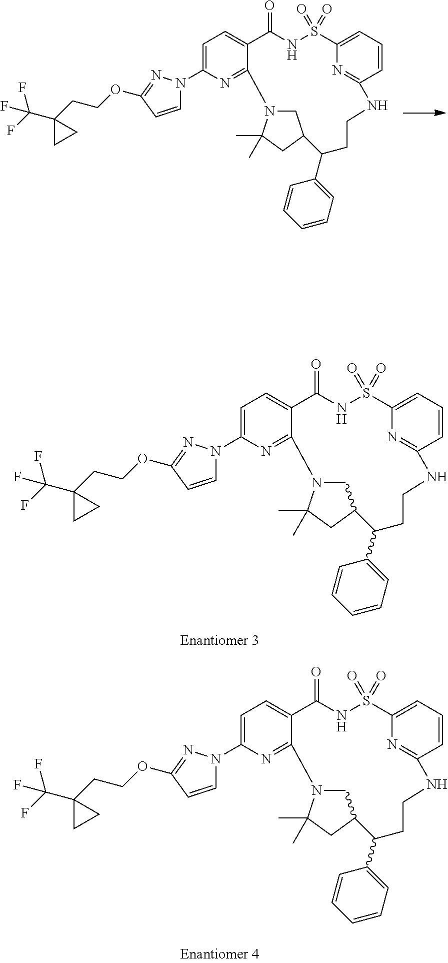 [SMILES: CC1(C)CC2CN1c1nc(-n3ccc(OCCC4(C(F)(F)F)CC4)n3)ccc1C(=O)NS(=O)(=O)c1cccc(n1)NCCC2c1ccccc1 ; CC1(C)CC2CN1c1nc(-n3ccc(OCCC4(C(F)(F)F)CC4)n3)ccc1C(=O)NS(=O)(=O)c1cccc(n1)NCCC2c1ccccc1 ; CC1(C)CC2CN1c1nc(-n3ccc(OCCC4(C(F)(F)F)CC4)n3)ccc1C(=O)NS(=O)(=O)c1cccc(n1)NCCC2c1ccccc1]

12,12-Dimethyl-15-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (mixture of two enantiomers originating from isomer A of step 2, 235 mg, 0.3311 mmol) was subjected to SFC using the following method: normal phase SFC-MS method using a LUX-4 column (250×21.2 mm, 5 μm particle size) sold by Phenomenex (pn: 00G-4491-PO-AX), and a dual gradient run from 50-80% mobile phase B over 14.5 minutes. Mobile phase A=CO₂. Mobile phase B=MeOH (20 mM NH₃). Flow rate=50-80% MeOH [20 mM NH₃] 40 mL/min. Injection volume=variable, and column temperature=40° C. Two enantiomers 3 and 4 were separated:

SFC Peak 1 (enantiomer 3) was further purified by reverse-phase preparative chromatography utilizing a C₁₈ column and a 30-99% gradient over 15 min of acetonitrile in water (+5 mM HCl) to afford a white solid:12,12-dimethyl-15-phenyl-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 3 (63.79 mg, 54%). ¹H NMR (400 MHz, DMSO-d₆) δ 12.37 (s, 1H), 8.19 (d, J=2.8 Hz, 1H), 7.62 (t, J=7.8 Hz, 2H), 7.42-7.17 (m, 7H), 6.90 (d, J=8.2 Hz, 1H), 6.78 (d, J=8.5 Hz, 1H), 6.09 (d, J=2.8 Hz, 1H), 4.30 (t, J=7.1 Hz, 2H), 3.75 (s, 1H), 3.44 (s, 1H), 3.12 (d, J=42.0 Hz, 1H), 2.89 (d, J=47.8 Hz, 3H), 2.54 (d, J=12.3 Hz, 1H), 2.09-2.04 (m, 2H), 1.81 (s, 2H), 1.66 (s, 3H), 1.57 (s, 1H), 1.38 (s, 3H), 1.00-0.92 (m, 2H), 0.91-0.84 (m, 2H). ESI-MS m/z calc. 709.2658, found 710.2 (M+1)⁺; Retention time: 1.87 minutes (LC method A).

SFC Peak 2 (enantiomer 4) was further purified by reverse-phase preparative chromatography utilizing a C₁₈ column and a 130-99% gradient over 15 min of acetonitrile in water (+5 mmolar HCl) to afford as a white solid 12,12-dimethyl-15-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 4 (50.21 mg, 43%). ¹H NMR (400 MHz, DMSO-d₆) δ 12.37 (s, 1H), 8.19 (d, J=2.7 Hz, 1H), 7.62 (t, J=8.0 Hz, 2H), 7.43-7.17 (m, 7H), 6.90 (d, J=8.2 Hz, 1H), 6.78 (d, J=8.5 Hz, 1H), 6.09 (d, J=2.8 Hz, 1H), 4.30 (t, J=7.1 Hz, 2H), 3.75 (s, 1H), 3.41 (d, J=20.2 Hz, 1H), 3.10 (s, 1H), 2.89 (d, J=45.7 Hz, 3H), 2.56-2.52 (m, 1H), 2.10-2.04 (m, 2H), 1.81 (s, 2H), 1.66 (s, 3H), 1.58 (s, 1H), 1.38 (s, 3H), 0.99-0.92 (m, 2H), 0.91-0.83 (m, 2H). ESI-MS m/z calc. 709.2658, found 710.2 (M+1)⁺; Retention time: 1.87 minutes (LC method A).

### Example 128: Preparation of 12,12-dimethyl-16-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 392 (enantiomer 1), Compound 391 (enantiomer 2), Compound 390 (enantiomer 3), and Compound 389 (enantiomer 4)

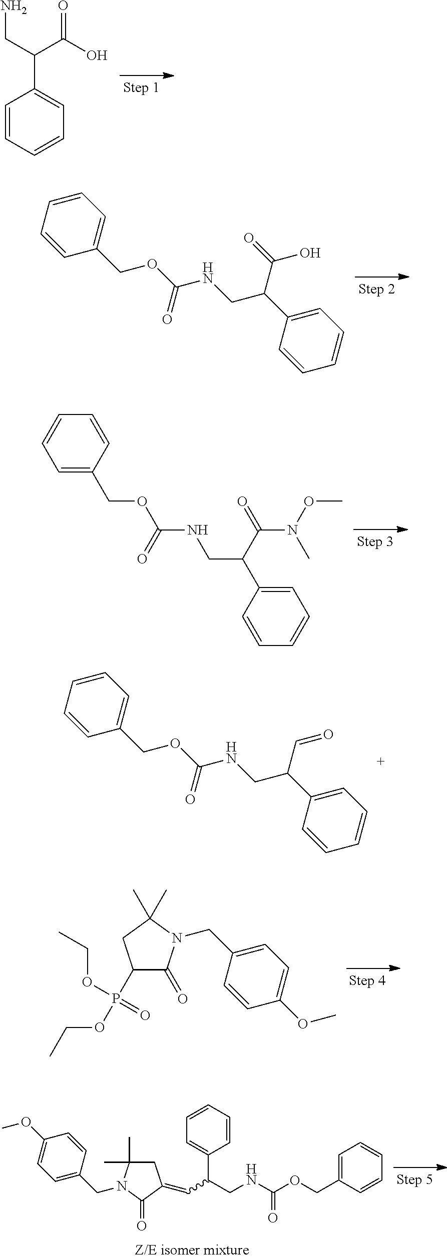

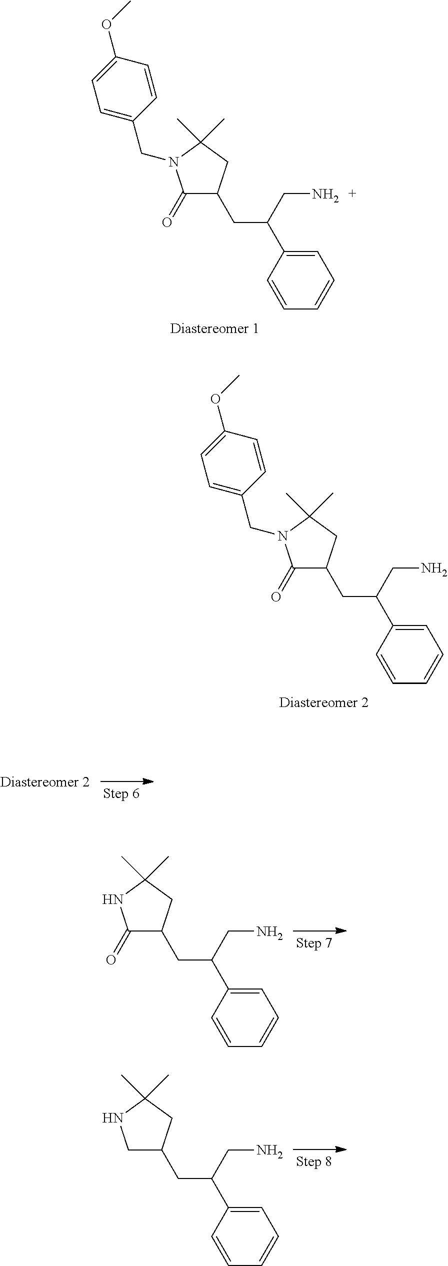 [SMILES: COc1ccc(CN2C(=O)C(CC(CN)c3ccccc3)CC2(C)C)cc1 ; COc1ccc(CN2C(=O)C(CC(CN)c3ccccc3)CC2(C)C)cc1 ; CC1(C)CC(CC(CN)c2ccccc2)C(=O)N1 ; CC1(C)CC(CC(CN)c2ccccc2)CN1]

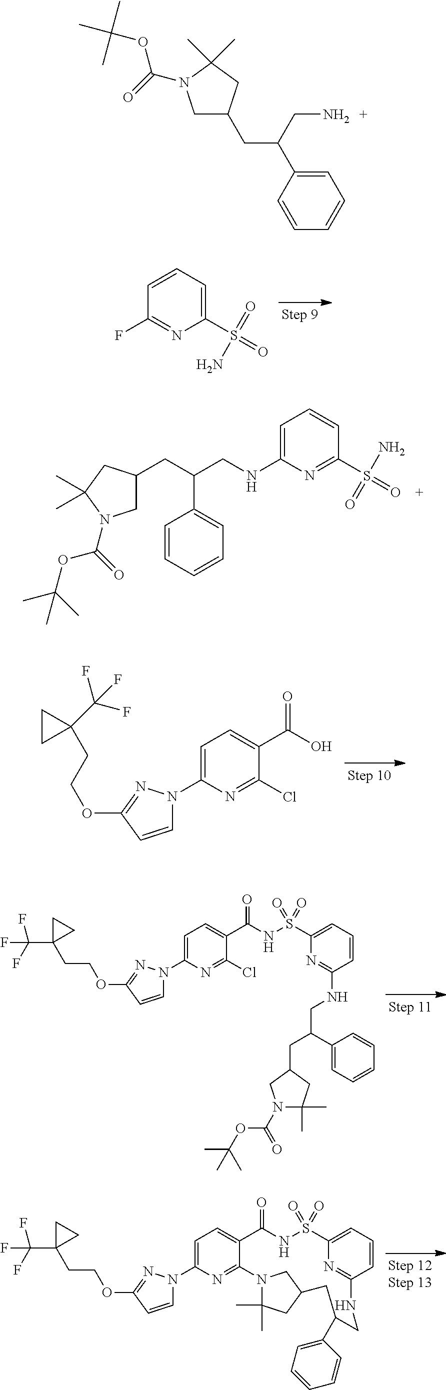 [SMILES: CC(C)(C)OC(=O)N1CC(CC(CN)c2ccccc2)CC1(C)C ; NS(=O)(=O)c1cccc(F)n1 ; CC(C)(C)OC(=O)N1CC(CC(CNc2cccc(S(N)(=O)=O)n2)c2ccccc2)CC1(C)C ; O=C(O)c1ccc(-n2ccc(OCCC3(C(F)(F)F)CC3)n2)nc1Cl ; CC(C)(C)OC(=O)N1CC(CC(CNc2cccc(S(=O)(=O)NC(=O)c3ccc(-n4ccc(OCCC5(C(F)(F)F)CC5)n4)nc3Cl)n2)c2ccccc2)CC1(C)C ; CC1(C)CC2CC(c3ccccc3)CNc3cccc(n3)S(=O)(=O)NC(=O)c3ccc(-n4ccc(OCCC5(C(F)(F)F)CC5)n4)nc3N1C2]

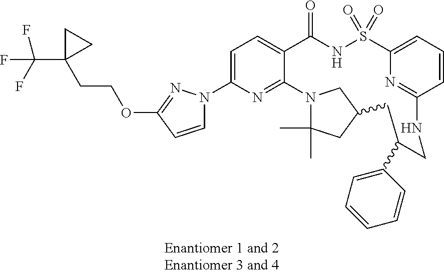 [SMILES: CC1(C)CC2CC(c3ccccc3)CNc3cccc(n3)S(=O)(=O)NC(=O)c3ccc(-n4ccc(OCCC5(C(F)(F)F)CC5)n4)nc3N1C2]

### Step 1: 3-(Benzyloxycarbonylamino)-2-phenyl-propanoic acid

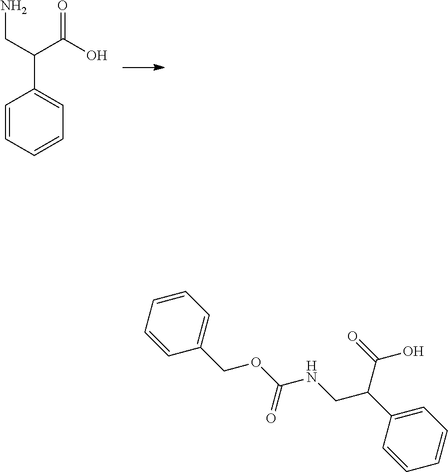 [SMILES: NCC(C(=O)O)c1ccccc1 ; O=C(NCC(C(=O)O)c1ccccc1)OCc1ccccc1]

To a solution of 3-amino-2-phenyl-propanoic acid (hydrochloride salt) (16.4 g, 81.33 mmol) in a mixture of NaHCO₃ saturated solution (420 mL) and 1,4-dioxane (420 mL) at 0° C. was added CbzCl (14 mL, 93.53 mmol) dropwise, then the reaction solution was stirred for 19 hours while the reaction was allowed to warm up to ambient temperature. The reaction solution was acidified with an HCl aqueous solution (6M) until pH reached 2. Most of 1,4-dioxane was removed under reduced pressure. The solution was extracted with ethyl acetate (3×400 mL). The combined organic layers were dried over sodium sulfate, filtered and concentrated under reduced pressure to afford a pale yellow liquid of 3-(benzyloxycarbonylamino)-2-phenyl-propanoic acid (27.83 g, 95%). ESI-MS m/z calc. 299.1158, found 300.2 (M+1)⁺; Retention time: 4.31 minutes (LC method C).

### Step 2: Benzyl N-[3-[methoxy(methyl)amino]-3-oxo-2-phenyl-propyl]carbamate

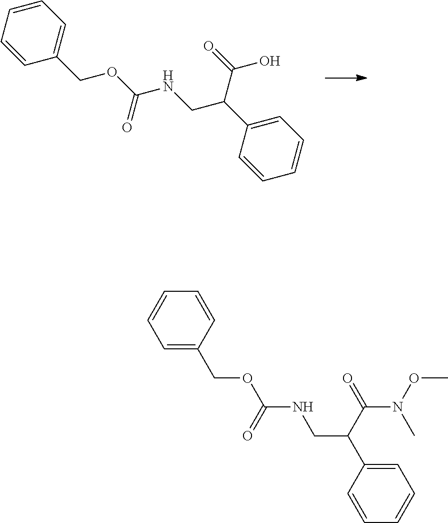 [SMILES: O=C(NCC(C(=O)O)c1ccccc1)OCc1ccccc1 ; CON(C)C(=O)C(CNC(=O)OCc1ccccc1)c1ccccc1]

To a solution of 3-(benzyloxycarbonylamino)-2-phenyl-propanoic acid (27.83 g, 77.17 mmol) and N,O-Dimethylhydroxylamine (hydrochloride salt) (9.033 g, 92.61 mmol) in anhydrous CH₂Cl₂ (300 mL) at 0° C. was added Et₃N (12.9 mL, 92.605 mmol) followed by EDCI (17.752 g, 92.605 mmol). The resulting solution was stirred for 2 days while the reaction was allowed to warm up to ambient temperature. To the reaction solution was added dichloromethane (500 mL) and brine (80 mL). The organic layer was separated, washed with an HCl aqueous solution (80 mL, 1M), saturated sodium bicarbonate aqueous solution (80 mL) and brine (80 mL). The organic layer was then dried over anhydrous sodium sulfate, filtered and concentrated under reduced pressure. The crude product was purified by flash chromatography (loaded in CH₂Cl₂) (330 g SiO₂, eluting 0 to 100% ethyl ether/hexanes), product fractions were combined and concentrated to afford a colorless oil of benzyl N-[3-[methoxy(methyl)amino]-3-oxo-2-phenyl-propyl]carbamate (23.7 g, 90%). ¹1HNMR (250 MHz, CDCl₃) δ 7.52-7.05 (m, 10H), 5.39-5.20 (m, 1H), 5.09 (s, 2H), 4.41-4.13 (m, 1H), 3.60 (t, J=6.9 Hz, 2H), 3.28 (s, 3H), 3.15 (s, 3H). ESI-MS m/z calc. 342.158, found 343.3 (M+1)⁺; Retention time: 4.76 minute, (LC method C).

### Step 3: Benzyl N-(3-oxo-2-phenyl-propyl)carbamate

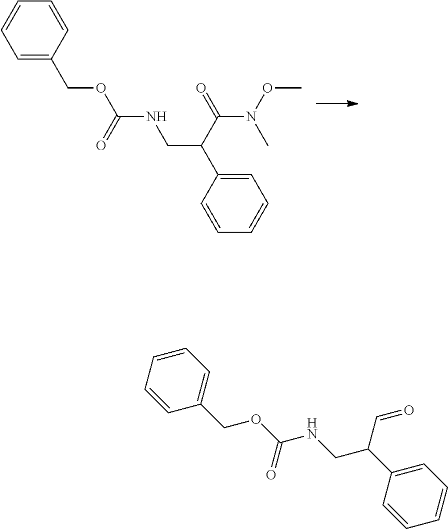 [SMILES: CON(C)C(=O)C(CNC(=O)OCc1ccccc1)c1ccccc1 ; O=CC(CNC(=O)OCc1ccccc1)c1ccccc1]

To a solution of benzyl N-[3-[methoxy(methyl)amino]-3-oxo-2-phenyl-propyl]carbamate (23.7 g, 69.22 mmol) in anhydrous THF (350 mL) at −78° C. was added LAH (3.34 g, 88.00 mmol). The reaction solution was stirred for 2 hours while the reaction solution warmed up to 0° C. before the reaction was re-cooled to −78° C. Saturated Rochelle salt aqueous solution (200 mL) was added very slowly. The reaction solution was allowed to warm-up to ambient temperature and stirred further for 1 hour until reaction solution turned into two clear layers. The organic layer was separated, and the aqueous layer was extracted with diethyl ether (2×250 mL). The combined organic layers were washed with brine (2×200 mL), dried over anhydrous sodium sulfate, filtered and concentrated under reduced pressure. The crude product was purified by flash chromatography (loaded in CH₂Cl₂) (330 g SiO₂, eluting 0 to 70% ethyl ether/hexanes), concentrated to afford a colorless oil of benzyl N-(3-oxo-2-phenyl-propyl)carbamate (16.01 g, 82%). ¹1HNMR (250 MHz, CDCl₃) δ 9.72 (s, 1H), 7.67-7.01 (m, 10H), 5.33-4.88 (m, 3H), 3.92 (dd, J=8.2, 5.8 Hz, 1H), 3.73 (ddd, J=13.9, 8.1, 5.8 Hz, 1H), 3.58 (dt, J=13.7, 6.3 Hz, 1H). ESI-MS m/z calc. 283.1208, found 284.3 (M+1)⁺; Retention time: 4.58 minutes (LC method C).

### Step 4: Benzyl N-[(3E)-3-[1-[(4-methoxyphenyl)methyl]-5,5-dimethyl-2-oxo-pyrrolidin-3-ylidene]-2-phenyl-propyl]carbamate and benzyl N-[(3Z)-3-[1-[(4-methoxyphenyl)methyl]-5,5-dimethyl-2-oxo-pyrrolidin-3-ylidene]-2-phenyl-propyl]carbamate

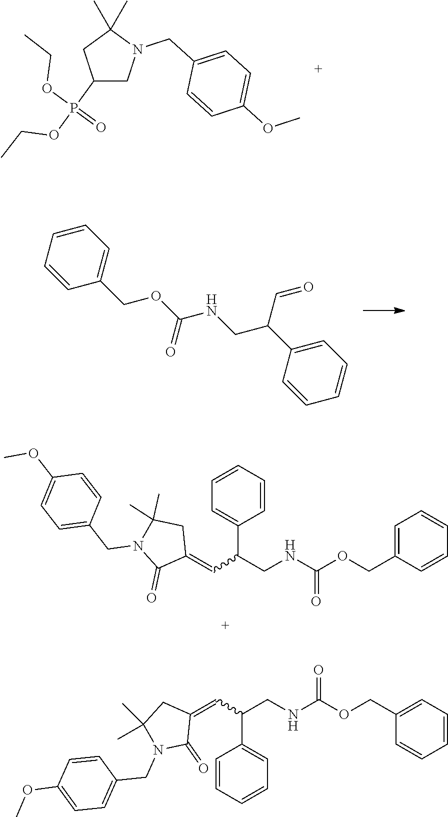

To a solution of 3-diethoxyphosphoryl-1-[(4-methoxyphenyl)methyl]-5,5-dimethyl-pyrrolidin-2-one (18.09 g, 48.97 mmol) in anhydrous THF (300 mL) at 0° C. was added NaH (2.364 g, 59.11 mmol) under argon. The resulting solution was stirred at this temperature for 30 minutes, then benzyl N-(3-oxo-2-phenyl-propyl)carbamate (16 g, 53.65 mmol) in anhydrous THF (150 mL) was added slowly. The resulting solution was stirred for 2 days at ambient temperature. To the reaction solution was added water (300 mL) and the aqueous phase was extracted with ethyl acetate (3×300 mL). The combined organic layer was washed with brine (200 mL), dried over sodium sulfate, filtered and concentrated under reduced pressure. The crude product was purified by flash chromatography (loaded in CH₂Cl₂) (330 g SiO₂, eluting 0 to 80% ethyl ether/hexanes), to afford two isomers (Z/E stereochemistry arbitrarily assigned):

A white foam solid of benzyl N-[(3E)-3-[1-[(4-methoxyphenyl)methyl]-5,5-dimethyl-2-oxo-pyrrolidin-3-ylidene]-2-phenyl-propyl]carbamate (10.73 g, 44%). ¹1HNMR (250 MHz, CDCl₃) δ 7.49-7.27 (m, 8H), 7.26-7.17 (m, 4H), 6.81 (dd, J=9.1, 2.5 Hz, 2H), 6.76-6.63 (m, 1H), 5.09 (s, 2H), 4.84 (s, 1H), 4.57-4.31 (m, 2H), 3.84-3.78 (m, 1H), 3.77 (s, 3H), 3.60-3.37 (m, 2H), 2.49 (q, J=16.7 Hz, 2H), 1.12 (s, 3H), 1.08 (s, 3H). ESI-MS m/z calc. 498.2519, found 499.4 (M+1)⁺; Retention time: 5.84 minutes (LC method C).

A pale yellow sticky oil of benzyl N-[(3Z)-3-[1-[(4-methoxyphenyl)methyl]-5,5-dimethyl-2-oxo-pyrrolidin-3-ylidene]-2-phenyl-propyl]carbamate (9.95 g, 40%). ¹1HNMR (250 MHz, CDCl₃) δ 7.34 (h, J=2.9 Hz, 10H), 7.26-7.18 (m, 2H), 6.77 (d, J 8.6 Hz, 2H), 5.98 (d, J=10.2 Hz, 1H), 5.72 (d, J=7.3 Hz, 1H), 5.23 (td, J=10.3, 5.6 Hz, 1H), 5.16-4.96 (m, 2H), 4.51 (d, J=15.3 Hz, 1H), 4.32 (d, J=15.2 Hz, 1H), 3.75 (s, 3H), 3.72-3.55 (m, 1H), 3.52-3.30 (m, 1H), 2.48 (s, 2H), 1.10 (s, 6H). ESI-MS m/z calc. 498.2519, found 499.5 (M+1)⁺; Retention time: 6.2 minutes ((LC method C).

### Step 5: 3-(3-Amino-2-phenyl-propyl)-1-[(4-methoxyphenyl)methyl]-5,5-dimethyl-pyrrolidin-2-one, diastereomer 1 and 3-(3-amino-2-phenyl-propyl)-1-[(4-methoxyphenyl)methyl]-5,5-dimethyl-pyrrolidin-2-one, diastereomer 2

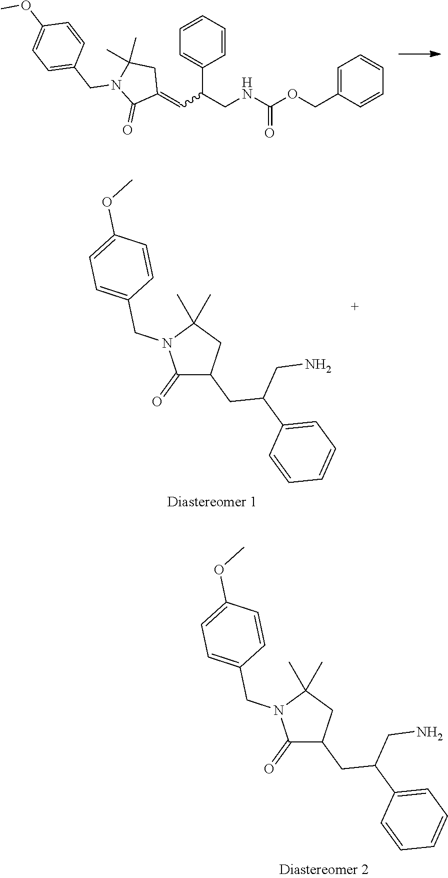 [SMILES: COc1ccc(CN2C(=O)C(=CC(CNC(=O)OCc3ccccc3)c3ccccc3)CC2(C)C)cc1 ; COc1ccc(CN2C(=O)C(CC(CN)c3ccccc3)CC2(C)C)cc1 ; COc1ccc(CN2C(=O)C(CC(CN)c3ccccc3)CC2(C)C)cc1]

To a solution of benzyl N-[3-[1-[(4-methoxyphenyl)methyl]-5,5-dimethyl-2-oxo-pyrrolidin-3-ylidene]-2-phenyl-propyl]carbamate (20.97 g, 42.06 mmol) (recombined mixture of Z and E isomers) in anhydrous EtOH (500 mL) was added 10% Pd/C (2 g, 18.79 mmol). The resulting solution was hydrogenated under 60 PSI in a Parr Shake for 44 hours. The reaction solution was filtered through Celite and washed with ethanol. The filtrate was concentrated under reduced pressure to afford a crude product (15.5 g sticky oil) with two stereoisomers in a ratio of 1:1.4. The crude product was purified with reverse HPLC (2.5 g/run, total 6 runs) using a gradient of 20 to 55% mobile phase B over 60 minutes (mobile phase A: water with 0.1% TFA; mobile phase B: acetonitrile with 0.1% TFA). The pure fractions for each isomer were combined and concentrated under reduced pressure to remove acetonitrile. The aqueous layer was then basified carefully with solid sodium carbonate (to reach pH=9 to 10) and extracted with ethyl acetate. The combined ethyl acetate layer was dried over anhydrous sodium sulfate, filtered and concentrated under reduced pressure to afford two pure isomers.

Diastereomer 1 (sticky pale yellow oil, more polar):3-(3-amino-2-phenyl-propyl)-1-[(4-methoxyphenyl)methyl]-5,5-dimethyl-pyrrolidin-2-one (4.636 g, 30%). ¹1HNMR (250 MHz, CDCl₃) δ 7.48-7.07 (m, 7H), 6.79 (d, J=8.7 Hz, 2H), 4.51-4.15 (m, 2H), 3.77 (s, 3H), 2.92 (d, J=6.8 Hz, 2H), 2.76-2.53 (m, 1H), 2.39 (td, J=12.7, 12.0, 3.6 Hz, 1H), 2.31-2.09 (m, 1H), 2.09-1.91 (m, 1H), 1.69-1.58 (m, 1H), 1.48 (s, 2H), 1.27 (d, J=2.8 Hz, 1H), 1.16 (s, 3H), 0.91 (s, 3H). ESI-MS m/z calc. 366.2307, found 367.2 (M+1)⁺; Retention time: 3.38 minutes (LC method C).

Diastereomer 2 (sticky pale yellow oil, less polar): 3-(3-amino-2-phenyl-propyl)-1-[(4-methoxyphenyl)methyl]-5,5-dimethyl-pyrrolidin-2-one (7.3 g, 47%). ¹1HNMR (250 MHz, CDCl₃) δ 7.49-7.04 (m, 7H), 6.91-6.66 (m, 2H), 4.42 (d, J=15.2 Hz, 1H), 4.21 (d, J=15.2 Hz, 1H), 3.77 (s, 3H), 2.94 (td, J=16.0, 14.2, 10.5 Hz, 3H), 2.58-2.24 (m, 2H), 1.76 (dd, J=12.4, 8.1 Hz, 1H), 1.62 (s, 2H), 1.37-1.17 (m, 2H), 1.07 (s, 3H), 0.96 (s, 3H). ESI-MS m/z calc. 366.2307, found 367.2 (M+1)⁺; Retention time: 3.67 minutes (LC method C).

### Step 6: 3-(3-Amino-2-phenyl-propyl)-5,5-dimethyl-pyrrolidin-2-one

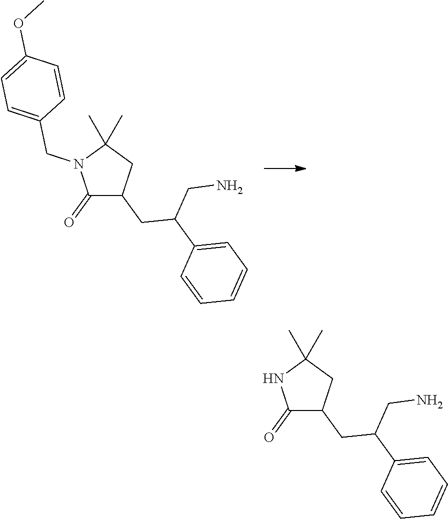 [SMILES: COc1ccc(CN2C(=O)C(CC(CN)c3ccccc3)CC2(C)C)cc1 ; CC1(C)CC(CC(CN)c2ccccc2)C(=O)N1]

3-(3-Amino-2-phenyl-propyl)-1-[(4-methoxyphenyl)methyl]-5,5-dimethyl-pyrrolidin-2-one, less polar diastereomer 2 (7.29 g, 19.69 mmol) was dissolved in TFA (110 mL, 1.43 mol) and was heated at 100° C. in a sealed tube for 69 hours. 1,2-Dichloromethane (50 mL) was added, and all solvents were removed under reduced pressure. The crude product was purified with reverse HPLC (1.5 g/run, total 2 runs) using gradient of 5 to 45% mobile phase B in 40 minutes (mobile phase A: water with 0.1% TFA; mobile phase B: acetonitrile with 0.1% TFA). The pure fractions were combined and concentrated under reduced pressure to remove most of solvents. The aqueous layer was then basified carefully with solid sodium carbonate (pH=9 to 10) and extracted with 2-methyl tetrahydrofuran. The organic layer was dried over anhydrous sodium sulfate, filtered and concentrated under reduced pressure to afford a white foam of 3-(3-amino-2-phenyl-propyl)-5,5-dimethyl-pyrrolidin-2-one (5.10 g, 100%). ¹H NMR (250 MHz, CDCl₃) δ 7.45-7.11 (m, 5H), 6.14 (s, 1H), 3.35-3.08 (m, 3H), 3.08-2.85 (m, 3H), 2.61-2.12 (m, 2H), 1.76 (dd, J=12.6, 8.4 Hz, 1H), 1.53 (dd, J=14.4, 7.5 Hz, 1H), 1.19 (s, 3H), 1.12 (s, 3H). ESI-MS m/z calc. 246.1732, found 247.1 (M+1)⁺; Retention time: 2.27 minutes (LC method C).

### Step 7: 3-(5,5-Dimethylpyrrolidin-3-yl)-2-phenyl-propan-1-amine

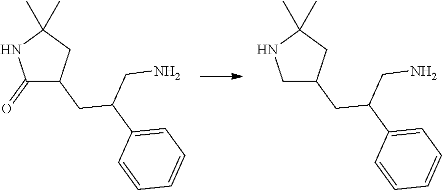 [SMILES: CC1(C)CC(CC(CN)c2ccccc2)C(=O)N1 ; CC1(C)CC(CC(CN)c2ccccc2)CN1]

To a solution of 3-(3-amino-2-phenyl-propyl)-5,5-dimethyl-pyrrolidin-2-one (1.596 g, 6.15 mmol) in anhydrous THF (65 mL) was added LAH (2.05 g, 54.012 mmol) slowly. The resulting solution was stirred at 40° C. under argon for 2 days, and then refluxed for 2 days. The reaction solution was cooled to 0° C., then water (2.1 mL) was added slowly followed by 15% NaOH aqueous solution (2.1 mL) and water (6.3 mL). The solution was stirred for 30 minutes at ambient temperature, then filtered through Celite and washed with THF. The filtrate was concentrated under reduced pressure to afford 3-(5,5-dimethylpyrrolidin-3-yl)-2-phenyl-propan-1-amine (1.529 g, 89%) as a pale sticky oil. ESI-MS m/z calc. 232.1939, found 233.3 (M+1)⁺; Retention time: 1.43 minutes, 1.31 minutes (LC method C).

### Step 8: tert-Butyl 4-(3-amino-2-phenyl-propyl)-2,2-dimethyl-pyrrolidine-1-carboxylate

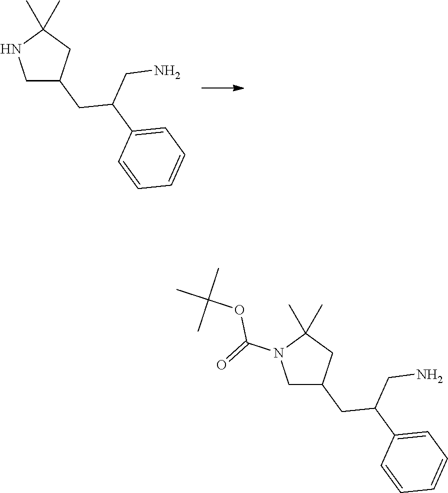 [SMILES: CC1(C)CC(CC(CN)c2ccccc2)CN1 ; CC(C)(C)OC(=O)N1CC(CC(CN)c2ccccc2)CC1(C)C]

In a flask equipped with a Dean-Stark trap and a condenser, a mixture of 3-(5,5-dimethylpyrrolidin-3-yl)-2-phenyl-propan-1-amine (1.394 g, 4.98 mmol) in methyl isobutyl ketone (40 mL) was heated to reflux under argon for about 10 hours. The reaction solution cooled to 0° C., then Boc₂O (1.118 g, 1.1768 mL, 5.1226 mmol) in methyl isobutyl ketone (4 mL) was added dropwise. After the addition was finished, the resulting solution was stirred at ambient temperature for 30 minutes. Then water (10 mL) was added, the organic layer was separated. The organic layer was concentrated under reduced pressure. The residue obtained was dissolved in a mixture of water (4 mL) and 2-propanol (40 mL) and heated at 50° C. for 2 hours until complete conversion of imine to primary amine. All solvents were removed under reduced pressure. To the residue was added toluene (2×10 mL) and toluene was removed under reduced pressure to afford a crude product of tert-butyl 4-(3-amino-2-phenyl-propyl)-2,2-dimethyl-pyrrolidine-1-carboxylate (1.86 g, 56%) as a sticky brown oil. ESI-MS m/z calc. 332.2464, found 333.4 (M+1)⁺; Retention time: 3.95 minutes (LC method C).

### Step 9: tert-Butyl 2,2-dimethyl-4-[2-phenyl-3-[(6-sulfamoyl-2-pyridyl)amino]propyl]pyrrolidine-1-carboxylate

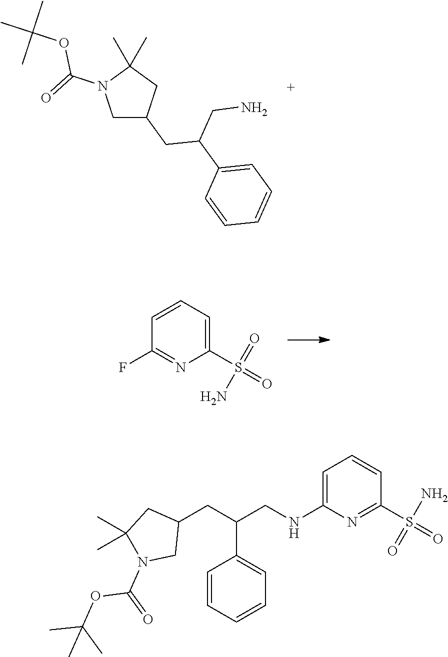 [SMILES: CC(C)(C)OC(=O)N1CC(CC(CN)c2ccccc2)CC1(C)C ; NS(=O)(=O)c1cccc(F)n1 ; CC(C)(C)OC(=O)N1CC(CC(CNc2cccc(S(N)(=O)=O)n2)c2ccccc2)CC1(C)C]

To a mixture of tert-butyl 4-(3-amino-2-phenyl-propyl)-2,2-dimethyl-pyrrolidine-1-carboxylate (1.605 g, 2.41 mmol) and 6-fluoropyridine-2-sulfonamide (563 mg, 3.20 mmol) in anhydrous DMSO (4.8 mL) was added K₂CO₃ (1.18 g, 8.54 mmol). The resulting solution was stirred at 100° C. under argon for 34 hours. The reaction solution was partitioned between ethyl acetate (150 mL) and citric acid (28.5 mL, 0.3M). The organic layer was separated, washed with brine, dried over anhydrous sodium sulfate, filtered and concentrated under reduced pressure. The crude product was purified by flash chromatography (loaded in CH₂Cl₂) (40 g SiO₂, eluting 0 to 70% ethyl ether/hexanes) to afford brown solid foam of tert-Butyl 2,2-dimethyl-4-[2-phenyl-3-[(6-sulfamoyl-2-pyridyl)amino]propyl]pyrrolidine-1-carboxylate (291 mg, 25%). ¹H NMR (250 MHz, DMSO-d₆) δ 7.50 (t, J=7.8 Hz, 1H), 7.34-7.26 (m, 3H), 7.26-7.17 (m, 1H), 7.10 (s, 2H), 7.00-6.86 (m, 2H), 6.60 (d, J=8.8 Hz, 1H), 3.58-3.42 (m, 3H), 2.92-2.74 (m, 2H), 1.88-1.58 (m, 5H), 1.37 (s, 9H), 1.29 (d, J=4.5 Hz, 3H), 1.09 (s, 3H). ESI-MS m/z calc. 488.2457, found 489.4 (M+1)⁺; Retention time: 2.73 minutes (LC method H).

### Step 10: tert-Butyl 4-[3-[[6-[[2-chloro-6-[3-[2-[1-(trifluoromethyl)cyclopropyl] ethoxy]pyrazol-1-yl]pyridine-3-carbonyl]sulfamoyl]-2-pyridyl]amino]-2-phenyl-propyl]-2,2-dimethyl-pyrrolidine-1-carboxylate

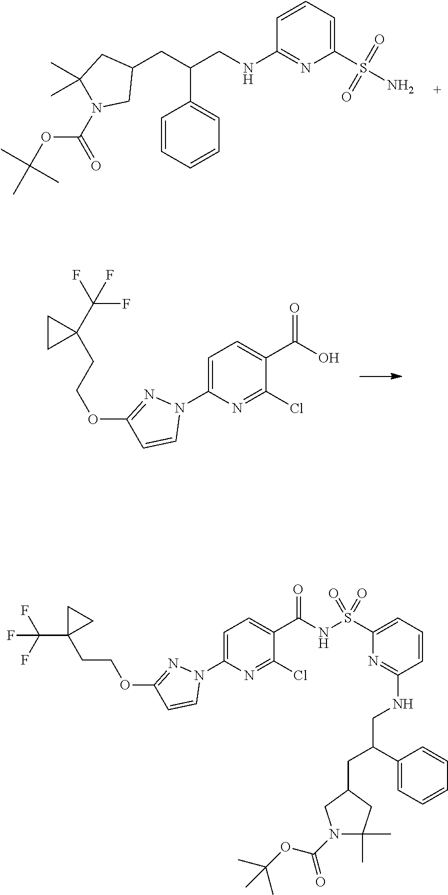 [SMILES: CC(C)(C)OC(=O)N1CC(CC(CNc2cccc(S(N)(=O)=O)n2)c2ccccc2)CC1(C)C ; O=C(O)c1ccc(-n2ccc(OCCC3(C(F)(F)F)CC3)n2)nc1Cl ; CC(C)(C)OC(=O)N1CC(CC(CNc2cccc(S(=O)(=O)NC(=O)c3ccc(-n4ccc(OCCC5(C(F)(F)F)CC5)n4)nc3Cl)n2)c2ccccc2)CC1(C)C]

2-Chloro-6-[3-[2-[1-(trifluoromethyl)cyclopropyl]ethoxy]pyrazol-1-yl]pyridine-3-carboxylic acid (324 mg, 0.86 mmol) and carbonyl diimidazole (153 mg, 0.9436 mmol) were combined in THF (2 mL) and stirred for 2 h at rt. Then tert-butyl 2,2-dimethyl-4-[2-phenyl-3-[(6-sulfamoyl-2-pyridyl)amino]propyl]pyrrolidine-1-carboxylate (275 mg, 0.56 mmol) was added followed by DBU (500 μL, 3.343 mmol) and the reaction was stirred at rt for 14 h. The reaction mixture was quenched with saturated ammonium chloride and extracted with ethyl acetate. The combined organic layers were washed with brine, dried (MgSO₄), filtered and concentrated. The resulting brown residue was purified by silica gel column chromatography using a shallow gradient 100% hexanes to 100% EtOAc to afford tert-butyl 4-[3-[[6-[[2-chloro-6-[3-[2-[1-(trifluoromethyl)cyclopropyl] ethoxy]pyrazol-1-yl]pyridine-3-carbonyl]sulfamoyl]-2-pyridyl]amino]-2-phenyl-propyl]-2,2-dimethyl-pyrrolidine-1-carboxylate (229 mg, 48%) as an off white solid. ESI-MS m/z calc. 845.2949, found 846.2 (M+1)⁺; Retention time: 0.61 minutes (LC method U).

### Step 11: 12,12-Dimethyl-16-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione

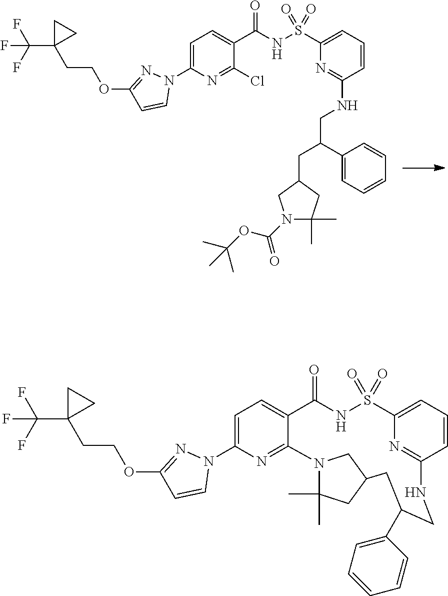 [SMILES: CC(C)(C)OC(=O)N1CC(CC(CNc2cccc(S(=O)(=O)NC(=O)c3ccc(-n4ccc(OCCC5(C(F)(F)F)CC5)n4)nc3Cl)n2)c2ccccc2)CC1(C)C ; CC1(C)CC2CC(c3ccccc3)CNc3cccc(n3)S(=O)(=O)NC(=O)c3ccc(-n4ccc(OCCC5(C(F)(F)F)CC5)n4)nc3N1C2]

A solution of tert-butyl 4-[3-[[6-[[2-chloro-6-[3-[2-[1-(trifluoromethyl) cyclopropyl]ethoxy]pyrazol-1-yl]pyridine-3-carbonyl]sulfamoyl]-2-pyridyl]amino]-2-phenyl-propyl]-2,2-dimethyl-pyrrolidine-1-carboxylate (570 mg, 0.67 mmol) in TFA (700 μL, 9.09 mmol) and DCM (2 mL) was stirred at rt for 2h. After completion of the reaction, solvents were removed. The residue was dissolved in ethyl acetate and washed with a saturated NaHCO₃ solution (2 mL) and the organic layer was collected and the solvent removed. The material was dissolved in dimethyl sulfoxide (15 mL), oven dried 4 Å molecular sieves was added and the mixture was stirred for 10 min. Then, potassium carbonate (320 mg, 2.31 mmol) was added and the reaction mixture was heated at 140° C. for 16h. The reaction mixture was filtered through a Whatman filter disc (puradisc 25 TF) and the filtrate was purified by a reverse phase HPLC-MS method using a dual gradient run from 50-99% mobile phase B over 15.0 minutes. (Mobile phase A=H₂O (5 mM HCl). Mobile phase B=CH₃CN) to afford 12,12-dimethyl-16-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (285 mg, 60%) as an off white solid. ESI-MS m/z calc. 709.2658, found 710.2 (M+1)⁺; Retention time: 1.31 minutes (LC method M).

### Step 12: 12,12-Dimethyl-16-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 392 (enantiomer 1), and 12,12-dimethyl-16-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 391 (enantiomer 2)

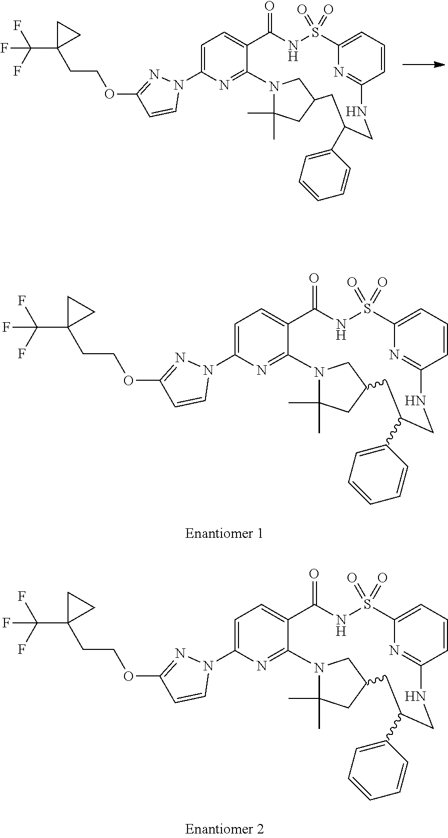 [SMILES: CC1(C)CC2CC(c3ccccc3)CNc3cccc(n3)S(=O)(=O)NC(=O)c3ccc(-n4ccc(OCCC5(C(F)(F)F)CC5)n4)nc3N1C2 ; CC1(C)CC2CC(c3ccccc3)CNc3cccc(n3)S(=O)(=O)NC(=O)c3ccc(-n4ccc(OCCC5(C(F)(F)F)CC5)n4)nc3N1C2 ; CC1(C)CC2CC(c3ccccc3)CNc3cccc(n3)S(=O)(=O)NC(=O)c3ccc(-n4ccc(OCCC5(C(F)(F)F)CC5)n4)nc3N1C2]

12,12-Dimethyl-16-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, isomeric mixture originating from 3-(3-Amino-2-phenyl-propyl)-1-[(4-methoxyphenyl)methyl]-5,5-dimethyl-pyrrolidin-2-one, less polar diastereomer 2 (285 mg, 0.4015 mmol) was subjected to chiral separation by SFC chromatography using a ChiralPak LUX-3 (250×21.2 mm), 5 μm particle size using 15% MeOH (No modifier), 85% CO₂ to 45% MeOH (no modifier), 65% CO₂) at 40 mL/min over 14 minutes, giving as the first enantiomer to elute (PEAK-1):

Enantiomer 1: 12,12-dimethyl-16-phenyl-8-(3-{2-[1-(trifluoromethyl) cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, (66.1 mg, 46%). ¹H NMR (400 MHz, Chloroform-d) δ 8.23-8.15 (m, 2H), 7.65-7.53 (m, 2H), 7.38 (d, J=8.4 Hz, 1H), 7.32 (t, J=7.4 Hz, 2H), 7.28-7.20 (m, 1H), 7.12 (dd, J=7.0, 1.8 Hz, 2H), 6.54 (d, J=7.9 Hz, 1H), 5.91 (d, J=2.8 Hz, 1H), 4.98 (s, 1H), 4.53 (t, J=11.5 Hz, 1H), 4.40 (t, J=7.1 Hz, 2H), 3.79 (t, J=8.5 Hz, 1H), 3.29 (s, 1H), 3.03 (qd, J=8.9, 8.3, 3.7 Hz, 2H), 2.46-2.27 (m, 2H), 2.09 (t, J=7.2 Hz, 2H), 2.05-1.97 (m, 1H), 1.63 (d, J=5.7 Hz, 6H), 1.59-1.47 (m, 2H), 1.04-0.97 (m, 2H), 0.75-0.70 (m, 2H). ESI-MS m/z calc. 709.2658, found 710.14 (M+1)⁺; Retention time: 1.36 minutes (LC method M).

Enantiomer 2 (second-eluting enantiomer): 12,12-dimethyl-16-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 2 (87.5 mg, 57%). ¹H NMR (400 MHz, Chloroform-d) δ 8.22-8.16 (m, 2H), 7.58 (q, J=7.8 Hz, 2H), 7.38 (d, J=8.4 Hz, 1H), 7.32 (t, J=7.4 Hz, 2H), 7.26-7.21 (m, 1H), 7.15-7.10 (m, 2H), 6.54 (d, J=7.9 Hz, 1H), 5.91 (d, J=2.8 Hz, 1H), 5.00 (d, J=14.3 Hz, 1H), 4.54 (t, J=11.7 Hz, 1H), 4.40 (t, J=7.2 Hz, 2H), 3.79 (t, J=8.4 Hz, 1H), 3.29 (s, 1H), 3.03 (qd, J=8.5, 3.8 Hz, 2H), 2.43-2.27 (m, 2H), 2.09 (t, J=7.2 Hz, 2H), 2.02 (d, J=11.7 Hz, 1H), 1.63 (d, J=6.1 Hz, 6H), 1.58-1.49 (m, 2H), 1.04-0.98 (m, 2H), 0.77-0.69 (m, 2H). ESI-MS m/z calc. 709.2658, found 710.2 (M+1)⁺; Retention time: 1.36 minutes (LC method M).

The following compounds were prepared in a manner analogous to that described above, using (3-amino-2-phenyl-propyl)-1-[(4-methoxyphenyl)methyl]-5,5-dimethyl-pyrrolidin-2-one diastereomer 1 as a starting material.

### Step 13: 12,12-Dimethyl-16-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 390 (enantiomer 3), and 12,12-dimethyl-16-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, Compound 389 (enantiomer 4)

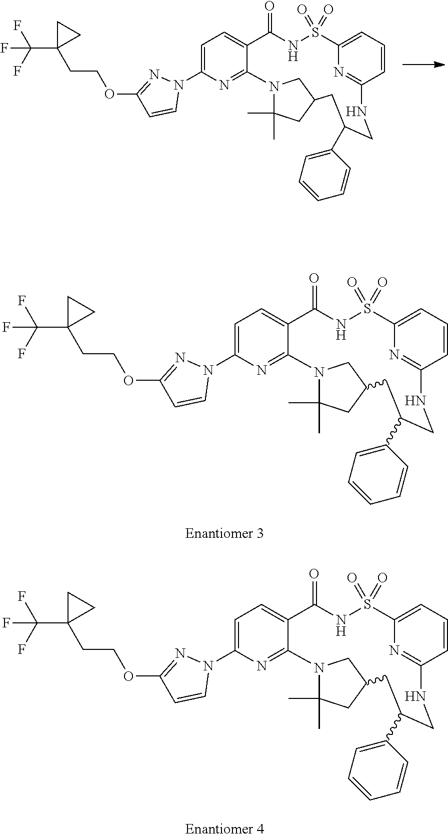 [SMILES: CC1(C)CC2CC(c3ccccc3)CNc3cccc(n3)S(=O)(=O)NC(=O)c3ccc(-n4ccc(OCCC5(C(F)(F)F)CC5)n4)nc3N1C2 ; CC1(C)CC2CC(c3ccccc3)CNc3cccc(n3)S(=O)(=O)NC(=O)c3ccc(-n4ccc(OCCC5(C(F)(F)F)CC5)n4)nc3N1C2 ; CC1(C)CC2CC(c3ccccc3)CNc3cccc(n3)S(=O)(=O)NC(=O)c3ccc(-n4ccc(OCCC5(C(F)(F)F)CC5)n4)nc3N1C2]

A first batch of 12,12-Dimethyl-16-phenyl-8-(3-{2-[1-(trifluoromethyl) cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, mixture of two isomers originating from (3-amino-2-phenyl-propyl)-1-[(4-methoxyphenyl)methyl]-5,5-dimethyl-pyrrolidin-2-one, diastereomer 1, (50 mg, 0.070 mmol) was subjected to SFC separation using the following method: ChiralPaK AS-3 (250×10 mm, 5 μm) column, 35° C., mobile phase 32% MeOH (no modifier), 68% CO₂, flow 10 mL/min, concentrations 24 mg/mL in MeOH (no modifier), injection volume 70 μL, pressure 100 bar, wavelength 210 nm.

A second batch of the same material (40 mg, 0.0564 mmol) was subjected to SFC separation using the following method: ChiralPak AS-H (250×21.2 mm), 5 μm particle size column using a gradient of 15% MeOH (no modifier)/85% CO₂ to 45% MeOH (no modifier)/65% CO₂ over 14 minutes, flow 40 mL/min.

The peak 1 products from each separation were combined to give 12,12-dimethyl-16-phenyl-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 3 (30.5 mg, 64%). ¹H NMR (400 MHz, Chloroform-d) δ 10.45 (s, 1H), 8.18 (d, J=2.8 Hz, 1H), 8.14 (d, J=8.4 Hz, 1H), 7.58 (d, J=4.3 Hz, 2H), 7.37-7.30 (m, 3H), 7.26 (t, J=3.6 Hz, 1H), 7.18-7.12 (m, 2H), 6.61-6.52 (m, 1H), 5.91 (d, J=2.8 Hz, 1H), 4.84 (s, 1H), 4.40 (t, J=7.1 Hz, 2H), 3.68 (d, J=10.5 Hz, 2H), 3.38 (d, J=29.4 Hz, 2H), 2.83 (s, 1H), 2.31 (s, 2H), 2.09 (t, J=7.2 Hz, 2H), 2.01 (dd, J=12.1, 6.9 Hz, 1H), 1.92 (s, 1H), 1.72 (t, J=11.2 Hz, 1H), 1.60 (s, 3H), 1.56 (s, 3H), 1.07-0.97 (m, 2H), 0.74 (q, J=1.7 Hz, 2H). ESI-MS m/z calc. 709.2658, found 710.2 (M+1)⁺; Retention time: 1.39 minutes, (LC method M).

The peak 2 products from each separation were combined to give 12,12-dimethyl-16-phenyl-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione enantiomer 4: (34.6 mg, 72%). ¹H NMR (400 MHz, Chloroform-d) δ 10.51 (s, 1H), 8.18 (d, J=2.8 Hz, 1H), 8.14 (d, J=8.4 Hz, 1H), 7.58 (d, J=4.8 Hz, 2H), 7.33 (q, J=8.4, 7.9 Hz, 3H), 7.26 (t, J=3.7 Hz, 1H), 7.19-7.12 (m, 2H), 6.56 (t, J=4.7 Hz, 1H), 5.91 (d, J=2.8 Hz, 1H), 4.84 (s, 1H), 4.40 (t, J=7.1 Hz, 2H), 3.68 (d, J=10.1 Hz, 2H), 3.38 (d, J=27.3 Hz, 2H), 2.83 (s, 1H), 2.31 (d, J 7.6 Hz, 2H), 2.09 (t, J=7.2 Hz, 2H), 2.01 (dd, J=11.9, 7.0 Hz, 1H), 1.92 (s, 1H), 1.72 (t, J=11.3 Hz, 1H), 1.60 (s, 3H), 1.56 (s, 3H), 1.05-0.99 (m, 2H), 0.74 (q, J=3.0, 1.7 Hz, 2H). ESI-MS m/z calc. 709.2658, found 710.2 (M+1)⁺; Retention time: 1.38 minutes (LC method M).

### Example 129: Preparation of 3-[(14S)-12,12-dimethyl-2,2,4-trioxo-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaen-18-yl]propenamide (Compound 378)

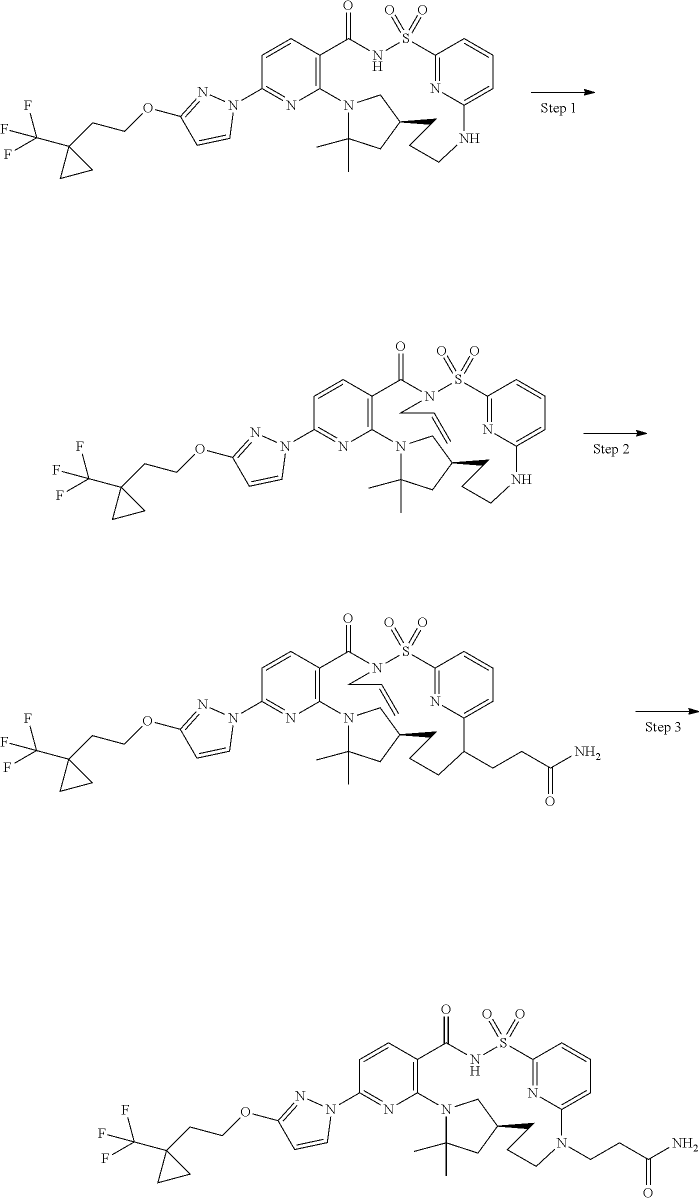 [SMILES: CC1(C)C[C@@H]2CCCNc3cccc(n3)S(=O)(=O)NC(=O)c3ccc(-n4ccc(OCCC5(C(F)(F)F)CC5)n4)nc3N1C2 ; C=CCN1C(=O)c2ccc(-n3ccc(OCCC4(C(F)(F)F)CC4)n3)nc2N2C[C@@H](CCCNc3cccc(n3)S1(=O)=O)CC2(C)C ; C=CCN1C(=O)c2ccc(-n3ccc(OCCC4(C(F)(F)F)CC4)n3)nc2N2C[C@@H](CCCC(CCC(N)=O)c3cccc(n3)S1(=O)=O)CC2(C)C ; CC1(C)C[C@@H]2CCCN(CCC(N)=O)c3cccc(n3)S(=O)(=O)NC(=O)c3ccc(-n4ccc(OCCC5(C(F)(F)F)CC5)n4)nc3N1C2]

### Step 1: (14S)-12,12-Dimethyl-3-(prop-2-en-1-yl)-8-(3-{2-[1-(trifluoromethyl)cyclo propyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione

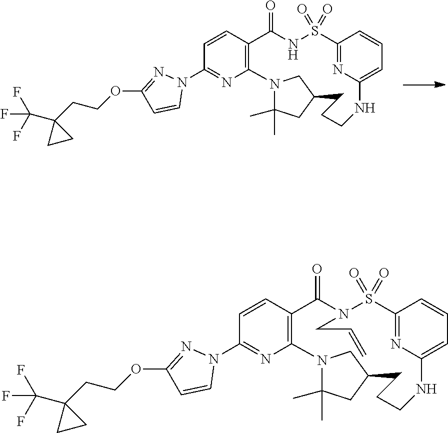 [SMILES: CC1(C)C[C@@H]2CCCNc3cccc(n3)S(=O)(=O)NC(=O)c3ccc(-n4ccc(OCCC5(C(F)(F)F)CC5)n4)nc3N1C2 ; C=CCN1C(=O)c2ccc(-n3ccc(OCCC4(C(F)(F)F)CC4)n3)nc2N2C[C@@H](CCCNc3cccc(n3)S1(=O)=O)CC2(C)C]

To a 20 mL vial was added (14S)-12,12-dimethyl-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(23),5,7,9,19,21-hexaene-2,2,4-trione (500 mg, 0.79 mmol), potassium carbonate (437 mg, 3.16 mmol), and dry DMSO (5 mL). The reaction was allowed to stir at rt and allyl bromide (76 μL, 0.8783 mmol) was added. The reaction was capped and allowed to stir at rt overnight. An additional amount (40 μL, 0.4622 mmol) of allyl bromide was added and the reaction was allowed to stir for an additional 3 hours. The reaction was filtered and purified via reverse phase column chromatography 50%-99% ACN:H₂O to provide (14S)-12,12-dimethyl-3-(prop-2-en-1-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (480 mg, 90%) as a white solid. ESI-MS m/z calc. 673.2658, found 674.4 (M+1)⁺; Retention time: 2.1 minutes (LC method G).

### Step 2: 3-[(14S)-12,12-Dimethyl-2,2,4-trioxo-3-(prop-2-en-1-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaen-18-yl]propenamide

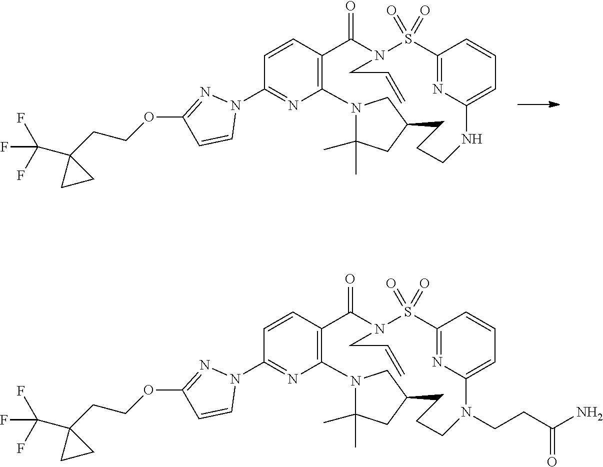 [SMILES: C=CCN1C(=O)c2ccc(-n3ccc(OCCC4(C(F)(F)F)CC4)n3)nc2N2C[C@@H](CCCNc3cccc(n3)S1(=O)=O)CC2(C)C ; C=CCN1C(=O)c2ccc(-n3ccc(OCCC4(C(F)(F)F)CC4)n3)nc2N2C[C@@H](CCCN(CCC(N)=O)c3cccc(n3)S1(=O)=O)CC2(C)C]

To a microwave vial was added (14S)-12,12-dimethyl-3-(prop-2-en-1-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (30 mg, 0.045 mmol), DMF (0.5 mL), and sodium hydride (10.7 mg of 60% w/w, 0.27 mmol). After stirring for 5 minutes at rt, the reaction turned bright yellow. 3-Bromopropanamide (10.2 mg, 0.067 mmol) was added. After 3 hours, additional sodium hydride (10.7 mg of 60% w/w, 0.2675 mmol) followed by 3-bromopropanamide (10.2 mg, 0.06711 mmol) was added and the reaction was allowed to stir for 3 days. The reaction was filtered and purified by HPLC (30%-99%) ACN:H₂O with an HCl modifier. 3-[(145)-12,12-dimethyl-2,2,4-trioxo-3-(prop-2-en-1-yl)-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(23),5(10),6,8,19,21-hexaen-18-yl]propanamide was isolated as a yellow solid (8 mg, 24%). ESI-MS m/z calc. 744.3029, found 745.5 (M+1)⁺; Retention time: 1.84 minutes (LC method G).

### Step 3: 3-[(14S)-12,12-Dimethyl-2,2,4-trioxo-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaen-18-yl]propanamide (Compound 378)

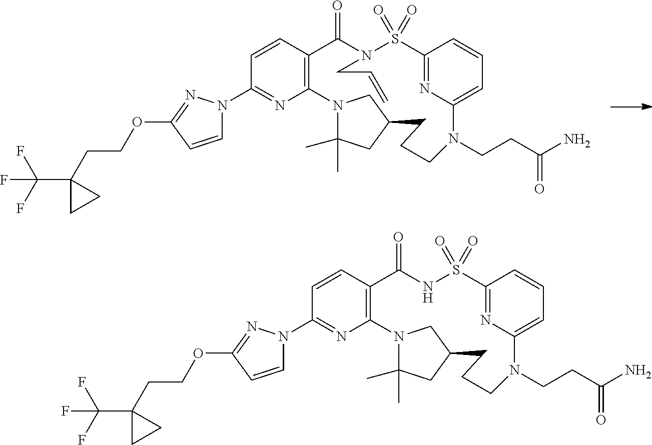 [SMILES: C=CCN1C(=O)c2ccc(-n3ccc(OCCC4(C(F)(F)F)CC4)n3)nc2N2C[C@@H](CCCN(CCC(N)=O)c3cccc(n3)S1(=O)=O)CC2(C)C ; CC1(C)C[C@@H]2CCCN(CCC(N)=O)c3cccc(n3)S(=O)(=O)NC(=O)c3ccc(-n4ccc(OCCC5(C(F)(F)F)CC5)n4)nc3N1C2]

To a test tube containing 3-[(145)-12,12-dimethyl-2,2,4-trioxo-3-(prop-2-en-1-yl)-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaen-18-yl]propanamide (8 mg, 0.012 mmol) was added 1,2-dichloroethane (0.5 mL). Pd(PPh₃)₄ (12.5 mg, 0.012 mmol) was added and the reaction was stirred for 5 minutes until the reaction turned dark red. Benzenesulfonate (Sodium salt) (2 mg, 0.087 mmol) was added and the reaction was stirred at rt for 10 minutes. The reaction was filtered and purified via HPLC (30%-99%) ACN:H₂O with an HCl modifier. 3-[(14S)-12,12-dimethyl-2,2,4-trioxo-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaen-18-yl]propanamide was isolated as a solid (7.4 mg, 98%). ESI-MS m/z calc. 704.2716, found 705.4 (M+1)⁺; Retention time: 1.51 minutes (LC method G).

The following is a list of alkyl halide reagents that are commercially available:
3-(2-Chloroethoxy)propanenitrile
2-Chloro-N,N-dimethyl-acetamide
3-(Bromomethyl)-4-methyl-1,2,4-triazole
6-Bromohexanenitrile
3-(Chloromethyl)oxetane

The compounds in the following table were prepared in a manner analogous to that described above using alkyl halides given in the table above:

| Com- | | LCMS | | | |
|---|---|---|---|---|---|
| pound | | Retention | | | |
| Num- | | Time | Exact | | LCMS |
| ber | Structure | (min) | Mass | M + 1 | Method |
| 359 | 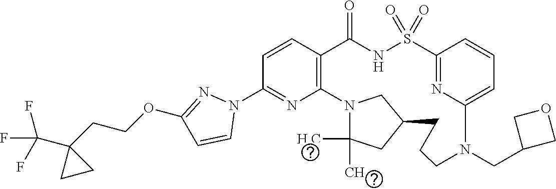 | 1.77 | 703.276 | 704.4 | LC Method G |
| 360 | 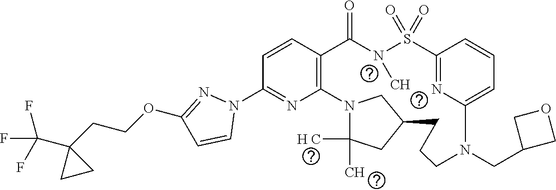 | 2.09 | 743.308 | 744.5 | LC Method G |
| 369 | 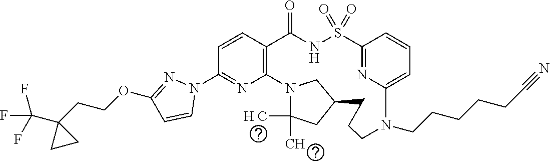 | 1.93 | 728.308 | 729.4 | LC Method G |
| 373 | 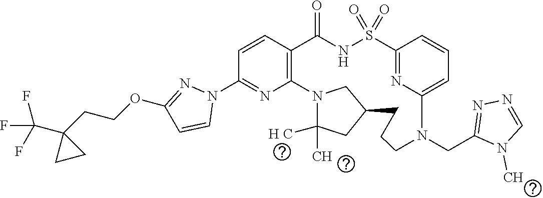 | 1.33 | 728.283 | 729.3 | LC Method G |
| 376 | 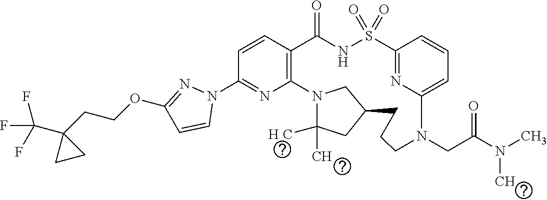 | 1.68 | 718.287 | 719.3 | LC Method G |
| 377 | 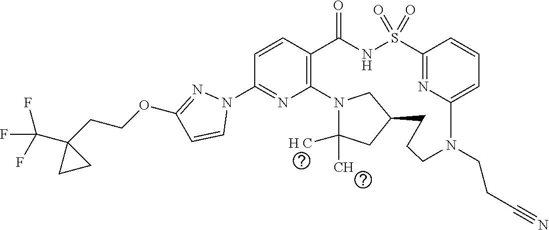 | 1.85 | 686.261 | 687.3 | LC Method G |

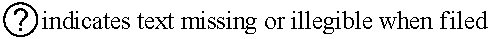

### Example 130 Preparation of (14S)-18-[(2S)-2,3-dihydroxypropyl]-12,12-dimethyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (Compound 381) and (14S)-18-[(2R)-2,3-dihydroxypropyl]-12,12-dimethyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (Compound 379)

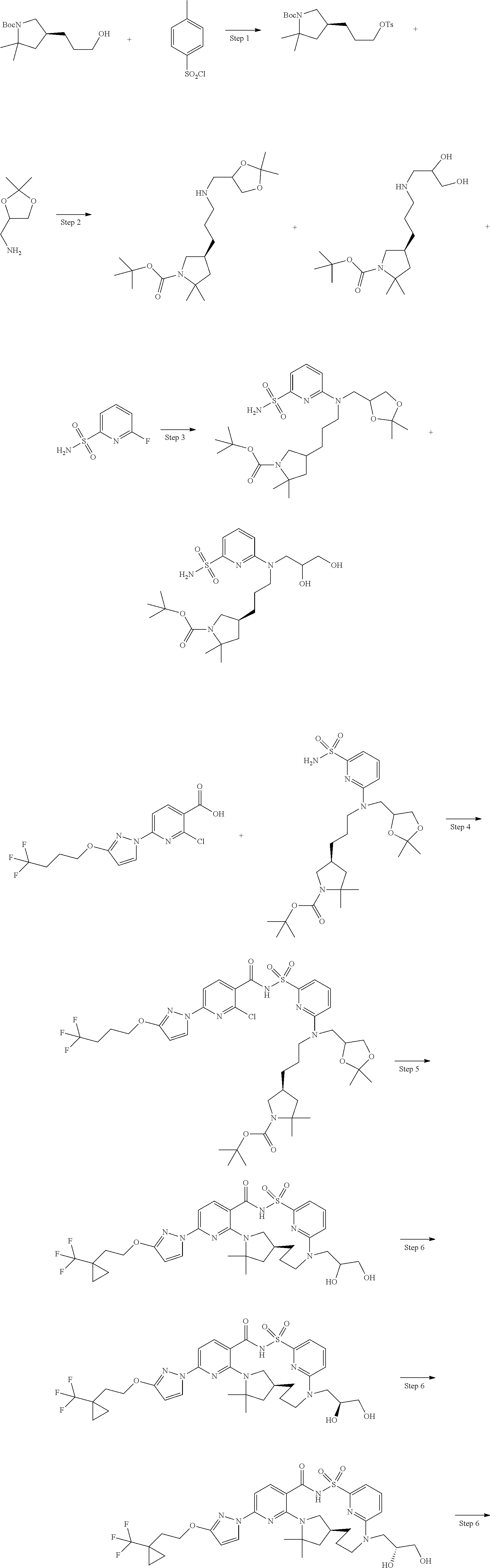

### Step 1: tert-Butyl (4S)-2,2-dimethyl-4-[3-(p-tolylsulfonyloxy)propyl]pyrrolidine-1-carboxylate

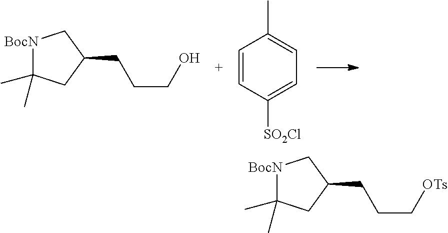

A 500 mL 3 neck round-bottom flask was fitted with a mechanical stirrer, a cooling bath, an addition funnel, a J-Kem temperature probe and a nitrogen inlet/outlet. The vessel was charged under a nitrogen atmosphere with tert-butyl (4S)-4-(3-hydroxypropyl)-2,2-dimethyl-pyrrolidine-1-carboxylate (10 g, 38.85 mmol) and dichloromethane (250 mL) which provided a clear colorless solution. Stirring was commenced and the pot temperature was recorded at 19° C. The cooling bath was charged with crushed ice/water and the pot temperature was lowered to 0° C. The addition funnel was charged with triethylamine (5.11 g, 50.50 mmol) which was added neat dropwise over 5 minutes. The addition funnel was then charged with a solution of p-toluenesulfonyl chloride (8.148 g, 42.74 mmol) in dichloromethane (15 mL) which was added dropwise over 20 minutes which resulted in a clear light yellow solution and an exotherm to 2° C. The mixture was continued to stir at <5° C. for 1 hour and then allowed to warm to rt and stir for 8 hours. The reaction mixture was further diluted with dichloromethane (200 mL) and then poured into a saturated ammonium chloride solution (250 mL). The biphasic mixture was then transferred to a separatory funnel. The organic was removed, washed with a saturated ammonium chloride solution (150 mL), dried over sodium sulfate (150 g) and then filtered through a glass frit Buchner funnel. The filtrate was concentrated under reduced pressure to provide tert-butyl (4S)-2,2-dimethyl-4-[3-(p-tolylsulfonyloxy)propyl]pyrrolidine-1-carboxylate (14.23 g, 89% yield) of a clear amber oil as the desired product.

### Step 2: tert-Butyl (4S)-4-[3-[(2,2-dimethyl-1,3-dioxolan-4-yl)methylamino]propyl]-2,2-dimethyl-pyrrolidine-1-carboxylate and tert-butyl (4S)-4-[3-(2,3-dihydroxypropylamino)propyl]-2,2-dimethyl-pyrrolidine-1-carboxylate

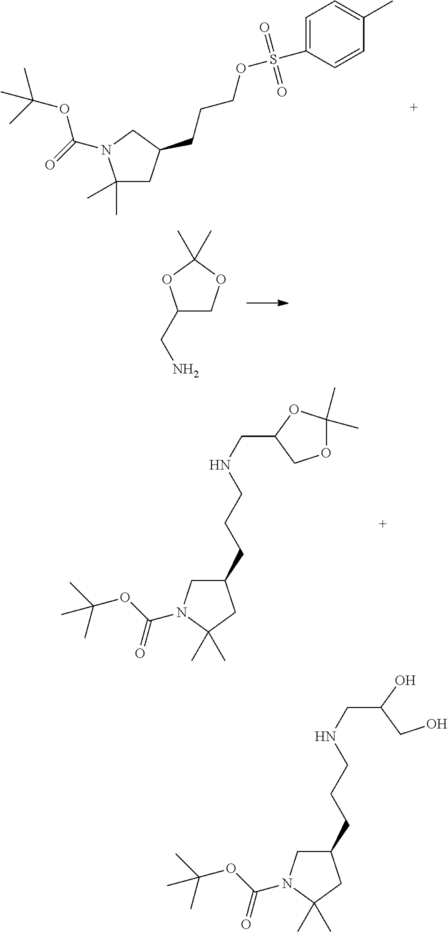

To a solution of tert-butyl (4S)-2,2-dimethyl-4-[3-(p-tolylsulfonyloxy)propyl]pyrrolidine-1-carboxylate (200 mg, 0.4860 mmol) in acetonitrile (2 mL) was added (2,2-dimethyl-1,3-dioxolan-4-yl)methanamine (160 μL, 1.234 mmol) followed by DIEA (450 μL, 2.584 mmol) and the reaction mixture was stirred at rt for overnight. The mixture was heated at 50° C. for 6h. The reaction mixture was filtered through Whatman filter disc (puradisc 25 TF) and the filtrate was purified by a reverse phase HPLC-MS method using a dual gradient run from 1-99% mobile phase B over 15 minutes (mobile phase A=H₂0 (no modifier), mobile phase B=CH₃CN) to afford after evaporation a mixture of tert-butyl (4S)-4-[3-[(2,2-dimethyl-1,3-dioxolan-4-yl)methylamino]propyl]-2,2-dimethyl-pyrrolidine-1-carboxylate and 35% of tert-butyl (4S)-4-[3-(2,3-dihydroxypropylamino)propyl]-2,2-dimethyl-pyrrolidine-1-carboxylate (120 mg, 67%). The mixture was used for the next step. ESI-MS m/z calc. 370.28317, found 371.44 (M+1)⁺; Retention time: 0.54 minutes, side product ESI-MS m/z calc. 330.25186, found 331.4 (M+1)⁺; Retention time: 0.45 minutes (LC method D).

### Step 3: tert-Butyl (4S)-4-[3-[(2,2-dimethyl-1,3-dioxolan-4-yl)methyl-(6-sulfamoyl-2-pyridyl)amino]propyl]-2,2-dimethyl-pyrrolidine-1-carboxylate and tert-butyl (4S)-4-[3-[2,3-dihydroxypropyl-(6-sulfamoyl-2-pyridyl)amino]propyl]-2,2-dimethyl-pyrrolidine-1-carboxylate

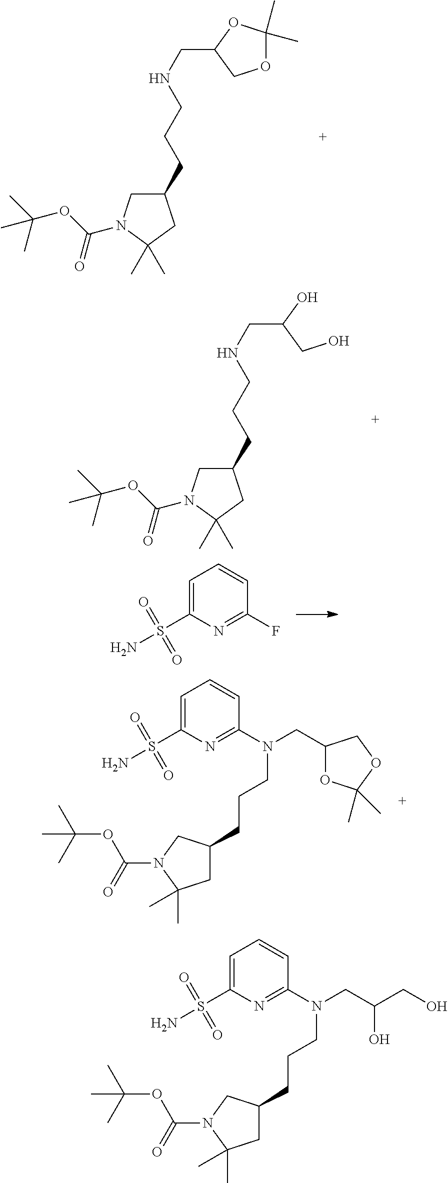

To a mixture of tert-butyl (4S)-4-[3-[(2,2-dimethyl-1,3-dioxolan-4-yl)methylamino]propyl]-2,2-dimethyl-pyrrolidine-1-carboxylate (120 mg, 0.32 mmol) containing 35% of tert-butyl (4S)-4-[3-(2,3-dihydroxypropylamino)propyl]-2,2-dimethyl-pyrrolidine-1-carboxylate, 6-fluoropyridine-2-sulfonamide (62 mg, 0.35 mmol) and DMSO (2 mL) was added DIEA (300 μL, 1.722 mmol). The mixture was stirred at 80° C. for 18 h, then at 120° C. for 2 days. The reaction mixture was cooled to ambient temperature and filtered through Whatman filter disc (puradisc 25 TF) and the filtrate was purified by a reverse phase HPLC-MS method using a dual gradient run from 1-99% mobile phase B over 15 minutes (mobile phase A=H₂O (no modifier), mobile phase B=CH₃CN) to afford separately, tert-butyl (4S)-4-[3-[(2,2-dimethyl-1,3-dioxolan-4-yl)methyl-(6-sulfamoyl-2-pyridyl)amino]propyl]-2,2-dimethyl-pyrrolidine-1-carboxylate (65 mg, 38%) ESI-MS m/z calc. 526.28253, found 527.13 (M+1)⁺; Retention time: 0.71 minutes (LC method D) and tert-butyl (4S)-4-[3-[2,3-dihydroxypropyl-(6-sulfamoyl-2-pyridyl)amino]propyl]-2,2-dimethyl-pyrrolidine-1-carboxylate (23 mg, 15%), ESI-MS m/z calc. 486.25122, found 487.13 (M+1)⁺; Retention time: 0.58 minutes (LC method D).

### Step 4: tert-Butyl (4S)-4-[3-[[6-[[2-chloro-6-[3-[2-[1-(trifluoromethyl)cyclopropyl] ethoxy]pyrazol-1-yl]pyridine-3-carbonyl]sulfamoyl]-2-pyridyl]-[(2,2-dimethyl-1,3-dioxolan-4-yl)methyl]amino]propyl]-2,2-dimethyl-pyrrolidine-1-carboxylate

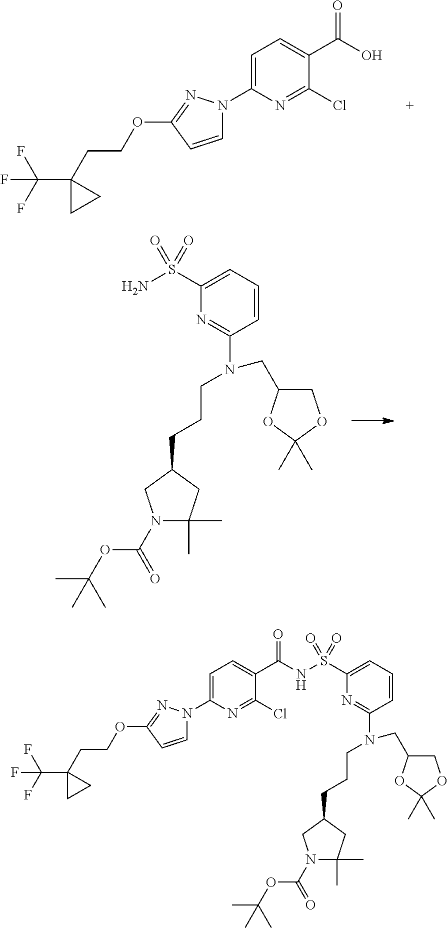 [SMILES: O=C(O)c1ccc(-n2ccc(OCCC3(C(F)(F)F)CC3)n2)nc1Cl ; CC(C)(C)OC(=O)N1C[C@@H](CCCN(CC2COC(C)(C)O2)c2cccc(S(N)(=O)=O)n2)CC1(C)C ; CC(C)(C)OC(=O)N1C[C@@H](CCCN(CC2COC(C)(C)O2)c2cccc(S(=O)(=O)NC(=O)c3ccc(-n4ccc(OCCC5(C(F)(F)F)CC5)n4)nc3Cl)n2)CC1(C)C]

To a solution of 2-chloro-6-[3-[2-[1-(trifluoromethyl)cyclopropyl]ethoxy] pyrazol-1-yl]pyridine-3-carboxylic acid (47 mg, 0.12 mmol) in THF (2 mL) was added CDI (23 mg, 0.14 mmol) (recrystallized from THF) and the mixture was stirred at rt for 3 h, then was added as a solution to tert-butyl (4S)-4-[3-[(2,2-dimethyl-1,3-dioxolan-4-yl)methyl-(6-sulfamoyl-2-pyridyl)amino]propyl]-2,2-dimethyl-pyrrolidine-1-carboxylate (60 mg, 0.11 mmol) in THF (1 mL) followed by DBU (40 μL, 0.27 mmol) and the resulting mixture was stirred for 18 h at rt. The reaction mixture was filtered through Whatman filter disc (puradisc 25 TF) and the filtrate was purified by a reverse phase HPLC-MS method using a dual gradient run from 50-99% mobile phase B over 15.0 minutes (mobile phase A=H₂0 containing 10 mM ammonium formate; mobile phase B=CH₃CN). The product containing fractions were combined and lyophilized to afford tert-butyl (4S)-4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]-[(2,2-dimethyl-1,3-dioxolan-4-yl)methyl]amino]propyl]-2,2-dimethyl-pyrrolidine-1-carboxylate (68 mg, 68%) as a white solid. ESI-MS m/z calc. 883.33167, found 884.36 (M+1)⁺; Retention time: 0.62 minutes (LC method U).

### Step 5: (14S)-18-(2,3-dihydroxypropyl)-12,12-dimethyl-8-(3-{2-[1-(trifluoromethyl) cyclopropyl]ethoxy}-1H-pyrazol-1-yl)-2λ⁶-thia-3,9,11,18,23-pentaazatetracyclo [17.3.1.111,14.05,10]tetracosa-1(22),5,7,9,19(23),20-hexaene-2,2,4-trione (Compound 382)

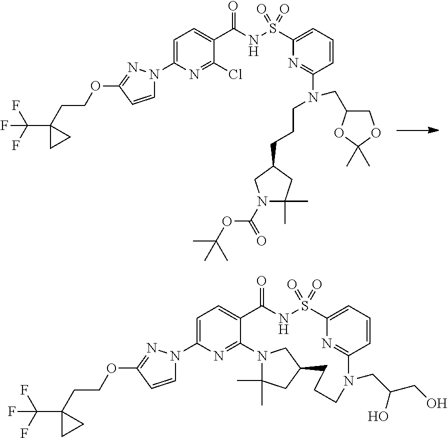 [SMILES: CC(C)(C)OC(=O)N1C[C@@H](CCCN(CC2COC(C)(C)O2)c2cccc(S(=O)(=O)NC(=O)c3ccc(-n4ccc(OCCC5(C(F)(F)F)CC5)n4)nc3Cl)n2)CC1(C)C ; CC1(C)C[C@@H]2CCCN(CC(O)CO)c3cccc(n3)S(=O)(=O)NC(=O)c3ccc(-n4ccc(OCCC5(C(F)(F)F)CC5)n4)nc3N1C2]

A solution of tert-butyl (4S)-4-[3-[[6-[[2-chloro-6-[3-[2-[1-(trifluoromethyl) cyclopropyl]ethoxy]pyrazol-1-yl]pyridine-3-carbonyl]sulfamoyl]-2-pyridyl]-[(2,2-dimethyl-1,3-dioxolan-4-yl)methyl]amino]propyl]-2,2-dimethyl-pyrrolidine-1-carboxylate (68 mg, 0.077 mmol) in TFA (30 μL, 0.389 mmol) and DCM (150 μL) was stirred at rt for 2 h. After completion of the reaction, solvents were removed. The residue was dissolved in ethyl acetate and washed with a saturated NaHCO₃ solution (2 mL) and the organic layer was collected and the solvent removed. The material was dissolved in dimethyl sulfoxide (2 mL). Oven dried 4 Å molecular sieves were added and the mixture was stirred for 10 min. Then, potassium carbonate (42 mg, 0.304 mmol) was added and the reaction mixture was heated at 140° C. for 16 h. The reaction mixture was filtered through a Whatman filter disc (puradisc 25 TF) and the filtrate was purified by a reverse phase HPLC-MS method using a dual gradient run from 50-99% mobile phase B over 15 minutes (mobile phase A=H₂0 containing 5 mM HCl; mobile phase B=CH₃CN) to afford (14S)-18-(2,3-dihydroxypropyl)-12,12-dimethyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (32 mg, 59%) as an off white solid. ESI-MS m/z calc. 707.2713, found 708.22 (M+1)⁺; Retention time: 1.54 minutes (LC method G).

### Step 6: (14S)-18-[(2S)-2,3-dihydroxypropyl]-12,12-dimethyl-8-(3-{2-[1-(trifluoromethyl) cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (Compound 381) and (14S)-18-[(2R)-2,3-dihydroxypropyl]-12,12-dimethyl-8-(3-{2-[1-(trifluoromethyl) cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (Compound 379)

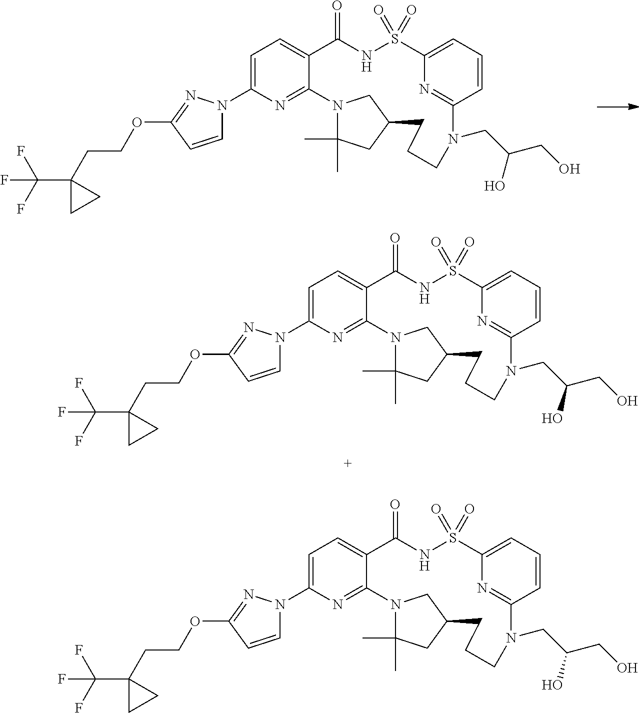 [SMILES: CC1(C)C[C@@H]2CCCN(CC(O)CO)c3cccc(n3)S(=O)(=O)NC(=O)c3ccc(-n4ccc(OCCC5(C(F)(F)F)CC5)n4)nc3N1C2 ; CC1(C)C[C@@H]2CCCN(C[C@H](O)CO)c3cccc(n3)S(=O)(=O)NC(=O)c3ccc(-n4ccc(OCCC5(C(F)(F)F)CC5)n4)nc3N1C2 ; CC1(C)C[C@@H]2CCCN(C[C@@H](O)CO)c3cccc(n3)S(=O)(=O)NC(=O)c3ccc(-n4ccc(OCCC5(C(F)(F)F)CC5)n4)nc3N1C2]

(14S)-18-(2,3-Dihydroxypropyl)-12,12-dimethyl-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (25 mg, 0.0353 mmol) (diastereomeric mixture) was subjected to chiral separation by SFC chromatography using a ChiralPak IC (250×10 mm), 5 nm particle size column, using 30% MeOH-62% CO₂ at 10 mL/min over 16 min, giving two isomers as white solids:

The first isomer to elute (PEAK-1) was (14S)-18-[(2S)-2,3-dihydroxypropyl]-12,12-dimethyl-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (9.4 mg, 75%). ESI-MS m/z calc. 707.2713, found 708.11 (M+1)⁺; Retention time: 1.54 minutes (LC method G).

The second enantiomer to elute (PEAK-2) was (145)-18-[(2R)-2,3-dihydroxypropyl]-12,12-dimethyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (5.2 mg, 42%). ¹H NMR (500 MHz, DMSO-d₆) δ 12.61 (s, 1H), 8.22 (d, J=2.7 Hz, 1H), 7.78 (d, J=8.2 Hz, 1H), 7.72 (t, J=8.0 Hz, 1H), 7.18 (d, J=7.3 Hz, 1H), 7.08 (s, 1H), 6.92 (d, J=8.2 Hz, 1H), 6.12 (d, J=2.7 Hz, 1H), 4.95 (d, J=5.2 Hz, 1H), 4.81 (s, 1H), 4.32 (t, J=7.3 Hz, 3H), 3.70 (s, 1H), 3.59 (d, J=14.8 Hz, 1H), 3.40 (d, J=11.1 Hz, 2H), 3.22 (dd, J=15.3, 8.5 Hz, 1H), 2.18 (s, 1H), 2.08 (t, J=7.1 Hz, 3H), 1.86 (s, 1H), 1.61 (s, 3H), 1.52 (s, 6H), 1.34 (d, J=19.9 Hz, 1H), 1.23 (d, J=10.0 Hz, 2H), 1.11 (s, 1H), 0.96 (d, J=5.0 Hz, 2H), 0.89 (s, 2H). ESI-MS m/z calc. 707.2713, found 708.22 (M+1)⁺; Retention time: 1.51 minutes (LC method G).

### Example 131: Preparation of (14S)-12,12-dimethyl-18-phenyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (Compound 395)

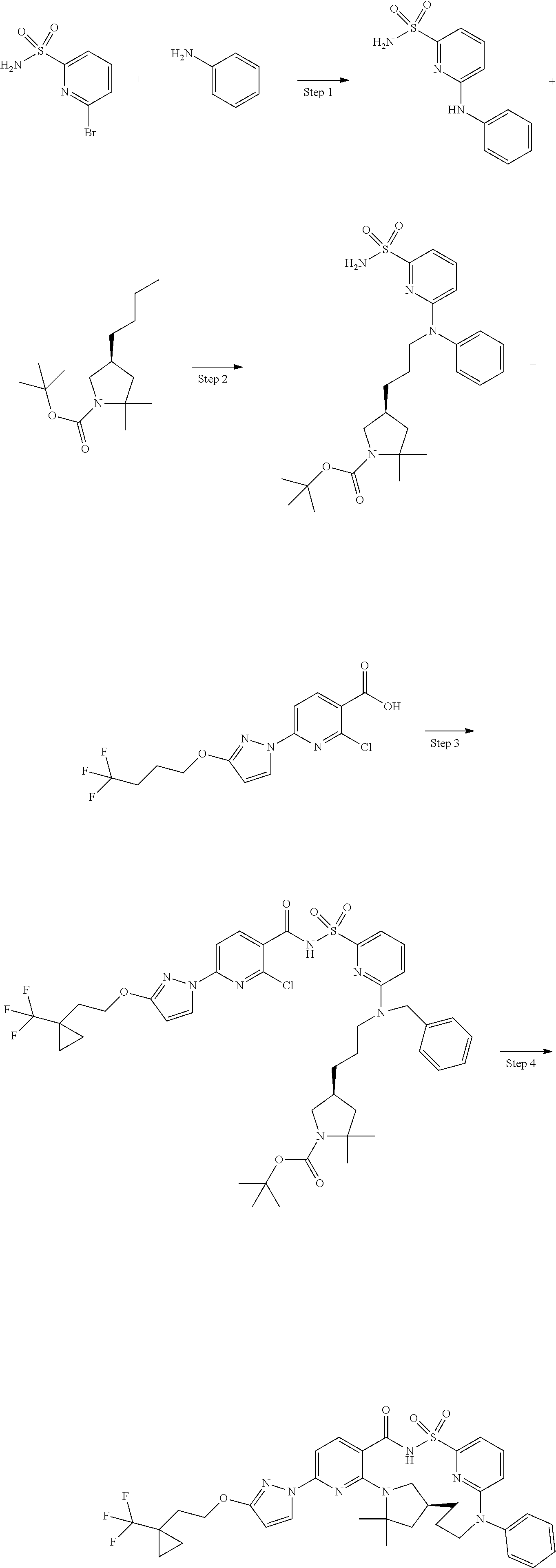

### Step 1: 6-Anilinopyridine-2-sulfonamide

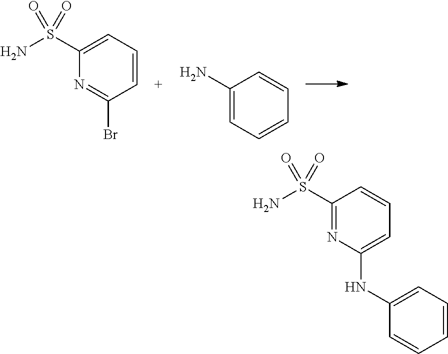

To 6-bromopyridine-2-sulfonamide (1.0 g, 4.218 mmol), Pd₂(dba)₃ (408 mg, 0.446 mmol), Xantphos (490 mg, 0.847 mmol) and cesium carbonate (4.25 g, 13.04 mmol) under nitrogen was added degassed 1,4-dioxane (22 mL) followed by aniline (475 μL, 5.213 mmol). The mixture was heated in an oil bath at 120° C. for 20 h. The mixture was cooled to ambient temperature and saturated ammonium chloride was added and the mixture was extracted with ethyl acetate. The combined organic extracts were washed with brine, dried over sodium sulfate, filtered and concentrated. The resultant brown residue was purified by silica gel column chromatography (40 gram column) using a shallow gradient of 100% hexanes to 100% ethyl acetate to afford 6-anilinopyridine-2-sulfonamide (370 mg, 35% as a light yellow solid. ¹H NMR (400 MHz, DMSO-d₆) δ 9.44 (s, 1H), 7.91-7.62 (m, 3H), 7.44-7.19 (m, 5H), 7.06-6.80 (m, 2H). ESI-MS m/z calc. 249.05719, found 250.2 (M+1)⁺; Retention time: 0.9 minutes (LC method A).

### Step 2: tert-Butyl (4S)-2,2-dimethyl-4-[3-(N-(6-sulfamoyl-2-pyridyl)anilino)propyl] pyrrolidine-1-carboxylate

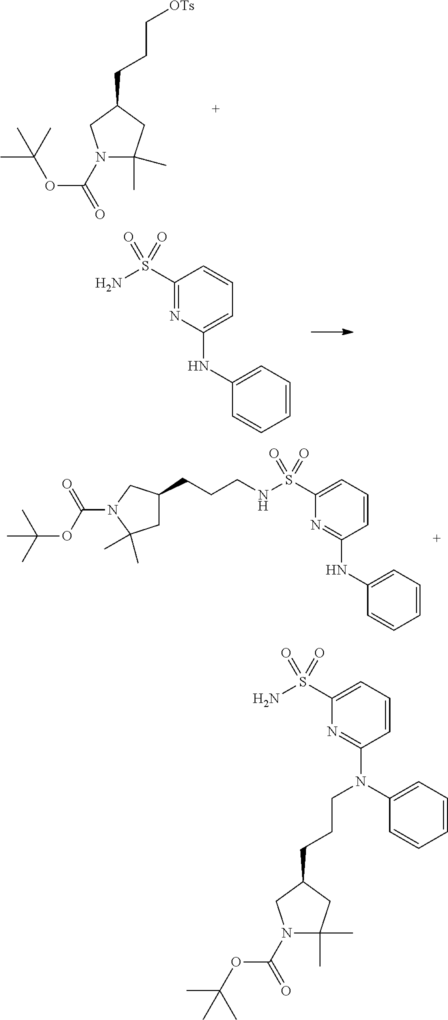

6-Anilinopyridine-2-sulfonamide (445 mg, 1.785 mmol) and NaH (160 mg of 60% w/w, 4.00 mmol) were combined in DMSO (11 mL) and stirred for 15 minutes. Then tert-butyl(4S)-2,2-dimethyl-4-[3-(p-tolylsulfonyloxy)propyl]pyrrolidine-1-carboxylate (685 mg, 1.664 mmol) was added and the mixture was allowed to stir for 90 min. This crude mixture was filtered, diluted with ethyl acetate and washed with water (2×20 mL), followed by brine. The organics were further washed with water, then separated, dried over sodium sulfate, and evaporated. The residue was purified on silica gel chromatography (80 gram column) using a gradient from 100% hexanes to 60% ethyl acetate in hexanes to isolate two products:

More polar off-white solid tert-butyl (4S)-4-[3-[(6-anilino-2-pyridyl)sulfonyl amino]propyl]-2,2-dimethyl-pyrrolidine-1-carboxylate (65 mg, 8%) ESI-MS m/z calc. 488.24573, found 489.2 (M+1)⁺; Retention time: 1.91 minutes (LC method A).

Less polar white solid tert-butyl (4S)-2,2-dimethyl-4-[3-(N-(6-sulfamoyl-2-pyridypanilino)propyl]pyrrolidine-1-carboxylate (347 mg, 43%). ¹H NMR (400 MHz, DMSO-d₆) δ 7.58-7.45 (m, 3H), 7.40-7.28 (m, 3H), 7.23 (d, J=3.0 Hz, 2H), 7.11 (d, J=7.1 Hz, 1H), 6.41-6.32 (m, 1H), 4.00 (dt, J=16.9, 9.5 Hz, 2H), 3.47 (q, J=10.6 Hz, 1H), 2.76-2.67 (m, 1H), 2.06 (d, J=18.4 Hz, 1H), 1.87-1.78 (m, 1H), 1.53 (q, J=7.5 Hz, 2H), 1.35 (dd, J=19.8, 11.1 Hz, 15H), 1.20 (s, 3H). ESI-MS m/z calc. 488.24573, found 489.2 (M+1)⁺; Retention time: 2.0 minutes (LC method A).

### Step 3: tert-Butyl (4S)-4-[3-(N-[6-[[2-chloro-6-[3-[2-[1-(trifluoromethyl)cyclopropyl]ethoxy]pyrazol-1-yl]pyridine-3-carbonyl]sulfamoyl]-2-pyridyl]anilino)propyl]-2,2-dimethyl-pyrrolidine-1-carboxylate

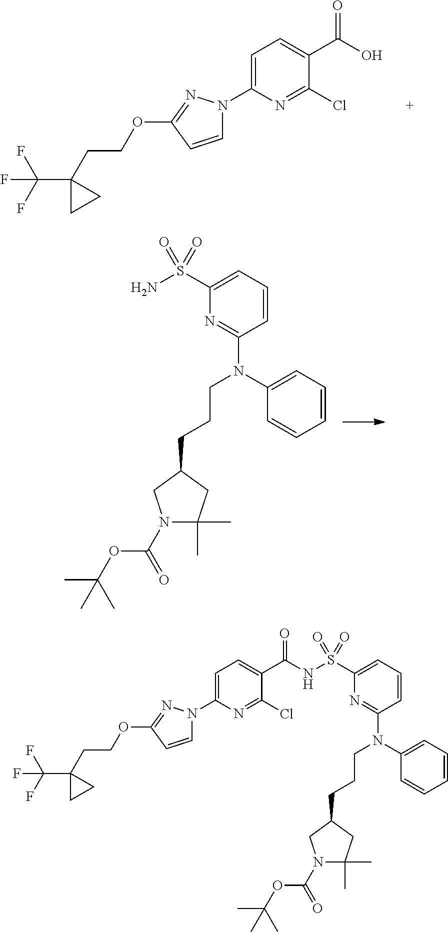 [SMILES: O=C(O)c1ccc(-n2ccc(OCCC3(C(F)(F)F)CC3)n2)nc1Cl ; CC(C)(C)OC(=O)N1C[C@@H](CCCN(c2ccccc2)c2cccc(S(N)(=O)=O)n2)CC1(C)C ; CC(C)(C)OC(=O)N1C[C@@H](CCCN(c2ccccc2)c2cccc(S(=O)(=O)NC(=O)c3ccc(-n4ccc(OCCC5(C(F)(F)F)CC5)n4)nc3Cl)n2)CC1(C)C]

To a solution of 2-chloro-6-[3-[2-[1-(trifluoromethyl)cyclopropyl]ethoxy] pyrazol-1-yl]pyridine-3-carboxylic acid (250 mg, 0.665 mmol) in THF (5.0 mL) was added CDI (130 mg, 0.802 mmol) (recrystallized from THF) and the mixture was stirred at rt for 3 h then tert-butyl (4S)-2,2-dimethyl-4-[3-(N-(6-sulfamoyl-2-pyridyl)anilino)propyl] pyrrolidine-1-carboxylate (340 mg, 0.696 mmol) was added as a solution in THF (1.25 mL) followed by DBU (310 μL, 2.073 mmol) and the resulting mixture was stirred for 18 h at rt. The reaction was diluted with ethyl acetate and washed with water (2×20 mL), followed by brine. The organics were further washed with water, then separated, dried over sodium sulfate, and evaporated. The residue was purified by silica gel chromatography (80 gram column) using a gradient from 100% hexanes to 50% ethyl acetate in hexanes to afford as an off-white solid tert-butyl (4S)-4-[3-(N-[6-[[2-chloro-6-[3-[2-[1-(trifluoromethyl)cyclopropyl]ethoxy]pyrazol-1-yl]pyridine-3-carbonyl]sulfamoyl]-2-pyridyl]anilino)propyl]-2,2-dimethyl-pyrrolidine-1-carboxylate (135 mg, 24%). ESI-MS m/z calc. 845.2949, found 846.6 (M+1)⁺; Retention time: 2.3 minutes (LC method A).

### Step 4: (14S)-12,12-Dimethyl-18-phenyl-8-(3-{2-[1-(trifluoromethyl)cyclopropyl] ethoxy}-1H-pyrazol-1-yl)-2λ⁶-thia-3,9,11,18,23-pentaazatetracyclo [17.3.1.111,14.05,10]tetracosa-1(23),5(10),6,8,19,21-hexaene-2,2,4-trione (Compound 395)

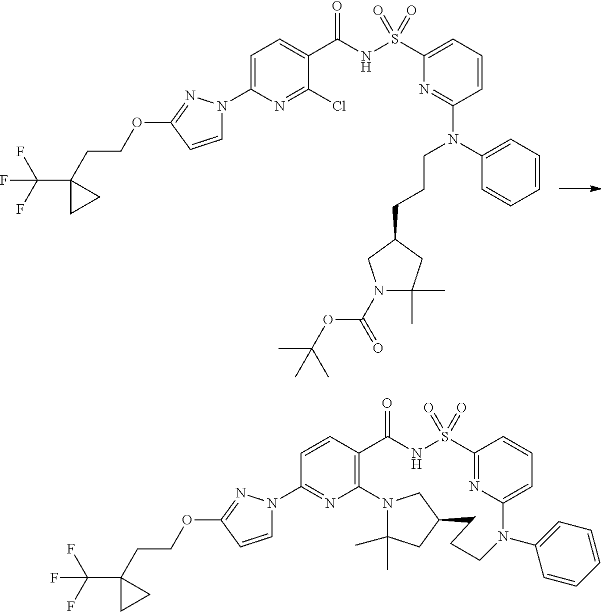 [SMILES: CC(C)(C)OC(=O)N1C[C@@H](CCCN(c2ccccc2)c2cccc(S(=O)(=O)NC(=O)c3ccc(-n4ccc(OCCC5(C(F)(F)F)CC5)n4)nc3Cl)n2)CC1(C)C ; CC1(C)C[C@@H]2CCCN(c3ccccc3)c3cccc(n3)S(=O)(=O)NC(=O)c3ccc(-n4ccc(OCCC5(C(F)(F)F)CC5)n4)nc3N1C2]

tert-Butyl (4S)-4-[3-(N-[6-[[2-chloro-6-[3-[2-[1-(trifluoromethyl)cyclopropyl] ethoxy]pyrazol-1-yl]pyridine-3-carbonyl]sulfamoyl]-2-pyridyl]anilino)propyl]-2,2-dimethyl-pyrrolidine-1-carboxylate (135 mg, 0.159 mmol) was dissolved in DCM (5 mL) and to the mixture was added HCl (4M in dioxane) (1000 μL, 4.00 mmol) and the resulting mixture was stirred at room temperature for 90 minutes. The mixture was evaporated to dryness, then diluted with diethyl ether (5 mL×2), concentrated, and placed on the high vacuum pump overnight affording the crude intermediate 2-chloro-N-[[6-[N-[3-[(3S)-5,5-dimethylpyrrolidin-3-yl]propyl]anilino]-2-pyridyl]sulfonyl]-6-[3-[2-[1-(trifluoromethyl)cyclopropyl]ethoxy]pyrazol-1-yl]pyridine-3-carboxamide (hydrochloride salt) as a viscous oil (120 mg, 96%). ESI-MS m/z calc. 745.2425, found 746.5 (M+1)⁺; Retention time: 1.12 minutes. The material was combined with K₂CO₃ (160 mg, 1.158 mmol), 3 Å molecular sieves and DMSO (8 mL) in a vial, which was purged with nitrogen, capped, heated to 155° C. and stirred for 16 h. The mixture was filtered, diluted with ethyl acetate and washed with water (2×20 mL), followed by brine. The organics were further washed with water, then separated, dried over sodium sulfate, and evaporated. The residue was purified on silica gel chromatography (12 gram column) using a gradient from 100% hexanes to 70% ethyl acetate in hexanes to afford as an off-white solid (14S)-12,12-dimethyl-18-phenyl-8-(3-{2-[1-(trifluoromethyl) cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (52.32 mg, 46%). ¹H NMR (400 MHz, DMSO-d₆) δ 12.70 (s, 1H), 8.23 (d, J=2.8 Hz, 1H), 7.86 (d, J=8.2 Hz, 1H), 7.68-7.53 (m, 3H), 7.48-7.35 (m, 3H), 7.26 (d, J=7.2 Hz, 1H), 6.95 (d, J=8.3 Hz, 1H), 6.38 (d, J=8.7 Hz, 1H), 6.12 (d, J=2.7 Hz, 1H), 4.91 (s, 1H), 4.32 (t, J=7.1 Hz, 2H), 3.37 (s, 1H), 3.30 (d, J=10.2 Hz, 1H), 2.96 (t, J=10.3 Hz, 1H), 2.20 (s, 1H), 2.09 (t, J=7.1 Hz, 2H), 1.86 (dd, J=11.9, 5.2 Hz, 1H), 1.61 (s, 4H), 1.55 (s, 3H), 1.47 (d, J=12.7 Hz, 4H), 1.04-0.94 (m, 2H), 0.94-0.85 (m, 2H). ESI-MS m/z calc. 709.2658, found 710.2 (M+1)⁺; Retention time: 2.06 minutes (LC method A).

### Example 132: Preparation of (14S)-12,12-Dimethyl-18-[(2H-1,2,3,4-tetrazol-5-yl)methyl]-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (Compound 372)

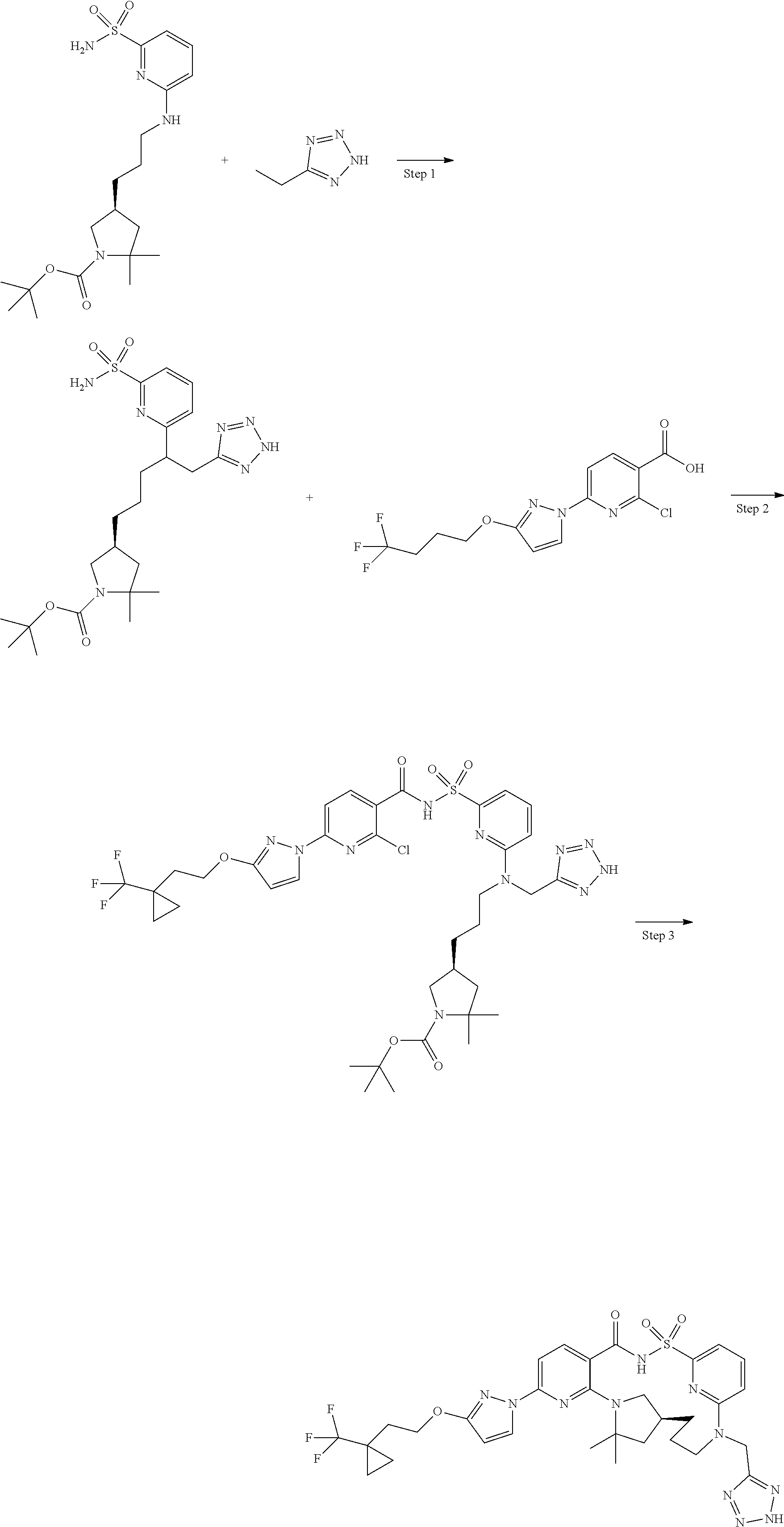

### Step 1: tert-Butyl (4S)-2,2-dimethyl-4-[3-[(6-sulfamoyl-2-pyridyl)-(1H-tetrazol-5-ylmethyl)amino]propyl]pyrrolidine-1-carboxylate

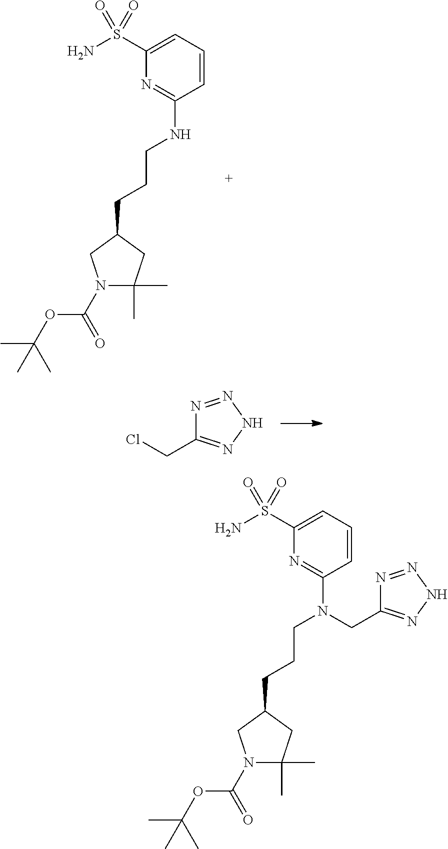

tert-Butyl (4S)-2,2-dimethyl-4-[3-[(6-sulfamoyl-2-pyridyl)amino]propyl]pyrrolidine-1-carboxylate (25 mg, 0.061 mmol) and NaH (8 mg of 60% w/w, 0.20 mmol) were combined in DMSO (750 μL) and stirred for 15 minutes. Then 5-(chloromethyl)-1H-tetrazole (8 mg, 0.067 mmol) was added and the mixture was allowed to stir for 90 min. Added 1eq additional 5-(chloromethyl)-1H-tetrazole (8 mg, 0.06750 mmol) and the mixture was allowed to stir for 16 hours. The mixture was filtered and then purified by reverse-phase preparative chromatography utilizing a C₁₈ column and HPLC-MS method 1-70% A1B1 (Acetonitrile-Water+5 mmolar HCl, 15 minute method) to afford a white solid as the desired tert-butyl (4S)-2,2-dimethyl-4-[3-[(6-sulfamoyl-2-pyridyl)-(1H-tetrazol-5-ylmethyl)amino]propyl]pyrrolidine-1-carboxylate (17.59 mg, 59%). ¹H NMR (500 MHz, DMSO-d₆) δ 7.73 (t, J=8.0 Hz, 1H), 7.18 (s, 2H), 7.11 (d, J=7.2 Hz, 1H), 6.95 (d, J=8.6 Hz, 1H), 5.12 (s, 2H), 3.55 (dt, J=17.4, 7.8 Hz, 3H), 2.86-2.69 (m, 1H), 2.08 (s, 1H), 1.99-1.76 (m, 1H), 1.58 (d, J=17.6 Hz, 2H), 1.39 (d, J=13.3 Hz, 9H), 1.35 (d, J=12.2 Hz, 5H), 1.30 (d, J=7.6 Hz, 1H), 1.23 (s, 3H), exchangeable NH of tetrazole not observed. ESI-MS m/z calc. 494.24237, found 495.2 (M+1)⁺; Retention time: 1.55 minutes (LC method A).

### Step 2: tert-Butyl (4S)-4-[3-[[6-[[2-chloro-6-[3-[2-[1-(trifluoromethyl)cyclopropyl] ethoxy]pyrazol-1-yl]pyridine-3-carbonyl]sulfamoyl]-2-pyridyl]-(2H-tetrazol-5-ylmethyl)amino]propyl]-2,2-dimethyl-pyrrolidine-1-carboxylate

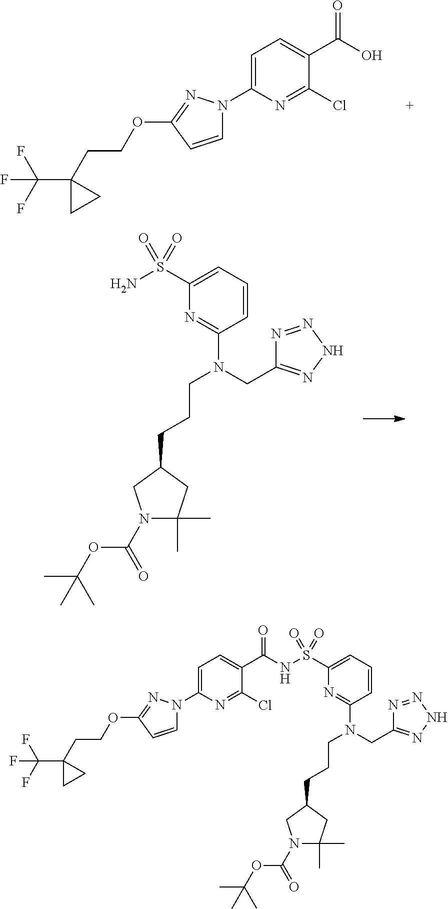 [SMILES: O=C(O)c1ccc(-n2ccc(OCCC3(C(F)(F)F)CC3)n2)nc1Cl ; CC(C)(C)OC(=O)N1C[C@@H](CCCN(Cc2nn[nH]n2)c2cccc(S(N)(=O)=O)n2)CC1(C)C ; CC(C)(C)OC(=O)N1C[C@@H](CCCN(Cc2nn[nH]n2)c2cccc(S(=O)(=O)NC(=O)c3ccc(-n4ccc(OCCC5(C(F)(F)F)CC5)n4)nc3Cl)n2)CC1(C)C]

To a solution of 2-chloro-6-[3-[2-[1-(trifluoromethyl)cyclopropyl]ethoxy] pyrazol-1-yl]pyridine-3-carboxylic acid (24 mg, 0.0634 mmol) in THF (500 μL) was added CDI (10 mg, 0.062 mmol) (recrystallized from THF) and the mixture was stirred at rt for 4h then tert-butyl (4S)-2,2-dimethyl-4-[3-[(6-sulfamoyl-2-pyridyl)-(2H-tetrazol-5-ylmethyl)amino]propyl]pyrrolidine-1-carboxylate (15 mg, 0.030 mmol) was added as a solution in THF (275 μL) followed by DBU (13 μL, 0.087 mmol) and the resulting mixture was stirred for 2 h at rt. The reaction mixture was filtered and then purified by reverse-phase preparative chromatography utilizing a C₁₈ column (HPLC-MS method 1-99% gradient, acetonitrile/Water+5 mmolar HCl, 15 minute run) to afford tert-butyl (4S)-4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]-(2H-tetrazol-5-ylmethyl)amino]propyl]-2,2-dimethyl-pyrrolidine-1-carboxylate as an off-white solid (11 mg, 43%). ESI-MS m/z calc. 851.29156, found 852.2 (M+1)⁺; Retention time: 2.21 minutes (LC method A).

### Step 3: (14S)-12,12-Dimethyl-18-[(2H-1,2,3,4-tetrazol-5-yl)methyl]-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (Compound 372)

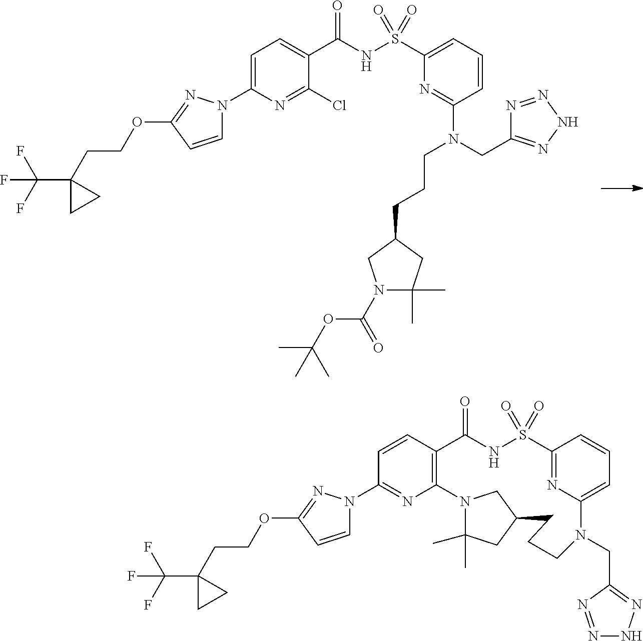 [SMILES: CC(C)(C)OC(=O)N1C[C@@H](CCCN(Cc2nn[nH]n2)c2cccc(S(=O)(=O)NC(=O)c3ccc(-n4ccc(OCCC5(C(F)(F)F)CC5)n4)nc3Cl)n2)CC1(C)C ; CC1(C)C[C@@H]2CCCN(Cc3nn[nH]n3)c3cccc(n3)S(=O)(=O)NC(=O)c3ccc(-n4ccc(OCCC5(C(F)(F)F)CC5)n4)nc3N1C2]

tert-Butyl (4S)-4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]-(2H-tetrazol-5-ylmethyl)amino]propyl]-2,2-dimethyl-pyrrolidine-1-carboxylate (11 mg, 0.01291 mmol) was dissolved in DCM (500 μL) and to the mixture was added HCl (4M in dioxane) (100 μL, 0.40 mmol) and the mixture was stirred at room temperature for 2 h. The mixture was evaporated to dryness, then placed on the high vacuum pump for 2 h to afford the intermediate 2-chloro-N-[[6-[3-[(3S)-5,5-dimethylpyrrolidin-3-yl]propyl-(2H-tetrazol-5-ylmethyl)amino]-2-pyridyl]sulfonyl]-6-[3-[2-[1-(trifluoromethyl)cyclopropyl]ethoxy] pyrazol-1-yl]pyridine-3-carboxamide (hydrochloride salt). The material was combined with K₂CO₃ (20 mg, 0.145 mmol), 3 Å molecular sieves and DMSO (500 μL) in a vial, which was purged with nitrogen, capped, heated to 155° C. and stirred for 18 h. The mixture was cooled to room temperature, filtered and concentrated under a stream of nitrogen to give a residue which was purified by reverse-phase preparative chromatography utilizing a C₁₈ column and HPLC-MS method with a 30-99% gradient (Acetonitrile-Water+5 mmolar HCl, 15 minute method) to afford as an off-white solid (14S)-12,12-dimethyl-18-[(2H-1,2,3,4-tetrazol-5-yl)methyl]-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(23),5(10),6,8,19,21-hexaene-2,2,4-trione (1.81 mg, 18%). ESI-MS m/z calc. 715.26245, found 716.2 (M+1)⁺; Retention time: 2.01 minutes (LC method A).

### Example 133: Preparation of (12S)-14,14-dimethyl-18-(3-{2-[1-(trifluoromethyl)cyclo propyl]ethoxy}-1H-pyrazol-1-yl)-24λ⁶-thia-2,5,6,7,8,15,17,23,29-noNaazapentacyclo [23.3.1.112,15.04,8.016,21]triaconta-1(28),4,6,16,18,20,25(29),26-octaene-22,24,24-trione (Compound 380)

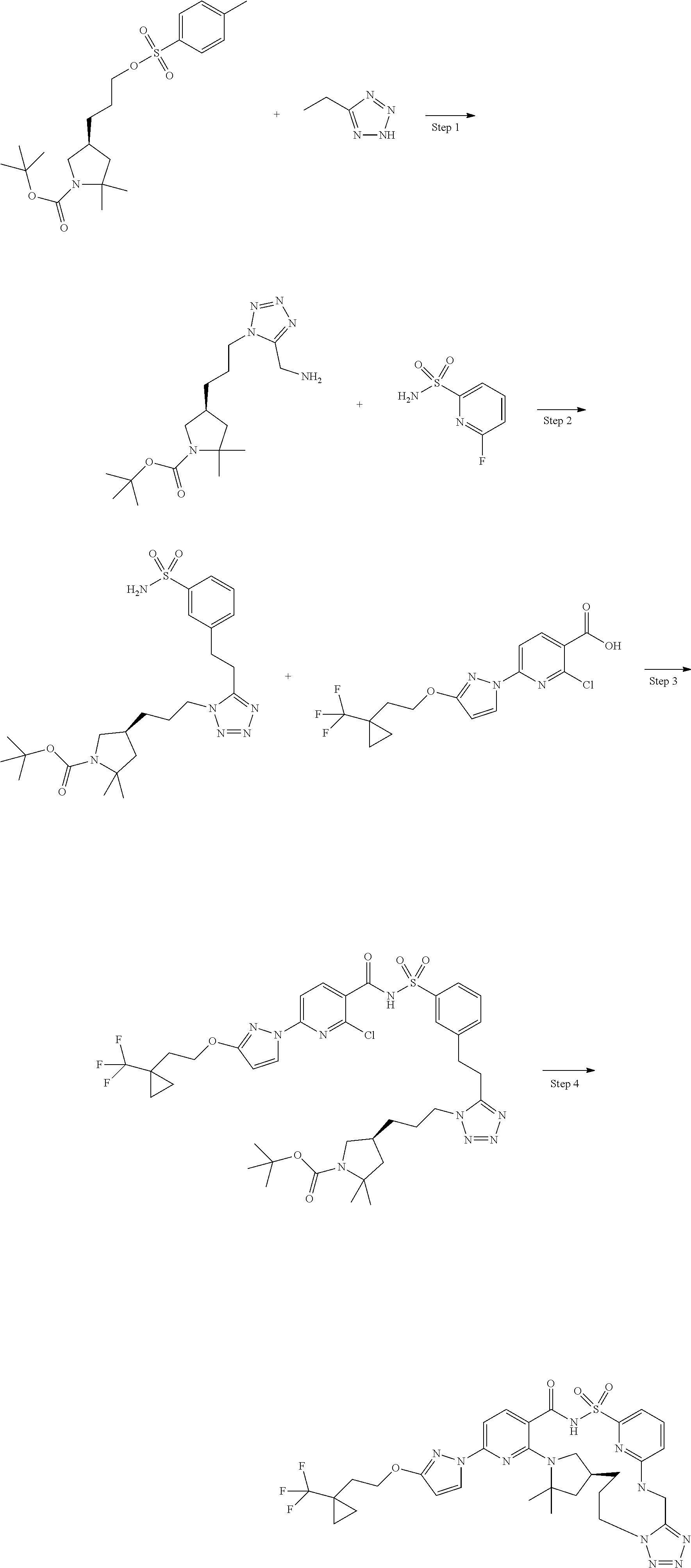

### Step 1: tert-Butyl (4S)-4-[3-[5-(aminomethyl)tetrazol-1-yl]propyl]-2,2-dimethyl-pyrrolidine-1-carboxylate

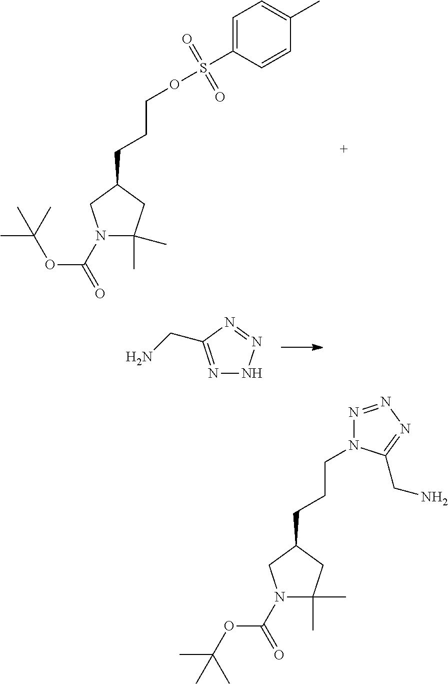

To a 40 mL sealed vial containing a mixture of tert-butyl (4S)-2,2-dimethyl-4-[3-(p-tolylsulfonyloxy)propyl]pyrrolidine-1-carboxylate (530 mg, 1.288 mmol) and 2H-tetrazol-5-ylmethanamine (155 mg, 1.564 mmol) in DMSO (15 mL) was added DIEA (350 μL, 2.01 mmol). The mixture was stirred at 85° C. for 2 h. The mixture was cooled to ambient temperature and saturated ammonium chloride was added and the mixture was extracted with ethyl acetate. The combined organic extracts washed with brine, dried over sodium sulfate, filtered and concentrated. The resulting colorless oil was purified utilizing silica gel column chromatography (4 gram column) using a gradient of 100% dichloromethane to 20% dichloromethane in methanol to afford tert-Butyl (4S)-4-[3-[5-(aminomethyl)tetrazol-1-yl]propyl]-2,2-dimethyl-pyrrolidine-1-carboxylate (318 mg, 73%) as a viscous oil. ESI-MS m/z calc. 338.243, found 339.2 (M+1)⁺; Retention time: 1.12 minutes (LC method A).

### Step 2: tert-Butyl (4S)-2,2-dimethyl-4-[3-[5-[[(6-sulfamoyl-2-pyridyl)amino]methyl]tetrazol-1-yl]propyl]pyrrolidine-1-carboxylate

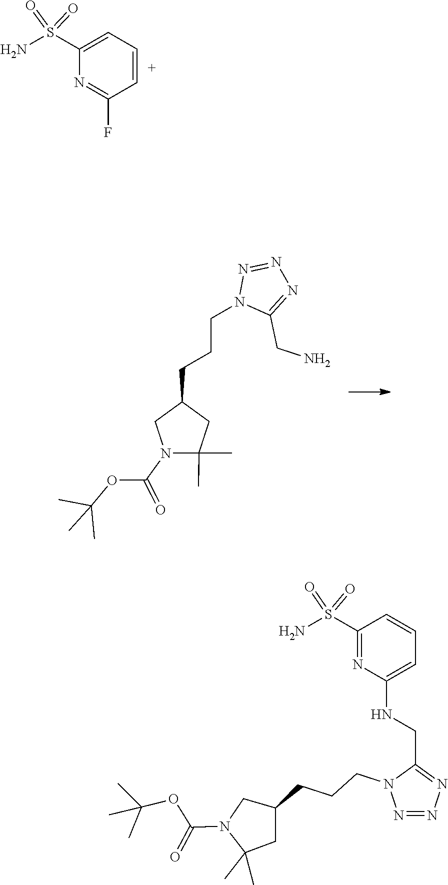 [SMILES: NS(=O)(=O)c1cccc(F)n1 ; CC(C)(C)OC(=O)N1C[C@@H](CCCn2nnnc2CN)CC1(C)C ; CC(C)(C)OC(=O)N1C[C@@H](CCCn2nnnc2CNc2cccc(S(N)(=O)=O)n2)CC1(C)C]

In a 150 mL sealed vessel was combined afford tert-Butyl (4S)-4-[3-[5-(aminomethyl)tetrazol-1-yl]propyl]-2,2-dimethyl-pyrrolidine-1-carboxylate (300 mg, 0.886 mmol) in DMSO (10.0 mL) followed by K₂CO₃ (650 mg, 4.703 mmol) and 6-fluoropyridine-2-sulfonamide (200 mg, 1.135 mmol). The vessel was sealed and heated to 95° C. on a hot plate for 72 hours. Added cesium carbonate (1.45 g, 4.450 mmol) and the mixture was heated to 105° C. for an additional 24 hours. The mixture was cooled to ambient temperature and saturated ammonium chloride was added and extracted with ethyl acetate. The combined organic extracts washed with brine, dried over sodium sulfate, filtered and concentrated. The resulting colorless oil was purified utilizing silica gel column chromatography (40 gram column) using a gradient of 100% dichloromethane to 20% dichloromethane in methanol followed by a second purification by reverse-phase preparative chromatography utilizing a C₁₈ column and HPLC-MS method (1-70% gradient Acetonitrile-Water+5 mmolar HCl, ×4 injections, 15 minute run) to afford tert-butyl (4S)-2,2-dimethyl-4-[3-[5-[[(6-sulfamoyl-2-pyridyl)amino]methyl]tetrazol-1-yl]propyl]pyrrolidine-1-carboxylate (165 mg, 38%) ESI-MS m/z calc. 494.24237, found 495.2 (M+1)⁺; Retention time: 1.55 minutes (LC method A).

### Step 3: tert-Butyl (4S)-4-[3-[5-[[[6-[[2-chloro-6-[3-[2-[1-(trifluoromethyl)cyclopropyl] ethoxy]pyrazol-1-yl]pyridine-3-carbonyl]sulfamoyl]-2-pyridyl]amino]methyl]tetrazol-1-yl]propyl]-2,2-dimethyl-pyrrolidine-1-carboxylate

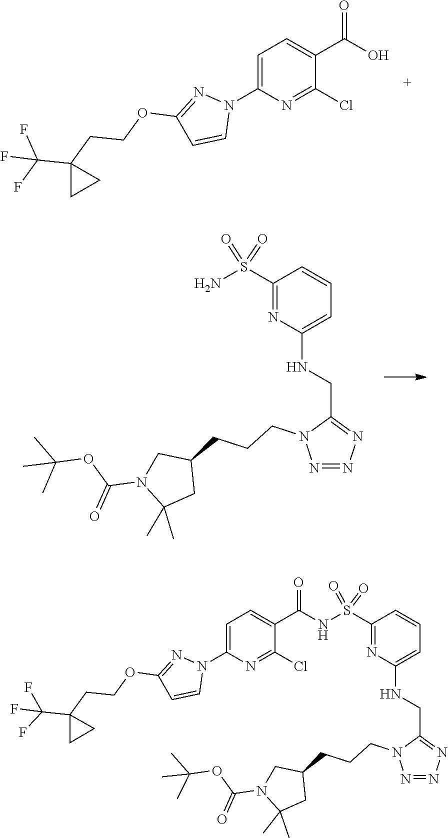 [SMILES: O=C(O)c1ccc(-n2ccc(OCCC3(C(F)(F)F)CC3)n2)nc1Cl ; CC(C)(C)OC(=O)N1C[C@@H](CCCn2nnnc2CNc2cccc(S(N)(=O)=O)n2)CC1(C)C ; CC(C)(C)OC(=O)N1C[C@@H](CCCn2nnnc2CNc2cccc(S(=O)(=O)NC(=O)c3ccc(-n4ccc(OCCC5(C(F)(F)F)CC5)n4)nc3Cl)n2)CC1(C)C]

To a solution of 2-chloro-6-[3-[2-[1-(trifluoromethyl)cyclopropyl]ethoxy]pyrazol-1-yl]pyridine-3-carboxylic acid (168 mg, 0.447 mmol) in THF (3.5 mL) was added CDI (75 mg, 0.462 mmol) (recrystallized from THF) and the mixture was stirred at rt for 4 h then tert-butyl (4S)-2,2-dimethyl-4-[3-[5-[[(6-sulfamoyl-2-pyridyl)amino]methyl]tetrazol-1-yl]propyl]pyrrolidine-1-carboxylate (100 mg, 0.202 mmol) was added as a solution in THF (1.5 mL), followed by DBU (110 μL, 0.736 mmol) and the resulting mixture was stirred for 18 h at rt. The reaction mixture was filtered and then purified by reverse-phase preparative chromatography utilizing a C₁₈ column and HPLC-MS method (30-99% gradient acetonitrile-Water+5 mmolar HCl×2 injections, 15 minute run) to afford tert-butyl (4S)-4-[3-[5-[[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]methyl]tetrazol-1-yl]propyl]-2,2-dimethyl-pyrrolidine-1-carboxylate as an off-white solid (44.90 mg, 26%). ESI-MS m/z calc. 851.29156, found 852.2 (M+1)⁺; Retention time: 2.21 minutes (LC method A).

### Step 4: (12S)-14,14-Dimethyl-18-(3-{2-[1-(trifluoromethyl)cyclopropyl]ethoxy}-1H-pyrazol-1-yl)-24λ⁶-thia-2,5,6,7,8,15,17,23,29-noNaazapentacyclo [23.3.1.112,15.04,8.016,21]triaconta-1(28),4,6,16,18,20,25(29),26-octaene-22,24,24-trione (Compound 380)

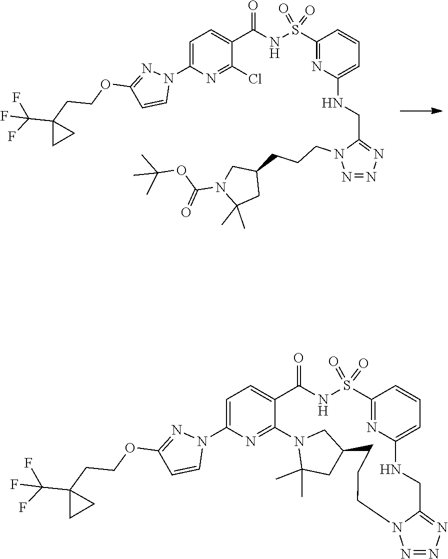 [SMILES: CC(C)(C)OC(=O)N1C[C@@H](CCCn2nnnc2CNc2cccc(S(=O)(=O)NC(=O)c3ccc(-n4ccc(OCCC5(C(F)(F)F)CC5)n4)nc3Cl)n2)CC1(C)C ; CC1(C)C[C@@H]2CCCn3nnnc3CNc3cccc(n3)S(=O)(=O)NC(=O)c3ccc(-n4ccc(OCCC5(C(F)(F)F)CC5)n4)nc3N1C2]

tert-Butyl (4S)-4-[3-[5-[[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]methyl] tetrazol-1-yl]propyl]-2,2-dimethyl-pyrrolidine-1-carboxylate (44.9 mg, 0.0527 mmol) was dissolved in DCM (1.5 mL) and to the mixture was added HCl (4M in dioxane) (400 μL of 4 M, 1.600 mmol) and the mixture was stirred at room temperature for 2h. The mixture was evaporated to dryness, then placed on the high vacuum pump for 2 h to afford the intermediate 2-chloro-N-[[6-[[1-[3-[(3S)-5,5-dimethylpyrrolidin-3-yl]propyl]tetrazol-5-yl]methylamino]-2-pyridyl]sulfonyl]-6-[3-[2-[1-(trifluoromethyl)cyclopropyl]ethoxy]pyrazol-1-yl]pyridine-3-carboxamide (hydrochloride salt) as an off-white solid. ESI-MS m/z calc. 751.23914, found 752.2 (M+1)⁺; Retention time: 0.76 minutes. The material was combined with K₂CO₃ (75 mg, 0.543 mmol), 3 Å molecular sieves and DMSO (1.5 mL) in a vial, which was purged with nitrogen, capped, heated to 155° C. and stirred for 16 h. The mixture was filtered and concentrated under a stream of nitrogen to give a residue which was purified by reverse-phase preparative chromatography utilizing a C₁₈ column and HPLC-MS method (30-99% gradient, acetonitrile-Water+5 mmolar HCl, 15 minute method) to afford as an off-white solid, (12S)-14,14-dimethyl-18-(3-{2-[1-(trifluoromethyl)cyclopropyl]ethoxy}-1H-pyrazol-1-yl)-24λ⁶-thia-2,5,6,7,8,15,17,23,29-noNaazapentacyclo[23.3.1.112,15.04,8.016,21] triaconta-1(28),4,6,16,18,20,25(29),26-octaene-22,24,24-trione (15.6 mg, 41%). ESI-MS m/z calc. 715.26245, found 716.2 (M+1)⁺; Retention time: 1.97 minutes (LC method A).

### Example 134: Preparation of (14S)-17-(4-tert-butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 1, (Compound 300) and (14S)-17-(4-tert-butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 2 (Compound 299)

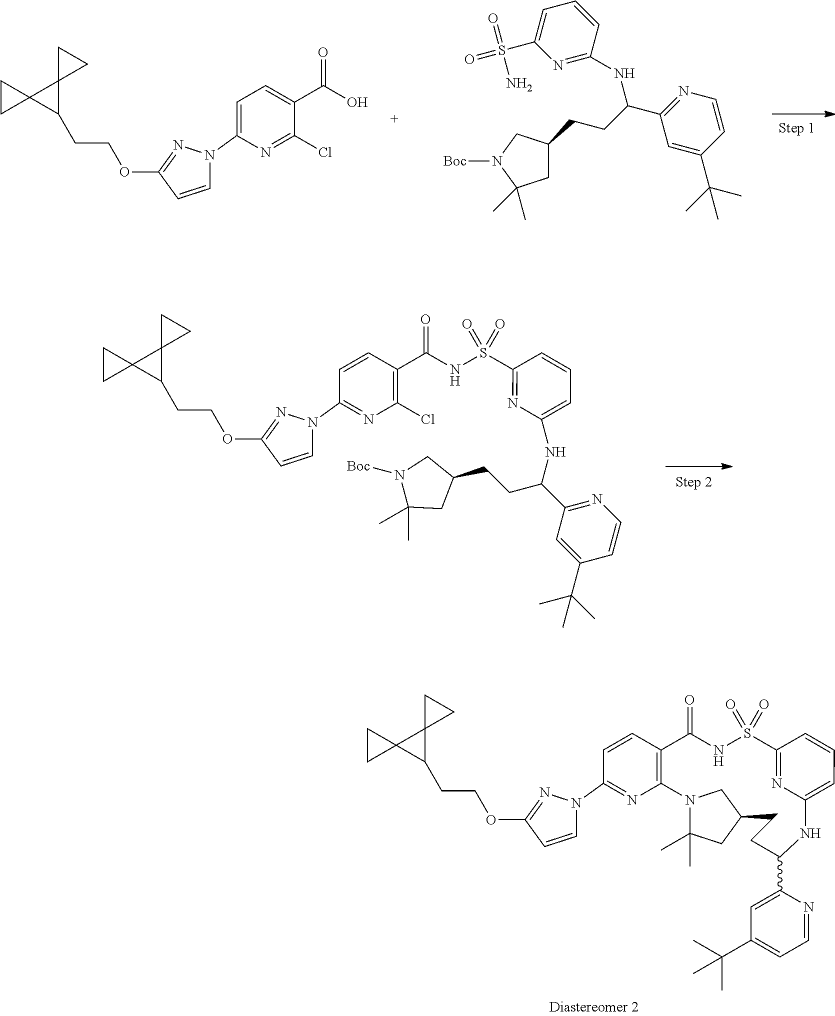 [SMILES: O=C(O)c1ccc(-n2ccc(OCCC3C4(CC4)C34CC4)n2)nc1Cl ; CC(C)(C)OC(=O)N1C[C@@H](CCC(Nc2cccc(S(N)(=O)=O)n2)c2cc(C(C)(C)C)ccn2)CC1(C)C ; CC(C)(C)OC(=O)N1C[C@@H](CCC(Nc2cccc(S(=O)(=O)NC(=O)c3ccc(-n4ccc(OCCC5C6(CC6)C56CC6)n4)nc3Cl)n2)c2cc(C(C)(C)C)ccn2)CC1(C)C ; CC(C)(C)c1ccnc(C2CC[C@@H]3CN(c4nc(-n5ccc(OCCC6C7(CC7)C67CC7)n5)ccc4C(=O)NS(=O)(=O)c4cccc(n4)N2)C(C)(C)C3)c1]

### Step 1: tert-Butyl (4S)-4-[3-(4-tert-butyl-2-pyridyl)-3-[[6-[[2-chloro-6-[3-(2-dispiro[2.0.24.13]heptan-7-ylethoxy)pyrazol-1-yl]pyridine-3-carbonyl]sulfamoyl]-2-pyridyl]amino]propyl]-2,2-dimethyl-pyrrolidine-1-carboxylate

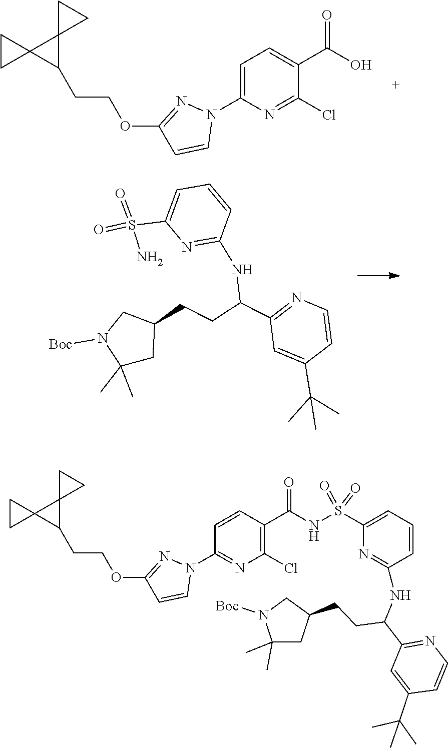

In a 20-mL vial, 2-chloro-6-[3-(2-dispiro[2.0.24.13]heptan-7-ylethoxy)pyrazol-1-yl]pyridine-3-carboxylic acid (178.8 mg, 0.447 mmol) was dissolved in THF (3.0 mL), to which CDI (120.5 mg, 0.743 mmol) was added. The resulting mixture was stirred at room temperature for 15 h. After this time, tert-butyl (4S)-4-[3-(4-tert-butyl-2-pyridyl)-3-[(6-sulfamoyl-2-pyridyl)amino]propyl]-2,2-dimethyl-pyrrolidine-1-carboxylate (300 mg, 0.5497 mmol) and DBU (300 μL, 2.006 mmol) were added, and the resulting mixture was stirred at room temperature for 24 h. After this time, aqueous HCl solution (1 N, 4 mL) was added, and the mixture was extracted with EtOAc (3×3 mL). The combined organic extracts was washed with H₂O (5 mL) and saturated aqueous NaCl solution (5 mL), then dried over Na₂SO₄, filtered, and evaporated in vacuo to give an orange foam. Purification by silica gel chromatography (12 g of silica) using a gradient eluent of 0 to 80% EtOAc in hexanes gave a mixture of diastereomers of tert-butyl (4S)-4-[3-(4-tert-butyl-2-pyridyl)-3-[[6-[[2-chloro-6-[3-(2-dispiro[2.0.24.13]heptan-7-ylethoxy)pyrazol-1-yl]pyridine-3-carbonyl]sulfamoyl]-2-pyridyl]amino]propyl]-2,2-dimethyl-pyrrolidine-1-carboxylate (173.0 mg, 44%) ESI-MS m/z calc. 886.39667, found 887.5 (M+1)⁺; Retention time: 2.19 minutes (LC method A).

### Step 2: (14S)-17-(4-tert-Butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 1, (Compound 300) and (14S)-17-(4-tert-butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 2 (Compound 299)

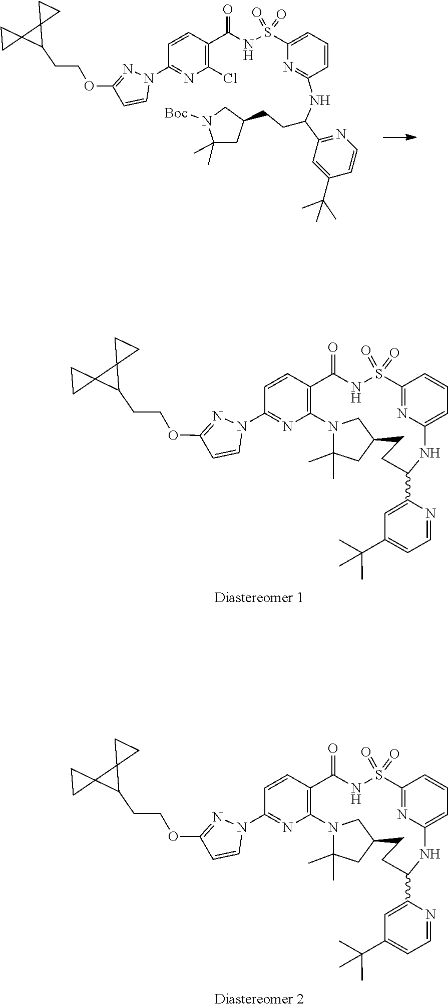 [SMILES: CC(C)(C)OC(=O)N1C[C@@H](CCC(Nc2cccc(S(=O)(=O)NC(=O)c3ccc(-n4ccc(OCCC5C6(CC6)C56CC6)n4)nc3Cl)n2)c2cc(C(C)(C)C)ccn2)CC1(C)C ; CC(C)(C)c1ccnc(C2CC[C@@H]3CN(c4nc(-n5ccc(OCCC6C7(CC7)C67CC7)n5)ccc4C(=O)NS(=O)(=O)c4cccc(n4)N2)C(C)(C)C3)c1 ; CC(C)(C)c1ccnc(C2CC[C@@H]3CN(c4nc(-n5ccc(OCCC6C7(CC7)C67CC7)n5)ccc4C(=O)NS(=O)(=O)c4cccc(n4)N2)C(C)(C)C3)c1]

Stage 1: In a 20-mL vial, tert-butyl (4S)-4-[3-(4-tert-butyl-2-pyridyl)-3-[[6-[[2-chloro-6-[3-(2-dispiro[2.0.24.13]heptan-7-ylethoxy)pyrazol-1-yl]pyridine-3-carbonyl]sulfamoyl]-2-pyridyl]amino]propyl]-2,2-dimethyl-pyrrolidine-1-carboxylate (173.0 mg, 0.195 mmol) was dissolved in CH₂Cl₂ (4.0 mL). TFA (1.0 mL, 12.98 mmol) was added, and the resulting solution was allowed to stand at room temperature for 3 h. The mixture was then evaporated in vacuo, diluted with dioxane, and evaporated in vacuo again. This gave an orange oil, 300 mg (>100% yield).

Stage 2: In a 20-mL microwave vial with a pressure relief septum cap, the crude product from stage 1 was dissolved in NMP (4.0 mL), to which K₂CO₃ (285.9 mg, 2.069 mmol) was added. The resulting mixture was flushed with N₂, and then stirred at 140° C. for 18 h. After cooling to room temperature, the reaction mixture was quenched with 1 N HCl solution (5 mL) and extracted with EtOAc (3×3 mL). The combined organic extracts was washed with H₂O (5 mL) and saturated aqueous NaCl solution (5 mL), then dried over Na₂SO₄, filtered, and evaporated in vacuo. Purification by silica gel chromatography (12 g of silica) using a gradient eluent of 0 to 80% EtOAc in hexanes gave an off-white powder, (65.2 mg, 44.5% yield), but this contained both diastereomers. Further purification by reverse-phase preparative chromatography using a C₁₈ column and a gradient eluent of 1 to 99% MeCN in H₂O containing 5 mM HCl gave two products:

Diastereomer 1: “Peak 1”, (14S)-17-(4-tert-butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (19.0 mg, 13%); ¹H NMR (400 MHz, DMSO-d₆) δ 12.74-12.01 (bs, 1H), 8.66-8.54 (m, 1H), 8.20 (d, J=2.7 Hz, 1H), 8.05-7.83 (m, 2H), 7.77-7.56 (m, 3H), 7.36-7.24 (m, 1H), 6.99-6.91 (m, 1H), 6.91 (d, J=8.2 Hz, 1H), 6.08 (d, J=2.7 Hz, 1H), 5.24-4.99 (m, 1H), 4.21 (t, J=6.6 Hz, 2H), 3.09-2.77 (m, 2H), 2.39-2.27 (m, 1H), 2.24-2.11 (m, 1H), 1.91 (dd, J=11.9, 5.7 Hz, 1H), 1.85-1.71 (m, 3H), 1.58 (s, 3H), 1.55 (s, 3H), 1.54-1.48 (m, 1H), 1.47 (t, J=6.4 Hz, 1H), 1.30 (s, 9H), 1.20-1.08 (m, 1H), 0.88-0.77 (m, 4H), 0.68-0.59 (m, 2H), 0.54-0.45 (m, 2H). [Note: 1H is missing, presumably hidden under the water peak.]. ESI-MS m/z calc. 750.36755, found 751.4 (M+1)⁺; Retention time: 2.05 minutes (LC method A).

Diastereomer 2: “Peak 2”, (14S)-17-(4-tert-butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (27.8 mg, 19%); ¹H NMR (400 MHz, DMSO-d₆) δ 12.97-11.36 (bs, 1H), 8.73-8.57 (m, 1H), 8.22 (d, J=2.8 Hz, 1H), 8.15-8.05 (m, 1H), 8.05-7.90 (m, 1H), 7.87 (d, J=8.2 Hz, 1H), 7.80-7.71 (m, 1H), 7.71 (t, J=7.9 Hz, 1H), 7.19 (d, J 7.2 Hz, 1H), 6.97 (d, J=8.4 Hz, 1H), 6.94 (d, J=8.2 Hz, 1H), 6.10 (d, J=2.8 Hz, 1H), 5.59-5.40 (m, 1H), 4.22 (t, J=6.6 Hz, 2H), 2.71 (t, J=10.4 Hz, 1H), 2.42-2.28 (m, 1H), 2.12-1.93 (m, 2H), 1.89 (dd, J=12.0, 5.3 Hz, 1H), 1.85-1.79 (m, 2H), 1.79-1.73 (m, 1H), 1.66 (s, 3H), 1.65-1.56 (m, 1H), 1.54 (s, 3H), 1.47 (t, J=6.5 Hz, 2H), 1.32 (s, 9H), 0.91-0.74 (m, 4H), 0.70-0.58 (m, 2H), 0.54-0.42 (m, 2H). [Note: 1H is missing, presumably hidden under the water peak.] ESI-MS m/z calc. 750.36755, found 751.3 (M+1)⁺; Retention time: 2.04 minutes (LC method A).

### Example 135: Preparation of 12-methyl-12-propyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 1 (Compound 368), enantiomer 2 (Compound 367), enantiomer 3 (Compound 366), and enantiomer 4 (Compound 365)

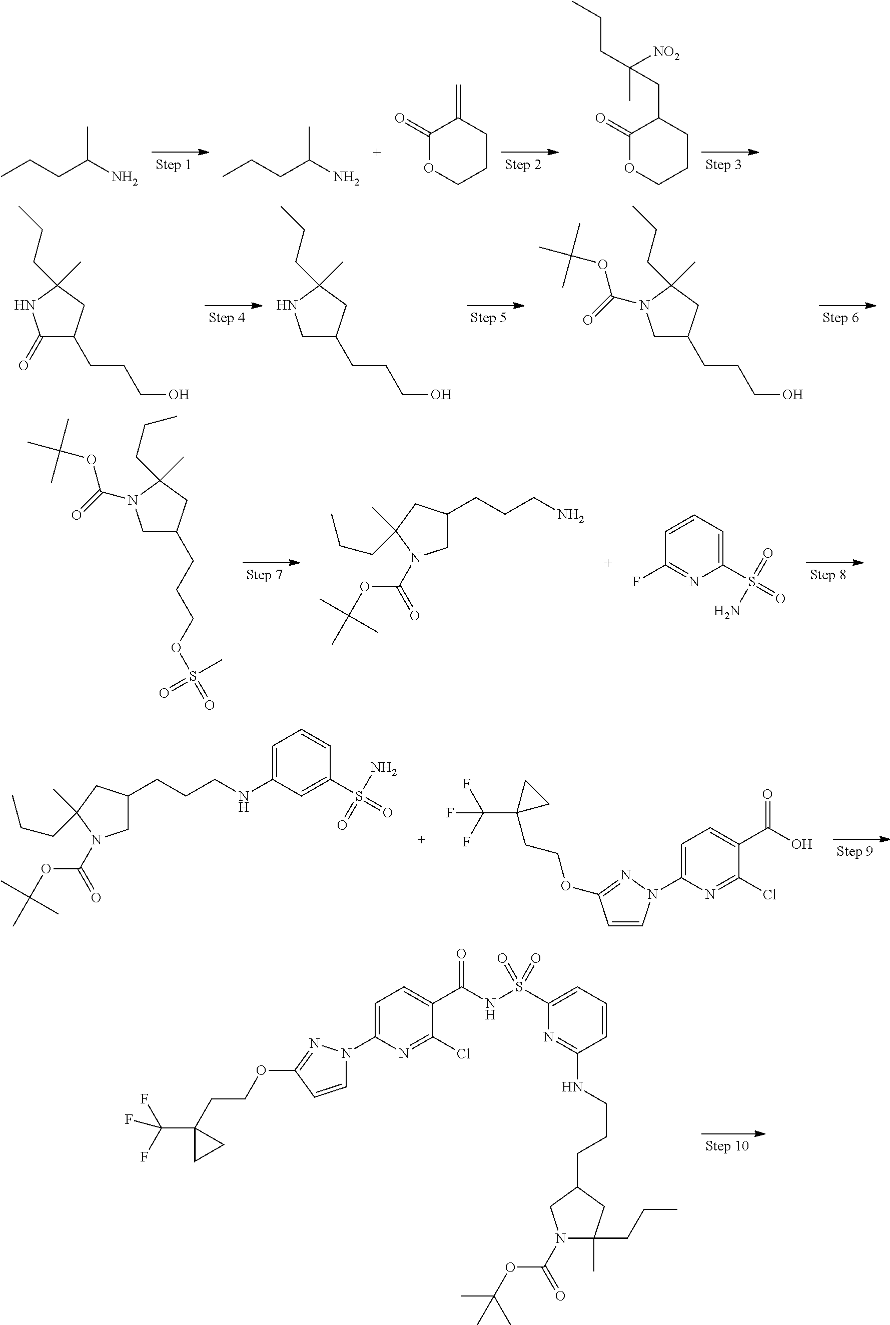

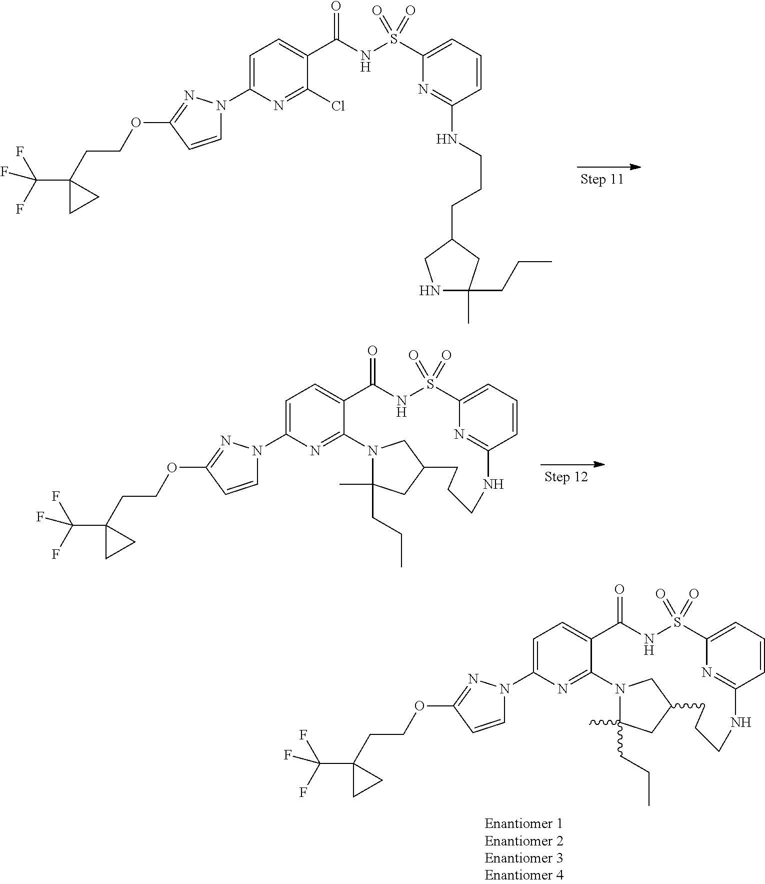

### Step 1: 2-Nitropentane

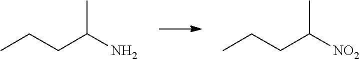

To a 2 L 3-necked flask equipped with condenser was added 3-Chloroperoxybenzoic acid (184.07 g, 800.00 mmol) in 1,2-dichloroethane (1.2 L) and the mixture was heated to 85° C. Pentan-2-amine (17.970 g, 199.98 mmol) in 1,2-Dichloroethane (100 mL) was added dropwise. The mixture was stirred 3 hours at 85° C. After cooling down to rt, the reaction mixture was filtered and washed with 1 N NaOH (3×1.5 L) and dried over Na₂SO₄. The organic layer was evaporated to afford crude 2-nitropentane which was combined with previous 4 batches and purified by distillation to afford 2-nitropentane as a yellow oil (9.61 g, 39%). ¹H NMR (250 MHz, CDCl₃) δ 4.78-4.35 (m, 1H), 2.11-1.88 (m, 1H), 1.66 (ddt, J=14.1, 8.9, 4.6 Hz, 1H), 1.51 (d, J=6.6 Hz, 3H), 1.33 (dt, J=15.1, 7.6 Hz, 2H), 0.93 (t, J=7.3 Hz, 3H)

### Step 2: 3-(2-Methyl-2-nitro-pentyl)tetrahydropyran-2-one

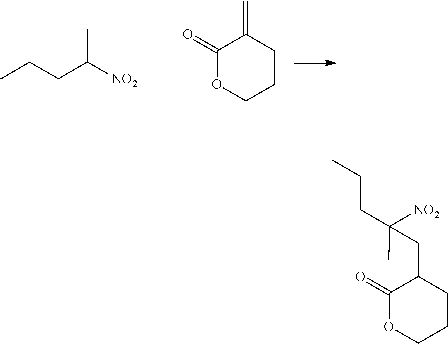

To a solution of 2-nitropentane (24.9 g, 201.93 mmol) and 3-methylenetetrahydropyran-2-one (17.02 g, 127.51 mmol) was added 1,8-Diazabicyclo[5.4.0]undec-7-ene (2.3 g, 14.806 mmol) dropwise. The mixture was stirred at rt for 2h. The reaction mixture was diluted with DCM (200 mL) and washed with 5% HCL (2×5 mL). The organic layer was evaporated to afford a crude material which was purified by chromatography on a 220 g silica gel to afford 3-(2-methyl-2-nitro-pentyl)tetrahydropyran-2-one as a light yellow oil (26.1 g, 85%). ¹H NMR (250 MHz, CDCl₃) δ 4.39-4.21 (m, 2H), 2.89-2.29 (m, 2H), 2.25-1.69 (m, 6H), 1.60-1.53 (m, 3H), 1.50-1.04 (m, 3H), 1.01-0.82 (m, 3H).

### Step 3: 3-(3-Hydroxypropyl)-5-methyl-5-propyl-pyrrolidin-2-one

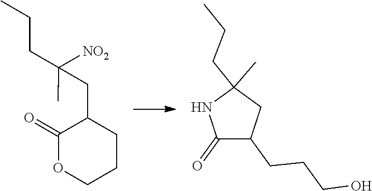 [SMILES: CCCC(C)(CC1CCCOC1=O)[N+](=O)[O-] ; CCCC1(C)CC(CCCO)C(=O)N1]

To a solution of 3-(2-methyl-2-nitro-pentyl)tetrahydropyran-2-one (12 g, 43.965 mmol) in EtOAc (50 mL) was added Raney-Ni (5 g, 32.187 mmol) and the mixture was stirred under H₂ atmosphere (˜10 bar) at 80° C. overnight. The reaction mixture was filtered. The filtrate was evaporated to provide 3-(3-hydroxypropyl)-5-methyl-5-propyl-pyrrolidin-2-one (8 g, 91%) as light yellow oil which was directly used for the next step without further purification. ESI-MS m/z calc. 199.1572, found 200.6 (M+1)⁺; Retention time: 2.17 minutes (LC method B).

### Step 4: 3-(5-Methyl-5-propyl-pyrrolidin-3-yl)propan-1-ol

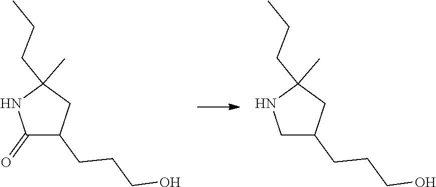

To a solution of 3-(3-hydroxypropyl)-5-methyl-5-propyl-pyrrolidin-2-one (7.98 g, 38.040 mmol) in dry tetrahydrofuran (200 mL) under reflux, 2M borane dimethyl sulfide complex in THF (150 mL, 300 mmol) was added dropwise. After 1 hour, the mixture was cooled at 0° C. and 150 mL of a solution of MeOH/HCl (9:1) were slowly added. The mixture was stirred at 55° C. for 20 hours, allowed to cool to ambient temperature and concentrated under reduced pressure. The residue was diluted with saturated sodium bicarbonate (200 mL) and extracted with dichloromethane (2×150 mL). The product in the aqueous layer was not isolated, but directly used in the next BOC protection step. ESI-MS m/z calc. 185.17796, found 186.5 (M+1)⁺; Retention time: 1.49 minutes (LC method B).

### Step 5: tert-Butyl 4-(3-hydroxypropyl)-2-methyl-2-propyl-pyrrolidine-1-carboxylate

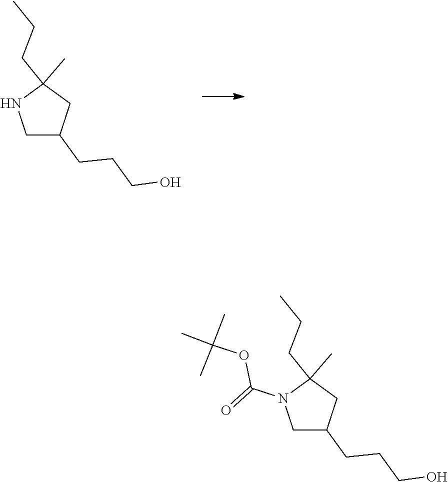 [SMILES: CCCC1(C)CC(CCCO)CN1 ; CCCC1(C)CC(CCCO)CN1C(=O)OC(C)(C)C]

To a solution of 3-(5-methyl-5-propyl-pyrrolidin-3-yl)propan-1-ol (7.049 g, 38.040 mmol) in saturated NaHCO₃(200 mL), 2M NaOH (50 mL) and Dioxane (80 mL) was added BOC₂O (16.604 g, 76.080 mmol). The mixture was stirred at room temperature for 1 hour. The mixture was extracted with ethyl acetate (3×200 mL). The organic layer was washed with brine (500 mL), dried over sodium sulfate and concentrated. The residue was purified by silica gel column chromatography using 0-70% hexanes-ethyl acetate to afford tert-butyl 4-(3-hydroxypropyl)-2-methyl-2-propyl-pyrrolidine-1-carboxylate (2.13 g, 19%) as a colorless gel. ESI-MS m/z calc. 285.2304, found 286.5 (M+1)⁺; Retention time: 3.26 minutes (LC method B).

### Step 6: tert-Butyl 2-methyl-4-(3-methylsulfonyloxypropyl)-2-propyl-pyrrolidine-1-carboxylate

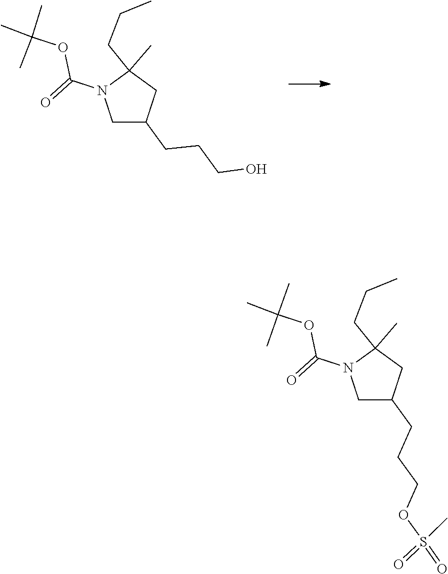 [SMILES: CCCC1(C)CC(CCCO)CN1C(=O)OC(C)(C)C ; CCCC1(C)CC(CCCOS(C)(=O)=O)CN1C(=O)OC(C)(C)C]

To a solution of tert-butyl 4-(3-hydroxypropyl)-2-methyl-2-propyl-pyrrolidine-1-carboxylate (2.13 g, 7.3134 mmol) and TEA (1.4520 g, 2 mL, 14.349 mmol) in DCM (20 mL) was added MsCl (1.4800 g, 1.0000 mL, 12.881 mmol) dropwise at −5° C. The mixture was stirred at 0° C. for 1 hour. The mixture was diluted with water (20 mL) and the two layers separated. The aqueous layer was extracted with DCM (2×50 mL). The combined organic layers were washed with water (50 mL) and brine (80 mL), dried over sodium sulfate, filtered and concentrated to afford tert-butyl 2-methyl-4-(3-methylsulfonyloxypropyl)-2-propyl-pyrrolidine-1-carboxylate (2.7 g, 100%) as a light yellow oil. ESI-MS m/z calc. 363.2079, found 364.5 (M+1)⁺; Retention time: 3.52 minutes (LC method B).

### Step 7: tert-Butyl 4-(3-aminopropyl)-2-methyl-2-propyl-pyrrolidine-1-carboxylate

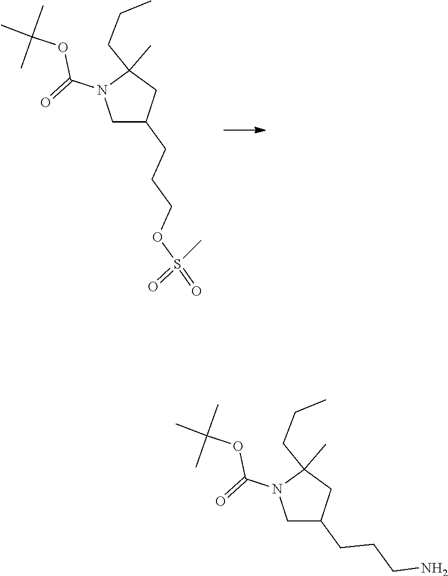 [SMILES: CCCC1(C)CC(CCCOS(C)(=O)=O)CN1C(=O)OC(C)(C)C ; CCCC1(C)CC(CCCN)CN1C(=O)OC(C)(C)C]

To a solution of tert-butyl 2-methyl-4-(3-methylsulfonyloxypropyl)-2-propyl-pyrrolidine-1-carboxylate (2.7 g, 7.2790 mmol) in NMP (20 mL) was added NaN₃ (0.55 g, 8.4179 mmol) and the mixture stirred at 45° C. for 18 hours. The mixture was diluted with water (50 mL) and followed by saturated sodium bicarbonate (50 mL) and the mixture extracted with ethyl acetate (2×80 mL). The organic layer was washed with brine (150 mL), dried over sodium sulfate, filtered and concentrated to give an intermediate azide. To a solution of this azide intermediate in EtOAc (20 mL) was added Platinum oxide monohydrate (0.35 g, 1.5105 mmol). The mixture was in a Parr shaker at 40 psi of hydrogen for 2 hours. The reaction mixture was filtered through a Celite pad. The filtrate was concentrated to afford tert-butyl 4-(3-aminopropyl)-2-methyl-2-propyl-pyrrolidine-1-carboxylate (2 g, 95%) as a dark oil. ESI-MS m/z calc. 284.2464, found 285.6 (M+1)⁺; Retention time: 2.57 minutes (LC method B).

### Step 8: tert-Butyl 2-methyl-2-propyl-4-[3-[(6-sulfamoyl-2-pyridyl)amino]propyl]pyrrolidine-1-carboxylate

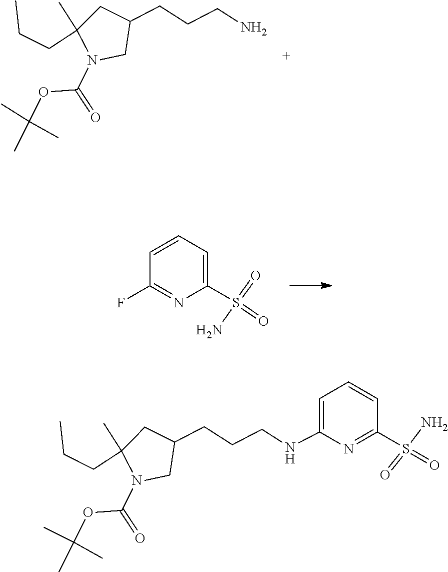 [SMILES: CCCC1(C)CC(CCCN)CN1C(=O)OC(C)(C)C ; NS(=O)(=O)c1cccc(F)n1 ; CCCC1(C)CC(CCCNc2cccc(S(N)(=O)=O)n2)CN1C(=O)OC(C)(C)C]

To a mixture of tert-butyl 4-(3-aminopropyl)-2-methyl-2-propyl-pyrrolidine-1-carboxylate (2 g, 6.8908 mmol) and 6-fluoropyridine-2-sulfonamide (1.5 g, 8.0888 mmol) in DMSO (10 mL) was added DIEA (2.2260 g, 3 mL, 17.223 mmol). The mixture was stirred at 110° C. for 16 hours. The mixture was cooled to room temperature and the mixture was directly load to prep.-HPLC column to purify (column: Varian C₁₈ 10 μm, 5×30 cm; flow rate: 60 mL/min.; mobile phase A: water; mobile phase B: acetonitrile; method: 0-45% B over 60 minutes). The pure product fractions were combined and acetonitrile was removed. The cloudy water phase was extracted with ethyl acetate (2×200 mL). The organic layer was washed by brine (250 mL), dried over sodium sulfate and concentrated to afford tert-butyl 2-methyl-2-propyl-4-[3-[(6-sulfamoyl-2-pyridyl)amino]propyl]pyrrolidine-1-carboxylate (2.58 g, 82%) as a white solid. ¹H NMR (500 MHz, DMSO-d₆) δ 7.55-7.46 (m, 1H), 7.07 (s, 2H), 6.94 (d, J=7.0 Hz, 2H), 6.60 (d, J=8.4 Hz, 1H), 3.62-3.51 (m, 1H), 3.30-3.24 (m, 2H), 2.86-2.63 (m, 1H), 2.09-2.02 (m, 1H), 1.80-1.45 (m, 5H), 1.43-1.32 (m, 12H), 1.29 (d, J 11.9 Hz, 2H), 1.25-1.20 (m, 2H), 1.18-1.00 (m, 1H), 0.91-0.80 (m, 3H). ESI-MS m/z calc. 440.2457, found 441.1 (M+1)⁺; Retention time: 2.7 minutes (LC method H).

### Step 9: tert-Butyl 4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]propyl]-2-methyl-2-propyl-pyrrolidine-1-carboxylate

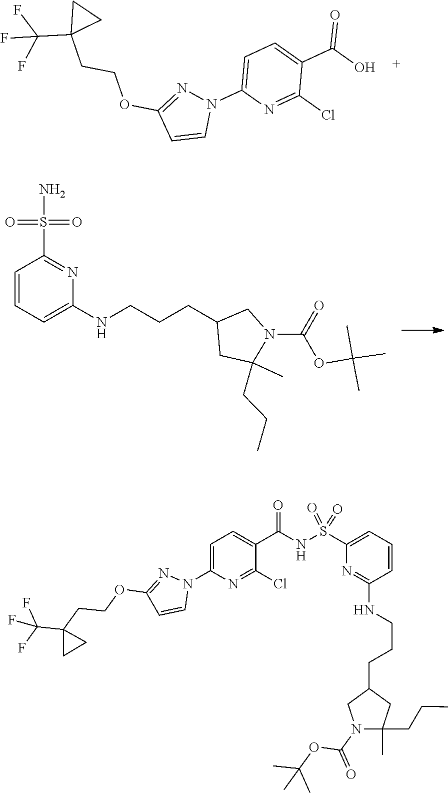 [SMILES: O=C(O)c1ccc(-n2ccc(OCCC3(C(F)(F)F)CC3)n2)nc1Cl ; CCCC1(C)CC(CCCNc2cccc(S(N)(=O)=O)n2)CN1C(=O)OC(C)(C)C ; CCCC1(C)CC(CCCNc2cccc(S(=O)(=O)NC(=O)c3ccc(-n4ccc(OCCC5(C(F)(F)F)CC5)n4)nc3Cl)n2)CN1C(=O)OC(C)(C)C]

To a solution of the 2-chloro-6-[3-[2-[1-(trifluoromethyl)cyclopropyl]ethoxy]pyrazol-1-yl]pyridine-3-carboxylic acid (400 mg, 1.065 mmol) in THF (3.2 mL) was added CDI (215.7 mg, 1.330 mmol) (recrystallized from THF) and the mixture was stirred at rt for 1 h then tert-butyl 2-methyl-2-propyl-4-[3-[(6-sulfamoyl-2-pyridyl)amino]propyl]pyrrolidine-1-carboxylate (490.2 mg, 1.113 mmol) was added followed by DBU (509.1 mg, 3.344 mmol) and the resulting mixture was stirred for 16 h at room temperature. Concentrated to remove the THF then diluted with DMSO, filtered and purified using a reverse-phase 275 g C₁₈ column using a gradient from 50% ACN/H₂O to 99% ACN/H₂O. Product did not separate well from the starting sulfonamide. The impure product was dissolved in DMSO, filtered and further purified using a reverse phase HPLC-MS method using a Luna C₁₈ (2) column (75×30 mm, 5 μm particle size) sold by Phenomenex (pn: 00C-4252-U0-AX), and a dual gradient run from 1-99% mobile phase B over 15.0 minutes (mobile phase A=H₂O (5 mM HCl), mobile phase B=acetonitrile, flow rate=50 mL/min, injection volume=950 μL and column temperature=25° C.) giving tert-butyl 4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]propyl]-2-methyl-2-propyl-pyrrolidine-1-carboxylate as a white solid (533.3 mg, 63%). ESI-MS m/z calc. 797.2949, found 798.3 (M+1)⁺; Retention time: 0.91 minutes (LC method D).

### Step 10: 2-Chloro-N-[[6-[3-(5-methyl-5-propyl-pyrrolidin-3-yl)propylamino]-2-pyridyl]sulfonyl]-6-[3-[2-[1-(trifluoromethyl)cyclopropyl]ethoxy]pyrazol-1-yl]pyridine-3-carboxamide

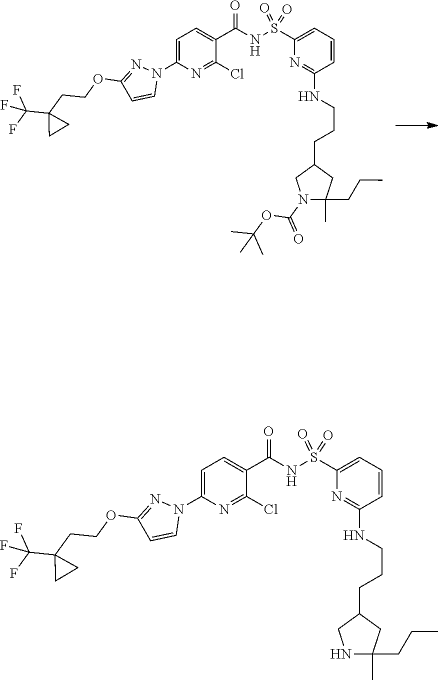 [SMILES: CCCC1(C)CC(CCCNc2cccc(S(=O)(=O)NC(=O)c3ccc(-n4ccc(OCCC5(C(F)(F)F)CC5)n4)nc3Cl)n2)CN1C(=O)OC(C)(C)C ; CCCC1(C)CC(CCCNc2cccc(S(=O)(=O)NC(=O)c3ccc(-n4ccc(OCCC5(C(F)(F)F)CC5)n4)nc3Cl)n2)CN1]

tert-Butyl 4-[3-[[6-[[2-chloro-6-[3-[2-[1-(trifluoromethyl)cyclopropyl]ethoxy]pyrazol-1-yl]pyridine-3-carbonyl]sulfamoyl]-2-pyridyl]amino]propyl]-2-methyl-2-propyl-pyrrolidine-1-carboxylate (533.3 mg, 0.6680 mmol) was dissolved in DCM (2.327 mL) and to the mixture was added TFA (2 mL, 26.73 mmol) and the mixture was stirred at room temperature for 2 h. Concentrated mixture to dryness under reduced pressure, added 1 mL of toluene and removed by rotary evaporation (45° C. water bath). Again added 1 mL of toluene and removed by rotary evaporation (45° C. water bath) then dried on the high vacuum giving 2-chloro-N-[[6-[3-(5-methyl-5-propyl-pyrrolidin-3-yl)propylamino]-2-pyridyl]sulfonyl]-6-[3-[2-[1-(trifluoromethyl)cyclopropyl]ethoxy]pyrazol-1-yl]pyridine-3-carboxamide (Trifluoroacetate salt) as a white, foamy solid. (542.6 mg, 100%). ESI-MS m/z calc. 697.2425, found 698.2 (M+1)⁺; Retention time: 0.63 minutes (LC method D).

### Step 11: 12-Methyl-12-propyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione

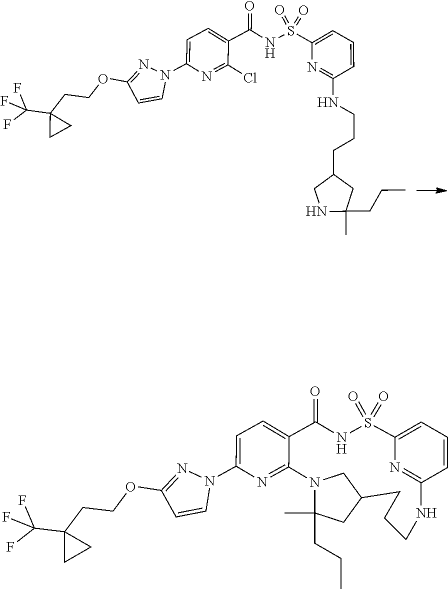 [SMILES: CCCC1(C)CC(CCCNc2cccc(S(=O)(=O)NC(=O)c3ccc(-n4ccc(OCCC5(C(F)(F)F)CC5)n4)nc3Cl)n2)CN1 ; CCCC1(C)CC2CCCNc3cccc(n3)S(=O)(=O)NC(=O)c3ccc(-n4ccc(OCCC5(C(F)(F)F)CC5)n4)nc3N1C2]

To a solution of 2-chloro-N-[[6-[3-(5-methyl-5-propyl-pyrrolidin-3-yl)propylamino]-2-pyridyl]sulfonyl]-6-[3-[2-[1-(trifluoromethyl)cyclopropyl]ethoxy]pyrazol-1-yl]pyridine-3-carboxamide (Trifluoroacetate salt) (542.6 mg, 0.6680 mmol) in NMP (32 mL) was added K₂CO₃ (646.4 mg, 4.677 mmol). The mixture was purged with N₂ for 5 min. The mixture was heated to 150° C. and stirred for 16 h. The mixture was cooled to room temperature and diluted with 1N HCl then extracted with EtOAc (2×) and the organic phase was then dried (sodium sulfate), filtered and concentrated to a brown oil which was dissolved in DMSO, filtered and purified using a reverse phase HPLC-MS method using a Luna C₁₈ (2) column (75×30 mm, 5 μm particle size) sold by Phenomenex (pn: 00C-4252-U0-AX), and a dual gradient run from 1-99% mobile phase B over 15.0 minutes (mobile phase A=H₂O (5 mM HCl), mobile phase B=acetonitrile, flow rate=50 mL/min, injection volume=950 μL and column temperature=25° C.) giving 12-methyl-12-propyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione as a white solid. (122 mg, 28%). ESI-MS m/z calc. 661.2658, found 662.2 (M+1)⁺; Retention time: 0.87 minutes (LC method Q).

### Step 12: 12-Methyl-12-propyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 1 (Compound 368), 12-methyl-12-propyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 2 (Compound 367), 12-methyl-12-propyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 3 (Compound 366), and 12-methyl-12-propyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 4 (Compound 365)

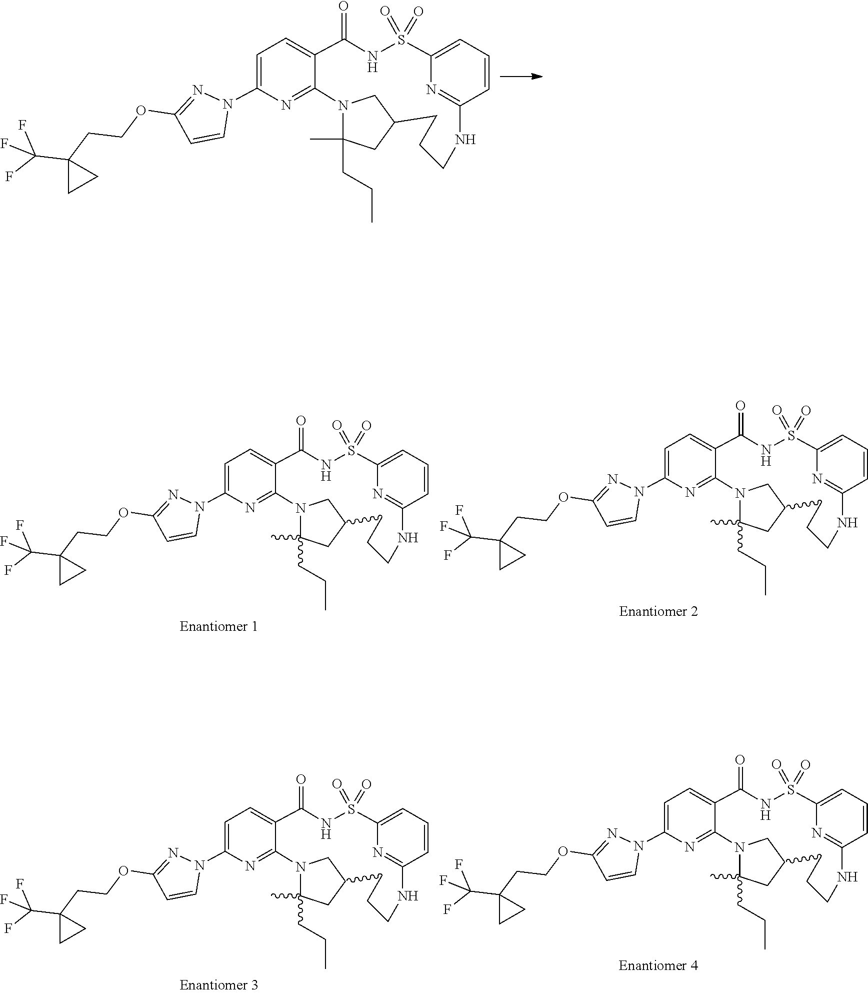

Subjected 12-methyl-12-propyl-8-(3-{2-[1-(trifluoromethyl)cyclopropyl] ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione (122 mg, 0.1844 mmol) to chiral separation by SFC chromatography using a ChiralPak IG (250×21.2 mm column, 5 μm particle size) with 40% MeOH/60% CO₂ mobile phase at 70 mL/min over 11.0 minutes (injection volume=500 μL of 32 mg/mL solution in MeOH giving four isomers:

First enantiomer to elute: 12-Methyl-12-propyl-8-(3-{2-[1-(trifluoromethyl) cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 1(29.2 mg, 96%). ¹H NMR (500 MHz, DMSO-d₆) δ 12.51 (s, 1H), 8.16 (d, J=2.8 Hz, 1H), 7.81 (d, J=8.1 Hz, 1H), 7.57 (t, J=7.8 Hz, 1H), 7.06 (d, J=7.1 Hz, 1H), 6.97 (s, 1H), 6.92 (d, J=8.1 Hz, 1H), 6.70 (d, J=8.5 Hz, 1H), 6.13 (d, J=2.8 Hz, 1H), 4.32 (t, J=7.1 Hz, 2H), 3.91 (s, 1H), 3.20 (s, 1H), 2.96 (d, J=13.2 Hz, 1H), 2.73 (s, 1H), 2.37 (s, 1H), 2.09 (t, J=7.3 Hz, 3H), 1.93-1.84 (m, 1H), 1.75 (s, 1H), 1.58 (s, 2H), 1.45 (s, 3H), 1.42-1.32 (m, 2H), 1.30 (dd, J=14.2, 10.3 Hz, 1H), 1.24 (s, 1H), 1.16 (d, J=8.2 Hz, 1H), 0.96 (d, J=4.9 Hz, 2H), 0.89 (s, 2H), 0.83 (t, J=7.2 Hz, 3H). ESI-MS calc. 661.2658, found 662.5 (M+1)⁺; Retention time: 2.37 minutes (LC method A).

Second enantiomer to elute, 12-Methyl-12-propyl-8-(3-[2-[1-(trifluoromethyl)cyclopropyl]ethoxy]-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 2 (11.1 mg, 36%). ¹H NMR (500 MHz, DMSO-d₆) δ 12.51 (s, 1H), 8.19 (d, J=2.9 Hz, 1H), 7.83 (d, J=8.2 Hz, 1H), 7.58 (t, J=7.8 Hz, 1H), 7.05 (d, J=7.0 Hz, 1H), 6.96 (s, 1H), 6.92 (d, J=7.9 Hz, 1H), 6.71 (d, J=8.5 Hz, 1H), 6.12 (t, J=3.4 Hz, 1H), 4.32 (t, J=7.0 Hz, 2H), 3.94 (s, 1H), 3.18 (s, 1H), 2.94 (d, J=13.2 Hz, 1H), 2.65 (s, 1H), 2.09 (t, J=7.5 Hz, 4H), 1.79 (d, J=15.7 Hz, 2H), 1.67 (d, J=11.6 Hz, 2H), 1.61 (d, J=4.6 Hz, 5H), 1.35 (d, J=9.2 Hz, 1H), 1.30 (d, J=17.7 Hz, 1H), 1.05 (s, 1H), 0.96 (d, J=5.0 Hz, 2H), 0.89 (s, 2H), 0.75 (t, J=7.1 Hz, 3H). ESI-MS calc. 661.2658, found 662.6 (M+1)⁺; Retention time: 2.36 minutes (LC method A).

Third enantiomer to elute, 12-Methyl-12-propyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 3(13.6 mg, 44%). ¹H NMR (500 MHz, DMSO-d₆) δ 12.51 (s, 1H), 8.19 (d, J=2.7 Hz, 1H), 7.83 (d, J=8.2 Hz, 1H), 7.58 (t, J=7.9 Hz, 1H), 7.05 (d, J=7.2 Hz, 1H), 6.97 (s, 1H), 6.92 (d, J=8.2 Hz, 1H), 6.71 (d, J=8.6 Hz, 1H), 6.12 (d, J=2.7 Hz, 1H), 4.32 (t, J=7.1 Hz, 2H), 3.98-3.88 (m, 1H), 3.18 (s, 1H), 2.94 (d, J=13.2 Hz, 1H), 2.64 (s, 1H), 2.09 (t, J=7.0 Hz, 4H), 1.79 (d, J=14.0 Hz, 2H), 1.67 (d, J=11.5 Hz, 2H), 1.61 (s, 3H), 1.59 (s, 1H), 1.36 (s, 1H), 1.34-1.27 (m, 2H), 1.10-1.01 (m, 1H), 0.96 (d, J=4.9 Hz, 2H), 0.88 (d, J=17.4 Hz, 2H), 0.75 (t, J=7.3 Hz, 3H). ESI-MS m/z calc. 661.2658, found 662.6 (M+1)⁺; Retention time: 2.36 minutes (LC method A).

Fourth and final enantiomer to elute, 12-Methyl-12-propyl-8-(3-{2-[1-(trifluoromethyl)cyclopropyl]ethoxy}-1H-pyrazol-1-yl)-2λ⁶-thia-3,9,11,18,23-pentaazatetracyclo[17.3.1.111,14.05,10]tetracosa-1(22),5,7,9,19(23),20-hexaene-2,2,4-trione, enantiomer 4(24.8 mg, 81%). ESI-MS m/z calc. 661.2658, found 662.6 (M+1)⁺; Retention time: 2.36 minutes (LC method A).

### Example 136: Preparation of (14S)-7-Benzyl-17-(4-tert-butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 1, (Compound 396) and (14S)-7-Benzyl-17-(4-tert-butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 2, (Compound 397)

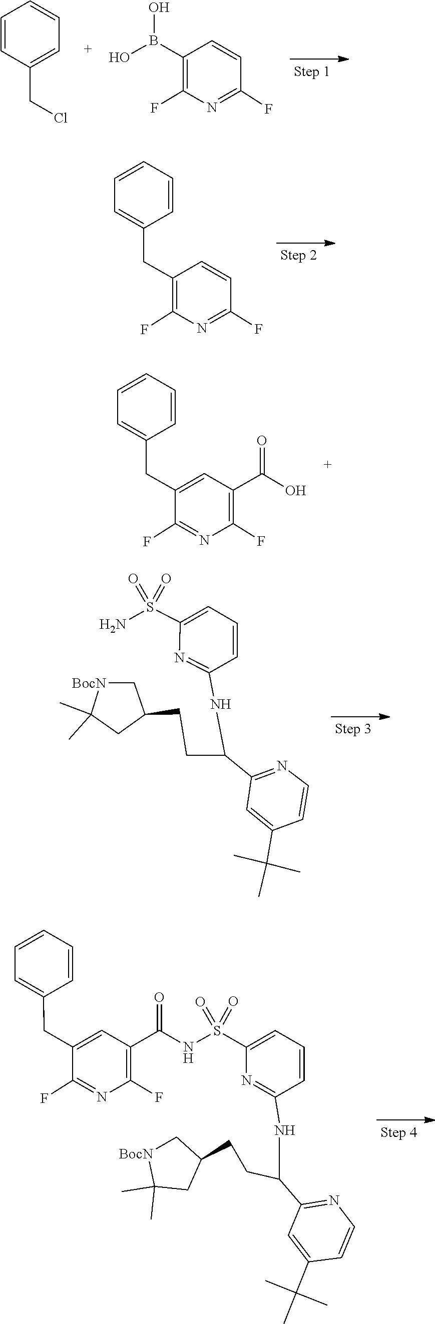

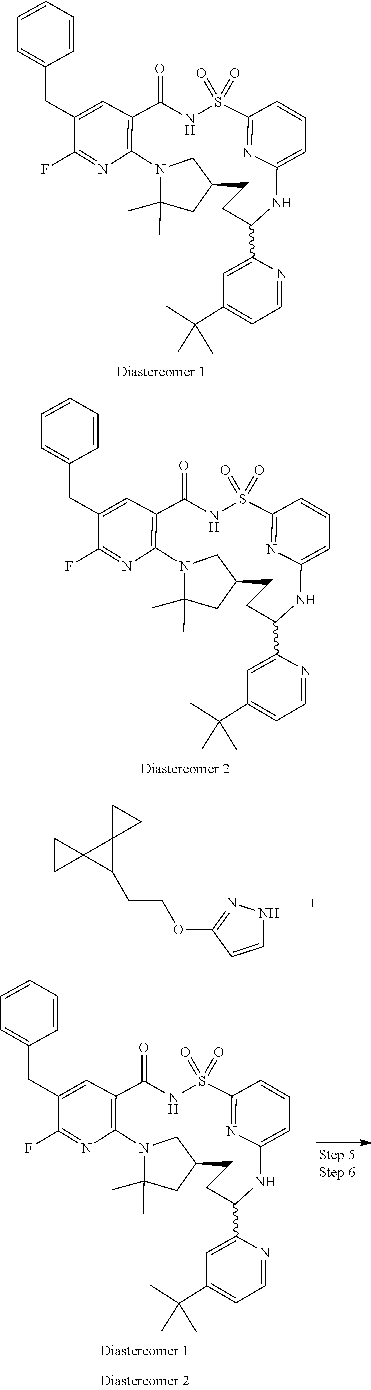 [SMILES: CC(C)(C)c1ccnc(C2CC[C@@H]3CN(c4nc(F)c(Cc5ccccc5)cc4C(=O)NS(=O)(=O)c4cccc(n4)N2)C(C)(C)C3)c1 ; CC(C)(C)c1ccnc(C2CC[C@@H]3CN(c4nc(F)c(Cc5ccccc5)cc4C(=O)NS(=O)(=O)c4cccc(n4)N2)C(C)(C)C3)c1 ; c1cc(OCCC2C3(CC3)C23CC3)n[nH]1 ; CC(C)(C)c1ccnc(C2CC[C@@H]3CN(c4nc(F)c(Cc5ccccc5)cc4C(=O)NS(=O)(=O)c4cccc(n4)N2)C(C)(C)C3)c1]

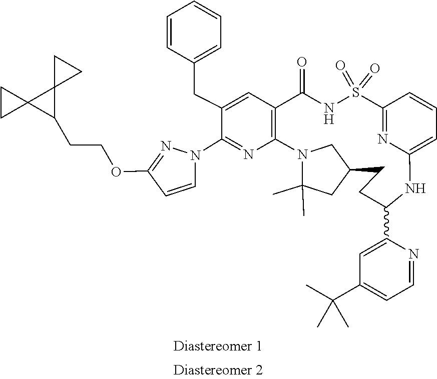 [SMILES: CC(C)(C)c1ccnc(C2CC[C@@H]3CN(c4nc(-n5ccc(OCCC6C7(CC7)C67CC7)n5)c(Cc5ccccc5)cc4C(=O)NS(=O)(=O)c4cccc(n4)N2)C(C)(C)C3)c1]

### Step 1: 3-Benzyl-2,6-difluoro-pyridine

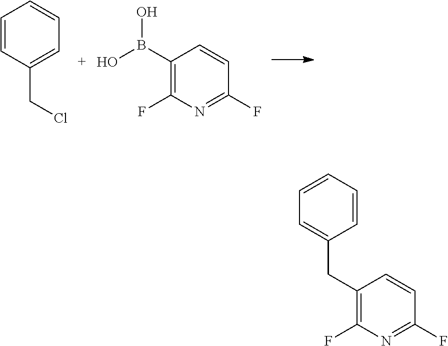 [SMILES: ClCc1ccccc1 ; OB(O)c1ccc(F)nc1F ; Fc1ccc(Cc2ccccc2)c(F)n1]

In a 250-mL round-bottomed flask, (2,6-difluoro-3-pyridyl)boronic acid (3.1351 g, 19.73 mmol), benzyl chloride (2.64 g, 20.86 mmol) and Pd(PPh₃)₄ (0.5525 g, 0.4781 mmol) were mixed with DME (80 mL) and aqueous Na₂CO₃ (40 mL of 2.0 M, 80.00 mmol). The resulting mixture was sparged with N₂ gas for 5 min, and then stirred under reflux at 110° C. for 92 h. It was cooled to room temperature and filtered through Celite (rinsed with 100 mL of EtOAc). The filtrate was diluted with H₂O (100 mL), and the layers were separated. The organic layer was kept aside, and the aqueous layer was extracted with EtOAc (100 mL). The combined organic extracts was washed with H₂O (100 mL) and saturated aqueous NaCl solution (100 mL), then dried over Na₂SO₄, filtered, and evaporated in vacuo. Purification by silica gel chromatography (220 g of silica) using a gradient eluent of 0 to 15% EtOAc in hexanes gave a colorless oil, 3-benzyl-2,6-difluoro-pyridine (2.6395 g, 65%). ¹H NMR (400 MHz, DMSO-d₆) δ 8.06 (dt, J=9.9, 8.0 Hz, 1H), 7.33-7.27 (m, 2H), 7.26-7.20 (m, 3H), 7.14 (dd, J=8.0, 2.7 Hz, 1H), 3.98 (s, 2H).

### Step 2: 5-Benzyl-2,6-difluoro-pyridine-3-carboxylic acid

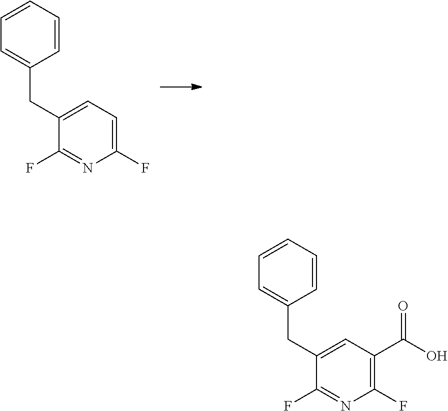 [SMILES: Fc1ccc(Cc2ccccc2)c(F)n1 ; O=C(O)c1cc(Cc2ccccc2)c(F)nc1F]

In a 100-mL round-bottomed flask, iPr₂NH (3.0 mL, 21.41 mmol) and THF (25 mL) were cooled to 78° C. under nitrogen atmosphere, upon which a hexanes solution of n-BuLi (8.0 mL of 2.5 M, 20.00 mmol) was added. The resulting mixture was stirred at 78° C. for 15 min, after which a THF (10 mL) solution of 3-benzyl-2,6-difluoro-pyridine (2.7675 g, 13.49 mmol) was added in one portion. The resulting mixture was stirred at 78° C. for 1 h, after which a stream of CO₂ (constant flow from a 16 gauge needle over 10 min) was bubbled into this mixture. The resulting mixture was stirred at 78° C. for 15 min, after which it was warmed to room temperature over 1 h. It was then poured into a flask containing aqueous HCl solution (1 N; 100 mL), and the mixture was extracted with EtOAc (3×75 mL). The combined organic extracts was washed with H₂O (100 mL) and saturated aqueous NaCl solution (100 mL), then dried over Na₂SO₄, filtered, and evaporated in vacuo to give a viscous yellow liquid that solidified under high vacuum overnight. The obtained product is probably only 50% pure: 5-benzyl-2,6-difluoro-pyridine-3-carboxylic acid (3.7 g, 55%).

### Step 3: tert-Butyl (4S)-4-[3-[[6-[(5-benzyl-2,6-difluoro-pyridine-3-carbonyl)sulfamoyl]-2-pyridyl]amino]-3-(4-tert-butyl-2-pyridyl)propyl]-2,2-dimethyl-pyrrolidine-1-carboxylate

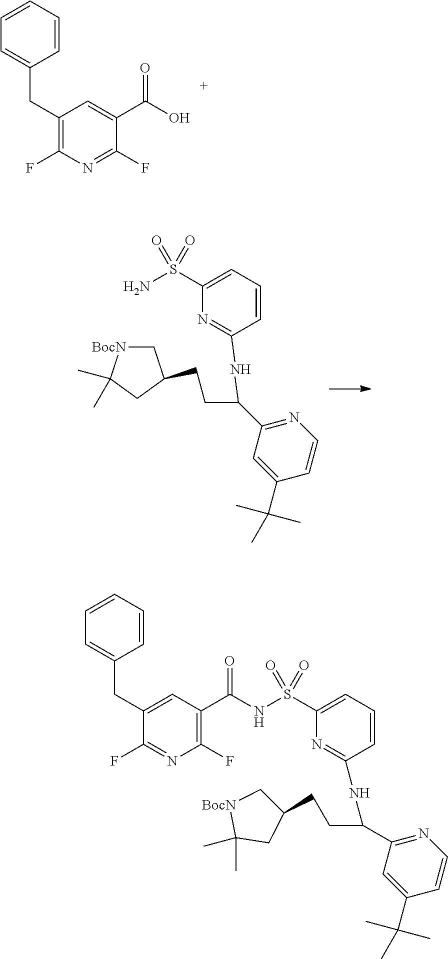 [SMILES: O=C(O)c1cc(Cc2ccccc2)c(F)nc1F ; CC(C)(C)OC(=O)N1C[C@@H](CCC(Nc2cccc(S(N)(=O)=O)n2)c2cc(C(C)(C)C)ccn2)CC1(C)C ; CC(C)(C)OC(=O)N1C[C@@H](CCC(Nc2cccc(S(=O)(=O)NC(=O)c3cc(Cc4ccccc4)c(F)nc3F)n2)c2cc(C(C)(C)C)ccn2)CC1(C)C]

In a 20-mL vial, 5-benzyl-2,6-difluoro-pyridine-3-carboxylic acid (546.7 mg, 1.097 mmol) was dissolved in THF (9.0 mL), to which CDI (400 mg, 2.467 mmol) was added. The resulting mixture was stirred at room temperature for 14 h. After this time, tert-butyl(4S)-4-[3-(4-tert-butyl-2-pyridyl)-3-[(6-sulfamoyl-2-pyridyl)amino]propyl]-2,2-dimethyl-pyrrolidine-1-carboxylate (900 mg, 1.649 mmol) and DBU (900 μL, 6.018 mmol) were added, and the resulting mixture was stirred at room temperature for 24 h. After this time, aqueous HCl solution (1 N, 9 mL) was added, and the mixture was extracted with EtOAc (3×3 mL). The combined organic extracts was washed with H₂O (7 mL) and saturated aqueous NaCl solution (7 mL), then dried over Na₂SO₄, filtered, and evaporated in vacuo to give an orange foam. Purification by silica gel chromatography (40 g of silica GOLD) using a gradient eluent of 0 to 90% EtOAc in hexanes gave recovered starting sulfonamide (380 mg, 40% recovered yield), as well as a mixture of diastereomers as the product: tert-butyl (4S)-4-[3-[[6-[(5-benzyl-2,6-difluoro-pyridine-3-carbonyl)sulfamoyl]-2-pyridyl]amino]-3-(4-tert-butyl-2-pyridyl)propyl]-2,2-dimethyl-pyrrolidine-1-carboxylate (82.5 mg, 6%). ESI-MS m/z calc. 776.35315, found 777.3 (M+1)⁺; Retention time: 1.89 minutes (LC method A).

### Step 4: (145)-7-Benzyl-17-(4-tert-butylpyridin-2-yl)-8-fluoro-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 1 and (14S)-7-benzyl-17-(4-tert-butylpyridin-2-yl)-8-fluoro-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, diastereomer 2

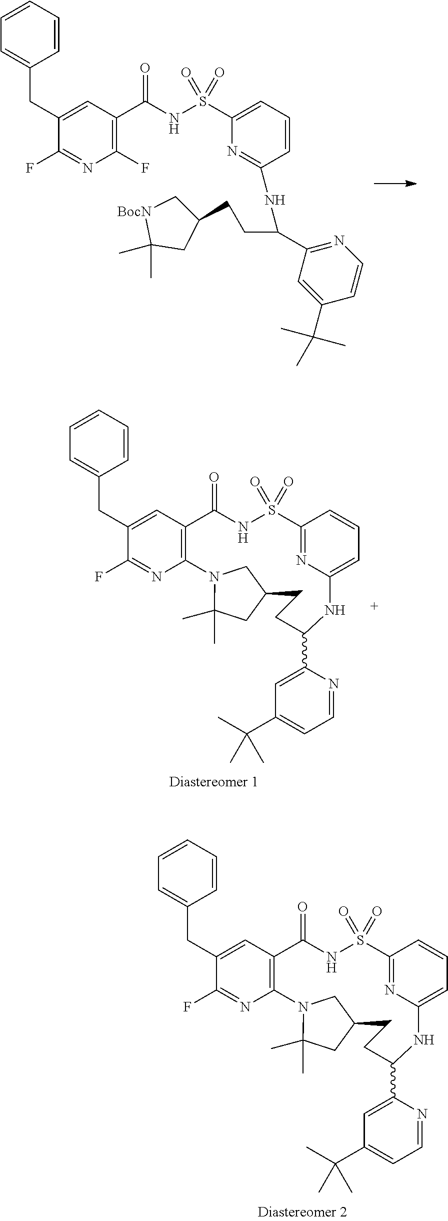 [SMILES: CC(C)(C)OC(=O)N1C[C@@H](CCC(Nc2cccc(S(=O)(=O)NC(=O)c3cc(Cc4ccccc4)c(F)nc3F)n2)c2cc(C(C)(C)C)ccn2)CC1(C)C ; CC(C)(C)c1ccnc(C2CC[C@@H]3CN(c4nc(F)c(Cc5ccccc5)cc4C(=O)NS(=O)(=O)c4cccc(n4)N2)C(C)(C)C3)c1 ; CC(C)(C)c1ccnc(C2CC[C@@H]3CN(c4nc(F)c(Cc5ccccc5)cc4C(=O)NS(=O)(=O)c4cccc(n4)N2)C(C)(C)C3)c1]

In a 1-dram vial, tert-butyl (4S)-4-[3-[[6-[(5-benzyl-2,6-difluoro-pyridine-3-carbonyl)sulfamoyl]-2-pyridyl]amino]-3-(4-tert-butyl-2-pyridyl)propyl]-2,2-dimethyl-pyrrolidine-1-carboxylate (80 mg, 0.08135 mmol) was dissolved in CH₂Cl₂ (1.0 mL). TFA (200 μL, 2.596 mmol) was added, and the resulting solution was allowed to stand at room temperature for 2 h. The mixture was then evaporated in vacuo, diluted with dioxane (1.0 mL), and evaporated in vacuo again. This gave a yellow oil, 100 mg (>100% yield).

In a 1-dram vial, the crude product from Step 1 was dissolved in NMP (1.0 mL), to which K₂CO₃ (150 mg, 1.085 mmol) was added. The resulting mixture was flushed with N₂, then stirred at 140° C. for 19 h. After cooling to room temperature, the reaction mixture was poured into 1 N HCl solution (5 mL) and extracted with EtOAc (3×3 mL). The combined organic extracts was washed with H₂O (5 mL) and saturated aqueous NaCl solution (5 mL), then dried over Na₂SO₄, filtered, and evaporated in vacuo. This material was then diluted with 1:1 DMSO:MeOH (2 mL), filtered, and purified by reverse-phase preparative chromatography using a C₁₈ column and a gradient eluent of 20 to 80% MeCN in H₂O containing 5 mM HCl solution to give 2 products:

Diastereomer 1, “Peak 1”: Earlier retention time on 30-min HPLC, (14S)-7-benzyl-17-(4-tert-butylpyridin-2-yl)-8-fluoro-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (12.1 mg, 23%). ¹H NMR (400 MHz, DMSO-d₆) δ 12.92-12.06 (bs, 1H), 8.71-8.56 (m, 1H), 8.16-7.85 (m, 2H), 7.84-7.57 (m, 3H), 7.30 (t, J=7.4 Hz, 3H), 7.24 (d, 7.0 Hz, 2H), 7.20 (t, J=7.0 Hz, 1H), 7.01-6.88 (m, 1H), 5.34-4.94 (m, 1H), 3.82 (s, 2H), 3.27-3.08 (m, 1H), 2.97-2.69 (m, 1H), 2.35-2.23 (m, 1H), 2.22-2.06 (m, 1H), 1.86 (dd, J=11.9, 5.6 Hz, 1H), 1.81-1.67 (m, 1H), 1.58-1.47 (m, 1H), 1.46 (s, 6H), 1.30 (s, 9H), 1.21-1.05 (m, 1H). [Note: 1H is missing from the overall count of 41 from the product (C36H41FN6O3S).]. ESI-MS m/z calc. 656.2945, found 657.3 (M+1)⁺; Retention time: 1.76 minutes (LC method A).

Diastereomer 2,“Peak 2”: Later retention time on 30-min HPLC, (14S)-7-benzyl-17-(4-tert-butylpyridin-2-yl)-8-fluoro-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (12.5 mg, 23%). ¹H NMR (400 MHz, DMSO-d₆) δ 13.28-11.78 (bs, 1H), 8.84-8.50 (m, 1H), 8.12-7.85 (m, 2H), 7.84 (d, J=10.0 Hz, 1H), 7.71 (t, J=7.8 Hz, 1H), 7.31 (t, J=7.5 Hz, 2H), 7.25 (d, J=7.0 Hz, 2H), 7.23-7.14 (m, 2H), 6.95 (d, J=8.1 Hz, 1H), 5.55-5.28 (m, 1H), 3.91-3.78 (m, 2H), 3.36-3.26 (m, 1H), 2.64 (t, J=10.5 Hz, 1H), 2.37-2.21 (m, 1H), 2.07-1.88 (m, 2H), 1.85 (dd, J=12.0, 5.1 Hz, 1H), 1.80-1.69 (m, 1H), 1.59 (s, 3H), 1.57-1.43 (m, 2H), 1.41 (s, 3H), 1.30 (s, 9H), 1.26-1.07 (m, 1H). ESI-MS m/z calc. 656.2945, found 657.3 (M+1)⁺; Retention time: 1.75 minutes (LC method A).

### Step 5: (14S)-7-Benzyl-17-(4-tert-butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaaza tetracyclo[17.3.1.111,14.05,10]tetracosa-1(23),5(10),6,8,19,21-hexaene-2,2,4-trione, diastereomer 1, (Compound 396)

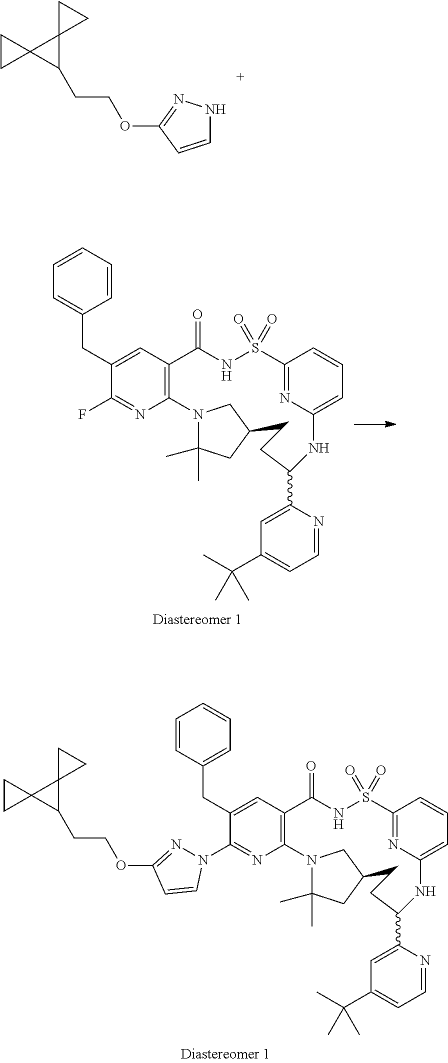 [SMILES: c1cc(OCCC2C3(CC3)C23CC3)n[nH]1 ; CC(C)(C)c1ccnc(C2CC[C@@H]3CN(c4nc(F)c(Cc5ccccc5)cc4C(=O)NS(=O)(=O)c4cccc(n4)N2)C(C)(C)C3)c1 ; CC(C)(C)c1ccnc(C2CC[C@@H]3CN(c4nc(-n5ccc(OCCC6C7(CC7)C67CC7)n5)c(Cc5ccccc5)cc4C(=O)NS(=O)(=O)c4cccc(n4)N2)C(C)(C)C3)c1]

In a 1-dram vial, a solution of (14S)-7-benzyl-17-(4-tert-butylpyridin-2-yl)-8-fluoro-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (12.1 mg, 0.01842 mmol) [Peak 1 from step 4] in DMSO-d₆ (400 μL) was mixed with 3-(2-dispiro[2.0.24.13]heptan-7-ylethoxy)-1H-pyrazole (12.0 mg, 0.05875 mmol), DABCO (5 mg, 0.04457 mmol), K₂CO₃ (20 mg, 0.1447 mmol) and DMSO (400 μL). The resulting mixture was stirred at room temperature for 12 h, then at 100° C. for 3 h, and then at 150° C. for 16 h. It was cooled to room temperature, and K₂CO₃ (20 mg, 0.1447 mmol) was added. The resulting mixture was stirred at 170° C. for 25 h. It was cooled to room temperature, then 1 N aqueous HCl solution (1.0 mL) was added, followed by EtOAc (1.0 mL). The phases were vigorously mixed and then allowed to settle into two layers. The organic layer was filtered and purified by reverse-phase preparative chromatography using a C₁₈ column and a gradient eluent of 1 to 99% acetonitrile in water containing 5 mM HCl solution to give two products (some racem/zation occurred during the reaction):
Diastereomer 1, “Peak 1”, earlier eluting compound on a 30-min HPLC run, (14S)-7-benzyl-17-(4-tert-butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (1.8 mg, 11%). ESI-MS m/z calc. 840.41455, found 841.5 (M+1)⁺; Retention time: 2.21 minutes (LC method A).
Diastereomer 2 “Peak 2”, later eluting compound on a 30-min HPLC run, (14S)-7-benzyl-17-(4-tert-butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Hydrochloride salt) (1.5 mg, 9%). ESI-MS m/z calc. 840.41455, found 841.4 (M+1)⁺; Retention time: 2.21 minutes (LC method A).

### Step 6: (14S)-7-Benzyl-17-(4-tert-butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaaza tetracyclo[17.3.1.111,14.05,10]tetracosa-1(23),5(10),6,8,19,21-hexaene-2,2,4-trione, diastereomer 2, (Compound 397)

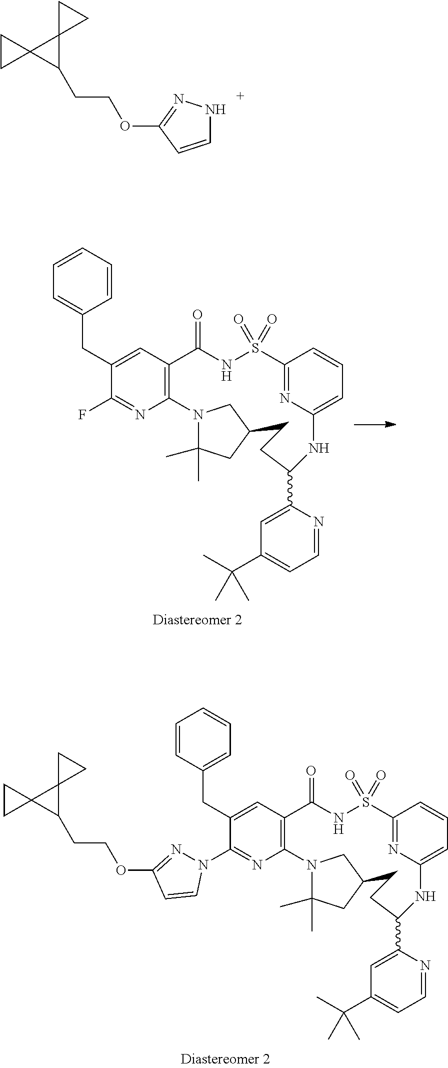 [SMILES: c1cc(OCCC2C3(CC3)C23CC3)n[nH]1 ; CC(C)(C)c1ccnc(C2CC[C@@H]3CN(c4nc(F)c(Cc5ccccc5)cc4C(=O)NS(=O)(=O)c4cccc(n4)N2)C(C)(C)C3)c1 ; CC(C)(C)c1ccnc(C2CC[C@@H]3CN(c4nc(-n5ccc(OCCC6C7(CC7)C67CC7)n5)c(Cc5ccccc5)cc4C(=O)NS(=O)(=O)c4cccc(n4)N2)C(C)(C)C3)c1]

In a 1-dram vial, a solution of (14S)-7-benzyl-17-(4-tert-butylpyridin-2-yl)-8-fluoro-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (12.5 mg, 0.01903 mmol) [Peak 2 from step 4] in DMSO-d₆ (400 μL) was mixed with 3-(2-dispiro[2.0.24.13]heptan-7-ylethoxy)-1H-pyrazole (12.2 mg, 0.05973 mmol), DABCO (5 mg, 0.04457 mmol), K₂CO₃ (20 mg, 0.1447 mmol) and DMSO (400 μL). The resulting mixture was stirred at room temperature for 12 h, then at 100° C. for 3 h, and then at 150° C. for 16 h. It was cooled to room temperature, and K₂CO₃ (20 mg, 0.1447 mmol) was added. The resulting mixture was stirred at 170° C. for 25 h. It was cooled to room temperature, then 1 N aqueous HCl solution (1.0 mL) was added, followed by EtOAc (1.0 mL). The phases were vigorously mixed and then allowed to settle into two layers. The organic layer was filtered and purified by reverse-phase preparative chromatography using a C₁₈ column and a gradient eluent of 1 to 99% acetonitrile in water containing 5 mM HCl solution to give two products (some racem/zation occurred during the reaction):
Diastereomer 1, “Peak 1”, earlier eluting compound on a 30-min HPLC run, (14S)-7-benzyl-17-(4-tert-butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Hydrochloride salt) (0.7 mg, 4%). ESI-MS m/z calc. 840.41455, found 841.5 (M+1)⁺; Retention time: 2.22 minutes (LC method A).

Diastereomer 2, “Peak 2”, later eluting compound on a 30-min HPLC run, (14S)-7-benzyl-17-(4-tert-butylpyridin-2-yl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Hydrochloride salt) (3.8 mg, 23%). ESI-MS m/z calc. 840.41455, found 841.5 (M+1)⁺; Retention time: 2.22 minutes ((LC method A).

### Example 137: Preparation of (14S)-7-Bromo-8-tert-butyl-17-(4-tert-butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 398)

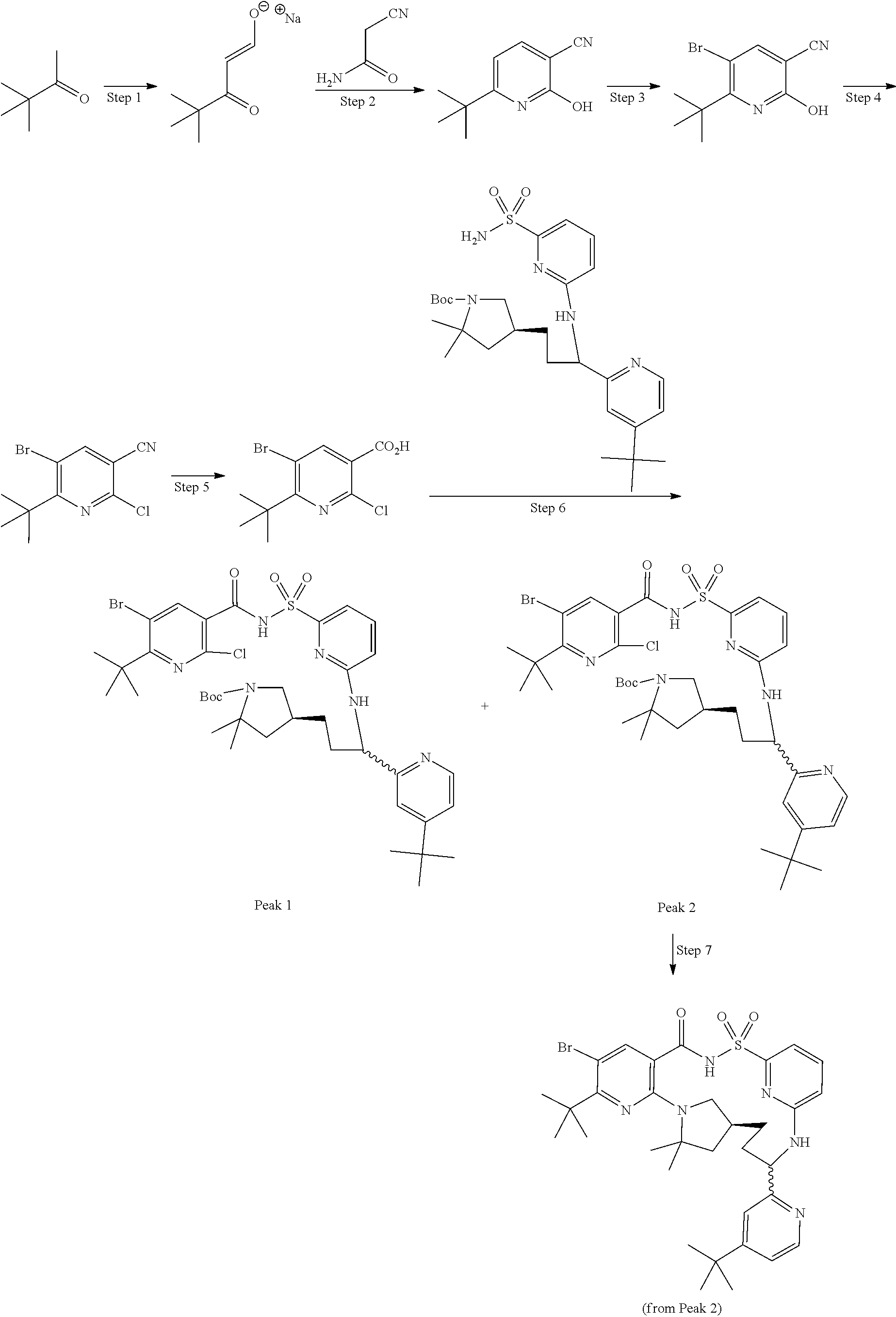

### Step 1: Sodium (1E)-4,4-dimethyl-3-oxopent-1-en-1-olate

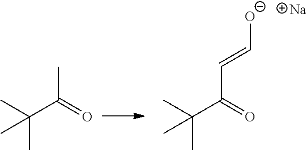

A mixture of 3,3-dimethylbutan-2-one (20.0 g, 200 mmol) and ethyl formate (16.1 mL, 200 mmol) was added slowly to sodium hydride (8.0 g, 200 mmol) in toluene (200 mL) and the reaction mixture was stirred overnight at room temperature. The solvent was evaporated under reduced pressure, and this was taken onto the next reaction without purification.

### Step 2: 6-tert-Butyl-2-hydroxypyridine-3-carbonitrile

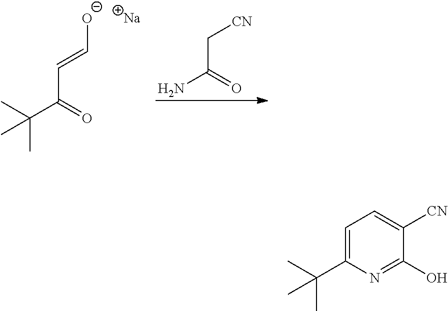

2-Cyanoacetamide (16.8 g, 200 mmol) was added to sodium (1E)-4,4-dimethyl-3-oxopent-1-en-1-olate (30.0 g, 200 mmol) in pyridine (100 mL) and the reaction mixture was refluxed overnight. The mixture was concentrated under reduced pressure. The residue was purified by silica gel chromatography (30% to 70% ethyl acetate/heptanes) to provide 6-tert-butyl-2-hydroxypyridine-3-carbonitrile (1.18 g, 19% yield over 2 steps) as a yellow solid. ¹H NMR (300 MHz, DMSO-d₆) δ 12.21 (br. s., 1H), 8.05 (d, J=7.5 Hz, 1H), 6.22 (d, J=7.6 Hz, 1H), 1.25 (s, 9H).

### Step 3: 5-Bromo-6-tert-butyl-2-hydroxypyridine-3-carbonitrile

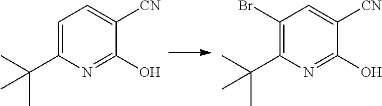

N-Bromosuccinimide (10.3 g, 57.9 mmol) was added to 6-tert-butyl-2-hydroxypyridine-3-carbonitrile (6.8 g, 38.6 mmol) in 1,2-dichloroethane (50 mL) and the reaction mixture was refluxed for 3 h. Water (50 mL) was added, and this mixture was extracted twice with DCM (2×50 mL). The organic phase was washed with water, dried with anhydrous sodium sulfate and concentrated under reduced pressure. The residue was purified by silica gel chromatography (0% to 10% MeOH/DCM) to provide 5-bromo-6-tert-butyl-2-hydroxypyridine-3-carbonitrile (6.4 g, 65%) as a white solid. ¹H NMR (300 MHz, DMSO-d₆) δ 8.36 (s, 1H), 1.43 (s, 9H).

### Step 4: 5-Bromo-6-tert-butyl-2-chloropyridine-3-carbonitrile

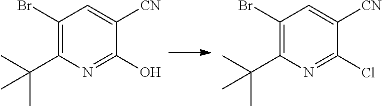

Phosphorus oxychloride (30.3 mL, 325.4 mmol) and phosphorus pentachloride (20.3 g, 97.6 mmol) were added to 5-bromo-6-tert-butyl-2-hydroxypyridine-3-carbonitrile (8.30 g, 32.5 mmol) and the reaction mixture was refluxed for 2 days. The reaction mixture was cooled to room temperature and concentrated under reduced pressure. Water (75 mL) was added to the residue, extracted ethyl acetate (3×75 mL), washed with brine (75 mL), dried with anhydrous sodium sulfate, filtered and concentrated under reduced pressure. The residue was purified by silica gel chromatography (0% to 50% ethyl acetate/heptanes) to provide 5-bromo-6-tert-butyl-2-chloroxypyridine-3-carbonitrile (3.5 g, 39%) as an off-white solid. ¹H NMR (300 MHz, CDCl₃) δ 8.08 (s, 1H), 1.52 (s, 9H).

### Step 5: 5-Bromo-6-tert-butyl-2-chloropyridine-3-carboxylic acid

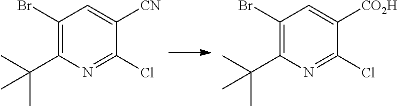

Potassium hydroxide (4.3 g, 76.8 mmol) in water (15 mL) was added to 5-bromo-6-tert-butyl-2-chloroxypyridine-3-carbonitrile in 2-propanol (15 mL), and the reaction mixture was heated at 90° C. overnight. Ethyl acetate (50 mL) was added and extracted with 1 N NaOH (3×50 mL). The aqueous phase was acidified to pH˜4 with 10% HCl, and the aqueous phase was extracted with ethyl acetate (3×75 mL), then washed with brine (100 mL). The combined organic extracts was dried with anhydrous sodium sulfate, filtered, concentrated under reduced pressure, and purified by reverse phase chromatography (0% to 100% methanol/water) to afford 5-bromo-6-tert-butyl-2-chloropyridine-3-carboxylic acid (2.3 g, 61% yield) as an off-white solid. ¹H NMR (300 MHz, CDCl₃) δ 8.46 (s, 1H), 1.53 (s, 9H).

### Step 6: tert-Butyl (4S)-4-[3-[[6-[(5-bromo-6-tert-butyl-2-chloro-pyridine-3-carbonyl)sulfamoyl]-2-pyridyl]amino]-3-(4-tert-butyl-2-pyridyl)propyl]-2,2-dimethyl-pyrrolidine-1-carboxylate; tert-butyl (4S)-4-[3-[[6-[(5-bromo-6-tert-butyl-2-chloro-pyridine-3-carbonyl)sulfamoyl]-2-pyridyl]amino]-3-(4-tert-butyl-2-pyridyl)propyl]-2,2-dimethyl-pyrrolidine-1-carboxylate

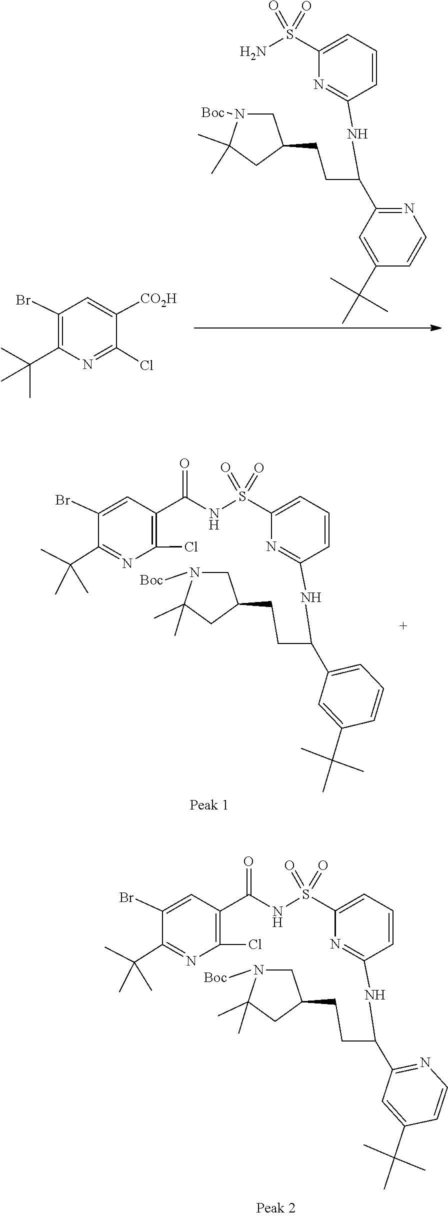 [SMILES: CC(C)(C)c1nc(Cl)c(C(=O)O)cc1Br ; CC(C)(C)OC(=O)N1C[C@@H](CCC(Nc2cccc(S(N)(=O)=O)n2)c2cc(C(C)(C)C)ccn2)CC1(C)C ; CC(C)(C)OC(=O)N1C[C@@H](CCC(Nc2cccc(S(=O)(=O)NC(=O)c3cc(Br)c(C(C)(C)C)nc3Cl)n2)c2cccc(C(C)(C)C)c2)CC1(C)C ; CC(C)(C)OC(=O)N1C[C@@H](CCC(Nc2cccc(S(=O)(=O)NC(=O)c3cc(Br)c(C(C)(C)C)nc3Cl)n2)c2cc(C(C)(C)C)ccn2)CC1(C)C]

In a 100-mL round-bottomed flask, 5-bromo-6-tert-butyl-2-chloro-pyridine-3-carboxylic acid (2.6298 g, 8.989 mmol) was mixed with DCM (30 mL) and cooled to 0° C., to which DMF (100 μL, 1.291 mmol) and oxalyl chloride (2.0 mL, 22.93 mmol) were added. The resulting solution was warmed to room temperature over 2 h. Upon verifying that the acyl chloride has indeed formed [with a small sample quench using morpholine to give (5-bromo-6-tert-butyl-2-chloro-3-pyridyl)-morpholino-methanone], the mixture was evaporated in vacuo.

Separately, in a 250-mL round-bottomed flask, a solution of tert-butyl (4S)-4-[3-(4-tert-butyl-2-pyridyl)-3-[(6-sulfamoyl-2-pyridyl)amino]propyl]-2,2-dimethyl-pyrrolidine-1-carboxylate (4.0082 g, 7.345 mmol) and DIPEA (10 mL, 57.41 mmol) in DCM (60 mL) was prepared. The crude acyl chloride generated above was dissolved in DCM (20 mL) and added to the sulfonamide solution. The resulting mixture was stirred at room temperature for 72 h. It was then poured into 0.5 N aqueous HCl solution (250 mL) and extracted with EtOAc (3×100 mL). The combined organic extracts were washed with H₂O (200 mL) and brine (200 mL), then dried over Na₂SO₄, filtered, and evaporated in vacuo. Purification by silica gel chromatography using a gradient eluent of 0 to 80% EtOAc in hexanes gave two products with the same mass on UPLC:

“Peak 1”, later-eluting product on silica gel, slightly earlier retention time on reverse-phase UPLC, orange foam, tert-butyl (4S)-4-[3-[[6-[(5-bromo-6-tert-butyl-2-chloro-pyridine-3-carbonyl)sulfamoyl]-2-pyridyl]amino]-3-(4-tert-butyl-2-pyridyl)propyl]-2,2-dimethyl-pyrrolidine-1-carboxylate (891.5 mg, 14%); ESI-MS m/z calc. 818.25916, found 819.2 (M+1)⁺; Retention time: 2.03 minutes (LC method A).

“Peak 2”, earlier-eluting product on silica gel, slightly later retention time on reverse-phase UPLC, orange foam, tert-butyl (4S)-4-[3-[[6-[(5-bromo-6-tert-butyl-2-chloro-pyridine-3-carbonyl)sulfamoyl]-2-pyridyl]amino]-3-(4-tert-butyl-2-pyridyl)propyl]-2,2-dimethyl-pyrrolidine-1-carboxylate (1.8010 g, 21%); ESI-MS m/z calc. 818.25916, found 819.2 (M+1)⁺; Retention time: 2.04 minutes (LC method A).

### Step 7: (14S)-7-Bromo-8-tert-butyl-17-(4-tert-butylpyridin-2-yl)-12,12-dimethyl-R⁶-thia-3,9,11,18,23-pentaazatetracyclo[17.3.1.111,14.05,10]tetracosa-1(23),5(10),6,8,19,21-hexaene-2,2,4-trione (Compound 398)

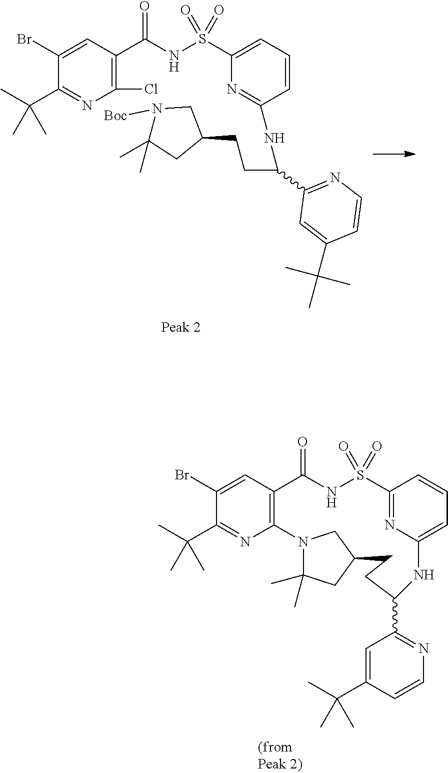 [SMILES: CC(C)(C)OC(=O)N1C[C@@H](CCC(Nc2cccc(S(=O)(=O)NC(=O)c3cc(Br)c(C(C)(C)C)nc3Cl)n2)c2cc(C(C)(C)C)ccn2)CC1(C)C ; CC(C)(C)c1ccnc(C2CC[C@@H]3CN(c4nc(C(C)(C)C)c(Br)cc4C(=O)NS(=O)(=O)c4cccc(n4)N2)C(C)(C)C3)c1]

Stage 1: In a 20-mL vial, tert-butyl (4S)-4-[3-[[6-[(5-bromo-6-tert-butyl-2-chloro-pyridine-3-carbonyl)sulfamoyl]-2-pyridyl]amino]-3-(4-tert-butyl-2-pyridyl)propyl]-2,2-dimethyl-pyrrolidine-1-carboxylate (1.8010 g, 1.537 mmol) was dissolved in DCM (8.0 mL), to which TFA (2 mL, 25.96 mmol) was added. The resulting solution was stirred at room temperature for 21 h. It was then evaporated in vacuo to give crude solid.

Stage 2: In a 100-mL round-bottomed flask, the product from Stage 1 was dissolved in NMP (25 mL), to which K₂CO₃ (2.723 g, 19.70 mmol) was added. A reflux condenser was mounted above the flask, and the mixture was stirred at 140° C. for 18 h, then at 160° C. for 23 h. It was then cooled to room temperature and poured into 1 N HCl (100 mL). The mixture was extracted with EtOAc (3×100 mL). The combined organic extracts were washed with H₂O (200 mL) and brine (200 mL), then dried over Na₂SO₄, filtered, and evaporated in vacuo. Purification by silica gel chromatography using a gradient eluent of 0 to 90% EtOAc in hexanes gave a beige solid, (14S)-7-bromo-8-tert-butyl-17-(4-tert-butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (621.3 mg, 56%); ¹H NMR (400 MHz, DMSO-d₆) δ 8.40 (d, J=5.2 Hz, 1H), 7.77 (s, 1H), 7.72-7.57 (m, 2H), 7.45 (d, J=1.9 Hz, 1H), 7.23 (dd, J=5.3, 1.9 Hz, 1H), 7.09 (d, J=7.2 Hz, 1H), 6.86 (d, J=8.4 Hz, 1H), 5.23 (ddd, J=12.3, 9.3, 3.2 Hz, 1H), 3.20 (dd, J=10.5, 6.8 Hz, 1H), 2.75 (t, J=10.6 Hz, 1H), 2.29-2.15 (m, 1H), 1.97-1.81 (m, 3H), 1.76-1.69 (m, 1H), 1.67-1.57 (m, 1H), 1.57 (s, 3H), 1.51 (s, 3H), 1.46 (s, 9H), 1.23 (s, 9H). [Note: 2H is missing from the overall count of 43 from the product (C₃₃H₄₃BrN₆O₃S).] ESI-MS m/z calc. 682.2301, found 683.2 (M+1)⁺; Retention time: 1.83 minutes (LC method A).

### Example 138: Preparation of (14S)-8-tert-Butyl-17-(4-tert-butylpyridin-2-yl)-12,12-dimethyl-7-[(1E)-2-phenylethenyl]-2λ⁶-thia-3,9,11,18,23-pentaazatetracyclo[17.3.1.111,14.05,10]tetracosa-1(23),5(10),6,8,19,21-hexaene-2,2,4-trione (Compound 399)

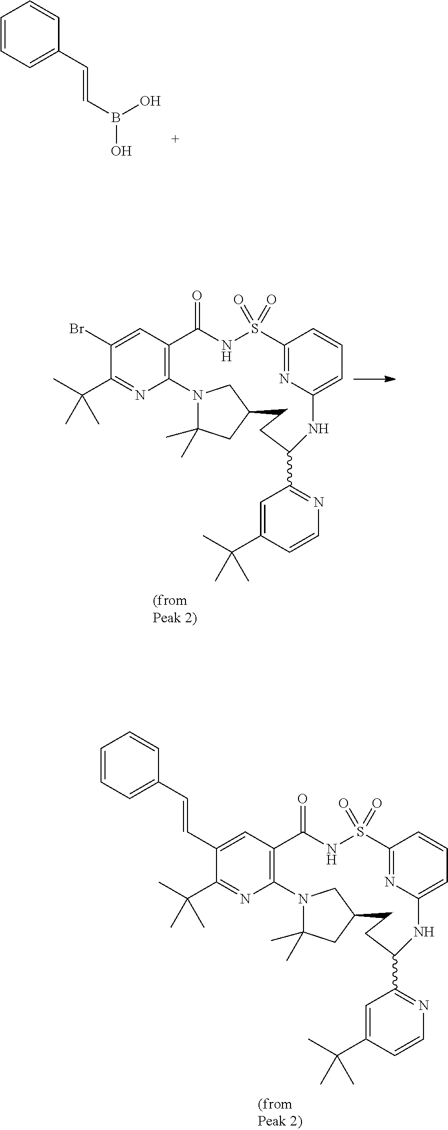 [SMILES: OB(O)/C=C/c1ccccc1 ; CC(C)(C)c1ccnc(C2CC[C@@H]3CN(c4nc(C(C)(C)C)c(Br)cc4C(=O)NS(=O)(=O)c4cccc(n4)N2)C(C)(C)C3)c1 ; CC(C)(C)c1ccnc(C2CC[C@@H]3CN(c4nc(C(C)(C)C)c(/C=C/c5ccccc5)cc4C(=O)NS(=O)(=O)c4cccc(n4)N2)C(C)(C)C3)c1]

In a 1-dram vial, [(E)-styryl]boronic acid (41.2 mg, 0.2784 mmol), (14S)-7-bromo-8-tert-butyl-17-(4-tert-butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (102.5 mg, 0.1424 mmol; originating from Peak 2), Pd(PPh₃)₄ (16.3 mg, 0.01411 mmol) and aqueous Na₂CO₃ (400 μL of 2.0 M, 0.8000 mmol) were mixed together with dioxane (600 μL). The resulting mixture was sparged under nitrogen under sonication for 10 min. A pressure-relief cap was fitted onto the vial, and the reaction mixture was stirred vigorously at 110° C. for 18 h. It was cooled to room temperature, then 1 N HCl (1.0 mL) was added, followed by EtOAc (1.0 mL). The phases were vigorously mixed and then allowed to settle into two layers. The organic layer was filtered and purified by reverse-phase preparative chromatography using a C₁₈ column and a gradient eluent of 1 to 99% acetonitrile in water containing 5 mM HCl solution to give (14S)-8-tert-butyl-17-(4-tert-butylpyridin-2-yl)-12,12-dimethyl-7-[(1E)-2-phenylethenyl]-2λ⁶-thia-3,9,11,18,23-pentaazatetracyclo[17.3.1.111,14.05,10]tetracosa-1(23),5(10),6,8,19,21-hexaene-2,2,4-trione (53.3 mg, 53%); ¹H NMR (400 MHz, DMSO-d₆) δ 12.88-12.21 (bs, 1H), 8.56 (s, 1H), 7.87 (s, 1H), 7.86-7.72 (m, 1H), 7.70 (t, J=7.9 Hz, 1H), 7.62-7.50 (m, 3H), 7.39 (t, J=7.6 Hz, 2H), 7.27 (t, J=7.3 Hz, 1H), 7.19 (d, J=7.2 Hz, 1H), 6.94 (d, J=8.5 Hz, 1H), 6.83 (d, J=15.9 Hz, 1H), 5.47-5.28 (m, 1H), 3.31 (dd, J=10.2, 6.6 Hz, 2H), 2.74 (t, J=10.5 Hz, 1H), 2.36-2.20 (m, 1H), 2.05-1.90 (m, 2H), 1.86 (dd, J=11.7, 5.2 Hz, 1H), 1.81-1.72 (m, 1H), 1.64 (s, 3H), 1.63-1.56 (m, 1H), 1.55 (s, 3H), 1.44 (s, 9H), 1.29 (s, 9H). [Note: 2H is missing from the overall count of 50 from the product (C₄₁H₅₀N₆O₃S).] ESI-MS m/z calc. 706.3665, found 707.7 (M+1)⁺; Retention time: 2.0 minutes (LC method A).

### Example 139: Preparation of (14S)-8-tert-Butyl-17-(4-tert-butylpyridin-2-yl)-12,12-dimethyl-7-(2-phenylethyl)-2λ⁶-thia-3,9,11,18,23-pentaazatetracyclo [17.3.1.111,14.05,10]tetracosa-1(23),5(10),6,8,19,21-hexaene-2,2,4-trione (Compound 400)

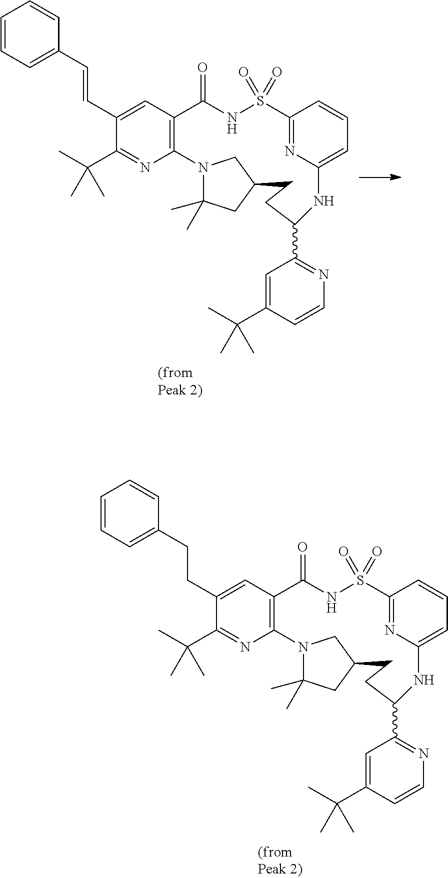 [SMILES: CC(C)(C)c1ccnc(C2CC[C@@H]3CN(c4nc(C(C)(C)C)c(/C=C/c5ccccc5)cc4C(=O)NS(=O)(=O)c4cccc(n4)N2)C(C)(C)C3)c1 ; CC(C)(C)c1ccnc(C2CC[C@@H]3CN(c4nc(C(C)(C)C)c(CCc5ccccc5)cc4C(=O)NS(=O)(=O)c4cccc(n4)N2)C(C)(C)C3)c1]

In a 5-mL microwave vial, (14S)-8-tert-butyl-17-(4-tert-butylpyridin-2-yl)-12,12-dimethyl-7-[(1E)-2-phenylethenyl]-2λ⁶-thia-3,9,11,18,23-pentaazatetracyclo[17.3.1.111,14.05,10]tetracosa-1(23),5(10),6,8,19,21-hexaene-2,2,4-trione (18.2 mg, 0.02574 mmol; originating from Peak 2) was suspended in EtOH (1.5 mL), and this mixture was sparged with nitrogen over 5 min. Pd(OH)₂/C (4.6 mg of 10% w/w, 0.003276 mmol) was added, and the resulting mixture was stirred at 50° C. under a balloon of H₂ (2 L, 79.37 mmol) for 16 h. It was then cooled to room temperature and filtered over Celite (MeOH rinse). The obtained liquid was evaporated in vacuo, and re-subjected to the reaction conditions: H₂ (2 L, 79.37 mmol), Pd(OH)₂/C (4.6 mg of 10% w/w, 0.003276 mmol) and EtOH (1.5 mL) at 50° C. for 16 h. It was then cooled to room temperature and filtered over Celite (MeOH rinse). The obtained liquid was evaporated in vacuo, re-dissolved in MeOH (1 mL), filtered, and purified by reverse-phase preparative chromatography using a C₁₈ column and a gradient eluent of 1 to 99% MeCN in H₂O containing 5 mM HCl solution to give a white solid, (14S)-8-tert-butyl-17-(4-tert-butylpyridin-2-yl)-12,12-dimethyl-7-(2-phenylethyl)-2λ⁶-thia-3,9,11,18,23-pentaazatetracyclo[17.3.1.111,14.05,10]tetracosa-1(23),5(10),6,8,19,21-hexaene-2,2,4-trione (13.2 mg, 72%); ¹H NMR (400 MHz, DMSO-d₆) δ 12.67-12.28 (bs, 1H), 8.74 (d, J=6.1 Hz, 1H), 8.24-8.00 (m, 2H), 7.97-7.87 (m, 1H), 7.75 (dd, J=8.5, 7.2 Hz, 1H), 7.69 (s, 1H), 7.34 (s, 2H), 7.33 (s, 2H), 7.25 (s, 1H), 7.25-7.17 (m, 1H), 7.00 (d, J=8.5 Hz, 1H), 5.58-5.47 (m, 1H), 3.41-3.27 (m, 1H), 3.07-2.83 (m, 3H), 2.77-2.60 (m, 2H), 2.42-2.31 (m, 1H), 2.06-1.93 (m, 2H), 1.84 (dd, J=11.7, 5.2 Hz, 1H), 1.80-1.72 (m, 1H), 1.64 (s, 3H), 1.61-1.52 (m, 1H), 1.52 (s, 3H), 1.52-1.44 (m, 1H), 1.38 (s, 9H), 1.34 (s, 9H). ESI-MS m/z calc. 708.38214, found 709.3 (M+1)⁺; Retention time: 2.01 minutes (LC method A).

### Example 140: Preparation of (14S)-8-tert-Butyl-17-(4-tert-butylpyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (Compound 401)

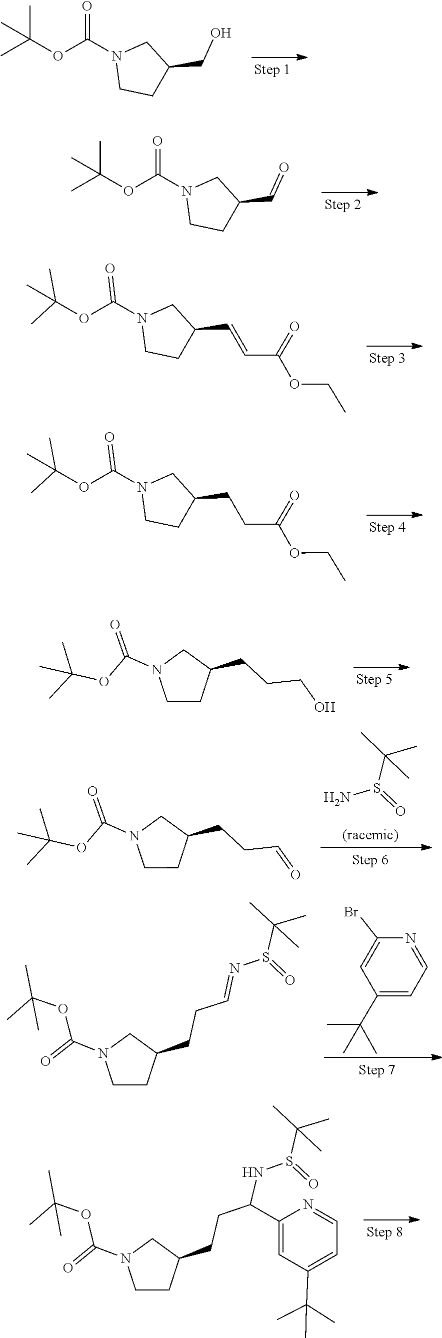

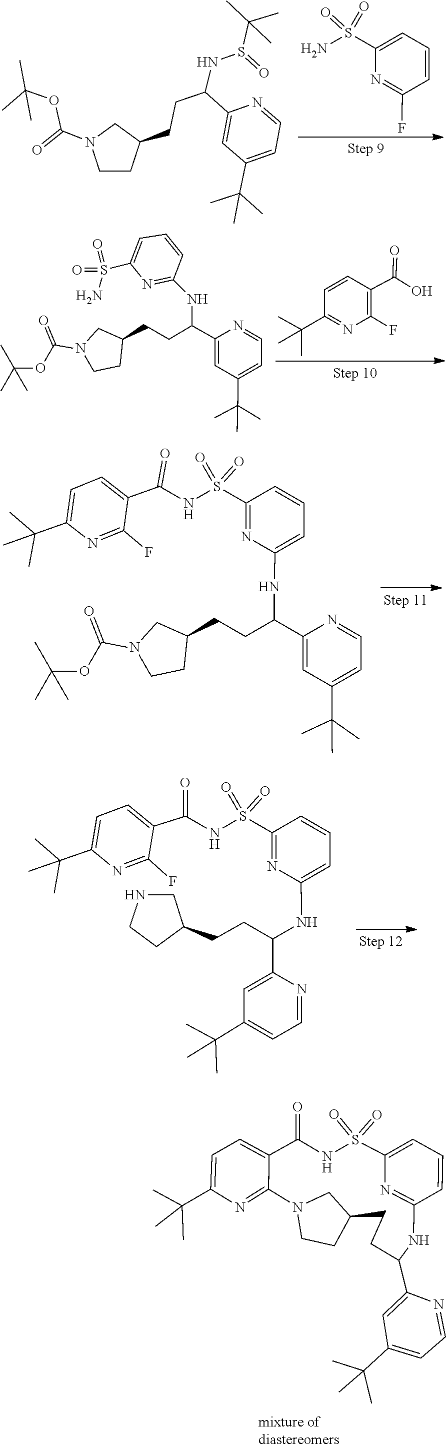

### Step 1: tert-Butyl (3S)-3-formylpyrrolidine-1-carboxylate

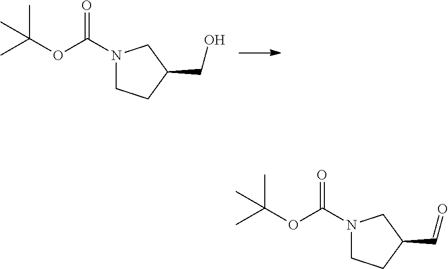

A solution of tert-butyl (3S)-3-(hydroxymethyl)pyrrolidine-1-carboxylate (25 g, 124.22 mmol) in dichloromethane (200 mL) was cooled to 0° C. A solution of sodium bromide (1.3 g, 12.634 mmol) and sodium bicarbonate (1.5 g, 17.856 mmol) dissolved in water (50 mL) was added. After 15 min of stirring at 0° C., TEMPO (200 mg, 1.2800 mmol) was added, followed by a slow addition of sodium hypochlorite in water (130 mL of 1.1 M, 143.00 mmol) dropwise keeping the internal temperature in the 6-8° C. range. The organic layer was separated and the aqueous layer was extracted with DCM (200 mL). The combined organic layers were washed with brine (200 mL), dried over Na₂SO₄, filtered, and concentrated to yield crude tert-butyl (3S)-3-formylpyrrolidine-1-carboxylate (22.15 g, 85%) as a yellow oil. ¹H NMR (400 MHz, CDCl₃) δ 9.70 (d, J=1.5 Hz, 1H), 3.78-3.62 (m, 1H), 3.58-3.47 (m, 1H), 3.42-3.30 (m, 2H), 3.13-3.03 (m, 1H), 2.28-2.00 (m, 2H), 1.47 (s, 9H).

### Step 2: tert-Butyl (3R)-3-[(E)-3-ethoxy-3-oxo-prop-1-enyl]pyrrolidine-1-carboxylate

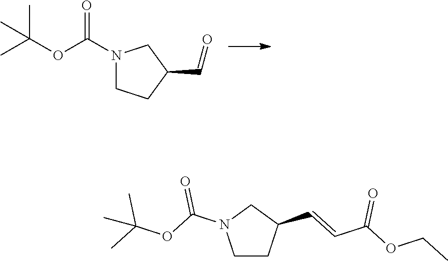 [SMILES: CC(C)(C)OC(=O)N1CC[C@H](C=O)C1 ; CCOC(=O)/C=C/[C@H]1CCN(C(=O)OC(C)(C)C)C1]

A flame-dried round-bottomed flask was charged with ethyl 2-diethoxyphosphorylacetate (27 g, 120.43 mmol) and THF (350 mL). The solution was cooled to 78° C. and then a solution of NaHMDS in THF (110 mL of 1 M, 110.00 mmol) was added. The mixture was stirred at −78° C. for 30 min, and then a solution of tert-butyl (3S)-3-formylpyrrolidine-1-carboxylate (22.15 g, 105.61 mmol) in THF (100 mL) was added dropwise. The mixture was stirred at −78° C. for 15 min. The reaction was quenched through the addition of a saturated aqueous solution of NH₄Cl (200 mL), the layers were separated and the aqueous phase was extracted EtOAc (3×150 mL). The combined organic phases were washed with brine (200 mL) and dried on anhydrous sodium sulfate, filtered and concentrated under reduced pressure. The crude material was purified through silica gel column chromatography using a gradient of 0 to 50% EtOAc in heptanes to afford tert-butyl (3R)-3-[(E)-3-ethoxy-3-oxo-prop-1-enyl]pyrrolidine-1-carboxylate (22.3 g, 78%) as a clear oil. ¹H NMR (400 MHz, CDCl₃) δ 6.89 (dd, J=15.7, 7.8 Hz, 1H), 5.88 (d, J=15.6 Hz, 1H), 4.20 (q, J=7.1 Hz, 2H), 3.66-3.41 (m, 2H), 3.40-3.26 (m, 1H), 3.21-3.08 (m, 1H), 3.21-3.08 (m, 1H), 3.01-2.86 (m, 1H), 2.13-2.02 (m, 1H), 1.85-1.72 (m, 1H), 1.46 (s, 9H), 1.29 (t, J=7.2 Hz, 3H). ESI-MS m/z calc. 269.1627, found 214.2 (M-tBu+2H=M−55)⁺; Retention time: 1.88 minutes (LC method E).

### Step 3: tert-Butyl (3S)-3-(3-ethoxy-3-oxo-propyl)pyrrolidine-1-carboxylate

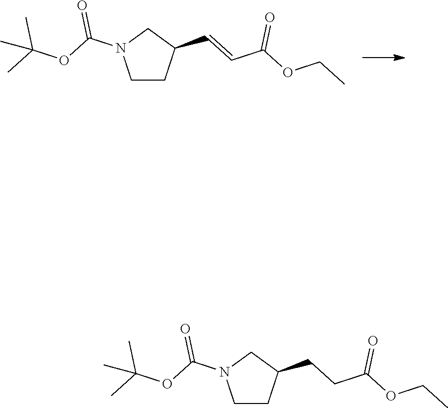

10% Palladium on carbon (50% wet, 5% w/w, 2.33 g, 1.0947 mmol) was added to a mixture of tert-butyl(3R)-3-[(E)-3-ethoxy-3-oxo-prop-1-enyl]pyrrolidine-1-carboxylate (22.3 g, 82.796 mmol) in methanol (150 mL). Hydrogen was bubbled into the suspension for 2 min, and then the reaction mixture was stirred under hydrogen atmosphere for 20 h. The reaction mixture was filtered through Celite and rinsed with methanol. The filtrate was concentrated in vacuo to give crude tert-butyl (3S)-3-(3-ethoxy-3-oxo-propyl)pyrrolidine-1-carboxylate (21.7 g, 96%) as a colorless oil. ¹H NMR (400 MHz, CDCl₃) δ 4.19-4.07 (m, 2H), 3.61-3.36 (m, 2H), 3.42-3.39 (m, 1H), 2.97-2.78 (m, 1H), 2.39-2.27 (m, 2H), 2.15-2.10 (m, 1H), 2.04-1.92 (m, 1H), 1.75-1.68 (m, 2H), 1.55-1.36 (m, 10H), 1.30-1.18 (m, 3H). ESI-MS m/z calc. 271.1784, found 216.2 (M-tBu+2H=M−55)⁺; Retention time: 1.88 minutes (LC method E).

### Step 4: tert-Butyl (3S)-3-(3-hydroxypropyl)pyrrolidine-1-carboxylate

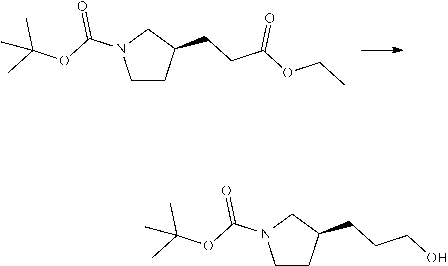

To a solution of tert-butyl (3S)-3-(3-ethoxy-3-oxo-propyl)pyrrolidine-1-carboxylate (13.8 g, 49.331 mmol) in THF (125 mL) at 0° C. was added a solution of LiBH₄ in THF (67 mL of 2 M, 134.00 mmol). The reaction mixture was stirred at room temperature for 16 h. The reaction mixture was added slowly to a saturated aqueous solution of NH₄Cl (200 mL) at 0° C. The product was extracted with EtOAc (3×200 mL). The combined organic layers were washed with brine (200 mL), dried over anhydrous sodium sulfate, filtered and concentrated under reduced pressure. The crude material was purified through silica gel column chromatography using a gradient of 20 to 100% EtOAc in heptanes to afford tert-butyl (3S)-3-(3-hydroxypropyl)pyrrolidine-1-carboxylate (11 g, 91%) as a clear oil. Isolated product has 91.8% enantiomeric ratio. ¹H NMR (400 MHz, CDCl₃) δ 3.66 (t, J=6.5 Hz, 2H), 3.61-3.36 (m, 2H), 3.31-3.17 (m, 1H), 2.93-2.80 (m, 1H), 2.19-2.07 (m, 1H), 2.03-1.96 (m, 1H), 1.76-1.54 (m, 3H), 1.54-1.38 (m, 12H). ESI-MS m/z calc. 229.1678, found 252.2 (M+Na=M+23)⁺; 174.2 (M-tBu+2H=M−55)⁺; Retention time: 3.32 minutes (LC method F).

### Step 5: tert-Butyl (3S)-3-(3-oxopropyl)pyrrolidine-1-carboxylate

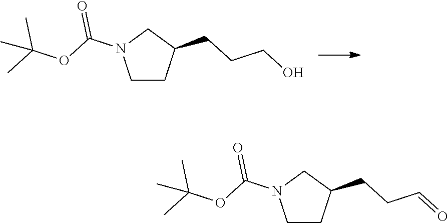 [SMILES: CC(C)(C)OC(=O)N1CC[C@H](CCCO)C1 ; CC(C)(C)OC(=O)N1CC[C@H](CCC=O)C1]

A solution of tert-butyl (3S)-3-(3-hydroxypropyl)pyrrolidine-1-carboxylate (2 g, 7.8494 mmol) in dichloromethane (20 mL) was cooled to 0° C. A solution of sodium bromide (135 mg, 1.3120 mmol) and sodium bicarbonate (150 mg, 1.7856 mmol) dissolved in water (5 mL) was added. After 15 min of stirring at 0° C., TEMPO (45 mg, 0.2880 mmol) was added, followed by a slow addition of sodium hypochlorite in water (9.6 mL of 1.1 M, 10.560 mmol) dropwise keeping the internal temperature in the 6-8° C. range. The organic layer was separated and the aqueous layer was extracted with dichloromethane (30 mL). The combined organic layers were washed with brine (40 mL), dried over Na₂SO₄, filtered, and concentrated to yield crude tert-butyl (3S)-3-(3-oxopropyl)pyrrolidine-1-carboxylate (1.9 g, 98%) as an orange oil. ¹H NMR (400 MHz, CDCl₃) δ 9.80 (s, 1H), 3.62-3.38 (m, 2H), 3.33-3.20 (m, 1H), 2.90-2.87 (m, 1H), 2.50 (t, J=7.5 Hz, 2H), 2.21-2.06 (m, 1H), 2.06-1.95 (m, 1H), 1.80-1.67 (m, 2H), 1.56-1.42 (m, 10H).

### Step 6: tert-Butyl (3S)-3-[(3E)-3-tert-butylsulfinyliminopropyl]pyrrolidine-1-carboxylate

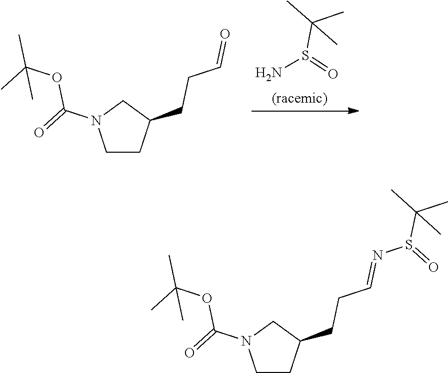

To tert-butyl (3S)-3-(3-oxopropyl)pyrrolidine-1-carboxylate (1.9 g, 7.9410 mmol) dissolved in dichloromethane (20 mL) was added 2-methylpropane-2-sulfinamide (1.5 g, 12.376 mmol), magnesium sulfate (4.8 g, 39.878 mmol) and pyridinium p-toluenesulfonate (145 mg, 0.5747 mmol). The reaction mixture was stirred at room temperature for 16 h. The reaction mixture was filtered then concentrated. The crude was purified by chromatography on silica gel eluted with a gradient of 0 to 70% EtOAc in heptane to give tert-butyl (3S)-3-[(3E)-3-tert-butylsulfinyliminopropyl]pyrrolidine-1-carboxylate (2 g, 68%) as a clear oil. ESI-MS m/z calc. 330.1977, found 231.2 (M Boc+2H=M−99)⁺; Retention time: 1.86 minutes (LC method E).

### Step 7: tert-Butyl (3S)-3-[3-(4-tert-butyl-2-pyridyl)-3-(tert-butylsulfinylamino) propyl]pyrrolidine-1-carboxylate

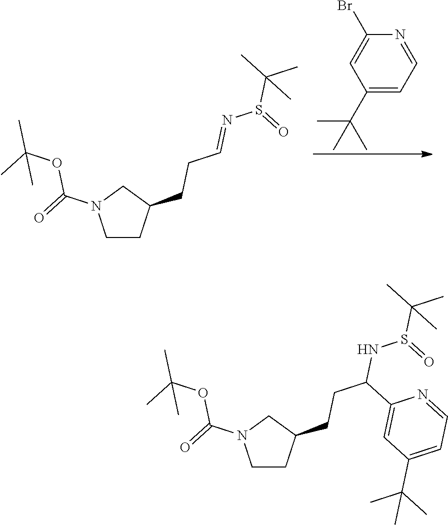

n-Butyllithium in hexanes (2.5 mL of 2.5 M, 6.2500 mmol) was added dropwise to a solution of 2-bromo-4-tert-butyl-pyridine (1.4 g, 6.3428 mmol) in THF (28 mL) at 78° C. The mixture was stirred at 78° C. for 30 min. A solution of tert-butyl (3S)-3-[(3E)-3-tert-butylsulfinyliminopropyl]pyrrolidine-1-carboxylate (2 g, 5.3860 mmol) in THF (6 mL) was added dropwise. The reaction mixture was stirred at 78° C. for 10 min. The reaction mixture was slowly warmed to room temperature and stirred for 4 h. A saturated solution of NH₄Cl (15 mL) was added. The product was extracted with EtOAc (2×20 mL). The combined organic phases were washed with brine (20 mL), dried over sodium sulfite, filtrated and evaporated to dryness. The crude material was purified by silica gel column chromatography using a gradient of 30 to 100% EtOAc in heptanes then 0 to 20% methanol in EtOAc to afford tert-butyl (3S)-3-[3-(4-tert-butyl-2-pyridyl)-3-(tert-butylsulfinylamino)propyl]pyrrolidine-1-carboxylate (1.12 g, 36%) as a yellow oil. ESI-MS m/z calc. 465.3025, found 466.2 (M+1)⁺; Retention time: 1.77 minutes (LC method E).

### Step 8: tert-Butyl (3S)-3-[3-amino-3-(4-tert-butyl-2-pyridyl)propyl]pyrrolidine-1-carboxylate

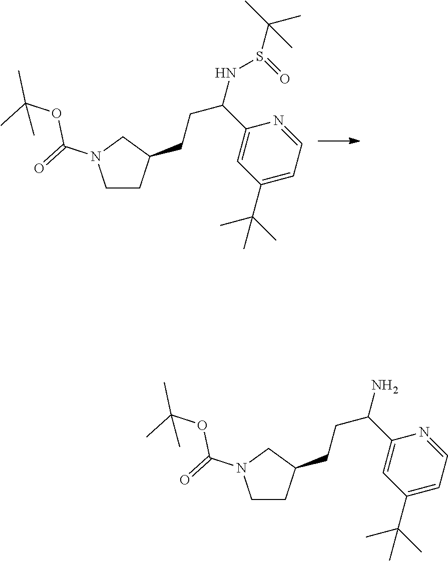 [SMILES: CC(C)(C)OC(=O)N1CC[C@H](CCC(NS(=O)C(C)(C)C)c2cc(C(C)(C)C)ccn2)C1 ; CC(C)(C)OC(=O)N1CC[C@H](CCC(N)c2cc(C(C)(C)C)ccn2)C1]

Iodine (250 mg, 0.9850 mmol) was added to a solution of tert-butyl (3S)-3-[3-(4-tert-butyl-2-pyridyl)-3-(tert-butylsulfinylamino)propyl]pyrrolidine-1-carboxylate (1.12 g, 1.9240 mmol) in THF (8.2 mL) and water (1.6 mL). The reaction was stirred at 60° C. for 2 h. The reaction was diluted with ethyl acetate (30 mL) and washed successively with aqueous 5% sodium bicarbonate solution (2×35 mL) and an aqueous 10% sodium thiosulfate solution (2×35 mL) and brine (50 mL). The organic phase was dried over sodium sulfate, filtered and evaporated to dryness to yield tert-butyl (3S)-3-[3-amino-3-(4-tert-butyl-2-pyridyl)propyl]pyrrolidine-1-carboxylate (900 mg, 98%) as an orange oil. ESI-MS m/z calc. 361.2729, found 362.4 (M+1)⁺; Retention time: 1.45 minutes (LC method E).

### Step 9: tert-Butyl (3S)-3-[3-(4-tert-butyl-2-pyridyl)-3-[(6-sulfamoyl-2-pyridyl)amino]propyl]pyrrolidine-1-carboxylate

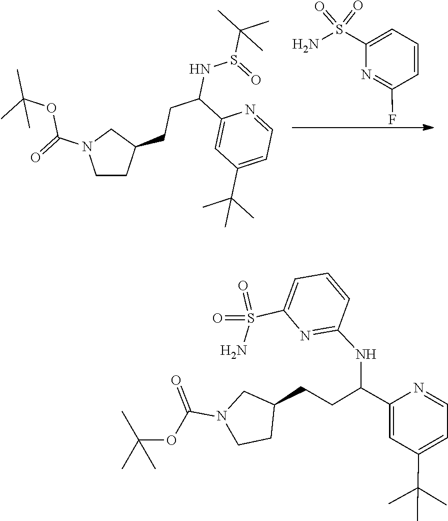 [SMILES: CC(C)(C)OC(=O)N1CC[C@H](CCC(NS(=O)C(C)(C)C)c2cc(C(C)(C)C)ccn2)C1 ; NS(=O)(=O)c1cccc(F)n1 ; CC(C)(C)OC(=O)N1CC[C@H](CCC(Nc2cccc(S(N)(=O)=O)n2)c2cc(C(C)(C)C)ccn2)C1]

To a solution of tert-butyl (3S)-3-[3-amino-3-(4-tert-butyl-2-pyridyl)propyl]pyrrolidine-1-carboxylate (900 mg, 1.8920 mmol) and 6-fluoropyridine-2-sulfonamide (1 g, 5.1915 mmol) in acetonitrile (6 mL) was added DIPEA (890.40 mg, 1.2 mL, 6.8893 mmol). The reaction tube was sealed and the mixture was stirred at 120° C. for 16 h. The reaction was partitioned between ethyl acetate (20 mL) and water (20 mL). The aqueous phase was extracted with ethyl acetate (20 mL). The combined organic layers were washed with brine (50 mL), dried over sodium sulfate, filtered and concentrated. The crude material was purified by reverse phase chromatography on C₁₈ (eluting with 5 to 100% MeCN in water+0.1% HCOOH) to give tert-butyl (3S)-3-[3-(4-tert-butyl-2-pyridyl)-3-[(6-sulfamoyl-2-pyridyl)amino]propyl]pyrrolidine-1-carboxylate (350 mg, 36%) as a light yellow solid. ¹H NMR (400 MHz, CDCl₃) δ 8.46 (d, J=5.4 Hz, 1H), 7.52-7.48 (m, 1H), 7.24-7.15 (m, 2H), 6.58 (d, J=8.6 Hz, 1H), 5.96-5.93 (m, 1H), 5.18-4.85 (m, 2H), 3.67-3.32 (m, 3H), 3.26-3.17 (m, 1H), 2.97-2.84 (m, 1H), 2.82-2.69 (m, 1H), 1.97-1.92 (m, 3H), 1.47-1.39 (m, 13H), 1.31 (s, 9H). ESI-MS m/z calc. 517.2723, found 518.2 (M+1)⁺; Retention time: 1.54 minutes (LC method E).

### Step 10: tert-Butyl (3S)-3-[3-[[6-[(6-tert-butyl-2-fluoro-pyridine-3-carbonyl) sulfamoyl]-2-pyridyl]amino]-3-(4-tert-butyl-2-pyridyl)propyl]pyrrolidine-1-carboxylate

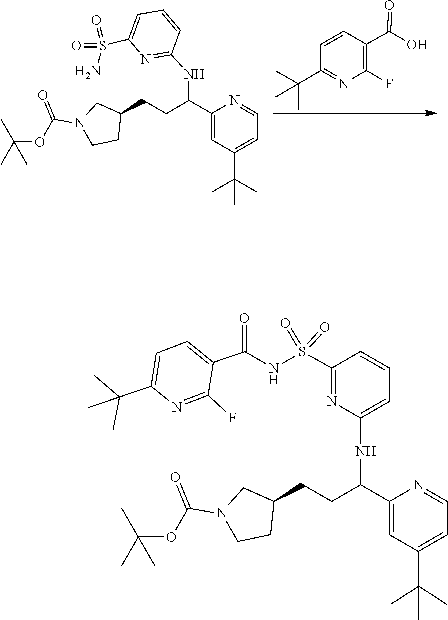

A round-bottomed flask was charged under nitrogen with 6-tert-butyl-2-fluoro-pyridine-3-carboxylic acid (130 mg, 0.6592 mmol) and THF (4 mL). 1,1′-Carbonyldiimidazole (128 mg, 0.7894 mmol) was added and the mixture was stirred under nitrogen at room temperature for 3.5 h. In a separate flask, a solution of tert-butyl (3S)-3-[3-(4-tert-butyl-2-pyridyl)-3-[(6-sulfamoyl-2-pyridyl)amino]propyl]pyrrolidine-1-carboxylate (320 mg, 0.6181 mmol) in THF (3 mL) was prepared under nitrogen and it was added into the activated acid solution. 1,8-Diazabicyclo[5.4.0]undec-7-ene (305.40 mg, 0.3 mL, 2.0061 mmol) was added and the reaction mixture was stirred at room temperature under nitrogen for 3 h. The solvents were removed under reduced pressure and the resulting thick oil was treated with ethyl acetate (30 mL) and water (20 mL). The aqueous phase was extracted with ethyl acetate (20 mL). The combined organic layers were washed with 1 N HCl (20 mL) and dried over sodium sulfate. The crude product was purified by reverse phase chromatography on C₁₈ (eluting with 5 to 100% acetonitrile in water) to give tert-butyl (3S)-3-[3-[[6-[(6-tert-butyl-2-fluoro-pyridine-3-carbonyl)sulfamoyl]-2-pyridyl]amino]-3-(4-tert-butyl-2-pyridyl)propyl]pyrrolidine-1-carboxylate (114 mg, 23%) as a light brown solid. ESI-MS m/z calc. 696.3469, found 697.4 (M+1)⁺; Retention time: 1.9 minutes (LC method E).

### Step 11: 6-tert-Butyl-N-[[6-[[1-(4-tert-butyl-2-pyridyl)-3-[(3S)-pyrrolidin-3-yl]propyl]amino]-2-pyridyl]sulfonyl]-2-fluoro-pyridine-3-carboxamide

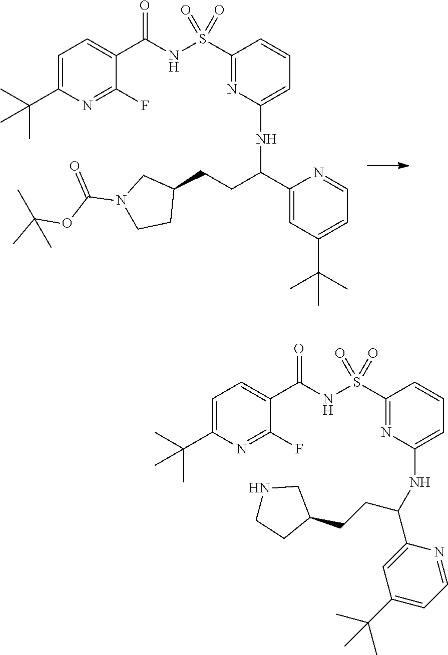

In a round-bottomed flask, tert-butyl (3S)-3-[3-[[6-[(6-tert-butyl-2-fluoro-pyridine-3-carbonyl)sulfamoyl]-2-pyridyl]amino]-3-(4-tert-butyl-2-pyridyl)propyl]pyrrolidine-1-carboxylate (114 mg, 0.1489 mmol) was stirred at room temperature in DCM (2 mL) and 4 M HCl in dioxane (0.7 mL, 2.8000 mmol) for 2 h. Solvent was evaporated to afford 6-tert-butyl-N-[[6-[[1-(4-tert-butyl-2-pyridyl)-3-[(3S)-pyrrolidin-3-yl]propyl]amino]-2-pyridyl]sulfonyl]-2-fluoro-pyridine-3-carboxamide (hydrochloride salt) (110 mg, 105%) as a brown solid. ESI-MS m/z calc. 596.2945, found 597.3 (M+1)⁺; Retention time: 1.4 minutes (LC method E).

### Step 12: (14S)-8-tert-Butyl-17-(4-tert-butylpyridin-2-yl)-2λ⁶-thia-3,9,11,18,23-pentaazatetracyclo[17.3.1.111,14.05,10]tetracosa-1(22),5,7,9,19(23),20-hexaene-2,2,4-trione (Compound 401)

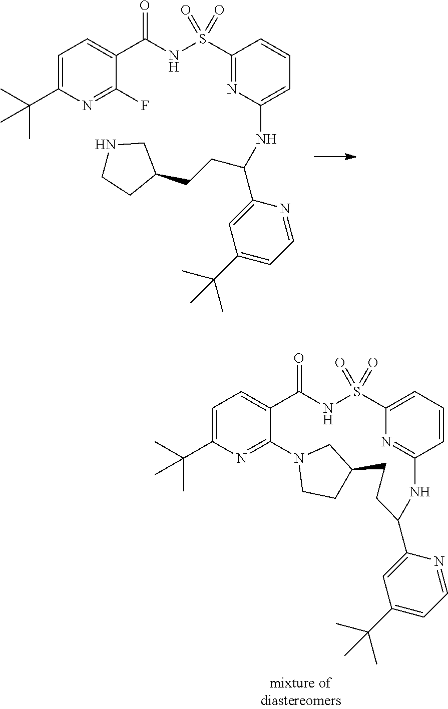 [SMILES: CC(C)(C)c1ccnc(C(CC[C@H]2CCNC2)Nc2cccc(S(=O)(=O)NC(=O)c3ccc(C(C)(C)C)nc3F)n2)c1 ; CC(C)(C)c1ccnc(C2CC[C@H]3CCN(C3)c3nc(C(C)(C)C)ccc3C(=O)NS(=O)(=O)c3cccc(n3)N2)c1]

6-tert-Butyl-N-[[6-[[1-(4-tert-butyl-2-pyridyl)-3-[(3S)-pyrrolidin-3-yl]propyl]amino]-2-pyridyl]sulfonyl]-2-fluoro-pyridine-3-carboxamide (hydrochloride salt) (110 mg, 0.1558 mmol) was dissolved in DMSO (2 mL). Potassium carbonate (280 mg, 2.0260 mmol) was added. The reaction tube was sealed and the mixture was heated at 120° C. for 2 h. The reaction mixture was directly purified by reverse phase chromatography using a C₁₈ column and a gradient of 5 to 100% acetonitrile in water (containing 0.1% HCO₂H), solvent was evaporated to afford a mixture of two diastereomers, (1³3 S)-2⁶6-(tert-butyl)-8-(4-(tert-butyl)pyridin-2-yl)-5-thia-4,7-diaza-2(2,3),6(2,6)-dipyridina-1(1,3)-pyrrolidinacyclodecaphan-3-one 5,5-dioxide (49 mg, 54%); ¹H NMR (400 MHz, DMSO-d₆) δ 8.47-8.35 (m, 1H), 7.66-7.56 (m, 2H), 7.50 (dd, J=8.3, 1.5 Hz, 1H), 7.40-7.33 (m, 1H), 7.26-7.10 (m, 2H), 6.88 (dd, J=8.3, 3.4 Hz, 1H), 6.69 (dd, J=7.9, 4.5 Hz, 1H), 5.41-5.26 (m, 1H), 3.79-3.69 (m, 1H), 3.64-3.58 (m, 1H), 3.54-3.34 (m, 2H), 2.61 (t, J=10.3 Hz, 1H), 2.41-2.30 (m, 1H), 2.27-2.22 (m, 1H), 2.09-1.77 (m, 3H), 1.72-1.49 (m, 2H), 1.35-1.08 (m, 18H). ESI-MS m/z calc. 576.2883, found 577.3 (M+1)⁺; Retention time: 4.07 minutes and Retention time: 4.22 minutes (LC method F).

### Example 141: Preparation of (14S)-8-tert-Butyl-17-(4-tert-butylpyridin-2-yl)-12,12-dimethyl-7-phenyl-2λ⁶-thia-3,9,11,18,23-pentaazatetracyclo[17.3.1.111,14.05,10]tetracosa-1(23),5(10),6,8,19,21-hexaene-2,2,4-trione (Compound 402)

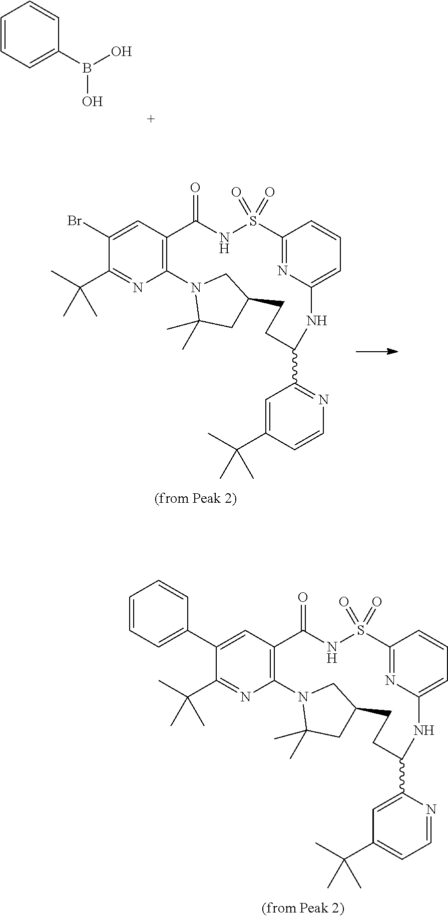 [SMILES: OB(O)c1ccccc1 ; CC(C)(C)c1ccnc(C2CC[C@@H]3CN(c4nc(C(C)(C)C)c(Br)cc4C(=O)NS(=O)(=O)c4cccc(n4)N2)C(C)(C)C3)c1 ; CC(C)(C)c1ccnc(C2CC[C@@H]3CN(c4nc(C(C)(C)C)c(-c5ccccc5)cc4C(=O)NS(=O)(=O)c4cccc(n4)N2)C(C)(C)C3)c1]

In a 1-dram vial, phenylboronic acid (10 mg, 0.08201 mmol), (14S)-7-bromo-8-tert-butyl-17-(4-tert-butylpyridin-2-yl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (20.4 mg, 0.02835 mmol; originating from Peak 2), Pd(PPh₃)₄ (3.5 mg, 0.003029 mmol) and aqueous Na₂CO₃ (100 μL of 2.0 M, 0.2000 mmol) were mixed together with dioxane (200 μL). The resulting mixture was sparged under nitrogen under sonication for 10 min. A pressure-relief cap was fitted onto the vial, and the reaction mixture was stirred vigorously at 110° C. for 21 h. It was cooled to room temperature, then 1 N HCl (0.5 mL) was added, followed by EtOAc (0.5 mL). The phases were vigorously mixed and then allowed to settle into two layers. The organic layer was filtered and purified by reverse-phase preparative chromatography using a C₁₈ column and a gradient eluent of 1 to 99% acetonitrile in water containing 5 mM HCl solution to give a white solid, (14S)-8-tert-butyl-17-(4-tert-butylpyridin-2-yl)-12,12-dimethyl-7-phenyl-2λ⁶-thia-3,9,11,18,23-pentaazatetracyclo[17.3.1.111,14.05,10]tetracosa-1(23),5(10),6,8,19,21-hexaene-2,2,4-trione (7.2 mg, 37%); ¹H NMR (400 MHz, DMSO-d₆) δ 12.70-12.04 (bs, 1H), 8.73-8.56 (m, 1H), 8.06-7.82 (m, 2H), 7.81-7.63 (m, 2H), 7.45-7.35 (m, 3H), 7.34 (s, 1H), 7.33-7.24 (m, 2H), 7.20 (d, J=7.3 Hz, 1H), 6.96 (d, J=8.5 Hz, 1H), 5.50-5.35 (m, 1H), 3.38-3.28 (m, 1H), 2.71 (t, J=10.6 Hz, 1H), 2.43-2.30 (m, 1H), 2.06-1.92 (m, 2H), 1.88 (dd, J=11.8, 5.2 Hz, 1H), 1.78 (dd, J=14.3, 5.3 Hz, 1H), 1.70 (s, 3H), 1.61 (d, J=12.5 Hz, 1H), 1.56 (s, 3H), 1.54-1.43 (m, 1H), 1.31 (s, 9H), 1.15 (s, 9H). [Note: The 1H multiplet at 3.38-3.28 is completely hidden under the water peak; it is only visible with COSY.] ESI-MS m/z calc. 680.3508, found 681.4 (M+1)⁺; Retention time: 1.94 minutes (LC method A).

### Example 142: Preparation of (18S)-21-(4-tert-Butylpyridin-2-yl)-10-(3,3-dimethylbutoxy)-16,16-dimethyl-2λ⁶-thia-3,13,15,22,27-pentaazapentacyclo[21.3.1.115,18.05,14.07,12]octacosa-1(27),5(14),6,8,10,12,23,25-octaene-2,2,4-trione (Compound 403), and (18S)-21-(4-tert-butylpyridin-2-yl)-10-(3,3-dimethylbutoxy)-16,16-dimethyl-2λ⁶-thia-3,13,15,22,27-pentaazapentacyclo[21.3.1.115,18.05,14.07,12]octacosa-1(27),5(14),6,8,10,12,23,25-octaene-2,2,4-trione (Compound 404)

### Step 1: 2-Chloro-7-hydroxyquinoline-3-carbaldehyde

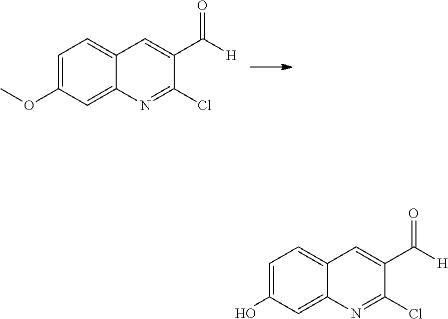

2-Chloro-7-hydroxyquinoline-3-carbaldehyde (5.0 g, 22.56 mmol) in DCM (200 mL) was cooled to −78° C., and boron tribromide (28.2 g, 0.113 mol) was added slowly. The reaction solution warmed to room temperature and stirred for 2 days. The reaction was cooled to 0° C., then aqueous 1 N NaOH was added until the pH reached about 5. Saturated sodium bicarbonate aqueous solution was added until the pH reached 7. Then, the solvent was removed under reduced pressure, and the remaining aqueous solution was extracted with 2-methyl-THF (4×150 mL). The combined organic layer was dried over sodium sulfate, filtered and concentrated. The residue was purified by reverse phase HPLC using 20 to 100% water/acetonitrile (0.1% TFA modifier) to afford 2-chloro-7-hydroxyquinoline-3-carbaldehyde (2.25 g, 48%) as a white solid. ¹H NMR (250 MHz, DMSO-d₆) δ: 11.10 (s, 1H), 10.31 (s, 1H), 8.83 (s, 1H), 8.14 (d, J=8.8 Hz, 1H), 7.29 (dd, J=2.0, 8.8 Hz, 1H), 7.23 (s, 1H). ESI-MS m/z calc. 207.0 found 208.1 (M+1)⁺. Retention time: 2.09 minutes (LC method P).

### Step 2: 7-(3,3-Dimethylbutoxy)-2-chloroquinoline-3-carbaldehyde

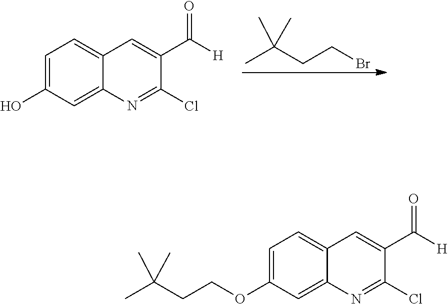

2-Chloro-7-hydroxyquinoline-3-carbaldehyde (2.25 g, 10.88 mmol), 1-bromo-3,3-dimethylbutane (2.34 g, 14.15 mmol) and potassium carbonate (3.46 g, 25.02 mmol) were mixed in DMF (50 mL). The resulting solution was stirred at 70° C. for 3 h, and then cooled to room temperature. Water (150 mL) was added, and solution was extracted with ethyl acetate (3×200 mL). The combined organic layer was washed with brine (50 mL), dried over magnesium sulfate, filtered and concentrated under reduced pressure. The residue was purified by silica gel chromatography using 0 to 100% DCM/hexanes to afford 7-(3,3-dimethylbutoxy)-2-chloroquinoline-3-carbaldehyde (2.40 g, 76%) as a white solid. ¹H NMR (250 MHz, CDCl₃) δ10.51 (s, 1H), 8.66 (s, 1H), 7.83 (d, J=9.0 Hz, 1H), 7.37 (d, J=2.5 Hz, 1H), 7.25 (dd, J=2.5, 9.0 Hz, 1H), 4.20 (t, J=7.3 Hz, 2H), 1.82 (t, J=7.3 Hz, 2H), 1.02 (s, 9H). ESI-MS m/z calc. 291.1 found 292.1 (M+H)⁺; Retention time: 3.94 minutes (LC method P).

### Step 3: 7-(3,3-Dimethylbutoxy)-2-chloroquinoline-3-carboxylic acid

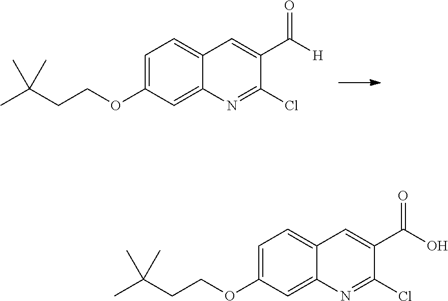 [SMILES: CC(C)(C)CCOc1ccc2cc(C=O)c(Cl)nc2c1 ; CC(C)(C)CCOc1ccc2cc(C(=O)O)c(Cl)nc2c1]

To a suspension of 7-(3,3-dimethylbutoxy)-2-chloroquinoline-3-carbaldehyde (2.40 g, 8.25 mmol) in tert-butyl alcohol (116 mL) was added 2-methyl-2-butene (6.93 g, 99 mmol) followed by an aqueous solution of sodium chlorite (4.98 g, 55.28 mmol) and sodium dihydrogen phosphate monohydrate (6.26 g, 45.38 mmol) in water (39 mL). The resulting solution was stirred at room temperature for 1 h. Then, all the organic solvent was removed under reduced pressure. To the remaining aqueous solution was added 1 N HCl until the pH value was about 4. The solution was filtered and the obtained solid was washed with water to afford 7-(3,3-dimethylbutoxy)-2-chloroquinoline-3-carboxylic acid (2.5 g, 99%) as an off-white solid. ¹H NMR (250 MHz, DMSO-d₆) δ 8.85 (s, 1H), 8.07 (d, J=9.0 Hz, 1H), 7.45 (s, 1H), 7.33 (dd, J=1.8, 9.0 Hz, 1H), 4.23 (t, J=7.3 Hz, 2H), 1.75 (t, J=7.3 Hz, 2H), 1.00 (s, 9H). ESI-MS m/z calc. 307.1 found 308.2 (M+H)⁺. Retention time: 3.35 minutes (LC method P).

### Step 4: tert-Butyl (4S)-4-[3-(4-tert-butyl-2-pyridyl)-3-[[6-[[2-chloro-7-(3,3-dimethylbutoxy)quinoline-3-carbonyl]sulfamoyl]-2-pyridyl]amino]propyl]-2,2-dimethyl-pyrrolidine-1-carboxylate

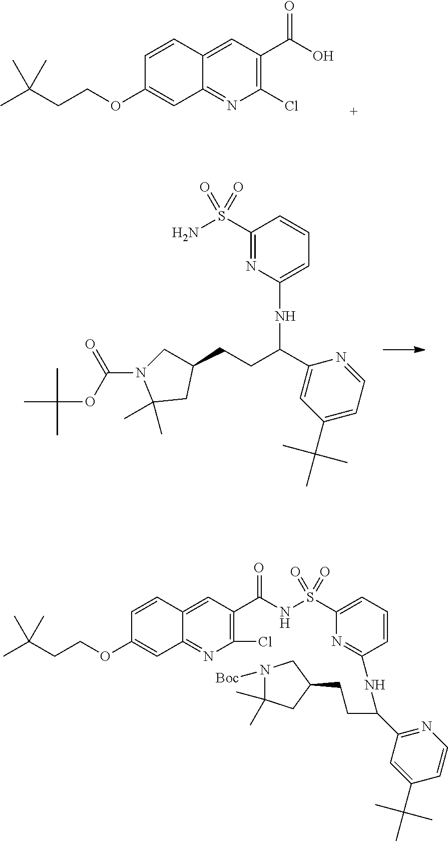 [SMILES: CC(C)(C)CCOc1ccc2cc(C(=O)O)c(Cl)nc2c1 ; CC(C)(C)OC(=O)N1C[C@@H](CCC(Nc2cccc(S(N)(=O)=O)n2)c2cc(C(C)(C)C)ccn2)CC1(C)C ; CC(C)(C)CCOc1ccc2cc(C(=O)NS(=O)(=O)c3cccc(NC(CC[C@@H]4CN(C(=O)OC(C)(C)C)C(C)(C)C4)c4cc(C(C)(C)C)ccn4)n3)c(Cl)nc2c1]

In a 100-mL round-bottomed flask, 2-chloro-7-(3,3-dimethylbutoxy)quinoline-3-carboxylic acid (1.1871 g, 3.857 mmol) was dissolved in THF (20 mL), to which CDI (1.1795 g, 7.274 mmol) was added. The resulting mixture was stirred at room temperature for 17 h. After this time, tert-butyl (4S)-4-[3-(4-tert-butyl-2-pyridyl)-3-[(6-sulfamoyl-2-pyridyl)amino]propyl]-2,2-dimethyl-pyrrolidine-1-carboxylate (2.0292 g, 3.465 mmol) and DBU (2.0 mL, 13.37 mmol) were added, and the resulting mixture was stirred at room temperature for 5.5 h. After this time, the mixture was poured into aqueous 0.1 N HCl (100 mL), and extracted with ethyl acetate (3×100 mL). The combined organic extracts was washed with H₂O (100 mL) and brine (100 mL), then dried over Na₂SO₄, filtered, and evaporated in vacuo. The resulting pink foam was purified by silica gel chromatography (120 g of silica) using a gradient eluent of 0 to 70% EtOAc in hexanes to give a white foam, tert-butyl (4S)-4-[3-(4-tert-butyl-2-pyridyl)-3-[[6-[[2-chloro-7-(3,3-dimethylbutoxy)quinoline-3-carbonyl]sulfamoyl]-2-pyridyl]amino]propyl]-2,2-dimethyl-pyrrolidine-1-carboxylate (1.9468 g, 65%) ESI-MS m/z calc. 834.3905, found 835.4 (M+1)⁺; Retention time: 2.08 minutes (LC method A).

### Step 5: (18S)-21-(4-tert-Butylpyridin-2-yl)-10-(3,3-dimethylbutoxy)-16,16-dimethyl-2λ⁶-thia-3,13,15,22,27-pentaazapentacyclo[21.3.1.115,18.05,14.07,12]octacosa-1(27),5(14),6,8,10,12,23,25-octaene-2,2,4-trione (Compound 403), (185)-21-(4-tert-butylpyridin-2-yl)-10-(3,3-dimethylbutoxy)-16,16-dimethyl-2λ⁶-thia-3,13,15,22,27-pentaazapentacyclo[21.3.1.115,18.05,14.07,12]octacosa-1(27),5(14),6,8,10,12,23,25-octaene-2,2,4-trione (Compound 404)

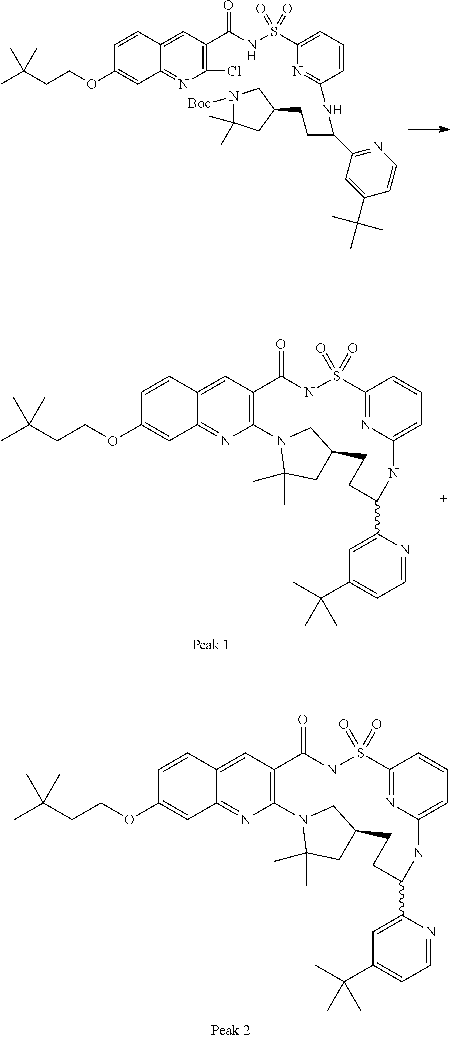 [SMILES: CC(C)(C)CCOc1ccc2cc(C(=O)NS(=O)(=O)c3cccc(NC(CC[C@@H]4CN(C(=O)OC(C)(C)C)C(C)(C)C4)c4cc(C(C)(C)C)ccn4)n3)c(Cl)nc2c1 ; CC(C)(C)CCOc1ccc2cc3c(nc2c1)N1C[C@@H](CCC(c2cc(C(C)(C)C)ccn2)Nc2cccc(n2)S(=O)(=O)NC3=O)CC1(C)C ; CC(C)(C)CCOc1ccc2cc3c(nc2c1)N1C[C@@H](CCC(c2cc(C(C)(C)C)ccn2)Nc2cccc(n2)S(=O)(=O)NC3=O)CC1(C)C]

Stage 1: In a 100-mL round-bottomed flask, tert-butyl (4S)-4-[3-(4-tert-butyl-2-pyridyl)-3-[[6-[[2-chloro-7-(3,3-dimethylbutoxy)quinoline-3-carbonyl]sulfamoyl]-2-pyridyl]amino]propyl]-2,2-dimethyl-pyrrolidine-1-carboxylate (1.9468 g, 2.237 mmol) was dissolved in DCM (20 mL). TFA (5.0 mL, 64.90 mmol) was added, and the resulting solution was allowed to stand at room temperature for 14 h. The mixture was then evaporated in vacuo, dissolved in dioxane (3 mL), and evaporated in vacuo again to give N-[[6-[[1-(4-tert-butyl-2-pyridyl)-3-[(3S)-5,5-dimethylpyrrolidin-3-yl]propyl]amino]-2-pyridyl]sulfonyl]-2-chloro-7-(3,3-dimethylbutoxy)quinoline-3-carboxamide (trifluoroacetate salt) (3 g, >100%).

Stage 2: In a 100-mL round-bottomed flask, the crude product from Stage 1 was dissolved in NMP (20 mL), to which K₂CO₃ (3.1519 g, 22.81 mmol) was added. The resulting mixture was flushed with nitrogen, then stirred at 150° C. for 24 h. The reaction mixture was then cooled to room temperature, quenched with 1 N HCl (50 mL), and extracted with EtOAc (3×50 mL). The combined organic extracts were washed with H₂O (100 mL) and brine (100 mL), then dried over Na₂SO₄, filtered, and evaporated in vacuo. Purification by silica gel chromatography using a gradient eluent of 0 to 70% EtOAc in hexanes gave 3 batches of product (in approximately equal amounts of ˜160 mg) that were all impure. They were re-purified by reverse-phase preparative chromatography using a C₁₈ column and a gradient eluent of 1 to 99% MeCN in H₂O containing 5 mM HCl solution to give 2 separate products:

“Peak 1”: Earlier retention time on reverse-phase UPLC, light yellow solid: (18S)-21-(4-tert-butylpyridin-2-yl)-10-(3,3-dimethylbutoxy)-16,16-dimethyl-2λ⁶-thia-3,13,15,22,27-pentaazapentacyclo[21.3.1.115,18.05,14.07,12]octacosa-1(27),5(14),6,8,10,12,23,25-octaene-2,2,4-trione (187.6 mg, 12%); ¹H NMR (400 MHz, DMSO-d₆) δ 13.00-12.59 (bs, 1H), 8.69 (d, J=6.2 Hz, 1H), 8.18-8.06 (m, 1H), 8.02 (s, 2H), 7.94-7.83 (m, 1H), 7.78 (t, J=8.0 Hz, 1H), 7.70 (d, J=8.9 Hz, 1H), 7.44 (d, J=7.2 Hz, 1H), 7.02 (d, J=8.5 Hz, 1H), 6.98-6.89 (m, 1H), 6.83 (dd, J=8.8, 2.4 Hz, 1H), 5.07-4.85 (m, 1H), 4.12 (td, J=7.4, 2.1 Hz, 2H), 3.52-3.39 (m, 1H), 3.00-2.76 (m, 1H), 2.72-2.58 (m, 1H), 2.38-2.24 (m, 1H), 2.15-2.02 (m, 1H), 1.89 (dd, J=11.6, 5.4 Hz, 1H), 1.85-1.73 (m, 1H), 1.70 (t, J=7.1 Hz, 2H), 1.63 (s, 3H), 1.60 (s, 3H), 1.49 (t, J=11.5 Hz, 1H), 1.31 (s, 9H), 1.22-1.09 (m, 1H), 0.98 (s, 9H). ESI-MS m/z calc. 698.36145, found 699.5 (M+1)⁺; Retention time: 1.64 minutes (LC method A).

“Peak 2”: Later retention time on reverse-phase UPLC, light yellow solid: (18S)-21-(4-tert-butylpyridin-2-yl)-10-(3,3-dimethylbutoxy)-16,16-dimethyl-2λ⁶-thia-3,13,15,22,27-pentaazapentacyclo[21.31115,18.05,14.07,12]octacosa-1(27),5(14),6,8,10,12,23,25-octaene-2,2,4-trione (137.3 mg, 9%); ¹H NMR (400 MHz, DMSO-d₆) δ 12.95-12.37 (bs, 1H), 8.72-8.54 (m, 1H), 8.25 (s, 1H), 8.08-7.82 (m, 2H), 7.82-7.60 (m, 3H), 7.22 (d, J=7.1 Hz, 1H), 7.01-6.89 (m, 2H), 6.84 (dd, J=8.8, 2.4 Hz, 1H), 5.60-5.37 (m, 1H), 4.19-4.08 (m, 2H), 3.60-3.34 (m, 1H), 2.75 (t, J=10.2 Hz, 1H), 2.42-2.27 (m, 1H), 2.09-1.94 (m, 2H), 1.88 (dd, J=11.8, 5.2 Hz, 1H), 1.84-1.75 (m, 1H), 1.75 (s, 3H), 1.72 (t, J=7.1 Hz, 2H), 1.66-1.55 (m, 1H), 1.60 (s, 3H), 1.55-1.42 (m, 1H), 1.32 (s, 9H), 0.99 (s, 9H). ESI-MS m/z calc. 698.36145, found 699.6 (M+1)⁺; Retention time: 1.75 minutes (LC method A).

### Example 143: Preparation of (14S)-8-tert-Butyl-12,12-dimethyl-17-[(1E)-2-phenylethenyl]-2λ⁶-thia-3,9,11,18,23-pentaazatetracyclo[17.3.1.111,14.05,10]tetracosa-1(23),5(10),6,8,19,21-hexaene-2,2,4-trione (Compound 405)

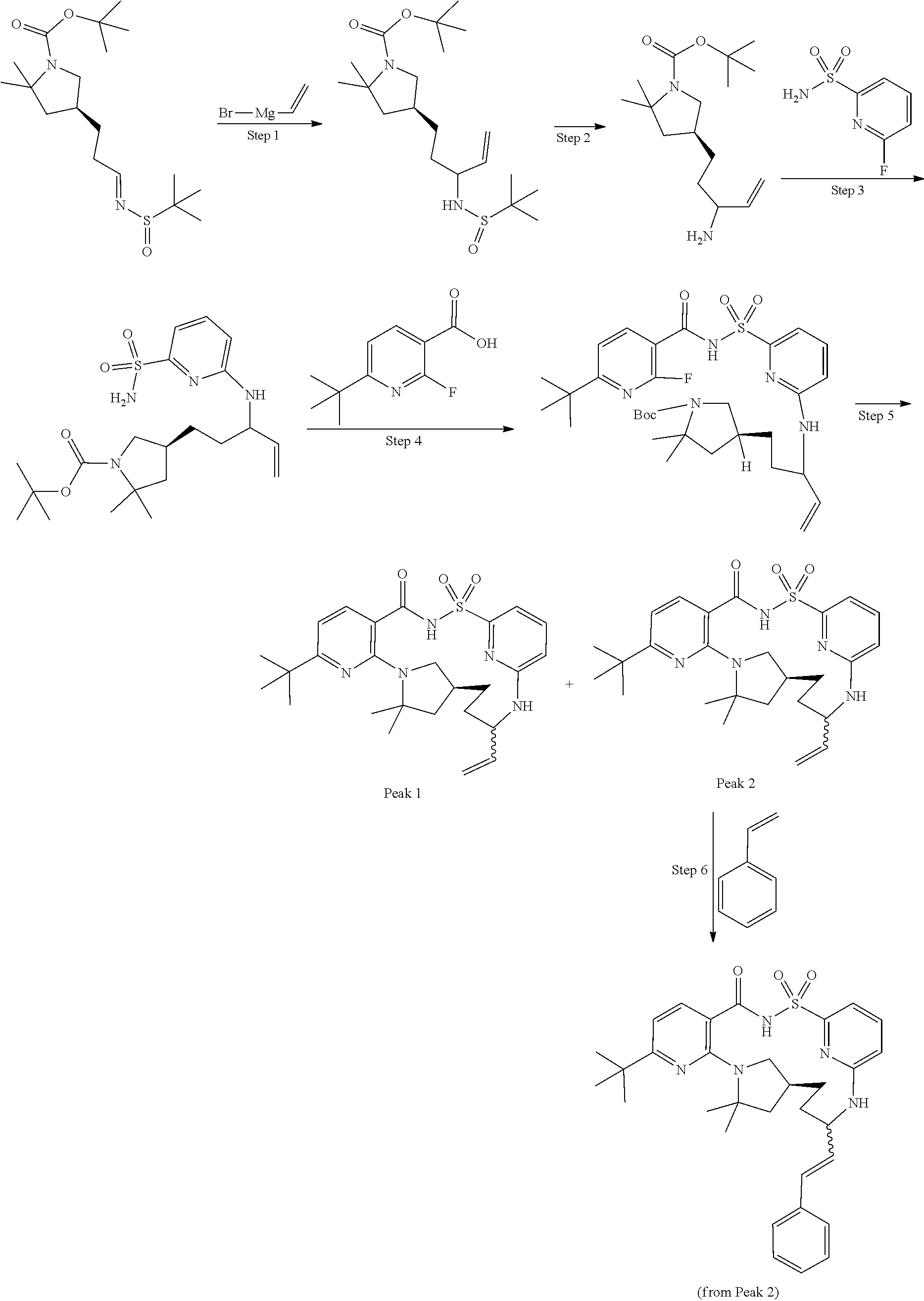

### Step 1: tert-Butyl (4S)-4-[3-(tert-butylsulfinylamino)pent-4-enyl]-2,2-dimethyl-pyrrolidine-1-carboxylate

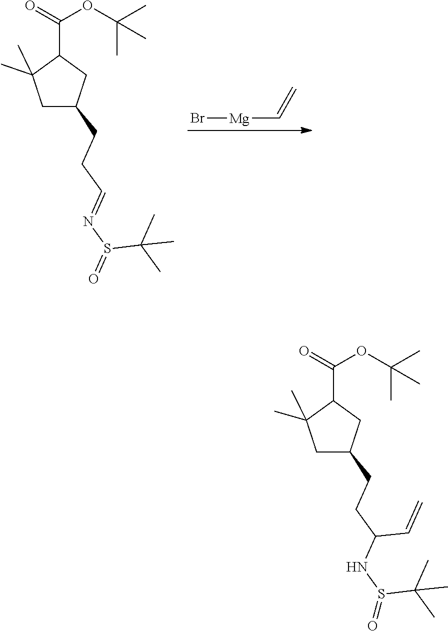

To a solution of tert-butyl (4S)-4-[(3E)-3-tert-butylsulfinyliminopropyl]-2,2-dimethyl-pyrrolidine-1-carboxylate (6 g, 16.735 mmol) in THF (60.000 mL) at 78° C. was dropwise added a solution of vinylmagnesium bromide in THF (34 mL of 1 M, 34.000 mmol). The reaction was stirred at 78° C. for 30 min and then slowly warmed to room temperature over 3 h. Aqueous sat. NH₄Cl was added (50 mL) and the product was extracted with EtOAc (3×50 mL). The combined organic layers were washed with brine (50 mL), dried over sodium sulfate, filtered and concentrated under reduced pressure. The crude product was purified by silica gel chromatography (DCM/MeOH, 0 to 20%) to give tert-butyl(4S)-4-[(3-(tert-butylsulfinylamino)pent-4-enyl]-2,2-dimethyl-pyrrolidine-1-carboxylate (6.05 g, 91%) as a yellow oil. ESI-MS m/z calc. 386.2603, found 387.2 (M+1)⁺; Retention time: 1.976 minutes (LC method E).

### Step 2: tert-butyl (4S)-4-(3-aminopent-4-enyl)-2,2-dimethyl-pyrrolidine-1-carboxylate

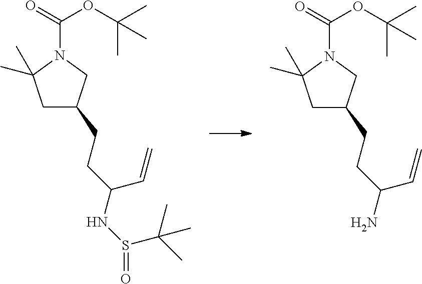 [SMILES: C=CC(CC[C@@H]1CN(C(=O)OC(C)(C)C)C(C)(C)C1)NS(=O)C(C)(C)C ; C=CC(N)CC[C@@H]1CN(C(=O)OC(C)(C)C)C(C)(C)C1]

To a solution of tert-butyl (4S)-4-[3-(tert-butylsulfinylamino)pent-4-enyl]-2,2-dimethyl-pyrrolidine-1-carboxylate (6.5 g, 16.814 mmol) in THF (55 mL) and water (8.6 mL) was added iodine (1.3 g, 0.2637 mL, 5.1220 mmol) and the mixture was stirred at 50° C. for 18 h. The reaction mixture was cooled to room temperature and extracted with DCM (3×25 mL). The combined organic layers were washed with sat. aq. NaHCO₃ solution (50 mL) and 10% aq. sodium thiosulfate (50 mL). The combined organic layers were washed with brine (50 mL), dried over sodium sulfate, filtered and concentrated under reduced pressure to afford tert-butyl (4S)-4-(3-aminopent-4-enyl)-2,2-dimethyl-pyrrolidine-1-carboxylate. ESI-MS m/z calc. 282.23074, found 283.4 (M+1)⁺; Retention time: 1.37 minutes (LC method E).

### Step 3: tert-Butyl (4S)-2,2-dimethyl-4-[3-[(6-sulfamoyl-2-pyridyl)amino]pent-4-enyl]pyrrolidine-1-carboxylate

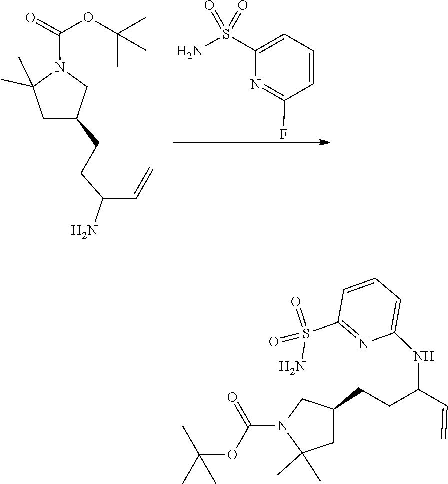 [SMILES: C=CC(N)CC[C@@H]1CN(C(=O)OC(C)(C)C)C(C)(C)C1 ; NS(=O)(=O)c1cccc(F)n1 ; C=CC(CC[C@@H]1CN(C(=O)OC(C)(C)C)C(C)(C)C1)Nc1cccc(S(N)(=O)=O)n1]

To a solution of tert-butyl (4S)-4-(3-aminopent-4-enyl)-2,2-dimethyl-pyrrolidine-1-carboxylate (4.75 g, 16.819 mmol) and 6-fluoropyridine-2-sulfonamide (4.5 g, 25.544 mmol) in acetonitrile (30 mL) was added DIPEA (7.6426 g, 10.3 mL, 59.134 mmol). The mixture was stirred at 120° C. for 48 h. The reaction mixture was then cooled to room temperature and was partitioned between ethyl acetate (10 mL) and water (10 mL). The aqueous phase was extracted with ethyl acetate (2×40 mL). The combined organic layers were washed with brine (40 mL), dried over sodium sulfate, filtered and concentrated to provide crude product. The crude material was purified by reverse phase chromatography eluting with a gradient of 5% to 80% MeOH/H₂O (+0.1% formic acid as a modifier) to afford tert-butyl (4S)-2,2-dimethyl-4-[3-[(6-sulfamoyl-2-pyridyl)amino]pent-4-enyl]pyrrolidine-1-carboxylate (2.54 g, 33%) as a beige solid. ESI-MS m/z calc. 438.2301, found 383.2 (M-tBu+2H=M−55)⁺; Retention time: 4.28 minutes (LC method F).

### Step 4: tert-Butyl (4S)-4-[3-[[6-[(6-tert-butyl-2-fluoro-pyridine-3-carbonyl)sulfamoyl]-2-pyridyl]amino]pent-4-enyl]-2,2-dimethyl-pyrrolidine-1-carboxylate

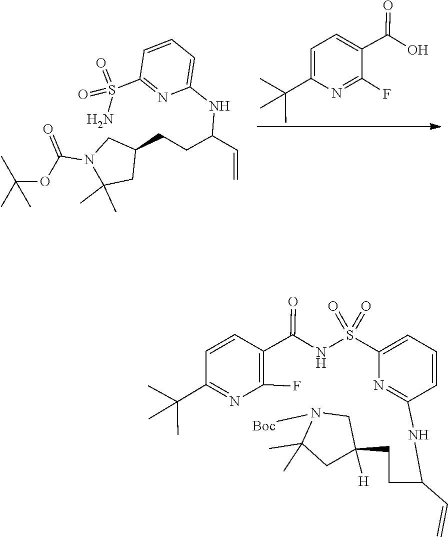 [SMILES: C=CC(CC[C@@H]1CN(C(=O)OC(C)(C)C)C(C)(C)C1)Nc1cccc(S(N)(=O)=O)n1 ; CC(C)(C)c1ccc(C(=O)O)c(F)n1 ; C=CC(CC[C@@H]1CN(C(=O)OC(C)(C)C)C(C)(C)C1)Nc1cccc(S(=O)(=O)NC(=O)c2ccc(C(C)(C)C)nc2F)n1]

In a 100-mL round-bottomed flask, 6-tert-butyl-2-fluoro-pyridine-3-carboxylic acid (2.8562 g, 14.48 mmol) was dissolved in THF (50 mL), to which CDI (3.5552 g, 21.93 mmol) was added. The resulting mixture was stirred at room temperature for 14 h. After this time, tert-butyl (4S)-2,2-dimethyl-4-[3-[(6-sulfamoyl-2-pyridyl)amino]pent-4-enyl]pyrrolidine-1-carboxylate (4.4 g, 9.681 mmol) and DBU (6 mL, 40.12 mmol) were added, and the resulting mixture was stirred at room temperature for 6 h. After this time, the mixture was poured into aqueous 0.1 N HCl (80 mL) and extracted with ethyl acetate (3×80 mL). The combined organic extracts were washed with H₂O (100 mL) and brine (100 mL), then dried over Na₂SO₄, filtered, and evaporated in vacuo. Purification by silica gel chromatography (220 g of silica) using a gradient eluent of 0 to 70% EtOAc in hexanes gave a white foam, tert-butyl (4S)-4-[3-[[6-[(6-tert-butyl-2-fluoro-pyridine-3-carbonyl)sulfamoyl]-2-pyridyl]amino]pent-4-enyl]-2,2-dimethyl-pyrrolidine-1-carboxylate (3.9345 g, 66%); ESI-MS m/z calc. 617.30475, found 618.5 (M+1)⁺; Retention time: 2.19 minutes and 2.21 minutes (LC method A).

### Step 5: (14S)-8-tert-butyl-17-ethenyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione, (14S)-8-tert-butyl-17-ethenyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione

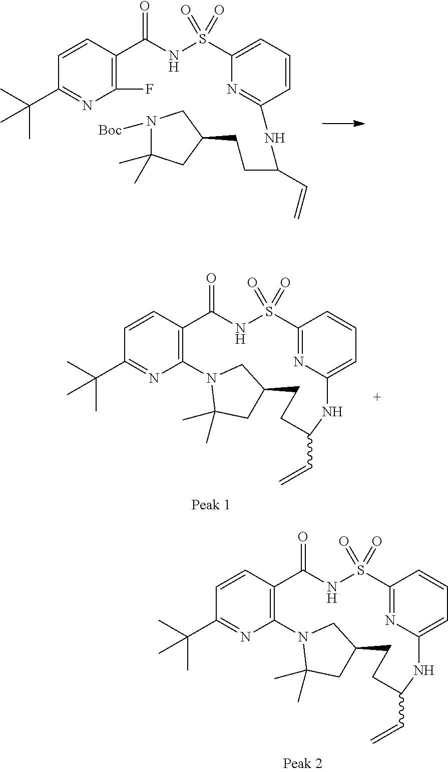 [SMILES: C=CC(CC[C@@H]1CN(C(=O)OC(C)(C)C)C(C)(C)C1)Nc1cccc(S(=O)(=O)NC(=O)c2ccc(C(C)(C)C)nc2F)n1 ; C=CC1CC[C@@H]2CN(c3nc(C(C)(C)C)ccc3C(=O)NS(=O)(=O)c3cccc(n3)N1)C(C)(C)C2 ; C=CC1CC[C@@H]2CN(c3nc(C(C)(C)C)ccc3C(=O)NS(=O)(=O)c3cccc(n3)N1)C(C)(C)C2]

Stage 1: In a 100-mL round-bottomed flask, tert-butyl (4S)-4-[3-[[6-[(6-tert-butyl-2-fluoro-pyridine-3-carbonyl)sulfamoyl]-2-pyridyl]amino]pent-4-enyl]-2,2-dimethyl-pyrrolidine-1-carboxylate (3.9345 g, 6.369 mmol) was dissolved in DCM (30 mL). TFA (5 mL, 64.90 mmol) was added, and the resulting solution was stirred at room temperature for 12 h. The mixture was then evaporated in vacuo, diluted with dioxane, and evaporated in vacuo again. This gave a brown oil, 6 g (>100% yield).

Stage 2: In a 100-mL round bottomed flask, the crude product from Stage 1 was dissolved in NMP (40 mL), to which K₂CO₃ (9.123 g, 66.01 mmol) was added. The resulting mixture was flushed with nitrogen, then stirred at 150° C. for 27 h. After cooling to room temperature, the resulting mixture was poured into 1 N HCl (50 mL), then extracted with EtOAc (3×50 mL). The combined organic extracts were washed with H₂O (2×50 mL) and brine (50 mL), then dried over Na₂SO₄, filtered, and evaporated in vacuo. Several purifications by silica gel chromatography gave two separate products.

“Peak 1”, which elutes earlier from a silica column: (14S)-8-tert-butyl-17-ethenyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (691.0 mg, 22%); ¹H NMR (400 MHz, DMSO-d₆) δ 12.30 (s, 1H), 7.59 (dd, J=8.5, 7.3 Hz, 1H), 7.45 (d, J=7.9 Hz, 1H), 7.37 (d, J=7.6 Hz, 1H), 7.19 (d, J=7.3 Hz, 1H), 6.75 (d, J=8.5 Hz, 1H), 6.62 (d, J=7.9 Hz, 1H), 6.04 (dt, J=17.2, 9.1 Hz, 1H), 5.19 (d, J=17.3 Hz, 1H), 5.05 (d, J=10.3 Hz, 1H), 4.28-4.01 (m, 1H), 3.21-3.07 (m, 1H), 3.06-2.88 (m, 1H), 2.31-2.19 (m, 1H), 2.19-1.98 (m, 1H), 1.84 (dd, J=11.8, 5.7 Hz, 1H), 1.76-1.64 (m, 1H), 1.58 (s, 3H), 1.58-1.52 (m, 1H), 1.52 (s, 3H), 1.52-1.43 (m, 1H), 1.27 (s, 9H), 1.16-1.06 (m, 1H). ESI-MS m/z calc. 497.24606, found 498.2 (M+1)⁺; Retention time: 2.06 minutes (LC method A).

“Peak 2”, which elutes later from a silica column: (14S)-8-tert-butyl-17-ethenyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (798.1 mg, 25%); ¹H NMR (400 MHz, DMSO-d₆) δ 12.49 (s, 1H), 7.66-7.52 (m, 2H), 7.14 (d, J=8.9 Hz, 1H), 7.07 (d, J=7.2 Hz, 1H), 6.78 (d, J=8.4 Hz, 1H), 6.63 (d, J=8.0 Hz, 1H), 5.84 (ddd, J=17.1, 10.2, 4.3 Hz, 1H), 5.14 (dt, J=17.2, 1.8 Hz, 1H), 4.94 (dt, J=10.2, 1.8 Hz, 1H), 4.70-4.54 (m, 1H), 3.07 (dd, J=10.3, 6.8 Hz, 1H), 2.68 (t, J=10.3 Hz, 1H), 2.22-2.07 (m, 1H), 1.81 (dd, J=11.7, 5.2 Hz, 1H), 1.69-1.65 (m, 2H), 1.62 (s, 3H), 1.62-1.48 (m, 2H), 1.48 (s, 3H), 1.41-1.27 (m, 1H), 1.27 (s, 9H). ESI-MS m/z calc. 497.24606, found 498.2 (M+1)⁺; Retention time: 2.07 minutes (LC method A).

### Step 6: (14S)-8-tert-Butyl-12,12-dimethyl-17-[(1E)-2-phenylethenyl]-2λ⁶-thia-3,9,11,18,23-pentaazatetracyclo[17.3.1.111,14.05,10]tetracosa-1(23),5(10),6,8,19,21-hexaene-2,2,4-trione (Compound 405)

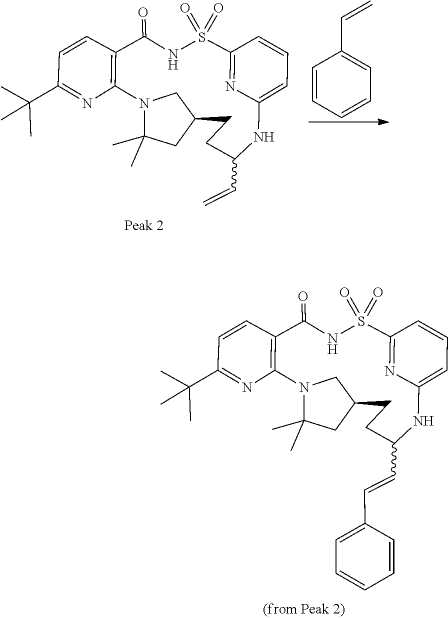

In a 1-dram vial, Peak 2 of (14S)-8-tert-butyl-17-ethenyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (30 mg, 0.06028 mmol) was dissolved in DCE (500 μL), to which styrene (20 μL, 0.1746 mmol) and Hoveyda-Grubbs 2^{nd} generation catalyst (2.0 mg, 0.003192 mmol) were added. The resulting mixture was stirred under nitrogen atmosphere at room temperature for 17.5 h, then at 55° C. for 44 h. It was then cooled to room temperature, then a second portion of Hoveyda-Grubbs 2^{nd} generation catalyst (2.0 mg, 0.003192 mmol) was added. The resulting mixture was stirred under nitrogen atmosphere at 55° C. for 28 h. It was cooled to room temperature and filtered through a short pipet of Celite and silica (rinsed with DCM). Evaporation in vacuo, then purification by reverse-phase preparative chromatography using a C₁₈ column and a gradient eluent of 1 to 99% MeCN in H₂O containing 5 mM HCl solution gave (14S)-8-tert-butyl-12,12-dimethyl-17-[(1E)-2-phenylethenyl]-2λ⁶-thia-3,9,11,18,23-pentaazatetracyclo[17.3.1.111,14.05,10]tetracosa-1(23),5(10),6,8,19,21-hexaene-2,2,4-trione (2.6 mg, 8%); ¹H NMR (400 MHz, DMSO-d₆) δ 12.51 (s, 1H), 7.63 (dd, J=8.5, 7.2 Hz, 1H), 7.61 (d, J=8.0 Hz, 1H), 7.40-7.33 (m, 2H), 7.32-7.23 (m, 3H), 7.22-7.16 (m, 1H), 7.09 (d, J=7.2 Hz, 1H), 6.83 (d, J=8.4 Hz, 1H), 6.65 (d, J=8.0 Hz, 1H), 6.50 (d, J=15.9 Hz, 1H), 6.30 (dd, J=15.9, 4.8 Hz, 1H), 4.86-4.74 (m, 1H), 3.12 (dd, J=10.3, 6.7 Hz, 1H), 2.71 (t, J=10.5 Hz, 1H), 2.25-2.10 (m, 1H), 1.83 (dd, J=11.8, 5.2 Hz, 1H), 1.78-1.65 (m, 3H), 1.64 (s, 3H), 1.55 (t, J=12.5 Hz, 1H), 1.50 (s, 3H), 1.44-1.35 (m, 1H), 1.28 (s, 9H); ESI-MS m/z calc. 573.27734, found 574.3 (M+1)⁺; Retention time: 2.26 minutes (LC method A).

### Example 144: Preparation of (14S)-8-tert-Butyl-12,12-dimethyl-17-[(1E)-2-(pyridin-3-yl)ethenyl]-2λ⁶-thia-3,9,11,18,23-pentaazatetracyclo[17.3.1.111,14.05,10]tetracosa-1(23),5(10),6,8,19,21-hexaene-2,2,4-trione (Compound 406)

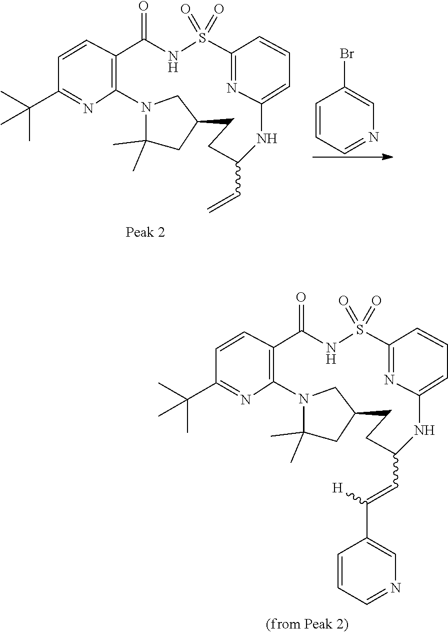

In a 1-dram vial, Peak 2 of (14S)-8-tert-butyl-17-ethenyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (60 mg, 0.1206 mmol) was dissolved in MeCN (1.5 mL), to which 3-bromopyridine (39.1 mg, 0.2475 mmol), Pd(OAc)₂ (3.0 mg, 0.01336 mmol), P(o-tolyl)₃ (3.0 mg, 0.009857 mmol) and Et₃N (50 μL, 0.3587 mmol) were added. The resulting mixture was sparged with a stream of nitrogen gas for 5 min, and then stirred at 80° C. under nitrogen for 22.5 h. It was then cooled to room temperature, diluted with MeOH (1 mL), filtered, and purified by reverse-phase preparative chromatography using a C₁₈ column and a gradient eluent of 1 to 70% MeCN in H₂O containing 5 mM HCl solution to give (14S)-8-tert-butyl-12,12-dimethyl-17-[(1E)-2-(pyridin-3-yl)ethenyl]-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (31.8 mg, 43%); ¹H NMR (400 MHz, DMSO-d₆) δ 12.52 (s, 1H), 8.91 (d, J=2.1 Hz, 1H), 8.72-8.62 (m, 1H), 8.57-8.41 (m, 1H), 7.89 (dd, J=8.2, 5.4 Hz, 1H), 7.66 (dd, J=8.5, 7.3 Hz, 1H), 7.61 (d, J=8.0 Hz, 1H), 7.39 (d, J=8.4 Hz, 1H), 7.13 (d, J=7.2 Hz, 1H), 6.87 (d, J=8.5 Hz, 1H), 6.79 (dd, J=16.0, 4.2 Hz, 1H), 6.70-6.59 (m, 2H), 4.90-4.79 (m, 1H), 3.10 (dd, J=10.3, 6.7 Hz, 1H), 2.70 (t, J=10.3 Hz, 1H), 2.25-2.11 (m, 1H), 1.84 (dd, J=11.8, 5.3 Hz, 1H), 1.79-1.67 (m, 3H), 1.65 (s, 3H), 1.56 (t, J=12.3 Hz, 1H), 1.50 (s, 3H), 1.45-1.34 (m, 1H), 1.28 (s, 9H). ESI-MS m/z calc. 574.2726, found 575.2 (M+1)⁺; Retention time: 1.62 minutes (LC method A).

### Example 145: Preparation of (14S)-8-tert-Butyl-12,12-dimethyl-17-[(pyridin-2-yl)methyl]-2λ⁶-thia-3,9,11,18,23-pentaazatetracyclo[17.3.1.111,14.05,10]tetracosa-1(23),5(10),6,8,19,21-hexaene-2,2,4-trione (Compound 407) and (14S)-8-tert-butyl-12,12-dimethyl-17-[(pyridin-2-yl)methyl]-2λ⁶-thia-3,9,11,18,23-pentaazatetracyclo[17.3.1.111,14.05,10]tetracosa-1(23),5(10),6,8,19,21-hexaene-2,2,4-trione (Compound 408)

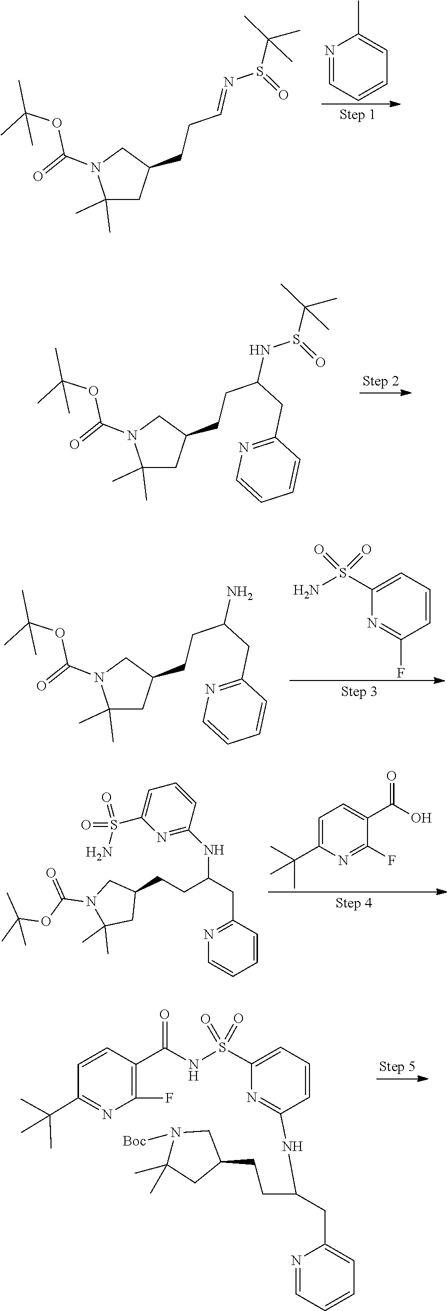

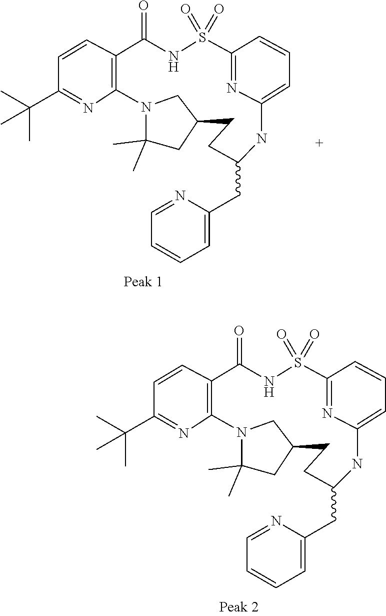 [SMILES: CC(C)(C)c1ccc2c(n1)N1C[C@@H](CCC(Cc3ccccn3)Nc3cccc(n3)S(=O)(=O)NC2=O)CC1(C)C ; CC(C)(C)c1ccc2c(n1)N1C[C@@H](CCC(Cc3ccccn3)Nc3cccc(n3)S(=O)(=O)NC2=O)CC1(C)C]

### Step 1: tert-Butyl (4S)-4-[3-(tert-butylsulfinylamino)-4-(2-pyridyl)butyl]-2,2-dimethyl-pyrrolidine-1-carboxylate

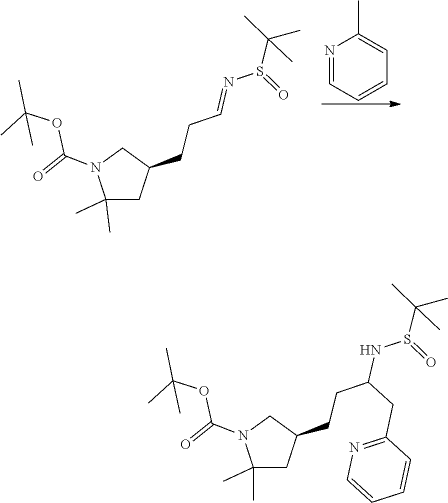

To a solution of 2-methylpyridine (3.9 g, 41.041 mmol) in THF (60 mL) at 0° C. was added a solution of n-butyllithium in hexanes (17 mL of 2.5 M, 42.500 mmol) dropwise. The resulting solution was allowed to warm to room temperature gradually and stirred for 3 h. Then, the mixture was cooled to 78° C., to which tert-butyl (4S)-4-[(3E)-3-tert-butylsulfinyliminopropyl]-2,2-dimethyl-pyrrolidine-1-carboxylate (10 g, 27.333 mmol) in THF (60 mL) was added dropwise. The resulting mixture was stirred at 78° C. for 10 min and then allowed to warm to 0° C. over 30 min. Then treated with saturated aqueous NH₄Cl solution (100 mL). The product was extracted with DCM (3×150 mL). The combined extracts were washed with water (150 mL), dried with anhydrous sodium sulfate, filtered and concentrated under reduced pressure. The crude material was directly purified by reverse phase chromatography (C₁₈) using a gradient of 5 to 100% acetonitrile in water. Acetonitrile was evaporated and the aqueous solution was extracted with EtOAc (3×150 mL). The combined extracts were washed with brine (150 mL), dried with anhydrous sodium sulfate, filtered and concentrated under reduced pressure to give tert-butyl (4S)-4-[3-(tert-butylsulfinylamino)-4-(2-pyridyl)butyl]-2,2-dimethyl-pyrrolidine-1-carboxylate (11.5 g, 89%) as a yellow oil. ESI-MS m/z calc. 451.2869, found 452.4 (M+1)⁺; Retention time: 1.55 minutes and 1.58 minutes (LC method E).

### Step 2: tert-Butyl (4S)-4-[3-amino-4-(2-pyridyl)butyl]-2,2-dimethyl-pyrrolidine-1-carboxylate

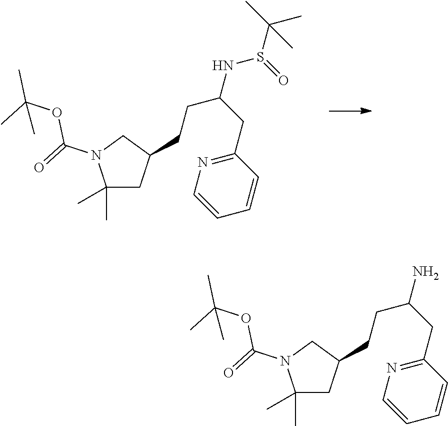

Iodine (3 g, 11.820 mmol) was added to a solution of tert-butyl (4S)-4-[3-(tert-butylsulfinylamino)-4-(2-pyridyl)butyl]-2,2-dimethyl-pyrrolidine-1-carboxylate (11.5 g, 24.392 mmol) in THF (80 mL) and water (17 mL). The reaction was stirred at 60° C. for 2 h. The reaction was diluted with ethyl acetate (125 mL) and washed successively with aqueous 5% sodium bicarbonate solution (2×125 mL), aqueous 10% sodium thiosulfate solution (2×125 mL) and brine (150 mL). The organic phase was dried over sodium sulfate, filtered and evaporated to dryness to yield crude tert-butyl (4S)-4-[3-amino-4-(2-pyridyl)butyl]-2,2-dimethyl-pyrrolidine-1-carboxylate (8.33 g, 80%) as an orange oil. ESI-MS m/z calc. 347.25726, found 348.4 (M+1)⁺; Retention time: 1.37 minutes (LC method E).

### Step 3: tert-Butyl (4S)-2,2-dimethyl-4-[4-(2-pyridyl)-3-[(6-sulfamoyl-2-pyridyl)amino]butyl]pyrrolidine-1-carboxylate

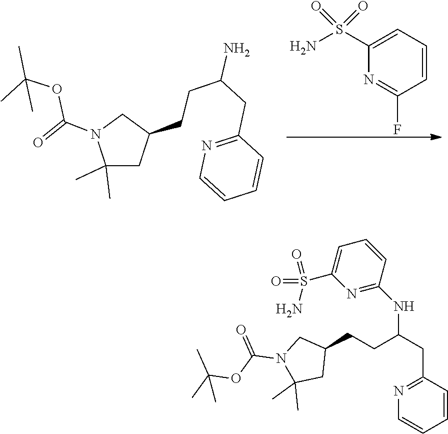 [SMILES: CC(C)(C)OC(=O)N1C[C@@H](CCC(N)Cc2ccccn2)CC1(C)C ; NS(=O)(=O)c1cccc(F)n1 ; CC(C)(C)OC(=O)N1C[C@@H](CCC(Cc2ccccn2)Nc2cccc(S(N)(=O)=O)n2)CC1(C)C]

To a solution of tert-butyl (4S)-4-[3-amino-4-(2-pyridyl)butyl]-2,2-dimethyl-pyrrolidine-1-carboxylate (8.3 g, 19.586 mmol) and 6-fluoropyridine-2-sulfonamide (11 g, 62.440 mmol) in acetonitrile (65 mL) was added DIPEA (11.130 g, 15 mL, 86.117 mmol). The reaction tube was sealed and the mixture was stirred at 120° C. for 16 h. The reaction was partitioned between ethyl acetate (120 mL) and water (120 mL). The aqueous phase was extracted with ethyl acetate (2×100 mL). The combined organic layers were washed with brine (150 mL), dried over sodium sulfate, filtered and concentrated. The crude product was purified by reverse phase chromatography on C₁₈ (eluting with 5 to 100% MeCN in water). The product fractions were combined. MeCN was evaporated, and the aqueous solution was extracted with ethyl acetate (2×100 ml). The combined organic layers were washed with brine (100 mL), dried over sodium sulfate, filtered and concentrated to give tert-butyl (4S)-2,2-dimethyl-4-[4-(2-pyridyl)-3-[(6-sulfamoyl-2-pyridyl)amino]butyl]pyrrolidine-1-carboxylate (5.2 g, 50%) as a light brown solid. ¹H NMR (400 MHz, DMSO-d₆) δ 8.50-8.43 (m, 1H), 7.66 (t, J=7.6 Hz, 1H), 7.54-7.47 (m, 1H), 7.33 (d, J=7.6 Hz, 1H), 7.21-7.16 (m, 1H), 7.11 (br. s., 2H), 6.94 (d, J=6.8 Hz, 2H), 6.61 (d, J=8.3 Hz, 1H), 4.28 (br. s., 1H), 3.52-3.40 (m, 1H), 3.05-3.00 (m, 1H), 2.88-2.83 (m, 1H), 2.76-2.65 (m, 1H), 2.01-1.98 (m, 1H), 1.86-1.71 (m, 1H), 1.58-1.43 (m, 2H), 1.41-1.24 (m, 15H), 1.23-1.14 (m, 3H). ESI-MS m/z calc. 503.25662, found 504.3 (M+1)⁺; Retention time: 2.77 minutes (LC method F).

### Step 4: tert-Butyl (4S)-4-[3-[[6-[(6-tert-butyl-2-fluoro-pyridine-3-carbonyl)sulfamoyl]-2-pyridyl]amino]-4-(2-pyridyl)butyl]-2,2-dimethyl-pyrrolidine-1-carboxylate

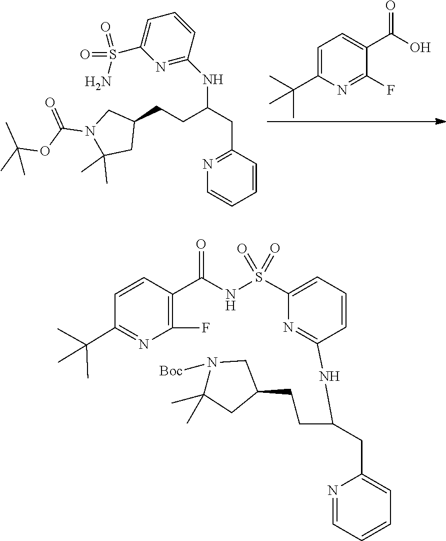 [SMILES: CC(C)(C)OC(=O)N1C[C@@H](CCC(Cc2ccccn2)Nc2cccc(S(N)(=O)=O)n2)CC1(C)C ; CC(C)(C)c1ccc(C(=O)O)c(F)n1 ; CC(C)(C)OC(=O)N1C[C@@H](CCC(Cc2ccccn2)Nc2cccc(S(=O)(=O)NC(=O)c3ccc(C(C)(C)C)nc3F)n2)CC1(C)C]

In a 20-mL vial, 6-tert-butyl-2-fluoro-pyridine-3-carboxylic acid (220 mg, 1.116 mmol) was mixed with DCM (2.0 mL) and cooled to 0° C., to which DMF (10 μL, 0.1291 mmol) and oxalyl chloride (200 μL, 2.293 mmol) were added. The resulting solution was warmed to room temperature over 4 h. Upon verifying that the acyl chloride has indeed formed (with a small sample quench using morpholine), the mixture was evaporated in vacuo.

Separately, in a 100-mL round-bottomed flask, a solution of tert-butyl (4S)-2,2-dimethyl-4-[4-(2-pyridyl)-3-[(6-sulfamoyl-2-pyridyl)amino]butyl]pyrrolidine-1-carboxylate (280 mg, 0.5354 mmol) and DIPEA (700 μL, 4.019 mmol) in DCM (3.0 mL) was prepared. The crude acyl chloride generated above was dissolved in DCM (1.5 mL) and added to the sulfonamide solution. The resulting mixture was stirred at room temperature for 16 h. It was then quenched with 0.5 N HCl (10 mL) and extracted with EtOAc (3×10 mL). The combined organic extracts were washed with H₂O (10 mL) and brine (10 mL), then dried over Na₂SO₄, filtered, and evaporated in vacuo. Purification by silica gel chromatography using a gradient eluent of 0 to 100% EtOAc in hexanes gave impure (˜60% pure) product as a pink foam: tert-butyl (4S)-4-[3-[[6-[(6-tert-butyl-2-fluoro-pyridine-3-carbonyl)sulfamoyl]-2-pyridyl]amino]-4-(2-pyridyl)butyl]-2,2-dimethyl-pyrrolidine-1-carboxylate (246.7 mg, 43%); ESI-MS m/z calc. 682.33124, found 683.3 (M+1)⁺; Retention time: 1.71 minutes (LC method A).

### Step 5: (14S)-8-tert-Butyl-12,12-dimethyl-17-[(pyridin-2-yl)methyl]-2λ⁶-thia-3,9,11,18,23-pentaazatetracyclo[17.3.1.111,14.05,10]tetracosa-1(23),5(10),6,8,19,21-hexaene-2,2,4-trione (Compound 407), (14S)-8-tert-butyl-12,12-dimethyl-17-[(pyridin-2-yl)methyl]-2λ⁶-thia-3,9,11,18,23-pentaazatetracyclo[17.3.1.111,14.05,10]tetracosa-1(23),5(10),6,8,19,21-hexaene-2,2,4-trione (Compound 408)

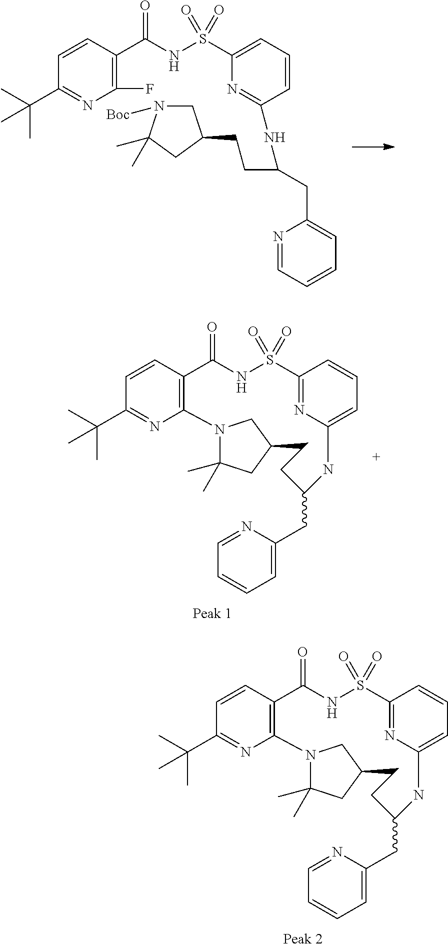 [SMILES: CC(C)(C)OC(=O)N1C[C@@H](CCC(Cc2ccccn2)Nc2cccc(S(=O)(=O)NC(=O)c3ccc(C(C)(C)C)nc3F)n2)CC1(C)C ; CC(C)(C)c1ccc2c(n1)N1C[C@@H](CCC(Cc3ccccn3)Nc3cccc(n3)S(=O)(=O)NC2=O)CC1(C)C ; CC(C)(C)c1ccc2c(n1)N1C[C@@H](CCC(Cc3ccccn3)Nc3cccc(n3)S(=O)(=O)NC2=O)CC1(C)C]

Stage 1: In a 20-mL vial, tert-butyl (4S)-4-[3-[[6-[(6-tert-butyl-2-fluoro-pyridine-3-carbonyl)sulfamoyl]-2-pyridyl]amino]-4-(2-pyridyl)butyl]-2,2-dimethyl-pyrrolidine-1-carboxylate (246.7 mg, 0.2312 mmol) was dissolved in DCM (4 mL). TFA (500 μL, 6.490 mmol) was added, and the resulting solution was stirred at room temperature for 21 h. The mixture was then evaporated in vacuo, diluted with dioxane, and evaporated in vacuo again. This gave a brown oil, 600 mg (>100% yield).

Stage 2: In a 20-mL microwave vial, the crude product from Stage 1 was dissolved in NMP (4 mL), to which K₂CO₃ (396.2 mg, 2.867 mmol) was added. The resulting mixture was flushed with nitrogen, then stirred at 150° C. for 21 h. After cooling to room temperature, the resulting mixture was quenched with 1 N HCl (5 mL), then extracted with EtOAc (3×5 mL). The combined organic extracts were washed with H₂O (2×5 mL) and brine (5 mL), then dried over Na₂SO₄, filtered, and evaporated in vacuo to give a light brown oil. Purification by reverse-phase preparative chromatography using a C₁₈ column and a gradient eluent of 1 to 70% MeCN in H₂O containing 5 mM HCl solution gave 2 products. The first eluted product (“Peak 1”) was pure. The second eluted product (“Peak 2”) was slightly impure (˜90% purity) and was therefore re-purified by reverse-phase preparative chromatography.

“Peak 1”, earlier retention time on reverse-phase UPLC, (14S)-8-tert-butyl-12,12-dimethyl-17-[(pyridin-2-yl)methyl]-2λ⁶-thia-3,9,11,18,23-pentaazatetracyclo[17.3.1.111,14.05,10]tetracosa-1(23),5(10),6,8,19,21-hexaene-2,2,4-trione (15.0 mg, 12%); ¹H NMR (400 MHz, DMSO-d₆) δ 12.63-12.11 (bs, 1H), 8.76 (d, J=5.8 Hz, 1H), 8.45-8.33 (m, 1H), 7.85 (t, J=6.8 Hz, 1H), 7.83-7.71 (m, 1H), 7.56 (t, J=7.9 Hz, 1H), 7.51-7.43 (m, 1H), 7.43-7.33 (m, 1H), 7.24-7.09 (m, 1H), 6.65 (d, J=3.6 Hz, 1H), 6.63 (d, J=3.1 Hz, 1H), 3.46-3.31 (m, 1H), 3.22-3.00 (m, 1H), 3.00-2.75 (m, 1H), 2.32-2.20 (m, 1H), 1.84 (dd, J=11.8, 5.8 Hz, 1H), 1.75-1.65 (m, 2H), 1.58 (s, 3H), 1.53 (s, 3H), 1.52-1.45 (m, 1H), 1.39-1.28 (m, 1H), 1.27 (s, 9H), 1.26-1.15 (m, 1H). [Note: 2H is missing from the overall count of 38 from the product (C₃₀H₃₈N₆O₃S), most likely hidden under the broad water signal.] ESI-MS m/z calc. 562.2726, found 563.2 (M+1)⁺; Retention time: 1.47 minutes (LC method A).

“Peak 2”, later retention time on reverse-phase UPLC, (14S)-8-tert-butyl-12,12-dimethyl-17-[(pyridin-2-yl)methyl]-2λ⁶-thia-3,9,11,18,23-pentaazatetracyclo[17.3.1.111,14.05,10]tetracosa-1(23),5(10),6,8,19,21-hexaene-2,2,4-trione (11.0 mg, 8%); ¹H NMR (400 MHz, DMSO-d₆) δ 12.39-11.85 (bs, 1H), 8.60 (dd, J=5.9, 1.7 Hz, 1H), 8.28 (td, J=7.8, 1.6 Hz, 1H), 7.87 (d, J=8.0 Hz, 1H), 7.77-7.65 (m, 2H), 7.46 (dd, J=8.4, 7.2 Hz, 1H), 7.16 (d, J=8.9 Hz, 1H), 6.88 (d, J=7.1 Hz, 1H), 6.68 (d, J=7.9 Hz, 1H), 6.58 (d, J=8.4 Hz, 1H), 4.83-4.67 (m, 1H), 3.47-3.37 (m, 2H), 3.35 (dd, J=13.5, 3.6 Hz, 1H), 2.93 (dd, J=13.4, 10.5 Hz, 1H), 2.59-2.50 (m, 1H), 2.20-2.06 (m, 1H), 1.81 (dd, J=11.4, 5.1 Hz, 1H), 1.78-1.71 (m, 1H), 1.71-1.66 (m, 1H), 1.65 (s, 3H), 1.56-1.48 (m, 1H), 1.48 (s, 3H), 1.29 (s, 9H), 1.28-1.21 (m, 1H); ESI-MS m/z calc. 562.2726, found 563.2 (M+1)⁺; Retention time: 1.64 minutes (LC method A).

### Example 146: Preparation of (20R)-4-tert-Butyl-17-(4-tert-butylpyridin-2-yl)-10λ⁶-thia-1,3,9,16,25-pentaazatetracyclo[18.3.1.111,15.02,7]pentacosa-2,4,6,11,13,15(25)-hexaene-8,10,10-trione (Compound 409)

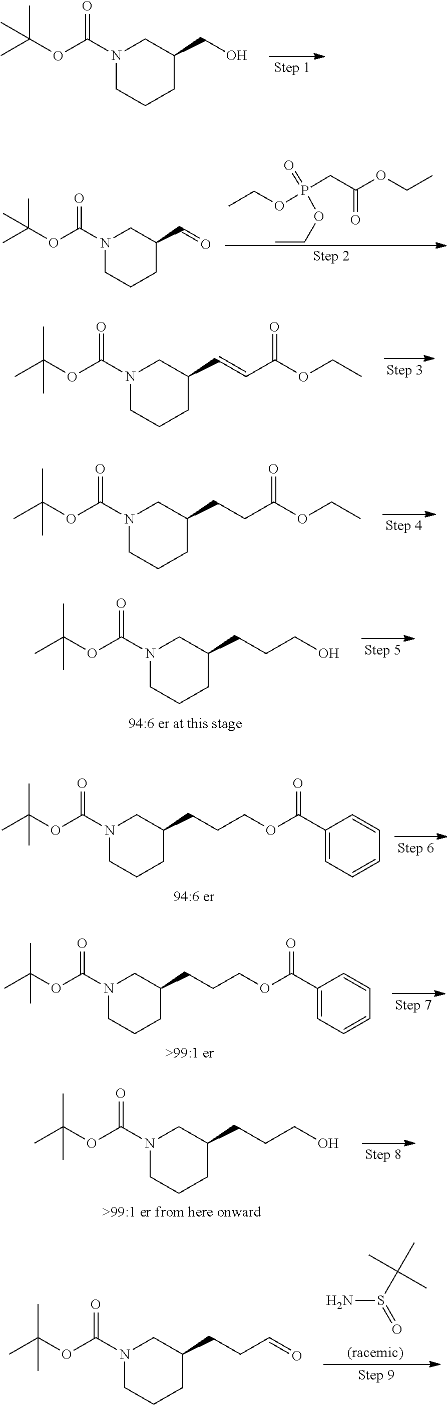

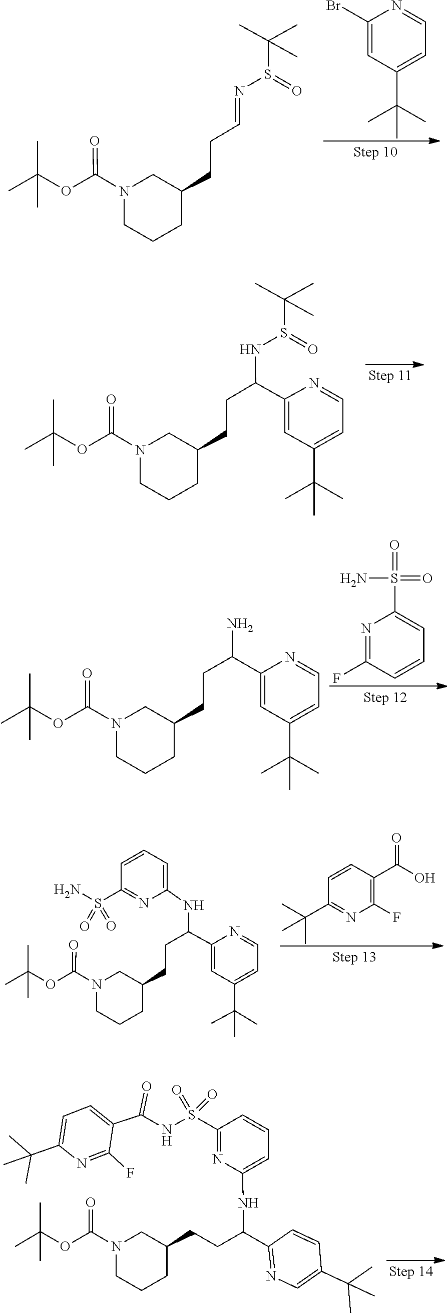

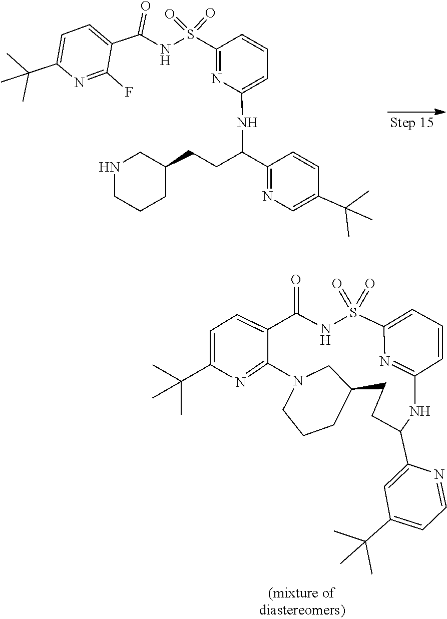

### Step 1: tert-Butyl (3S)-3-formylpiperidine-1-carboxylate

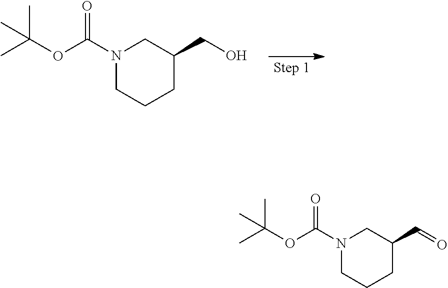

To a three-neck, 100-mL flask equipped with a temperature probe and an addition funnel was charged with a solution of tert-butyl (3S)-3-(hydroxymethyl)piperidine-1-carboxylate (10 g, 46.449 mmol) in dichloromethane (80 mL), and the mixture was cooled to 0° C. A solution of sodium bromide (500 mg, 4.8594 mmol) and sodium bicarbonate (600 mg, 7.1423 mmol) was dissolved in water (20 mL) and was added to the reaction flask. After 15 min of stirring at 0° C., TEMPO (75 mg, 0.4800 mmol) was added, followed by slow addition of sodium hypochlorite in water (50 mL of 1.1 M, 55.000 mmol) dropwise for 1 h (keeping the internal temperature in the 6-8° C. range). The mixture was stirred 10 min at 0° C. and then EtOH (10 mL) was added. After 2 min, the layers were separated at 0° C. (by stopping the stirring). The organic layer was separated and the aqueous layer was extracted with dichloromethane (3×500 mL). The combined organic layers were washed with brine (3×50 mL), dried over Na₂SO₄, filtered, and concentrated to yield crude tert-butyl (3S)-3-formylpiperidine-1-carboxylate (9.89 g, 95%) as a pale orange oil. ¹H NMR (400 MHz, CDCl₃) δ 9.70 (s, 1H), 3.92 (br. s., 1H), 3.72-3.58 (m, 1H), 3.33 (dd, J=13.7, 8.3 Hz, 1H), 3.09 (ddd, J=13.1, 9.4, 3.2 Hz, 1H), 2.43 (br. s., 1H), 1.95 (br. s., 1H), 1.83-1.72 (m, 1H), 1.71-1.62 (m, 2H), 1.47 (s, 9H). ESI-MS m/z calc. 213.1365, found 158.2 (M-tBu+2H=M−55)⁺; Retention time: 1.68 minutes (LC method E).

### Step 2: tert-Butyl (3R)-3-[(E)-3-ethoxy-3-oxo-prop-1-enyl]piperidine-1-carboxylate

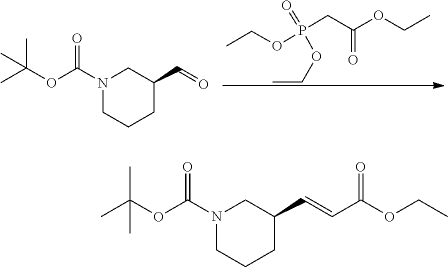

A flame-dried round-bottomed flask was charged with ethyl 2-diethoxyphosphorylacetate (9.3790 g, 8.3 mL, 41.835 mmol) and THF (130 mL). The solution was cooled to 78° C. and then a solution of NaHMDS in THF (42 mL of 1 M, 42.000 mmol) was added. The mixture was stirred at 78° C. for 30 min. Then, this mixture was added to a cooled (to 78° C.) solution of tert-butyl (3S)-3-formylpiperidine-1-carboxylate (9.85 g, 43.876 mmol) in THF (45 mL). The mixture was stirred at 78° C. for 30 min. The reaction was quenched through the addition of a saturated aqueous solution of NH₄Cl (50 mL). The layers were separated and the aqueous phase was extracted EtOAc (3×50 mL). The combined organic phases were washed with brine (50 mL) and dried over anhydrous sodium sulfate, filtered and concentrated under reduced pressure. The crude material was purified through silica gel column chromatography using a gradient of 0 to 50% EtOAc/heptanes to afford tert-butyl (3R)-3-[(E)-3-ethoxy-3-oxo-prop-1-enyl]piperidine-1-carboxylate (8.3095 g, 67%) as a clear oil. ESI-MS m/z calc. 283.1784, found 228.2 (M-tBu+2H=M−55)⁺; Retention time: 1.93 minutes (LC method E).

### Step 3: tert-Butyl (3R)-3-(3-ethoxy-3-oxo-propyl)piperidine-1-carboxylate

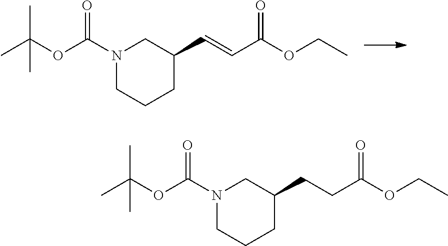

5% Palladium on carbon (50% wet, 5% w/w, 650 mg, 0.3054 mmol) was added to a mixture of tert-butyl(3R)-3-[(E)-3-ethoxy-3-oxo-prop-1-enyl]piperidine-1-carboxylate (8.3 g, 29.291 mmol) in methanol (100 mL). Hydrogen was bubbled into the suspension for 2 min and then the reaction mixture was stirred under hydrogen atmosphere for 18 h. The reaction mixture was filtered on Celite and rinsed with methanol. The filtrate was concentrated in vacuo to give crude tert-butyl (3R)-3-(3-ethoxy-3-oxo-propyl)piperidine-1-carboxylate (8.104 g, 97%) as a colorless oil. ESI-MS m/z calc. 285.194, found 230.2 (M-tBu+2H=M−55)⁺; Retention time: 1.92 minutes (LC method E).

### Step 4: tert-Butyl (3R)-3-(3-hydroxypropyl)piperidine-1-carboxylate

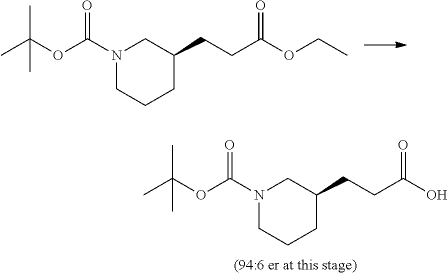

To a solution of tert-butyl (3R)-3-(3-ethoxy-3-oxo-propyl)piperidine-1-carboxylate (8.1 g, 27.475 mmol) in THF (100 mL) at 0° C. was added LiBH₄ (1.8 g, 82.630 mmol). The reaction mixture was stirred at room temperature for 16 h. A saturated aqueous solution of NH₄Cl (50 mL) was added slowly to the reaction mixture at 0° C. for 15 min. The product was extracted with EtOAc (3×50 mL). The combined organic layers were washed with brine (50 mL), dried over anhydrous sodium sulfate, filtered and concentrated in vacuo to afford crude tert-butyl (3R)-3-(3-hydroxypropyl)piperidine-1-carboxylate (6.65 g, 99%) as a colorless oil. ESI-MS m/z calc. 243.1834, found 188.4 (M−tBu+2H=M−55)⁺; Retention time: 1.69 minutes (LC method E).

### Step 5: tert-Butyl (3R)-3-(3-benzoyloxypropyl)piperidine-1-carboxylate

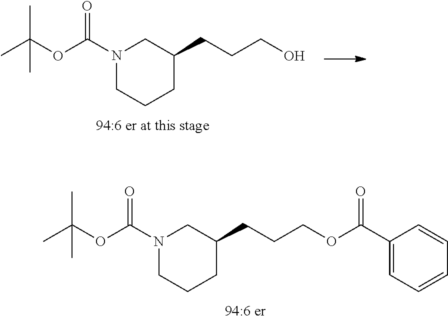 [SMILES: CC(C)(C)OC(=O)N1CCC[C@H](CCCO)C1 ; CC(C)(C)OC(=O)N1CCC[C@H](CCCOC(=O)c2ccccc2)C1]

To a solution of tert-butyl (3R)-3-(3-hydroxypropyl)piperidine-1-carboxylate (6.15 g, 24.464 mmol) in pyridine (58.680 g, 60 mL, 741.85 mmol) at 0° C. was added benzoyl chloride (6.8970 g, 5.7 mL, 49.066 mmol). The reaction mixture was then warmed to room temperature and stirred at room temperature for 2 h. DCM (50 mL) was added and then a saturated aqueous solution of NH₄Cl (50 mL) was added slowly to the reaction mixture at 0° C. over 5 min. The product was extracted with DCM (3×50 mL) and the combined organic layers were washed with brine (60 mL), dried over anhydrous sodium sulfate, filtered and concentrated in vacuo. The crude residue was purified by silica gel chromatography using a gradient of EtOAc in heptanes to give tert-butyl (3R)-3-(3-benzoyloxypropyl)piperidine-1-carboxylate (8.25 g, 94%) as a colorless oil. ESI-MS m/z calc. 347.2097, found 248.2 (M-Boc+2H=M−99)⁺; Retention time: 5.05 minutes (LC method F).

### Step 6: tert-Butyl (3R)-3-(3-benzoyloxypropyl)piperidine-1-carboxylate

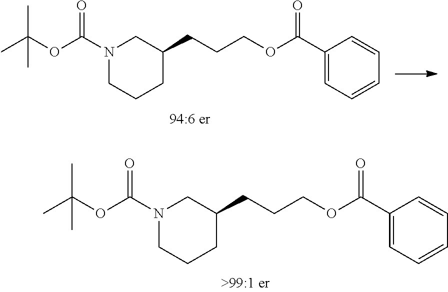 [SMILES: CC(C)(C)OC(=O)N1CCC[C@H](CCCOC(=O)c2ccccc2)C1 ; CC(C)(C)OC(=O)N1CCC[C@H](CCCOC(=O)c2ccccc2)C1]

A 94:6 er mixture of tert-butyl 3-(3-benzoyloxypropyl)piperidine-1-carboxylate (8.05 g, 22.335 mmol) was purified by chiral SFC (using a LUX-Cellulose-5 column, and 20% methanol in 100 bar CO₂ as the eluent) to afford tert-butyl (3R)-3-(3-benzoyloxypropyl)piperidine-1-carboxylate (5.995 g, 77%); ESI-MS m/z calc. 347.2097, found 248.2 (M-Boc+2H=M−99)⁺; Retention time: 2.15 minutes (LC method E). Minor product: tert-butyl (3S)-3-(3-benzoyloxypropyl)piperidine-1-carboxylate (501.7 mg, 5%); ESI-MS m/z calc. 347.2097, found 248.2 (M-Boc+2H=M−99)⁺; Retention time: 2.14 minutes (LC method E).

### Step 7: tert-Butyl (3R)-3-(3-hydroxypropyl)piperidine-1-carboxylate

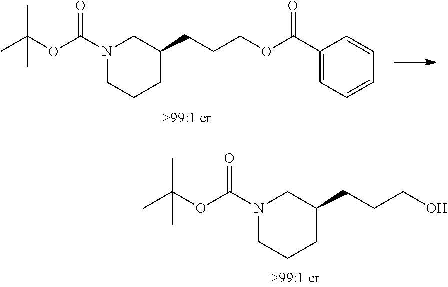 [SMILES: CC(C)(C)OC(=O)N1CCC[C@H](CCCOC(=O)c2ccccc2)C1 ; CC(C)(C)OC(=O)N1CCC[C@H](CCCO)C1]

A solution of sodium methoxide in MeOH (3.8 mL of 25% w/v, 17.585 mmol) was added dropwise to a solution of tert-butyl (3R)-3-(3-benzoyloxypropyl)piperidine-1-carboxylate (6 g, 17.148 mmol) in methanol (75 mL) at room temperature. The mixture was stirred at room temperature for 24 h. The mixture was concentrated in vacuo and the crude material was then purified by silica gel column chromatography using a gradient of 0 to 100% EtOAc in heptanes to afford tert-butyl (3R)-3-(3-hydroxypropyl)piperidine-1-carboxylate (3.96 g, 94%) as a clear oil. ESI-MS m/z calc. 243.1834, found 188.2 (M tBu+2H=M−55)⁺; Retention time: 1.68 minutes (LC method E).

### Step 8: tert-Butyl (3R)-3-(3-oxopropyl)piperidine-1-carboxylate

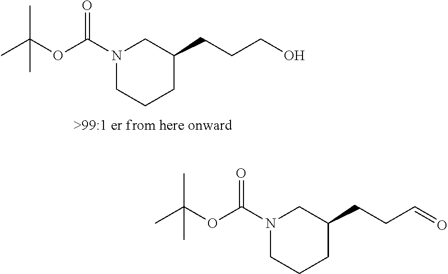 [SMILES: CC(C)(C)OC(=O)N1CCC[C@H](CCCO)C1 ; CC(C)(C)OC(=O)N1CCC[C@H](CCC=O)C1]

To a three-neck, 300-mL flask equipped with a temperature probe and an addition funnel was charged with a solution of tert-butyl (3R)-3-(3-hydroxypropyl)piperidine-1-carboxylate (4 g, 16.323 mmol) in DCM (32 mL), and the mixture was cooled to 0° C. A solution of sodium bromide (175 mg, 1.7008 mmol) and sodium bicarbonate (210 mg, 2.4998 mmol) dissolved in water (8 mL) was added. After 15 min of stirring at 0° C., TEMPO (30 mg, 0.1920 mmol) was added, followed by slow addition of sodium hypochlorite in water (17.5 mL of 1.1 M, 19.250 mmol) for 30 min (dropwise keeping the internal temperature in the 6-8° C. range). Ethanol (10 mL) was added and stirring was stopped until the layers were separated. The organic layer was then separated and the aqueous layer was extracted with DCM (3×20 mL). The combined organic layers were washed with brine (3×40 mL), dried over Na₂SO₄, filtered, and concentrated to yield crude tert-butyl (3R)-3-(3-oxopropyl)piperidine-1-carboxylate (3.41 g, 83%) as an orange oil. ¹H NMR (400 MHz, CDCl₃) δ 9.79 (s, 1H), 4.10-3.68 (m, 2H), 2.80 (t, J=11.4 Hz, 1H), 2.57 (s, 1H), 2.50 (t, J=7.3 Hz, 2H), 1.89-1.76 (m, 1H), 1.46 (s, 14H), 1.20-1.01 (m, 1H). ESI-MS m/z calc. 241.1678, found 186.2 (M-tBu+2H=M−55)⁺; Retention time: 1.77 minutes (LC method E).

### Step 9: tert-Butyl (3R)-3-[(3E)-3-tert-butylsulfinyliminopropyl]piperidine-1-carboxylate

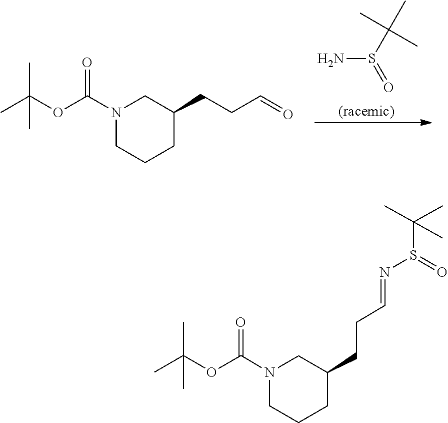

To tert-butyl (3R)-3-(3-oxopropyl)piperidine-1-carboxylate (3 g, 11.810 mmol) dissolved in DCM (35 mL) was added racemic 2-methylpropane-2-sulfinamide (2.15 g, 17.739 mmol), magnesium sulfate (8 g, 66.463 mmol) and pyridinium p-toluenesulfonate (300 mg, 1.1890 mmol). The reaction mixture was stirred at room temperature for 18 h. The reaction mixture was filtered then concentrated. The crude material was purified by silica gel chromatography eluted with a gradient of 20 to 100% EtOAc in heptane to afford tert-butyl (3R)-3-[(3E)-3-tert-butylsulfinyliminopropyl]piperidine-1-carboxylate (3.75 g, 92%) as a yellow oil. ESI-MS m/z calc. 344.2134, found 245.2 (M-Boc+2H=M−99)⁺; Retention time: 1.94 minutes (LC method E).

### Step 10: tert-Butyl (3R)-3-[3-(4-tert-butyl-2-pyridyl)-3-(tert-butylsulfinylamino)propyl]piperidine-1-carboxylate

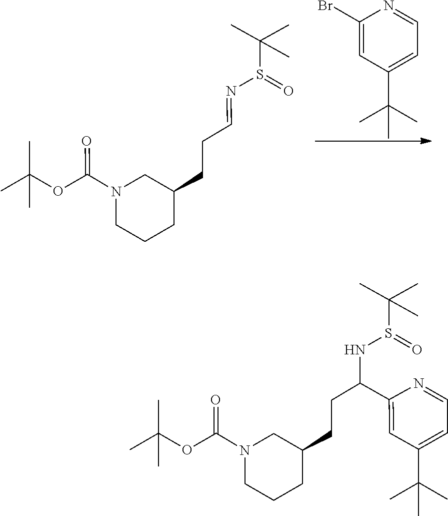

A solution of n-butyllithium in hexanes (3.9 mL of 2.5 M, 9.7500 mmol) was added dropwise to a solution of 2-bromo-4-tert-butyl-pyridine (2.1 g, 8.9158 mmol) in THF (45 mL) at 78° C. The mixture was stirred at 78° C. for 45 min. A solution of tert-butyl (3R)-3-[(3E)-3-tert-butylsulfinyliminopropyl]piperidine-1-carboxylate (2.75 g, 7.9743 mmol) in THF (15 mL) was added dropwise. The reaction was slowly warmed to room temperature over 4 h. A saturated aqueous solution of NH₄Cl (50 mL) was added and the product was extracted with EtOAc (3×50 mL). The combined organic phases were washed with brine (50 mL), dried over Na₂SO₄, filtrated and evaporated in vacuo. The crude product was purified by reverse phase chromatography eluting with a gradient of 5% to 100% MeOH in water (with 0.1% formic acid as a modifier) to give tert-butyl (3R)-3-[3-(4-tert-butyl-2-pyridyl)-3-(tert-butylsulfinylamino)propyl]piperidine-1-carboxylate (2.38 g, 57%) as a yellow oil. ESI-MS m/z calc. 479.3182, found 480.4 (M+1)⁺; Retention time: 1.82 minutes (LC method E).

### Step 11: tert-Butyl (3R)-3-[3-amino-3-(4-tert-butyl-2-pyridyl)propyl]piperidine-1-carboxylate

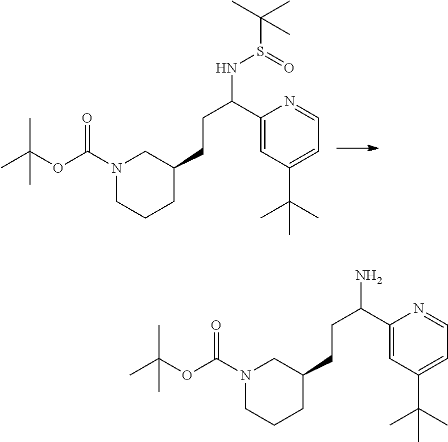

To a solution of tert-butyl (3R)-3-[3-(4-tert-butyl-2-pyridyl)-3-(tert-butylsulfinylamino)propyl]piperidine-1-carboxylate (2.38 g, 4.5296 mmol) in THF (16 mL) and water (4 mL) was added iodine (650 mg, 2.5610 mmol) and the mixture was stirred at 60° C. for 2 h. The reaction mixture was cooled to room temperature and extracted with EtOAc (3×20 mL). The combined organic layers were washed with saturated aqueous NaHCO₃(40 mL), 10% aqueous sodium thiosulfate (40 mL) and brine (40 mL), dried over sodium sulfate, filtered and concentrated under reduced pressure to afford tert-butyl (3R)-3-[3-amino-3-(4-tert-butyl-2-pyridyl)propyl]piperidine-1-carboxylate (2.04 g, 56%) as a yellow oil. ESI-MS m/z calc. 375.2886, found 376.4 (M+1)⁺; Retention time: 1.48 minutes (LC method E).

### Step 12: tert-Butyl (3R)-3-[3-(4-tert-butyl-2-pyridyl)-3-[(6-sulfamoyl-2-pyridyl)amino]propyl]piperidine-1-carboxylate

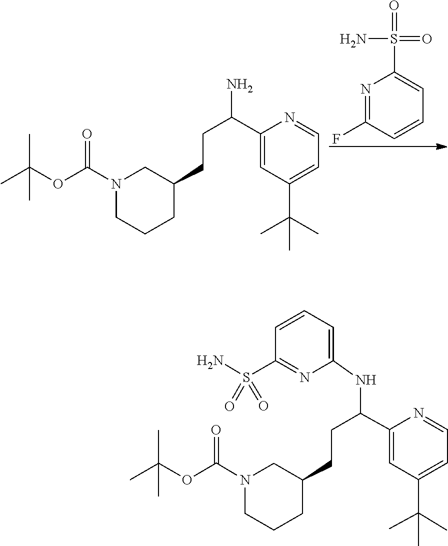

To a solution of tert-butyl (3R)-3-[3-amino-3-(4-tert-butyl-2-pyridyl)propyl]piperidine-1-carboxylate (2 g, 2.3060 mmol) and 6-fluoropyridine-2-sulfonamide (609.37 mg, 3.4590 mmol) in acetonitrile (20 mL) was added DIPEA (983.51 mg, 1.3255 mL, 7.6098 mmol). The reaction tube was sealed and the mixture was stirred at 120° C. for 24 h. The reaction mixture was then cooled to room temperature and was partitioned between ethyl acetate (50 mL) and water (50 mL). The aqueous phase was extracted with ethyl acetate (2×20 mL). The combined organic layers were washed with brine (20 mL), dried over sodium sulfate, filtered and concentrated in vacuo. The crude product was purified by reverse phase chromatography, eluting with a gradient of 5% to 100% MeOH/water (with 0.1% formic acid as a modifier). The obtained fractions were evaporated in vacuo, and the resulting aqueous solution was neutralized with sat. aqueous NaHCO₃(20 mL). The resulting aqueous solution was extracted with DCM (4×20 mL) and the combined organic layers were washed with brine, dried over sodium sulfate, filtered and concentrated under reduced pressure. This gave tert-butyl (3R)-3-[3-(4-tert-butyl-2-pyridyl)-3-[(6-sulfamoyl-2-pyridyl)amino]propyl]piperidine-1-carboxylate (210 mg, 17%) as a yellow semi-solid. ESI-MS m/z calc. 531.2879, found 532.4 (M+1)⁺; Retention time: 1.59 minutes (LC method E).

### Step 13: tert-Butyl (3R)-3-[3-[[6-[(6-tert-butyl-2-fluoro-pyridine-3-carbonyl)sulfamoyl]-2-pyridyl]amino]-3-(5-tert-butyl-2-pyridyl)propyl]piperidine-1-carboxylate

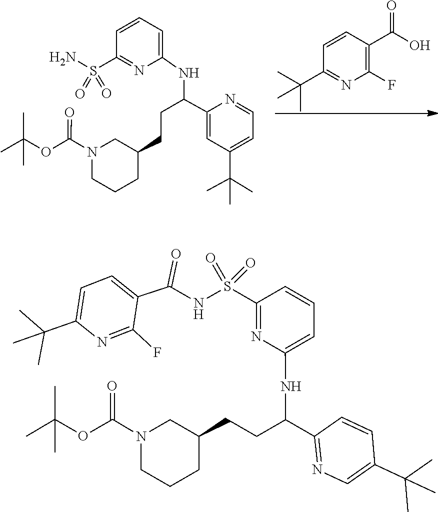

A round-bottomed flask was charged with 6-tert-butyl-2-fluoro-pyridine-3-carboxylic acid (112 mg, 0.5679 mmol) in THF (9 mL). 1,1′-Carbonyldiimidazole (200 mg, 1.2334 mmol) was added and the mixture was stirred at room temperature for 2 h. In a separate flask, a solution of tert-butyl (3R)-3-[3-(5-tert-butyl-2-pyridyl)-3-[(6-sulfamoyl-2-pyridyl)amino]propyl]piperidine-1-carboxylate (300 mg, 0.5637 mmol) in THF (5 mL) was prepared and it was added into the activated acid solution. 1,8-Diazabicyclo[5.4.0]undec-7-ene (458.10 mg, 0.45 mL, 3.0091 mmol) was then added and the reaction mixture was stirred at room temperature for 21 h. Then, the solvents were removed under reduced pressure and the resulting thick oil was treated with EtOAc (10 mL) and water (10 mL). The aqueous phase was extracted with EtOAc (3×10 mL). The combined organic layers were washed with aqueous solution of 1 N HCl (20 mL), brine (20 mL), dried over sodium sulfate, filtered and concentrated in vacuo. The crude mixture was purified by reverse phase chromatography, eluting with a gradient of 5% to 100% MeOH in water (with 0.1% of formic acid as a modifier) to afford tert-butyl (3R)-3-[3-[[6-[(6-tert-butyl-2-fluoro-pyridine-3-carbonyl)sulfamoyl]-2-pyridyl]amino]-3-(5-tert-butyl-2-pyridyl)propyl]piperidine-1-carboxylate (331.2 mg, 74%) as a pale yellow oil. ¹H NMR (400 MHz, DMSO-d₆) δ 8.38 (d, J=5.4 Hz, 1H), 8.13 (t, J=8.8 Hz, 1H), 7.47-7.40 (m, 1H), 7.40-7.37 (m, 1H), 7.27 (d, J=5.4 Hz, 1H), 7.19 (d, J=5.4 Hz, 1H), 7.09 (d, J=8.3 Hz, 1H), 6.99 (d, J=7.3 Hz, 1H), 6.51 (d, J=8.3 Hz, 1H), 5.00-4.77 (br. s., 1H), 3.74-3.64 (m, 1H), 2.76-2.59 (m, 1H), 1.89-1.73 (m, 2H), 1.68-1.57 (m, 1H), 1.54-1.42 (m, 1H), 1.39-1.31 (m, 9H), 1.27 (s, 9H), 1.20-1.15 (m, 17H). ¹⁹F NMR (377 MHz, DMSO-d₆) δ −65.98 (s, 1F). ESI-MS m/z calc. 710.3626, found 711.4 (M+1)⁺; Retention time: 1.93 minutes (LC method E).

### Step 14: 6-tert-Butyl-N-[[6-[[1-(4-tert-butyl-2-pyridyl)-3-[(3R)-3-piperidyl]propyl]amino]-2-pyridyl]sulfonyl]-2-fluoro-pyridine-3-carboxamide

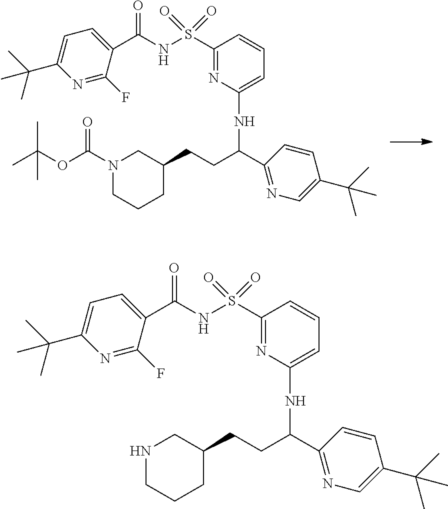

In a round-bottomed flask, to a solution of tert-butyl (3R)-3-[3-[[6-[(6-tert-butyl-2-fluoro-pyridine-3-carbonyl)sulfamoyl]-2-pyridyl]amino]-3-(4-tert-butyl-2-pyridyl)propyl]piperidine-1-carboxylate (325 mg, 0.4567 mmol) in DCM (6.5 mL), 4 M HCl in dioxane (1.75 mL of 4 M, 7.00 mmol) was added and stirred for 18 h. The solvent was evaporated to afford a mixture of two diastereomers, 6-tert-butyl-N-[[6-[[1-(4-tert-butyl-2-pyridyl)-3-[(3R)-3-piperidyl]propyl]amino]-2-pyridyl]sulfonyl]-2-fluoro-pyridine-3-carboxamide (hydrochloride salt) (325.1 mg, 99%) as a yellow solid. ESI-MS m/z calc. 610.3101, found 611.2 (M+1)⁺; Retention time: 1.4 minutes (LC method E).

### Step 15: (20R)-4-tert-Butyl-17-(4-tert-butylpyridin-2-yl)-10λ⁶-thia-1,3,9,16,25-pentaazatetracyclo[18.3.1.111,15.02,7]pentacosa-2,4,6,11,13,15(25)-hexaene-8,10,10-trione (Compound 409)

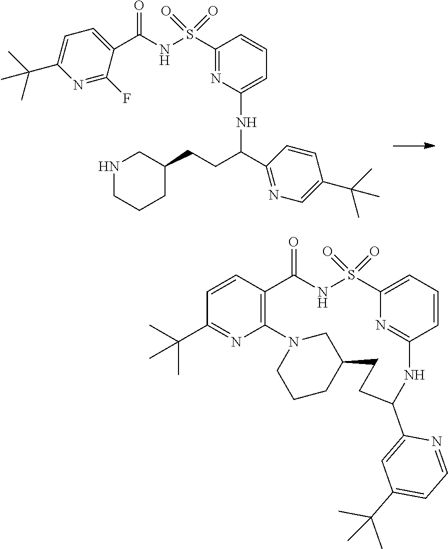

6-tert-Butyl-N-[[6-[[1-(4-tert-butyl-2-pyridyl)-3-[(3R)-3-piperidyl]propyl]amino]-2-pyridyl]sulfonyl]-2-fluoro-pyridine-3-carboxamide (hydrochloride salt) (325 mg, 0.4508 mmol) was dissolved in DMSO (6.5 mL) and potassium carbonate (815 mg, 5.8970 mmol) was added. The reaction tube was sealed and the mixture was heated at 120° C. for 24 h. The mixture was then cooled to room temperature and the crude mixture was directly purified by reverse phase chromatography using a gradient of 5% to 100% MeOH in water (with 0.1% formic acid as a modifier) to afford (20R)-4-tert-butyl-17-(4-tert-butylpyridin-2-yl)-1W-thia-1,3,9,16,25-pentaazatetracyclo[18.3.1.111,15.02,7]pentacosa-2,4,6,11,13,15(25)-hexaene-8,10,10-trione (46.3 mg, 17%) as a beige solid. ¹H NMR (400 MHz, DMSO-d₆) δ 8.41 (d, J=5.4 Hz, 1H), 7.56-7.45 (m, 2H), 7.44-7.37 (m, 1H), 7.24 (d, J=4.9 Hz, 1H), 7.09-6.97 (m, 1H), 6.96-6.84 (m, 1H), 6.68-6.57 (m, 1H), 6.57-6.44 (m, 1H), 4.81-4.67 (m, 1H), 4.63-4.45 (m, 1H), 4.17-3.98 (m, 1H), 2.79-2.55 (m, 1H), 2.45-2.35 (m, 1H), 1.81-1.69 (m, 1H), 1.56 (br. s., 3H), 1.33-1.05 (m, 22H), 1.00-0.88 (m, 1H). ESI-MS m/z calc. 590.3039, found 591.4 (M+1)⁺; Retention time: 4.46 minutes (LC method F).

### Example 147: Preparation of (14S)-8-tert-Butyl-17-{4-[(2-methoxyethyl) (methyl)amino] pyridin-2-yl}-12,12-dimethyl-2λ⁶-thia-3,9,11,18,23-pentaaza tetracyclo[17.3.1.111,14.05,10]tetracosa-1(23),5(10),6,8,19,21-hexaene-2,2,4-trione (Compound 410)

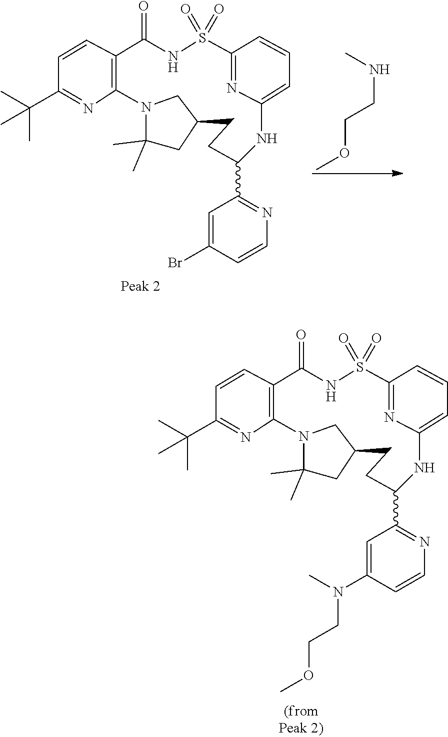

In a 1-dram vial, Peak 2 of (14S)-17-(4-bromopyridin-2-yl)-8-tert-butyl-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (20 mg, 0.03187 mmol) and 2-methoxy-N-methyl-ethanamine (33.2 mg, 0.3725 mmol) were mixed with dioxane (300 μL), to which RuPhos Pd G3 (2.5 mg, 0.002989 mmol) and sodium tert-butoxide (12.9 mg, 0.1342 mmol) were added. The resulting mixture was sparged with nitrogen gas, then stirred at 110° C. for 18 h. It was then cooled to room temperature, diluted with MeOH (700 μL), filtered, and purified by reverse-phase preparative chromatography using a C₁₈ column and a gradient eluent of 1 to 70% MeCN in H₂O containing 5 mM HCl solution to give (14S)-8-tert-butyl-17-{4-[(2-methoxy ethyl)(methyl)amino]pyri din-2-yl}-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (3.7 mg, 17%); ¹H NMR (400 MHz, DMSO-d₆) δ 13.62 and 13.55 (two singlets combining for one proton, 1H), 12.44 (s, 1H), 8.25-8.06 (m, 1H), 7.93 (d, J=8.7 Hz, 1H), 7.74 (dd, J=8.4, 7.3 Hz, 1H), 7.65 (d, J=7.9 Hz, 1H), 7.22 (d, J=7.2 Hz, 1H), 7.15 and 6.85 (singlet and doublet for a total of 1H), 7.08-6.98 (m, 1H), 6.97 (d, J=8.4 Hz, 1H), 6.65 (d, J=8.0 Hz, 1H), 5.28 (t, J=9.8 Hz, 1H), 3.91-3.62 (m, 2H), 3.59-3.47 (m, 2H), 3.28 (dd, J=9.7, 6.7 Hz, 1H), 3.26-3.06 (four singlets combining for two methyls, 6H), 2.71-2.59 (m, 1H), 2.43-2.29 (m, 1H), 2.05-1.87 (m, 2H), 1.83 (dd, J=11.7, 5.1 Hz, 1H), 1.80-1.71 (m, 1H), 1.68 (s, 3H), 1.55 (t, J=12.5 Hz, 1H), 1.50 (s, 3H), 1.48-1.38 (m, 1H), 1.28 (s, 9H). ESI-MS m/z calc. 635.3254, found 636.3 (M+1)⁺; Retention time: 1.71 minutes (LC method A).

### Example 148: Preparation of (14S,17R)-8-tert-Butyl-17-{6-[(2-methoxyethyl) (methyl)amino]pyridin-2-yl}-12,12-dimethyl-2λ⁶-thia-3,9,11,18,23-pentaaza tetracyclo[17.3.1.111,14.05,10]tetracosa-1(23),5(10),6,8,19,21-hexaene-2,2,4-trione (Compound 411)

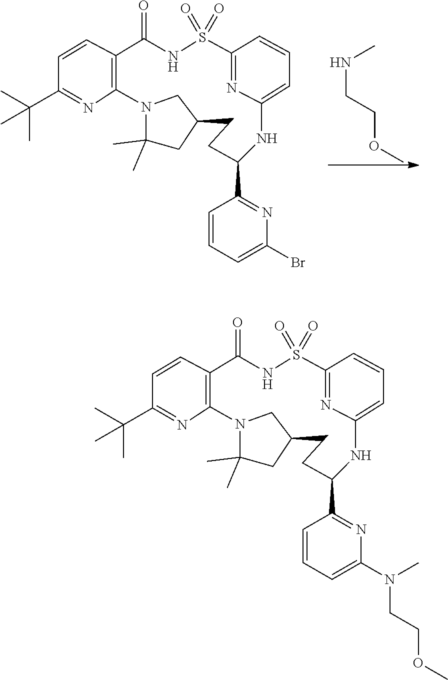

In a 1-dram vial, (14S,17R)-17-(6-bromopyridin-2-yl)-8-tert-butyl-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (20 mg, 0.03187 mmol) and 2-methoxy-N-methyl-ethanamine (33.2 mg, 0.3725 mmol) were mixed with dioxane (300 μL), to which RuPhos Pd G3 (2.5 mg, 0.002989 mmol) and sodium tert-butoxide (12.9 mg, 0.1342 mmol) were added. The resulting mixture was sparged with nitrogen gas, then stirred at 110° C. for 18 h. It was then cooled to room temperature, diluted with MeOH (700 μL), filtered, and purified by reverse-phase preparative chromatography using a C₁₈ column and a gradient eluent of 1 to 70% MeCN in H₂O containing 5 mM HCl solution to give (14S,17R)-8-tert-butyl-17-{6-[(2-methoxyethyl)(methyl)amino]pyridin-2-yl}-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (3.6 mg, 17%); ¹H NMR (400 MHz, DMSO-d₆) δ 12.58-12.29 (bs, 1H), 12.11-11.19 (bs, 1H), 8.25-7.82 (m, 1H), 7.76-7.56 (m, 2H), 7.56-7.26 (m, 1H), 7.22-7.06 (m, 1H), 6.89 (d, J=8.5 Hz, 1H), 6.65 (d, J=8.0 Hz, 1H), 6.59-6.21 (m, 1H), 5.34-4.97 (m, 1H), 3.85-3.67 (m, 2H), 3.53-3.40 (m, 2H), 3.36-2.91 (m, 7H), 2.80-2.64 (m, 1H), 2.30-2.14 (m, 1H), 2.12-1.89 (m, 2H), 1.85 (dd, J=11.7, 5.2 Hz, 1H), 1.80-1.69 (m, 1H), 1.64 (s, 3H), 1.57 (t, J=12.5 Hz, 1H), 1.50 (s, 3H), 1.47-1.38 (m, 1H), 1.28 (s, 9H). ESI-MS m/z calc. 635.3254, found 636.3 (M+1)⁺; Retention time: 1.69 minutes (LC method A).

### Example 149: Preparation of (14S)-8-tert-Butyl-17-{5-[(2-methoxyethyl) (methyl)amino]pyridin-2-yl}-12,12-dimethyl-2λ⁶-thia-3,9,11,18,23-pentaaza tetracyclo[17.3.1.111,14.05,10]tetracosa-1(23),5(10),6,8,19,21-hexaene-2,2,4-trione (Compound 412)

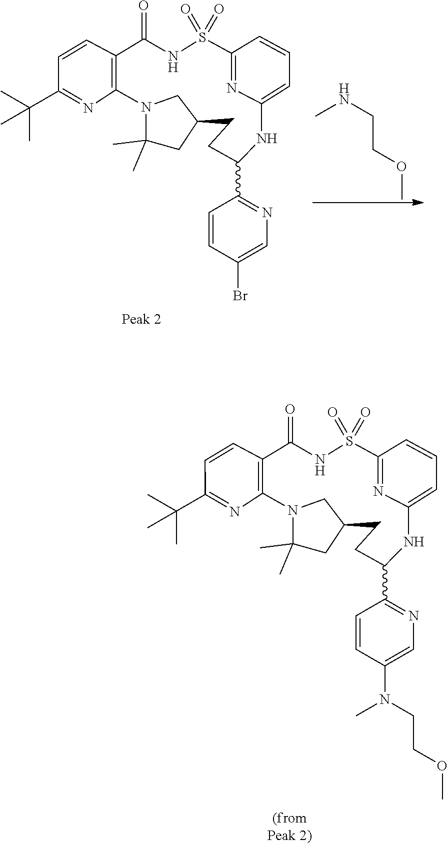

In a 1-dram vial, Peak 2 of (14S)-17-(5-bromopyridin-2-yl)-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (20.0 mg, 0.03187 mmol) and 2-methoxy-N-methyl-ethanamine (20.2 mg, 0.2266 mmol) were mixed with dioxane (300 μL), to which RuPhos Pd G3 (3.1 mg, 0.003706 mmol) and sodium tert-butoxide (16.2 mg, 0.1686 mmol) were added. The resulting mixture was sparged with nitrogen gas, then stirred at 90° C. for 18 h. It was then cooled to room temperature, diluted with MeOH (700 μL), filtered, and purified by reverse-phase preparative chromatography using a C₁₈ column and a gradient eluent of 1 to 70% MeCN in H₂O containing 5 mM HCl solution to give (14S)-8-tert-butyl-17-{5-[(2-methoxy ethyl)(methyl)amino]pyri din-2-yl}-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (5.2 mg, 24%); ¹H NMR (400 MHz, DMSO-d₆) δ 15.77-15.24 (bs, 1H), 12.58-12.16 (bs, 1H), 7.97 (s, 1H), 7.92-7.72 (bs, 2H), 7.72 (t, J=7.8 Hz, 1H), 7.65 (d, J=8.0 Hz, 1H), 7.19 (d, J=7.2 Hz, 1H), 6.93 (d, J=8.5 Hz, 1H), 6.66 (d, J=8.0 Hz, 1H), 5.44-5.27 (m, 1H), 3.68-3.57 (m, 2H), 3.49 (t, J=5.3 Hz, 2H), 3.33-3.24 (m, 1H), 3.21 (s, 3H), 2.99 (s, 3H), 2.72-2.60 (m, 1H), 2.40-2.25 (m, 1H), 1.99-1.87 (m, 2H), 1.84 (dd, J=11.6, 5.2 Hz, 1H), 1.79-1.70 (m, 1H), 1.67 (s, 3H), 1.56 (t, J=12.4 Hz, 1H), 1.50 (s, 3H), 1.48-1.37 (m, 1H), 1.28 (s, 9H), 1.20-1.06 (m, 1H). ESI-MS m/z calc. 635.3254, found 636.3 (M+1)⁺; Retention time: 1.69 minutes (LC method A).

### Example 150: Preparation of (14S)-8-tert-Butyl-17-{5-[1-(3,3-dimethylbutyl)-1H-pyrazol-3-yl]pyridin-2-yl}-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 413)

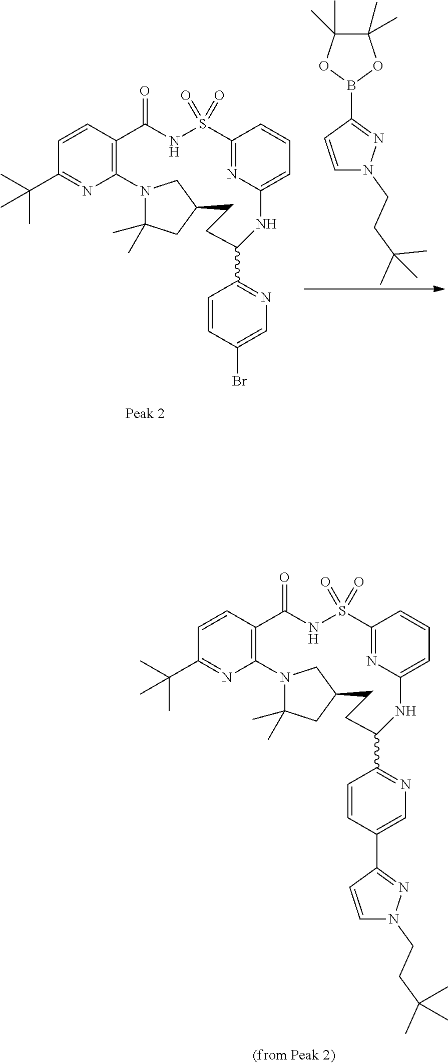

In a 1-dram vial, Peak 2 of (14S)-17-(5-bromopyridin-2-yl)-8-tert-butyl-12,12-dimethyl-2λ⁶-thia-3,9,11,18,23-pentanzatetracyclo[17.3.1.111,14.05,10]tetracos 1(23),5(10),6,8,19,21-hexaene-2,2,4-trione (21.0 mg, 0.03346 mmol) and 1-(3,3-dimethylbutyl)-3-(4,4,5,5-tetramethyl-1,3,2-dioxaborolan-2-yl)pyrazole (22.5 mg, 0.08088 mmol) were mixed with dioxane (500 μL), to which Pd(dppf)Cl₂DCM (3 mg, 0.003674 mmol) and aqueous Na₂CO₃ (80 4 of 2.0 M, 0.1600 mmol) were added. The resulting mixture was sparged with nitrogen gas, then stirred at 120° C. for 110 min. It was then cooled to room temperature, diluted with MeOH (600 μL), filtered, and purified by reverse-phase preparative chromatography using a C₁₈ column and a gradient eluent of 1 to 99% MeCN in H₂O containing 5 mM HCl solution to give an off-white solid, (145)-8-tert-butyl-17-{5-[1-(3,3-dimethylbutyl)-1H-pyrazol-3-yl]pyridin-2-yl}-12,12-dimethyl-2λ⁶-thia-3,9,11,18,23-pentaazatetracyclo [17.3.1.111,14.05,10]tetracosa-1(23),5(10),6,8,19,21-hexaene-2,2,4-trione (Hydrochloride salt) (15.0 mg, 61%); ¹H NMR (400 MHz, DMSO-d₆) δ 12.49 (s, 1H), 8.66 (d, J=2.3 Hz, 1H), 7.99-7.89 (m, 1H), 7.79 (d, J=9.0 Hz, 1H), 7.66 (dd, J=8.5, 7.3 Hz, 1H), 7.64 (d, J=8.0 Hz, 2H), 7.52 (d, J=1.8 Hz, 1H), 7.12 (d, J=7.2 Hz, 1H), 6.92 (d, J=8.4 Hz, 1H), 6.66 (d, J=8.0 Hz, 1H), 6.45 (d, J=1.9 Hz, 1H), 5.40-5.30 (m, 1H), 4.15-4.05 (m, 2H), 3.24 (dd, J=10.3, 6.7 Hz, 1H), 2.77 (t, J=10.4 Hz, 1H), 2.32-2.17 (m, 1H), 2.04-1.91 (m, 2H), 1.86 (dd, J=11.7, 5.2 Hz, 1H), 1.81-1.71 (m, 1H), 1.66 (s, 3H), 1.65-1.56 (m, 3H), 1.52 (s, 3H), 1.51-1.41 (m, 1H), 1.28 (s, 9H), 0.80 (s, 9H). ESI-MS m/z calc. 698.3727, found 699.3 (M+1)⁺; Retention time: 2.27 minutes (LC method A).

### Example 151: Preparation of (14S)-8-tert-Butyl-17-{4-[1-(3,3-dimethylbutyl)-1H-pyrazol-3-yl]pyridin-2-yl}-12,12-dimethyl-2λ⁶-thia-3,9,11,18,23-pentaazatetracyclo [17.3.1.111,14.05,10]tetracosa-1(23),5(10),6,8,19,21-hexaene-2,2,4-trione (Compound 414)

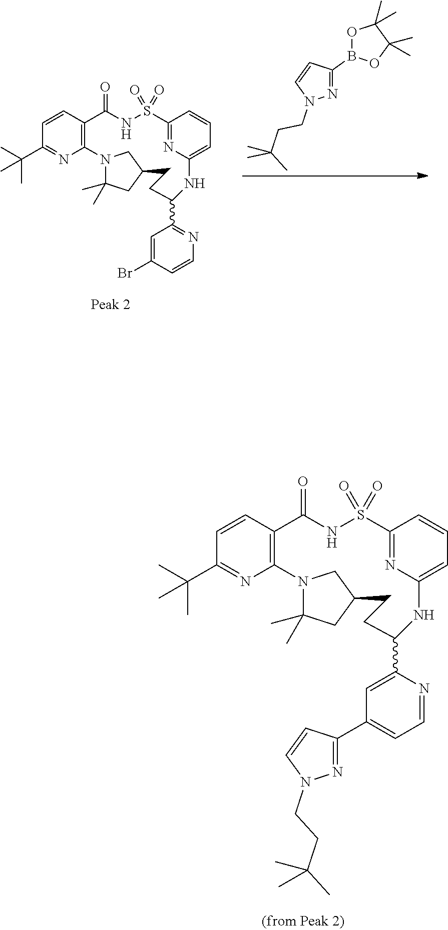 [SMILES: CC(C)(C)c1ccc2c(n1)N1C[C@@H](CCC(c3cc(Br)ccn3)Nc3cccc(n3)S(=O)(=O)NC2=O)CC1(C)C ; CC(C)(C)CCn1ccc(B2OC(C)(C)C(C)(C)O2)n1 ; CC(C)(C)CCn1ccc(-c2ccnc(C3CC[C@@H]4CN(c5nc(C(C)(C)C)ccc5C(=O)NS(=O)(=O)c5cccc(n5)N3)C(C)(C)C4)c2)n1]

In a 1-dram vial, Peak 2 of (14S)-17-(4-bromopyridin-2-yl)-8-tert-butyl-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (21.7 mg, 0.03458 mmol) and 1-(3,3-dimethylbutyl)-3-(4,4,5,5-tetramethyl-1,3,2-dioxaborolan-2-yl)pyrazole (22.5 mg, 0.08088 mmol) were mixed with dioxane (500 μL), to which Pd(dppf)Cl₂-DCM (3.1 mg, 0.003796 mmol) and aqueous Na₂CO₃ (80 4 of 2.0 M, 0.1600 mmol) were added. The resulting mixture was sparged with nitrogen gas, then stirred at 110° C. for 18 h. It was then cooled to room temperature, diluted with MeOH (600 μL), filtered, and purified by reverse-phase preparative chromatography using a C₁₈ column and a gradient eluent of 1 to 99% MeCN in H₂O containing 5 mM HCl solution to give a beige solid, (14S)-8-tert-butyl-17-{4-[1-(3,3-dimethylbutyl)-1H-pyrazol-3-yl]pyridin-2-yl}-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Hydrochloride salt) (10.7 mg, 42%); ¹H NMR (400 MHz, DMSO-d₆) δ 12.47 (s, 1H), 8.72 (d, J=5.3 Hz, 1H), 7.82 (d, J=9.0 Hz, 1H), 7.77 (s, 1H), 7.66 (dd, J=8.5, 7.3 Hz, 1H), 7.64 (d, J=8.0 Hz, 1H), 7.56 (d, J=1.9 Hz, 1H), 7.52 (d, J=5.3 Hz, 1H), 7.13 (d, J=7.2 Hz, 1H), 6.90 (d, J=8.4 Hz, 1H), 6.65 (d, J=8.0 Hz, 1H), 6.58 (d, J=1.9 Hz, 1H), 5.41 (td, J=10.0, 9.3, 4.9 Hz, 1H), 4.14 (t, J=8.2 Hz, 2H), 3.28 (dd, J=10.1, 6.6 Hz, 1H), 2.76 (t, J=10.4 Hz, 1H), 2.32-2.19 (m, 1H), 2.14-2.00 (m, 1H), 1.97-1.87 (m, 1H), 1.85 (dd, J=11.9, 5.4 Hz, 1H), 1.77 (dd, J=14.1, 5.7 Hz, 1H), 1.65 (s, 3H), 1.58 (t, J=12.5 Hz, 1H), 1.51 (s, 3H), 1.50-1.41 (m, 3H), 1.28 (s, 9H), 0.67 (s, 9H). ESI-MS m/z calc. 698.3727, found 699.4 (M+1)⁺; Retention time: 2.17 minutes (LC method A).

### Example 152: Preparation of (5S)-11-tert-Butyl-7,7-dimethyl-3-phenyl-17λ⁶-thia-2,8,10,16,22-pentaazatetracyclo[16.3.1.15,8.09,14]tricosa-1(21),9(14),10,12,18(22),19-hexaene-15,17,17-trione (Compound 415) and (5S)-11-tert-butyl-7,7-dimethyl-3-phenyl-17λ⁶-thia-2,8,10,16,22-pentaazatetracyclo[16.3.1.15,8.09,14]tricosa-1(21),9(14),10,12,18(22),19-hexaene-15,17,17-trione (Compound 416)

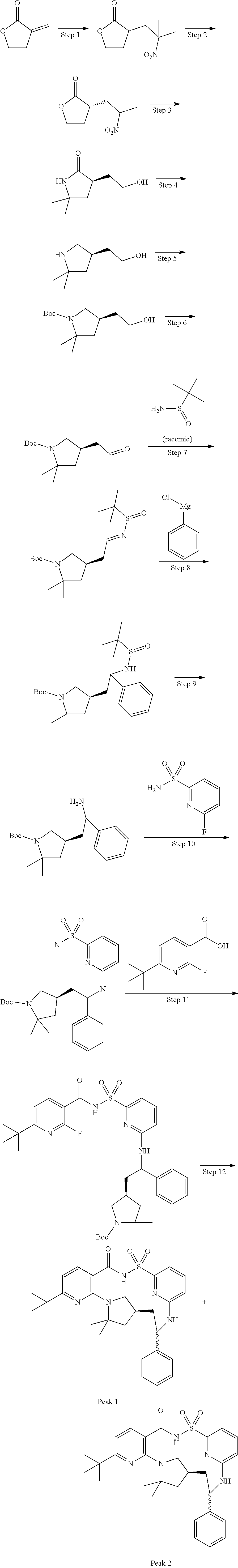

### Step 1: 3-(2-Methyl-2-nitro-propyl)tetrahydrofuran-2-one

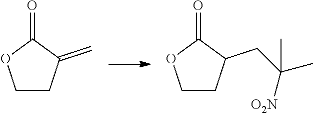

A solution of 3-methylenetetrahydrofuran-2-one (101.11 g, 90.357 mL, 989.46 mmol) dissolved in acetonitrile (1.1 L) was added in a slow stream to a mixture of DBU (23.414 g, 23 mL, 153.80 mmol) and 2-nitropropane (107.14 g, 108 mL, 1.2026 mol). The reaction is exothermic: the addition rate was such that the internal temperature remained below 35° C. during the course of the addition. The resulting solution was stirred for 19 h at room temperature, then was concentrated under vacuum to obtain a light yellow solid. The crude product was stirred overnight in diethyl ether (500 mL), then filtered. The solids were dissolved in DCM (500 mL) and washed with 3.0 M aq. HCl (2×500 mL), H₂O (500 mL), sat. aq. NaHCO₃(500 mL), and sat. aq. NaCl (500 mL). The DCM layer was dried over anhydrous magnesium sulfate, filtered and concentrated under vacuum to give white crystal solids. 3-(2-Methyl-2-nitro-propyl)tetrahydrofuran-2-one (157.59 g, 82%): ¹H NMR (500 MHz, DMSO-d₆) δ 4.27 (td, J=8.6, 8.6, 1.5 Hz, 1H), 4.15-4.00 (m, 1H), 2.68 (dddd, J=11.4, 9.7, 8.5, 2.9 Hz, 1H), 2.43 (dd, J=14.8, 2.9 Hz, 1H), 2.34-2.23 (m, 1H), 2.09-1.99 (m, 1H), 1.85 (qd, J=11.6, 11.6, 11.5, 8.6 Hz, 1H), 1.59 (s, 3H), 1.58 (s, 3H). ESI-MS m/z calc. 187.08446, found 188.2 (M+1)⁺; Retention time: 1.39 minutes (LC Method B).

### Step 2: (3R)-3-(2-Methyl-2-nitro-propyl)tetrahydrofuran-2-one and (3S)-3-(2-methyl-2-nitro-propyl)tetrahydrofuran-2-one

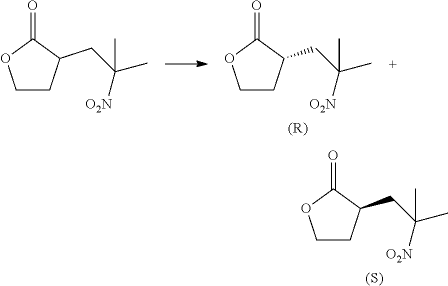

A racemic mixture of 3-(2-methyl-2-nitro-propyl)tetrahydrofuran-2-one (173.55 g, 927.1 mmol) was separated by Chiral SFC, using an AD-H (2×25 cm) column, and 35% methanol in CO₂, 100 bar as the eluent. The flow rate was 70 mL/min (220 nm) and the injection volume was 3 mL, of a 20 mg/mL solution in methanol:DCM. The two isomers were isolated as solids after drying cold with the addition of acetonitrile to prevent concentration in methanol.

Peak 1, (3R)-3-(2-Methyl-2-nitro-propyl)tetrahydrofuran-2-one (88 g, 50%). ¹H NMR (400 MHz, DMSO-d₆) δ 4.27 (td, J=8.6, 1.5 Hz, 1H), 4.09 (ddd, J=10.9, 8.7, 6.1 Hz, 1H), 2.76-2.63 (m, 1H), 2.43 (dd, J=14.8, 2.9 Hz, 1H), 2.35-2.24 (m, 1H), 2.04 (dd, J=14.8, 10.0 Hz, 1H), 1.94-1.79 (m, 1H), 1.59 (s, 3H), 1.58 (s, 3H). ESI-MS m/z calc. 187.08446, Retention time: 0.71 minutes (mass not detected) (LC Method A).

Peak 2, (3S)-3-(2-Methyl-2-nitro-propyl)tetrahydrofuran-2-one (80 g, 46%). ¹H NMR (400 MHz, DMSO-d₆) δ 4.27 (td, J=8.6, 1.5 Hz, 1H), 4.09 (ddd, J=10.9, 8.7, 6.1 Hz, 1H), 2.76-2.62 (m, 1H), 2.43 (dd, J=14.8, 2.9 Hz, 1H), 2.33-2.23 (m, 1H), 2.04 (dd, J=14.8, 10.0 Hz, 1H), 1.91-1.78 (m, 1H), 1.59 (s, 3H), 1.58 (s, 3H). ESI-MS m/z calc. 187.08446, Retention time: 0.76 minutes (mass not detected) (LC Method A).

### Step 3: (3R)-3-(2-Hydroxyethyl)-5,5-dimethyl-pyrrolidin-2-one

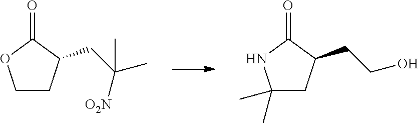

To a solution of (3R)-3-(2-methyl-2-nitro-propyl)tetrahydrofuran-2-one (37 g, 197.66 mmol) in ethanol (370.00 mL) was added Raney Nickel (8 g, 91.230 mmol) in a Parr reactor. The reactor was stirred at 80° C. under hydrogen (3 bar) for 48 h. Celite (10 g) was added and the resulting slurry was filtered. The solids were washed with ethanol (2×150 mL) and discarded. The combined filtrates were concentrated under vacuum to obtain a yellow solid. The solid was then triturated with ether overnight and the resulting solid was collected to yield (3R)-3-(2-hydroxyethyl)-5,5-dimethyl-pyrrolidin-2-one (26.9 g, 82%) as a white solid. ¹H NMR (500 MHz, DMSO-d₆) δ 7.68 (s, 1H), 4.54 (m, 1H), 3.47 (m, 1H), 3.41 (m, 1H), 2.48-2.42 (m, 1H), 2.04 (m, 1H), 1.86 (m, 1H), 1.42 (m, 1H), 1.30 (m, 1H), 1.18 (s, 3H), 1.13 (s, 3H).

### Step 4: 2-[(3R)-5,5-Dimethylpyrrolidin-3-yl]ethanol

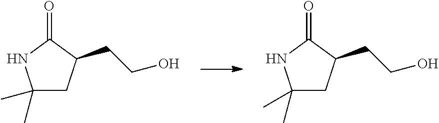

LAH (40 g, 1.0539 mol) was added in small portions to a cooled (0° C.) solution of (3R)-3-(2-hydroxyethyl)-5,5-dimethyl-pyrrolidin-2-one (31.5 g, 200.37 mmol) in THF (350 mL). The resulting slurry was allowed to warm to room temperature and stirred for 1 h. It was then heated to 70° C. for 48 h. After cooling to room temperature, the reaction was cooled to 0° C. using an ice bath. Water (40 mL) was slowly added, followed by 15% w/v aq. NaOH solution (40 mL). Another 120 mL of water was added and the ice bath was removed. The quenched reaction mixture was diluted with diethyl ether (200 mL) and was allowed to warm and stir at room temperature for 30 min. The mixture was filtered through Celite and the solids were washed with diethyl ether (2×100 mL). The combined filtrates were then dried over anhydrous sodium sulfate and filtered. The combined filtrate was evaporated in vacuo to give 2-[(3R)-5,5-dimethylpyrrolidin-3-yl]ethanol (32 g, 100%) as a yellowish oil. ¹H NMR (500 MHz, DMSO-d₆) δ 4.40 (s, 1H), 3.46-3.39 (m, 2H), 2.97-2.90 (m, 1H), 2.44-2.32 (m, 1H), 2.19-2.00 (m, 1H), 1.71-1.62 (m, 1H), 1.49-1.35 (m, 2H), 1.09-0.97 (m, 7H).

### Step 5: tert-Butyl (4R)-4-(2-hydroxyethyl)-2,2-dimethyl-pyrrolidine-1-carboxylate

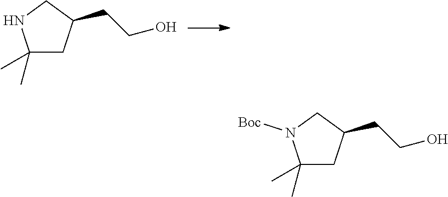

A solution of 2-[(3R)-5,5-dimethylpyrrolidin-3-yl]ethanol (32 g, 203.31 mmol) in DCM (160.00 mL) was cooled to 0° C., then a solution of NaOH (19 g, 475.03 mmol) in H₂O (160 mL) was added. A solution of tert-butoxycarbonyl tert-butyl carbonate (59 g, 62.105 mL, 270.34 mmol) in DCM (160 mL) was added dropwise to the chilled amine mixture, then the ice water bath was removed and the reaction was allowed to stir at room temperature for 22 h. The phases were then separated: the aqueous phase was extracted with DCM (150 mL). The organic phase was dried over sodium sulfate and concentrated in vacuo, then purified by silica gel chromatography using 3% MeOH in DCM to give tert-butyl (4R)-4-(2-hydroxyethyl)-2,2-dimethyl-pyrrolidine-1-carboxylate (30.915 g, 60%) as a clear oil. ¹H NMR (500 MHz, DMSO-d₆) δ 4.42 (t, J=5.1, 1H), 3.56 (q, J=8.7 Hz, 1H), 3.42-3.36 (m, 2H), 2.79 (dt, J=12.8, 10.6 Hz, 1H), 2.21-2.10 (m, 1H), 1.92-1.84 (m, 1H), 1.47-1.44 (m, 2H), 1.37 (m, 13H), 1.24 (s, 3H). ESI-MS m/z calc. 243.18344, found 244.5 (M+1)⁺; Retention time: 2.17 minutes (LC Method H).

### Step 6: tert-Butyl (4R)-2,2-dimethyl-4-(2-oxoethyl)pyrrolidine-1-carboxylate

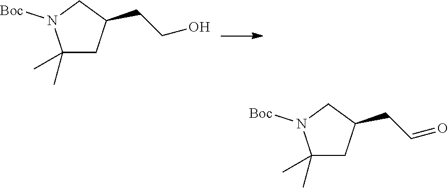 [SMILES: CC(C)(C)OC(=O)N1C[C@@H](CCO)CC1(C)C ; CC(C)(C)OC(=O)N1C[C@@H](CC=O)CC1(C)C]

A buffered solution of bleach was prepared by dissolving sodium bicarbonate (9.53 g, 113.44 mmol) into a solution of aqueous sodium hypochlorite (136 mL of 1.6 M, 217.60 mmol) and stirring at 0° C. in an ice bath. That solution was then added dropwise to a second solution stirred mechanically, that was prepared in advance by adding sodium bromide (944 mg, 9.1745 mmol) (dissolved in water (5 mL) and TEMPO (73 mg, 0.4672 mmol) to tert-butyl (4R)-4-(2-hydroxyethyl)-2,2-dimethyl-pyrrolidine-1-carboxylate (43.68 g, 179.50 mmol) in DCM (570 mL) maintained at −12° C. During the addition of the buffered bleach solution to the substrate solution the internal temperature was maintained below −10° C. After completion of the addition, the reaction mixture was maintained at −14° C. for 10 min. The excess bleach was quenched with ethanol (2.3670 g, 3 mL, 51.380 mmol). The reaction mixture was concentrated under reduced pressure at 35° C. The resulting residue was partitioned between ethyl acetate (200 mL) and water (100 mL). The aqueous phase was separated and extracted with ethyl acetate (100 mL). The organics were combined, washed with brine (150 mL), dried with sodium sulfate, filtered and concentrated under reduced pressure to provide tert-butyl (4R)-2,2-dimethyl-4-(2-oxoethyl)pyrrolidine-1-carboxylate (34.6 g, 69%)¹H NMR (400 MHz, CDCl₃) δ 9.78 (s, 1H), 3.87-3.70 (m, 1H), 3.01-2.82 (m, 1H), 2.68-2.41 (m, 3H), 2.07-1.94 (m, 1H), 1.53-1.30 (m, 16H). ESI-MS m/z calc. 241.1678, found 186.2 (M-tBu+2H=M−55)⁺; Retention time: 1.79 minutes (LC method E).

### Step 7: tert-Butyl (4R)-4-[(2E)-2-tert-butylsulfinyliminoethyl]-2,2-dimethyl-pyrrolidine-1-carboxylate

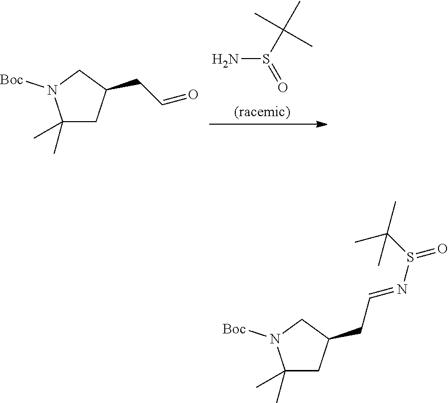

To a solution of tert-butyl (4R)-2,2-dimethyl-4-(2-oxoethyl)pyrrolidine-1-carboxylate (15.74 g, 61.962 mmol) in DCM (125 mL) were added magnesium sulfate (48 g, 398.78 mmol), racemic 2-methylpropane-2-sulfinamide (13.2 g, 108.91 mmol) and pyridinium p-toluenesulfonate (904 mg, 3.5829 mmol). The reaction mixture was stirred at room temperature for 24 h. The mixture was filtered and purified by silica gel chromatography eluting with 0% to 50% ethyl acetate in heptane to give tert-butyl (4R)-4-[(2E)-2-tert-butylsulfinyliminoethyl]-2,2-dimethyl-pyrrolidine-1-carboxylate (20.1 g, 89%) as a white solid. ¹H NMR (400 MHz, CDCl₃) δ 8.11-8.00 (m, 1H), 3.88-3.60 (m, 1H), 3.05-2.86 (m, 1H), 2.68-2.45 (m, 3H), 2.07-1.91 (m, 1H), 1.64-1.13 (m, 25H). ESI-MS m/z calc. 344.2134, found 245.2 (M-Boc+2H=M−99)⁺; Retention time: 1.95 minutes (LC method E).

### Step 8: tert-Butyl (4S)-4-[2-(tert-butylsulfinylamino)-2-phenyl-ethyl]-2,2-dimethyl-pyrrolidine-1-carboxylate

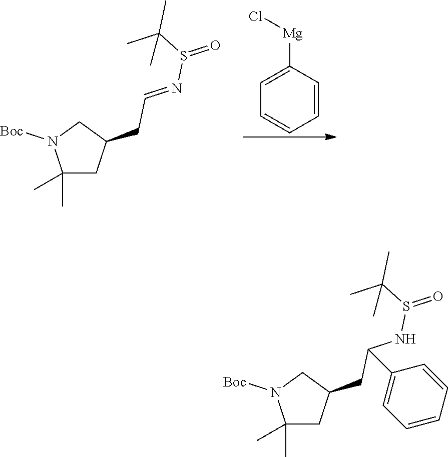

A solution of chloro(phenyl)magnesium in THF (3.0 mL of 2 M, 6.0 mmol) was added dropwise to a solution of tert-butyl (4R)-4-[(2E)-2-tert-butylsulfinyliminoethyl]-2,2-dimethyl-pyrrolidine-1-carboxylate (1.4 g, 3.9418 mmol) in THF (14 mL) at 78° C. and the mixture was stirred at 78° C. for 30 min. The reaction was warmed to room temperature over 5 h. An aqueous saturated solution of NH₄Cl (50 mL) was added. The product was extracted with EtOAc (3×50 mL). The combined organic phases were washed with brine (50 mL), dried over Na₂SO₄, filtered, and evaporated to dryness (1.79 g of crude with a purity of 84.6%). This mixture was purified by silica gel chromatography (eluting with 0 to 50% ethyl acetate in hexanes) to give tert-butyl (4S)-4-[2-(tert-butylsulfinylamino)-2-phenyl-ethyl]-2,2-dimethyl-pyrrolidine-1-carboxylate (1.51 g, 69%) as a colorless oil. ¹H NMR (400 MHz, CDCl₃) δ 7.40-7.28 (m, 5H), 4.39-4.25 (m, 1H), 3.77-3.23 (m, 2H), 3.02-2.66 (m, 1H), 2.11-1.69 (m, 4H), 1.49-1.12 (m, 24H). ESI-MS m/z calc. 422.2603, found 323.2 (M-Boc+2H=M−99)⁺; Retention time: 1.98 minutes (LC method E).

### Step 9: tert-Butyl (4S)-4-(2-amino-2-phenyl-ethyl)-2,2-dimethyl-pyrrolidine-1-carboxylate

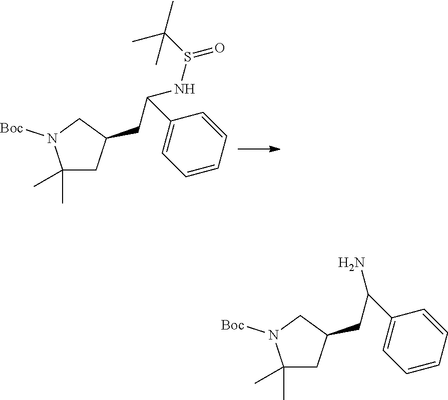 [SMILES: CC(C)(C)OC(=O)N1C[C@@H](CC(NS(=O)C(C)(C)C)c2ccccc2)CC1(C)C ; CC(C)(C)OC(=O)N1C[C@@H](CC(N)c2ccccc2)CC1(C)C]

Iodine (66 mg, 0.2600 mmol) was added to a solution of tert-butyl (4S)-4-[2-(tert-butylsulfinyl amino)-2-phenyl-ethyl]-2,2-dimethyl-pyrrolidine-1-carboxylate (1.49 g, 3.5256 mmol) in THF (11 mL) and water (2.3 mL). The reaction was stirred at 60° C. for 2 h. Iodine (78 mg, 0.3073 mmol) was added and the reaction was stirred at 60° C. for 2.5 h. Iodine (71 mg, 0.2797 mmol) was added, the reaction was stirred at 60° C. for 1.5 h. After cooling to room temperature, the reaction was diluted with ethyl acetate (25 mL) and washed successively with aqueous 5% sodium bicarbonate solution (2×25 mL), aqueous 10% sodium thiosulfate solution (2×25 mL), and brine (50 mL). The organic phase was dried over sodium sulfate, filtered and evaporated to dryness to give tert-butyl (4S)-4-(2-amino-2-phenyl-ethyl)-2,2-dimethyl-pyrrolidine-1-carboxylate (1.27 g, 92%) as a pale yellow oil. ¹H NMR (400 MHz, CDCl₃) δ 7.39-7.23 (m, 5H), 3.92-3.84 (m, 1H), 3.80-3.39 (m, 1H), 3.03-2.73 (m, 1H), 2.17-1.85 (m, 4H), 1.84-1.66 (m, 3H), 1.49-1.33 (m, 12H), 1.29-1.17 (m, 3H). ESI-MS m/z calc. 318.23074, found 319.2 (M+1)⁺; Retention time: 1.39 minutes (LC method E).

### Step 10: tert-Butyl (4S)-2,2-dimethyl-4-[2-phenyl-2-[(6-sulfamoyl-2-pyridyl)amino]ethyl]pyrrolidine-1-carboxylate

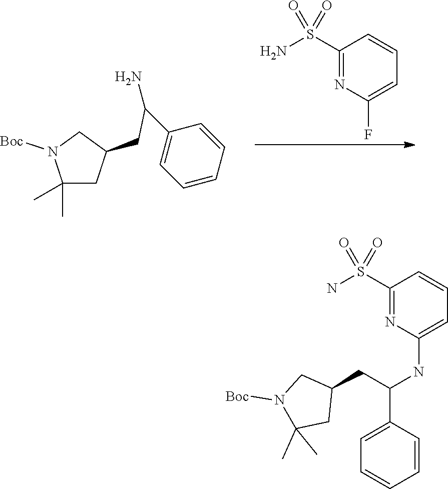 [SMILES: CC(C)(C)OC(=O)N1C[C@@H](CC(N)c2ccccc2)CC1(C)C ; NS(=O)(=O)c1cccc(F)n1 ; CC(C)(C)OC(=O)N1C[C@@H](CC(Nc2cccc(S(N)(=O)=O)n2)c2ccccc2)CC1(C)C]

To a solution of tert-butyl (4S)-4-(2-amino-2-phenyl-ethyl)-2,2-dimethyl-pyrrolidine-1-carboxylate (1.25 g, 3.2069 mmol) and 6-fluoropyridine-2-sulfonamide (1.69 g, 9.5931 mmol) in acetonitrile (11 mL) was added DIPEA (1.4840 g, 2 mL, 11.482 mmol). The reaction tube was sealed and the mixture was stirred at 120° C. for 45 h. The reaction was partitioned between ethyl acetate (50 mL) and water (100 mL). The aqueous phase was extracted with ethyl acetate (50 mL). The combined organic layers were washed with brine (100 mL), dried over sodium sulfate, filtered and concentrated. The crude material was purified by reverse phase chromatography on C₁₈ (eluting with 5 to 100% methanol in water+0.1% HCOOH). The product fractions were combined and the organic solvents were removed under reduced pressure. Aqueous 5% NaHCO₃ solution (20 mL) was added to the aqueous mixture, which was then extracted with ethyl acetate (2×20 mL). The combined organic layers were dried over anhydrous sodium sulfate, filtered and concentrated under reduced pressure to give tert-butyl (4S)-2,2-dimethyl-4-[2-phenyl-2-[(6-sulfamoyl-2-pyridyl)amino]ethyl]pyrrolidine-1-carboxylate (583 mg, 38%) as a beige solid. ¹H NMR (400 MHz, DMSO-d₆) δ 7.55-7.38 (m, 4H), 7.34-7.24 (m, 2H), 7.23-7.16 (m, 1H), 7.09-7.02 (m, 2H), 6.95 (d, J=7.1 Hz, 1H), 6.63 (dd, J=8.4, 2.6 Hz, 1H), 5.22-4.91 (m, 1H), 3.60-3.42 (m, 1H), 2.93-2.69 (m, 1H), 2.22-1.98 (m, 1H), 1.97-1.82 (m, 2H), 1.82-1.65 (m, 1H), 1.63-1.42 (m, 1H), 1.41-1.24 (m, 12H), 1.19-1.06 (m, 3H). ESI-MS m/z calc. 474.23007, found 419.2 (M-tBu+2H=M−55)⁺; Retention time: 4.3 minutes (LC method F).

### Step 11: tert-Butyl (4S)-4-[2-[[6-[(6-tert-butyl-2-fluoro-pyridine-3-carbonyl)sulfamoyl]-2-pyridyl]amino]-2-phenyl-ethyl]-2,2-dimethyl-pyrrolidine-1-carboxylate

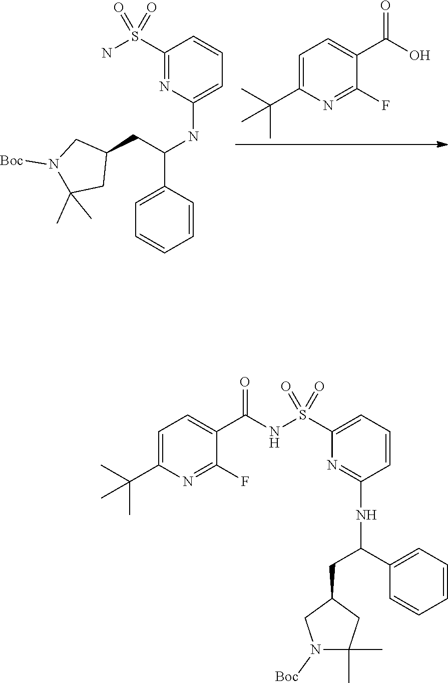

A mixture of 6-tert-butyl-2-fluoro-pyridine-3-carboxylic acid (85.9 mg, 0.4356 mmol) and CDI (76.3 mg, 0.4706 mmol) were dissolved in THF (600 μL), and the mixture was stirred at room temperature for 16 h. tert-Butyl (4S)-2,2-dimethyl-4-[2-phenyl-2-[(6-sulfamoyl-2-pyridyl)amino]ethyl]pyrrolidine-1-carboxylate (109.8 mg, 0.2313 mmol) was added, followed by DBU (140 4, 0.9362 mmol), and the resulting mixture was stirred for an additional 24 h. The mixture was diluted with ethyl acetate and washed with 1 N HCl, brine, dried over sodium sulfate, and concentrated. The residue was purified by silica gel chromatography eluting with 0-100% ethyl acetate in hexanes to give tert-butyl (4S)-4-[2-[[[6(6-tert-butyl-2-fluoro-pyridine-3-carbonyl)sulfamoyl]-2-pyridyl]amino]-2-phenyl-ethyl]-2,2-dimethyl-pyrrolidine-1-carboxylate (120 mg, 79%) as a clear oil. ESI-MS m/z calc. 653.30475, found 654.4 (M+1)⁺; Retention time: 0.87 minutes (LC method D).

### Step 12: (5S)-11-tert-Butyl-7,7-dimethyl-3-phenyl-17λ⁶-thia-2,8,10,16,22-pentaazatetracyclo[16.3.1.15,8.09,14]tricosa-1(21),9(14),10,12,18(22),19-hexaene-15,17,17-trione (Compound 415) and (5S)-11-tert-butyl-7,7-dimethyl-3-phenyl-17λ⁶-thia-2,8,10,16,22-pentaazatetracyclo[16.3.1.15,8.09,14]tricosa-1(21),9(14),10,12,18(22),19-hexaene-15,17,17-trione (Compound 416)

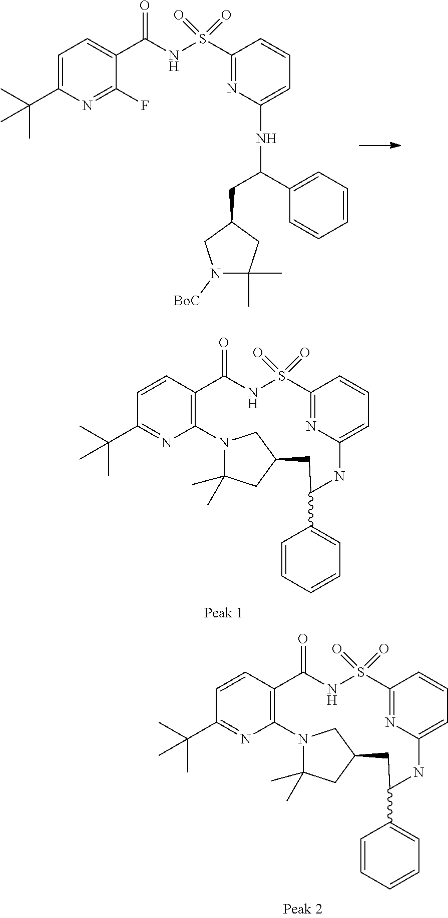

Stage 1: tert-Butyl (4S)-4-[2-[[6-[(6-tert-butyl-2-fluoro-pyridine-3-carbonyl)sulfamoyl]-2-pyridyl]amino]-2-phenyl-ethyl]-2,2-dimethyl-pyrrolidine-1-carboxylate (120 mg, 0.1835 mmol) and TFA (150 μL, 1.947 mmol) were combined in DCM (2 mL) and stirred at room temperature for 2 h. The reaction mixture was evaporated to give 6-tert-butyl-N-[[6-[[2-[(3S)-5,5-dimethylpyrrolidin-3-yl]-1-phenyl-ethyl]amino]-2-pyridyl]sulfonyl]-2-fluoro-pyridine-3-carboxamide (trifluoroacetate salt).

Stage 2: The product from above was dissolved in NMP (6 mL) with potassium carbonate (331 mg, 2.395 mmol) and heated at 120° C. for 16 h. The mixture was cooled to room temperature and partitioned between ethyl acetate and 1 N HCl. The organics were separated, washed with brine, dried over sodium sulfate and evaporated. The crude material was purified by silica gel chromatography eluting with 0-100% ethyl acetate in hexanes to give (5S)-11-tert-butyl-7,7-dimethyl-3-phenyl-17λ⁶-thia-2,8,10,16,22-pentaazatetracyclo[16.3.1.15,8.09,14]tricosa-1(21),9(14),10,12,18(22),19-hexaene-15,17,17-trione as a mixture of diastereomers (26.4 mg). The diastereomers were separated by SFC chromatography: ChiralPak IG (250×10 mm), 5 μm column at 35° C., eluant: 22% MeOH (20 mM NH₃), 78% CO₂, flow rate: 10 mL/min, concentration: 20 mg/mL in methanol (no modifier), injection volume: 70 μL, pressure: 154 bar, wavelength: 210 nm. This gave:

Peak 1: (5S)-11-tert-butyl-7,7-dimethyl-3-phenyl-17λ⁶-thia-2,8,10,16,22-pentaazatetracyclo[16.3.1.15,8.09,14]tricosa-1 (21),9(14),10,12,18(22),19-hexaene-15,17,17-trione (10.0 mg, 10%); ESI-MS m/z calc. 533.24603, found 534.5 (M+1)⁺; Retention time: 2.16 minutes (LC method A).

Peak 2: (5S)-11-tert-butyl-7,7-dimethyl-3-phenyl-17λ⁶-thia-2,8,10,16,22-pentaazatetracyclo[16.3.1.15,8.09,14]tricosa-1(21),9(14),10,12,18(22),19-hexaene-15,17,17-trione (2.4 mg, 2%); ESI-MS m/z calc. 533.24603, found 534.5 (M+1)⁺; Retention time: 2.17 minutes (LC method A).

### Example 153: Preparation of (14S)-8-tert-Butyl-17-(4-tert-butylpyridin-2-yl)-12,12-dimethyl-2λ⁶-thia-3,11,18,23-tetraazatetracyclo[17.3.1.111,14.05,10]tetracosa-1(23),5,7,9,19,21-hexaene-2,2,4-trione (Compound 417) and (14.9-7-Bromo-8-tert-butyl-17-(4-tert-butylpyridin-2-yl)-12,12-dimethyl-2λ⁶-thia-3,11,18,23-tetraazatetracyclo[17.3.1.111,14.05,10]tetracosa-1(23),5,7,9,19,21-hexaene-2,2,4-trione (Compound 418)

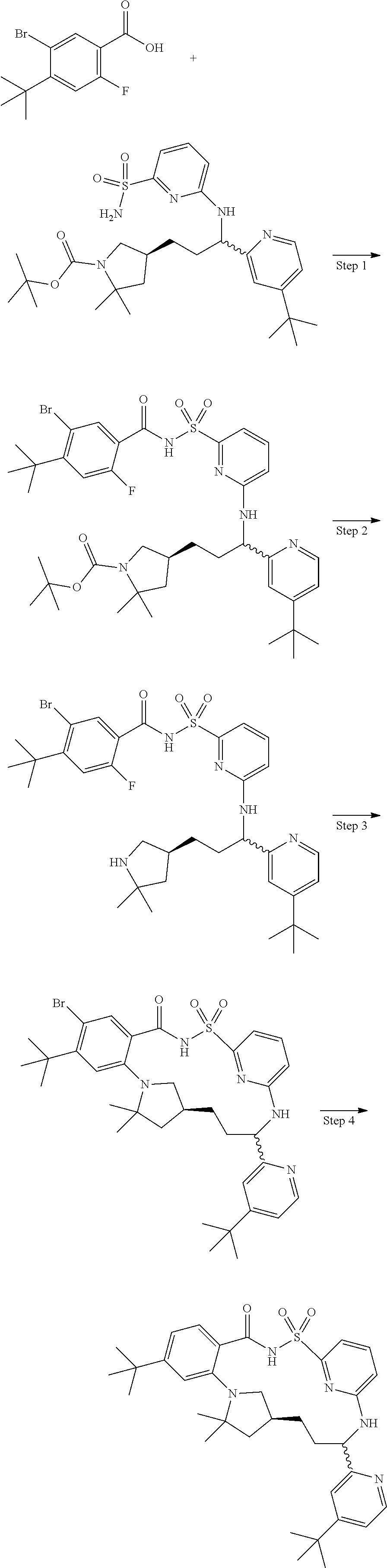

### Step 1: tert-Butyl (4S)-4-[3-[[6-[(5-bromo-4-tert-butyl-2-fluoro-benzoyl)sulfamoyl]-2-pyridyl]amino]-3-(4-tert-butyl-2-pyridyl)propyl]-2,2-dimethyl-pyrrolidine-1-carboxylate

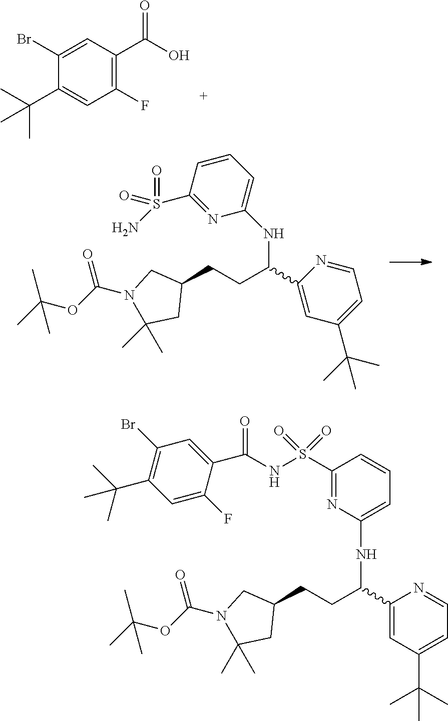

A round-bottomed flask was charged with 4-tert-butyl-2-fluoro-benzoic acid (230 mg, 1.1370 mmol) and THF (7 mL). 1,1′-Carbonyldiimidazole (185 mg, 1.1409 mmol) was added and the mixture was stirred under nitrogen at room temperature for 2 h. In a separate flask, a solution of tert-butyl (4S)-4-[3-(4-tert-butyl-2-pyridyl)-3-[(6-sulfamoyl-2-pyridyl)amino]propyl]-2,2-dimethyl-pyrrolidine-1-carboxylate (520 mg, 0.9528 mmol) in THF (7 mL) was prepared under nitrogen and it was subsequently added via syringe into the activated acid solution. 1,8-Diazabicyclo[5.4.0]undec-7-ene (610.80 mg, 0.6 mL, 4.0122 mmol) was added and the reaction mixture was stirred at room temperature under nitrogen for 18 h. The solvents were then removed under reduced pressure. The crude material was directly purified by reverse phase chromatography eluting with a gradient of 5% to 100% MeCN in water (with 0.1% of formic acid as a modifier) to afford tert-butyl (4S)-4-[3-[[6-[(5-bromo-4-tert-butyl-2-fluoro-benzoyl)sulfamoyl]-2-pyridyl]amino]-3-(4-tert-butyl-2-pyridyl)propyl]-2,2-dimethyl-pyrrolidine-1-carboxylate (300 mg, 36%) as a yellow solid. ¹H NMR (400 MHz, CDCl₃) δ 8.49 (d, J=5.4 Hz, 1H), 7.97 (d, J=7.3 Hz, 1H), 7.58-7.52 (m, 2H), 7.22-7.15 (m, 2H), 6.62-6.57 (m, 1H), 5.89 (d, J=7.1 Hz, 1H), 5.18-5.08 (m, 1H), 4.11-4.00 (m, 1H), 2.77 (t, J=10.8 Hz, 1H), 2.32-2.19 (m, 1H), 2.14-2.03 (m, 1H), 1.98-1.83 (m, 1H), 1.80-1.70 (m, 2H), 1.49 (s, 10H), 1.44 (s, 8H), 1.34-1.23 (m, 19H). ¹⁹F NMR (377 MHz, CDCl₃) δ −113.56 (br. s., 1F). ESI-MS m/z calc. 801.29346, found 802.2 (M+1)⁺; Retention time: 2.19 minutes (LC method E).

### Step 2: 5-Bromo-4-tert-butyl-N-[[6-[[1-(4-tert-butyl-2-pyridyl)-3-[(3S)-5,5-dimethylpyrrolidin-3-yl]propyl]amino]-2-pyridyl]sulfonyl]-2-fluoro-benzamide

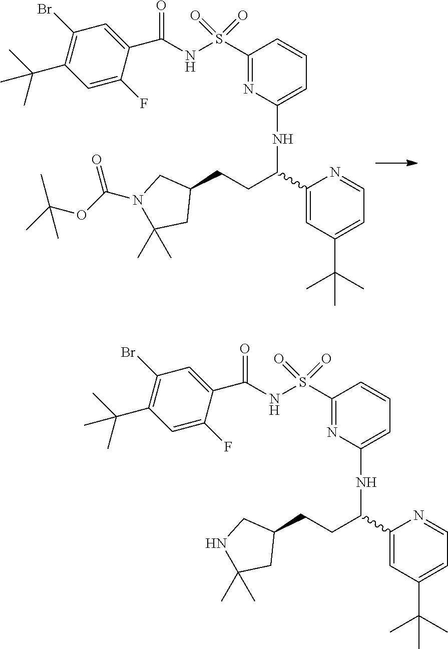

In a round-bottomed flask, to a solution of tert-butyl (4S)-4-[3-[[6-[(5-bromo-4-tert-butyl-2-fluoro-benzoyl)sulfamoyl]-2-pyridyl]amino]-3-(4-tert-butyl-2-pyridyl)propyl]-2,2-dimethyl-pyrrolidine-1-carboxylate (300 mg, 0.3385 mmol) in DCM (4 mL) was added 4 M HCl in dioxane (2 mL of 4 M, 8.0000 mmol), which was then stirred at room temperature for 2 h. Solvent was evaporated to afford crude 5-bromo-4-tert-butyl-N-[[6-[[1-(4-tert-butyl-2-pyridyl)-3-[(3S)-5,5-dimethylpyrrolidin-3-yl]propyl]amino]-2-pyridyl]sulfonyl]-2-fluoro-benzamide (hydrochloride salt) (297 mg, 107%) as a light yellow solid. ¹H NMR (400 MHz, DMSO-d₆) δ 9.23-8.94 (m, 2H), 8.51 (d, J=6.1 Hz, 1H), 8.15 (d, J=8.1 Hz, 1H), 7.85 (br. s., 1H), 7.76-7.60 (m, 3H), 7.33 (d, J=13.0 Hz, 1H), 7.23 (d, J=7.3 Hz, 1H), 7.00 (d, J=8.3 Hz, 1H), 5.24-5.20 (m, 1H), 3.42-3.28 (m, 1H), 2.91-2.79 (m, 1H), 2.44-2.36 (m, 1H), 2.03-1.91 (m, 3H), 1.60-1.35 (m, 15H), 1.27-1.19 (m, 12H). ¹⁹F NMR (377 MHz, DMSO-d₆) δ −112.94 (br. s., 1F). ESI-MS m/z calc. 701.241, found 702.2 (M+1)⁺; Retention time: 1.51 minutes (LC method E).

### Step 3: (14S)-7-Bromo-8-tert-butyl-17-(4-tert-butylpyridin-2-yl)-12,12-dimethyl-R⁶-thia-3,11,18,23-tetraazatetracyclo [17.3.1.111,14.05,10]tetracosa-1(23),5,7,9,19,21-hexaene-2,2,4-trione (Compound 418)

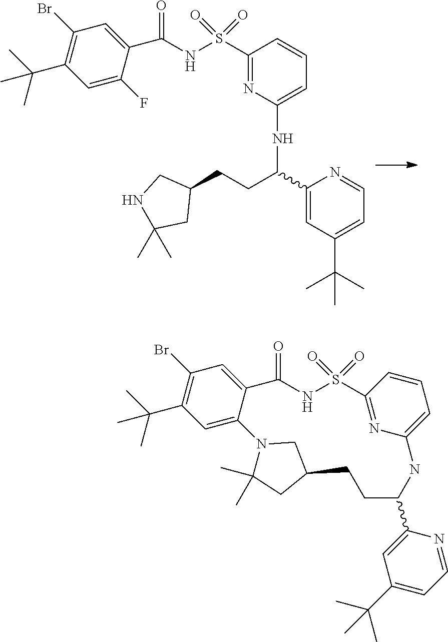

In a flamed-dried reaction tube, 5-bromo-4-tert-butyl-N-[[6-[[1-(4-tert-butyl-2-pyridyl)-3-[(3S)-5,5-dimethylpyrrolidin-3-yl]propyl]amino]-2-pyridyl]sulfonyl]-2-fluoro-benzamide (hydrochloride salt) (240 mg, 0.2917 mmol) was dissolved in DMSO (5 mL), then cesium carbonate (815 mg, 2.5014 mmol) was added. The tube was sealed and heated to 150° C. The reaction mixture was stirred at this temperature for 4 days. The reaction mixture was filtered then purified by reverse phase chromatography using a C₁₈ column and a gradient of 5 to 100% acetonitrile in water (containing 0.1% formic acid) to afford (14S)-7-bromo-8-tert-butyl-17-(4-tert-butylpyridin-2-yl)-12,12-dimethyl-2λ⁶-thia-3,11,18,23-tetraazatetracyclo[17.3.1.111,14.05,10]tetracosa-1(23),5,7,9,19,21-hexaene-2,2,4-trione (35 mg, 17%) as a white solid. ¹H NMR (400 MHz, DMSO-d₆) δ 8.40 (d, J=5.1 Hz, 1H), 7.63-7.57 (m, 1H), 7.47-7.44 (m, 2H), 7.23 (dd, J=5.3, 1.8 Hz, 1H), 7.11-7.00 (m, 2H), 6.85-6.82 (m, 1H), 5.28-5.22 (m, 1H), 3.21 (br. s., 1H), 2.86 (br. s., 1H), 2.22 (br. s., 1H), 1.97-1.83 (m, 3H), 1.72-1.67 (m, 1H), 1.61-1.34 (m, 18H), 1.23 (s, 9H). ESI-MS m/z calc. 681.2348, found 682.3 (M+1)⁺; Retention time: 4.59 minutes (LC method F).

### Step 4: (14S)-8-tert-Butyl-17-(4-tert-butylpyridin-2-yl)-12,12-dimethyl-2λ⁶-thia-3,11,18,23-tetraazatetracyclo[17.3.1.111,14.05,10]tetracosa-1(23),5,7,9,19,21-hexaene-2,2,4-trione (Compound 417)

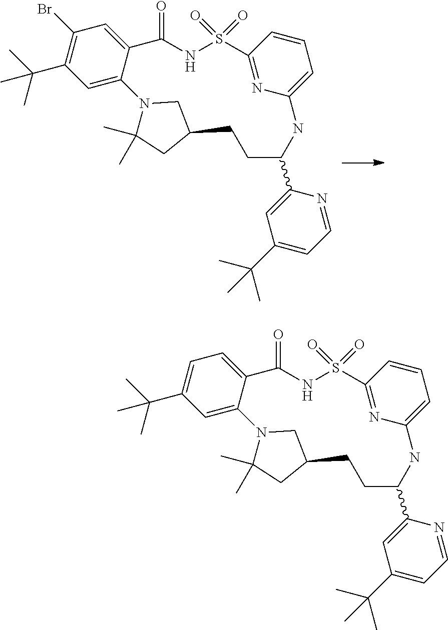

Palladium on carbon (10%, 50% wet, 5% w/w, 30 mg, 0.0141 mmol) was added to a mixture of (14S)-7-bromo-8-tert-butyl-17-(4-tert-butylpyridin-2-yl)-12,12-dimethyl-2λ⁶-thia-3,11,18,23-tetraazatetracyclo[17.3.1.111,14.05,10]tetracosa-1(23),5,7,9,19,21-hexaene-2,2,4-trione (30 mg, 0.0435 mmol) in methanol (1 mL). Hydrogen was bubbled into the suspension for 2 min, and then the reaction mixture was stirred under hydrogen atmosphere for 48 h. The reaction mixture was filtered on Celite and rinsed with methanol. The filtrate was concentrated. The crude material was directly purified by reverse phase chromatography using a C₁₈ column and a gradient of 5% to 100% acetonitrile in water to afford (14S)-8-tert-butyl-17-(4-tert-butylpyridin-2-yl)-12,12-dimethyl-2λ⁶-thia-3,11,18,23-tetraazatetracyclo[17.3.1.111,14.05,10]tetracosa-1(23),5,7,9,19,21-hexaene-2,2,4-trione (15 mg, 57%) as a white solid. ¹H NMR (400 MHz, DMSO-d₆) δ 12.55 (br. s., 1H), 8.41 (d, J=5.4 Hz, 1H), 7.66-7.55 (m, 2H), 7.47 (br. s., 1H), 7.32 (d, J=8.3 Hz, 1H), 7.26-7.23 (m, 1H), 7.09 (d, J=7.1 Hz, 1H), 7.01 (d, J=1.7 Hz, 1H), 6.86 (d, J=8.3 Hz, 1H), 6.75 (dd, J=8.3, 1.5 Hz, 1H), 5.30-5.27 (m, 1H), 3.26-3.19 (m, 1H), 2.91 (t, J=10.1 Hz, 1H), 2.30-2.25 (m, 1H), 1.93-1.89 (m, 3H), 1.72-1.67 (m, 1H), 1.60-1.53 (m, 4H), 1.43-1.39 (m, 1H), 1.36 (s, 3H), 1.27 (s, 9H), 1.24 (s, 9H). ESI-MS m/z calc. 603.32434, found 604.4 (M+1)⁺; Retention time: 3.79 minutes (LC method F).

### Example 154: Preparation of (14S)-8-tert-Butyl-17-[4-(3,3-dimethylbutyl)pyridin-2-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 419)

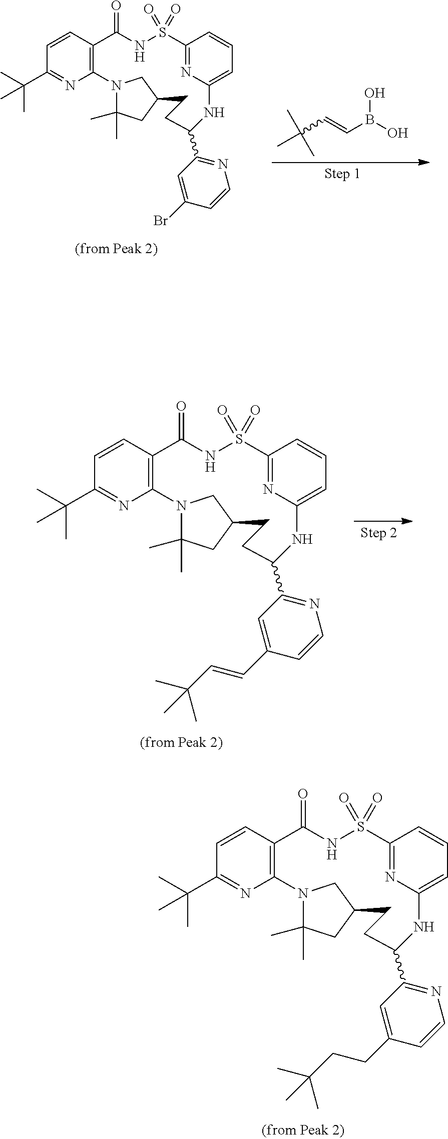

### Step 1: (14S)-8-tert-Butyl-17-{4-[(1E)-3,3-dimethylbut-1-en-1-yl]pyridin-2-yl}-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione

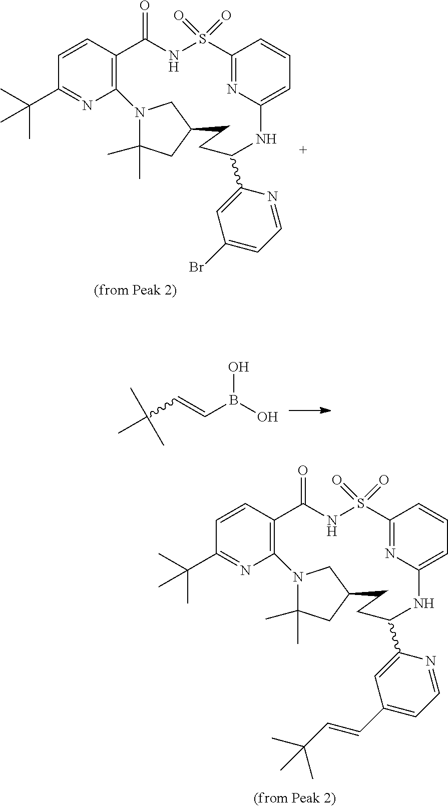

(14S)-17-(4-Bromopyridin-2-yl)-8-tert-butyl-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (51.4 mg, 0.08190 mmol), [(E)-3,3-dimethylbut-1-enyl]boronic acid (22.9 mg, 0.1789 mmol), dichloro[1,1′-bis(diphenylphosphino)ferrocene]palladium (II) dichloromethane adduct (6.9 mg, 0.008449 mmol), and 2 M aq potassium carbonate (125 [IL of 2 M, 0.2500 mmol) were combined in DMF (1 mL) and heated at 90° C. for 16 h. The reaction was filtered and purified by reverse-phase HPLC utilizing a gradient of 1-99% acetonitrile in 5 mM aqueous HCl to yield (14S)-8-tert-butyl-17-14-[(1E)-3,3-dimethylbut-1-en-1-yl]pyridin-2-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (30.9 mg, 60%) as an off-white solid. ESI-MS m/z calc. 630.3352, found 631.4 (M+1)⁺; Retention time: 0.74 minutes (LC method D).

### Step 2: (14S)-8-tert-Butyl-17-[4-(3,3-dimethylbutyl)pyridin-2-yl]-12,12-dimethyl-R⁶-thia-3,9,11,18,23-pentaazatetracyclo[17.3.1.111,14.05,10]tetracosa-1(23),5(10),6,8,19,21-hexaene-2,2,4-trione (Compound 419)

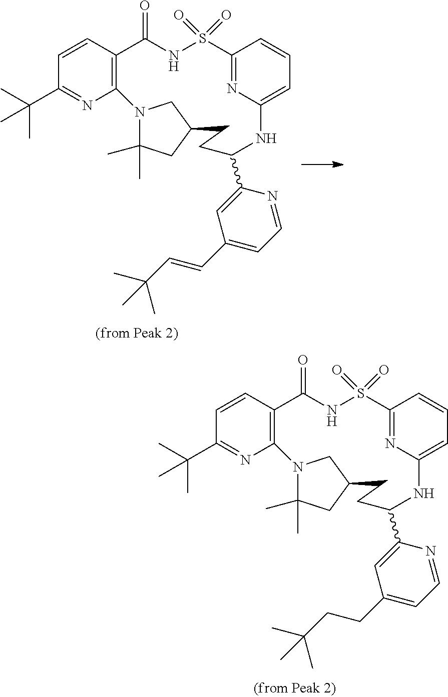 [SMILES: CC(C)(C)/C=C/c1ccnc(C2CC[C@@H]3CN(c4nc(C(C)(C)C)ccc4C(=O)NS(=O)(=O)c4cccc(n4)N2)C(C)(C)C3)c1 ; CC(C)(C)CCc1ccnc(C2CC[C@@H]3CN(c4nc(C(C)(C)C)ccc4C(=O)NS(=O)(=O)c4cccc(n4)N2)C(C)(C)C3)c1]

(14S)-8-tert-Butyl-17-{4-[(1E)-3,3-dimethylbut-1-en-1-yl]pyridin-2-yl}-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (30 mg, 0.04756 mmol) and Pd/C (12 mg of 10% w/w, 0.01128 mmol) were combined in methanol (4 mL) under a balloon of hydrogen. The reaction mixture was stirred at room temperature for 4 h. The reaction was filtered and the solids were washed with methanol. The organics were evaporated and the crude material was purified by silica gel chromatography eluting with 0-100% ethyl acetate in hexanes to give (14S)-8-tert-butyl-17-[4-(3,3-dimethylbutyl)pyridin-2-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione as a white solid (5 mg, 17%). ESI-MS m/z calc. 632.3508, found 633.5 (M+1)⁺; Retention time: 1.94 minutes (LC method A).

### Example 155: Preparation of (14S)-8-tert-Butyl-17-{[5-(3,3-dimethylbutyl)pyridin-2-yl]methyl}-12,12-dimethyl-2λ⁶-thia-3,9,11,18,23-pentaazatetracyclo [17.3.1.111,14.05,10]tetracosa-1(23),5(10),6,8,19,21-hexaene-2,2,4-trione (Compound 420) and (14S)-8-tert-Butyl-17-{[5-(3,3-dimethylbutyl)pyridin-2-yl]methyl}-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 421)

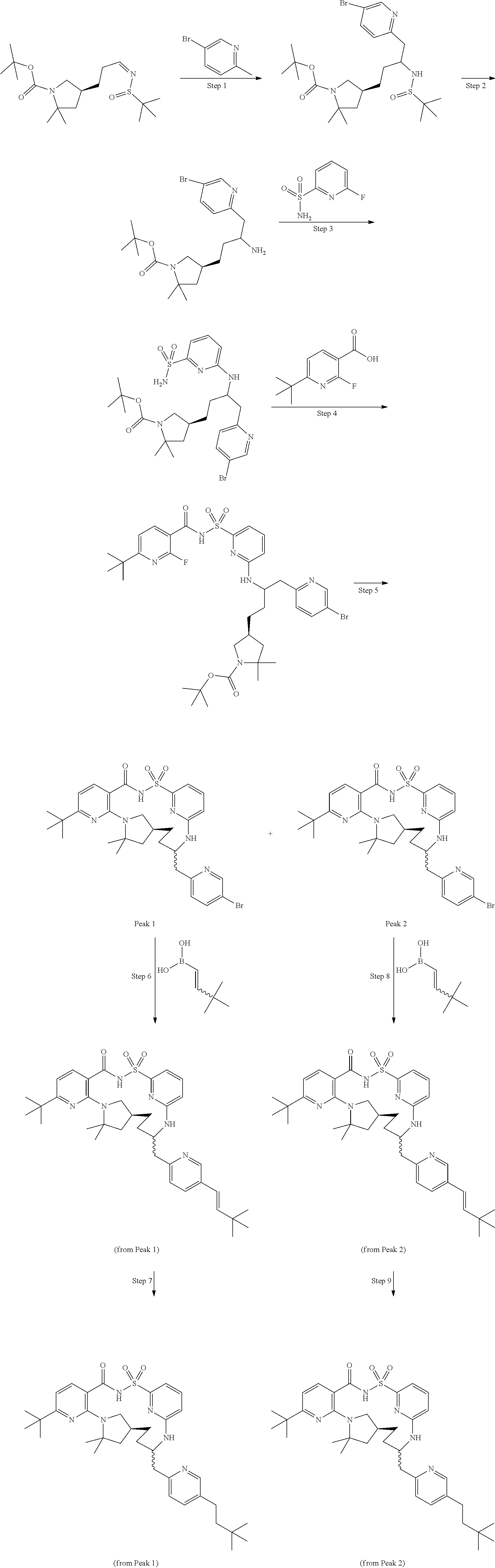

### Step 1: tert-Butyl (4S)-4-[4-(5-bromo-2-pyridyl)-3-(tert-butylsulfinylamino)butyl]-2,2-dimethyl-pyrrolidine-1-carboxylate

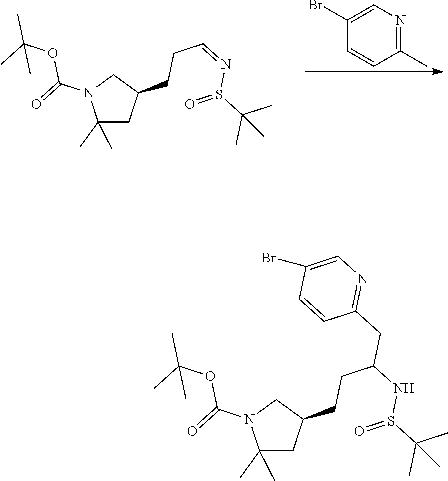

A solution of 5-bromo-2-methyl-pyridine (2.03 g, 11.801 mmol) in THF (10 mL) was slowly added to a solution of LDA (2M in THF/heptane/ethylbenzene) (6 mL of 2 M, 12.000 mmol) in THF (50 mL) at 78° C. The resulting mixture was stirred for 3 h at 78° C. Then, a solution of tert-butyl (4S)-4[(3Z)-3-tert-butylsulfinyliminopropyl]-2,2-dimethyl-pyrrolidine-1-carboxylate (3 g, 7.8653 mmol) in THF (15 mL) was added dropwise at 78° C. and the resulting mixture was stirred at this temperature for 2 h. After slowly allowing to warm to room temperature over 1 h, the resulting mixture was treated with saturated aqueous NH₄Cl solution (80 mL). The product was extracted with ethyl acetate (3×80 mL). The combined extracts were washed with water (80 mL), dried with anhydrous sodium sulfate, filtered and concentrated under reduced pressure. The crude was directly purified by reverse phase chromatography using a gradient of 5 to 100% acetonitrile in water. From the resulting fractions, acetonitrile was evaporated, and the product was extracted with EtOAc (3×80 mL). The combined extracts were washed with brine (80 mL), dried with anhydrous sodium sulfate, filtered and concentrated under reduced pressure. A second purification was performed on silica gel chromatography using a gradient of 70-100% EtOAc in heptanes to give tert-butyl (4S)-4-[4-(5-bromo-2-pyridyl)-3-(tert-butylsulfinylamino)butyl]-2,2-dimethyl-pyrrolidine-1-carboxylate (2.22 g, 53%); ESI-MS m/z calc. 529.1974, found 530.2 (M+1)⁺; Retention time: 2.02 minutes (LC method E).

### Step 2: tert-Butyl (4S)-4-[3-amino-4-(5-bromo-2-pyridyl)butyl]-2,2-dimethyl-pyrrolidine-1-carboxylate

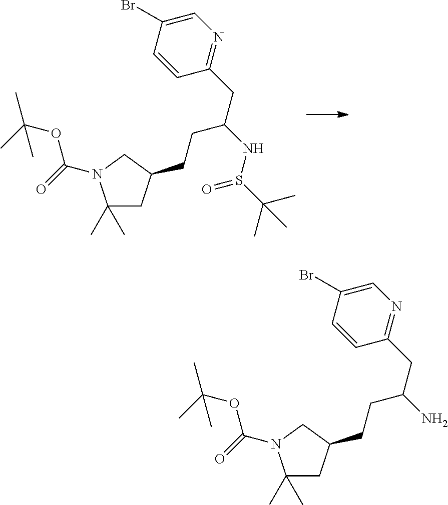

Iodine (600 mg, 0.1217 mL, 2.3640 mmol) was added to a solution of tert-butyl (4S)-4-[4-(5-bromo-2-pyridyl)-3-(tert-butylsulfinylamino)butyl]-2,2-dimethyl-pyrrolidine-1-carboxylate (6.65 g, 12.534 mmol) in THF (50 mL) and H₂O (10 mL). The reaction was stirred at 60° C. for 2 h. Then, a second portion of iodine (300 mg, 0.0609 mL, 1.1820 mmol) was added and the reaction was stirred at 60° C. for another 2 h. The reaction was diluted with ethyl acetate (150 mL) and washed successively with aqueous 5% sodium bicarbonate solution (2×100 mL), aqueous 10% sodium thiosulfate solution (2×100 mL), and brine (100 mL). The organic phase was dried over sodium sulfate, filtered and evaporated to dryness. The resulting crude product was purified by reverse phase chromatography eluting with 5 to 100% acetonitrile in water (with 0.1% formic acid as a modifier) to give tert-butyl (4S)-4-[3-amino-4-(5-bromo-2-pyridyl)butyl]-2,2-dimethyl-pyrrolidine-1-carboxylate (4 g, 73%); ¹H NMR (400 MHz, CDCl₃) δ 8.60 (s, 1H), 7.73 (d, J=8.1 Hz, 1H), 7.07 (d, J=8.3 Hz, 1H), 3.79-3.56 (m, 1H), 3.18 (dd, J=7.5, 3.8 Hz, 1H), 2.98-2.78 (m, 2H), 2.63 (dd, J=13.7, 8.6 Hz, 1H), 2.18-2.02 (m, 1H), 1.95-1.82 (m, 1H), 1.52-1.22 (m, 22H). ESI-MS m/z calc. 425.1678, found 426.2 (M+1)⁺; Retention time: 1.45 minutes (LC method E).

### Step 3: tert-Butyl (4S)-4-[4-(5-bromo-2-pyridyl)-3-[(6-sulfamoyl-2-pyridyl)amino]butyl]-2,2-dimethyl-pyrrolidine-1-carboxylate

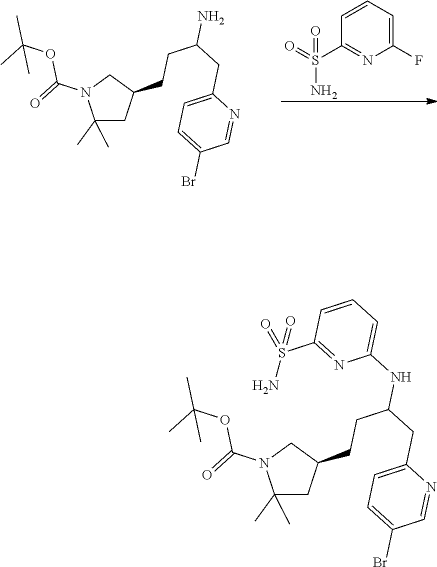 [SMILES: CC(C)(C)OC(=O)N1C[C@@H](CCC(N)Cc2ccc(Br)cn2)CC1(C)C ; NS(=O)(=O)c1cccc(F)n1 ; CC(C)(C)OC(=O)N1C[C@@H](CCC(Cc2ccc(Br)cn2)Nc2cccc(S(N)(=O)=O)n2)CC1(C)C]

To a solution of tert-butyl (4S)-4-[3-amino-4-(5-bromo-2-pyridyl)butyl]-2,2-dimethyl-pyrrolidine-1-carboxylate (3.8 g, 8.7338 mmol) and 6-fluoropyridine-2-sulfonamide (4.9 g, 27.814 mmol) in CH₃CN (28 mL) was added DIPEA (4.4520 g, 6 mL, 34.447 mmol). The reaction tube was sealed and the mixture was stirred at 120° C. for 24 h. The mixture was allowed to cooled to rt. Then, a second portion of DIPEA (2.2260 g, 3 mL, 17.223 mmol) and 6-fluoropyridine-2-sulfonamide (2.4 g, 13.623 mmol) were added to the reaction mixture. The tube was sealed and the mixture was stirred at 120° C. for 24 h. The reaction was partitioned between DCM (100 mL) and water (100 mL). The aqueous phase was extracted with DCM (2×75 mL). The combined organic layers were washed with brine (100 mL), dried over sodium sulfate, filtered and concentrated. The crude material was purified by reverse phase chromatography eluting with 5 to 100% acetonitrile in water (with 0.1% HCO₂H as a modifier). The product fractions were combined and evaporated. Ethyl acetate (250 mL) was added, and the resulting solution was washed with saturated aqueous NaHCO₃ solution (150 mL) then water (150 mL), dried over Na₂SO₄, filtered, and evaporated in vacuo to give tert-butyl (4S)-4-[4-(5-bromo-2-pyridyl)-3-[(6-sulfamoyl-2-pyridyl)amino]butyl]-2,2-dimethyl-pyrrolidine-1-carboxylate. A second purification with silica gel chromatography (eluting with 30 to 65% ethyl acetate in heptanes) afforded tert-butyl(4S)-4-[4-(5-bromo-2-pyridyl)-3-[(6-sulfamoyl-2-pyridyl)amino]butyl]-2,2-dimethyl-pyrrolidine-1-carboxylate (4.27 g, 84%) as a white solid. ¹H NMR (400 MHz, DMSO-d₆) δ 8.57 (d, J=2.4 Hz, 1H), 7.90 (dd, J=8.3, 2.4 Hz, 1H), 7.50 (t, J=7.8 Hz, 1H), 7.33 (d, J=8.3 Hz, 1H), 7.09 (s, 2H), 7.02-6.84 (m, 2H), 6.58 (d, J=8.6 Hz, 1H), 4.41-4.24 (m, 1H), 3.55-3.40 (m, 1H), 3.04-2.83 (m, 2H), 2.80-2.66 (m, 1H), 2.06-1.99 (m, 1H), 1.88-1.72 (m, 1H), 1.59-1.44 (m, 2H), 1.44-1.28 (m, 15H), 1.22-1.19 (m, 3H). ESI-MS m/z calc. 581.1671, found 582.2 (M+1)⁺; Retention time: 4.38 minutes (LC method F).

### Step 4: tert-Butyl (4S)-4-[4-(5-bromo-2-pyridyl)-3-[[6-[(6-tert-butyl-2-fluoro-pyridine-3-carbonyl)sulfamoyl]-2-pyridyl]amino]butyl]-2,2-dimethyl-pyrrolidine-1-carboxylate

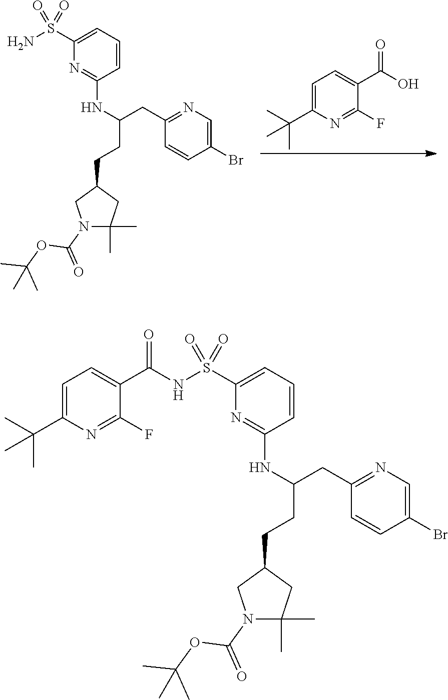

A mixture of 6-tert-butyl-2-fluoro-pyridine-3-carboxylic acid (621 mg, 3.149 mmol) and CDI (577.1 mg, 3.559 mmol) were dissolved in THF (6 mL) and the mixture was stirred at room temperature for 16 h. tert-Butyl (4S)-4-[4-(5-bromo-2-pyridyl)-3-[(6-sulfamoyl-2-pyridyl)amino]butyl]-2,2-dimethyl-pyrrolidine-1-carboxylate (1.038 g, 1.782 mmol) was added, followed by DBU (1.1 mL, 7.356 mmol), and the resulting mixture was stirred for an additional 24 h. The mixture was diluted with ethyl acetate and washed with 1 N HCl then brine, dried over sodium sulfate and concentrated in vacuo. The crude material was purified by silica gel chromatography eluting with 0-60% ethyl acetate in hexanes to give tert-butyl (4S)-4-[4-(5-bromo-2-pyridyl)-3-[[6-[(6-tert-butyl-2-fluoro-pyridine-3-carbonyl)sulfamoyl]-2-pyridyl]amino]butyl]-2,2-dimethyl-pyrrolidine-1-carboxylate (557 mg, 41%) as a clear oil. ESI-MS m/z calc. 760.24176, found 761.3 (M+1)⁺; Retention time: 0.87 minutes (LC method D).

### Step 5: (14S)-17-[(5-bromopyridin-2-yl)methyl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione and (14S)-17-[(5-bromopyridin-2-yl)methyl]-8-tert-butyl-12,12-dimethyl-2λ⁶-thia-3,9,11,18,23-pentaazatetracyclo [17.3.1.111,14.05,10]tetracosa-1(23),5(10),6,8,19,21-hexaene-2,2,4-trione

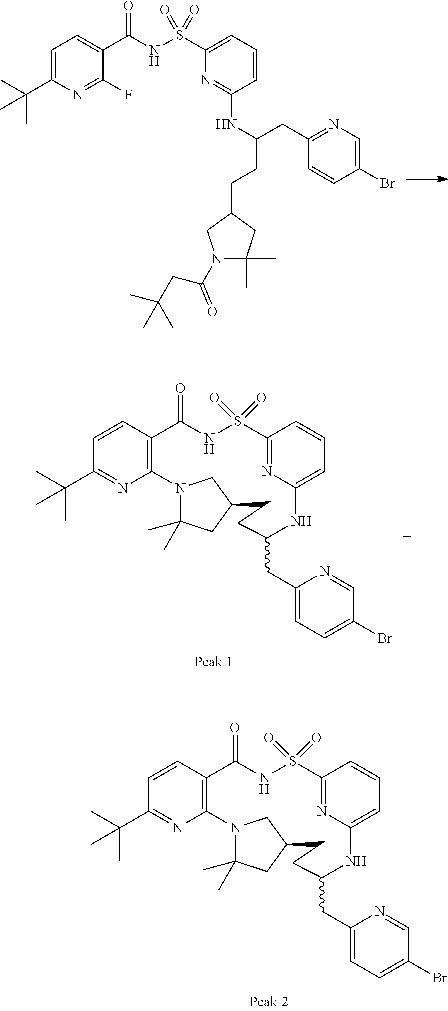 [SMILES: CC(C)(C)CC(=O)N1CC(CCC(Cc2ccc(Br)cn2)Nc2cccc(S(=O)(=O)NC(=O)c3ccc(C(C)(C)C)nc3F)n2)CC1(C)C ; CC(C)(C)c1ccc2c(n1)N1C[C@@H](CCC(Cc3ccc(Br)cn3)Nc3cccc(n3)S(=O)(=O)NC2=O)CC1(C)C ; CC(C)(C)c1ccc2c(n1)N1C[C@@H](CCC(Cc3ccc(Br)cn3)Nc3cccc(n3)S(=O)(=O)NC2=O)CC1(C)C]

Stage 1: tert-Butyl (4S)-4-[4-(5-bromo-2-pyridyl)-3-[[6-[(6-tert-butyl-2-fluoro-pyridine-3-carbonyl)sulfamoyl]-2-pyridyl]amino]butyl]-2,2-dimethyl-pyrrolidine-1-carboxylate (557 mg, 0.7312 mmol) and TFA (600 μL, 7.788 mmol) were combined in DCM (5 mL) and stirred at room temperature for 2 h. The reaction mixture was evaporated and used directly in the next step.

Stage 2: Amine from Stage 1 was dissolved in NMP (10 mL) with potassium carbonate (1.21 g, 8.755 mmol) and heated at 120° C. for 16 h. The reaction was cooled to room temperature and partitioned between ethyl acetate and 1 N HCl. The organics were separated, washed with brine, dried over sodium sulfate and evaporated in vacuo. The crude material was purified by silica gel chromatography eluting with 0-100% ethyl acetate in hexanes to give two diastereomers.

Peak 1: (14S)-17-[(5-bromopyridin-2-yl)methyl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (99.7 mg, 21%); ESI-MS m/z calc. 640.1831, found 641.3 (M+1)⁺; Retention time: 0.79 minutes (LC method D).

Peak 2: (14S)-17-[(5-bromopyridin-2-yl)methyl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (120.0 mg, 26%); ESI-MS m/z calc. 640.1831, found 641.3 (M+1)⁺; Retention time: 0.76 minutes (LC method D).

### Step 6: (14S)-8-tert-Butyl-17-({5-[(1E)-3,3-dimethylbut-1-en-1-yl]pyridin-2-yl}methyl)-12,12-dimethyl-2λ⁶-thia-3,9,11,18,23-pentaazatetracyclo [17.3.1.111,14.05,10]tetracosa-1(23),5(10),6,8,19,21-hexaene-2,2,4-trione

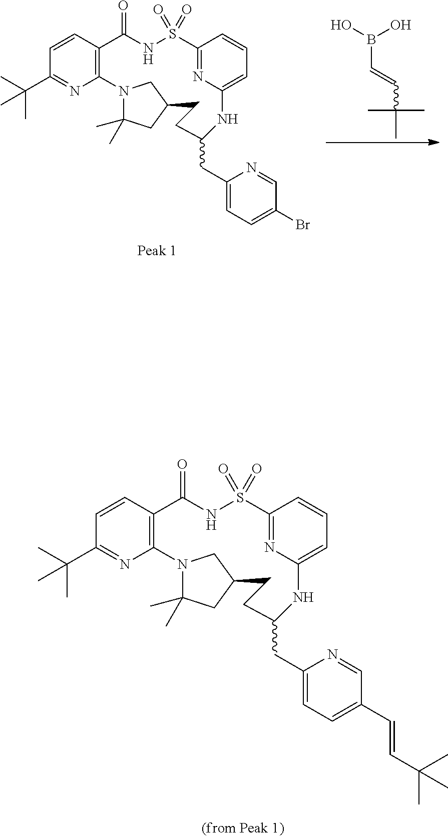

Peak 1 (14S)-17-[(5-Bromopyridin-2-yl)methyl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (30.1 mg, 0.04691 mmol), [(E)-3,3-dimethylbut-1-enyl]boronic acid (12.7 mg, 0.09924 mmol), dichloro[1,1′-bis(diphenylphosphino)ferrocene]palladium (II) dichloromethane adduct (6 mg, 0.007347 mmol), and aqueous potassium carbonate (70 μL, of 2 M, 0.1400 mmol) were combined in DMF (0.5 mL) and heated at 90° C. for 16 h. The reaction was cooled, diluted with DMSO (0.5 mL), filtered and purified by reverse-phase HPLC utilizing a gradient of 1-99% acetonitrile in 5 mM HCl to yield Peak 1 (14S)-8-tert-butyl-17-({5-[(1E)-3,3-dimethylbut-1-en-1-yl]pyridin-2-yl }methyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (21.4 mg, 67%) as an off-white solid. ESI-MS m/z calc. 644.3508, found 645.5 (M+1)⁺; Retention time: 0.7 minutes (LC method D).

### Step 7: (14S)-8-tert-Butyl-17-{[5-(3,3-dimethylbutyl)pyridin-2-yl]methyl}-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 420)

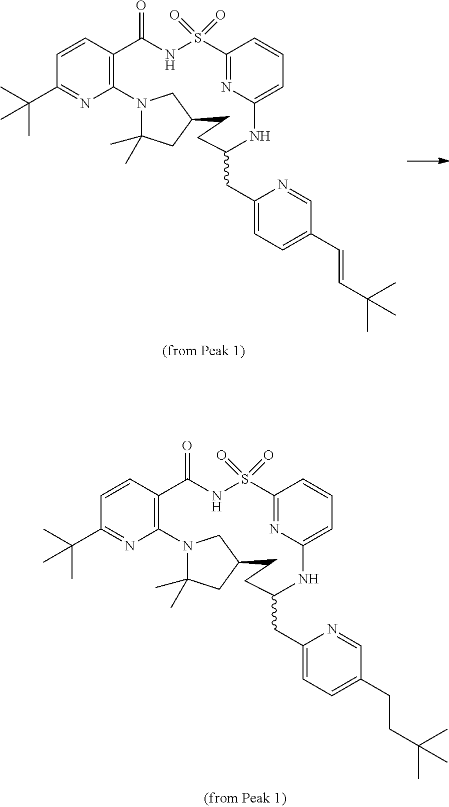

Peak 1 (14S)-8-tert-Butyl-17-({5-[(1E)-3,3-dimethylbut-1-en-1-yl]pyridin-2-yl}methyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (21.4 mg, 0.03141 mmol) and Pd/C (8.9 mg of 10% w/w, 0.008363 mmol) were combined in methanol (4 mL) under a balloon of hydrogen. The reaction mixture was stirred at room temperature for 4 h. The reaction was filtered and the solids were washed with methanol. The organics were evaporated and the crude material was purified by reverse-phase HPLC utilizing a gradient of 1-99% acetonitrile in 5 mM HCl to yield Peak 1 (14S)-8-tert-butyl-17-{[5-(3,3-dimethylbutyl)pyridin-2-yl]methyl}-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (6.0 mg, 28%) as a white solid. ESI-MS m/z calc. 646.3665, found 647.5 (M+1)⁺; Retention time: 1.86 minutes (LC method A).

### Step 8: (14S)-8-tert-Butyl-17-({5-[(1E)-3,3-dimethylbut-1-en-1-yl]pyridin-2-yl}methyl)-12,12-dimethyl-2λ⁶-thia-3,9,11,18,23-pentaazatetracyclo [17.3.1.111,14.05,10]tetracosa-1(23),5(10),6,8,19,21-hexaene-2,2,4-trione

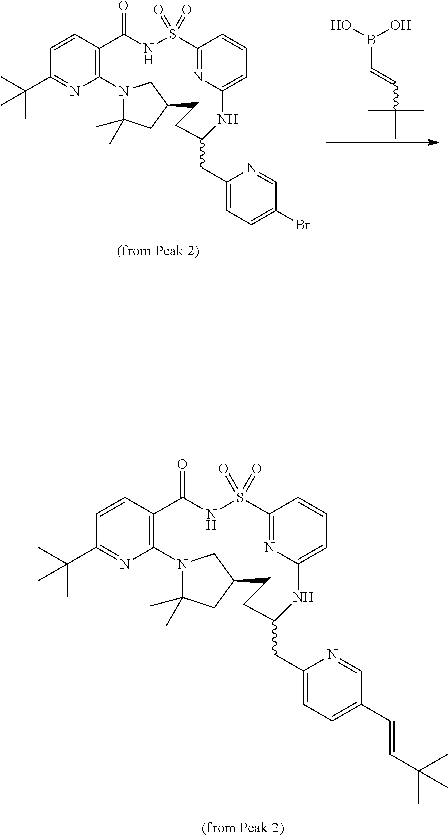

Peak 2 (14S)-17-[(5-bromopyridin-2-yl)methyl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (31.7 mg, 0.04941 mmol), [(E)-3,3-dimethylbut-1-enyl]boronic acid (13.3 mg, 0.1039 mmol), dichloro[1,1′-bis(diphenylphosphino)ferrocene]palladium (II) dichloromethane adduct (4.5 mg, 0.005510 mmol), and 2M aqueous potassium carbonate (75 μL of 2 M, 0.1500 mmol) were combined in DMF (0.5 mL) and heated at 90° C. for 16 h. The reaction mixture was diluted with DMSO (0.5 mL), filtered, and purified by reverse-phase HPLC utilizing a gradient of 1-99% acetonitrile in 5 mM HCl to yield Peak 2 (14S)-8-tert-butyl-17-({5-[(1E)-3,3-dimethylbut-1-en-1-yl]pyridin-2-yl}methyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (19.2 mg, 57%) as an off-white solid. ESI-MS m/z calc. 644.3508, found 645.5 (M+1)⁺; Retention time: 0.73 minutes (LC method D).

### Step 9: (14S)-8-tert-Butyl-17-{[5-(3,3-dimethylbutyl)pyridin-2-yl]methyl}-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 421)

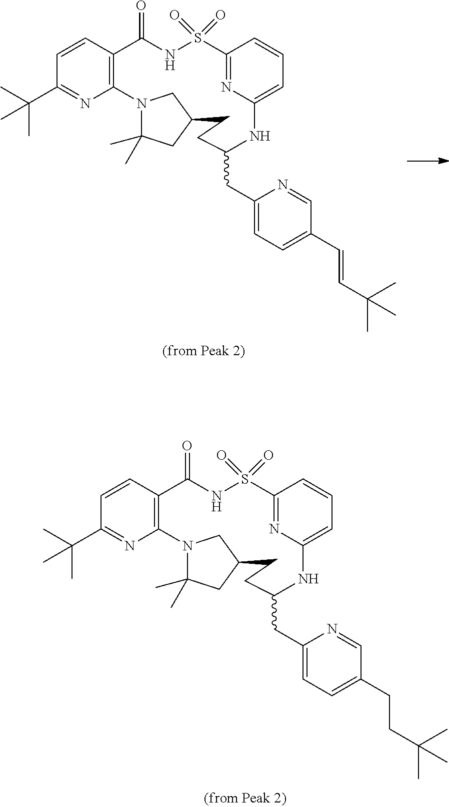

Peak 2 (14S)-8-tert-butyl-17-({5-[(1E)-3,3-dimethylbut-1-en-1-yl]pyridin-2-yl}methyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (19.2 mg, 0.02818 mmol) and Pd/C (8.1 mg of 10% w/w, 0.007611 mmol) were combined in methanol (4 mL) under a balloon of hydrogen. The reaction mixture was stirred at room temperature for 4 h. The reaction was filtered and the solids were washed with methanol. The organics were evaporated and the crude material was purified by reverse-phase HPLC utilizing a gradient of 1-99% acetonitrile in 5 mM HCl to yield (14S)-8-tert-butyl-17-{[5-(3,3-dimethylbutyl)pyridin-2-yl]methyl}-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Hydrochloride salt) (9.9 mg, 51%) as a white solid. ESI-MS m/z calc. 646.3665, found 647.5 (M+1)⁺; Retention time: 1.91 minutes (LC method A).

### Example 156: Preparation of (14S)-8-tert-Butyl-17-({5-[(3-methoxypropyl)(methyl)amino]pyridin-2-yl}methyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 422)

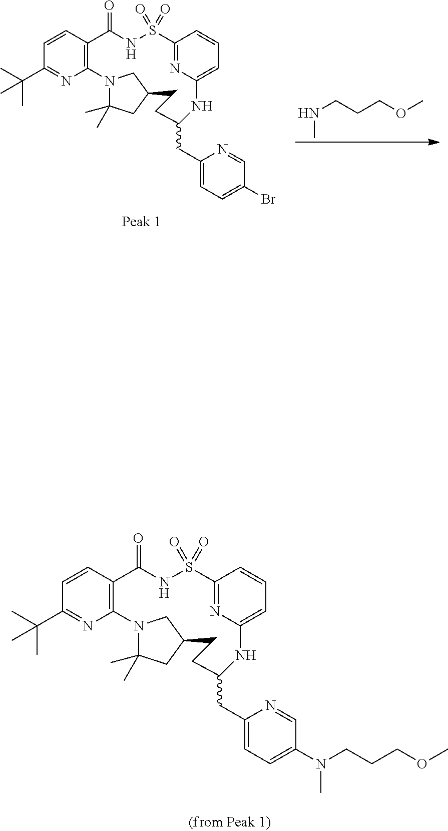

Peak 1 (14S)-17-[(5-bromopyridin-2-yl)methyl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (27.9 mg, 0.04348 mmol) and 3-methoxy-N-methyl-propan-1-amine (26.1 mg, 0.2530 mmol) were combined in dioxane (400 μL). RuPhos Pd G3 (5.4 mg, 0.006456 mmol) and sodium tert-butoxide (22.6 mg, 0.2352 mmol) were added. The resulting mixture was sparged with nitrogen gas, then stirred at 90° C. for 6 h. It was then cooled to room temperature, diluted with DMSO (600 μL), filtered, and purified by reverse-phase HPLC utilizing a gradient of 1-99% acetonitrile in 5 mM HCl to yield Peak 1 (14S)-8-tert-butyl-17-({5-[(3-methoxypropyl)(methyl)amino]pyridin-2-yl}methyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (4.7 mg, 15%). ESI-MS m/z calc. 663.3567, found 664.6 (M+1)⁺; Retention time: 1.68 minutes (LC method A).

### Example 157: Preparation of (14S)-8-tert-Butyl-17-({5-[(3-methoxypropyl)(methyl)amino]pyridin-2-yl}methyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 423)

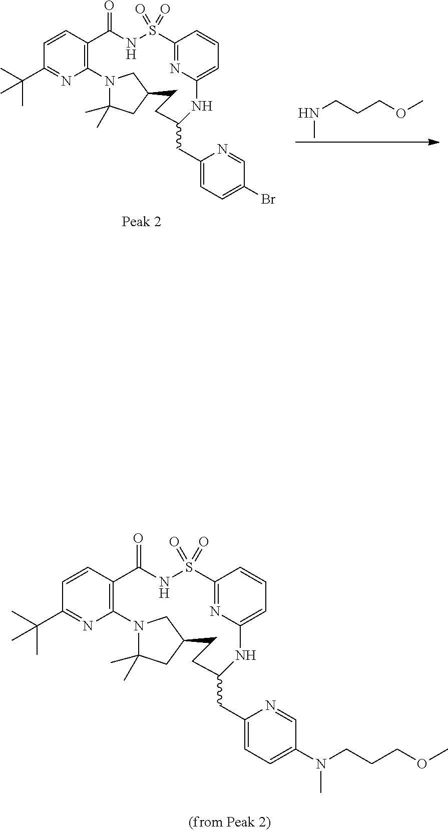

Peak 2 (14S)-17-[(5-bromopyridin-2-yl)methyl]-8-tert-butyl-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (26.5 mg, 0.04130 mmol) and 3-methoxy-N-methyl-propan-1-amine (27.0 mg, 0.2617 mmol) were combined in dioxane (400 μL). RuPhos Pd G3 (5.3 mg, 0.006337 mmol) and sodium tert-butoxide (23.1 mg, 0.2404 mmol) were added. The resulting mixture was sparged with nitrogen gas, then stirred at 90° C. for 6 h. It was then cooled to room temperature, diluted with DMSO (600 μL), filtered, and purified by reverse-phase HPLC utilizing a gradient of 1-99% acetonitrile in 5 mM HCl to yield (14S)-8-tert-butyl-17-({5-[(3-methoxypropyl)(methyl)amino]pyridin-2-yl}methyl)-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (hydrochloride salt) (2.8 mg, 10%). ESI-MS m/z calc. 663.3567, found 664.5 (M+1)⁺; Retention time: 1.75 minutes (LC method A).

### Example 158: Preparation of (14S)-8-tert-Butyl-16-(2,2-dimethylpropyl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 424), (14S)-8-tert-Butyl-16-(2,2-dimethylpropyl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 425), and (14S)-8-tert-butyl-16-(2,2-dimethylpropyl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 426)

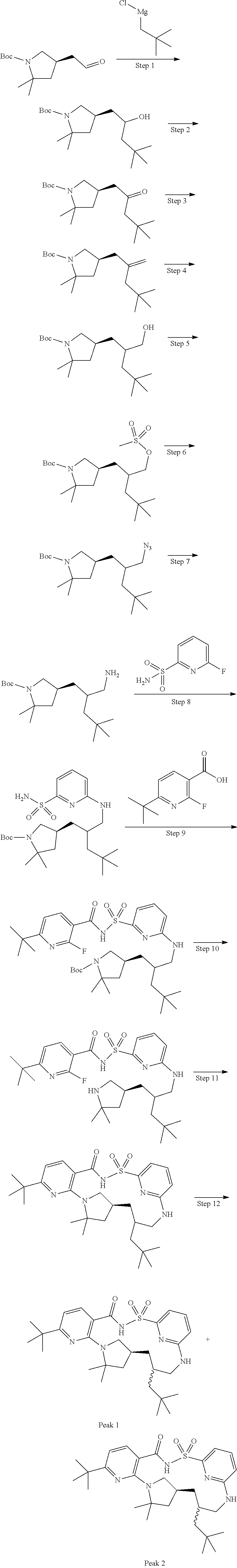

### Step 1: tert-Butyl (4R)-4-(2-hydroxy-4,4-dimethyl-pentyl)-2,2-dimethyl-pyrrolidine-1-carboxylate

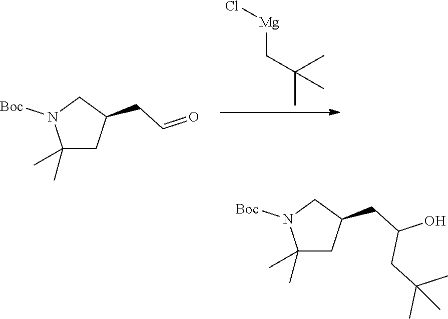

To a solution of tert-butyl (4R)-2,2-dimethyl-4-(2-oxoethyl)pyrrolidine-1-carboxylate (1.68 g, 6.3071 mmol) in THF (27 mL) at 0° C. was added dropwise a solution of neopentylmagnesium chloride in THF (14 mL of 1 M, 14.0 mmol). The mixture was stirred at 0° C. for 1 h, then quenched with saturated ammonium chloride aqueous solution (30 mL). The mixture was diluted in ethyl acetate (100 mL) and the phases were separated. The aqueous phase was extracted with ethyl acetate (2×50 mL). The combined organic layers were dried over anhydrous sodium sulfate, filtered and concentrated under reduced pressure. The crude residue was purified by silica gel chromatography eluting from 0% to 50% ethyl acetate in heptanes to afford tert-butyl (4R)-4-(2-hydroxy-4,4-dimethyl-pentyl)-2,2-dimethyl-pyrrolidine-1-carboxylate (1.403 g, 64%) as a white solid. ¹H NMR (400 MHz, CDCl₃) δ 3.86-3.64 (m, 2H), 2.98-2.87 (m, 1H), 2.37-2.26 (m, 1H), 2.03-1.89 (m, 1H), 1.53-1.44 (m, 12H), 1.43-1.29 (m, 9H), 0.99-0.97 (m, 9H). ESI-MS m/z calc. 313.2617, found 258.2 (M-tBu+2H=M−55)⁺; Retention time: 2.07 minutes (LC method E).

### Step 2: tert-Butyl (4R)-4-(4,4-dimethyl-2-oxo-pentyl)-2,2-dimethyl-pyrrolidine-1-carboxylate

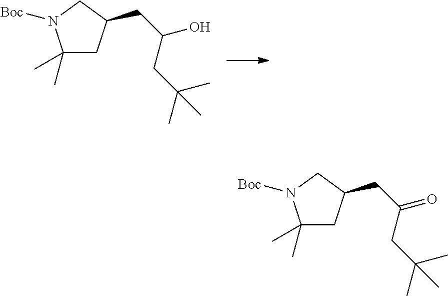 [SMILES: CC(C)(C)CC(O)C[C@@H]1CN(C(=O)OC(C)(C)C)C(C)(C)C1 ; CC(C)(C)CC(=O)C[C@@H]1CN(C(=O)OC(C)(C)C)C(C)(C)C1]

To a solution of tert-butyl (4R)-4-(2-hydroxy-4,4-dimethyl-pentyl)-2,2-dimethyl-pyrrolidine-1-carboxylate (1.18 g, 3.3878 mmol) in DCM (12 mL) was added DessMartin periodinane (1.87 g, 4.4089 mmol) and the mixture was stirred for 30 min. The mixture was quenched with saturated aqueous NaHCO₃ solution (40 mL) and extracted with DCM (2×40 mL). The organic phase was washed with saturated aqueous NaHCO₃ solution (20 mL) and brine (20 mL), dried over sodium sulfate, filtered, and concentrated to dryness. The crude material was purified by silica gel chromatography eluting with 0 to 40% ethyl acetate in heptanes to give tert-butyl (4R)-4-(4,4-dimethyl-2-oxo-pentyl)-2,2-dimethyl-pyrrolidine-1-carboxylate (997 mg, 93%) as a white solid. ESI-MS m/z calc. 311.246, found 256.2 (M-tBu+2H=M−55)⁺; Retention time: 2.11 minutes (LC method E).

### Step 3: tert-Butyl (4S)-4-(4,4-dimethyl-2-methylene-pentyl)-2,2-dimethyl-pyrrolidine-1-carboxylate

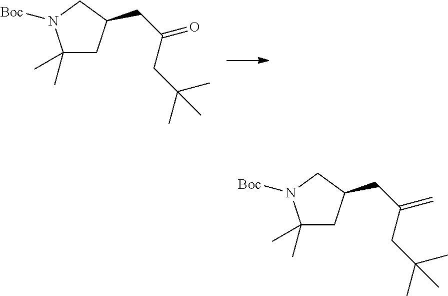 [SMILES: CC(C)(C)CC(=O)C[C@@H]1CN(C(=O)OC(C)(C)C)C(C)(C)C1 ; C=C(C[C@@H]1CN(C(=O)OC(C)(C)C)C(C)(C)C1)CC(C)(C)C]

To a solution of methyl(triphenyl)phosphonium bromide (1.64 g, 4.5910 mmol) in THF (8.5 mL) at 0° C. was added dropwise a solution of NaHMDS in THF (4.6 mL of 1 M, 4.6000 mmol), and the mixture was stirred at room temperature for 1 h. The resulting yellow solution was treated with tert-butyl (4R)-4-(4,4-dimethyl-2-oxo-pentyl)-2,2-dimethyl-pyrrolidine-1-carboxylate (580 mg, 1.8268 mmol) in THF (8.5 mL), and this was stirred at 70° C. for 16 h. After cooling to room temperature, water (30 mL) was added, and the mixture was extracted with ethyl acetate (3×30 mL). The combined organic phase was washed with brine (2×20 mL), dried over sodium sulfate, filtered and concentrated to dryness. The crude residue was purified by silica gel chromatography eluting with 0% to 20% ethyl acetate in heptanes to give tert-butyl (4S)-4-(4,4-dimethyl-2-methylene-pentyl)-2,2-dimethyl-pyrrolidine-1-carboxylate (444 mg, 77%) as a colorless oil. ESI-MS m/z calc. 309.2668, found 254.2 (M-tBu+2H=M−55)⁺; Retention time: 2.51 minutes (LC method E).

### Step 4: tert-Butyl (4S)-4-[2-(hydroxymethyl)-4,4-dimethyl-pentyl]-2,2-dimethyl-pyrrolidine-1-carboxylate

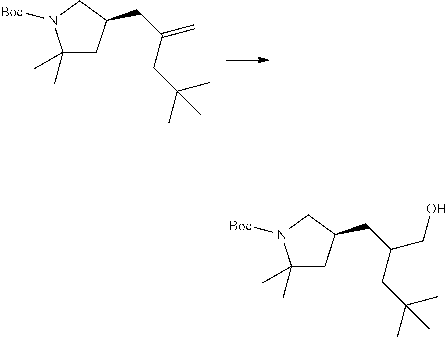 [SMILES: C=C(C[C@@H]1CN(C(=O)OC(C)(C)C)C(C)(C)C1)CC(C)(C)C ; CC(C)(C)CC(CO)C[C@@H]1CN(C(=O)OC(C)(C)C)C(C)(C)C1]

A solution of tert-butyl (4S)-4-(4,4-dimethyl-2-methylene-pentyl)-2,2-dimethyl-pyrrolidine-1-carboxylate (444 mg, 1.3629 mmol) in THF (15 mL) was cooled to 0° C., and 9-borabicyclo[3.3.1]nonane solution in THF (9.5 mL of 0.5 M, 4.7500 mmol) was added. The resulting mixture was stirred at room temperature under nitrogen for 16 h. After cooling to 0° C., 1 N NaOH (6.9 mL, 6.9000 mmol) and hydrogen peroxide (666 mg, 0.6000 mL, 6.8529 mmol) were slowly added, and the mixture was stirred at room temperature for 24 h. The mixture was diluted with water (30 mL) and extracted with ethyl acetate (3×40 mL). The organic phase was dried over sodium sulfate, filtered and concentrated to dryness. The crude oil was purified by silica gel chromatography using a gradient of 0% to 20% ethyl acetate in heptanes to give tert-butyl (4S)-4-[2-(hydroxymethyl)-4,4-dimethyl-pentyl]-2,2-dimethyl-pyrrolidine-1-carboxylate (440 mg, 89%) as a colorless oil. ESI-MS m/z calc. 327.2773, found 272.2 (M-tBu+2H=M−55)⁺; Retention time: 2.08 minutes (LC method E).

### Step 5: tert-Butyl (4S)-4-[4,4-dimethyl-2-(methylsulfonyloxymethyl)pentyl]-2,2-dimethyl-pyrrolidine-1-carboxylate

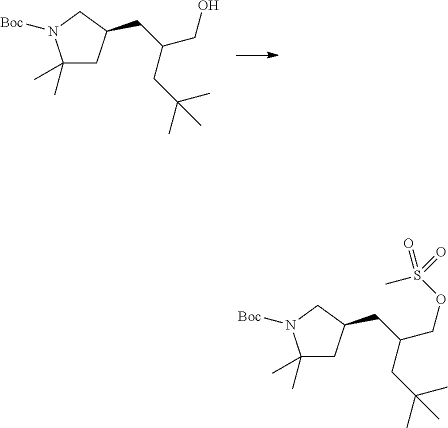 [SMILES: CC(C)(C)CC(CO)C[C@@H]1CN(C(=O)OC(C)(C)C)C(C)(C)C1 ; CC(C)(C)CC(COS(C)(=O)=O)C[C@@H]1CN(C(=O)OC(C)(C)C)C(C)(C)C1]

To a solution of tert-butyl (4S)-4-[2-(hydroxymethyl)-4,4-dimethyl-pentyl]-2,2-dimethyl-pyrrolidine-1-carboxylate (440 mg, 1.2092 mmol) in DCM (6.5 mL) was added at 0° C. triethylamine (159.72 mg, 0.22 mL, 1.5784 mmol), followed by methanesulfonyl chloride (165.76 mg, 0.112 mL, 1.4470 mmol). The ice bath was removed, and the mixture was stirred for 2.5 h. The mixture was partitioned between DCM (25 mL) and water (20 mL). The phases were separated, and the aqueous layer was extracted with DCM (2×20 mL). The organic phase was washed with brine (2×20 mL), dried over sodium sulfate, filtered and concentrated to dryness to afford crude tert-butyl (4S)-4-[4,4-dimethyl-2-(methylsulfonyloxymethyl)pentyl]-2,2-dimethyl-pyrrolidine-1-carboxylate (540 mg, 101%) as a colorless oil. ESI-MS m/z calc. 405.25488, found 350.2 (M-tBu+2H=M−55)⁺; Retention time: 2.09 minutes (LC method E).

### Step 6: tert-Butyl (4S)-4-[2-(azidomethyl)-4,4-dimethyl-pentyl]-2,2-dimethyl-pyrrolidine-1-carboxylate

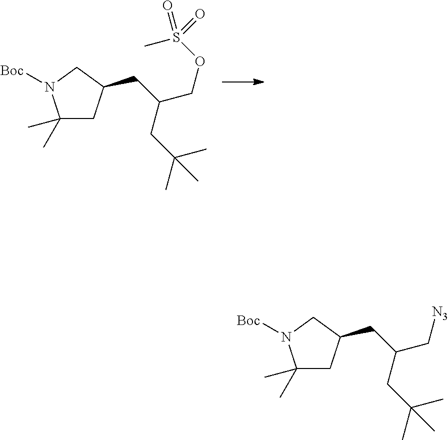 [SMILES: CC(C)(C)CC(COS(C)(=O)=O)C[C@@H]1CN(C(=O)OC(C)(C)C)C(C)(C)C1 ; CC(C)(C)CC(CN=[N+]=[N-])C[C@@H]1CN(C(=O)OC(C)(C)C)C(C)(C)C1]

To a solution of tert-butyl (4S)-4-[4,4-dimethyl-2-(methylsulfonyloxymethyl)pentyl]-2,2-dimethyl-pyrrolidine-1-carboxylate (540 mg, 1.2169 mmol) in DMF (8.5 mL) was added sodium azide (175 mg, 2.6919 mmol) and the mixture was stirred for 16 h at 65° C. The mixture was diluted with water (25 mL) and extracted with ethyl acetate (2×25 mL). The organic phase was washed with brine (3×25 mL), dried over sodium sulfate, filtered and concentrated to dryness to give crude tert-butyl (4S)-4-[2-(azidomethyl)-4,4-dimethyl-pentyl]-2,2-dimethyl-pyrrolidine-1-carboxylate (446 mg, 95%) as a colorless oil. ESI-MS m/z calc. 352.2838, found 253.4 (M-Boc+2H=M−99)⁺; Retention time: 2.41 minutes (LC method E).

### Step 7: tert-Butyl (4S)-4-[2-(aminomethyl)-4,4-dimethyl-pentyl]-2,2-dimethyl-pyrrolidine-1-carboxylate

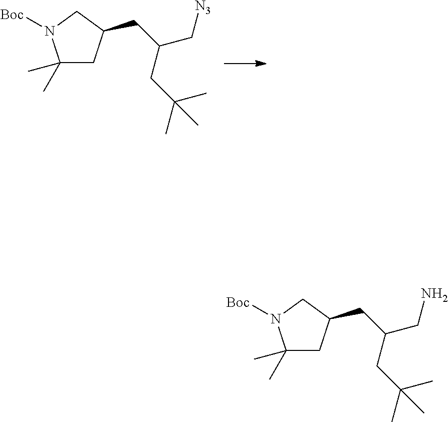 [SMILES: CC(C)(C)CC(CN=[N+]=[N-])C[C@@H]1CN(C(=O)OC(C)(C)C)C(C)(C)C1 ; CC(C)(C)CC(CN)C[C@@H]1CN(C(=O)OC(C)(C)C)C(C)(C)C1]

A solution of tert-butyl (4S)-4-[2-(azidomethyl)-4,4-dimethyl-pentyl]-2,2-dimethyl-pyrrolidine-1-carboxylate (446 mg, 1.1513 mmol) in methanol (11 mL) was degassed by bubbling nitrogen for 10 min. To the solution was added 10% palladium on carbon (50% wet; 230 mg, 0.1081 mmol) and the mixture was degassed with nitrogen for an additional 10 min. Hydrogen was bubbled through the mixture for 10 min, and then the mixture was stirred at room temperature under hydrogen atmosphere for 4 h. The mixture was filtered through a Celite pad and rinsed with methanol (25 mL). The filtrate was concentrated under reduced pressure to give crude tert-butyl (4S)-4-[2-(aminomethyl)-4,4-dimethyl-pentyl]-2,2-dimethyl-pyrrolidine-1-carboxylate (349 mg, 88%) as a colorless oil. ¹H NMR (400 MHz, CDCl₃) δ 3.78-3.59 (m, 1H), 2.98-2.82 (m, 1H), 2.66-2.54 (m, 1H), 2.21-2.12 (m, 1H), 1.95-1.88 (m, 1H), 1.75-1.66 (m, 2H), 1.53-1.34 (m, 16H), 1.32-1.22 (m, 6H), 0.91-0.88 (m, 9H). ESI-MS m/z calc. 326.29333, found 327.4 (M+1)⁺; Retention time: 1.51 minutes (LC method E).

### Step 8: tert-Butyl (4S)-4-[4,4-dimethyl-2-[[(6-sulfamoyl-2-pyridyl)amino]methyl]pentyl]-2,2-dimethyl-pyrrolidine-1-carboxylate

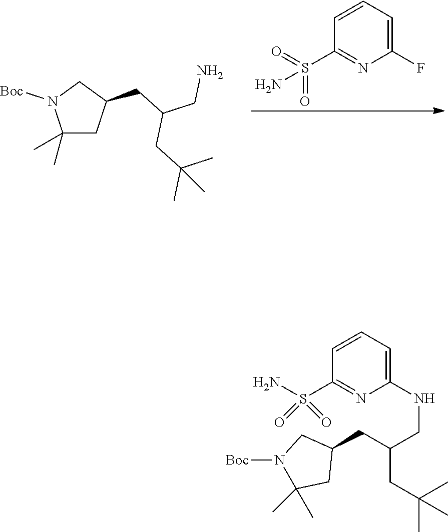 [SMILES: CC(C)(C)CC(CN)C[C@@H]1CN(C(=O)OC(C)(C)C)C(C)(C)C1 ; NS(=O)(=O)c1cccc(F)n1 ; CC(C)(C)CC(CNc1cccc(S(N)(=O)=O)n1)C[C@@H]1CN(C(=O)OC(C)(C)C)C(C)(C)C1]

To a solution of tert-butyl (4S)-4-[2-(aminomethyl)-4,4-dimethyl-pentyl]-2,2-dimethyl-pyrrolidine-1-carboxylate (82 mg, 0.2509 mmol) in MeCN (1 mL) was added DIPEA (129.85 mg, 0.175 mL, 1.0047 mmol) and 6-fluoropyridine-2-sulfonamide (445 mg, 2.5260 mmol). The reaction vial was sealed and the mixture was heated at 120° C. for 18 h. The resulting mixture was cooled to room temperature, filtered, and purified by reverse phase chromatography eluting with 5% to 100% methanol in water to give tert-butyl (4S)-4-[4,4-dimethyl-2-[[(6-sulfamoyl-2-pyridyl)amino]methyl]pentyl]-2,2-dimethyl-pyrrolidine-1-carboxylate (75 mg, 62%) as a white solid. ESI-MS m/z calc. 482.29266, found 427.2 (M-tBu+2H=M−55)⁺; Retention time: 2.03 minutes (LC method E).

### Step 9: tert-Butyl (4S)-4-[2-[[[6-[(6-tert-butyl-2-fluoro-pyridine-3-carbonyl)sulfamoyl]-2-pyridyl]amino]methyl]-4,4-dimethyl-pentyl]-2,2-dimethyl-pyrrolidine-1-carboxylate

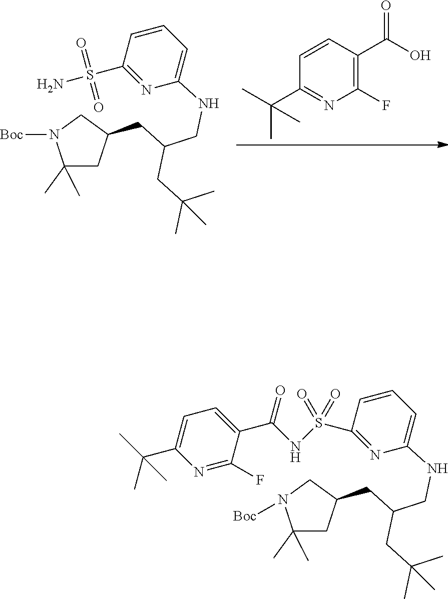

A round-bottomed flask was charged with 6-tert-butyl-2-fluoro-pyridine-3-carboxylic acid (50 mg, 0.2535 mmol) and THF (1.5 mL). 1,1′-Carbonyldiimidazole (41 mg, 0.2529 mmol) was added and the mixture was stirred at room temperature for 2 h. In a separate flask, a solution of tert-butyl (4S)-4-[4,4-dimethyl-2-[[(6-sulfamoyl-2-pyridyl)amino]methyl]pentyl]-2,2-dimethyl-pyrrolidine-1-carboxylate (75 mg, 0.1554 mmol) in THF (1 mL) was prepared under nitrogen and it was added into the activated acid solution. Then, 1,8-Diazabicyclo[5.4.0]undec-7-ene (71.260 mg, 70 μL, 0.4681 mmol) was added, and the reaction mixture was stirred at room temperature under nitrogen for 16 h. After concentration under reduced pressure, the crude material was purified by reverse phase chromatography, eluting with a gradient of 5% to 100% of methanol in water to give tert-butyl (4S)-4-[2-[[[6-[(6-tert-butyl-2-fluoro-pyridine-3-carbonyl)sulfamoyl]-2-pyridyl]amino]methyl]-4,4-dimethyl-pentyl]-2,2-dimethyl-pyrrolidine-1-carboxylate (95 mg, 79%) as a white solid. ¹H NMR (400 MHz, CDCl₃) δ 10.71 (br s, 1H), 8.34 (dd, J=9.8, 8.8 Hz, 1H), 7.56-7.49 (m, 1H), 7.46-7.41 (m, 1H), 7.13 (dd, J=7.8, 2.0 Hz, 1H), 6.43-6.36 (m, 1H), 4.79-4.67 (m, 1H), 3.50-3.42 (m, 4H), 2.96-2.88 (m, 2H), 2.25-2.14 (m, 1H), 2.09-2.00 (m, 2H), 1.49-1.42 (m, 10H), 1.35-1.26 (m, 17H), 0.90 (br s, 9H). ESI-MS m/z calc. 661.3673, found 662.2 (M+1)⁺; Retention time: 2.37 minutes (LC method E).

### Step 10: 6-tert-Butyl-N-[[6-[[2-[[(3S)-5,5-dimethylpyrrolidin-3-yl]methyl]-4,4-dimethyl-pentyl]amino]-2-pyridyl]sulfonyl]-2-fluoro-pyridine-3-carboxamide

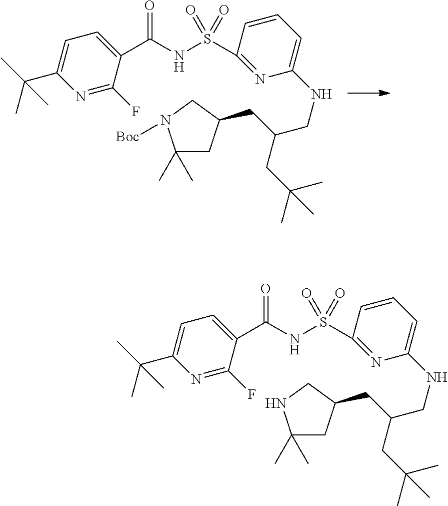

A solution of tert-butyl (4S)-4-[2-[[[6-[(6-tert-butyl-2-fluoro-pyridine-3-carbonyl)sulfamoyl]-2-pyridyl]amino]methyl]-4,4-dimethyl-pentyl]-2,2-dimethyl-pyrrolidine-1-carboxylate (95 mg, 0.1435 mmol) in DCM (1 mL) was treated with 4 M HCl in dioxane (0.4 mL, 1.6 mmol). After stirring for 2 h at room temperature, the mixture was concentrated to dryness to give crude 6-tert-butyl-N-[[6-[[2-[[(3S)-5,5-dimethylpyrrolidin-3-yl]methyl]-4,4-dimethyl-pentyl]amino]-2-pyridyl]sulfonyl]-2-fluoro-pyridine-3-carboxamide (hydrochloride salt) (103 mg, 91%) as a white solid. ¹H NMR (400 MHz, DMSO-d₆) δ 12.48 (br s, 1H), 9.67 (s, 1H), 8.89-8.76 (m, 1H), 8.18-8.10 (m, 1H), 7.64-7.57 (m, 1H), 7.50 (s, 1H), 7.25-7.17 (m, 1H), 7.16 (d, J=1.7 Hz, 1H), 6.81-6.73 (m, 1H), 3.51-3.43 (m, 2H), 3.28-3.21 (m, 2H), 2.87-2.72 (m, 1H), 2.71-2.62 (m, 2H), 2.11-2.02 (m, 1H), 1.97-1.87 (m, 2H), 1.39-1.33 (m, 3H), 1.32-1.25 (m, 14H), 0.80 (s, 9H). ESI-MS m/z calc. 561.3149, found 562.2 (M+1)⁺; Retention time: 1.62 minutes (LC method E).

### Step 11: (14S)-8-tert-Butyl-16-(2,2-dimethylpropyl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 424)

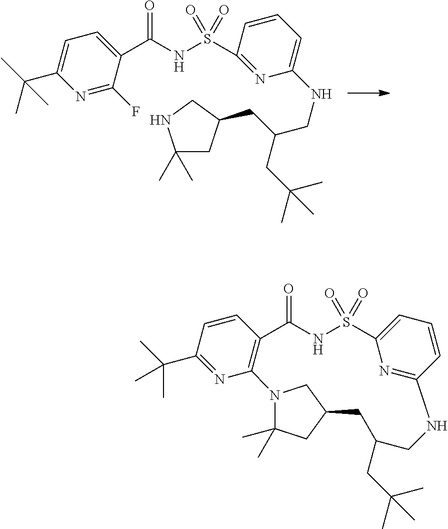

To a solution of 6-tert-butyl-N-[[6-[[2-[[(3S)-5,5-dimethylpyrrolidin-3-yl]methyl]-4,4-dimethyl-pentyl]amino]-2-pyridyl]sulfonyl]-2-fluoro-pyridine-3-carboxamide (hydrochloride salt) (85 mg, 0.1421 mmol) in DMSO (1.5 mL) was added potassium carbonate (150 mg, 1.0853 mmol). The reaction vial was sealed, and the mixture was stirred at 120° C. for 24 h. The mixture was cooled to room temperature, filtered and purified by reverse phase chromatography eluting with 5% to 100% methanol in water to give (14S)-8-tert-butyl-16-(2,2-dimethylpropyl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (21 mg, 27%) as a white solid. ¹H NMR (400 MHz, DMSO-d₆) δ 7.51-7.29 (m, 2H), 6.91-6.82 (m, 1H), 6.65-6.48 (m, 1H), 6.47-6.31 (m, 2H), 3.95-3.80 (m, 1H), 3.57-3.37 (m, 1H), 3.05-2.92 (m, 1H), 2.76-2.65 (m, 1H), 2.30-2.09 (m, 1H), 1.94-1.76 (m, 1H), 1.70 (dd, J=11.2, 4.4 Hz, 1H), 1.62 (s, 1H), 1.57 (s, 2H), 1.48 (s, 3H), 1.45-1.35 (m, 2H), 1.34-1.16 (m, 12H), 0.97-0.84 (m, 9H). ESI-MS m/z calc. 541.30865, found 542.3 (M+1)⁺; Retention time: 4.06 minutes (LC method F).

### Step 12: (14S)-8-tert-Butyl-16-(2,2-dimethylpropyl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 425) and (14S)-8-tert-butyl-16-(2,2-dimethylpropyl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 426)

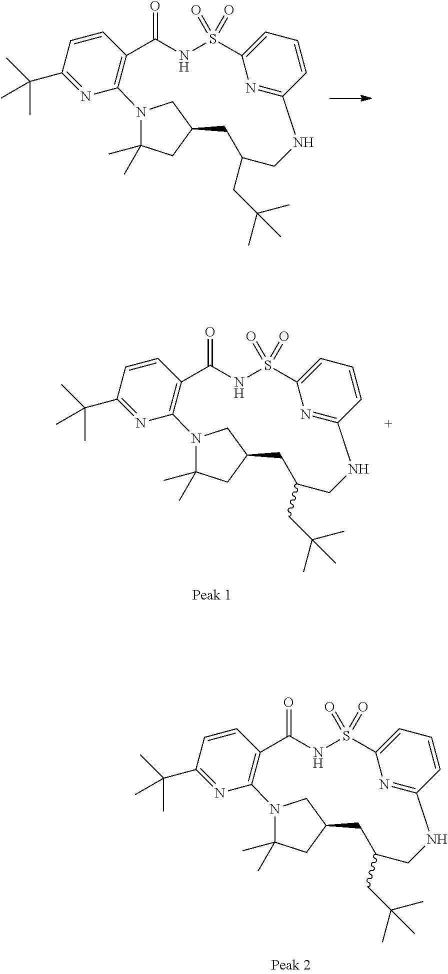 [SMILES: CC(C)(C)CC1CNc2cccc(n2)S(=O)(=O)NC(=O)c2ccc(C(C)(C)C)nc2N2C[C@@H](C1)CC2(C)C ; CC(C)(C)CC1CNc2cccc(n2)S(=O)(=O)NC(=O)c2ccc(C(C)(C)C)nc2N2C[C@@H](C1)CC2(C)C ; CC(C)(C)CC1CNc2cccc(n2)S(=O)(=O)NC(=O)c2ccc(C(C)(C)C)nc2N2C[C@@H](C1)CC2(C)C]

(14S)-8-tert-Butyl-16-(2,2-dimethylpropyl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (8.6 mg, 0.01587 mmol) (30:70 mixture of diastereomers) was subjected to chiral SFC using a ChiralPak IG column (250×10 mm; 5 μm) at 35° C. Mobile phase was 18% MeOH (with 20 mM NH₃), 82% CO₂ at a 10 mL/min flow. Concentration of the sample was 24 mg/mL in methanol (no modifier), injection volume 70 μL with an outlet pressure of 152 bar, detection wavelength of 210 nm. Evaporation of the solvents gave two isomers as colorless films.

Peak 1: (14S)-8-tert-Butyl-16-(2,2-dimethylpropyl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (1.2 mg, 47%); ESI-MS m/z calc. 541.30865, found 542.48 (M+1)⁺; Retention time: 2.44 minutes (LC method A).

Peak 2: (14S)-8-tert-Butyl-16-(2,2-dimethylpropyl)-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (3.7 mg, 61%); ESI-MS m/z calc. 541.30865, found 542.52 (M+1)⁺; Retention time: 2.45 minutes (LC method A).

### Example 159: Preparation of (14S)-7-Benzyl-8-13-(2-{dispiro[2.0.24.131heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 427)

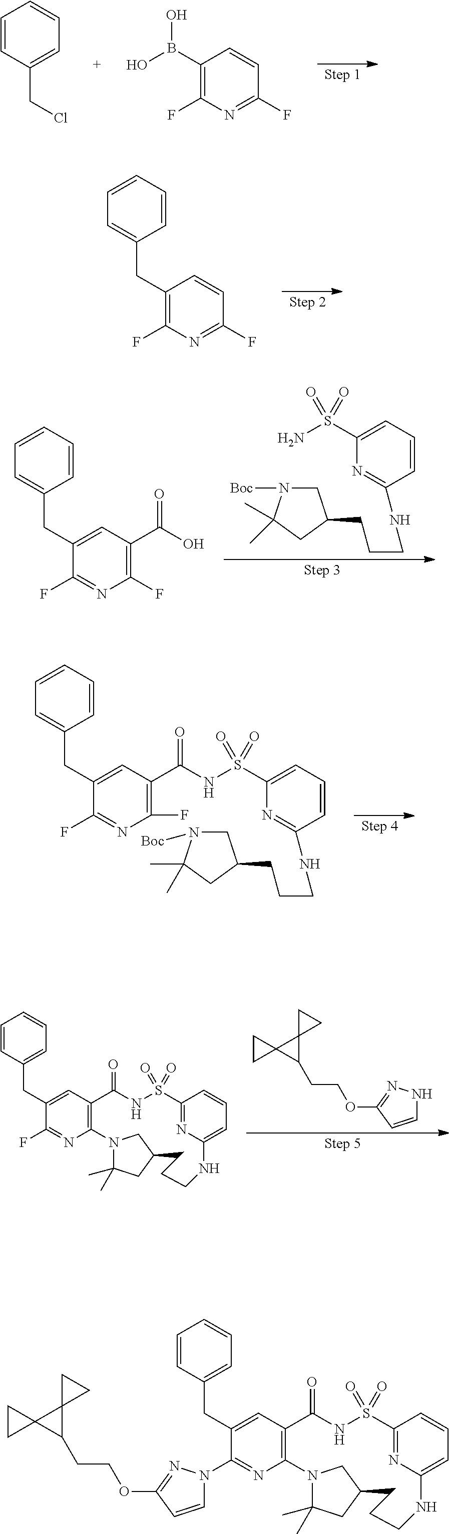

### Step 1: 3-Benzyl-2,6-difluoro-pyridine

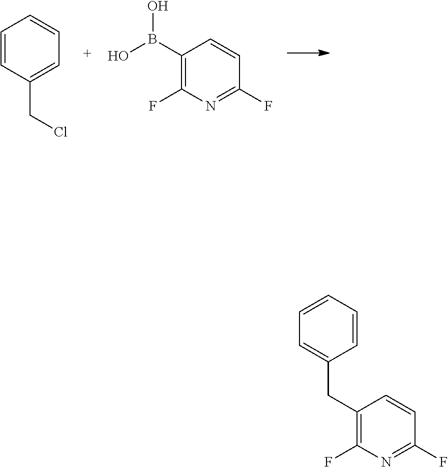 [SMILES: ClCc1ccccc1 ; OB(O)c1ccc(F)nc1F ; Fc1ccc(Cc2ccccc2)c(F)n1]

In a 250-mL round-bottomed flask, (2,6-difluoro-3-pyridyl)boronic acid (3.1351 g, 19.73 mmol), benzyl chloride (2.64 g, 20.86 mmol) and Pd(PPh₃)₄ (0.5525 g, 0.4781 mmol) were mixed with DME (80 mL) and aqueous Na₂CO₃ (40 mL of 2.0 M, 80.00 mmol). The resulting mixture was sparged with nitrogen gas for 5 min, and then stirred under reflux at 110° C. for 92 h. It was cooled to room temperature and filtered through Celite (rinsed with 100 mL EtOAc). The filtrate was diluted with H₂O (100 mL), and the layers were separated. The organic layer was kept aside, and the aqueous layer was extracted with EtOAc (100 mL). The combined organic extracts was washed with H₂O (100 mL) and brine (100 mL), then dried over Na₂SO₄, filtered, and evaporated in vacuo. Purification by silica gel chromatography (220 g of silica) using a gradient eluent of 0 to 15% EtOAc in hexanes gave a colorless oil, 3-benzyl-2,6-difluoro-pyridine (2.6395 g, 65%) ¹H NMR (400 MHz, DMSO-d₆) δ 8.06 (dt, J=9.9, 8.0 Hz, 1H), 7.33-7.27 (m, 2H), 7.26-7.20 (m, 3H), 7.14 (dd, J=8.0, 2.7 Hz, 1H), 3.98 (s, 2H).

### Step 2: 5-Benzyl-2,6-difluoro-pyridine-3-carboxylic acid

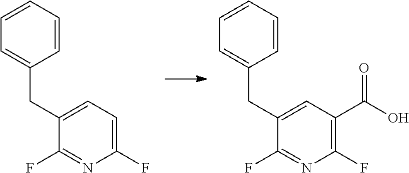 [SMILES: Fc1ccc(Cc2ccccc2)c(F)n1 ; O=C(O)c1cc(Cc2ccccc2)c(F)nc1F]

In a 100-mL round-bottomed flask, diisopropylamine (3.0 mL, 21.41 mmol) and THF (25 mL) were cooled to 78° C. under nitrogen atmosphere, upon which a hexanes solution of n-BuLi (8.0 mL of 2.5 M, 20.00 mmol) was added. The resulting mixture was stirred at 78° C. for 15 min, after which a THF (10 mL) solution of 3-benzyl-2,6-difluoro-pyridine (2.7675 g, 13.49 mmol) was added in one portion. The resulting mixture was stirred at 78° C. for 1 h, after which a stream of CO₂ (constant flow from a 16 gauge needle over 10 min) was bubbled into this mixture. The resulting mixture was stirred at 78° C. for 15 min, after which it was warmed to room temperature over 1 h. It was then poured into a flask containing 1 N HCl solution (100 mL), and the mixture was extracted with EtOAc (3×75 mL). The combined organic extracts was washed with H₂O (100 mL) and brine (100 mL), then dried over Na₂SO₄, filtered, and evaporated in vacuo to give a viscous yellow liquid that solidified under high vacuum overnight. The obtained product is probably only 50% pure: 5-benzyl-2,6-difluoro-pyridine-3-carboxylic acid (3.7 g, 55%).

### Step 3: tert-Butyl (4S)-4-[3-[[6-[(5-benzyl-2,6-difluoro-pyridine-3-carbonyl)sulfamoyl]-2-pyridyl]amino]propyl]-2,2-dimethyl-pyrrolidine-1-carboxylate

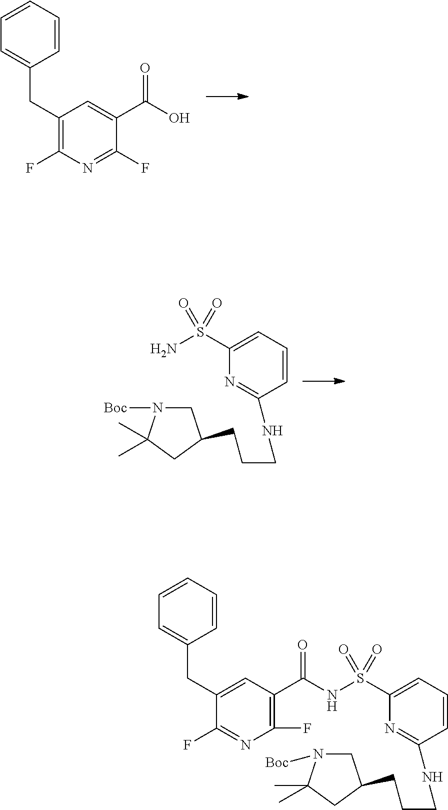 [SMILES: O=C(O)c1cc(Cc2ccccc2)c(F)nc1F ; CC(C)(C)OC(=O)N1C[C@@H](CCCNc2cccc(S(N)(=O)=O)n2)CC1(C)C ; CC(C)(C)OC(=O)N1C[C@@H](CCCNc2cccc(S(=O)(=O)NC(=O)c3cc(Cc4ccccc4)c(F)nc3F)n2)CC1(C)C]

In a 20-mL vial, 5-benzyl-2,6-difluoro-pyridine-3-carboxylic acid (402.5 mg, 0.8075 mmol) was mixed with DCM (7.0 mL) and cooled to 0° C., to which DMF (20 μL, 0.2583 mmol) and oxalyl chloride (200 μL, 2.293 mmol) were added. The resulting mixture was warmed to room temperature over 4 h. Upon verifying that the acyl chloride has indeed formed (with a small sample quench using morpholine to give (5-benzyl-2,6-difluoro-3-pyridyl)-morpholino-methanone), the mixture was evaporated in vacuo.

Separately, in a 20-mL microwave vial, a solution of tert-butyl (4S)-2,2-dimethyl-4-[3-[(6-sulfamoyl-2-pyridyl)amino]propyl]pyrrolidine-1-carboxylate (450 mg, 0.8574 mmol) and DIPEA (1.0 mL, 5.741 mmol) in DCM (6.0 mL) was prepared. The crude acyl chloride generated above was dissolved in DCM (3.0 mL) and added to the sulfonamide solution. The resulting mixture was stirred at room temperature for 72 h. It was then poured into 0.5 N HCl (20 mL), and extracted with EtOAc (3×10 mL). The combined organic extracts was washed with H₂O (20 mL) and brine (20 mL), then dried over Na₂SO₄, filtered, and evaporated in vacuo. Purification by silica gel chromatography (24 g of silica) using a gradient eluent of 0 to 80% EtOAc in hexanes gave a beige foam. This contained product, but was quite impure (˜30% pure by UPLC): tert-butyl (4S)-4-[3-[[6-[(5-benzyl-2,6-difluoro-pyridine-3-carbonyl)sulfamoyl]-2-pyridyl]amino]propyl]-2,2-dimethyl-pyrrolidine-1-carboxylate (245.6 mg, 14%) ESI-MS m/z calc. 643.264, found 644.3 (M+1)⁺; Retention time: 2.13 minutes (LC Method A).

### Step 4: (14S)-7-Benzyl-8-fluoro-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione

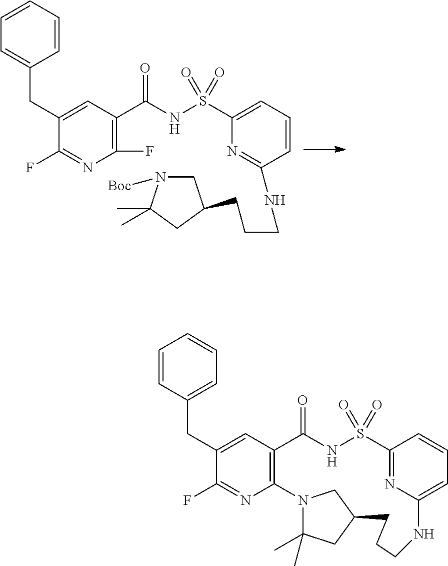 [SMILES: CC(C)(C)OC(=O)N1C[C@@H](CCCNc2cccc(S(=O)(=O)NC(=O)c3cc(Cc4ccccc4)c(F)nc3F)n2)CC1(C)C ; CC1(C)C[C@@H]2CCCNc3cccc(n3)S(=O)(=O)NC(=O)c3cc(Cc4ccccc4)c(F)nc3N1C2]

Stage 1: In a 20-mL vial, tert-butyl (4S)-4-[3-[[6-[(5-benzyl-2,6-difluoro-pyridine-3-carbonyl)sulfamoyl]-2-pyridyl]amino]propyl]-2,2-dimethyl-pyrrolidine-1-carboxylate (245.6 mg, 0.1145 mmol) was dissolved in DCM (2.0 mL), to which TFA (0.5 mL, 6.490 mmol) was added. The resulting solution was stirred at room temperature for 21 h. It was evaporated in vacuo, re-dissolved in MeOH (3.0 mL), then filtered and purified by reverse-phase preparative chromatography using a C₁₈ column and a gradient eluent of 1 to 60% MeCN in H₂O containing 5 mM HCl solution to give impure (˜40% pure) 5-benzyl-N-[[6-[3-[(3S)-5,5-dimethylpyrrolidin-3-yl]propylamino]-2-pyridyl]sulfonyl]-2,6-difluoro-pyridine-3-carboxamide (hydrochloride salt) (156.1 mg, 94%); ESI-MS m/z calc. 543.21155, found 544.2 (M+1)⁺; Retention time: 1.25 minutes (LC Method A).

Stage 2: In a 1-dram vial, the product from Stage 1 was dissolved in NMP (1.5 mL), to which K₂CO₃ (107.1 mg, 0.7749 mmol) was added. The resulting mixture was stirred at 160° C. for 21.5 h. It was cooled to room temperature, then was poured into 1 N HCl (3 mL). EtOAc (2 mL) was added. The phases were vigorously mixed and then allowed to settle into two layers. The organic layer was filtered and purified by reverse-phase preparative chromatography using a C₁₈ column and a gradient eluent of 1 to 99% acetonitrile in water containing 5 mM HCl solution to give a yellow solid, (14S)-7-benzyl-8-fluoro-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (19.7 mg, 33%); ¹H NMR (400 MHz, DMSO-d₆) δ 12.54 (s, 1H), 7.78 (d, J=10.0 Hz, 1H), 7.57 (dd, J=8.5, 7.2 Hz, 1H), 7.35-7.27 (m, 2H), 7.27-7.16 (m, 3H), 7.05 (d, J=7.1 Hz, 1H), 6.98 (d, J=9.1 Hz, 1H), 6.71 (d, J=8.5 Hz, 1H), 3.98-3.88 (m, 1H), 3.88-3.77 (m, 2H), 3.14-3.04 (m, 1H), 2.98-2.89 (m, 1H), 2.63 (t, J=10.4 Hz, 1H), 2.15-2.00 (m, 1H), 1.81 (dd, J=11.9, 5.3 Hz, 1H), 1.78-1.67 (m, 1H), 1.61-1.55 (m, 1H), 1.53 (s, 3H), 1.52-1.46 (m, 1H), 1.40 (s, 3H), 1.38-1.17 (m, 2H). ESI-MS m/z calc. 523.2053, found 524.1 (M+1)⁺; Retention time: 1.98 minutes (LC Method A).

### Step 5: (14S)-7-Benzyl-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (Compound 427)

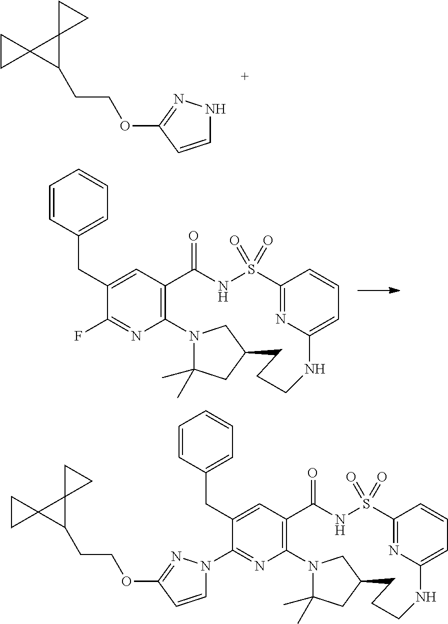

In a 1-dram vial, 3-(2-dispiro[2.0.24.13]heptan-7-ylethoxy)-1H-pyrazole (14.0 mg, 0.06854 mmol) and (14S)-7-benzyl-8-fluoro-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (14.0 mg, 0.02674 mmol) were mixed with K₂CO₃ (28.0 mg, 0.2026 mmol) and NMP (200 μL), and the resulting mixture was stirred at 170° C. for 17 h. After this time, it was cooled to room temperature, and a second portion of K₂CO₃ (28.0 mg, 0.2026 mmol) was added. The resulting mixture was stirred at 170° C. for an additional 26 h. It was cooled to room temperature, then 1 N HCl (1 mL) was added, followed by EtOAc (1 mL). The phases were vigorously mixed and then allowed to settle into two layers. The organic layer was filtered and purified by reverse-phase preparative chromatography using a C₁₈ column and a gradient eluent of 30 to 99% acetonitrile in water containing 5 mM HCl solution to give a beige powder, (14S)-7-benzyl-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(23),5(10),6,8,19,21-hexaene-2,2,4-trione (7.8 mg, 41%); ESI-MS m/z calc. 707.3254, found 708.3 (M+1)⁺; Retention time: 2.49 minutes; (LC Method A).

### Example 160: Preparation of 8-[3-(2-{Dispiro[2.0.24.13]heptan-7-yl}ethoxy)-1H-pyrazol-1-yl]-12,12-dimethyl-15-(2-phenylethyl)-2λ⁶-thia-3,9,11,18,23-pentaazatetracyclo[17.3.1.111,14.05,10]tetracosa-1(22),5(10),6,8,19(23),20-hexaene-2,2,4-trione (Compound 428)

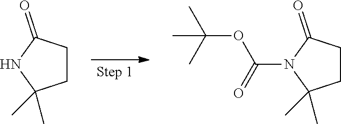

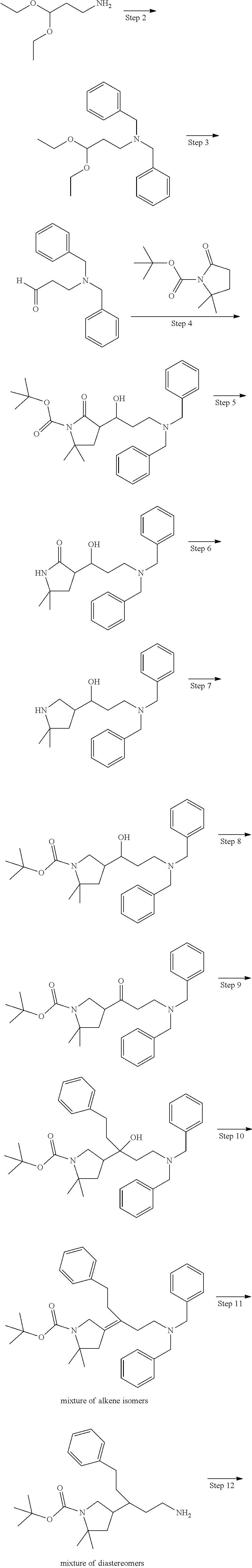

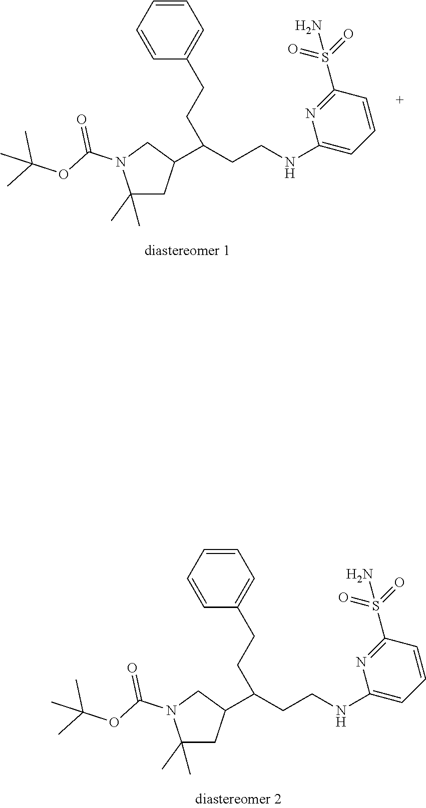 [SMILES: CC(C)(C)OC(=O)N1CC(C(CCNc2cccc(S(N)(=O)=O)n2)CCc2ccccc2)CC1(C)C ; CC(C)(C)OC(=O)N1CC(C(CCNc2cccc(S(N)(=O)=O)n2)CCc2ccccc2)CC1(C)C]

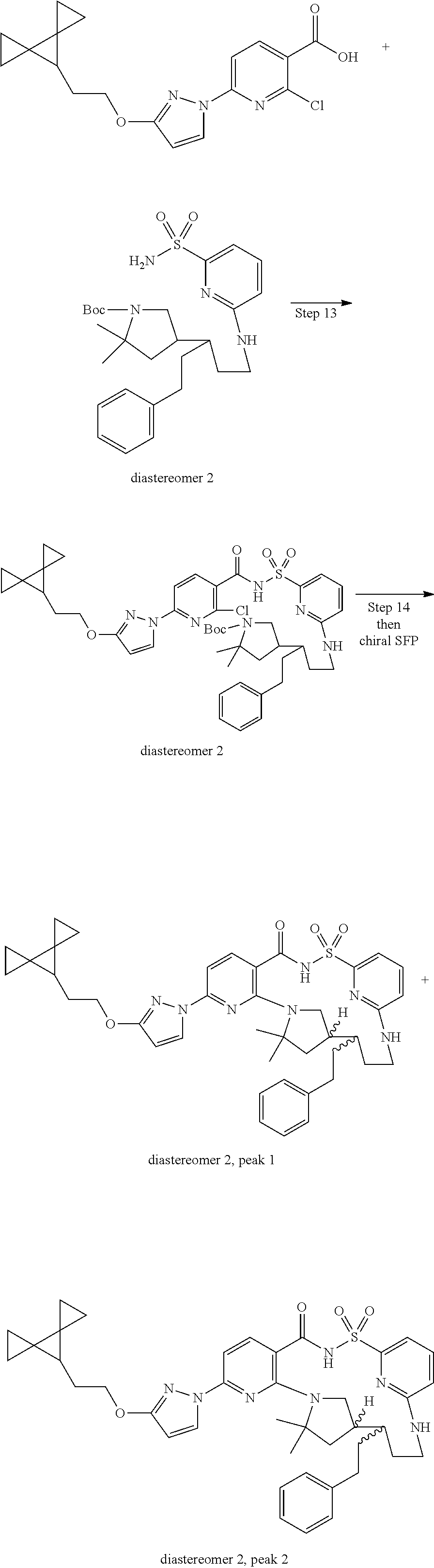 [SMILES: O=C(O)c1ccc(-n2ccc(OCCC3C4(CC4)C34CC4)n2)nc1Cl ; CC(C)(C)OC(=O)N1CC(C(CCNc2cccc(S(N)(=O)=O)n2)CCc2ccccc2)CC1(C)C ; CC(C)(C)OC(=O)N1CC(C(CCNc2cccc(S(=O)(=O)NC(=O)c3ccc(-n4ccc(OCCC5C6(CC6)C56CC6)n4)nc3Cl)n2)CCc2ccccc2)CC1(C)C ; CC1(C)CC2CN1c1nc(-n3ccc(OCCC4C5(CC5)C45CC5)n3)ccc1C(=O)NS(=O)(=O)c1cccc(n1)NCCC2CCc1ccccc1 ; CC1(C)CC2CN1c1nc(-n3ccc(OCCC4C5(CC5)C45CC5)n3)ccc1C(=O)NS(=O)(=O)c1cccc(n1)NCCC2CCc1ccccc1]

### Step 1: tert-Butyl 2,2-dimethyl-5-oxo-pyrrolidine-1-carboxylate

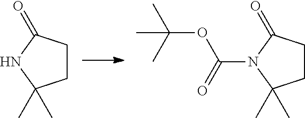

5,5-Dimethylpyrrolidin-2-one (10.6 g, 90.864 mmol) was dissolved in CH₃CN (110 mL) and the mixture was cooled in an ice-water bath. Boc₂O (21.6 g, 98.970 mmol) was added. The mixture was stirred at the same temperature for 15 min. DMAP (1.2 g, 9.8225 mmol) was then added. The mixture was warmed to RT and stirred for 15 h. It was then concentrated and the residue was taken into EtOAc (150 ml), washed with water (100 mL×2), brine, dried over MgSO₄, filtered and concentrated. The residue was purified by silica gel chromatography using 0-20% EtOAc in hexanes to afford the desired product as a white solid: tert-butyl 2,2-dimethyl-5-oxo-pyrrolidine-1-carboxylate (14 g, 69%); ESI-MS m/z calc. 213.13649, found 214.5 (M+1)⁺; Retention time: 2.86 minutes (LC Method B).

### Step 2: N,N-Dibenzyl-3,3-diethoxy-propan-1-amine

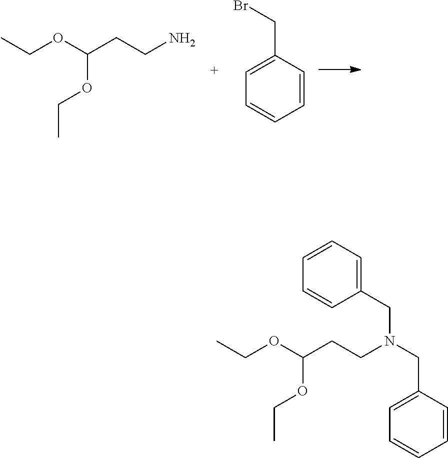

3,3-Diethoxypropan-1-amine (11.034 g, 12.5 mL, 72.703 mmol) was dissolved in EtOH (56 mL) and water (14 mL) at RT. Na₂CO₃ (22.75 g, 214.65 mmol) was added in one portion, followed by a solution of BnBr (41.995 g, 29.8 mL, 240.63 mmol) in EtOH (14 mL) which was added within 40 min. The mixture was then heated at 83° C. for 48 h. It was then cooled to RT and concentrated in vacuo. The residue was partitioned between DCM (200 mL) and water (200 mL). Layers were separated and the aqueous layer was extracted with more DCM (60 mL×3). The combined DCM solution was dried over MgSO₄, filtered and concentrated. The residue was taken into hexanes (300 mL) and stirred for 1 h. The supernatant was decanted and the residue (sticky yellow material) was rinsed twice with hexanes (50 mL×2). The combined hexanes solution was concentrated and the residue was purified by silica gel chromatography (120 g column), using 0-10% EtOAc in hexanes to afford the desired product as a colorless liquid, N,N-dibenzyl-3,3-diethoxy-propan-1-amine (18.56 g, 74%): ESI-MS m/z calc. 327.21982, found 328.6 (M+1)⁺; Retention time: 2.88 minutes (LC Method B).

### Step 3: 3-(Dibenzylamino)propanal

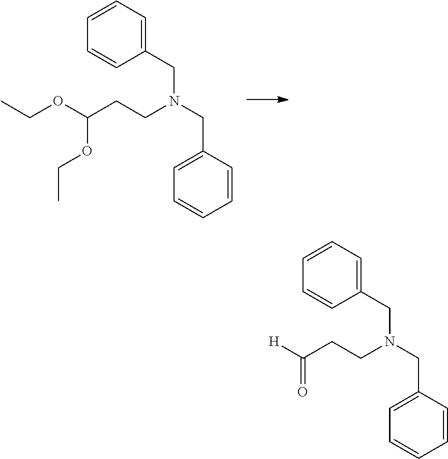 [SMILES: CCOC(CCN(Cc1ccccc1)Cc1ccccc1)OCC ; O=CCCN(Cc1ccccc1)Cc1ccccc1]

N,N-Dibenzyl-3,3-diethoxy-propan-1-amine (10 g, 29.011 mmol) was dissolved in THF (50 mL) at RT. Aqueous HCl (18 mL of 3 M, 54.000 mmol) was added. The mixture was stirred at RT for 4 h. It was then neutralized with NaHCO₃(sat. aq.) till pH 8. EtOAc was added (30 mL). Layers were separated and the EtOAc layer was washed with brine, dried over MgSO₄, filtered and concentrated. The residue (˜7.1 g) was taken into toluene (40 mL). The mixture was sonicated briefly and concentrated to about 5 mL volume. This solution was used directly in the next step. 3-(Dibenzylamino)propanal (7.1 g, 87%): ESI-MS m/z calc. 253.14667, found 272.5 (M+H2O+H=M+19)⁺; Retention time: 2.37 minutes (LC Method B).

### Step 4: tert-Butyl 4-[3-(dibenzylamino)-1-hydroxy-propyl]-2,2-dimethyl-5-oxo-pyrrolidine-1-carboxylate

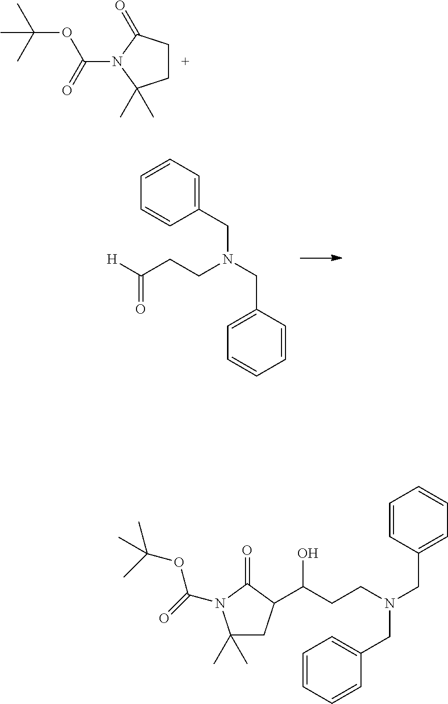

tert-Butyl 2,2-dimethyl-5-oxo-pyrrolidine-1-carboxylate (8 g, 35.635 mmol) was dissolved in THF (150 mL) and cooled under a nitrogen balloon to 78° C., stirring for 20 min. LDA in THF/heptane/ethylbenzene (19 mL of 2 M, 38.000 mmol) was added over 5 min. The mixture was stirred at 78° C. for 1 h. 3-(Dibenzylamino)propanal (7.1 g, 25.223 mmol) in toluene (10 mL) was added, followed by a rinse with THF (10 mL). The mixture was stirred at the same temperature for 30 min and then slowly warmed to 50° C. NH₄Cl (2 mL, sat. aq.) was added, followed by water (20 mL) and EtOAc (30 mL). Layers were separated. The organic layer was washed with brine, dried over MgSO₄, filtered and concentrated. The residue was purified by silica gel chromatography (40 g column) using 5-50% EtOAc in hexanes to afford the product as a colorless oil. tert-Butyl 4-[3-(dibenzylamino)-1-hydroxy-propyl]-2,2-dimethyl-5-oxo-pyrrolidine-1-carboxylate (12.5 g, 96%): ESI-MS m/z calc. 466.28317, found 467.5 (M+1)⁺; Retention time: 3.19 minutes (LC Method B).

### Step 5: 3-[3-(Dibenzylamino)-1-hydroxy-propyl]-5,5-dimethyl-pyrrolidin-2-one

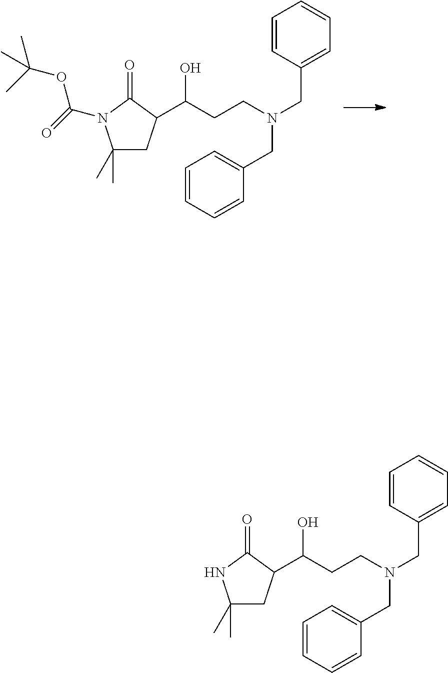 [SMILES: CC(C)(C)OC(=O)N1C(=O)C(C(O)CCN(Cc2ccccc2)Cc2ccccc2)CC1(C)C ; CC1(C)CC(C(O)CCN(Cc2ccccc2)Cc2ccccc2)C(=O)N1]

tert-Butyl 4-[3-(dibenzylamino)-1-hydroxy-propyl]-2,2-dimethyl-5-oxo-pyrrolidine-1-carboxylate (12.5 g, 24.110 mmol) was dissolved in DCM (50 mL) at RT. TFA (14.800 g, 10 mL, 129.80 mmol) was added. The mixture was stirred at RT for 2h. It was then concentrated to remove most volatiles. The residue was taken into DCM (100 mL) and treated with NaHCO₃(sat. aq.) till the aqueous phase pH turned basic. The layers were separated and the aqueous layer was extracted with more DCM (30 ml×2). The combined DCM solution was dried over MgSO₄, filtered and concentrated to afford the desired product as light brown oil. 3-[3-(Dibenzylamino)-1-hydroxy-propyl]-5,5-dimethyl-pyrrolidin-2-one (9 g, 97%): ESI-MS m/z calc. 366.23074, found 367.5 (M+1)⁺; Retention time: 2.58 minutes (LC Method B).

### Step 6: 3-(Dibenzylamino)-1-(5,5-dimethylpyrrolidin-3-yl)propan-1-ol

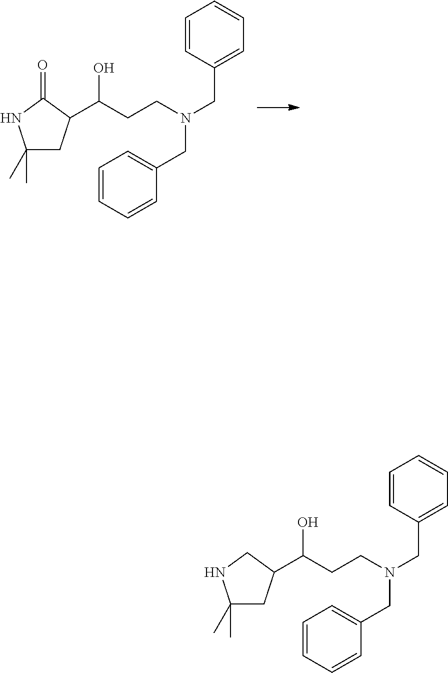 [SMILES: CC1(C)CC(C(O)CCN(Cc2ccccc2)Cc2ccccc2)C(=O)N1 ; CC1(C)CC(C(O)CCN(Cc2ccccc2)Cc2ccccc2)CN1]

Two batches of 3-[3-(dibenzylamino)-1-hydroxy-propyl]-5,5-dimethyl-pyrrolidin-2-one (9 g, 23.329 mmol and 250 mg, 0.6480 mmol) were combined and dissolved in THF (100 mL). After being cooled to 0° C., LAH (4.8 g, 5.2345 mL, 120.14 mmol) was added in small portions. The mixture was then stirred at RT for 30 min, before it was placed in an 80° C. oil bath, under a nitrogen balloon through a reflux condenser. The reaction was stirred at 80° C. for 24 h. It was then cooled to 0° C., diluted with ether (200 mL), and treated with sat. aq. solution of Rochelle salt (˜50 mL). Layers were separated. The organic layer was washed with brine, dried over MgSO₄, filtered and concentrated to afford crude product as a colorless oil. 3-(Dibenzylamino)-1-(5,5-dimethylpyrrolidin-3-yl)propan-1-ol (8.9 g, 97%): ESI-MS m/z calc. 352.25146, found 353.7 (M+1)⁺; Retention time: 2.21 minutes (LC Method B).

### Step 7: tert-Butyl 4-[3-(dibenzylamino)-1-hydroxy-propyl]-2,2-dimethyl-pyrrolidine-1-carboxylate

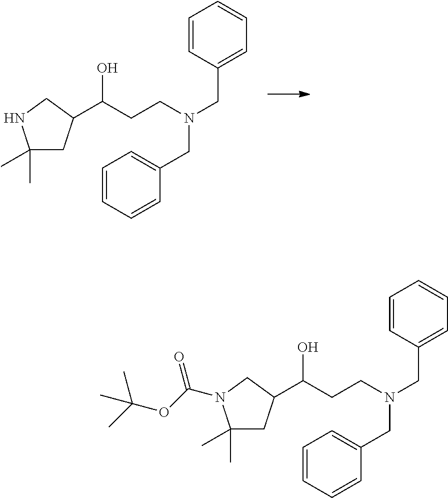 [SMILES: CC1(C)CC(C(O)CCN(Cc2ccccc2)Cc2ccccc2)CN1 ; CC(C)(C)OC(=O)N1CC(C(O)CCN(Cc2ccccc2)Cc2ccccc2)CC1(C)C]

3-(Dibenzylamino)-1-(5,5-dimethylpyrrolidin-3-yl)propan-1-ol (8.9 g, 22.723 mmol) was dissolved in THF (100 mL) at RT. Boc₂O (5 g, 5.2632 mL, 22.910 mmol) was added in one portion. The mixture was stirred at RT for 4 h. It was then concentrated and the residue was purified by silica gel chromatography (80 g column) using 0-30% EtOAc in hexanes to afford the product as a sticky colorless oil/white foam. tert-Butyl 4-[3-(dibenzylamino)-1-hydroxy-propyl]-2,2-dimethyl-pyrrolidine-1-carboxylate (8.98 g, 83%): ESI-MS m/z calc. 452.3039, found 453.6 (M+1)⁺; Retention time: 3.2 minutes (LC Method B).

### Step 8: tert-Butyl 4-[3-(dibenzylamino)propanoyl]-2,2-dimethyl-pyrrolidine-1-carboxylate

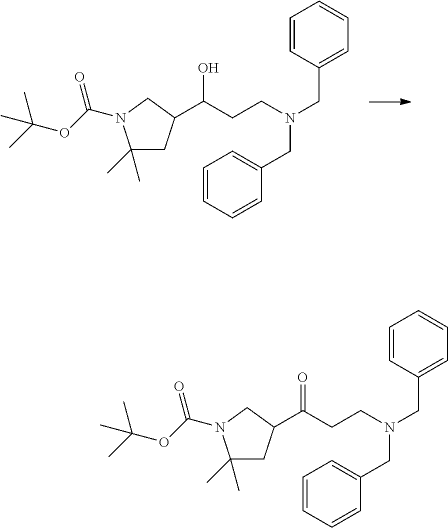 [SMILES: CC(C)(C)OC(=O)N1CC(C(O)CCN(Cc2ccccc2)Cc2ccccc2)CC1(C)C ; CC(C)(C)OC(=O)N1CC(C(=O)CCN(Cc2ccccc2)Cc2ccccc2)CC1(C)C]

tert-Butyl 4-[3-(dibenzylamino)-1-hydroxy-propyl]-2,2-dimethyl-pyrrolidine-1-carboxylate (4.4 g, 9.2349 mmol) was dissolved in DCM (50 mL) at RT. Dess-Martin periodinane (4.8 g, 10.751 mmol) was added in small portions. The reaction mixture was stirred under nitrogen for 3 h. More Dess-Martin periodinane (300 mg, 0.7073 mmol) was added. The mixture was stirred for another 1 h. Na₂S₂O₃ (5 g in 50 mL sat. aq. NaHCO₃) was added. The mixture was stirred for 30 min. Layers were separated. The DCM layer was dried over MgSO₄, filtered and concentrated. The residue was purified by silica gel chromatography (80 g column) using 5-40% EtOAc in hexanes to afford the product as a slightly yellowish oil. tert-Butyl 4-[3-(dibenzylamino)propanoyl]-2,2-dimethyl-pyrrolidine-1-carboxylate (2.78 g, 63%): ESI-MS m/z calc. 450.28824, found 451.8 (M+1)⁺; Retention time: 3.25 minutes (LC Method B).

### Step 9: tert-Butyl 4-[1-[2-(dibenzylamino)ethyl]-1-hydroxy-3-phenyl-propyl]-2,2-dimethyl-pyrrolidine-1-carboxylate

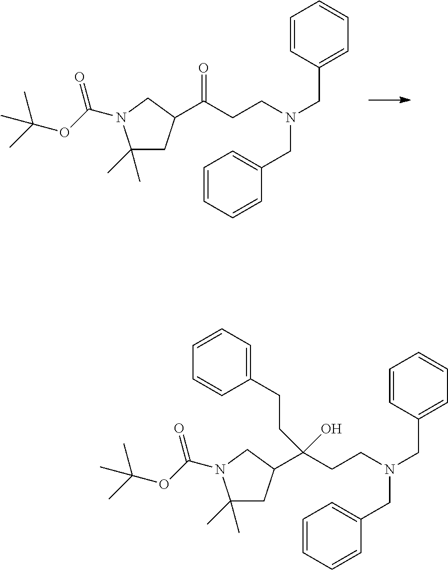 [SMILES: CC(C)(C)OC(=O)N1CC(C(=O)CCN(Cc2ccccc2)Cc2ccccc2)CC1(C)C ; CC(C)(C)OC(=O)N1CC(C(O)(CCc2ccccc2)CCN(Cc2ccccc2)Cc2ccccc2)CC1(C)C]

tert-Butyl 4-[3-(dibenzylamino)propanoyl]-2,2-dimethyl-pyrrolidine-1-carboxylate (2.78 g, 5.8609 mmol) was dissolved in THF (30 mL) and the solution was cooled to 78° C., stirring under nitrogen. A solution of chloro(2-phenylethyl)magnesium in THF (23 mL of 1 M, 23.000 mmol) was then added by syringe over 5 min. The mixture was stirred at the same temperature for 10 min, and then warmed by removing the cooling bath. The mixture was warmed to 0° C. over 90 min. NH₄Cl (10 ml, sat. aq.) was added, followed by EtOAc (50 mL). Layers were separated. The organic layer was washed with brine, dried over MgSO₄, filtered and concentrated. The residue was purified by silica gel chromatography (80 g column) using 0-30% EtOAc in hexanes to afford the product as a pale yellowish oil. tert-Butyl 4-[1-[2-(dibenzylamino)ethyl]-1-hydroxy-3-phenyl-propyl]-2,2-dimethyl-pyrrolidine-1-carboxylate (2.43 g, 71%): ESI-MS m/z calc. 556.3665, found 557.7 (M+1)⁺; Retention time: 3.65 minutes (LC Method B).

### Step 10: tert-Butyl 4-[1-[2-(dibenzylamino)ethyl]-3-phenyl-propylidene]-2,2-dimethyl-pyrrolidine-1-carboxylate

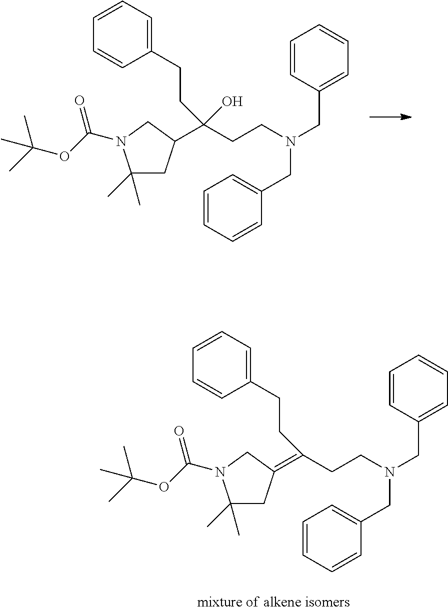 [SMILES: CC(C)(C)OC(=O)N1CC(C(O)(CCc2ccccc2)CCN(Cc2ccccc2)Cc2ccccc2)CC1(C)C ; CC(C)(C)OC(=O)N1C/C(=C(\CCc2ccccc2)CCN(Cc2ccccc2)Cc2ccccc2)CC1(C)C]

tert-Butyl 4-[1-[2-(dibenzylamino)ethyl]-1-hydroxy-3-phenyl-propyl]-2,2-dimethyl-pyrrolidine-1-carboxylate (2.43 g, 4.1462 mmol) was dissolved in pyridine (25 mL). The mixture was cooled to 0° C. and stirred for 15 min. SOCl₂ (4.8930 g, 3 mL, 41.128 mmol) was added dropwise. The mixture was stirred for 2 h at the same temperature. EtOAc (80 mL) was added, followed by water (30 mL) and NaHCO₃(30 mL, sat. aq.). Layers were separated. The EtOAc layer was washed with more NaHCO₃(30 mL×3, sat. aq.). It was then concentrated. The residue was purified by silica gel chromatography (80 g column) using 0 to 30% EtOAc in hexanes to afford the product (pale yellow oil). tert-Butyl 4-[1-[2-(dibenzylamino)ethyl]-3-phenyl-propylidene]-2,2-dimethyl-pyrrolidine-1-carboxylate as alkene isomers (1.1 g, 47%): ESI-MS m/z calc. 538.35596, found 539.7 (M+1)⁺; Retention time: 3.97 minutes (LC Method B).

### Step 11: tert-Butyl 4-[1-(2-aminoethyl)-3-phenyl-propyl]-2,2-dimethyl-pyrrolidine-1-carboxylate

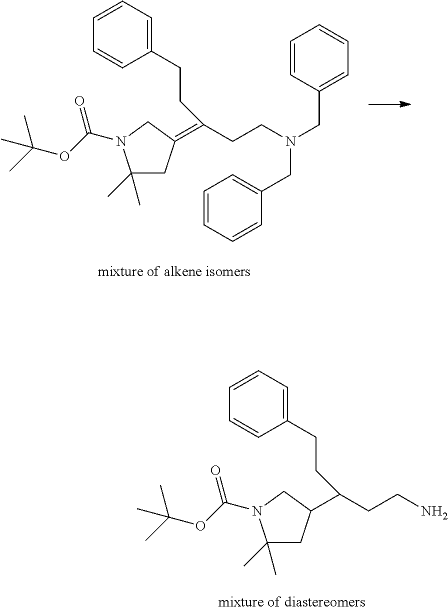 [SMILES: CC(C)(C)OC(=O)N1C/C(=C(\CCc2ccccc2)CCN(Cc2ccccc2)Cc2ccccc2)CC1(C)C ; CC(C)(C)OC(=O)N1CC(C(CCN)CCc2ccccc2)CC1(C)C]

tert-Butyl 4-[1-[2-(dibenzylamino)ethyl]-3-phenyl-propylidene]-2,2-dimethyl-pyrrolidine-1-carboxylate as a mixture of alkene isomers (1.1 g, 1.9396 mmol) was dissolved in MeOH (12 mL). Pd(OH)₂ on carbon (500 mg, 0.3560 mmol) was added. The mixture was vacuumed and purged with H₂ three times in a hydrogenation flask. It was then hydrogenated on a Parr shaker at 60 psi H₂ pressure for 36 h. The mixture was filtered through a Celite pad, then rinsed with MeOH (20 mL×3). The combined MeOH solutions were concentrated to afford the product. tert-Butyl 4-[1-(2-aminoethyl)-3-phenyl-propyl]-2,2-dimethyl-pyrrolidine-1-carboxylate, as a mixture of diastereomers: ESI-MS m/z calc. 360.27768, found 361.6 (M+1)⁺; Retention time: 3.19 minutes and 3.34 minutes (LC Method B).

### Step 12: tert-Butyl 2,2-dimethyl-4-[1-(2-phenylethyl)-3-[(6-sulfamoyl-2-pyridyl)amino]propyl]pyrrolidine-1-carboxylate; tert-butyl 2,2-dimethyl-4-[1-(2-phenylethyl)-3-[(6-sulfamoyl-2-pyridyl)amino]propyl]pyrrolidine-1-carboxylate

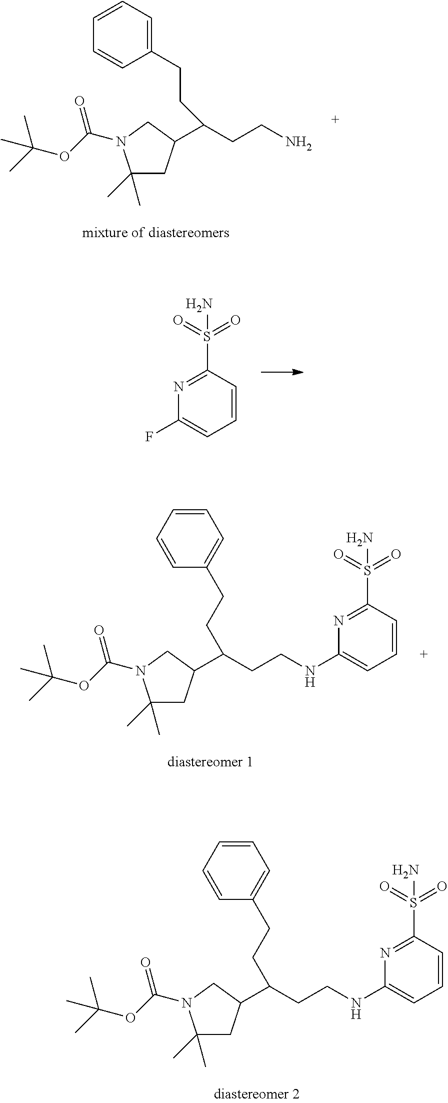 [SMILES: CC(C)(C)OC(=O)N1CC(C(CCN)CCc2ccccc2)CC1(C)C ; NS(=O)(=O)c1cccc(F)n1 ; CC(C)(C)OC(=O)N1CC(C(CCNc2cccc(S(N)(=O)=O)n2)CCc2ccccc2)CC1(C)C ; CC(C)(C)OC(=O)N1CC(C(CCNc2cccc(S(N)(=O)=O)n2)CCc2ccccc2)CC1(C)C]

tert-Butyl 4-[1-(2-aminoethyl)-3-phenyl-propyl]-2,2-dimethyl-pyrrolidine-1-carboxylate as a mixture of diastereomers (750 mg, 1.8722 mmol) was dissolved in DMSO (3 mL). 6-Fluoropyridine-2-sulfonamide (495 mg, 2.8098 mmol) was added, followed by DIPEA (964.60 mg, 1.3 mL, 7.4635 mmol). The mixture was heated at 115° C. under nitrogen for 24 h. It was then cooled to RT and diluted with water (20 mL) and EtOAc (30 mL). Layers were separated and the organic layer was washed with water (15 mL×2) and concentrated. The residue was purified by HPLC using 50 to 100% acetonitrile in 5 mM HCl in water to afford two separated isomers.

Diastereomer 1, tert-butyl 2,2-dimethyl-4-[1-(2-phenylethyl)-3-[(6-sulfamoyl-2-pyridyl)amino]propyl]pyrrolidine-1-carboxylate (179.6 mg, 18%); ¹H NMR (500 MHz, DMSO-d₆) δ 7.52 (t, J=7.8, 7.8 Hz, 1H), 7.31-7.10 (m, 5H), 7.02 (s, 2H), 6.99-6.84 (m, 2H), 6.60 (d, J=8.5 Hz, 1H), 3.67-3.44 (m, 1H), 3.34-3.26 (m, 2H), 2.82 (dd, J=12.2, 9.5 Hz, 1H), 2.71-2.58 (m, 1H), 2.58-2.51 (m, 1H), 2.24-2.07 (m, 1H), 2.00 1.80 (m, 1H), 1.72-1.30 (m, 18H), 1.23 (s, 3H). ESI-MS m/z calc. 516.27704, found 517.4 (M+1)⁺; Retention time: 3.0 minutes (LC Method H).

Diastereomer 2, tert-butyl 2,2-dimethyl-4-[1-(2-phenylethyl)-3-[(6-sulfamoyl-2-pyridyl)amino]propyl]pyrrolidine-1-carboxylate (290.2 mg, 29%); ¹H NMR (500 MHz, DMSO-d₆) δ 7.52 (dd, J=8.4, 7.2 Hz, 1H), 7.29-7.10 (m, 5H), 7.03 (s, 2H), 6.97 (d, J=7.2 Hz, 1H), 6.93 (t, J=5.4, 5.4 Hz, 1H), 6.61 (d, J=8.4 Hz, 1H), 3.66-3.45 (m, 1H), 3.39-3.31 (m, 2H), 2.87 (t, J=10.7, 10.7 Hz, 1H), 2.70-2.51 (m, 2H), 2.29-2.08 (m, 1H), 1.98-1.76 (m, 1H), 1.72-1.27 (m, 18H), 1.23 (s, 3H). ESI-MS m/z calc. 516.27704, found 517.4 (M+1)⁺; Retention time: 3.15 minutes (LC Method H).

### Step 13: tert-Butyl 4-[1-({6-[({2-chloro-6-[3-(2-{dispiro[2.0.24.13]heptan-7-yl}ethoxy)-1H-pyrazol-1-yl]pyridin-3-yl}formamido)sulfonyl]pyridin-2-yl}amino)-5-phenylpentan-3-yl]-2,2-dimethylpyrrolidine-1-carboxylate

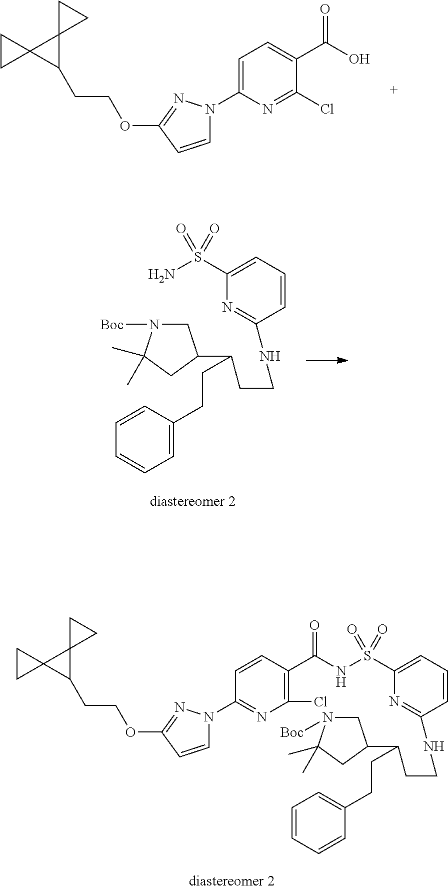 [SMILES: O=C(O)c1ccc(-n2ccc(OCCC3C4(CC4)C34CC4)n2)nc1Cl ; CC(C)(C)OC(=O)N1CC(C(CCNc2cccc(S(N)(=O)=O)n2)CCc2ccccc2)CC1(C)C ; CC(C)(C)OC(=O)N1CC(C(CCNc2cccc(S(=O)(=O)NC(=O)c3ccc(-n4ccc(OCCC5C6(CC6)C56CC6)n4)nc3Cl)n2)CCc2ccccc2)CC1(C)C]

In a 20-mL vial, 2-chloro-6-[3-(2-dispiro[2.0.24.13]heptan-7-ylethoxy)pyrazol-1-yl]pyridine-3-carboxylic acid (230.1 mg, 0.5756 mmol) was dissolved in THF (5.0 mL), to which CDI (150 mg, 0.9251 mmol) was added. The resulting mixture was stirred at room temperature for 15 h. After this time, diastereomer 2 of tert-butyl 2,2-dimethyl-4-[1-(2-phenylethyl)-3-[(6-sulfamoyl-2-pyridyl)amino]propyl]pyrrolidine-1-carboxylate (290.2 mg, 0.5397 mmol) and DBU (500 4, 3.343 mmol) were added, and the resulting mixture was stirred at room temperature for 24 h. After this time, 1 N HCl (5 mL) was added, and the mixture was extracted with EtOAc (3×3 mL). The combined organic extracts was washed with H₂O (5 mL) and brine (5 mL), then dried over Na₂SO₄, filtered, and evaporated in vacuo to give 700 mg of a brown solid. Purification by silica gel chromatography (24 g of silica) using a gradient eluent of 0 to 70% EtOAc in hexanes gave tert-butyl 4-[1-({6-[({2-chloro-6-[3-(2-{dispiro[2.0.24.13]heptan-7-yl}ethoxy)-1H-pyrazol-1-yl]pyridin-3-yl}formamido)sulfonyl]pyridin-2-yl}amino)-5-phenylpentan-3-yl]-2,2-dimethylpyrrolidine-1-carboxylate (294.6 mg, 60%); ESI-MS m/z calc. 857.3701, found 858.7 (M+1)⁺; Retention time: 1.86 minutes (LC method G).

### Step 14: 8-13-(2-{Dispiro[2.0.24.131heptan-7-yl}ethoxy)-1H-pyrazol-1-yl]-12,12-dimethyl-15-(2-phenylethyl)-2λ⁶-thia-3,9,11,18,23-pentaazatetracyclo [17.3.1.111,14.05,10]tetracosa-1(22),5(10),6,8,19(23),20-hexaene-2,2,4-trione (Compound 428a) and 8-[3-(2-{dispiro[2.0.24.13]heptan-7-yl}ethoxy)-1H-pyrazol-1-yl]-12,12-dimethyl-15-(2-phenylethyl)-2λ⁶-thia-3,9,11,18,23-pentaazatetracyclo[17.3.1.111,14.05,10]tetracosa-1(22),5(10),6,8,19(23),20-hexaene-2,2,4-trione (Compound 428b)

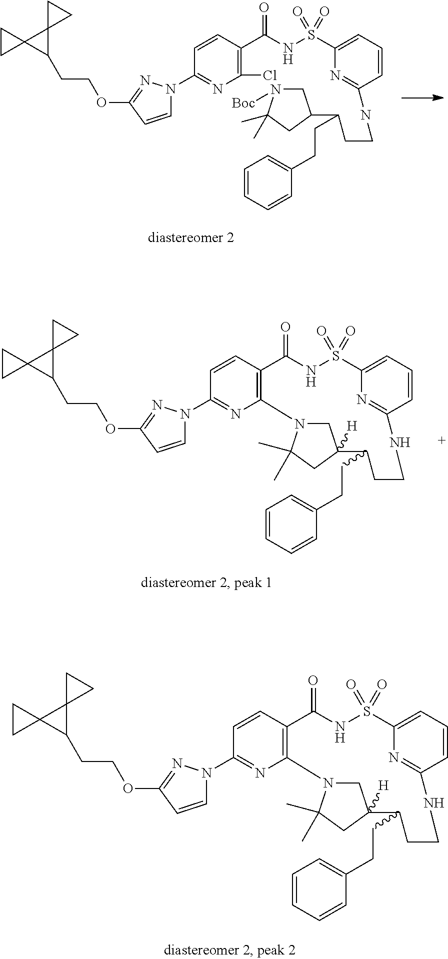 [SMILES: CC(C)(C)OC(=O)N1CC(C(CCNc2cccc(S(=O)(=O)NC(=O)c3ccc(-n4ccc(OCCC5C6(CC6)C56CC6)n4)nc3Cl)n2)CCc2ccccc2)CC1(C)C ; CC1(C)CC2CN1c1nc(-n3ccc(OCCC4C5(CC5)C45CC5)n3)ccc1C(=O)NS(=O)(=O)c1cccc(n1)NCCC2CCc1ccccc1 ; CC1(C)CC2CN1c1nc(-n3ccc(OCCC4C5(CC5)C45CC5)n3)ccc1C(=O)NS(=O)(=O)c1cccc(n1)NCCC2CCc1ccccc1]

Stage 1: In a 20-mL vial, tert-butyl 4-[1-({6-[({2-chloro-6-[3-(2-{dispiro[2.0.24.13]heptan-7-yl}ethoxy)-1H-pyrazol-1-yl]pyridin-3-yl}formamido)sulfonyl]pyridin-2-yl}amino)-5-phenylpentan-3-yl]-2,2-dimethylpyrrolidine-1-carboxylate (294.6 mg, 0.3226 mmol) was dissolved in DCM (4.0 mL). TFA (1.0 mL, 12.98 mmol) was added, and the resulting solution was allowed to stand at room temperature for 16 h. The mixture was then evaporated in vacuo, mixed with dry dioxane (3 mL), and evaporated to dryness to give 500 mg (>100%) of an orange oil, 2-chloro-N-[(6-{[3-(5,5-dimethylpyrrolidin-3-yl)-5-phenylpentyl]amino}pyridin-2-yl)sulfonyl]-6-[3-(2-{dispiro[2.0.24.13]heptan-7-yl}ethoxy)-1H-pyrazol-1-yl]pyridine-3-carboxamide (trifluoroacetate salt).

Stage 2: In a 20-mL microwave vial, the product from Stage 1 was dissolved in NMP (5.0 mL) and mixed with K₂CO₃ (352.1 mg, 2.548 mmol). The resulting mixture was capped with a microwave cap and stirred at 150° C. for 20 h. The reaction mixture was then cooled to room temperature, poured onto 1 N HCl (10 mL), and extracted with EtOAc (3×10 mL). The combined organic extracts was washed with H₂O (10 mL) and brine (10 mL), then dried over Na₂SO₄, filtered, and evaporated in vacuo. Purification by silica gel chromatography (24 g of silica) using a gradient eluent of 0 to 60% EtOAc in hexanes gave a white solid (143.1 mg, 61% yield). This product was dissolved in MeOH (ca. 4.5 mL) to achieve a concentration of ca. 32 mg/mL. Separation of the enantiomers was achieved with an SFC purification method using a ChiralPak IG column (250×21.2 mm, 5 μm particle size), with a mobile phase of 46% MeOH (+20 mM NH₃)+54% CO₂, a flow rate of 70 mL/min, an injection volume of 500 μL, and a pressure of 177 bar. The collected batches were labeled “Diastereomer 2 Peak 1” (60.3 mg, 25%) and “Diastereomer 2 Peak 2” (58.9 mg, 25%).

“Diastereomer 2 Peak 1”: 8-[3-(2-{dispiro[2.0.24.13]heptan-7-yl}ethoxy)-1H-pyrazol-1-yl]-12,12-dimethyl-15-(2-phenylethyl)-2λ⁶-thia-3,9,11,18,23-pentaazatetracyclo[17.3.1.111,14.05,10]tetracosa-1 (22),5(10),6,8,19(23),20-hexaene-2,2,4-tri one (ammonium salt) (60.3 mg, 25%) ESI-MS m/z calc. 721.341, found 722.3 (M+1)⁺; Retention time: 2.57 minutes; (LC Method A).

“Diastereomer 2 Peak 2”: 8-[3-(2-{dispiro[2.0.24.13]heptan-7-yl}ethoxy)-1H-pyrazol-1-yl]-12,12-dimethyl-15-(2-phenylethyl)-2λ⁶-thia-3,9,11,18,23-pentaazatetracyclo[17.3.1.111,14.05,10]tetracosa-1(22),5(10),6,8,19(23),20-hexaene-2,2,4-trione (ammonium salt) (58.9 mg, 25%)¹H NMR (400 MHz, DMSO-d₆) δ 12.73-12.24 (bs, 1H), 8.19 (d, J=2.7 Hz, 1H), 7.86-7.66 (m, 1H), 7.64-7.49 (m, 1H), 7.34-7.22 (m, 4H), 7.21-7.13 (m, 1H), 7.15-6.99 (m, 2H), 6.94-6.82 (m, 1H), 6.77-6.61 (m, 1H), 6.06 (s, 1H), 4.27-4.15 (m, 2H), 3.84-3.61 (m, 1H), 3.16-3.02 (m, 1H), 2.98-2.75 (m, 1H), 2.73-2.52 (m, 2H), 2.22-2.09 (m, 1H), 2.05-1.88 (m, 2H), 1.81 (q, J 6.7 Hz, 2H), 1.76-1.62 (m, 2H), 1.57 (s, 3H), 1.53 (s, 3H), 1.50-1.42 (m, 2H), 1.43-1.33 (m, 1H), 0.89-0.77 (m, 4H), 0.69-0.61 (m, 2H), 0.54-0.45 (m, 2H). [Note: 2H is missing from the overall count of 47 from the product (C₄₀H₄₇N₇O₄S).] ESI-MS m/z calc. 721.341, found 722.3 (M+1)⁺; Retention time: 2.57 minutes (LC Method A).

### Example 161: Preparation of (14S)-8-[3-(2-{Dispiro[2.0.24.13]heptan-7-yl}ethoxy)-1H-pyrazol-1-yl]-12,12-dimethyl-2λ⁶-thia-3,7,9,11,18,23-hexaazatetracyclo [17.3.1.111,14.05,10]tetracosa-1(22),5,7,9,19(23),20-hexaene-2,2,4-trione (Compound 429)

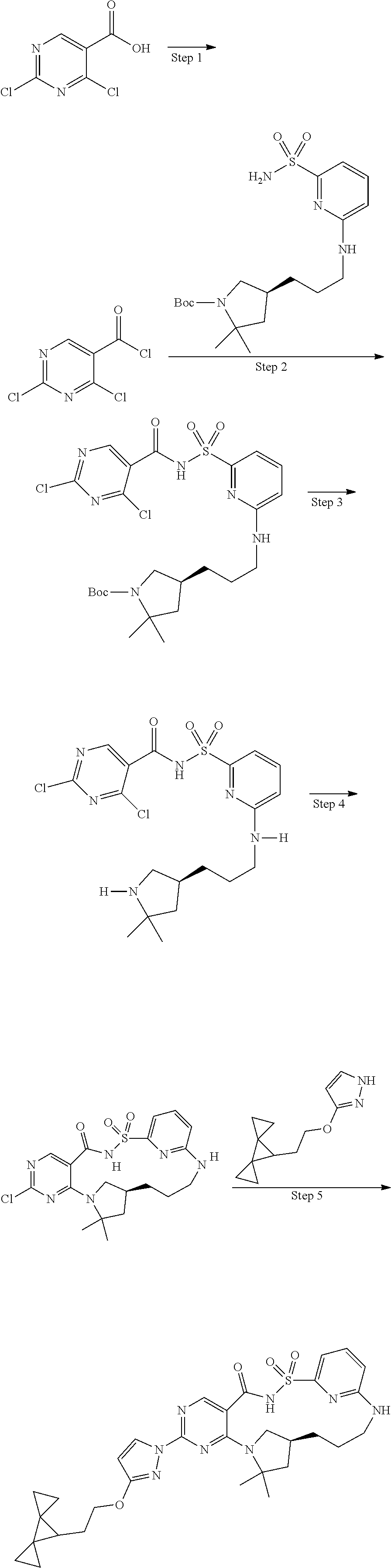

### Step 1: 2,4-Dichloropyrimidine-5-carbonyl chloride

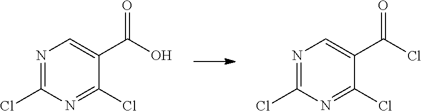 [SMILES: O=C(O)c1cnc(Cl)nc1Cl ; O=C(Cl)c1cnc(Cl)nc1Cl]

To 2,4-dichloropyrimidine-5-carboxylic acid (1 g, 5.1817 mmol) was added thionyl chloride (13.120 g, 8 mL, 110.28 mmol) and the resulting suspension was refluxed overnight. The reaction mixture was concentrated under reduced pressure to provide 2,4-dichloropyrimidine-5-carbonyl chloride (1.31 g, 114%)¹H NMR (400 MHz, CDCl₃) δ 9.26 (s, 1H).

### Step 2: tert-Butyl (4S)-4-[3-[[6-[(2,4-dichloropyrimidine-5-carbonyl)sulfamoyl]-2-pyridyl]amino]propyl]-2,2-dimethyl-pyrrolidine-1-carboxylate

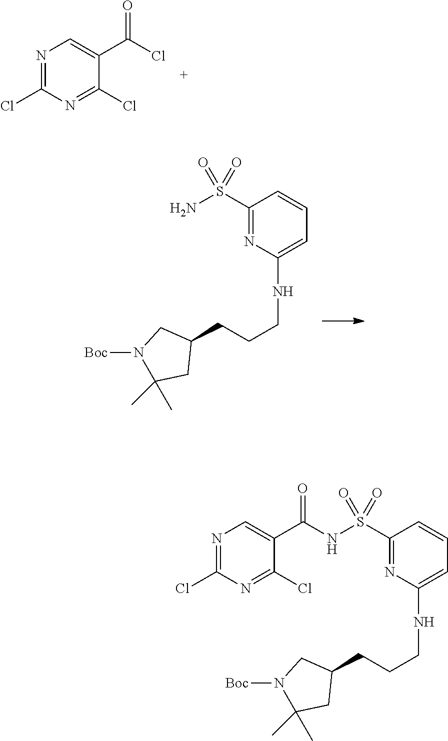 [SMILES: O=C(Cl)c1cnc(Cl)nc1Cl ; CC(C)(C)OC(=O)N1C[C@@H](CCCNc2cccc(S(N)(=O)=O)n2)CC1(C)C ; CC(C)(C)OC(=O)N1C[C@@H](CCCNc2cccc(S(=O)(=O)NC(=O)c3cnc(Cl)nc3Cl)n2)CC1(C)C]

tert-Butyl (4S)-2,2-dimethyl-4-[3-[(6-sulfamoyl-2-pyridyl)amino]propyl]pyrrolidine-1-carboxylate (176 mg, 0.4266 mmol) in DCM (12 mL) was added to 2,4-dichloropyrimidine-5-carbonyl chloride (100 mg, 0.4730 mmol) in DCM (12 mL) dropwise then TEA (50.820 mg, 0.07 mL, 0.5022 mmol) was added and the reaction was stirred 4 h at room temperature. More TEA (50.820 mg, 0.07 mL, 0.5022 mmol) was added and the reaction mixture stirred another 3 h. The reaction mixture was concentrated then purified on silica gel using 0 to 100% ethyl acetate in heptane, then with 0 to 30% methanol in DCM, to provide tert-butyl (4S)-4-[3-[[6-[(2,4-dichloropyrimidine-5-carbonyl)sulfamoyl]-2-pyridyl]amino]propyl]-2,2-dimethyl-pyrrolidine-1-carboxylate (150 mg, 54%): ¹H NMR (400 MHz, DMSO-d₆) δ 8.98 (s, 1H), 7.62 (t, J=7.8 Hz, 1H), 7.23 (br. s., 1H), 7.17 (d, J=7.1 Hz, 1H), 6.74 (d, J=8.3 Hz, 1H), 3.54-3.51 (m, 1H), 3.24-3.19 (m, 2H), 2.81-2.74 (m, 1H), 2.13-2.01 (m, 1H), 1.93-1.75 (m, 1H), 1.58-1.43 (m, 2H), 1.41-1.28 (m, 15H), 1.22 (s, 3H). ESI-MS m/z calc. 586.1532, found 487.0 (M-Boc+2H=M−99)⁺; Retention time: 2.29 minutes (LC method E).

### Step 3: 2,4-Dichloro-N-[[6-[3-[(35)-5,5-dimethylpyrrolidin-3-yl]propylamino]-2-pyridyl]sulfonyl]pyrimidine-5-carboxamide (hydrochloride salt)

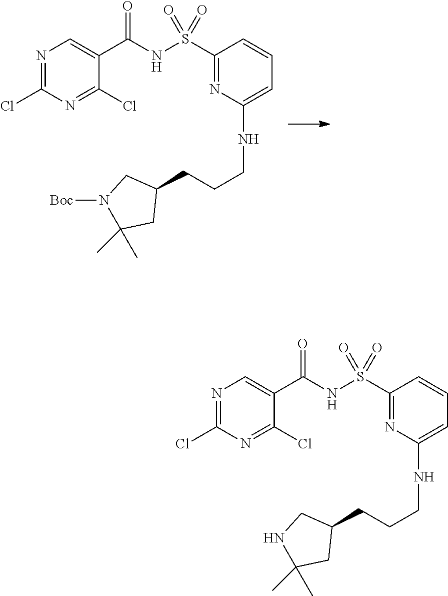 [SMILES: CC(C)(C)OC(=O)N1C[C@@H](CCCNc2cccc(S(=O)(=O)NC(=O)c3cnc(Cl)nc3Cl)n2)CC1(C)C ; CC1(C)C[C@H](CCCNc2cccc(S(=O)(=O)NC(=O)c3cnc(Cl)nc3Cl)n2)CN1]

Hydrochloric acid (4 M in dioxane) (7.25 mL of 4 M, 29.000 mmol) was added dropwise under nitrogen at room temperature to a solution of tert-butyl (4S)-4-[3-[[6-[(2,4-dichloropyrimidine-5-carbonyl)sulfamoyl]-2-pyridyl]amino]propyl]-2,2-dimethyl-pyrrolidine-1-carboxylate (145 mg, 0.2468 mmol) in DCM (7.25 mL). After stirring for 15 min, concentration under reduced pressure provided 2,4-dichloro-N-[[6-[3-[(3S)-5,5-dimethylpyrrolidin-3-yl]propylamino]-2-pyridyl]sulfonyl]pyrimidine-5-carboxamide (hydrochloride salt) (144 mg, 104%) ESI-MS m/z calc. 486.1008, found 487.0 (M+1)⁺; Retention time: 1.32 minutes (LC method E).

### Step 4: (14S)-8-chloro-12,12-dimethyl-2λ⁶-thia-3,7,9,11,18,23-hexaazatetracyclo[17.3.1.111,14.05,10]tetracosa-1(22),5,7,9,19(23),20-hexaene-2,2,4-trione

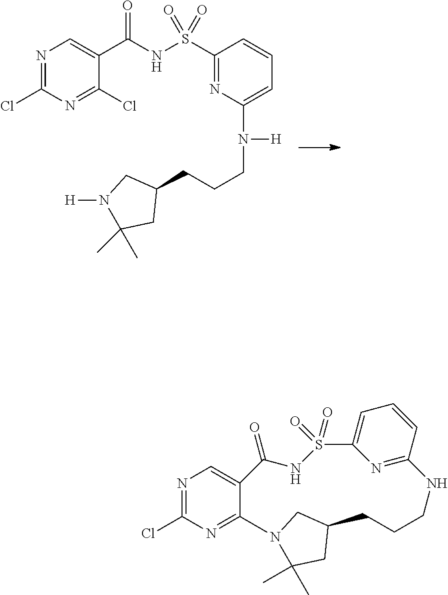 [SMILES: CC1(C)C[C@H](CCCNc2cccc(S(=O)(=O)NC(=O)c3cnc(Cl)nc3Cl)n2)CN1 ; CC1(C)C[C@@H]2CCCNc3cccc(n3)S(=O)(=O)NC(=O)c3cnc(Cl)nc3N1C2]

Under nitrogen, in a microwave vial, DIPEA (20 mg, 0.0270 mL, 0.1547 mmol) was added to 2,4-dichloro-N-[[6-[3-[(3S)-5,5-dimethylpyrrolidin-3-yl]propylamino]-2-pyridyl]sulfonyl]pyrimidine-5-carboxamide (20 mg, 0.0410 mmol), and then NMP (0.2 mL) was added. The vial was capped and the reaction mixture was heated to 125° C. for 1 h. The LCMS showed conversion to the desired product, (14S)-8-chloro-12,12-dimethyl-2λ⁶-thia-3,7,9,11,18,23-hexaazatetracyclo[17.3.1.111,14.05,10]tetracosa-1(22),5,7,9,19(23),20-hexaene-2,2,4-trione. ESI-MS m/z calc. 450.12408, found 451.2 (M+1)⁺; Retention time: 1.78 minutes (LC method E).

### Step 5: (14S)-8-13-(2-{Dispiro[2.0.24.131heptan-7-yl}ethoxy)-1H-pyrazol-1-yl]-12,12-dimethyl-2λ⁶-thia-3,7,9,11,18,23-hexaazatetracyclo[17.3.1.111,14.05,10]tetracosa-1(22),5,7,9,19(23),20-hexaene-2,2,4-trione (Compound 429)

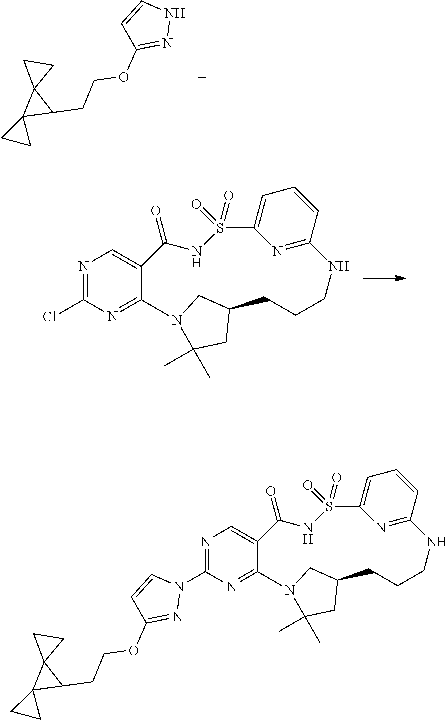 [SMILES: c1cc(OCCC2C3(CC3)C23CC3)n[nH]1 ; CC1(C)C[C@@H]2CCCNc3cccc(n3)S(=O)(=O)NC(=O)c3cnc(Cl)nc3N1C2 ; CC1(C)C[C@@H]2CCCNc3cccc(n3)S(=O)(=O)NC(=O)c3cnc(-n4ccc(OCCC5C6(CC6)C56CC6)n4)nc3N1C2]

Under nitrogen atmosphere, to (14S)-8-chloro-12,12-dimethyl-2λ⁶-thia-3,7,9,11,18,23-hexaazatetracyclo[17.3.1.111,14.05,10]tetracosa-1(22),5,7,9,19(23),20-hexaene-2,2,4-trione (90 mg, 0.1996 mmol) was added 3-(2-dispiro[2.0.24.13]heptan-7-ylethoxy)-1H-pyrazole (125 mg, 0.6119 mmol), potassium carbonate (58 mg, 0.4197 mmol), cesium carbonate (201 mg, 0.6169 mmol) and then DMSO (0.5 mL). The reaction mixture was heated to 120° C. in an oil bath overnight. The reaction mixture was cooled to RT, filtered, then purified by a reverse phase C₁₈ column and a gradient of 60 to 100% acetonitrile in water (0.1%, formic acid) to provide the desired product (14S)-8-[3-(2-{dispiro[2.0.24.13]heptan-7-yl}ethoxy)-1H-pyrazol-1-yl]-12,12-dimethyl-2λ⁶-thia-3,7,9,11,18,23-hexaazatetracyclo[17.3.1.111,14.05,10]tetracosa-1(22),5,7,9,19(23),20-hexaene-2,2,4-trione (33 mg, 26%): ¹H NMR (400 MHz, DMSO-d₆) δ 12.73 (br. s., 1H), 8.33 (d, J=2.4 Hz, 2H), 7.58 (t, J=7.8 Hz, 1H), 7.07 (d, J=6.8 Hz, 1H), 7.00 (br. s., 1H), 6.72 (d, J=8.6 Hz, 1H), 6.08 (d, J=2.7 Hz, 1H), 4.22 (t, J=6.7 Hz, 2H), 3.98-3.79 (m, 1H), 3.29-3.22 (m, 1H), 2.96 (d, J=13.2 Hz, 1H), 2.75-2.61 (m, 1H), 2.14 (br. s., 1H), 1.89 (dd, J=11.5, 4.4 Hz, 1H), 1.82 (q, J=6.6 Hz, 2H), 1.79-1.70 (m, 1H), 1.68-1.50 (m, 9H), 1.46 (t, J=6.5 Hz, 1H), 1.41-1.28 (m, 1H), 0.86-0.75 (m, 4H), 0.69-0.57 (m, 2H), 0.54-0.43 (m, 2H). ESI-MS m/z calc. 618.2737, found 619.3 (M+1)⁺; Retention time: 4.91 minutes (LC Method F).

### Example 162: Preparation of 12-Benzyl-8-[3-(2-{dispiro[2.0.24.13]heptan-7-yl}ethoxy)-1H-pyrazol-1-yl]-12-methyl-2λ⁶-thia-3,9,11,18,23-pentaazatetracyclo[17.3.1.111,14.05,10]tetracosa-1(23),5(10),6,8,19,21-hexaene-2,2,4-trione (Compound 430) and 12-benzyl-8-[3-(2-{dispiro[2.0.24.13]heptan-7-yl}ethoxy)-1H-pyrazol-1-yl]-12-methyl-2λ⁶-thia-3,9,11,18,23-pentaazatetracyclo[17.3.1.111,14.05,10]tetracosa-1(23),5(10),6,8,19,21-hexaene-2,2,4-trione (Compound 431)

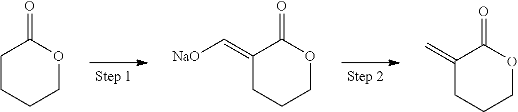

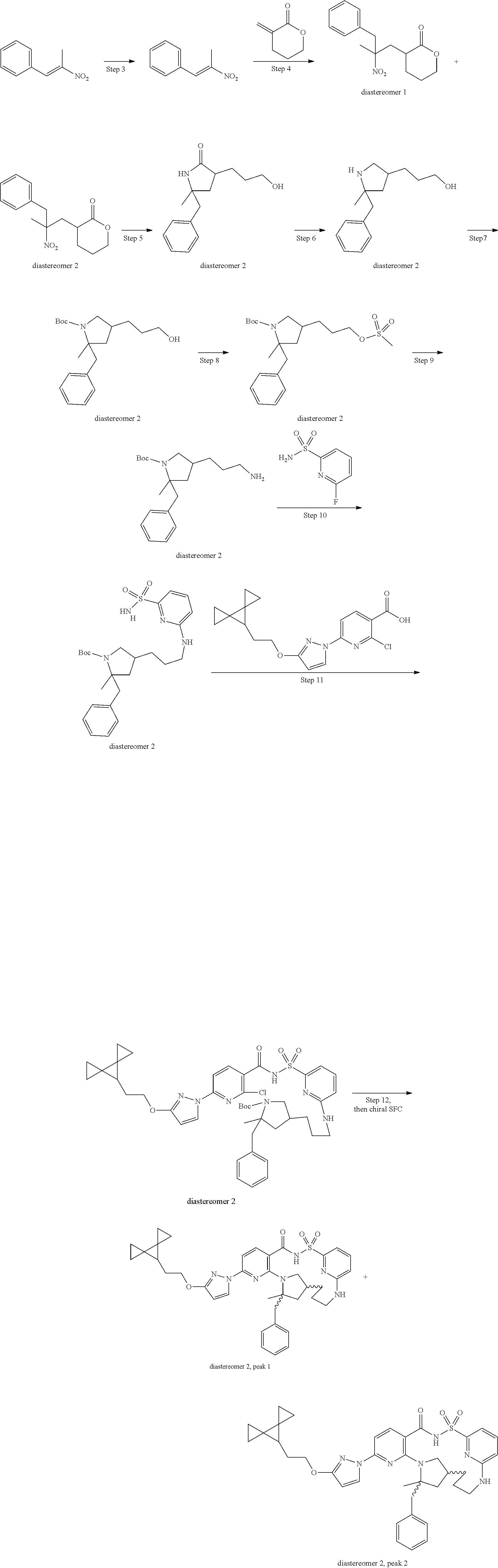

### Step 1: (E)-(2-Oxotetrahydropyran-3-ylidene)methanolate

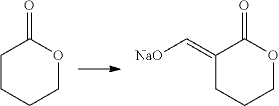

A slurry of 60% sodium hydride (219.72 g, 5.4936 mol) in diethyl ether (5 L) was prepared, then ethanol (23.007 g, 29.160 mL, 499.42 mmol) was added dropwise over the course of 30 min. The resulting mixture was stirred for 15 min, then a mixture of gamma-valerolactone (500 g, 463.39 mL, 4.9942 mol) and ethyl formate (443.95 g, 484.13 mL, 5.9930 mol) was added dropwise over 2.5 hours. The slurry became more viscous as the addition progressed, and was diluted with diethyl ether (10 L) that was added in 1 L portions at 15 min intervals. The resulting mixture was stirred for 20 h at room temperature, then was filtered. The collected solid was dried in a vacuum oven at 20 Torr and at room temperature to give (E)-(2-oxotetrahydropyran-3-ylidene)methanolate (sodium salt) (761 g, 102%) as a white solid.

### Step 2: 3-Methylenetetrahydropyran-2-one

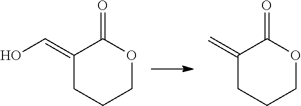

A slurry of (E)-(2-oxotetrahydropyran-3-ylidene)methanolate (sodium salt) (508 g, 3.3842 mol), 4-tert-butylcatechol (50 mg, 0.3008 mmol) and paraformaldehyde (508.07 g, 466.12 mL, 16.921 mol) in THF (6 L) was heated to 65° C. under nitrogen for 21 hours. The resulting white slurry was cooled to room temperature, diluted with ethyl acetate (2 L), then extracted with a saturated solution of sodium bicarbonate (2 L). The phases were separated, and the aqueous phase was extracted with ethyl acetate (2 L). The aqueous phase was discarded, and the combined organic phases were dried over sodium sulfate and concentrated in vacuo to obtain 3-methylenetetrahydropyran-2-one as a yellow oil. The crude product was purified by silica gel chromatography (5-45% ethyl acetate/hexane) to give 3-methylenetetrahydropyran-2-one (187 g, 47%). ESI-MS m/z calc. 112.05243, found 113.4 (M+1)⁺; Retention time: 1.51 minutes (LC method C).

### Step 3: 2-Nitropropylbenzene

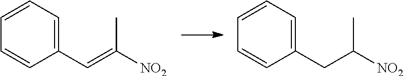

A solution of [(E)-2-nitroprop-1-enyl]benzene (10 g, 61.285 mmol) in 1,4-dioxane (100 mL) was added dropwise to a slurry of sodium borohydride (5.1 g, 5.3968 mL, 134.80 mmol) in a mixture of 1,4-dioxane (100 mL) and ethanol (35 mL). The addition rate was such that the internal temperature did not exceed 30° C. during the course of the addition. The resulting thick white slurry was stirred at room temperature for 1 h, then the reaction was quenched with a 1:1 (v/v) solution of aqueous acetic acid (100 mL). The organic solvents were removed in vacuo and the residue was extracted with chloroform (3×50 mL). The aqueous phase was discarded, and the combined organic phases were extracted with brine (2×25 mL), dried over sodium sulfate, and concentrated in vacuo. The resulting oil was purified by silica gel chromatography (0-25% chloroform/hexane) to give 2-nitropropylbenzene (7.7 g, 73%)¹H NMR (500 MHz, DMSO-d₆) δ 7.33-7.27 (m, 2H), 7.27-7.19 (m, 3H), 5.03-4.93 (m, 1H), 3.21-3.04 (m, 2H), 1.47 (d, J=6.6 Hz, 3H). ESI-MS m/z calc. 165.07898, Retention time: 2.29 minutes (LC Method H).

### Step 4: 3-(2-Methyl-2-nitro-3-phenyl-propyl)tetrahydropyran-2-one

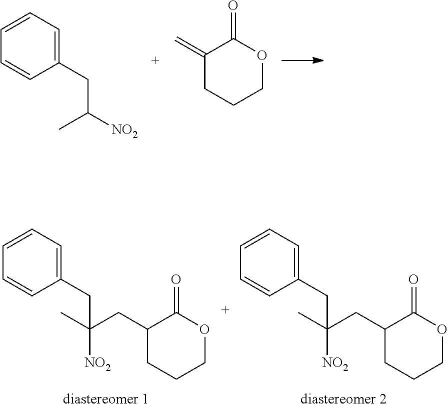

A solution of 3-methylenetetrahydropyran-2-one (15.05 g, 134.22 mmol) dissolved in ACN (150 mL) was added in a slow stream to a mixture of DBU (3.0540 g, 3 mL, 20.061 mmol) and 2-nitropropylbenzene (26.62 g, 161.15 mmol). The reaction is exothermic: the addition rate was such that the internal temperature remained below 35° C. during the course of the addition. The resulting solution was stirred for 6 h at room temperature, then was concentrated under vacuum. The crude material (46.17 g) was purified by silica gel chromatography (330 g SiO₂, 0 to 30% EtOAc/hexanes). Mixed fractions were purified by HPLC using 35 to 85% acetonitrile in water buffered with 0.1% TFA over 60 min.

Diastereomer 1, 3-(2-methyl-2-nitro-3-phenyl-propyl)tetrahydropyran-2-one (978 mg, 2%): ¹H NMR (500 MHz, Chloroform-d) δ 7.34-7.26 (m, 3H), 7.13-7.08 (m, 2H), 4.42-4.24 (m, 2H), 3.45 (d, J=14.1 Hz, 1H), 3.07 (d, J=14.1 Hz, 1H), 3.00 (dd, J=15.3, 5.0 Hz, 1H), 2.78-2.63 (m, 1H), 2.18-2.03 (m, 1H), 1.97-1.88 (m, 3H), 1.53-1.46 (m, 4H). ESI-MS m/z calc. 277.1314, found 278.1 (M+1)⁺; Retention time: 5.01 minutes (LC method C).

Diastereomer 2, 3-(2-methyl-2-nitro-3-phenyl-propyl)tetrahydropyran-2-one (5.26 g, 13%)¹H NMR (500 MHz, Chloroform-d) δ 7.34-7.26 (m, 3H), 7.12-7.00 (m, 2H), 4.33-4.25 (m, 2H), 3.33 (d, J=13.9 Hz, 1H), 3.06 (d, J=13.9 Hz, 1H), 2.65 (dd, J=14.9, 5.2 Hz, 1H), 2.45-2.34 (m, 1H), 2.28 (dd, J=15.0, 5.0 Hz, 1H), 2.11-1.99 (m, 1H), 1.94-1.80 (m, 2H), 1.55-1.49 (m, 4H). ESI-MS m/z calc. 277.1314, found 278.2 (M+1)⁺; Retention time: 5.2 minutes (LC method C).

### Step 5: 5-Benzyl-3-(3-hydroxypropyl)-5-methyl-pyrrolidin-2-one

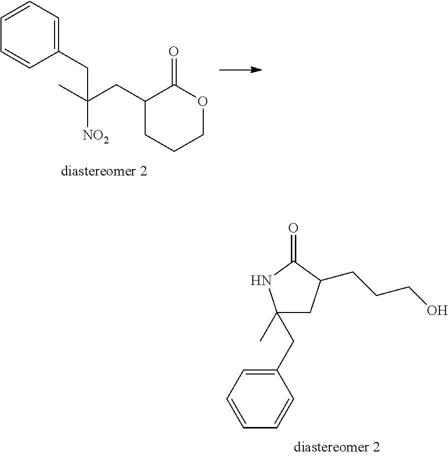 [SMILES: CC(Cc1ccccc1)(CC1CCCOC1=O)[N+](=O)[O-] ; CC1(Cc2ccccc2)CC(CCCO)C(=O)N1]

Diastereomer 2 of 3-(2-methyl-2-nitro-3-phenyl-propyl)tetrahydropyran-2-one (6.4783 g, 22.193 mmol), Raney Nickel (1.0 g, 11.404 mmol), and EtOH (42 mL) were stirred together in a Parr Reactor at 80° C. under hydrogen (3 bar) for 22 h. Celite (10 g) was added and the resulting slurry was filtered. The solids were washed with ethanol (3×50 mL) and discarded. The combined filtrates were concentrated under vacuum to obtain a light brown oil. This was dissolved in DCM (50 mL) and aq. 1 M HCl (10 mL). The layers were separated. The aq. layer was extracted with DCM (50 mL×3). The combined DCM layers were dried over anhydrous sodium sulfate, filtered and concentrated under vacuum. 5-Benzyl-3-(3-hydroxypropyl)-5-methyl-pyrrolidin-2-one (5.99 g, 104%): ¹H NMR (500 MHz, Chloroform-d) δ 7.35-7.18 (m, 3H), 7.19-7.13 (m, 2H), 5.83 (s, 1H), 3.65-3.56 (m, 2H), 2.74 (q, 2H), 2.36-2.27 (m, 1H), 2.20-2.10 (m, 1H), 1.91-1.78 (m, 1H), 1.68-1.50 (m, 3H), 1.46-1.36 (m, 1H), 1.30-1.21 (m, 3H). ESI-MS m/z calc. 247.15723, found 248.3 (M+1)⁺; Retention time: 3.21 minutes (LC method C).

### Step 6: 3-(5-Benzyl-5-methyl-pyrrolidin-3-yl)propan-1-ol

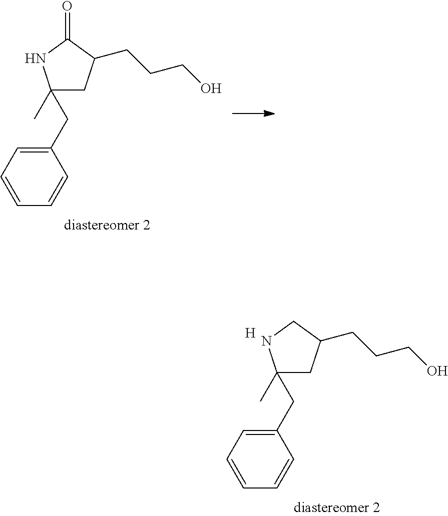

To a solution of 5-benzyl-3-(3-hydroxypropyl)-5-methyl-pyrrolidin-2-one (5.99 g, 23.007 mmol) dissolved in THF (180 mL) at 0° C. was added portion-wise LAH (5.5970 g, 6.1036 mL, 140.09 mmol). The ice-bath was removed and the resulting gray slurry was stirred at 60° C. for 43 h. After cooling to RT, the reaction was cooled to 0° C. H₂O (10 mL) was added slowly and 15% NaOH in H₂O (10 mL) was added slowly. Another 20 mL of H₂O was added. The quenched mixture was diluted with diethyl ether (200 mL). The ice-bath was removed; the mixture was allowed to warm to RT and stir for 30 min. The mixture was filtered through a small column of Celite. The solids were washed with diethyl ether (2×200 mL) and the combined filtrate was concentrated under vacuum. 3-(5-Benzyl-5-methyl-pyrrolidin-3-yl)propan-1-ol (6.87 g, 122%): ¹H NMR (500 MHz, Chloroform-d) δ 7.37-7.09 (m, 5H), 3.61 (t, J=6.5, 6.5 Hz, 2H), 3.23-3.08 (m, 1H), 2.75-2.65 (m, 2H), 2.65-2.56 (m, 1H), 2.18-1.94 (m, 2H), 1.86-1.83 (m, 1H), 1.69-1.47 (m, 4H), 1.45-1.39 (m, 2H), 1.15-1.09 (m, 3H). ESI-MS m/z calc. 233.17796, found 234.2 (M+1)⁺; Retention time: 2.54 minutes (LC method C).

### Step 7: tert-Butyl 2-benzyl-4-(3-hydroxypropyl)-2-methyl-pyrrolidine-1-carboxylate

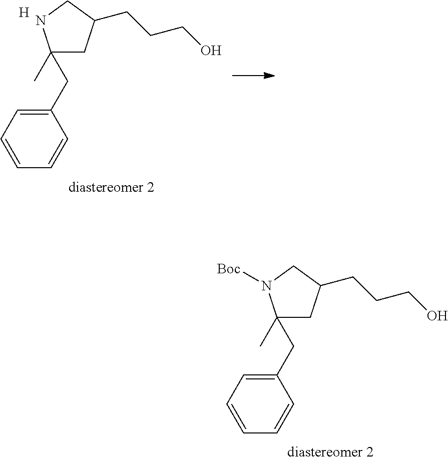

A solution of 3-(5-benzyl-5-methyl-pyrrolidin-3-yl)propan-1-ol (7.6753 g, 32.234 mmol) and tert-butoxycarbonyl tert-butyl carbonate (8.5794 g, 9.0309 mL, 39.311 mmol) in DCM (130 mL) was cooled to 0° C. NaOH (2.7768 g, 69.425 mmol) was dissolved in water (65 mL) and the resulting solution was added to the cooled amino alcohol solution. The ice bath was removed and the reaction mixture was stirred at RT for 19 h. The mixture was transferred to a separatory funnel, the phases were separated and the aq. layer was re-extracted with DCM (50 mL). The combined DCM layers were dried over anhydrous sodium sulfate, filtered and concentrated under vacuum. The crude material (12.82 g, light orange oil) was subjected to silica gel chromatography (120 g SiO₂, eluting with 0 to 45% EtOAc/hexanes). tert-Butyl 2-benzyl-4-(3-hydroxypropyl)-2-methyl-pyrrolidine-1-carboxylate (5.78 g, 51%): ¹H NMR (500 MHz, Chloroform-d) δ 7.28-7.18 (m, 3H), 7.16-7.06 (m, 2H), 3.52-3.47 (m, 2H), 3.41-3.16 (m, 1H), 2.87-2.72 (m, 1H), 2.72-2.62 (m, 1H), 2.21-2.10 (m, 1H), 1.60-1.30 (m, 16H), 1.23-1.09 (m, 3H). ESI-MS m/z calc. 333.2304, found 334.2 (M+1)⁺; Retention time: 5.8 minutes (LC method C).

### Step 8: tert-Butyl 2-benzyl-2-methyl-4-(3-methylsulfonyloxypropyl)pyrrolidine-1-carboxylate

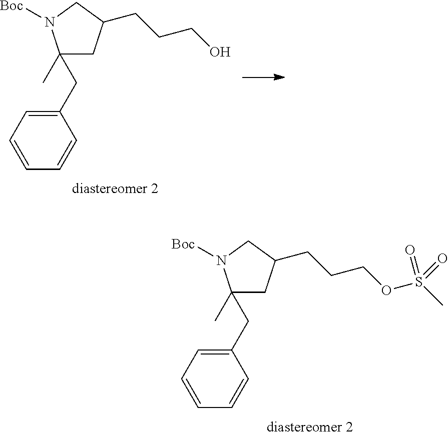 [SMILES: CC(C)(C)OC(=O)N1CC(CCCO)CC1(C)Cc1ccccc1 ; CC(C)(C)OC(=O)N1CC(CCCOS(C)(=O)=O)CC1(C)Cc1ccccc1]

tert-Butyl 2-benzyl-4-(3-hydroxypropyl)-2-methyl-pyrrolidine-1-carboxylate (5.78 g, 16.466 mmol) and triethylamine (3.3396 g, 4.6 mL, 33.003 mmol) were dissolved in DCM (42 mL) and cooled to 0° C. Methanesulfonyl chloride (2.2200 g, 1.5 mL, 19.380 mmol) was added dropwise over 30 min, then the mixture was stirred at RT for 1 h. The reaction was then quenched with aq. sat. sodium bicarbonate (20 mL). The phases were separated and the organic phase was washed with aq. sat. sodium bicarbonate (20 mL), then with H₂O (2×10 mL). The aqueous phases were discarded and the organic phase was dried over anhydrous sodium sulfate, filtered, and concentrated under vacuum to give a light yellow oil. tert-Butyl 2-benzyl-2-methyl-4-(3-methylsulfonyloxypropyl) pyrrolidine-1-carboxylate (7.22 g, 101%): ¹H NMR (500 MHz, Chloroform-d) δ 7.32-7.19 (m, 3H), 7.16-7.05 (m, 2H), 4.17-3.99 (m, 2H), 3.51-3.39 (m, 1H), 3.37-3.19 (m, 1H), 2.99-2.93 (m, 3H), 2.87-2.72 (m, 1H), 2.71-2.62 (m, 1H), 2.19-2.07 (m, 1H), 1.61-1.39 (m, 15H), 1.32-1.17 (m, 3H). ESI-MS m/z calc. 411.20795, found 412.3 (M+1)⁺; Retention time: 6.63 minutes (LC method C).

### Step 9: tert-Butyl 4-(3-aminopropyl)-2-benzyl-2-methyl-pyrrolidine-1-carboxylate

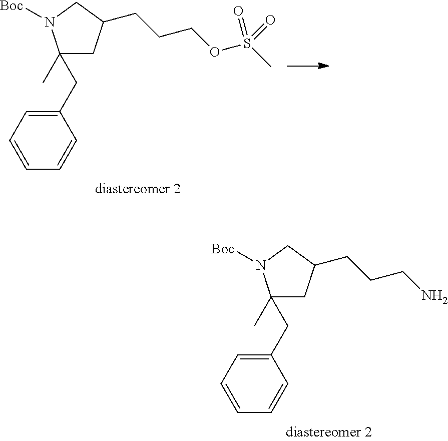 [SMILES: CC(C)(C)OC(=O)N1CC(CCCOS(C)(=O)=O)CC1(C)Cc1ccccc1 ; CC(C)(C)OC(=O)N1CC(CCCN)CC1(C)Cc1ccccc1]

tert-Butyl 2-benzyl-2-methyl-4-(3-methylsulfonyloxypropyl)pyrrolidine-1-carboxylate (7.22 g, 16.666 mmol) was dissolved in dioxane (57 mL) then ammonium hydroxide (51.300 g, 57 mL of 29% w/v, 1.4638 mol) was added. The reaction mixture was stirred at 45° C. for 19 h. Another 29 mL of ammonium hydroxide (29% w/v) was added and the reaction was allowed to stir at 45° C. for 6 h. The mixture was diluted with aq. NaOH (1 M, 20 mL) and extracted with ether (3×60 mL). The combined ether layers were washed with H₂O (2×20 mL), and dried over anhydrous sodium sulfate. The crude was purified by silica gel chromatography (80 g SiO₂, eluting with 0 to 100% EtOAc/hexanes and then with 10% MeOH/DCM buffered with 2% aq. NH₄OH). tert-Butyl 4-(3-aminopropyl)-2-benzyl-2-methyl-pyrrolidine-1-carboxylate (3.13 g, 56%): ¹H NMR (500 MHz, Chloroform-d) δ 7.31-7.16 (m, 3H), 7.16-7.08 (m, 2H), 3.51-3.44 (m, 1H), 3.38 (d, J=13.2 Hz, 1H), 3.35-3.28 (m, 1H), 3.19 (d, J=13.2 Hz, 1H), 2.82 (t, J=9.9, 9.9 Hz, 1H), 2.74 (t, J=9.8, 9.8 Hz, 1H), 2.69 (t, J=13.0, 13.0 Hz, 1H), 2.54 (t, J=6.9, 6.9 Hz, 2H), 2.12 (dd, J=12.5, 5.0 Hz, 1H), 1.52-1.36 (m, 11H), 1.33-1.09 (m, 6H). ESI-MS m/z calc. 332.24637, found 333.4 (M+1)⁺; Retention time: 4.43 minutes (LC method C).

### Step 10: tert-Butyl 2-benzyl-2-methyl-4-[3-[(6-sulfamoyl-2-pyridyl)amino]propyl] pyrrolidine-1-carboxylate

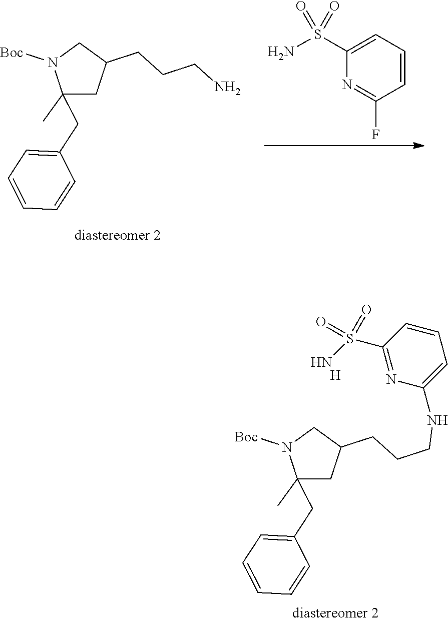

A slurry of tert-butyl 4-(3-aminopropyl)-2-benzyl-2-methyl-pyrrolidine-1-carboxylate (3.13 g, 9.4141 mmol), 6-fluoropyridine-2-sulfonamide (1.8788 g, 10.665 mmol), and sodium carbonate (3.0279 g, 28.568 mmol) in dioxane (20 mL) was heated to 100° C. for 24 h. The mixture was cooled to RT, diluted with H₂O (20 mL), then extracted with diethyl ether (3×20 mL). The aqueous phase was discarded and the combined organic phases were extracted with H₂O (2×10 mL). The aqueous phases were discarded and the organic phase was dried over anhydrous sodium sulfate, filtered, and concentrated under vacuum. The crude was purified by HPLC using 60 to 100% acetonitrile in water buffered with 0.1% TFA. tert-Butyl 2-benzyl-2-methyl-4-[3-[(6-sulfamoyl-2-pyridyl)amino]propyl]pyrrolidine-1-carboxylate (3.2671 g, 68%)¹H NMR (500 MHz, DMSO-d₆) δ 7.55-7.47 (m, 1H), 7.32-7.24 (m, 2H), 7.25-7.17 (m, 1H), 7.13 (d, J=6.8 Hz, 2H), 7.06 (s, 2H), 6.96 (d, J=6.7 Hz, 1H), 6.92-6.83 (m, 1H), 6.63-6.50 (m, 1H), 3.33-3.19 (m, 1H), 3.20-3.08 (m, 2H), 2.78-2.62 (m, 2H), 2.19-2.11 (m, 1H), 1.52-1.44 (m, 9H), 1.42 (s, 2H), 1.38 (s, 2H), 1.34-1.02 (m, 6H). ESI-MS m/z calc. 488.24573, found 489.4 (M+1)⁺; Retention time: 2.81 minutes (LC Method B).

### Step 11: tert-Butyl 2-benzyl-4-[3-[[6-[[2-chloro-6-[3-(2-dispiro[2.0.24.13]heptan-7-ylethoxy)pyrazol-1-yl]pyridine-3-carbonyl]sulfamoyl]-2-pyridyl]amino]propyl]-2-methyl-pyrrolidine-1-carboxylate

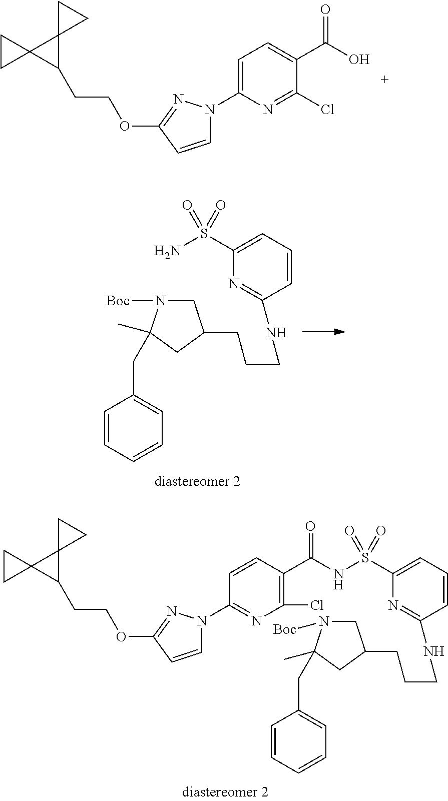 [SMILES: O=C(O)c1ccc(-n2ccc(OCCC3C4(CC4)C34CC4)n2)nc1Cl ; CC(C)(C)OC(=O)N1CC(CCCNc2cccc(S(N)(=O)=O)n2)CC1(C)Cc1ccccc1 ; CC(C)(C)OC(=O)N1CC(CCCNc2cccc(S(=O)(=O)NC(=O)c3ccc(-n4ccc(OCCC5C6(CC6)C56CC6)n4)nc3Cl)n2)CC1(C)Cc1ccccc1]

In a 20-mL vial, 2-chloro-6-[3-(2-dispiro[2.0.24.13]heptan-7-ylethoxy)pyrazol-1-yl]pyridine-3-carboxylic acid (240.3 mg, 0.6011 mmol) was dissolved in THF (5.0 mL), to which CDI (166.1 mg, 1.024 mmol) was added. The resulting mixture was stirred at room temperature for 16 h. After this time, tert-butyl 2-benzyl-2-methyl-4-[3-[(6-sulfamoyl-2-pyridyl)amino]propyl]pyrrolidine-1-carboxylate (295.2 mg, 0.5775 mmol) and DBU (500 μL, 3.343 mmol) were added, and the resulting mixture was stirred at room temperature for 28 h. After this time, 1 N HCl (5 mL) was added, and the mixture was extracted with EtOAc (3×3 mL). The combined organic extracts were washed with H₂O (5 mL) and brine (5 mL), then dried over Na₂SO₄, filtered, and evaporated in vacuo to give a yellow foam. Purification by silica gel chromatography (24 g of silica) using a gradient eluent of 0 to 70% EtOAc in hexanes gave tert-butyl 2-benzyl-4-[3-[[6-[[2-chloro-6-[3-(2-dispiro[2.0.24.13]heptan-7-ylethoxy)pyrazol-1-yl]pyridine-3-carbonyl]sulfamoyl]-2-pyridyl]amino]propyl]-2-methyl-pyrrolidine-1-carboxylate (302.0 mg, 63%) ESI-MS m/z calc. 829.3388, found 830.3 (M+1)⁺; Retention time: 2.3 minutes (LC method G).

### Step 12: 12-Benzyl-8-[3-(2-{dispiro[2.0.24.131heptan-7-yl}ethoxy)-1H-pyrazol-1-yl]-12-methyl-2λ⁶-thia-3,9,11,18,23-pentaazatetracyclo[17.3.1.111,14.05,10]tetracosa-1(23),5(10),6,8,19,21-hexaene-2,2,4-trione (Compound 430) (diastereomer 2, peak 1), 12-benzyl-8-13-(2-{dispiro[2.0.24.13]heptan-7-yl}ethoxy)-1H-pyrazol-1-yl]-12-methyl-2λ⁶-thia-3,9,11,18,23-pentaazatetracyclo[17.3.1.111,14.05,10]tetracosa-1(23),5(10),6,8,19,21-hexaene-2,2,4-trione (Compound 431) (diastereomer 2, peak 2)

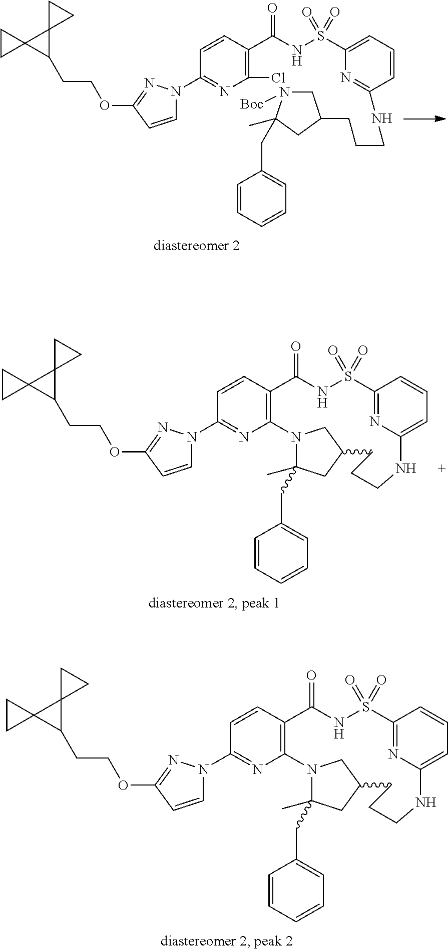 [SMILES: CC(C)(C)OC(=O)N1CC(CCCNc2cccc(S(=O)(=O)NC(=O)c3ccc(-n4ccc(OCCC5C6(CC6)C56CC6)n4)nc3Cl)n2)CC1(C)Cc1ccccc1 ; CC1(Cc2ccccc2)CC2CCCNc3cccc(n3)S(=O)(=O)NC(=O)c3ccc(-n4ccc(OCCC5C6(CC6)C56CC6)n4)nc3N1C2 ; CC1(Cc2ccccc2)CC2CCCNc3cccc(n3)S(=O)(=O)NC(=O)c3ccc(-n4ccc(OCCC5C6(CC6)C56CC6)n4)nc3N1C2]

Stage 1: In a 20-mL vial, tert-butyl 2-benzyl-4-[3-[[6-[[2-chloro-6-[3-(2-dispiro[2.0.24.13]heptan-7-ylethoxy)pyrazol-1-yl]pyridine-3-carbonyl]sulfamoyl]-2-pyridyl]amino]propyl]-2-methyl-pyrrolidine-1-carboxylate (302.0 mg, 0.3637 mmol) was dissolved in DCM (2.0 mL). TFA (200 μL, 2.596 mmol) was added, and the resulting solution was allowed to stand at room temperature for 16 h. More TFA (1.0 mL, 12.98 mmol) was added, and the resulting mixture was stirred at 40° C. for 42 h (incomplete reaction by UPLC). The mixture was then evaporated in vacuo, dissolved in 1:1 MeOH:DMSO (2 mL), filtered, and purified by reverse-phase preparative chromatography using a C₁₈ column and a gradient eluent of 30 to 99% MeCN in H₂O containing 5 mM HCl solution to give recovered starting material (27.8 mg, 9% recovered), as well as an off-white solid, N-[[6-[3-(5-benzyl-5-methyl-pyrrolidin-3-yl)propylamino]-2-pyridyl]sulfonyl]-2-chloro-6-[3-(2-dispiro[2.0.24.13]heptan-7-ylethoxy)pyrazol-1-yl]pyridine-3-carboxamide (hydrochloride salt) (111.8 mg, 40%).

Stage 2: In a 20-mL microwave vial, N-[[6[3-(5-benzyl-5-methyl-pyrrolidin-3-yl)propylamino]-2-pyridyl]sulfonyl]-2-chloro-6-[3-(2-dispiro[2.0.24.13]heptan-7-ylethoxy)pyrazol-1-yl]pyridine-3-carboxamide (hydrochloride salt) (111.8 mg, 0.1458 mmol) was dissolved in NMP (3.0 mL), to which K₂CO₃ (302.8 mg, 2.191 mmol) was added. The resulting mixture was flushed with nitrogen, then stirred at 150° C. for 70 h (incomplete reaction by UPLC). After cooling to room temperature, the resulting mixture was quenched with aqueous HCl solution (1 N; 8 mL), then extracted with EtOAc (3×5 mL). The combined organic extracts were washed with H₂O (2×5 mL) and brine (5 mL), then dried over Na₂SO₄, filtered, and evaporated in vacuo to give 90 mg of a dark brown oil. Purification by silica gel chromatography (24 g of silica) using a gradient eluent of 0 to 50% EtOAc in hexanes gave 55 mg of a white solid that was a mixture of 2 stereoisomers. Separation of the enantiomers was achieved with an SFC purification method using a AS-3 column with a mobile phase of 20-45% MeOH (+NH₃ modifier) in CO₂, and a flow rate of 60 mL/min. Two peaks were obtained and were labeled “Peak 1” and “Peak 2” based on their retention times (Peak 1 is the first to elute and Peak 2 is the second to elute from chiral SFC).

“Diastereomer 2, Peak 1”: 12-benzyl-8-[3-(2-{dispiro[2.0.24.13]heptan-7-yl}ethoxy)-1H-pyrazol-1-yl]-12-methyl-2λ⁶-thia-3,9,11,18,23-pentaazatetracyclo[17.3.1.111,14.05,10]tetracosa-1(23),5(10),6,8,19,21-hexaene-2,2,4-trione (14.4 mg, 6%); ¹H NMR (400 MHz, DMSO-d₆) δ 12.56 (s, 1H), 8.29 (d, J=2.8 Hz, 1H), 7.95-7.73 (m, 1H), 7.64-7.42 (m, 1H), 7.29-7.13 (m, 3H), 7.13-6.81 (m, 5H), 6.76-6.53 (m, 1H), 6.10 (s, 1H), 4.22 (t, J=6.6 Hz, 2H), 3.86-3.58 (m, 2H), 3.16-2.95 (m, 1H), 2.92-2.75 (m, 1H), 2.74-2.60 (m, 1H), 2.06 (d, J=8.1 Hz, 1H), 1.82 (q, J 6.6 Hz, 2H), 1.71-1.56 (m, 1H), 1.55 (s, 3H), 1.47 (t, J=6.5 Hz, 1H), 1.44-1.26 (m, 3H), 1.25-1.03 (m, 3H), 0.90-0.79 (m, 4H), 0.70-0.59 (m, 2H), 0.55-0.43 (m, 2H). ESI-MS m/z calc. ESI-MS m/z calc. 693.30975, found 694.3 (M+1)⁺; Retention time: 2.46 minutes (LC Method A).

“Diastereomer 2, Peak 2”: 12-benzyl-8-[3-(2-{dispiro[2.0.24.13]heptan-7-yl}ethoxy)-1H-pyrazol-1-yl]-12-methyl-2λ⁶-thia-3,9,11,18,23-pentaazatetracyclo[17.3.1.111,14.05,10]tetracosa-1(23),5(10),6,8,19,21-hexaene-2,2,4-trione (14.7 mg, 6%); ESI-MS m/z calc. 693.30975, found 694.3 (M+1)⁺; Retention time: 2.47 minutes (LC Method A).

### Example 163: Preparation of (1³3S)-2⁵5-(3-(2-(dispiro[2.0.2⁴4.1³3]heptan-7-yl)ethoxy)-1H-pyrazol-1-yl)-1⁵5,1⁵5-dimethyl-5-thia-4,7-diaza-6(2,6)-pyridina-1(1,3)-pyrrolidina-2(1,2)-benzenacyclodecaphan-3-one 5,5-dioxide (Compound 432)

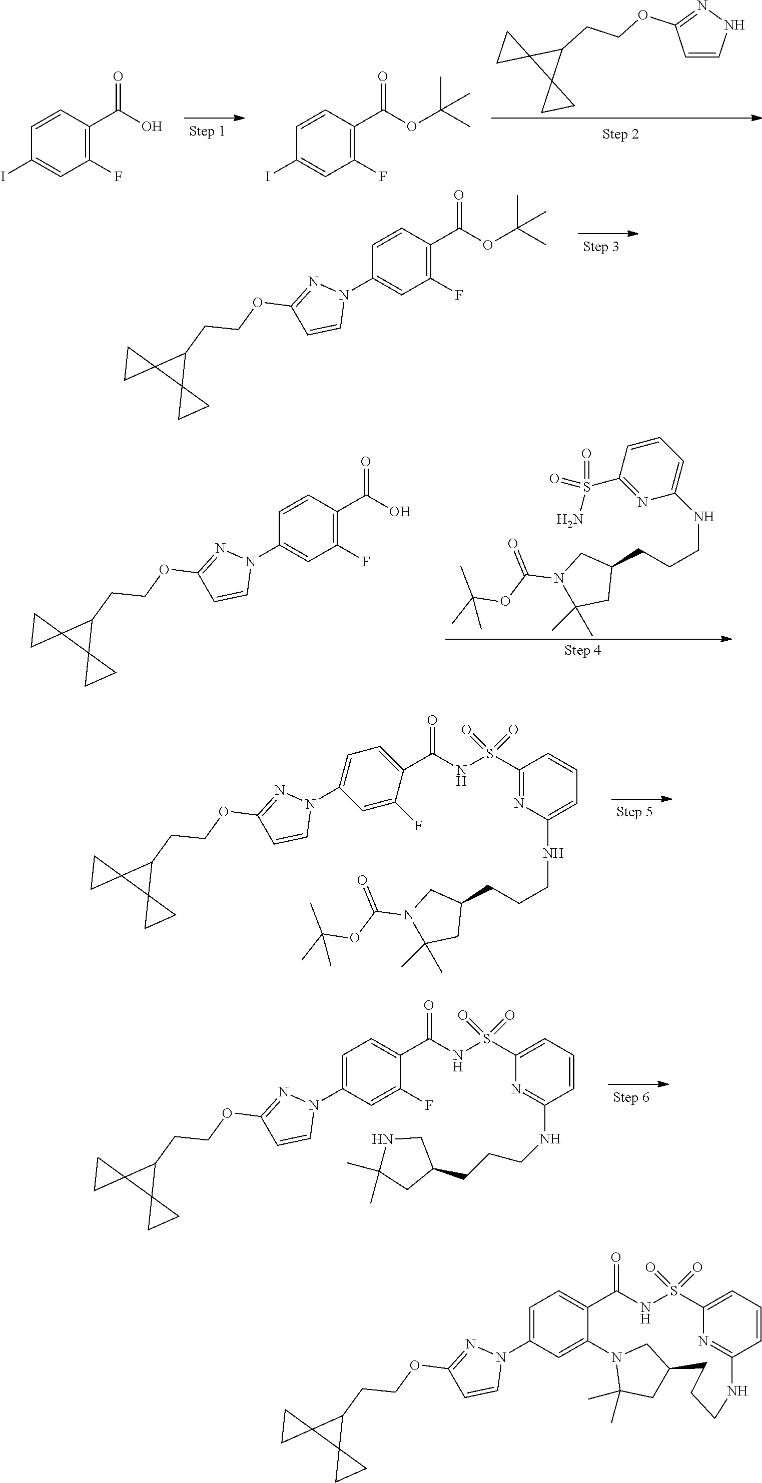

### Step 1: tert-Butyl 2-fluoro-4-iodo-benzoate

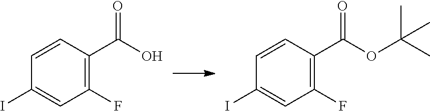

To a solution of 2-fluoro-4-iodo-benzoic acid (1 g, 3.6841 mmol) in THF (6 mL) at room temperature was added tert-butoxycarbonyl tert-butyl carbonate (1 g, 4.5820 mmol), triethylamine (580.80 mg, 0.8 mL, 5.7397 mmol) and DMAP (135 mg, 1.1050 mmol). The reaction mixture was stirred at room temperature for 16 h. The reaction mixture was diluted with EtOAc and washed with water (25 mL). The combined organic layer was washed with brine (25 mL), dried over anhydrous sodium sulfate, filtered and concentrated under reduced pressure. The crude material was purified through silica gel column chromatography using a gradient of 0 to 30% EtOAc in heptanes to afford a light yellow oil, tert-butyl 2-fluoro-4-iodo-benzoate (840 mg, 71%): ¹H NMR (400 MHz, CDCl₃) δ 7.60-7.48 (m, 3H), 1.59 (s, 9H). 19F NMR (377 MHz, CDCl₃) δ −107.75-−107.83 (m, 1F).

### Step 2: tert-Butyl 4-[3-(2-dispiro[2.0.24.13]heptan-7-ylethoxy)pyrazol-1-yl]-2-fluoro-benzoate

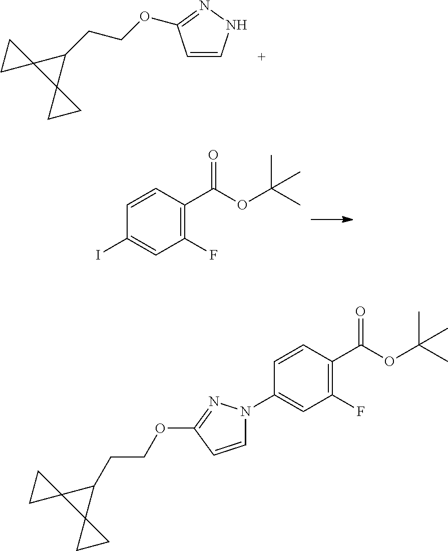 [SMILES: c1cc(OCCC2C3(CC3)C23CC3)n[nH]1 ; CC(C)(C)OC(=O)c1ccc(I)cc1F ; CC(C)(C)OC(=O)c1ccc(-n2ccc(OCCC3C4(CC4)C34CC4)n2)cc1F]

In a reaction tube under nitrogen, tert-butyl 2-fluoro-4-iodo-benzoate (680 mg, 2.1110 mmol), 3-(2-dispiro[2.0.24.13]heptan-7-ylethoxy)-1H-pyrazole (520 mg, 2.5457 mmol), (1S,2S)-(+)-1,2-diaminocyclohexane (125 mg, 1.0947 mmol), potassium carbonate (590 mg, 4.2690 mmol) and copper(I) iodide (85 mg, 0.4463 mmol) were mixed in dioxane (9 mL). The tube was sealed and heated to 100° C. The reaction mixture was stirred at this temperature for 18 h. The reaction mixture was filtered on Celite and the pad was rinsed with EtOAc. The filtrate was concentrated in vacuo and the crude product was purified by reverse phase chromatography on C₁₈ (5 to 100% MeCN in water) to afford tert-butyl 4-[3-(2-dispiro[2.0.24.13]heptan-7-ylethoxy)pyrazol-1-yl]-2-fluoro-benzoate (800 mg, 90%) as a clear oil that became a white solid. ¹H NMR (400 MHz, CDCl₃) δ 7.92 (t, J=8.3 Hz, 1H), 7.76 (d, J=2.7 Hz, 1H), 7.44-7.33 (m, 2H), 5.94 (d, J=2.7 Hz, 1H), 4.26 (t, J=6.7 Hz, 2H), 1.91 (q, J=6.6 Hz, 2H), 1.61 (s, 9H), 1.27 (t, J=7.1 Hz, 1H), 0.90-0.82 (m, 4H), 0.71-0.61 (m, 2H), 0.56-0.46 (m, 2H). ¹⁹F NMR (377 MHz, CDCl₃) δ-106.82 (dd, J=12.3, 8.2 Hz, 1F). ESI-MS m/z calc. 398.20056, found 399.2 (M+1)⁺; Retention time: 2.41 minutes (LC method E).

### Step 3: 4-[3-(2-Dispiro[2.0.24.13]heptan-7-ylethoxy)pyrazol-1-yl]-2-fluoro-benzoic acid

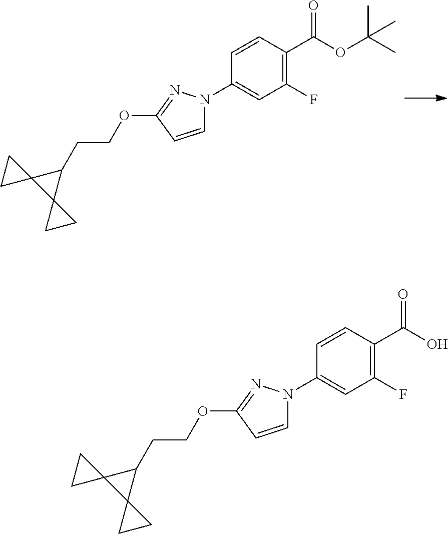 [SMILES: CC(C)(C)OC(=O)c1ccc(-n2ccc(OCCC3C4(CC4)C34CC4)n2)cc1F ; O=C(O)c1ccc(-n2ccc(OCCC3C4(CC4)C34CC4)n2)cc1F]

tert-Butyl 4-[3-(2-dispiro[2.0.24.13]heptan-7-ylethoxy)pyrazol-1-yl]-2-fluoro-benzoate (800 mg, 1.8973 mmol) was dissolved in dichloromethane (15 mL). Then, trifluoroacetic acid (3.4040 g, 2.3 mL, 29.854 mmol) was added dropwise and the reaction was stirred at room temperature for 4 h. The solvent was evaporated to give 4-[3-(2-dispiro[2.0.24.13]heptan-7-ylethoxy)pyrazol-1-yl]-2-fluoro-benzoic acid (trifluoroacetate salt) (810 mg, 90%) as a white solid. ¹H NMR (400 MHz, DMSO-d₆) δ 13.14 (br. s., 1H), 8.49 (d, J=2.7 Hz, 1H), 7.94 (t, J=8.6 Hz, 1H), 7.70-7.62 (m, 2H), 6.12 (d, J=2.7 Hz, 1H), 4.21 (t, J=6.7 Hz, 2H), 1.81 (q, J=6.6 Hz, 2H), 1.46 (t, J=6.4 Hz, 1H), 0.88-0.76 (m, 4H), 0.70-0.60 (m, 2H), 0.53-0.44 (m, 2H). ¹⁹F NMR (377 MHz, DMSO-d₆) δ −107.51 (dd, J=12.3, 8.2 Hz, 1F). ESI-MS m/z calc. 342.13797, found 343.2 (M+1)⁺; Retention time: 1.99 minutes (LC method E).

### Step 4: tert-Butyl (4S)-4-[3-[[6-[[4-[3-(2-dispiro[2.0.24.13]heptan-7-ylethoxy)pyrazol-1-yl]-2-fluoro-benzoyl]sulfamoyl]-2-pyridyl]amino]propyl]-2,2-dimethyl-pyrrolidine-1-carboxylate

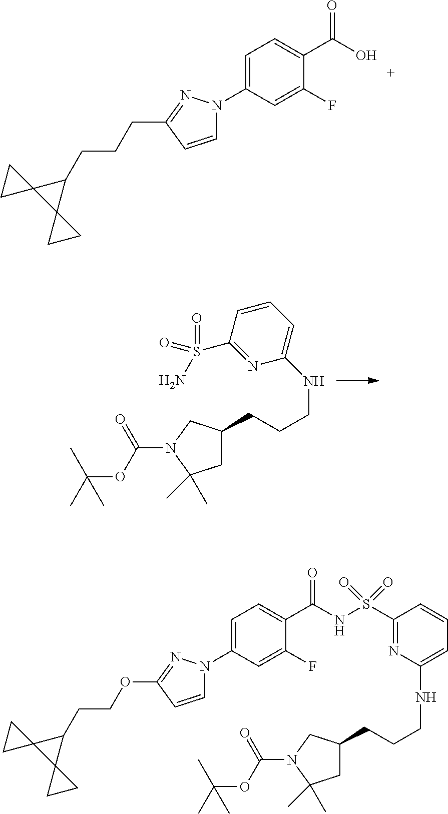 [SMILES: O=C(O)c1ccc(-n2ccc(CCCC3C4(CC4)C34CC4)n2)cc1F ; CC(C)(C)OC(=O)N1C[C@@H](CCCNc2cccc(S(N)(=O)=O)n2)CC1(C)C ; CC(C)(C)OC(=O)N1C[C@@H](CCCNc2cccc(S(=O)(=O)NC(=O)c3ccc(-n4ccc(OCCC5C6(CC6)C56CC6)n4)cc3F)n2)CC1(C)C]

A round bottom flask was charged under nitrogen with 4-[3-(2-dispiro[2.0.24.13]heptan-7-ylethoxy)pyrazol-1-yl]-2-fluoro-benzoic acid (520 mg, 1.4687 mmol) and THF (7 mL). 1,1′-Carbonyldiimidazole (40 mg, 0.2467 mmol) was added and the mixture was stirred under nitrogen at room temperature for 2 h. In a separate flask, a solution of tert-butyl (4S)-2,2-dimethyl-4-[3-[(6-sulfamoyl-2-pyridyl)amino]propyl]pyrrolidine-1-carboxylate (500 mg, 1.2120 mmol) in THF (5 mL) was prepared and it was added via syringe into the activated acid solution. 1,8-Diazabicyclo[5.4.0]undec-7-ene (509.0 mg, 0.5 mL, 3.3435 mmol) was added and the reaction mixture was stirred at room temperature under nitrogen for 18 h. After, the solvents were removed under reduced pressure. The crude was directly purified by reverse phase chromatography eluting with a gradient of MeCN in water (containing 0.1% of formic acid) (5% to 100%) to afford tert-butyl (4S)-4-[3-[[6-[[4-[3-(2-dispiro[2.0.24.13]heptan-7-ylethoxy)pyrazol-1-yl]-2-fluoro-benzoyl]sulfamoyl]-2-pyridyl]amino]propyl]-2,2-dimethyl-pyrrolidine-1-carboxylate (500 mg, 56%) as a light brown solid. ¹H NMR (400 MHz, CDCl₃) δ 8.07-7.93 (m, 1H), 7.81-7.74 (m, 1H), 7.65-7.59 (m, 1H), 7.56-7.46 (m, 2H), 7.41 (d, J=8.6 Hz, 1H), 6.58-6.54 (m, 1H), 5.98-5.92 (m, 1H), 4.80-4.78 (m, 1H), 4.25 (t, J=6.7 Hz, 2H), 3.85-3.74 (m, 1H), 3.41-3.17 (m, 2H), 2.91-2.76 (m, 1H), 2.15-2.04 (m, 1H), 1.95-1.78 (m, 3H), 1.70-1.23 (m, 22H), 0.89-0.81 (m, 4H), 0.72-0.62 (m, 2H), 0.57-0.49 (m, 2H). ¹⁹F NMR (377 MHz, CDCl₃) δ-108.64-−108.96 (m, 1F). ESI-MS m/z calc. 736.34186, found 737.3 (M+1)⁺; Retention time: 2.32 minutes (LC method E).

### Step 5: N-[[6-[3-[(3S)-5,5-Dimethylpyrrolidin-3-yl]propylamino]-2-pyridyl]sulfonyl]-4-[3-(2-dispiro[2.0.24.13]heptan-7-ylethoxy)pyrazol-1-yl]-2-fluoro-benzamide

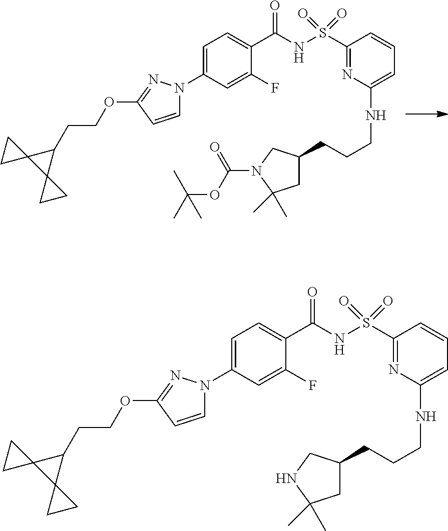 [SMILES: CC(C)(C)OC(=O)N1C[C@@H](CCCNc2cccc(S(=O)(=O)NC(=O)c3ccc(-n4ccc(OCCC5C6(CC6)C56CC6)n4)cc3F)n2)CC1(C)C ; CC1(C)C[C@H](CCCNc2cccc(S(=O)(=O)NC(=O)c3ccc(-n4ccc(OCCC5C6(CC6)C56CC6)n4)cc3F)n2)CN1]

In a round bottom flask, tert-butyl (4S)-4-[3-[[6-[[4-[3-(2-dispiro[2.0.24.13]heptan-7-ylethoxy)pyrazol-1-yl]-2-fluoro-benzoyl]sulfamoyl]-2-pyridyl]amino]propyl]-2,2-dimethyl-pyrrolidine-1-carboxylate (500 mg, 0.6785 mmol) was stirred at room temperature in dichloromethane (5 mL) and 4 M HCl (in dioxane) (2.6 mL, 10.400 mmol) for 2 h. Solvent was evaporated to afford N-[[6-[3-[(3S)-5,5-dimethylpyrrolidin-3-yl]propylamino]-2-pyridyl]sulfonyl]-4-[3-(2-dispiro[2.0.24.13]heptan-7-ylethoxy)pyrazol-1-yl]-2-fluoro-benzamide (hydrochloride salt) (450 mg, 89%) as a light brown solid. ¹H NMR (400 MHz, DMSO-d₆) δ 9.10 (br. s., 1H), 8.96 (br. s., 1H), 8.53 (d, J=2.9 Hz, 1H), 7.76-7.56 (m, 4H), 7.15 (d, J=7.1 Hz, 1H), 6.73 (d, J=8.3 Hz, 1H), 6.13 (d, J=2.9 Hz, 1H), 4.21 (t, J=6.7 Hz, 2H), 3.56 (s, 6H), 3.34-3.13 (m, 3H), 1.88-1.75 (m, 3H), 1.48-1.19 (m, 9H), 0.86-0.79 (m, 4H), 0.68-0.61 (m, 2H), 0.52-0.46 (m, 2H). ¹⁹F NMR (377 MHz, DMSO-d₆) δ −109.84 (dd, J=12.3, 8.2 Hz, 1F). ESI-MS m/z calc. 636.2894, found 637.2 (M+1)⁺; Retention time: 1.62 minutes (LC method E).

### Step 6: (1³3S)-2⁵5-(3-(2-(dispiro[2.0.2⁴4.1³3]heptan-7-yl)ethoxy)-1H-pyrazol-1-yl)-1⁵5,1⁵5-dimethyl-5-thia-4,7-diaza-6(2,6)-pyridina-1(1,3)-pyrrolidina-2(1,2)-benzenacyclodecaphan-3-one 5,5-dioxide (Compound 432)

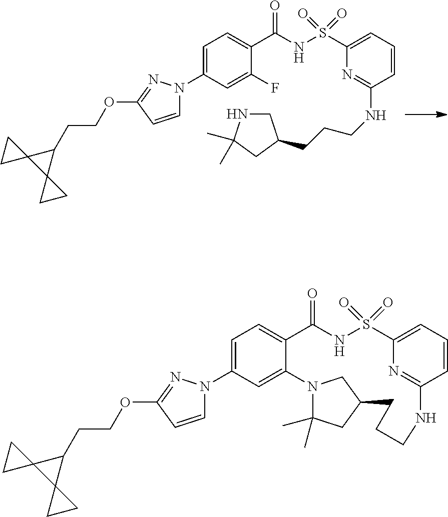 [SMILES: CC1(C)C[C@H](CCCNc2cccc(S(=O)(=O)NC(=O)c3ccc(-n4ccc(OCCC5C6(CC6)C56CC6)n4)cc3F)n2)CN1 ; CC1(C)C[C@@H]2CCCNc3cccc(n3)S(=O)(=O)NC(=O)c3ccc(-n4ccc(OCCC5C6(CC6)C56CC6)n4)cc3N1C2]

N-[[6-[3-[(3S)-5,5-Dimethylpyrrolidin-3-yl]propylamino]-2-pyridyl]sulfonyl]-4-[3-(2-dispiro[2.0.24.13]heptan-7-ylethoxy)pyrazol-1-yl]-2-fluoro-benzamide (hydrochloride salt) (160 mg, 0.2144 mmol) was dissolved in DMSO (3.2 mL). Cesium carbonate (560 mg, 1.7188 mmol) was added. The reaction tube was sealed and the mixture was heated at 150° C. for 4 days. The reaction mixture was directly purified by C₁₈ reverse phase chromatography using gradients of 5 to 100% acetonitrile in water to give (1³3 S)-2⁵5-(3-(2-(dispiro[2.0.2⁴4.1³3]heptan-7-yl)ethoxy)-1H-pyrazol-1-yl)-1⁵5,1⁵5-dimethyl-5-thia-4,7-diaza-6(2,6)-pyridina-1(1,3)-pyrrolidina-2(1,2)-benzenacyclodecaphan-3-one 5,5-dioxide (40 mg, 29%) as a white solid. ¹H NMR (400 MHz, DMSO-d₆) δ 12.66 (br. s., 1H), 8.29 (d, J=2.4 Hz, 1H), 7.57 (t, J=7.7 Hz, 1H), 7.46-7.39 (m, 2H), 7.09-6.94 (m, 3H), 6.69 (d, J=8.6 Hz, 1H), 6.01 (d, J=2.7 Hz, 1H), 4.19 (t, J=6.6 Hz, 2H), 3.89-3.85 (m, 1H), 3.17-3.13 (m, 1H), 3.00-2.81 (m, 2H), 2.23-2.17 (m, 1H), 1.93-1.89 (m, 1H), 1.82 (q, J=6.7 Hz, 2H), 1.74-1.56 (m, 3H), 1.53 (s, 4H), 1.46 (t, J=6.6 Hz, 1H), 1.38 (s, 3H), 1.33-1.28 (m, 1H), 0.85-0.75 (m, 4H), 0.66-0.60 (m, 2H), 0.51-0.44 (m, 2H). ESI-MS m/z calc. 616.2832, found 617.3 (M+1)⁺; Retention time: 3.47 minutes; LCMS Method: XBridge C₁₈ 4.6×75 mm 5 μm, Initial Gradient at 95% NH₄HCO₃/5% MeCN 6 min run with 1 min equilibration Gradient 0 to 3 min at 95% MeCN and hold for 3 minutes. Flow 1.5 mL/min.

### Example 164: Preparation of (14S)-8-[3-(2-{Dispiro[2.0.24.13]heptan-7-yl}ethoxy)-1H-pyrazol-1-yl]-12,12-dimethyl-2λ⁶-thia-3,9,11,17,23-pentaazatetracyclo[17.3.1.111,14.05,10]tetracosa-1(23),5,7,9,19,21-hexaene-2,2,4-trione (Compound 433)

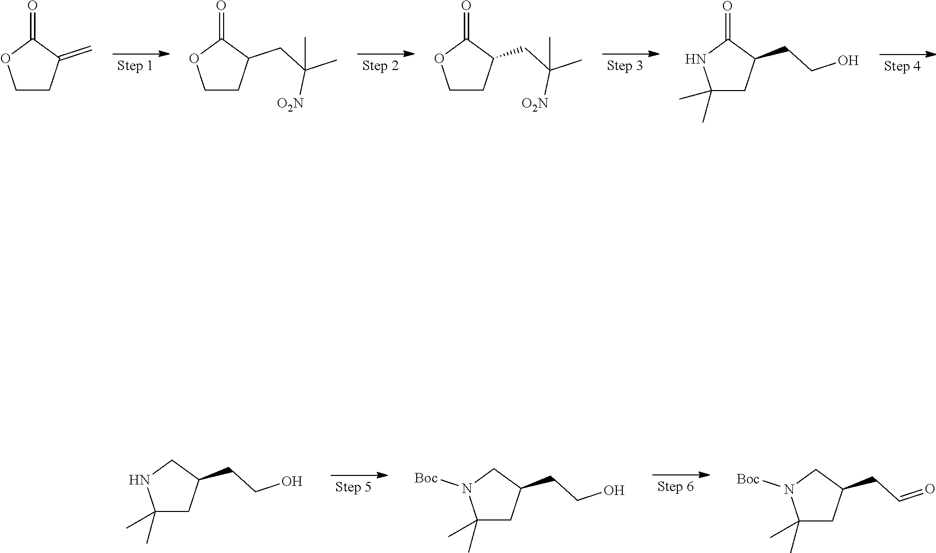

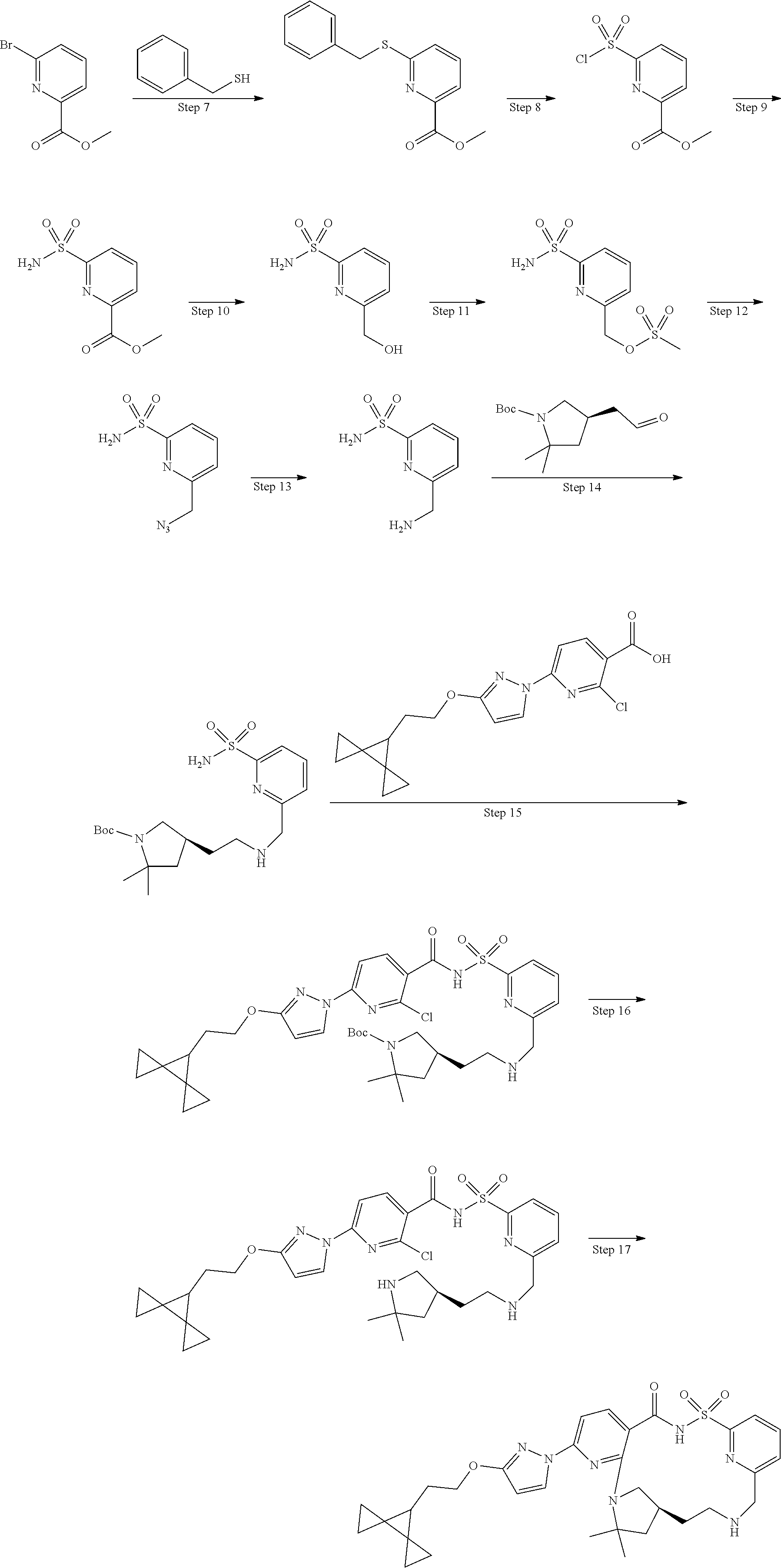

### Step 1: 3-(2-Methyl-2-nitro-propyl)tetrahydrofuran-2-one

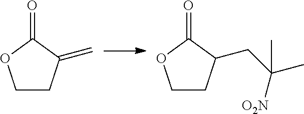

A solution of 3-methylenetetrahydrofuran-2-one (101.11 g, 90.357 mL, 989.46 mmol) dissolved in acetonitrile (1.1 L) was added in a slow stream to a mixture of DBU (23.414 g, 23 mL, 153.80 mmol) and 2-nitropropane (107.14 g, 108 mL, 1.2026 mol). The reaction is exothermic: the addition rate was such that the internal temperature remained below 35° C. during the course of the addition. The resulting solution was stirred for 19 h at room temperature, then was concentrated under vacuum to obtain a light yellow solid. The crude product was stirred overnight in diethyl ether (500 mL), then filtered. The solids were dissolved in DCM (500 mL) and washed with 3.0 M aq. HCl (2×500 mL), H₂O (500 mL), sat. aq. NaHCO₃(500 mL), and sat. aq. NaCl (500 mL). The DCM layer was dried over anhydrous magnesium sulfate, filtered and concentrated under vacuum to give white crystal solids. 3-(2-Methyl-2-nitro-propyl)tetrahydrofuran-2-one (157.59 g, 82%): ¹H NMR (500 MHz, DMSO-d₆) δ 4.27 (td, J=8.6, 8.6, 1.5 Hz, 1H), 4.15-4.00 (m, 1H), 2.68 (dddd, J=11.4, 9.7, 8.5, 2.9 Hz, 1H), 2.43 (dd, J=14.8, 2.9 Hz, 1H), 2.34-2.23 (m, 1H), 2.09-1.99 (m, 1H), 1.85 (qd, J=11.6, 11.6, 11.5, 8.6 Hz, 1H), 1.59 (s, 3H), 1.58 (s, 3H). ESI-MS m/z calc. 187.08446, found 188.2 (M+1)⁺; Retention time: 1.39 minutes (LC Method B).

### Step 2: (3R)-3-(2-Methyl-2-nitro-propyl)tetrahydrofuran-2-one and (3S)-3-(2-methyl-2-nitro-propyl)tetrahydrofuran-2-one

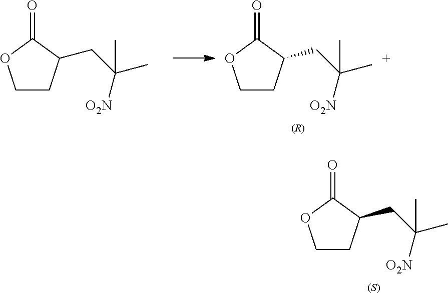

A racemic mixture of 3-(2-methyl-2-nitro-propyl)tetrahydrofuran-2-one (173.55 g, 927.1 mmol) was separated by Chiral SFC, using an AD-H (2×25 cm) column, and 35% methanol in CO₂, 100 bar as the eluent. The flow rate was 70 mL/min (220 nm) and the injection volume was 3 mL, of a 20 mg/mL solution in methanol:DCM. The two isomers were isolated as solids after drying cold with the addition of acetonitrile to prevent concentration in methanol.

Peak 1, (3R)-3-(2-Methyl-2-nitro-propyl)tetrahydrofuran-2-one (88 g, 50%). ¹H NMR (400 MHz, DMSO-d₆) δ 4.27 (td, J=8.6, 1.5 Hz, 1H), 4.09 (ddd, J=10.9, 8.7, 6.1 Hz, 1H), 2.76-2.63 (m, 1H), 2.43 (dd, J=14.8, 2.9 Hz, 1H), 2.35-2.24 (m, 1H), 2.04 (dd, J=14.8, 10.0 Hz, 1H), 1.94-1.79 (m, 1H), 1.59 (s, 3H), 1.58 (s, 3H). ESI-MS m/z calc. 187.08446, Retention time: 0.71 minutes (mass not detected) (LC Method A).

Peak 2, (3S)-3-(2-Methyl-2-nitro-propyl)tetrahydrofuran-2-one (80 g, 46%). ¹H NMR (400 MHz, DMSO-d₆) δ 4.27 (td, J=8.6, 1.5 Hz, 1H), 4.09 (ddd, J=10.9, 8.7, 6.1 Hz, 1H), 2.76-2.62 (m, 1H), 2.43 (dd, J=14.8, 2.9 Hz, 1H), 2.33-2.23 (m, 1H), 2.04 (dd, J=14.8, 10.0 Hz, 1H), 1.91-1.78 (m, 1H), 1.59 (s, 3H), 1.58 (s, 3H). ESI-MS m/z calc. 187.08446, Retention time: 0.76 minutes (mass not detected) (LC Method A).

### Step 3: (3R)-3-(2-Hydroxyethyl)-5,5-dimethyl-pyrrolidin-2-one

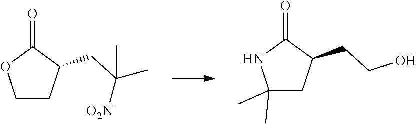

To a solution of (3R)-3-(2-methyl-2-nitro-propyl)tetrahydrofuran-2-one (37 g, 197.66 mmol) in ethanol (370.00 mL) was added Raney Nickel (8 g, 91.230 mmol) in a Parr reactor. The reactor was stirred at 80° C. under hydrogen (3 bar) for 48 h. Celite (10 g) was added and the resulting slurry was filtered. The solids were washed with ethanol (2×150 mL) and discarded. The combined filtrates were concentrated under vacuum to obtain a yellow solid. The solid was then triturated with ether overnight and the resulting solid was collected to yield (3R)-3-(2-hydroxyethyl)-5,5-dimethyl-pyrrolidin-2-one (26.9 g, 82%) as a white solid. ¹H NMR (500 MHz, DMSO-d₆) δ 7.68 (s, 1H), 4.54 (m, 1H), 3.47 (m, 1H), 3.41 (m, 1H), 2.48-2.42 (m, 1H), 2.04 (m, 1H), 1.86 (m, 1H), 1.42 (m, 1H), 1.30 (m, 1H), 1.18 (s, 3H), 1.13 (s, 3H).

### Step 4: 2-[(3R)-5,5-Dimethylpyrrolidin-3-yl]ethanol

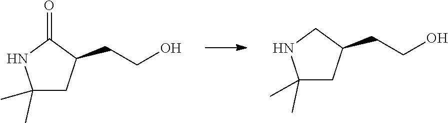

LAH (40 g, 1.0539 mol) was added in small portions to a cooled (0° C.) solution of (3R)-3-(2-hydroxyethyl)-5,5-dimethyl-pyrrolidin-2-one (31.5 g, 200.37 mmol) in THF (350 mL). The resulting slurry was allowed to warm to room temperature and stirred for 1 h. It was then heated to 70° C. for 48 h. After cooling to room temperature, the reaction was cooled to 0° C. using an ice bath. Water (40 mL) was slowly added, followed by 15% w/v aq. NaOH solution (40 mL). Another 120 mL of water was added and the ice bath was removed. The quenched reaction mixture was diluted with diethyl ether (200 mL) and was allowed to warm and stir at room temperature for 30 min. The mixture was filtered through Celite and the solids were washed with diethyl ether (2×100 mL). The combined filtrates were then dried over anhydrous sodium sulfate and filtered. The combined filtrate was evaporated in vacuo to give 2-[(3R)-5,5-dimethylpyrrolidin-3-yl]ethanol (32 g, 100%) as a yellowish oil. ¹H NMR (500 MHz, DMSO-d₆) δ 4.40 (s, 1H), 3.46-3.39 (m, 2H), 2.97-2.90 (m, 1H), 2.44-2.32 (m, 1H), 2.19-2.00 (m, 1H), 1.71-1.62 (m, 1H), 1.49 1.35 (m, 2H), 1.09-0.97 (m, 7H). [Note: 1H is missing from the overall count of 17 protons from the product (C₈H₁₇NO).]

### Step 5: tert-Butyl (4R)-4-(2-hydroxyethyl)-2,2-dimethyl-pyrrolidine-1-carboxylate

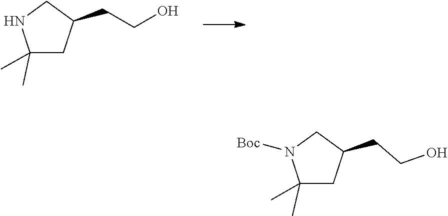

A solution of 2-[(3R)-5,5-dimethylpyrrolidin-3-yl]ethanol (32 g, 203.31 mmol) in DCM (160.00 mL) was cooled to 0° C., then a solution of NaOH (19 g, 475.03 mmol) in H₂O (160 mL) was added. A solution of tert-butoxycarbonyl tert-butyl carbonate (59 g, 62.105 mL, 270.34 mmol) in DCM (160 mL) was added dropwise to the chilled amine mixture, then the ice water bath was removed and the reaction was allowed to stir at room temperature for 22 h. The phases were then separated: the aqueous phase was extracted with DCM (150 mL). The organic phase was dried over sodium sulfate and concentrated in vacuo, then purified by silica gel chromatography using 3% MeOH in DCM to give tert-butyl (4R)-4-(2-hydroxyethyl)-2,2-dimethyl-pyrrolidine-1-carboxylate (30.915 g, 60%) as a clear oil. ¹H NMR (500 MHz, DMSO-d₆) δ 4.42 (t, J=5.1, 1H), 3.56 (q, J=8.7 Hz, 1H), 3.42-3.36 (m, 2H), 2.79 (dt, J=12.8, 10.6 Hz, 1H), 2.21-2.10 (m, 1H), 1.92 1.84 (m, 1H), 1.47-1.44 (m, 2H), 1.37 (m, 13H), 1.24 (s, 3H). ESI-MS m/z calc. 243.18344, found 244.5 (M+1)⁺; Retention time: 2.17 minutes (LC Method H).

### Step 6: tert-Butyl (4R)-2,2-dimethyl-4-(2-oxoethyl)pyrrolidine-1-carboxylate

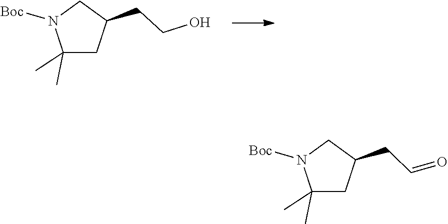 [SMILES: CC(C)(C)OC(=O)N1C[C@@H](CCO)CC1(C)C ; CC(C)(C)OC(=O)N1C[C@@H](CC=O)CC1(C)C]

A buffered solution of bleach was prepared by dissolving sodium bicarbonate (9.53 g, 113.44 mmol) into a solution of aqueous sodium hypochlorite (136 mL of 1.6 M, 217.60 mmol) and stirring at in an ice bath. That solution was then added dropwise to a second solution stirred mechanically, that was prepared in advance by adding sodium bromide (944 mg, 9.1745 mmol) (dissolved in water (5 mL) and TEMPO (73 mg, 0.4672 mmol) to tert-butyl (4R)-4-(2-hydroxyethyl)-2,2-dimethyl-pyrrolidine-1-carboxylate (43.68 g, 179.50 mmol) in DCM (570 mL) maintained at −12° C. During the addition of the buffered bleach solution to the substrate solution the internal temperature was maintained below −10° C. After completion of the addition, the reaction mixture was maintained at −14° C. for 10 min. The excess bleach was quenched with ethanol (2.3670 g, 3 mL, 51.380 mmol). The reaction mixture was concentrated under reduced pressure at 35° C. The resulting residue was partitioned between ethyl acetate (200 mL) and water (100 mL). The aqueous phase was separated and washed with ethyl acetate (100 mL). The aqueous phase was kept aside. The organics were combined, washed with brine (150 mL), dried with sodium sulfate, filtered and concentrated under reduced pressure to provide tert-butyl (4R)-2,2-dimethyl-4-(2-oxoethyl)pyrrolidine-1-carboxylate (34.6 g, 69%)¹H NMR (400 MHz, CDCl₃) δ 9.78 (s, 1H), 3.87-3.70 (m, 1H), 3.01-2.82 (m, 1H), 2.68-2.41 (m, 3H), 2.07-1.94 (m, 1H), 1.53-1.30 (m, 16H). ESI-MS m/z calc. 241.1678, found 186.2 (M-tBu+2H=M−55)⁺; Retention time: 1.79 minutes (LC method E).

### Step 7: Methyl 6-benzylsulfanylpyridine-2-carboxylate

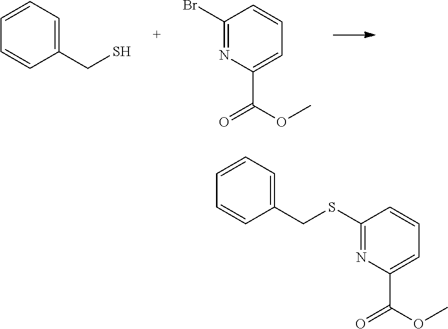

A solution of phenylmethanethiol (55.70 g, 52.647 mL, 443.97 mmol) in THF (1250 mL) was purged with nitrogen. The solution was stirred at 0° C. to which was added sodium hydride (22.58 g, 564.55 mmol) in mineral oil portion-wise. After addition, the mixture was warmed to RT, and stirred under nitrogen for 0.5 h. Then, methyl 6-bromopyridine-2-carboxylate (100.82 g, 457.36 mmol) was added portion-wise and the reaction was stirred under nitrogen at RT for 1.5 h. The reaction was quenched with H₂O (600 mL) and extracted with EtOAc (3×500 mL). The combined EtOAc layers were washed with sat. aq. NaCl (500 mL), dried over anhydrous sodium sulfate, filtered, and concentrated under vacuum to give methyl 6-benzylsulfanylpyridine-2-carboxylate (112.47 g, 90%): ESI-MS m/z calc. 259.0667, found 260.2 (M+1)⁺; Retention time: 2.95 minutes (LC Method B).

### Step 8: Methyl 6-chlorosulfonylpyridine-2-carboxylate

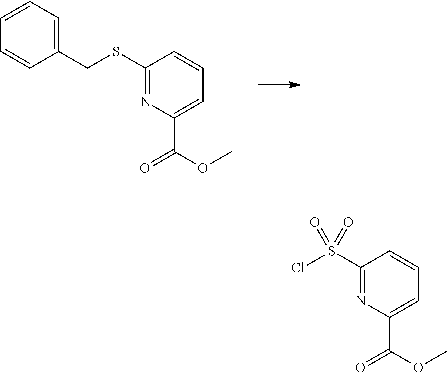 [SMILES: COC(=O)c1cccc(SCc2ccccc2)n1 ; COC(=O)c1cccc(S(=O)(=O)Cl)n1]

A solution of methyl 6-benzylsulfanylpyridine-2-carboxylate (112.47 g, 412.02 mmol) in DCM (850 mL) and water (260 mL) was cooled to 0° C., and with vigorous stirring, sulfuryl chloride (228.14 g, 140 mL, 1.6396 mol) was added drop-wise while the temperature remained below 5° C. The organic phase was separated, washed with H₂O (2×300 mL), dried over anhydrous sodium sulfate, filtered and concentrated under vacuum. The residue (117.47 g) was dissolved in DCM (100 mL). Hexanes (500 mL) was added and approximately 300 mL of solvent was evaporated. The resulting white precipitate was filtered under vacuum and the solids were washed with hexanes (2×300 mL). The filtrate was concentrated under vacuum and dissolved in DCM (50 mL). Hexanes (250 mL) was added and the DCM was slowly evaporated. Approximately 150 mL of solvent was evaporated and collected before the white precipitate was filtered under vacuum. The solids were washed with hexanes (2×80 mL). A light yellow solid, methyl 6-chlorosulfonylpyridine-2-carboxylate (67.70 g, 66%), resulted. ¹H NMR (500 MHz, Chloroform-d) δ 8.51-8.44 (m, 1H), 8.32-8.26 (m, 1H), 8.22 (t, J=7.8, 7.8 Hz, 1H), 4.06 (s, 3H).

### Step 9: Methyl 6-sulfamoylpyridine-2-carboxylate

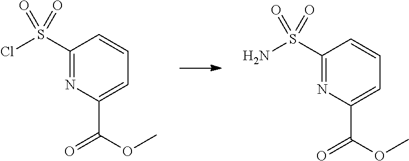

To a solution of methyl 6-chlorosulfonylpyridine-2-carboxylate (67.6 g, 284.00 mmol) in ACN (1100 mL) cooled to 0° C. was added dropwise concentrated aq. ammonium hydroxide (45 mL of 12 M, 540.00 mmol). The reaction was exothermic so the dropwise addition was kept at a rate maintaining the internal temperature below 5° C. Near the end of addition, the internal temperature decreased as more ammonium hydroxide was added dropwise. The reaction was stirred at 0° C. for 15 min. After consumption of the starting material, the reaction mixture was diluted with H₂O (500 mL), and extracted with EtOAc (3×300 mL). The combined organic layers were dried over anhydrous sodium sulfate, filtered and concentrated under vacuum to give a white solid: methyl 6-sulfamoylpyridine-2-carboxylate (60.49 g, 93%); ¹H NMR (500 MHz, DMSO-d₆) δ 8.30-8.22 (m, 2H), 8.19-8.13 (m, 1H), 7.63 (s, 2H), 3.92 (s, 3H). ESI-MS m/z calc. 216.02048, found 217.3 (M+1)⁺; Retention time: 1.31 minutes (LC Method B).

### Step 10: 6-(Hydroxymethyl)pyridine-2-sulfonamide

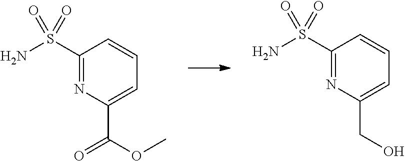

To a solution of methyl 6-sulfamoylpyridine-2-carboxylate (60.5 g, 263.03 mmol) in THF (450 mL) was added a solution of calcium chloride (58.3 g, 525.30 mmol) in anhydrous ethanol (450 mL). Sodium borohydride (30 g, 792.97 mmol) was then added portion-wise. The reaction was then allowed to stir for 24 h at room temperature. The reaction was quenched with NaHCO₃(200 mL) to achieve pH 8. The mixture was then filtered and the filtrate was partitioned with EtOAc (200 mL). The aqueous layer was extracted with EtOAc (10×250 mL). The organic layers were combined, dried with anhydrous sodium sulfate, filtered, and concentrated under reduced pressure to yield 6-(hydroxymethyl)pyridine-2-sulfonamide (44.6 g, 86%) as a yellow solid. ¹H NMR (500 MHz, DMSO-d₆) δ 8.04 (t, J=7.7, 7.7 Hz, 1H), 7.77 (d, J=7.6 Hz, 1H), 7.68 (d, J=7.8 Hz, 1H), 7.40 (s, 2H), 5.59 (t, J=5.8, 5.8 Hz, 1H), 4.62 (d, J=6.0 Hz, 2H). ESI-MS m/z calc. 188.02556, found 189.1 (M+1)⁺; Retention time: 0.34 minutes (LC Method H).

### Step 11: (6-Sulfamoyl-2-pyridyl)methyl methanesulfonate

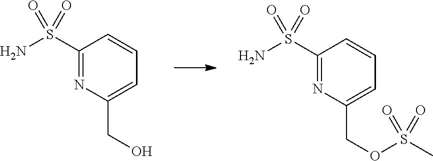 [SMILES: NS(=O)(=O)c1cccc(CO)n1 ; CS(=O)(=O)OCc1cccc(S(N)(=O)=O)n1]

Into a suspension of 6-(hydroxymethyl)pyridine-2-sulfonamide (36.5 g, 174.54 mmol) in THF (600 mL) was added triethylamine (36.300 g, 50 mL, 358.73 mmol). Then, methanesulfonyl chloride (17.760 g, 12 mL, 155.04 mmol) was added to the reaction mixture dropwise at 0° C. The reaction was stirred at 0° C. for 1 h. The reaction was diluted with sat. sodium bicarbonate (200 mL), filtered, and extracted with ethyl acetate (3×150 mL). The combined organic layers were washed with brine, dried over anhydrous sodium sulfate and concentrated under vacuum. The residue was purified by silica gel chromatography using 0 to 10% methanol in DCM to furnish (6-sulfamoyl-2-pyridyl)methyl methanesulfonate (19 g, 39%) as a beige solid. ESI-MS m/z calc. 266.0031, found 267.0 (M+1)⁺; Retention time: 1.31 minutes (LC Method B).

### Step 12: Diazonio-[(6-sulfamoyl-2-pyridyl)methyl]azanide

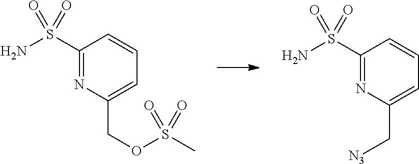 [SMILES: CS(=O)(=O)OCc1cccc(S(N)(=O)=O)n1 ; [N-]=[N+]=NCc1cccc(S(N)(=O)=O)n1]

To a solution of (6-sulfamoyl-2-pyridyl)methyl methanesulfonate (19 g, 67.782 mmol) in anhydrous DMF (225 mL) was added sodium azide (8.8 g, 135.36 mmol). The solution was stirred for 2 h at RT. The reaction was then diluted with EtOAc (400 mL) and saturated sodium bicarbonate (200 mL). The white solids were filtered then the aqueous layer was extracted with EtOAc (2×100 mL). The organic layers were then combined, washed with brine, dried with anhydrous sodium sulfate, filtered, and concentrated under reduced pressure diazonio-[(6-sulfamoyl-2-pyridyl)methyl]azanide (12.3 g, 80%) as a beige solid. ESI-MS m/z calc. 213.03204, found 214.1 (M+1)⁺; Retention time: 1.38 minutes (LC Method B).

### Step 13: 6-(Aminomethyl)pyridine-2-sulfonamide

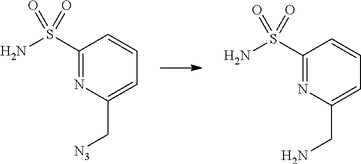 [SMILES: [N-]=[N+]=NCc1cccc(S(N)(=O)=O)n1 ; NCc1cccc(S(N)(=O)=O)n1]

To a solution of diazonio-[(6-sulfamoyl-2-pyridyl)methyl]azanide (7.05 g, 31.081 mmol) in MeOH (2.75 L) was added palladium on carbon (1.5 g, 14.095 mmol). The reaction was stirred at room temperature for 2 h under an atmosphere of hydrogen. The catalyst was filtered off through a pad of Celite and the filtrate was concentrated under reduced pressure to yield 6-(aminomethyl)pyridine-2-sulfonamide (5.6 g, 77%) as a yellow solid. ESI-MS m/z calc. 187.0415, found 187.9 (M+1)⁺; Retention time: 0.87 minutes (LC method B).

### Step 14: tert-Butyl (4S)-2,2-dimethyl-4-[2-[(6-sulfamoyl-2-pyridyl)methylamino] ethyl]pyrrolidine-1-carboxylate

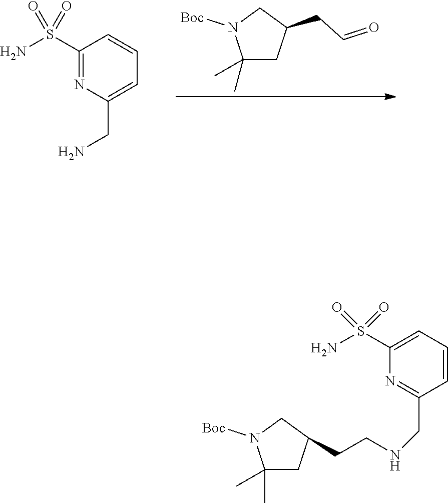 [SMILES: NCc1cccc(S(N)(=O)=O)n1 ; CC(C)(C)OC(=O)N1C[C@@H](CC=O)CC1(C)C ; CC(C)(C)OC(=O)N1C[C@@H](CCNCc2cccc(S(N)(=O)=O)n2)CC1(C)C]

Sodium triacetoxyborohydride (2.75 g, 12.586 mmol) was added to a solution of tert-butyl (4R)-2,2-dimethyl-4-(2-oxoethyl)pyrrolidine-1-carboxylate (1.5 g, 6.2156 mmol) and 6-(aminomethyl)pyridine-2-sulfonamide (hydrochloride salt) (1.696 g, 7.2638 mmol) in a mixture of dichloromethane (30 mL) and methanol (15 mL). The reaction mixture stirred at room temperature for 2 h. MeOH (20 mL) was added to the reaction mixture and it was evaporated in vacuo. The residue was treated with aqueous saturated NaHCO₃ solution (25 mL), water (25 mL) and EtOAc (50 mL). The layers were separated. The aqueous layer was further extracted with EtOAc (3×50 mL). The combined organic extracts were washed with brine (50 mL), dried over anhydrous sodium sulfate, filtered and concentrated in vacuo. The crude product was purified by silica gel chromatography, eluting with a 0% to 20% gradient of MeOH in DCM to give a white solid, tert-butyl (4S)-2,2-dimethyl-4-[2-[(6-sulfamoyl-2-pyridyl)methylamino]ethyl]pyrrolidine-1-carboxylate (1.43 g, 56%); ESI-MS m/z calc. 412.21442, found 413.2 (M+1)⁺; Retention time: 1.35 minutes (LC method E).

### Step 15: tert-Butyl (4S)-4-[2-[[6-[[2-chloro-6-[3-(2-dispiro[2.0.24.13]heptan-7-ylethoxy)pyrazol-1-yl]pyridine-3-carbonyl]sulfamoyl]-2-pyridyl]methylamino]ethyl]-2,2-dimethyl-pyrrolidine-1-carboxylate

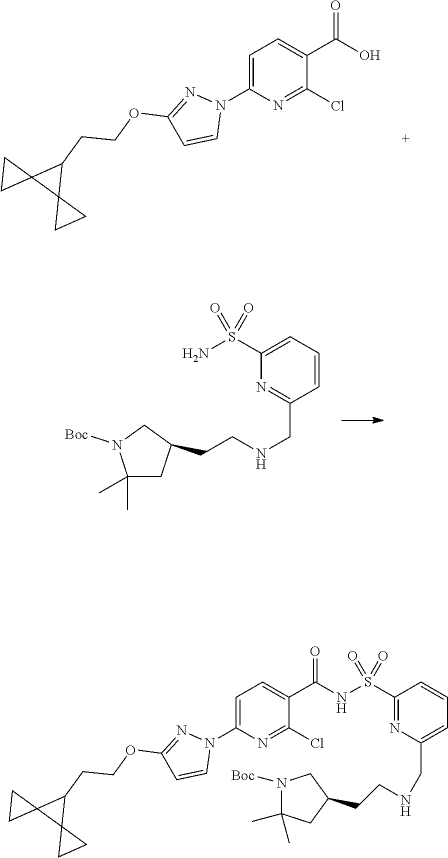 [SMILES: O=C(O)c1ccc(-n2ccc(OCCC3C4(CC4)C34CC4)n2)nc1Cl ; CC(C)(C)OC(=O)N1C[C@@H](CCNCc2cccc(S(N)(=O)=O)n2)CC1(C)C ; CC(C)(C)OC(=O)N1C[C@@H](CCNCc2cccc(S(=O)(=O)NC(=O)c3ccc(-n4ccc(OCCC5C6(CC6)C56CC6)n4)nc3Cl)n2)CC1(C)C]

To a solution of 2-chloro-6-[3-(2-dispiro[2.0.24.13]heptan-7-ylethoxy)pyrazol-1-yl]pyridine-3-carboxylic acid (315 mg, 0.8755 mmol) in THF (3 mL) was added 1,1′-carbonyldiimidazole (250 mg, 1.5418 mmol) and the mixture was stirred at room temperature for 2 h. In a separate flask, a solution of tert-butyl (4S)-2,2-dimethyl-4-[2-[(6-sulfamoyl-2-pyridyl)methylamino]ethyl]pyrrolidine-1-carboxylate (300 mg, 0.7272 mmol) in THF (3 mL) was prepared and it was subsequently added into the activated acid solution. 1,8-Diazabicyclo[5.4.0]undec-7-ene (335.94 mg, 0.33 mL, 2.2067 mmol) was added and the reaction mixture was stirred at room temperature for 18 h. After, the solvents were removed under reduced pressure. The crude was purified reverse phase chromatography using a C₁₈ column and eluting with a gradient of MeCN in water (containing 0.1% of formic acid), and then by silica gel chromatography eluting with a gradient of MeOH in dichloromethane (0 to 20%). This gave a yellow solid, tert-butyl (4S)-4-[2-[[6-[[2-chloro-6-[3-(2-dispiro[2.0.24.13]heptan-7-ylethoxy)pyrazol-1-yl]pyridine-3-carbonyl]sulfamoyl]-2-pyridyl]methylamino]ethyl]-2,2-dimethyl-pyrrolidine-1-carboxylate (353.3 mg, 42%); ESI-MS m/z calc. 753.30756, found 754.2 (M+1)⁺; Retention time: 1.92 minutes (LC method E).

### Step 16: 2-Chloro-N-[[6-[[2-[(3S)-5,5-dimethylpyrrolidin-3-yl]ethylamino]methyl]-2-pyridyl]sulfonyl]-6-[3-(2-dispiro[2.0.24.13]heptan-7-ylethoxy)pyrazol-1-yl]pyridine-3-carboxamide

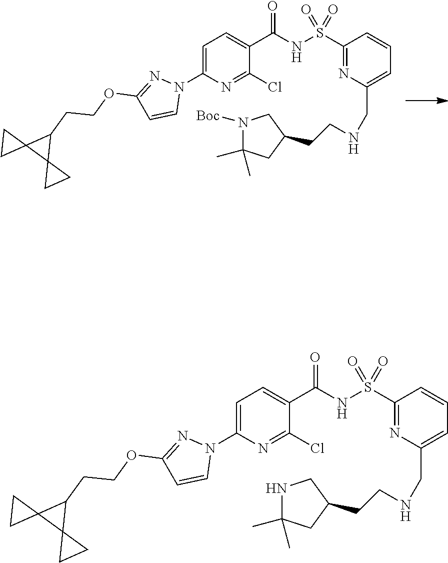 [SMILES: CC(C)(C)OC(=O)N1C[C@@H](CCNCc2cccc(S(=O)(=O)NC(=O)c3ccc(-n4ccc(OCCC5C6(CC6)C56CC6)n4)nc3Cl)n2)CC1(C)C ; CC1(C)C[C@H](CCNCc2cccc(S(=O)(=O)NC(=O)c3ccc(-n4ccc(OCCC5C6(CC6)C56CC6)n4)nc3Cl)n2)CN1]

To a solution of tert-butyl (4S)-4-[2-[[6-[[2-chloro-6-[3-(2-dispiro[2.0.24.13]heptan-7-ylethoxy)pyrazol-1-yl]pyridine-3-carbonyl]sulfamoyl]-2-pyridyl]methylamino]ethyl]-2,2-dimethyl-pyrrolidine-1-carboxylate (350 mg, 0.3011 mmol) in dichloromethane (7 mL) at room temperature was added 4 M HCl in dioxane (1.25 mL, 5.00 mmol) and the mixture was stirred for 2 h. Solvent was then evaporated and the crude mixture was purified by reverse phase chromatography, eluting with a gradient of MeOH in water (containing 0.1% of formic acid). The solvents were removed in vacuo and an aqueous solution of 1 N NaOH (50 mL) was added. The mixture was extracted with EtOAc (3×100 mL), washed with brine (100 mL), dried over anhydrous sodium sulfate, filtered and concentrated in vacuo to afford 2-chloro-N-[[6-[[2-[(3S)-5,5-dimethylpyrrolidin-3-yl]ethylamino]methyl]-2-pyridyl]sulfonyl]-6-[3-(2-dispiro[2.0.24.13]heptan-7-ylethoxy)pyrazol-1-yl]pyridine-3-carboxamide (162.8 mg, 83%) as a white solid. ¹H NMR (400 MHz, DMSO-d₆) δ 8.33 (d, J=2.9 Hz, 1H), 8.14 (d, J=8.3 Hz, 1H), 7.89-7.83 (m, 1H), 7.76 (d, J=6.8 Hz, 1H), 7.61 (d, J=8.3 Hz, 1H), 7.45 (d, J=7.6 Hz, 1H), 6.08 (d, J=2.7 Hz, 1H), 4.22 (t, J=6.6 Hz, 2H), 3.80 (s, 2H), 3.35 (br s, 2H), 3.19-3.08 (m, 1H), 2.66-2.59 (m, 1H), 2.35-2.21 (m, 1H), 1.85-1.76 (m, 3H), 1.54-1.43 (m, 3H), 1.29-1.14 (m, 6H), 1.11 (s, 3H), 0.88-0.78 (m, 4H), 0.67-0.61 (m, 2H), 0.53-0.47 (m, 2H). [Note: 1H is missing from the overall count of 40 protons from the product (C₃₂H₄₀ClN₇O₄S).] ESI-MS m/z calc. 653.2551, found 654.2 (M+1)⁺; Retention time: 1.49 minutes (LC method E).

### Step 17: (14S)-8-[3-(2-{Dispiro[2.0.24.13]heptan-7-yl}ethoxy)-1H-pyrazol-1-yl]-12,12-dimethyl-2λ⁶-thia-3,9,11,17,23-pentaazatetracyclo[17.3.1.111,14.05,10]tetracosa-1(23),5,7,9,19,21-hexaene-2,2,4-trione (Compound 433)

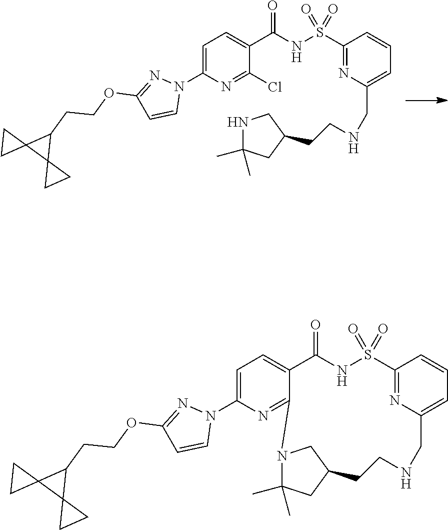 [SMILES: CC1(C)C[C@H](CCNCc2cccc(S(=O)(=O)NC(=O)c3ccc(-n4ccc(OCCC5C6(CC6)C56CC6)n4)nc3Cl)n2)CN1 ; CC1(C)C[C@@H]2CCNCc3cccc(n3)S(=O)(=O)NC(=O)c3ccc(-n4ccc(OCCC5C6(CC6)C56CC6)n4)nc3N1C2]

To a solution of 2-chloro-N-[[6-[[2-[(3S)-5,5-dimethylpyrrolidin-3-yl]ethylamino]methyl]-2-pyridyl]sulfonyl]-6-[3-(2-dispiro[2.0.24.13]heptan-7-ylethoxy)pyrazol-1-yl]pyridine-3-carboxamide (150 mg, 0.2293 mmol) in DMSO (4 mL) was added potassium carbonate (250 mg, 1.8089 mmol) and the mixture was stirred 24 h at 120° C. The mixture was then cooled to room temperature and was directly purified by reverse phase chromatography eluting with a gradient of MeCN in water (containing 0.1% of formic acid) (5% to 100%). This gave (34.2 mg) of product, mixed with dimeric side products. The resulting mixture was then re-purified by reverse phase chromatography (0% to 95% H₂O:CH₃CN with 0.1% HCO₂H modifier). After evaporation, 1N NaOH (10 mL) was added and the mixture was extracted with EtOAc (3×20 mL). This gave (14S)-8-[3-(2-{dispiro[2.0.24.13]heptan-7-yl}ethoxy)-1H-pyrazol-1-yl]-12,12-dimethyl-2λ⁶-thia-3,9,11,17,23-pentaazatetracyclo[17.3.1.111,14.05,10]tetracosa-1(23),5,7,9,19,21-hexaene-2,2,4-trione (8.6 mg, 3%) as a white solid. ¹H NMR (400 MHz, DMSO-d₆) δ 8.15 (d, J=2.7 Hz, 1H), 7.92-7.81 (m, 2H), 7.53 (d, J=8.3 Hz, 1H), 7.45 (d, J=7.3 Hz, 1H), 6.76 (d, J=8.1 Hz, 1H), 5.99 (d, J=2.7 Hz, 1H), 4.19 (t, J=6.7 Hz, 2H), 3.96-3.83 (m, 2H), 3.30-3.28 (m, 1H), 3.22-3.12 (m, 1H), 2.46-2.41 (m, 1H), 2.29-2.17 (m, 1H), 2.15-2.04 (m, 1H), 1.87-1.78 (m, 3H), 1.72-1.64 (m, 1H), 1.63-1.43 (m, 10H), 0.85-0.82 (m, 4H), 0.67-0.62 (m, 2H), 0.53-0.46 (m, 2H). [Note: 1H is missing from the overall count of 39 protons from the product (C₃₂H₃₉N₇O₄S).] ESI-MS 111/Z calc. 617.27844, found 618.3 (M+1)⁺; Retention time: 3.41 minutes (LC Method F).

### Example 165: Preparation of (14S)-16-(2,2-Dimethylpropyl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 434) (14S)-16-(2,2-Dimethylpropyl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 435) and (14S)-16-(2,2-dimethylpropyl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 436)

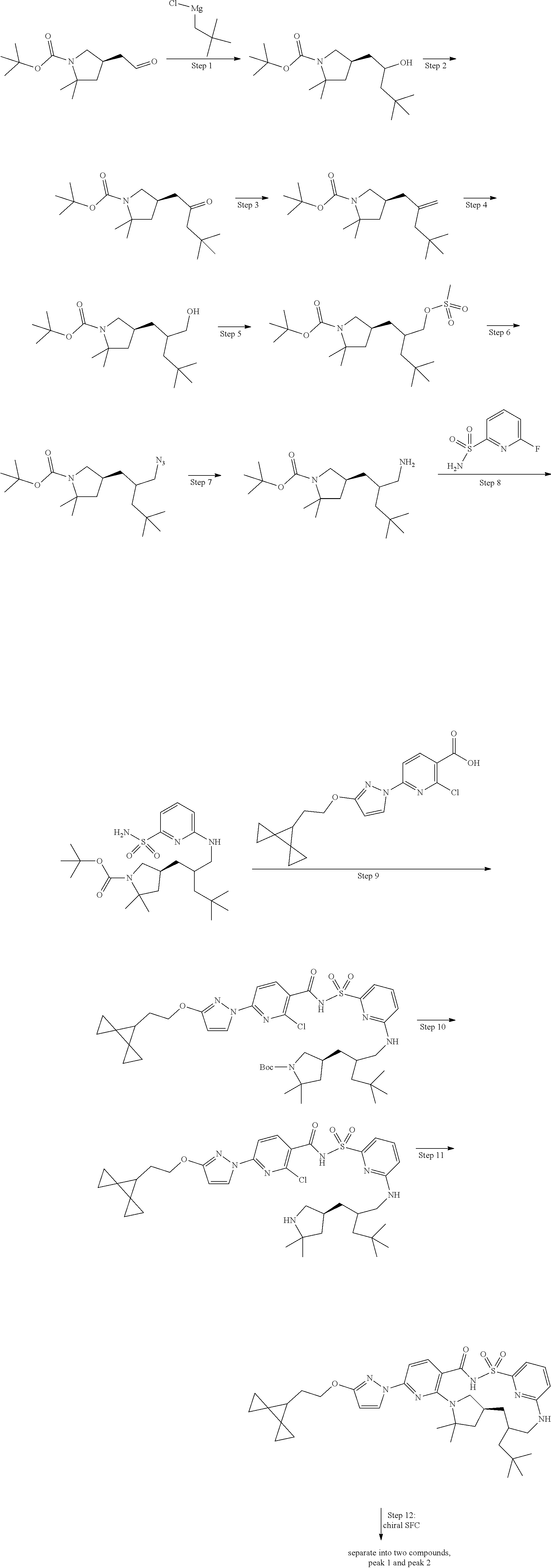

### Step 1: tert-Butyl (4R)-4-(2-hydroxy-4,4-dimethyl-pentyl)-2,2-dimethyl-pyrrolidine-1-carboxylate

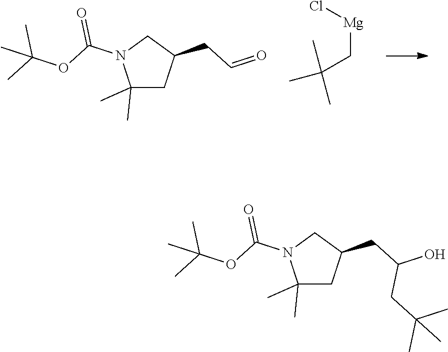

To a solution of tert-butyl (4R)-2,2-dimethyl-4-(2-oxoethyl)pyrrolidine-1-carboxylate (2 g, 7.5085 mmol) in THF (32 mL) at 0° C. was added dropwise neopentylmagnesium chloride solution in THF (16.7 mL of 1 M, 16.700 mmol). The mixture was stirred at 0° C. for 1 h then quenched with saturated aqueous ammonium chloride solution (25 mL). The mixture was diluted in ethyl acetate (100 mL) and the phases were separated. The aqueous phase was extracted with ethyl acetate (2×50 mL). The combined organic layers were dried over anhydrous sodium sulfate, filtered and concentrated under reduced pressure. The crude residue was purified by silica gel chromatography eluting from 0% to 50% ethyl acetate in heptanes to afford tert-butyl (4R)-4-(2-hydroxy-4,4-dimethyl-pentyl)-2,2-dimethyl-pyrrolidine-1-carboxylate (1.44 g, 61%) as a white solid. ESI-MS m/z calc. 313.2617, found 258.2 (M-tBu+2H=M−55)⁺; Retention time: 2.06 minutes (LC method E).

### Step 2: tert-Butyl (4R)-4-(4,4-dimethyl-2-oxo-pentyl)-2,2-dimethyl-pyrrolidine-1-carboxylate

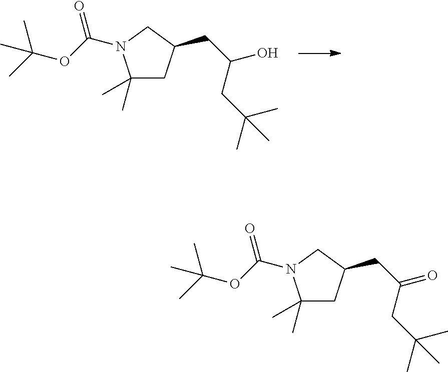

To a solution of tert-butyl (4R)-4-(2-hydroxy-4,4-dimethyl-pentyl)-2,2-dimethyl-pyrrolidine-1-carboxylate (1.44 g, 4.5891 mmol) in dichloromethane (15 mL) was added Dess-Martin periodinane (2.29 g, 5.3991 mmol) and the mixture was stirred at room temperature for 30 min. The mixture was quenched with saturated aqueous sodium bicarbonate solution (40 mL) and extracted with dichloromethane (2×40 mL). The organic phase was washed with saturated aqueous sodium bicarbonate solution (20 mL) and brine (20 mL), dried over sodium sulfate, filtered and concentrated to dryness. The crude material was purified by silica gel chromatography with 0 to 40% ethyl acetate in heptane to give tert-butyl (4R)-4-(4,4-dimethyl-2-oxo-pentyl)-2,2-dimethyl-pyrrolidine-1-carboxylate (920 mg, 61%) as a white solid. ESI-MS m/z calc. 311.24603, found 334.2 (M+Na=M+23)⁺; Retention time: 2.11 minutes (LC method E).

### Step 3: tert-Butyl (4S)-4-(4,4-dimethyl-2-methylene-pentyl)-2,2-dimethyl-pyrrolidine-1-carboxylate

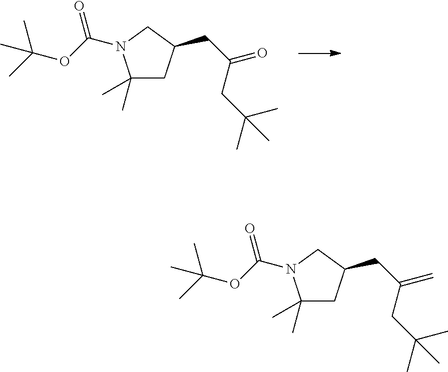

To a solution of methyl(triphenyl)phosphonium bromide (1.64 g, 4.5910 mmol) in THF (8.5 mL) under nitrogen at 0° C. was added dropwise sodium bis(trimethylsilyl)amide in THF (4.6 mL of 1 M, 4.6000 mmol) and the mixture was stirred at room temperature for 1 h. The resulting yellow solution was treated with tert-butyl (4R)-4-(4,4-dimethyl-2-oxo-pentyl)-2,2-dimethyl-pyrrolidine-1-carboxylate (580 mg, 1.8268 mmol) in THF (8.5 mL) and stirred at 70° C. for 16 h. Upon cooling to room temperature, water (30 mL) was added and the mixture was extracted with ethyl acetate (3×30 mL). The combined organic phase was washed with brine (2×20 mL), dried over sodium sulfate, filtered and concentrated to dryness. The crude residue was purified by silica gel chromatography on silica gel eluting with 0% to 20% ethyl acetate in heptanes to give tert-butyl (4S)-4-(4,4-dimethyl-2-methylene-pentyl)-2,2-dimethyl-pyrrolidine-1-carboxylate (444 mg, 77%) as a colorless oil. ESI-MS m/z calc. 309.26678, found 254.2 (M-tBu+2H=M−55)⁺; Retention time: 2.51 minutes (LC method E).

### Step 4: tert-Butyl (4S)-4-[2-(hydroxymethyl)-4,4-dimethyl-pentyl]-2,2-dimethyl-pyrrolidine-1-carboxylate

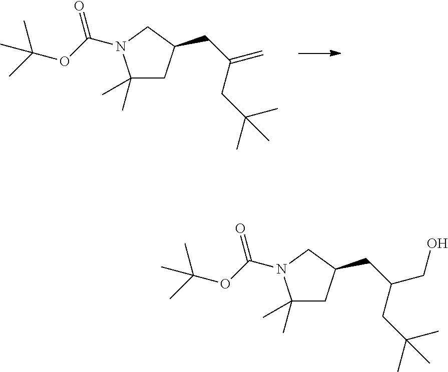

A solution of tert-butyl (4S)-4-(4,4-dimethyl-2-methylene-pentyl)-2,2-dimethyl-pyrrolidine-1-carboxylate (444 mg, 1.3629 mmol) in THF (15 mL) was cooled to 0° C. and 9-borabicyclo[3.3.1]nonane solution in THF (9.5 mL of 0.5 M, 4.7500 mmol) was added. The resulting mixture was stirred for 16 h. 1 N NaOH (6.9 mL, 6.9 mmol) and hydrogen peroxide (666 mg, 0.6000 mL, 6.8529 mmol) were slowly added at 0° C. and the mixture was stirred for 24 h. The mixture was diluted with water (30 mL) and extracted with ethyl acetate (3×40 mL). The organic phase was dried over sodium sulfate, filtered and concentrated to dryness. The crude oil was purified by silica gel chromatography using a gradient of 0% to 20% ethyl acetate in heptanes to give tert-butyl (4S)-4-[2-(hydroxymethyl)-4,4-dimethyl-pentyl]-2,2-dimethyl-pyrrolidine-1-carboxylate (440 mg, 89%) as a colorless oil. ESI-MS m/z calc. 327.27734, found 272.2 (M-tBu+2H=M−55)⁺; Retention time: 2.08 minutes (LC method E).

### Step 5: tert-Butyl (4S)-4-[4,4-dimethyl-2-(methylsulfonyloxymethyl)pentyl]-2,2-dimethyl-pyrrolidine-1-carboxylate

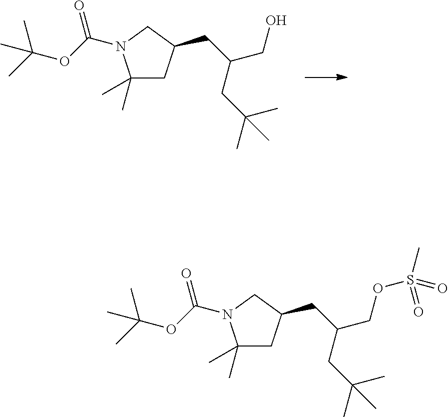

To a 0° C. solution of tert-butyl (4S)-4-[2-(hydroxymethyl)-4,4-dimethyl-pentyl]-2,2-dimethyl-pyrrolidine-1-carboxylate (440 mg, 1.2092 mmol) in DCM (6.5 mL) was added triethylamine (159.72 mg, 0.22 mL, 1.5784 mmol) and then methanesulfonyl chloride (165.76 mg, 0.112 mL, 1.4470 mmol). The ice bath was removed and the mixture was stirred for 2.5 h. The mixture was partitioned between dichloromethane (25 mL) and water (20 mL). The phases were separated and the aqueous layer was extracted with dichloromethane (2×20 mL). The organic phase was washed with brine (2×20 mL), dried over sodium sulfate, filtered and concentrated to dryness to afford crude tert-butyl (4S)-4-[4,4-dimethyl-2-(methylsulfonyloxymethyl)pentyl]-2,2-dimethyl-pyrrolidine-1-carboxylate (540 mg, 101%) as a colorless oil. ESI-MS m/z calc. 405.25488, found 350.2 (M-tBu+2H=M−55)⁺; Retention time: 2.09 minutes (LC method E).

### Step 6: tert-Butyl (4S)-4-[2-(azidomethyl)-4,4-dimethyl-pentyl]-2,2-dimethyl-pyrrolidine-1-carboxylate

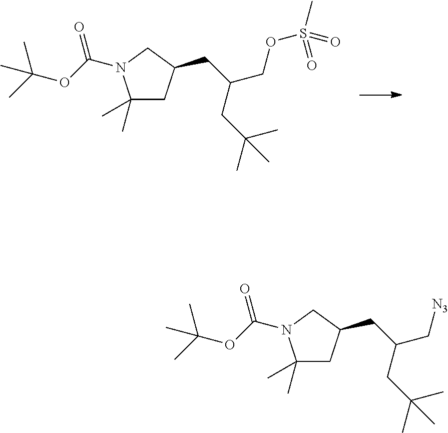 [SMILES: CC(C)(C)CC(COS(C)(=O)=O)C[C@@H]1CN(C(=O)OC(C)(C)C)C(C)(C)C1 ; CC(C)(C)CC(CN=[N+]=[N-])C[C@@H]1CN(C(=O)OC(C)(C)C)C(C)(C)C1]

To a solution of tert-butyl (4S)-4-[4,4-dimethyl-2-(methylsulfonyloxymethyl)pentyl]-2,2-dimethyl-pyrrolidine-1-carboxylate (540 mg, 1.2169 mmol) in DMF (8.5 mL) was added sodium azide (175 mg, 2.6919 mmol) and the mixture was stirred for 16 h at 65° C. The mixture was diluted with water (25 mL) and extracted with ethyl acetate (2×25 mL). The organic phase was washed with brine (3×25 mL), dried over sodium sulfate, filtered and concentrated to dryness to give crude tert-butyl (4S)-4-[2-(azidomethyl)-4,4-dimethyl-pentyl]-2,2-dimethyl-pyrrolidine-1-carboxylate (446 mg, 95%) as a colorless oil. ESI-MS m/z calc. 352.2838, found 253.4 (M-Boc+2H=M−99)⁺; Retention time: 2.41 minutes (LC method E).

### Step 7: tert-Butyl (4S)-4-[2-(aminomethyl)-4,4-dimethyl-pentyl]-2,2-dimethyl-pyrrolidine-1-carboxylate

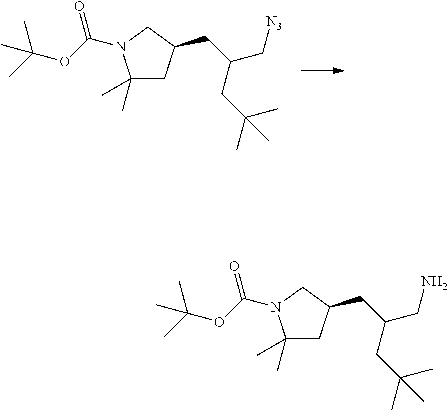

A solution of tert-butyl (4S)-4-[2-(azidomethyl)-4,4-dimethyl-pentyl]-2,2-dimethyl-pyrrolidine-1-carboxylate (446 mg, 1.1513 mmol) in MeOH (11 mL) was degassed by bubbling nitrogen for 10 min. To the solution was added 10% palladium on carbon (50% wet, 230 mg, 0.1081 mmol) and the mixture was degassed with nitrogen for additional 10 min. Hydrogen was bubbled to the mixture for 10 min and then the mixture was stirred at room temperature under hydrogen atmosphere for 4 h. The mixture was filtered through Celite, and washed with methanol (25 mL). The filtrate was concentrated under reduced pressure to give crude tert-butyl (4S)-4-[2-(aminomethyl)-4,4-dimethyl-pentyl]-2,2-dimethyl-pyrrolidine-1-carboxylate (349 mg, 88%) as a colorless oil. ¹H NMR (400 MHz, CDCl₃) δ 3.78-3.59 (m, 1H), 2.98-2.82 (m, 1H), 2.66-2.54 (m, 1H), 2.21-2.12 (m, 1H), 1.95-1.88 (m, 1H), 1.75-1.66 (m, 2H), 1.53-1.34 (m, 16H), 1.32-1.22 (m, 6H), 0.91-0.88 (m, 9H). ESI-MS m/z calc. 326.29333, found 327.4 (M+1)⁺; Retention time: 1.51 minutes (LC method E).

### Step 8: tert-Butyl (4S)-4-[4,4-dimethyl-2-[[(6-sulfamoyl-2-pyridyl)amino]methyl]pentyl]-2,2-dimethyl-pyrrolidine-1-carboxylate

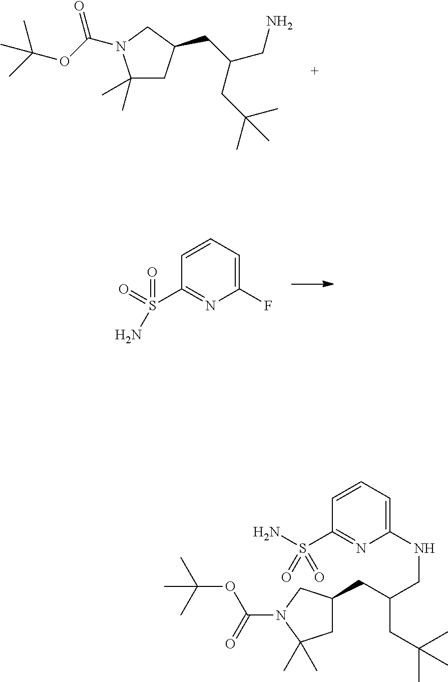

To a solution of tert-butyl (4S)-4-[2-(aminomethyl)-4,4-dimethyl-pentyl]-2,2-dimethyl-pyrrolidine-1-carboxylate (349 mg, 1.0079 mmol) in MeCN (4.25 mL) was added DIPEA (556.50 mg, 0.75 mL, 4.3058 mmol) and 6-fluoropyridine-2-sulfonamide (1.9 g, 10.785 mmol) under inert atmosphere. The vial was sealed and the mixture was heated at 120° C. for 18 h. The resulting mixture was directly purified by reverse phase chromatography eluting with 5% to 100% methanol in water to give tert-butyl (4S)-4-[4,4-dimethyl-2-[[(6-sulfamoyl-2-pyridyl)amino]methyl]pentyl]-2,2-dimethyl-pyrrolidine-1-carboxylate (295 mg, 61%) as a white solid. ESI-MS m/z calc. 482.29266, found 427.2 (M-tBu+2H=M−55)⁺; Retention time: 4.8 minutes (LC Method F).

### Step 9: tert-Butyl (4S)-4-[2-[[[6-[[2-chloro-6-[3-(2-dispiro[2.0.24.13]heptan-7-ylethoxy)pyrazol-1-yl]pyridine-3-carbonyl]sulfamoyl]-2-pyridyl]amino]methyl]-4,4-dimethyl-pentyl]-2,2-dimethyl-pyrrolidine-1-carboxylate

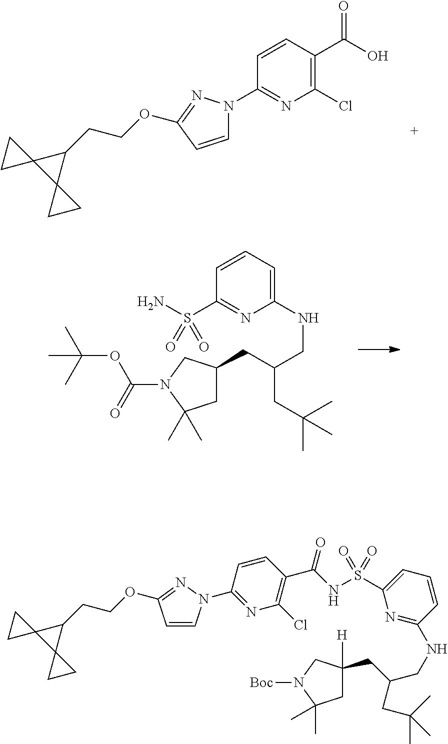 [SMILES: O=C(O)c1ccc(-n2ccc(OCCC3C4(CC4)C34CC4)n2)nc1Cl ; CC(C)(C)CC(CNc1cccc(S(N)(=O)=O)n1)C[C@@H]1CN(C(=O)OC(C)(C)C)C(C)(C)C1 ; CC(C)(C)CC(CNc1cccc(S(=O)(=O)NC(=O)c2ccc(-n3ccc(OCCC4C5(CC5)C45CC5)n3)nc2Cl)n1)C[C@@H]1CN(C(=O)OC(C)(C)C)C(C)(C)C1]

A round-bottomed flask was charged with 2-chloro-6-[3-(2-dispiro[2.0.24.13]heptan-7-ylethoxy)pyrazol-1-yl]pyridine-3-carboxylic acid (130 mg, 0.3035 mmol) and THF (1.8 mL). Carbonyldiimidazole (49 mg, 0.3022 mmol) was added and the mixture was stirred under nitrogen at room temperature for 2 h. In a separate flask, a solution of tert-butyl (4S)-4-[4,4-dimethyl-2-[[(6-sulfamoyl-2-pyridyl)amino]methyl]pentyl]-2,2-dimethyl-pyrrolidine-1-carboxylate (90 mg, 0.1865 mmol) in THF (1.2 mL) was prepared and it was added via syringe into the activated acid solution. 1,8-Diazabicyclo[5.4.0]undec-7-ene (1.7306 g, 1.7 mL, 11.368 mmol) was added and the reaction mixture was stirred at room temperature under nitrogen for 3 h. After concentration in vacuo, the crude material was purified by reverse phase chromatography eluting with a gradient of 5% to 100% of methanol in water to give tert-butyl (4S)-4-[2-[[[6-[[2-chloro-6-[3-(2-dispiro[2.0.24.13]heptan-7-ylethoxy)pyrazol-1-yl]pyridine-3-carbonyl]sulfamoyl]-2-pyridyl]amino]methyl]-4,4-dimethyl-pentyl]-2,2-dimethyl-pyrrolidine-1-carboxylate (142 mg, 88%) as a white solid. ¹H NMR (400 MHz, CDCl₃) δ 12.68-11.96 (m, 1H), 8.36-8.27 (m, 1H), 7.99-7.91 (m, 1H), 7.72-7.52 (m, 3H), 6.54-6.50 (m, 1H), 5.95-5.91 (m, 1H), 4.97-4.87 (m, 1H), 4.38-4.21 (m, 3H), 4.09-3.95 (m, 1H), 3.73-3.67 (m, 1H), 3.10-2.89 (m, 1H), 2.83-2.76 (m, 1H), 2.50-2.41 (m, 1H), 2.25-2.17 (m, 1H), 1.92-1.77 (m, 3H), 1.69-1.61 (m, 1H), 1.54-1.41 (m, 4H), 1.36-1.22 (m, 14H), 1.17-0.97 (m, 2H), 0.84-0.82 (m, 9H), 0.77-0.75 (m, 2H), 0.67-0.64 (m, 2H), 0.52-0.49 (m, 2H). [Note: 1H is missing from the overall count of 58 protons from the product (C₄₂H₅₈ClN₇O₆S).]

### Step 10: 2-Chloro-N-[[6-[[2-[[(3S)-5,5-dimethylpyrrolidin-3-yl]methyl]-4,4-dimethyl-pentyl]amino]-2-pyridyl]sulfonyl]-6-[3-(2-dispiro[2.0.24.13]heptan-7-ylethoxy)pyrazol-1-yl]pyridine-3-carboxamide

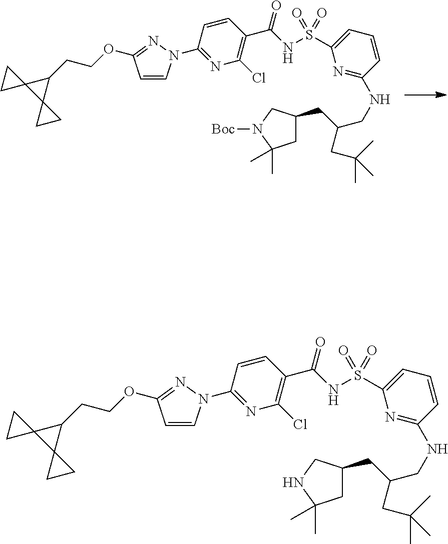 [SMILES: CC(C)(C)CC(CNc1cccc(S(=O)(=O)NC(=O)c2ccc(-n3ccc(OCCC4C5(CC5)C45CC5)n3)nc2Cl)n1)C[C@@H]1CN(C(=O)OC(C)(C)C)C(C)(C)C1 ; CC(C)(C)CC(CNc1cccc(S(=O)(=O)NC(=O)c2ccc(-n3ccc(OCCC4C5(CC5)C45CC5)n3)nc2Cl)n1)C[C@@H]1CNC(C)(C)C1]

To a solution of tert-butyl (4S)-4-[2-[[[6-[[2-chloro-6-[3-(2-dispiro[2.0.24.13]heptan-7-ylethoxy)pyrazol-1-yl]pyridine-3-carbonyl]sulfamoyl]-2-pyridyl]amino]methyl]-4,4-dimethyl-pentyl]-2,2-dimethyl-pyrrolidine-1-carboxylate (142 mg, 0.1648 mmol) in dichloromethane (1.6 mL) was added 4 M HCl solution in dioxane (0.5 mL, 2.0 mmol) and the mixture was stirred for 2 h. The mixture was concentrated under reduced pressure to give 2-chloro-N-[[6-[[2-[[(3S)-5,5-dimethylpyrrolidin-3-yl]methyl]-4,4-dimethyl-pentyl]amino]-2-pyridyl]sulfonyl]-6-[3-(2-dispiro[2.0.24.13]heptan-7-ylethoxy)pyrazol-1-yl]pyridine-3-carboxamide (hydrochloride salt) (135 mg, 103%) as a white solid. ¹H NMR (400 MHz, DMSO-d₆) δ 12.71 (br. s, 1H), 8.87-8.79 (m, 2H), 8.42-8.39 (m, 1H), 8.12-8.09 (m, 1H), 7.75-7.69 (m, 1H), 7.62 (t, J=7.8 Hz, 1H), 7.22 (br. s, 1H), 7.17 (d, J=7.3 Hz, 1H), 6.81-6.77 (m, 1H), 6.19-6.17 (m, 1H), 4.27-4.22 (m, 4H), 3.52-3.47 (m, 1H), 3.36-3.22 (m, 2H), 3.14-3.02 (m, 1H), 2.86-2.72 (m, 1H), 2.11-1.91 (m, 1H), 1.82 (q, J=6.3 Hz, 2H), 1.62-1.45 (m, 3H), 1.38-1.23 (m, 8H), 1.11-1.05 (m, 2H), 0.85-0.79 (m, 11H), 0.65-0.63 (m, 2H), 0.51-0.48 (m, 2H). ESI-MS m/z calc. 723.3334, found 724.4 (M+1)⁺; Retention time: 1.86 minutes (isomer 1) (LC method E). ESI-MS m/z calc. 723.3334, found 724.4 (M+1)⁺; Retention time: 1.91 minutes (isomer 2) (LC method E).

### Step 11: (14S)-16-(2,2-Dimethylpropyl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 434)

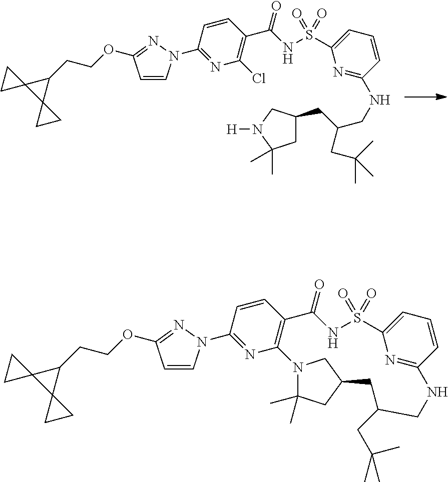 [SMILES: CC(C)(C)CC(CNc1cccc(S(=O)(=O)NC(=O)c2ccc(-n3ccc(OCCC4C5(CC5)C45CC5)n3)nc2Cl)n1)C[C@@H]1CNC(C)(C)C1 ; CC(C)(C)CC1CNc2cccc(n2)S(=O)(=O)NC(=O)c2ccc(-n3ccc(OCCC4C5(CC5)C45CC5)n3)nc2N2C[C@@H](C1)CC2(C)C]

To a solution of 2-chloro-N-[[6-[[2-[[(3S)-5,5-dimethylpyrrolidin-3-yl]methyl]-4,4-dimethyl-pentyl]amino]-2-pyridyl]sulfonyl]-6-[3-(2-dispiro[2.0.24.13]heptan-7-ylethoxy)pyrazol-1-yl]pyridine-3-carboxamide (hydrochloride salt) (132.4 mg, 0.1692 mmol) in DMSO (2.3 mL) was added potassium carbonate (185 mg, 1.3386 mmol). The reaction tube was sealed and heated at 120° C. for 20 h. The mixture was filtered and directly purified by reverse phase chromatography using a gradient of 5% to 100% of methanol in water to afford (14S)-16-(2,2-dimethylpropyl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (51.5 mg, 44%) as an off-white solid. ¹H NMR (400 MHz, DMSO-d₆) δ 8.15 (d, J=2.0 Hz, 1H), 7.70-7.54 (m, 1H), 7.43-7.31 (m, 1H), 6.93-6.81 (m, 1H), 6.74 (d, J=7.8 Hz, 1H), 6.65-6.52 (m, 1H), 6.45-6.31 (m, 1H), 6.02-5.90 (m, 1H), 4.19 (t, J=6.2 Hz, 2H), 3.96-3.81 (m, 1H), 3.57-3.46 (m, 1H), 3.07-2.93 (m, 1H), 2.78-2.65 (m, 1H), 2.35-2.05 (m, 2H), 1.92-1.71 (m, 4H), 1.62-1.42 (m, 8H), 1.38-1.12 (m, 4H), 0.96-0.79 (m, 12H), 0.70-0.57 (m, 2H), 0.55-0.42 (m, 2H) [Note: 1H is missing from the overall count of 49 protons from the product (C₃₇H₄₉N₇O₄S).] Major isomer: ESI-MS m/z calc. 687.3567, found 688.3 (M+1)⁺; Retention time: 4.53 minutes (low ionization) (LC method F). Minor isomer: ESI-MS m/z calc. 687.3567, found 688.3 (M+1)⁺; Retention time: 4.48 minutes (low ionization) (LC method F).

### Step 12: (14S)-16-(2,2-Dimethylpropyl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 435) and (145)-16-(2,2-dimethylpropyl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (Compound 436)

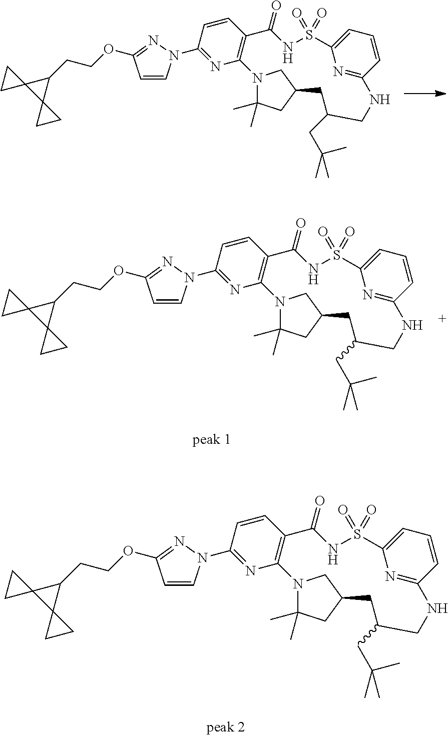 [SMILES: CC(C)(C)CC1CNc2cccc(n2)S(=O)(=O)NC(=O)c2ccc(-n3ccc(OCCC4C5(CC5)C45CC5)n3)nc2N2C[C@@H](C1)CC2(C)C ; CC(C)(C)CC1CNc2cccc(n2)S(=O)(=O)NC(=O)c2ccc(-n3ccc(OCCC4C5(CC5)C45CC5)n3)nc2N2C[C@@H](C1)CC2(C)C ; CC(C)(C)CC1CNc2cccc(n2)S(=O)(=O)NC(=O)c2ccc(-n3ccc(OCCC4C5(CC5)C45CC5)n3)nc2N2C[C@@H](C1)CC2(C)C]

(14S)-16-(2,2-dimethylpropyl)-8-[3-(2-dispiro[2.0.24.13]heptan-7-yl ethoxy)-1H-pyrazol-1-yl]-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (42.1 mg, 0.06120 mmol) (40:60 diastereomeric mixture) was subjected to chiral SFC using a ChiralPak IG column (250×10 mm; 5 μm) at 35° C. Mobile phase was 26% MeOH (with 20 mM NH₃), 74% CO₂ at a 10 mL/min flow. Concentration of the sample was 24 mg/mL in methanol (no modifier), injection volume 704 with an outlet pressure of 161 bar, detection wavelength of 210 nm. Evaporation of the solvents gave two isomers. The first isomer to elute was designated as Peak 1, and the second isomer to elute was designated as Peak 2.

Peak 1: (14S)-16-(2,2-dimethylpropyl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (10.7 mg, 64%). ¹H NMR (400 MHz, DMSO-d₆) δ 12.44 (s, 1H), 8.20 (d, J=2.7 Hz, 1H), 7.79 (d, J=8.0 Hz, 1H), 7.57 (s, 1H), 7.13 (s, 1H), 7.05 (s, 1H), 6.91 (d, J=8.2 Hz, 1H), 6.69 (d, J=8.4 Hz, 1H), 6.08 (s, 1H), 4.21 (t, J=6.7 Hz, 2H), 3.84-3.65 (m, 1H), 3.17-3.02 (m, 1H), 2.78-2.61 (overlapped with DMSO peak, m, 1H), 2.29 (s, 1H), 1.96-1.72 (m, 4H), 1.69-1.42 (m, 10H), 1.41-1.29 (m, 1H), 1.15-1.04 (m, 1H), 1.00-0.73 (m, 14H), 0.70-0.58 (m, 2H), 0.56-0.37 (m, 2H). ESI-MS m/z calc. 687.3567, found 688.4 (M+1)⁺; Retention time: 1.78 minutes (LC method G).

Peak 2: (14S)-16-(2,2-dimethylpropyl)-8-[3-(2-{dispiro[2.0.24.13]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5(10),6,8,19(23),20-hexaene-2,2,4-trione (19.7 mg, 70%); ¹H NMR (400 MHz, DMSO-d₆) δ 12.49 (s, 1H), 8.19 (d, J 2.7 Hz, 1H), 7.77 (s, 1H), 7.56 (s, 1H), 7.07 (s, 1H), 6.89 (broad s, 3H), 6.06 (s, 1H), 4.21 (t, J=6.6 Hz, 2H), 3.98 (br s, 1H), 3.41-3.38 (m, 1H), 2.80 (br s, 1H), 2.22 (br s, 1H), 1.86-1.73 (m, 4H), 1.65-1.42 (m, 10H), 1.39-1.35 (br m, 1H), 1.23-1.21 (m, 1H), 1.01-0.74 (m, 14H), 0.70-0.58 (m, 2H), 0.56-0.42 (m, 2H). ESI-MS m/z calc. 687.3567, found 688.4 (M+1)⁺; Retention time: 1.79 minutes (LC method G).

### Example 166: Bioactivity Assays for Compounds 1 to 397

Solutions

Base medium (ADF+++) consisted of Advanced DMEM/Ham's F12, 2 mM Glutamax, 10 mM HEPES, 1 μg/ml penicillin/streptomycin.

Intestinal enteroid maintenance medium (IEMM) consisted of ADF+++, 1×B27 supplement, 1×N2 supplement, 1.25 mM N-acetyl cysteine, 10 mM Nicotinamide, 50 ng/mL hEGF, 10 nM Gastrin, 1 μg/mL hR-spondin-1, 100 ng/mL hNoggin, TGF-b type 1 inhibitor A-83-01, 100 μg/mL Primocin, 10 μM P38 MAPK inhibitor SB202190.

Bath 1 Buffer consisted of 1 mM MgCl₂, 160 mM NaCl, 4.5 mM KCl, 10 mM HEPES, 10 mM Glucose, 2 mM CaCl₂).

Chloride Free Buffer consisted of 1 mM Magnesium Gluconate, 2 mM Calcium Gluconate, 4.5 mM Potassium Gluconate, 160 mM Sodium Gluconate, 10 mM HEPES, 10 mM Glucose.

Bath 1 Dye Solution consisted of Bath 1 Buffer, 0.04% Pluronic F127, 20 μM Methyl Oxonol, 30 μM CaCCinh-A01, 30 μM Chicago Sky Blue.

Chloride Free Dye Solution consisted of Chloride Free Buffer, 0.04% Pluronic F127, 20 μM Methyl Oxonol, 30 μM CaCCinh-A01, 30 μM Chicago Sky Blue.

Chloride Free Dye Stimulation Solution consisted of Chloride Free Dye Solution, 10 μM forskolin, 100 μM IBMX, and 300 nM Ivacaftor.

Cell Culture

Human intestinal epithelial enteroid cells were obtained from the Hubrecht Institute for Developmental Biology and Stem Cell Research, Utrecht, The Netherlands and expanded in T-Flasks as previously described (Dekkers J F, Wiegerinck C L, de Jonge H R, Bronsveld I, Janssens H M, de Winter-de Groot K M, Brandsma A M, de Jong N W M, Bijvelds M J C, Scholte B J, Nieuwenhuis E E S, van den Brink S, Clevers H, van der Ent C K, Middendorp S and M Beekman J M. A functional CFTR assay using primary cystic fibrosis intestinal organoids. Nat Med. 2013 July; 19(7):939-45).

Enteroid Cell Harvesting and Seeding

Cells were recovered in cell recovery solution, collected by centrifugation at 650 rpm for 5 min at 4° C., resuspended in TryPLE and incubated for 5 min at 37° C. Cells were then collected by centrifugation at 650 rpm for 5 min at 4° C. and resuspended in IEMM containing 10 μM ROCK inhibitor (RI). The cell suspension was passed through a 40 μm cell strainer and resuspended at 1×106 cells/mL in IEMM containing 10 μM RI. Cells were seeded at 5000 cells/well into multi-well plates and incubated for overnight at 37° C., 95% humidity and 5% CO₂ prior to assay.

Corrector Assay A—Membrane Potential Dye Assay

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 1 μM (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

### Corrector Assay B—Membrane Potertial Dye Assay

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 3 μM Compound A, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity). Compound A is:

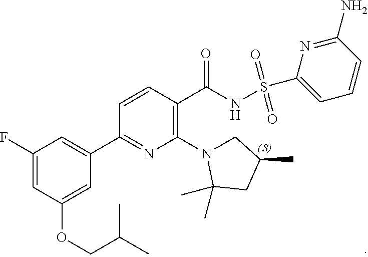

The following table represents CFTR modulating activity for representative compounds of the invention generated using one or more of the assays disclosed herein (EC₅₀: +++ is <1 μM; ++ is 1-3 μM; + is >3 μM; and ND is “not detected in this assay.” % Activity: +++ is >60%; ++ is 30-60%; + is <30%).

TABLE 3A
Bioactivity for Compounds 1-298

| | | Cor- | Cor- | Cor- | Cor- |
|---|---|---|---|---|---|
| | | rector | rector | rector | rector |
| Com- | | Assay | Assay | Assay | Assay |
| pound | | A EC₅₀ | A % | B EC₅₀ | B % |
| Number | Structure | (μM) | activity | (μM) | activity |
| 1 | 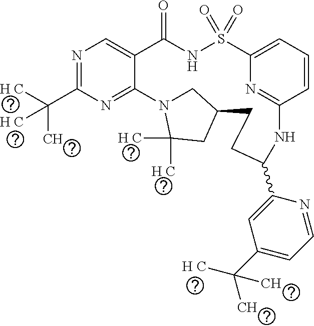 | ND | + | | |
| 2 | 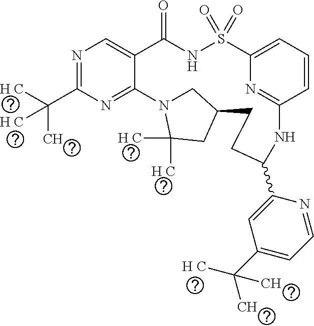 | +++ | +++ | | |
| 3 | 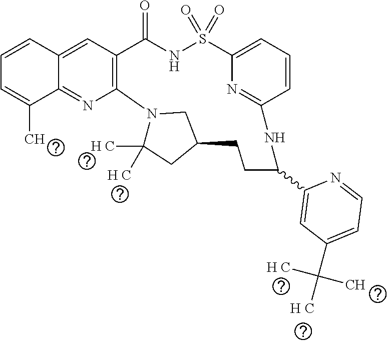 | +++ | +++ | | |
| 4 | 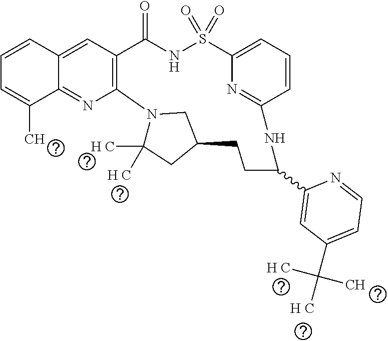 | +++ | ++ | | |
| 5 | 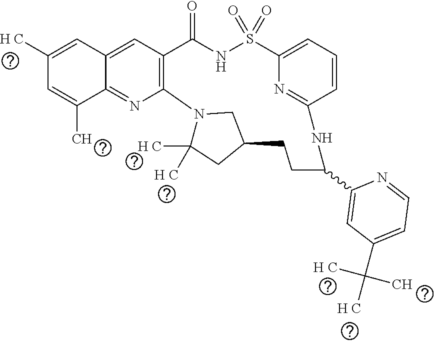 | +++ | +++ | | |
| 6 | 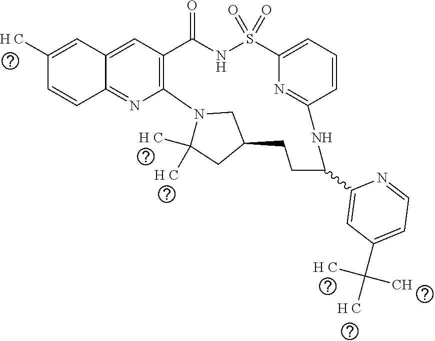 | +++ | ++ | | |
| 7 | 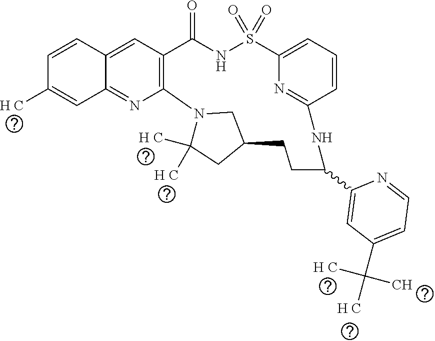 | +++ | +++ | | |
| 8 | 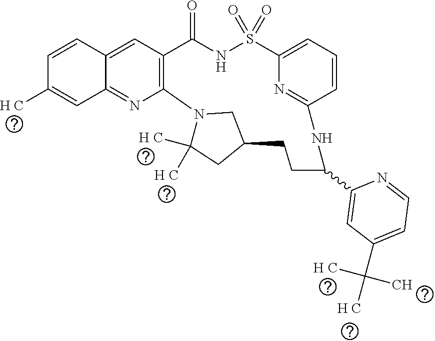 | +++ | ++ | | |
| 9 | 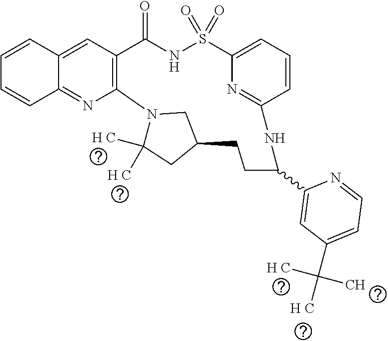 | +++ | +++ | | |
| 10 | 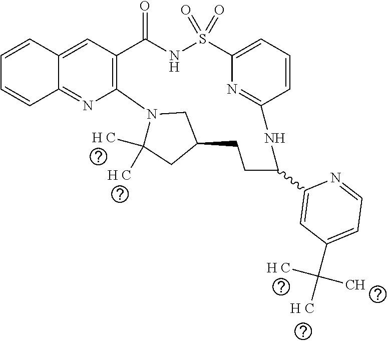 | +++ | ++ | | |
| 11 | 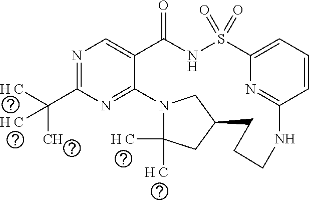 | ++ | ++ | | |
| 12 | 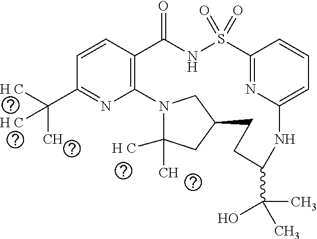 | +++ | +++ | | |
| 13 | 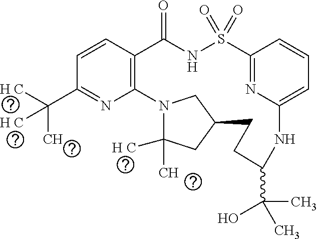 | +++ | ++ | | |
| 14 | 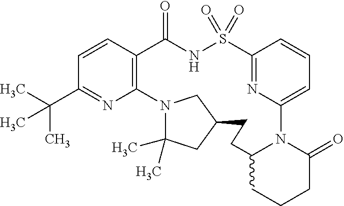 [SMILES: CC(C)(C)c1ccc2c(n1)N1C[C@@H](CCC3CCCC(=O)N3c3cccc(n3)S(=O)(=O)NC2=O)CC1(C)C] | +++ | ++ | | |
| 15 | 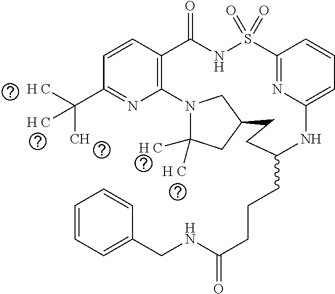 | +++ | +++ | | |
| 16 | 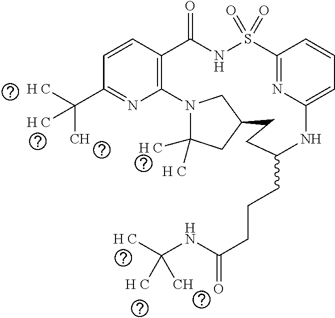 | +++ | +++ | | |
| 17 | 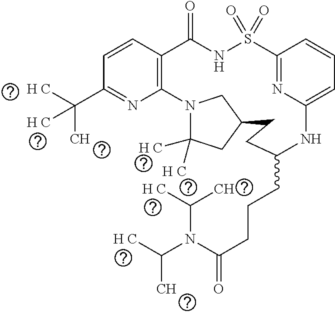 | +++ | +++ | | |
| 18 | 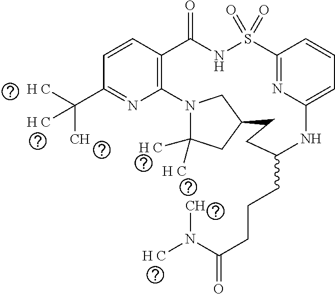 | +++ | +++ | | |
| 19 | 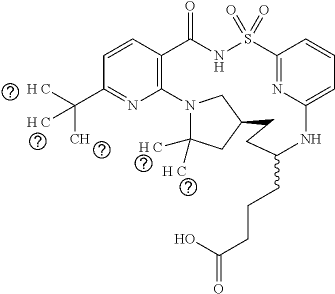 | ++ | +++ | | |
| 20 | 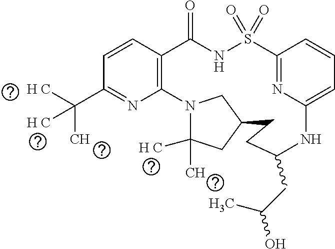 | +++ | ++ | | |
| 21 | 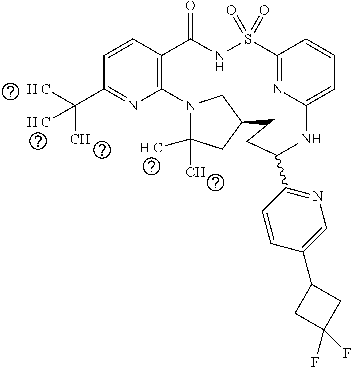 | +++ | +++ | | |
| 22 | 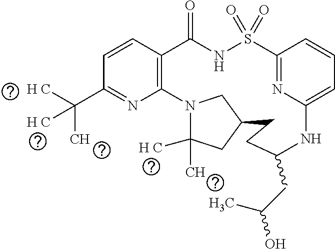 | +++ | ++ | | |
| 23 | 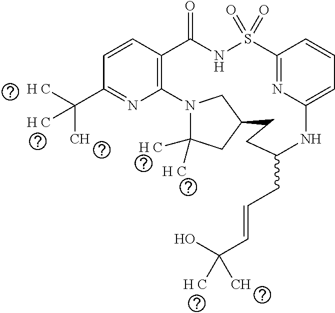 | +++ | +++ | | |
| 24 | 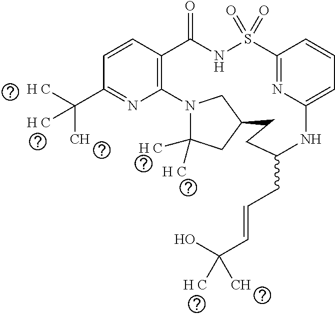 | +++ | ++ | | |
| 25 | 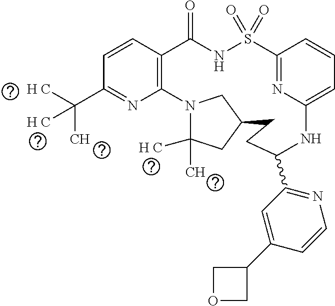 | +++ | +++ | | |
| 26 | 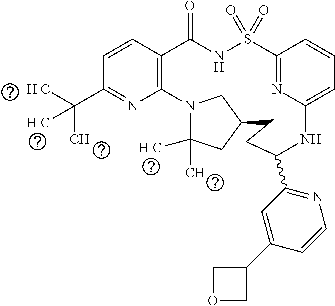 | +++ | +++ | | |
| 27 | 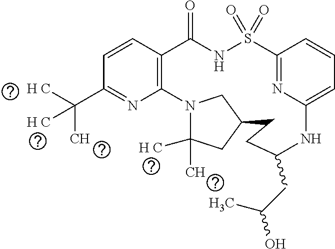 | +++ | ++ | | |
| 28 | 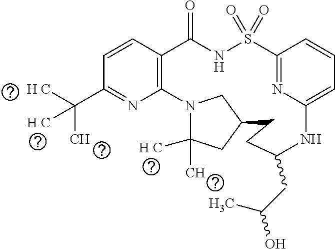 | +++ | +++ | | |
| 29 | 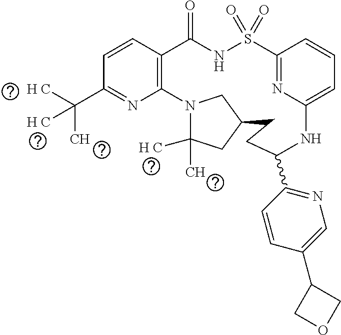 | +++ | +++ | | |
| 30 | 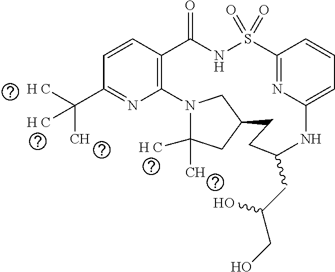 | +++ | ++ | | |
| 31 | 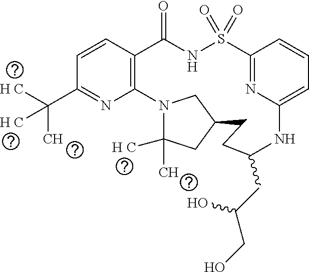 | +++ | ++ | | |
| 32 | 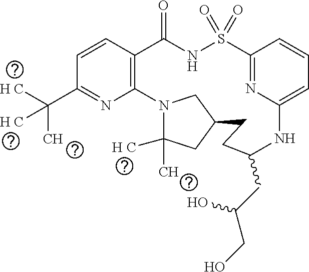 | +++ | +++ | | |
| 33 | 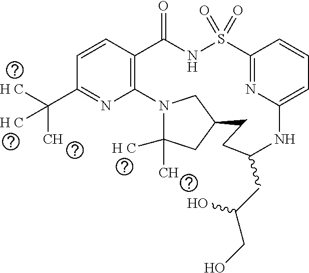 | +++ | +++ | | |
| 34 | 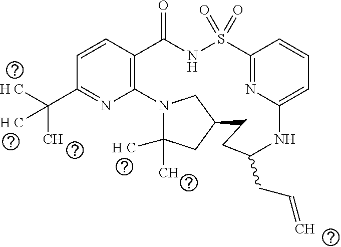 | +++ | +++ | | |
| 35 | 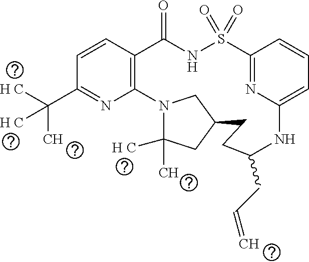 | +++ | ++ | | |
| 36 | 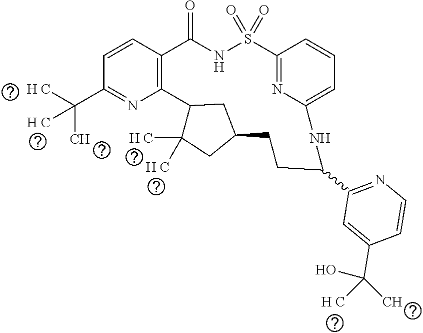 | +++ | +++ | | |
| 37 | 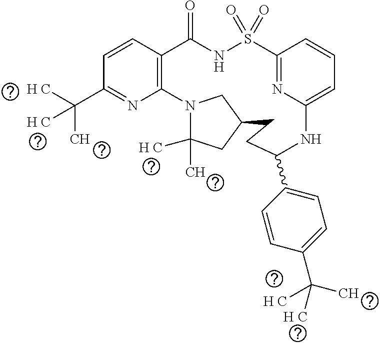 | +++ | ++ | | |
| 38 | 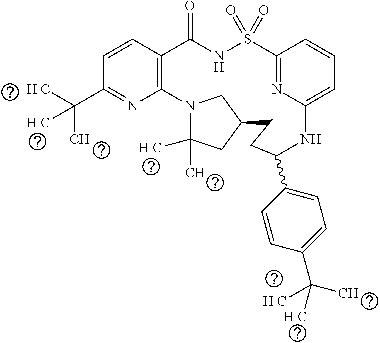 | +++ | +++ | | |
| 39 | 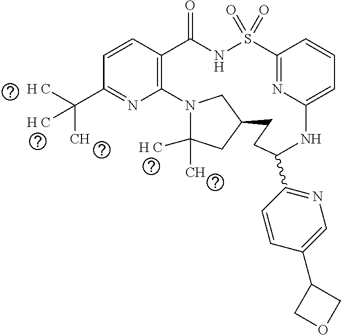 | +++ | +++ | | |
| 40 | 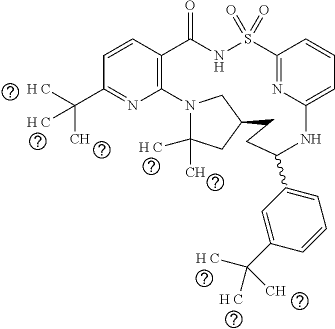 | +++ | ++ | | |
| 41 | 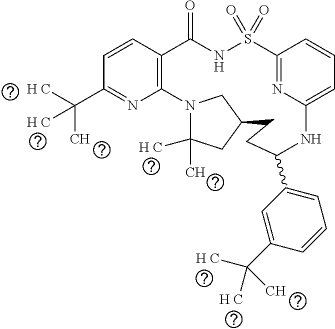 | +++ | +++ | | |
| 42 | 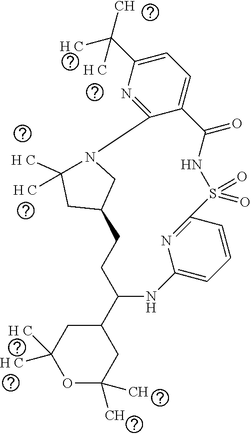 | +++ | ++ | | |
| 43 | 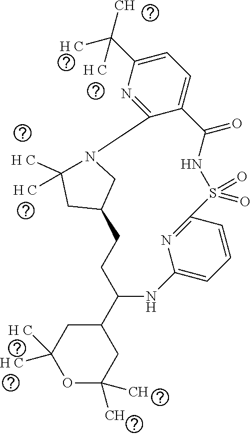 | ND | + | | |
| 44 | 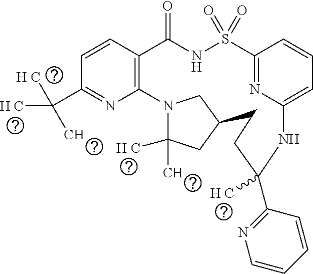 | +++ | ++ | | |
| 45 | 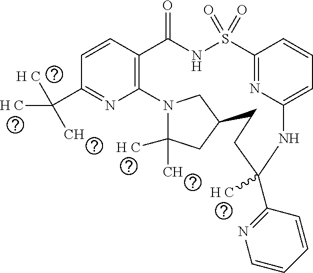 | +++ | ++ | | |
| 46 | 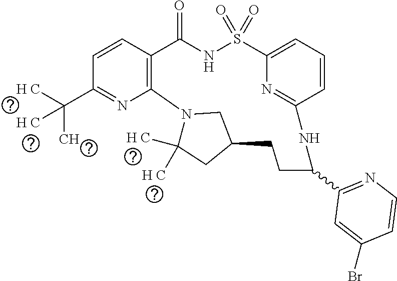 | +++ | +++ | | |
| 47 | 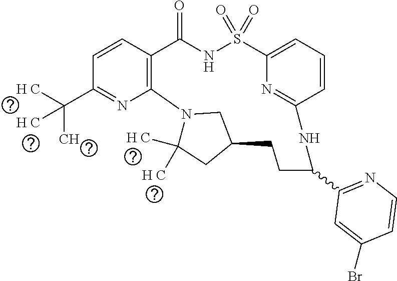 | +++ | ++ | | |
| 48 | 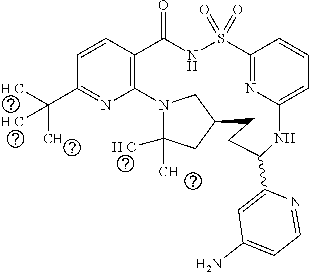 | ++ | ++ | | |
| 49 | 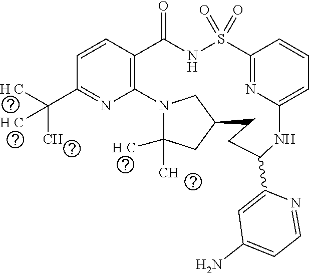 | +++ | ++ | | |
| 50 | 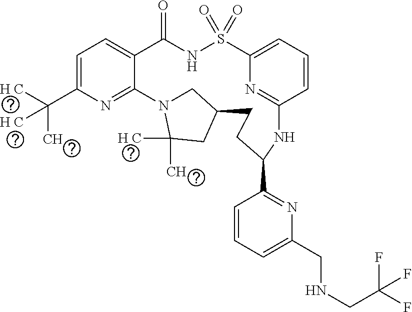 | ++ | +++ | | |
| 51 | 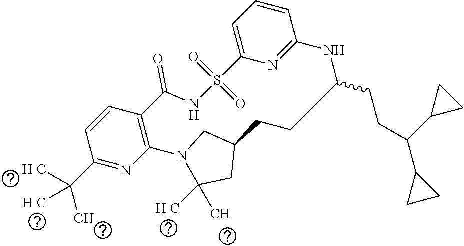 | + | + | | |
| 52 | 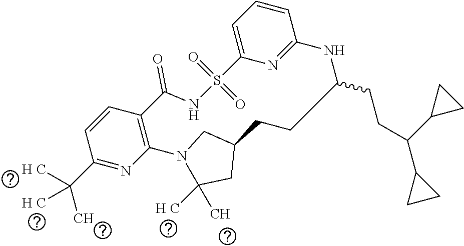 | +++ | ++ | | |
| 53 | 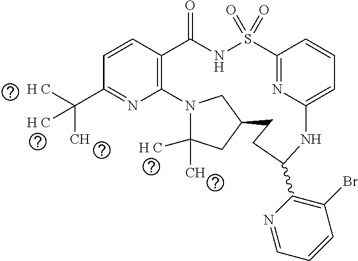 | +++ | +++ | | |
| 54 | 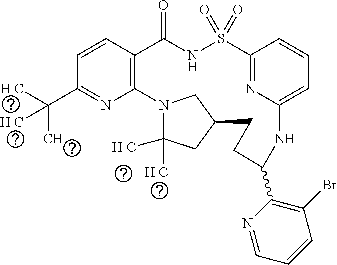 | + | + | | |
| 55 | 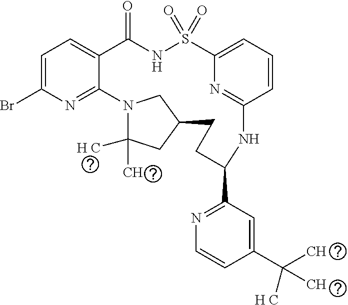 | +++ | ++ | | |
| 56 | 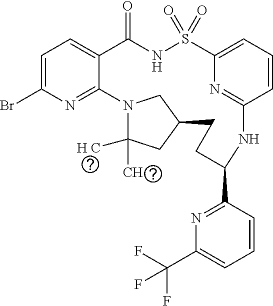 | + | + | | |
| 57 | 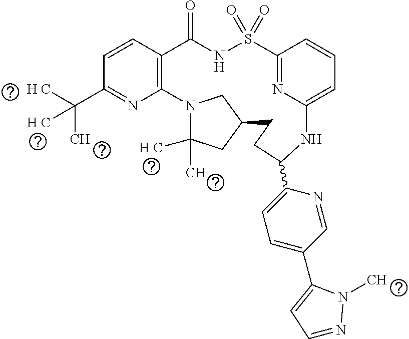 | +++ | ++ | | |
| 58 | 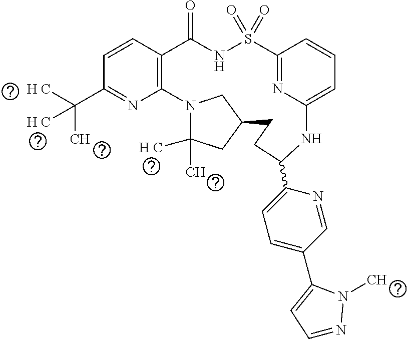 | +++ | +++ | | |
| 59 | 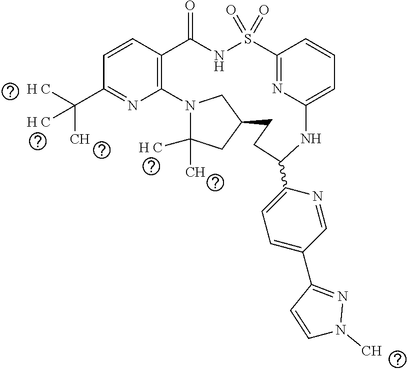 | +++ | + | | |
| 60 | 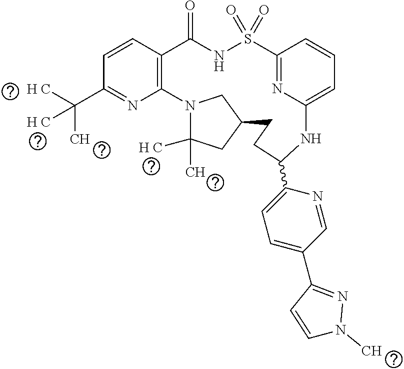 | +++ | +++ | | |
| 61 | 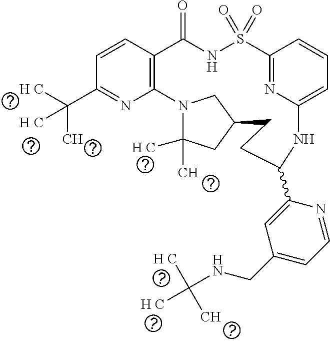 | +++ | +++ | | |
| 62 | 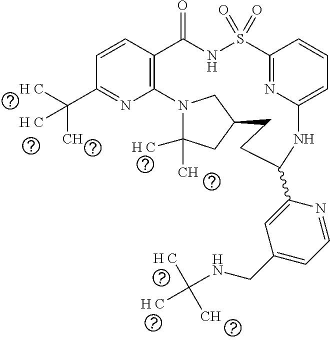 | ++ | ++ | | |
| 63 | 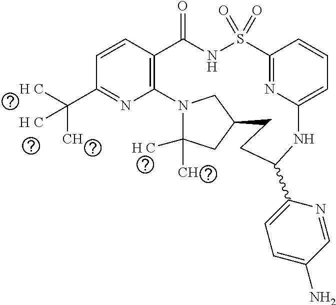 | +++ | +++ | | |
| 64 | 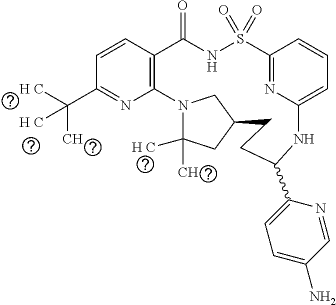 | +++ | ++ | | |
| 65 | 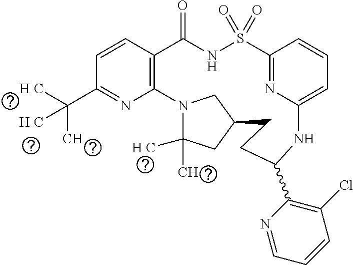 | +++ | ++ | | |
| 66 | 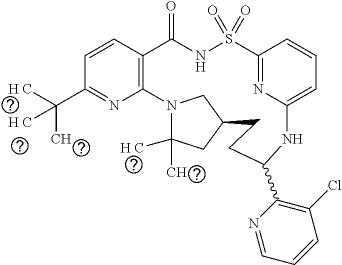 | + | + | | |
| 67 | 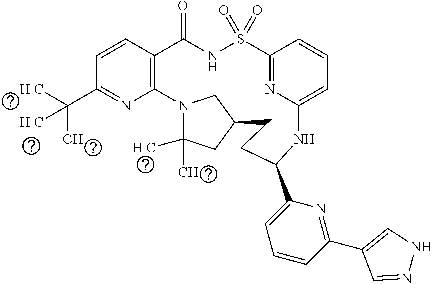 | +++ | +++ | | |
| 68 | 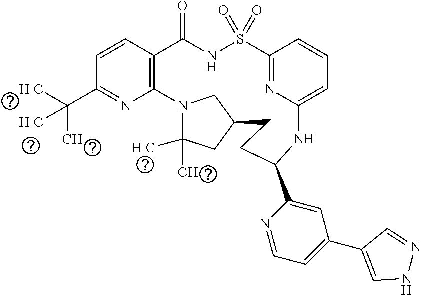 | +++ | +++ | | |
| 69 | 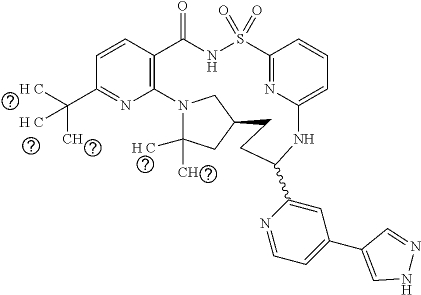 | +++ | ++ | | |
| 70 | 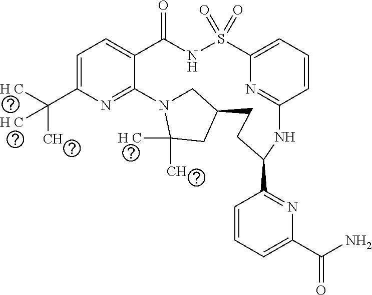 | +++ | ++ | | |
| 71 | 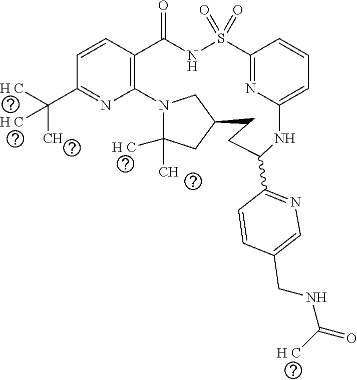 | +++ | ++ | | |
| 72 | 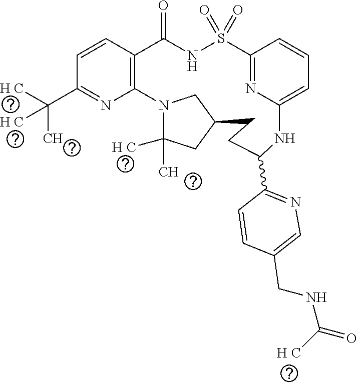 | +++ | ++ | | |
| 73 | 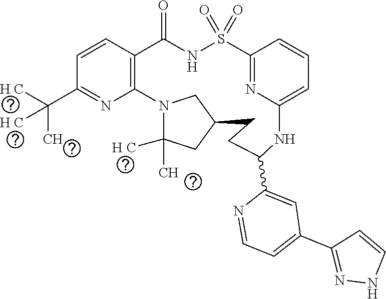 | +++ | +++ | | |
| 74 | 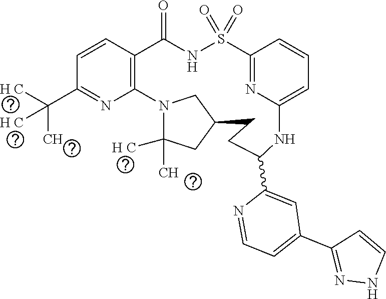 | +++ | ++ | | |
| 75 | 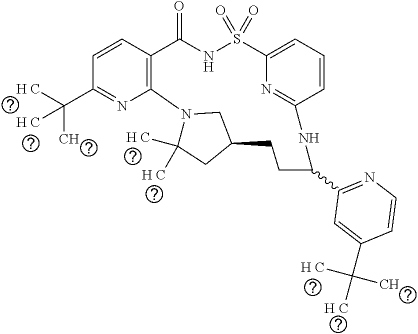 | +++ | +++ | | |
| 76 | 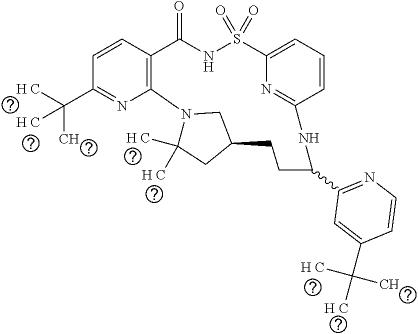 | +++ | ++ | | |
| 77 | 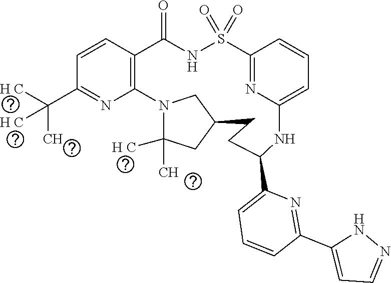 | +++ | ++ | | |
| 78 | 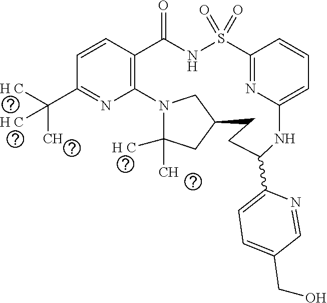 | +++ | +++ | | |
| 79 | 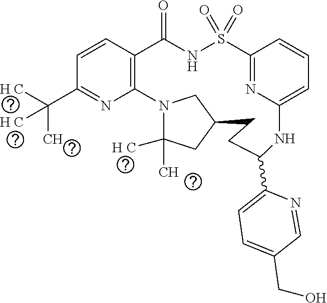 | +++ | +++ | | |
| 80 | 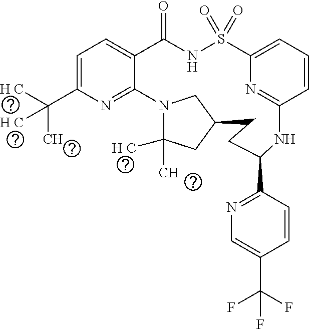 | +++ | ++ | | |
| 81 | 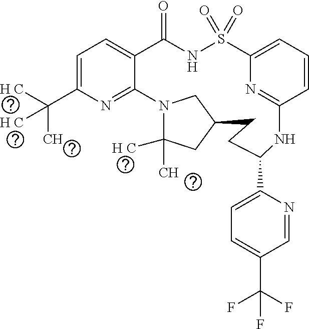 | +++ | + | | |
| 82 | 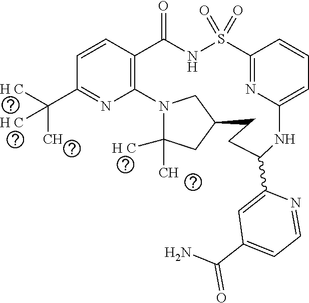 | +++ | ++ | | |
| 83 | 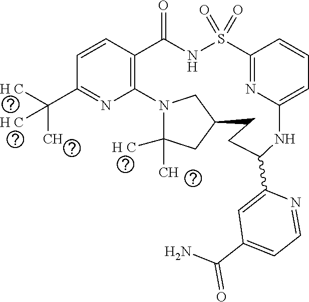 | +++ | ++ | | |
| 84 | 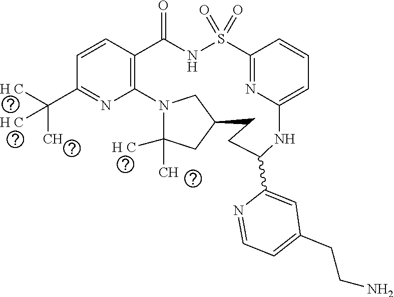 | +++ | +++ | | |
| 85 | 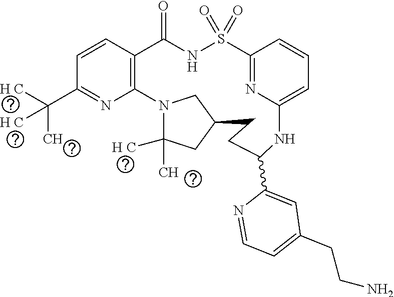 | +++ | ++ | | |
| 86 | 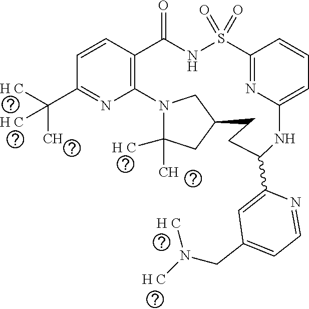 | +++ | ++ | | |
| 87 | 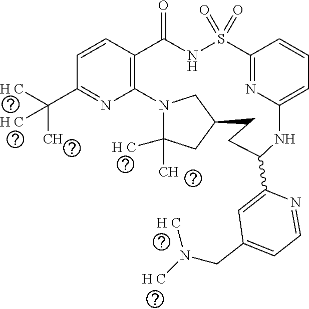 | +++ | + | | |
| 88 | 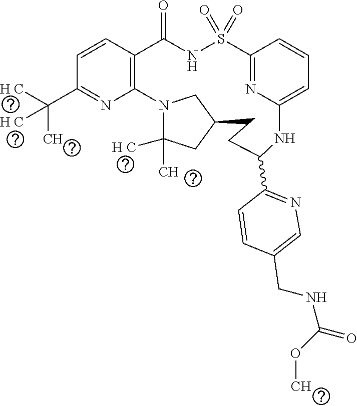 | +++ | +++ | | |
| 89 | 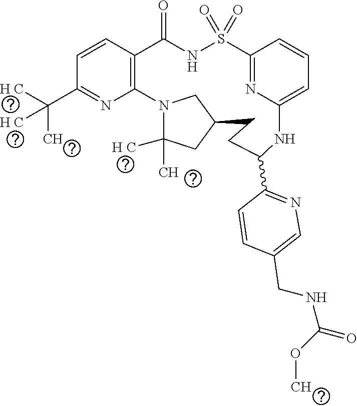 | +++ | ++ | | |
| 90 | 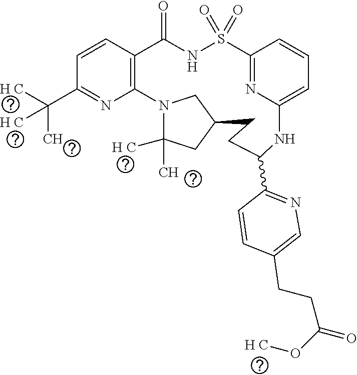 | +++ | ++ | | |
| 91 | 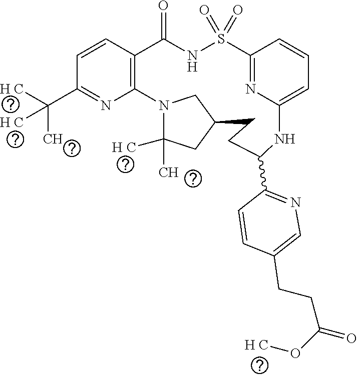 | + | ++ | | |
| 92 | 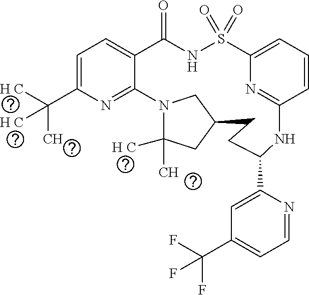 | +++ | 52.5 | | |
| 93 | 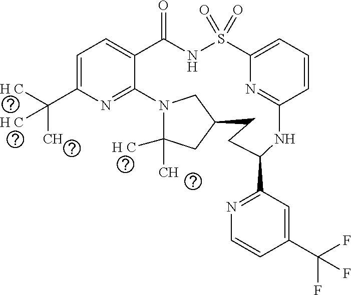 | +++ | +++ | | |
| 94 | 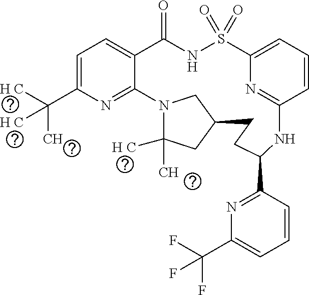 | +++ | ++ | | |
| 95 | 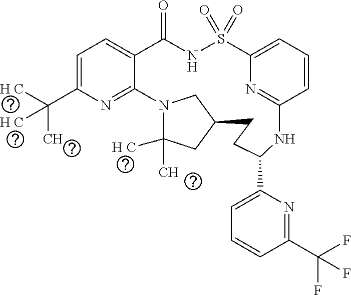 | +++ | ++ | | |
| 96 | 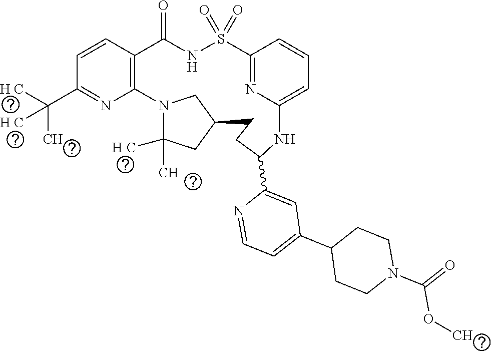 | +++ | +++ | | |
| 97 | 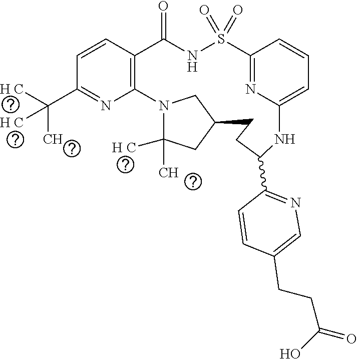 | ++ | ++ | | |
| 98 | 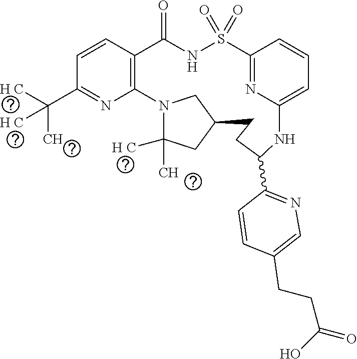 | ++ | + | | |
| 99 | 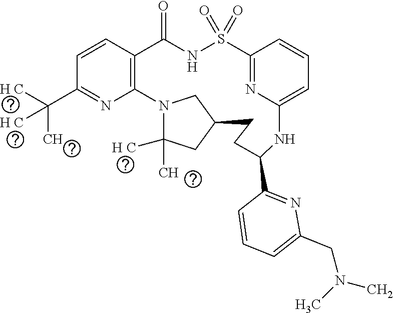 | +++ | ++ | | |
| 100 | 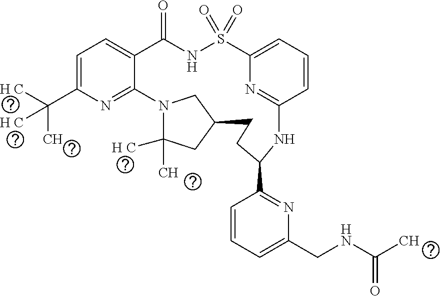 | +++ | +++ | | |
| 101 | 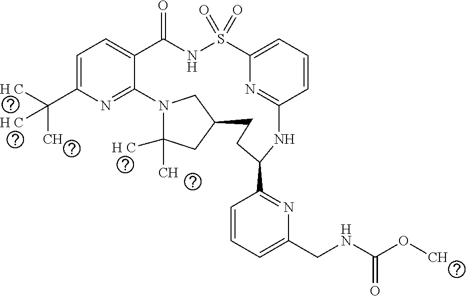 | +++ | ++ | | |
| 102 | 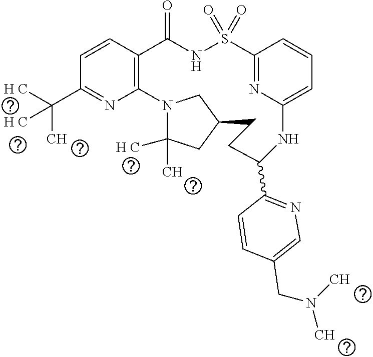 | +++ | +++ | | |
| 103 | 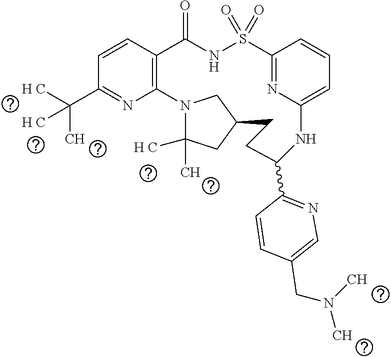 | +++ | ++ | | |
| 104 | 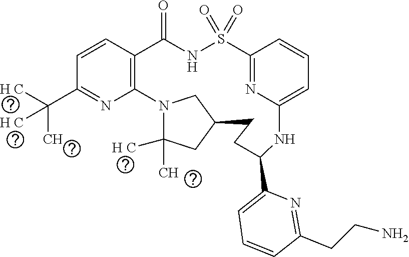 | +++ | ++ | | |
| 105 | 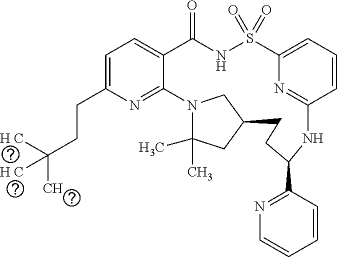 | +++ | +++ | | |
| 106 | 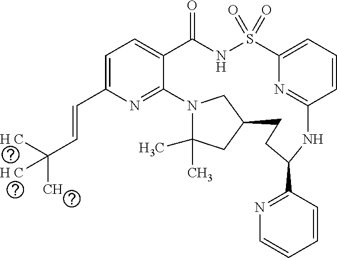 | +++ | +++ | | |
| 107 | 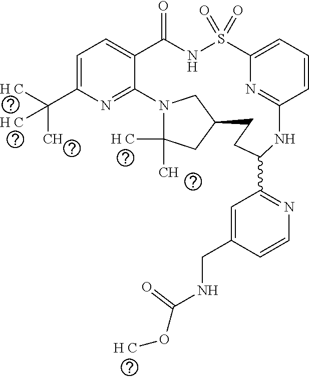 | +++ | ++ | | |
| 108 | 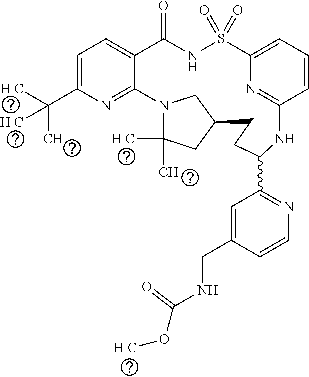 | +++ | ++ | | |
| 109 | 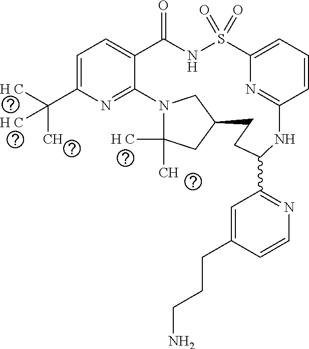 | +++ | +++ | | |
| 110 | 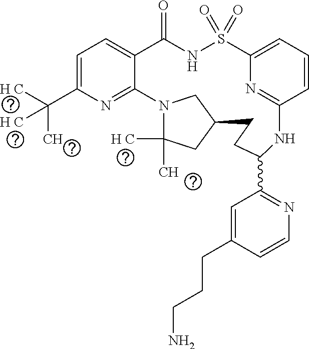 | + | + | | |
| 111 | 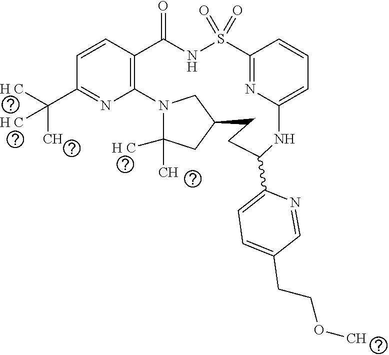 | +++ | ++ | | |
| 112 | 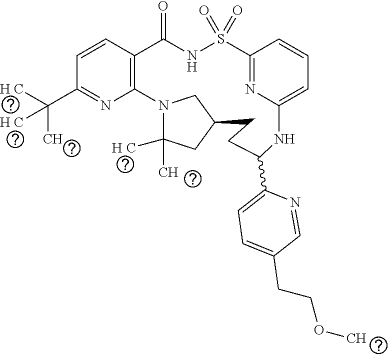 | +++ | ++ | | |
| 113 | 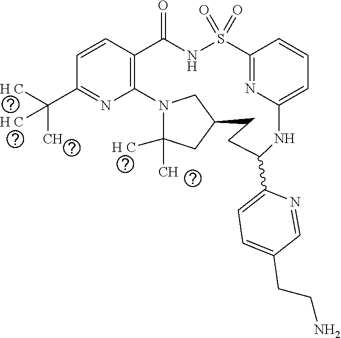 | +++ | ++ | | |
| 114 | 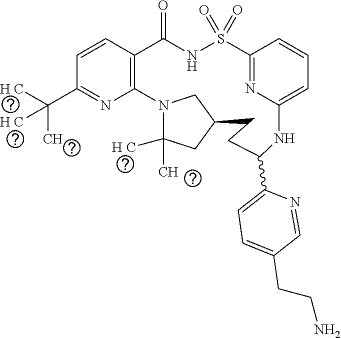 | +++ | ++ | | |
| 115 | 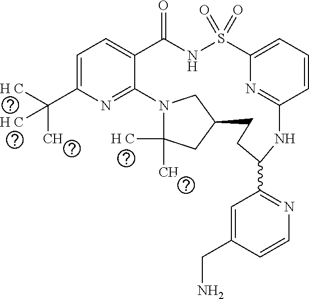 | +++ | +++ | | |
| 116 | 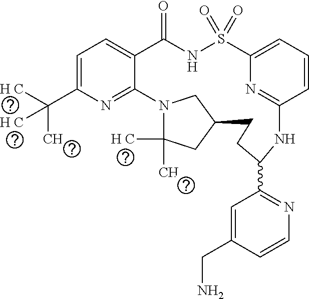 | +++ | +++ | | |
| 117 | 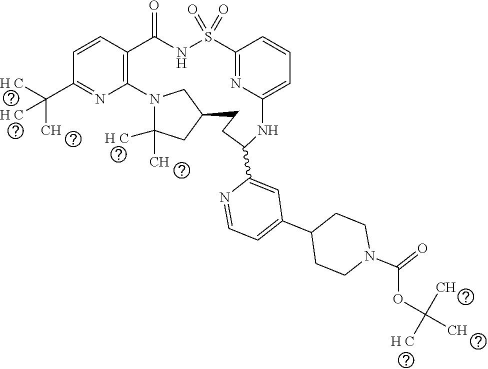 | +++ | +++ | | |
| 118 | 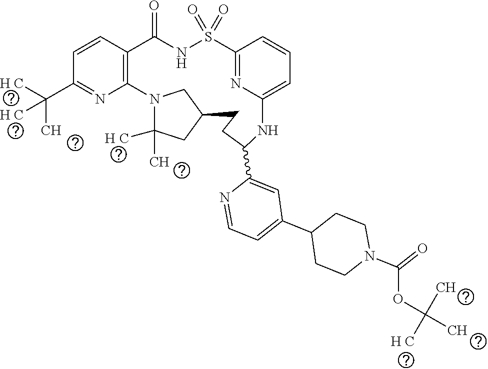 | +++ | ++ | | |
| 119 | 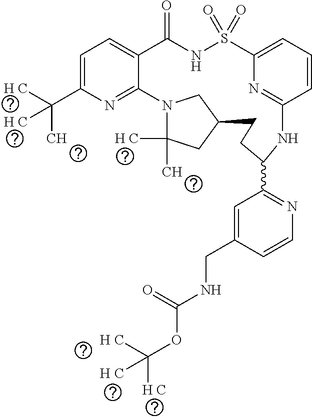 | +++ | +++ | | |
| 120 | 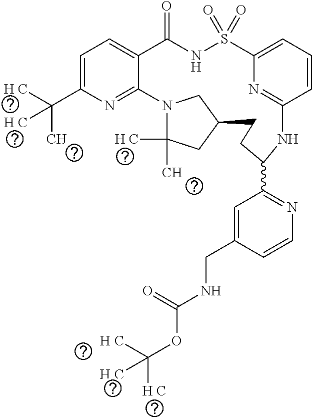 | +++ | +++ | | |
| 121 | 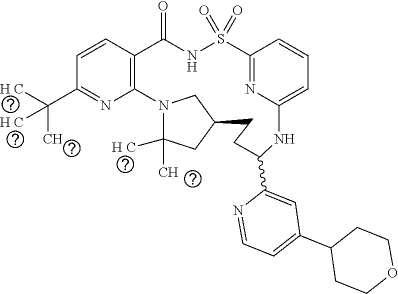 | +++ | +++ | | |
| 122 | 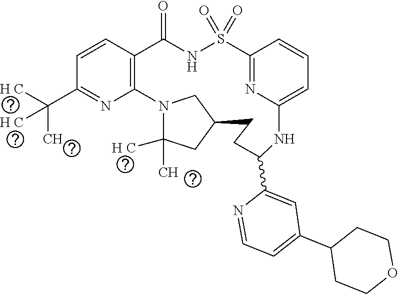 | +++ | ++ | | |
| 123 | 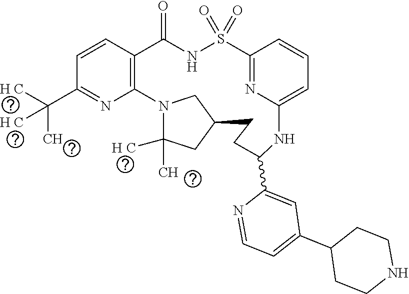 | +++ | +++ | | |
| 124 | 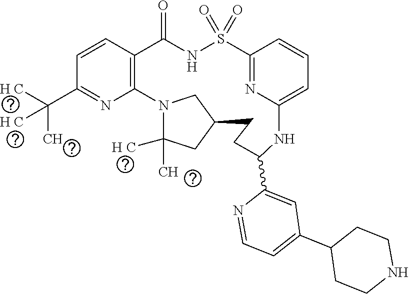 | +++ | ++ | | |
| 125 | 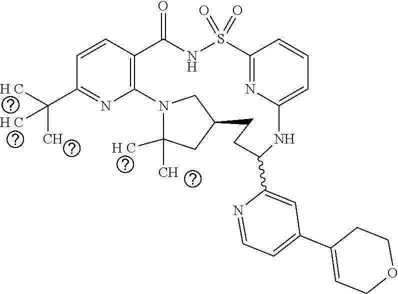 | +++ | +++ | | |
| 126 | 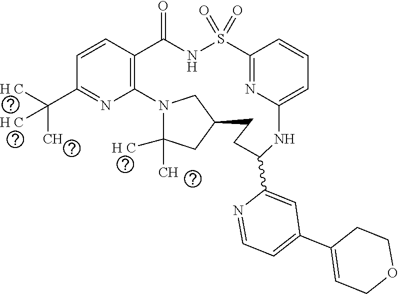 | +++ | +++ | | |
| 127 | 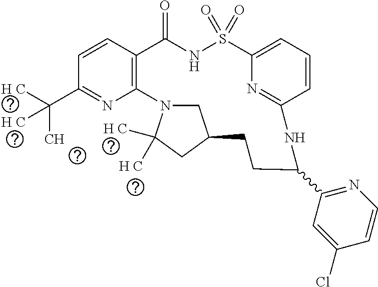 | +++ | +++ | | |
| 128 | 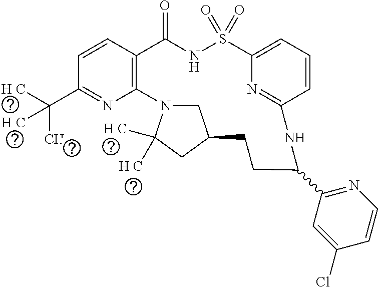 | +++ | +++ | | |
| 129 | 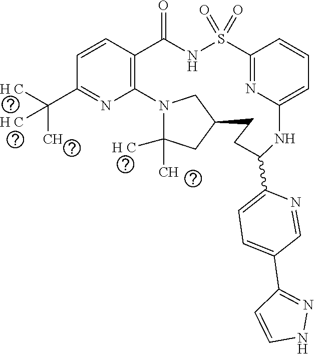 | +++ | +++ | | |
| 130 | 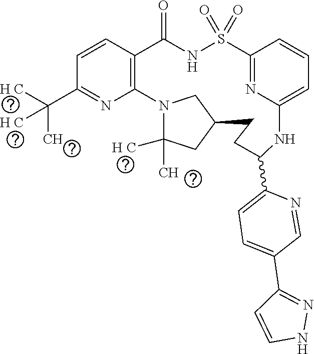 | +++ | +++ | | |
| 131 | 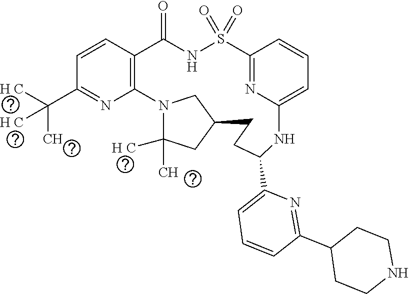 | + | + | | |
| 132 | 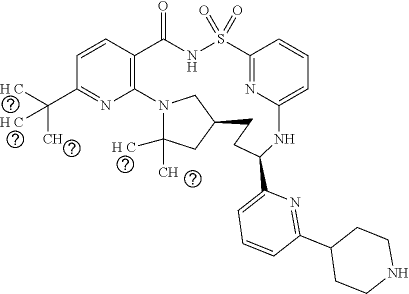 | +++ | ++ | | |
| 133 | 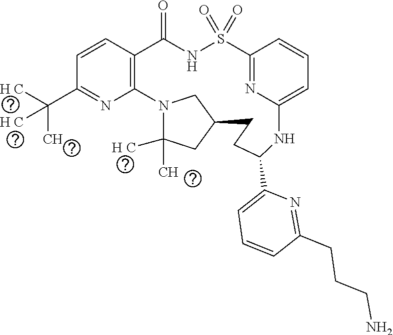 | +++ | + | | |
| 134 | 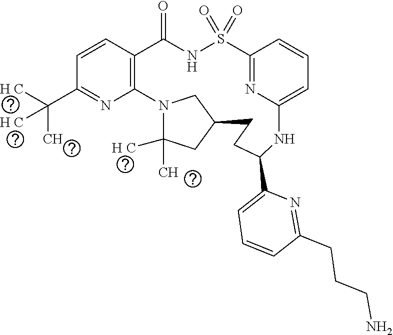 | +++ | +++ | | |
| 135 | 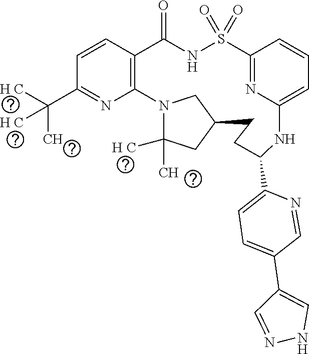 | +++ | ++ | | |
| 136 | 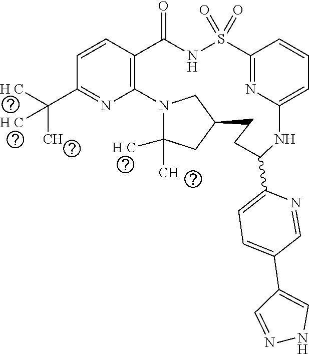 | +++ | +++ | | |
| 137 | 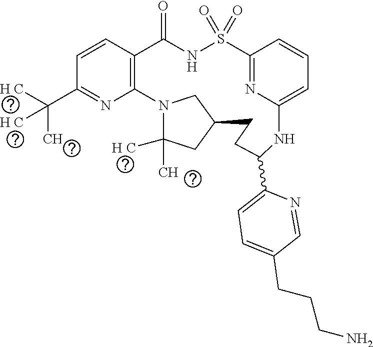 | +++ | ++ | | |
| 138 | 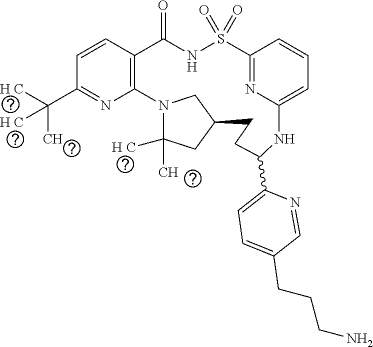 | +++ | ++ | | |
| 139 | 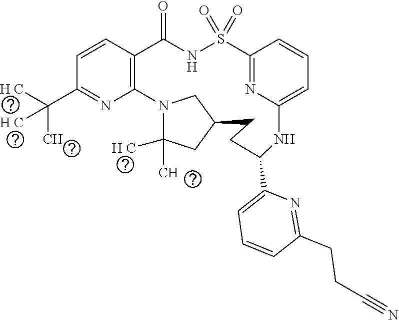 | + | + | | |
| 140 | 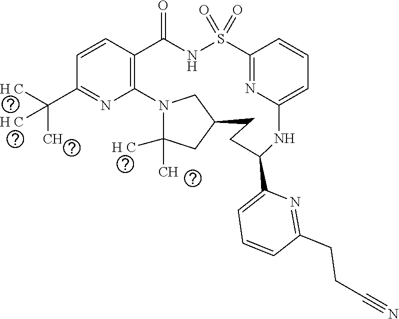 | +++ | ++ | | |
| 141 | 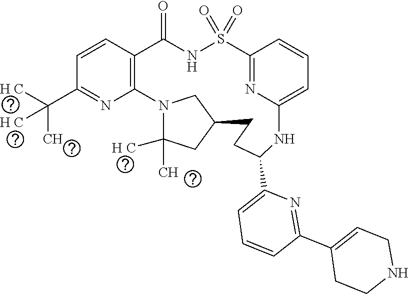 | ++ | ++ | | |
| 142 | 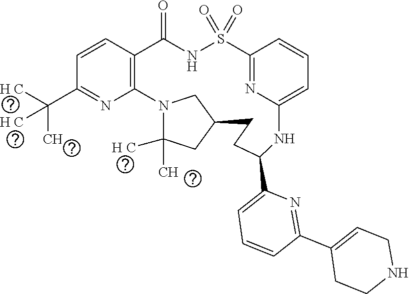 | +++ | +++ | | |
| 143 | 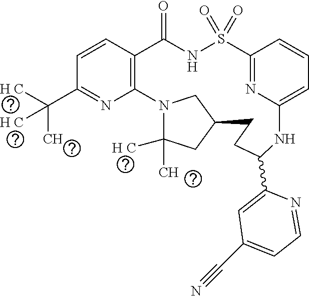 | +++ | ++ | | |
| 144 | 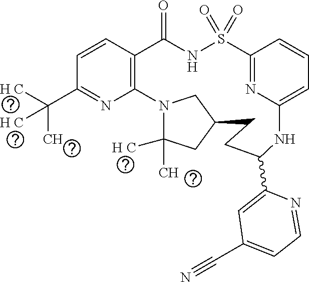 | +++ | +++ | | |
| 145 | 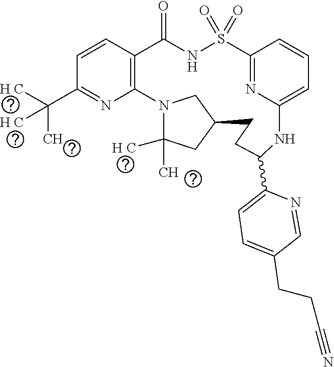 | +++ | ++ | | |
| 146 | 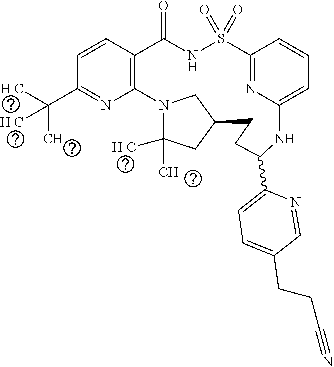 | +++ | +++ | | |
| 147 | 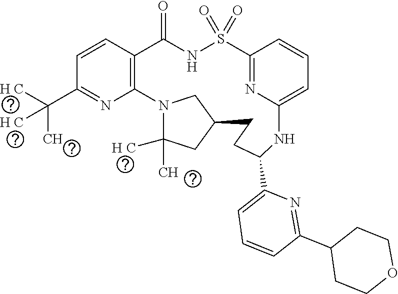 | + | + | | |
| 148 | 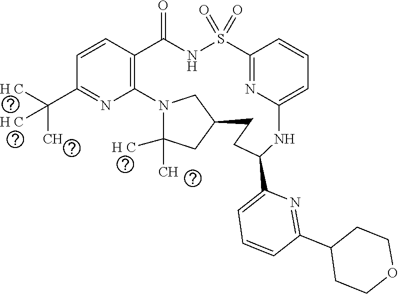 | +++ | +++ | | |
| 149 | 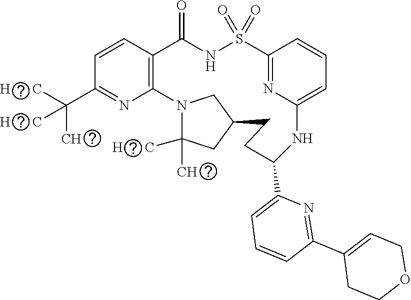 | +++ | + | | |
| 150 | 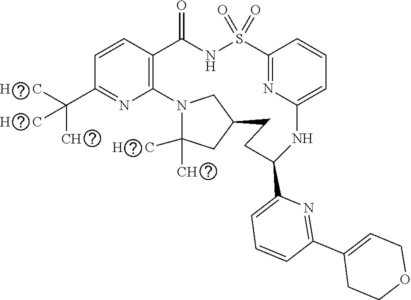 | +++ | +++ | | |
| 151 | 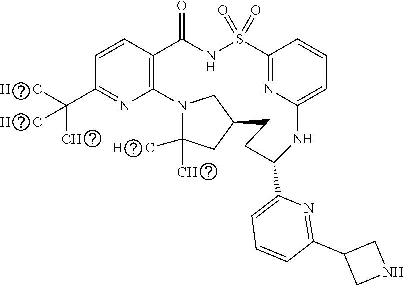 | +++ | ++ | | |
| 152 | 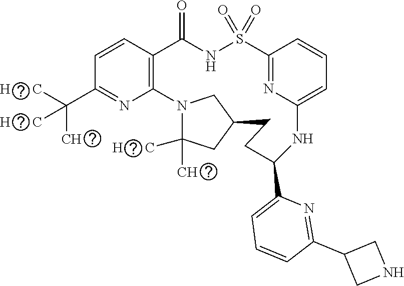 | +++ | +++ | | |
| 153 | 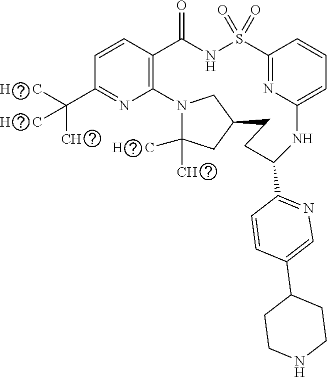 | +++ | ++ | | |
| 154 | 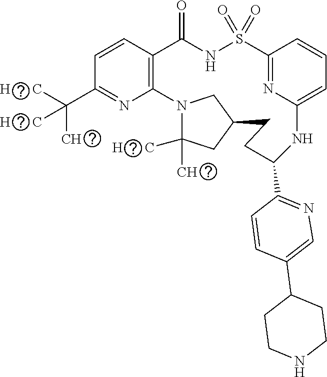 | +++ | +++ | | |
| 155 | 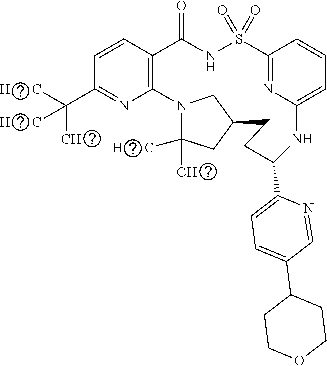 | +++ | +++ | | |
| 156 | 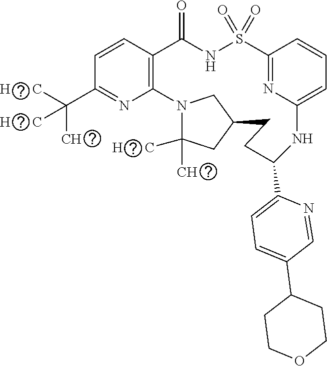 | +++ | ++ | | |
| 157 | 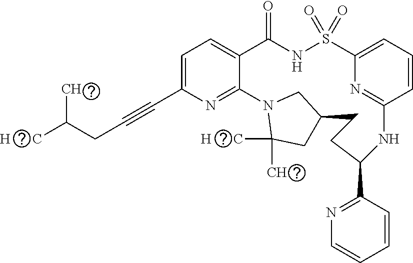 | +++ | +++ | | |
| 158 | 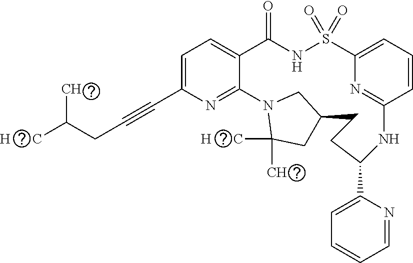 | +++ | ++ | | |
| 159 | 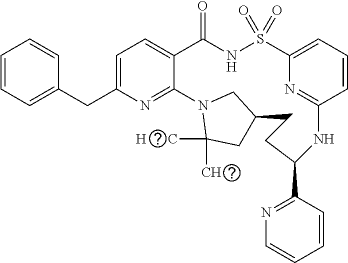 | +++ | +++ | | |
| 160 | 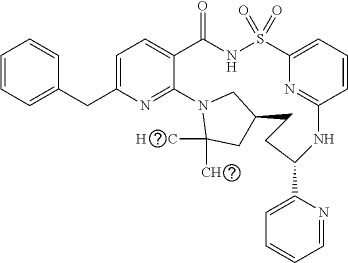 | +++ | ++ | | |
| 161 | 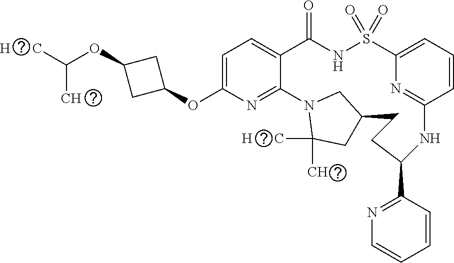 | +++ | +++ | | |
| 162 | 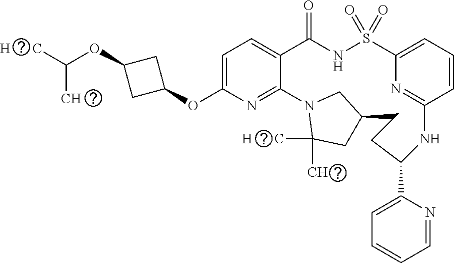 | +++ | ++ | | |
| 163 | 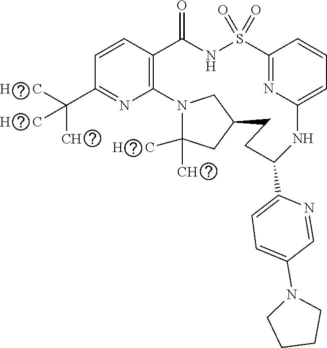 | +++ | ++ | | |
| 164 | 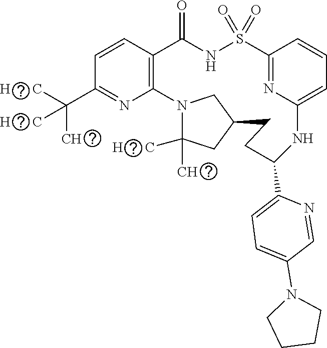 | +++ | +++ | | |
| 165 | 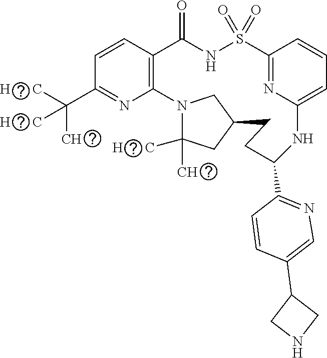 | +++ | ++ | | |
| 166 | 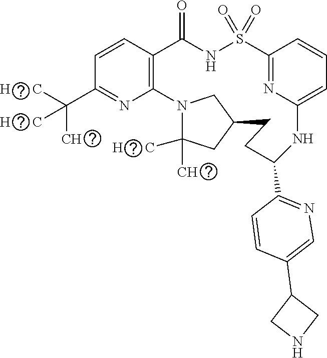 | +++ | +++ | | |
| 167 | 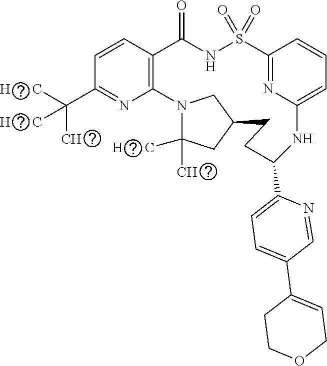 | +++ | ++ | | |
| 168 | 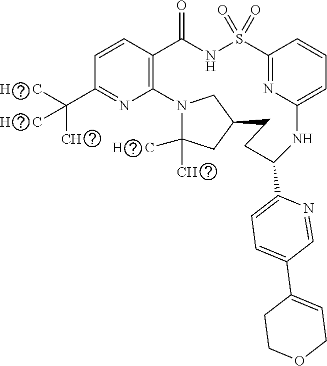 | +++ | +++ | | |
| 169 | 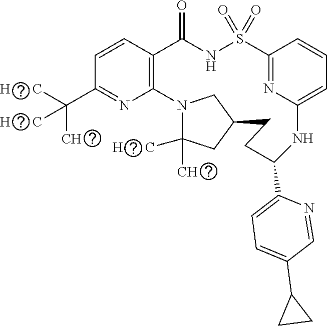 | +++ | ++ | | |
| 170 | 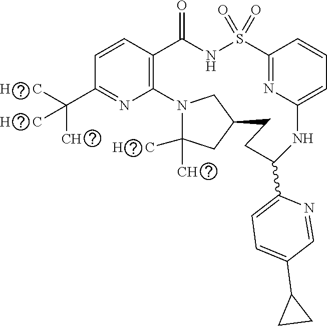 | +++ | +++ | | |
| 171 | 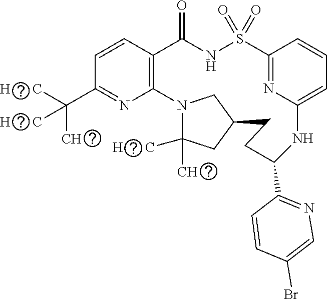 | +++ | ++ | | |
| 172 | 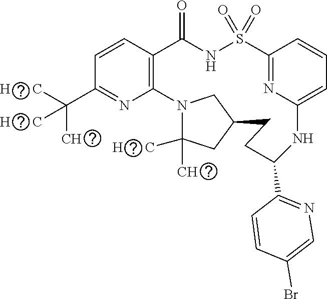 | +++ | +++ | | |
| 173 | 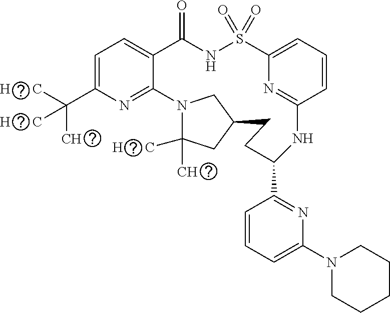 | +++ | + | | |
| 174 | 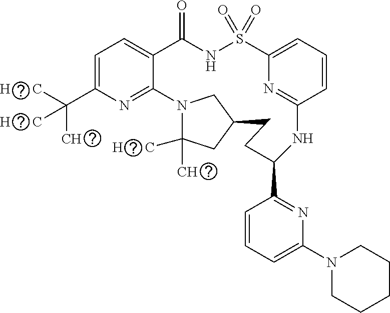 | +++ | +++ | | |
| 175 | 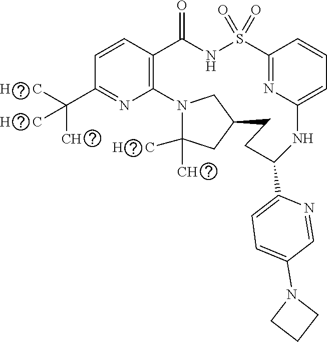 | +++ | +++ | | |
| 176 | 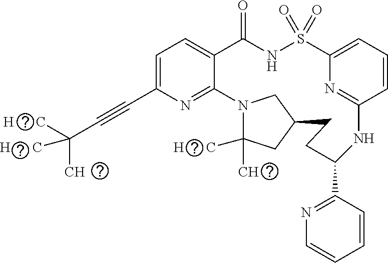 | +++ | ++ | | |
| 177 | 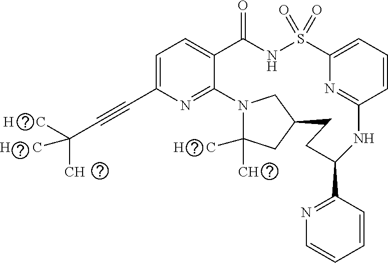 | +++ | +++ | | |
| 178 | 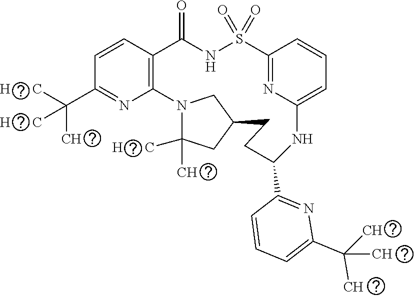 | +++ | + | | |
| 179 | 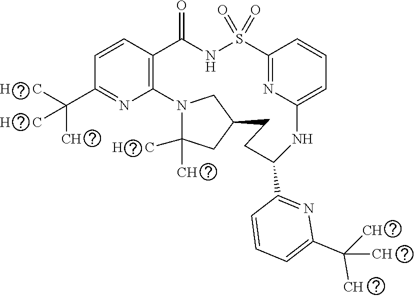 | +++ | ++ | | |
| 180 | 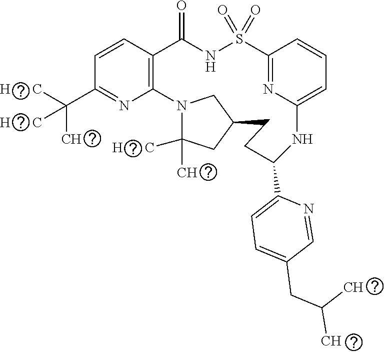 | +++ | +++ | | |
| 181 | 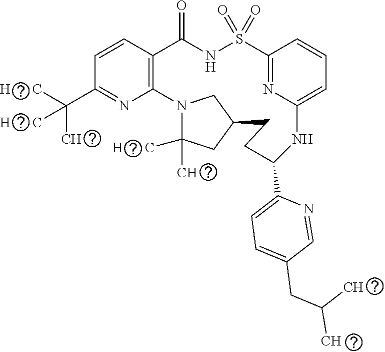 | +++ | ++ | | |
| 182 | 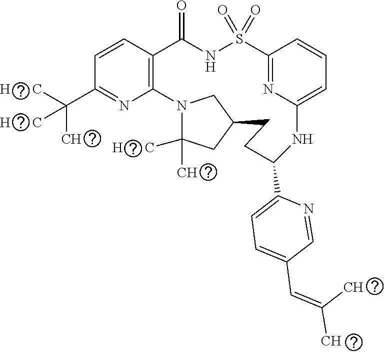 | +++ | +++ | | |
| 183 | 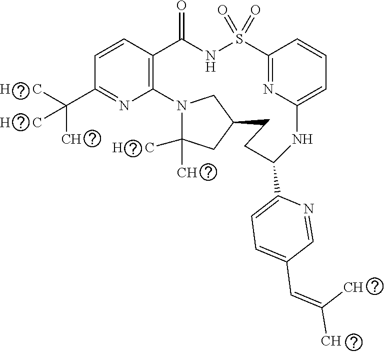 | +++ | ++ | | |
| 184 | 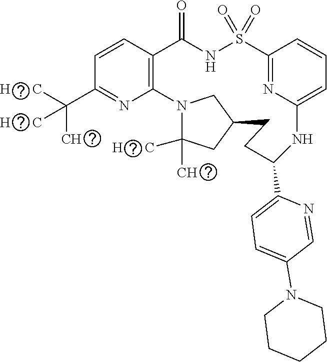 | +++ | +++ | | |
| 185 | 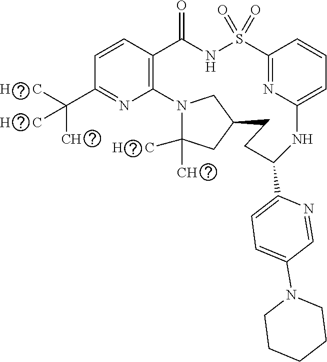 | +++ | ++ | | |
| 186 | 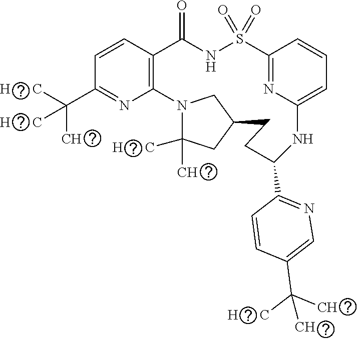 | +++ | +++ | | |
| 187 | 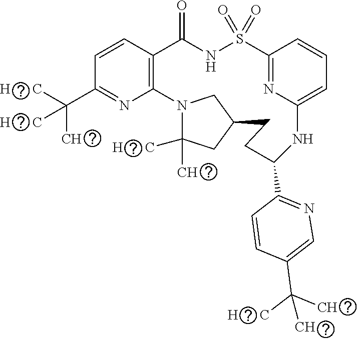 | +++ | ++ | | |
| 188 | 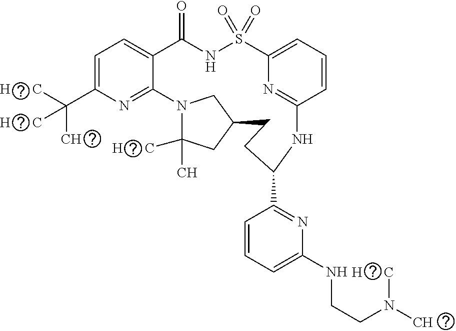 | +++ | ++ | | |
| 189 | 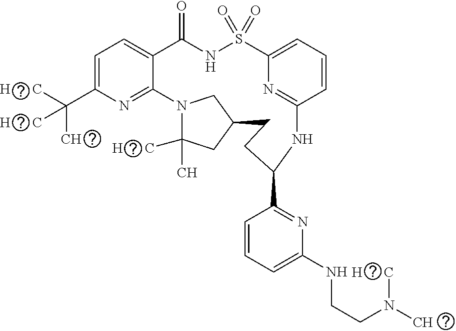 | +++ | ++ | | |
| 190 | 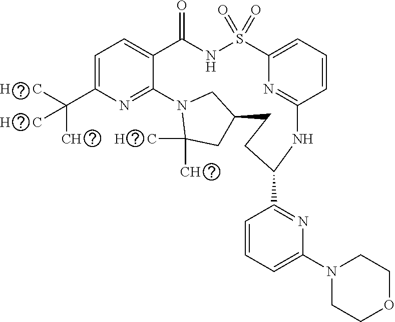 | +++ | ++ | | |
| 191 | 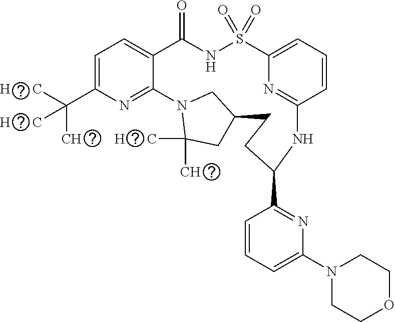 | +++ | +++ | | |
| 192 | 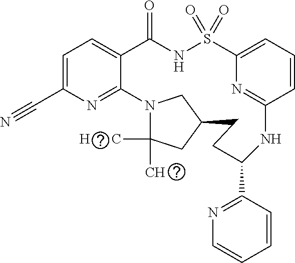 | ++ | + | | |
| 193 | 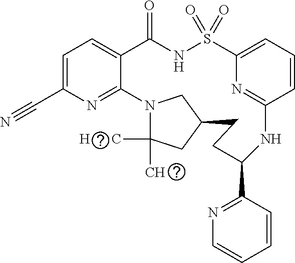 | + | +++ | | |
| 194 | 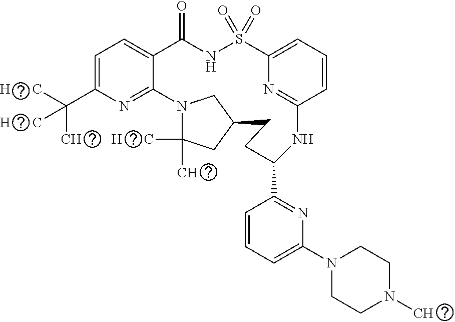 | +++ | ++ | | |
| 195 | 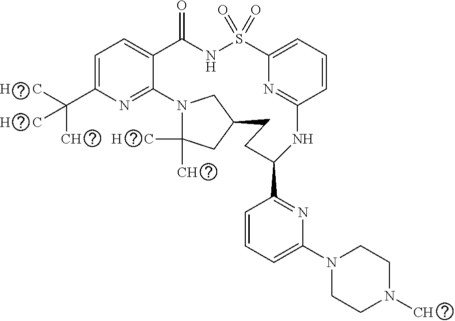 | +++ | +++ | | |
| 196 | 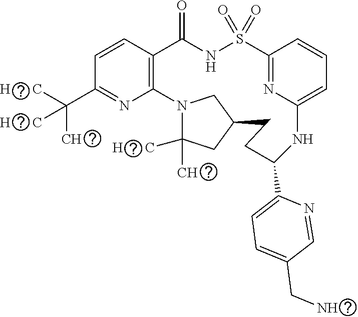 | +++ | +++ | | |
| 197 | 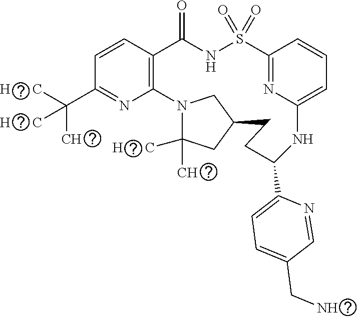 | +++ | ++ | | |
| 198 | 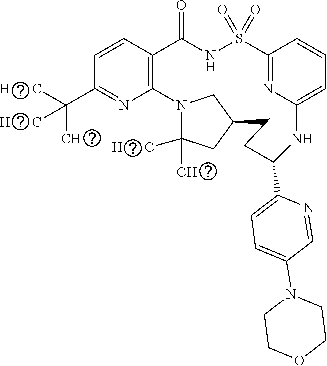 | +++ | +++ | | |
| 199 | 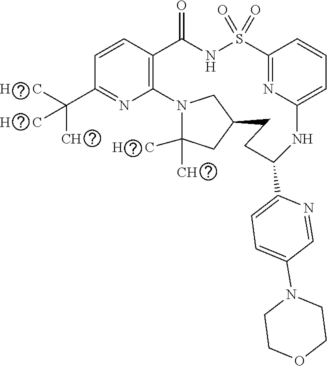 | +++ | +++ | | |
| 200 | 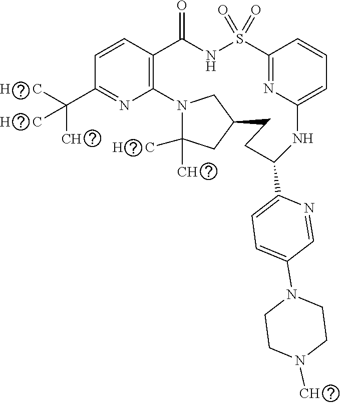 | +++ | +++ | | |
| 201 | 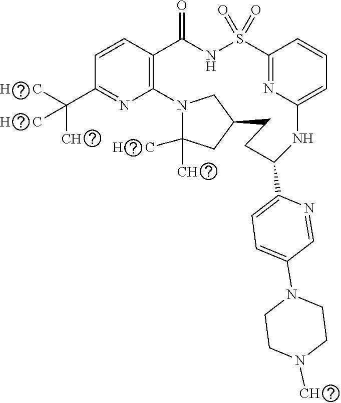 | +++ | +++ | | |
| 202 | 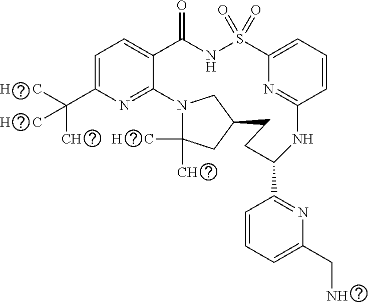 | +++ | ++ | | |
| 203 | 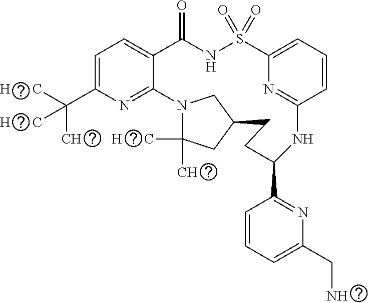 | +++ | +++ | | |
| 204 | 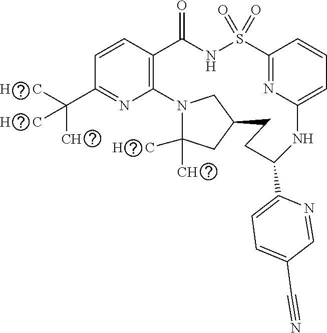 | +++ | +++ | | |
| 205 | 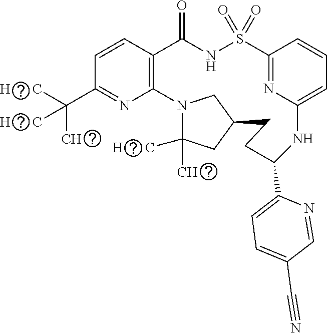 | +++ | +++ | | |
| 206 | 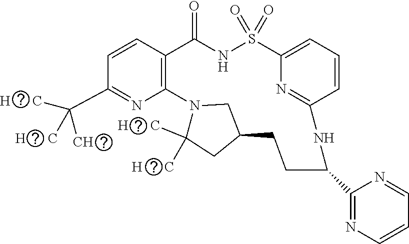 | ++ | +++ | | |
| 207 | 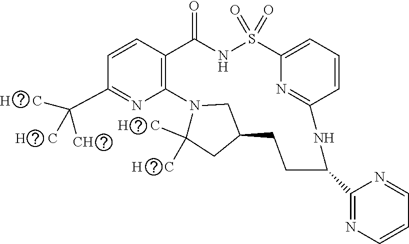 | +++ | +++ | | |
| 208 | 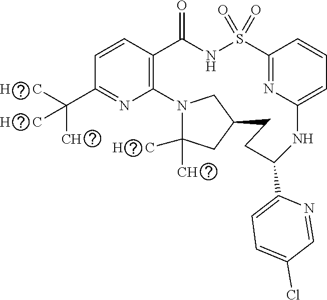 | +++ | +++ | | |
| 209 | 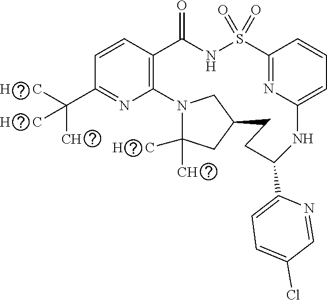 | +++ | ++ | | |
| 210 | 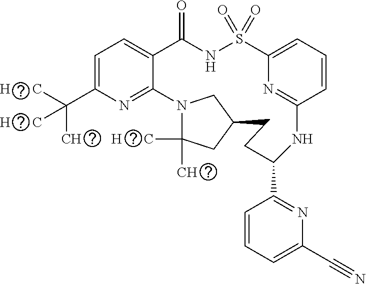 | +++ | ++ | | |
| 211 | 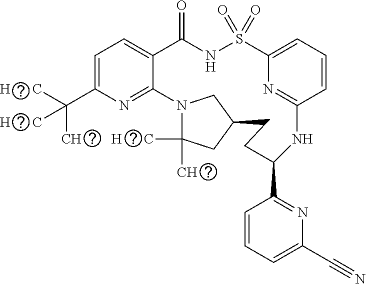 | +++ | +++ | | |
| 212 | 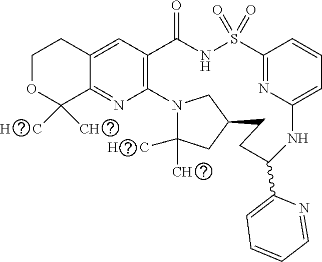 | +++ | +++ | | |
| 213 | 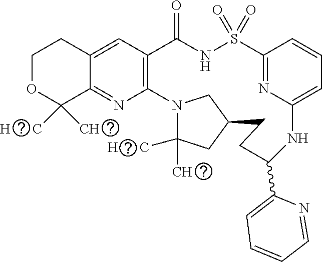 | ++ | ++ | | |
| 214 | 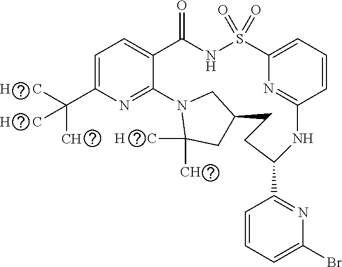 | +++ | ++ | | |
| 215 | 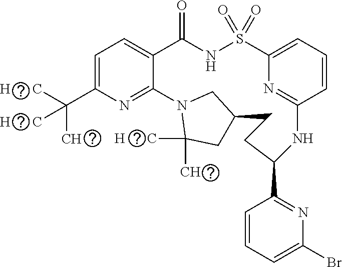 | +++ | +++ | | |
| 216 | 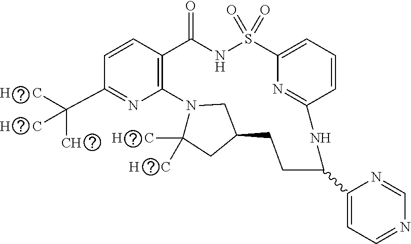 | ND | + | | |
| 217 | 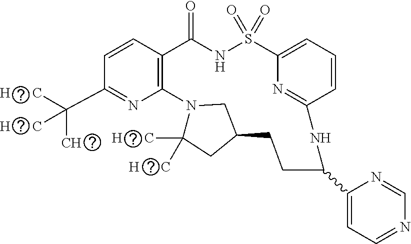 | +++ | ++ | | |
| 218 | 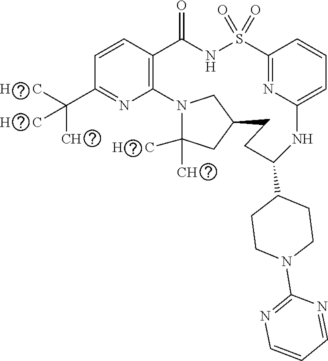 | +++ | ++ | | |
| 219 | 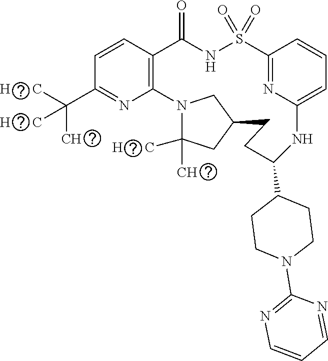 | +++ | ++ | | |
| 220 | 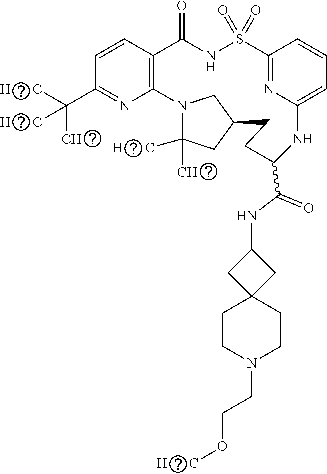 | ++ | +++ | | |
| 221 | 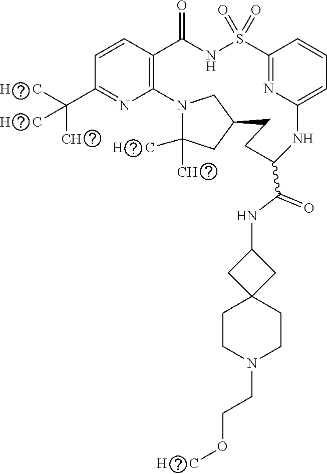 | + | + | | |
| 222 | 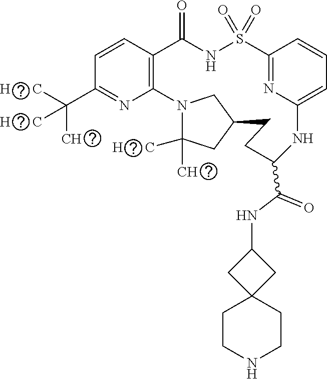 | + | ++ | | |
| 223 | 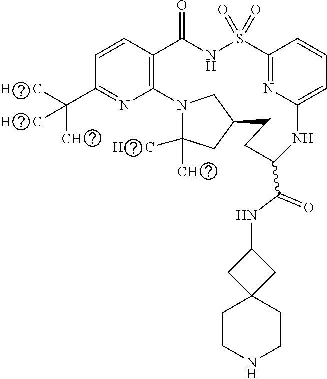 | ND | + | | |
| 224 | 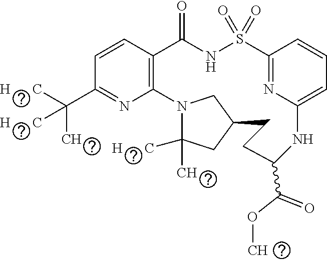 | +++ | +++ | | |
| 225 | 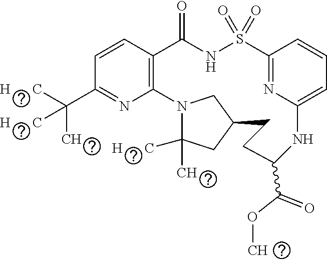 | +++ | ++ | | |
| 226 | 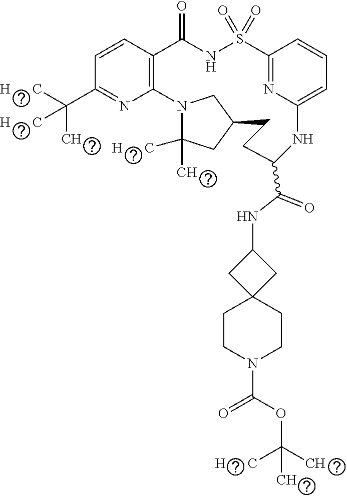 | +++ | +++ | | |
| 227 | 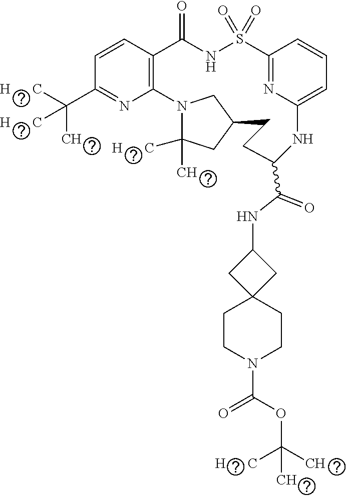 | ++ | + | | |
| 228 | 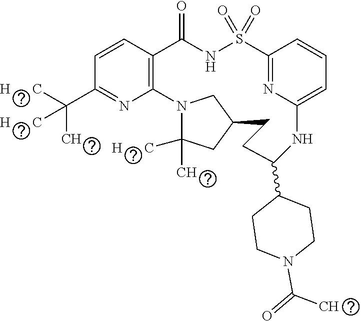 | +++ | ++ | | |
| 229 | 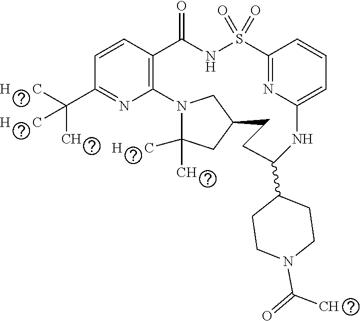 | +++ | ++ | | |
| 230 | 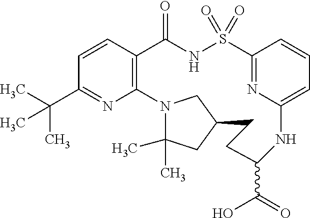 [SMILES: CC(C)(C)c1ccc2c(n1)N1C[C@@H](CCC(C(=O)O)Nc3cccc(n3)S(=O)(=O)NC2=O)CC1(C)C] | + | +++ | | |
| 231 | 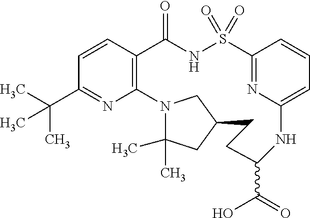 [SMILES: CC(C)(C)c1ccc2c(n1)N1C[C@@H](CCC(C(=O)O)Nc3cccc(n3)S(=O)(=O)NC2=O)CC1(C)C] | + | + | | |
| 232 | 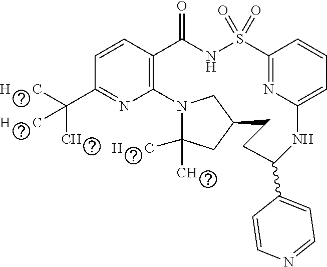 | +++ | +++ | | |
| 233 | 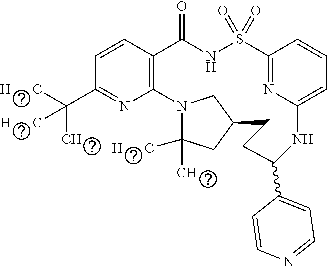 | +++ | ++ | | |
| 234 | 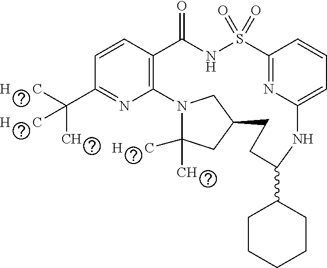 | +++ | ++ | | |
| 235 | 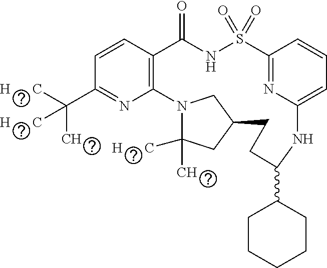 | +++ | ++ | | |
| 236 | 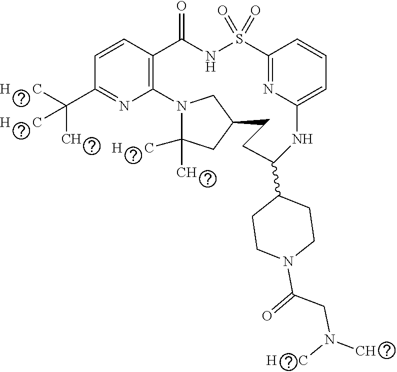 | ++ | ++ | | |
| 237 | 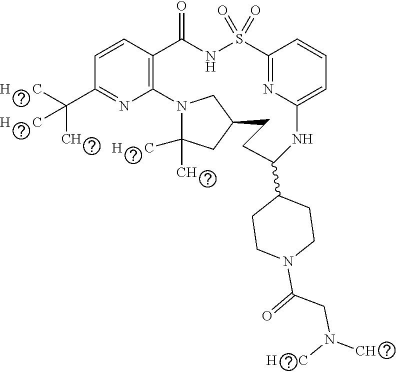 | ++ | ++ | | |
| 238 | 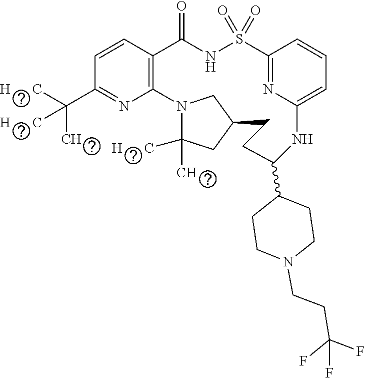 | +++ | ++ | | |
| 239 | 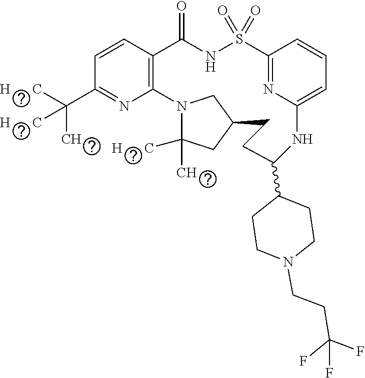 | + | + | | |
| 240 | 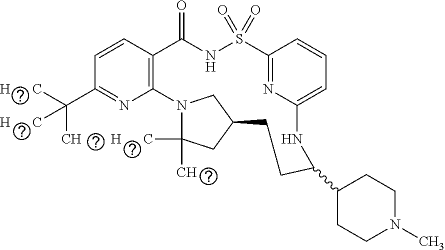 | ++ | +++ | | |
| 241 | 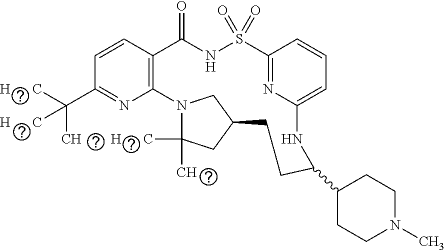 | +++ | ++ | | |
| 242 | 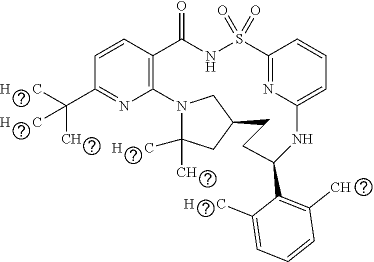 | ND | + | | |
| 243 | 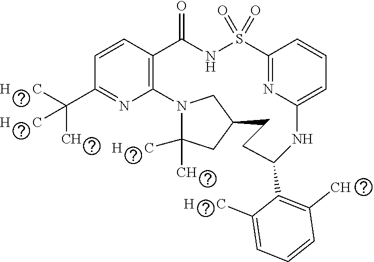 | +++ | +++ | | |
| 244 | 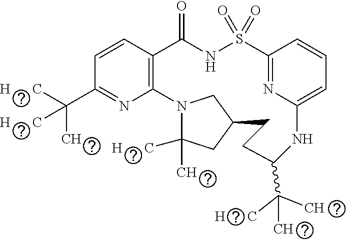 | +++ | +++ | | |
| 245 | 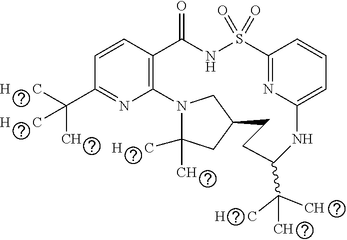 | +++ | ++ | | |
| 246 | 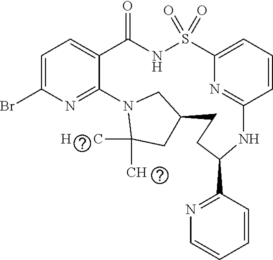 | +++ | ++ | | |
| 247 | 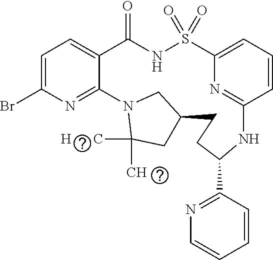 | +++ | + | | |
| 248 | 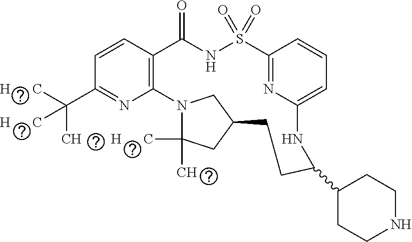 | + | ++ | | |
| 249 | 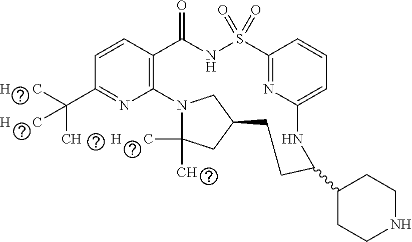 | + | ++ | | |
| 250 | 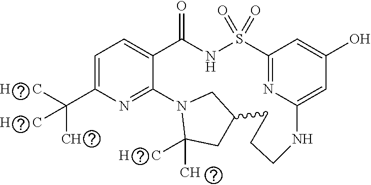 | +++ | ++ | | |
| 251 | 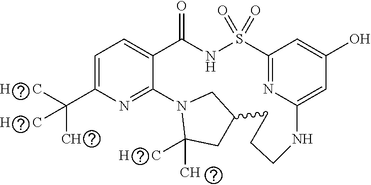 | ND | + | | |
| 252 | 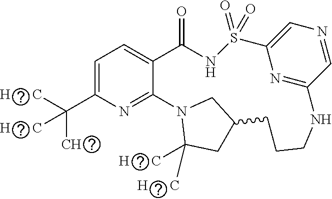 | +++ | ++ | | |
| 253 | 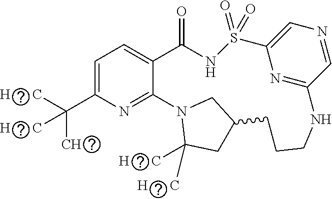 | + | + | | |
| 254 | 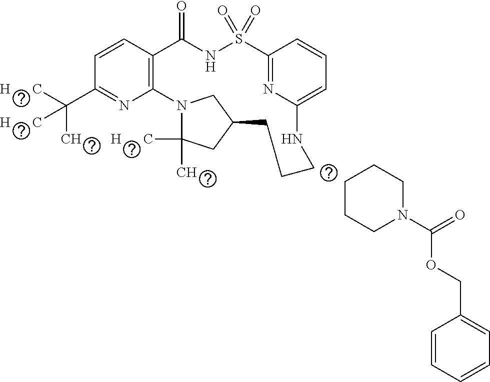 | +++ | + | | |
| 255 | 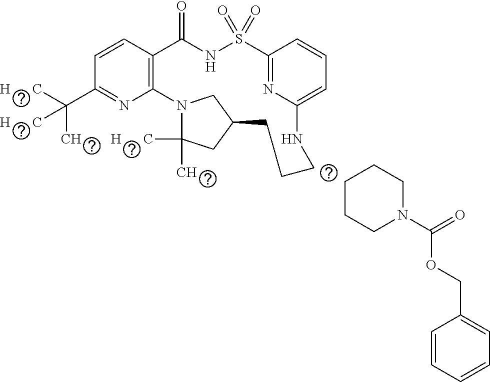 | +++ | +++ | | |
| 256 | 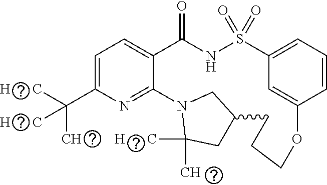 | +++ | ++ | | |
| 257 | 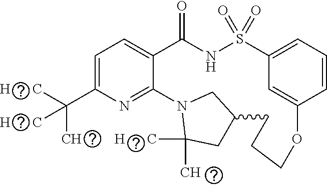 | ND | + | | |
| 258 | 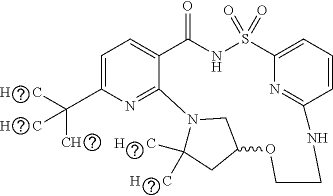 | ND | + | | |
| 259 | 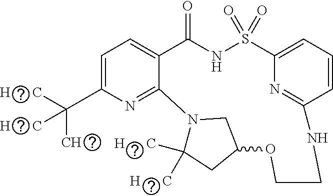 | +++ | ++ | | |
| 260 | 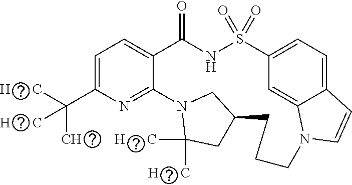 | +++ | + | | |
| 261 | 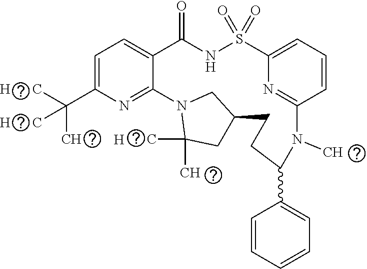 | +++ | + | | |
| 262 | 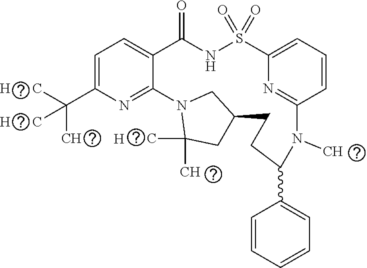 | +++ | +++ | | |
| 263 | 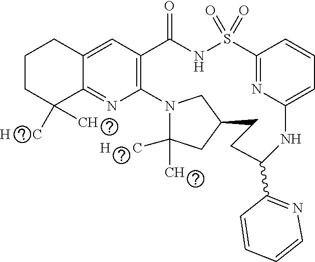 | +++ | +++ | | |
| 264 | 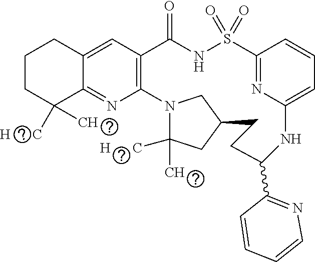 | +++ | ++ | | |
| 265 | 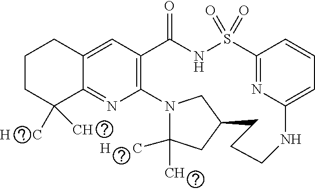 | +++ | ++ | | |
| 266 | 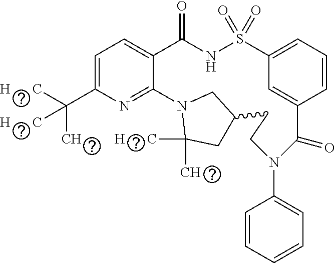 | ND | + | | |
| 267 | 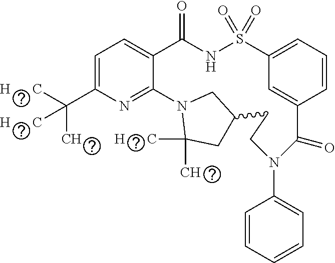 | + | + | | |
| 268 | 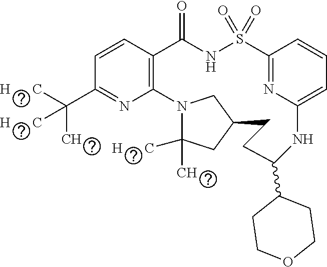 | +++ | +++ | | |
| 269 | 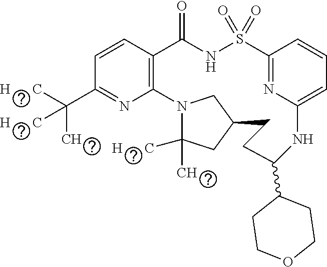 | +++ | ++ | | |
| 270 | 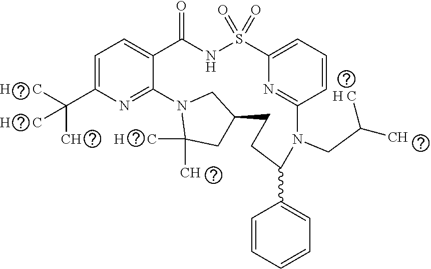 | ND | + | | |
| 271 | 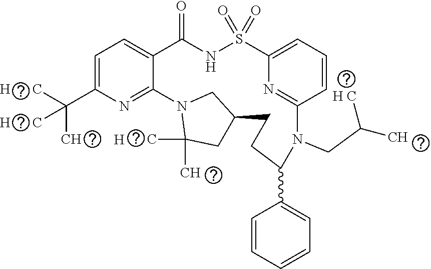 | ND | + | | |
| 272 | 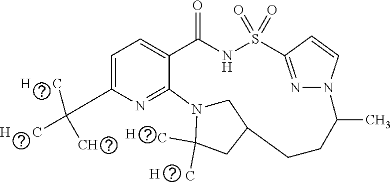 | +++ | +++ | | |
| 273 | 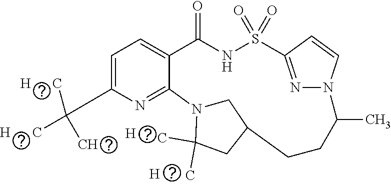 | ++ | ++ | | |
| 274 | 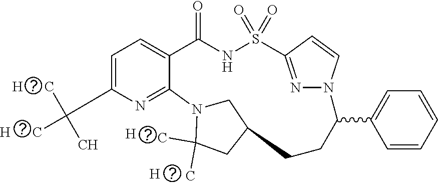 | +++ | ++ | | |
| 275 | 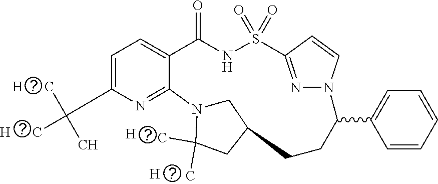 | +++ | ++ | | |
| 276 | 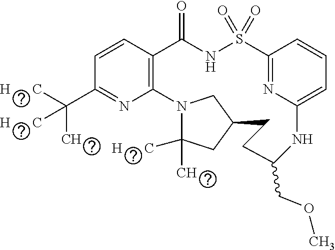 | +++ | ++ | | |
| 277 | 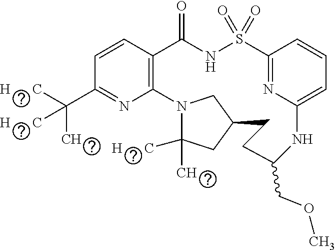 | +++ | +++ | | |
| 278 | 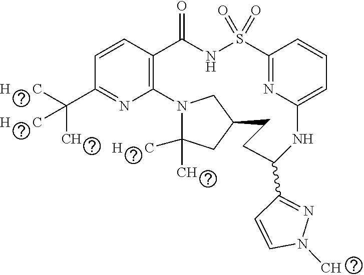 | +++ | ++ | | |
| 279 | 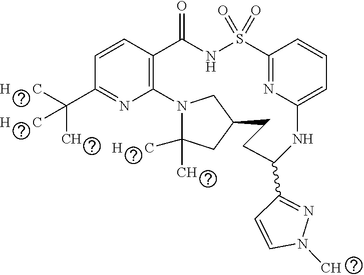 | +++ | +++ | | |
| 280 | 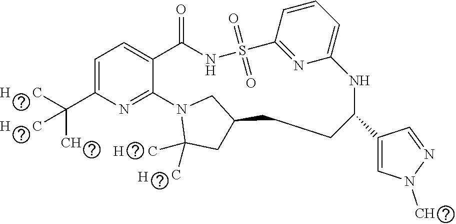 | +++ | +++ | | |
| 281 | 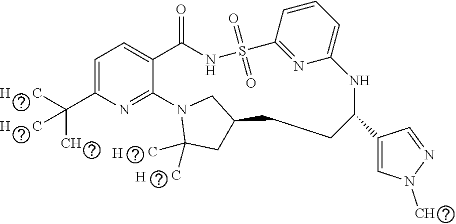 | +++ | +++ | | |
| 282 | 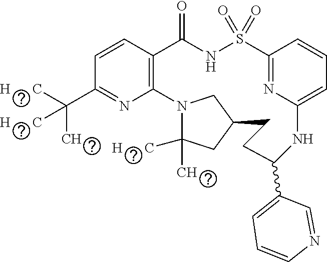 | +++ | +++ | | |
| 283 | 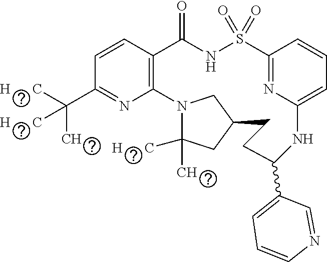 | +++ | +++ | | |
| 284 | 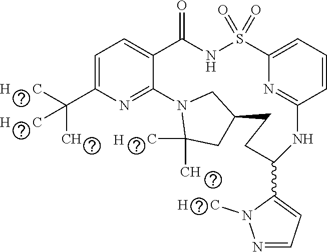 | +++ | ++ | | |
| 285 | 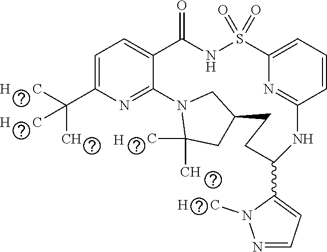 | +++ | +++ | | |
| 286 | 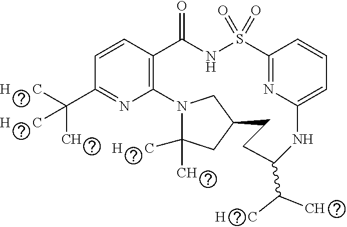 | +++ | +++ | | |
| 287 | 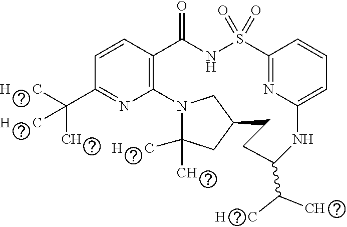 | +++ | +++ | | |
| 288 | 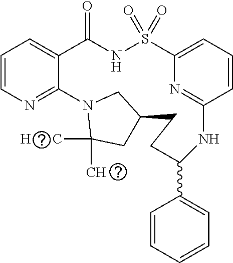 | | | ++ | +++ |
| 289 | 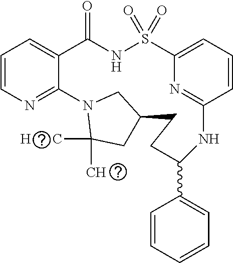 | | | +++ | +++ |
| 290 | 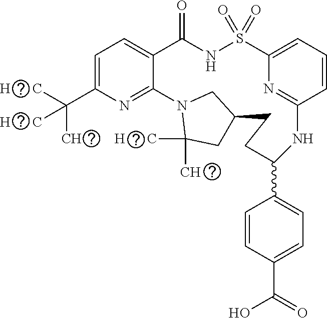 | | | ++ | ++ |
| 291 | 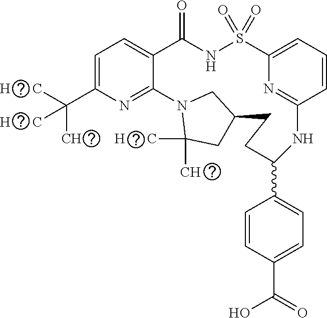 | | | ++ | ++ |
| 292 | 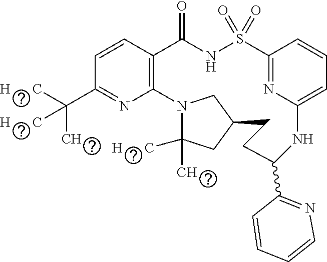 | +++ | +++ | +++ | +++ |
| 293 | 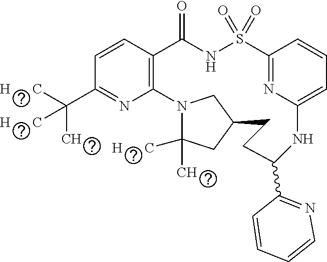 | +++ | ++ | +++ | +++ |
| 294 | 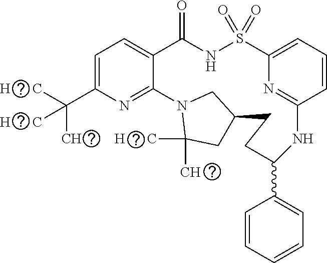 | +++ | +++ | +++ | +++ |
| 295 | 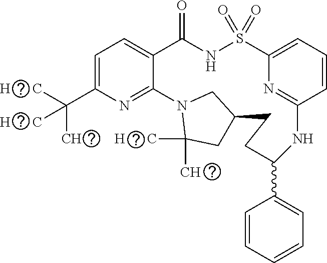 | +++ | ++ | +++ | +++ |
| 296 | 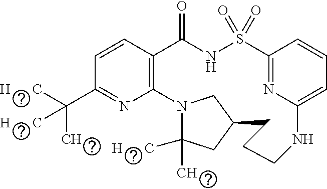 | +++ | +++ | +++ | +++ |
| 297 | 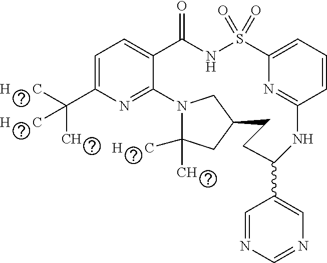 | +++ | +++ | +++ | +++ |
| 298 | 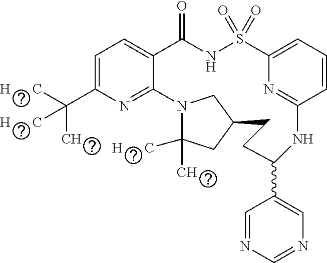 | | | +++ | +++ |

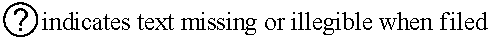

TABLE 3B
Bioactivity for Compounds 299-397

| | | Corrector | Corrector | Corrector | Corrector |
|---|---|---|---|---|---|
| | | assay A | assay A | assay B | assay B |
| Compound | | EC₅₀ | % | EC₅₀ | % |
| Number | Structure | (μM) | activity | (μM) | activity |
| 299 | 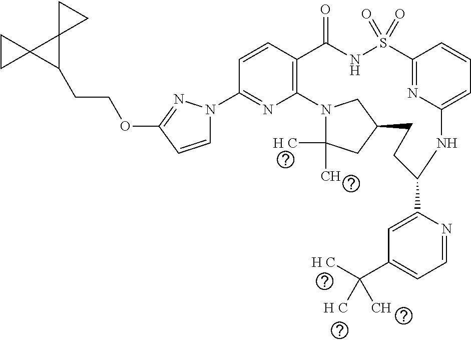 | | | | |
| 300 | 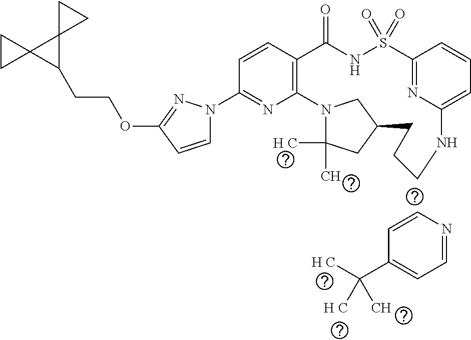 | | | | |
| 301 | 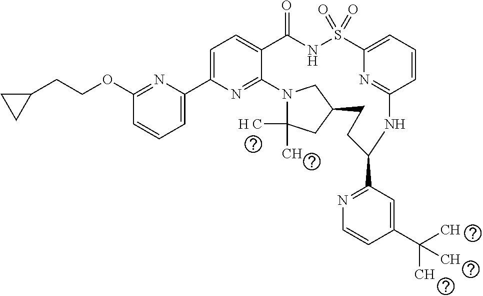 | +++ | +++ | | |
| 302 | 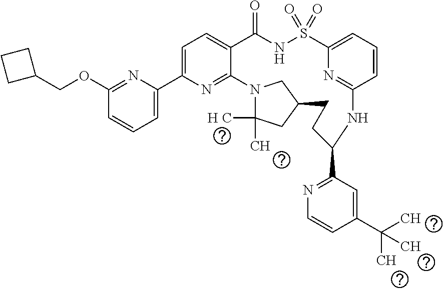 | +++ | +++ | | |
| 303 | 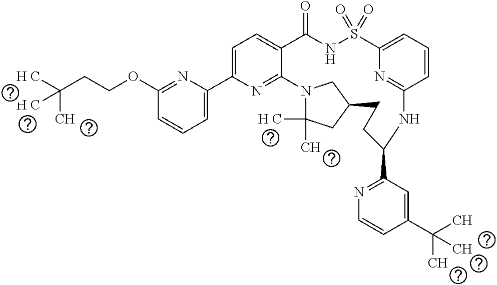 | +++ | +++ | | |
| 304 | 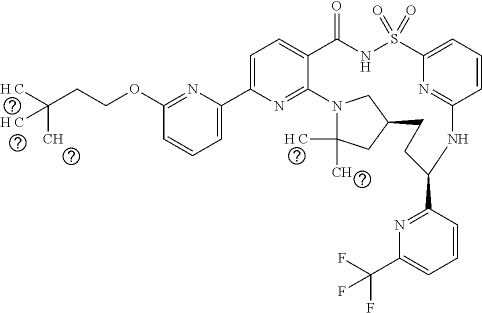 | +++ | ++ | | |
| 305 | 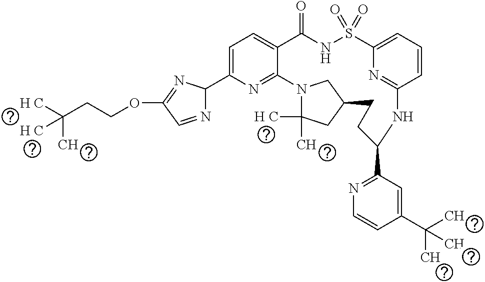 | +++ | +++ | | |
| 306 | 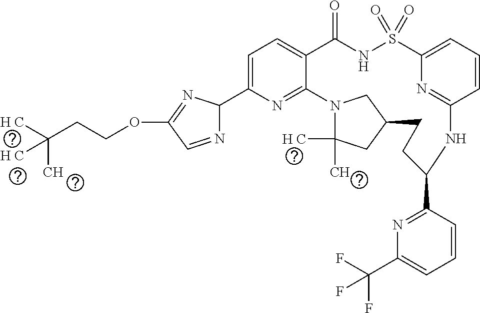 | +++ | ++ | | |
| 307 | 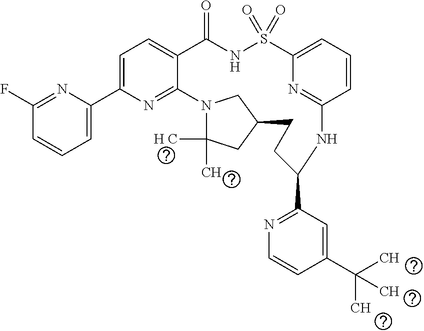 | +++ | +++ | | |
| 308 | 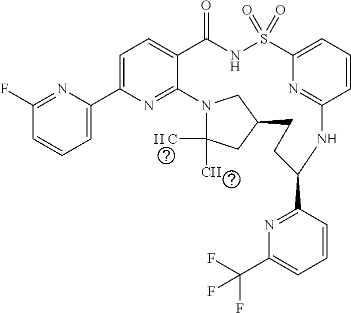 | +++ | ++ | | |
| 309 | 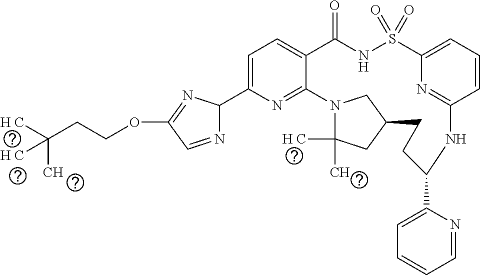 | +++ | ++ | | |
| 310 | 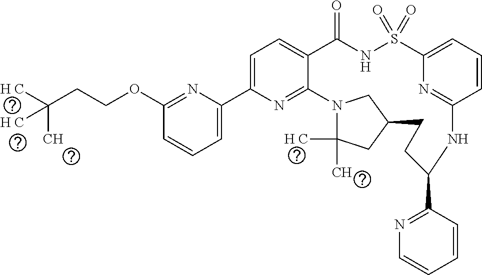 | +++ | ++ | | |
| 311 | 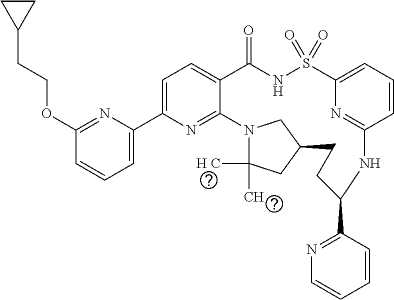 | +++ | +++ | | |
| 312 | 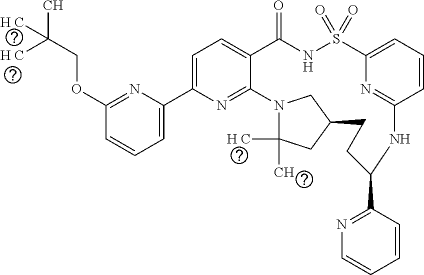 | +++ | +++ | | |
| 313 | 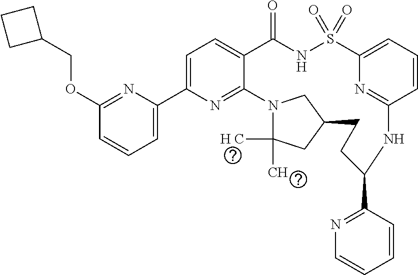 | +++ | +++ | | |
| 314 | 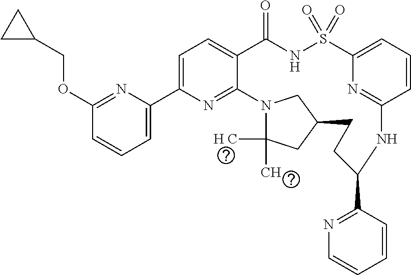 | +++ | +++ | | |
| 315 | 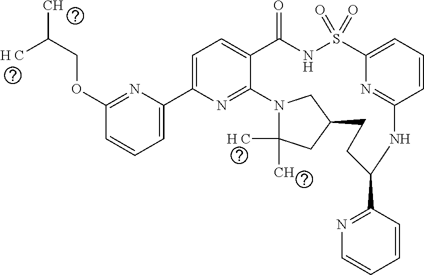 | +++ | +++ | | |
| 316 | 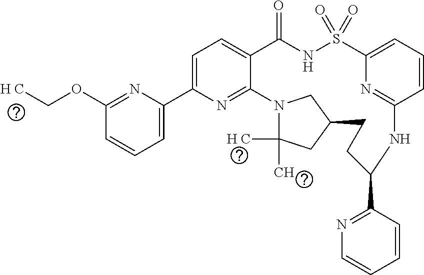 | +++ | +++ | | |
| 317 | 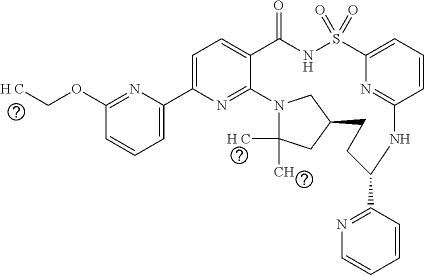 | +++ | ++ | | |
| 318 | 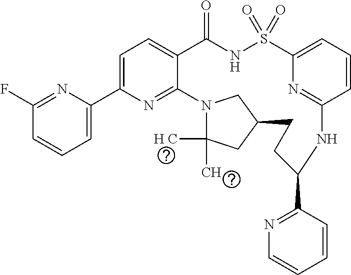 | +++ | +++ | | |
| 319 | 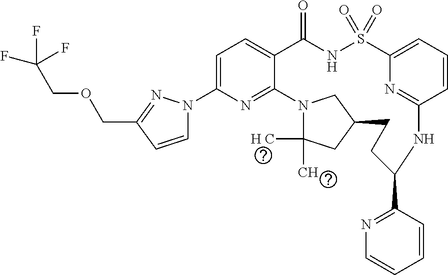 | +++ | +++ | | |
| 320 | 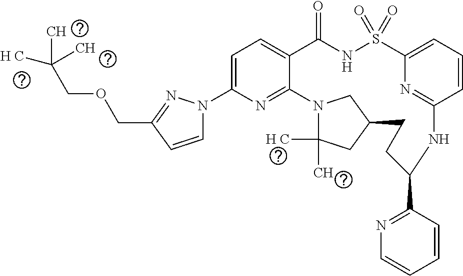 | +++ | +++ | | |
| 321 | 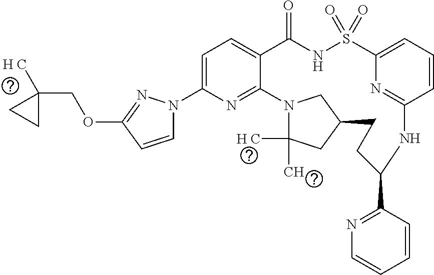 | +++ | +++ | | |
| 322 | 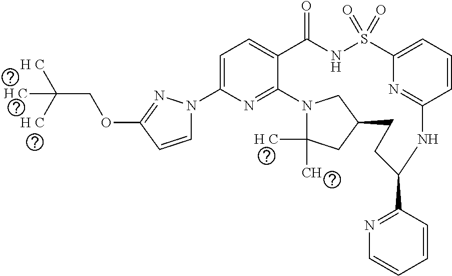 | +++ | ++ | | |
| 323 | 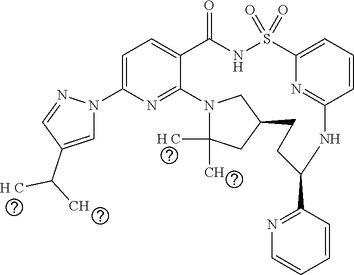 | +++ | ++ | | |
| 324 | 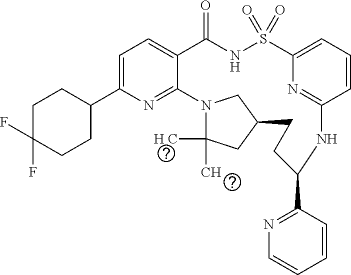 | +++ | +++ | | |
| 325 | 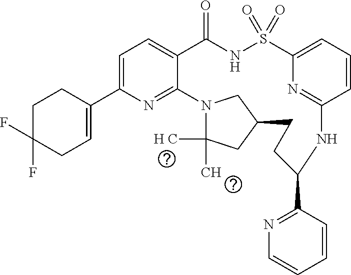 | +++ | +++ | | |
| 326 | 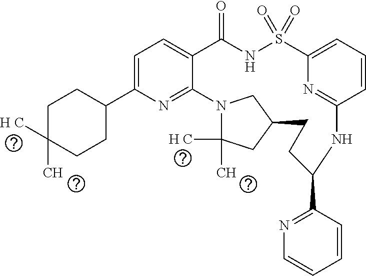 | +++ | +++ | | |
| 327 | 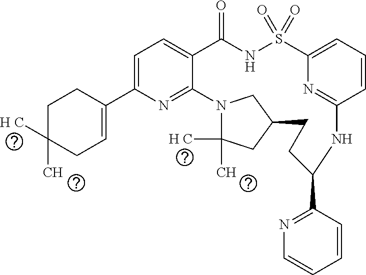 | +++ | +++ | | |
| 328 | 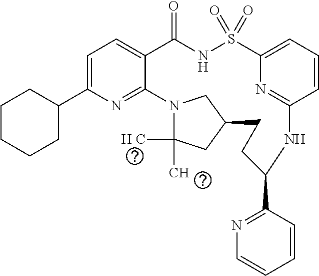 | +++ | +++ | | |
| 329 | 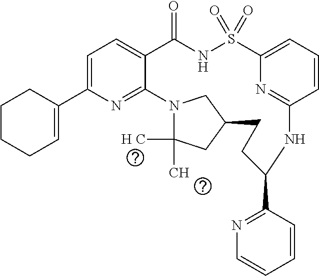 | +++ | +++ | | |
| 330 | 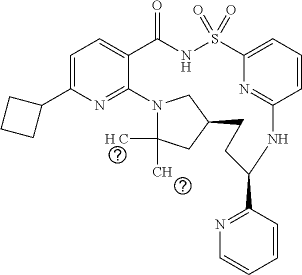 | +++ | +++ | | |
| 331 | 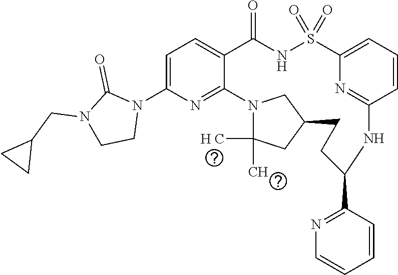 | +++ | +++ | | |
| 332 | 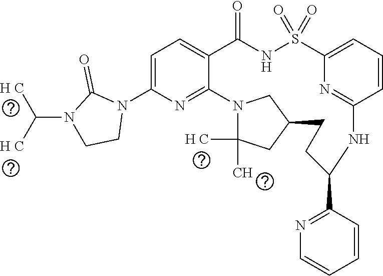 | ++ | +++ | | |
| 333 | 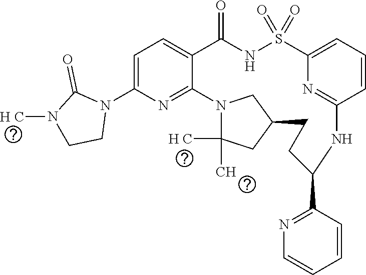 | ++ | +++ | | |
| 334 | 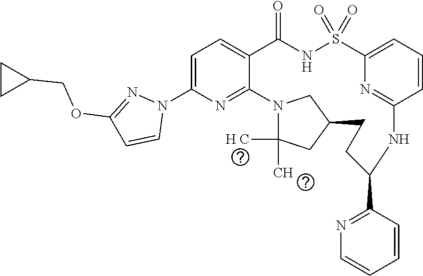 | +++ | +++ | | |
| 335 | 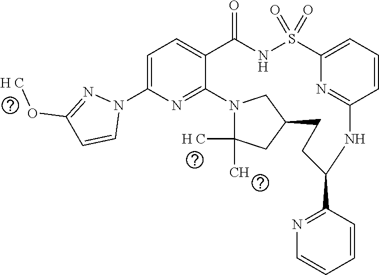 | +++ | +++ | | |
| 336 | 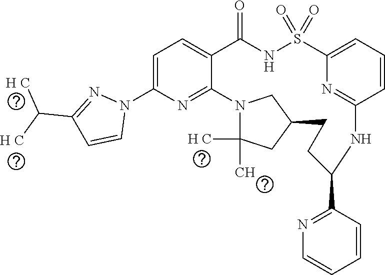 | +++ | +++ | | |
| 337 | 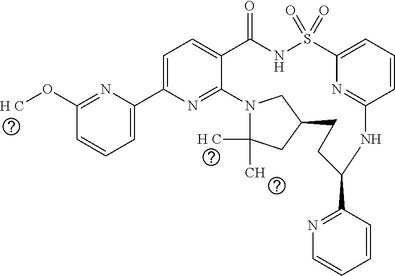 | +++ | +++ | | |
| 338 | 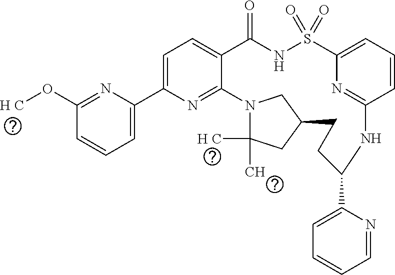 | +++ | +++ | | |
| 339 | 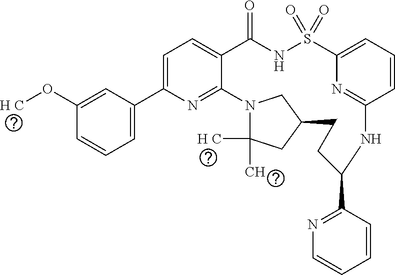 | +++ | +++ | | |
| 340 | 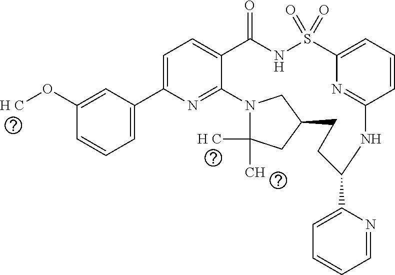 | ++ | ++ | | |
| 341 | 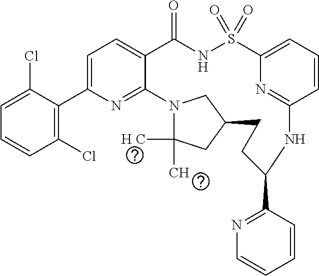 | +++ | +++ | | |
| 342 | 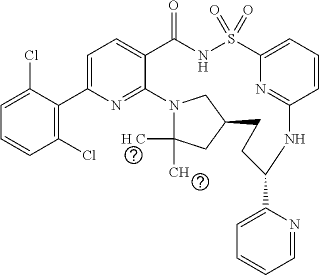 | +++ | ++ | | |
| 343 | 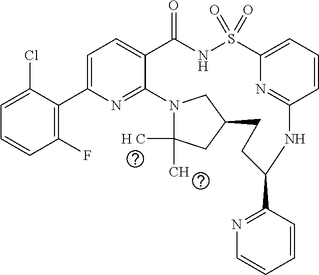 | +++ | +++ | | |
| 344 | 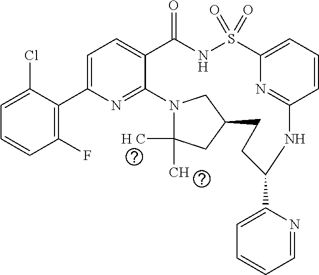 | +++ | ++ | | |
| 345 | 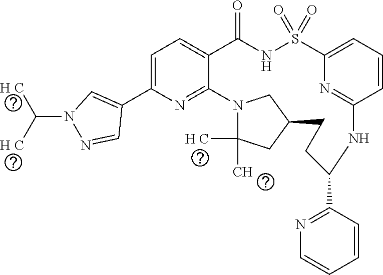 | +++ | ++ | | |
| 346 | 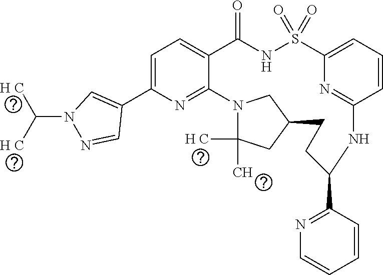 | +++ | ++ | | |
| 347 | 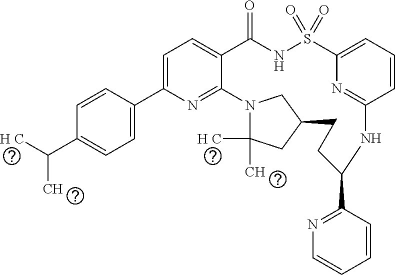 | +++ | +++ | | |
| 348 | 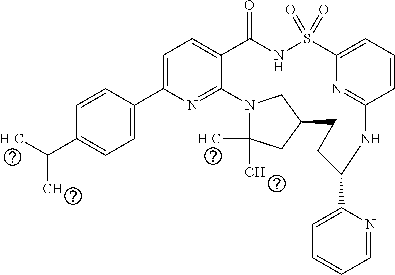 | +++ | ++ | | |
| 349 | 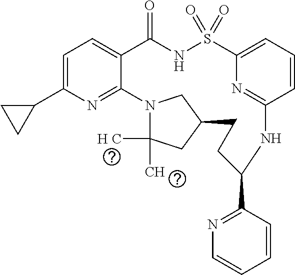 | +++ | +++ | | |
| 350 | 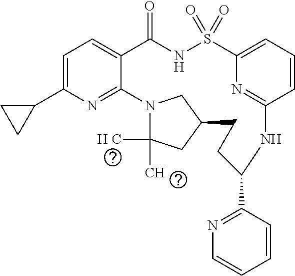 | +++ | +++ | | |
| 351 | 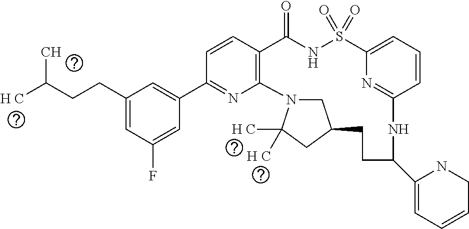 | +++ | +++ | | |
| 352 | 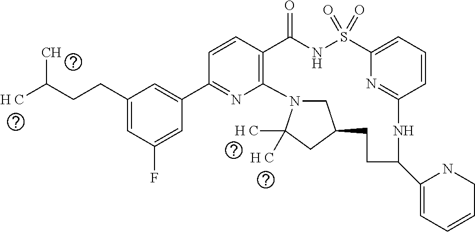 | +++ | ++ | | |
| 353 | 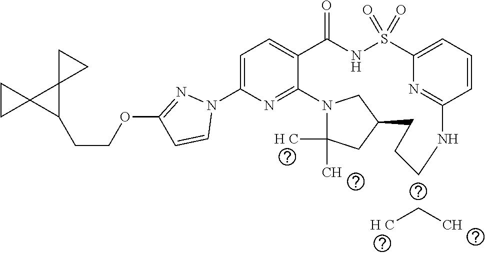 | +++ | +++ | | |
| 354 | 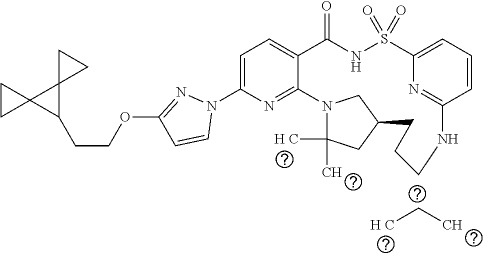 | +++ | +++ | | |
| 355 | 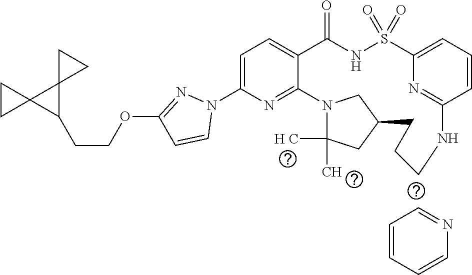 | +++ | +++ | | |
| 356 | 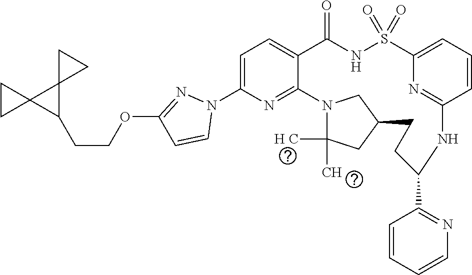 | +++ | +++ | | |
| 357 | 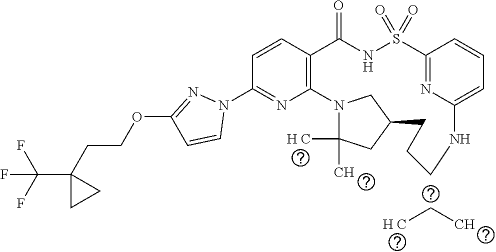 | +++ | +++ | ND | |
| 358 | 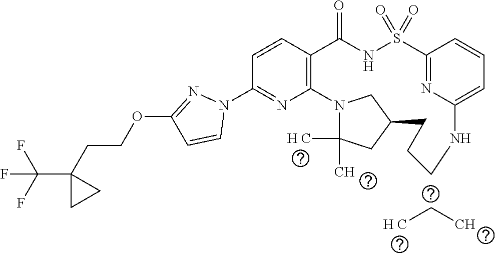 | +++ | +++ | +++ | +++ |
| 359 | 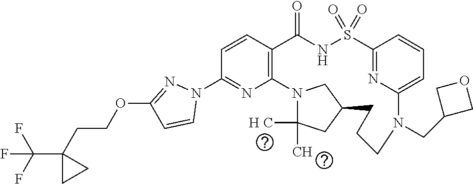 | | | +++ | +++ |
| 360 | 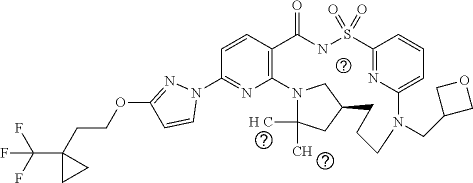 | | | ND | + |
| 361 | 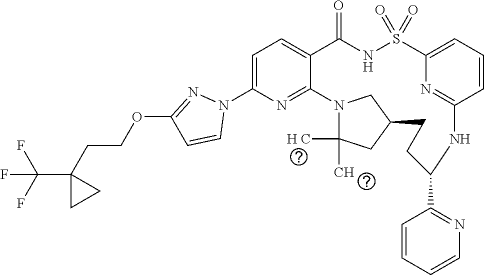 | +++ | +++ | +++ | +++ |
| 362 | 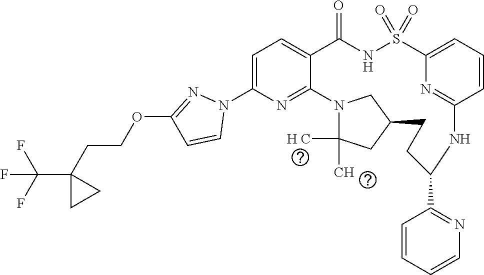 | +++ | +++ | +++ | +++ |
| 363 | 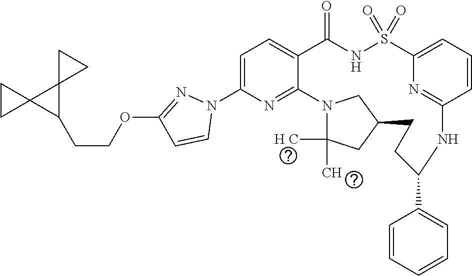 | +++ | +++ | +++ | +++ |
| 364 | 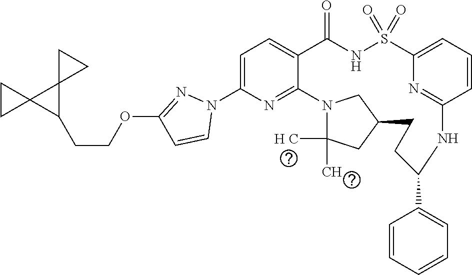 | +++ | +++ | +++ | +++ |
| 365 | 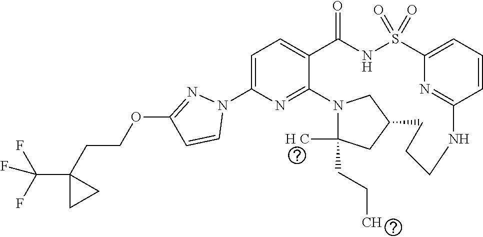 | | | ND | + |
| 366 | 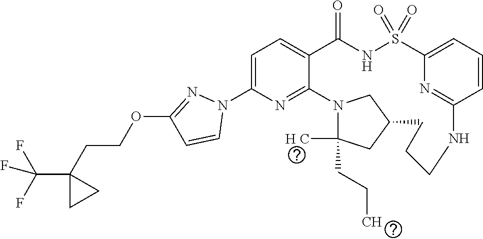 | | | ND | + |
| 367 | 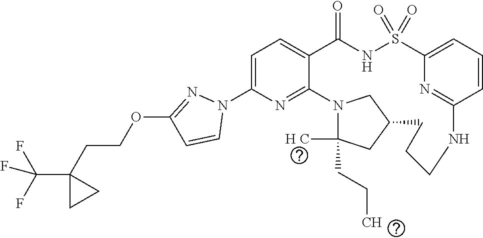 | +++ | +++ | +++ | ++ |
| 368 | 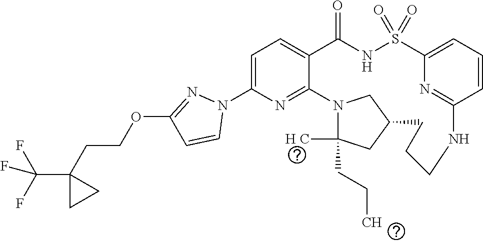 | | | +++ | +++ |
| 369 | 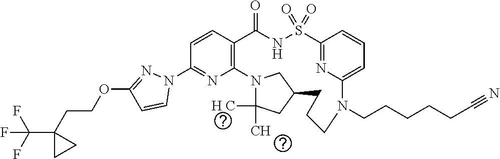 | | | +++ | ++ |
| 370 | 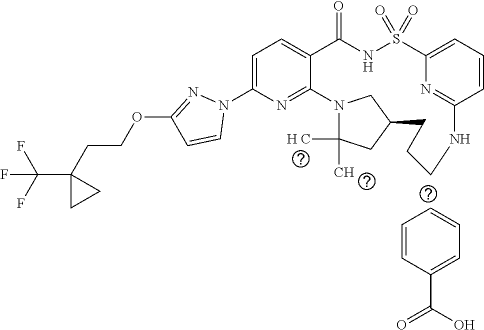 | | | ++ | +++ |
| 371 | 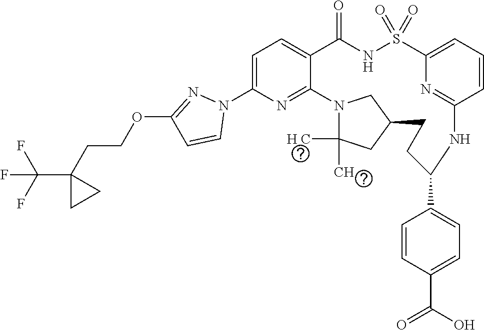 | | | +++ | +++ |
| 372 | 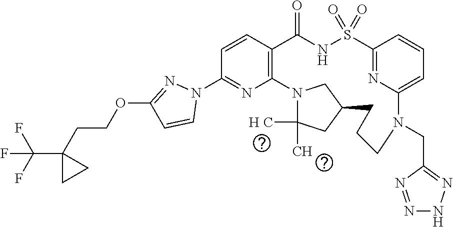 | | | ++ | +++ |
| 373 | 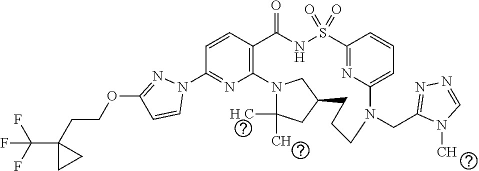 | | | +++ | ++ |
| 374 | 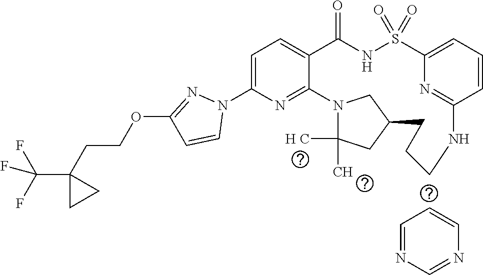 | | | +++ | +++ |
| 375 | 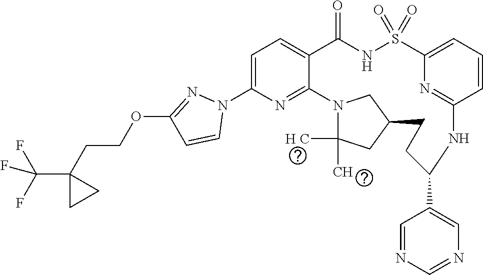 | | | +++ | +++ |
| 376 | 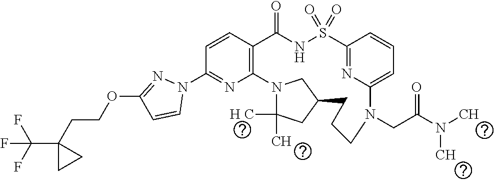 | | | +++ | ++ |
| 377 | 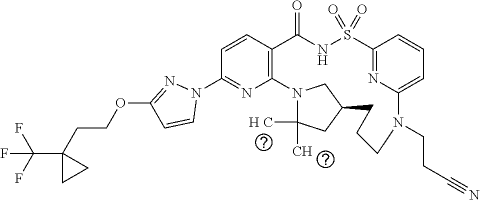 | | | +++ | +++ |
| 378 | 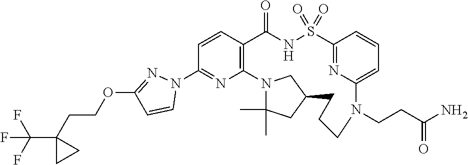 [SMILES: CC1(C)C[C@@H]2CCCN(CCC(N)=O)c3cccc(n3)S(=O)(=O)NC(=O)c3ccc(-n4ccc(OCCC5(C(F)(F)F)CC5)n4)nc3N1C2] | | | +++ | +++ |
| 379 | 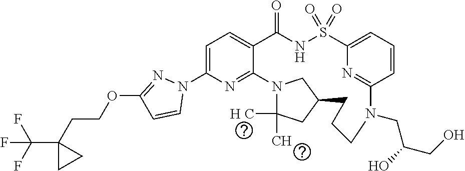 | | | +++ | +++ |
| 380 | 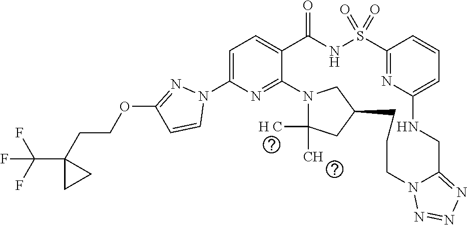 | | | +++ | +++ |
| 381 | 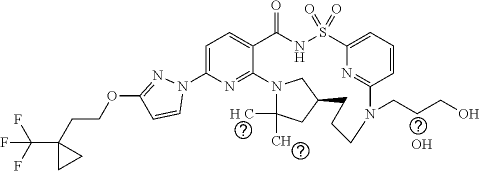 | | | +++ | +++ |
| 382 | 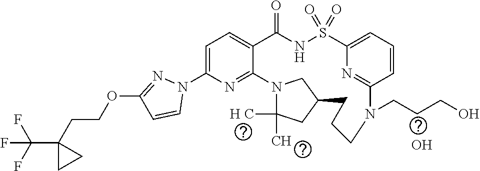 | | | +++ | +++ |
| 383 | 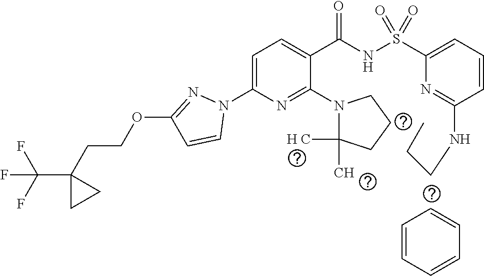 | +++ | ++ | +++ | +++ |
| 384 | 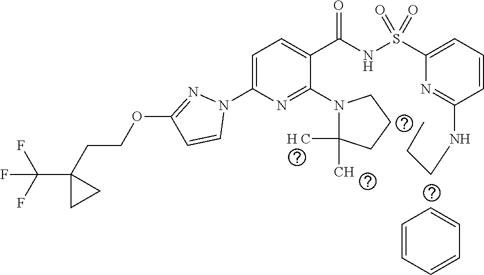 | +++ | + | +++ | ++ |
| 385 | 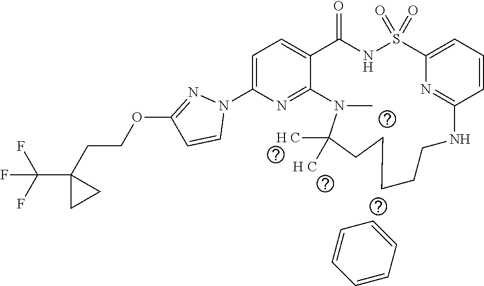 | +++ | ++ | +++ | ++ |
| 386 | 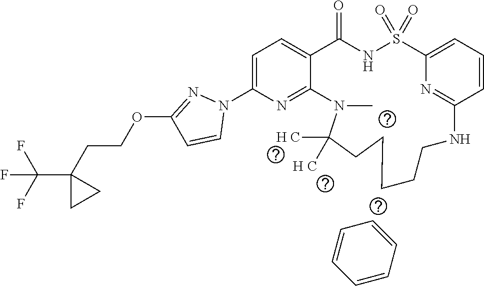 | | | +++ | ++ |
| 387 | 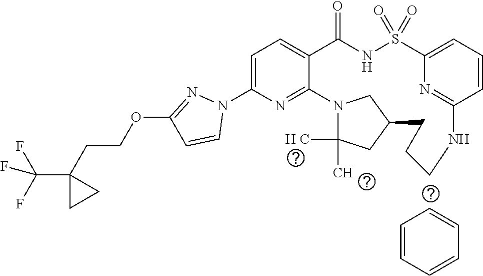 | +++ | +++ | +++ | +++ |
| 388 | 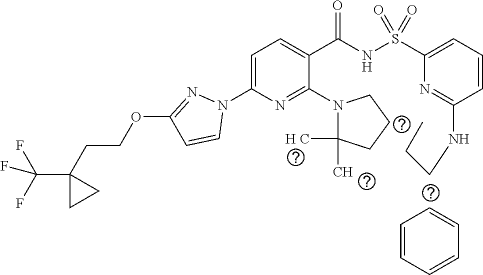 | ND | ND | ND | ND |
| 389 | 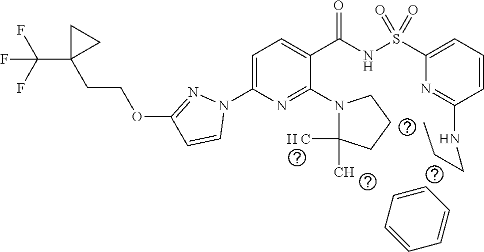 | +++ | +++ | +++ | +++ |
| 390 | 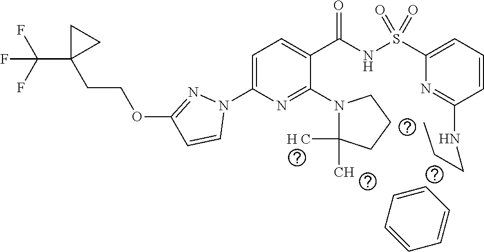 | | | +++ | +++ |
| 391 | 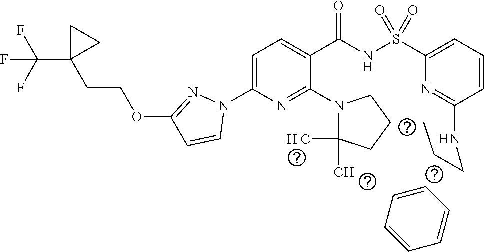 | +++ | +++ | +++ | +++ |
| 392 | 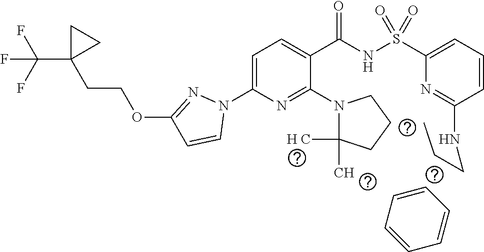 | | | +++ | ++ |
| 393 | 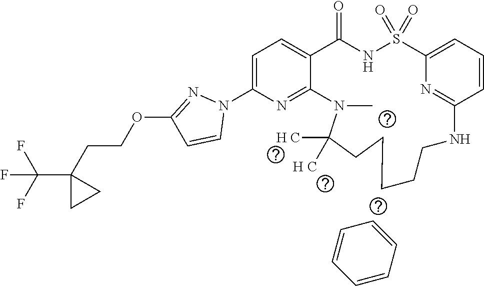 | +++ | +++ | +++ | +++ |
| 394 | 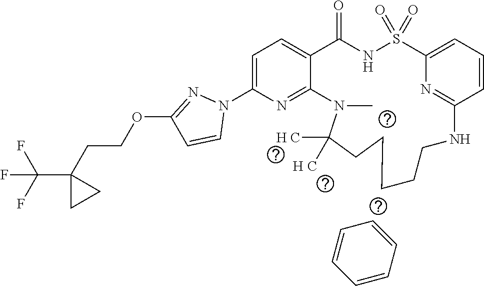 | | | ND | ND |
| 395 | 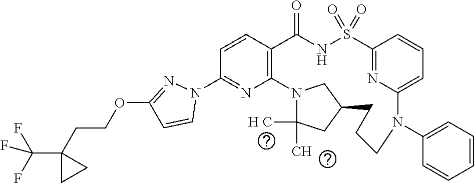 | | | +++ | ++ |
| 396 | 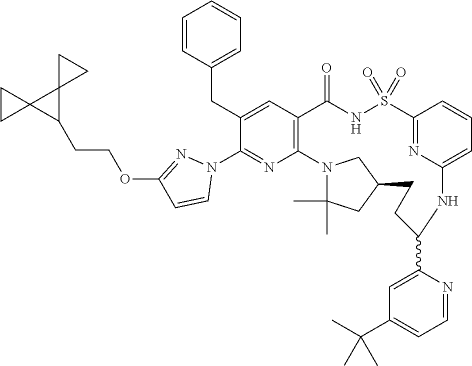 [SMILES: CC(C)(C)c1ccnc(C2CC[C@@H]3CN(c4nc(-n5ccc(OCCC6C7(CC7)C67CC7)n5)c(Cc5ccccc5)cc4C(=O)NS(=O)(=O)c4cccc(n4)N2)C(C)(C)C3)c1] | | | | |
| 397 | 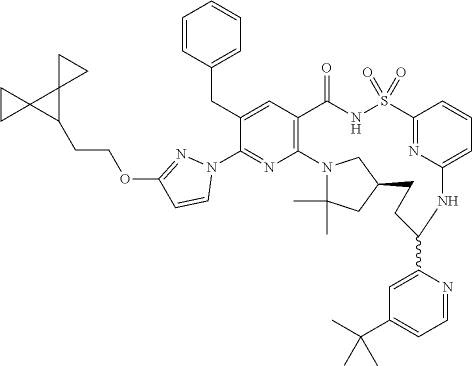 [SMILES: CC(C)(C)c1ccnc(C2CC[C@@H]3CN(c4nc(-n5ccc(OCCC6C7(CC7)C67CC7)n5)c(Cc5ccccc5)cc4C(=O)NS(=O)(=O)c4cccc(n4)N2)C(C)(C)C3)c1] | | | | |

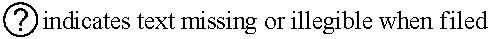

### Example 167: Compounds 398 to 436

Compounds 398 to 436, depicted in Table 4 were all demonstrated to have CFTR corrector activity.

TABLE 4
Compounds 398-436

| Com- | |
|---|---|
| pound | |
| Num- | |
| ber | Structure |
| Com- pound 398 | 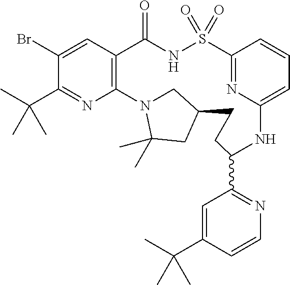 [SMILES: CC(C)(C)c1ccnc(C2CC[C@@H]3CN(c4nc(C(C)(C)C)c(Br)cc4C(=O)NS(=O)(=O)c4cccc(n4)N2)C(C)(C)C3)c1] |
| Com- pound 399 | 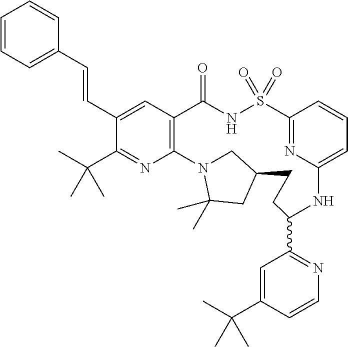 [SMILES: CC(C)(C)c1ccnc(C2CC[C@@H]3CN(c4nc(C(C)(C)C)c(/C=C/c5ccccc5)cc4C(=O)NS(=O)(=O)c4cccc(n4)N2)C(C)(C)C3)c1] |
| Com- pound 400 | 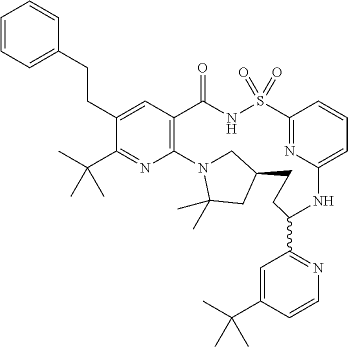 [SMILES: CC(C)(C)c1ccnc(C2CC[C@@H]3CN(c4nc(C(C)(C)C)c(CCc5ccccc5)cc4C(=O)NS(=O)(=O)c4cccc(n4)N2)C(C)(C)C3)c1] |
| Com- pound 401 | 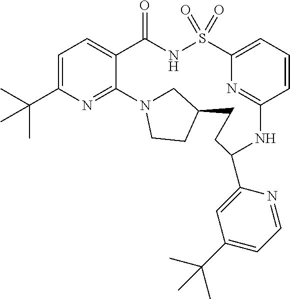 [SMILES: CC(C)(C)c1ccnc(C2CC[C@H]3CCN(C3)c3nc(C(C)(C)C)ccc3C(=O)NS(=O)(=O)c3cccc(n3)N2)c1] |
| Com- pound 402 | 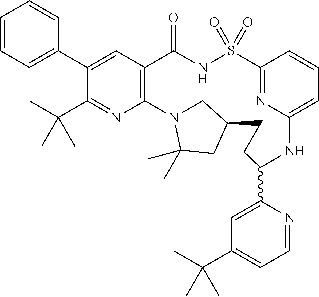 [SMILES: CC(C)(C)c1ccnc(C2CC[C@@H]3CN(c4nc(C(C)(C)C)c(-c5ccccc5)cc4C(=O)NS(=O)(=O)c4cccc(n4)N2)C(C)(C)C3)c1] |
| Com- pound 403 | 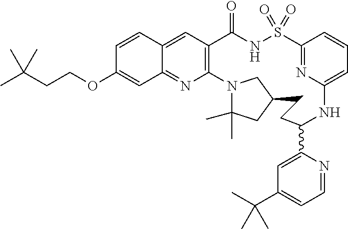 [SMILES: CC(C)(C)CCOc1ccc2cc3c(nc2c1)N1C[C@@H](CCC(c2cc(C(C)(C)C)ccn2)Nc2cccc(n2)S(=O)(=O)NC3=O)CC1(C)C] |
| Com- pound 404 | 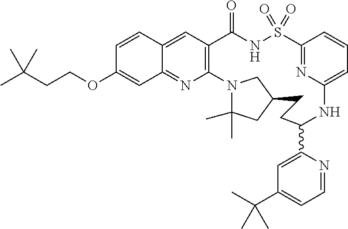 [SMILES: CC(C)(C)CCOc1ccc2cc3c(nc2c1)N1C[C@@H](CCC(c2cc(C(C)(C)C)ccn2)Nc2cccc(n2)S(=O)(=O)NC3=O)CC1(C)C] |
| Com- pound 405 | 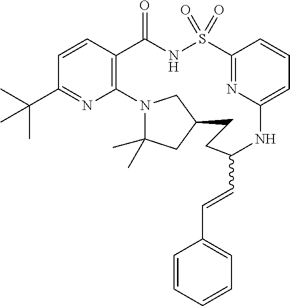 [SMILES: CC(C)(C)c1ccc2c(n1)N1C[C@@H](CCC(C=Cc3ccccc3)Nc3cccc(n3)S(=O)(=O)NC2=O)CC1(C)C] |
| Com- pound 406 | 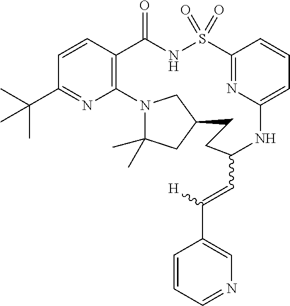 [SMILES: CC(C)(C)c1ccc2c(n1)N1C[C@@H](CCC(C=Cc3cccnc3)Nc3cccc(n3)S(=O)(=O)NC2=O)CC1(C)C] |
| Com- pound 407 | 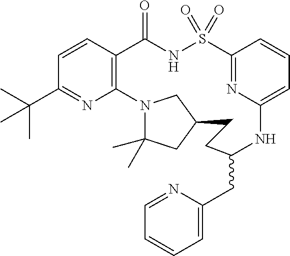 [SMILES: CC(C)(C)c1ccc2c(n1)N1C[C@@H](CCC(Cc3ccccn3)Nc3cccc(n3)S(=O)(=O)NC2=O)CC1(C)C] |
| Com- pound 408 | 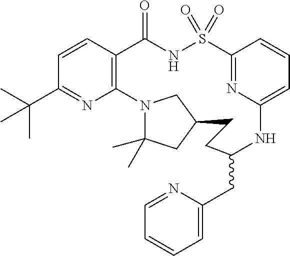 [SMILES: CC(C)(C)c1ccc2c(n1)N1C[C@@H](CCC(Cc3ccccn3)Nc3cccc(n3)S(=O)(=O)NC2=O)CC1(C)C] |
| Com- pound 409 | 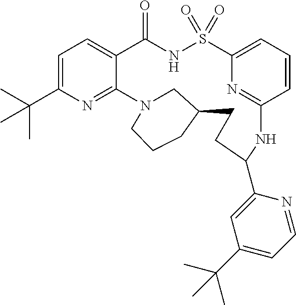 [SMILES: CC(C)(C)c1ccnc(C2CC[C@H]3CCCN(C3)c3nc(C(C)(C)C)ccc3C(=O)NS(=O)(=O)c3cccc(n3)N2)c1] |
| Com- pound 410 | 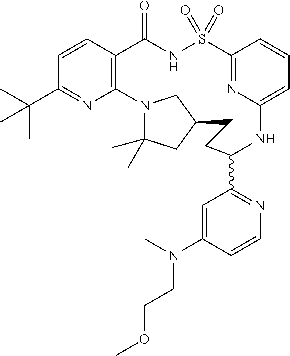 [SMILES: COCCN(C)c1ccnc(C2CC[C@@H]3CN(c4nc(C(C)(C)C)ccc4C(=O)NS(=O)(=O)c4cccc(n4)N2)C(C)(C)C3)c1] |
| Com- pound 411 | 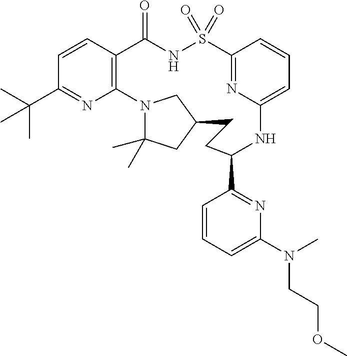 [SMILES: COCCN(C)c1cccc([C@H]2CC[C@@H]3CN(c4nc(C(C)(C)C)ccc4C(=O)NS(=O)(=O)c4cccc(n4)N2)C(C)(C)C3)n1] |
| Com- pound 412 | 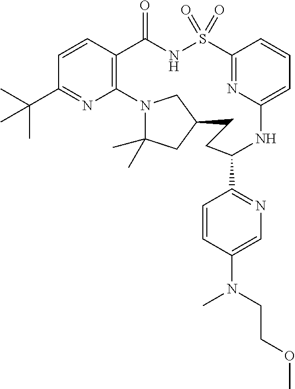 [SMILES: COCCN(C)c1ccc([C@@H]2CC[C@@H]3CN(c4nc(C(C)(C)C)ccc4C(=O)NS(=O)(=O)c4cccc(n4)N2)C(C)(C)C3)nc1] |
| Com- pound 413 | 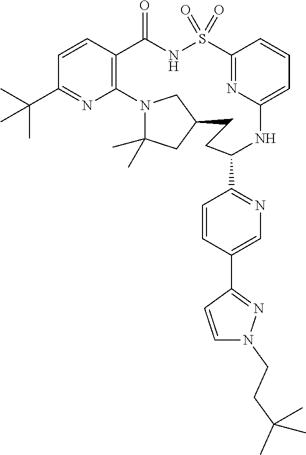 [SMILES: CC(C)(C)CCn1ccc(-c2ccc([C@@H]3CC[C@@H]4CN(c5nc(C(C)(C)C)ccc5C(=O)NS(=O)(=O)c5cccc(n5)N3)C(C)(C)C4)nc2)n1] |
| Com- pound 414 | 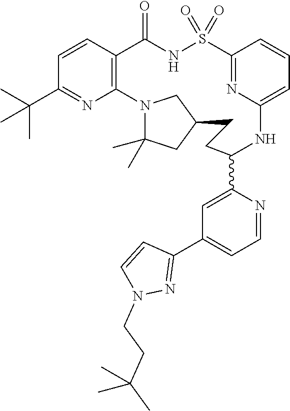 [SMILES: CC(C)(C)CCn1ccc(-c2ccnc(C3CC[C@@H]4CN(c5nc(C(C)(C)C)ccc5C(=O)NS(=O)(=O)c5cccc(n5)N3)C(C)(C)C4)c2)n1] |
| Com- pound 415 | 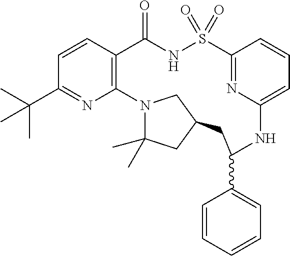 [SMILES: CC(C)(C)c1ccc2c(n1)N1C[C@@H](CC(c3ccccc3)Nc3cccc(n3)S(=O)(=O)NC2=O)CC1(C)C] |
| Com- pound 416 | 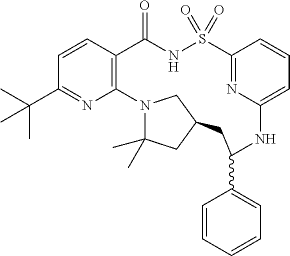 [SMILES: CC(C)(C)c1ccc2c(n1)N1C[C@@H](CC(c3ccccc3)Nc3cccc(n3)S(=O)(=O)NC2=O)CC1(C)C] |
| Com- pound 417 | 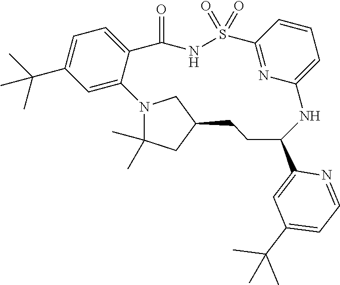 [SMILES: CC(C)(C)c1ccnc([C@H]2CC[C@@H]3CN(c4cc(C(C)(C)C)ccc4C(=O)NS(=O)(=O)c4cccc(n4)N2)C(C)(C)C3)c1] |
| Com- pound 418 | 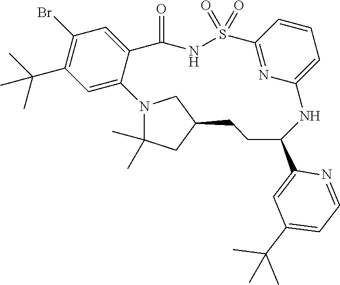 [SMILES: CC(C)(C)c1ccnc([C@H]2CC[C@@H]3CN(c4cc(C(C)(C)C)c(Br)cc4C(=O)NS(=O)(=O)c4cccc(n4)N2)C(C)(C)C3)c1] |
| Com- pound 419 | 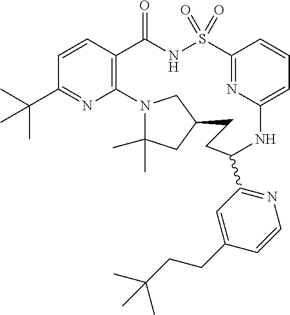 [SMILES: CC(C)(C)CCc1ccnc(C2CC[C@@H]3CN(c4nc(C(C)(C)C)ccc4C(=O)NS(=O)(=O)c4cccc(n4)N2)C(C)(C)C3)c1] |
| Com- pound 420 | 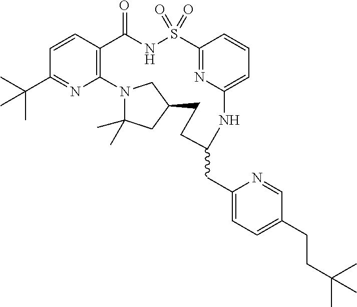 [SMILES: CC(C)(C)CCc1ccc(CC2CC[C@@H]3CN(c4nc(C(C)(C)C)ccc4C(=O)NS(=O)(=O)c4cccc(n4)N2)C(C)(C)C3)nc1] |
| Com- pound 421 | 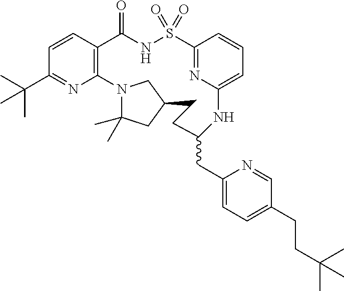 [SMILES: CC(C)(C)CCc1ccc(CC2CC[C@@H]3CN(c4nc(C(C)(C)C)ccc4C(=O)NS(=O)(=O)c4cccc(n4)N2)C(C)(C)C3)nc1] |
| Com- pound 422 | 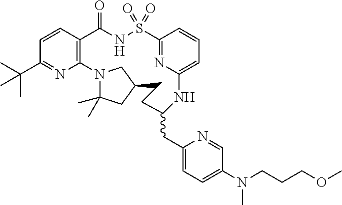 [SMILES: COCCCN(C)c1ccc(CC2CC[C@@H]3CN(c4nc(C(C)(C)C)ccc4C(=O)NS(=O)(=O)c4cccc(n4)N2)C(C)(C)C3)nc1] |
| Com- pound 423 | 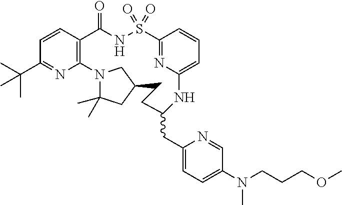 [SMILES: COCCCN(C)c1ccc(CC2CC[C@@H]3CN(c4nc(C(C)(C)C)ccc4C(=O)NS(=O)(=O)c4cccc(n4)N2)C(C)(C)C3)nc1] |
| Com- pound 424 | 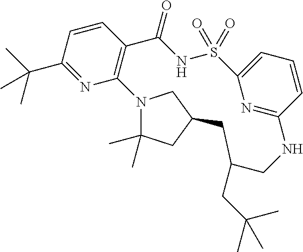 [SMILES: CC(C)(C)CC1CNc2cccc(n2)S(=O)(=O)NC(=O)c2ccc(C(C)(C)C)nc2N2C[C@@H](C1)CC2(C)C] |
| Com- pound 425 | 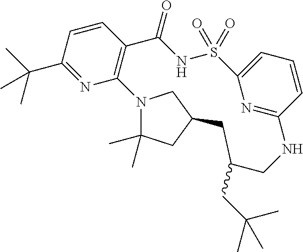 [SMILES: CC(C)(C)CC1CNc2cccc(n2)S(=O)(=O)NC(=O)c2ccc(C(C)(C)C)nc2N2C[C@@H](C1)CC2(C)C] |
| Com- pound 426 | 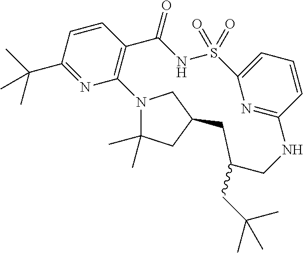 [SMILES: CC(C)(C)CC1CNc2cccc(n2)S(=O)(=O)NC(=O)c2ccc(C(C)(C)C)nc2N2C[C@@H](C1)CC2(C)C] |
| Com- pound 427 | 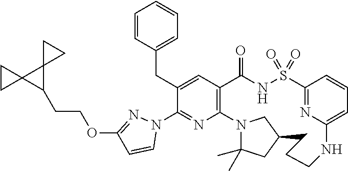 [SMILES: CC1(C)C[C@@H]2CCCNc3cccc(n3)S(=O)(=O)NC(=O)c3cc(Cc4ccccc4)c(-n4ccc(OCCC5C6(CC6)C56CC6)n4)nc3N1C2] |
| Com- pound 428a | 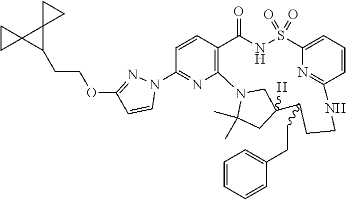 [SMILES: CC1(C)CC2CN1c1nc(-n3ccc(OCCC4C5(CC5)C45CC5)n3)ccc1C(=O)NS(=O)(=O)c1cccc(n1)NCCC2CCc1ccccc1] |
| Com- pound 428b | 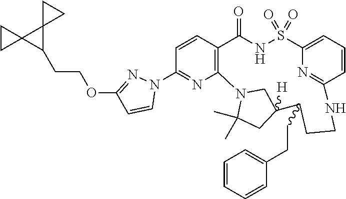 [SMILES: CC1(C)CC2CN1c1nc(-n3ccc(OCCC4C5(CC5)C45CC5)n3)ccc1C(=O)NS(=O)(=O)c1cccc(n1)NCCC2CCc1ccccc1] |
| Com- pound 429 | 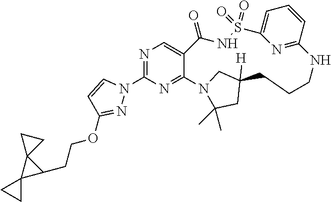 [SMILES: CC1(C)C[C@@H]2CCCNc3cccc(n3)S(=O)(=O)NC(=O)c3cnc(-n4ccc(OCCC5C6(CC6)C56CC6)n4)nc3N1C2] |
| Com- pound 430 | 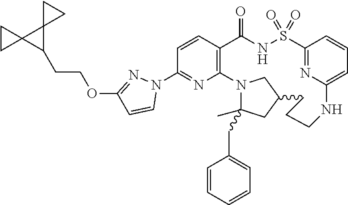 [SMILES: CC1(Cc2ccccc2)CC2CCCNc3cccc(n3)S(=O)(=O)NC(=O)c3ccc(-n4ccc(OCCC5C6(CC6)C56CC6)n4)nc3N1C2] |
| Com- pound 431 | 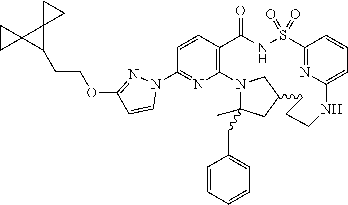 [SMILES: CC1(Cc2ccccc2)CC2CCCNc3cccc(n3)S(=O)(=O)NC(=O)c3ccc(-n4ccc(OCCC5C6(CC6)C56CC6)n4)nc3N1C2] |
| Com- pound 432 | 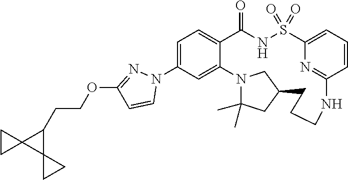 [SMILES: CC1(C)C[C@@H]2CCCNc3cccc(n3)S(=O)(=O)NC(=O)c3ccc(-n4ccc(OCCC5C6(CC6)C56CC6)n4)cc3N1C2] |
| Com- pound 433 | 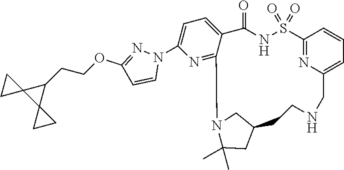 [SMILES: CC1(C)C[C@@H]2CCNCc3cccc(n3)S(=O)(=O)NC(=O)c3ccc(-n4ccc(OCCC5C6(CC6)C56CC6)n4)nc3N1C2] |
| Com- pound 434 | 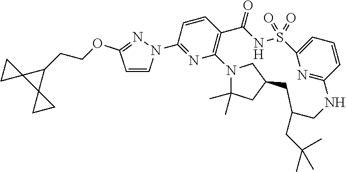 [SMILES: CC(C)(C)CC1CNc2cccc(n2)S(=O)(=O)NC(=O)c2ccc(-n3ccc(OCCC4C5(CC5)C45CC5)n3)nc2N2C[C@@H](C1)CC2(C)C] |
| Com- pound 435 | 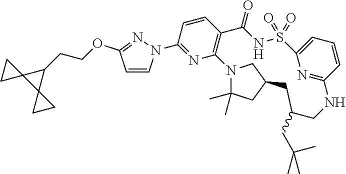 [SMILES: CC(C)(C)CC1CNc2cccc(n2)S(=O)(=O)NC(=O)c2ccc(-n3ccc(OCCC4C5(CC5)C45CC5)n3)nc2N2C[C@@H](C1)CC2(C)C] |
| Com- pound 436 | 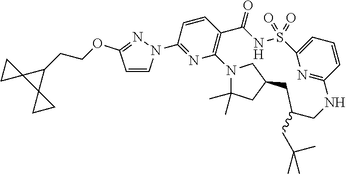 [SMILES: CC(C)(C)CC1CNc2cccc(n2)S(=O)(=O)NC(=O)c2ccc(-n3ccc(OCCC4C5(CC5)C45CC5)n3)nc2N2C[C@@H](C1)CC2(C)C] |

### Other Embodiments

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A compound of Formula (I): 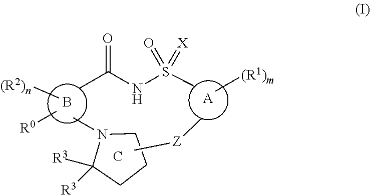
a tautomer thereof, a deuterated derivative of the compound or tautomer, or a pharmaceutically acceptable salt of any of the foregoing, wherein:
Ring A is a phenyl, indole, a 5-membered heteroaryl ring, or a 6-membered heteroaryl ring;
Ring B is a phenyl, pyridinyl, or pyrimidinyl ring;
X is O, NH, or an N(C₁-C₆ alkyl);
each R¹ is independently chosen from C₁-C₆ alkyl groups, C₁-C₆ alkoxyl groups, C₁-C₆ haloalkyl groups, C₁-C₆ haloalkoxyl groups, halogens, a cyano group, and a hydroxyl group, or two R¹ groups, together with the atoms to which they are attached, form a 5- to 6-membered heteroaryl or a 6-membered aryl ring;
m is 0, 1, 2, 3, or 4;
each R² is independently chosen from C₁-C₆ alkyl groups optionally substituted by phenyl or 5- or 6-membered heteroaryl, C₁-C₆ alkoxyl groups, C₁-C₆ haloalkyl groups, C₁-C₆ haloalkoxyl groups, halogens, a cyano group, and a hydroxyl group;
R⁰ is R¹¹ or 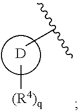
Ring D is a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, or a 3- to 8-membered cycloalkenyl;
each R⁴ is independently chosen from halogens, an oxo group, a hydroxyl group, a cyano group, and —(Y)ₖ—R⁷ groups, or optionally two R⁴, together with the atom(s) they are attached to, form a 5-6 membered cycloalkyl or heterocyclyl ring that is optionally and independently substituted with one or more groups chosen from halogens, C₁-C₆ alkyl groups, haloalkyl groups, a hydroxyl group, C₁-C₆ alkoxyl groups, and C₁-C₆ haloalkoxyl groups; wherein:
k is 0, 1, 2, 3, 4, 5, or 6;
each Y is independently chosen from C(R⁵)(R⁶) groups, —O—, and —NR^{a}— groups, wherein a heteroatom in —(Y)ₖ—R⁷ is not bonded to another heteroatom in —(Y)ₖ—R⁷, wherein:
each R⁵ and R⁶ is independently chosen from hydrogen, halogens, a hydroxyl group, C₁-C₆ alkyl groups, and C₃-5 cycloalkyl groups, or R⁵ and R⁶ on the same carbon together form a C₃-5 cycloalkyl group or oxo;
each of R⁵ and R⁶ is optionally independently substituted with one or more groups chosen from C₁-C₆ alkyl groups, C₁-C₆ haloalkyl groups, halogens, a hydroxyl group, C₁-C₆ alkoxyl groups, and C₁-C₆ haloalkoxyl groups; and
each R^{a} is independently chosen from hydrogen and C₁-C₆ alkyl groups;
and
R⁷ is chosen from hydrogen, halogens, a cyano group, and C₃-C₁₀ cycloalkyl groups optionally substituted with one or more groups chosen from C₁-C₆ alkyl groups, C₁-C₆haloalkyl groups, and halogens;
q is 1, 2, 3 or 4;
R¹¹ is chosen from hydrogen, halogen, C₁-C₆ alkyl groups, C₁-C₆ alkoxyl groups, C₁-C₆ haloalkyl groups, C₁-C₆ haloalkoxyl groups, C₂-C₆ alkenyl groups, C₂-C₆ alkynyl groups, benzyl, —O—(C₃-C₆ cycloalkyl), and a cyano group, each of which is substituted with 0, 1, 2, or 3 R¹² groups, or optionally one R² and R¹¹, together with the atoms to which they are attached, form a 5- to 6-membered cycloalkyl, a 5- to 6-membered heterocyclyl, or 6-membered aryl ring that is substituted with a phenyl ring, a 5-membered heterocyclyl ring, a 6-membered heterocyclyl ring, a 5-membered heteroaryl ring, a 6-membered heteroaryl ring, a 3- to 8-membered cycloalkyl ring, a 3- to 8-membered cycloalkenyl, or 0, 1, 2, 3 or 4 R² groups;
each R¹² is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl);
n is 0, 1, or 2;
each R³ is C₁-C₆ alkyl substituted by 0, 1, 2, 3, 4, 5, or 6 3- to 8-membered cycloalkyl rings or 5- or 6-membered aryl groups, or two R³ are joined to form a C₃-C₆ cycloaklyl ring;
Z is a divalent linker of formula (L)ᵣ, wherein:
r is 1, 2, 3, 4, 5, or 6;
each L is independently chosen from C(R⁸)(R⁹) groups, —O—, 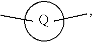
and —NR^{b}— groups, wherein a heteroatom in Z is not bonded to another heteroatom in Z, and wherein 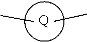
is a 5- or 6-membered heterocyclyl or a 5- or 6-membered heteroaryl, each of which is substituted with 0, 1, 2, 3, or 4 R¹⁰ groups; and wherein:
each of R⁸ and R⁹ is independently chosen from hydrogen, halogens, C₁-C₆ haloalkyl groups, C₁-C₆ alkyl groups, C₂-C₆ alkenyl, C₂-C₆ alkynyl, a hydroxyl group, C₁-C₆ alkoxyl groups, C₁-C₆ haloalkoxyl groups, CO₂H, C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups;
each R¹⁰ is independently chosen from halogen, hydroxyl, cyano, C₁-C₆ alkyl, C₂-C₆ alkenyl, C₂-C₆ alkynyl, —(C₁-C₆ alkyl)-O(C₁-C₆ alkyl), —(C₁-C₆ alkyl)-CO₂(C₁-C₆ alkyl), —(C₁-C₆ alkyl)-N(R^{x})(R^{y}), —(C₁-C₆ alkyl)-CO₂H, C₁-C₆ alkoxyl, —N(R^{x})(R^{y}), —CO—N(R^{x})(R^{y}), CO₂H, —CO₂(C₁-C₆ alkyl), —CO₂Bn, —CO(C₁-C₆ alkyl), phenyl, 5- to 6-membered heteroaryl, 4- to 6-membered heterocyclyl, and C₃-C₁₀ cycloalkyl, each of which is optionally and independently substituted with one or more groups chosen from halogens, cyano, C₁-C₆ alkyl groups, haloalkyl groups, a hydroxyl group, C₁-C₆ alkoxyl groups, C₁-C₆ haloalkoxyl groups, and —CO₂(C₁-C₆ alkyl),
or R⁸ and R⁹ on the same carbon together form an oxo;
each R^{b} is independently chosen from hydrogen, halogens, C₁-C₆ haloalkyl groups, C₁-C₆ alkyl groups, C₂-C₆ alkenyl, C₂-C₆ alkynyl, hydroxyl groups, C₁-C₆ alkoxyl groups, C₁-C₆ haloalkoxyl groups, —CO₂H, —C(O)N(R^{x})(R^{y}), phenyl, 3- to 8-membered cycloalkyl groups, 5- to 6-membered heteroaryl groups, and 5- to 6-membered heterocyclyl groups, each of which is substituted with 0, 1, 2, 3, 4 or 5 R¹⁰ groups, or optionally one R¹ and one R^{b}, together with the atoms to which they are attached, form a 5- to 6-membered heterocycloalkyl or a 5- to 6-membered heteroaryl ring, each of which is substituted with 0, 1, 2, 3, or 4 R¹⁰ groups; and
each R^{x} and R^{y} is independently chosen from hydrogen, C₁-C₆ alkyl, C₁-C₆ haloalkyl, C₄-C₉ heterocyclyl, 3- to 6-membered cycloalkyl groups, 5- to 6-membered heteroaryl groups, benzyl, —CO₂(C₁-C₆ alkyl), —CO(C₁-C₆ alkyl), wherein the C₁-C₆ alkyl is optionally substituted with —NMe₂, and wherein the C₄-C₉ heterocyclyl is optionally substituted with —(C₁-C₆ alkyl)-O(C₁-C₆ alkyl) or —CO₂(C₁-C₆ alkyl).

2. The compound, tautomer, deuterated derivative, or pharmaceutically acceptable salt according to claim 1, selected from compounds of Formulae (II), (II-Ai), (II-Aii), (II-Aiii), (II-Aiv), (II-Av), (II-Avi), (II-Bi), (II-Bii), (II-Biii), (II-Biv), (II-Bv), (II-Bvi), (II-Ci), (II-Cii), (II-Ciii), (II-Civ), (II-Cv), and (II-Cvi), (III), (III-Ai), (III-Aii), (III-Aiii), (III-Aiv), (III-Av), (III-Avi), (III-Avii), (III-Aviii), (III-Bi), (III-Bii), (III-Biii), (III-Biv), (III-Bv), (III-Bvi), (III-Ci), (III-Cii), (III-Ciii), (III-Civ), (III-Cv), and (III-Cvi), pharmaceutically acceptable salts thereof, and deuterated derivatives of any of the foregoing.

3. The compound, tautomer, deuterated derivative, or pharmaceutically acceptable salt according to claim 1, selected from Compounds 1-298 (Table 3A), Compounds 299-397 (Table 3B), Compounds 398-436 (Table 4), pharmaceutically acceptable salts thereof, and deuterated derivatives of any of the foregoing.

4. A pharmaceutical composition comprising the compound, tautomer, deuterated derivative, or pharmaceutically acceptable salt according to claim 1, and a pharmaceutically acceptable carrier.

5. The pharmaceutical composition of claim 4, further comprising one or more additional therapeutic agents.

6. The pharmaceutical composition of claim 5, wherein the one or more additional therapeutic agents are selected from tezacaftor, ivacaftor, D-ivacaftor, lumacaftor, and pharmaceutically acceptable salts thereof.

7. The pharmaceutical composition of claim 6, wherein the composition comprises tezacaftor and ivacaftor.

8. The pharmaceutical composition of claim 6 wherein the composition comprises tezacaftor and D-ivacaftor.

9. A method of treating cystic fibrosis comprising administering to a patient in need thereof the compound, tautomer, deuterated derivative, or pharmaceutically acceptable salt according to claim 1.

10. The method of claim 9, further comprising administering to the patient one or more additional therapeutic agents prior to, concurrent with, or subsequent to the compound, tautomer, deuterated derivative, or pharmaceutically acceptable salt.

11. The method of claim 10, wherein the one or more additional therapeutic agents is a compound selected from tezacaftor, ivacaftor, D-ivacaftor, lumacaftor, and pharmaceutically acceptable salts thereof.

12. The method of claim 11, wherein the one or more additional therapeutic agents are tezacaftor and ivacaftor.

13. The method of claim 11, wherein the one or more additional therapeutic agents are tezacaftor and D-ivacaftor.

14. A method of treating cystic fibrosis comprising administering to a patient in need thereof a pharmaceutical composition according to claim 4.

15. The method of claim 14, further comprising administering to the patient one or more additional therapeutic agents prior to, concurrent with, or subsequent to the pharmaceutical composition.

16. The method of claim 15, wherein the one or more additional therapeutic agents is a compound selected from tezacaftor, ivacaftor, D-ivacaftor, lumacaftor, and pharmaceutically acceptable salts thereof.

17. The method of claim 16, wherein the one or more additional therapeutic agents are tezacaftor and ivacaftor.

18. The method of claim 16, wherein the one or more additional therapeutic agents are tezacaftor and D-ivacaftor.
